SEQUENCE LISTING (144 sequences)

SEQ ID NO: 1 - protein, 115 aa, synthetic construct
EVQLVQSGAEVKKPGATVKISCKVSGYTFTEYTIHWVQQAPGKGLEWMGNINPNNGGTTYNQKFEDRVTITADTSTDTAYMELSSLRSEDTAVYYCATGWNFDYWGQGTLVTVSS

SEQ ID NO: 2 - protein, 115 aa, synthetic construct
EVQLVQSGAEVKKPGATVKISCKTSGYTFTEYTIHWVQQAPGKGLEWMGNINPNNGGTTYNQKFEDRVTITADTSTDTAYMELSSLRSEDTAVYYCATGWNFDYWGQGTLVTVSS

SEQ ID NO: 3 - protein, 115 aa, synthetic construct
EVQLVQSGAEVKKPGATVKISCKVSGYTFTEYTIHWVQQAPGKGLEWIGNINPNNGGTTYNQKFEDRVTITADTSTDTAYMELSSLRSEDTAVYYCATGWNFDYWGQGTLVTVSS

SEQ ID NO: 4 - protein, 115 aa, synthetic construct
EVQLVQSGAEVKKPGATVKISCKVSGYTFTEYTIHWVQQAPGKGLEWMGNINPNNGGTTYNQKFEDRVTITVDTSTDTAYMELSSLRSEDTAVYYCATGWNFDYWGQGTLVTVSS

SEQ ID NO: 5 - protein, 115 aa, synthetic construct
EVQLVQSGAEVKKPGATVKISCKVSGYTFTEYTIHWVQQAPGKGLEWMGNINPNNGGTTYNQKFEDRVTITADKSTDTAYMELSSLRSEDTAVYYCATGWNFDYWGQGTLVTVSS

SEQ ID NO: 6 - protein, 115 aa, synthetic construct
EVQLVQSGAEVKKPGATVKISCKVSGYTFTEYTIHWVQQAPGKGLEWMGNINPNNGGTTYNQKFEDRVTITADTSTSTAYMELSSLRSEDTAVYYCATGWNFDYWGQGTLVTVSS

SEQ ID NO: 7 - protein, 115 aa, synthetic construct
EVQLVQSGAEVKKPGATVKISCKVSGYTFTEYTIHWVQQAPGKGLEWMGNINPNNGGTTYNQKFEDRVTITADTSTDTAYMELSSLRSEDTAVYYCAAGWNFDYWGQGTLVTVSS

SEQ ID NO: 8 - protein, 115 aa, synthetic construct
EVQLVQSGAEVKKPGATVKISCKVSGYTFTEYTIHWVQQAPGKGLEWIGNINPNNGGTTYNQKFEDRVTITADTSTDTAYMELSSLRSEDTAVYYCAAGWNFDYWGQGTLVTVSS

SEQ ID NO: 9 - protein, 115 aa, synthetic construct
EVQLVQSGAEVKKPGATVKISCKTSGYTFTEYTIHWVQQAPGKGLEWIGNINPNNGGTTYNQKFEDRVTITADKSTSTAYMELSSLRSEDTAVYYCAAGWNFDYWGQGTLVTVSS

SEQ ID NO: 10 - protein, 115 aa, synthetic construct
EVQLVQSGAEVKKPGATVKISCKTSGYTFTEYTIHWVQQAPGKGLEWIGNINPNNGGTTYNQKFEDRVTITADTSTDTAYMELSSLRSEDTAVYYCAAGWNFDYWGQGTLVTVSS

SEQ ID NO: 11 - protein, 115 aa, synthetic construct
EVQLVQSGAEVKKPGATVKISCKVSGYTFTEYTIHWVQQAPGKGLEWMGNINPNNGGTTYNQKFEGRVTITADTSTDTAYMELSSLRSEDTAVYYCATGWNFDYWGQGTLVTVSS

SEQ ID NO: 12 - protein, 115 aa, synthetic construct
EVQLVQSGAEVKKPGATVKISCKVSGYTFTEYTIHWVQQAPGKGLEWMGNINPNNGGTTYNQKFQGRVTITADTSTDTAYMELSSLRSEDTAVYYCATGWNFDYWGQGTLVTVSS

SEQ ID NO: 13 - protein, 115 aa, synthetic construct
EVQLVQSGAEVKKPGATVKISCKVSGYTFTEYTIHWVQQAPGKGLEWMGNINPNNGGTTYNEKFQGRVTITADTSTDTAYMELSSLRSEDTAVYYCATGWNFDYWGQGTLVTVSS

SEQ ID NO: 14 - protein, 115 aa, synthetic construct
EVQLVQSGAEVKKPGATVKISCKVSGYTFTEYTIHWVQQAPGKGLEWMGNINPNNGGTTYAEKFQGRVTITADTSTDTAYMELSSLRSEDTAVYYCATGWNFDYWGQGTLVTVSS

SEQ ID NO: 15 - protein, 107 aa, synthetic construct
DIQMTQSPSSLSASVGDRVTITCKASQDVGTAVDWYQQKPGKAPKLLIYWASTRHTGVPSRFSGSGSGTDFTLTISSLQPEDFATYYCQQYNSYPLTFGQGTKLEIK

SEQ ID NO: 16 - protein, 107 aa, synthetic construct
DIQMTQSPSSLSASVGDRVTITCRASQDVGTAVDWYQQKPGKAPKLLIYWASTRHTGVPSRFSGSGSGTDFTLTISSLQPEDFATYYCQQYNSYPLTFGQGTKLEIK

SEQ ID NO: 17 - protein, 107 aa, synthetic construct
DIQMTQSPSSLSASVGDRVTITCRASQDVGTALNWYQQKPGKAPKLLIYWASTRHTGVPSRFSGSGSGTDFTLTISSLQPEDFATYYCQQYNSYPLTFGQGTKLEIK

SEQ ID NO: 18 - protein, 107 aa, synthetic construct
DIQMTQSPSSLSASVGDRVTITCKASQDVGTAVDWYQQKPGKAPKLLIYWASTRHSGVPSRFSGSGSGTDFTLTISSLQPEDFATYYCQQYNSYPLTFGQGTKLEIK

SEQ ID NO: 19 - protein, 107 aa, synthetic construct
DIQMTQSPSSLSASVGDRVTITCRASQDVGTAVDWYQQKPGKAPKLLIYWASTRHSGVPSRFSGSGSGTDFTLTISSLQPEDFATYYCQQYNSYPLTFGQGTKLEIK

SEQ ID NO: 20 - protein, 107 aa, synthetic construct
DIQMTQSPSSLSASVGDRVTITCRASQDVGTALNWYQQKPGKAPKLLIYWASTRHSGVPSRFSGSGSGTDFTLTISSLQPEDFATYYCQQYNSYPLTFGQGTKLEIK

SEQ ID NO: 21 - protein, 5 aa, synthetic construct
EYTIH

SEQ ID NO: 22 - protein, 17 aa, synthetic construct
NINPNNGGTTYAEKFQG

SEQ ID NO: 23 - protein, 17 aa, synthetic construct
NINPNNGGTTYNEKFQG

SEQ ID NO: 24 - protein, 17 aa, synthetic construct
NINPNNGGTTYNQKFQG

SEQ ID NO: 25 - protein, 17 aa, synthetic construct
NINPNNGGTTYNQKFEG

SEQ ID NO: 26 - protein, 17 aa, synthetic construct
NINPNNGGTTYNQKFED

SEQ ID NO: 27 - protein, 6 aa, synthetic construct
GWNFDY

SEQ ID NO: 28 - protein, 8 aa, synthetic construct
GYTFTEYT

SEQ ID NO: 29 - protein, 8 aa, synthetic construct
INPNNGGT

SEQ ID NO: 30 - protein, 8 aa, synthetic construct
ATGWNFDY

SEQ ID NO: 31 - protein, 8 aa, synthetic construct
AAGWNFDY

SEQ ID NO: 32 - protein, 11 aa, synthetic construct
RASQDVGTAVD

SEQ ID NO: 33 - protein, 11 aa, synthetic construct
KASQDVGTAVD

SEQ ID NO: 34 - protein, 11 aa, synthetic construct
RASQDVGTALN

SEQ ID NO: 35 - protein, 7 aa, synthetic construct
WASTRHS

SEQ ID NO: 36 - protein, 7 aa, synthetic construct
WASTRHT

SEQ ID NO: 37 - protein, 9 aa, synthetic construct
QQYNSYPLT

SEQ ID NO: 38 - protein, 6 aa, synthetic construct
QDVGTA

SEQ ID NO: 39 - skipped

SEQ ID NO: 40 - protein, 115 aa, synthetic construct
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 41 - protein, 107 aa, synthetic construct
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 42 - protein, 115 aa, synthetic construct
  VARIANT 24: note = V or T
  VARIANT 48: note = M or I
  VARIANT 61: note = A or N
  VARIANT 62: note = E or Q
  VARIANT 65: note = Q or E
  VARIANT 66: note = G or D
  VARIANT 72: note = A or V
  VARIANT 74: note = T or K
  VARIANT 77: note = D or S
  VARIANT 98: note = T or A
EVQLVQSGAEVKKPGATVKISCKXSGYTFTEYTIHWVQQAPGKGLEWXGNINPNNGGTTYXXKFXXRVTITXDXSTXTAYMELSSLRSEDTAVYYCAXGWNFDYWGQGTLLTVSS

SEQ ID NO: 43 - protein, 107 aa, synthetic construct
  VARIANT 24: note = R or K
  VARIANT 33: note = L or V
  VARIANT 34: note = N or D
  VARIANT 56: note = S or T
DIQMTQSPSSLSASVGDRVTITCXASQDVGTAXXWYQQKPGKAPKLLIYWASTRHXGVPSRFSGSGSGTDFTLTISSLQPEDFATYYCQQYNSYPLTFGQGTKLEIK

SEQ ID NO: 44 - protein, 17 aa, synthetic construct
  VARIANT 12: note = A or N
  VARIANT 13: note = E or Q
  VARIANT 16: note = Q or E
  VARIANT 17: note = G or D
NINPNNGGTTYXXKFXX

SEQ ID NO: 45 - protein, 11 aa, synthetic construct
  VARIANT 1: note = R or K
  VARIANT 10: note = V or L
  VARIANT 11: note = D or N
XASQDVGTAXX

SEQ ID NO: 46 - protein, 7 aa, synthetic construct
  VARIANT 7: note = S or T
WASTRHX

SEQ ID NO: 47 - protein, 445 aa, Homo sapiens
EVQLVQSGAEVKKPGATVKISCKVSGYTFTEYTIHWVQQAPGKGLEWMGNINPNNGGTTYNQKFEDRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 445 aa, Homo sapiens
EVQLVQSGAEVKKPGATVKISCKTSGYTFTEYTIHWVQQAPGKGLEWMGNINPNNGGTTYNQKFEDRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 445 aa, Homo sapiens
EVQLVQSGAEVKKPGATVKISCKVSGYTFTEYTIHWVQQAPGKGLEWIGNINPNNGGTTYNQKFEDRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 445 aa, Homo sapiens
EVQLVQSGAEVKKPGATVKISCKVSGYTFTEYTIHWVQQAPGKGLEWMGNINPNNGGTTYNQKFEDRVTITVDTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 445 aa, Homo sapiens
EVQLVQSGAEVKKPGATVKISCKVSGYTFTEYTIHWVQQAPGKGLEWMGNINPNNGGTTYNQKFEDRVTITADKSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 445 aa, Homo sapiens
EVQLVQSGAEVKKPGATVKISCKVSGYTFTEYTIHWVQQAPGKGLEWMGNINPNNGGTTYNQKFEDRVTITADTSTSTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 445 aa, Homo sapiens
EVQLVQSGAEVKKPGATVKISCKVSGYTFTEYTIHWVQQAPGKGLEWMGNINPNNGGTTYNQKFEDRVTITADTSTDTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 445 aa, Homo sapiens
EVQLVQSGAEVKKPGATVKISCKVSGYTFTEYTIHWVQQAPGKGLEWIGNINPNNGGTTYNQKFEDRVTITADTSTDTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 445 aa, Homo sapiens
EVQLVQSGAEVKKPGATVKISCKTSGYTFTEYTIHWVQQAPGKGLEWIGNINPNNGGTTYNQKFEDRVTITADKSTSTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 445 aa, Homo sapiens
EVQLVQSGAEVKKPGATVKISCKTSGYTFTEYTIHWVQQAPGKGLEWIGNINPNNGGTTYNQKFEDRVTITADTSTDTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 445 aa, Homo sapiens
EVQLVQSGAEVKKPGATVKISCKVSGYTFTEYTIHWVQQAPGKGLEWMGNINPNNGGTTYNQKFEGRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 445 aa, Homo sapiens
EVQLVQSGAEVKKPGATVKISCKVSGYTFTEYTIHWVQQAPGKGLEWMGNINPNNGGTTYNQKFQGRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 445 aa, Homo sapiens
EVQLVQSGAEVKKPGATVKISCKVSGYTFTEYTIHWVQQAPGKGLEWMGNINPNNGGTTYNEKFQGRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 445 aa, Homo sapiens
EVQLVQSGAEVKKPGATVKISCKVSGYTFTEYTIHWVQQAPGKGLEWMGNINPNNGGTTYAEKFQGRVTITADTSTDTAYMELSSLRSEDTAVYYCATGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, Homo sapiens
DIQMTQSPSSLSASVGDRVTITCKASQDVGTAVDWYQQKPGKAPKLLIYWASTRHTGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, Homo sapiens
DIQMTQSPSSLSASVGDRVTITCRASQDVGTAVDWYQQKPGKAPKLLIYWASTRHTGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 214 aa, Homo sapiens
DIQMTQSPSSLSASVGDRVTITCRASQDVGTALNWYQQKPGKAPKLLIYWASTRHTGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 214 aa, Homo sapiens
DIQMTQSPSSLSASVGDRVTITCKASQDVGTAVDWYQQKPGKAPKLLIYWASTRHSGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 214 aa, Homo sapiens
DIQMTQSPSSLSASVGDRVTITCRASQDVGTAVDWYQQKPGKAPKLLIYWASTRHSGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, Homo sapiens
DIQMTQSPSSLSASVGDRVTITCRASQDVGTALNWYQQKPGKAPKLLIYWASTRHSGVPSRFSGSGSGTDFTLTISSLQPEDFATYYCQQYNSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 68 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 69 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 105 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 71 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 115 aa, synthetic construct
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 73 - protein, 107 aa, synthetic construct
DIVMTQSHKFMSTSVGDRVSIICKASQDVGTAVDWYQQKPGQSPKLLIYWASTRHTGVPDRFTGSGSGTDFTLTTTNVQSEDLADYFCQQYNSYPLTFGAGTMLDLK

SEQ ID NO: 74 - DNA, 15 bp, synthetic construct
  source 1..15: mol_type = other DNA
gagtacaccattcac

SEQ ID NO: 75 - DNA, 51 bp, synthetic construct
  source 1..51: mol_type = other DNA
aacatcaaccccaacaacggcggcaccacctacgccgaaaaattccagggc

SEQ ID NO: 76 - DNA, 51 bp, synthetic construct
  source 1..51: mol_type = other DNA
aacatcaaccccaacaacggcggcaccacctacaacgaaaaattccagggc

SEQ ID NO: 77 - DNA, 51 bp, synthetic construct
  source 1..51: mol_type = other DNA
aacatcaaccccaacaacggcggcaccacctacaaccagaaattccagggc

SEQ ID NO: 78 - DNA, 51 bp, synthetic construct
  source 1..51: mol_type = other DNA
aacatcaaccccaacaacggcggcaccacctacaaccagaaattcgagggc

SEQ ID NO: 79 - DNA, 51 bp, synthetic construct
  source 1..51: mol_type = other DNA
aacatcaaccccaacaacggcggcaccacctacaaccagaaattcgaggac

SEQ ID NO: 80 - DNA, 18 bp, synthetic construct
  source 1..18: mol_type = other DNA
ggctggaacttcgactac

SEQ ID NO: 81 - DNA, 33 bp, synthetic construct
  source 1..33: mol_type = other DNA
agagccagccaggatgtgggcaccgctgttgac

SEQ ID NO: 82 - DNA, 33 bp, synthetic construct
  source 1..33: mol_type = other DNA
aaggccagccaggatgtgggcaccgctgttgac

SEQ ID NO: 83 - DNA, 33 bp, synthetic construct
  source 1..33: mol_type = other DNA
agagccagccaggatgtgggcaccgctctgaac

SEQ ID NO: 84 - DNA, 21 bp, synthetic construct
  source 1..21: mol_type = other DNA
tgggccagcacaagacacagc

SEQ ID NO: 85 - DNA, 21 bp, synthetic construct
  source 1..21: mol_type = other DNA
tgggccagcacaagacacaca

SEQ ID NO: 86 - DNA, 27 bp, synthetic construct
  source 1..27: mol_type = other DNA
cagcagtacaacagctaccctctgacc

SEQ ID NO: 87 - DNA, 24 bp, synthetic construct
  source 1..24: mol_type = other DNA
ggctacaccttcaccgagtacacc

SEQ ID NO: 88 - DNA, 24 bp, synthetic construct
  source 1..24: mol_type = other DNA
atcaaccccaacaacggcggcacc

SEQ ID NO: 89 - DNA, 24 bp, synthetic construct
  source 1..24: mol_type = other DNA
gccaccggctggaacttcgactac

SEQ ID NO: 90 - DNA, 24 bp, synthetic construct
  source 1..24: mol_type = other DNA
gccgccggctggaacttcgactac

SEQ ID NO: 91 - DNA, 18 bp, synthetic construct
  source 1..18: mol_type = other DNA
caggatgtgggcaccgct

SEQ ID NO: 92 - skipped

SEQ ID NO: 93 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 94 - DNA, 345 bp, synthetic construct
  source 1..345: mol_type = other DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 95 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 96 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgtggacaccagcaccgatacagcctacatggaactgagcagcctgagaagcgaggacaccgccgtgtattattgtgccaccggctggaacttcgactactggggacagggcacactggtcaccgttagctct

SEQ ID NO: 97 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaagagcaccgatacagcctacatggaactgagcagcctgagaagcgaggacaccgccgtgtattattgtgccaccggctggaacttcgactactggggacagggcacactggtcaccgttagctct

SEQ ID NO: 98 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccagcacagcctacatggaactgagcagcctgagaagcgaggacaccgccgtgtattattgtgccaccggctggaacttcgactactggggacagggcacactggtcaccgttagctct

SEQ ID NO: 99 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctct

SEQ ID NO: 100 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctct

SEQ ID NO: 101 - DNA, 345 bp, synthetic construct
  source 1..345: mol_type = other DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaagagcaccagcacagcctacatggaactgagcagcctgagaagcgaggacaccgccgtgtattattgtgccgccggctggaacttcgactactggggacagggcacactggtcaccgttagctct

SEQ ID NO: 102 - DNA, 345 bp, synthetic construct
  source 1..345: mol_type = other DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctct

SEQ ID NO: 103 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 104 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 105 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaacgaaaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 106 - DNA, 345 bp, synthetic construct
  source 1..345: mol_type = other DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacgccgaaaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctct

SEQ ID NO: 107 - DNA, 321 bp, synthetic construct
  source 1..321: mol_type = other DNA
gatattcagatgacacagagccctagcagcctgagcgcctctgtgggcgatagagtgacaatcacatgcaaggccagccaggatgtgggcaccgctgttgactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 108 - DNA, 321 bp, synthetic construct
  source 1..321: mol_type = other DNA
gatattcagatgacacagagccctagcagcctgagcgcctctgtgggcgatagagtgacaatcacatgcagagccagccaggatgtgggcaccgctgttgactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 109 - DNA, 321 bp, synthetic construct
  source 1..321: mol_type = other DNA
gatattcagatgacacagagccctagcagcctgagcgcctctgtgggcgatagagtgacaatcacatgcagagccagccaggatgtgggcaccgctctgaactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 110 - DNA, 321 bp, synthetic construct
  source 1..321: mol_type = other DNA
gatattcagatgacacagagccctagcagcctgagcgcctctgtgggcgatagagtgacaatcacatgcaaggccagccaggatgtgggcaccgctgttgactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 111 - DNA, 321 bp, synthetic construct
  source 1..321: mol_type = other DNA
gatattcagatgacacagagccctagcagcctgagcgcctctgtgggcgatagagtgacaatcacatgcagagccagccaggatgtgggcaccgctgttgactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 112 - DNA, 321 bp, synthetic construct
  source 1..321: mol_type = other DNA
gatattcagatgacacagagccctagcagcctgagcgcctctgtgggcgatagagtgacaatcacatgcagagccagccaggatgtgggcaccgctctgaactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaa

SEQ ID NO: 113 - DNA, 345 bp, synthetic construct
  source 1..345: mol_type = other DNA
gaagttcaacttgtgcagagcggccctgaagtgaagaaacctggcgctaccgtgaagatcagctgcaagaccagcggctacaccttcaccgagtacaccatccactgggttaagcaggcccctggcaaaggcctggaatggatcggcaacatcaaccccaacaacggcggcaccacctacaaccagaagttcgaggataaggccacactgaccgtggacaagagcaccgacacagcctacatggaactgagcagcctgagaagcgaggacaccgccgtgtattattgtgccgccggatggaacttcgactactggggacagggcaccctgctgacagtctcttct

SEQ ID NO: 114 - DNA, 321 bp, synthetic construct
  source 1..321: mol_type = other DNA
gatattcagatgacacagagccccagcagcctgagcacatctgtgggagatagagtgaccctgacctgcaaggcctctcaggatgtgggaaccgccgtggactggtatcagcagaaacccggaccttctccaaagctgctgatctactgggccagcaccagacacacaggcatccctagcagattttctggcagcggctctggcaccgacttcaccctgaccatttctagcctccagcctgaggacttcgccgactactactgccagcagtacaacagctaccctctgacattcggccctggcaccaaggtggacatcaaa

SEQ ID NO: 115 - protein, 445 aa, Homo sapiens
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 214 aa, Homo sapiens
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 118 - DNA, 1338 bp, Homo sapiens
  source 1..1338: mol_type = genomic DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 119 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 120 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgtggacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 121 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaagagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 122 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccagcacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 123 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 124 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 125 - DNA, 1338 bp, Homo sapiens
  source 1..1338: mol_type = genomic DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaagagcaccagcacagcctacatggaactgagcagcctgagaagcgaggacaccgccgtgtattattgtgccgccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 126 - DNA, 1338 bp, Homo sapiens
  source 1..1338: mol_type = genomic DNA
gaagttcaacttgttcagagcggagccgaagtgaagaaacccggcgctacagtgaagatcagctgcaagacctccggctacaccttcaccgagtacaccattcactgggtgcagcaggcccctggcaaaggactggaatggatcggcaacatcaaccccaacaacggcggcaccacctacaaccagaaattcgaggacagagtgaccatcaccgccgacaccagcaccgatacagcctacatggaactgagcagcctgagaagcgaggacaccgccgtgtattattgtgccgccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 127 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattcgagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 128 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaaccagaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 129 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacaacgaaaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 130 - DNA, 1338 bp, Homo sapiens
  source 1..1338: mol_type = genomic DNA
gaagttcaacttgttcagagcggagccgaagtgaagaaacccggcgctacagtgaagatcagctgcaaggtgtccggctacaccttcaccgagtacaccattcactgggtgcagcaggcccctggcaaaggactggaatggatgggcaacatcaaccccaacaacggcggcaccacctacgccgaaaaattccagggcagagtgaccatcaccgccgacaccagcaccgatacagcctacatggaactgagcagcctgagaagcgaggacaccgccgtgtattattgtgccaccggctggaacttcgactactggggacagggcacactggtcaccgttagctctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 131 - DNA, 645 bp, Homo sapiens
  source 1..645: mol_type = genomic DNA
gatattcagatgacacagagccctagcagcctgagcgcctctgtgggcgatagagtgacaatcacatgcaaggccagccaggatgtgggcaccgctgttgactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 132 - DNA, 645 bp, Homo sapiens
  source 1..645: mol_type = genomic DNA
gatattcagatgacacagagccctagcagcctgagcgcctctgtgggcgatagagtgacaatcacatgcagagccagccaggatgtgggcaccgctgttgactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 133 - DNA, 645 bp, Homo sapiens
  source 1..645: mol_type = genomic DNA
gatattcagatgacacagagccctagcagcctgagcgcctctgtgggcgatagagtgacaatcacatgcagagccagccaggatgtgggcaccgctctgaactggtatcagcagaagcctggcaaggcccctaagctgctgatctactgggccagcacaagacacacaggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 134 - DNA, 645 bp, Homo sapiens
  source 1..645: mol_type = genomic DNA
gatattcagatgacacagagccctagcagcctgagcgcctctgtgggcgatagagtgacaatcacatgcaaggccagccaggatgtgggcaccgctgttgactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 135 - DNA, 645 bp, Homo sapiens
  source 1..645: mol_type = genomic DNA
gatattcagatgacacagagccctagcagcctgagcgcctctgtgggcgatagagtgacaatcacatgcagagccagccaggatgtgggcaccgctgttgactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 136 - DNA, 645 bp, Homo sapiens
  source 1..645: mol_type = genomic DNA
gatattcagatgacacagagccctagcagcctgagcgcctctgtgggcgatagagtgacaatcacatgcagagccagccaggatgtgggcaccgctctgaactggtatcagcagaagcctggcaaggcccctaagctgctgatctactgggccagcacaagacacagcggcgtgccaagcagattttctggcagcggctctggcaccgacttcaccctgaccatttctagcctccagcctgaggacttcgccacctactactgtcagcagtacaacagctaccctctgacctttggccagggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 137 - DNA, 1338 bp, Homo sapiens
  source 1..1338: mol_type = genomic DNA
gaagttcaacttgtgcagagcggccctgaagtgaagaaacctggcgctaccgtgaagatcagctgcaagaccagcggctacaccttcaccgagtacaccatccactgggttaagcaggcccctggcaaaggcctggaatggatcggcaacatcaaccccaacaacggcggcaccacctacaaccagaagttcgaggataaggccacactgaccgtggacaagagcaccgacacagcctacatggaactgagcagcctgagaagcgaggacaccgccgtgtattattgtgccgccggatggaacttcgactactggggacagggcaccctgctgacagtctcttctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 138 - DNA, 645 bp, Homo sapiens
  source 1..645: mol_type = genomic DNA
gatattcagatgacacagagccccagcagcctgagcacatctgtgggagatagagtgaccctgacctgcaaggcctctcaggatgtgggaaccgccgtggactggtatcagcagaaacccggaccttctccaaagctgctgatctactgggccagcaccagacacacaggcatccctagcagattttctggcagcggctctggcaccgacttcaccctgaccatttctagcctccagcctgaggacttcgccgactactactgccagcagtacaacagctaccctctgacattcggccctggcaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 139 - DNA, 345 bp, synthetic construct
  source 1..345: mol_type = other DNA
gaagttcaacttcagcagtctggccccgagctgaagaaacctggaacaagcgtgcggatcagctgcaagaccagcggctacaccttcaccgagtacaccatccactgggtcaagcagagccacggcaagtctctggaatggatcggcaacatcaaccccaacaacggcggcaccacctacaaccagaagttcgaggataaggccacactgaccgtggacaagagcagcagcacagcctacatggaactgagaagcctgaccagcgaggacagcgccgtgtattattgtgccgccggatggaacttcgactactggggacagggcacaaccctgaccgttagctct

SEQ ID NO: 140 - DNA, 321 bp, synthetic construct
  source 1..321: mol_type = other DNA
gatattgtgatgacccagagccacaagttcatgagcaccagcgtgggcgacagagtgtccatcatctgcaaggcctctcaggatgtgggcaccgccgttgactggtatcagcagaaacctggacagagccccaagctgctgatctactgggcctctacaagacacacaggcgtgcccgatagattcacaggctctggcagcggcaccgactttaccctgaccatcacaaacgtgcagagcgaggacctggccgactacttctgccagcagtacaacagctaccctctgacctttggagccggcaccatgctggatcttaaa

SEQ ID NO: 141 - protein, 4 aa, synthetic construct
GGFG

SEQ ID NO: 142 - protein, 4 aa, synthetic construct
GFLG

SEQ ID NO: 143 - protein, 4 aa, synthetic construct
ALAL

SEQ ID NO: 144 - protein, 4 aa, synthetic construct
  SITE 1: note = 6-(2,5-dioxo-2,5-dihydro-1H-pyrrol-1-yl)hexanoyl modified residue
  SITE 4: note = (4-{[(2S)-2-{[(1R,3R,15E,28R,29R,30R,31R,34S,36R,39 R,41R)-29,41-difluoro-34,39-dioxo-34,39-disulfanyl-2,33,35 ,38,40,42-hexaoxa-4,6,9,11,13,18,20,22,25,27-decaaza-34lam bda5,39lambda5-diphosphaoctacyclo[28.6.4.13,36.128,31.04,8 .07,12.019,24.023,27]dotetraconta-5,7,9,11,15,19,21,23,25- nonaene-13-carbonyloxy]methyl}pyrrolidine-1-carbonyloxy]me thyl}phenyl)amino modified residue
GGFG

(19) United States
(12) Patent Application Publication
(10) Pub. No.: US 2026/0241024 A1
(43) Pub. Date: Aug. 20, 2026

(54) ANTI-PSMA ANTIBODIES, CONJUGATES, AND METHODS OF USE

(71) Applicant: EISAI R&D MANAGEMENT CO., LTD., Tokyo (JP)

(72) Inventors: Jared SPIDEL, Downingtown, PA (US); Dae-Shik KIM, Andover, MA (US); Kenzo ARAI, Tsukuba-shi, Ibaraki (JP); Keiji FURUUCHI, Wynnewood, PA (US); Xin CHENG, Wallingford, PA (US); Xingfeng BAO, Concord, MA (US); Yoshinobu YAMANE, Tsukuba-shi, Ibaraki (JP); Yosuke KABURAGI, Tsukuba-shi, Ibaraki (JP); Kuan-Chun HUANG, Lexington, MA (US); Earl F. ALBONE, Collegeville, PA (US)

(21) Appl. No.: 19/160,408

(22) PCT Filed: Feb. 28, 2024
(86) PCT No.: PCT/US2024/017767

§ 371 Date: Aug. 27, 2025

Related U.S. Application Data
(60) Provisional application No. 63/487,553, filed on Feb. 28, 2023.
(60) Provisional application No. 63/557,342, filed on Feb. 23, 2024.

(51) Int. Cl. A61K 47/68; A61P 35/00; C07K 16/30

(52) U.S. Cl. CPC A61K 47/6807; A61K 47/6869; A61K 47/6889; A61P 35/00; C07K 16/3069; C07K 2317/24; C07K 2317/33; C07K 2317/52; C07K 2317/55; C07K 2317/565; C07K 2317/94

(57) ABSTRACT

Antibodies, antigen-binding fragments, and conjugates (e.g., antibody-drug conjugates) thereof that bind PSMA, as well as STING agonist linker-drug conjugates and preparation thereof, are disclosed. The disclosure further relates to methods and compositions for use in the treatment of, e.g., cancer by administering the compositions provided herein.

This application claims the benefit of U.S. Provisional Application No. 63/487,553, filed on Feb. 28, 2023, and U.S. Provisional Application No. 63/557,342, filed on Feb. 23, 2024, the contents of which are incorporated by reference in their entirety.

The present disclosure relates to anti-PSMA antibodies and antigen-binding fragments thereof, as well as conjugates such as antibody drug conjugates (ADCs), e.g., those comprising a STING agonist, and their use in the treatment and diagnosis of cancers that express PSMA and/or are amenable to treatment by modulating STING pathway activity or by administering a composition disclosed herein.

Prostate cancer is the second most common type of cancer and the second leading cause of cancer death in men. There are currently limited treatment options for metastatic prostate cancer, with poor prognosis in such cases and a need to develop treatments with greater efficacy.

Prostate-specific membrane antigen (PSMA) is a cell-surface antigen that is highly expressed in prostate cancer. Expression levels of PSMA increase along with prostate cancer progression, with high expression of PSMA maintained at metastatic sites. Anti-PSMA antibodies have previously been generated, including modified antibodies with reduced immunogenicity in humans. See, e.g., U.S. Pat. Nos. 7,045,605 and 11,059,903. Examples of antibodies that bind PSMA are J591 and deimmunized J591 (deJ591). The amino acid sequence of the heavy chain variable domain of the deJ591 antibody is given herein as SEQ ID NO: 40 and the corresponding light chain variable domain is given herein as SEQ ID NO: 41. However, clinical trials using this antibody have shown undesirable effects of immunogenicity, including myelosuppression and liver enzyme abnormalities. See, e.g., de Bono et al. (2021) Clin Cancer Res 27 (13): 3602-3609. There remains a need for improved PSMA antibodies, e.g., those that are fully humanized to minimize immunogenicity while retaining desirable properties such as good target binding affinity, low off-target binding, and good stability.

Given the high expression of PSMA in prostate cancer, it may be used as a target for tumor antigen-specific drug delivery approaches, e.g., an antibody-mediated approach. Antibodies conjugated with cytotoxic compounds such as chemotherapeutics have also been explored to enhance the cell-killing activity of antibody-based drug delivery to tumor cells. Nevertheless, the need remains to provide suitable antibodies and/or ADCs, such as those that offer a combination of efficient prostate tumor targeting, on-target effects, and/or reduced off-target effects.

STING (stimulator of interferon genes) is a pattern-recognition receptor that senses cyclic dinucleotides in the cytosol and induces the expression of type I interferons and other inflammatory cytokines (e.g., interferon-β (IFN-β), tumor necrosis factor alpha (TNFα), C-X-C Motif Chemokine Ligand 10 (CXCL10), interleukin-6 (IL-6)), which in turn mediate the innate immune response to infections or diseases, e.g., cancer. STING signaling has been shown to have antitumor effects such as modulation of the vasculature and augmentation of adaptive immunity. First-generation STING agonists, e.g., cyclic dinucleotides, often require intra-tumoral injection and show only modest systemic efficacy. These STING agonists are also poorly membrane permeable, which may limit their ability to engage STING inside the cell.

While uses of STING agonists for treating infection or disease have been reported in the art, there remains an unmet need for delivery systems that would allow for systemic administration of STING agonists that specifically target tumor sites. Likewise, there remains a need in the art for improved antibodies that bind PSMA with superior properties, e.g., with respect to antigen-binding and/or the ability to effectively deliver payloads such as a STING agonist to a target cell or tissue expressing PSMA.

### SUMMARY OF THE INVENTION

In various embodiments, the present disclosure provides, in part, novel antibodies and antigen-binding fragments that are capable of specifically binding PSMA and may be used alone or linked to one or more additional agents (e.g., as ADCs) and administered as part of pharmaceutical compositions. In some embodiments, the antibodies, antigen-binding fragments, and/or ADCs of the present disclosure may be used to slow, inhibit, and/or reverse tumor growth in mammals, and may be useful for treating human cancer patients.

The present disclosure more specifically relates, in various embodiments, to antibodies and antibody-drug conjugate compounds that are capable of binding and/or killing PSMA-expressing cells. In various embodiments, the compounds are also capable of internalizing into a target PSMA-expressing cell after binding. Anti-PSMA-ADC compounds comprising a linker that attaches a STING agonist moiety, e.g., Formula (III), Formula (IV), or a compound of Table 14, e.g., Compound 1, to an anti-PSMA antibody moiety are disclosed. An anti-PSMA antibody moiety may be a full-length antibody or antigen-binding fragment.

In various embodiments, the present disclosure provides a humanized anti-prostate-specific membrane antigen (PSMA) antibody or antigen-binding fragment thereof, wherein the antibody or antigen-binding fragment binds specifically to human PSMA, and wherein the antibody or antigen-binding fragment comprises (i) three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or (ii) three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 33 (LCDR1), SEQ ID NO: 36 (LCDR2), and SEQ ID NO: 37 (LCDR3), as defined by the Kabat numbering system; or (iii) three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

In some embodiments, the anti-PSMA antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

In some embodiments, the anti-PSMA antibody or antigen-binding fragment comprises a heavy chain variable region comprising an amino acid sequence of SEQ ID NO: 1, and a light chain variable region comprising an amino acid sequence of SEQ ID NO: 15. In some embodiments, the anti-PSMA antibody or antigen-binding fragment comprises a heavy chain variable region comprising an amino acid sequence of SEQ ID NO: 2, and a light chain variable region comprising an amino acid sequence of SEQ ID NO: 15. In some embodiments, the anti-PSMA antibody or antigen-binding fragment comprises a heavy chain variable region comprising an amino acid sequence of SEQ ID NO: 3, and a light chain variable region comprising an amino acid sequence of SEQ ID NO: 15. In some embodiments, the anti-PSMA antibody or antigen-binding fragment comprises a heavy chain variable region comprising an amino acid sequence of SEQ ID NO: 14, and a light chain variable region comprising an amino acid sequence of SEQ ID NO: 15. In some embodiments, the anti-PSMA antibody or antigen-binding fragment comprises a heavy chain variable region comprising an amino acid sequence of SEQ ID NO: 14, and a light chain variable region comprising an amino acid sequence of SEQ ID NO: 19.

In some embodiments, the anti-PSMA antibody or antigen-binding fragment comprises a human IgG1 heavy chain constant region. In some embodiments, the anti-PSMA antibody or antigen-binding fragment comprises a human Ig kappa light chain constant region.

In some embodiments, the anti-PSMA antigen-binding fragment has a melting temperature (Tm)>80° C. In some embodiments, the antigen-binding fragment is a Fab.

In some embodiments, the anti-PSMA antibody or antigen-binding fragment is attached to at least one linker. In some embodiments, the at least one linker is cleavable. In some embodiments, the at least one linker is conjugated to a cytotoxic agent or detectable reagent.

In various embodiments, the present disclosure also provides, in part, novel linker-payload conjugates. The present disclosure more specifically relates, in various embodiments, to linker-payload conjugates comprising a linker that attaches a STING agonist moiety, e.g., Formula (III), Formula (IV), or a compound of Table 14, e.g., Compound 1, to an anti-PSMA antibody moiety.

In various embodiments, the present disclosure provides a linker-payload conjugate comprising L-D, wherein L is a linker that covalently attaches to D, wherein D comprises a compound according to one of the following Formulae:

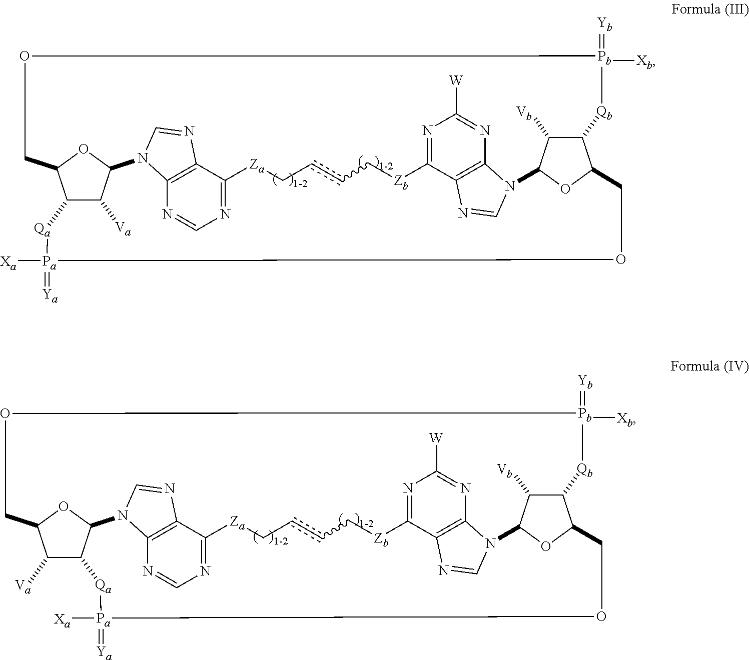

an isomer thereof, a deuterated derivative of the compound or isomer; or a salt of the compound, isomer, or deuterated derivative;
wherein, independently for each occurrence,
each of Pₐ and P_{b}, when not racemic, is independently selected from (R)-stereochemistry and (S)-stereochemistry;
each of Qₐ and Q_{b} is independently selected from NH and O;
each of Vₐ and V_{b} is independently selected from F and OH;
W is selected from H and NH₂;
each of Xₐ and X_{b} is independently selected from OH and SH;
each of Yₐ and Y_{b} is independently selected from O and S;
each Zₐ and Z_{b} is independently selected from CH₂, O, and NH; and
[symbol] Means that the bond is selected from a single bond ( [symbol] ), a double bond ( [symbol] ) of (E)- or (Z)-configuration, or a triple bond ( [symbol] );
   provided that at least one of Zₐ and Z_{b} is NH or at least one of Xₐ and X_{b} is SH.

In some embodiments, Pₐ is (S)-configuration and P_{b} is (R)-configuration. In some embodiments, Pₐ is (R)-configuration and P_{b} is (R)-configuration. In some embodiments, Qₐ and Q_{b} are O. In some embodiments, Vₐ and V_{b} are OH. In some embodiments, Vₐ and V_{b} are F. In some embodiments, W is H. In some embodiments, at least one of Zₐ and Z_{b} is NH. In some embodiments, Zₐ and Z_{b} are NH. In some embodiments, [symbol] comprises a double bond ( [symbol] ) of (E)- or (Z)-configuration. In some embodiments, the bridge

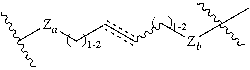

has the structure

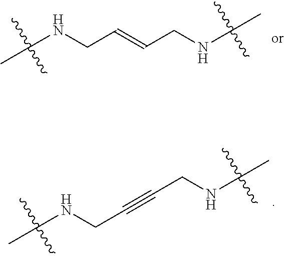 [SMILES: *NC/C=C/CN* ; *NCC#CCN*]

In some embodiments, at least one of Yₐ and Y_{b} is O. In some embodiments, Yₐ and Y_{b} are O. In some embodiments, at least one of Xₐ and X_{b} is SH. In some embodiments, Xₐ and X_{b} are SH. In some embodiments, D comprises a compound of Formula (III).

In some embodiments, D comprises a compound of Formula (III) selected from:

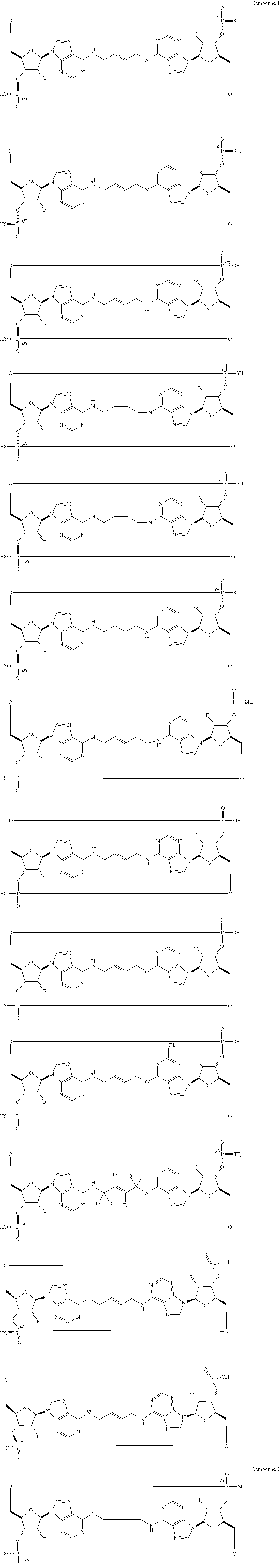

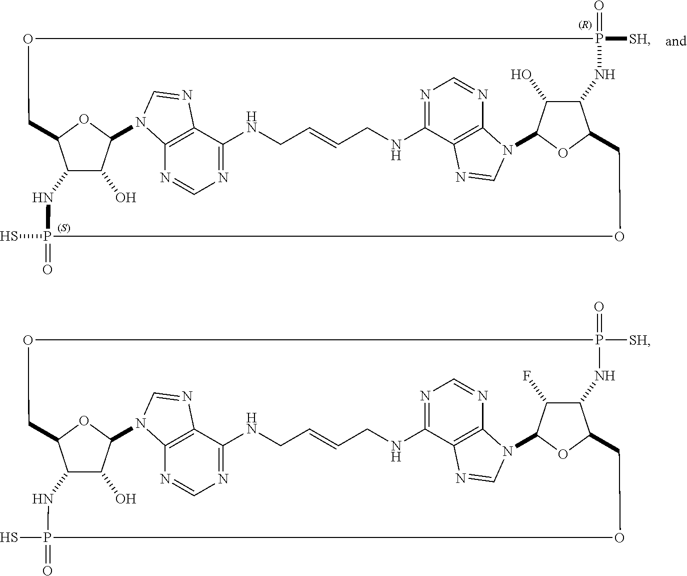

and salts thereof.

In some embodiments, D comprises a compound of Formula (III) selected from:

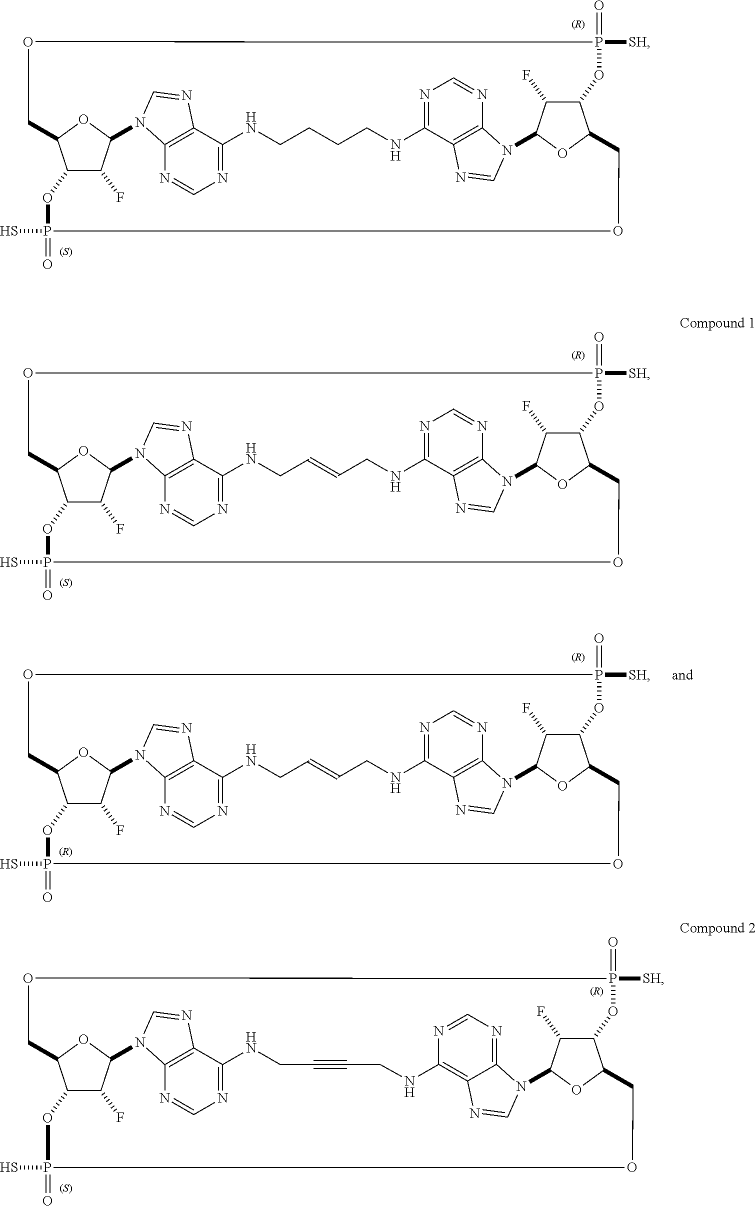

and salts thereof.

In some embodiments, D comprises Compound 1.

In some embodiments, D comprises Compound 2.

In some embodiments, at least one of Xₐ and X_{b} is SH and L is attached to D via a sulfur atom at the S-2 sulfur or the S-14 sulfur. In some embodiments, X_{b} is SH and L is attached to D at the S-2 sulfur. In some embodiments, Xₐ is SH and L is attached to D at the S-14 sulfur.

In some embodiments, at least one of Zₐ and Z_{b} is NH and L is attached to D via a nitrogen atom at the N-34 nitrogen or the N-39 nitrogen. In some embodiments, Z_{b} is NH and L is attached to D at the N-34 nitrogen. In some embodiments, Zₐ is NH and L is attached to D at the N-39 nitrogen.

In some embodiments, L is a cleavable linker. In some embodiments, the cleavable linker comprises a cleavable peptide moiety. In some embodiments, the cleavable peptide moiety is cleavable by a protease, optionally wherein the protease is a cathepsin or a legumain. In some embodiments, the cleavable peptide moiety comprises an amino acid unit. In some embodiments, the amino acid unit comprises Val-Ala, Val-Cit, Val-Lys, Ala-Ala-Asn, Ala-(NMe)Ala-Asn, Asn, Gly-Gly-Phe-Gly (SEQ ID NO: 141), Glu-Val-Ala, or Gly-Val-Ala. In some embodiments, the cleavable linker comprises Val-Cit. In some embodiments, the cleavable linker comprises Val-Ala.

In some embodiments, the linker comprises a maleimide (Mal) moiety. In some embodiments, the Mal moiety comprises maleimidocaproyl (MC). In some embodiments, the Mal moiety is joined to an antibody or antigen-binding fragment via a cysteine residue on the antibody or antigen-binding fragment.

In some embodiments, the linker further comprises at least one spacer unit. In some embodiments, the at least one spacer unit comprises at least one polyethylene glycol (PEG) moiety. In some embodiments, the at least one PEG moiety comprises -(PEG)ₘ- and m is an integer from 1 to 10. In some embodiments, m is an integer from 2 to 8. In some embodiments, m is an integer from 2 to 5. In some embodiments, m is 2. In some embodiments, the at least one spacer unit comprises PEG₂-Lys(ε-PEG₈-OMe)-PEG₂.

In some embodiments, the at least one spacer unit comprises.

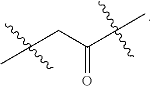 [SMILES: *CC(*)=O]

In some embodiments, the at least one spacer unit comprises

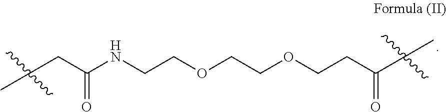 [SMILES: *CC(=O)NCCOCCOCCC(*)=O]

In some embodiments, the linker further comprises at least one self-immolative unit. In some embodiments, the linker comprises a first self-immolative unit. In some embodiments, the linker is capable of being removed from D after cleavage of the linker by self-immolation of the first self-immolative unit. In some embodiments, the first self-immolative unit comprises a p-aminobenzyl (pAB) optionally substituted with 1-3 substituents chosen from methyl, fluoro, chloro, trifluoromethyl, aryl, and heteroaryl. In some embodiments, the first self-immolative unit comprises a p-aminobenzyl (pAB). In some embodiments, the linker comprises MC-Val-Ala-pAB.

In some embodiments, the first self-immolative unit comprises a p-aminobenzyloxycarbonyl (pABC).

In some embodiments, the linker further comprises a second self-immolative unit. In some embodiments, the linker is capable of being removed from D after cleavage of the linker by self-immolation of the first self-immolative unit and/or self-immolation of the second self-immolative unit. In some embodiments, the linker is removed from D after cleavage of the linker in a stepwise fashion by self-immolation of the first self-immolative unit and then self-immolation of the second self-immolative unit.

In some embodiments, the linker comprises a cleavable linker, a first self-immolative unit, and a second self-immolative unit. In some embodiments, the cleavable linker comprises Val-Ala. In some embodiments, the cleavable linker comprises Val-Cit. In some embodiments, the cleavable linker comprises Formula (II).

In some embodiments, the second self-immolative unit comprises one of the following moieties:

| Second Self- | |
|---|---|
| immolative Unit | Chemical Structure |
| Unit 1 (MEC) | 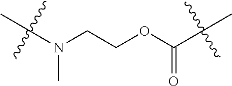 [SMILES: *C(=O)OCCN(*)C] |
| Unit 2 | 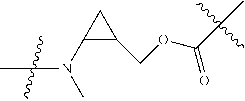 [SMILES: *C(=O)OCC1CC1N(*)C] |
| Unit 3 | 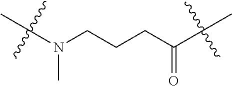 [SMILES: *C(=O)CCCN(*)C] |
| Unit 4 | 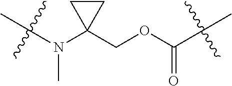 [SMILES: *C(=O)OCC1(N(*)C)CC1] |
| Unit 5 | 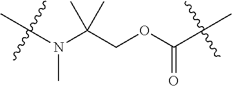 [SMILES: *C(=O)OCC(C)(C)N(*)C] |
| Unit 6 | 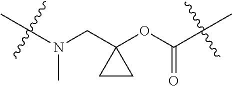 [SMILES: *C(=O)OC1(CN(*)C)CC1] |
| Unit 7 | 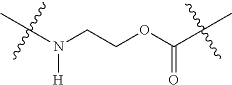 [SMILES: *NCCOC(*)=O] |
| Unit 8 | 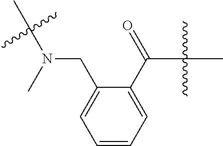 [SMILES: *C(=O)c1ccccc1CN(*)C] |
| Unit 9 | 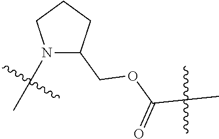 [SMILES: *C(=O)OCC1CCCN1*] |
| Unit 10 | 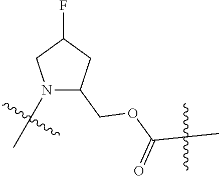 [SMILES: *C(=O)OCC1CC(F)CN1*] |
| Unit 11 | 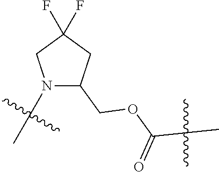 [SMILES: *C(=O)OCC1CC(F)(F)CN1*] |
| Unit 12 | 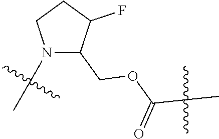 [SMILES: *C(=O)OCC1C(F)CCN1*] |
| Unit 13 | 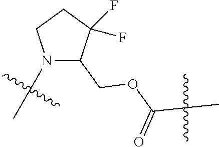 [SMILES: *C(=O)OCC1N(*)CCC1(F)F] |

or an isomer thereof.

In some embodiments, the cleavable linker comprises Val-Ala and wherein the second self-immolative unit comprises one of the following moieties:

| Second Self- | |
|---|---|
| immolative Unit | Chemical Structure |
| Unit 1 (MEC) | 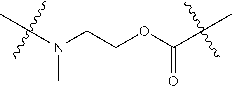 [SMILES: *C(=O)OCCN(*)C] |
| Unit 2 | 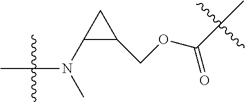 [SMILES: *C(=O)OCC1CC1N(*)C] |
| Unit 3 | 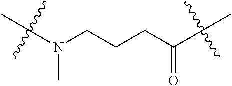 [SMILES: *C(=O)CCCN(*)C] |
| Unit 4 | 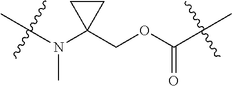 [SMILES: *C(=O)OCC1(N(*)C)CC1] |
| Unit 5 | 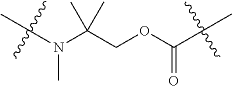 [SMILES: *C(=O)OCC(C)(C)N(*)C] |
| Unit 6 | 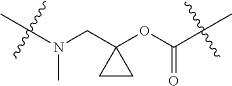 [SMILES: *C(=O)OC1(CN(*)C)CC1] |
| Unit 7 | 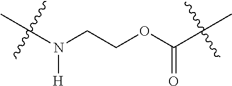 [SMILES: *NCCOC(*)=O] |
| Unit 8 | 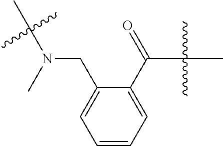 [SMILES: *C(=O)c1ccccc1CN(*)C] |
| Unit 9 | 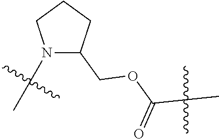 [SMILES: *C(=O)OCC1CCCN1*] |
| Unit 10 | 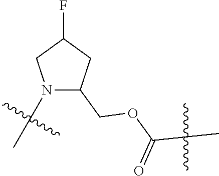 [SMILES: *C(=O)OCC1CC(F)CN1*] |
| Unit 11 | 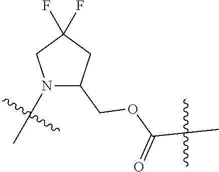 [SMILES: *C(=O)OCC1CC(F)(F)CN1*] |
| Unit 12 | 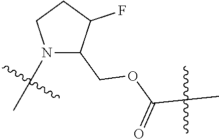 [SMILES: *C(=O)OCC1C(F)CCN1*] |
| Unit 13 | 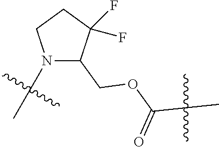 [SMILES: *C(=O)OCC1N(*)CCC1(F)F] |

or an isomer thereof.

In some embodiments, the second self-immolative unit comprises a Unit 1 (MEC) moiety. In some embodiments, the second self-immolative unit comprises a Unit 8 moiety. In some embodiments, the second self-immolative unit comprises a Unit 11 moiety. In some embodiments, the second self-immolative unit comprises a Unit 9 moiety.

In some embodiments, the linker comprises Val-Ala-pABC-MEC moiety. In some embodiments, the linker comprises MC-Val-Ala-pABC-MEC moiety.

In some embodiments, the L-D comprises LP1:

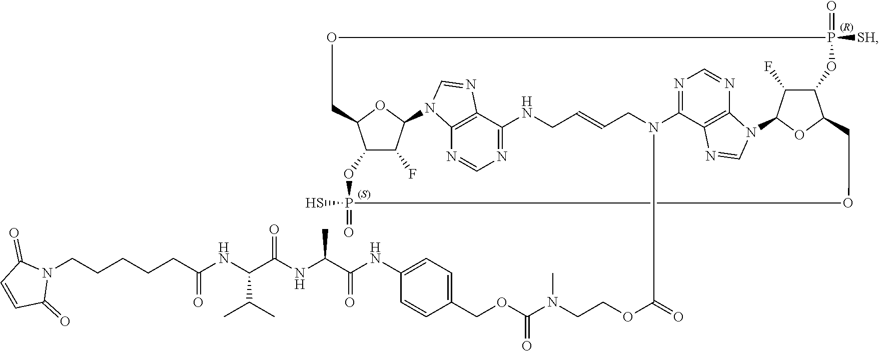

### or LP2:

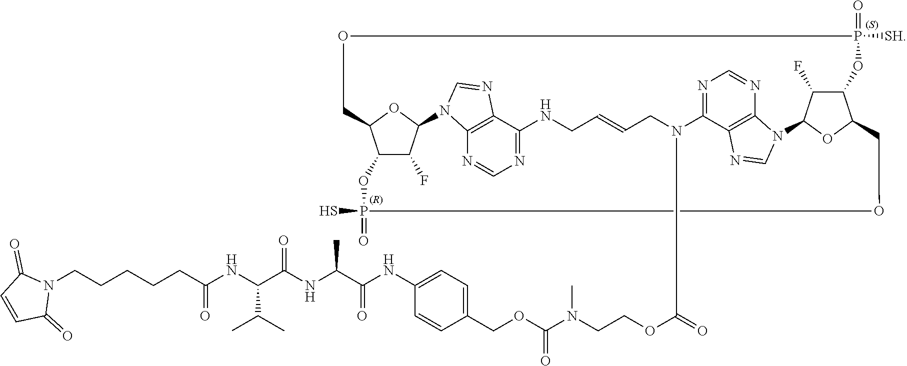

In some embodiments, the linker comprises Val-Cit-pABC-MEC moiety. In some embodiments, the linker comprises MC-Val-Cit-pABC-MEC moiety. In some embodiments, MC-Val-Cit-pABC-MEC-Compound 1.

In some embodiments, the linker comprises Val-Ala-pABC-Unit 8 moiety. In some embodiments, the linker comprises MC-Val-Ala-pABC-Unit 8 moiety.

In some embodiments, the L-D comprises LP16:

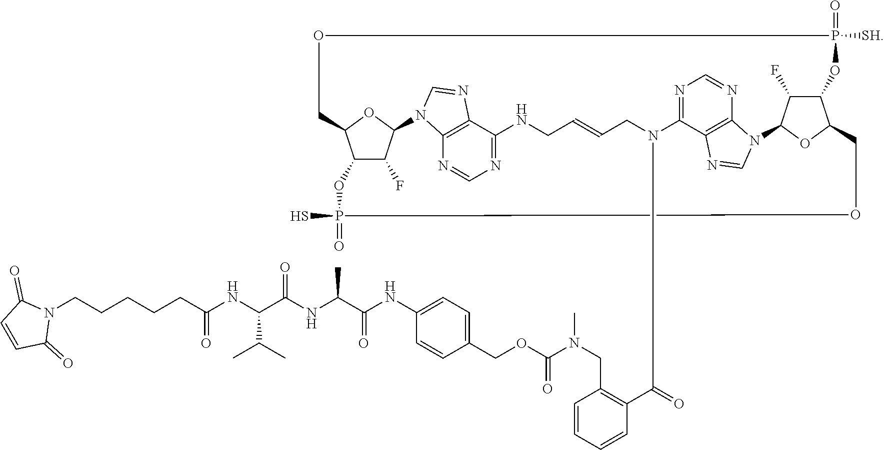 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Val-Cit-pABC-Unit 8 moiety. In some embodiments, the linker comprises MC-Val-Cit-pABC-Unit 8 moiety. In some embodiments, the L-D comprises MC-Val-Cit-pABC-Unit 8-Compound 1.

In some embodiments, the linker comprises Val-Ala-pABC-Unit 11 moiety. In some embodiments, the linker comprises MC-Val-Ala-pABC-Unit 11 moiety.

In some embodiments, the L-D comprises LP28:

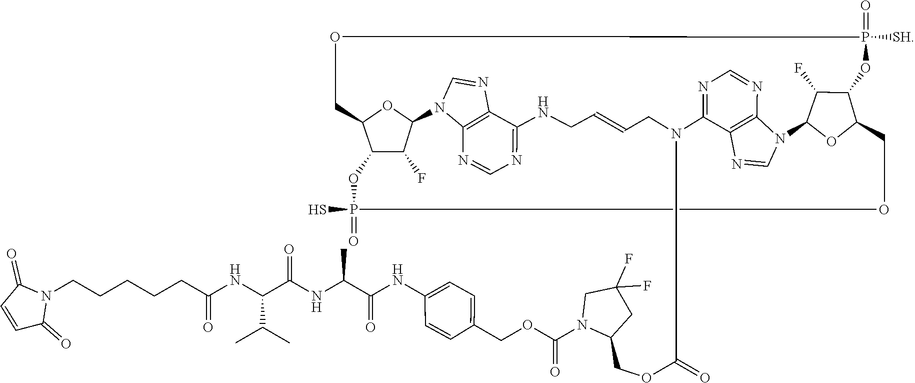 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Val-Cit-pABC-Unit 11 moiety. In some embodiments, the linker comprises MC-Val-Cit-pABC-Unit 11 moiety. In some embodiments, the L-D comprises MC-Val-Cit-pABC-Unit 11-Compound 1.

In some embodiments, the linker comprises Val-Ala-pABC-Unit 9 moiety. In some embodiments, the linker comprises MC-Val-Ala-pABC-Unit 9 moiety.

In some embodiments, the L-D comprises LP20:

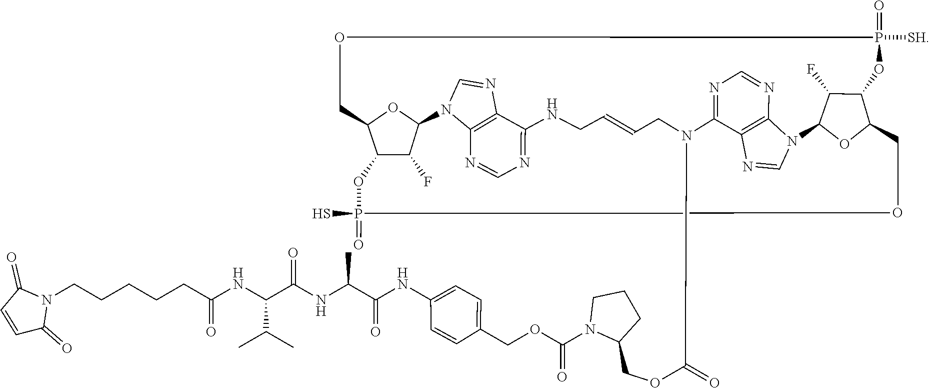 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Val-Cit-pABC-Unit 9 moiety. In some embodiments, the linker comprises MC-Val-Cit-pABC-Unit 9 moiety. In some embodiments, the L-D comprises MC-Val-Cit-pABC-Unit 9-Compound 1.

In some embodiments, the linker comprises Formula (II)-Val-Cit-pABC. In some embodiments, the linker comprises Formula (II)-Val-Cit-pABC-MEC moiety. In some embodiments, the linker comprises Mal-Formula (II)-Val-Cit-pABC-MEC moiety. In some embodiments, the L-D comprises Mal-Formula (II)-Val-Cit-pABC-MEC-Compound 1. In some embodiments, the linker comprises Formula (II)-Val-Cit-pABC-Unit 8 moiety. In some embodiments, the linker comprises Mal-Formula (II)-Val-Cit-pABC-Unit 8 moiety. In some embodiments, the L-D comprises Mal-Formula (II)-Val-Cit-pABC-Unit 8-Compound 1. In some embodiments, the linker comprises Formula (II)-Val-Cit-pABC-Unit 11 moiety. In some embodiments, the linker comprises Mal-Formula (II)-Val-Cit-pABC-Unit 11 moiety. In some embodiments, the L-D comprises Mal-Formula (II)-Val-Cit-pABC-Unit 11-Compound 1. In some embodiments, the linker comprises Formula (II)-Val-Cit-pABC-Unit 9 moiety. In some embodiments, the linker comprises Mal-Formula (II)-Val-Cit-pABC-Unit 9 moiety. In some embodiments, the L-D comprises Mal-Formula (II)-Val-Cit-pABC-Unit 9-Compound 1.

In some embodiments, the linker comprises Formula (II)-Val-Ala-pABC. In some embodiments, the linker comprises Formula (II)-Val-Ala-pABC-MEC moiety. In some embodiments, the linker comprises Mal-Formula (II)-Val-Ala-pABC-MEC moiety. In some embodiments, the L-D comprises Mal-Formula (II)-Val-Ala-pABC-MEC-Compound 1. In some embodiments, the linker comprises Formula (II)-Val-Ala-pABC-Unit 8 moiety. In some embodiments, the linker comprises Mal-Formula (II)-Val-Ala-pABC-Unit 8 moiety. In some embodiments, the L-D comprises Mal-Formula (II)-Val-Ala-pABC-Unit 8-Compound 1. In some embodiments, the linker comprises Formula (II)-Val-Ala-pABC-Unit 11 moiety. In some embodiments, the linker comprises Mal-Formula (II)-Val-Ala-pABC-Unit 11 moiety. In some embodiments, the L-D comprises Mal-Formula (II)-Val-Ala-pABC-Unit 11-Compound 1. In some embodiments, the linker comprises Formula (II)-Val-Ala-pABC-Unit 9 moiety. In some embodiments, the linker comprises Mal-Formula (II)-Val-Ala-pABC-Unit 9 moiety. In some embodiments, the L-D comprises Mal-Formula (II)-Val-Ala-pABC-Unit 9-Compound 1.

In some embodiments, the linker comprises Formula (II)-Val-Cit-pAB. In some embodiments, the linker comprises Formula (II)-Val-Cit-pAB-Unit 9 moiety. In some embodiments, the linker comprises Mal-Formula (II)-Val-Cit-pAB. In some embodiments, the linker comprises Mal-Formula (II)-Val-Cit-pAB-Unit 9 moiety. In some embodiments, the L-D comprises Mal-Formula (II)-Val-Cit-pAB-Unit 9-Compound 1.

In some embodiments, the linker comprises Formula (II)-Val-Ala-pAB. In some embodiments, the linker comprises Formula (II)-Val-Ala-pAB-Unit 9 moiety. In some embodiments, the linker comprises Mal-Formula (II)-Val-Ala-pAB. In some embodiments, the linker comprises Mal-Formula (II)-Val-Ala-pAB-Unit 9 moiety.

In some embodiments, the L-D comprises LP25:

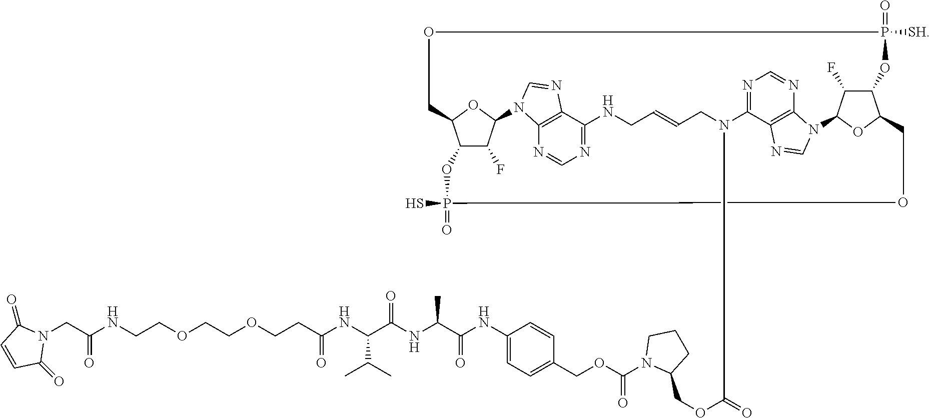 [SMILES: CC(C)[C@H](NC(=O)CCOCCOCCNC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the linker comprises Formula (II)-Val-Cit-pAB-Unit 11 moiety. In some embodiments, the linker comprises Mal-Formula (II)-Val-Cit-pAB-Unit 11 moiety. In some embodiments, the L-D comprises Mal-Formula (II)-Val-Cit-pAB-Unit 11-Compound 1.

In some embodiments, the linker comprises Formula (II)-Val-Ala-pAB-Unit 11 moiety. In some embodiments, the linker comprises Mal-Formula (II)-Val-Ala-pAB-Unit 11 moiety.

In some embodiments, the L-D comprises LP26:

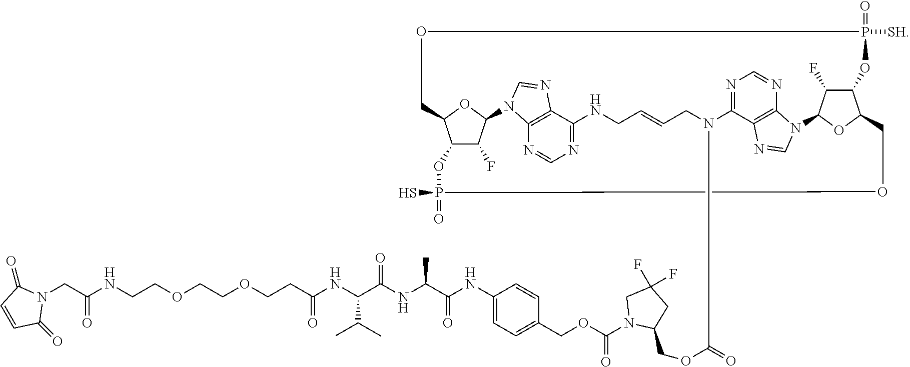

In various embodiments, the present disclosure provides an antibody-drug conjugate of Formula (I):

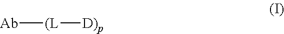

wherein Ab is an anti-PSMA antibody or antigen-binding fragment thereof disclosed herein; L-D is a linker-payload conjugate disclosed herein; and p is an integer from 1 to 20.

In some embodiments, p is an integer from 1 to 12. In some embodiments, p is an integer from 2 to 8. In some embodiments, p is an integer from 2 to 4. In some embodiments, p is 2. In some embodiments, p is 4.

In some embodiments, the cleavable linker comprises a cleavable moiety that is positioned such that no part of the linker or the antibody or antigen-binding fragment remains bound to D upon cleavage.

In some embodiments, the linker-payload conjugate attaches to the antibody or antigen-binding fragment via a Mal moiety. In some embodiments, the Mal moiety is joined to the antibody or antigen-binding fragment via a cysteine residue on the antibody or antigen-binding fragment. In some embodiments, the cysteine residue is on the light chain of the antibody or antigen-binding fragment. In some embodiments, the cysteine residue is on the heavy chain of the antibody or antigen-binding fragment.

In some embodiments, the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising amino acid sequences of SEQ ID NO: 32 (LCDR1), SEQ ID NO: 34 (LCDR2), and SEQ ID NO: 36 (LCDR3), as defined by the Kabat numbering system.

In some embodiments, the antibody or antigen-binding fragment comprises three HCDRs comprising amino acid sequences of SEQ ID NO: 28 (HCDR1), SEQ ID NO: 29 (HCDR2), and SEQ ID NO: 30 (HCDR3); and three LCDRs comprising amino acid sequences of SEQ ID NO: 37 (LCDR1), SEQ ID NO: 38 (LCDR2), and SEQ ID NO: 36 (LCDR3), as defined by the IMGT numbering system.

In some embodiments, the antibody or antigen-binding fragment comprises a heavy chain variable region comprising an amino acid sequence of SEQ ID NO: 14, and a light chain variable region comprising an amino acid sequence of SEQ ID NO: 19.

In some embodiments, L-D comprises LP16, LP20, LP26, or LP28.

In some embodiments, the L-D comprises LP16:

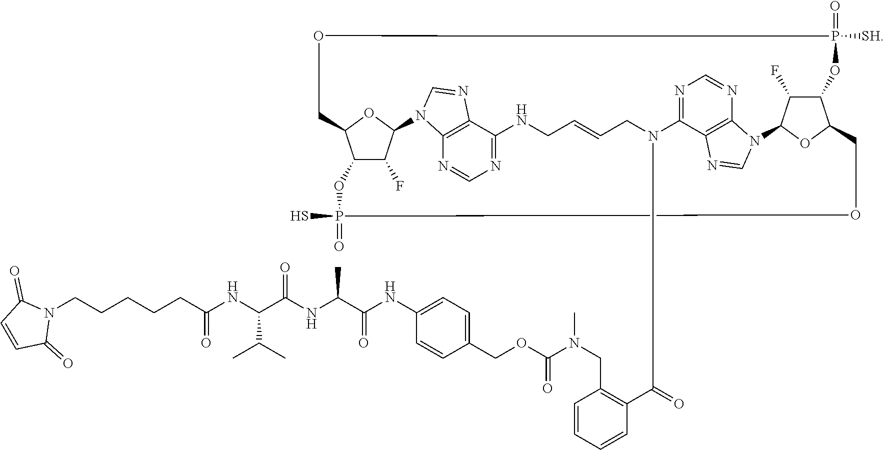 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the L-D comprises LP20:

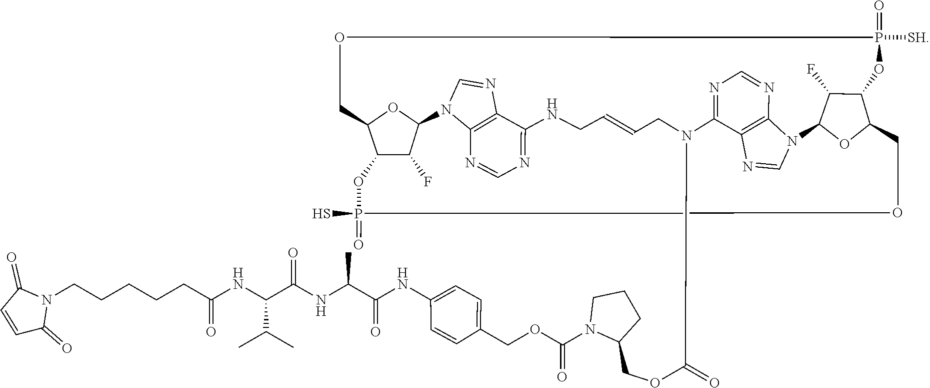 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the L-D comprises LP26:

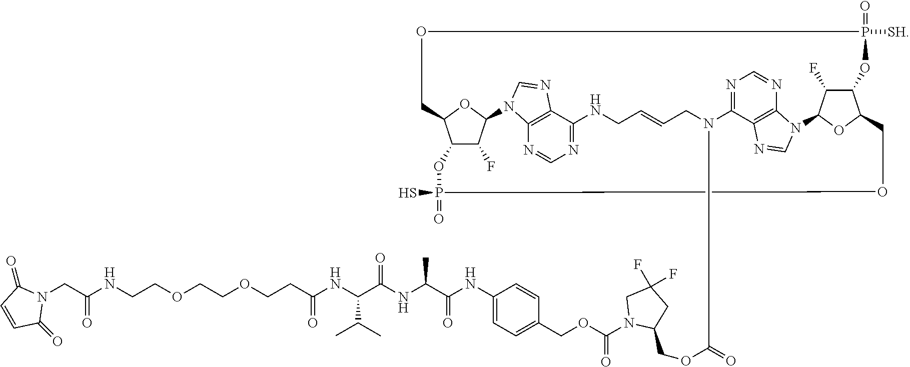

In some embodiments, the L-D comprises LP28:

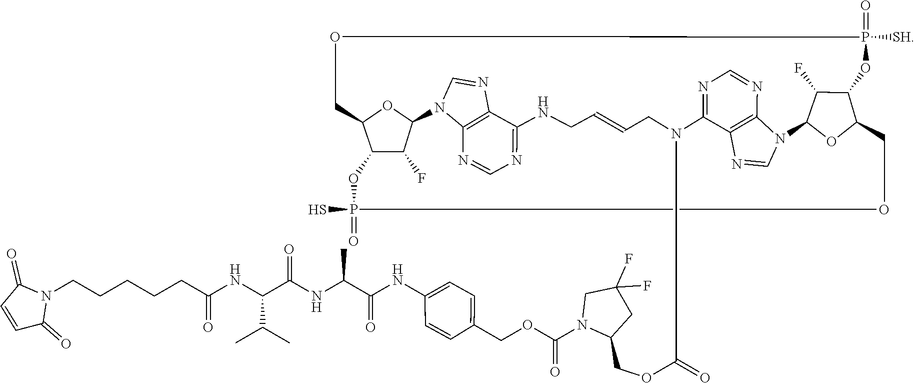 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In various embodiments, the present disclosure provides a pharmaceutical formulation comprising an anti-PSMA ADC, antibody, antigen-binding fragment thereof, or linker-payload conjugate as disclosed herein and a pharmaceutically acceptable carrier.

In various embodiments, the present disclosure provides a composition comprising multiple copies of an antibody-drug conjugate of Formula (I):

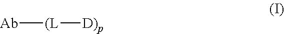

wherein
Ab is an anti-PSMA antibody or antigen-binding fragment as disclosed herein;
L-D is a linker-payload conjugate as disclosed herein; and
p is the average number of L-D moieties per Ab, wherein the average p of the antibody-drug conjugates in the composition is from about 2 to about 8.

In some embodiments, the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP16:

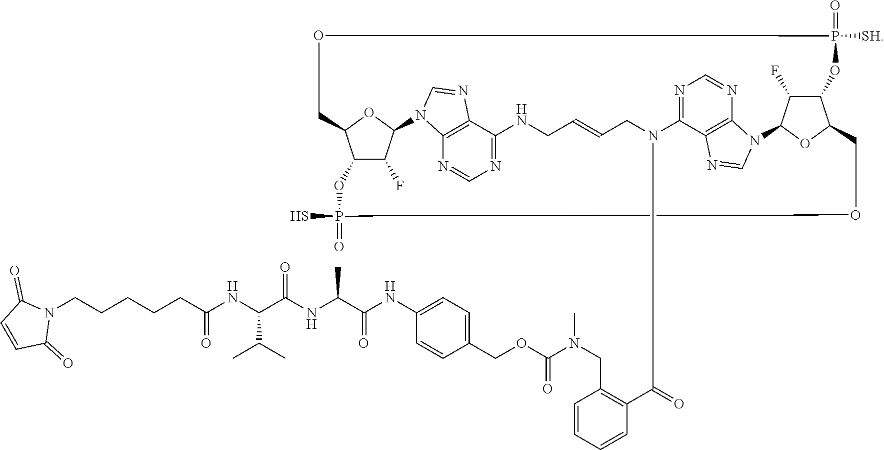 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP20:

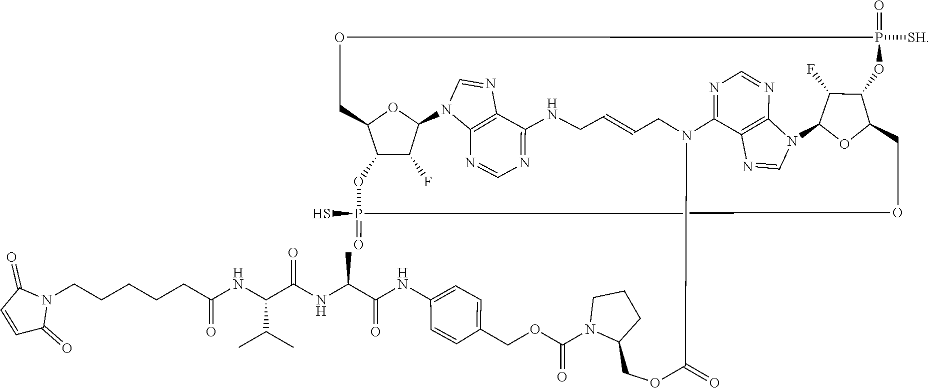 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP26:

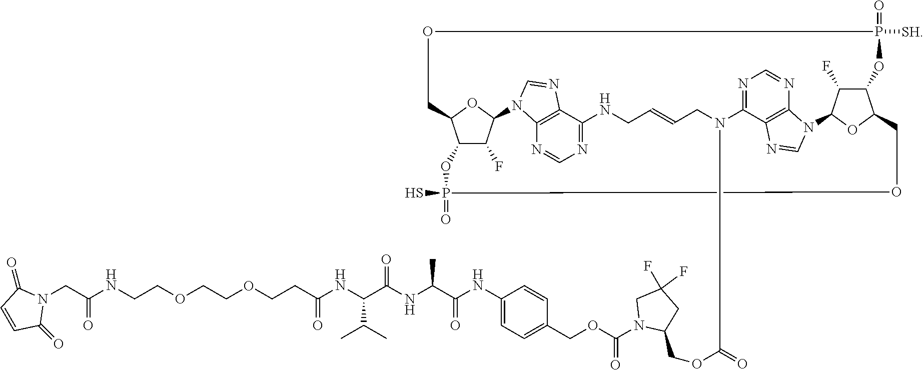 [SMILES: CC(C)[C@H](NC(=O)CCOCCOCCNC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP28:

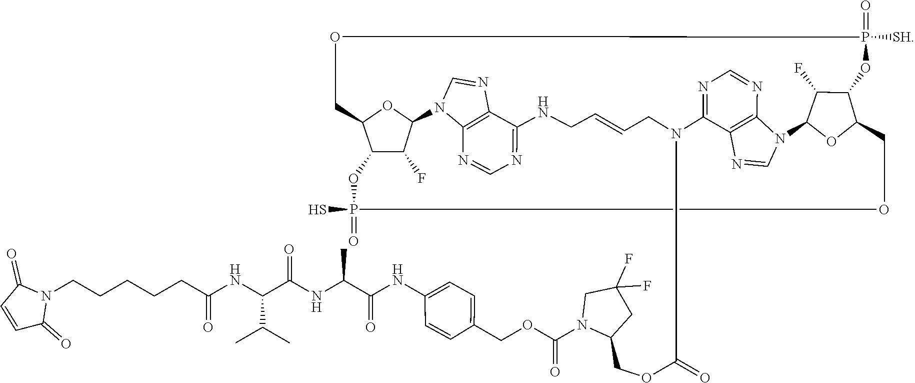 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, the antibody or antigen-binding fragment comprises a heavy chain variable region comprising an amino acid sequence of SEQ ID NO: 14, and a light chain variable region comprising an amino acid sequence of SEQ ID NO: 19.

In various embodiments, the present disclosure provides methods of treating a patient having or at risk of having a cancer, comprising administering to the patient a therapeutically effective amount of an anti-PSMA ADC, antibody, antigen-binding fragment thereof, or linker-payload conjugate as disclosed herein. In various embodiments, the present disclosure provides methods of reducing or inhibiting growth of a cancer, comprising administering a therapeutically effective amount of an anti-PSMA ADC, antibody, antigen-binding fragment thereof, or linker-payload conjugate as disclosed herein. In some embodiments, the cancer expresses PSMA. In some embodiments, the cancer is prostate cancer.

In various embodiments, the present disclosure provides use of an anti-PSMA ADC, antibody, antigen-binding fragment thereof, or linker-payload conjugate as disclosed herein in the treatment of a cancer. In some embodiments, the cancer expresses PSMA. In some embodiments, the cancer is prostate cancer.

In various embodiments, the present disclosure provides methods of producing an anti-PSMA ADC, comprising reacting an antibody or antigen-binding fragment thereof as disclosed herein with a linker-payload conjugate as disclosed herein. In various embodiments, the present disclosure provides methods of producing an antibody-drug conjugate, wherein the method comprises conjugating an antibody or antigen-binding fragment as disclosed herein with a linker-payload conjugate as disclosed herein under conditions suitable for attachment.

In various embodiments, the present disclosure provides methods of producing an L-D conjugate (V):

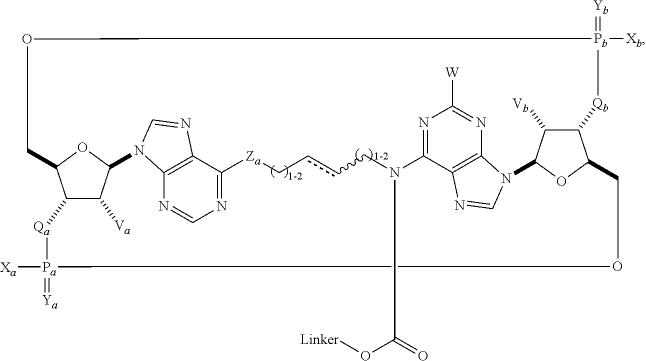

the method comprising reacting a compound of Formula (III) as disclosed herein:

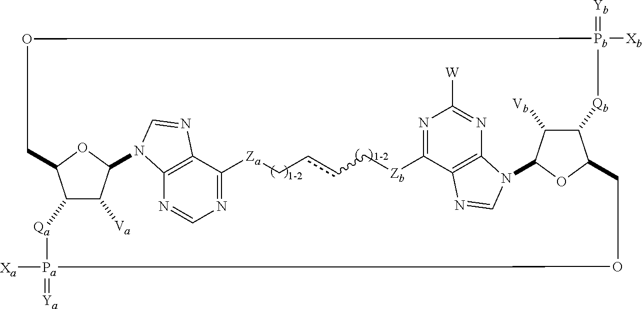

or a salt thereof with an activated linker comprising a suitable linker having the following structure:

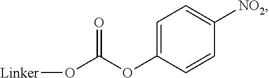

to produce the L-D conjugate (V),
wherein Z_{b} is NH.

In some embodiments, P_{b} has (S)-configuration, and the activated linker reacts with Z_{b} preferentially. In some embodiments, the compound of Formula (III) is Compound 1.

In various embodiments, the present disclosure provides methods of producing an L-D conjugate (VI):

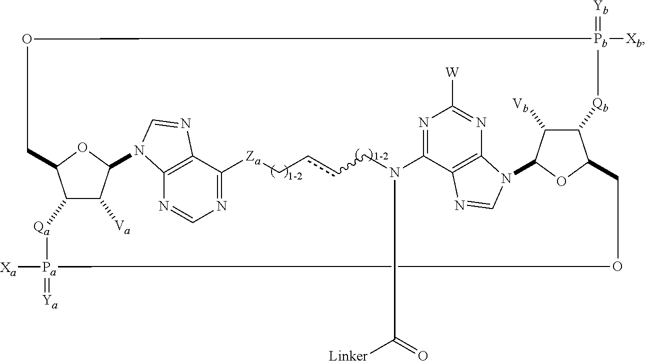

the method comprising reacting a compound of Formula (III) as disclosed herein:

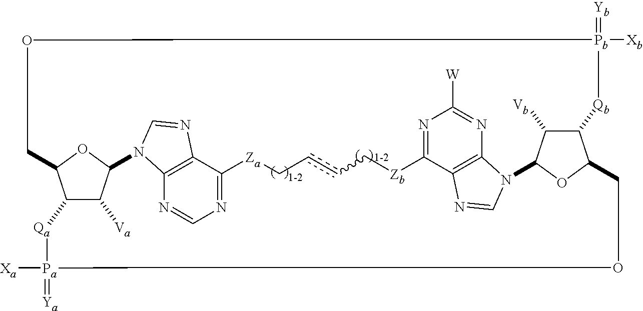

or a salt thereof with an activated linker comprising a suitable linker having the following structure:

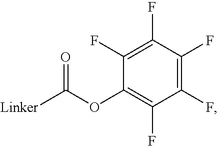

to produce the L-D conjugate (VI),
wherein Z_{b} is NH.

In some embodiments, P_{b} has (S)-configuration, and the activated linker reacts with Z_{b} preferentially. In some embodiments, the compound of Formula (III) is Compound 1.

In various embodiments, the present disclosure provides compositions comprising a linker-payload conjugate as disclosed herein. In some embodiments, the present disclosure provides a composition comprising a linker-payload conjugate produced according to the methods disclosed herein.

### BRIEF DESCRIPTION OF THE DRAWINGS

FIG. 1 shows alignment of J591 VH and VL with human germline sequences. Underlined residues are human-specific residues, lowercase residues are mouse-specific residues.

FIG. 2 shows an in silico model of J591 Fv that was generated using BioLuminate software. CDR residues are shown as space-fill, framework residues differing between mouse and HCzu1-Lczu1 that are adjacent to the CDRs are shown as ball-stick. Residue numbering is according to Kabat.

FIG. 3 shows similar PSMA binding for humanized heavy chain (HC) variants 1-10 paired with Lczu1. Hczu1-10 were paired with LCzu1 and analyzed for binding to PSMA by ELISA.

FIG. 4 shows super-humanization of J591. The resulting PSMA antibodies have strong binding affinity. Humanized J591 variants were analyzed for binding to PSMA by ELISA.

FIG. 5A shows thermal stability of deJ591. FIG. 5B shows thermal stability of humanized J591 variants HC1-LC1, HC2-LC1, HC3-LC1, HC14-LC1, and HC14-LC5 compared to deJ591. FIG. 5C shows thermal stability of HC14-LC5 (H14L5) IgG1 antibodies modified to include site-specific conjugation residues compared to deJ591 and J591.

FIG. 6A shows immunogenicity prediction scores for 9mer peptide sequences on the heavy chain variable domain of J591. FIG. 6B shows immunogenicity prediction scores for 9mer peptide sequences on the heavy chain variable domain of deJ591. FIG. 6C shows immunogenicity prediction scores for 9mer peptide sequences on the heavy chain variable domain of zuJ591-H14.

FIG. 7A shows immunogenicity prediction scores for 9mer peptide sequences on the light chain variable domain of J591. FIG. 7B shows immunogenicity prediction scores for 9mer peptide sequences on the light chain variable domain of deJ591. FIG. 7C shows immunogenicity prediction scores for 9mer peptide sequences on the light chain variable domain of zuJ591-L5.

FIG. 8 shows anti-PSMA-specific binding to PSMA-expressing LNCaP cells.

FIG. 9 shows PSMA-dependent ADCP activity as assessed by flow cytometry. The percentage of macrophages that ingested at least one target cell is shown.

FIG. 10 shows target cell-dependence of anti-PSMA ADC internalization as measured by flow cytometry.

FIG. 11 shows ADCP-dependent IFNβ production by anti-PSMA ADC treatment.

FIG. 12 shows ADCP-dependent myeloid cell activation by anti-PSMA ADC treatment.

FIG. 13 shows anti-tumor activity of anti-PSMA ADC in vivo in a PSMA-positive LNCaP xenograft model.

FIG. 14A shows a heatmap of Type 1 Interferon gene expression as assessed by RNA-seq in LNCaP xenograft model treated with anti-PSMA antibody (PSMA Control), anti-PSMA-LP3 ADC, or negative control (anti-SEB-LP3). FIG. 14B shows cytokines specific to the STING pathway were modulated by anti-PSMA-LP3 ADC treatment. FIG. 14C shows macrophage polarization shift from M2 to M1 in the tumor microenvironment when treated with anti-PSMA-LP3 ADC.

FIG. 15A shows tumor volume (left) and percent body weight change (right) in human prostate cancer 22RV1 xenograft mice (castrated) upon treatment with anti-PSMA antibody or anti-PSMA ADC. FIG. 15B shows tumor volume (left) and percent body weight change (right) in human prostate cancer 22RV1 xenograft mice (uncastrated) upon treatment with anti-PSMA antibody or anti-PSMA ADC. Sp=S thiophosphate linker attachment point on Compound 1; Rp=R thiophosphate linker attachment point on Compound 1.

FIG. 16A shows a model of in vivo efficacy of anti-PSMA ADCs in a 22RV1 xenograft model.

FIG. 16B shows concentration of mouse TNFα (left) or IFNβ (right) in plasma at 6 h post-injection with anti-PSMA ADC.

FIG. 17 shows percent change in DAR of anti-PSMA ADCs.

FIGS. 18A, 18B, and 18C show DAR change over time for S-attached linkers.

FIGS. 19A, 19B, and 19C show free Compound 1 over time for S-attached linkers.

FIGS. 20A, 20B, and 20C show percent monomer over time for S-attached linkers.

FIGS. 21A, 21B, 21C, 21D, 21E, 21F, and 21G show DAR change over time for N-attached linkers.

FIGS. 22A, 22B, 22C, 22D, 22E, 22F, and 22G show free Compound 1 over time for N-attached linkers.

FIGS. 23A, 23B, 23C, 23D, 23E, 23F, and 23G show percent monomer over time for N-attached linkers.

FIG. 24A shows percentage of Compound 1 release from anti-PSMA ADCs in mouse plasma over time. FIG. 24B shows average DAR of anti-PSMA ADCs in mouse plasma over time. FIG. 24C shows percent change from starting DAR of anti-PSMA ADCs in mouse plasma. FIG. 24D shows free Compound 1 in mouse plasma over time.

FIG. 25 shows mouse plasma stability of S-linked anti-PSMA Compound 1 ADCs.

FIG. 26 shows structures of Compound 1 and monophosphate forms of Compound 1.

FIG. 27A shows average DAR of anti-PSMA ADC LP3 (random DAR4 and RESPECT-L DAR4).

FIG. 27B shows metabolism of anti-PSMA ADC LP3 random DAR4. FIG. 27C shows metabolism of anti-PSMA ADC LP3 RESPECT-L DAR4.

FIG. 28A shows stability of N-linked anti-PSMA ADCs in mouse plasma over 10 days of treatment. FIG. 28B shows stability of N-linked anti-PSMA ADCs in mouse plasma over at day 7.

FIG. 29 shows DAR of N-linked anti-PSMA Compound 1 ADCs in mouse plasma at Day 7/10.

FIG. 30 shows hIFN-ρ production in C4-2/THP1 co-culture upon treatment with anti-PSMA ADCs.

FIG. 31 shows mean tumor volume and percent body weight loss in xenograft tumors treated with anti-PSMA ADCs.

FIG. 32 shows anti-tumor activity of Anti-PSMA-LP ADCs in the 22Rv1 xenograft model (Cohort 1). Average tumor growth and average body weight change are shown.

FIG. 33 shows serum cytokine analysis for PSMA-LP ADCs in the 22Rv1 xenograft model (Cohort 1). n=3; each dot represents an individual value. Data are represented as mean±SEM.

FIG. 34 shows anti-tumor activity of anti-PSMA-LP ADCs in the 22Rv1 xenograft model (Cohort 2). Average tumor growth and average body weight change are shown.

FIG. 35 shows serum cytokine analysis for PSMA-LP ADCs in the 22Rv1 xenograft model (Cohort 2). n=3; each dot represents an individual value. Data are represented as mean±SEM.

FIG. 36 shows anti-tumor activity of anti-PSMA-LP ADCs in the 22Rv1 xenograft model (Cohort 3). Average tumor growth and average body weight change are shown.

FIG. 37 shows serum cytokine analysis for PSMA-LP ADCs in the 22Rv1 xenograft model (Cohort 3). n=3; each dot represents an individual value. Data are represented as mean±SEM.

FIG. 38 shows anti-tumor activity of anti-PSMA-LP ADCs in the C4-2 xenograft model. Average tumor growth and average body weight change are shown.

FIG. 39 shows serum cytokine analysis for PSMA-LP ADCs in the 22Rv1 xenograft model (Cohort 3). n=3; each dot represents an individual value. Data are represented as mean±SEM.

FIG. 40 shows pharmacokinetics of anti-PSMA LP3 ADC (random DAR4) in normal mice at 1 mpk IV dose.

FIG. 41 shows pharmacokinetics of anti-PSMA LP3 ADC (RESPECT-L DAR2) in C4-2 tumor-bearing mice at 3 and 9 mg/kg IV dose.

FIG. 42 shows levels of Compound 1 in plasma from C4-2 tumor-bearing mice dosed with anti-PSMA LP3 ADC (RESPECT-L DAR2) or anti-PSMA LP1 ADC (RESPECT-L DAR4).

FIG. 43 shows intra-tumoral levels of Compound 1 in C4-2 tumor-bearing mice dosed with anti-PSMA LP3 ADC (RESPECT-L DAR2) or anti-PSMA LP1 ADC (RESPECT-L DAR4).

FIG. 44 shows tumor PK parameters from C4-2 tumor-bearing mice dosed with anti-PSMA LP3 ADC (RESPECT-L DAR2) and anti-PSMA LP1 ADC (RESPECT-L DAR4).

FIG. 45 shows a scheme of a two-step payload release assay. LP2 is shown as an example.

FIG. 46 shows in vitro IFN-β release after treatment with anti-PSMA S-attachment ADCs. Panels labeled A (left column) show IFN-β release from THP-1 monoculture and panels labeled B (right column) show IFN-β release from C4-2 and THP-1 co-culture.

FIG. 47 shows in vitro IFN-β release after treatment with anti-PSMA N-attachment ADCs. Panels labeled A (left column) show IFN-β release from THP-1 monoculture and panels labeled B (right column) show IFN-β release from C4-2 and THP-1 co-culture.

### DETAILED DESCRIPTION

The disclosed compositions and methods may be understood more readily by reference to the following detailed description taken in connection with the accompanying figures, which form a part of this disclosure.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise.

All references cited herein are incorporated by reference for any purpose. Where a reference and the specification conflict, the specification will control.

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise.

The terms “about” or “approximately” in the context of numerical values and ranges refers to values or ranges that approximate or are close to the recited values or ranges such that the embodiment may perform as intended, such as having a desired amount of nucleic acids or polypeptides in a reaction mixture, as is apparent to the skilled person from the teachings contained herein. In some embodiments, “about” means plus or minus 10% of a numerical amount.

The term “agent” is used herein to refer to a chemical compound, a mixture of chemical compounds, a biological macromolecule, or an extract made from biological materials. The term “therapeutic agent,” “drug,” or “drug moiety” refers to an agent that is capable of modulating a biological process and/or has biological activity.

As used herein, the term “aliphatic” or “aliphatic group” means a straight-chain (i.e., unbranched) or branched, substituted, or unsubstituted hydrocarbon chain that is completely saturated or that contains one or more units of unsaturation. In some embodiments, aliphatic groups contain 1-8 aliphatic carbon atoms. In some embodiments, aliphatic groups contain 1-6 aliphatic carbon atoms. In some embodiments, aliphatic groups contain 1-4 aliphatic carbon atoms. In some embodiments, aliphatic groups contain 4 aliphatic carbon atoms.

As used herein, the term “ambient conditions” means room temperature, open air condition and uncontrolled humidity condition. The terms “room temperature” and “ambient temperature” mean 15° C. to 30° C.

The terms “antibody-drug conjugate,” “antibody conjugate,” “conjugate,” “immunoconjugate,” and “ADC” are used interchangeably, and refer to a compound or derivative thereof that is linked to an antibody (e.g., an anti-PSMA antibody) and may be defined by the generic formula: Ab-(L-D)p (Formula I), wherein Ab=an antibody moiety (i.e., antibody or antigen-binding fragment), L=a linker moiety, D=a drug moiety, and p=the number of drug moieties per antibody moiety. In some embodiments, the linker L can include a cleavable moiety between the antibody or antigen-binding fragment and the therapeutic compound. In some embodiments, the linker L can include a cleavable moiety that can be attached to either or both the antibody or antigen-binding fragment and to the therapeutic compound, e.g., by spacer unit(s). Exemplary cleavable linkers are described and exemplified herein.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. The heavy chain (HC) of an antibody is composed of a heavy chain variable domain (VH) and a heavy chain constant region (CH). The light chain (LC) is composed of a light chain variable domain (VL) and a light chain constant domain (CL). As used herein, the terms “domain” and “region” may be used interchangeably (e.g., the term “variable domain” may be used interchangeably with the term “variable region” and understood to refer to the same part of the antibody). For the purposes of this application, the mature heavy chain and light chain variable domains each comprise three complementarity determining regions (CDR1, CDR2, and CDR3; also referred to as “hypervariable regions”) within four framework regions (FR1, FR2, FR3, and FR4) arranged from N terminus to C terminus: FR1, CDR1, FR2, CDR2, FR3, CDR3, and FR4. CDRs may be identified according to the Kabat and/or IMGT numbering systems (Kabat, Sequences of Proteins of Immunological Interest (National Institutes of Health, Bethesda, Md. (1987 and 1991); International ImMunoGeneTics Information System (IMGT®)). An “antibody” can be naturally occurring or man-made, such as monoclonal antibodies produced by conventional hybridoma technology. The term “antibody” includes full-length monoclonal antibodies and full-length polyclonal antibodies, as well as antibody fragments such as Fab, Fab′, F(ab′)2, Fv, and single chain antibodies. An antibody can be any one of the five major classes of immunoglobulins: IgA, IgD, IgE, IgG, and IgM, or subclasses thereof (e.g., isotypes IgG1, IgG2, IgG3, IgG4). An antibody of any of the aforementioned classes or subclasses can also comprise one of two functionally similar classes of light chains: Igκ (also referred to herein as “Ig kappa” or “kappa”) and Igλ (also referred to herein as “Ig lambda” or “lambda”). The term “antibody” encompasses human antibodies, chimeric antibodies, humanized antibodies, and any modified immunoglobulin molecule containing an antigen recognition site, so long as it demonstrates the desired biological activity.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with desired specificity, affinity, and activity characteristics, while the constant regions are homologous to antibodies derived from another species (e.g., human) to minimize an immune response in the latter species.

The term “human antibody,” as used herein, refers to an antibody produced by a human or an antibody having an amino acid sequence of an antibody produced by a human.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable regions correspond to those of a non-human immunoglobulin, and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity. The humanized antibody can also be further modified by the substitution of residues in the Fc domain to reduce its binding to various cell receptors, such as an Fcγ receptor (FcγR), and other immune molecules.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods. See, e.g., U.S. Pat. No. 4,816,567. Monoclonal antibodies may also be isolated from phage antibody libraries using the techniques described in, e.g., Clackson et al. (1991) Nature 352:624-8, and Marks et al. (1991) J. Mol. Biol. 222:581-97.

The monoclonal antibodies described herein specifically include “chimeric” antibodies, in which a portion of the heavy and/or light chain is identical with or homologous to corresponding sequences in an antibody derived from a particular species or belonging to a particular antibody class or subclass, while the remainder of the chain(s) is identical with or homologous to corresponding sequences in an antibody derived from another species or belonging to another antibody class or subclass, as well as fragments of such antibodies, so long as they specifically bind the target antigen and/or exhibit the desired biological activity.

The term “antigen-binding fragment” or “antigen-binding portion” of an antibody, as used herein, refers to one or more fragments of an antibody that retain the ability to specifically bind to an antigen (e.g., PSMA). Antigen-binding fragments preferably also retain the ability to internalize into an antigen-expressing cell. In some embodiments, antigen-binding fragments also retain immune effector activity. It has been shown that fragments of a full-length antibody can perform the antigen-binding function of a full-length antibody. Examples of binding fragments encompassed within the term “antigen-binding fragment” or “antigen-binding portion” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment, which comprises a single variable domain, e.g., a VH domain (see, e.g., Ward et al. (1989) Nature 341:544-6; and Winter et al., WO 90/05144); and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). See, e.g., Bird et al. (1988) Science 242:423-6; and Huston et al. (1988) Proc. Natl. Acad. Sci. USA 85:5879-83. Such single chain antibodies are also intended to be encompassed within the term “antigen-binding fragment” or “antigen-binding portion” of an antibody, and are known in the art as an exemplary type of binding fragment that can internalize into cells upon binding. See, e.g., Zhu et al. (2010) 9:2131-41; He et al. (2010) J. Nucl. Med. 51:427-32; and Fitting et al. (2015) MAbs 7:390-402. In certain embodiments, scFv molecules may be incorporated into a fusion protein. Other forms of single chain antibodies, e.g., diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites. See, e.g., Holliger et al. (1993) Proc. Natl. Acad. Sci. USA 90:6444-8; and Poljak et al. (1994) Structure 2:1121-3). Antigen-binding fragments are obtained using conventional techniques known to those of skill in the art, and the binding fragments are screened for utility (e.g., binding affinity, internalization) in the same manner as are intact antibodies. Antigen-binding fragments may be prepared, e.g., by cleavage of the intact protein, e.g., by protease or chemical cleavage.

The term “anti-PSMA antibody” or “antibody that specifically binds PSMA” refers to any form of antibody or fragment thereof that specifically binds PSMA and encompasses monoclonal antibodies (including full length monoclonal antibodies), polyclonal antibodies, and biologically functional antibody fragments so long as they specifically bind PSMA. Preferably the anti-PSMA antibody used in the ADCs disclosed herein is an internalizing antibody or internalizing antibody fragment. As used herein, the terms “specific,” “specifically binds,” and “binds specifically” refer to the selective binding of the antibody to the target antigen or epitope over alternative antigens or epitopes. Antibodies can be tested for specificity of binding by comparing binding to an appropriate antigen to binding to an irrelevant antigen or antigen mixture under a given set of conditions. If the antibody binds to the appropriate antigen with at least 2-fold, or preferably at least 50-fold, at least 100-fold, or at least 1000-fold, greater affinity than to an irrelevant antigen or antigen mixture, then it is considered to be specific, e.g., as measured by surface plasmon resonance, e.g., BIAcore® analysis. In one embodiment, a specific antibody is one that binds the PSMA antigen but does not bind (or exhibits minimal binding) to other antigens.

The term “aryl” refers to a group or substituent derived from an aromatic ring and encompasses monocyclic aromatic rings and bicyclic, tricyclic, and fused ring systems having a total of six to fourteen ring members, wherein at least one ring in the system is aromatic. An aryl group may be optionally substituted with one or more substituents.

The term “heteroaryl” refers to a cyclic group comprising at least one ring atom that is a heteroatom, such as O, N, or S. Heteroaryl groups encompass monocyclic, bicyclic, and tricyclic ring systems having a total of five to fourteen ring members, wherein at least one ring in the system is aromatic, at least one ring in the system contains one or more heteroatoms, and wherein each ring in the system contains three to seven ring members.

The term “at least one” refers to one or more.

The term “bridge” refers to a grouping of atoms in a macrocycle-bridged STING agonist compound of the disclosure that extends from a first nucleic acid base in the macrocycle-bridged STING agonist compound to a second nucleic acid base in the macrocycle-bridged STING agonist compound.

The term “cancer” refers to the physiological condition in mammals in which a population of cells is characterized by unregulated cell growth. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, bile duct cancer (e.g., cholangiocarcinoma), esophageal cancer, nasopharyngeal cancer, cancer of the peritoneum, hepatocellular cancer (e.g., hepatocellular carcinoma), gastrointestinal cancer, pancreatic cancer, glioblastoma, cervical cancer, ovarian cancer, liver cancer, bladder cancer, hepatoma, breast cancer, osteosarcoma, skin cancer (e.g., melanoma), colon cancer, colorectal cancer, endometrial or uterine cancer, ovarian cancer, salivary gland carcinoma, kidney cancer, liver cancer, prostate cancer (e.g., advanced prostate cancer, metastatic castration-resistant prostate cancer), vulval cancer, thyroid cancer, hepatic carcinoma, bone cancer and various types of head and neck cancers.

The terms “cancer cell” and “tumor cell” refer to individual cells or the total population of cells derived from a tumor, including both non-tumorigenic cells and cancer stem cells. As used herein, the term “tumor cell” will be modified by the term “non-tumorigenic” when referring solely to those tumor cells lacking the capacity to renew and differentiate to distinguish those tumor cells from cancer stem cells.

The terms “tumor” and “neoplasm” refer to any mass of tissue that results from excessive cell growth or proliferation, either benign or malignant, including precancerous lesions.

The term “chemotherapeutic agent” or “anti-cancer agent” is used herein to refer to a chemical compound that is effective in treating cancer regardless of mechanism of action. Inhibition of metastasis or angiogenesis is frequently a property of a chemotherapeutic agent. Stimulation of an antitumor immune response may also be a property of a chemotherapeutic agent. Non-limiting examples of chemotherapeutic agents include stimulatory agents, e.g., STING agonists. In addition, chemotherapeutic agents include antibodies, biological molecules, and small molecules. A chemotherapeutic agent may be a cytotoxic or cytostatic agent.

The term “cytotoxic agent” refers to a substance that causes cell death either by interfering with a cell's expression activity and/or functioning or by stimulating a response that causes cell death, e.g., an immune response. Examples of cytotoxic agents include, but are not limited to, STING agonists such as Compound 1.

An “effective amount” of an ADC as disclosed herein is an amount sufficient to perform a specifically stated purpose, for example to produce a therapeutic effect after administration, such as a reduction in tumor growth rate or tumor volume, a reduction in a symptom of cancer, or some other indicia of treatment efficacy. An effective amount can be determined in a routine manner in relation to the stated purpose. The term “therapeutically effective amount” refers to an amount of an ADC effective to treat a disease or disorder in a subject. In the case of cancer, a therapeutically effective amount of ADC can reduce the number of cancer cells, reduce tumor size, inhibit (e.g., slow or stop) tumor metastasis, inhibit (e.g., slow or stop) tumor growth, and/or relieve one or more symptoms. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “epitope” refers to the portion of an antigen capable of being recognized and specifically bound by an antibody. When the antigen is a polypeptide, epitopes can be formed from contiguous amino acids or noncontiguous amino acids juxtaposed by tertiary folding of the polypeptide. The epitope bound by an antibody may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry. See, e.g., Gershoni et al. (2007) 21:145-56; and Hager-Braun and Tomer (2005) Expert Rev. Proteomics 2:745-56).

The term “Compound 1,” as used herein, refers to the structure of Compound 1 shown below, or a salt thereof:

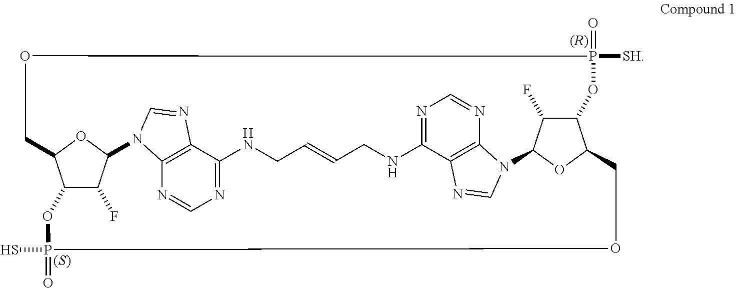

Compound 1 is a macrocycle-bridged STING agonist (MBSA) with a locked bioactive U-shaped conformation of cyclic dinucleotides comprising a transannular macrocyclic bridge between the nucleic acid bases. As used herein, “Compound 1” may include salts of Compound 1, e.g., diammonium salt and/or sodium salt of Compound 1. The term “Compound 1 moiety,” “E7766,” “E7766 agonist moiety,” or “E7766 moiety” refers to the component of an ADC that has the structure of Compound 1, and is attached to the linker of the ADC, e.g., via its N-34 nitrogen, N-39 nitrogen, S-2 sulfur, or S-14 sulfur of the Compound 1 moiety. Compositions and methods of inhibiting tumor growth in patients comprising administering Compound 1 are disclosed in WO 2018/152450, which is incorporated herein by reference in its entirety for all Compound 1 structures and methods of synthesizing those structures.

Atoms in Compound 1, as referenced herein, may be numbered as shown below:

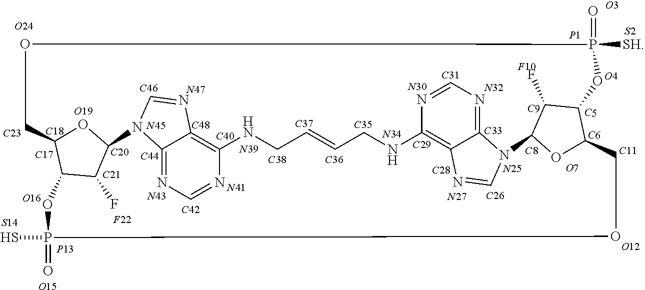

The term “Compound 2,” as used herein, refers to the structure of Compound 2 shown below, or a salt thereof:

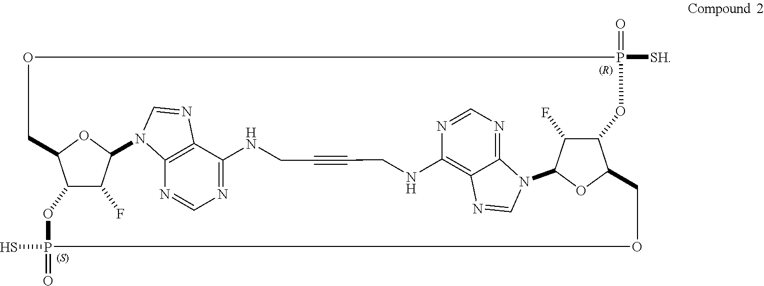

Atoms in Compound 2, as referenced herein, may be numbered as shown below:

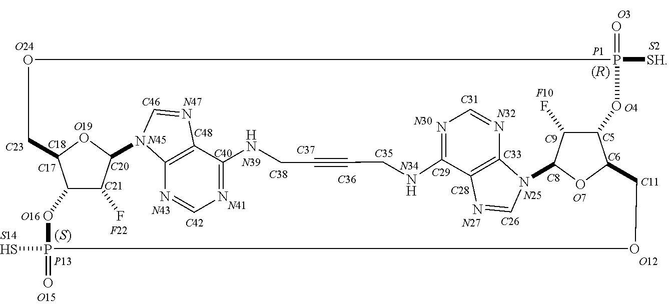

In various embodiments of the disclosure, “N-34 nitrogen,” “N-39 nitrogen,” “S-2 sulfur,” or “S-14 sulfur” may be used to refer to the nitrogen or sulfur atoms in other STING agonists that correspond to the numbered nitrogen or sulfur atoms in Compound 1 or Compound 2, regardless of whether the atoms would be numbered otherwise according to the naming convention. In some instances, for compounds of Formula (III), Formula (IV), or Table 14, e.g., Compound 1 or Compound 2, an L-D conjugate with attachment at the N-34 nitrogen may be referred to as “RN” or “RN,” and an L-D conjugate with attachment at the N-39 nitrogen may be referred to as “SN” or “SN.”

“Fcγ receptor,” “Fc-gamma receptor,” or “FcγR” refers to a cell surface protein generally found on immune cells of various types, e.g., neutrophils. The binding of an Fc region of an antibody to a Fcγ receptor may induce different effector functions, for example antibody-dependent cellular cytotoxicity (ADCC) or antibody-dependent cellular phagocytosis (ADCP).

As used herein, the term “halogen” or “halo” means F, Cl, Br, or I.

The term “homolog” refers to a molecule which exhibits homology to another molecule, by, for example, having sequences of chemical residues that are the same or similar at corresponding positions.

The terms “IgG1 Fc,” “IgG1 Fc domain” or “IgG1 Fc-containing antibody” as used herein refer to an antibody having at least an IgG1 CH2 and CH3 domain, as identified by SEQ ID NO: 70 and SEQ ID NO: 71, respectively.

“Wild type IgG1 Fc domain” refers to a human IgG1 Fc domain that comprises the amino acid sequence of SEQ ID NO: 69 or a fragment thereof.

The term “inhibit,” or “inhibition of,” as used herein, means to reduce by a measurable amount, and can include but does not require complete prevention or inhibition.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-PSMA antibody is one that is capable of being taken into the cell after binding to PSMA on the cell membrane.

The term “KD” refers to the equilibrium dissociation constant of a particular antibody-antigen interaction. KD is calculated by kₐ/k_{d}. The rate can be determined using standard assays, such as a BIAcore® or ELISA assay.

The term “kₒₙ” or “kₐ” refers to the on-rate constant for association of an antibody to the antigen to form the antibody/antigen complex. The rate can be determined using standard assays, such as a BIAcore® or ELISA assay.

The term “k_{off}” or “k_{d}” refers to the off-rate constant for dissociation of an antibody from the antibody/antigen complex. The rate can be determined using standard assays, such as a BIAcore® or ELISA assay.

A “linker” or “linker moiety” is any chemical moiety that is capable of covalently joining a compound, usually a drug moiety such as a chemotherapeutic agent, to another moiety such as an antibody moiety. Linkers can be susceptible to or substantially resistant to acid-induced cleavage, peptidase-induced cleavage, light-based cleavage, esterase-induced cleavage, and/or disulfide bond cleavage, at conditions under which the compound or the antibody remains active. A “cleavable linker” is any linker that comprises a cleavable moiety and can thus be susceptible to cleavage. A cleavable moiety can be a cleavable peptide moiety. The term “cleavable peptide moiety” refers to any chemical bond linking amino acids (natural or synthetic amino acid derivatives) that can be cleaved by an agent that is present in the intracellular environment.

The use of “or” will mean “and/or” unless the specific context of its use dictates otherwise.

The term “p” or “antibody:drug ratio” or “drug-to-antibody ratio” or “DAR” refers to the number of drug moieties per antibody moiety, i.e., drug loading, or the number of L-D moieties per antibody or antigen-binding fragment (Ab) in ADCs of Formula I. In compositions comprising multiple copies of ADCs of Formula I, “p” refers to the average number of L-D moieties per antibody or antigen-binding fragment, also referred to as average drug loading.

A “pharmaceutical composition” refers to a preparation which is in such form as to permit administration and subsequently provide the intended biological activity of the active ingredient(s) and/or to achieve a therapeutic effect, and which contains no additional components which are unacceptably toxic to a subject to which the formulation would be administered. The pharmaceutical composition may be sterile.

A “pharmaceutical excipient” comprises a material such as an adjuvant, a carrier, pH-adjusting and buffering agents, tonicity adjusting agents, wetting agents, preservatives, and the like.

“Pharmaceutically acceptable” means approved or approvable by a regulatory agency of the Federal or a state government, or listed in the U.S. Pharmacopeia or other generally recognized pharmacopeia, for use in animals, and more particularly in humans.

As used herein, the term “pi bond” means a covalent bond formed by the p orbitals of adjacent atoms. Pi bonds exist where there is a multiple bond, i.e., a double or triple bond, between two atoms. For example, a carbon-carbon double bond consists of one pi bond, and a carbon-carbon triple bond consists of two pi bonds.

The term “prostate-specific membrane antigen” or “PSMA,” as used herein, refers to any native form of human PSMA. The term encompasses full-length PSMA (e.g., NCBI Reference Sequence: NP_004467.1; SEQ ID NO: 67), as well as any form of human PSMA that results from cellular processing. The term may also encompass naturally occurring variants of PSMA, including but not limited to splice variants, allelic variants, and isoforms. An antibody that binds PSMA may not bind all variants, as will be readily apparent to one of skill in the art. PSMA can be isolated from a human or may be produced recombinantly or by synthetic methods. The terms “PSMA” and “prostate-specific membrane antigen” are interchangeable with “glutamate carboxypeptidase II (GCPII),” “folate hydrolase 1,” “N-acetylated-alpha-linked acidic dipeptidase I (NAALADase I)” and any other name for proteins encoded by FOLH1 known in the art.

The term “protecting group,” as used herein, refers to any chemical group introduced into a molecule by chemical modification of a functional group to obtain chemoselectivity in a subsequent chemical reaction.

Methods of adding (a process generally referred to as “protecting”) and removing (process generally referred to as “deprotecting”) protecting groups are well-known in the art and available, for example, in P. J. Kocienski, Protecting Groups, 3rd edition (Thieme, 2005), and in Greene and Wuts, Protective Groups in Organic Synthesis, 4th edition (John Wiley & Sons, New York, 2007), both of which are hereby incorporated by reference in their entirety.

Non-limiting examples of useful protecting groups for amines that may be used in this disclosure include monovalent protecting groups, for example, t-butyloxycarbonyl (Boc), benzyl (Bn), 9-fluorenylmethyloxycarbonyl (Fmoc), benzyloxycarbonyl (Cbz), formyl, acetyl (Ac), trifluoroacetyl (TFA), and p-toluenesulfonyl (Ts); and divalent protecting groups, for example, benzylidene, N-phthalimide, N-dithiasuccinimide, N-2,3-diphenylmaleimide, N-2,3-dimethylmaleimide, and N-2,5-dimethylpyrrole.

Non-limiting examples of useful protecting groups for alcohols that may be used in this disclosure include, for example, acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), 4-nitrophenyl carbonate, trimethylsilyl (TMS), triethylsilyl (TES), triisopropylsilyl (TIPS), t-butyldimethylsilyl (TBS), and t-butyldiphenylsilyl (TBDPS).

Non-limiting examples of useful protecting groups for carboxylic acids that may be used in this disclosure include, for example, methyl or ethyl esters, substituted alkyl esters such as 9-fluorenylmethyl, methoxymethyl (MOM), tetrahydropyranyl (THP), tetrahydrofuranyl, 3-methoxyethoxymethyl (MEM), 2-(trimethylsilyl)ethoxymethyl (SEM), benzyloxymethyl (BOM), acetyl (Ac), phenacyl, substituted phenacyl esters, t-butyl, allyl, phenyl (Ph), silyl esters, benzyl and substituted benzyl esters, 2,6-dialkylphenyl, and pentafluorophenyl (PFP).

Non-limiting examples of amine bases that may be used in this disclosure include, for example, 1,8-diazabicyclo[5.4.0]undec-7-ene (DBU), N-methylmorpholine (NMM), triethylamine (Et₃N; TEA), diisopropylethyl amine (i-Pr₂EtN; DIPEA), pyridine, 2,2,6,6-tetramethylpiperidine, 1,5,7-triazabicyclo[4.4.0]dec-5-ene (TBD), 7-methyl-1,5,7-triazabicyclo[4.4.0]dec-5-ene (MTBD), t-Bu-tetramethylguanidine, 1,5-diazabicyclo[4.3.0]non-5-ene (DBN), lithium bis(trimethylsilyl)amide (LiHMDS), and potassium bis(trimethylsilyl)amide (KHMDS).

Non-limiting examples of carbonate bases that may be used in this disclosure include, for example, sodium carbonate (Na₂CO₃), potassium carbonate (K₂CO₃), cesium carbonate (Cs₂CO₃), lithium carbonate (Li₂CO₃), sodium bicarbonate (NaHCO₃), and potassium bicarbonate (KHCO₃).

Non-limiting examples of phosphate bases that may be used in this disclosure include, for example, sodium phosphate tribasic (Na₃PO₄), potassium phosphate tribasic (K₃PO₄), potassium phosphate dibasic (K₂HPO₄), and potassium phosphate monobasic (KH₂PO₄).

Non-limiting examples of acids that may be used in this disclosure include, for example, acetic acid (AcOH), trifluoroacetic acid (TFA), hydrochloric acid (HCl), camphorsulfonic acid (CSA), methanesulfonic acid (MsOH), formic acid (FA), phosphoric acid (H₃PO₄), and sulfuric acid (H₂SO₄).

Non-limiting examples of peptide coupling reagents include, for example, N,N′-dicyclohexylcarbodiimide (DCC), 1-ethyl-3-[3-(dimethylamino)propyl]carbodiimide (EDCI), 4-(4,6-dimethoxy-1,3,5-triazin-2-yl)-4-methylmorpholin-4-ium chloride (DMT-MM), 1-ethoxycarbonyl-2-ethoxy-1,2-dihydroquinoline (EEDQ), 1-[bis(dimethylamino)methylene]-1H-1,2,3-triazolo[4,5-b]pyridinium 3-oxid hexafluorophosphate (HATU), 1-hydroxybenzotriazole (HOBT), and N,N,N,N′-tetramethyl-O-(N-succinimidyl)uronium tetrafluoroborate (TSTU).

For amino acid sequences, sequence identity and/or similarity may be determined using standard techniques known in the art, including, but not limited to, the local sequence identity algorithm of Smith and Waterman (1981) Adv. Appl. Math. 2:482, the sequence identity alignment algorithm of Needleman and Wunsch (1970) J. Mol. Biol. 48:443, the search for similarity method of Pearson and Lipman (1988) Proc. Nat. Acad. Sci. USA 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (1984) Nucl. Acid Res. 12:387-95, preferably using the default settings, or by inspection. Preferably, percent identity is calculated by FastDB based upon the following parameters: mismatch penalty of 1; gap penalty of 1; gap size penalty of 0.33; and joining penalty of 30. See “Current Methods in Sequence Comparison and Analysis,” Macromolecule Sequencing and Synthesis, Selected Methods and Applications, pp. 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-60; the method is similar to that described by Higgins and Sharp (1989) CABIOS 5:151-3. Useful PILEUP parameters including a default gap weight of 3.00, a default gap length weight of 0.10, and weighted end gaps.

Another example of a useful algorithm is the BLAST algorithm. See, e.g., Altschul et al. (1990) J. Mol. Biol. 215:403-10; Altschul et al. (1997) Nucleic Acids Res. 25:3389-402; and Karin et al. (1993) Proc. Natl. Acad. Sci. USA 90:5873-87. A particularly useful BLAST program is the WU-BLAST-2 program which was obtained from Altschul et al. (1996) Methods in Enzymology 266:460-80. WU-BLAST-2 uses several search parameters, most of which are set to the default values. The adjustable parameters are set with the following values: overlap span=I, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (1993) Nucl. Acids Res. 25:3389-402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, proteins disclosed herein and variants thereof (e.g., variants that retain function of the original protein), including variants of PSMA and variants of antibody variable domains (including individual variant CDRs), have amino acid homology, similarity, or identity of at least 80%, and more typically homologies or identities of at least 85%, 90%, 91%, 92%, 93%, 94%, 95%, 96%, 97%, 98%, 99%, and almost 100% or 100%.

In a similar manner, “percent (%) nucleic acid sequence identity,” with respect to the nucleic acid sequence of the antibodies and other proteins identified herein, is defined as the percentage of nucleotide residues in a candidate sequence that are identical with the nucleotide residues in the coding sequence of the antigen binding protein. A specific method uses the BLASTN module of WU-BLAST-2 set to the default parameters, with overlap span and overlap fraction set to 1 and 0.125, respectively.

The term “stable,” as used herein, refers to compounds that are not substantially altered when subjected to conditions to allow for their production, detection, and preferably their recovery, purification, and use for one or more of the purposes disclosed herein.

The terms “subject” and “patient” are used interchangeably herein to refer to any animal, such as any mammal, including but not limited to, humans, non-human primates, rodents, and the like. In some embodiments, the mammal is a mouse. In some embodiments, the mammal is a human.

The term “target-negative” or “target antigen-negative” refers to the absence of target antigen expression by a cell or tissue. The term “target-positive” or “target antigen-positive” refers to the presence of target antigen expression. For example, a cell or a cell line that does not express a target antigen may be described as target-negative, whereas a cell or cell line that expresses a target antigen may be described as target-positive.

As used herein, the term “solvent” refers to any liquid in which the product is at least partially soluble (solubility of product >1 g/L).

As used herein, the term “isomer” refers to compounds with identical molecular formula but distinct spatial arrangement of atoms or bonds. Isomers include stereoisomers, cis-trans isomers, atropisomers, and tautomers.

As used herein, the term “stereoisomer” refers to both enantiomers and diastereomers.

It will be appreciated that certain compounds of this invention may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e., one stereoisomer). As used herein, a stereogenic atom that is notated with an (R) or (S) indicates the stereochemical designation of the stereogenic atom under the Cahn-Ingold-Prelog convention. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn.

Certain compounds disclosed herein may exist as tautomers and both tautomeric forms are intended, even though only a single tautomeric structure is depicted.

The disclosure also provides processes for preparing salts of the compounds of the disclosure.

A salt of a compound of this disclosure is formed between an acid and basic group(s) of the compound, such as an amino functional group, or a base and acidic group(s) of the compound, such as a carboxyl functional group. Depending on the ratio of the basic or acidic group(s) in the compound to the valence of the acid or base, one compound may form salt with one or more molecular units of the acid/base, or multiple units of the compound may form salt with one unit of the acid/base. In some embodiments, the salt is a sodium salt. In some embodiments, the salt is a diammonium salt. In some embodiments, the salt is a dialkylammonium salt. In some embodiments, the salt is a bis(triethylammonium) salt.

The term “stimulator of interferon genes” or “STING,” as used herein, refers to any native form of human STING. The term encompasses full-length STING (e.g., NCBI Reference Sequence: NP_938023.1; SEQ ID NO: 68), as well as any form of human STING that results from cellular processing. The term also encompasses naturally occurring variants of STING, including but not limited to splice variants, allelic variants, and isoforms. STING can be isolated from a human, or may be produced recombinantly or by synthetic methods.

As used herein, “to treat” or “therapeutic,” and grammatically related terms, refer to any improvement of any consequence of disease, such as prolonged survival, less morbidity, and/or a lessening of side effects which are the byproducts of an alternative therapeutic modality. As is readily appreciated in the art, full eradication of disease is preferred but albeit not a requirement for a treatment act. “Treatment” or “treat,” as used herein, refers to the administration of a described ADC or antibody to a subject, e.g., a patient. The treatment can be to cure, heal, alleviate, relieve, alter, remedy, ameliorate, palliate, improve, or affect the disorder, the symptoms of the disorder or the predisposition toward the disorder, e.g., a cancer.

As used herein, the term “unsaturated” means that a moiety has one or more units of unsaturation.

### Anti-PSMA Antibodies and Antigen-Binding Fragments

The present disclosure provides antibodies that specifically bind to PSMA and may be used alone, e.g., formulated as therapeutic or diagnostic antibody compositions, e.g., for use in treating or detecting PSMA-expressing cancers. The antibodies may be provided packaged or prepared for therapeutic use as antibodies, antigen binding fragments thereof, or as portions of ADCs.

The antibodies disclosed herein may bind to PSMA with a dissociation constant (KD) of ≤1 mM, ≤100 nM, or ≤10 nM, or any amount in between, as measured by, e.g., BIAcore® analysis. In some embodiments, the KD is 500 pM to 1 nM, or 1 nM to 10 nM. In some embodiments, the KD is ≤10 nM, ≤5 nM, ≤1 nM, or ≤0.5 nM.

In some embodiments, the antibodies are four-chain antibodies (also referred to as an immunoglobulin), comprising two heavy chains and two light chains. In some embodiments, the antibodies are two-chain half bodies (one light chain and one heavy chain), or antigen-binding fragments of an immunoglobulin.

In some embodiments, the antibodies are internalizing antibodies or internalizing antigen-binding fragments thereof. In some embodiments, the internalizing antibodies bind to PSMA expressed on the surface of a cell and enter the cell upon or after binding. In some embodiments, the drug moiety of the ADC is released from the antibody moiety of the ADC after the ADC enters and is present in a cell expressing PSMA (i.e., after the ADC has been internalized). In some embodiments, the internalizing antibodies bind to PSMA expressed on the cell surface of a cell and the cell is subsequently phagocytosed (e.g., antibody-dependent cellular phagocytosis occurs). In some embodiments, the drug moiety of the ADC is released from the antibody moiety of the ADC after the ADC enters and is present in the phagocytic cell (e.g., macrophage, dendritic cell).

The antibodies disclosed herein that specifically bind a PSMA protein may comprise three heavy chain CDRs (HCDR1, HCDR2, and HCDR3) having amino acid sequences selected from the HC CDRs listed in Tables 1 and/or 3, infra, as defined by the Kabat numbering system, and three light chain CDRs (LCDR1, LCDR2, and LCDR3) having amino acid sequences selected from the LC CDRs listed in Tables 1 and/or 3, infra, as defined by the Kabat numbering system. In some embodiments, the antibodies comprise three heavy chain CDRs (HCDR1, HCDR2, and HCDR3) having amino acid sequences selected from the HC CDRs listed in Table 5, infra, as defined by the IMGT numbering system, and three light chain CDRs (LCDR1, LCDR2, and LCDR3) having amino acid sequences selected from the LC CDRs listed in Table 5, infra, as defined by the IMGT numbering system.

In some embodiments, an antibody disclosed herein comprises a VH domain having an amino acid sequence selected from SEQ ID NOs: 1-14 listed in Tables 2 and/or 7, infra. In some embodiments, the antibody comprises a VL domain having an amino acid sequence selected from SEQ ID NOs: 15-20 listed in Tables 2 and/or 7, infra.

In some embodiments, an antigen-binding fragment disclosed herein retains PSMA binding. In some embodiments, the antigen binding fragment retains PSMA binding by comprising three heavy chain CDRs (HCDR1, HCDR2, and HCDR3) comprising amino acid sequences selected from the HC CDRs listed in Tables 1 and/or 3, infra, as defined by the Kabat numbering system, and three light chain CDRs (LCDR1, LCDR2, and LCDR3) comprising amino acid sequences selected from the LC CDRs listed in Tables 1 and/or 3, infra, as defined by the Kabat numbering system. In some embodiments, the antigen binding fragment comprises three heavy chain CDRs (HCDR1, HCDR2, and HCDR3) comprising amino acid sequences selected from the HC CDRs listed in Table 5, infra, as defined by the IMGT numbering system, and three light chain CDRs (LCDR1, LCDR2, and LCDR3) comprising amino acid sequences selected from the LC CDRs listed in Table 5, infra, as defined by the IMGT numbering system. In some embodiments, the antigen-binding fragments disclosed herein may retain PSMA binding by comprising a VH domain comprising an amino acid sequence selected from SEQ ID NOs: 1-14 listed in Tables 2 and/or 7, infra, and a VL domain comprising an amino acid sequence selected from SEQ ID NOs: 15-20 listed in Tables 2 and/or 7, infra.

In some embodiments, the anti-PSMA antibody or antigen-binding fragment comprises three HCDRs comprising amino acid sequences of SEQ ID NO: 21 (HCDR1), SEQ ID NO: 44 (HCDR2), and SEQ ID NO: 27 (HCDR3); and three LCDRs comprising SEQ ID NO: 45 (LCDR1), SEQ ID NO: 46 (LCDR2), and SEQ ID NO: 37 (LCDR3) as defined by the Kabat numbering system. In some embodiments, the antibody or antigen-binding fragment comprises a heavy chain variable region comprising an amino acid sequence of SEQ ID NO: 42, and a light chain variable region comprising an amino acid sequence of SEQ ID NO: 43. In some embodiments, the antibody or antigen-binding fragment comprises an IgG1 domain.

In some embodiments, the anti-PSMA antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system. In some embodiments, the antibody or antigen-binding fragment comprises a heavy chain variable region comprising an amino acid sequence of SEQ ID NO: 14, and a light chain variable region comprising an amino acid sequence of SEQ ID NO: 19. In some embodiments, the antibody or antigen-binding fragment comprises an IgG1 domain.

In some embodiments, the anti-PSMA antibodies and antigen-binding fragments disclosed herein have favorable thermal stability. In some embodiments, the anti-PSMA antibodies or antigen-binding fragments disclosed herein have a melting temperature (Tm)>70° C., >75° C., or >80° C. In some embodiments, the anti-PSMA antibodies or antigen-binding fragments disclosed herein have a melting temperature (Tm)>80° C. In some embodiments, the anti-PSMA antibodies or antigen-binding fragments disclosed herein have a higher melting temperature (Tm) than alternative anti-PSMA antibodies, e.g., J591 and deJ591. See U.S. Pat. Nos. 11,059,903 and 7,045,605.

An anti-PSMA antibody or antigen-binding fragment may be selected to improve or retain a variety of factors, including to retain target binding affinity, enhance thermal stability, and/or minimize immunogenicity. In some embodiments, an anti-PSMA antibody is selected for exhibiting superiority in more than one category. In some embodiments, an anti-PSMA antibody is selected for exhibiting improvement in more than one category even if not necessarily the best antibody in any one category.

In some embodiments, an anti-PSMA antibody or antigen-binding fragment comprising three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system, is selected for exhibiting retained target binding affinity, enhanced thermal stability, and minimized immunogenicity compared to other anti-PSMA antibodies, e.g., J591 and/or deJ591. In some embodiments, an anti-PSMA antibody or antigen-binding fragment comprising three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system, is selected for exhibiting retained target binding affinity and minimized immunogenicity compared to other anti-PSMA antibodies, e.g., J591, deJ591, and/or anti-PSMA antibodies disclosed herein.

In some embodiments, an anti-PSMA antibody or antigen-binding fragment comprising a heavy chain variable region comprising an amino acid sequence of SEQ ID NO: 14 and a light chain variable region comprising an amino acid sequence of SEQ ID NO: 19 is selected for exhibiting retained target binding affinity, enhanced thermal stability, and minimized immunogenicity compared to other anti-PSMA antibodies, e.g., J591 and/or deJ591. In some embodiments, an anti-PSMA antibody or antigen-binding fragment comprising a heavy chain variable region comprising an amino acid sequence of SEQ ID NO: 14 and a light chain variable region comprising an amino acid sequence of SEQ ID NO: 19 is selected for exhibiting retained target binding affinity and minimized immunogenicity compared to other anti-PSMA antibodies, e.g., J591, deJ591, and/or anti-PSMA antibodies disclosed herein.

In some embodiments, the antibodies disclosed herein may comprise an IgG constant domain, e.g., an IgG1 domain or an IgG1 domain that has been modified to reduce binding to an Fc receptor, e.g., an Fcγ receptor (FcγR) as compared to a wild-type constant domain-containing (e.g., a wild-type IgG1-containing) antibody. Reduced binding to an Fc receptor, e.g., to an FcγR, can be measured as a comparison to the binding of the antibody without the modification to the same receptor. Reduced binding may be by at least about 10-fold, and preferably at least about 100-fold as compared to the antibody containing the unmodified constant domain. Reduced binding may be measured using any assay known in the art. For example, reduced binding may be measured using a fluorescence resonance energy transfer (FRET) assay.

In some embodiments, the antibodies disclosed herein may comprise an IgG constant domain, e.g., an IgG1 domain or an IgG1 domain that has been modified to increase binding to an Fc receptor, e.g., an Fcγ receptor (FcγR) as compared to a wild-type constant domain-containing (e.g., a wild-type IgG1-containing) antibody. Increased binding to an Fc receptor, e.g., to an FcγR, can be measured as a comparison to the binding of the antibody without the modification to the same receptor. Increased binding may be by at least about 5-fold, and preferably at least about 10-fold as compared to the antibody containing the unmodified constant domain. Increased binding may be measured using a fluorescence resonance energy transfer (FRET) assay. In some embodiments, the modified IgG constant domain is modified by Fc engineering and/or glycan modification, e.g., afucosylation.

In some embodiments, an antibody disclosed herein may comprise an IgG1 domain comprising the mutations L234A, L235A, P238S, H268Q, and/or K274Q (e.g., comprising all of those mutations) according to the EU numbering of Kabat. See, e.g., Wang et al. (2017) Protein Cell 9(1):63-73; Vafa et al. (2014) Methods 1;65(1):114-26; and Tam et al. (2017) Antibodies 1 6(3):12. Without being bound by theory, these mutations may reduce binding of the antibody to an Fcγ receptor (FcγR), which may reduce non-antigen mediated uptake of antibodies or ADCs by immune cells, such as neutrophils, thus reducing neutropenia. Reduced neutropenia may be measured using any assay known in the art. For example, reduced neutropenia may be measured using a flow cytometry assay.

In some embodiments, an antibody that specifically binds a PSMA protein comprises a heavy chain having an amino acid sequence selected from SEQ ID NOs: 47-60 listed in Table 8, infra and/or comprising a set of CDRs and/or a variable domain from the amino acid sequences in Table 8. In some embodiments, an antibody that specifically binds a PSMA protein comprises a light chain having an amino acid sequence selected from SEQ ID NOs: 61-66 listed in Table 8, infra and/or comprising a set of CDRs and/or a variable domain from the amino acid sequences in Table 8.

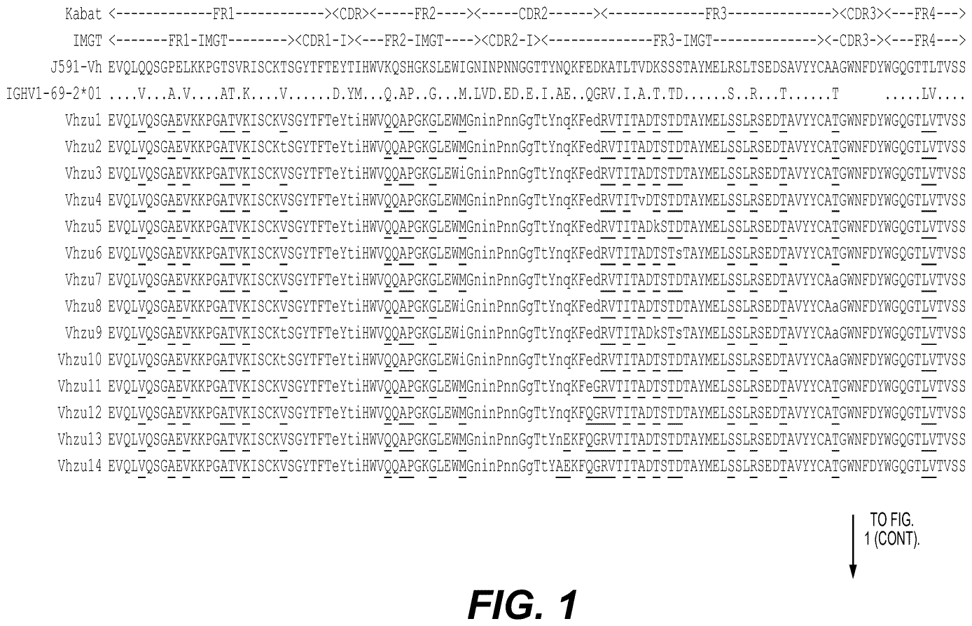

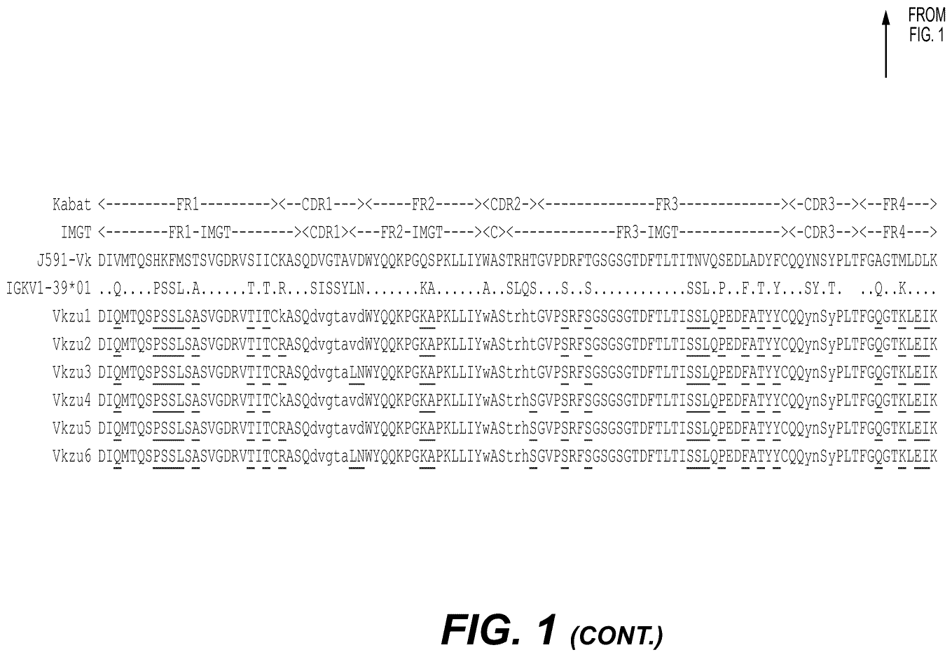

Amino acid and nucleic acid sequences of exemplary antibodies of the present disclosure are set forth in Tables 1-9. The monoclonal antibody Kabat CDR and variable region consensus sequences (Tables 1 and 2, respectively) reflect the alignment of the heavy and light chain variable region sequences represented by SEQ ID NOs: 1-20 (FIG. 1). Residues that differ between clones are represented by “X” in SEQ ID NOs: 42-46. An anti-PSMA antibody or antigen-binding fragment as described herein may be defined by the consensus CDR sequences of Table 1 in combination with the CDR sequences of Table 3, e.g., by selecting a HC CDR2, LC CDR1, and/or LCDR2 sequence of Table 1 and a HC CDR1, HC CDR2, HC CDR3, LC CDR1, LC CDR2, and/or LC CDR3 of Table 3 to describe an antibody by its three heavy chain and three light chain CDR sequences.

TABLE 1
Amino Acid Sequences of Consensus mAb Kabat CDRs

| | | |
|---|---|---|
| IgG chain | SEQ ID NO | Amino acid sequence |

Heavy chain CDRs

| | | |
|---|---|---|
| HC CDR2 | 44 | NINPNNGGTTYX¹X²KFX³X⁴ |

Light chain CDRs

| | | |
|---|---|---|
| LC CDR1 | 45 | X⁵ASQDVGTAX⁶X⁷ |
| LC CDR2 | 46 | WASTRHX⁸ |

X¹ = A or N; X² = E or Q; X³ = Q or E; X⁴ = G or D; X⁵ = R or K; X⁶ = V or L; X⁷ = D or N; and X⁸ = S or T

TABLE 2
Amino Acid Sequences of Consensus mAb Variable Regions

| IgG chain | SEQ ID NO | Amino acid sequence of variable region |
|---|---|---|
| heavy chain | 42 | EVQLVQSGAEVKKPGATVKISCKX⁹SGYTFTEYTIHWVQQAPG |
| | | KGLEWX¹⁰GNINPNNGGTTYX¹¹X¹²KFX¹³X¹⁴RVTITX¹⁵DX¹⁶ST |
| | | X¹⁷TAYMELSSLRSEDTAVYYCAX¹⁸GWNFDYWGQGTLLTVSS |
| light chain | 43 | DIQMTQSPSSLSASVGDRVTITCX¹⁹ASQDVGTAX²⁰X²¹WYQQK |
| | | PGKAPKLLIYWASTRHX²²GVPSRFSGSGSGTDFTLTISSLQP |
| | | EDFATYYCQQYNSYPLTFGQGTKLEIK |

X⁹ = V or T; X¹⁰ = M or I; X¹¹ = A or N; X¹² = E or Q; X¹³ = Q or E; X¹⁴ = G or D; X¹⁵ = A or V; X¹⁶ = T or K; X¹⁷ = D or S; X¹⁸ = T or A; X¹⁹ = R or K; X²⁰ = L or V; X²¹ = N or D; and X²² = S or T

TABLE 3
Amino Acid Sequences of mAb Kabat CDRs

| | | |
|---|---|---|
| IgG chain | SEQ ID NO | Amino acid sequence |

Heavy chain CDRs

| | | |
|---|---|---|
| HC CDR1 | 21 | EYTIH |
| HC CDR2 | 22 | NINPNNGGTTYAEKFQG |
| | 23 | NINPNNGGTTYNEKFQG |
| | 24 | NINPNNGGTTYNQKFQG |
| | 25 | NINPNNGGTTYNQKFEG |
| | 26 | NINPNNGGTTYNQKFED |
| HC CDR3 | 27 | GWNFDY |

Light chain CDRs

| | | |
|---|---|---|
| LC CDR1 | 32 | RASQDVGTAVD |
| | 33 | KASQDVGTAVD |
| | 34 | RASQDVGTALN |
| LC CDR2 | 35 | WASTRHS |
| | 36 | WASTRHT |
| LC CDR3 | 37 | QQYNSYPLT |

TABLE 4
Nucleic acid sequences encoding mAb Kabat CDRs

| | | |
|---|---|---|
| IgG chain | SEQ ID NO | Nucleic acid sequence |

Heavy chain CDRs

| | | |
|---|---|---|
| HC CDR1 | 74 | GAGTACACCATTCAC |
| HC CDR2 | 75 | AACATCAACCCCAACAACGGCGGCACCACCTACGCC |
| | | GAAAAATTCCAGGGC |
| | 76 | AACATCAACCCCAACAACGGCGGCACCACCTACAAC |
| | | GAAAAATTCCAGGGC |
| | 77 | AACATCAACCCCAACAACGGCGGCACCACCTACAAC |
| | | CAGAAATTCCAGGGC |
| | 78 | AACATCAACCCCAACAACGGCGGCACCACCTACAAC |
| | | CAGAAATTCGAGGGC |
| | 79 | AACATCAACCCCAACAACGGCGGCACCACCTACAAC |
| | | CAGAAATTCGAGGAC |
| HC CDR3 | 80 | GGCTGGAACTTCGACTAC |

Light chain CDRs

| | | |
|---|---|---|
| LC CDR1 | 81 | AGAGCCAGCCAGGATGTGGGCACCGCTGTTGAC |
| | 82 | AAGGCCAGCCAGGATGTGGGCACCGCTGTTGAC |
| | 83 | AGAGCCAGCCAGGATGTGGGCACCGCTCTGAAC |
| LC CDR2 | 84 | TGGGCCAGCACAAGACACAGC |
| | 85 | TGGGCCAGCACAAGACACACA |
| LC CDR3 | 86 | CAGCAGTACAACAGCTACCCTCTGACC |

TABLE 5
Amino acid sequences of mAb IMGT CDRs

| | | |
|---|---|---|
| IgG chain | SEQ ID NO | Amino acid sequence |

Heavy chain CDRs

| | | |
|---|---|---|
| HC CDR1 | 28 | GYTFTEYT |
| HC CDR2 | 29 | INPNNGGT |
| HC CDR3 | 30 | ATGWNFDY |
| | 31 | AAGWNFDY |

Light chain CDRs

| | | |
|---|---|---|
| LC CDR1 | 38 | QDVGTA |
| LC CDR2 | 39 | WAS |
| LC CDR3 | 37 | QQYNSYPLT |

TABLE 6
Nucleic acid sequences encoding mAb IMGT CDRs

| | | |
|---|---|---|
| IgG chain | SEQ ID NO | Nucleic acid sequence |

Heavy chain CDRs

| | | |
|---|---|---|
| HC CDR1 | 87 | GGCTACACCTTCACCGAGTACACC |
| HC CDR2 | 88 | ATCAACCCCAACAACGGCGGCACC |
| HC CDR3 | 89 | GCCACCGGCTGGAACTTCGACTAC |
| | 90 | GCCGCCGGCTGGAACTTCGACTAC |

Light chain CDRs

| | | |
|---|---|---|
| LC CDR1 | 91 | CAGGATGTGGGCACCGCT |
| LC CDR2 | 92 | TGGGCCAGC |
| LC CDR3 | 86 | CAGCAGTACAACAGCTACCCTCTGACC |

TABLE 7
Amino acid sequences of mAb variable regions

| SEQ ID NO | Name | Target | Fv | Amino acid sequence of variable region |
|---|---|---|---|---|
| 1 | VhZu1 | human | Vh | EVQLVQSGAEVKKPGATVKISCKVSGYTFTEYTI |
| | | PSMA | | HWVQQAPGKGLEWMGNINPNNGGTTYNQKFEDRV |
| | | | | TITADTSTDTAYMELSSLRSEDTAVYYCATGWNF |
| | | | | DYWGQGTLVTVSS |
| 2 | VhZu2 | human | Vh | EVQLVQSGAEVKKPGATVKISCKTSGYTFTEYTI |
| | | PSMA | | HWVQQAPGKGLEWMGNINPNNGGTTYNQKFEDRV |
| | | | | TITADTSTDTAYMELSSLRSEDTAVYYCATGWNF |
| | | | | DYWGQGTLVTVSS |
| 3 | VhZu3 | human | Vh | EVQLVQSGAEVKKPGATVKISCKVSGYTFTEYTI |
| | | PSMA | | HWVQQAPGKGLEWIGNINPNNGGTTYNQKFEDRV |
| | | | | TITADTSTDTAYMELSSLRSEDTAVYYCATGWNF |
| | | | | DYWGQGTLVTVSS |
| 4 | VhZu4 | human | Vh | EVQLVQSGAEVKKPGATVKISCKVSGYTFTEYTI |
| | | PSMA | | HWVQQAPGKGLEWMGNINPNNGGTTYNQKFEDRV |
| | | | | TITVDTSTDTAYMELSSLRSEDTAVYYCATGWNF |
| | | | | DYWGQGTLVTVSS |
| 5 | VhZu5 | human | Vh | EVQLVQSGAEVKKPGATVKISCKVSGYTFTEYTI |
| | | PSMA | | HWVQQAPGKGLEWMGNINPNNGGTTYNQKFEDRV |
| | | | | TITADKSTDTAYMELSSLRSEDTAVYYCATGWNF |
| | | | | DYWGQGTLVTVSS |
| 6 | VhZu6 | human | Vh | EVQLVQSGAEVKKPGATVKISCKVSGYTFTEYTI |
| | | PSMA | | HWVQQAPGKGLEWMGNINPNNGGTTYNQKFEDRV |
| | | | | TITADTSTSTAYMELSSLRSEDTAVYYCATGWNF |
| | | | | DYWGQGTLVTVSS |
| 7 | VhZu7 | human | Vh | EVQLVQSGAEVKKPGATVKISCKVSGYTFTEYTI |
| | | PSMA | | HWVQQAPGKGLEWMGNINPNNGGTTYNQKFEDRV |
| | | | | TITADTSTDTAYMELSSLRSEDTAVYYCAAGWNF |
| | | | | DYWGQGTLVTVSS |
| 8 | VhZu8 | human | Vh | EVQLVQSGAEVKKPGATVKISCKVSGYTFTEYTI |
| | | PSMA | | HWVQQAPGKGLEWIGNINPNNGGTTYNQKFEDRV |
| | | | | TITADTSTDTAYMELSSLRSEDTAVYYCAAGWNF |
| | | | | DYWGQGTLVTVSS |
| 9 | VhZu9 | human | Vh | EVQLVQSGAEVKKPGATVKISCKTSGYTFTEYTI |
| | | PSMA | | HWVQQAPGKGLEWIGNINPNNGGTTYNQKFEDRV |
| | | | | TITADKSTSTAYMELSSLRSEDTAVYYCAAGWNF |
| | | | | DYWGQGTLVTVSS |
| 10 | VhZu10 | human | Vh | EVQLVQSGAEVKKPGATVKISCKTSGYTFTEYTI |
| | | PSMA | | HWVQQAPGKGLEWIGNINPNNGGTTYNQKFEDRV |
| | | | | TITADTSTDTAYMELSSLRSEDTAVYYCAAGWNF |
| | | | | DYWGQGTLVTVSS |
| 11 | VhZu11 | human | Vh | EVQLVQSGAEVKKPGATVKISCKVSGYTFTEYTI |
| | | PSMA | | HWVQQAPGKGLEWMGNINPNNGGTTYNQKFEGRV |
| | | | | TITADTSTDTAYMELSSLRSEDTAVYYCATGWNF |
| | | | | DYWGQGTLVTVSS |
| 12 | VhZu12 | human | Vh | EVQLVQSGAEVKKPGATVKISCKVSGYTFTEYTI |
| | | PSMA | | HWVQQAPGKGLEWMGNINPNNGGTTYNQKFQGRV |
| | | | | TITADTSTDTAYMELSSLRSEDTAVYYCATGWNF |
| | | | | DYWGQGTLVTVSS |
| 13 | VhZu13 | human | Vh | EVQLVQSGAEVKKPGATVKISCKVSGYTFTEYTI |
| | | PSMA | | HWVQQAPGKGLEWMGNINPNNGGTTYNEKFQGRV |
| | | | | TITADTSTDTAYMELSSLRSEDTAVYYCATGWNF |
| | | | | DYWGQGTLVTVSS |
| 14 | VhZu14 | human | Vh | EVQLVQSGAEVKKPGATVKISCKVSGYTFTEYTI |
| | | PSMA | | HWVQQAPGKGLEWMGNINPNNGGTTYAEKFQGRV |
| | | | | TITADTSTDTAYMELSSLRSEDTAVYYCATGWNF |
| | | | | DYWGQGTLVTVSS |
| 15 | VkZu1 | human | Vk | DIQMTQSPSSLSASVGDRVTITCKASQDVGTAVD |
| | | PSMA | | WYQQKPGKAPKLLIYWASTRHTGVPSRFSGSGSG |
| | | | | TDFTLTISSLQPEDFATYYCQQYNSYPLTFGQGT |
| | | | | KLEIK |
| 16 | VkZu2 | human | Vk | DIQMTQSPSSLSASVGDRVTITCRASQDVGTAVD |
| | | PSMA | | WYQQKPGKAPKLLIYWASTRHTGVPSRFSGSGSG |
| | | | | TDFTLTISSLQPEDFATYYCQQYNSYPLTFGQGT |
| | | | | KLEIK |
| 17 | VkZu3 | human | Vk | DIQMTQSPSSLSASVGDRVTITCRASQDVGTALN |
| | | PSMA | | WYQQKPGKAPKLLIYWASTRHTGVPSRFSGSGSG |
| | | | | TDFTLTISSLQPEDFATYYCQQYNSYPLTFGQGT |
| | | | | KLEIK |
| 18 | VkZu4 | human | Vk | DIQMTQSPSSLSASVGDRVTITCKASQDVGTAVD |
| | | PSMA | | WYQQKPGKAPKLLIYWASTRHSGVPSRFSGSGSG |
| | | | | TDFTLTISSLQPEDFATYYCQQYNSYPLTFGQGT |
| | | | | KLEIK |
| 19 | VkZu5 | human | Vk | DIQMTQSPSSLSASVGDRVTITCRASQDVGTAVD |
| | | PSMA | | WYQQKPGKAPKLLIYWASTRHSGVPSRFSGSGSG |
| | | | | TDFTLTISSLQPEDFATYYCQQYNSYPLTFGQGT |
| | | | | KLEIK |
| 20 | VkZu6 | human | Vk | DIQMTQSPSSLSASVGDRVTITCRASQDVGTALN |
| | | PSMA | | WYQQKPGKAPKLLIYWASTRHSGVPSRFSGSGSG |
| | | | | TDFTLTISSLQPEDFATYYCQQYNSYPLTFGQGT |
| | | | | KLEIK |
| 40 | deJ591Vh | human | Vh | EVQLVQSGPEVKKPGATVKISCKTSGYTFTEYTI |
| | | PSMA | | HWVKQAPGKGLEWIGNINPNNGGTTYNQKFEDKA |
| | | | | TLTVDKSTDTAYMELSSLRSEDTAVYYCAAGWNF |
| | | | | DYWGQGTLLTVSS |
| 41 | deJ591Vk | human | Vk | DIQMTQSPSSLSTSVGDRVTLTCKASQDVGTAVD |
| | | PSMA | | WYQQKPGPSPKLLIYWASTRHTGIPSRFSGSGSG |
| | | | | TDFTLTISSLQPEDFADYYCQQYNSYPLTFGPGT |
| | | | | KVDIK |
| 72 | J591 Vh | human | Vh | EVQLQQSGPELKKPGTSVRISCKTSGYTFTEYTI |
| | | PSMA | | HWVKQSHGKSLEWIGNINPNNGGTTYNQKFEDKA |
| | | | | TLTVDKSSSTAYMELRSLTSEDSAVYYCAAGWNF |
| | | | | DYWGQGTTLTVSS |
| 73 | J591 Vk | human | Vk | DIVMTQSHKFMSTSVGDRVSIICKASQDVGTAVD |
| | | PSMA | | WYQQKPGQSPKLLIYWASTRHTGVPDRFTGSGSG |
| | | | | TDFTLTTTNVQSEDLADYFCQQYNSYPLTFGAGT |
| | | | | MLDLK |

Bolded text indicates amino acid positions corresponding to CDR sequences according to the Kabat system; underlined text indicates amino acid positions corresponding to CDR sequences according to the IMGT system. Text that is neither bolded nor underlined correspond to the framework regions.

TABLE 8
Nucleic acid sequences encoding mAb variable regions

| SEQ ID NO | Name | Target | Fv | Nucleic acid sequence of variable region |
|---|---|---|---|---|
| 93 | VhZu1 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 94 | VhZu2 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GACCTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 95 | VhZu3 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATCGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 96 | VhZu4 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGTGGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 97 | VhZu5 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGCCGACAAGAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 98 | VhZu6 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGCCGACACCAGCACCAGCACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 99 | VhZu7 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCGCCGGCTGGAACTTC |
| | | | | GACTACTGGGGACAGGGCACACTGGTCACCGTTA |
| | | | | GCTCT |
| 100 | VhZu8 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATCGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCGCCGGCTGGAACTTC |
| | | | | GACTACTGGGGACAGGGCACACTGGTCACCGTTA |
| | | | | GCTCT |
| 101 | VhZu9 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GACCTCCGGCTACACCTTCACCGAGTACACCATT |
| | | | | CACTGGGTGCAGCAGGCCCCTGGCAAAGGACTGG |
| | | | | AATGGATCGGCAACATCAACCCCAACAACGGCGG |
| | | | | CACCACCTACAACCAGAAATTCGAGGACAGAGTG |
| | | | | ACCATCACCGCCGACAAGAGCACCAGCACAGCCT |
| | | | | ACATGGAACTGAGCAGCCTGAGAAGCGAGGACAC |
| | | | | CGCCGTGTATTATTGTGCCGCCGGCTGGAACTTC |
| | | | | GACTACTGGGGACAGGGCACACTGGTCACCGTTA |
| | | | | GCTCT |
| 102 | VhZu10 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GACCTCCGGCTACACCTTCACCGAGTACACCATT |
| | | | | CACTGGGTGCAGCAGGCCCCTGGCAAAGGACTGG |
| | | | | AATGGATCGGCAACATCAACCCCAACAACGGCGG |
| | | | | CACCACCTACAACCAGAAATTCGAGGACAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCGCCGGCTGGAACTTC |
| | | | | GACTACTGGGGACAGGGCACACTGGTCACCGTTA |
| | | | | GCTCT |
| 103 | VhZu11 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCGAGGGCAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 104 | VhZu12 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACCAGAAATTCCAGGGCAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 105 | VhZu13 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACAACGAAAAATTCCAGGGCAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 106 | VhZu14 | human | Vh | GAAGTTCAACTTGTTCAGAGCGGAGCCGAAGTGA |
| | | PSMA | | AGAAACCCGGCGCTACAGTGAAGATCAGCTGCAA |
| | | | | GGTGTCCGGCTACACCTTCACCGAGTACACCATT |
| | | | | CACTGGGTGCAGCAGGCCCCTGGCAAAGGACTGG |
| | | | | AATGGATGGGCAACATCAACCCCAACAACGGCGG |
| | | | | CACCACCTACGCCGAAAAATTCCAGGGCAGAGTG |
| | | | | ACCATCACCGCCGACACCAGCACCGATACAGCCT |
| | | | | ACATGGAACTGAGCAGCCTGAGAAGCGAGGACAC |
| | | | | CGCCGTGTATTATTGTGCCACCGGCTGGAACTTC |
| | | | | GACTACTGGGGACAGGGCACACTGGTCACCGTTA |
| | | | | GCTCT |
| 107 | VkZu1 | human | Vk | GATATTCAGATGACACAGAGCCCTAGCAGCCTGA |
| | | PSMA | | GCGCCTCTGTGGGCGATAGAGTGACAATCACATG |
| | | | | CAAGGCCAGCCAGGATGTGGGCACCGCTGTTGAC |
| | | | | TGGTATCAGCAGAAGCCTGGCAAGGCCCCTAAGC |
| | | | | TGCTGATCTACTGGGCCAGCACAAGACACACAGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 108 | VkZu2 | human | Vk | GATATTCAGATGACACAGAGCCCTAGCAGCCTGA |
| | | PSMA | | GCGCCTCTGTGGGCGATAGAGTGACAATCACATG |
| | | | | CAGAGCCAGCCAGGATGTGGGCACCGCTGTTGAC |
| | | | | TGGTATCAGCAGAAGCCTGGCAAGGCCCCTAAGC |
| | | | | TGCTGATCTACTGGGCCAGCACAAGACACACAGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 109 | VkZu3 | human | Vk | GATATTCAGATGACACAGAGCCCTAGCAGCCTGA |
| | | PSMA | | GCGCCTCTGTGGGCGATAGAGTGACAATCACATG |
| | | | | CAGAGCCAGCCAGGATGTGGGCACCGCTCTGAAC |
| | | | | TGGTATCAGCAGAAGCCTGGCAAGGCCCCTAAGC |
| | | | | TGCTGATCTACTGGGCCAGCACAAGACACACAGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 110 | VkZu4 | human | Vk | GATATTCAGATGACACAGAGCCCTAGCAGCCTGA |
| | | PSMA | | GCGCCTCTGTGGGCGATAGAGTGACAATCACATG |
| | | | | CAAGGCCAGCCAGGATGTGGGCACCGCTGTTGAC |
| | | | | TGGTATCAGCAGAAGCCTGGCAAGGCCCCTAAGC |
| | | | | TGCTGATCTACTGGGCCAGCACAAGACACAGCGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 111 | VkZu5 | human | Vk | GATATTCAGATGACACAGAGCCCTAGCAGCCTGA |
| | | PSMA | | GCGCCTCTGTGGGCGATAGAGTGACAATCACATG |
| | | | | CAGAGCCAGCCAGGATGTGGGCACCGCTGTTGAC |
| | | | | TGGTATCAGCAGAAGCCTGGCAAGGCCCCTAAGC |
| | | | | TGCTGATCTACTGGGCCAGCACAAGACACAGCGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 112 | VkZu6 | human | Vk | GATATTCAGATGACACAGAGCCCTAGCAGCCTGA |
| | | PSMA | | GCGCCTCTGTGGGCGATAGAGTGACAATCACATG |
| | | | | CAGAGCCAGCCAGGATGTGGGCACCGCTCTGAAC |
| | | | | TGGTATCAGCAGAAGCCTGGCAAGGCCCCTAAGC |
| | | | | TGCTGATCTACTGGGCCAGCACAAGACACAGCGG |
| | | | | CGTGCCAAGCAGATTTTCTGGCAGCGGCTCTGGC |
| | | | | ACCGACTTCACCCTGACCATTTCTAGCCTCCAGC |
| | | | | CTGAGGACTTCGCCACCTACTACTGTCAGCAGTA |
| | | | | CAACAGCTACCCTCTGACCTTTGGCCAGGGCACC |
| | | | | AAGCTGGAAATCAAA |
| 113 | deJ591Vh | human | Vh | GAAGTTCAACTTGTGCAGAGCGGCCCTGAAGTGA |
| | | PSMA | | AGAAACCTGGCGCTACCGTGAAGATCAGCTGCAA |
| | | | | GACCAGCGGCTACACCTTCACCGAGTACACCATC |
| | | | | CACTGGGTTAAGCAGGCCCCTGGCAAAGGCCTGG |
| | | | | AATGGATCGGCAACATCAACCCCAACAACGGCGG |
| | | | | CACCACCTACAACCAGAAGTTCGAGGATAAGGCC |
| | | | | ACACTGACCGTGGACAAGAGCACCGACACAGCCT |
| | | | | ACATGGAACTGAGCAGCCTGAGAAGCGAGGACAC |
| | | | | CGCCGTGTATTATTGTGCCGCCGGATGGAACTTC |
| | | | | GACTACTGGGGACAGGGCACCCTGCTGACAGTCT |
| | | | | CTTCT |
| 114 | deJ591Vk | human | Vk | GATATTCAGATGACACAGAGCCCCAGCAGCCTGA |
| | | PSMA | | GCACATCTGTGGGAGATAGAGTGACCCTGACCTG |
| | | | | CAAGGCCTCTCAGGATGTGGGAACCGCCGTGGAC |
| | | | | TGGTATCAGCAGAAACCCGGACCTTCTCCAAAGC |
| | | | | TGCTGATCTACTGGGCCAGCACCAGACACACAGG |
| | | | | CATCCCTAGCAGATTTTCTGGCAGCGGCTCTGGC |
| | | | | ACCGACTTCACCCTGACCATTTCTAGCCTCCAGC |
| | | | | CTGAGGACTTCGCCGACTACTACTGCCAGCAGTA |
| | | | | CAACAGCTACCCTCTGACATTCGGCCCTGGCACC |
| | | | | AAGGTGGACATCAAA |
| 139 | J591 Vh | human | Vh | GAAGTTCAACTTCAGCAGTCTGGCCCCGAGCTGA |
| | | PSMA | | AGAAACCTGGAACAAGCGTGCGGATCAGCTGCAA |
| | | | | GACCAGCGGCTACACCTTCACCGAGTACACCATC |
| | | | | CACTGGGTCAAGCAGAGCCACGGCAAGTCTCTGG |
| | | | | AATGGATCGGCAACATCAACCCCAACAACGGCGG |
| | | | | CACCACCTACAACCAGAAGTTCGAGGATAAGGCC |
| | | | | ACACTGACCGTGGACAAGAGCAGCAGCACAGCCT |
| | | | | ACATGGAACTGAGAAGCCTGACCAGCGAGGACAG |
| | | | | CGCCGTGTATTATTGTGCCGCCGGATGGAACTTC |
| | | | | GACTACTGGGGACAGGGCACAACCCTGACCGTTA |
| | | | | GCTCT |
| 140 | J591 Vk | human | Vk | GATATTGTGATGACCCAGAGCCACAAGTTCATGA |
| | | PSMA | | GCACCAGCGTGGGCGACAGAGTGTCCATCATCTG |
| | | | | CAAGGCCTCTCAGGATGTGGGCACCGCCGTTGAC |
| | | | | TGGTATCAGCAGAAACCTGGACAGAGCCCCAAGC |
| | | | | TGCTGATCTACTGGGCCTCTACAAGACACACAGG |
| | | | | CGTGCCCGATAGATTCACAGGCTCTGGCAGCGGC |
| | | | | ACCGACTTTACCCTGACCATCACAAACGTGCAGA |
| | | | | GCGAGGACCTGGCCGACTACTTCTGCCAGCAGTA |
| | | | | CAACAGCTACCCTCTGACCTTTGGAGCCGGCACC |
| | | | | ATGCTGGATCTTAAA |

TABLE 9
Amino acid sequences of full-length mAb Ig chains

| SEQ | | | | Amino acid sequence of full-length |
|---|---|---|---|---|
| ID NO | Name | Target | Ig chain | mAb Igc hain |
| 47 | zuJ591-H1 | human | Human | EVQLVQSGAEVKKPGATVKISCKVSGYTFTEYT |
| | | PSMA | IgG1 | IHWVQQAPGKGLEWMGNINPNNGGTTYNQKFED |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 48 | zuJ591-H2 | human | Human | EVQLVQSGAEVKKPGATVKISCKTSGYTFTEYT |
| | | PSMA | IgG1 | IHWVQQAPGKGLEWMGNINPNNGGTTYNQKFED |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 49 | zuJ591-H3 | human | Human | EVQLVQSGAEVKKPGATVKISCKVSGYTFTEYT |
| | | PSMA | IgG1 | IHWVQQAPGKGLEWIGNINPNNGGTTYNQKFED |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 50 | zuJ591-H4 | human | Human | EVQLVQSGAEVKKPGATVKISCKVSGYTFTEYT |
| | | PSMA | IgG1 | IHWVQQAPGKGLEWMGNINPNNGGTTYNQKFED |
| | | | | RVTITVDTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 51 | zuJ591-H5 | human | Human | EVQLVQSGAEVKKPGATVKISCKVSGYTFTEYT |
| | | PSMA | IgG1 | IHWVQQAPGKGLEWMGNINPNNGGTTYNQKFED |
| | | | | RVTITADKSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 52 | zuJ591-H6 | human | Human | EVQLVQSGAEVKKPGATVKISCKVSGYTFTEYT |
| | | PSMA | IgG1 | IHWVQQAPGKGLEWMGNINPNNGGTTYNQKFED |
| | | | | RVTITADTSTSTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 53 | zuJ591-H7 | human | Human | EVQLVQSGAEVKKPGATVKISCKVSGYTFTEYT |
| | | PSMA | IgG1 | IHWVQQAPGKGLEWMGNINPNNGGTTYNQKFED |
| | | | | RVTITADTSTDTAYMELSSLRSEDTAVYYCAAG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 54 | zuJ591-H8 | human | Human | EVQLVQSGAEVKKPGATVKISCKVSGYTFTEYT |
| | | PSMA | IgG1 | IHWVQQAPGKGLEWIGNINPNNGGTTYNQKFED |
| | | | | RVTITADTSTDTAYMELSSLRSEDTAVYYCAAG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 55 | zuJ591-H9 | human | Human | EVQLVQSGAEVKKPGATVKISCKTSGYTFTEYT |
| | | PSMA | IgG1 | IHWVQQAPGKGLEWIGNINPNNGGTTYNQKFED |
| | | | | RVTITADKSTSTAYMELSSLRSEDTAVYYCAAG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 56 | zuJ591-H10 | human | Human | EVQLVQSGAEVKKPGATVKISCKTSGYTFTEYT |
| | | PSMA | IgG1 | IHWVQQAPGKGLEWIGNINPNNGGTTYNQKFED |
| | | | | RVTITADTSTDTAYMELSSLRSEDTAVYYCAAG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 57 | zuJ591-H11 | human | Human | EVQLVQSGAEVKKPGATVKISCKVSGYTFTEYT |
| | | PSMA | IgG1 | IHWVQQAPGKGLEWMGNINPNNGGTTYNQKFEG |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 58 | zuJ591-H12 | human | Human | EVQLVQSGAEVKKPGATVKISCKVSGYTFTEYT |
| | | PSMA | IgG1 | IHWVQQAPGKGLEWMGNINPNNGGTTYNQKFQG |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 59 | zuJ591-H13 | human | Human | EVQLVQSGAEVKKPGATVKISCKVSGYTFTEYT |
| | | PSMA | IgG1 | IHWVQQAPGKGLEWMGNINPNNGGTTYNEKFQG |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 60 | zuJ591-H14 | human | Human | EVQLVQSGAEVKKPGATVKISCKVSGYTFTEYT |
| | | PSMA | IgG1 | IHWVQQAPGKGLEWMGNINPNNGGTTYAEKFQG |
| | | | | RVTITADTSTDTAYMELSSLRSEDTAVYYCATG |
| | | | | WNFDYWGQGTLVTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 61 | zuJ591-L1 | human | Human | DIQMTQSPSSLSASVGDRVTITCKASQDVGTAV |
| | | PSMA | IgK | DWYQQKPGKAPKLLIYWASTRHTGVPSRFSGSG |
| | | | | SGTDFTLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 62 | zuJ591-L2 | human | Human | DIQMTQSPSSLSASVGDRVTITCRASQDVGTAV |
| | | PSMA | IgK | DWYQQKPGKAPKLLIYWASTRHTGVPSRFSGSG |
| | | | | SGTDFTLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 63 | zuJ591-L3 | human | Human | DIQMTQSPSSLSASVGDRVTITCRASQDVGTAL |
| | | PSMA | IgK | NWYQQKPGKAPKLLIYWASTRHTGVPSRFSGSG |
| | | | | SGTDFTLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 64 | zuJ591-L4 | human | Human | DIQMTQSPSSLSASVGDRVTITCKASQDVGTAV |
| | | PSMA | IgK | DWYQQKPGKAPKLLIYWASTRHSGVPSRFSGSG |
| | | | | SGTDFTLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 65 | zuJ591-L5 | human | Human | DIQMTQSPSSLSASVGDRVTITCRASQDVGTAV |
| | | PSMA | IgK | DWYQQKPGKAPKLLIYWASTRHSGVPSRFSGSG |
| | | | | SGTDETLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 66 | zuJ591-L6 | human | Human | DIQMTQSPSSLSASVGDRVTITCRASQDVGTAL |
| | | PSMA | IgK | NWYQQKPGKAPKLLIYWASTRHSGVPSRFSGSG |
| | | | | SGTDFTLTISSLQPEDFATYYCQQYNSYPLTFG |
| | | | | QGTKLEIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |
| 115 | deJ591-HC | human | Human | EVQLVQSGPEVKKPGATVKISCKTSGYTFTEYT |
| | | PSMA | IgG1 | IHWVKQAPGKGLEWIGNINPNNGGTTYNQKFED |
| | | | | KATLTVDKSTDTAYMELSSLRSEDTAVYYCAAG |
| | | | | WNFDYWGQGTLLTVSSASTKGPSVFPLAPSSKS |
| | | | | TSGGTAALGCLVKDYFPEPVTVSWNSGALTSGV |
| | | | | HTFPAVLQSSGLYSLSSVVTVPSSSLGTQTYIC |
| | | | | NVNHKPSNTKVDKKVEPKSCDKTHTCPPCPAPE |
| | | | | LLGGPSVFLFPPKPKDTLMISRTPEVTCVVVDV |
| | | | | SHEDPEVKFNWYVDGVEVHNAKTKPREEQYNST |
| | | | | YRVVSVLTVLHQDWLNGKEYKCKVSNKALPAPI |
| | | | | EKTISKAKGQPREPQVYTLPPSRDELTKNQVSL |
| | | | | TCLVKGFYPSDIAVEWESNGQPENNYKTTPPVL |
| | | | | DSDGSFFLYSKLTVDKSRWQQGNVFSCSVMHEA |
| | | | | LHNHYTQKSLSLSPGK |
| 116 | deJ591-LC | human | Human | DIQMTQSPSSLSTSVGDRVTLTCKASQDVGTAV |
| | | PSMA | IgK | DWYQQKPGPSPKLLIYWASTRHTGIPSRFSGSG |
| | | | | SGTDFTLTISSLQPEDFADYYCQQYNSYPLTFG |
| | | | | PGTKVDIKRTVAAPSVFIFPPSDEQLKSGTASV |
| | | | | VCLLNNFYPREAKVQWKVDNALQSGNSQESVTE |
| | | | | QDSKDSTYSLSSTLTLSKADYEKHKVYACEVTH |
| | | | | QGLSSPVTKSFNRGEC |

Bolded text indicates amino acid positions corresponding to CDR sequences according to the Kabat system; underlined text indicates amino acid positions corresponding to CDR sequences according to the IMGT system. Text that is neither bolded nor underlined correspond to the framework regions.

TABLE 10
Nucleic acid sequences of full-length mAb Ig chains

| SEQ | | | | Amino acid sequence of full-length |
|---|---|---|---|---|
| ID NO | Name | Target | Ig chain | mAb Ig chain |
| 117 | zuJ591-H1 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 118 | zuJ591-H2 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGACCTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 119 | zuJ591-H3 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATCGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 120 | zuJ591-H4 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGTGGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 121 | zuJ591-H5 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGCCGACAAGAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 122 | zuJ591-H6 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGCCGACACCAGCACCAGC |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 123 | zuJ591-H7 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCGCCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 124 | zuJ591-H8 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATCGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCGCCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 125 | zuJ591-H9 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGACCTCCGGCTACACCTTCACCGAGTACACC |
| | | | | ATTCACTGGGTGCAGCAGGCCCCTGGCAAAGGA |
| | | | | CTGGAATGGATCGGCAACATCAACCCCAACAAC |
| | | | | GGCGGCACCACCTACAACCAGAAATTCGAGGAC |
| | | | | AGAGTGACCATCACCGCCGACAAGAGCACCAGC |
| | | | | ACAGCCTACATGGAACTGAGCAGCCTGAGAAGC |
| | | | | GAGGACACCGCCGTGTATTATTGTGCCGCCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| I126 | zuJ591-H10 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGACCTCCGGCTACACCTTCACCGAGTACACC |
| | | | | ATTCACTGGGTGCAGCAGGCCCCTGGCAAAGGA |
| | | | | CTGGAATGGATCGGCAACATCAACCCCAACAAC |
| | | | | GGCGGCACCACCTACAACCAGAAATTCGAGGAC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCGCCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 127 | zuJ591-H11 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCGAGGGC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 128 | zuJ591-H12 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACCAGAAATTCCAGGGC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 129 | zuJ591-H13 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACAACGAAAAATTCCAGGGC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 130 | zuJ591-H14 | human | Human | GAAGTTCAACTTGTTCAGAGCGGAGCCGAAGTG |
| | | PSMA | IgG1 | AAGAAACCCGGCGCTACAGTGAAGATCAGCTGC |
| | | | | AAGGTGTCCGGCTACACCTTCACCGAGTACACC |
| | | | | ATTCACTGGGTGCAGCAGGCCCCTGGCAAAGGA |
| | | | | CTGGAATGGATGGGCAACATCAACCCCAACAAC |
| | | | | GGCGGCACCACCTACGCCGAAAAATTCCAGGGC |
| | | | | AGAGTGACCATCACCGCCGACACCAGCACCGAT |
| | | | | ACAGCCTACATGGAACTGAGCAGCCTGAGAAGC |
| | | | | GAGGACACCGCCGTGTATTATTGTGCCACCGGC |
| | | | | TGGAACTTCGACTACTGGGGACAGGGCACACTG |
| | | | | GTCACCGTTAGCTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 131 | zuJ591-L1 | human | Human | GATATTCAGATGACACAGAGCCCTAGCAGCCTG |
| | | PSMA | IgK | AGCGCCTCTGTGGGCGATAGAGTGACAATCACA |
| | | | | TGCAAGGCCAGCCAGGATGTGGGCACCGCTGTT |
| | | | | GACTGGTATCAGCAGAAGCCTGGCAAGGCCCCT |
| | | | | AAGCTGCTGATCTACTGGGCCAGCACAAGACAC |
| | | | | ACAGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 132 | zuJ591-L2 | human | Human | GATATTCAGATGACACAGAGCCCTAGCAGCCTG |
| | | PSMA | IgK | AGCGCCTCTGTGGGCGATAGAGTGACAATCACA |
| | | | | TGCAGAGCCAGCCAGGATGTGGGCACCGCTGTT |
| | | | | GACTGGTATCAGCAGAAGCCTGGCAAGGCCCCT |
| | | | | AAGCTGCTGATCTACTGGGCCAGCACAAGACAC |
| | | | | ACAGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 133 | zuJ591-L3 | human | Human | GATATTCAGATGACACAGAGCCCTAGCAGCCTG |
| | | PSMA | IgK | AGCGCCTCTGTGGGCGATAGAGTGACAATCACA |
| | | | | TGCAGAGCCAGCCAGGATGTGGGCACCGCTCTG |
| | | | | AACTGGTATCAGCAGAAGCCTGGCAAGGCCCCT |
| | | | | AAGCTGCTGATCTACTGGGCCAGCACAAGACAC |
| | | | | ACAGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 134 | zuJ591-L4 | human | Human | GATATTCAGATGACACAGAGCCCTAGCAGCCTG |
| | | PSMA | IgK | AGCGCCTCTGTGGGCGATAGAGTGACAATCACA |
| | | | | TGCAAGGCCAGCCAGGATGTGGGCACCGCTGTT |
| | | | | GACTGGTATCAGCAGAAGCCTGGCAAGGCCCCT |
| | | | | AAGCTGCTGATCTACTGGGCCAGCACAAGACAC |
| | | | | AGCGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 135 | zuJ591-L5 | human | Human | GATATTCAGATGACACAGAGCCCTAGCAGCCTG |
| | | PSMA | IgK | AGCGCCTCTGTGGGCGATAGAGTGACAATCACA |
| | | | | TGCAGAGCCAGCCAGGATGTGGGCACCGCTGTT |
| | | | | GACTGGTATCAGCAGAAGCCTGGCAAGGCCCCT |
| | | | | AAGCTGCTGATCTACTGGGCCAGCACAAGACAC |
| | | | | AGCGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 136 | zuJ591-L6 | human | Human | GATATTCAGATGACACAGAGCCCTAGCAGCCTG |
| | | PSMA | IgK | AGCGCCTCTGTGGGCGATAGAGTGACAATCACA |
| | | | | TGCAGAGCCAGCCAGGATGTGGGCACCGCTCTG |
| | | | | AACTGGTATCAGCAGAAGCCTGGCAAGGCCCCT |
| | | | | AAGCTGCTGATCTACTGGGCCAGCACAAGACAC |
| | | | | AGCGGCGTGCCAAGCAGATTTTCTGGCAGCGGC |
| | | | | TCTGGCACCGACTTCACCCTGACCATTTCTAGC |
| | | | | CTCCAGCCTGAGGACTTCGCCACCTACTACTGT |
| | | | | CAGCAGTACAACAGCTACCCTCTGACCTTTGGC |
| | | | | CAGGGCACCAAGCTGGAAATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |
| 137 | deJ591-HC | human | Human | GAAGTTCAACTTGTGCAGAGCGGCCCTGAAGTG |
| | | PSMA | IgG1 | AAGAAACCTGGCGCTACCGTGAAGATCAGCTGC |
| | | | | AAGACCAGCGGCTACACCTTCACCGAGTACACC |
| | | | | ATCCACTGGGTTAAGCAGGCCCCTGGCAAAGGC |
| | | | | CTGGAATGGATCGGCAACATCAACCCCAACAAC |
| | | | | GGCGGCACCACCTACAACCAGAAGTTCGAGGAT |
| | | | | AAGGCCACACTGACCGTGGACAAGAGCACCGAC |
| | | | | ACAGCCTACATGGAACTGAGCAGCCTGAGAAGC |
| | | | | GAGGACACCGCCGTGTATTATTGTGCCGCCGGA |
| | | | | TGGAACTTCGACTACTGGGGACAGGGCACCCTG |
| | | | | CTGACAGTCTCTTCTGCATCCACCAAGGGCCCA |
| | | | | TCGGTCTTCCCCCTGGCACCCTCCTCCAAGAGC |
| | | | | ACCTCTGGGGGCACAGCGGCCCTGGGCTGCCTG |
| | | | | GTCAAGGACTACTTCCCCGAACCGGTGACGGTG |
| | | | | TCGTGGAACTCAGGCGCCCTGACCAGCGGCGTG |
| | | | | CACACCTTCCCGGCTGTCCTACAGTCCTCAGGA |
| | | | | CTCTACTCCCTCAGCAGCGTGGTGACCGTGCCC |
| | | | | TCCAGCAGCTTGGGCACCCAGACCTACATCTGC |
| | | | | AACGTGAATCACAAGCCCAGCAACACCAAGGTG |
| | | | | GACAAGAAAGTTGAGCCCAAATCTTGTGACAAA |
| | | | | ACTCACACATGCCCACCGTGCCCAGCACCTGAA |
| | | | | CTCCTGGGGGGACCGTCAGTCTTCCTCTTCCCC |
| | | | | CCAAAACCCAAGGACACCCTCATGATCTCCCGG |
| | | | | ACCCCTGAGGTCACATGCGTGGTGGTGGACGTG |
| | | | | AGCCACGAAGACCCTGAGGTCAAGTTCAACTGG |
| | | | | TACGTGGACGGCGTGGAGGTGCATAATGCCAAG |
| | | | | ACAAAGCCGCGGGAGGAGCAGTACAACAGCACG |
| | | | | TACCGTGTGGTCAGCGTCCTCACCGTCCTGCAC |
| | | | | CAGGACTGGCTGAATGGCAAGGAGTACAAGTGC |
| | | | | AAGGTCTCCAACAAAGCCCTCCCAGCCCCCATC |
| | | | | GAGAAAACCATCTCCAAAGCCAAAGGGCAGCCC |
| | | | | CGAGAACCACAGGTGTACACCCTGCCCCCATCC |
| | | | | CGGGATGAGCTGACCAAGAACCAGGTCAGCCTG |
| | | | | ACCTGCCTGGTCAAAGGCTTCTATCCCAGCGAC |
| | | | | ATCGCCGTGGAGTGGGAGAGCAATGGGCAGCCG |
| | | | | GAGAACAACTACAAGACCACGCCTCCCGTGCTG |
| | | | | GACTCCGACGGCTCCTTCTTCTTATATTCAAAG |
| | | | | CTCACCGTGGACAAGAGCAGGTGGCAGCAGGGG |
| | | | | AACGTCTTCTCATGCTCCGTGATGCATGAGGCT |
| | | | | CTGCACAACCACTACACGCAGAAGAGCCTCTCC |
| | | | | CTGTCTCCCGGGAAATGA |
| 138 | deJ591-LC | human | Human | GATATTCAGATGACACAGAGCCCCAGCAGCCTG |
| | | PSMA | IgK | AGCACATCTGTGGGAGATAGAGTGACCCTGACC |
| | | | | TGCAAGGCCTCTCAGGATGTGGGAACCGCCGTG |
| | | | | GACTGGTATCAGCAGAAACCCGGACCTTCTCCA |
| | | | | AAGCTGCTGATCTACTGGGCCAGCACCAGACAC |
| | | | | ACAGGCATCCCTAGCAGATTTTCTGGCAGCGGC |
| | | | | TCTGGCACCGACTTCACCCTGACCATTTCTAGC |
| | | | | CTCCAGCCTGAGGACTTCGCCGACTACTACTGC |
| | | | | CAGCAGTACAACAGCTACCCTCTGACATTCGGC |
| | | | | CCTGGCACCAAGGTGGACATCAAACGAACTGTG |
| | | | | GCTGCACCATCTGTCTTCATCTTCCCGCCATCT |
| | | | | GATGAGCAGTTGAAATCTGGAACTGCCTCTGTT |
| | | | | GTGTGCCTGCTGAATAACTTCTATCCCAGAGAG |
| | | | | GCCAAAGTACAGTGGAAGGTGGATAACGCCCTC |
| | | | | CAATCGGGTAACTCCCAGGAGAGTGTCACAGAG |
| | | | | CAGGACAGCAAGGACAGCACCTACAGCCTCAGC |
| | | | | AGCACCCTGACGCTGAGCAAAGCAGACTACGAG |
| | | | | AAACACAAAGTCTACGCCTGCGAAGTCACCCAT |
| | | | | CAGGGCCTGAGCTCGCCCGTCACAAAGAGCTTC |
| | | | | AACAGGGGAGAGTGTTGA |

The anti-PSMA antibodies or antigen-binding fragments provided by SEQ ID NOs: 1-39 and 42-46 may provide improved properties compared to other anti-PSMA antibodies, e.g., J591 and/or deJ591. In some embodiments, the antd-PSMA antibodies or antigen-binding fragments disclosed herein have superior stability compared to other anti-PSMA antibodies, e.g., J591 and/or deJ591. In some embodiments, the antd-PSMA antibodies or antigen-binding fragments disclosed herein are less immunogenic compared to other anti-PSMA antibodies, e.g., J591 and/or deJ591.

In some embodiments, the sequences of the heavy chain variable domains, light chain variable domains, full-length heavy chains, and full-length light chains may be “mixed and matched” to create variants of the anti-PSMA antibodies. Such “mixed and matched” anti-PSMA antibodies can be tested using binding assays known in the art (e.g., ELISAs and other assays described in the Examples). In various embodiments, the antibodies disclosed herein may comprise any set of heavy and light chain variable domains listed in the tables above, or the set of six CDR sequences from the heavy and light chain set. In some embodiments, the antibodies further comprise human heavy and light chain constant domains or fragments thereof. In various embodiments, the antibodies may comprise any set of full-length heavy chain and full-length light chain sequences listed in the tables above. In some embodiments, the antibodies may comprise a human IgG heavy chain constant domain and a human kappa light chain constant domain. In some embodiments, the antibodies may comprise a human IgG1, IgG2, IgG3, or IgG4 heavy chain constant domain. In various embodiments, an antibody of the present invention comprises a human immunoglobulin G subtype 1 (IgG1) heavy chain constant domain with a human Ig kappa light chain constant domain. In some embodiments, the constant domain is a modified version of a human constant domain, e.g., comprising one or more of L234A, L235A, P238S, H268Q, and/or K274Q modifications of a human IgG1 heavy chain constant domain.

In some embodiments, an anti-PSMA antibody or antigen-binding fragment thereof provided herein comprises a heavy chain CDR2 (HCDR2) comprising SEQ ID NO: 44, wherein SEQ ID NO: 44 comprises NINPNNGGTTYX¹X²KFX³X⁴, as defined by the Kabat numbering system. In some embodiments, in SEQ ID NO: 44, X¹ is A or N, X² is E or Q, X³ is Q or E, and X⁴ is G or D. In some embodiments, in SEQ ID NO: 44, X¹ is A, X² is E, X³ is Q, and/or X⁴ is G. In some embodiments, in SEQ ID NO: 44, X¹ is N, X² is E, X³ is Q, and/or X⁴ is G. In some embodiments, in SEQ ID NO: 44, X¹ is N, X² is Q, X³ is Q, and/or X⁴ is G. In some embodiments, in SEQ ID NO: 44, X¹ is N, X² is Q, X³ is E, and/or X⁴ is G. In some embodiments, in SEQ ID NO: 44, X¹ is N, X² is Q, X³ is E, and/or X⁴ is D.

In some embodiments, an anti-PSMA antibody or antigen-binding fragment thereof provided herein comprises a light chain CDR1 (LCDR1) comprising SEQ ID NO: 45, wherein SEQ ID NO: 45 comprises X⁵ASQDVGTAX⁶X⁷, as defined by the Kabat numbering system. In some embodiments, in SEQ ID NO: 45, X⁵ is R or K, X⁶ is V or L, X⁷ is D or N. In some embodiments, in SEQ ID NO: 45, X⁵ is R, X⁶ is V, and/or X⁷ is D. In some embodiments, in SEQ ID NO: 45, X⁵ is K, X⁶ is V, and/or X⁷ is D. In some embodiments, in SEQ ID NO: 45, X⁵ is R, X⁶ is L, and/or X⁷ is N.

In some embodiments, an anti-PSMA antibody or antigen-binding fragment thereof provided herein comprises a light chain CDR2 (LCDR2) comprising SEQ ID NO: 46, wherein SEQ ID NO: 46 comprises WASTRHX⁸, as defined by the Kabat numbering system. In some embodiments, in SEQ ID NO: 46, X⁸ is S or T. In some embodiments, in SEQ ID NO: 46, X⁸ is S. In some embodiments, in SEQ ID NO: 46, X⁸ is T.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 44, heavy chain CDR3 (HCDR3) comprising SEQ ID NO: 27; light chain CDR1 (LCDR1) comprising SEQ ID NO: 45, light chain CDR2 (LCDR2) comprising SEQ ID NO: 46, and light chain CDR3 (LCDR3) comprising SEQ ID NO: 37, as defined by the Kabat numbering system. In some embodiments, in SEQ ID NO: 44, X¹ is A, X² is E, X³ is Q, and/or X⁴ is G. In some embodiments, in SEQ ID NO: 44, X¹ is N, X² is E, X³ is Q, and/or X⁴ is G. In some embodiments, in SEQ ID NO: 44, X¹ is N, X² is Q, X³ is Q and/or X⁴ is G. In some embodiments, in SEQ ID NO: 44, X¹ is N, X² is Q, X³ is E, and/or X⁴ is G. In some embodiments, in SEQ ID NO: 44, X¹ is N, X² is Q, X³ is E, and/or X⁴ is D. In some embodiments, in SEQ ID NO: 45, X⁵ is R, X⁶ is V, and/or X⁷ is D. In some embodiments, in SEQ ID NO: 45, X⁵ is K, X⁶ is V, and/or X⁷ is D. In some embodiments, in SEQ ID NO: 45, X⁵ is R, X⁶ is L, and/or X⁷ is N. In some embodiments, in SEQ ID NO: 46, X⁸ is S. In some embodiments, in SEQ ID NO: 46, X⁸ is T.

In some embodiments, in SEQ ID NO: 44, X¹ is A, X² is E, X³ is Q, and X⁴ is G; in SEQ ID NO: 45, X⁵ is R, X⁶ is V, and X⁷ is D; and in SEQ ID NO: 46, X⁸ is S. In some embodiments, in SEQ ID NO: 44, X¹ is A, X² is E, X³ is Q, and X⁴ is G; in SEQ ID NO: 45, X⁵ is R, X⁶ is V, and X⁷ is D; and in SEQ ID NO: 46, X⁸ is T. In some embodiments, in SEQ ID NO: 44, X¹ is A, X² is E, X³ is Q, and X⁴ is G; in SEQ ID NO: 45, X⁵ is K, X⁶ is V, and X⁷ is D; and in SEQ ID NO: 46, X⁸ is S. In some embodiments, in SEQ ID NO: 44, X¹ is A, X² is E, X³ is Q, and X⁴ is G; in SEQ ID NO: 45, X⁵ is K, X⁶ is V, and X⁷ is D; and in SEQ ID NO: 46, X⁸ is T.

In some embodiments, in SEQ ID NO: 44, X¹ is A, X² is E, X³ is Q, and X⁴ is G; in SEQ ID NO: 45, X⁵ is R, X⁶ is L, and X⁷ is N; and in SEQ ID NO: 46, X⁸ is S. In some embodiments, in SEQ ID NO: 44, X¹ is A, X² is E, X³ is 4, and X⁴ is G; in SEQ ID NO: 45, X⁵ is R, X⁶ is L, and X⁷ is N; and in SEQ ID NO: 46, X⁸ is T. In some embodiments, in SEQ ID NO: 44, X¹ is N, X² is E, X³ is Q and X⁴ is G; in SEQ ID NO: 45, X⁵ is R, X⁶ is V, and X⁷ is D; and in SEQ ID NO: 46, X⁸ is S. In some embodiments, in SEQ ID NO: 44, X¹ is N, X² is E, X³ is Q, and X⁴ is G; in SEQ ID NO: 45, X⁵ is R, X⁶ is V, and X⁷ is D; and in SEQ ID NO: 46, X⁸ is T. In some embodiments, in SEQ ID NO: 44, X¹ is N, X² is E, X³ is Q, and X⁴ is G; in SEQ ID NO: 45, X⁵ is K, X⁶ is V, and X⁷ is D; and in SEQ ID NO: 46, X⁸ is S. In some embodiments, in SEQ ID NO: 44, X¹ is N, X² is E, X³ is Q, and X⁴ is G; in SEQ ID NO: 45, X⁵ is K, X⁶ is V, and X⁷ is D; and in SEQ ID NO: 46, X⁸ is T. In some embodiments, in SEQ ID NO: 44, X¹ is N, X² is E, X³ is Q, and X⁴ is G; in SEQ ID NO: 45, X⁵ is R, X⁶ is L, and X⁷ is N; and in SEQ ID NO: 46, X⁸ is S. In some embodiments, in SEQ ID NO: 44, X¹ is N, X² is E, X³ is Q, and X⁴ is G; in SEQ ID NO: 45, X⁵ is R, X⁶ is L, and X⁷ is N; and in SEQ ID NO: 46, X⁸ is T. In some embodiments, in SEQ ID NO: 44, X¹ is N, X² is Q, X³ is Q, and X⁴ is G; in SEQ ID NO: 45, X⁵ is R, X⁶ is V, and X⁷ is D; and in SEQ ID NO: 46, X⁸ is S. In some embodiments, in SEQ ID NO: 44, X¹ is N, X² is Q, X³ is Q and X⁴ is G; in SEQ ID NO: 45, X⁵ is R, X⁶ is V, and X⁷ is D; and in SEQ ID NO: 46, X⁸ is T. In some embodiments, in SEQ ID NO: 44, X¹ is N, X² is Q, X³ is Q, and X⁴ is G; in SEQ ID NO: 45, X⁵ is K, X⁶ is V, and X⁷ is D; and in SEQ ID NO: 46, X⁸ is S. In some embodiments, in SEQ ID NO: 44, X¹ is N, X² is 4, X³ is Q and X⁴ is G; in SEQ ID NO: 45, X⁵ is K, X⁶ is V, and X⁷ is D; and in SEQ ID NO: 46, X⁸ is T. In some embodiments, in SEQ ID NO: 44, X¹ is N, X² is Q, X³ is Q, and X⁴ is G; in SEQ ID NO: 45, X⁵ is R, X⁶ is L, and X⁷ is N; and in SEQ ID NO: 46, X⁸ is S. In some embodiments, in SEQ ID NO: 44, X¹ is N, X² is Q, X³ is Q and X⁴ is G; in SEQ ID NO: 45, X⁵ is R, X⁶ is L, and X⁷ is N; and in SEQ ID NO: 46, X⁸ is T. In some embodiments, in SEQ ID NO: 44, X¹ is N, X² is Q, X³ is E, and X⁴ is G; in SEQ ID NO: 45, X⁵ is R, X⁶ is V, and X⁷ is D; and in SEQ ID NO: 46, X⁸ is S. In some embodiments, in SEQ ID NO: 44, X¹ is N, X² is Q, X³ is E, and X⁴ is G; in SEQ ID NO: 45, X⁵ is R, X⁶ is V, and X⁷ is D; and in SEQ ID NO: 46, X⁸ is T. In some embodiments, in SEQ ID NO: 44, X¹ is N, X² is Q, X³ is E, and X⁴ is G; in SEQ ID NO: 45, X⁵ is K, X⁶ is V, and X⁷ is D; and in SEQ ID NO: 46, X⁸ is S. In some embodiments, in SEQ ID NO: 44, X¹ is N, X² is Q, X³ is E, and X⁴ is G; in SEQ ID NO: 45, X⁵ is K, X⁶ is V, and X⁷ is D; and in SEQ ID NO: 46, X⁸ is T. In some embodiments, in SEQ ID NO: 44, X¹ is N, X² is Q, X³ is E, and X⁴ is G; in SEQ ID NO: 45, X⁵ is R, X⁶ is L, and X⁷ is N; and in SEQ ID NO: 46, X⁸ is S. In some embodiments, in SEQ ID NO: 44, X¹ is N, X² is Q, X³ is E, and X⁴ is G; in SEQ ID NO: 45, X⁵ is R, X⁶ is L, and X⁷ is N; and in SEQ ID NO: 46, X⁸ is T. In some embodiments, in SEQ ID NO: 44, X¹ is N, X² is Q, X³ is E, and X⁴ is D; in SEQ ID NO: 45, X⁵ is R, X⁶ is V, and X⁷ is D; and in SEQ ID NO: 46, X⁸ is S. In some embodiments, in SEQ ID NO: 44, X¹ is N, X² is Q, X³ is E, and X⁴ is D; in SEQ ID NO: 45, X⁵ is R, X⁶ is V, and X⁷ is D; and in SEQ ID NO: 46, X⁸ is T. In some embodiments, in SEQ ID NO: 44, X¹ is N, X² is Q, X³ is E, and X⁴ is D; in SEQ ID NO: 45, X⁵ is K, X⁶ is V, and X⁷ is D; and in SEQ ID NO: 46, X⁸ is S. In some embodiments, in SEQ ID NO: 44, X¹ is N, X² is Q, X³ is E, and X⁴ is D; in SEQ ID NO: 45, X⁵ is R, X⁶ is L, and X⁷ is N; and in SEQ ID NO: 46, X⁸ is S. In some embodiments, in SEQ ID NO: 44, X¹ is N, X² is Q, X³ is E, and X⁴ is D; in SEQ ID NO: 45, X⁵ is R, X⁶ is L, and X⁷ is N; and in SEQ ID NO: 46, X⁸ is T.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 26, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 26, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 26, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 35, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 26, heavy chain CDR3 (HCDR3) comprising SEQ ID NO: 27; light chain CDR1 (LCDR1) comprising SEQ ID NO: 34, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 26, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 23, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 24, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 21, heavy chain CDR2 (HCDR2) comprising SEQ ID NO: 25, heavy chain CDR3 (HCDR3) comprising SEQ ID NO: 27; light chain CDR1 (LCDR1) comprising SEQ ID NO: 33, light chain CDR2 (LCDR2) comprising SEQ ID NO: 36, and light chain CDR3 (LCDR3) comprising SEQ ID NO: 37, as defined by the Kabat numbering system.

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system (International ImMunoGeneTics Information System (IMGT®)).

In some embodiments, an anti-PSMA antibody or antigen-binding fragment thereof provided herein comprises three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 31; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system.

In some embodiments, an anti-PSMA antibody or antigen-binding fragment thereof provided herein comprises a heavy chain variable region of SEQ ID NO: 42, wherein SEQ ID NO: 42 comprises the amino acid sequence:

EVQLVQSGAEVKKPGATVKISCKX⁹SGYTFTEYTIHWVQQAPGKGLEWX¹⁰GNINPNNGGTTYX¹¹X¹²KFX¹³
X¹⁴RVTITX¹⁵DX¹⁶STX¹⁷TAYMELSSLRSEDTAVYYCAX¹⁸GWNFDYWGQGTLLTVSS

wherein X⁹ is V or T, X¹⁰ is M or I, X¹¹ is A or N, X¹² is E or Q, X¹³ is Q or E, X¹⁴ is G or D, X¹⁵ is A or V, X¹⁶ is T or K, X¹⁷ is D or S, and X¹⁸ is T or A. In some embodiments, an anti-PSMA antibody or antigen-binding fragment thereof provided herein comprises a light chain variable region of SEQ ID NO: 43, wherein SEQ ID NO: 43 comprises the amino acid sequence:

DIQMTQSPSSLSASVGDRVTITCX¹⁹ASQDVGTAX²⁰x²¹WYQQKPGKAPKLLIYWASTRHX²²GVPSRFSGS
GSGTDFTLTISSLQPEDFATYYCQQYNSYPLTFGQGTKLEIK

wherein X¹⁹ is R or K, X²⁰ is L or V, X²¹ is N or D, and X²² is S or T. In some embodiments, the antibody or antigen-binding fragment comprises a heavy chain variable region of SEQ ID NO: 42 and a light chain variable region of SEQ ID NO: 43, wherein X⁹ is V or T, X¹⁰ is M or I, X¹¹ is A or N, X¹² is E or Q, X¹³ is Q or E, X¹⁴ is G or D, X¹⁵ is A or V, X¹⁶ is T or K, X¹⁷ is D or S, X¹⁸ is T or A, X¹⁹ is R or K, X²⁰ is L or V, X²¹ is N or D, and X²² is S or T.

In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and/or X¹⁸ is T. In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and/or X¹⁸ is T. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and/or X¹⁸ is T. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and/or X¹⁸ is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and/or X¹⁸ is A. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and/or X¹⁸ is A. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and/or X¹⁸ is A. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and/or X¹⁸ is A. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and/or X¹⁸ is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and/or X¹⁸ is T. In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X¹⁷ is D, and/or X¹⁸ is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is T, X¹⁷ is D, and/or X¹⁸ is T. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and/or X¹⁸ is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and/or X¹⁸ is T. In some embodiments, in SEQ ID NO: 43, X¹⁹ is K, X²⁰ is V, X²¹ is D, and/or X²² is T. In some embodiments, in SEQ ID NO: 43, X¹⁹ is R, X²⁰ is V, X²¹ is D, and/or X²² is T. In some embodiments, in SEQ ID NO: 43, X¹⁹ is R, X²⁰ is L, X²¹ is N, and/or X²² is T. In some embodiments, in SEQ ID NO: 43, X¹⁹ is K, X²⁰ is V, X²¹ is D, and/or X²² is S. In some embodiments, in SEQ ID NO: 43, X¹⁹ is R, X²⁰ is V, X²¹ is D, and/or X²² is S. In some embodiments, in SEQ ID NO: 43, X¹⁹ is R, X²⁰ is L, X²¹ is N, and/or X²² is S.

In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42, X⁹ is V, X¹⁰ is M, X¹¹ is A, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is T.

In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is E, X³ is Q, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42, X⁹ is V, X¹⁰ is M, X¹¹ is N, X¹² is E, X¹³ is Q, X¹⁴ is G, X¹⁵ is A, X¹⁶ is T, X¹⁷ is D, and X¹⁸ is T, and in SEQ ID NO: 43, X⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is 4, X¹³ is 4, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42, X⁹ is V, X¹⁰ is M, X¹¹ is N, X¹² is Q, X¹³ is Q, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is T.

In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is G, X¹⁵ is A, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and X¹⁸ is A, and in SEQ ID NO: 43, X¹⁹ is K, X²⁰ is V, X²¹ is D, and X²² is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and X¹⁸ is A, and in SEQ ID NO: 43, X¹⁹ is R, X²⁰ is V, X²¹ is D, and X²² is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and X¹⁸ is A, and in SEQ ID NO: 43, X¹⁹ is R, X²⁰ is L, X²¹ is N, and X²² is T. In some embodiments, in SEQ ID NO: 42, X⁹ is T, X¹⁰ is I, X¹¹ is N, X¹² is 4, X¹³ is E, X¹⁴ is D, X¹⁵ is A, X¹⁶ is T, X¹⁷ is D, and X¹⁸ is A, and in SEQ ID NO: 43, X¹⁹ is K, X²⁰ is V, X²¹ is D, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and X¹⁸ is A, and in SEQ ID NO: 43, X¹⁹ is R, X²⁰ is V, X²¹ is D, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is T, X¹⁷ is D, and X¹⁸ is A, and in SEQ ID NO: 43, X¹⁹ is R, X²⁰ is L, X²¹ is N, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is K, X²⁰ is V, X²¹ is D, and X²² is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is R, X²⁰ is V, X²¹ is D, and X²² is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is R, X²⁰ is L, X²¹ is N, and X²² is T. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is K, X²⁰ is V, X²¹ is D, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is R, X²⁰ is V, X²¹ is D, and X²² is S. In some embodiments, in SEQ ID NO: 42, X⁹ is T, X¹⁰ is I, X¹¹ is N, X¹² is Q, X¹³ is E, X¹⁴ is D, X¹⁵ is A, X¹⁶ is K, X¹⁷ is S, and X¹⁸ is A, and in SEQ ID NO: 43, X¹⁹ is R, X²⁰ is L, X²¹ is N, and X²² is S. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is K, X²⁰ is V, X²¹ is D, and X²² is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is V, X²¹ is D, and X²² is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is L, X²¹ is N, and X²² is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is K, X²⁰ is V, X²¹ is D, and X²² is S. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is V, X²¹ is D, and X²² is S. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is V, X²¹ is D, and X²² is S. In some embodiments, in SEQ ID NO: 42 X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is A, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is K, X²⁰ is V, X²¹ is D, and X²² is T.

In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is R, X²⁰ is V, X²¹ is D, and X²² is S. In some embodiments, in SEQ ID NO: 42 X⁹ is V, X¹⁰ is M, X¹¹ is N, X¹² is Q, X¹³ is E, X¹⁴ is D, X¹⁵ is A, X¹⁶ is T, X¹⁷ is S, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is R, X²⁰ is V, X²¹ is D, and X²² is T.

In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is R, X²⁰ is V, X²¹ is D, and X²² is S. In some embodiments, in SEQ ID NO: 42 X⁹ is V, X¹⁰ is M, X¹¹ is N, X¹² is Q, X¹³ is E, X¹⁴ is D, X¹⁵ is A, X¹⁶ is K, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X¹⁷ is D, and X¹⁸ is T, and in SEQ ID NO: 43, X¹⁹ is R, X²⁰ is L, X²¹ is N, and X²² is T.

In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is D, X¹⁵ is V, X¹⁶ is T, X⁷ is D, and X¹⁸ is T, and in SEQ ID NO: 43, X¹⁹ is R, X²⁰ is V, X²¹ is D, and X²² is S. In some embodiments, in SEQ ID NO: 42 X⁹ is V, X¹⁰ is M, X¹¹ is N, X¹² is Q, X¹³ is E, X¹⁴ is D, X¹⁵ is V, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is I, X¹¹ is N, X¹² is 4, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is I, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42 X⁹ is V, X¹⁰ is I, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is S.

In some embodiments, in SEQ ID NO: 42 X⁹ is T, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is T. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is K, X²⁰ is V, X²¹ is D, and X²² is S. In some embodiments, in SEQ ID NO: 42, X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is V, X²¹ is D, and X²² is S. In some embodiments, in SEQ ID NO: 42 X⁹ is V, X¹⁰ is M, X¹¹ is N, X¹² is Q, X¹³ is E, X¹⁴ is D, X¹⁵ is A, X¹⁶ is T, X¹⁷ is D, and X¹⁸ is T, and in SEQ ID NO: 43, X¹⁹ is R, X²⁰ is L, X²¹ is N, and X²² is S.

In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 1, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 2, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 3, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 4, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 5, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 6, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 7, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 8, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 9, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 10, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 11, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 12, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 13, and a light chain variable region comprising the amino acid sequence of SEQ ID NO: 20. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 15. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 16. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 17. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 18. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 19. In some embodiments, an anti-PSMA antibody or antigen-binding fragment thereof provided herein comprises a heavy chain variable region comprising the amino acid sequence of SEQ ID NO: 14, and a light chain variable region comprising the amino acid sequence of SEQ ID NO: 20.

In some embodiments, the anti-PSMA antibody or antigen-binding fragment thereof comprises a heavy chain variable region comprising the amino acid sequence of SEQ ID NO: 7, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 8, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 9, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 10, and a light chain variable region comprising the amino acid sequence of SEQ ID NO: 15.

In some embodiments, the anti-PSMA antibody or antigen-binding fragment thereof comprises a heavy chain variable region comprising the amino acid sequence of SEQ ID NO: 1, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 2, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 3, and a light chain variable region comprising the amino acid sequence of SEQ ID NO: 15. In some embodiments, the anti-PSMA antibody or antigen-binding fragment thereof comprises a heavy chain variable region comprising the amino acid sequence of SEQ ID NO: 14, and a light chain variable region comprising the amino acid sequence of SEQ ID NO: 15.

In some embodiments, the anti-PSMA antibody or antigen-binding fragment thereof comprises a heavy chain variable region comprising the amino acid sequence of SEQ ID NO: 14, and a light chain variable region comprising the amino acid sequence of SEQ ID NO: 19.

In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2. In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3. In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 14.

In some embodiments, an anti-PSMA antibody or antigen-binding fragment thereof provided herein comprises a light chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 15. In some embodiments, an anti-PSMA antibody or antigen-binding fragment thereof provided herein comprises a light chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 19.

In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 40 (i.e., does not share 100% identity with SEQ ID NO: 40), wherein the anti-PSMA antibody or antigen binding fragment provides improved properties over other anti-PSMA antibodies, e.g., J591 and/or deJ591. In some embodiments, an anti-PSMA antibody or antigen-binding fragment thereof provided herein comprises a light chain variable region with at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 41 (i.e., does not share 100% identity with SEQ ID NO: 41), wherein the anti-PSMA antibody or antigen binding fragment provides improved properties over other anti-PSMA antibodies, e.g., J591 and/or deJ591. In some embodiments, the anti-PSMA antibody or antigen-binding fragment comprises a heavy chain variable region with at least 86% identity to SEQ ID NO: 40 and a light chain variable region with at least 87% identity to SEQ ID NO: 41 (but not 100% identity to either variable region), wherein the anti-PSMA antibody or antigen-binding fragment provides improved properties over other anti-PSMA antibodies, e.g., J591 and/or deJ591. The improved properties may include superior stability and/or less immunogenicity.

In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 40, wherein the anti-PSMA antibody or antigen-binding fragment comprises at least the following amino acids that differ from SEQ ID NO: 40:

| Position relative to SEQ ID NO: 40 | Amino Acid |
|---|---|
| 9 | A |
| 38 | Q |
| 67 | R |
| 68 | V |
| 70 | I |
| 111 | V |

wherein the anti-PSMA antibody or antigen binding fragment provides improved properties over other anti-PSMA antibodies, e.g., J591 and/or deJ591. The improved properties may include superior stability and/or less immunogenicity. The position relative to SEQ ID NO: 40 is determined by aligning the heavy chain variable region of the anti-PSMA antibody or antigen-binding fragment with SEQ ID NO: 40, optionally using the BLAST algorithm, then counting the amino acid position starting from the N terminal of the aligned sequences.

In some embodiments, an anti-PSMA antibody or antigen-binding fragment thereof provided herein comprises a light chain variable region with at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 41, wherein the anti-PSMA antibody or antigen-binding fragment comprises at least the following amino acids that differ from SEQ ID NO: 41:

| Position relative to SEQ ID NO: 41 | Amino Acid |
|---|---|
| 13 | A |
| 21 | I |
| 42 | K |
| 43 | A |
| 58 | V |
| 85 | T |
| 100 | Q |
| 104 | L |
| 105 | E |

wherein the anti-PSMA antibody or antigen binding fragment provides improved properties over other anti-PSMA antibodies, e.g., J591 and/or deJ591. The improved properties may include superior stability and/or less immunogenicity. The position relative to SEQ ID NO: 41 is determined by aligning the heavy chain variable region of the anti-PSMA antibody or antigen-binding fragment with SEQ ID NO: 41, optionally using the BLAST algorithm, then counting the amino acid position starting from the N terminal of the aligned sequences.

In some embodiments, an anti-PSMA antibody or antigen-binding fragment thereof provided herein comprises a heavy chain variable region with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 42, wherein the anti-PSMA antibody or antigen binding fragment provides improved properties over other anti-PSMA antibodies, e.g., J591 and/or deJ591. In some embodiments, an anti-PSMA antibody or antigen-binding fragment thereof provided herein comprises a light chain variable region with at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 43, wherein the anti-PSMA antibody or antigen binding fragment provides improved properties over other anti-PSMA antibodies, e.g., J591 and/or deJ591. The anti-PSMA antibody or antigen-binding fragment does not comprise a heavy chain variable region with 100% identity to SEQ ID NO: 40 and a light chain variable region with 100% identity to SEQ ID NO: 41. The improved properties may include superior stability and/or less immunogenicity.

In various embodiments, any of the anti-PSMA antibodies disclosed herein may comprise a human IgG1 Fc domain. In some embodiments, an anti-PSMA antibody comprises a human IgG1 Fc domain that is modified to reduce binding to an FcγR as compared to an IgG1 Fc-containing antibody with a wild type IgG1 Fc domain. In some embodiments, the anti-PSMA antibodies comprise a mutated human IgG1 Fc domain that comprises one or more of (e.g., all of) L234A, L235A, P238S, H268Q, and K274Q modifications to a human IgG1 heavy chain constant domain.

In various embodiments, the anti-PSMA antibodies comprise a human Ig kappa light chain constant region. In various embodiments, the anti-PSMA antibodies comprise a human Ig lambda light chain constant region.

In some embodiments, an anti-PSMA antibody provided herein comprises a heavy chain comprising an amino acid sequence selected from SEQ ID NOs: 47-60, and a light chain comprising an amino acid sequence selected from SEQ ID NOs: 61-66.

In some embodiments, the anti-PSMA antibody comprises the heavy chain amino acid sequence of SEQ ID NO: 47 and the light chain amino acid sequence of SEQ ID NO: 61. In some embodiments, the anti-PSMA antibody comprises the heavy chain amino acid sequence of SEQ ID NO: 47 and the light chain amino acid sequence of SEQ ID NO: 62. In some embodiments, the anti-PSMA antibody comprises the heavy chain amino acid sequence of SEQ ID NO: 47 and the light chain amino acid sequence of SEQ ID NO: 63. In some embodiments, the anti-PSMA antibody comprises the heavy chain amino acid sequence of SEQ ID NO: 47 and the light chain amino acid sequence of SEQ ID NO: 64. In some embodiments, the anti-PSMA antibody comprises the heavy chain amino acid sequence of SEQ ID NO: 47 and the light chain amino acid sequence of SEQ ID NO: 65. In some embodiments, the anti-PSMA antibody comprises the heavy chain amino acid sequence of SEQ ID NO: 47 and the light chain amino acid sequence of SEQ ID NO: 66. In some embodiments, the anti-PSMA antibody comprises the heavy chain amino acid sequence of SEQ ID NO: 48 and the light chain amino acid sequence of SEQ ID NO: 61. In some embodiments, the anti-PSMA antibody comprises the heavy chain amino acid sequence of SEQ ID NO: 48 and the light chain amino acid sequence of SEQ ID NO: 62. In some embodiments, the anti-PSMA antibody comprises the heavy chain amino acid sequence of SEQ ID NO: 48 and the light chain amino acid sequence of SEQ ID NO: 63. In some embodiments, the anti-PSMA antibody comprises the heavy chain amino acid sequence of SEQ ID NO: 48 and the light chain amino acid sequence of SEQ ID NO: 64. In some embodiments, the anti-PSMA antibody comprises the heavy chain amino acid sequence of SEQ ID NO: 48 and the light chain amino acid sequence of SEQ ID NO: 65. In some embodiments, the anti-PSMA antibody comprises the heavy chain amino acid sequence of SEQ ID NO: 48 and the light chain amino acid sequence of SEQ ID NO: 66. In some embodiments, the anti-PSMA antibody comprises the heavy chain amino acid sequence of SEQ ID NO: 49 and the light chain amino acid sequence of SEQ ID NO: 61. In some embodiments, the anti-PSMA antibody comprises the heavy chain amino acid sequence of SEQ ID NO: 49 and the light chain amino acid sequence of SEQ ID NO: 62. In some embodiments, the anti-PSMA antibody comprises the heavy chain amino acid sequence of SEQ ID NO: 49 and the light chain amino acid sequence of SEQ ID NO: 63. In some embodiments, the anti-PSMA antibody comprises the heavy chain amino acid sequence of SEQ ID NO: 49 and the light chain amino acid sequence of SEQ ID NO: 64. In some embodiments, the anti-PSMA antibody comprises the heavy chain amino acid sequence of SEQ ID NO: 49 and the light chain amino acid sequence of SEQ ID NO: 65. In some embodiments, the anti-PSMA antibody comprises the heavy chain amino acid sequence of SEQ ID NO: 49 and the light chain amino acid sequence of SEQ ID NO: 66. In some embodiments, the anti-PSMA antibody comprises the heavy chain amino acid sequence of SEQ ID NO: 50 and the light chain amino acid sequence of SEQ ID NO: 61. In some embodiments, the anti-PSMA antibody comprises the heavy chain amino acid sequence of SEQ ID NO: 50 and the light chain amino acid sequence of SEQ ID NO: 62. In some embodiments, the anti-PSMA antibody comprises the heavy chain amino acid sequence of SEQ ID NO: 50 and the light chain amino acid sequence of SEQ ID NO: 63. In some embodiments, the anti-PSMA antibody comprises the heavy chain amino acid sequence of SEQ ID NO: 50 and the light chain amino acid sequence of SEQ ID NO: 64. In some embodiments, the anti-PSMA antibody comprises the heavy chain amino acid sequence of SEQ ID NO: 50 and the light chain amino acid sequence of SEQ ID NO: 65. In some embodiments, the anti-PSMA antibody comprises the heavy chain amino acid sequence of SEQ ID NO: 50 and the light chain amino acid sequence of SEQ ID NO: 66. In some embodiments, the anti-PSMA antibody comprises the heavy chain amino acid sequence of SEQ ID NO: 51 and the light chain amino acid sequence of SEQ ID NO: 61. In some embodiments, the anti-PSMA antibody comprises the heavy chain amino acid sequence of SEQ ID NO: 51 and the light chain amino acid sequence of SEQ ID NO: 62. In some embodiments, the anti-PSMA antibody comprises the heavy chain amino acid sequence of SEQ ID NO: 51 and the light chain amino acid sequence of SEQ ID NO: 63. In some embodiments, the anti-PSMA antibody comprises the heavy chain amino acid sequence of SEQ ID NO: 51 and the light chain amino acid sequence of SEQ ID NO: 64. In some embodiments, the anti-PSMA antibody comprises the heavy chain amino acid sequence of SEQ ID NO: 51 and the light chain amino acid sequence of SEQ ID NO: 65. In some embodiments, the anti-PSMA antibody comprises the heavy chain amino acid sequence of SEQ ID NO: 51 and the light chain amino acid sequence of SEQ ID NO: 66. In some embodiments, the anti-PSMA antibody comprises the heavy chain amino acid sequence of SEQ ID NO: 52 and the light chain amino acid sequence of SEQ ID NO: 61. In some embodiments, the anti-PSMA antibody comprises the heavy chain amino acid sequence of SEQ ID NO: 52 and the light chain amino acid sequence of SEQ ID NO: 62. In some embodiments, the anti-PSMA antibody comprises the heavy chain amino acid sequence of SEQ ID NO: 52 and the light chain amino acid sequence of SEQ ID NO: 63. In some embodiments, the anti-PSMA antibody comprises the heavy chain amino acid sequence of SEQ ID NO: 52 and the light chain amino acid sequence of SEQ ID NO: 64. In some embodiments, the anti-PSMA antibody comprises the heavy chain amino acid sequence of SEQ ID NO: 52 and the light chain amino acid sequence of SEQ ID NO: 65. In some embodiments, the anti-PSMA antibody comprises the heavy chain amino acid sequence of SEQ ID NO: 52 and the light chain amino acid sequence of SEQ ID NO: 66. In some embodiments, the anti-PSMA antibody comprises the heavy chain amino acid sequence of SEQ ID NO: 53 and the light chain amino acid sequence of SEQ ID NO: 61. In some embodiments, the anti-PSMA antibody comprises the heavy chain amino acid sequence of SEQ ID NO: 53 and the light chain amino acid sequence of SEQ ID NO: 62. In some embodiments, the anti-PSMA antibody comprises the heavy chain amino acid sequence of SEQ ID NO: 53 and the light chain amino acid sequence of SEQ ID NO: 63. In some embodiments, the anti-PSMA antibody comprises the heavy chain amino acid sequence of SEQ ID NO: 53 and the light chain amino acid sequence of SEQ ID NO: 64. In some embodiments, the anti-PSMA antibody comprises the heavy chain amino acid sequence of SEQ ID NO: 53 and the light chain amino acid sequence of SEQ ID NO: 65. In some embodiments, the anti-PSMA antibody comprises the heavy chain amino acid sequence of SEQ ID NO: 53 and the light chain amino acid sequence of SEQ ID NO: 66. In some embodiments, the anti-PSMA antibody comprises the heavy chain amino acid sequence of SEQ ID NO: 54 and the light chain amino acid sequence of SEQ ID NO: 61. In some embodiments, the anti-PSMA antibody comprises the heavy chain amino acid sequence of SEQ ID NO: 54 and the light chain amino acid sequence of SEQ ID NO: 62. In some embodiments, the anti-PSMA antibody comprises the heavy chain amino acid sequence of SEQ ID NO: 54 and the light chain amino acid sequence of SEQ ID NO: 63. In some embodiments, the anti-PSMA antibody comprises the heavy chain amino acid sequence of SEQ ID NO: 54 and the light chain amino acid sequence of SEQ ID NO: 64. In some embodiments, the anti-PSMA antibody comprises the heavy chain amino acid sequence of SEQ ID NO: 54 and the light chain amino acid sequence of SEQ ID NO: 65. In some embodiments, the anti-PSMA antibody comprises the heavy chain amino acid sequence of SEQ ID NO: 54 and the light chain amino acid sequence of SEQ ID NO: 66. In some embodiments, the anti-PSMA antibody comprises the heavy chain amino acid sequence of SEQ ID NO: 55 and the light chain amino acid sequence of SEQ ID NO: 61. In some embodiments, the anti-PSMA antibody comprises the heavy chain amino acid sequence of SEQ ID NO: 55 and the light chain amino acid sequence of SEQ ID NO: 62. In some embodiments, the anti-PSMA antibody comprises the heavy chain amino acid sequence of SEQ ID NO: 55 and the light chain amino acid sequence of SEQ ID NO: 63. In some embodiments, the anti-PSMA antibody comprises the heavy chain amino acid sequence of SEQ ID NO: 55 and the light chain amino acid sequence of SEQ ID NO: 64. In some embodiments, the anti-PSMA antibody comprises the heavy chain amino acid sequence of SEQ ID NO: 55 and the light chain amino acid sequence of SEQ ID NO: 65. In some embodiments, the anti-PSMA antibody comprises the heavy chain amino acid sequence of SEQ ID NO: 55 and the light chain amino acid sequence of SEQ ID NO: 66. In some embodiments, the anti-PSMA antibody comprises the heavy chain amino acid sequence of SEQ ID NO: 56 and the light chain amino acid sequence of SEQ ID NO: 61. In some embodiments, the anti-PSMA antibody comprises the heavy chain amino acid sequence of SEQ ID NO: 56 and the light chain amino acid sequence of SEQ ID NO: 62. In some embodiments, the anti-PSMA antibody comprises the heavy chain amino acid sequence of SEQ ID NO: 56 and the light chain amino acid sequence of SEQ ID NO: 63. In some embodiments, the anti-PSMA antibody comprises the heavy chain amino acid sequence of SEQ ID NO: 56 and the light chain amino acid sequence of SEQ ID NO: 64. In some embodiments, the anti-PSMA antibody comprises the heavy chain amino acid sequence of SEQ ID NO: 56 and the light chain amino acid sequence of SEQ ID NO: 65. In some embodiments, the anti-PSMA antibody comprises the heavy chain amino acid sequence of SEQ ID NO: 56 and the light chain amino acid sequence of SEQ ID NO: 66. In some embodiments, the anti-PSMA antibody comprises the heavy chain amino acid sequence of SEQ ID NO: 57 and the light chain amino acid sequence of SEQ ID NO: 61. In some embodiments, the anti-PSMA antibody comprises the heavy chain amino acid sequence of SEQ ID NO: 57 and the light chain amino acid sequence of SEQ ID NO: 62. In some embodiments, the anti-PSMA antibody comprises the heavy chain amino acid sequence of SEQ ID NO: 57 and the light chain amino acid sequence of SEQ ID NO: 63. In some embodiments, the anti-PSMA antibody comprises the heavy chain amino acid sequence of SEQ ID NO: 57 and the light chain amino acid sequence of SEQ ID NO: 64. In some embodiments, the anti-PSMA antibody comprises the heavy chain amino acid sequence of SEQ ID NO: 57 and the light chain amino acid sequence of SEQ ID NO: 65. In some embodiments, the anti-PSMA antibody comprises the heavy chain amino acid sequence of SEQ ID NO: 57 and the light chain amino acid sequence of SEQ ID NO: 66. In some embodiments, the anti-PSMA antibody comprises the heavy chain amino acid sequence of SEQ ID NO: 58 and the light chain amino acid sequence of SEQ ID NO: 61. In some embodiments, the anti-PSMA antibody comprises the heavy chain amino acid sequence of SEQ ID NO: 58 and the light chain amino acid sequence of SEQ ID NO: 62. In some embodiments, the anti-PSMA antibody comprises the heavy chain amino acid sequence of SEQ ID NO: 58 and the light chain amino acid sequence of SEQ ID NO: 63. In some embodiments, the anti-PSMA antibody comprises the heavy chain amino acid sequence of SEQ ID NO: 58 and the light chain amino acid sequence of SEQ ID NO: 64. In some embodiments, the anti-PSMA antibody comprises the heavy chain amino acid sequence of SEQ ID NO: 58 and the light chain amino acid sequence of SEQ ID NO: 65. In some embodiments, the anti-PSMA antibody comprises the heavy chain amino acid sequence of SEQ ID NO: 58 and the light chain amino acid sequence of SEQ ID NO: 66. In some embodiments, the anti-PSMA antibody comprises the heavy chain amino acid sequence of SEQ ID NO: 59 and the light chain amino acid sequence of SEQ ID NO: 61. In some embodiments, the anti-PSMA antibody comprises the heavy chain amino acid sequence of SEQ ID NO: 59 and the light chain amino acid sequence of SEQ ID NO: 62. In some embodiments, the anti-PSMA antibody comprises the heavy chain amino acid sequence of SEQ ID NO: 59 and the light chain amino acid sequence of SEQ ID NO: 63. In some embodiments, the anti-PSMA antibody comprises the heavy chain amino acid sequence of SEQ ID NO: 59 and the light chain amino acid sequence of SEQ ID NO: 64. In some embodiments, the anti-PSMA antibody comprises the heavy chain amino acid sequence of SEQ ID NO: 59 and the light chain amino acid sequence of SEQ ID NO: 65. In some embodiments, the anti-PSMA antibody comprises the heavy chain amino acid sequence of SEQ ID NO: 59 and the light chain amino acid sequence of SEQ ID NO: 66. In some embodiments, the anti-PSMA antibody comprises the heavy chain amino acid sequence of SEQ ID NO: 60 and the light chain amino acid sequence of SEQ ID NO: 61. In some embodiments, the anti-PSMA antibody comprises the heavy chain amino acid sequence of SEQ ID NO: 60 and the light chain amino acid sequence of SEQ ID NO: 62. In some embodiments, the anti-PSMA antibody comprises the heavy chain amino acid sequence of SEQ ID NO: 60 and the light chain amino acid sequence of SEQ ID NO: 63. In some embodiments, the anti-PSMA antibody comprises the heavy chain amino acid sequence of SEQ ID NO: 60 and the light chain amino acid sequence of SEQ ID NO: 64. In some embodiments, the anti-PSMA antibody comprises the heavy chain amino acid sequence of SEQ ID NO: 60 and the light chain amino acid sequence of SEQ ID NO: 65. In some embodiments, the anti-PSMA antibody comprises the heavy chain amino acid sequence of SEQ ID NO: 60 and the light chain amino acid sequence of SEQ ID NO: 66.

In some embodiments, the anti-PSMA antibody comprises the heavy chain amino acid sequence of SEQ ID NO: 47 and the light chain amino acid sequence of SEQ ID NO: 61. In some embodiments, the anti-PSMA antibody comprises the heavy chain amino acid sequence of SEQ ID NO: 48 and the light chain amino acid sequence of SEQ ID NO: 61. In some embodiments, the anti-PSMA antibody comprises the heavy chain amino acid sequence of SEQ ID NO: 49 and the light chain amino acid sequence of SEQ ID NO: 61. In some embodiments, the anti-PSMA antibody comprises the heavy chain amino acid sequence of SEQ ID NO: 60 and the light chain amino acid sequence of SEQ ID NO: 61.

In some embodiments, the anti-PSMA antibody comprises the heavy chain amino acid sequence of SEQ ID NO: 60 and the light chain amino acid sequence of SEQ ID NO: 65.

In any of the antibodies discussed above, the heavy chain amino acid sequence may lack the C-terminal lysine.

In various embodiments, amino acid substitutions may be made while retaining the binding affinity and/or specificity of an antibody disclosed herein and/or to provide one or more additional beneficial property, e.g., by making one or more changes in framework, constant domain, and/or CDR sequences. In some embodiments, the substitutions are of single residues. For instance, in some embodiments, the anti-PSMA antibodies comprise a human IgG1 Fc domain that comprises amino acid substitutions to reduce binding to an FcγR as compared to an IgG1 Fc-containing antibody with a wild type IgG1 Fc domain. In some embodiments, the anti-PSMA antibodies comprise a mutated human IgG1 Fc domain that comprises the substitutions L234A, L235A, P238S, H268Q, and K274Q. Insertions usually will be on the order of from about 1 to about 20 amino acid residues, although considerably larger insertions may be tolerated as long as biological function is retained (e.g., binding to PSMA). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions are generally made in accordance with the following chart depicted in Table 11.

TABLE 11

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In various embodiments where variant antibody sequences are used in an ADC, the variants may exhibit the same qualitative biological activity and will elicit the same immune response, although variants may also be selected to modify the characteristics of the antigen binding proteins as needed. For example, the anti-PSMA antibodies provided herein may comprise a human IgG1 Fc domain that is mutated to reduce binding to an FcγR as compared to an IgG1 Fc-containing antibody with a wild type IgG1 Fc domain. Alternatively, the variant may be designed such that the biological activity of the antigen binding protein is altered.

Any of the anti-PSMA antibodies and antigen binding fragments disclosed herein may be used as a conjugate, e.g., with a detectable agent and/or another therapeutic agent. In some embodiments the anti-PSMA antibody or antigen binding fragment may be used in an antibody-drug conjugate (ADC), e.g., any of the ADCs disclosed herein, preferably to target the drug in the ADC to a cancer cell. As shown below, the linker-toxins in the ADCs disclosed herein are surprisingly effective with the anti-PSMA antibodies also disclosed herein. These antibodies may be used with the linkers and toxin (e.g., Compound 1) disclosed herein.

### Linkers

In various embodiments, the anti-PSMA antibodies and antigen-binding fragments disclosed herein may be joined to a drug moiety (e.g., a cytotoxic payload, e.g., Compound 1) by a linker to create an antibody-drug conjugate (ADC).

In some embodiments, the linker in an ADC is stable extracellularly in a sufficient manner to be therapeutically effective. In some embodiments, the linker is stable outside a cell, such that the ADC remains intact when present in extracellular conditions (e.g., prior to transport or delivery into a cell). The term “intact,” used in the context of an ADC, means that the antibody moiety remains attached to the drug moiety (e.g., Compound 1). As used herein, “stable,” in the context of a linker or ADC comprising a linker, means that no more than about 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions when evaluated over a set period of time. In some embodiments, the linkers in ADCs disclosed herein are chosen to remain stable for more than about 48 hours, more than about 60 hours, more than about 72 hours, more than about 84 hours, or more than about 96 hours.

Whether a linker is stable extracellularly can be determined, for example, by including an ADC in plasma for a predetermined time period (e.g., 2, 4, 6, 8, 16, or 24 hours) and then quantifying the amount of free drug moiety present in the plasma. Stability may allow the ADC time to localize to target tumor cells and prevent the premature release of the drug, which could lower the therapeutic index of the ADC by indiscriminately damaging both normal and tumor tissues. In some embodiments, the linker is stable outside of a target cell and releases the drug moiety from the ADC once inside of the cell, such that the drug moiety can bind to its target (e.g., to STING). Thus, an effective linker will: (i) maintain the specific binding properties of the antibody moiety; (ii) allow delivery, e.g., intracellular delivery, of the drug moiety via stable attachment to the antibody moiety; (iii) remain stable and intact until the ADC has been transported or delivered to its target site; and (iv) allow for the therapeutic effect, e.g., cytotoxic effect, of the drug moiety after cleavage.

Linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, linkers disclosed herein result in ADCs with low aggregation levels and desirable levels of drug loading. In various embodiments, a linker is conjugated to the antibody or antigen-binding fragment through a cysteine. In various embodiments, a linker is conjugated to the antibody or antigen-binding fragment through a lysine. Suitable methods for conjugating linkers of the present disclosure to an antibody include the technologies for directed attachment to a lysine on a heavy chain of an antibody, to a cysteine on the heavy chain of an antibody, and to a cysteine on the light chain of an antibody, e.g., as disclosed in PCT applications WO 2017/213267, WO 2017/106643, and WO 2016/205618, and in Junutula et al. (2008) Journal of Immunological Methods 332:41-52, all of which are herein incorporated by reference in their entireties. In some embodiments, a linker is conjugated to the antibody or antigen-binding fragment on the light chain, e.g., at a cysteine on the light chain, e.g., at cysteine-80 on the light chain. In some embodiments, a linker is conjugated to the antibody or antigen-binding fragment on the heavy chain, e.g., at a cysteine on the heavy chain, e.g., at cysteine-118 on the heavy chain.

A linker used herein may be “cleavable” or “non-cleavable” (Ducry and Stump, Bioconjugate Chem. (2010) 21:5-13). Cleavable linkers are designed to release the drug when subjected to certain environment factors, e.g., when internalized into the target cell, whereas non-cleavable linkers generally rely on the degradation of the antibody moiety itself.

In some embodiments, the linker is a non-cleavable linker. In some embodiments, the drug moiety of the ADC is released by degradation of the antibody moiety.

In some embodiments, the linker is cleavable. Cleavable linkers are designed to release the drug when subjected to certain environmental factors, e.g., when internalized into the target cell. A cleavable linker refers to any linker that comprises a cleavable moiety. As used herein, the term “cleavable moiety” refers to any chemical bond that can be cleaved. Suitable cleavable chemical bonds are known in the art and include, but are not limited to, acid labile bonds, protease/peptidase labile bonds, photolabile bonds, disulfide bonds, and esterase labile bonds. Linkers comprising a cleavable moiety can allow for the release of the drug moiety from the ADC via cleavage at a particular site in the linker.

In some embodiments, the linker is cleavable under intracellular conditions, such that cleavage of the linker sufficiently releases the drug moiety from the antibody moiety in the intracellular environment to activate the drug and/or render the drug therapeutically effective. In some embodiments, the drug moiety is not cleaved from the antibody moiety until the ADC enters a cell that expresses an antigen specific for the antibody moiety of the ADC, and the drug moiety is cleaved from the antibody moiety upon entering the cell. In some embodiments, the linker comprises a cleavable moiety that is positioned such that no part of the linker or the antibody moiety remains bound to the drug moiety upon cleavage. Exemplary cleavable linkers include acid labile linkers, protease/peptidase-sensitive linkers, photolabile linkers, dimethyl-, disulfide-, or sulfonamide-containing linkers.

In some embodiments, the linker is cleavable by a cleaving agent, e.g., an enzyme, that is present in the intracellular environment (e.g., within a lysosome, endosome, or caveolea). The linker can be, e.g., a peptide linker that is cleaved by an intracellular peptidase or protease enzyme, including, but not limited to, a lysosomal or endosomal protease. In some embodiments, the linker is a cleavable peptide linker. As used herein, a cleavable peptide linker refers to any linker that comprises a cleavable peptide moiety. The term “cleavable peptide moiety” refers to any chemical bond linking amino acids (natural or synthetic amino acid derivatives) that can be cleaved by an agent that is present in the intracellular environment. In some embodiments, a cleavable peptide linker is more stably conjugated to an antibody disclosed herein compared to an acid labile linker.

In some embodiments, the linker is an enzyme-cleavable linker and a cleavable peptide moiety in the linker is cleavable by the enzyme. In some embodiments, the cleavable peptide moiety is cleavable by a lysosomal enzyme, e.g., cathepsin or legumain (also known as asparaginyl endopeptidase or vacuolar processing enzyme). In some embodiments, the linker is a cathepsin-cleavable linker. In some embodiments, the linker is a legumain-cleavable linker. In some embodiments, the cleavable peptide moiety in the linker is cleavable by a lysosomal cysteine cathepsin, such as cathepsin B, C, F, H, K, L, O, S, V, X, or W. In some embodiments, the cleavable peptide moiety is cleavable by cathepsin B. An exemplary dipeptide that may be cleaved by cathepsin B is valine-citrulline (Val-Cit). See, e.g., Dubowchik et al. (2002) Bioconjugate Chem. 13:855-69. Another exemplary dipeptide that may be cleaved by cathepsin B is valine-alanine (Val-Ala). See, e.g., Fu and Ho (2002) Antib. Ther. 1(2):33-43.

In some embodiments, the cleavable peptide moiety in the linker is cleavable by a lysosomal cysteine endopeptidase, such as legumain. An exemplary monopeptide that may be cleaved by legumain is asparagine (Asn). Another exemplary monopeptide that may be cleaved by legumain is aspartic acid (Asp).

In some embodiments, the linker or the cleavable peptide moiety in the linker comprises an amino acid unit. In some embodiments, the amino acid unit allows for cleavage of the linker by a protease, thereby facilitating release of the drug moiety from the ADC upon exposure to one or more intracellular proteases, such as one or more lysosomal enzymes. See, e.g., Doronina et al. (2003) Nat. Biotechnol. 21:778-84; and Dubowchik and Walker (1999) Pharm. Therapeutics 83:67-123. Exemplary amino acid units include, but are not limited to, monopeptides, dipeptides, tripeptides, tetrapeptides, and pentapeptides. Exemplary monopeptides include, but are not limited to, asparagine (Asn) and aspartic acid (Asp). Exemplary dipeptides include, but are not limited to, valine-citrulline (Val-Cit), alanine-asparagine (Ala-Asn), alanine-phenylalanine (Ala-Phe), phenylalanine-lysine (Phe-Lys), alanine-lysine (Ala-Lys), alanine-valine (Ala-Val), valine-alanine (Val-Ala), valine-lysine (Val-Lys), lysine-lysine (Lys-Lys), phenylalanine-citrulline (Phe-Cit), leucine-citrulline (Leu-Cit), isoleucine-citrulline (Ile-Cit), tryptophan-citrulline (Trp-Cit), and phenylalanine-alanine (Phe-Ala). Exemplary tripeptides include, but are not limited to, alanine-alanine-asparagine (Ala-Ala-Asn), glycine-valine-citrulline (Gly-Val-Cit), glutamic acid-valine-citrulline, glycine-glycine-glycine (Gly-Gly-Gly), phenylalanine-phenylalanine-lysine (Phe-Phe-Lys), alanine-phenylalanine-lysine (Ala-Phe-Lys), glycine-valine-alanine (Gly-Val-Ala), and glycine-phenylalanine-lysine (Gly-Phe-Lys). Exemplary tetrapeptides include, but are not limited to, glycine-glycine-phenylalanine-glycine (Gly-Gly-Phe-Gly (SEQ ID NO: 141)). Other exemplary amino acid units include, but are not limited to, Gly-Phe-Leu-Gly (SEQ ID NO: 142), Ala-Leu-Ala-Leu (SEQ ID NO: 143), Phe-N9-tosyl-Arg, and Phe-N9-Nitro-Arg, as described in, e.g., U.S. Pat. No. 6,214,345. In some embodiments, an amino acid unit may comprise amino acid residues comprising at least one methyl group, e.g., a monomethyl or dimethyl group. Exemplary amino acid units that comprise amino acid residues comprising at least one methyl group include, but are not limited to, N-methylated alanine ((NMe)Ala), methylated aspartic acid (Asp(OMe)) and dimethylated lysine (Val-Lys(Me)₂). In some embodiments, the amino acid unit in the linker comprises Val-Ala. In some embodiments, the amino acid unit in the linker comprises Val-Cit. An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, a lysosomal protease such as legumain or cathepsin B, C, D, or S.

In some embodiments, the linker in an ADC disclosed herein may comprise an antibody attachment moiety. An antibody attachment moiety may be used, for example, to link the antibody moiety to the linker, which in turn may link to the drug moiety, e.g., indirectly through a cleavable moiety (e.g., a cleavable peptide).

In some embodiments, the linker comprises an antibody attachment moiety comprising a maleimide moiety (Mal). The term “maleimide moiety,” as used herein, means a compound that contains a maleimide group and that is reactive with a sulfhydryl group, e.g., a sulfhydryl group of a cysteine residue on the antibody moiety. Other functional groups that are reactive with sulfhydryl groups (thiols) and may therefore be used in place of a Mal include, but are not limited to, iodoacetamide, bromoacetamide, vinyl pyridine, disulfide, pyridyl disulfide, isocyanate, and isothiocyanate.

In some embodiments, the linker attaches to the antibody or antigen-binding fragment via a Mal moiety. In some embodiments, the Mal moiety is reactive with a cysteine residue on the antibody or antigen-binding fragment. In some embodiments, the Mal moiety is joined to the antibody or antigen-binding fragment via the cysteine residue.

In some embodiments, the Mal moiety is a maleimidocaproyl (MC) moiety. In some embodiments, the linker attaches to the antibody or antigen-binding fragment via an MC moiety. In some embodiments, the MC moiety is reactive with a cysteine residue on the antibody or antigen-binding fragment. In some embodiments, the MC moiety is joined to the antibody or antigen-binding fragment via the cysteine residue.

In some embodiments, the linker comprises a Mal moiety and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the Mal moiety attaches the antibody moiety to the cleavable peptide moiety in the linker. In some embodiments, the cleavable peptide moiety comprises an amino acid unit. In some embodiments, the amino acid unit comprises Val-Cit. In some embodiments, the amino acid unit comprises Val-Ala. In some embodiments, the linker comprises Mal-Val-Cit. In some embodiments, the linker comprises Mal-Val-Ala.

In some embodiments, the linker comprises an MC moiety and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the MC moiety attaches the antibody moiety to the cleavable peptide moiety in the linker. In some embodiments, the cleavable peptide moiety comprises an amino acid unit. In some embodiments, the amino acid unit comprises Val-Cit. In some embodiments, the amino acid unit comprises Val-Ala. In some embodiments, the linker comprises MC-Val-Cit. In some embodiments, the linker comprises MC-Val-Ala.

In some embodiments, any of the linkers in ADCs disclosed herein may comprise at least one spacer unit joining the antibody moiety to the drug moiety. In some embodiments, the spacer unit joins a cleavage site (e.g., a cleavable peptide moiety) in the linker to the antibody moiety. In some embodiments, the spacer unit joins a cleavage site (e.g., a cleavable peptide moiety) in the linker to the drug moiety. In some embodiments, the linker, and/or spacer unit in the linker, is substantially hydrophilic. In some embodiments, the linker includes one or more polyethylene glycol (PEG) moieties, e.g., 1, 2, 3, or 4 PEG moieties. In some embodiments, the linker includes one or more alkyl moieties, e.g., 1, 2, 3, 4, or 5 alkyl moieties.

In some embodiments, the spacer unit in the linker comprises one or more PEG moieties. In some embodiments, the spacer unit comprises -(PEG)ₘ-, and m is an integer from 1 to 10. In some embodiments, m ranges from 1 to 4; or from 2 to 4. In some embodiments, m is 2. In some embodiments, m is 3. In some embodiments, m is 4. In some embodiments, the spacer unit comprises (PEG)₂, (PEG)₃, or (PEG)₄. In some embodiments, the spacer unit comprises PEG₂-Lys(ε-PEG₈-OMe)-PEG₂.

In some embodiments, the spacer unit in the linker comprises an alkyl moiety. In some embodiments, the spacer unit comprises —(CH₂)ₙ—, and n is an integer from 1 to 10 (i.e., n may be 1, 2, 3, 4, 5, 6, 7, 8, 9, or 10). In some embodiments, n is 3, 4, or 5. In some embodiments, the spacer unit comprises (CH₂)₃, or (CH₂)₄, or (CH₂)₅. In some embodiments, the spacer unit comprises CH₂—CH₂.

In some embodiments, the spacer unit comprises

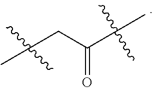

In some embodiments, the spacer unit comprises

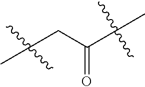 [SMILES: *CC(*)=O]

and (PEG)₂. In some embodiments, the spacer unit comprises

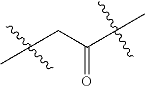 [SMILES: *CC(*)=O]

conjugated to (PEG)₂. In some embodiments, the spacer unit comprises

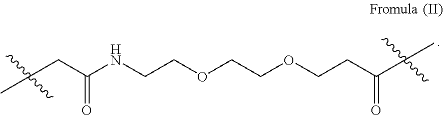

In some embodiments, linkers disclosed herein may be used in L-D constructs with other D moieties. In some embodiments, using a linker comprising a spacer unit comprising Formula (II) may provide benefits for various D moieties, including, e.g., improved conjugation stability, improved plasma stability, and/or in vivo anti-tumor activity compared to other linkers comprising alternative spacer units. In some embodiments, without being bound by theory, benefits of using a linker comprising Formula (II) with a STING agonist disclosed herein, e.g., a compound of Formula (III), Formula (IV), or Table 14, e.g., Compound 1, may include improved conjugation stability, improved plasma stability, and/or in vivo anti-tumor activity. In some embodiments, a linker comprising Formula (II) and a payload comprising a STING agonist disclosed herein, e.g., a compound of Formula (III), Formula (IV), or Table 14 demonstrates superior properties when conjugated to an anti-PSMA antibody disclosed herein. Exemplary evidence of the superior benefits of such L-D and antibody-drug conjugates are shown in Examples 4, 9, 12, and 15.

A spacer unit may be used, for example, to link the antibody moiety to the drug moiety, either directly or indirectly. In some embodiments, the spacer unit links the antibody moiety to the drug moiety directly. In some embodiments, the antibody moiety and the drug moiety are attached via a spacer unit comprising one or more alkyl moieties (e.g., (CH₂)₃, or (CH₂)₄, or (CH₂)₅). In some embodiments, the antibody moiety and the drug moiety are attached via a spacer unit comprising one or more PEG moieties (e.g., (PEG)₂ or (PEG)₃ or (PEG)₄). In some embodiments, the antibody moiety and the drug moiety are attached via a spacer unit comprising Formula (II). In some embodiments, the spacer unit links the antibody moiety to the drug moiety indirectly. In some embodiments, the spacer unit links the antibody moiety to the drug moiety indirectly through a cleavable moiety (e.g., a cleavable peptide) and/or an antibody attachment moiety to join the spacer unit to the antibody moiety, e.g., a maleimide moiety or a carbobenzoxy-L-glutaminyl-glycine moiety.

In some embodiments, the spacer unit attaches to the antibody moiety (i.e., the antibody or antigen-binding fragment) via a maleimide moiety (Mal). A spacer unit that attaches to the antibody or antigen-binding fragment via a Mal is referred to herein as a “Mal-spacer unit.” In some embodiments, the Mal-spacer unit is reactive with a cysteine residue on the antibody or antigen-binding fragment. In some embodiments, the Mal-spacer unit is joined to the antibody or antigen-binding fragment via the cysteine residue. In some embodiments, the Mal-spacer unit comprises a PEG moiety. In some embodiments, the Mal-spacer unit comprises an alkyl moiety. In some embodiments, the Mal-spacer unit comprises Formula (II). In some embodiments, the spacer unit attaches to the antibody moiety (i.e., the antibody or antigen-binding fragment) via a maleimidocaproyl moiety (MC). A spacer unit that attaches to the antibody or antigen-binding fragment via an MC is referred to herein as an “MC-spacer unit.” In some embodiments, the MC-spacer unit is reactive with a cysteine residue on the antibody or antigen-binding fragment. In some embodiments, the MC-spacer unit is joined to the antibody or antigen-binding fragment via the cysteine residue. In some embodiments, the MC-spacer unit comprises a PEG moiety. In some embodiments, the MC-spacer unit comprises an alkyl moiety. In some embodiments, the MC-spacer unit comprises Formula (II).

In some embodiments, the linker comprises the Mal-spacer unit or MC-spacer unit and a cleavable peptide moiety. In some embodiments, the cleavable peptide moiety comprises an amino acid unit. In some embodiments, the amino acid unit comprises Val-Cit. In some embodiments, the amino acid unit comprises Val-Ala. In some embodiments, the linker comprises the Mal-spacer unit or MC-spacer unit and an amino acid unit. In some embodiments, the linker comprises Mal-(CH₂)ₙ and an amino acid unit, where n is 3 to 5, or 3, 4, or 5. In some embodiments, the linker comprises MC-(CH₂)ₙ and an amino acid unit, where n is 3 to 5, or 3, 4, or 5.

In some embodiments, the linker comprises Mal-(PEG)ₘ and an amino acid unit, where m is 2 to 4, or 2, 3, or 4. In some embodiments, the linker comprises MC-(PEG)ₘ and an amino acid unit, where m is 2 to 4, or 2, 3, or 4. In some embodiments, the linker further comprises a cleavable dipeptide, e.g., Val-Cit or Val-Ala. In some embodiments, the linker comprises Mal-(PEG)ₙ-Val-Cit, where n is any number between 1 and 10. In some embodiments, the linker comprises Mal-(PEG)ₙ-Val-Ala, where n is any number between 1 and 10. In some embodiments, the linker comprises MC-(PEG)ₙ-Val-Cit, where n is any number between 1 and 10. In some embodiments, the linker comprises MC-(PEG)ₙ-Val-Ala, where n is any number between 1 and 10.

In some embodiments, the linker comprises Mal-Formula (II) and an amino acid unit. In some embodiments, the linker comprises a cleavable dipeptide, e.g., Val-Cit or Val-Ala. In some embodiments, the linker comprises Mal-Formula (II)-Val-Cit. In some embodiments, the linker comprises Mal-Formula (II)-Val-Ala.

In some embodiments, the Mal-spacer unit or MC-spacer unit attaches the antibody moiety (i.e., the antibody or antigen-binding fragment) to the cleavable moiety in the linker. In some embodiments, the Mal-spacer unit or MC-spacer unit attaches the antibody or antigen-binding fragment to a cleavable peptide moiety. In some embodiments, the cleavable peptide moiety comprises an amino acid unit. In some embodiments, the linker comprises Mal-spacer unit-amino acid unit. In some embodiments, the Mal-spacer unit comprises a PEG moiety. In some embodiments, the Mal-spacer-unit comprises an alkyl moiety. In some embodiments, the Mal-spacer unit comprises Formula (II). In some embodiments, the linker comprises MC-spacer unit-amino acid unit. In some embodiments, the MC-spacer unit comprises a PEG moiety. In some embodiments, the MC-spacer unit comprises an alkyl moiety.

In various embodiments, the cleavable moiety in the linker is joined directly to the drug moiety and/or to the antibody moiety. In other embodiments, a spacer unit is used to attach the cleavable moiety in the linker to the drug moiety and/or to the antibody moiety. In various embodiments, the drug moiety may be any STING agonist drug moiety disclosed herein, e.g., a compound of Formula (III), Formula (IV), or a compound disclosed in Table 14, infra. In various embodiments, the drug moiety is attached to the cleavable moiety in the linker by a spacer unit. In various embodiments, the drug moiety is Compound 1. In various embodiments, the Compound 1 moiety is attached to the cleavable moiety in the linker by a spacer unit. In some embodiments, the drug moiety, e.g., Compound 1, is attached to the cleavable moiety in the linker by a self-immolative unit. In some embodiments, the drug moiety, e.g., Compound 1, is attached to the cleavable moiety in the linker by a self-immolative unit, the cleavable moiety comprises an amino acid unit, and a further spacer unit, e.g., comprising one or more alkyl or PEG moieties or Formula (II), joins the cleavable moiety to the antibody moiety. In some embodiments, the drug moiety, e.g., Compound 1, is joined to an anti-PSMA antibody via a Mal-spacer unit in the linker joined to a cleavable peptide moiety and a pAB or pABC self-immolative unit. In some embodiments, the drug moiety, e.g., Compound 1, is joined to an anti-PSMA antibody via an MC-spacer unit in the linker joined to a cleavable peptide moiety and a pAB or pABC self-immolative unit.

A spacer unit may be “self-immolative” or “non-self-immolative.” A “non-self-immolative” spacer unit is one in which part or all of the spacer unit remains bound to the drug moiety upon cleavage of the linker. Examples of non-self-immolative units include, but are not limited to, a glycine spacer unit and a glycine-glycine spacer unit. Non-self-immolative units may eventually degrade over time but do not readily release a linked native drug entirely under cellular conditions.

A “self-immolative” unit comprises any structure that allows for release of the native drug moiety after administration to a subject, e.g., under intracellular conditions. A “native drug” is one where no part of the spacer unit or other chemical modification remains after cleavage/degradation of the spacer unit.

Self-immolation chemistry is known in the art and may be readily selected for the disclosed ADCs. In various embodiments, the spacer unit attaching the cleavable moiety in the linker to the drug moiety (e.g., Compound 1) is self-immolative, and undergoes self-immolation concurrently with or shortly before/after cleavage of the cleavable moiety under intracellular conditions.

In various embodiments, a linker disclosed herein may comprise at least one self-immolative unit. Any of the linkers disclosed herein may comprise a first self-immolative unit. The phrase “first self-immolative unit” may indicate a linker comprising one self-immolative unit or a linker comprising one or more self-immolative units. In some embodiments, a linker disclosed herein comprises a first self-immolative unit and a second self-immolative unit.

In certain embodiments, the at least one self-immolative unit in the linker comprises a p-aminobenzyl unit. In some embodiments, a p-aminobenzyl alcohol (pABOH) is attached to an amino acid unit or other cleavable moiety in the linker via an amide bond, and a carbamate, methylcarbamate, or carbonate is made between the pABOH and the drug moiety. See, e.g., Hamann et al. (2005) Expert Opin. Ther. Patents 15:1087-103. In some embodiments, the at least one self-immolative unit is or comprises p-aminobenzyl (pAB). In some embodiments, the at least one self-immolative unit is or comprises p-aminobenzyloxycarbonyl (pABC). Without being bound by theory, it is thought that the self-immolation of pAB or pABC involves a spontaneous 1,6-elimination reaction. See, e.g., Jain et al. (2015) Pharm Res 32:3526-40.

In various embodiments, the structure of the p-aminobenzyl (pAB) used in the disclosed ADCs is shown below:

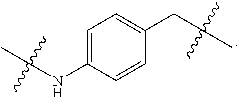

In various embodiments, the structure of the p-aminobenzyloxycarbonyl (pABC) used in the disclosed ADCs is shown below:

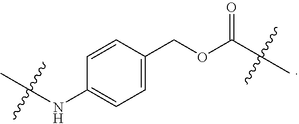

The structure of the pAB or pABC in a self-immolative unit may be substituted.

In some embodiments, the pAB is substituted with 1-3 substituents chosen from methyl, fluoro, chloro, trifluoromethyl, C₆-C₁₀ aryl, and C₅-C₁₂ heteroaryl. Exemplary substituted pAB units are disclosed in Table 12. In some embodiments, a linker disclosed herein may comprise a self-immolative unit selected from the self-immolative units disclosed in Table 12, infra.

TABLE 12
Exemplary Substituted pAB Moieties

| Substituted pAB | | |
|---|---|---|
| Moiety | Substituted pAB Moiety | Structure |
| (N—Me)pAB | N-methyl-p-aminobenzyl | 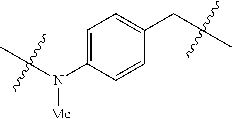 [SMILES: *Cc1ccc(N(*)C)cc1] |
| MAB | m-aminobenzyl | 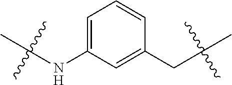 [SMILES: *Cc1cccc(N*)c1] |
| OAB | o-aminobenzyl | 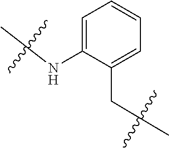 [SMILES: *Cc1ccccc1N*] |
| (2-Cl)pAB | 2-chloro-4-aminobenzyl | 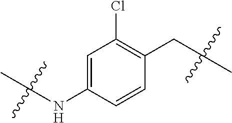 [SMILES: *Cc1ccc(N*)cc1Cl] |
| (3-Me)pAB | 3-methyl-4-aminobenzyl | 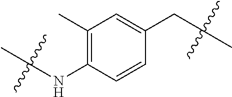 [SMILES: *Cc1ccc(N*)c(C)c1] |
| (3-Cl)pAB | 3-chloro-4-aminobenzyl | 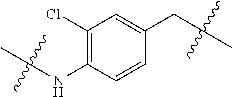 [SMILES: *Cc1ccc(N*)c(Cl)c1] |
| (3-F)pAB | 3-fluoro-4-aminobenzyl | 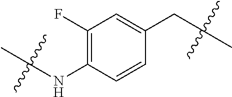 [SMILES: *Cc1ccc(N*)c(F)c1] |
| (2,5-Cl2)pAB | 2,5-dichloro-4-aminobenzyl | 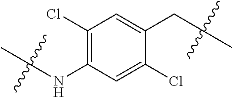 [SMILES: *Cc1cc(Cl)c(N*)cc1Cl] |
| (3-CF₃)pAB | 3-trifluoromethyl-4-aminobenzyl | 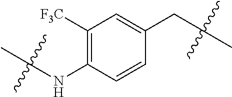 [SMILES: *Cc1ccc(N*)c(C(F)(F)F)c1] |
| (2-F)pAB | 2-fluoro-4-aminobenzyl | 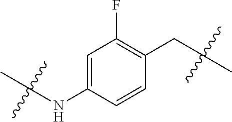 [SMILES: *Cc1ccc(N*)cc1F] |
| (3-MeO)pAB | 3-methoxy-4-aminobenzyl | 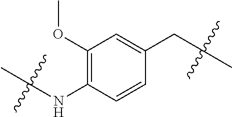 [SMILES: *Cc1ccc(N*)c(OC)c1] |
| (2,5-Me2)pAB | 2,5-dimethyl-4-aminobenzyl | 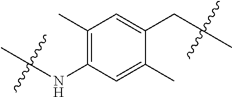 [SMILES: *Cc1cc(C)c(N*)cc1C] |
| (2,6-Cl2)pAB | 2,6-dichloro-4-aminobenzyl | 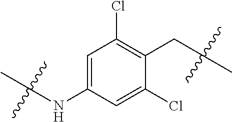 [SMILES: *Cc1c(Cl)cc(N*)cc1Cl] |
| (2-Me)pAB | 2-methyl-4-aminobenzyl | 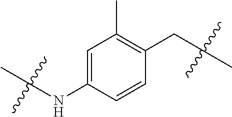 [SMILES: *Cc1ccc(N*)cc1C] |
| (2Py)pAB | (5-aminopyridin-2-yl)methyl | 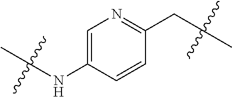 [SMILES: *Cc1ccc(N*)cn1] |

Linker moieties may be modified to achieve desirable properties of an ADC, e.g., stability, tolerability, and/or efficacy. For example, a linker comprising a modified pAB or pABC moiety may increase ADC stability and/or in vivo ADC tolerability (as determined by, for example, percent body weight loss) while minimizing reduced ADC efficacy when compared to a linker comprising pAB or pABC. Certain additional modifications to the linker-drug structure, e.g., spacer units or modified drug moiety attachment points, may be required to obtain one or more (e.g., all of these) properties. For instance, certain modifications or combinations of modifications may need to be made to enhance ADC stability while avoiding loss of efficacy. For example, in some embodiments, an ADC comprising LP1, LP2, LP16, LP20, LP26, or LP28 may achieve desirable properties of an ADC, e.g., stability, tolerability, and/or efficacy when compared to other anti-PSMA ADCs.

In some embodiments, any of the linkers disclosed herein may comprise a further self-immolative unit. In some embodiments, the further self-immolative unit attaches the first self-immolative unit to the drug moiety (e.g., Compound 1). The addition of one or more further self-immolative unit(s) to a linker-payload conjugate as disclosed herein may provide superior technical benefits, e.g., superior stability and/or improved activity, compared to other linker-payload conjugates comprising any of the payload compounds disclosed herein. Any of the linkers disclosed herein may comprise a second self-immolative unit.

Exemplary additional self-immolative units are disclosed in Table 13. In some embodiments, a linker-payload conjugate comprises a second self-immolative unit listed in Table 13, infra. In some embodiments, a linker-payload conjugate comprises Val-Ala-pAB and a second self-immolative unit selected from Table 13. In some embodiments, a linker-payload conjugate comprises Val-Ala-pABC and a second self-immolative unit selected from Table 13. In some embodiments, a linker-payload conjugate comprises Val-Cit-pAB and a second self-immolative unit selected from Table 13. In some embodiments, a linker-payload conjugate comprises Val-Cit-pABC and a second self-immolative unit selected from Table 13.

TABLE 13
Exemplary Self-Immolative Units

| Second Self- | | |
|---|---|---|
| immolative Unit | Chemical Name | Chemical Structure |
| Unit 1 (MEC) | (N-methylamino)ethoxycarbonyl | 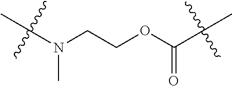 [SMILES: *C(=O)OCCN(*)C] |
| Unit 2 | (N-methylamino)-2- cyclopropyl]methoxycarbonyl | 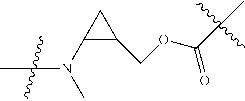 [SMILES: *C(=O)OCC1CC1N(*)C] |
| Unit 3 | 3-(N-methylamino)butanoyl | 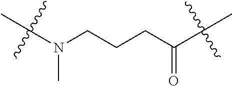 [SMILES: *C(=O)CCCN(*)C] |
| Unit 4 | N-methylamino-(cyclopropyl)-1- methoxycarbonyl | 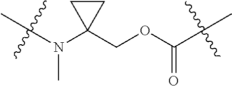 [SMILES: *C(=O)OCC1(N(*)C)CC1] |
| Unit 5 | (N-methylamino)-1,1-dimethyl-ethoxycarbonyl | 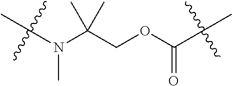 [SMILES: *C(=O)OCC(C)(C)N(*)C] |
| Unit 6 | N-methylamino-(cyclopropyl)-2- methoxycarbonyl | 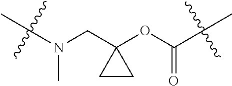 [SMILES: *C(=O)OC1(CN(*)C)CC1] |
| Unit 7 | (N—H)ethoxycarbonyl | 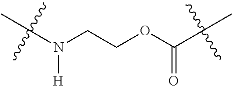 [SMILES: *NCCOC(*)=O] |
| Unit 8 | 2-(N-methyl)aminomethylbenzoate | 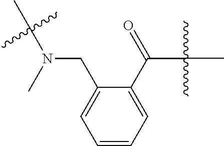 [SMILES: *C(=O)c1ccccc1CN(*)C] |
| Unit 9 | prolinolcarbonyl | 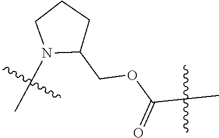 [SMILES: *C(=O)OCC1CCCN1*] |
| Unit 10 | 4-monofluoro-prolinolcarbonyl | 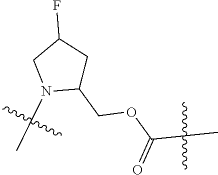 [SMILES: *C(=O)OCC1CC(F)CN1*] |
| Unit 11 | 4,4-difluoroprolinolcarbonyl | 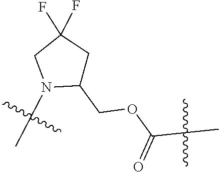 [SMILES: *C(=O)OCC1CC(F)(F)CN1*] |
| Unit 12 | 3-monofluoro-prolinolcarbonyl | 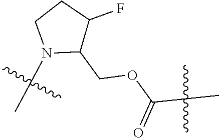 [SMILES: *C(=O)OCC1C(F)CCN1*] |
| Unit 13 | 3,3-difluoroprolinolcarbonyl | 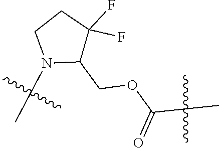 [SMILES: *C(=O)OCC1N(*)CCC1(F)F] |

Units 2 and 9-13 include all stereoisomers.

In some embodiments, the further self-immolative unit comprises a Unit 1 (MEC) moiety. In some embodiments, a MEC moiety attaches the first self-immolative unit to the drug moiety (e.g., Compound 1) (“self-immolative unit-MEC moiety”). In some embodiments, the further self-immolative unit comprises a Unit 2 moiety. In some embodiments, a Unit 2 moiety attaches the first self-immolative unit to the drug moiety (e.g., Compound 1) (“self-immolative unit-Unit 2 moiety”). In some embodiments, the further self-immolative unit comprises a Unit 3 moiety. In some embodiments, a Unit 3 moiety attaches the first self-immolative unit to the drug moiety (e.g., Compound 1) (“self-immolative unit-Unit 3 moiety”). In some embodiments, the further self-immolative unit comprises a Unit 4 moiety. In some embodiments, a Unit 4 moiety attaches the first self-immolative unit to the drug moiety (e.g., Compound 1) (“self-immolative unit-Unit 4 moiety”). In some embodiments, the further self-immolative unit comprises a Unit 5 moiety. In some embodiments, a Unit 5 moiety attaches the first self-immolative unit to the drug moiety (e.g., Compound 1) (“self-immolative unit-Unit 5 moiety”). In some embodiments, the further self-immolative unit comprises a Unit 6 moiety. In some embodiments, a Unit 6 moiety attaches the first self-immolative unit to the drug moiety (e.g., Compound 1) (“self-immolative unit-Unit 6 moiety”). In some embodiments, the further self-immolative unit comprises a Unit 7 moiety. In some embodiments, a Unit 7 moiety attaches the first self-immolative unit to the drug moiety (e.g., Compound 1) (“self-immolative unit-Unit 7 moiety”). In some embodiments, the further self-immolative unit comprises a Unit 8 moiety. In some embodiments, a Unit 8 moiety attaches the first self-immolative unit to the drug moiety (e.g., Compound 1) (“self-immolative unit-Unit 8 moiety”). In some embodiments, the further self-immolative unit comprises a Unit 9 moiety. In some embodiments, a Unit 9 moiety attaches the first self-immolative unit to the drug moiety (e.g., Compound 1) (“self-immolative unit-Unit 9 moiety”). In some embodiments, the further self-immolative unit comprises a Unit 10 moiety. In some embodiments, a Unit 10 moiety attaches the first self-immolative unit to the drug moiety (e.g., Compound 1) (“self-immolative unit-Unit 10 moiety”). In some embodiments, the further self-immolative unit comprises a Unit 11 moiety. In some embodiments, a Unit 11 moiety attaches the first self-immolative unit to the drug moiety (e.g., Compound 1) (“self-immolative unit-Unit 11 moiety”). In some embodiments, the further self-immolative unit comprises a Unit 12 moiety. In some embodiments, a Unit 12 moiety attaches the first self-immolative unit to the drug moiety (e.g., Compound 1) (“self-immolative unit-Unit 12 moiety”). In some embodiments, the further self-immolative unit comprises a Unit 13 moiety. In some embodiments, a Unit 13 moiety attaches the first self-immolative unit to the drug moiety (e.g., Compound 1) (“self-immolative unit-Unit 13 moiety”).

In various embodiments, a cleavable moiety in a linker attaches directly or indirectly to a sulfur in the drug moiety. The drug moiety may be any suitable drug moiety disclosed herein, e.g., a compound of Formula (III), Formula (IV), or a compound disclosed in Table 14, infra. In some embodiments, the drug moiety is or comprises Compound 1. In some embodiments, the cleavable moiety in the linker attaches directly or indirectly to the S-14 sulfur in a STING agonist drug moiety disclosed herein (e.g., Compound 1). In some embodiments, the one or more self-immolative unit(s) comprises pAB. In some embodiments, the pAB attaches the cleavable moiety in the linker to the S-14 sulfur in a STING agonist drug moiety disclosed herein (e.g., Compound 1). In some embodiments, the pAB undergoes self-immolation upon cleavage of the cleavable moiety, and the STING agonist drug moiety (e.g., Compound 1) is released from the ADC in its native, active form. In some embodiments, the cleavable moiety comprises an amino acid unit. In some embodiments, the linker comprises amino acid unit-pAB. In some embodiments, the amino acid unit is Val-Cit. In some embodiments, the linker comprises Val-Cit-pAB. In some embodiments, the amino acid unit is Val-Ala. In some embodiments, the linker comprises Val-Ala-pAB.

In various embodiments, a cleavable moiety in the linker attaches directly or indirectly to a nitrogen in the drug moiety. The drug moiety may be a STING agonist drug moiety disclosed herein, e.g., a compound of Formula (III), Formula (IV), or a compound disclosed in Table 14, infra. In some embodiments, the drug moiety is or comprises Compound 1. In some embodiments, the nitrogen in the STING agonist drug moiety (e.g., Compound 1) is the N-34 nitrogen. In some embodiments, the nitrogen in the STING agonist drug moiety (e.g., Compound 1) is the N-39 nitrogen. In some embodiments, the one or more self-immolative unit(s) comprises pAB. In some embodiments, the one or more self-immolative unit(s) comprises pABC. In some embodiments, the one or more self-immolative unit(s) comprises a MEC moiety. In some embodiments, the one or more self-immolative unit(s) comprises pABC-MEC moiety. In some embodiments, the carboxylate moiety of the pABC is bound to the n-methyl moiety of the MEC to form an N-methylcarbamate moiety. In some embodiments, the one or more self-immolative unit(s) comprises a Unit 8 moiety. In some embodiments, the one or more self-immolative unit(s) comprises pABC-Unit 8 moiety. In some embodiments, the one or more self-immolative unit(s) comprises a Unit 9 moiety. In some embodiments, the one or more self-immolative unit(s) comprises pABC-Unit 9 moiety. In some embodiments, the one or more self-immolative unit(s) comprises a Unit 11 moiety. In some embodiments, the one or more self-immolative unit(s) comprises pABC-Unit 11 moiety.

In some embodiments, the linker comprises a third spacer unit between the first spacer unit and the second spacer unit. In some embodiments, the second and/or third spacer unit is selected from a moiety of Table 13, supra. In some embodiments, the linker comprises a third spacer unit between the pABC spacer unit and the MEC spacer unit. In some embodiments, the linker comprises a third spacer unit between the pABC spacer unit and the Unit 8 spacer unit. In some embodiments, the linker comprises a third spacer unit between the pABC spacer unit and the Unit 9 spacer unit. In some embodiments, the linker comprises a third spacer unit between the pABC spacer unit and the Unit 11 spacer unit. In some embodiments, the pABC attaches the cleavable moiety in the linker to the N-34 nitrogen in Compound 1. In some embodiments, the pABC attaches the cleavable moiety in the linker to the N-39 nitrogen in Compound 1.

In some embodiments, the pABC-MEC moiety attaches the cleavable moiety in the linker to the N-34 nitrogen in Compound 1. In some embodiments, the pABC-MEC moiety attaches the cleavable moiety in the linker to the N-39 nitrogen in Compound 1. In some embodiments, the pABC or pABC-MEC moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the MEC moiety undergoes self-immolation. In some embodiments, the cleavable moiety comprises an amino acid unit. In some embodiments, the linker comprises amino acid unit-pABC. In some embodiments, the linker comprises amino acid unit-pABC-MEC moiety. In some embodiments, the amino acid unit is Val-Cit. In some embodiments, the linker comprises Val-Cit-pABC. In some embodiments, the linker comprises Val-Cit-pABC-MEC moiety. In some embodiments, the amino acid unit is Val-Ala. In some embodiments, the linker comprises Val-Ala-pABC. In some embodiments, the linker comprises Val-Ala-pABC-MEC moiety.

In some embodiments, the pABC-Unit 8 moiety attaches the cleavable moiety in the linker to the N-34 nitrogen in Compound 1. In some embodiments, the pABC-Unit 8 moiety attaches the cleavable moiety in the linker to the N-39 nitrogen in Compound 1. In some embodiments, the pABC or pABC-Unit 8 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 8 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 8 moiety. In some embodiments, the amino acid unit is Val-Cit. In some embodiments, the linker comprises Val-Cit-pABC. In some embodiments, the linker comprises Val-Cit-pABC-Unit 8 moiety. In some embodiments, the amino acid unit is Val-Ala. In some embodiments, the linker comprises Val-Ala-pABC. In some embodiments, the linker comprises Val-Ala-pABC-Unit 8 moiety.

In some embodiments, the pABC-Unit 9 moiety attaches the cleavable moiety in the linker to the N-34 nitrogen in Compound 1. In some embodiments, the pABC-Unit 9 moiety attaches the cleavable moiety in the linker to the N-39 nitrogen in Compound 1. In some embodiments, the pABC or pABC-Unit 9 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 9 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 9 moiety. In some embodiments, the amino acid unit is Val-Cit. In some embodiments, the linker comprises Val-Cit-pABC. In some embodiments, the linker comprises Val-Cit-pABC-Unit 9 moiety. In some embodiments, the amino acid unit is Val-Ala. In some embodiments, the linker comprises Val-Ala-pABC. In some embodiments, the linker comprises Val-Ala-pABC-Unit 9 moiety.

In some embodiments, the pABC-Unit 11 moiety attaches the cleavable moiety in the linker to the N-34 nitrogen in Compound 1. In some embodiments, the pABC-Unit 11 moiety attaches the cleavable moiety in the linker to the N-39 nitrogen in Compound 1. In some embodiments, the pABC or pABC-Unit 11 moiety undergoes self-immolation upon cleavage of the cleavable moiety, and Compound 1 is released from the ADC in its native, active form. In some embodiments, the release of Compound 1 from the antibody and linker occurs in a stepwise fashion, wherein first the cleavable moiety in the linker is cleaved, then the pABC moiety undergoes self-immolation, and then the Unit 11 moiety undergoes self-immolation. In some embodiments, the cleavable moiety comprises an amino acid unit. In some embodiments, the linker comprises amino acid unit-pABC. In some embodiments, the linker comprises amino acid unit-pABC-Unit 11 moiety. In some embodiments, the amino acid unit is Val-Cit. In some embodiments, the linker comprises Val-Cit-pABC. In some embodiments, the linker comprises Val-Cit-pABC-Unit 11 moiety. In some embodiments, the amino acid unit is Val-Ala. In some embodiments, the linker comprises Val-Ala-pABC. In some embodiments, the linker comprises Val-Ala-pABC-Unit 11 moiety.

In some embodiments, the at least one self-immolative unit (e.g., pAB, pABC, pABC-MEC moiety, pABC-Unit 8 moiety, pABC-Unit 9 moiety, or pABC-Unit 11 moiety) undergoes self-immolation upon cleavage of a cleavable peptide moiety in the linker. In some embodiments, the self-immolation of the at least one self-immolative unit (e.g., pAB, pABC, pABC-MEC moiety, pABC-Unit 8 moiety, pABC-Unit 9 moiety, or pABC-Unit 11 moiety) occurs in a stepwise manner after cleavage of a cleavable peptide moiety in the linker, starting from the self-immolative moiety closest to the cleavable peptide moiety. In some embodiments, the at least one self-immolative unit (e.g., pAB, pABC, pABC-MEC moiety, pABC-Unit 8 moiety, pABC-Unit 9 moiety, or pABC-Unit 11 moiety) undergoes self-immolation in a stepwise manner after cleavage of a cleavable peptide moiety in the linker, wherein the first self-immolative unit (e.g., pABC or pAB) undergoes self-immolation prior to self-immolation of the second self-immolative unit (e.g., MEC moiety, Unit 8 moiety, Unit 9 moiety, Unit 11 moiety). In some embodiments, the cleavable peptide moiety comprises an amino acid unit.

In some embodiments, the linker comprises amino acid unit-pAB. In some embodiments, the linker comprises amino acid unit-pABC. In some embodiments, the linker comprises amino acid unit-pABC-MEC moiety. In some embodiments, the linker comprises amino acid unit-pABC-Unit 8 moiety. In some embodiments, the linker comprises amino acid unit-pABC-Unit 9 moiety. In some embodiments, the linker comprises amino acid unit-pABC-Unit 11 moiety. In some embodiments, the amino acid unit is Val-Cit. In some embodiments, the linker comprises Val-Cit-pAB. In some embodiments, the linker comprises Val-Cit-pABC. In some embodiments, the linker comprises Val-Cit-pABC-MEC moiety. In some embodiments, the linker comprises Val-Cit-pABC-Unit 8 moiety. In some embodiments, the linker comprises Val-Cit-pABC-Unit 9 moiety. In some embodiments, the linker comprises Val-Cit-pABC-Unit 11 moiety. In some embodiments, the amino acid unit is Val-Ala.

In some embodiments, the linker comprises Val-Ala-pAB. In some embodiments, the linker comprises Val-Ala-pABC. In some embodiments, the linker comprises Val-Ala-pABC-MEC moiety. In some embodiments, the linker comprises Val-Ala-pABC-Unit 8 moiety. In some embodiments, the linker comprises Val-Ala-pABC-Unit 9 moiety. In some embodiments, the linker comprises Val-Ala-pABC-Unit 11 moiety.

In various aspects, the antibody moiety of the ADC is conjugated to the drug moiety via a linker, wherein the linker comprises an MC-spacer unit, a cleavable amino acid unit, and a pAB. In some embodiments, the linker comprises MC-Val-Cit-pAB. In some embodiments, the linker comprises MC-Val-Ala-pAB.

In various aspects, the antibody moiety of the ADC is conjugated to the drug moiety via a linker, wherein the linker comprises an MC-spacer unit, a cleavable amino acid unit, and a pABC. In some embodiments, the linker comprises MC-Val-Cit-pABC. In some embodiments, the linker comprises MC-Val-Ala-pABC.

In various aspects, the antibody moiety of the ADC is conjugated to the drug moiety via a linker, wherein the linker comprises an MC unit, a cleavable amino acid unit, a pABC, and a MEC moiety. In some embodiments, the linker comprises MC-Val-Cit-pABC-MEC moiety. In some embodiments, the linker comprises MC-Val-Ala-pABC-MEC moiety.

In various aspects, the antibody moiety of the ADC is conjugated to the drug moiety via a linker, wherein the linker comprises an MC unit, a cleavable amino acid unit, a pABC, and a Unit 8 moiety. In some embodiments, the linker comprises MC-Val-Cit-pABC-Unit 8 moiety. In some embodiments, the linker comprises MC-Val-Ala-pABC-Unit 8 moiety.

In various aspects, the antibody moiety of the ADC is conjugated to the drug moiety via a linker, wherein the linker comprises an MC unit, a cleavable amino acid unit, a pABC, and a Unit 9 moiety. In some embodiments, the linker comprises MC-Val-Cit-pABC-Unit 9 moiety. In some embodiments, the linker comprises MC-Val-Ala-pABC-Unit 9 moiety.

In various aspects, the antibody moiety of the ADC is conjugated to the drug moiety via a linker, wherein the linker comprises an MC unit, a cleavable amino acid unit, a pABC, and a Unit 11 moiety. In some embodiments, the linker comprises MC-Val-Cit-pABC-Unit 11 moiety. In some embodiments, the linker comprises MC-Val-Ala-pABC-Unit 11 moiety.

In some embodiments, the antibody moiety is conjugated to the drug moiety via a linker comprising a maleimidicopryl moiety (MC) and an amino acid. In some embodiments, the antibody moiety is conjugated to the drug moiety via a linker comprising a maleimidocaproyl moiety (MC), an amino acid, and a pAB. In some embodiments, the antibody moiety is conjugated to the drug moiety via a linker comprising a maleimidocaproyl moiety (MC), an amino acid, and a pABC. In some embodiments, the antibody moiety is conjugated to the drug moiety via a linker comprising a maleimidocaproyl moiety (MC), an amino acid, a pABC, and a MEC moiety. In some embodiments, the antibody moiety is conjugated to the drug moiety via a linker comprising a maleimidocaproyl moiety (MC), an amino acid, a pABC, and a Unit 8 moiety. In some embodiments, the antibody moiety is conjugated to the drug moiety via a linker comprising a maleimidocaproyl moiety (MC), an amino acid, a pABC, and a Unit 9 moiety. In some embodiments, the antibody moiety is conjugated to the drug moiety via a linker comprising a maleimidocaproyl moiety (MC), an amino acid, a pABC, and a Unit 11 moiety.

In various aspects, the antibody moiety of the ADC is conjugated to the drug moiety via a linker, wherein the linker comprises a Mal-spacer unit, a cleavable amino acid unit, and a pAB. In some embodiments, the linker comprises Mal-Formula (II)-Val-Cit-pAB. In some embodiments, the linker comprises Mal-Formula (II)-Val-Ala-pAB. In various aspects, the antibody moiety of the ADC is conjugated to the drug moiety via a linker, wherein the linker comprises a Mal-spacer unit, a cleavable amino acid unit, and a pABC. In some embodiments, the linker comprises Mal-Formula (II)-Val-Cit-pABC. In some embodiments, the linker comprises Mal-Formula (II)-Val-Ala-pABC.

In various aspects, the antibody moiety of the ADC is conjugated to the drug moiety via a linker, wherein the linker comprises a Mal unit, a cleavable amino acid unit, a pABC, and a MEC moiety. In some embodiments, the linker comprises Mal-Formula (II)-Val-Cit-pABC-MEC moiety. In some embodiments, the linker comprises Mal-Formula (II)-Val-Ala-pABC-MEC moiety.

In various aspects, the antibody moiety of the ADC is conjugated to the drug moiety via a linker, wherein the linker comprises a Mal unit, a cleavable amino acid unit, a pABC, and a Unit 8 moiety. In some embodiments, the linker comprises Mal-Formula (II)-Val-Cit-pABC-Unit 8 moiety. In some embodiments, the linker comprises Mal-Formula (II)-Val-Ala-pABC-Unit 8 moiety.

In various aspects, the antibody moiety of the ADC is conjugated to the drug moiety via a linker, wherein the linker comprises a Mal unit, a cleavable amino acid unit, a pABC, and a Unit 9 moiety. In some embodiments, the linker comprises Mal-Formula (II)-Val-Cit-pABC-Unit 9 moiety. In some embodiments, the linker comprises Mal-Formula (II)-Val-Ala-pABC-Unit 9 moiety.

In various aspects, the antibody moiety of the ADC is conjugated to the drug moiety via a linker, wherein the linker comprises a Mal unit, a cleavable amino acid unit, a pABC, and a Unit 11 moiety. In some embodiments, the linker comprises Mal-Formula (II)-Val-Cit-pABC-Unit 11 moiety. In some embodiments, the linker comprises Mal-Formula (II)-Val-Ala-pABC-Unit 11 moiety.

In some embodiments, the drug moiety is Compound 1.

In some embodiments, the drug moiety is Compound 2.

### Drug Moieties

The drug moiety (D) of the linker-drug conjugates and ADCs disclosed herein can be any chemotherapeutic agent. In some embodiments, the drug moiety is a STING agonist. Exemplary STING agonists are known in the art and include cyclic dinucleotides, e.g., macrocycle-bridged STING agonists, non-cyclic dinucleotides. In some embodiments, the drug moiety is a non-cyclic dinucleotide. In some embodiments, the drug moiety is a macrocycle-bridged STING agonist.

The drug moiety of the linker-drug conjugates and ADCs disclosed herein comprises a compound according to one of the following Formulae:

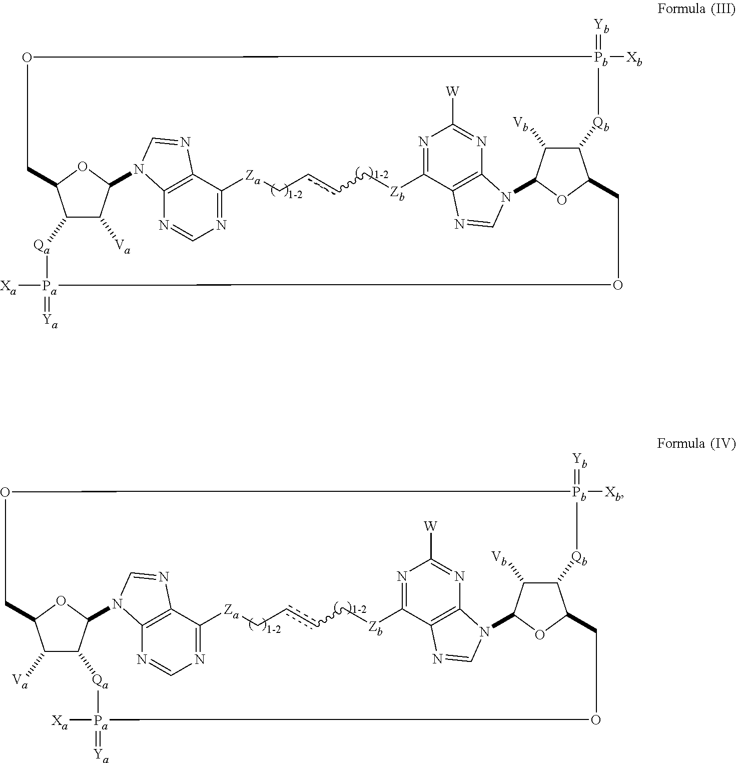

an isomer thereof, a deuterated derivative of the compound or isomer; or a salt of the compound, isomer, or deuterated derivative;
wherein, independently for each occurrence,
each of Pₐ and P_{b}, when not racemic, is independently selected from (R)-configuration and (S)-configuration;
each of Qₐ and Q_{b} is independently selected from NH and O;
each of Vₐ and V_{b} is independently selected from F and OH;
W is selected from H and NH₂;
each of Xₐ and X_{b} is independently selected from OH and SH;
each of Yₐ and Y_{b} is independently selected from O and S;
each Zₐ and Z_{b} is independently selected from CH₂, O, and NH; and
[symbol] means that the bond is selected from a single bond ( [symbol] ), a double bond ( [symbol] ) of (E)- or (Z)-configuration, or a triple bond ( [symbol] );
provided that at least one of Zₐ and Z_{b} is NH or at least one of Xₐ and X_{b} is SH.

Atoms in Formulae (Ill) and (IV), as referenced herein, may be numbered as shown below:

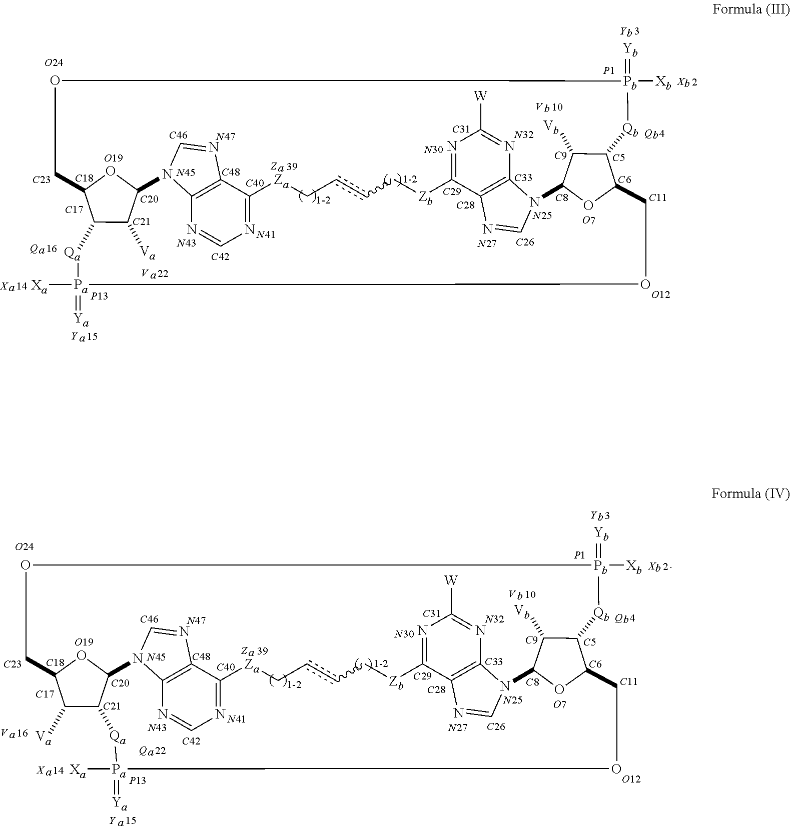

In some embodiments, each of Pₐ and P_{b} is racemic. In some embodiments, Pₐ is racemic and P_{b} is selected from (R)-configuration and (S)-configuration. In some embodiments, Pₐ is selected from (R)-configuration and (S)-configuration and P_{b} is racemic. In some embodiments, each of Pₐ and P_{b} is selected from (R)-configuration and (S)-configuration.

In some embodiments, Pₐ is (R)-configuration and P_{b} is (R)-configuration. In some embodiments, Pₐ is (R)-configuration and P_{b} is (S)-configuration. In some embodiments, Pₐ is (S)-configuration and P_{b} is (R)-configuration. In some embodiments, Pₐ is (S)-configuration and P_{b} is (S)-configuration.

In some embodiments, Qₐ is O and Q_{b} is O. In some embodiments, Qₐ is NH and Q_{b} is O. In some embodiments, Qₐ is O and Q_{b} is NH. In some embodiments, Qₐ is NH and Q_{b} is NH.

In some embodiments, Vₐ is OH and V_{b} is OH. In some embodiments, Vₐ is F and V_{b} is OH. In some embodiments, Vₐ is OH and V_{b} is F. In some embodiments, Vₐ is F and V_{b} is F.

In some embodiments, W is H. In some embodiments, W is NH₂.

In some embodiments, Xₐ is OH and X_{b} is OH. In some embodiments, Xₐ is SH and X_{b} is OH. In some embodiments, Xₐ is OH and X_{b} is SH. In some embodiments, Xₐ is SH and X_{b} is SH.

In some embodiments, Yₐ is O and Y_{b} is O. In some embodiments, Yₐ is S and Y_{b} is O. In some embodiments, Yₐ is O and Y_{b} is S. In some embodiments, Yₐ is S and Y_{b} is S.

In some embodiments, Zₐ is NH and Z_{b} is selected from CH₂, O, and NH. In some embodiments, Zₐ is NH and Z_{b} is CH₂. In some embodiments, Zₐ is NH and Z_{b} is O. In some embodiments, Zₐ is NH and Z_{b} is NH.

In some embodiments, Zₐ is O and Z_{b} is selected from CH₂, O, and NH. In some embodiments, Zₐ is O and Z_{b} is CH₂. In some embodiments, Zₐ is O and Z_{b} is O. In some embodiments, Zₐ is O and Z_{b} is NH.

In some embodiments, Zₐ is CH₂ and Z_{b} is selected from CH₂, O, and NH. In some embodiments, Zₐ is CH₂ and Z_{b} is CH₂. In some embodiments, Zₐ is CH₂ and Z_{b} is O. In some embodiments, Zₐ is CH₂ and Z_{b} is NH.

In some embodiments, [symbol] is a single bond. In some embodiments, [symbol] is a double bond of (E)-configuration. In some embodiments, [symbol] is a double bond of (Z)-configuration. In some embodiments, [symbol] is a triple bond.

In some embodiments, at least one of Xₐ and X_{b} is SH and each of Zₐ and Z_{b} is independently selected from CH₂, O, and NH. In some embodiments, Xₐ is SH and each of Zₐ and Z_{b} is independently selected from CH₂, O, and NH. In some embodiments, X_{b} is SH and each of Zₐ and Z_{b} is independently selected from CH₂, O, and NH. In some embodiments, each of Xₐ and X_{b} is SH and each of Zₐ and Z_{b} is independently selected from CH₂, O, and NH.

In some embodiments, at least one of Zₐ and Z_{b} is NH and Xₐ and X_{b} are selected from OH and SH. In some embodiments, Zₐ is NH and Xₐ and X_{b} are selected from OH and SH. In some embodiments, Z_{b} is NH and Xₐ and X_{b} are selected from OH and SH. In some embodiments, each of Zₐ and Z_{b} is NH and Xₐ and X_{b} are selected from OH and SH.

In some embodiments, the bridge of the drug moiety is an aliphatic group in which at least one CH₂ unit has been replaced by an NH group. In some embodiments, the aliphatic group is fully saturated. In some embodiments, the aliphatic group contains at least one unit of unsaturation. In some embodiments, the bridge is an aliphatic group in which one CH₂ unit has been replaced by an NH group. In some embodiments, the bridge is an aliphatic group in which two CH₂ units have been replaced by an NH group. In some embodiments, the bridge atoms comprise

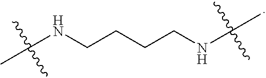

In some embodiments, the bridge atoms comprise

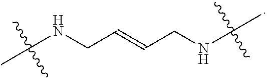 [SMILES: *NC/C=C/CN*]

In some embodiments, the bridge atoms comprise

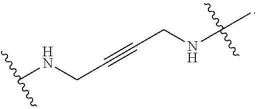 [SMILES: *NCC#CCN*]

In some embodiments, D comprises a compound of Formula (III) and Xₐ is SH. In some embodiments, D comprises a compound of Formula (III) and X_{b} is SH. In some embodiments, D comprises a compound of Formula (IV) and Xₐ is SH. In some embodiments, D comprises a compound of Formula (IV) and X_{b} is SH.

In some embodiments, D comprises a compound of Formula (III) selected from:

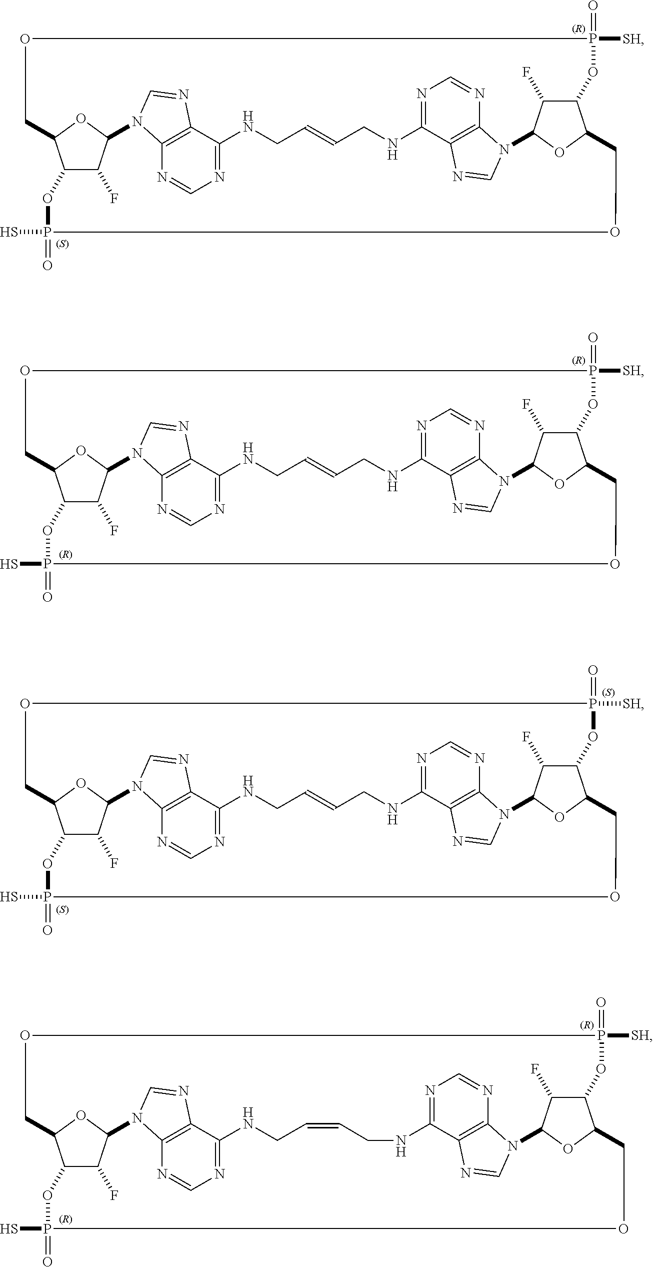

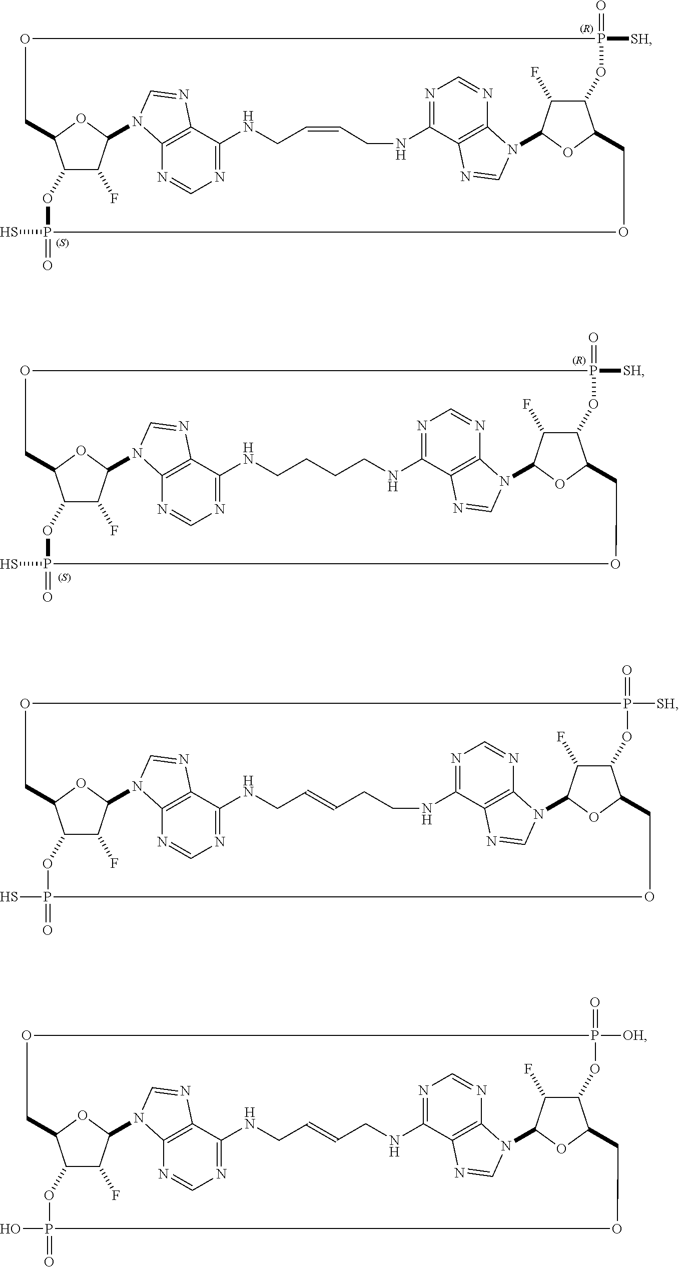

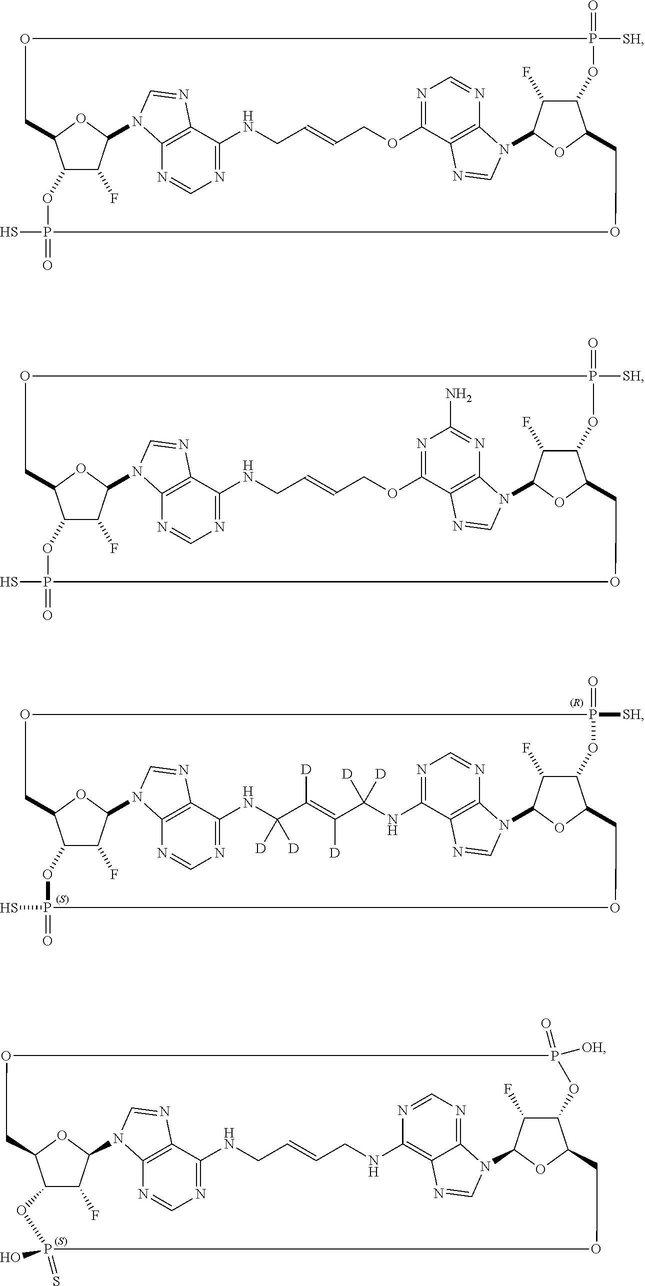

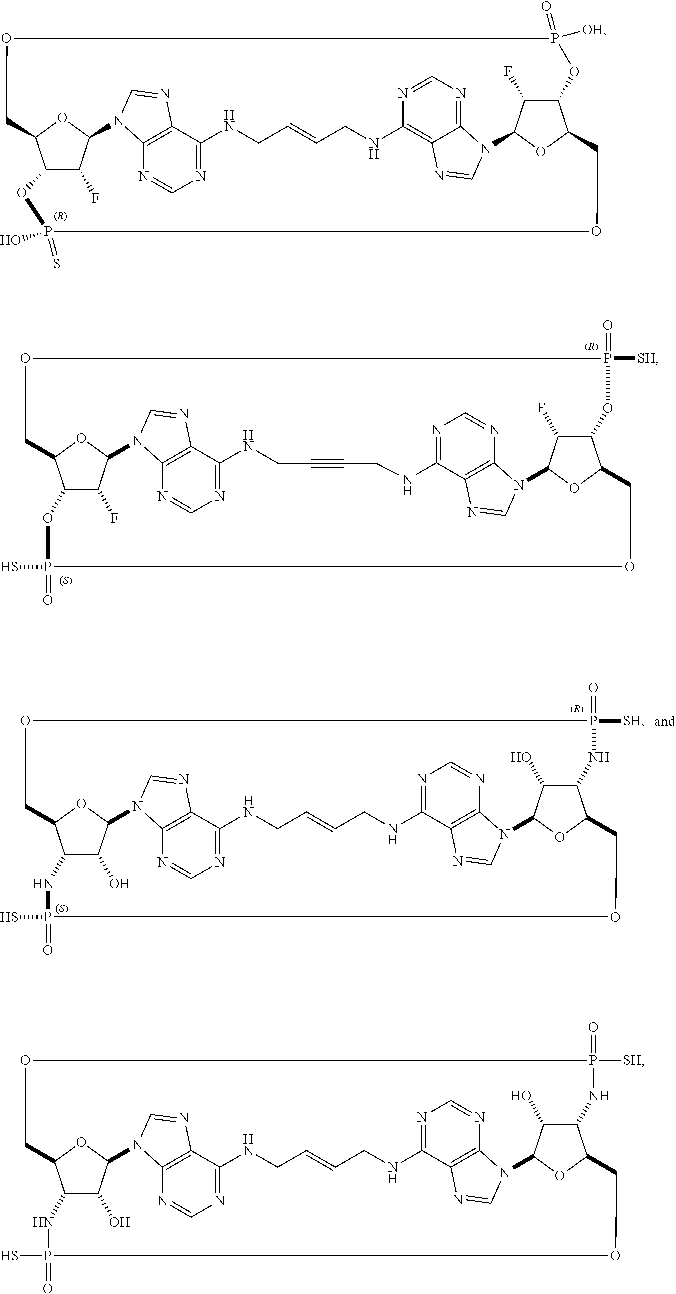

and salts thereof.

In some embodiments, the compound of Formula (III) is selected from:

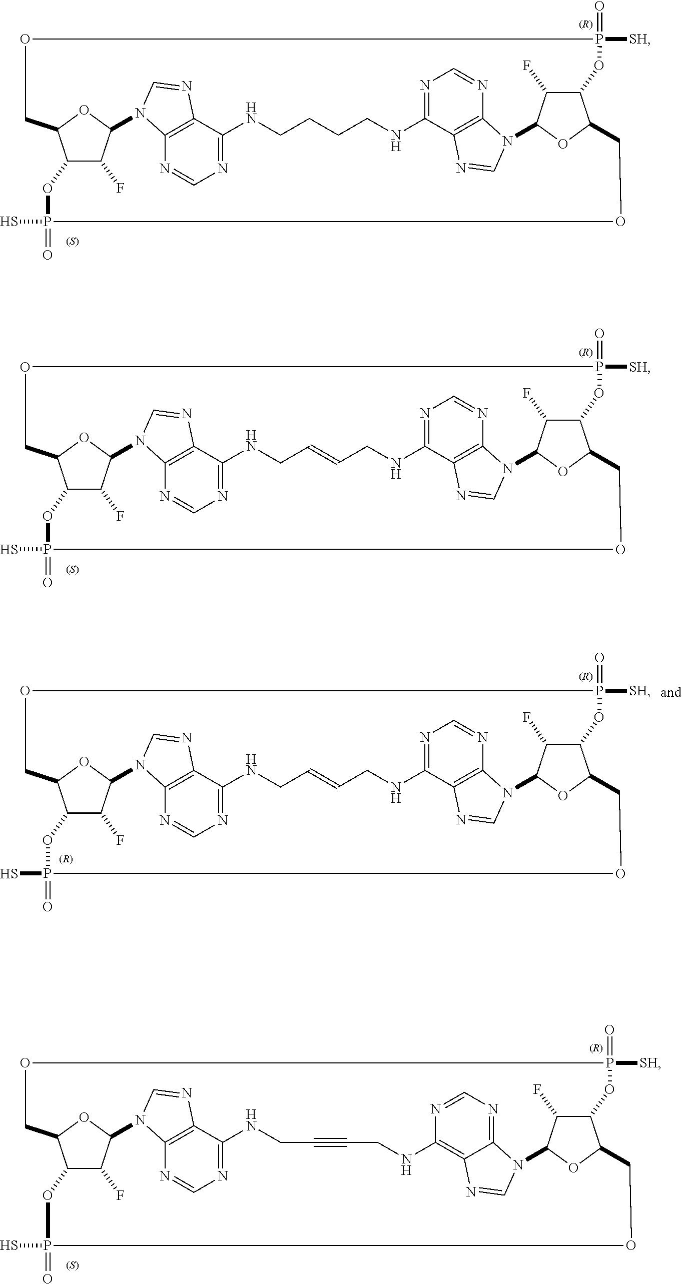

and salts thereof.

In some embodiments, D comprises Compound 1. In some embodiments, D comprises Compound 2.

In some embodiments, D comprises a compound of Formula (IV) selected from:

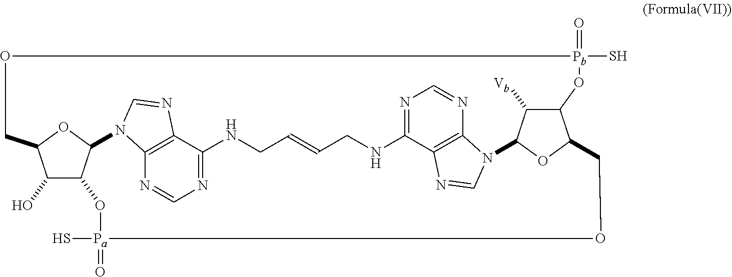

and salts thereof.

In some embodiments, D comprises a compound of Formula (IV) selected from:

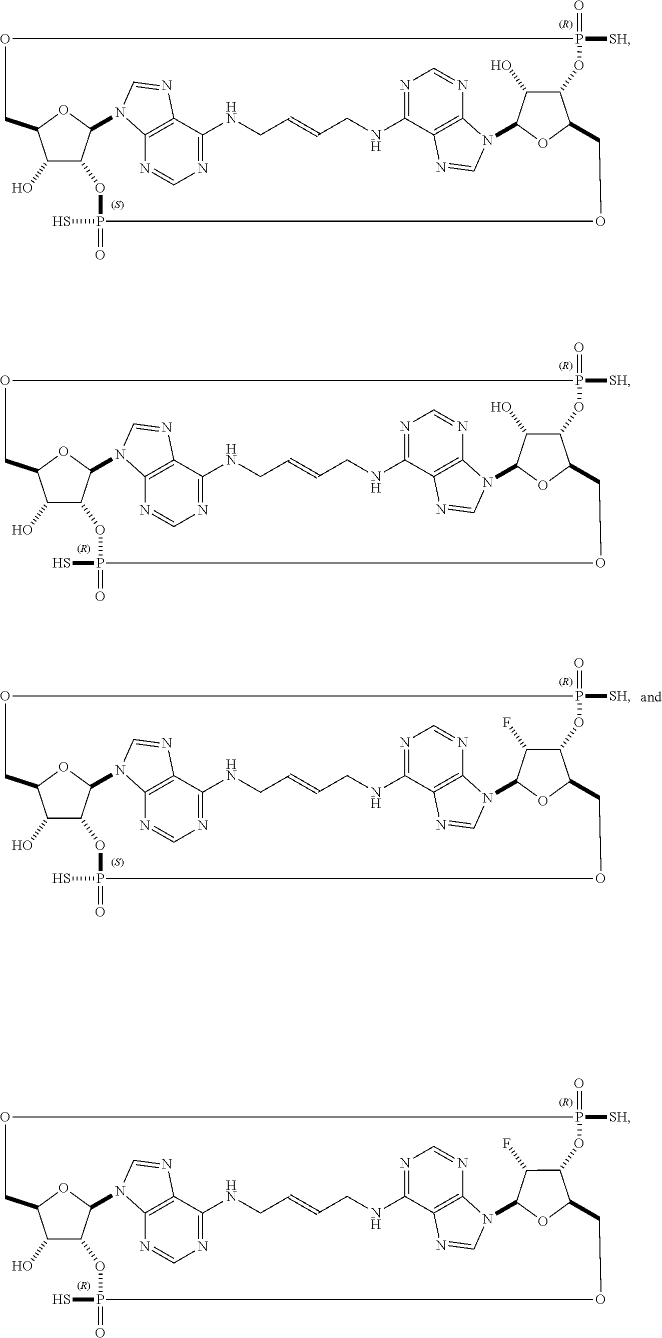

and salts thereof.

In some embodiments, D comprises Compound 1. In some embodiments, D comprises Compound 2.

In some embodiments, D comprises a compound selected from:

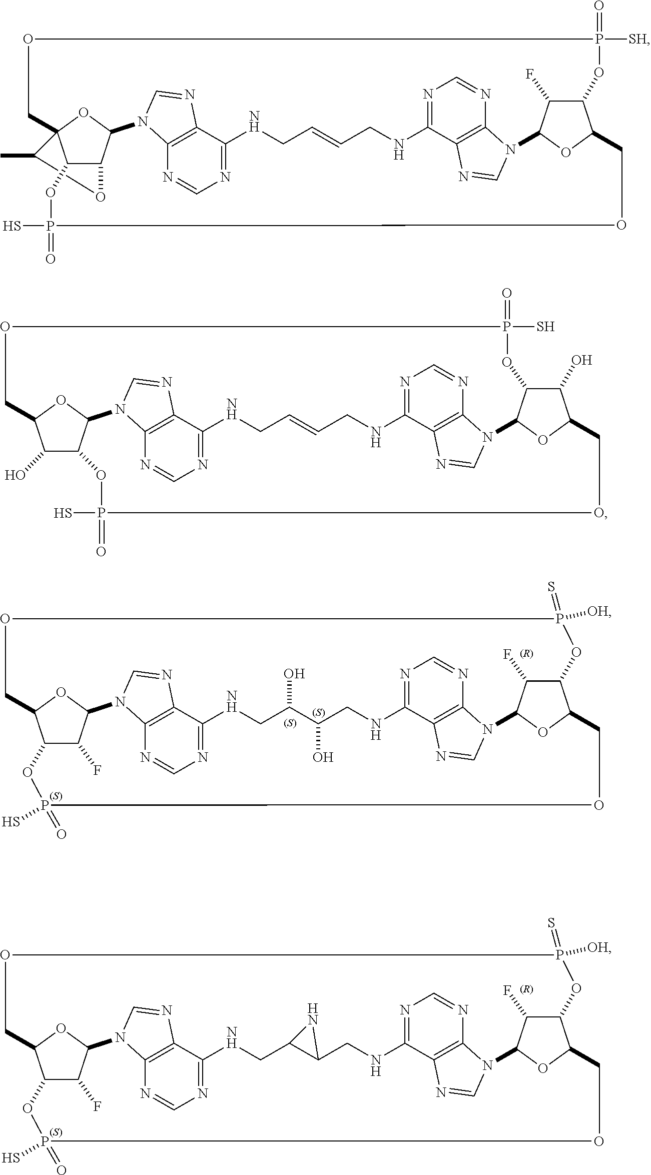

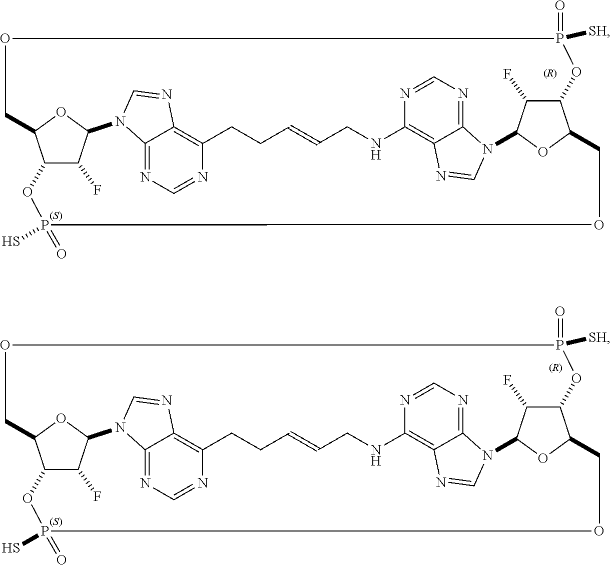

and salts thereof.

In some embodiments, the STING agonist is Compound 1. The structure of Compound 1 is shown below:

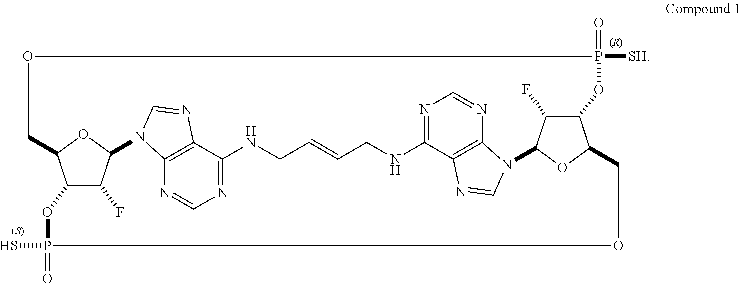

As noted above, the term Compound 1 also encompasses salts of the structure shown above unless context indicates otherwise. In some embodiments, the drug moiety is Compound 1. In some embodiments, a linker, e.g., the linker of an ADC, is attached to Compound 1 via the S-14 sulfur on Compound 1. In some embodiments, a linker, e.g., the linker of an ADC, is attached to Compound 1 via the N-34 nitrogen on Compound 1. In some embodiments, a linker, e.g., the linker of an ADC, is attached to Compound 1 via the N-39 nitrogen on Compound 1. In some embodiments, the linker of the ADC covalently attaches to the S-14 sulfur on Compound 1 via pAB. In some embodiments, the pAB is an analog of pAB as disclosed above. In some embodiments, the linker of the ADC covalently attaches to the N-34 nitrogen on Compound 1 via pABC. In some embodiments, the linker of the ADC covalently attaches to the N-39 nitrogen on Compound 1 via pABC. In some embodiments, the linker of the ADC covalently attaches to the N-34 nitrogen on Compound 1 via a second self immolative unit as disclosed below. In some embodiments, the linker of the ADC covalently attaches to the N-39 nitrogen on Compound 1 via a second self immolative unit as disclosed below.

In some embodiments, the STING agonist is Compound 2. The structure of Compound 2 is shown below:

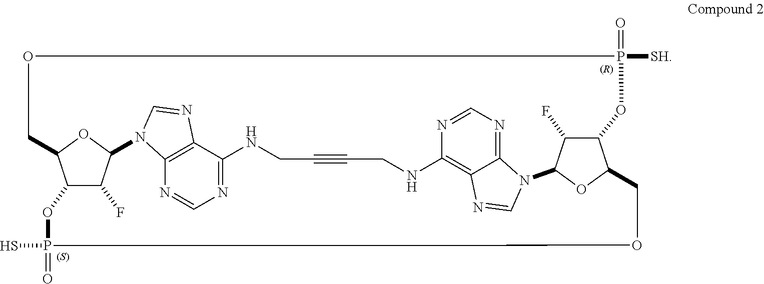

The term Compound 2 also encompasses salts of the structure shown above unless context indicates otherwise. In some embodiments, the drug moiety is Compound 2. In some embodiments, a linker, e.g., the linker of an ADC, is attached to Compound 1 via the S-14 sulfur on Compound 2. In some embodiments, a linker, e.g., the linker of an ADC, is attached to Compound 1 via the N-34 nitrogen on Compound 2. In some embodiments, a linker, e.g., the linker of an ADC, is attached to Compound 1 via the N-39 nitrogen on Compound 2. In some embodiments, the linker of the ADC covalently attaches to the S-14 sulfur on Compound 2 via pAB. In some embodiments, the pAB is an analog of pAB as disclosed above. In some embodiments, the linker of the ADC covalently attaches to the N-34 nitrogen on Compound 2 via pABC. In some embodiments, the linker of the ADC covalently attaches to the N-39 nitrogen on Compound 2 via pABC. In some embodiments, the linker of the ADC covalently attaches to the N-34 nitrogen on Compound 2 via a second self immolative unit as disclosed below. In some embodiments, the linker of the ADC covalently attaches to the N-39 nitrogen on Compound 2 via a second self immolative unit as disclosed below. In some embodiments, the STING agonist is selected from a compound of Table 14, infra.

TABLE 14
Exemplary STING agonist compounds

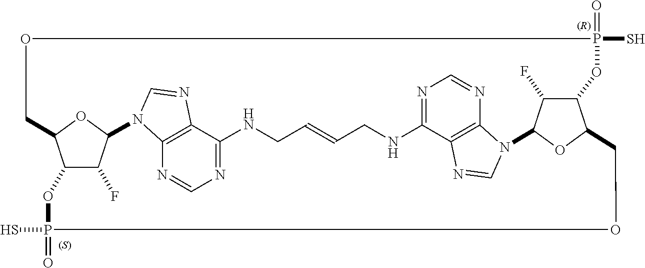

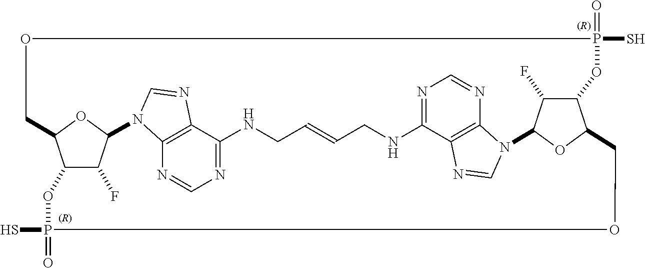

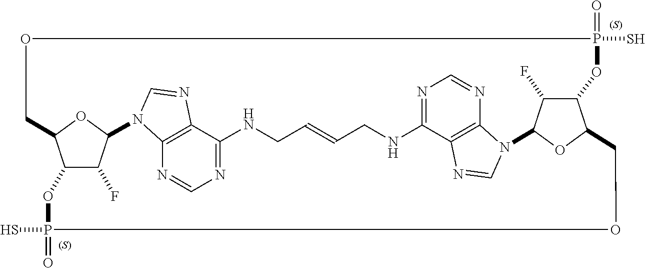

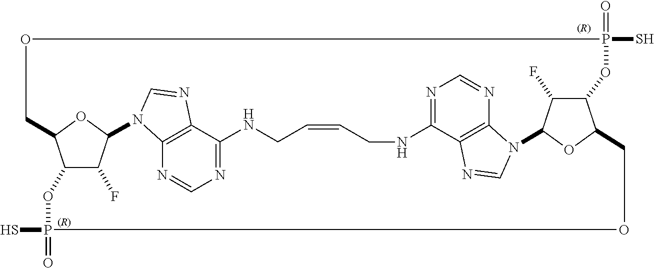

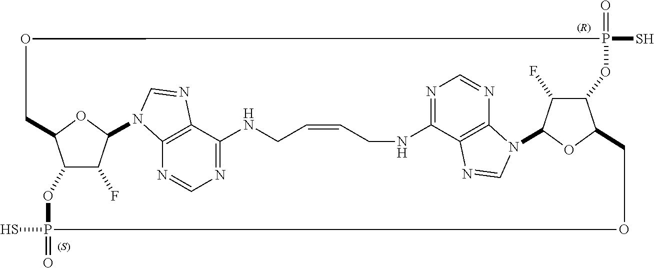

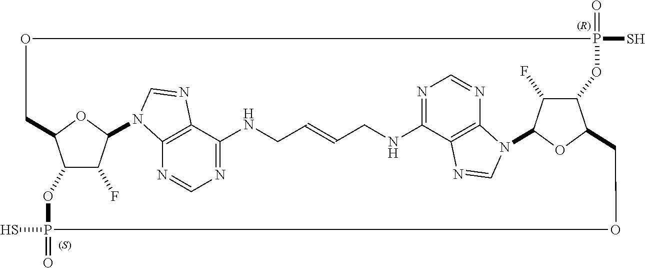

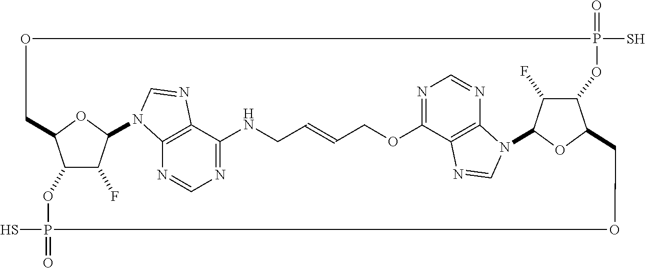

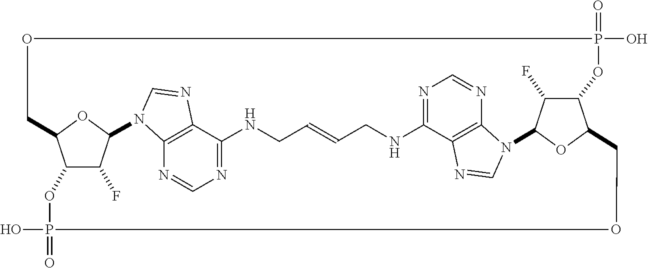

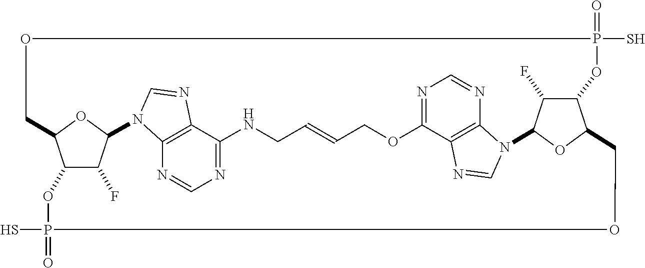

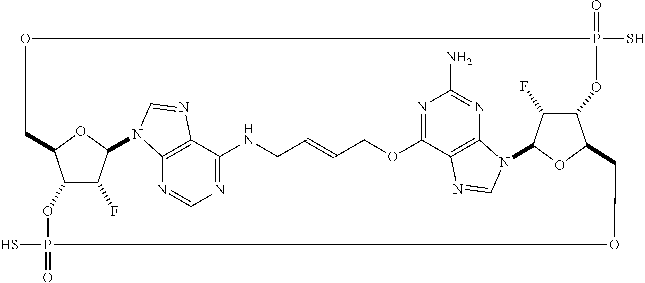

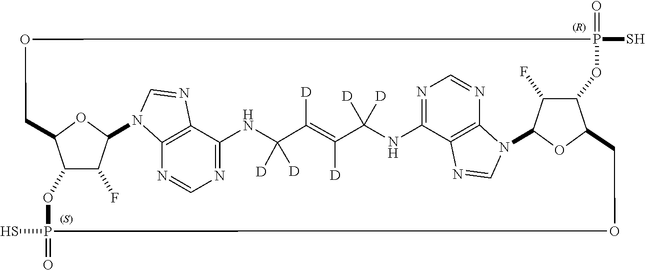

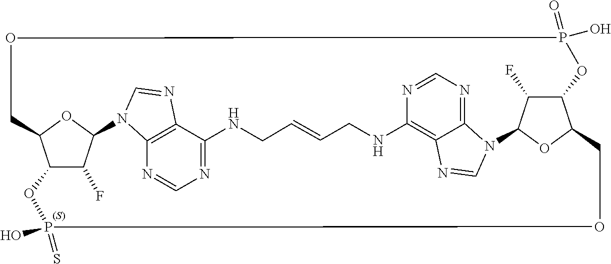

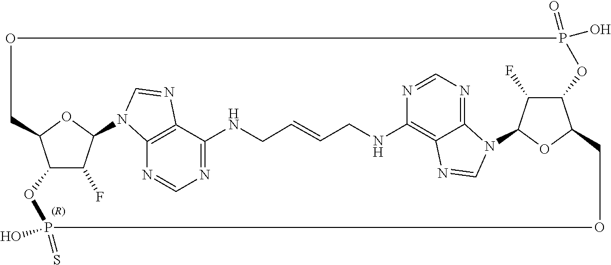

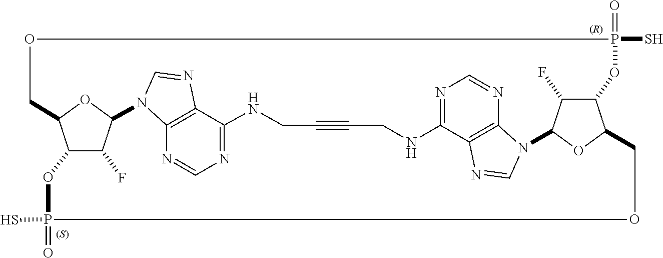

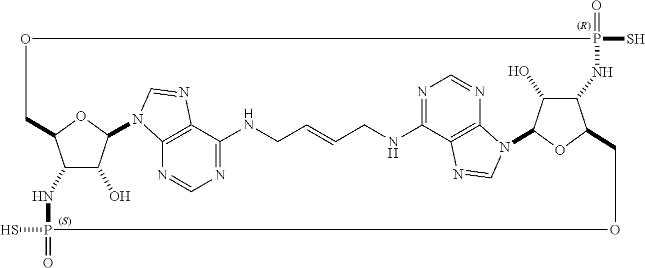

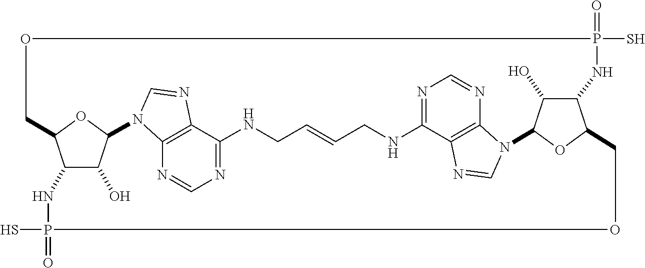

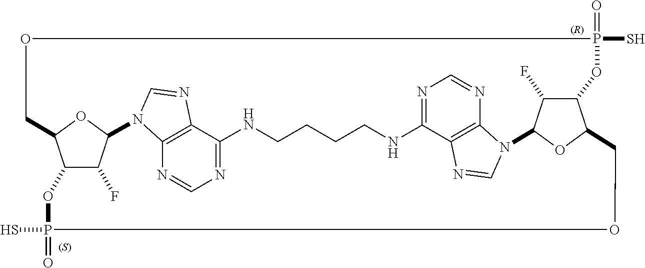

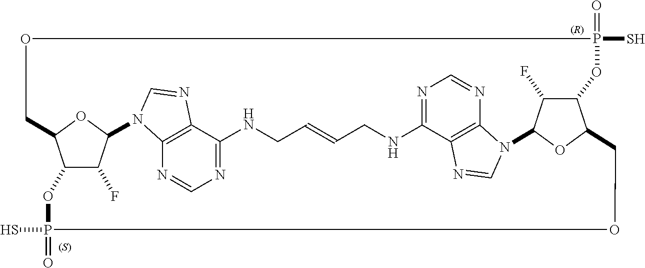

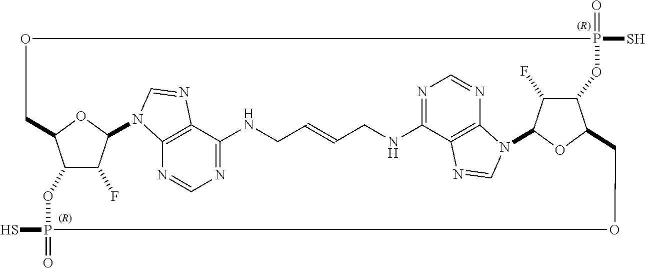

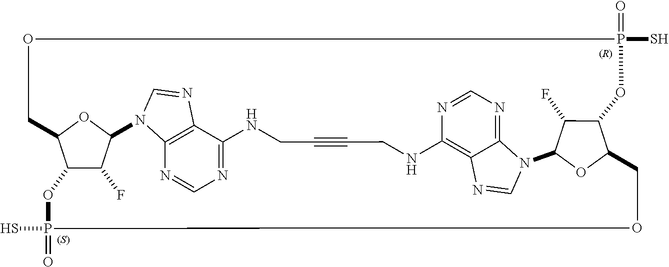

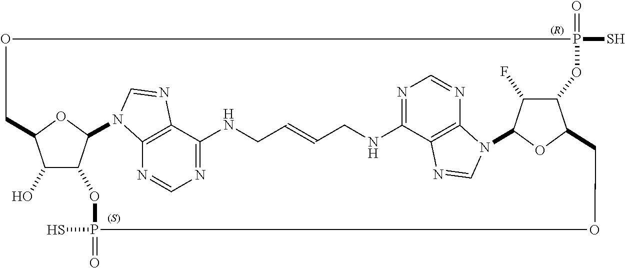

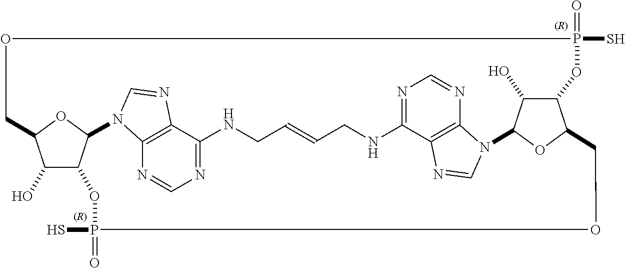

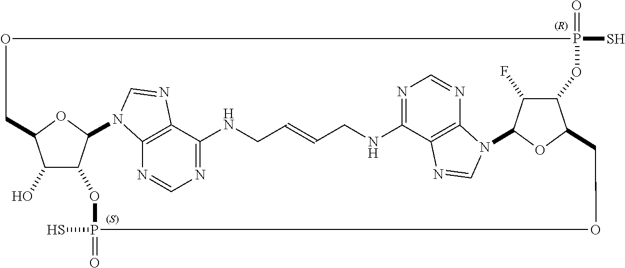

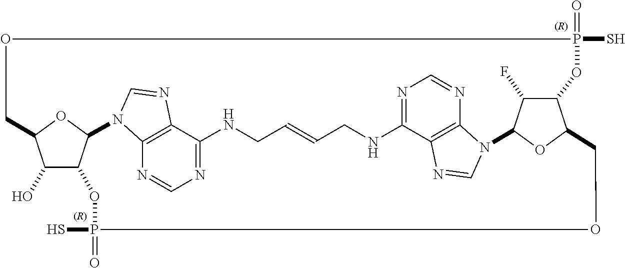

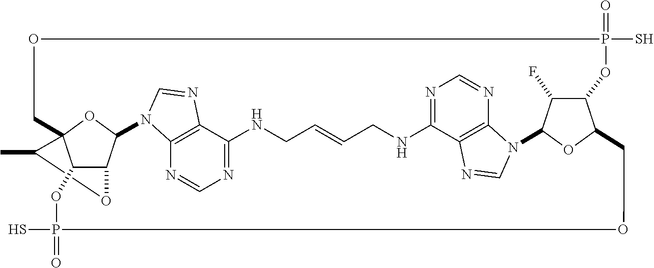

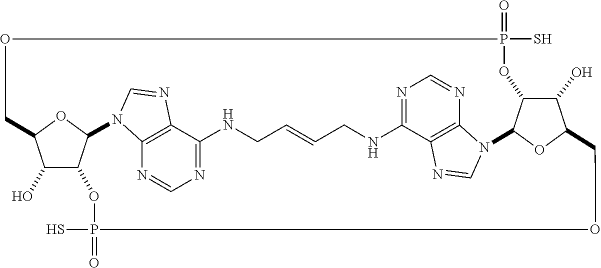

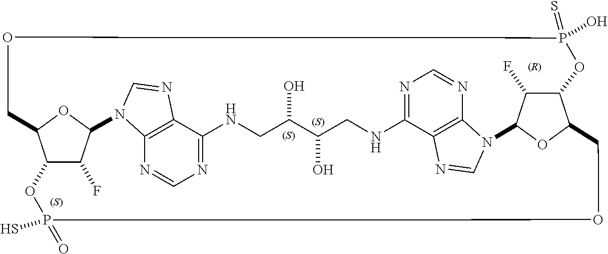

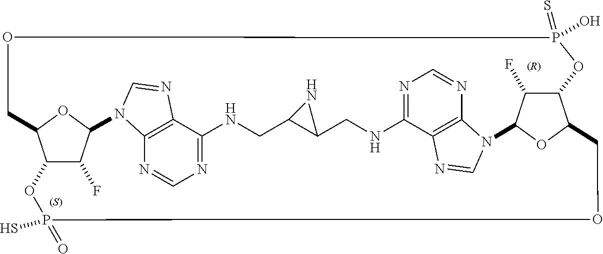

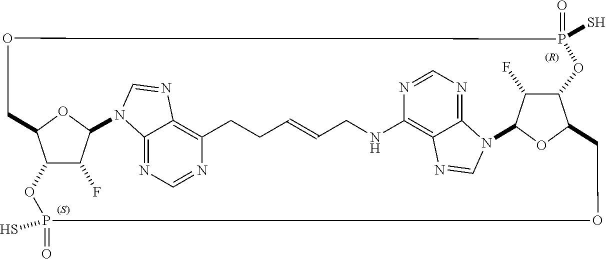

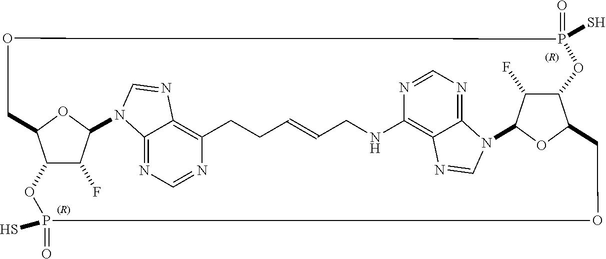

Further disclosed are isomers of the compounds of Table 14, deuterated derivatives of the compounds and isomers; and salts of the compounds, isomers, and deuterated derivatives.

In certain embodiments, an intermediate, such as a precursor of a linker disclosed above, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions, e.g., according to the methods discussed below. Alternatively, the linker or intermediate may first be reacted with the antibody or a derivatized antibody, and then reacted with the drug or derivatized drug.

A number of different reactions are available for covalent attachment of drugs and/or linkers to the antibody moiety. This is often accomplished by reaction of one or more amino acid residues of the antibody molecule, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

### Linker-Drug Conjugates

The present disclosure provides linker-drug conjugates comprising L-D, wherein L is a cleavable linker that covalently attaches to D. The terms “linker-drug conjugate” and “linker-payload conjugate” are used interchangeably herein. The linker-drug conjugates disclosed herein are suitable for conjugation to a variety of antibodies, including anti-PSMA antibodies disclosed herein. In the L-D context, D is a compound that forms a covalent bond with L, which results in the loss of at least one hydrogen radical. In the L-D context, D may be any suitable compound that would benefit from a disclosed linker. In some embodiments, D is selected from any of the compounds disclosed herein. In the L-D context, L may be selected from any linker disclosed herein. D comprises a compound according to one of the following Formulae:

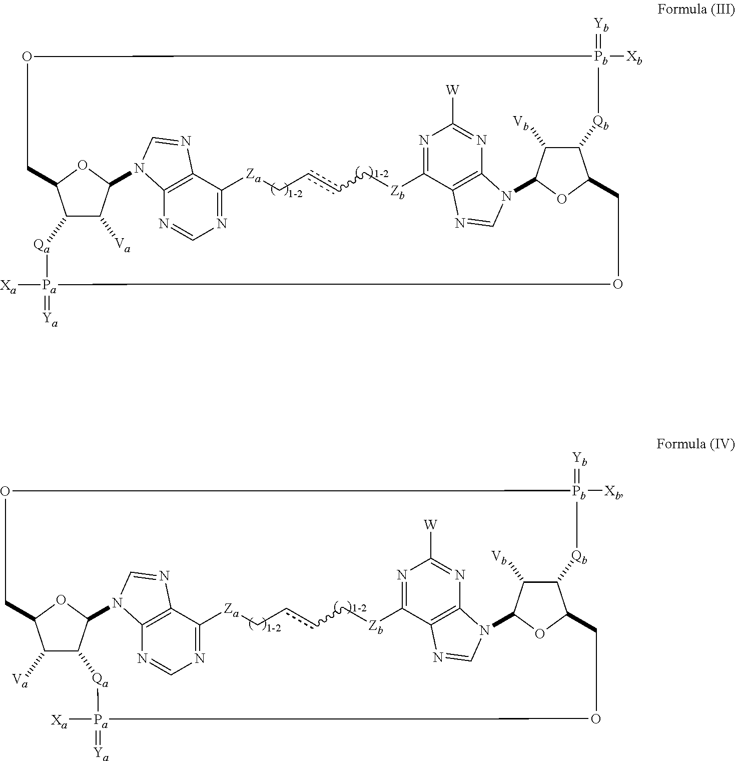

an isomer thereof, a deuterated derivative of the compound or isomer; or a salt of the compound, isomer, or deuterated derivative;
wherein, independently for each occurrence,
each of Pₐ and P_{b}, when not racemic, is independently selected from (R)-configuration and (S)-configuration;
each of Qₐ and Q_{b} is independently selected from NH and O;
each of Vₐ and V_{b} is independently selected from F and OH;
W is selected from H and NH₂;
each of Xₐ and X_{b} is independently selected from OH and SH;
each of Yₐ and Y_{b} is independently selected from O and S;
each Zₐ and Z_{b} is independently selected from CH₂, O, and NH; and
[symbol] means that the bond is selected from a single bond ( [symbol] ), a double bond ( [symbol] ) of (E)- or (Z)-configuration, or a triple bond ( [symbol] );
provided that at least one of Zₐ and Z_{b} is NH or at least one of Xₐ and X_{b} is SH.

In some embodiments, each of Pₐ and P_{b} is racemic. In some embodiments, Pₐ is racemic and P_{b} is selected from (R)-configuration and (S)-configuration. In some embodiments, Pₐ is selected from (R)-configuration and (S)-configuration and P_{b} is racemic. In some embodiments, each of Pₐ and P_{b} is selected from (R)-configuration and (S)-configuration.

In some embodiments, Pₐ is (R)-configuration and P_{b} is (R)-configuration. In some embodiments, Pₐ is (R)-configuration and P_{b} is (S)-configuration. In some embodiments, Pₐ is (S)-configuration and P_{b} is (R)-configuration. In some embodiments, Pₐ is (S)-configuration and P_{b} is (S)-configuration.

In some embodiments, Qₐ is O and Q_{b} is O. In some embodiments, Qₐ is NH and Q_{b} is O. In some embodiments, Qₐ is O and Q_{b} is NH. In some embodiments, Qₐ is NH and Q_{b} is NH.

In some embodiments, Vₐ is OH and V_{b} is OH. In some embodiments, Vₐ is F and V_{b} is OH. In some embodiments, Vₐ is OH and V_{b} is F. In some embodiments, Vₐ is F and V_{b} is F.

In some embodiments, W is H. In some embodiments, W is NH₂.

In some embodiments, Xₐ is OH and X_{b} is OH. In some embodiments, Xₐ is SH and X_{b} is OH. In some embodiments, Xₐ is OH and X_{b} is SH. In some embodiments, Xₐ is SH and X_{b} is SH.

In some embodiments, Yₐ is O and Y_{b} is O. In some embodiments, Yₐ is S and Y_{b} is O. In some embodiments, Yₐ is O and Y_{b} is S. In some embodiments, Yₐ is S and Y_{b} is S.

In some embodiments, Zₐ is NH and Z_{b} is selected from CH₂, O, and NH. In some embodiments, Zₐ is NH and Z_{b} is CH₂. In some embodiments, Zₐ is NH and Z_{b} is O. In some embodiments, Zₐ is NH and Z_{b} is NH.

In some embodiments, Zₐ is O and Z_{b} is selected from CH₂, O, and NH. In some embodiments, Zₐ is O and Z_{b} is CH₂. In some embodiments, Zₐ is O and Z_{b} is O. In some embodiments, Zₐ is O and Z_{b} is NH.

In some embodiments, Zₐ is CH₂ and Z_{b} is selected from CH₂, O, and NH. In some embodiments, Zₐ is CH₂ and Z_{b} is CH₂. In some embodiments, Zₐ is CH₂ and Z_{b} is O. In some embodiments, Zₐ is CH₂ and Z_{b} is NH.

In some embodiments, [symbol] is a single bond. In some embodiments, [symbol] is a double bond of (E)-configuration. In some embodiments, [symbol] is a double bond of (Z)-configuration. In some embodiments, [symbol] is a triple bond.

In some embodiments, at least one of Xₐ and X_{b} is SH and each of Zₐ and Z_{b} is independently selected from CH₂, O, and NH. In some embodiments, Xₐ is SH and each of Zₐ and Z_{b} is independently selected from CH₂, O, and NH. In some embodiments, X_{b} is SH and each of Zₐ and Z_{b} is independently selected from CH₂, O, and NH. In some embodiments, each of Xₐ and X_{b} is SH and each of Zₐ and Z_{b} is independently selected from CH₂, O, and NH.

In some embodiments, at least one of Zₐ and Z_{b} is NH and Xₐ and X_{b} are selected from OH and SH. In some embodiments, Zₐ is NH and Xₐ and X_{b} are selected from OH and SH. In some embodiments, Z_{b} is NH and Xₐ and X_{b} are selected from OH and SH. In some embodiments, each of Zₐ and Z_{b} is NH and Xₐ and X_{b} are selected from OH and SH.

In some embodiments, D comprises a compound of Formula (III) and Xₐ is SH. In some embodiments, D comprises a compound of Formula (III) and X_{b} is SH. In some embodiments, D comprises a compound of Formula (IV) and Xₐ is SH. In some embodiments, D comprises a compound of Formula (IV) and X_{b} is SH.

In some embodiments, the bridge of the linker-drug conjugate is an aliphatic group in which at least one CH₂ unit has been replaced by an NH group. In some embodiments, the aliphatic group is fully saturated. In some embodiments, the aliphatic group contains at least one unit of unsaturation.

In some embodiments, the bridge is an aliphatic group in which one CH₂ unit has been replaced by an NH group. In some embodiments, the bridge is an aliphatic group in which two CH₂ units have been replaced by an NH group. In some embodiments, the bridge atoms comprise

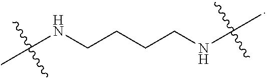 [SMILES: *NCCCCN*]

In some embodiments, the bridge atoms comprise

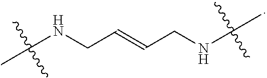 [SMILES: *NC/C=C/CN*]

In some embodiments, the bridge atoms comprise

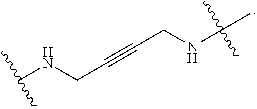 [SMILES: *NCC#CCN*]

In some embodiments, L is attached to D via a sulfur atom. In some embodiments, L is attached to D at the S-2 sulfur or the S-14 sulfur. In some embodiments, L is attached to D at the S-2 sulfur. In some embodiments, L is attached to D at the S-14 sulfur.

In some embodiments, D comprises a compound of Formula (III) and Zₐ is NH. In some embodiments, D comprises a compound of Formula (III) and Z_{b} is NH. In some embodiments, D comprises a compound of Formula (IV) and Zₐ is NH. In some embodiments, D comprises a compound of Formula (IV) and Z_{b} is NH.

In some embodiments, L is attached to D via a bridge nitrogen atom. In some embodiments, L is attached to D at the N-34 nitrogen or the N-39 nitrogen. In some embodiments, L is attached to D at the N-34 nitrogen. In some embodiments, L is attached to D at the N-39 nitrogen.

In some embodiments, D comprises a compound of Formula (III). Exemplary compounds of Formula (III) are shown below. In some embodiments, D comprises a compound of Formula (III) selected from:

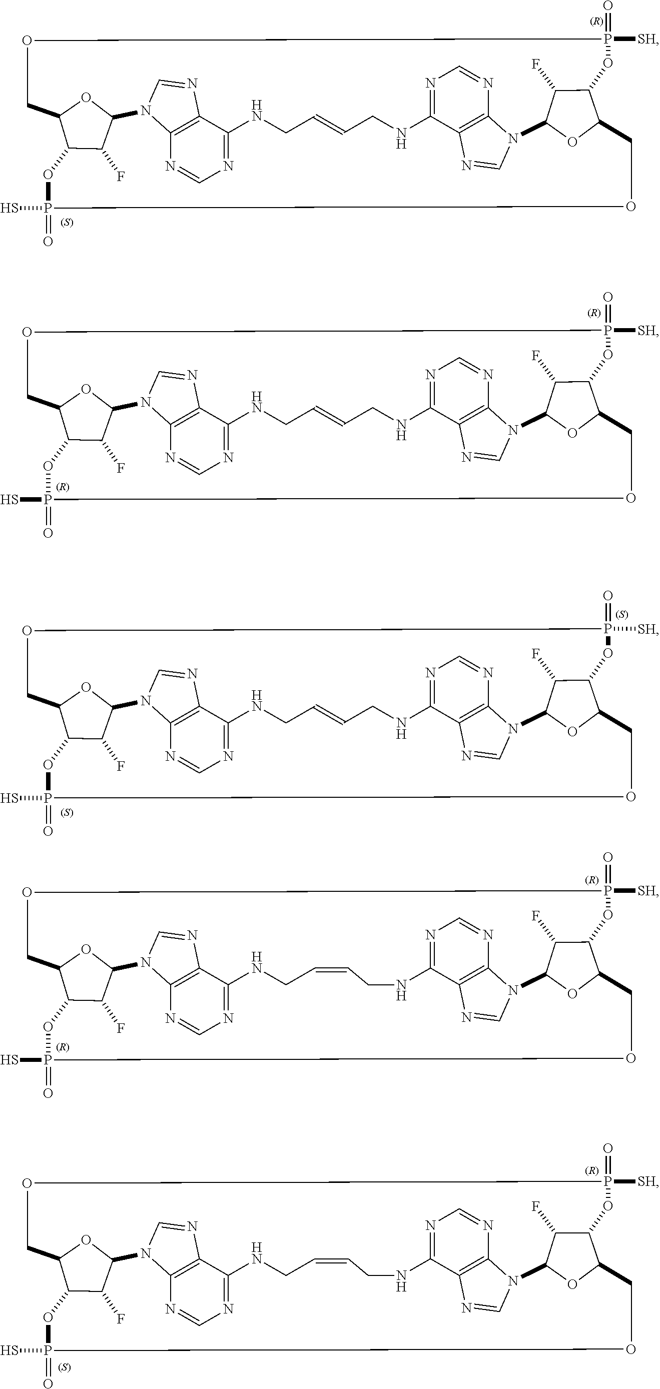

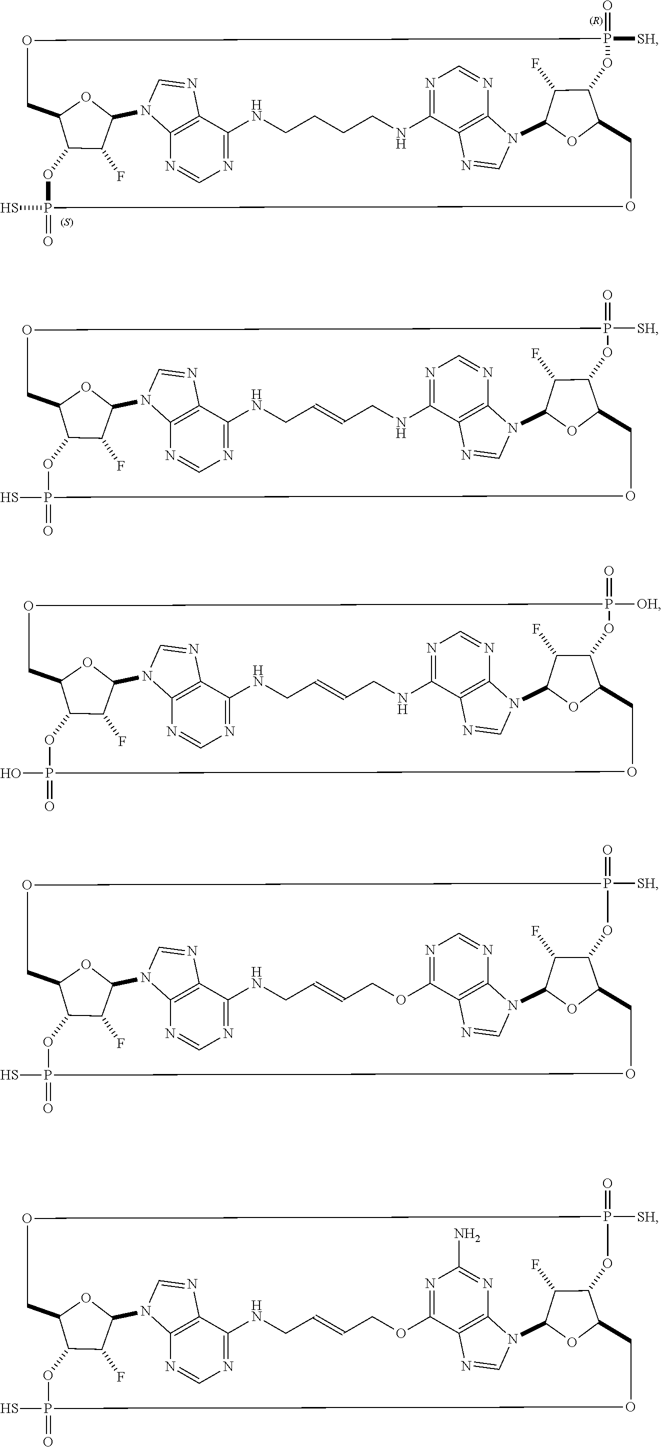

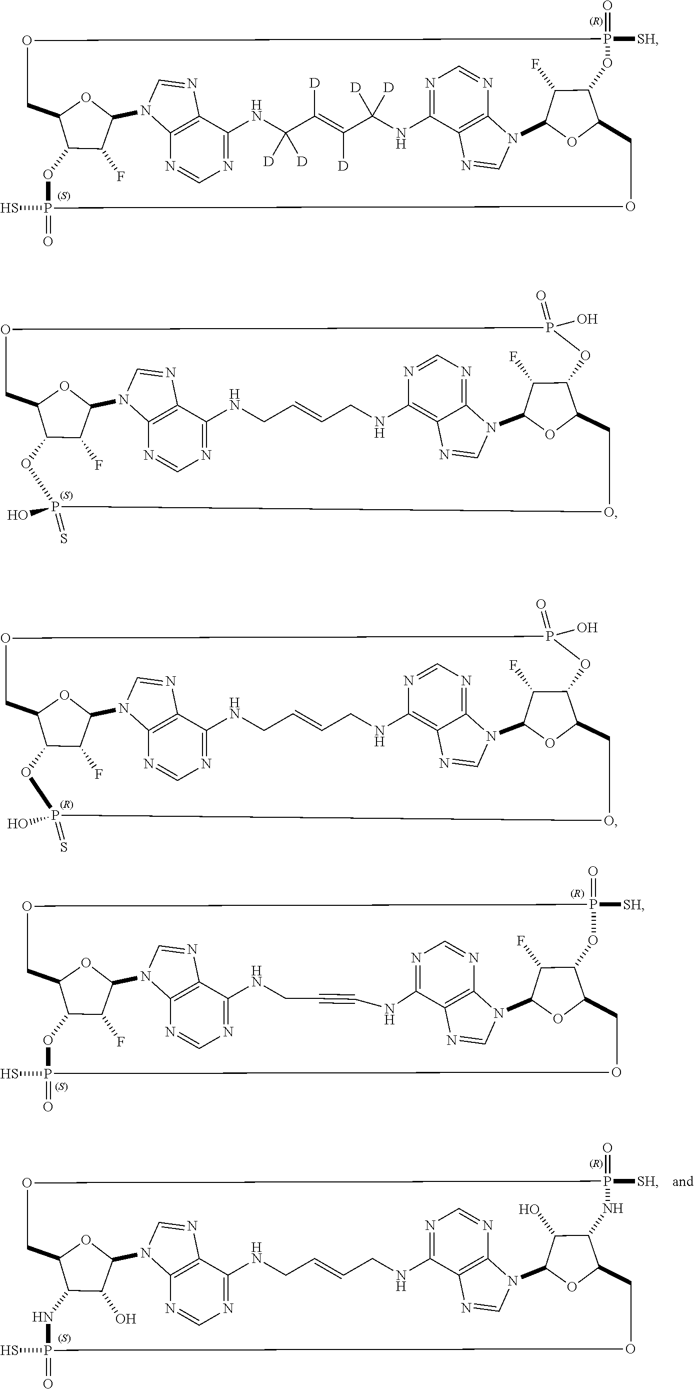

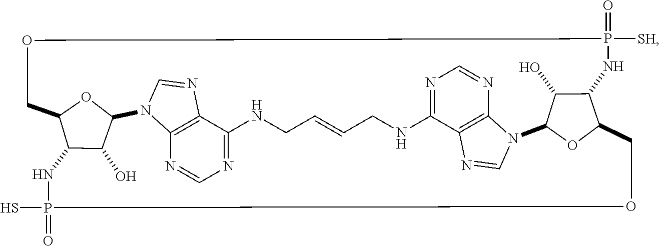

and salts thereof.

In some embodiments, the compound of Formula (III) is selected from:

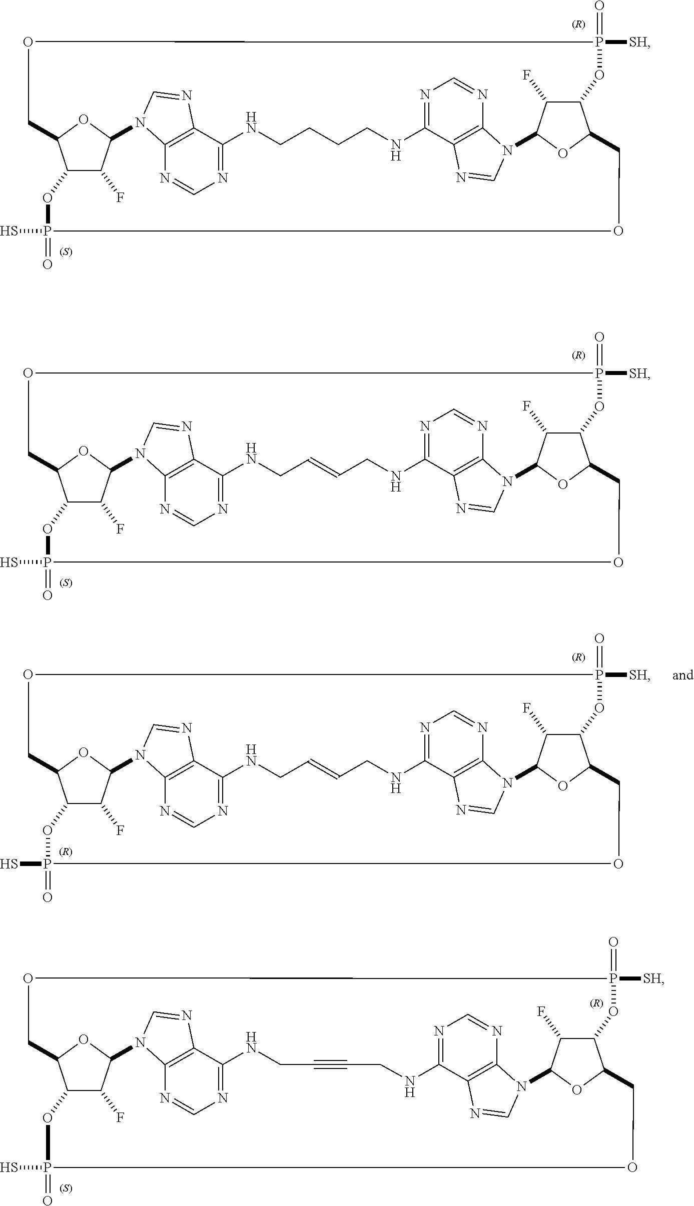

and salts thereof.

In some embodiments, D comprises Compound 1. In some embodiments, D comprises Compound 2.

In some embodiments, D comprises a compound of Formula (IV) selected from:

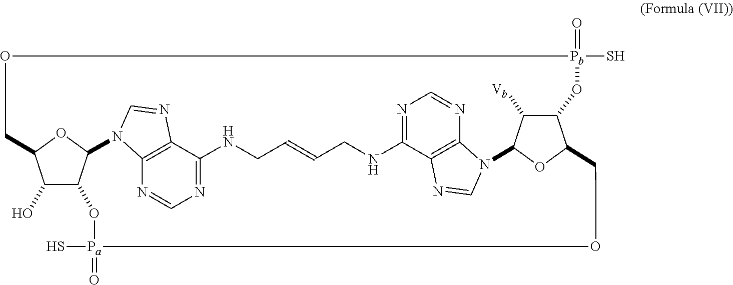

and salts thereof.

In some embodiments, D comprises a compound of Formula (IV) selected from:

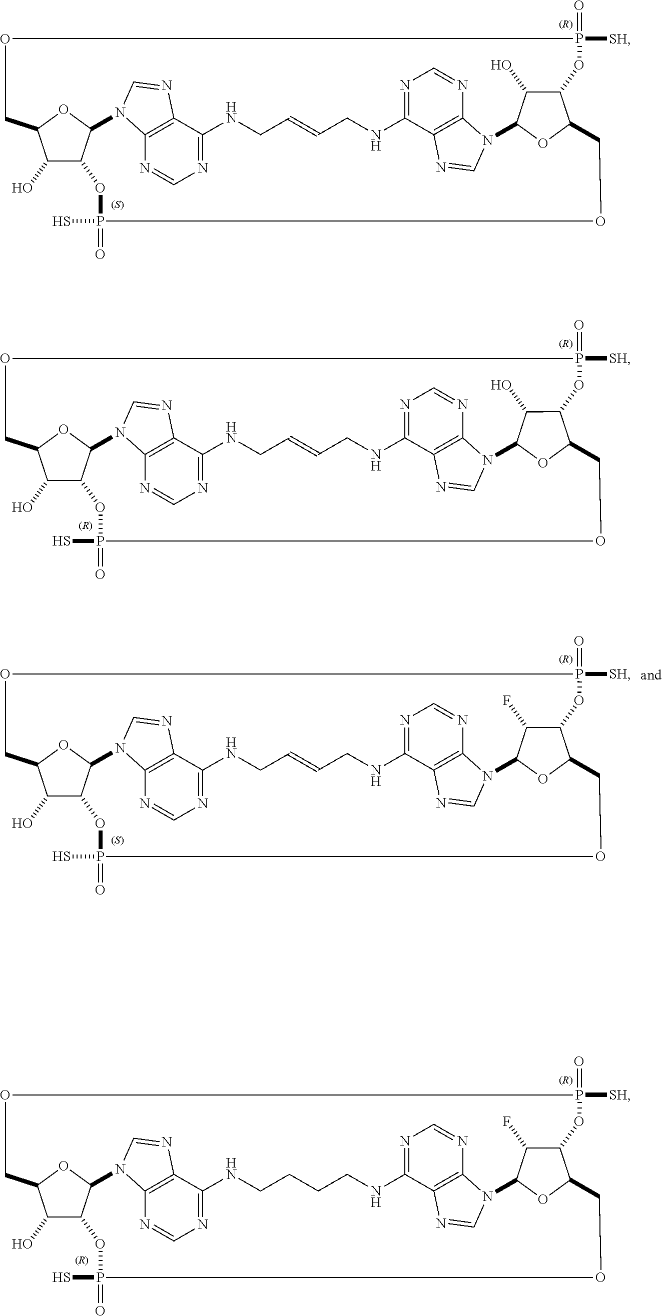

and salts thereof.

In some embodiments, Xₐ or X_{b} is SH and L is attached to D via a sulfur atom at the S-2 sulfur or the S-14 sulfur. In some embodiments, Zₐ or Z_{b} is NH and L is attached to D via a nitrogen atom at the N-34 nitrogen or the N-39 nitrogen.

In some embodiments, D comprises a compound of Formula (III), Xₐ is SH, and L is attached to D at the S-2 sulfur. In some embodiments, D comprises a compound of Formula (III), X_{b} is SH, and L is attached to D at the S-14 sulfur. In some embodiments, D comprises a compound of Formula (III), Zₐ is NH, and L is attached to D at the N-34 nitrogen. In some embodiments, D comprises a compound of Formula (III), Z_{b} is NH, and L is attached to D at the N-39 nitrogen.

In some embodiments, D comprises a compound of Formula (IV) and L is attached to D at the S-2 sulfur. In some embodiments, D comprises a compound of Formula (IV) and L is attached to D at the S-14 sulfur. In some embodiments, D comprises a compound of Formula (IV) and L is attached to D at the N-34 nitrogen. In some embodiments, D comprises a compound of Formula (IV) and L is attached to D at the N-39 nitrogen.

In some embodiments, D comprises Compound 1. In some embodiments, D comprises Compound 2.

The present disclosure provides linker-payload conjugates comprising L-D, wherein L is a cleavable linker that covalently attaches to D, wherein D comprises a compound selected from:

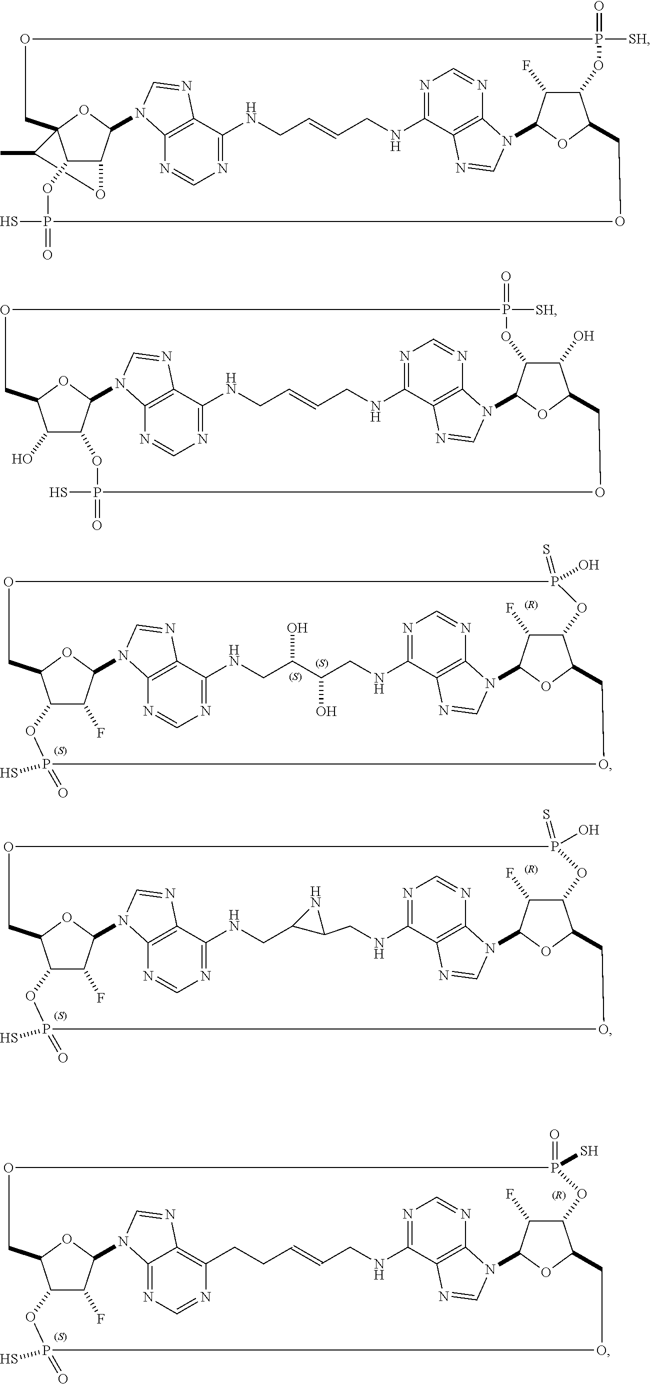

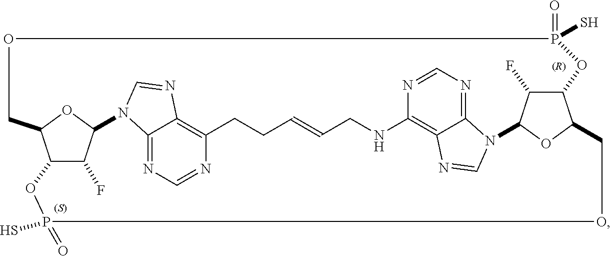

and salts thereof.

In some embodiments, L is attached to D via a sulfur atom at the S-2 sulfur or the 5-14 sulfur. In some embodiments, L is attached to D at the S-2 sulfur. In some embodiments, L is attached to D at the 5-14 sulfur.

In some embodiments, L is attached to D via a nitrogen atom at the N-34 nitrogen or the N-39 nitrogen. In some embodiments, L is attached to D at the N-34 nitrogen. In some embodiments, L is attached to D at the N-39 nitrogen.

In some embodiments of linker-payload conjugates comprising L-D, L is any linker disclosed herein. In some embodiments of linker-payload conjugates comprising L-D, D is any drug moiety disclosed herein.

In some embodiments of linker-payload conjugates comprising L-D, wherein L is a cleavable linker that covalently attaches to D, the cleavable linker comprises a cleavable peptide moiety. In some embodiments, the cleavable peptide moiety is cleavable by a protease. In some embodiments, the protease is legumain or cathepsin. In some embodiments, the cleavable peptide moiety comprises an amino acid unit. In some embodiments, the amino acid unit comprises Val-Ala, Val-Cit, Val-Lys, Ala-Ala-Asn, Ala-(NMe)Ala-Asn, Asn, Gly-Gly-Phe-Gly (SEQ ID NO: 141), or Gly-Val-Ala. In some embodiments, the amino acid unit comprises Val-Ala. In some embodiments, the amino acid unit comprises Val-Cit.

In some embodiments, the linker-payload conjugate comprises Val-Ala, and D is selected from a compound of Table 14. In some embodiments, the linker-payload conjugate comprises Val-Cit, and D is selected from a compound of Table 14.

In some embodiments, the linker-payload conjugate comprises Formula (II), and D is selected from a compound of Table 14.

In some embodiments, the linker-payload conjugate comprises Formula (II)-Val-Ala, and D is selected from a compound of Table 14. In some embodiments, the linker-payload conjugate comprises Formula (II)-Val-Cit, and D is selected from a compound of Table 14.

In some embodiments, the linker-drug conjugate comprises MC-Val-Cit-pABC-MEC-Compound 1. In some embodiments, the linker-drug conjugate comprises MC-Val-Ala-pABC-MEC-Compound 1 (e.g., LP1 or LP2). In some embodiments, the linker-drug conjugate comprises MC-Val-Cit-pABC-Unit 8-Compound 1. In some embodiments, the linker-drug conjugate comprises MC-Val-Ala-pABC-Unit 8-Compound 1 (e.g., LP16). In some embodiments, the linker-drug conjugate comprises MC-Val-Cit-pABC-Unit 9-Compound 1. In some embodiments, the linker-drug conjugate comprises MC-Val-Ala-pABC-Unit 9-Compound 1 (e.g., LP20). In some embodiments, the linker-drug conjugate comprises MC-Val-Cit-pABC-Unit 11-Compound 1. In some embodiments, the linker-drug conjugate comprises MC-Val-Ala-pABC-Unit 11-Compound 1 (e.g., LP28).

In some embodiments, the linker-drug conjugate comprises Mal-Formula (II)-Val-Cit-pABC-MEC-Compound 1. In some embodiments, the linker-drug conjugate comprises Mal-Formula (II)-Val-Cit-pABC-Unit 8-Compound 1. In some embodiments, the linker-drug conjugate comprises Mal-Formula (II)-Val-Cit-pABC-Unit 9-Compound 1. In some embodiments, the linker-drug conjugate comprises Mal-Formula (II)-Val-Cit-pABC-Unit 11-Compound 1. In some embodiments, the linker-drug conjugate comprises Mal-Formula (II)-Val-Ala-pABC-MEC-Compound 1. In some embodiments, the linker-drug conjugate comprises Mal-Formula (II)-Val-Ala-pABC-Unit 8-Compound 1. In some embodiments, the linker-drug conjugate comprises Mal-Formula (II)-Val-Ala-pABC-Unit 9-Compound 1. In some embodiments, the linker-drug conjugate comprises Mal-Formula (II)-Val-Ala-pABC-Unit 11-Compound 1.

In some embodiments, the linker-drug conjugate comprises Mal-Formula (II)-Val-Cit-pAB-Unit 9-Compound 1. In some embodiments, the linker-drug conjugate comprises Mal-Formula (II)-Val-Ala-pAB-Unit 9-Compound 1. In some embodiments, the linker-drug conjugate comprises LP25.

In some embodiments, the linker-drug conjugate comprises Mal-Formula (II)-Val-Cit-pAB-Unit 11-Compound 1. In some embodiments, the linker-drug conjugate comprises Mal-Formula (II)-Val-Ala-pAB-Unit 11-Compound 1. In some embodiments, the linker-drug conjugate comprises LP26.

Exemplary linker-drug conjugates of the invention are disclosed in Table 15 and 16, infra. In various embodiments, the linker-drug conjugate is selected from the linker-drug conjugates shown in Tables 15 and 16.

TABLE 15
Exemplary S-attached Linker-Drug Conjugates

| | |
|---|---|
| LP3 | 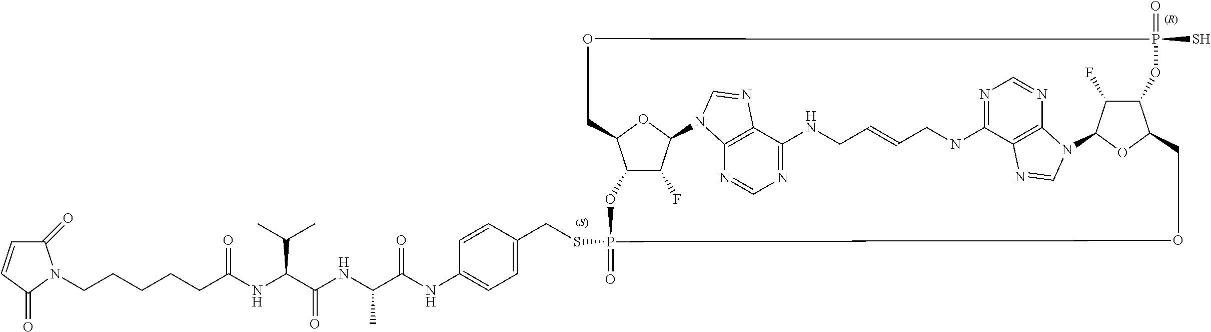 |
| LP33 | 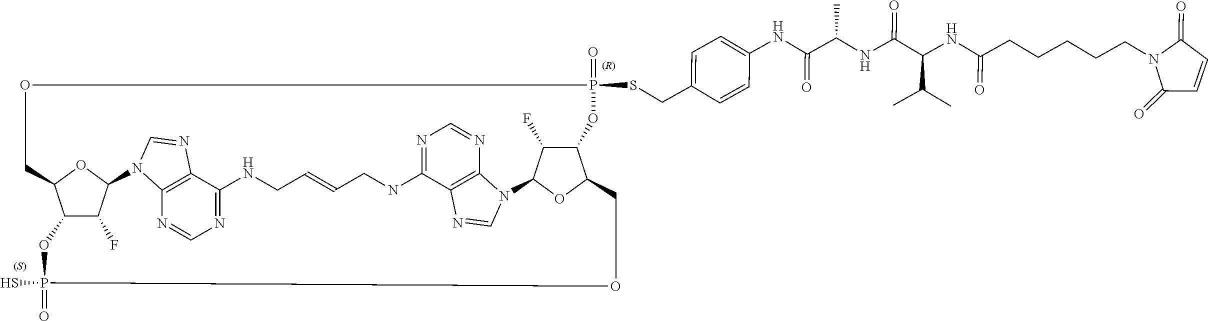 |
| LP34 | 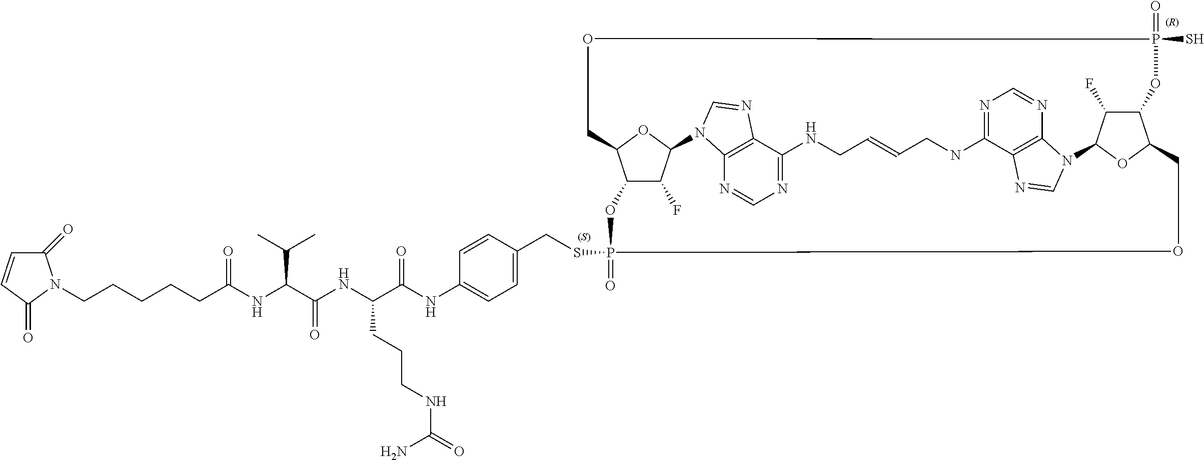 |
| LP35 | 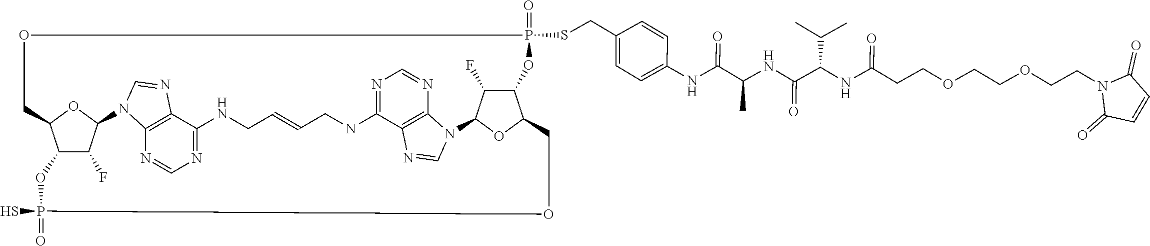 [SMILES: CC(C)[C@H](NC(=O)CCOCCOCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)cc1] |
| LP36 | 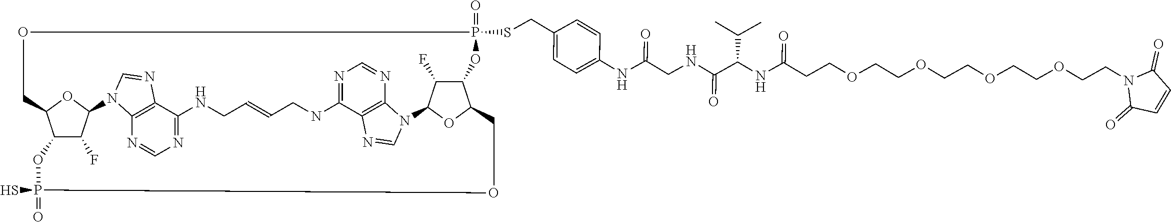 |
| LP37 | 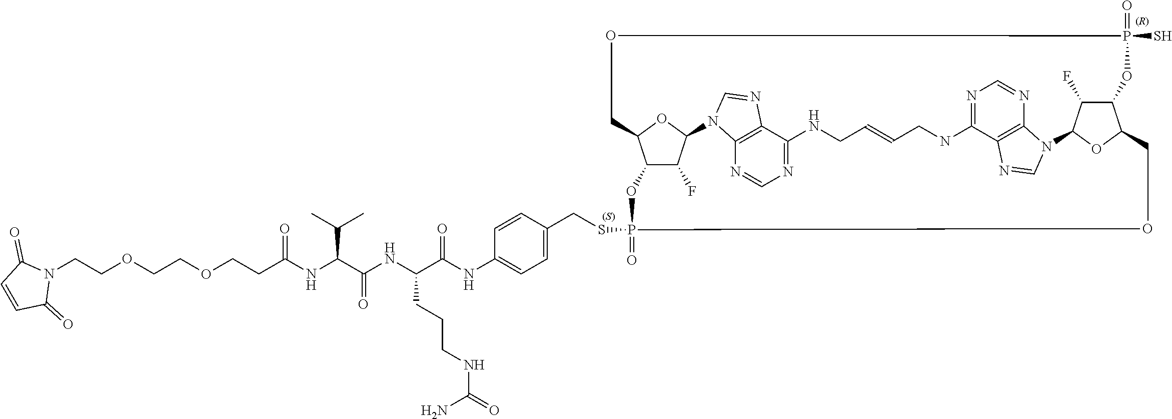 |
| LP38 | 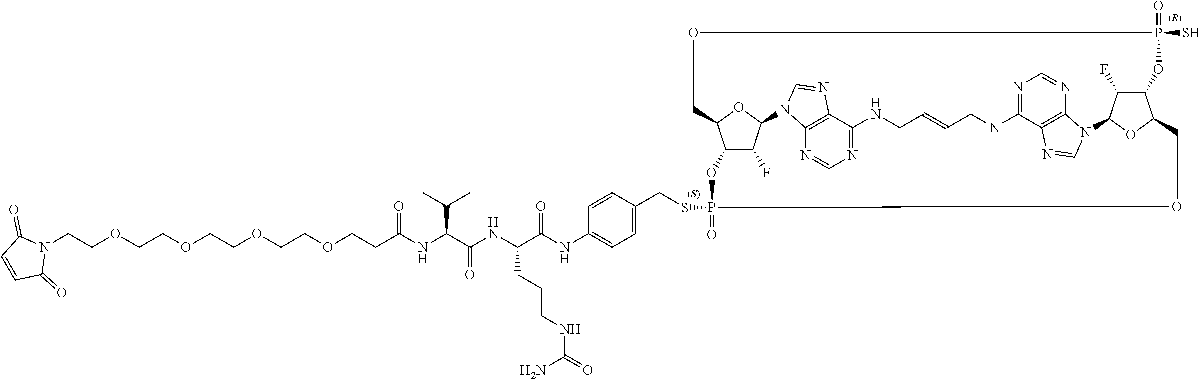 |
| LP39 | 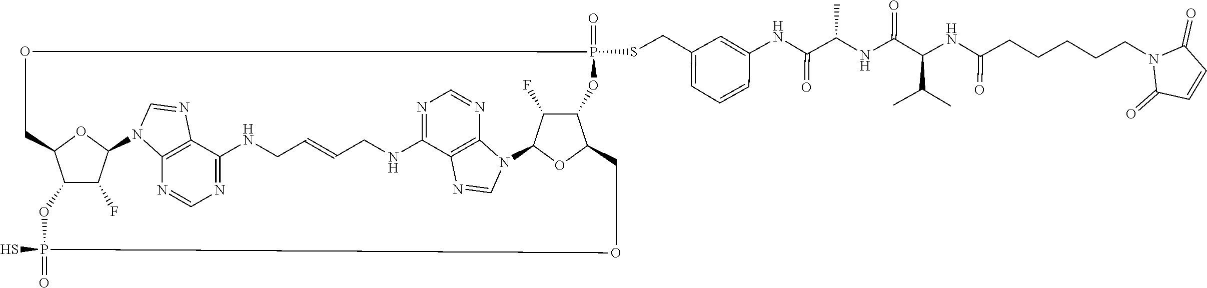 [SMILES: CC(C)[C@H](NC(=O)CCCCCN1C(=O)C=CC1=O)C(=O)N[C@@H](C)C(=O)Nc1cccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)c1] |
| LP40 | 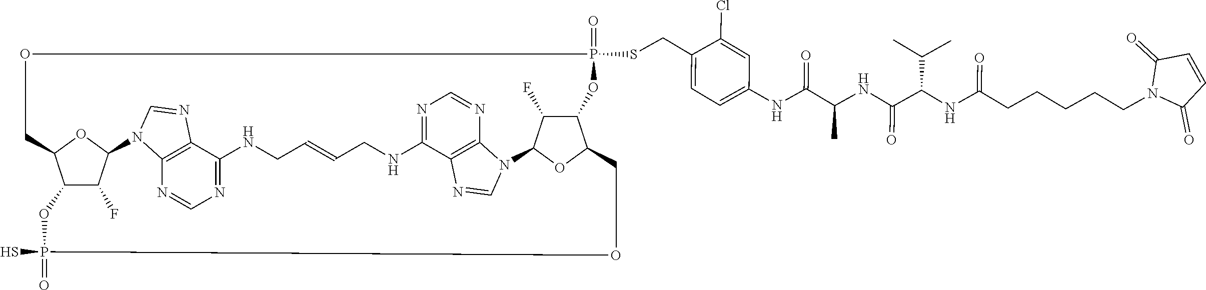 [SMILES: CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)c(Cl)c1] |
| LP41 | 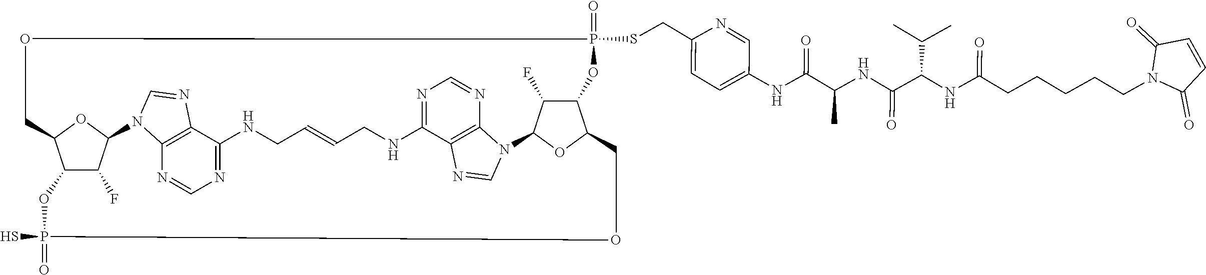 [SMILES: CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)nc1] |
| LP42 | 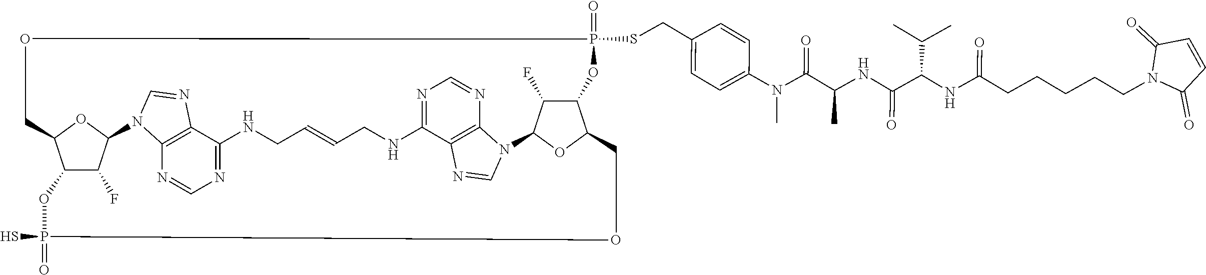 |
| LP43 | 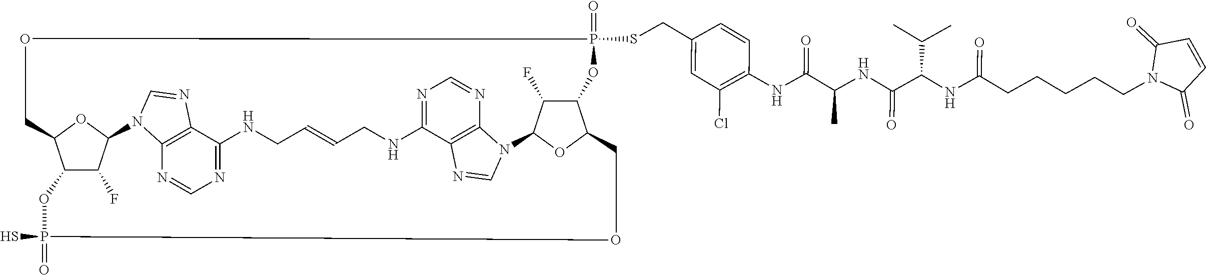 |
| LP44 | 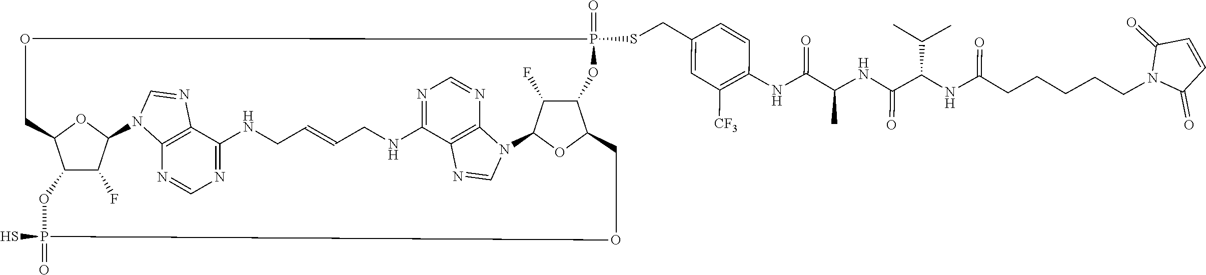 |
| LP45 | 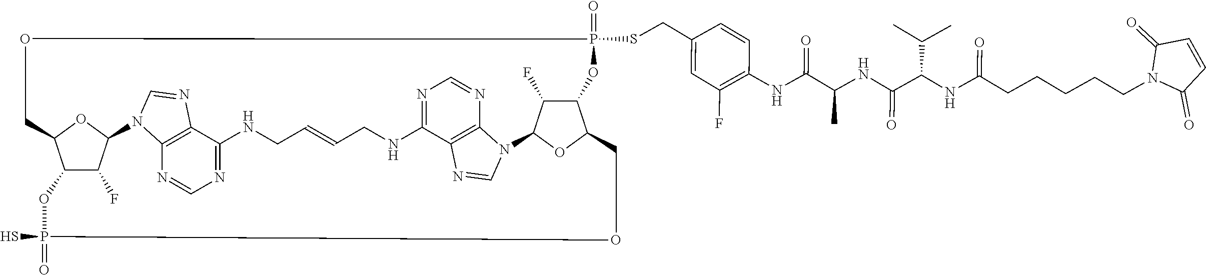 |
| LP46 | 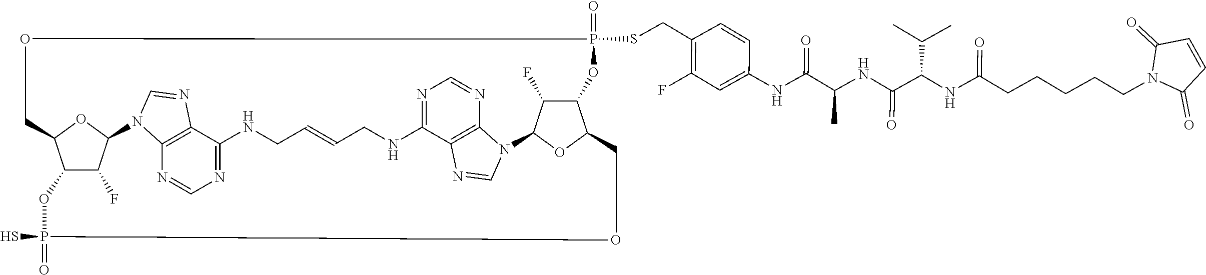 |
| LP47 | 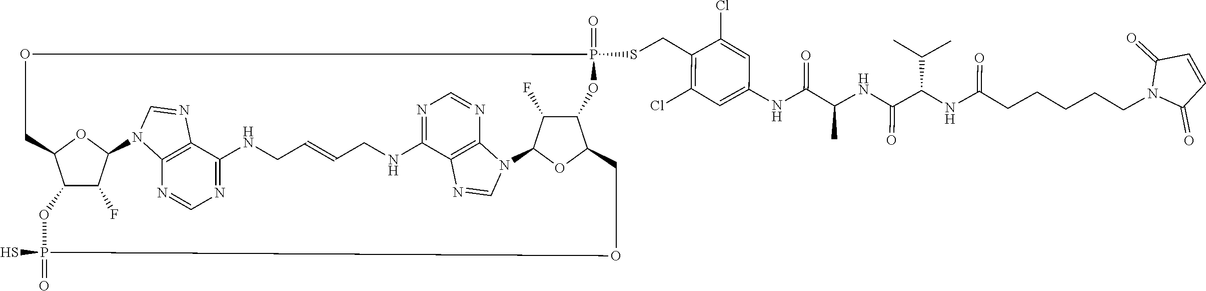 |
| LP48 | 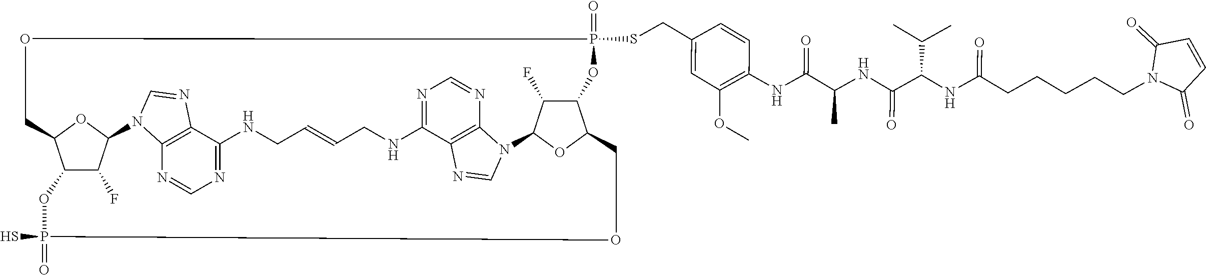 |
| LP49 | 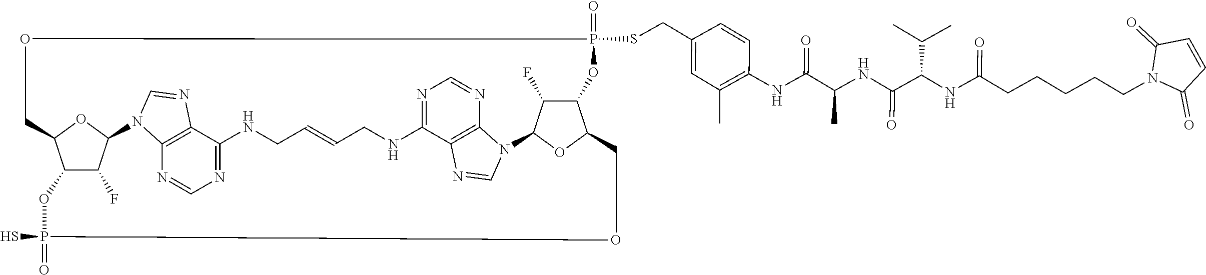 |
| LP50 | 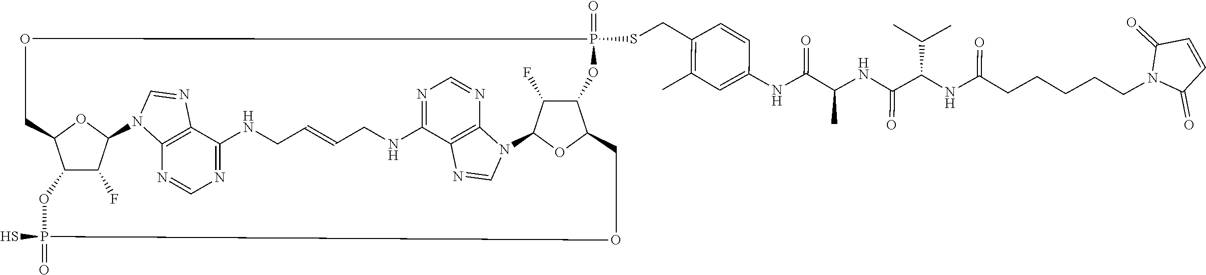 |
| LP51 | 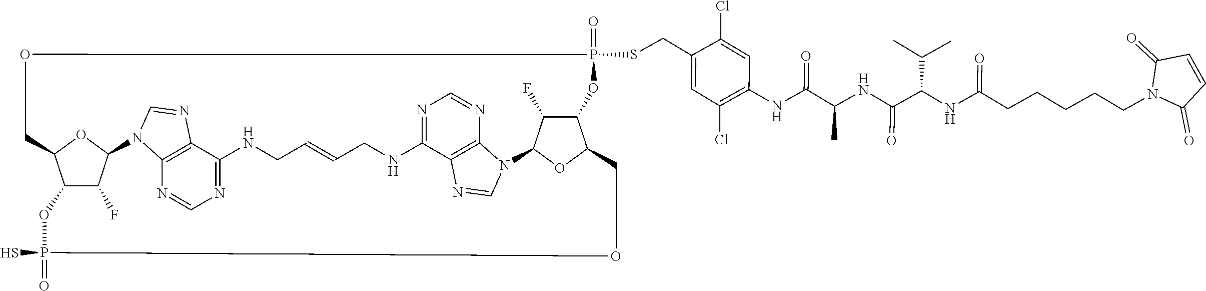 |
| LP52 | 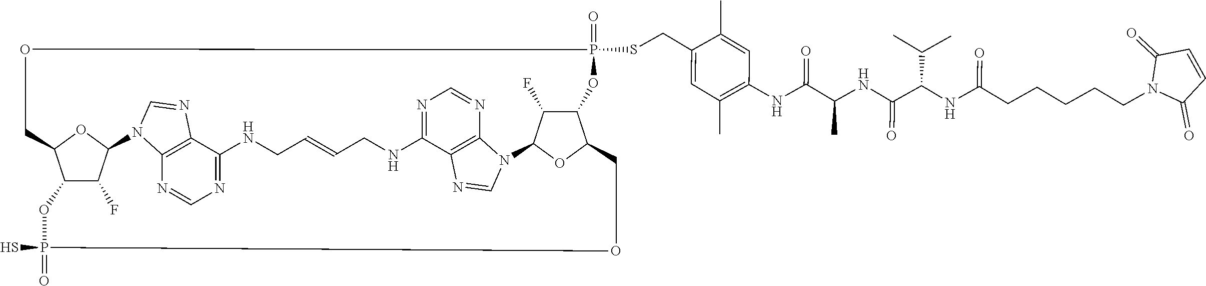 |
| LP53 | 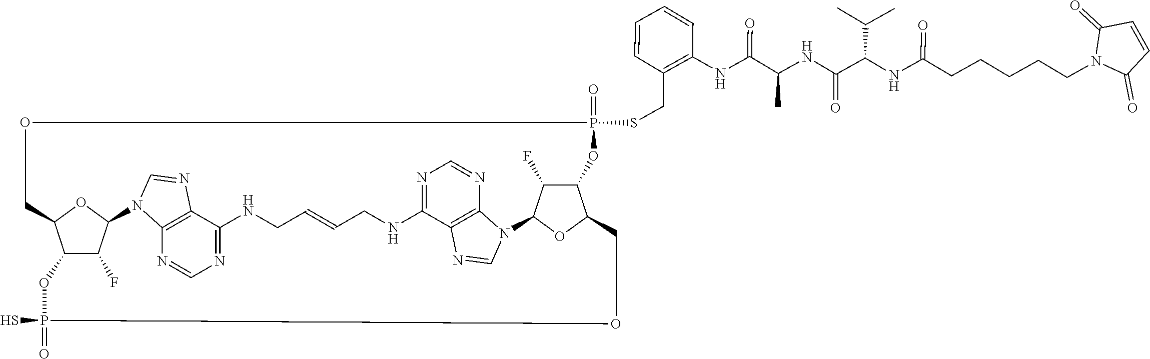 |

TABLE 16
Exemplary N-attached Linker-Drug Conjugates

| | |
|---|---|
| LP1 | 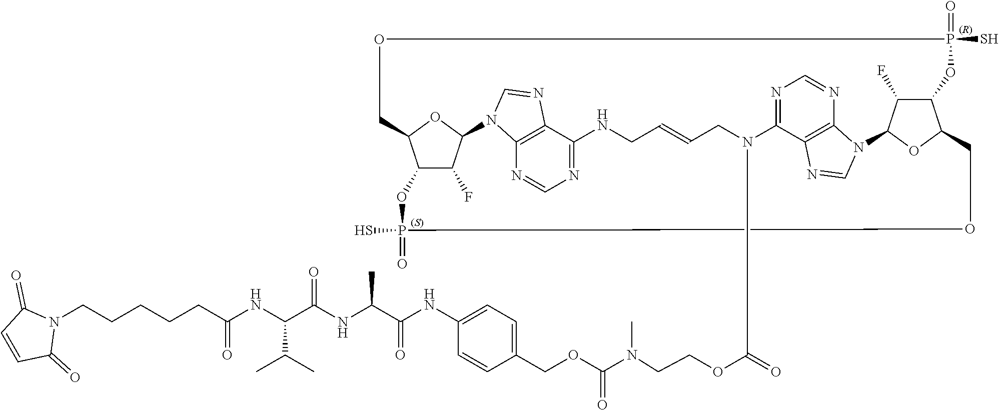 |
| LP2 | 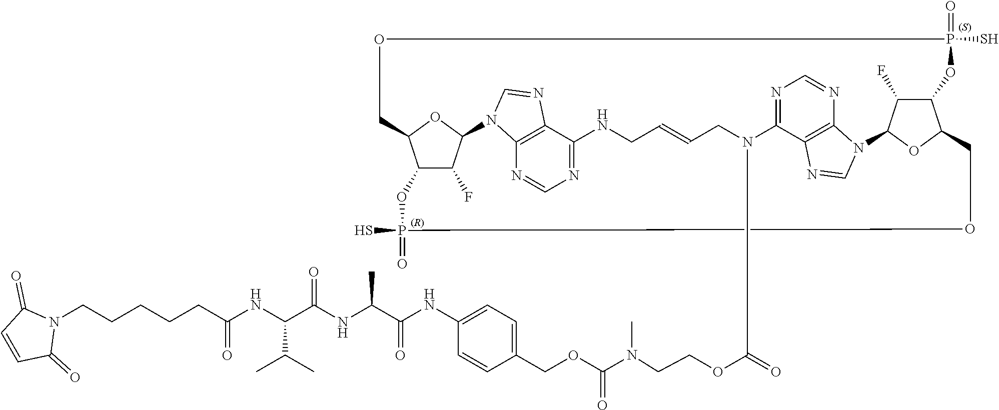 |
| LP4 | 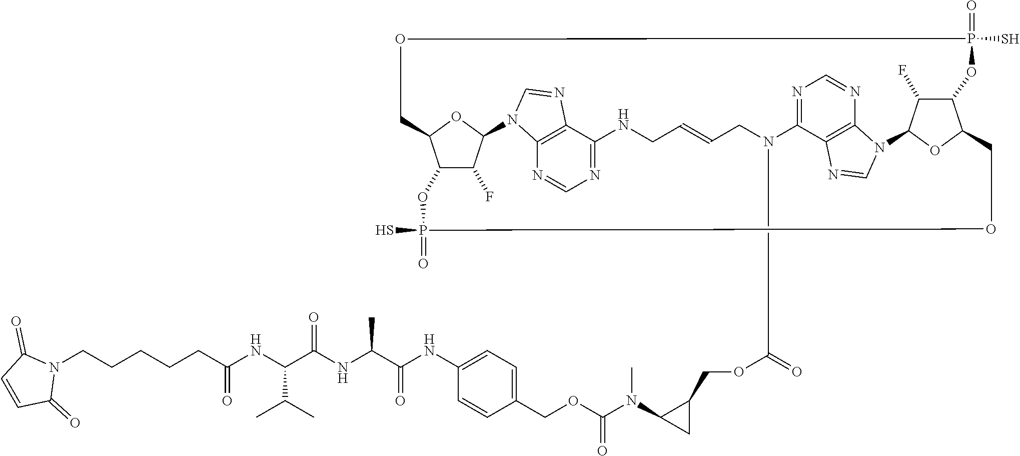 [SMILES: CC(C)[C@H](NC(=O)CCCCCN1C(=O)C=CC1=O)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP5 | 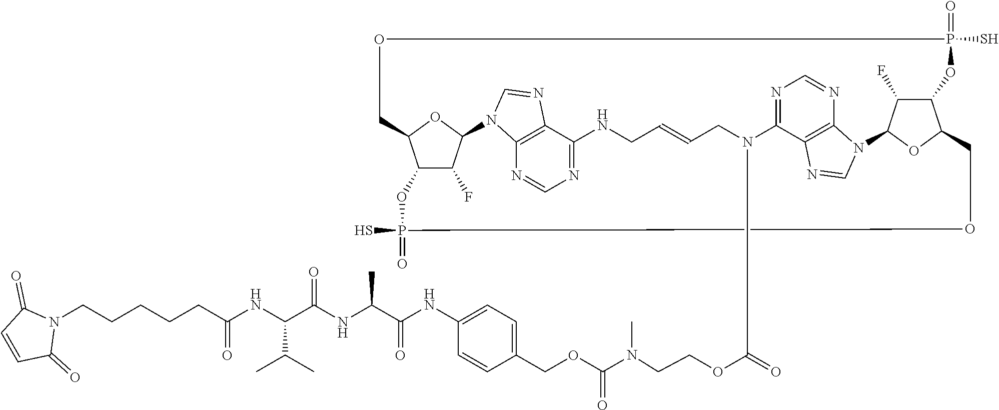 [SMILES: CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP6 | 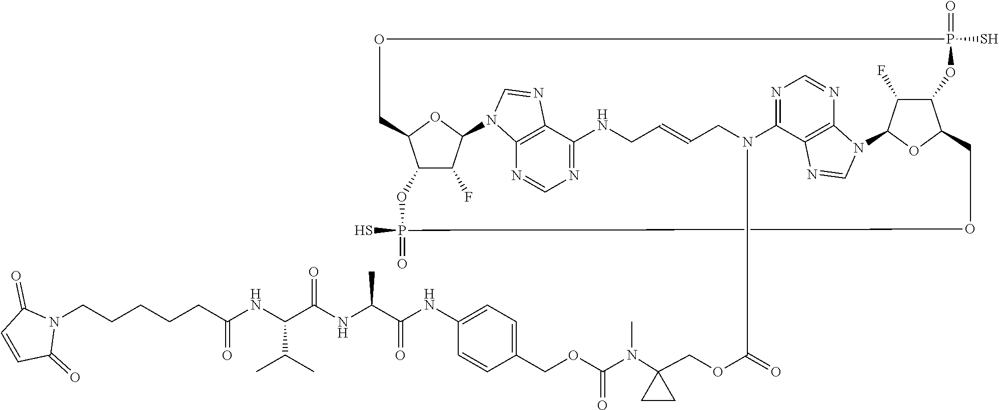 [SMILES: CC(C)[C@H](NC(=O)CCCCCN1C(=O)C=CC1=O)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1] |
| LP7 | 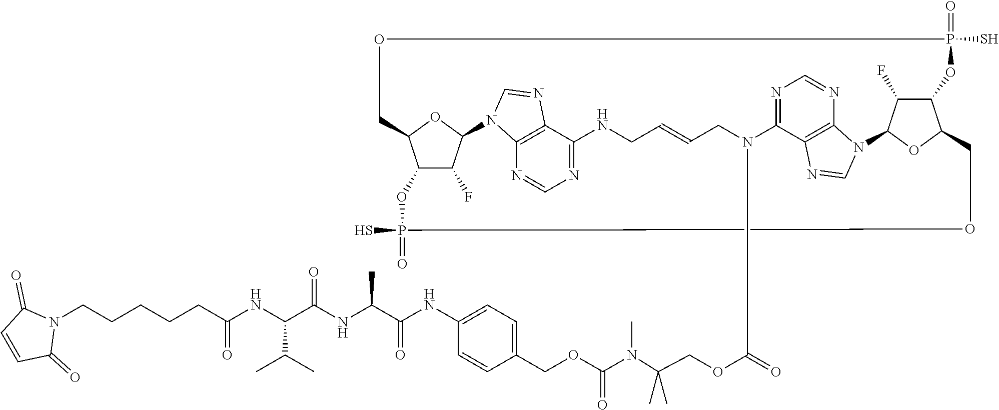 [SMILES: CC(C)[C@H](NC(=O)CCCCCN1C(=O)C=CC1=O)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP8 | 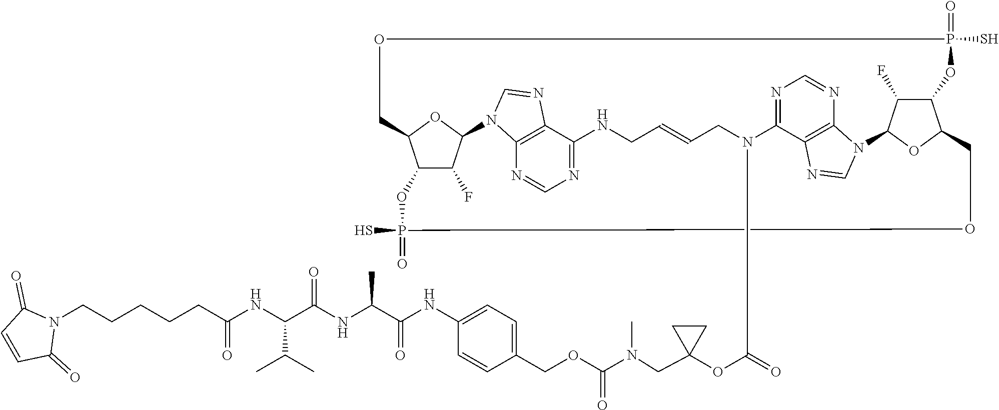 [SMILES: CC(C)[C@H](NC(=O)CCCCCN1C(=O)C=CC1=O)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1] |
| LP9 | 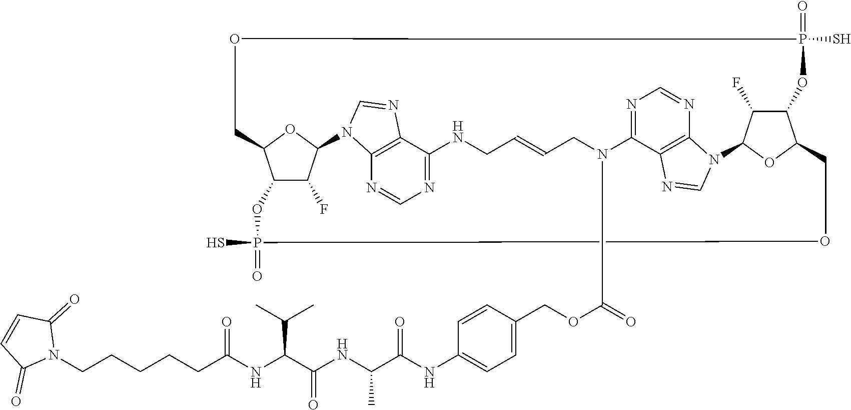 [SMILES: CC(C)[C@H](NC(=O)CCCCCN1C(=O)C=CC1=O)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP10 | 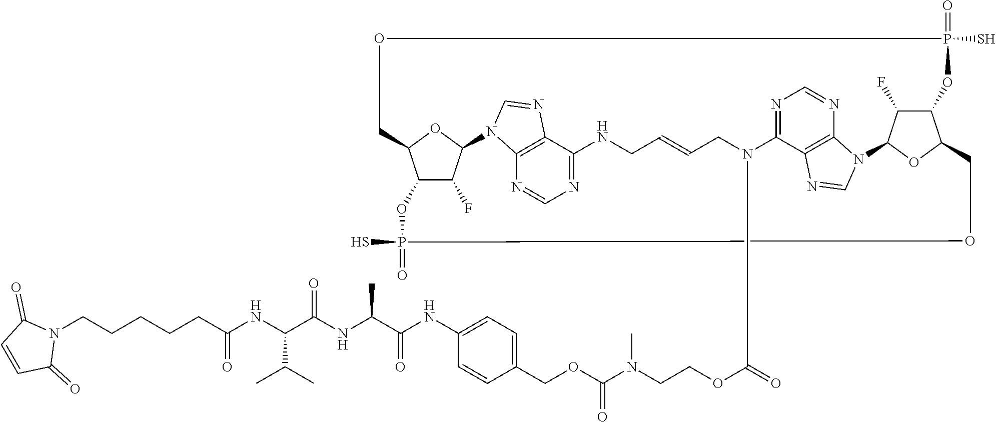 [SMILES: CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP11 | 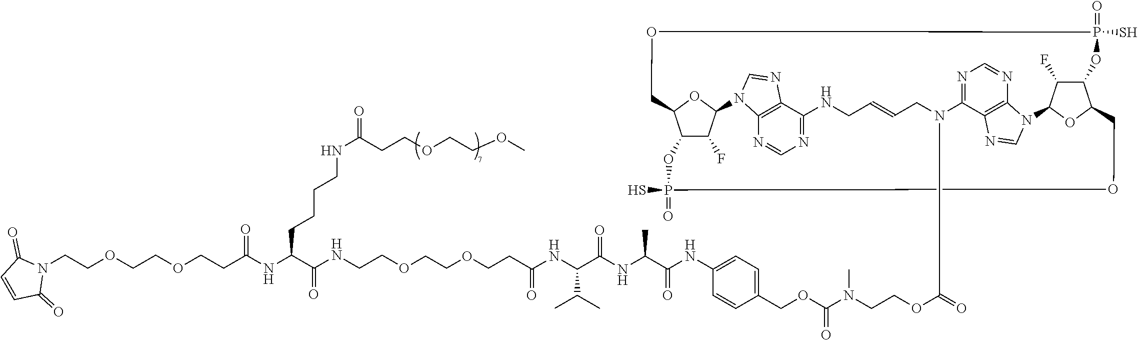 |
| LP12 | 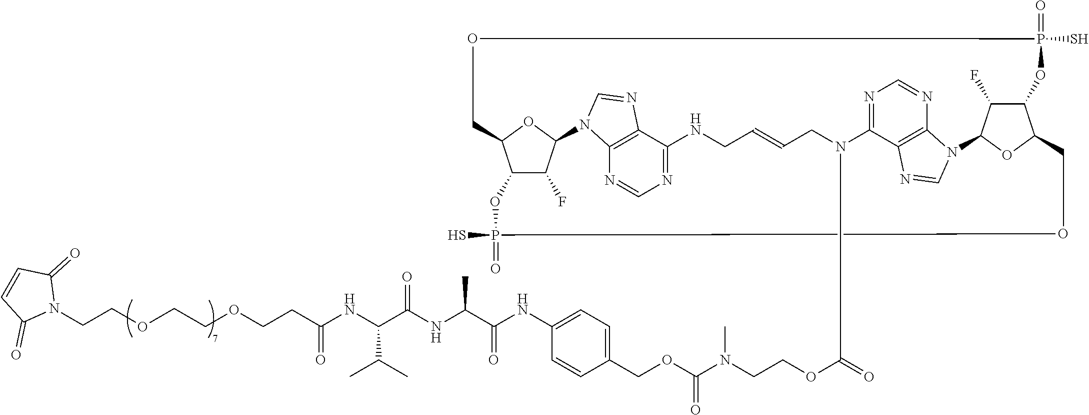 |
| LP13 | 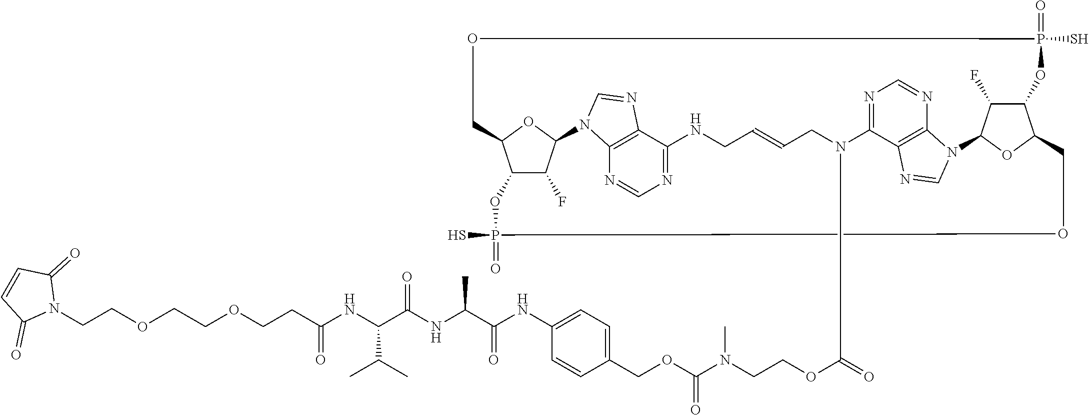 [SMILES: CC(C)[C@H](NC(=O)CCOCCOCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP14 | 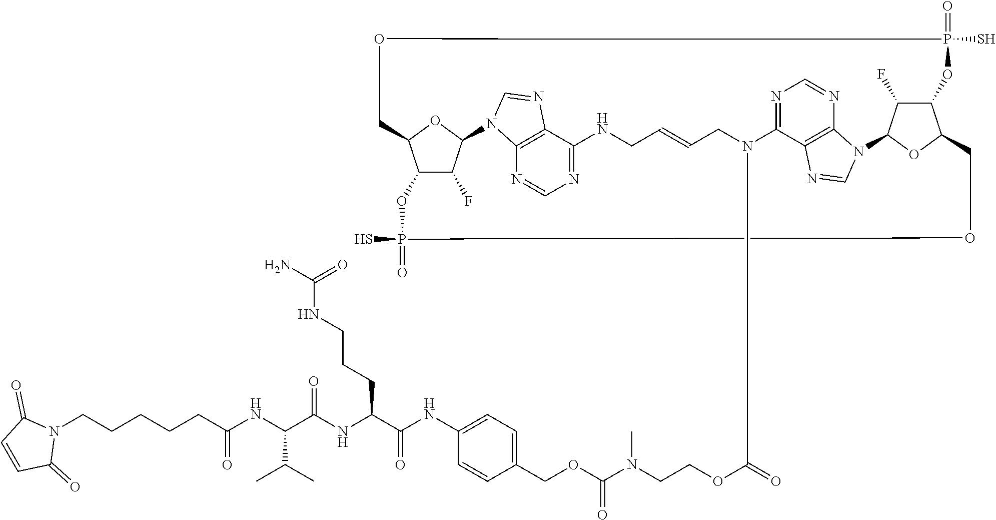 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP15 | 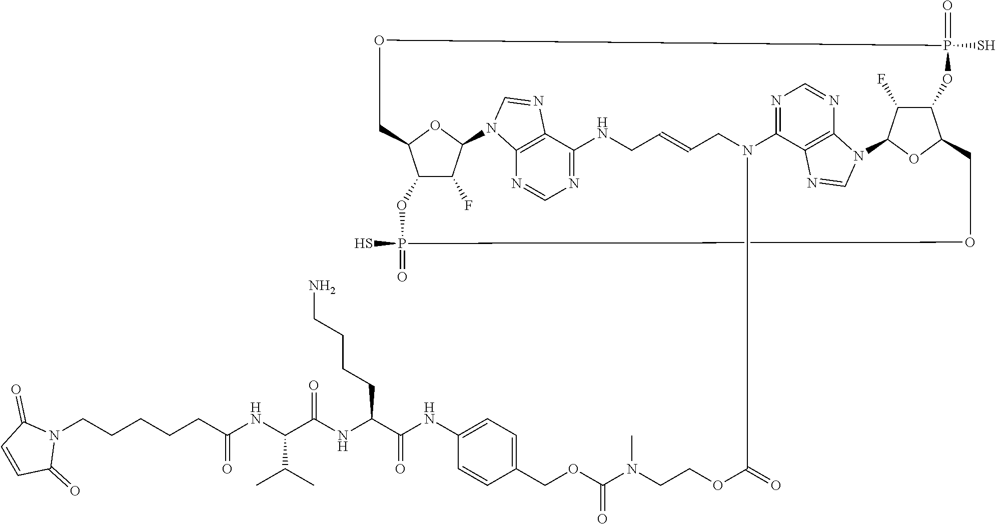 [SMILES: CC(C)[C@H](NC(=O)CCCCCN1C(=O)C=CC1=O)C(=O)N[C@@H](CCCCN)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP16 | 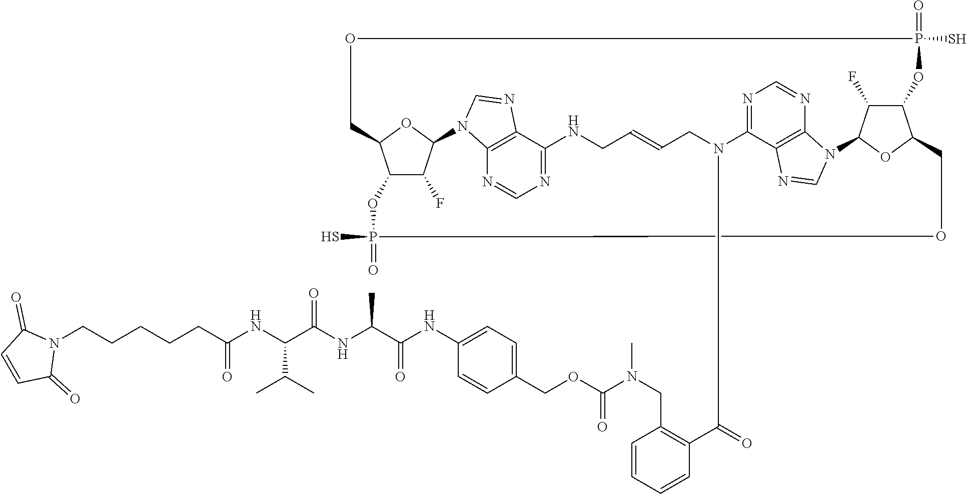 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP17 | 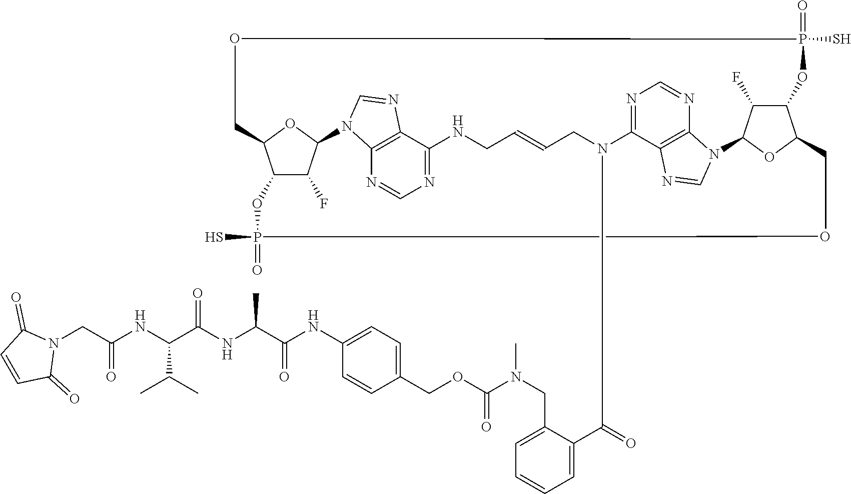 [SMILES: CC(C)[C@H](NC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP18 | 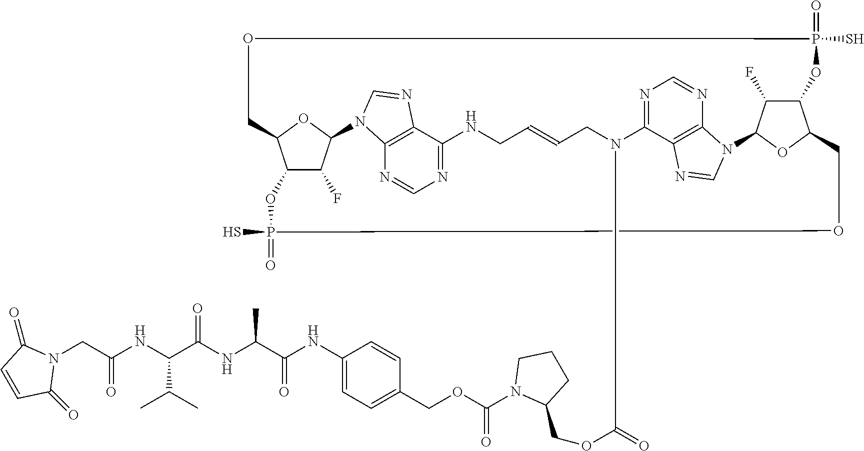 [SMILES: CC(C)[C@H](NC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP19 | 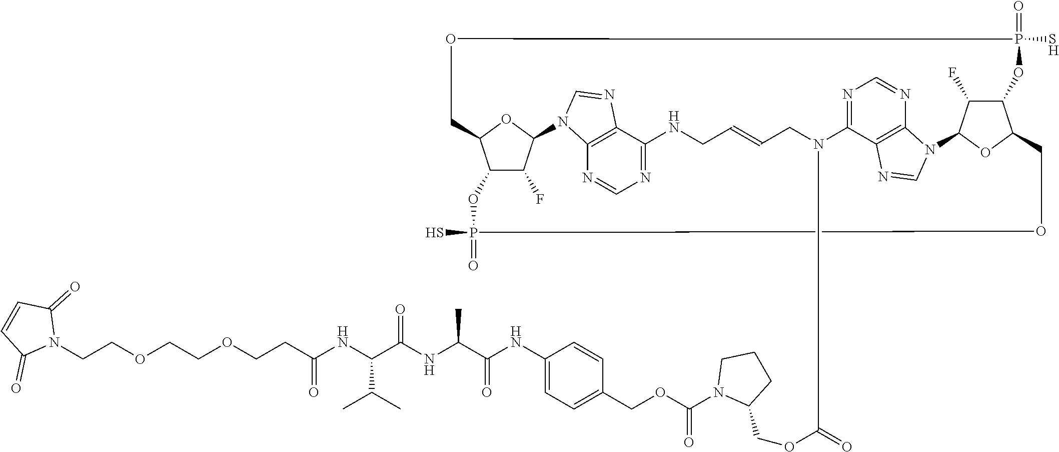 [SMILES: CC(C)[C@H](NC(=O)CCOCCOCCN1C(=O)C=CC1=O)C(=O)N[C@@H](C)C(=O)Nc1ccc(COC(=O)N2CCC[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP20 | 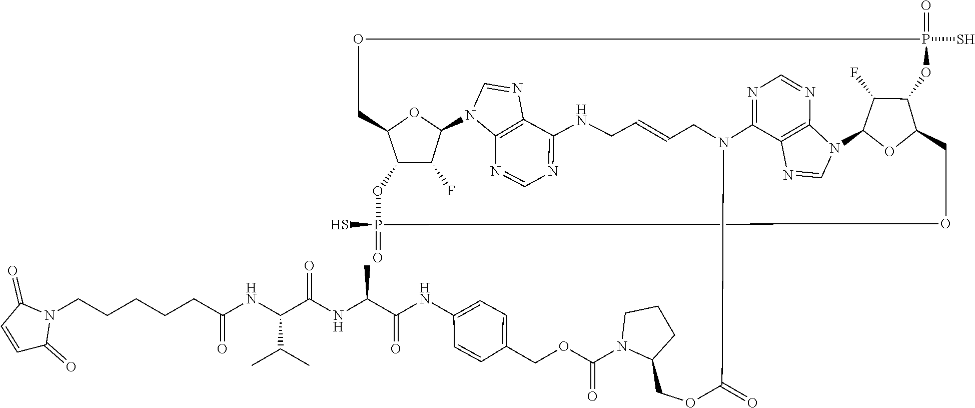 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP21 | 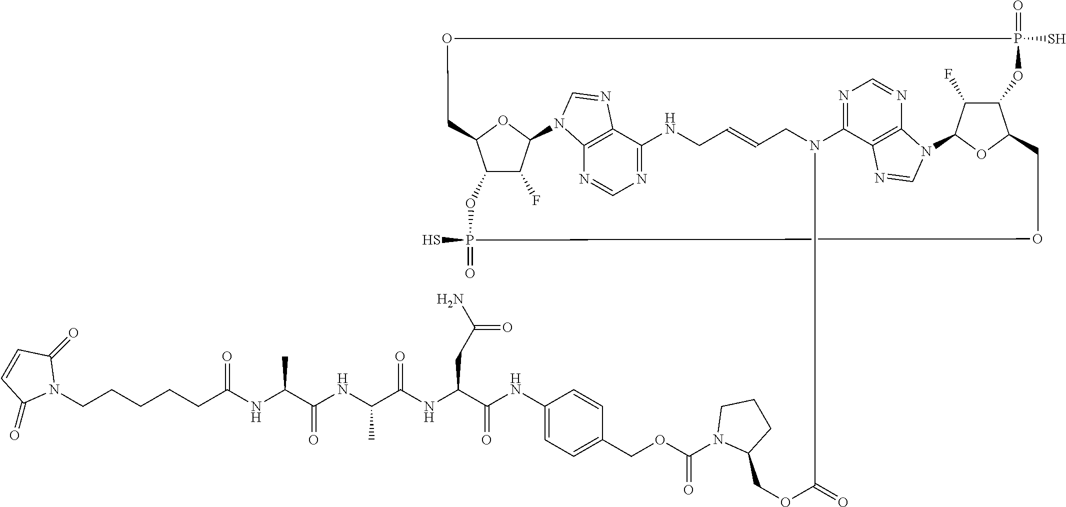 [SMILES: C[C@H](NC(=O)CCCCCN1C(=O)C=CC1=O)C(=O)N[C@@H](C)C(=O)N[C@@H](CC(N)=O)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP22 | 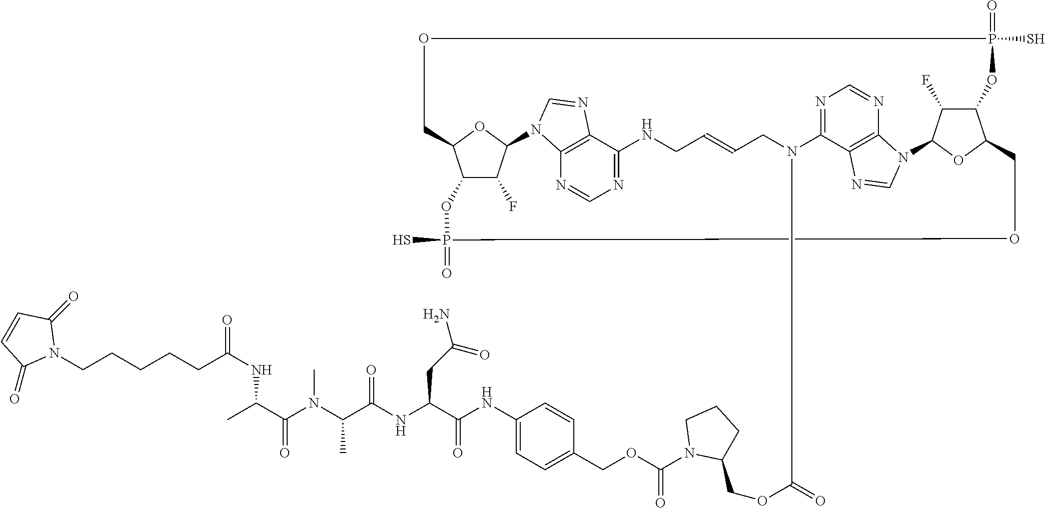 |
| LP23 | 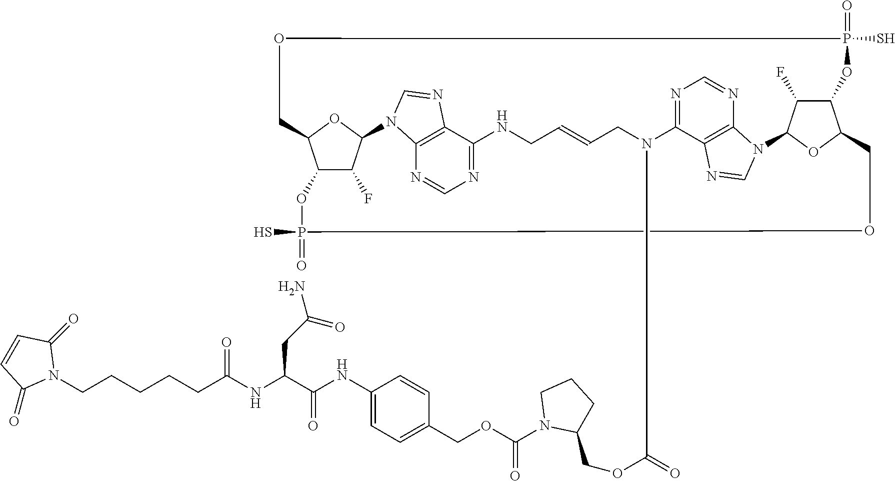 [SMILES: NC(=O)C[C@H](NC(=O)CCCCCN1C(=O)C=CC1=O)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP24 (SEQ ID NO: 144) | 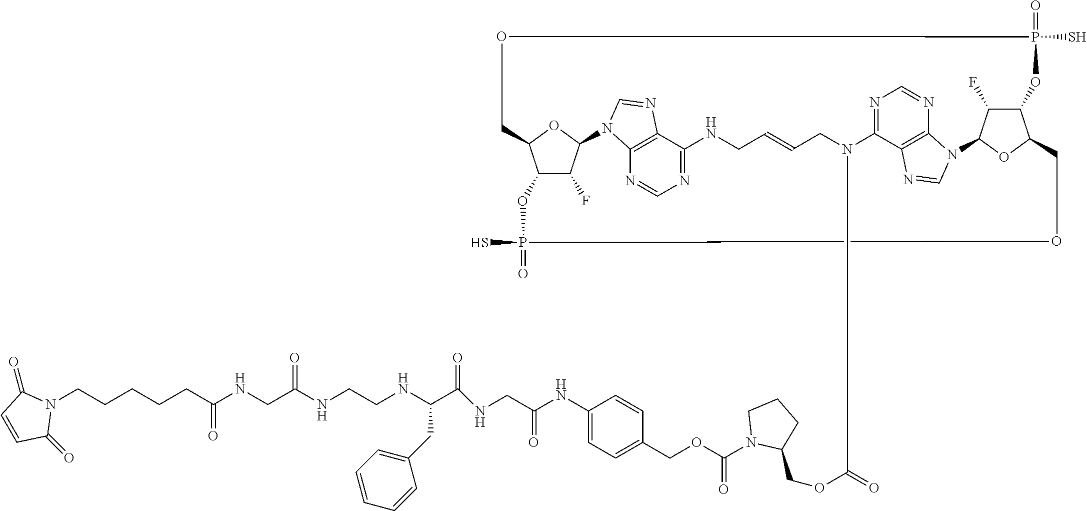 [SMILES: O=C(CCCCCN1C(=O)C=CC1=O)NCC(=O)NCCN[C@@H](Cc1ccccc1)C(=O)NCC(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP25 | 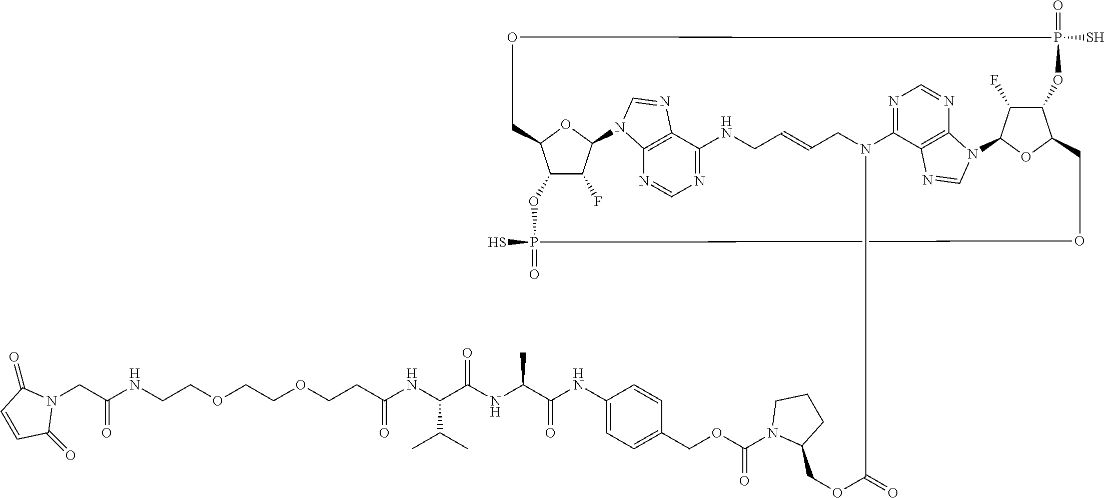 [SMILES: CC(C)[C@H](NC(=O)CCOCCOCCNC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP26 | 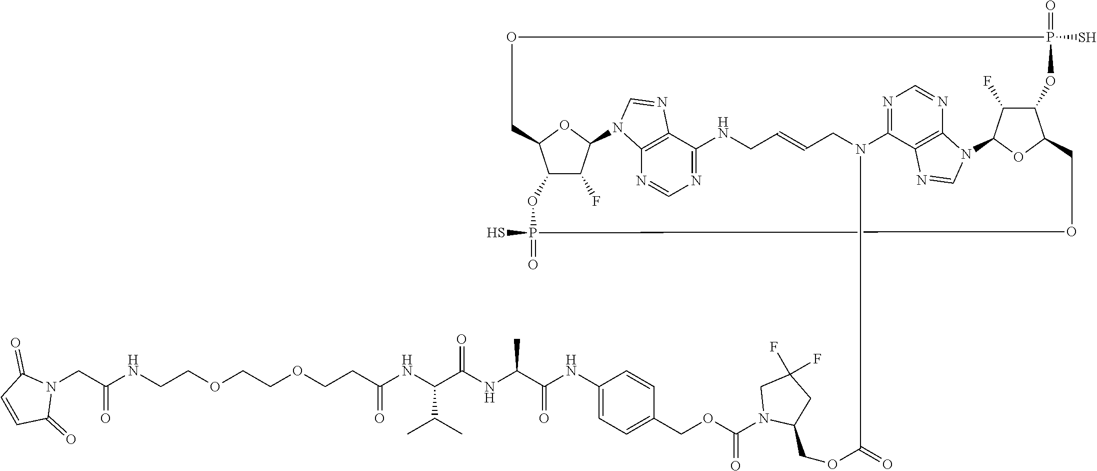 |
| LP27 | 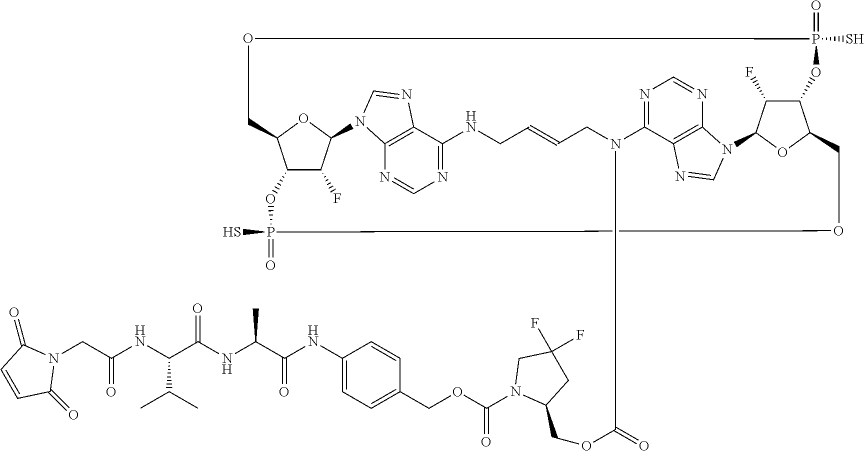 [SMILES: CC(C)[C@H](NC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP28 | 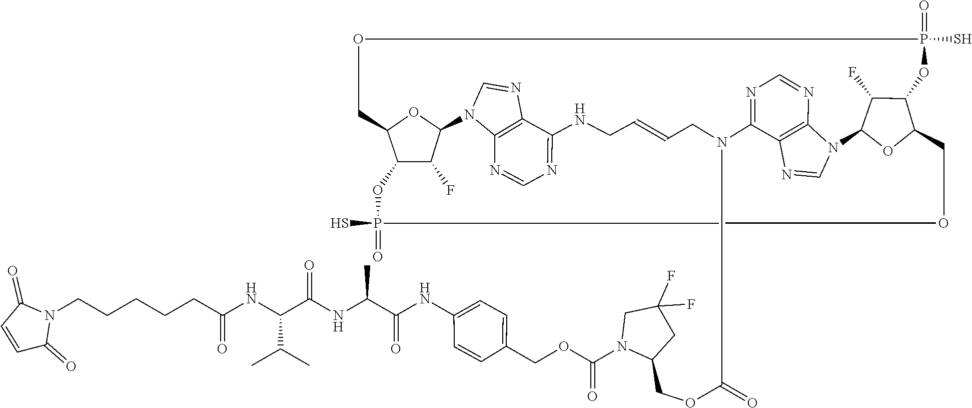 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] |
| LP29 | 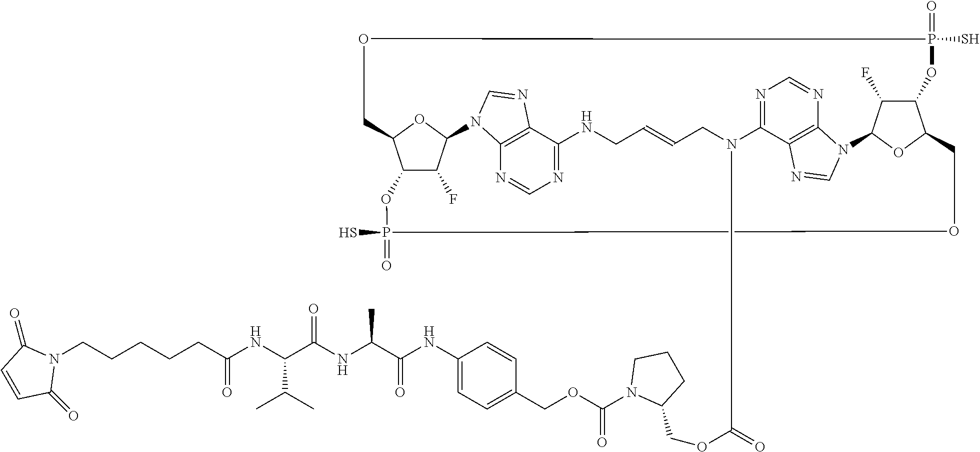 |
| LP30 | 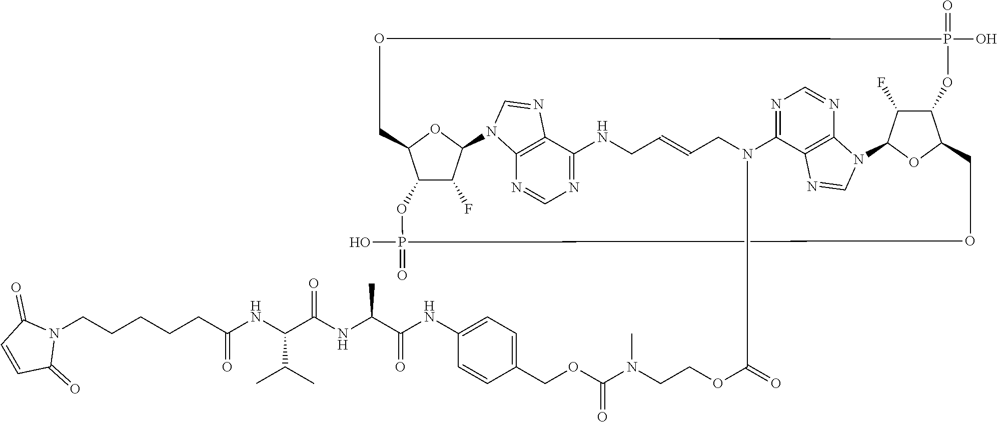 [SMILES: CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4COP(=O)(O)O[C@H]5[C@@H](F)[C@@H](O[C@@H]5COP(=O)(O)O[C@H]4[C@H]3F)n3cnc4c2ncnc43)cc1] |
| LP31 | 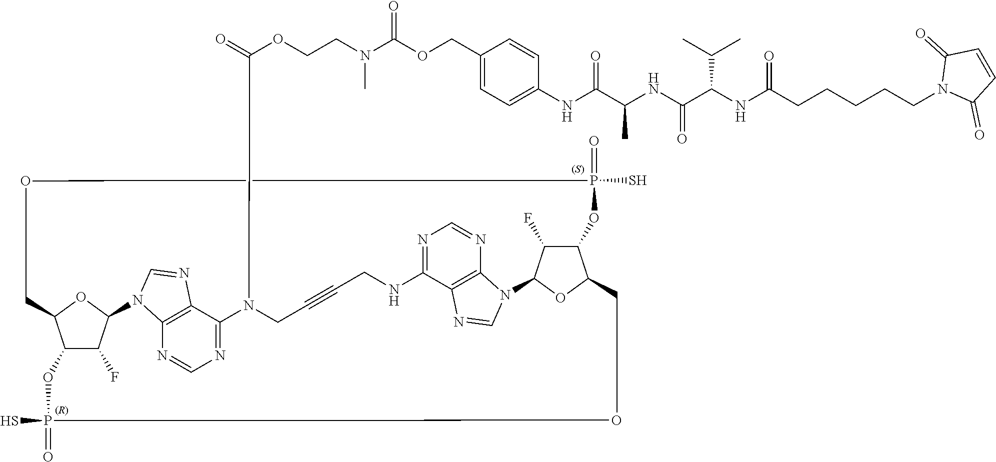 |
| LP32 | 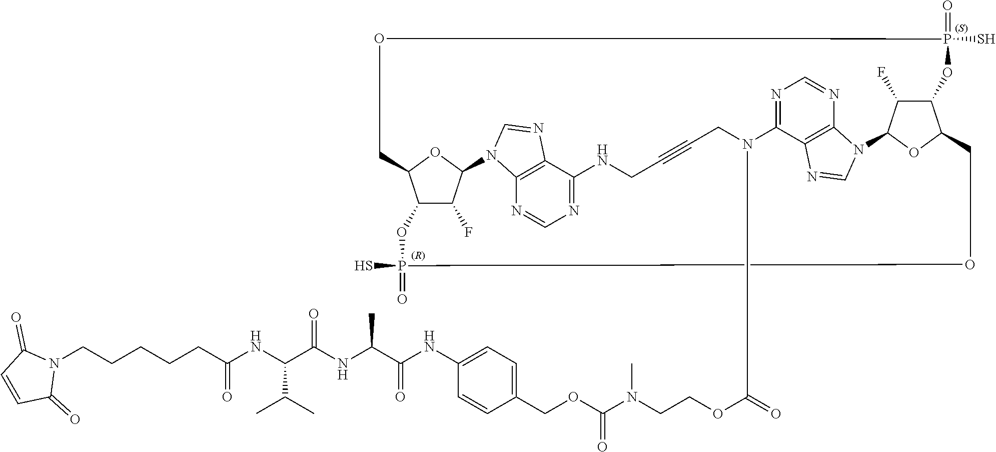 |

In some embodiments, an exemplary linker-drug conjugate or a salt thereof may be referred to as “LP3” and has the structure of LP3 shown below:

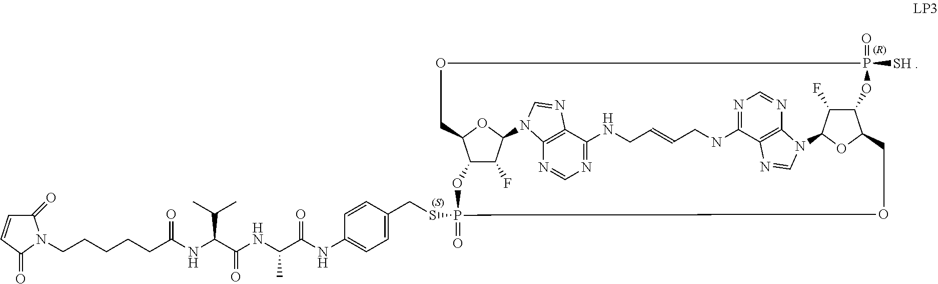

In some embodiments, an exemplary linker-drug conjugate or a salt thereof may be referred to as “LP1” and has the structure of LP1 shown below:

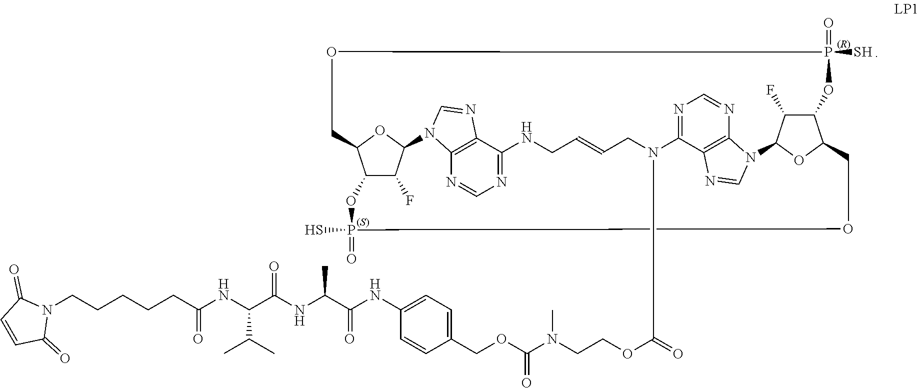

In some embodiments, an exemplary linker-drug conjugate or a salt thereof may be referred to as “LP2” and has the structure of LP2 shown below:

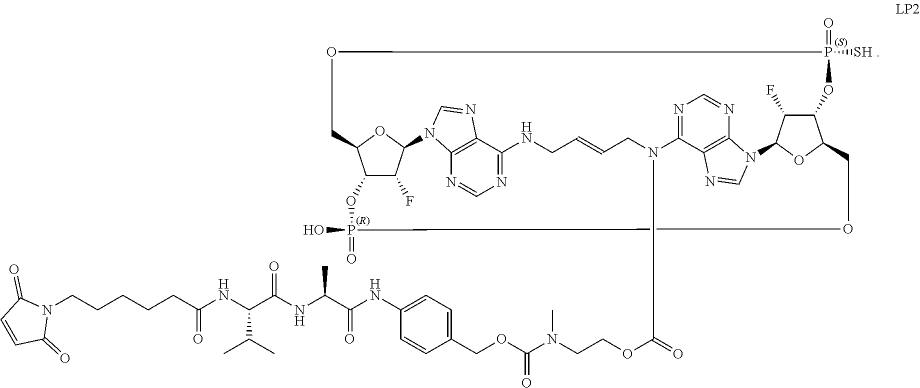

In some embodiments, an exemplary linker-drug conjugate or a salt thereof has the structure of LP16 shown below:

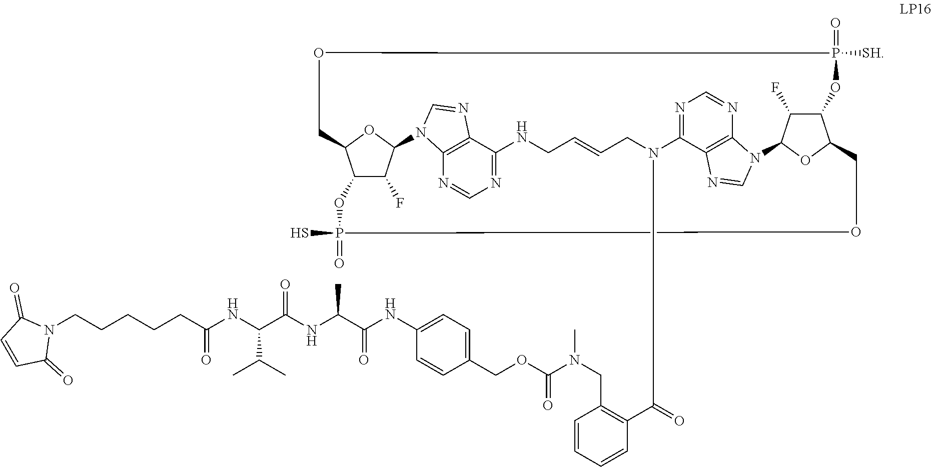 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, an exemplary linker-drug conjugate or a salt thereof has the structure of LP20 shown below:

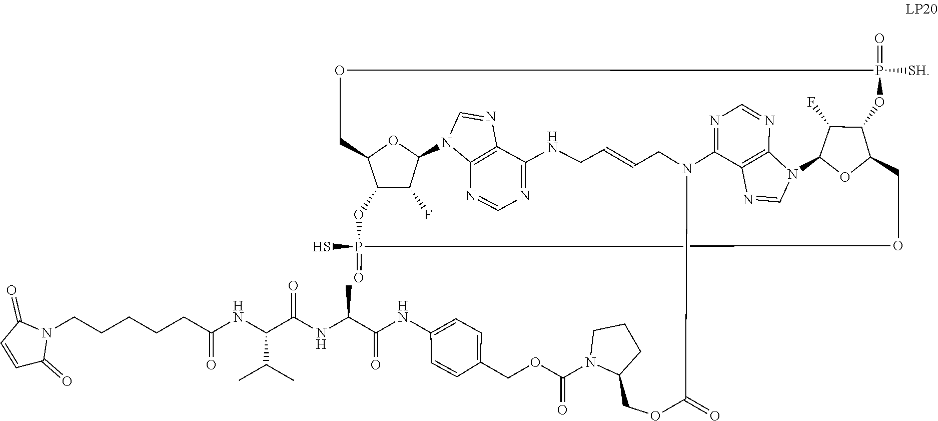 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

some embodiments, an exemplary linker-drug conjugate or a salt thereof has the structure of LP26 shown below:

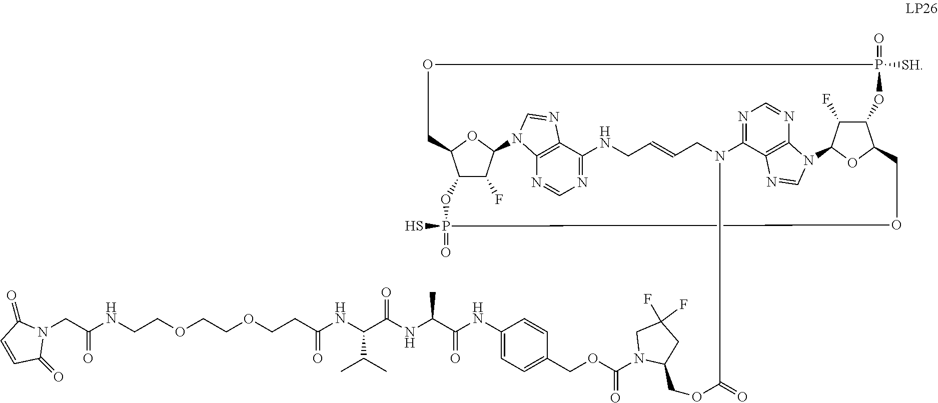 [SMILES: CC(C)[C@H](NC(=O)CCOCCOCCNC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, an exemplary linker-drug conjugate or a salt thereof has the structure of LP28 shown below:

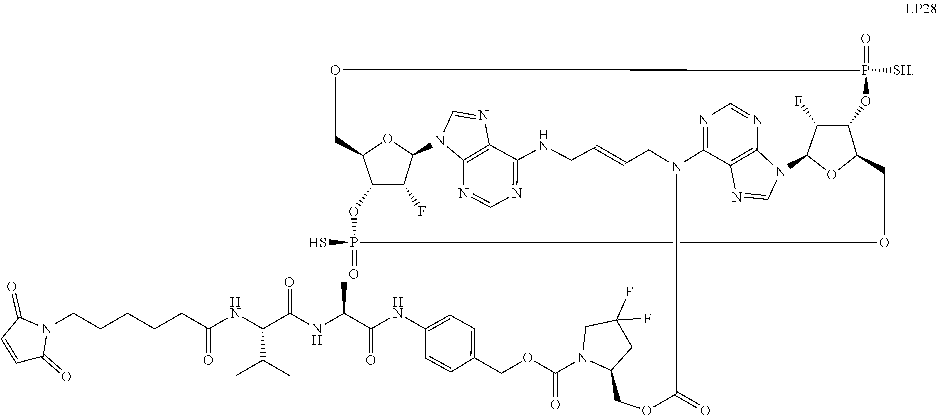 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

In some embodiments, a linker-payload disclosed herein, e.g., LP1, LP2, LP16, LP20, LP26, LP28, or LP3, has improved properties over prior linker-STING agonist conjugates. In some embodiments, a linker-payload disclosed herein, e.g., LP1, LP2, LP3, LP16, LP20, LP26, or LP28, has superior plasma stability over prior art linker-STING agonist conjugates. In some embodiments, a linker-payload disclosed herein, e.g., LP1, LP2, LP3, LP16, LP20, LP26, or LP28, has superior in vivo anti-tumor activity over prior art linker-STING agonist conjugates. In some embodiments, a linker-payload disclosed herein, e.g., LP1, LP2, LP3, LP16, LP20, LP26, or LP28, has superior tolerability in vivo over prior art linker-STING agonist conjugates.

In some embodiments of linker-payload conjugates disclosed herein, wherein D is a STING agonist, e.g., a compound of Formula (III), Formula (IV), or Table 14, e.g., Compound 1, and L is conjugated to D at the N-34 nitrogen or the N-39 nitrogen (e.g., LP16, LP20, LP26, or LP28), the linker-payload conjugate demonstrates superior properties (e.g., plasma stability, in vitro immune responses, in vivo anti-tumor activity, tolerability, stimulation of an anti-immune response in the tumor microenvironment) compared to other linker-payload conjugates comprising a compound of Formula (III), Formula (IV), or Table 14 that are conjugated to D at alternative attachment points, e.g., at a sulfur, e.g., S-2 or 5-14. Exemplary evidence of the superior benefits of such linker-payload conjugates are shown in Examples 4, 9, 12, 14, and 15.

In some embodiments of linker-payload conjugates disclosed herein, wherein L comprises a spacer unit comprising Formula (II), the linker-payload conjugate demonstrates superior properties (e.g., improved conjugation stability, improved plasma stability, in vivo anti-tumor activity) compared to other linker-payload conjugates comprising alternative spacer units. In some embodiments, without being bound by theory, benefits of using a linker comprising Formula (II) with a STING agonist disclosed herein, e.g., a compound of Formula (III), Formula (IV), or Table 14, e.g., Compound 1, may include improved conjugation stability, improved plasma stability, and in vivo anti-tumor activity. In some embodiments, a linker-payload conjugate comprising a linker comprising Formula (II) and a payload comprising a STING agonist disclosed herein, e.g., a compound of Formula (III), Formula (IV), or Table 14 demonstrates superior properties when conjugated to an anti-PSMA antibody disclosed herein. Exemplary evidence of the superior benefits of such linker-payload conjugates, e.g., benefits that may be afforded when conjugated to a variety of different antibodies, is shown in Examples 4, 9, 12, and 15.

In some embodiments, an ADC disclosed herein comprises a cleavable linker and an internalizing anti-PSMA antibody or antigen-binding fragment thereof as described herein. In some embodiments, the anti-PSMA antibody or antigen-binding fragment thereof comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system. In some embodiments, the anti-PSMA antibody or antigen-binding fragment thereof comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 33 (LCDR1), SEQ ID NO: 36 (LCDR2), and SEQ ID NO: 37 (LCDR3), as defined by the Kabat numbering system. In some embodiments, the anti-PSMA antibody or antigen-binding fragment thereof comprises three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

In some embodiments, the anti-PSMA antibody or antigen-binding fragment thereof comprises a heavy chain variable region comprising an amino acid sequence of SEQ ID NO: 1, and a light chain variable region comprising an amino acid sequence of SEQ ID NO: 15. In some embodiments, the anti-PSMA antibody or antigen-binding fragment thereof comprises a heavy chain variable region comprising an amino acid sequence of SEQ ID NO: 2, and a light chain variable region comprising an amino acid sequence of SEQ ID NO: 15. In some embodiments, the anti-PSMA antibody or antigen-binding fragment thereof comprises a heavy chain variable region comprising an amino acid sequence of SEQ ID NO: 3, and a light chain variable region comprising an amino acid sequence of SEQ ID NO: 15. In some embodiments, the anti-PSMA antibody or antigen-binding fragment thereof comprises a heavy chain variable region comprising an amino acid sequence of SEQ ID NO: 14, and a light chain variable region comprising an amino acid sequence of SEQ ID NO: 15. In some embodiments, the anti-PSMA antibody or antigen-binding fragment thereof comprises a heavy chain variable region comprising an amino acid sequence of SEQ ID NO: 14, and a light chain variable region comprising an amino acid sequence of SEQ ID NO: 19.

In some embodiments, p is from 1 to 12, or 2 to 11. In some embodiments, p is from 1 to 8.

In some embodiments, p is from 4 to 11. In some embodiments, p is from 4 to 8. In some embodiments, p is 2. In some embodiments, p is 4. In some embodiments, p is 7. In some embodiments, p is 11.

The present disclosure includes methods of producing the described linker-drug conjugates. The linker-drug conjugates comprise a linker and a drug moiety and can be prepared using a linker having reactive functionalities for covalently attaching the linker to the drug moiety. In some embodiments, the method of producing the linker-drug conjugates comprises reacting a drug or a salt thereof with an activated linker.

In some embodiments, the drug that is reacted with an activated linker is a compound disclosed in Table 14; an isomer of the compound; a deuterated derivative of the compound or isomer; or a salt of the compound, isomer, or deuterated derivative. In some embodiments, the drug is a sodium salt of a compound disclosed in Table 14. In some embodiments, the drug is a diammonium salt of a compound disclosed in Table 14. In some embodiments, the drug is a dialkylammonium salt of a compound disclosed in Table 14. In some embodiments, the drug is a bis(triethylammonium) salt of a compound disclosed in Table 14.

In some embodiments, the activated linker that is reacted with a compound disclosed in Table 14; an isomer of the compound; a deuterated derivative of the compound or isomer; or a salt of the compound, isomer, or deuterated derivative is

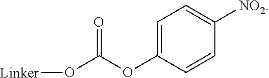

In some embodiments, the activated linker that is reacted with a compound disclosed in Table 14; an isomer of the compound; a deuterated derivative of the compound or isomer; or a salt of the compound, isomer, or deuterated derivative is

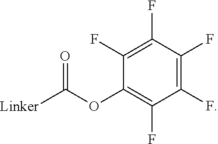

In some embodiments, the linker moiety of the activated linker comprises a linker of the disclosure, e.g., a linker disclosed above, e.g., as disclosed in this section.

In some embodiments, the method of producing the linker-drug conjugates comprises reacting a compound disclosed in Table 14; an isomer of the compound; a deuterated derivative of the compound or isomer; or a salt of the compound, isomer, or deuterated derivative with an activated linker of the disclosure. In some embodiments, the reaction of the compound, isomer, deuterated derivative, or salt is performed in the presence of an organometallic base. In some embodiments, the organometallic base is selected from LDA, NaHMDS, LiHMDS, and KHMDS. In some embodiments, the organometallic base is LiHMDS.

In some embodiments, the method of producing the activated linker

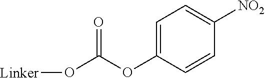

comprises reacting a linker of the disclosure with 4-nitrophenyl carbonochloridate. In some embodiments, the reaction of the linker with 4-nitrophenyl carbonochloridate is performed in the presence of a base. In some embodiments, the base is pyridine.

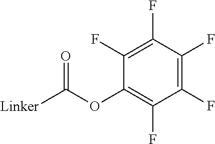

In some embodiments, the method of producing the activated linker comprises reacting a linker of the disclosure with pentafluorophenol. In some embodiments, the reaction of the linker with pentafluorophenol is performed in the presence of a peptide coupling reagent. In some embodiments, the peptide coupling reagent is DCC.

In some embodiments, the activated linker is used in a method of producing an L-D conjugate (V):

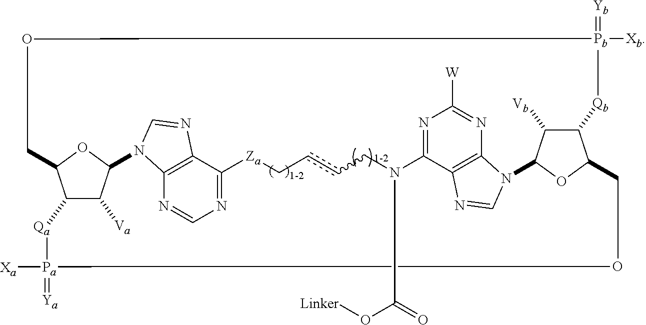

In some embodiments, the method of producing L-D conjugate (V) comprises reacting a compound of Formula (III) or a salt thereof with the activated linker

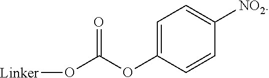

In some embodiments, Z_{b} is NH. In some embodiments, P_{b} has (S)-configuration, and the activated linker reacts with Z_{b} preferentially. “Preferentially,” as used herein (unless context indicates otherwise), refers to more than 70% of a reaction, e.g., 70% of the activated linker reacting with the Z_{b} nitrogen over the Zₐ nitrogen.

In some embodiments, the activated linker reacts with the Z_{b} nitrogen more than 95%, more than 90%, more than 85%, more than 80%, more than 75%, or more than 70% over reaction with the Zₐ nitrogen. In some embodiments, the activated linker reacts with the Z_{b} nitrogen more than 95% over reaction with the Zₐ nitrogen. In some embodiments, the activated linker reacts with the Z_{b} nitrogen more than 90% over reaction with the Zₐ nitrogen. In some embodiments, the activated linker reacts with the Z_{b} nitrogen more than 85% over reaction with the Zₐ nitrogen. In some embodiments, the activated linker reacts with the Z_{b} nitrogen more than 80% over reaction with the Zₐ nitrogen. In some embodiments, the activated linker reacts with the Z_{b} nitrogen more than 75% over reaction with the Zₐ nitrogen. In some embodiments, the activated linker reacts with the Z_{b} nitrogen more than 70% over reaction with the Zₐ nitrogen.

In some embodiments, the activated linker is used in a method of producing an L-D conjugate (VI):

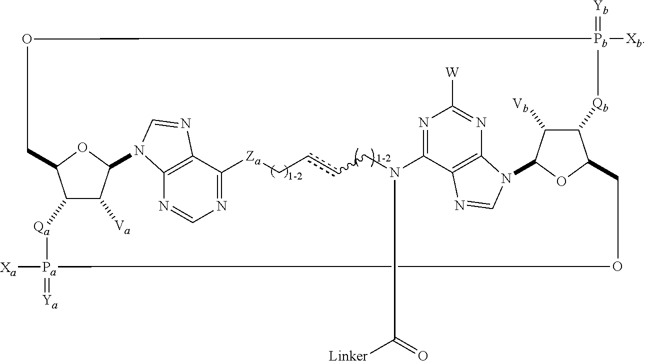

In some embodiments, the method of producing L-D conjugate (VI) comprises reacting a compound of Formula (III) or a salt thereof with the activated linker

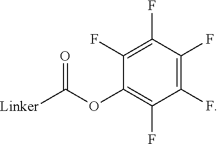

In some embodiments, Z_{b} is NH. In some embodiments, P_{b} has (S)-configuration, and the activated linker reacts with Z_{b} preferentially.

In some embodiments, the activated linker reacts with the Z_{b} nitrogen more than 95%, more than 90%, more than 85%, more than 80%, more than 75%, or more than 70% over reaction with the Zₐ nitrogen. In some embodiments, the activated linker reacts with the Z_{b} nitrogen more than 95% over reaction with the Zₐ nitrogen. In some embodiments, the activated linker reacts with the Z_{b} nitrogen more than 90% over reaction with the Zₐ nitrogen. In some embodiments, the activated linker reacts with the Z_{b} nitrogen more than 85% over reaction with the Zₐ nitrogen. In some embodiments, the activated linker reacts with the Z_{b} nitrogen more than 80% over reaction with the Zₐ nitrogen. In some embodiments, the activated linker reacts with the Z_{b} nitrogen more than 75% over reaction with the Zₐ nitrogen. In some embodiments, the activated linker reacts with the Z_{b} nitrogen more than 70% over reaction with the Zₐ nitrogen.

### Antibody-Drug Conjugates

In various embodiments, an anti-PSMA antibody moiety or an antigen-binding fragment thereof as disclosed herein may be conjugated (i.e., covalently attached, e.g., by a linker) to a drug moiety, wherein the drug moiety when not conjugated to an antibody moiety has a cytotoxic or cytostatic effect. In some embodiments, the drug moiety exhibits reduced or no cytotoxicity when bound in a conjugate but resumes cytotoxicity after cleavage from the linker and antibody moiety.

The development and production of an ADC for use as a human therapeutic agent, e.g., as an oncologic agent, may require more than the identification of an antibody capable of binding to a desired target or targets and attaching to a drug used on its own to treat cancer. Linking the antibody to the drug may have significant and unpredictable effects on the activity of one or both of the antibody and the drug, effects which will vary depending on the type of linker and/or drug chosen. In some embodiments, therefore, the components of the ADC are selected to (i) retain one or more therapeutic properties exhibited by the antibody and drug moieties in isolation, (ii) maintain the specific binding properties of the antibody moiety; (iii) optimize drug loading and drug-to-antibody ratios; (iv) allow targeted tumor cell delivery, e.g., intracellular delivery, of the drug moiety via stable attachment to the antibody moiety; (v) reduce toxicity compared to non-targeted and/or systemic delivery of the drug moiety; (vi) retain ADC stability as an intact conjugate until transport or delivery to a target site; (vii) minimize aggregation of the ADC prior to or after administration; (viii) exhibit in vivo anti-cancer treatment efficacy comparable to or superior to that of the antibody and drug moieties in isolation; (ix) minimize off-target killing by the drug moiety; (x) exhibit desirable pharmacokinetic and pharmacodynamics properties, formulatability, and toxicologic/immunologic profiles; (xi) maintain stimulation of an anti-immune response in the tumor microenvironment; and/or (xii) increase phagocytosis of PSMA-expressing cells by myeloid cells (e.g., macrophages or dendritic cells). Screening each of these properties may be needed to identify an improved ADC for therapeutic use. See, e.g., Ab et al. (2015) Mol. Cancer Ther. 14:1605-13.

In some embodiments, the ADC compounds of the present disclosure have superior stability as an intact conjugate until transported or delivered to a target site compared to ADC compounds comprising other antibodies, e.g., J591 or deJ591, and/or other linkers. In some embodiments, the ADC compounds of the present disclosure are less immunogenic compared to ADC compounds comprising other antibodies, e.g., J591 or deJ591, and/or other linkers.

The ADC compounds of the present disclosure may selectively deliver an effective dose of a cytotoxic or cytostatic agent to cancer cells or to tumor tissue. It has been discovered that the disclosed ADCs have potent cytotoxic and/or cytostatic activity against cells expressing PSMA. In some embodiments, the cytotoxic and/or cytostatic activity of the ADC is dependent on PSMA expression level in a cell. In some embodiments, the disclosed ADCs are particularly effective at killing cancer cells expressing a high level of PSMA, as compared to cancer cells expressing the same antigen at a low level. In some embodiments, the disclosed ADCs are particularly effective at killing high PSMA-expressing cancers such as prostate cancer. In some embodiments, targeted killing of PSMA-expressing cancer cells is improved by the presence or recruitment of myeloid cells (e.g., macrophages and/or dendritic cells).

In some embodiments, ADCs disclosed herein demonstrate PSMA-specific binding on PSMA-expressing cells, e.g., in PSMA-expressing cancers. In some embodiments, upon binding PSMA, the disclosed ADCs are internalized. In some embodiments, release of the drug moiety, e.g., Compound 1, results in STING pathway activation and release of proinflammatory cytokines (e.g., IFNβ). In some embodiments, release of proinflammatory cytokines promotes myeloid cell activation. In some embodiments, release of proinflammatory cytokines stimulates Type I IFN-dependent anti-tumor activity.

In some embodiments, the disclosed ADCs activate myeloid cells, e.g., macrophages or dendritic cells. Without being bound by theory, myeloid cell activation may be a result of phagocytosis of PSMA-expressing cancer cells bound by the disclosed ADCs. In some embodiments, the disclosed ADCs activate macrophages. in some embodiments, the activated macrophages are proinflammatory (M1) macrophages. In some embodiments, tumor-associated macrophages or M2 macrophages undergo proinflammatory activation upon administration of the disclosed ADCs. In some embodiments, the activated macrophages release pro-inflammatory cytokines and chemokines (e.g., TNFα, CXCL10, IL-6, IFNβ, and/or IL-1β). In some embodiments, the activated macrophages promote further myeloid cell activation. In some embodiments, the activated macrophages promote the generation of cytotoxic T cells. In some embodiments, the activated macrophages demonstrate increased phagocytosis of cancer cells. In some embodiments, administration of the disclosed ADCs stimulates Type I IFN-dependent anti-tumor activity. As used herein, an “activated macrophage” is synonymous with a “polarized macrophage.”

Provided herein are ADC compounds comprising an antibody or antigen-binding fragment thereof (Ab) which targets a tumor cell, a drug moiety (D), and a linker moiety (L) that covalently attaches Ab to D. In certain aspects, the antibody or antigen-binding fragment is able to bind to a tumor-associated antigen (e.g., PSMA) with high specificity and high affinity. In certain embodiments, the antibody or antigen-binding fragment is internalized into a target cell upon binding, e.g., into a degradative compartment in the cell. In some embodiments, ADCs internalize upon binding to a target cell, undergo degradation, and release the drug moiety. The drug moiety may be released from the antibody and/or the linker moiety of the ADC by enzymatic action, hydrolysis oxidation, or any other mechanism.

In some embodiments, target cells bound by the ADC are phagocytosed by a myeloid cell, e.g., a macrophage or dendritic cell. In some embodiments, upon phagocytosis the ADCs undergo degradation and release the drug moiety. In some embodiments, the drug moiety is released in the phagolysosome of the myeloid cell (e.g., a macrophage or dendritic cell). The drug moiety may be released from the antibody and/or the linker moiety of the ADC by enzymatic action, hydrolysis, oxidation, or any other mechanism.

An exemplary ADC has Formula I:

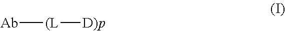

wherein Ab=antibody moiety (i.e., antibody or antigen-binding fragment), L=linker moiety, D=drug moiety, and p=the number of drug moieties per antibody moiety.

In some embodiments, an antibody-drug conjugate disclosed herein comprises an anti-PSMA antibody or antigen-binding fragment. In some embodiments, the anti-PSMA antibody or antigen-binding fragment comprises a heavy chain having an amino acid sequence selected from SEQ ID NOs: 47-60 listed in Table 8, supra and/or comprising a set of CDRs and/or a variable domain from the amino acid sequences in Table 8. In some embodiments, the anti-PSMA antibody or antigen-binding fragment comprises a light chain having an amino acid sequence selected from SEQ ID NOs: 61-66 listed in Table 8, infra and/or comprising a set of CDRs and/or a variable domain from the amino acid sequences in Table 8.

In some embodiments, the anti-PSMA antibody or antigen-binding fragment comprises one or more consensus CDR sequences of Table 1 in combination with one or more CDR sequences of Table 3, e.g., by selecting a HC CDR2, LC CDR1, and/or LCDR2 sequence of Table 1 and a HC CDR1, HC CDR2, HC CDR3, LC CDR1, LC CDR2, and/or LC CDR3 of Table 3 to describe an antibody by its three heavy chain and three light chain CDR sequences.

In some embodiments, the anti-PSMA antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system. In some embodiments, the anti-PSMA antibody or antigen-binding fragment comprises three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

In some embodiments, the anti-PSMA antibody or antigen-binding fragment comprises a heavy chain variable region comprising an amino acid sequence of SEQ ID NO: 14, and a light chain variable region comprising an amino acid sequence of SEQ ID NO: 19.

To accomplish site-specific conjugation of linkers and/or drug moieties to an antibody or antigen-binding fragment thereof, in some embodiments, a linker comprising a thiol-reactive group is used to generate a conjugated antibody or antigen-binding fragment, e.g., by reacting with the antibody or antigen-binding fragment at a cysteine residue. In some embodiments, the cysteine residue is at amino acid position 80 on the light chain. In some embodiments, the cysteine residue is at amino acid position 118 on the heavy chain. Methods to accomplish site-specific conjugation of linkers and/or drug moieties to an antibody or antigen-binding fragment for the production of ADCs are known in the art and disclosed in PCT application WO 2016/205618, herein incorporated by reference in its entirety.

### Drug Loading

Drug loading is represented by p and is also referred to herein as the drug-to-antibody ratio (DAR). In some embodiments, drug loading may range from 1 to 20 (i.e., 1 to 20 copies of the linker-drug attached to each antibody moiety), e.g., 1 to 12 drug moieties per antibody moiety. In some embodiments, p is an integer from 1 to 12. In some embodiments, p is an integer from 1 to 8. In some embodiments, p is an integer from 1 to 12, 1 to 11, 1 to 10, 1 to 9, 1 to 8, 1 to 7, 1 to 6, 1 to 5, 1 to 4, 1 to 3, or 1 to 2. In some embodiments, p is an integer from 2 to 12, 2 to 11, 2 to 10, 2 to 9, 2 to 8, 2 to 7, 2 to 6, 2 to 5, 2 to 4, or 2 to 3. In some embodiments, p is an integer from 2 to 11. In some embodiments, p is 1, 2, 3, 4, 5, 6, 7, 8, 9, 10, 11, or 12. In some embodiments, p is 2. In some embodiments, p is 4. In some embodiments, p is 7. In some embodiments, p is 8. In some embodiments, p is 11. In some embodiments, drug loading may be expressed as an average loading in a population of antibodies, e.g., an average loading of about 1-12, e.g., about 2-11. In some embodiments, the average drug loading in a population of antibodies is about 2 to about 8. In some embodiments, the average drug loading in a population of antibodies is about 2, about 4, or about 8.

Drug loading may be limited by the number of attachment sites on the antibody moiety. In some embodiments, the linker moiety (L) of the ADC attaches to the antibody moiety through a chemically active group on one or more amino acid residues on the antibody moiety. For example, the linker may be attached to the antibody moiety via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody moiety, or it can be introduced into the antibody moiety, e.g., by DNA recombinant technology (e.g., by introducing a cysteine or lysine residue into the amino acid sequence) or by protein biochemistry (e.g., by reduction, pH adjustment, or hydrolysis). In some embodiments, the linker is attached to the antibody moiety via a cysteine residue. In some embodiments, the linker is attached to the antibody moiety via a lysine residue.

In some embodiments, the number of drug moieties that can be conjugated to an antibody moiety is limited by the number of free cysteine residues. For example, where the attachment is a cysteine thiol group, an antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies exist as disulfide bridges. Over-attachment of linker-toxin to an antibody may destabilize the antibody by reducing the cysteine residues available to form disulfide bridges. Therefore, an optimal drug:antibody ratio should increase potency of the ADC (by increasing the number of attached drug moieties per antibody) without destabilizing the antibody moiety. In some embodiments, an optimal ratio may be about 2, 4, 7, or 11.

In some embodiments, one or more site-specific conjugation technologies are used to attach an ADC, e.g., to produce a homogeneous ADC product with a defined drug loading, i.e., a defined drug-to-antibody ratio (DAR). In some embodiments, free cysteine residues can be generated in the light chain or heavy chain of antibodies for site-specific conjugation via Residue-SPEcific Conjugation Technology (RESPECT). Exemplary protocols for the generation of RESPECT-formatted antibodies are described in Albone et al. (2017) Cancer Biol. Ther. 18(5):347-57, and in Intl. Pub. Nos. WO/2016205618 and WO/2017106643, each of which is incorporated herein by reference for methods of performing site-specific conjugation. In some embodiments, an ADC is produced using site-specific conjugation to covalently attach an antibody moiety to a drug moiety via a linker (e.g., a linker-payload conjugate disclosed herein). In some embodiments, site-specific conjugation is used to target a DAR of about 2 for ADCs or compositions comprising a compound disclosed herein, e.g., a compound of Formula (III), Formula (IV), or Table 14, e.g., Compound 1.

In some embodiments, a linker attached to an antibody moiety through a Mal or MC moiety may provide a ratio of about 2, 4, 7, or 11. In some embodiments, an ADC comprising MC-Val-Ala-pAB-Compound 1 joined to an anti-PSMA antibody as disclosed herein has a ratio of about 2, 4, 7, or 11. In some embodiments, an ADC comprising MC-Val-Ala-pABC-MEC-Compound 1 joined to an anti-PSMA antibody as disclosed herein has a ratio of about 2, 4, 7, or 11. In some embodiments, an ADC comprising MC-Val-Ala-pABC-Unit 8-Compound 1 joined to an anti-PSMA antibody as disclosed herein has a ratio of about 2, 4, 7, or 11. In some embodiments, an ADC comprising MC-Val-Ala-pABC-Unit 9-Compound 1 joined to an anti-PSMA antibody as disclosed herein has a ratio of about 2, 4, 7, or 11. In some embodiments, an ADC comprising MC-Val-Ala-pABC-Unit 11-Compound 1 joined to an anti-PSMA antibody as disclosed herein has a ratio of about 2, 4, 7, or 11.

In some embodiments, an antibody moiety is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which preferentially reduces the interchain disulfide bonds which link the light chain and heavy chain (one pair per H-L pairing) and the two heavy chains in the hinge region (two pairs per H-H pairing in the case of human IgG1) while leaving the intrachain disulfide bonds intact. See, e.g., Stefano et al. (2013) Methods Mol. Biol. 1045:145-71. In some embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device). See, e.g., U.S. Publ. No. 20140069822. In certain embodiments, an antibody is subjected to denaturing conditions to reveal reactive nucleophilic groups on amino acid residues, such as lysine or cysteine.

The drug loading of an ADC may be controlled in different ways, e.g., by: (i) limiting the molar excess of drug-linker intermediate or linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine or lysine residues is modified for control of the number and/or position of linker-drug attachments.

In some embodiments, free cysteine residues are introduced into the amino acid sequence of the antibody moiety. For example, cysteine engineered antibodies can be prepared wherein one or more amino acids of a parent antibody are replaced with a cysteine amino acid. Any form of antibody may be so engineered, i.e., mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art. See, e.g., the methods described in WO2006/034488. These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula I include, but are not limited to, antibodies that have 1, 2, 3, or 4 engineered cysteine amino acids. See Lyon et al. (2012) Methods Enzymol. 502:123-38. In some embodiments, one or more free cysteine residues are already present in an antibody moiety, without the use of engineering, in which case the existing free cysteine residues may be used to conjugate the antibody moiety to a drug moiety.

Where more than one nucleophilic group reacts with a drug-linker intermediate or a linker moiety reagent followed by a drug moiety reagent, in a reaction mixture comprising multiple copies of the antibody moiety and linker moiety, then the resulting product can be a mixture of ADC compounds with a distribution of one or more drug moieties attached to each copy of the antibody moiety in the mixture. In some embodiments, the drug loading in a mixture of ADCs resulting from a conjugation reaction ranges from 1 to 12 drug moieties attached per antibody moiety. The average number of drug moieties per antibody moiety (i.e., the average drug loading, or average p) may be calculated by any conventional method known in the art, e.g., by mass spectrometry (e.g., reverse-phase LC-MS), and/or high-performance liquid chromatography (e.g., HPLC). In some embodiments, the average number of drug moieties per antibody moiety is determined by hydrophobic interaction chromatography-high performance liquid chromatography (HIC-HPLC). In some embodiments, the average number of drug moieties per antibody moiety is determined by reverse-phase liquid chromatography-mass spectrometry (LC-MS). In some embodiments, the average number of drug moieties per antibody moiety is from about 1 to about 11; from about 1 to about 8; from about 1 to about 7; from about 1 to about 4; or from about 1 to about 2. In some embodiments, the average number of drug moieties per antibody moiety is about 2. In some embodiments, the average number of drug moieties per antibody moiety is about 4. In some embodiments, the average number of drug moieties per antibody moiety is about 7. In some embodiments, the average number of drug moieties per antibody moiety is about 11.

Individual ADC compounds having particular DAR ratios, or “species,” may be identified in the mixture, e.g., by mass spectroscopy and separated, e.g., by ultra-performance liquid chromatography (UPLC) or HPLC, e.g., hydrophobic interaction chromatography (HIC-HPLC). In certain embodiments, a homogeneous or nearly homogenous ADC with a single loading value may be isolated from the conjugation mixture, e.g., by electrophoresis or chromatography.

The present disclosure includes methods of producing the described ADCs. Briefly, the ADCs comprise an antibody or antigen-binding fragment as the antibody moiety, a drug moiety, and a linker that joins the drug moiety and the antibody moiety. In some embodiments, the ADCs can be prepared using a linker having reactive functionalities for covalently attaching to the drug moiety and to the antibody moiety. For example, in some embodiments, a cysteine thiol of an antibody moiety can form a bond with a reactive functional group of a linker or a drug-linker intermediate (e.g., a Mal or MC moiety), the linker or linker intermediate comprising a reactive group that can be conjugated to a functional agent (e.g., a cleavable peptide comprising Compound 1).

In some embodiments, an ADC is produced by contacting an antibody or antigen-binding fragment with a linker and a drug moiety in a sequential manner, such that the antibody moiety is covalently linked to the linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody moiety with a linker-drug conjugate, or a salt thereof, pre-formed by reacting a linker with a drug moiety. The pre-formed linker-drug conjugate may or may not be subjected to a purification step prior to contacting the antibody moiety. In other embodiments, the antibody moiety contacts the linker and the drug moiety in one reaction mixture, allowing simultaneous formation of the covalent bonds between the antibody moiety and the linker, and between the linker and the drug moiety. In some embodiments, an ADC is produced by reacting an antibody moiety with a linker joined to a drug moiety, such as LP3, or a salt thereof, under conditions that allow conjugation. In some embodiments, an ADC is produced by reacting an antibody moiety with a linker joined to a drug moiety, such as LP1, or a salt thereof, under conditions that allow conjugation. In some embodiments, an ADC is produced by reacting an antibody moiety with a linker joined to a drug moiety, such as LP2, or a salt thereof, under conditions that allow conjugation. In some embodiments, an ADC is produced by reacting an antibody moiety with a linker joined to a drug moiety, such as LP16, or a salt thereof, under conditions that allow conjugation. In some embodiments, an ADC is produced by reacting an antibody moiety with a linker joined to a drug moiety, such as LP20, or a salt thereof, under conditions that allow conjugation. In some embodiments, an ADC is produced by reacting an antibody moiety with a linker joined to a drug moiety, such as LP26, or a salt thereof, under conditions that allow conjugation. In some embodiments, an ADC is produced by reacting an antibody moiety with a linker joined to a drug moiety, such as LP28, or a salt thereof, under conditions that allow conjugation. The conditions that allow conjugation may involve any biochemical methods known in the art for conjugating an ADC. These conditions include, but are not limited to, incubation at room temperature in a suitable buffer (e.g., 1×DBPS, 0.1 M Tris-Glycine at pH 7.4, 10% propylene glycol:90% 1×DPBS, or 1×DPBS, 2 mM EDTA). The conjugation conditions may or may not include the presence of an enzyme (e.g., transglutaminase).

The ADCs prepared according to the methods described above may be subjected to one or more purification steps. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, desalting chromatography, or any combination thereof.

### Therapeutic Uses

Disclosed herein are methods of using the disclosed antibodies and/or ADCs in treating a subject for a disorder, e.g., an oncologic disorder. Antibodies and/or ADCs may be administered alone or in combination with one or more additional therapeutic agent(s), and may be administered in any pharmaceutically acceptable formulation, dosage, and/or dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an antibody or ADC exerts a cytostatic and/or cytotoxic effect on a cell are known. For example, the cytotoxic activity of an antibody or ADC can be measured by: exposing mammalian cells expressing a target protein of the antibody or ADC (e.g., PSMA) in a cell culture medium; co-culturing the cells with immune cells (e.g., myeloid cells such as macrophages) for a period of time, e.g., from about 6 hours to about 5 days; and measuring cell viability. Cell-based in vitro assays may also be used to measure viability (proliferation), cytotoxicity, growth inhibition, and induction of apoptosis (caspase activation) of the antibody or ADC.

In some embodiments, to determine cytotoxicity, phagocytosis, necrosis, or apoptosis (programmed cell death) may be measured. Phagocytosis may be observed using flow cytometry or microscopy. Necrosis is typically accompanied by increased permeability of the plasma membrane, swelling of the cell, and rupture of the plasma membrane. Apoptosis is typically characterized by membrane blebbing, condensation of cytoplasm, and the activation of endogenous endonucleases. Determination of any of these effects on cancer cells indicates that an antibody or ADC is useful in the treatment of cancers.

Cell viability may be measured, e.g., by determining in a cell the uptake of a dye such as neutral red, trypan blue, Crystal Violet, or ALAMAR™ blue. See, e.g., Page et al. (1993) Intl. J. Oncology 3:473-6. In such an assay, the cells are incubated in media containing the dye, the cells are washed, and the remaining dye, reflecting cellular uptake of the dye, is measured spectrophotometrically. In certain embodiments, in vitro potency of prepared antibodies or ADCs is assessed using a Crystal Violet assay. Crystal Violet is a triarylmethane dye that accumulates in the nucleus of viable cells. In this assay, cells are exposed to the antibodies or ADCs or control agents for a defined period of time, after which cells are stained with Crystal Violet, washed copiously with water, then solubilized with 1% SDS and read spectrophotometrically. The protein-binding dye sulforhodamine B (SRB) can also be used to measure cytoxicity. See, e.g., Skehan et al. (1990) J. Natl. Cancer Inst. 82:1107-12.

Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) No. 2, pp. 34-37 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as acridine orange or ethidium bromide). A method for measuring apoptotic cell number has been described by Duke and Cohen, Current Protocols in Immunology. See Coligan et al., eds. (1992) pp. 3.17.1-3.17.16. Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

In some embodiments, the present disclosure provides a method of killing, or inhibiting or modulating the growth of, a cancer cell or tissue by agonizing the STING pathway and targeting that agonism to particular cells, e.g., cancer cells expressing PSMA (e.g., increased expression level of PSMA relative to non-cancer cells). In some embodiments, agonizing the STING pathway boosts antitumor immunity, e.g., by activating myeloid cells (e.g., macrophages or dendritic cells), cytotoxic T cells, and/or type 1 T helper cell (Th1)-biased responses. The method may be used with any subject where boosting antitumor immunity provides a therapeutic benefit. The antitumor immune response may target a cancer cell regardless of PSMA expression levels.

In various embodiments, the disclosed antibodies and/or ADCs may be administered to affect any cell or tissue that expresses PSMA, such as a PSMA-expressing cancer cell or tissue. An exemplary embodiment comprises a method of killing a cell via systemic delivery of a STING agonist, e.g., a compound of Formula (III), Formula (IV), or Table 14, e.g., Compound 1, in an anti-PSMA ADC. The method may be used with any cell or tissue that expresses PSMA, such as a cancerous cell or metastatic lesion. In some embodiments, the PSMA-expressing cancer is prostate cancer. In some embodiments, the prostate cancer is advanced prostate cancer. In some embodiments, the prostate cancer is metastatic castration-resistant prostate cancer. Non-limiting examples of PSMA-expressing cells include 22RV1, LNCaP, and C4-2 cells, and cells comprising a recombinant nucleic acid encoding PSMA or a portion thereof. Without being bound by theory, the anti-PSMA antibodies and ADCs disclosed herein may be particularly effective at treating PSMA-expressing cancers by targeting PSMA-expressing cells for immune clearance, by activating type I interferons and other inflammatory cytokines (e.g., IFN-β, TNFα, CXCL10, and/or IL-6), and/or by delivering a drug payload (e.g., Compound 1) to cells.

In some embodiments, an ADC may be used to deliver a drug payload (e.g., Compound 1) to cells, wherein the drug payload activates the STING pathway. Without being bound by theory, STING pathway activation may result in the activation of type I interferons and other inflammatory cytokines (e.g., IFN-β, TNFα, CXCL10, and/or IL-6). In some embodiments, administration of the anti-PSMA ADCs disclosed herein increases expression and/or secretion of IFN-β. In some embodiments, administration of the anti-PSMA ADCs disclosed herein increases expression and/or secretion of TNFα. In some embodiments, administration of the anti-PSMA ADCs disclosed herein increases expression and/or secretion of CXCL10. In some embodiments, administration of the anti-PSMA ADCs disclosed herein increases expression and/or secretion of IL-6. The activation of type I interferons and other inflammatory cytokines may stimulate antitumor immune response by activating dendritic cells and proinflammatory (M1) macrophages, by promoting the generation of cytotoxic T cell responses, and/or by promoting the generation of type 1 T helper cell (Th1)-biased responses. The cancer cell or tumor may then be targeted for killing by these activated immune cells. Activated dendritic cells and proinflammatory (M1) macrophages may also produce type I interferons and other inflammatory cytokines or chemokines, thus enhancing the antitumor inflammatory response. Non-limiting examples of macrophage cells include J774A.1, THP-1, bone marrow-derived macrophages (BMDM), human monocyte derived macrophages (HMDM), and peripheral blood mononuclear cells (PBMC).

In some embodiments, the anti-PSMA antibodies and antigen-binding fragments disclosed herein provide for stable systemic delivery of a STING agonist to a cancer cell or tissue. In some embodiments, the STING agonist is Compound 1. In some embodiments, the cancer cell or tissue expresses PSMA. In some embodiments, the cancer cell or tissue is a prostate cancer. In some embodiments, the prostate cancer is advanced prostate cancer. In some embodiments, the prostate cancer is metastatic castration-resistant prostate cancer.

Exemplary methods disclosed herein include the steps of contacting a cell with an antibody and/or ADC as described herein (e.g., by administering the antibody and/or ADC to a subject by a suitable route of administration), in an effective amount, e.g., an amount sufficient to stimulate STING activity. The method can be used on cells in culture, e.g., in vitro, ex vivo, or in situ. For example, cells that express PSMA (e.g., cells collected by biopsy of a tumor or metastatic lesion; cells from an established cancer cell line; or recombinant cells), can be cultured in vitro in culture medium and the contacting step can be affected by adding the antibody and/or ADC to the culture medium. In cells co-cultured with immune cells (e.g., macrophages or dendritic cells), the method will result in killing of cells expressing PSMA, including in particular tumor cells expressing PSMA. Alternatively, the antibody and/or ADC can be administered to a subject by any suitable administration route (e.g., intravenous, subcutaneous, or direct contact with a tumor tissue) to have an effect in vivo.

The in vivo effect of a disclosed antibody and/or ADC can be evaluated in a suitable animal model. For example, xenogenic cancer models can be used, wherein cancer explants or passaged xenograft cells or tissues are introduced into immune compromised animals, such as nude or SCID mice. See, e.g., Klein et al. (1997) Nature Med. 3:402-8. Efficacy may be predicted using assays that measure inhibition of tumor formation, tumor regression or metastasis, and the like. In some embodiments, the anti-PSMA antibodies and ADCs disclosed herein are more efficacious at inhibiting tumor growth compared to xenograft-bearing mice treated with alternate treatments.

Assays that measure the expression of PSMA and/or cytokines may also be used. Any method for measuring PSMA and/or cytokine expression known in the art may be used, including ELISA (enzyme-linked immunosorbent assay), q-PCR (quantitative polymerase chain reaction), Meso Scale Discovery V-PLEX Cytokine Panel, immunohistochemistry, RNA-seq (RNA-sequencing), Western blot, and flow cytometry. The expression of PSMA may be determined in cancer cells isolated from a subject. In some embodiments, the expression of PSMA is determined prior to administration of an antibody and/or ADC as disclosed herein. In some embodiments, the expression of PSMA is elevated relative to the expression of PSMA in non-cancerous and/or wild-type tissue or cells. In some embodiments, expression of type I interferons and other inflammatory cytokines (e.g., IFN-β, TNFα, CXCL10, IL-6) is measured. The expression of these cytokines in the tumors of treated subjects may provide an indication of the stimulation of antitumor immune response in response to a treatment.

In various embodiments, provided herein are methods of treating PSMA-expressing cancer. The antibodies and ADCs disclosed herein can be administered to a non-human mammal or human subject for any therapeutic purposes and via any suitable administration route. The therapeutic methods may entail administering to a mammal having a tumor, e.g., a tumor expressing PSMA, a biologically effective amount of an antibody disclosed herein or an ADC comprising a selected chemotherapeutic agent (e.g., Compound 1) linked to that antibody.

In some embodiments, a method of treating a patient having or at risk of having a cancer that expresses PSMA is provided, comprising administering to the patient a therapeutically effective amount of an antibody and/or ADC of the present disclosure. In some embodiments, the patient is non-responsive or poorly responsive to treatment with a drug moiety (e.g., Compound 1) when administered alone, and the patient is administered an antibody or ADC disclosed herein. In other embodiments, the patient is intolerant to treatment with a drug moiety (e.g., Compound 1) when administered alone. For instance, to treat a cancer, a patient may require doses of Compound 1 that lead to systemic toxicity, which are overcome by targeted delivery of the antibodies and/or ADCs disclosed herein to a PSMA-expressing cancer, thereby reducing off-target killing. In some embodiments, the patient has a cancer that is inaccessible to local injection of a drug moiety (e.g., Compound 1).

In various embodiments, the methods disclosed herein treat prostate cancer.

The antibodies and/or ADCs of the present disclosure may be administered to a non-human mammal expressing PSMA for veterinary purposes or as an animal model of human disease. Regarding the latter, such animal models may be useful for evaluating the therapeutic efficacy of the disclosed antibodies and ADCs (e.g., testing of dosages and time courses of administration).

In some embodiments, the efficacy of an antibody or ADC may be evaluated by contacting a tumor sample from a subject with the antibody or ADC and evaluating tumor growth rate or volume. In some embodiments, when an antibody or ADC has been determined to be effective, it may be administered to the subject. In some embodiments, the efficacy of an antibody or ADC may be evaluated by contacting a subject with the antibody or ADC and monitoring tumor growth rate or volume. In some embodiments, the efficacy of an antibody or ADC may be evaluated by contacting a subject with the antibody or ADC and monitoring expression of type I interferons and other inflammatory cytokines (e.g., IFN-β, TNFα, CXCL10, IL-6).

The antibodies and ADCs disclosed herein may be administered at a suitable dosage to a patient in need thereof. Dosages and administration protocols for the treatment of cancers using the foregoing methods will vary with the method and the target cancer, and will generally depend on a number of other factors appreciated in the art.

In various embodiments, treatment involves single bolus or repeated administration of an antibody or ADC preparation via an acceptable route of administration.

The above therapeutic approaches can also be combined with any one of a wide variety of additional surgical, chemotherapy, or radiation therapy regimens. In some embodiments, the above therapeutic approaches are combined with a cancer immunotherapy, e.g., immune checkpoint therapy (e.g., PD-1/PD-L1 inhibitors and CTLA4 inhibitors) or adoptive T cell (ATC) therapy (e.g., chimeric antigen receptor (CAR) T cells).

Further provided herein are therapeutic uses of the disclosed antibodies and/or ADCs. An exemplary embodiment is the use of an antibody and/or ADC in the treatment of a PSMA-expressing cancer, such as prostate cancer. Methods for identifying subjects having cancers that express PSMA are known in the art and may be used to identify suitable patients for treatment with a disclosed antibody or ADC.

Another exemplary embodiment is the use of an ADC or an antibody or antigen-binding fragment as disclosed herein in the manufacture of a medicament for the treatment of a PSMA-expressing cancer, such as prostate cancer.

### Pharmaceutical Compositions and Formulations

An antibody or ADC used in the practice of the foregoing methods may be formulated into a pharmaceutical composition suitable for administration to a subject, e.g., a human subject. In some embodiments, the pharmaceutical composition comprises the antibody and/or ADC and a pharmaceutically acceptable carrier suitable for the desired delivery method. Suitable carriers include any material that, when combined with an antibody or ADC disclosed herein, allows that antibody or ADC to retain its antitumor function and is generally non-reactive with the patient's immune system. Pharmaceutically acceptable carriers may include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives, or buffers, which enhance the shelf life or effectiveness of the ADC.

The pharmaceutical compositions described herein may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The preferred form depends on the intended mode of administration and therapeutic application.

Pharmaceutical compositions may be solubilized and administered via any route capable of delivering the composition to the tumor site. Potentially effective routes of administration include, but are not limited to, intravenous, parenteral, intraperitoneal, intramuscular, intratumor, intradermal, intraorgan, orthotopic, and the like. Pharmaceutical compositions can be lyophilized and stored as sterile powders, preferably under vacuum, and then reconstituted in bacteriostatic water (containing, for example, benzyl alcohol preservative) or in sterile water prior to injection. Administration can be either systemic or local. Pharmaceutical compositions may comprise an antibody and/or ADC or a pharmaceutically acceptable salt thereof, e.g., a mesylate salt.

In various embodiments, kits for use in the laboratory and the therapeutic applications described herein are within the scope of the present disclosure. Such kits may comprise an antibody or ADC disclosed herein and a carrier, package, or container. The carrier, package, or container may be compartmentalized to receive one or more containers such as vials, tubes, and the like, each of the container(s) comprising one of the separate elements to be used in a method disclosed herein, and/or a label or insert comprising instructions for use, such as a use described herein. Kits may further comprise one or more other containers associated therewith that comprise materials desirable from a commercial and user standpoint, including buffers, diluents, filters, needles, syringes, carrier, package, container, vial and/or tube labels listing contents and/or instructions for use, and package inserts with instructions for use.

A label may be present on or with the container to indicate that the composition is used for a specific therapy or non-therapeutic application, such as a prognostic, prophylactic, diagnostic, or laboratory application. A label may also indicate directions for either in vivo or in vitro use, such as those described herein. Directions and/or other information may also be included on an insert(s) or label(s) which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the composition is used for diagnosing or treating a condition, such as a cancer as described herein.

### Exemplary Embodiments of the Disclosure

In various embodiments, the present disclosure provides novel linker-drug conjugates that are capable of being conjugated to an antibody in an antibody-drug conjugate.

In some embodiments, the linker-drug conjugate comprises MC-Val-Ala-pABC-Unit 8 moiety and a compound of Formula (III). In some embodiments, the linker-drug conjugate comprises MC-Val-Ala-pABC-Unit 8 moiety and a compound of Formula (IV). In some embodiments, the linker-drug conjugate comprises MC-Val-Ala-pABC-Unit 8 moiety and a compound selected from a compound of Table 14.

In some embodiments, the linker-drug conjugate comprises MC-Val-Ala-pABC-Unit 8-Compound 1. In some embodiments, the linker-drug conjugate comprises LP16.

In some embodiments, the linker-drug conjugate comprises MC-Val-Ala-pABC-Unit 9 moiety and a compound of Formula (III). In some embodiments, the linker-drug conjugate comprises MC-Val-Ala-pABC-Unit 9 moiety and a compound of Formula (IV). In some embodiments, the linker-drug conjugate comprises MC-Val-Ala-pABC-Unit 9 moiety and a compound selected from a compound of Table 14.

In some embodiments, the linker-drug conjugate comprises MC-Val-Ala-pABC-Unit 9-Compound 1. In some embodiments, the linker-drug conjugate comprises LP20.

In some embodiments, the linker-drug conjugate comprises Mal-Formula (II)-Val-Ala-pAB-Unit 11 moiety and a compound of Formula (III). In some embodiments, the linker-drug conjugate comprises Mal-Formula (II)-Val-Ala-pAB-Unit 11 moiety and a compound of Formula (IV). In some embodiments, the linker-drug conjugate comprises Mal-Formula (II)-Val-Ala-pAB-Unit 11 moiety and a compound selected from a compound of Table 14.

In some embodiments, the linker-drug conjugate comprises Mal-Formula (II)-Val-Ala-pAB-Unit 11-Compound 1. In some embodiments, the linker-drug conjugate comprises LP26.

In some embodiments, the linker-drug conjugate comprises MC-Val-Ala-pABC-Unit 11 moiety and a compound of Formula (III). In some embodiments, the linker-drug conjugate comprises MC-Val-Ala-pABC-Unit 11 moiety and a compound of Formula (IV). In some embodiments, the linker-drug conjugate comprises MC-Val-Ala-pABC-Unit 11 moiety and a compound selected from a compound of Table 14.

In some embodiments, the linker-drug conjugate comprises MC-Val-Ala-pABC-Unit 11-Compound 1. In some embodiments, the linker-drug conjugate comprises LP28.

In various embodiments, the present disclosure provides novel antibody-drug conjugates capable of specifically binding PSMA.

In some embodiments, the antibody-drug conjugate comprises any anti-PSMA antibody or antigen-binding fragment disclosed herein, a linker comprising MC-Val-Ala-pABC-Unit 8 moiety, and a drug moiety comprising a compound of Formula (III). In some embodiments, the antibody-drug conjugate comprises any anti-PSMA antibody or antigen-binding fragment disclosed herein, a linker comprising MC-Val-Ala-pABC-Unit 8 moiety, and a drug moiety comprising a compound of Formula (IV). In some embodiments, the antibody-drug conjugate comprises any anti-PSMA antibody or antigen-binding fragment disclosed herein, a linker comprising MC-Val-Ala-pABC-Unit 8 moiety, and a drug moiety comprising a compound selected from a compound of Table 14.

In some embodiments, the antibody-drug conjugate comprises any anti-PSMA antibody or antigen-binding fragment disclosed herein and a linker-drug conjugate comprising MC-Val-Ala-pABC-Unit 8-Compound 1. In some embodiments, the antibody-drug conjugate comprises any anti-PSMA antibody or antigen-binding fragment disclosed herein conjugated to LP16.

In some embodiments, the antibody-drug conjugate comprises any anti-PSMA antibody or antigen-binding fragment disclosed herein, a linker comprising MC-Val-Ala-pABC-Unit 9 moiety, and a drug moiety comprising a compound of Formula (III). In some embodiments, the antibody-drug conjugate comprises any anti-PSMA antibody or antigen-binding fragment disclosed herein, a linker comprising MC-Val-Ala-pABC-Unit 9 moiety, and a drug moiety comprising a compound of Formula (IV). In some embodiments, the antibody-drug conjugate comprises any anti-PSMA antibody or antigen-binding fragment disclosed herein, a linker comprising MC-Val-Ala-pABC-Unit 9 moiety, and a drug moiety comprising a compound selected from a compound of Table 14.

In some embodiments, the antibody-drug conjugate comprises any anti-PSMA antibody or antigen-binding fragment disclosed herein and a linker-drug conjugate comprising MC-Val-Ala-pABC-Unit 9-Compound 1. In some embodiments, the antibody-drug conjugate comprises any anti-PSMA antibody or antigen-binding fragment disclosed herein conjugated to LP20.

In some embodiments, the antibody-drug conjugate comprises any anti-PSMA antibody or antigen-binding fragment disclosed herein, a linker comprising Mal-Formula (II)-Val-Ala-pAB-Unit 11 moiety, and a drug moiety comprising a compound of Formula (III). In some embodiments, the antibody-drug conjugate comprises any anti-PSMA antibody or antigen-binding fragment disclosed herein, a linker comprising Mal-Formula (II)-Val-Ala-pAB-Unit 11 moiety, and a drug moiety comprising a compound of Formula (IV). In some embodiments, the antibody-drug conjugate comprises any anti-PSMA antibody or antigen-binding fragment disclosed herein, a linker comprising Mal-Formula (II)-Val-Ala-pAB-Unit 11 moiety, and a drug moiety comprising a compound selected from a compound of Table 14.

In some embodiments, the antibody-drug conjugate comprises any anti-PSMA antibody or antigen-binding fragment disclosed herein and a linker-drug conjugate comprising Mal-Formula (II)-Val-Ala-pAB-Unit 11-Compound 1. In some embodiments, the antibody-drug conjugate comprises any anti-PSMA antibody or antigen-binding fragment disclosed herein conjugated to LP26.

In some embodiments, the antibody-drug conjugate comprises any anti-PSMA antibody or antigen-binding fragment disclosed herein, a linker comprising MC-Val-Ala-pABC-Unit 11 moiety, and a drug moiety comprising a compound of Formula (III). In some embodiments, the antibody-drug conjugate comprises any anti-PSMA antibody or antigen-binding fragment disclosed herein, a linker comprising MC-Val-Ala-pABC-Unit 11 moiety, and a drug moiety comprising a compound of Formula (IV). In some embodiments, the antibody-drug conjugate comprises any anti-PSMA antibody or antigen-binding fragment disclosed herein, a linker comprising MC-Val-Ala-pABC-Unit 11 moiety, and a drug moiety comprising a compound selected from a compound of Table 14.

In some embodiments, the antibody-drug conjugate comprises any anti-PSMA antibody or antigen-binding fragment disclosed herein and a linker-drug conjugate comprising MC-Val-Ala-pABC-Unit 11-Compound 1. In some embodiments, the antibody-drug conjugate comprises any anti-PSMA antibody or antigen-binding fragment disclosed herein conjugated to LP28. In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; any linker disclosed herein; and a drug moiety comprising a compound of Formula (III). In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; any linker disclosed herein; and a drug moiety comprising a compound of Formula (IV). In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; any linker disclosed herein; and a drug moiety comprising a compound selected from a compound of Table 14.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; any linker disclosed herein; and a drug moiety comprising a compound of Formula (III). In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; any linker disclosed herein; and a drug moiety comprising a compound of Formula (IV). In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; any linker disclosed herein; and a drug moiety comprising a compound selected from a compound of Table 14.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; any linker disclosed herein; and a drug moiety comprising a compound of Formula (III). In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; any linker disclosed herein; and a drug moiety comprising a compound of Formula (IV). In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; any linker disclosed herein; and a drug moiety comprising a compound selected from a compound of Table 14.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; any linker disclosed herein; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; any linker disclosed herein; and a drug moiety comprising Compound 1.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; any linker disclosed herein; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; a linker comprising MC-Val-Ala-pABC-Unit 8; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; and a linker-drug conjugate comprising LP16.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; a linker comprising MC-Val-Ala-pABC-Unit 8 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; and a linker-drug conjugate comprising LP16.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; a linker comprising MC-Val-Ala-pABC-Unit 8 moiety; and a drug moiety comprising Compound 1.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; and a linker-drug conjugate comprising LP16.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; a linker comprising MC-Val-Ala-pABC-Unit 9 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; and a linker-drug conjugate comprising LP20.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; a linker comprising MC-Val-Ala-pABC-Unit 9 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; and a linker-drug conjugate comprising LP20.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; a linker comprising MC-Val-Ala-pABC-Unit 9 moiety; and a drug moiety comprising Compound 1.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; and a linker-drug conjugate comprising LP20.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; a linker comprising Mal-Formula (II)-Val-Ala-pAB-Unit 11 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; and a linker-drug conjugate comprising LP26.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; a linker comprising Mal-Formula (II)-Val-Ala-pAB-Unit 11 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; and a linker-drug conjugate comprising LP26.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; a linker comprising Mal-Formula (II)-Val-Ala-pAB-Unit 11 moiety; and a drug moiety comprising Compound 1.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; and a linker-drug conjugate comprising LP26.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; a linker comprising MC-Val-Ala-pABC-Unit 11 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 21, heavy chain CDR2 (HCDR2) comprising SEQ ID NO: 22, heavy chain CDR3 (HCDR3) comprising SEQ ID NO: 27; light chain CDR1 (LCDR1) comprising SEQ ID NO: 32, light chain CDR2 (LCDR2) comprising SEQ ID NO: 35, and light chain CDR3 (LCDR3) comprising SEQ ID NO: 37, as defined by the Kabat numbering system; and a linker-drug conjugate comprising LP28.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; a linker comprising MC-Val-Ala-pABC-Unit 11 moiety; and a drug moiety comprising Compound 1.

In some embodiments, the antibody-drug conjugate comprises an anti-PSMA antibody or antigen-binding fragment thereof comprising three heavy chain CDRs and three light chain CDRs as follows: heavy chain CDR1 (HCDR1) comprising SEQ ID NO: 28, heavy chain CDR2 (HCDR2) comprising SEQ ID NO: 29, heavy chain CDR3 (HCDR3) comprising SEQ ID NO: 30; light chain CDR1 (LCDR1) comprising SEQ ID NO: 38, light chain CDR2 (LCDR2) comprising SEQ ID NO: 39, and light chain CDR3 (LCDR3) comprising SEQ ID NO: 37, as defined by the IMGT numbering system; and a linker-drug conjugate comprising LP28.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; a linker comprising MC-Val-Ala-pABC-Unit 11 moiety; and a drug moiety comprising Compound 1.

In some embodiments, the antibody-drug conjugate comprises an anti-PSMA antibody or antigen-binding fragment thereof comprising a heavy chain variable region comprising the amino acid sequence of SEQ ID NO: 14, and a light chain variable region comprising the amino acid sequence of SEQ ID NO: 19; and a linker-drug conjugate comprising LP28.

In some embodiments of antibody-drug conjugates disclosed herein, the antibody-drug conjugate comprises an anti-PSMA antibody or antigen-binding fragment thereof which comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; and a linker-drug conjugate comprising LP16, LP20, LP26, or LP28. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity). In some embodiments, without being bound by theory, benefits of using an antibody-drug conjugate comprising an anti-PSMA antibody or antigen-binding fragment thereof comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; and a linker-drug conjugate comprising LP16, LP20, LP26, or LP28, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity. Exemplary evidence of the superior benefits of such antibody-drug conjugates is shown in Examples 9, 12, 14, and 15.

In some embodiments of antibody-drug conjugates disclosed herein, the antibody-drug conjugate comprises an anti-PSMA antibody or antigen-binding fragment thereof which comprises three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and a linker-drug conjugate comprising LP16, LP20, LP26, or LP28. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity) compared to other anti-PSMA antibody-drug conjugates. In some embodiments, without being bound by theory, benefits of using an antibody-drug conjugate comprising an anti-PSMA antibody or antigen-binding fragment thereof comprises three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and a linker-drug conjugate comprising LP16, LP20, LP26, or LP28, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity. Exemplary evidence of the superior benefits of such antibody-drug conjugates is shown in Examples 9, 12, 14, and 15.

In some embodiments of antibody-drug conjugates disclosed herein, the antibody-drug conjugate comprises an anti-PSMA antibody or antigen-binding fragment thereof which comprises a heavy chain variable region comprising an amino acid sequence of SEQ ID NO: 14, and a light chain variable region comprising an amino acid sequence of SEQ ID NO: 19; and a linker-drug conjugate comprising LP16, LP20, LP26, or LP28. Without being bound by theory, one or more of these antibody-drug conjugates may demonstrate superior properties over other ADCs, e.g., those using other antibodies, linkers, and/or drugs, e.g., as compared to other ADCs disclosed herein (e.g., improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity). In some embodiments, without being bound by theory, benefits of using an antibody-drug conjugate comprising an anti-PSMA antibody or antigen-binding fragment thereof comprises a heavy chain variable region comprising an amino acid sequence of SEQ ID NO: 14, and a light chain variable region comprising an amino acid sequence of SEQ ID NO: 19; and a linker-drug conjugate comprising LP16, LP20, LP26, or LP28, may include improved conjugation stability, improved plasma stability, low ADC aggregation, on-target cytotoxicity, low off-target toxicity, pharmacokinetic and pharmacodynamics properties, formulatability, and toxicologic/immunologic profiles, stimulation of an anti-immune response in the tumor microenvironment, stimulation of increased phagocytosis of PSMA-expressing cells by myeloid cells (e.g., macrophages and/or dendritic cells), and in vivo anti-tumor activity. Exemplary evidence of the superior benefits of such antibody-drug conjugates is shown in Examples 9, 12, 14, and 15.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### Examples

### Example 1. Linker-Payload Synthesis

In the following illustrative examples, unless stated otherwise:
(i) temperatures are given in degrees Celsius (° C.);
(ii) organic solutions were dried over anhydrous sodium sulfate or magnesium sulfate;
(iii) evaporation of organic solvent was carried out using a rotary evaporator under reduced pressure (0-1000 mbar) with a bath temperature of up to 60° C.;
(iv) column chromatography means flash chromatography on silica gel or pre-packed silica gel cartridges (12 g, 24 g, 40 g, etc.);
(v) thin layer chromatography (TLC) was carried out on silica gel plates;
(vi) normal and reverse phase flash column chromatography were carried out using either Teledyne ISCO CombiFlash® systems or Biotage® Flash Columns, were used according to the manufacturers' instructions, and obtained from 4700 Superior Street, Lincoln NE 68504, USA or Biotage AB Box 8 751 03 Uppsala Sweden, respectively;
(vii) Prep-TLC means preparative TLC plates used in purification;
(viii) in general, the course of reactions was followed by TLC or liquid chromatography/mass spectroscopy (LC/MS) and reaction times are given for illustration only;
(ix) final products have satisfactory proton nuclear magnetic resonance (NMR) spectra and/or mass spectra data;
(x) preparations were repeated if more material was required;
(xi) when given, ¹H NMR data are in the form of delta values for major diagnostic protons, given in part per million (ppm) relative to tetramethylsilane (TMS, 0 ppm) as an internal standard. Residual solvent peaks can also be used as the internal standard. The coupling constants are reported in the unit of Hertz (Hz). Abbreviations for splitting patterns are as follows: s: singlet; d: doublet; t: triplet; m: multiplet; and brs: broad singlet;
(xii) in the event that the nomenclature assigned to a given compound does not correspond to the compound structure depicted herein, the structure will control; and
(xiii) Prep-HPLC means preparative high-performance liquid chromatography, referring to purification using reverse phase HPLC columns listed below, and used according to the manufacturer's instructions.

### Abbreviations

The following abbreviations may be used throughout the examples.
Boc: tert-butyloxycarbonyl
AcOH: Acetic acid
Cbz: Benzyloxycarbonyl
CSA: Camphorsulfonic acid
DCC: N,N′-Dicyclohexylcarbodiimide
DCM: Dichloromethane
DIBAL-H: Diisobutylaluminium hydride
DIEA: N,N-Diisopropylethylamine
DIPEA: N,N-Diisopropylethylamine
DME: 1,2-Dimethoxyethane
DMF: N,N-Dimethylformamide
DMT-MM: 4-(4,6-dimethoxy-1,3,5-triazin-2-yl)-4-methylmorpholin-4-ium chloride
DPPA: Diphenylphosphoryl azide
EDCI: 1-ethyl-3-[3-(dimethylamino)propyl]carbodiimide
EEDQ: 1-Ethoxycarbonyl-2-ethoxy-1,2-dihydroquinoline
eq: equivalent
ESI: Electrospray ionization
EtOAc: Ethyl acetate
FA: Formic acid
h: hour(s)
HATU: 1-[Bis(dimethylamino)methylene]-1H-1,2,3-triazolo[4,5-b]pyridinium 3-oxid hexafluorophosphate
HFIP: 1,1,1,3,3,3-hexafluoropropan-2-ol
HOBT: 1-hydroxybenzotriazole
HPLC: High Performance Liquid Chromatography
IPA: 2-Propanol
LAH: Lithium aluminum hydride
LC-MS: Liquid Chromatography-Mass spectrometry
LiHMDS: Lithium bis(trimethylsilyl)amide
MeCN: Acetonitrile
MTBE: Methyl tert-butyl ether
NMM: 4-methylmorpholine
PE: Petroleum ether
Prep: Preparative
Py: Pyridine
RT: Retention time
rt: room temperature
TBAF: Tetra-n-butylammonium fluoride
TBS: tert-Butyldimethylsilyl
TEA: Triethylamine
tert-: tertiary
TFA: Trifluoroacetic acid
THF: Tetrahydrofuran
TLC: Thin layer chromatography
TMSOTf: Trimethylsilyl trifluoromethanesulfonate
TSTU: N,N,N,N′-Tetramethyl-O-(N-succinimidyl)uronium tetrafluoroborate

### 1. Synthesis of LP2

The synthesis of LP2 is shown below:

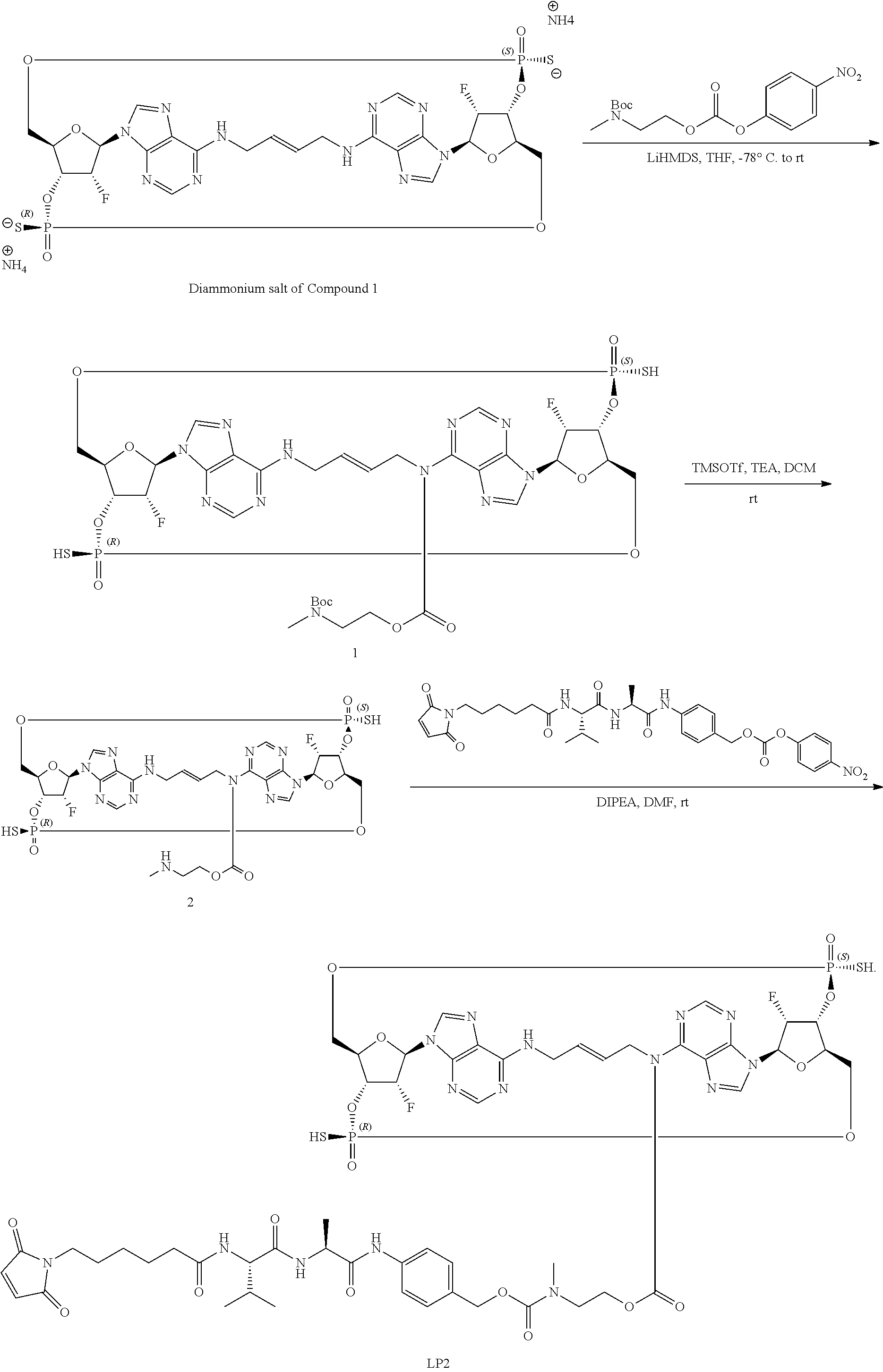

The synthesis of 2-[(tert-butoxycarbonyl)(methyl)amino]ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (1) is shown below:

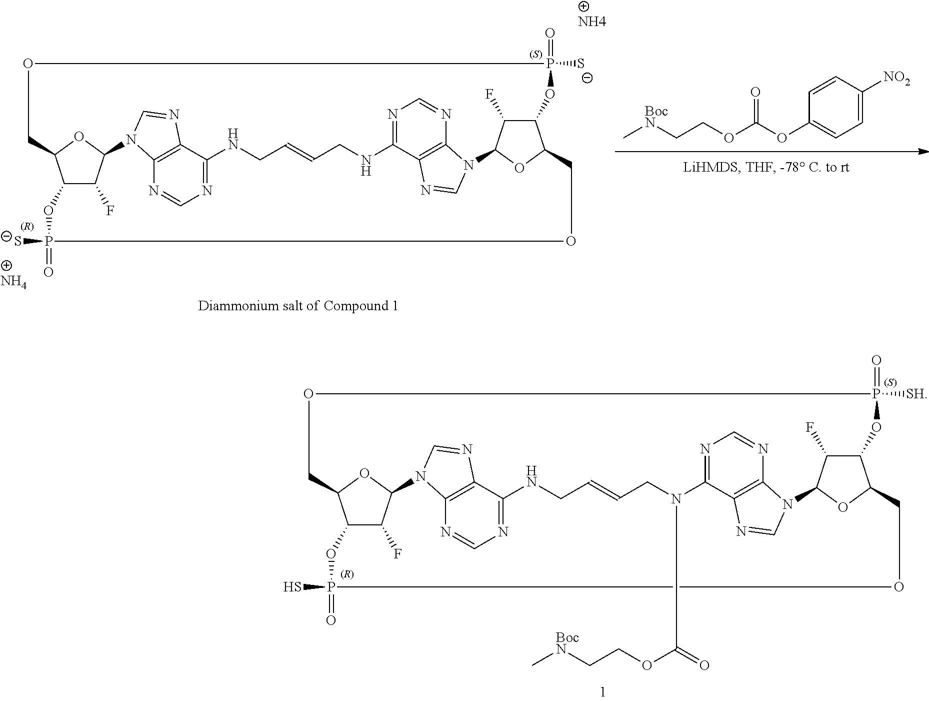

A solution of lithium bis(trimethylsilyl)amide (LiHMDS) (1.6 mmol) was added to a stirred solution of diammonium salt of Compound 1 (150 mg, 0.201 mmol) in tetrahydrofuran (THF) (10 mL) at −78° C. The resulting mixture was stirred at −78° C. for 30 min under nitrogen atmosphere. Then 1-[({2-[(tert-butoxycarbonyl)(methyl)amino]ethoxy}carbonyl)oxy]-4-nitrobenzene (68 mg, 0.201 mmol) was added to the resulting mixture at −78° C. The resulting mixture was slowly warmed to room temperature (rt) over 2 h. The reaction was quenched with AcOH (96.52 mg, 1.608 mmol). The resulting mixture was concentrated under reduced pressure. The residue was purified by Prep-TLC (CH₂Cl₂/MeOH 7:1) to obtain 100 mg of target product 1 as a white solid. LC-MS (ESI): 948.1 [M+H]⁺.

The synthesis of 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4. 1^{3,36}.1^{28,31}.0^{4,8}.0^{7,12}.0^{19,24}.0^{23,27}}]dotetraconta-5,7,9,11,15,19,21,23,25-nonaene-13-carboxylate (2) is shown below:

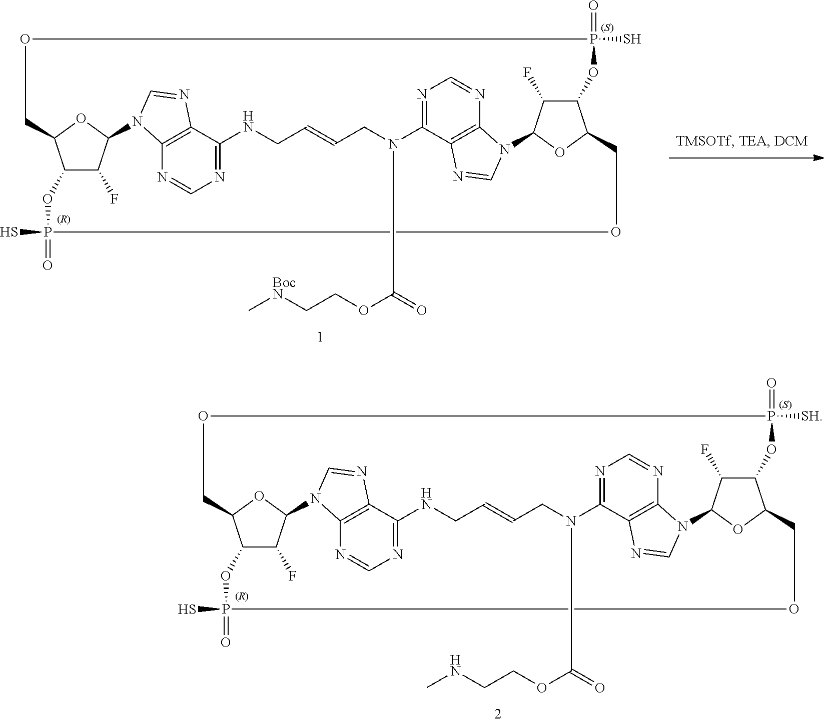

Triethylamine (TEA) (14.41 mg, 0.140 mmol) was added to a stirred solution of 2-[(tert-butoxycarbonyl)(methyl)amino]ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4. 1^{3,36}.1^{28,31}.0^{4,8}.0^{7,12}.0^{19,24}.0^{23,27}}]dotetraconta-5,7,9,11,15,19,21,23,25-nonaene-13-carboxylate (1) (27 mg, 0.028 mmol) in CH₂Cl₂ (2 mL). Then trimethylsilyl trifluoromethanesulfonate (TMSOTf) (44.32 mg, 0.196 mmol) was added to the resulting mixture at room temperature. The resulting mixture was stirred at room temperature for 30 min. The resulting mixture was concentrated under reduced pressure to provide target product 2 as a colorless oil, which was used for the next step without further purification. LC-MS (ESI): 848.1 [M+H]⁺.

The synthesis of 2-{[({4-[(2S)-2-[(2S)-2-[6-(2,5-dioxopyrrol-1-yl)hexanamido]-3-methylbutanamido]propanamido]phenyl}methoxy)carbonyl](methyl)amino}ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4. 1^{3,36}.1^{28,31}.0^{4,8}.0^{7,12}.0^{19,24}.0^{23,27}}]dotetraconta-5,7,9,11,15,19,21,23,25-nonaene-13-carboxylate (LP2) is shown in detail below:

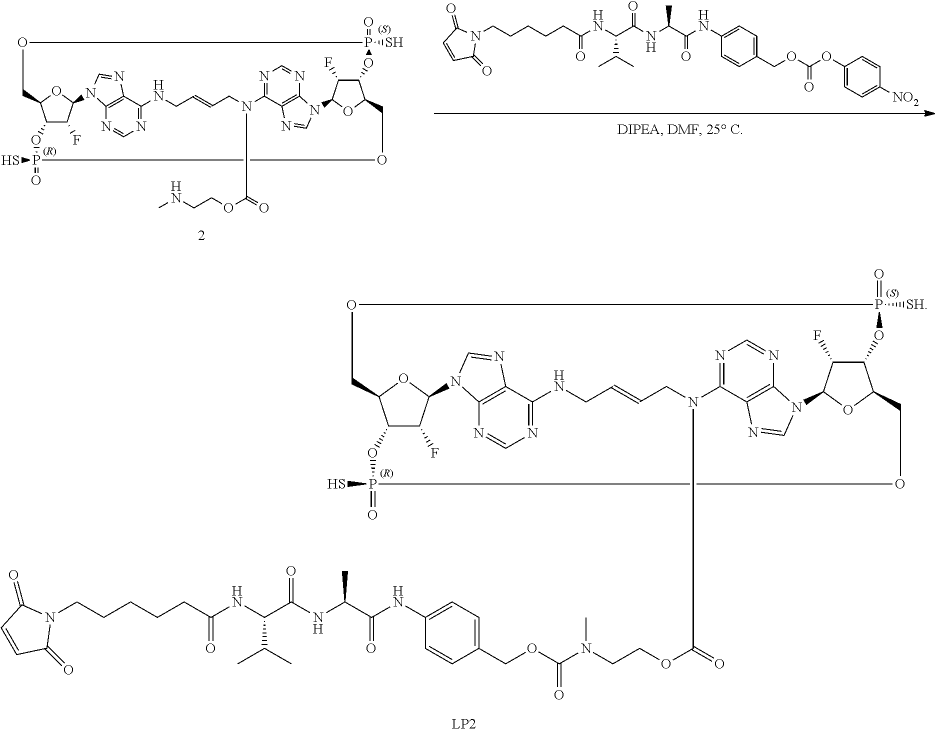

{4-[(2S)-2-[(2S)-2-[6-(2,5-dioxopyrrol-1-yl)hexanamido]-3-methylbutanamido]propanamido]phenyl}methyl 4-nitrophenyl carbonate (18.45 mg, 0.028 mmol) and N,N-Diisopropylethylamine (DIPEA) (7.32 mg, 0.056 mmol) were added to a stirred solution of 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4. 1^{3,36}.1^{28,31}.0^{4,8}.0^{7,12}.0^{19,24}.0^{23,27}}]dotetraconta-5,7,9,11,15,19,21,23,25-nonaene-13-carboxylate (2) (24 mg, 0.028 mmol) in N,N-dimethylformamide (DMF) (1 mL). The resulting mixture was stirred at room temperature for 14 h. The resulting mixture was concentrated under reduced pressure. The residue was purified by Prep-TLC (ethyl acetate (EtOAc) (1% TEA)/MeOH 3/1) and Prep-HPLC (Column: XBridge Prep Phenyl OBD Column, 19×250 mm, 5 μm; Mobile Phase A: Water (0.3% NH₄HCO₂), Mobile Phase B: acetonitrile (MeCN); Flow rate: 25 mL/min; Gradient: 25% B to 40% B in 10 min, 40% B; Wavelength: 254 nm; retention time (RT)=9 min) to afford target product LP2 (6.0 mg) as a white solid.

LC-MS (ESI): 1360.55 [M+H]⁺.

¹H NMR (300 MHz, Methanol-d₄) δ 8.97-8.85 (m, 1H), 8.84-8.62 (m, 2H), 8.12 (s, 1H), 7.69-7.51 (m, 2H), 7.40-7.14 (m, 2H), 6.88-6.72 (s, 2H), 6.54-6.20 (m, 2H), 6.11-5.97 (m, 1H), 5.95-5.18 (m, 3H), 5.09-4.95 (m, 2H), 4.91-4.86 (m, 2H), 4.72-4.57 (m, 3H), 4.57-4.46 (m, 3H), 4.48-4.36 (m, 2H), 4.36-4.24 (m, 1H), 4.24-4.12 (m, 2H), 4.11-3.90 (m, 2H), 3.72-3.54 (m, 1H), 3.55-3.41 (m, 4H), 2.85-2.68 (m, 3H), 2.41-2.23 (m, 2H), 2.18-2.02 (m, 1H), 1.79-1.50 (m, 5H) 1.50-1.41 (m, 3H), 1.36-1.22 (m, 2H), 1.09-0.88 (m, 7H).

³¹P NMR (162 MHz, Methanol-d₄) δ ppm 54.82, 55.19.

Analytical HPLC RT=3.60 min (Instrument: Shimadzu LC20AD; Column: HALO C18 (4.6 mm ID×100 mm); Mobile Phase A: Water (0.05% trifluoroacetic acid (TFA)), Mobile Phase B: MeCN (0.05% TFA); Flow rate: 1.5 mL/min; Temperature: 40° C.; Gradient: 10% B (t=0.01 min), 95% B (t=8 to 10 min); Wavelength: 254 nm).

### 2. Synthesis of LP1

The synthesis of LP1 is shown below:

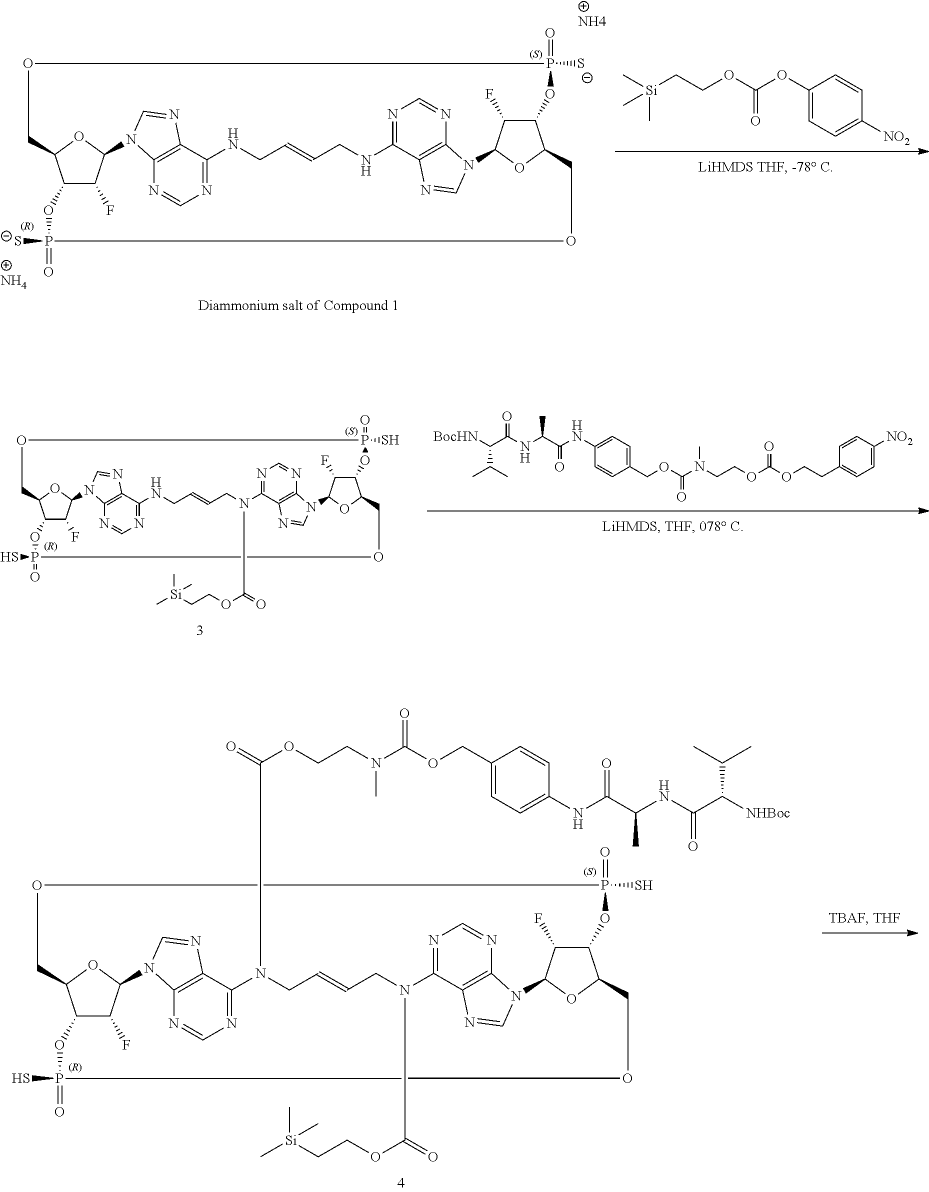

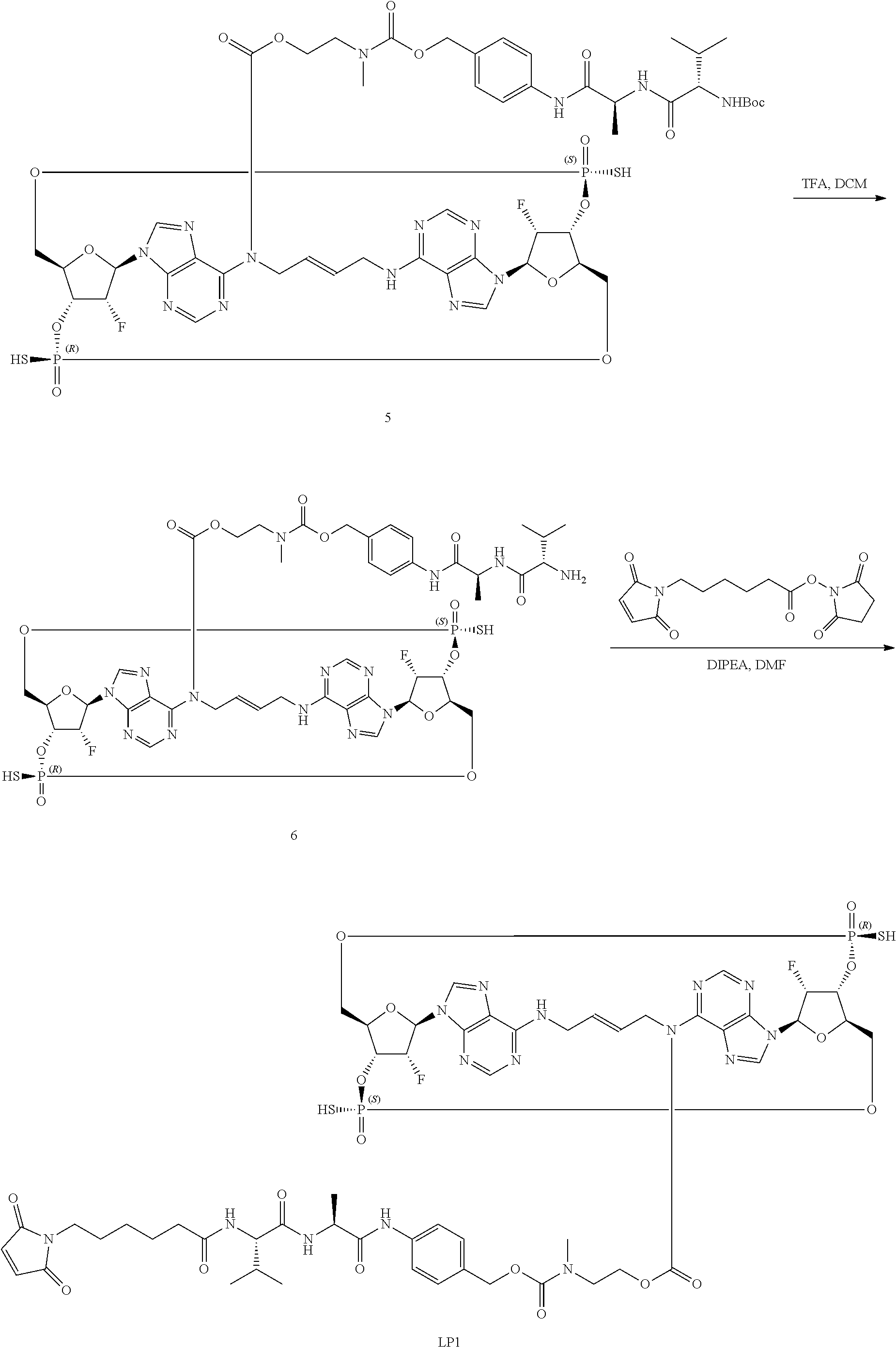

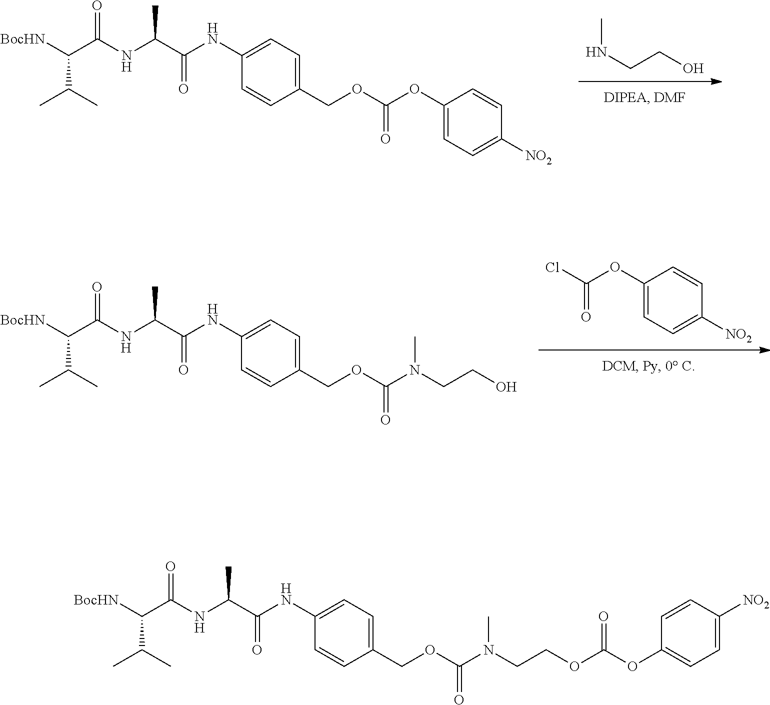

The synthesis of 4-nitrophenyl 2-(trimethylsilyl)ethyl carbonate is shown below:

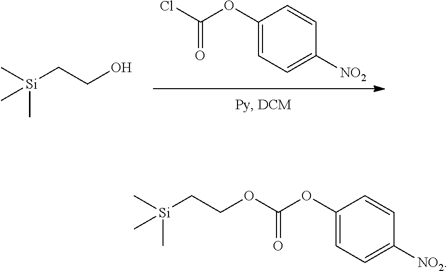

4-nitrophenyl carbonochloridate (3.8 g, 19 mmol) and pyridine (1.5 g, 19 mmol) were added to a solution of 2-(trimethylsilyl)ethanol (1.50 g, 12.7 mmol) in CH₂Cl₂ (20 mL). The resulting mixture was stirred at room temperature for 4 h. The resulting mixture was concentrated under reduced pressure. The residue was purified by silica gel column chromatography, eluted with petroleum ether/EtOAc (1:2). This resulted in 2.7 g of 4-nitrophenyl 2-(trimethylsilyl)ethyl carbonate as a light-yellow solid.

The synthesis of 2-(trimethylsilyl)ethyl (1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (3) is shown below:

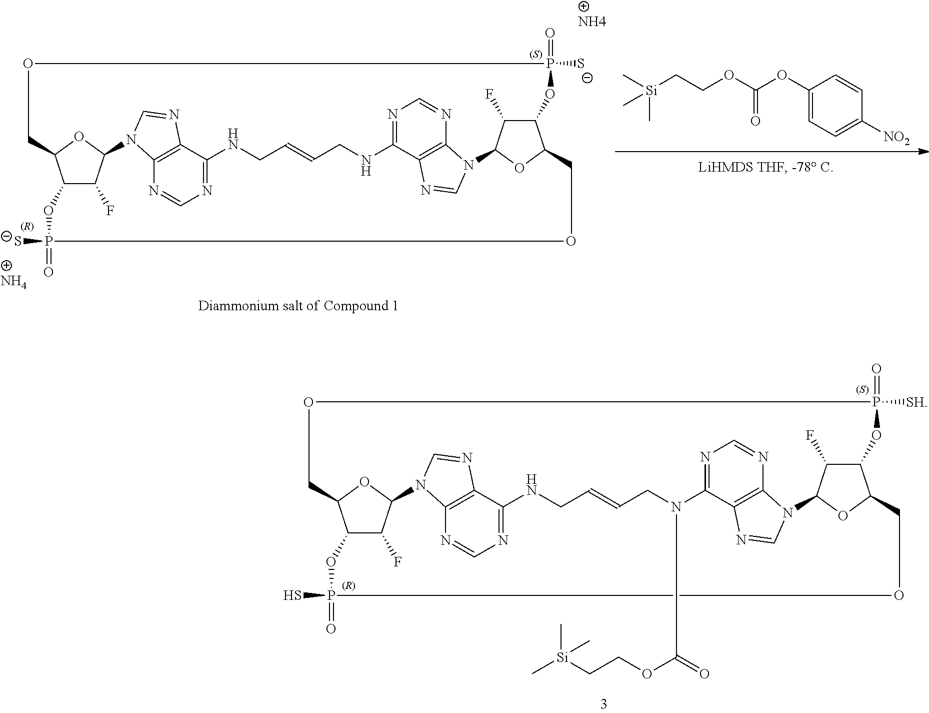

A solution of LiHMDS (0.80 mmol) was added to a solution of diammonium salt of Compound 1 (100 mg) in THF (3 mL) at −78° C. The resulting mixture was stirred at −78° C. for 30 min under nitrogen atmosphere. To the above mixture was added 4-nitrophenyl 2-(trimethylsilyl)ethyl carbonate (38 mg, 0.13 mmol) dropwise at −78° C. The resulting mixture was stirred at −78° C. for 1 h under nitrogen atmosphere. The reaction was quenched with AcOH (48 mg, 0.804 mmol). The resulting mixture was concentrated under reduced pressure. The residue was purified by Prep-TLC with CH₂Cl₂/MeOH (7:1). This resulted in 80 mg of target product 3 as a white solid. LC-MS (ESI): 891 [M+H]⁺.

The synthesis of tert-butyl N-[(1S)-1-{[(1S)-1-{[4-({[(2-hydroxyethyl)(methyl)carbamoyl]oxy}methyl)phenyl]carbamoyl}ethyl]carbamoyl}-2-methylpropyl]carbamate is shown below:

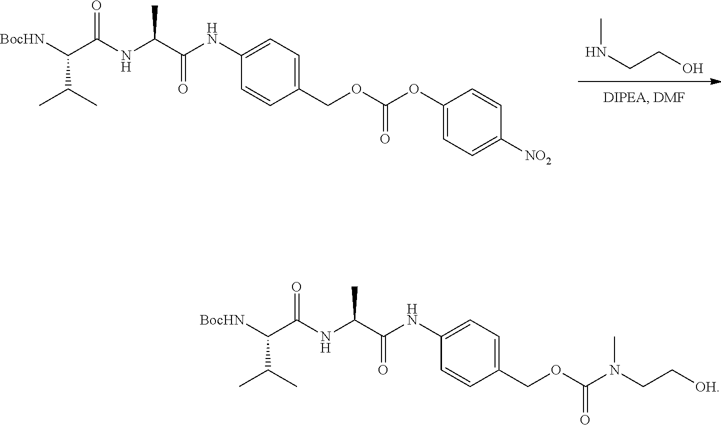

To a stirred solution of {4-[(2S)-2-[(2S)-2-[(tert-butoxycarbonyl)amino]-3-methylbutanamido]propanamido]phenyl}methyl 4-nitrophenyl carbonate (1.2 g, 2.1 mmol) and N-methyl-ethanolamine (161 mg, 2.15 mmol) in DMF (10 mL) were added DIPEA (555 mg, 4.30 mmol). The resulting mixture was stirred at room temperature for 2 h. The resulting mixture was concentrated under reduced pressure. The residue was purified by silica gel column chromatography, eluted with CH₂Cl₂/MeOH (12:1). This resulted in tert-butyl N-[(1S)-1-{[(1S)-1-{[4-({[(2-hydroxyethyl)(methyl)carbamoyl]oxy}methyl)phenyl]carbamoyl}ethyl]carbamoyl}-2-methylpropyl]carbamate (950 mg) as a white solid. LC-MS (ESI): 495.2 [M+H]⁺.

The synthesis of tert-butyl N-[(1S)-2-methyl-1-{[(1S)-1-{[4-({[methyl({2-[(4-nitrophenoxycarbonyl)oxy]ethyl})carbamoyl]oxy}methyl)phenyl]carbamoyl}ethyl]carbamoyl}propyl]c arbamate is shown below:

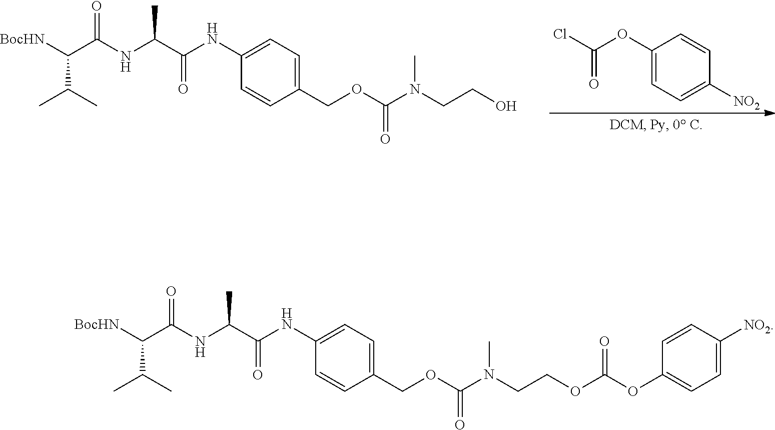 [SMILES: CC(C)[C@H](NC(=O)OC(C)(C)C)C(=O)N[C@@H](C)C(=O)Nc1ccc(COC(=O)N(C)CCO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N(C)CCOC(=O)Oc2ccc([N+](=O)[O-])cc2)cc1]

To a stirred solution of tert-butyl N-[(1S)-1-{[(1S)-1-{[4-({[(2-hydroxyethyl)(methyl)carbamoyl]oxy}methyl)phenyl]carbamoyl}ethyl]carbamoyl}-2-methylpropyl]carbamate (950 mg, 1.92 mmol) and 4-nitrophenyl carbonochloridate (581 mg, 2.88 mmol) in dichloromethane (DCM) (30 mL) were added pyridine (228 mg, 2.88 mmol). The resulting mixture was stirred at room temperature overnight under nitrogen atmosphere. The resulting mixture was concentrated under reduced pressure. The residue was purified by silica gel column chromatography, eluted with petroleum ether/EtOAc (1:2). This resulted in tert-butyl N-[(1S)-2-methyl-1-{[(1S)-1-{[4-({[methyl({2-[(4-nitrophenoxycarbonyl)oxy]ethyl})carbamoyl]oxy}methyl)phenyl]carbamoyl}ethyl]carbamoyl}propyl]c arbamate (1.1 g) as a white solid. LC-MS (ESI): 660.2 [M+H]⁺.

The synthesis of 13-(2-{[({4-[(2S)-2-[(2S)-2-[(tert-butoxycarbonyl)amino]-3-methylbutanamido]propanamido]phenyl}methoxy)carbonyl](methyl)amino}ethyl) 18-[2-(trimethylsilyl)ethyl](1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13,18-dicarboxylate (4) is shown below:

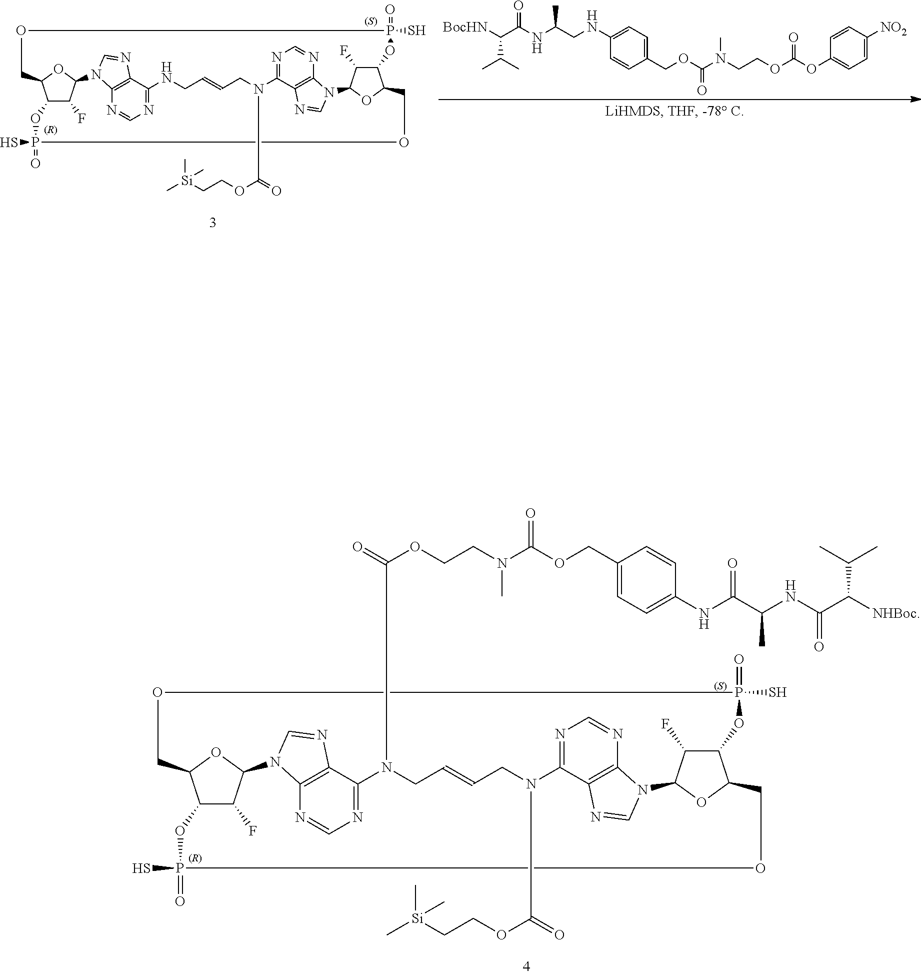

A solution of LiHMDS (0.54 mmol) was added to a mixture of 2-(trimethylsilyl)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]}dotetraconta-5,7,9,11,15,19,21,23,25-nonaene-13-carboxylate (3) (80 mg, 0.090 mmol) in THF (3 mL) at −78° C. The resulting mixture was stirred at −78° C. for 30 min under nitrogen atmosphere. To the above mixture was added tert-butyl N-[(1S)-2-methyl-1-{[(1S)-1-{[4-({[methyl({2-[(4-nitrophenoxycarbonyl)oxy]ethyl})carbamoyl]oxy}methyl)phenyl]carbamoyl}ethyl]carbamoyl}propyl]c arbamate (59.3 mg, 0.090 mmol) portionwise at −78° C. The resulting mixture was stirred at −78° C. for 2 h under nitrogen atmosphere. The reaction was quenched by the addition of AcOH at −78° C. The resulting mixture was concentrated under reduced pressure. The residue was purified by Prep-TLC (CH₂Cl₂/MeOH 7:1). This resulted in 60 mg of target product 4 as a white solid. LC-MS (ESI): 1411 [M+H]⁺.

### Synthesis of 2-{[({4-[(2S)-2-[(2S)-2-amino-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (5) is shown below

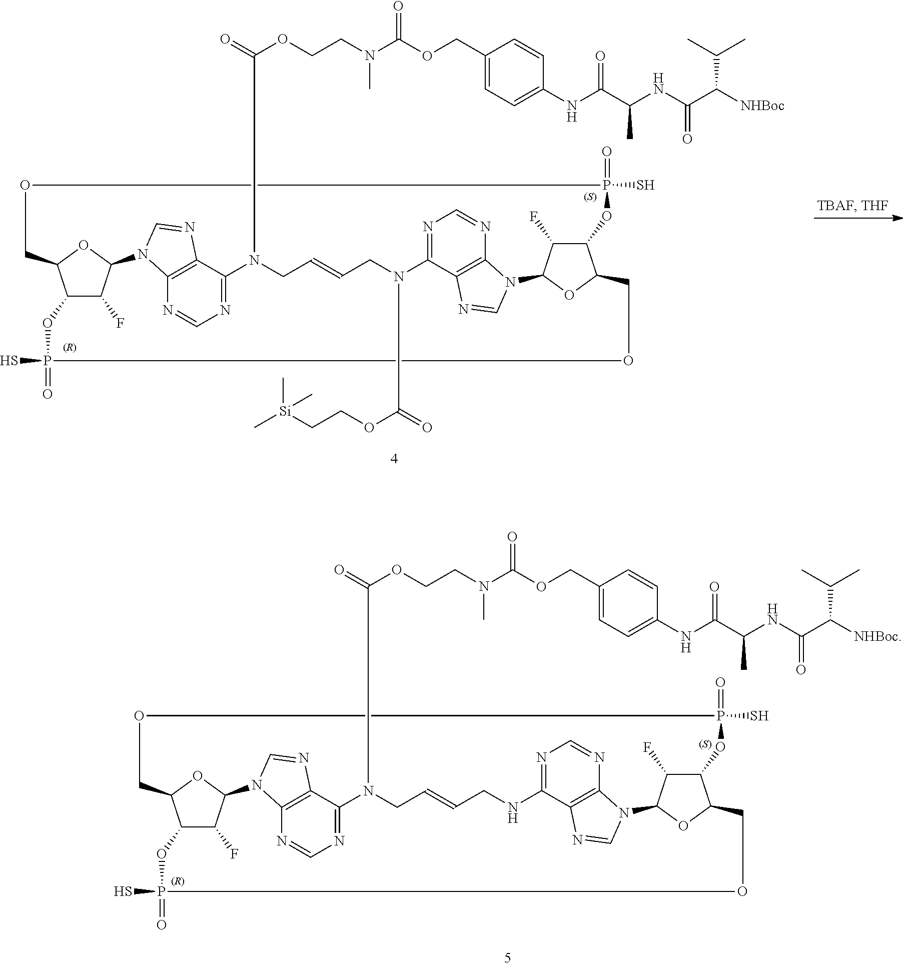

A solution of tetrabutylammonium fluoride (TBAF) (0.22 mmol) was added to a mixture of 13-(2-{[({4-[(2S)-2-[(2S)-2-[(tert-butoxycarbonyl)amino]-3-methylbutanamido]propanamido]phenyl}methoxy)carbonyl](methyl)amino}ethyl) 18-[2-(trimethylsilyl)ethyl](1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13,18-dicarboxylate (4) (60 mg, 0.043 mmol) in THF (3 mL). The resulting mixture was stirred at room temperature for 1 h. The resulting mixture was concentrated under reduced pressure. The residue was purified by Prep-TLC (EtOAc (1% TEA)/MeOH 3:1). This resulted in 48 mg of target product 5 as a white solid. LC-MS (ESI): 1267 [M+H]⁺.

### Synthesis of 2-{[({4-[(2S)-2-[(2S)-2-amino-3-methylbutanamido]propanamido]phenyl}methoxy)carbonyl](methyl)amino}ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (6) is shown below

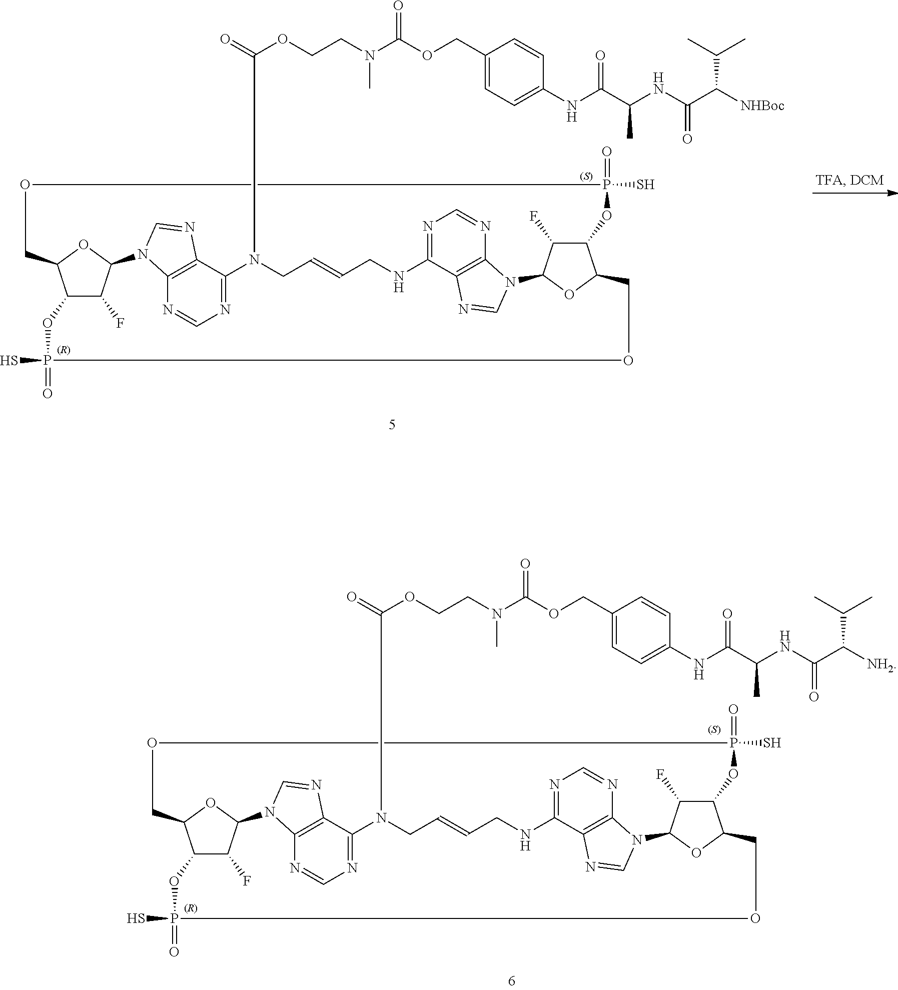

TFA (0.5 mL) was added to a mixture of 2-{[({4-[(2S)-2-[(2S)-2-[(tert-butoxycarbonyl)amino]-3-methylbutanamido]propanamido]phenyl}methoxy)carbonyl](methyl)amino}ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (5) (40 mg, 0.032 mmol) in DCM (3 mL). The resulting mixture was stirred at 0° C. for 1 h. The resulting mixture was concentrated under reduced pressure. This resulted in 60 mg (crude) of target product 6 as an oil. LC-MS (ESI): 1167 [M+H]⁺.

### Synthesis of (1-{[({4-[(2S)-2-[(2S)-2-amino-3-methylbutanamido]propanamido]phenyl}methoxy)carbonyl](methyl)amino}cyclopropyl)methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (LP1) is shown below

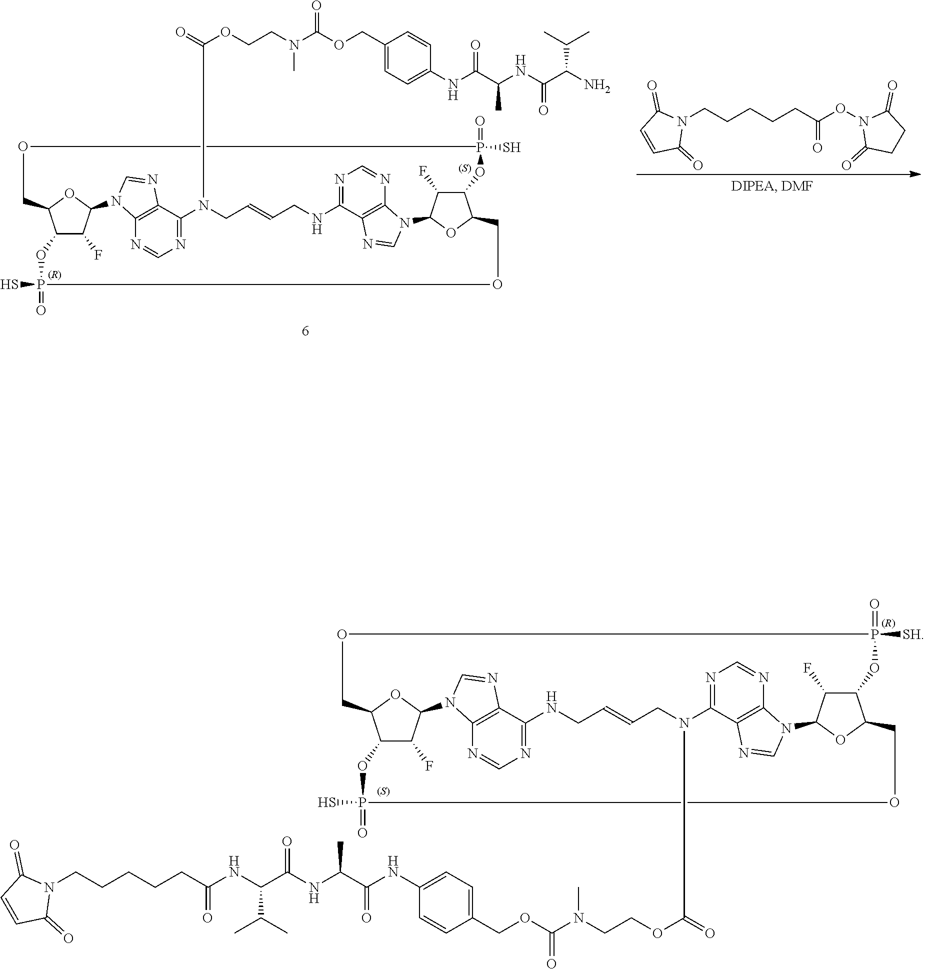

2,5-dioxopyrrolidin-1-yl 6-(2,5-dioxopyrrol-1-yl)hexanoate (21.1 mg, 0.068 mmol) and DIPEA (13.3 mg, 0.102 mmol) were added to a mixture of 2-{[({4-[(2S)-2-[(2S)-2-amino-3-methylbutanamido]propanamido]phenyl}methoxy)carbonyl](methyl)amino}ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaene-13-carboxylate (6) (40 mg, 0.034 mmol) in DMF (2 mL). The resulting mixture was stirred at room temperature for 1 h. The resulting mixture was concentrated under reduced pressure. The residue was purified by silica gel column chromatography, eluted with Prep-TLC (CH₂Cl₂/MeOH 5:1).

The crude product was purified by Prep-HPLC with the following conditions; Column: XBridge Prep Phenyl OBD Column, 19×250 mm, 5 μm; Mobile Phase A: Water (0.3% NH₄HCO₃), Mobile Phase B: MeCN; Flow rate: 25 mL/min; Gradient: 25% B to 40% B in 10 min, 40% B; Wavelength: 254 nm; RT=9 min. This resulted in 12.3 mg of target product LP1 as a white solid.

LC-MS (ESI): 1360.50 [M+H]⁺.

¹H NMR (400 MHz, Methanol-d₄) δ 9.21-9.09 (m, 1H), 8.80-8.70 (m, 1H), 8.59-8.45 (m, 1H), 8.20-7.73 (m, 3H), 7.64-7.54 (m, 2H), 7.35-7.27 (m, 1H), 7.27-7.18 (m, 1H), 6.83-6.77 (m, 2H), 6.55-6.41 (m, 1H), 6.40-6.16 (m, 1H), 5.88-5.63 (m, 2H), 5.60-5.49 (m, 1H), 5.48-5.29 (m, 1H), 5.15-4.91 (m, 5H), 4.75-4.68 (m, 3H), 4.67-4.59 (m, 2H), 4.58-4.47 (m, 3H), 4.46-4.38 (m, 2H), 4.33-4.14 (m, 3H), 4.11-4.02 (m, 1H), 4.01-3.93 (m, 1H), 3.71-3.60 (m, 2H), 3.57-3.43 (m, 3H), 2.80-2.69 (m, 3H), 2.34-2.26 (m, 2H), 2.15-2.04 (m, 1H), 1.70-1.54 (m, 4H), 1.50-1.44 (m, 3H), 1.36-1.27 (m, 3H), 1.03-0.95 (m, 6H).

³¹P NMR (162 MHz, Methanol-d₄) δ ppm 55.26, 55.71.

Analytical HPLC RT=7.70 min (Instrument: Shimadzu LC20AD; Column: XSelect HSS T3 (4.6 mm ID×100 mm); Mobile Phase A: Water (0.05% TFA), Mobile Phase B: MeCN (0.05% TFA); Flow rate: 1.2 mL/min; Temperature: 40° C.; Gradient: 10% B (t=0.01 min), 50% B (t=10 min), 95% B (t=12 to 14 min); Wavelength: 254 nm).

### 3. Synthesis of LP3

The synthesis of LP3 is shown below:

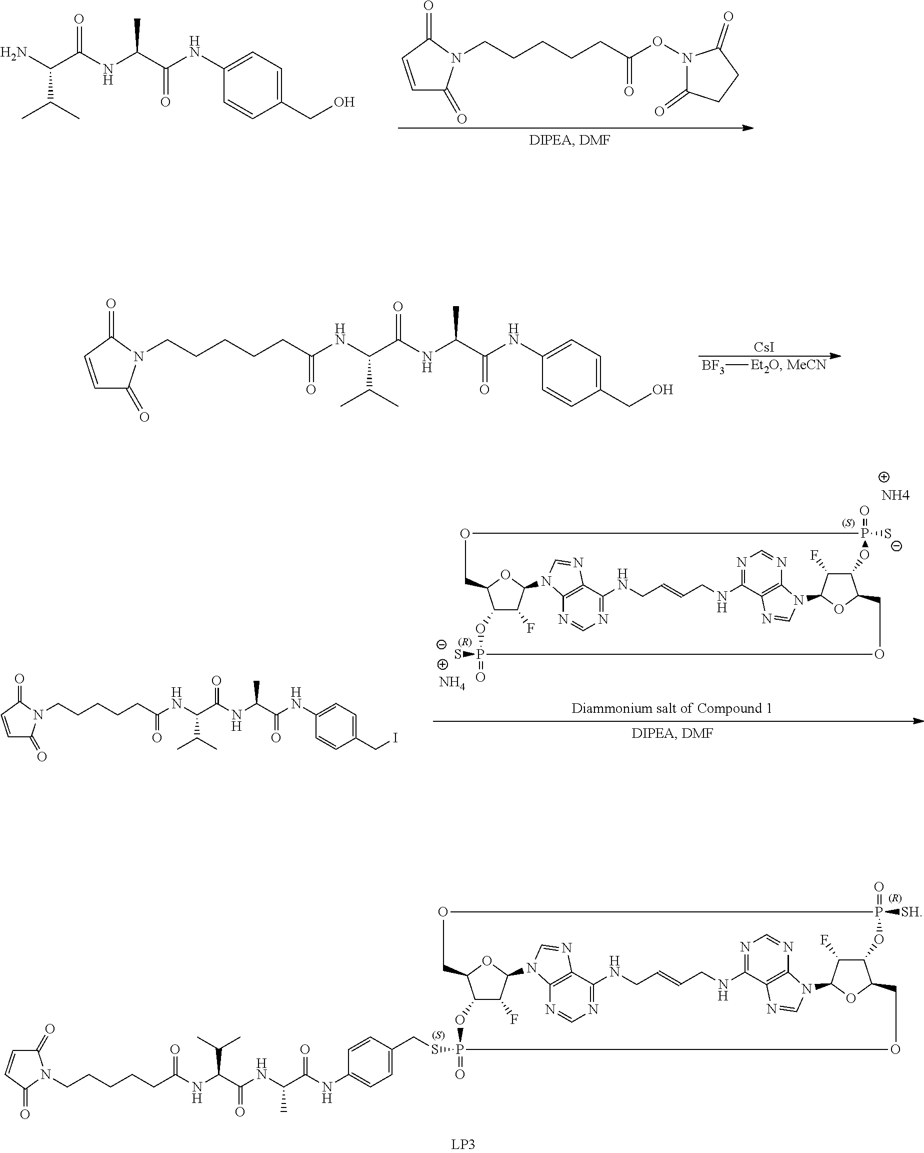

The synthesis of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide is shown below:

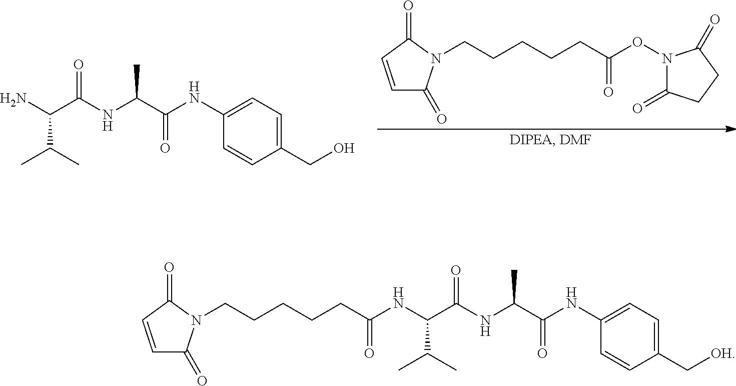 [SMILES: CC(C)[C@H](N)C(=O)N[C@@H](C)C(=O)Nc1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1]

DIPEA (1.32 g, 10.2 mmol) and 2,5-dioxopyrrolidin-1-yl 6-(2,5-dioxopyrrol-1-yl)hexanoate (1.58 g, 5.11 mmol) were added to a solution of (2S)-2-amino-N-[(1S)-1-{[4-(hydroxymethyl)phenyl]carbamoyl}ethyl]-3-methylbutanamide (1.5 g, 5.1 mmol) in DMF (40 mL). The resulting mixture was stirred at room temperature for 2 h. The resulting mixture was diluted with EtOAc, washed with water and concentrated. The residue was purified by silica gel column chromatography, eluted with CH₂Cl₂/MeOH (10:1). This resulted in 2.0 g of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide as an off-white solid. LC-MS (ESI): 487 [M+H]⁺.

The synthesis of 6-(2,5-dioxopyrrol-1-yl)-N-[(1S)-1-{[(1S)-1-{[4-(iodomethyl)phenyl]carbamoyl}ethyl]carbamoyl}-2-methylpropyl]hexanamide is shown below:

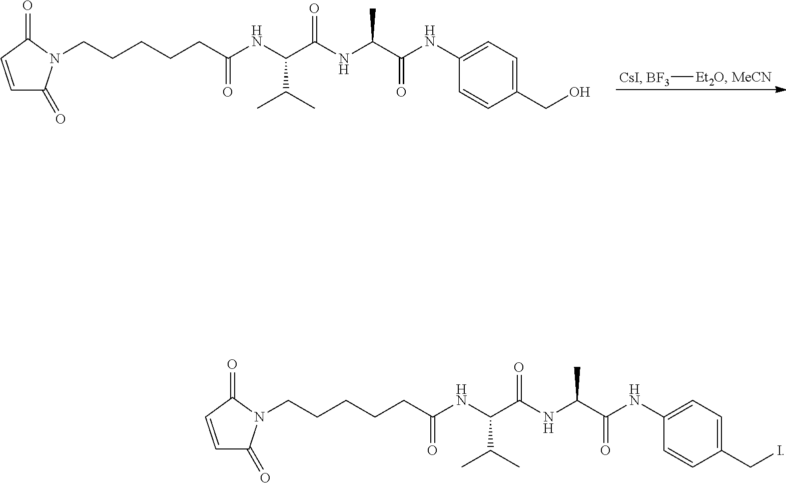

Cesium iodide (801 mg, 3.08 mmol) and boron trifluoride etherate (BF3 Et₂O) (438 mg, 3.08 mmol) were added to a solution of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (1.0 g, 2.06 mmol) in acetonitrile (10 mL). The resulting mixture was stirred at room temperature for 2 h. The resulting mixture was concentrated under reduced pressure. The residue was purified by silica gel column chromatography, eluted with CH₂Cl₂/ⁱPrOH (10:1). This resulted in 700 mg of 6-(2,5-dioxopyrrol-1-yl)-N-[(1S)-1-{[(1S)-1-{[4-(iodomethyl)phenyl]carbamoyl}ethyl]carbamoyl}-2-methylpropyl]hexanamide as a white solid. LC-MS (ESI): 597 [M+H]⁺.

The synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxopyrrol-1-yl)hexanamide (LP3) is shown below:

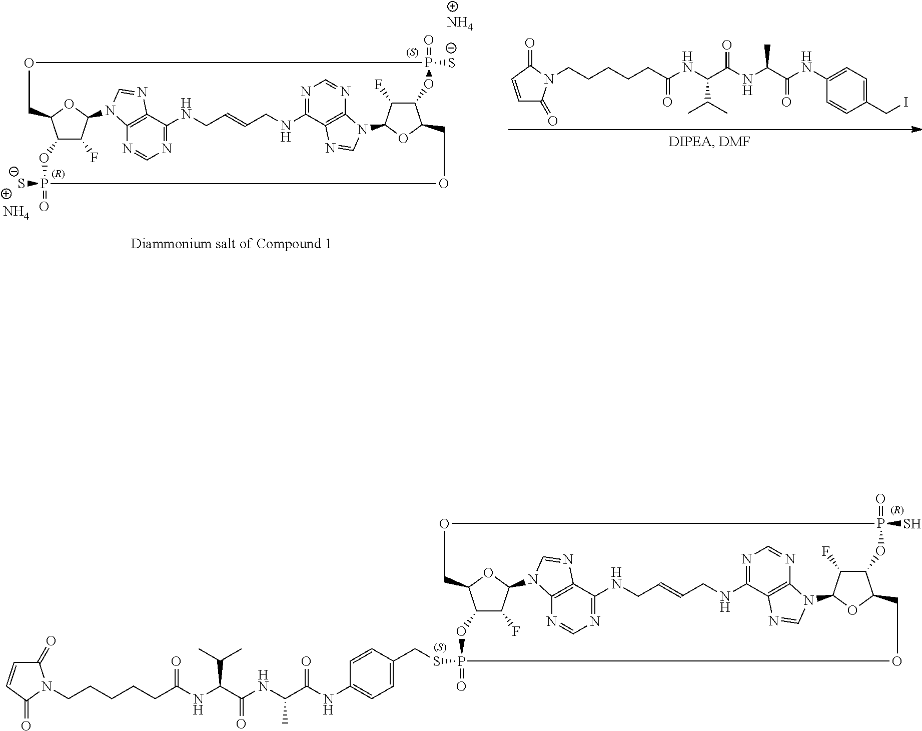

6-(2,5-dioxopyrrol-1-yl)-N-[(1S)-1-{[(1S)-1-{[4-(iodomethyl)phenyl]carbamoyl}ethyl]carbamoyl}-2-methylpropyl]hexanamide (480 mg, 0.805 mmol) and DIPEA (208 mg, 1.61 mmol) were added to a solution of diammonium salt of Compound 1 (600 mg, 0.805 mmol) in DMF (5 mL). The resulting mixture was stirred at room temperature for 2 h. The crude solution was purified by Prep-HPLC with the following conditions (Column: XBridge Prep OBD C18 Column, 19*×250 mm, 5 μm; Mobile Phase A: Water (0.05% FA), Mobile Phase B: MeCN; Flow rate: 25 mL/min; Gradient: 32% B to 32% B in 8 min, 32% B; Wavelength: 254 nm; RT=13 min). This resulted in 108 mg of target product LP3.

LC-MS (ESI): 1215.3 [M+H]⁺.

¹H NMR (400 MHz, Methanol-d₄) δ (ppm)=8.77-8.46 (m, 1H), 8.30-7.90 (m, 6H), 7.40 (br s, 2H), 7.22-6.85 (m, 2H), 6.77 (s, 2H), 6.45 (br d, J=14.5 Hz, 1H), 6.25 (br d, J=19.9 Hz, 1H), 5.90-5.10 (m, 6H), 4.75-3.50 (m, 14H), 3.46 (t, J=7.0 Hz, 2H), 2.28 (t, J=7.4 Hz, 2H), 2.16-2.04 (m, 1H), 1.70-1.50 (m, 4H), 1.47-1.37 (m, 3H), 1.35-1.24 (m, 2H), 0.97 (t, J=7.0 Hz, 6H).

Analytical HPLC RT=6.74 min (Instrument: Shimadzu LC20AD; Column: HALO C18 (4.6 mm ID×100 mm); Mobile Phase A: Water (0.05% TFA), Mobile Phase B: MeCN (0.05% TFA); Flow rate: 1.5 mL/min; Temperature: 40° C.; Gradient: 10% B (t=0.01 min), 70% B (t=10 min), 95% B (t=12 to 14 min); Wavelength: 254 nm).

### 4. Synthesis of LP4

The synthesis of LP4 is shown below:

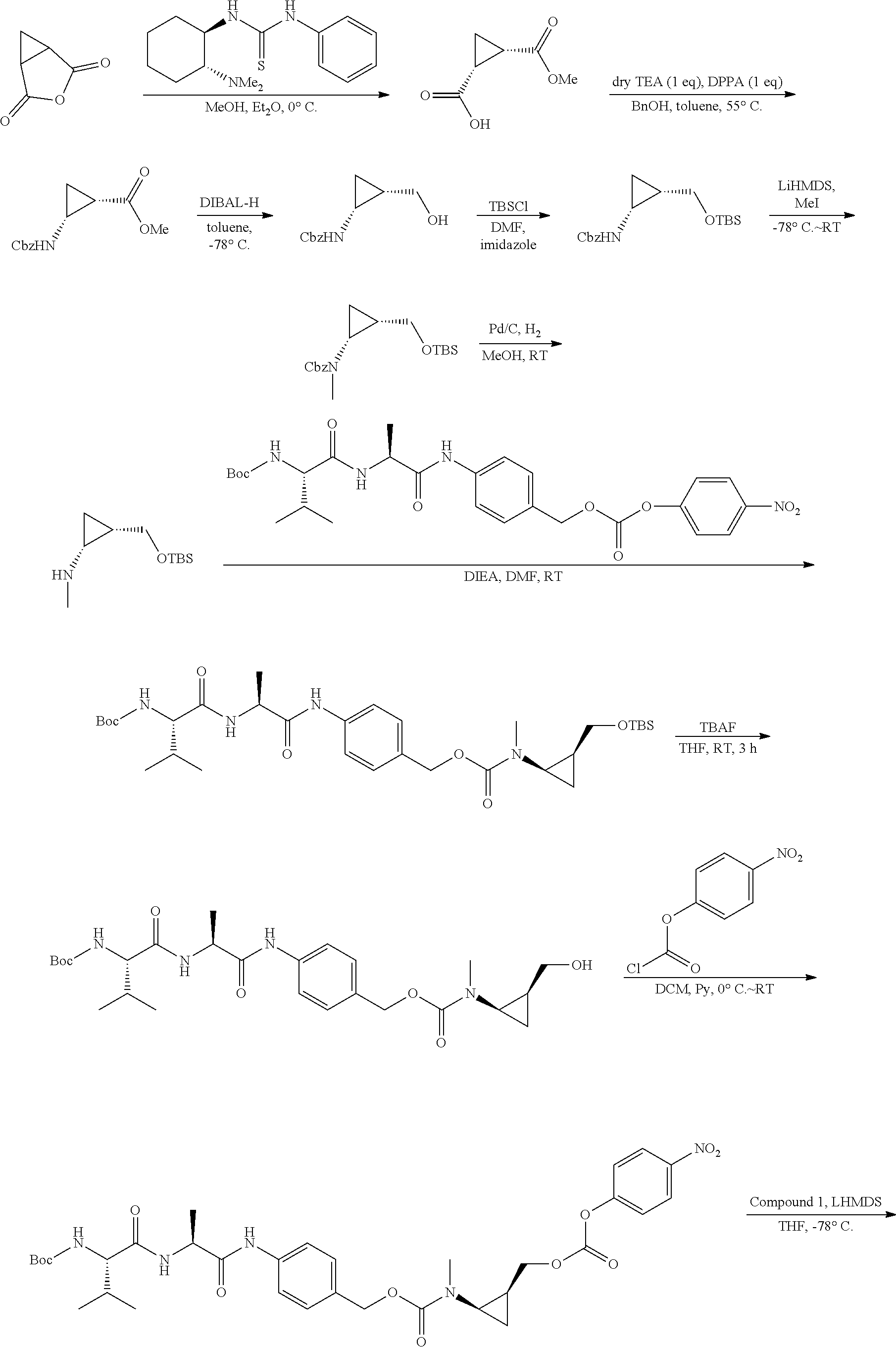

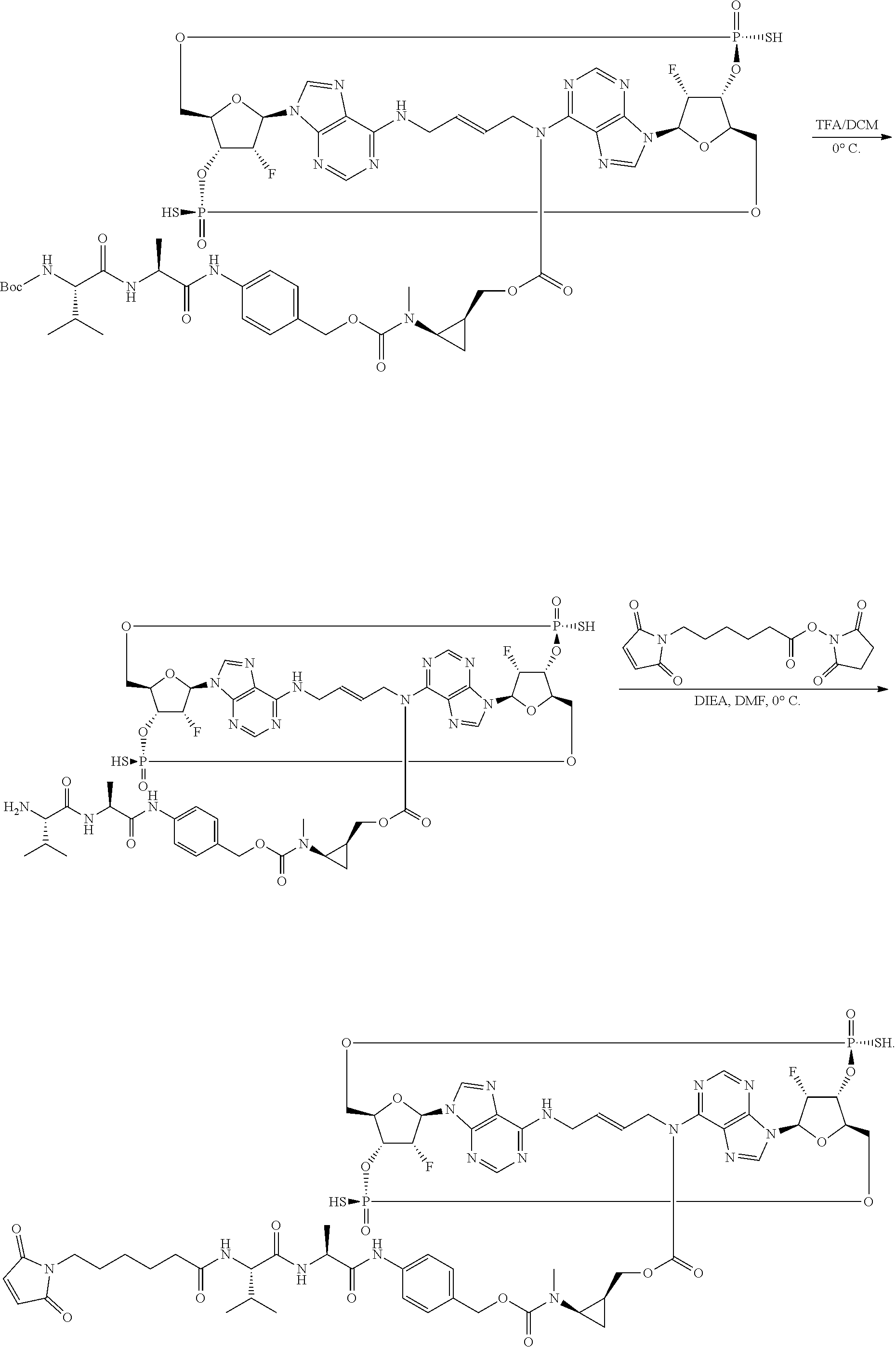

### Synthesis of (1R,2S)-2-(methoxycarbonyl)cyclopropane-1-carboxylic acid

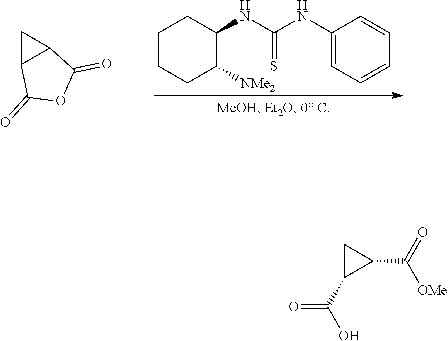 [SMILES: O=C1OC(=O)C2CC12 ; CN(C)[C@@H]1CCCC[C@H]1NC(=S)Nc1ccccc1 ; COC(=O)[C@H]1C[C@H]1C(=O)O]

3-[(1R,2R)-2-(dimethylamino)cyclohexyl]-1-phenylthiourea (618.80 mg, 2.231 mmol) was added to a solution of 3-oxabicyclo[3.1.0]hexane-2,4-dione (2.5 g, 22.3 mmol) in Et₂O (2.50 L) at room temperature. Then MeOH (9.03 mL) was added dropwise at 0° C. under nitrogen atmosphere. The resulting mixture was stirred at 25° C. for additional 14 h. The resulting mixture was concentrated under reduced pressure. The residue was dissolved in aqueous Na₂CO₃ solution (300 mL) and washed DCM (300 mL) to remove catalyst. The water layer was acidified to pH 1 with HCl. The mixture was extracted with EtOAc (300 ml×2). The organic layer was concentrated under reduced pressure. This resulted in (1R,2S)-2-(methoxycarbonyl)cyclopropane-1-carboxylic acid (2.5 g) as a yellow oil.

### Synthesis of methyl (1S,2R)-2-{[(benzyloxy)carbonyl]amino}cyclopropane-1-carboxylate

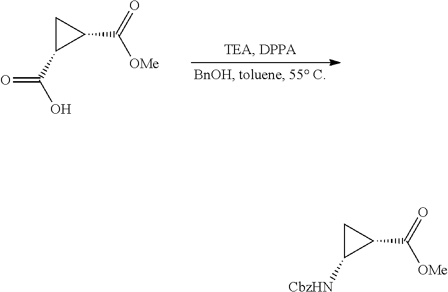 [SMILES: COC(=O)[C@H]1C[C@H]1C(=O)O ; COC(=O)[C@H]1C[C@H]1NC(=O)OCc1ccccc1]

To a solution of (1R,2S)-2-(methoxycarbonyl)cyclopropane-1-carboxylic acid (2500 mg, 17.3 mmol) in Toluene (150 mL) was added phenylmethanol (18757.51 mg, 173.5 mmol), TEA (1755.29 mg, 17.3 mmol) and DPPA(4.7 g, 17.3 mmol) at room temperature. The resulting mixture was stirred at 55° C. for 14 h. The resulting mixture was concentrated under reduced pressure. The crude product was purified by reverse phase flash with the following conditions (Column: C18; mobile phase A: Water (1% FA), mobile phase B: MeCN; gradient: 5% B to 25% B in 30 min; 220/254 nm) to afford methyl (1S,2R)-2-{[(benzyloxy)carbonyl]amino}cyclopropane-1-carboxylate (1000 mg) as a white solid. LC-MS (ESI): 250.1 [M+H]⁺.

### Synthesis of benzyl N-[(1R,2S)-2-(hydroxymethyl)cyclopropyl]carbamate

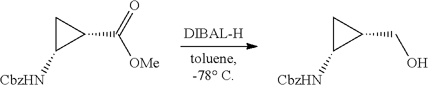 [SMILES: COC(=O)[C@H]1C[C@H]1NC(=O)OCc1ccccc1 ; O=C(N[C@@H]1C[C@@H]1CO)OCc1ccccc1]

To a solution of methyl (1S,2R)-2-{[(benzyloxy)carbonyl]amino}cyclopropane-1-carboxylate (1000 mg, 4.012 mmol) in Toluene (6 mL) was added dropwise DIBAL-H (16.048 mmol) at −78° C. The resulting mixture was stirred at −78° C. for 3 h under N₂ atmosphere. The reaction was quenched by AcOH. The mixture was diluted with EtOAc and washed with H₂O. The resulting mixture was concentrated under reduced pressure. The crude product was purified by reverse phase flash with the following conditions (Column: C18; mobile phase A: Water (1% FA), mobile phase B: MeCN; gradient: 5 B to 20 B in 30 min; 220/254 nm.) to afford benzyl N-[(1R,2S)-2-(hydroxymethyl)cyclopropyl]carbamate (400 mg) as a colorless oil. LC-MS (ESI): 222.1 [M+H]⁺.

### Synthesis of benzyl N-[(1R,2S)-2-{[(tert-butyldimethylsilyl)oxy]methyl}cyclopropyl]carbamate

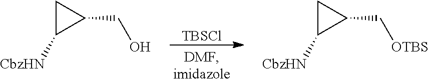 [SMILES: O=C(N[C@@H]1C[C@@H]1CO)OCc1ccccc1 ; "TBSCl" ; CC(C)(C)[Si](C)(C)OC[C@H]1C[C@H]1NC(=O)OCc1ccccc1]

To a solution of benzyl N-[(1R,2S)-2-(hydroxymethyl)cyclopropyl]carbamate (800 mg, 3.616 mmol), and Imidazole (369.23 mg, 5.424 mmol) in DMF (5 mL) was added t-butyldimethylchlorosilane (817.44 mg, 5.424 mmol). The resulting mixture was stirred at 25° C. for 14 h. The resulting mixture was concentrated under vacuum. The residue was purified by silica gel column chromatography, eluted with PE:EtOAc (5:1) to afford benzyl N-[(1R,2S)-2-{[(tert-butyldimethylsilyl)oxy]methyl}cyclopropyl]carbamate (600 mg) as a colorless oil. LC-MS (ESI): 336.1 [M+H]⁺.

### Synthesis of benzyl N-[(1R,2S)-2-{[(tert-butyldimethylsilyl)oxy]methyl}cyclopropyl]-N-methylcarbamate

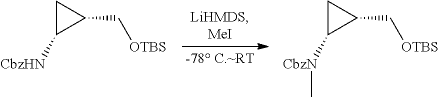 [SMILES: CC(C)(C)[Si](C)(C)OC[C@H]1C[C@H]1NC(=O)OCc1ccccc1 ; CN(C(=O)OCc1ccccc1)[C@@H]1C[C@@H]1CO[Si](C)(C)C(C)(C)C]

To a solution of benzyl N-[(1R,2S)-2-{[(tert-butyldimethylsilyl)oxy]methyl}cyclopropyl]carbamate (300 mg, 0.894 mmol) in THF (20 mL) was added dropwise LiHMDS (2.682 mmol) at −78° C. under nitrogen. The mixture was stirred at −78° C. for 30 min under nitrogen atmosphere. To the above mixture was added iodomethane (761.48 mg, 5.364 mmol) at −78° C. The resulting mixture was slowly warmed to room temperature over two hours. The reaction was quenched by AcOH. The resulting mixture was concentrated under reduced pressure. The residue was purified by Prep-TLC (PE:EtOAc=5:1) to afford benzyl N-[(1R,2S)-2-{[(tert-butyldimethylsilyl)oxy]methyl}cyclopropyl]-N-methylcarbamate (250 mg) as a colorless oil. LC-MS (ESI): 350.2 [M+H]⁺.

### Synthesis of (1R,2S)-2-{[(tert-butyldimethylsilyl)oxy]methyl}-N-methylcyclopropan-1-amine

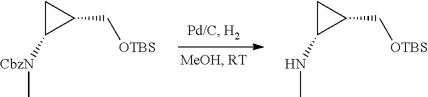 [SMILES: CN(C(=O)OCc1ccccc1)[C@@H]1C[C@@H]1CO[Si](C)(C)C(C)(C)C ; CN[C@@H]1C[C@@H]1CO[Si](C)(C)C(C)(C)C]

To a solution of benzyl N-[(1R,2S)-2-{[(tert-butyldimethylsilyl)oxy]methyl}cyclopropyl]-N-methylcarbamate (400 mg, 1.144 mmol) in MeOH (5 mL) was added Pd/C (80 mg, 10%). The resulting mixture was stirred at 25° C. for 14 h under H₂ atmosphere. The resulting mixture was filtered, the filter cake was washed with MeOH. The filtrate was concentrated under reduced pressure. This resulted in (1R,2S)-2-{[(tert-butyldimethylsilyl)oxy]methyl}-N-methylcyclopropan-1-amine (200 mg) as a colorless oil. LC-MS (ESI): 216.2 [M+H]⁺.

### Synthesis of {4-[(2S)-2-[(2S)-2-{[(tert-butoxy)carbonyl]amino}-3-methylbutanamido]propanamido]phenyl}methyl N-[(1R,2S)-2-{[(tert-butyldimethylsilyl)oxy]methyl}cyclopropyl]-N-methylcarbamate

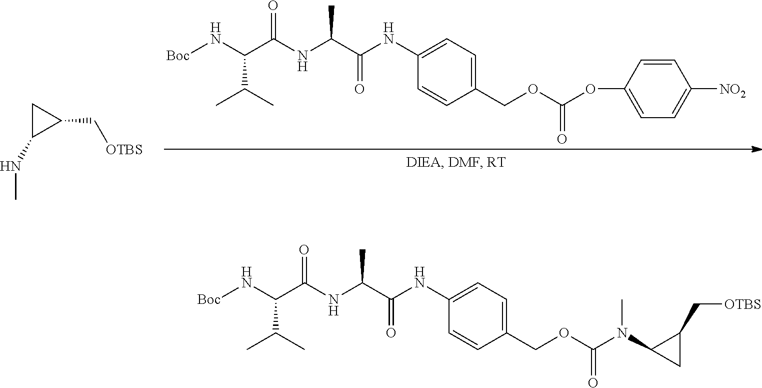 [SMILES: CN[C@@H]1C[C@@H]1CO[Si](C)(C)C(C)(C)C ; CC(C)[C@H](NC(=O)OC(C)(C)C)C(=O)N[C@@H](C)C(=O)Nc1ccc(COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N(C)[C@@H]2C[C@@H]2CO[Si](C)(C)C(C)(C)C)cc1]

To a solution of (1R,2S)-2-{[(tert-butyldimethylsilyl)oxy]methyl}-N-methylcyclopropan-1-amine (231.38 mg, 1.074 mmol) and tert-butyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (400 mg, 0.716 mmol) in DMF (4 mL) was added DIEA (370.21 mg, 2.864 mmol). The resulting mixture was stirred at 25° C. for 14 h. The filtrate was concentrated under reduced pressure. The residue was purified by Prep-TLC (DCM:EtOAc=4:1) to afford {4-[(2S)-2-[(2S)-2-{[(tert-butoxy)carbonyl]amino}-3-methylbutanamido]propanamido]phenyl}methyl N-[(1R,2S)-2-{[(tert-butyldimethylsilyl)oxy]methyl}cyclopropyl]-N-methylcarbamate (350 mg) as a yellow solid. LC-MS (ESI): 635.4 [M+H]⁺.

### Synthesis of {4-[(2S)-2-[(2S)-2-{[(tert-butoxy)carbonyl]amino}-3-methylbutanamido]propanamido]phenyl}methyl N-[(1R,2S)-2-(hydroxymethyl)cyclopropyl]-N-methylcarbamate

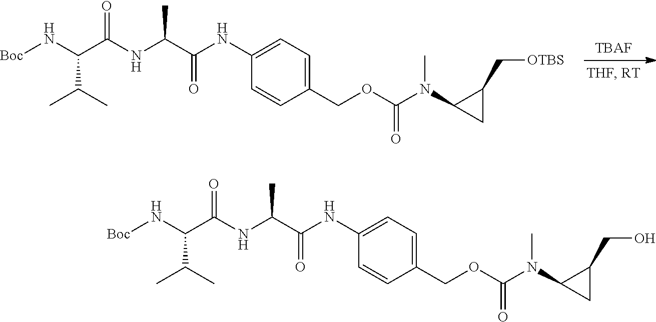 [SMILES: CC(C)[C@H](NC(=O)OC(C)(C)C)C(=O)N[C@@H](C)C(=O)Nc1ccc(COC(=O)N(C)[C@@H]2C[C@@H]2CO[Si](C)(C)C(C)(C)C)cc1 ; "TBAF" ; CC(C)[C@H](NC(=O)OC(C)(C)C)C(=O)N[C@@H](C)C(=O)Nc1ccc(COC(=O)N(C)[C@@H]2C[C@@H]2CO)cc1]

To a solution of {4-[(2S)-2-[(2S)-2-{[(tert-butoxy)carbonyl]amino}-3-methylbutanamido]propanamido]phenyl}methyl N-[(1R,2S)-2-{[(tert-butyldimethylsilyl)oxy]methyl}cyclopropyl]-N-methylcarbamate (300 mg, 0.473 mmol) in THF (3 mL) was added TBAF (370.64 mg, 1.419 mmol) at room temperature. The resulting mixture was stirred at room temperature for 3 h. The resulting mixture was concentrated under reduced pressure. The residue was purified by Prep-TLC (PE/EtOAc=1:4) to afford {4-[(2S)-2-[(2S)-2-[(tert-butoxycarbonyl)amino]-3-methylbutanamido]propanamido]phenyl}methyl N-[(1R,2S)-2-(hydroxymethyl)cyclopropyl]-N-methylcarbamate (220 mg) as a white solid. Column: CHIRAL ART Cellulose-SC, 2*25 cm, 5 μm; Mobile Phase A: Hex(0.5% 2M NH₃-MeOH)-HPLC, Mobile Phase B: EtOH:DCM=1: 1-HPLC; Flow rate: 20 mL/min; Gradient: 30% B to 30% B in 16 min; Wavelength: 220/254 nm; LC-MS (ESI): 521.3 [M+H]⁺.

### Synthesis of [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl 4-nitrophenyl carbonate

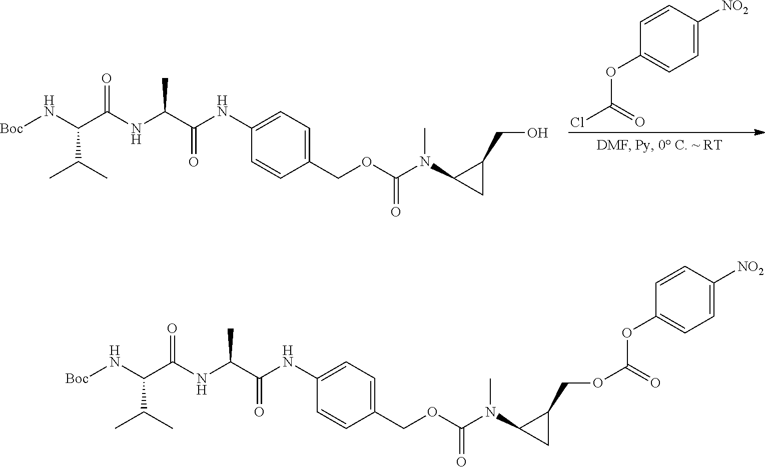 [SMILES: CC(C)[C@H](NC(=O)OC(C)(C)C)C(=O)N[C@@H](C)C(=O)Nc1ccc(COC(=O)N(C)[C@@H]2C[C@@H]2CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N(C)[C@@H]2C[C@@H]2COC(=O)Oc2ccc([N+](=O)[O-])cc2)cc1]

To a solution of 4-nitrophenyl carbonochloridate (309.72 mg, 1.536 mmol) and {4-[(2S)-2-[(2S)-2-[(tert-butoxycarbonyl)amino]-3-methylbutanamido]propanamido]phenyl}methyl N-[(1R,2S)-2-(hydroxymethyl)cyclopropyl]-N-methylcarbamate (400 mg, 0.768 mmol) in DCM (3 mL) was added Pyridine (121.55 mg, 1.536 mmol). The mixture was stirred at 25° C. for 6 h under N₂ atmosphere. The resulting mixture was concentrated under reduced pressure. The residue was purified by silica gel column chromatography, eluted with (PE:EtOAc=1:2) to afford [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl 4-nitrophenyl carbonate (240 mg) as a white solid. LC-MS (ESI): 686.3 [M+H]⁺.

### Synthesis of [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

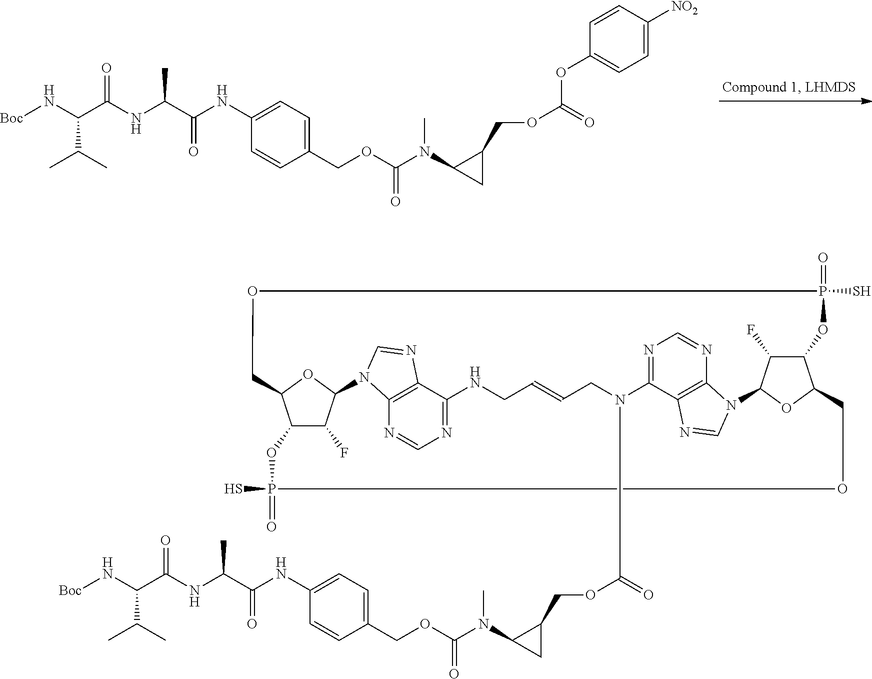 [SMILES: CC(C)[C@H](NC(=O)OC(C)(C)C)C(=O)N[C@@H](C)C(=O)Nc1ccc(COC(=O)N(C)[C@@H]2C[C@@H]2COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

The solution of Compound 1 (50 mg, 0.067 mmol) in THF (3 mL) was treated with LiHMDS (0.402 mmol) at −78° C. for 30 min under nitrogen atmosphere followed by the addition of solution of [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl 4-nitrophenyl carbonate (46 mg, 0.067 mmol) in THF(0.5 mL) dropwise at −78° C. The mixture was slowly warmed to room temperature over an hour. The reaction was quenched by AcOH. The resulting mixture was concentrated under reduced pressure. The residue was purified by Prep-TLC (EtOAc(1% TEA): MeOH=4:1) to afford [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg) as a white solid. LC-MS (ESI): 1293.4 [M+H]⁺.

### Synthesis of [(1S,2R)-2-{[({4-[(2S)-2-[(2S)-2-amino-3-methylbutanamido]propanamido]phenyl}methoxy)carbonyl](methyl)amino}cyclopropyl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

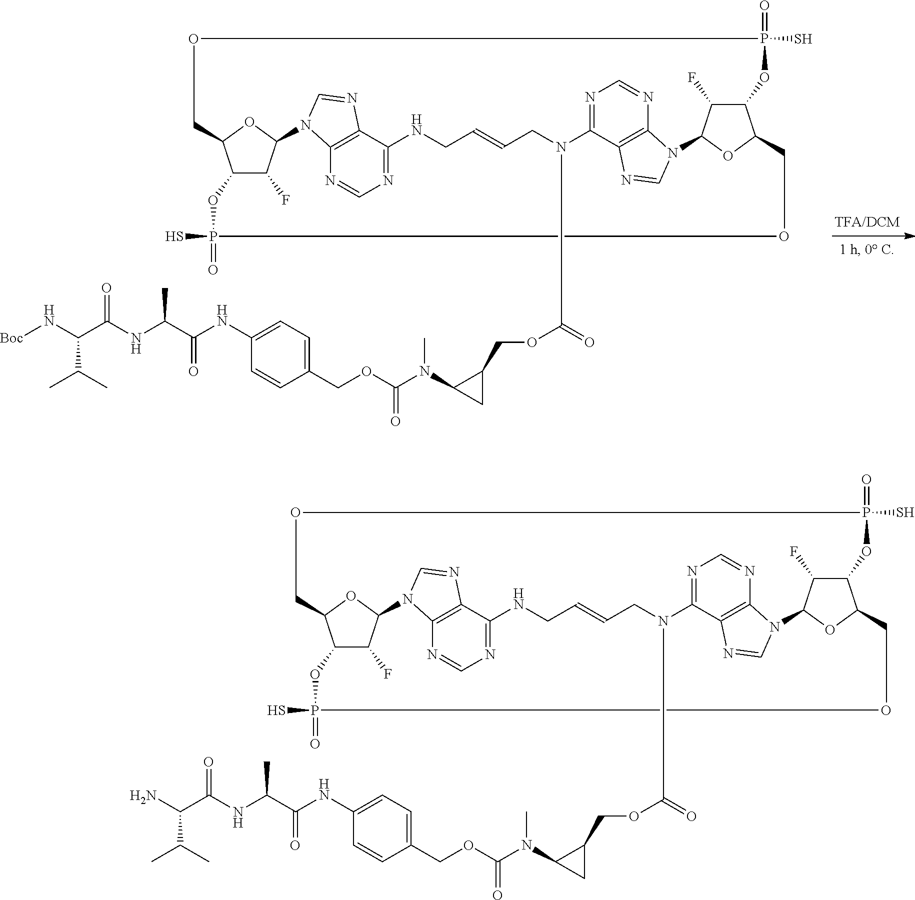 [SMILES: CC(C)[C@H](NC(=O)OC(C)(C)C)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

The solution of [(1S,2R)-2-{[({4-[(2S)-2-[(2S)-2-{[(tert-butoxy)carbonyl]amino}-3-methylbutanamido]propanamido]phenyl}methoxy)carbonyl](methyl)amino}cyclopropyl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (35 mg, 0.027 mmol) and TFA (0.3 mL) in DCM (1.8 mL) was stirred at 0° C. for 1 h. The resulting mixture was concentrated under vacuum. The crude product was used in the next step directly without further purification. LC-MS (ESI): 1193.3 [M+H]⁺.

### Synthesis of [(1S,2R)-2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}cyclopropyl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP4)

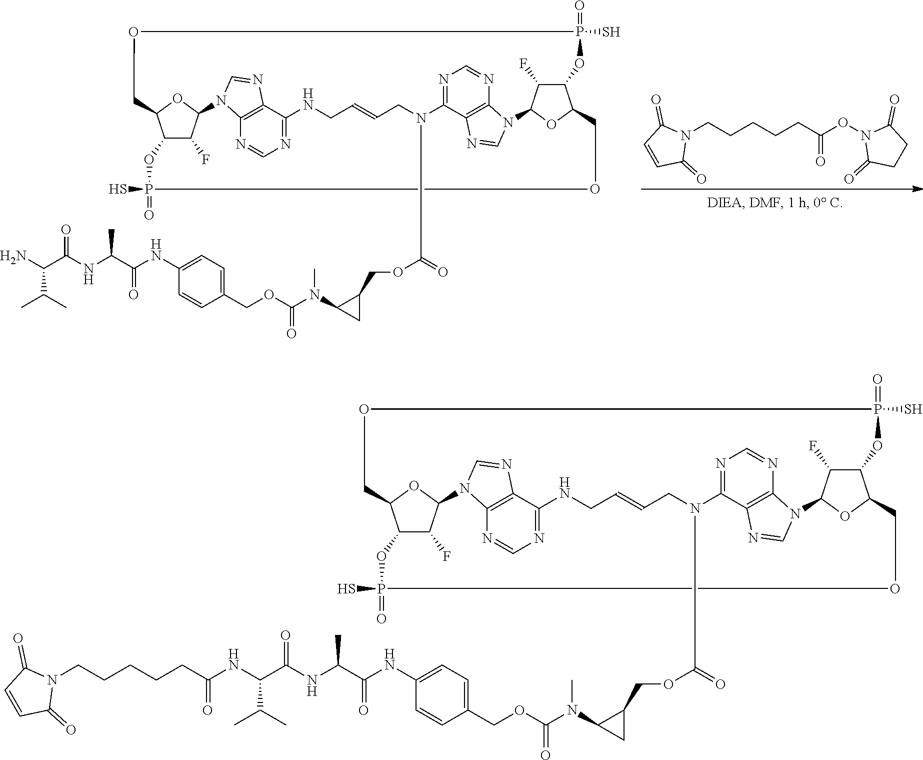 [SMILES: CC(C)[C@H](N)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)[C@@H]2C[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of [(1S,2R)-2-{[({4-[(2S)-2-[(2S)-2-amino-3-methylbutanamido]propanamido]phenyl}methoxy)carbonyl](methyl)amino}cyclopropyl]methyl (1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (35 mg, 0.029 mmol) and 2,5-dioxopyrrolidin-1-yl 6-(2,5-dioxopyrrol-1-yl)hexanoate (9.04 mg, 0.029 mmol) in DMF (3 mL) was added DIEA (18.96 mg, 0.145 mmol). The mixture was stirred at 25° C. for 2 h. The resulting mixture was concentrated under vacuum. The residue was purified by Prep-TLC (EtOAc(1% TEA):MeOH=4:1) and Prep-HPLC with the following conditions (Column: Xbridge Prep Phenyl OBD Column, 19×250 mm, 5 μm; Mobile Phase A: Water (50 mmol HCO₂NH₄), Mobile Phase B: MeCN; Flow rate: 25 mL/min; Gradient: 25% B to 35% B in 10 min, 35% B; Wavelength: 254 nm) to afford [(1S,2R)-2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}cyclopropyl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP4) (7.6 mg) as a white solid.

LC-MS (ESI): 1386.40 [M+H]⁺.

¹H NMR (300 MHz, Methanol-d₄) δ 8.99-8.61 (m, 3H), 8.20-8.03 (m, 1H), 7.65-7.49 (m, 2H), 7.35-7.14 (m, 2H), 6.79 (s, 2H), 6.53-6.23 (m, 2H), 6.12-5.96 (m, 1H), 5.93-5.55 (m, 2H), 5.55-5.27 (m, 1H), 5.16-4.90 (m, 3H), 4.75-4.57 (m, 4H), 4.55-4.33 (m, 4H), 4.24-4.13 (m, 1H), 4.15-3.89 (m, 4H), 3.83-3.53 (m, 1H), 3.53-3.42 (m, 2H), 2.82 (s, 3H), 2.78-2.64 (m, 1H), 2.37-2.23 (m, 2H), 2.23-1.99 (m, 1H), 1.73-1.52 (m, 4H), 1.52-1.42 (m, 3H), 1.40-1.20 (m, 4H), 1.09-0.85 (m, 7H), 0.72-0.59 (m, 1H).

### 5. Synthesis of LP5

The synthesis of LP5 is shown below:

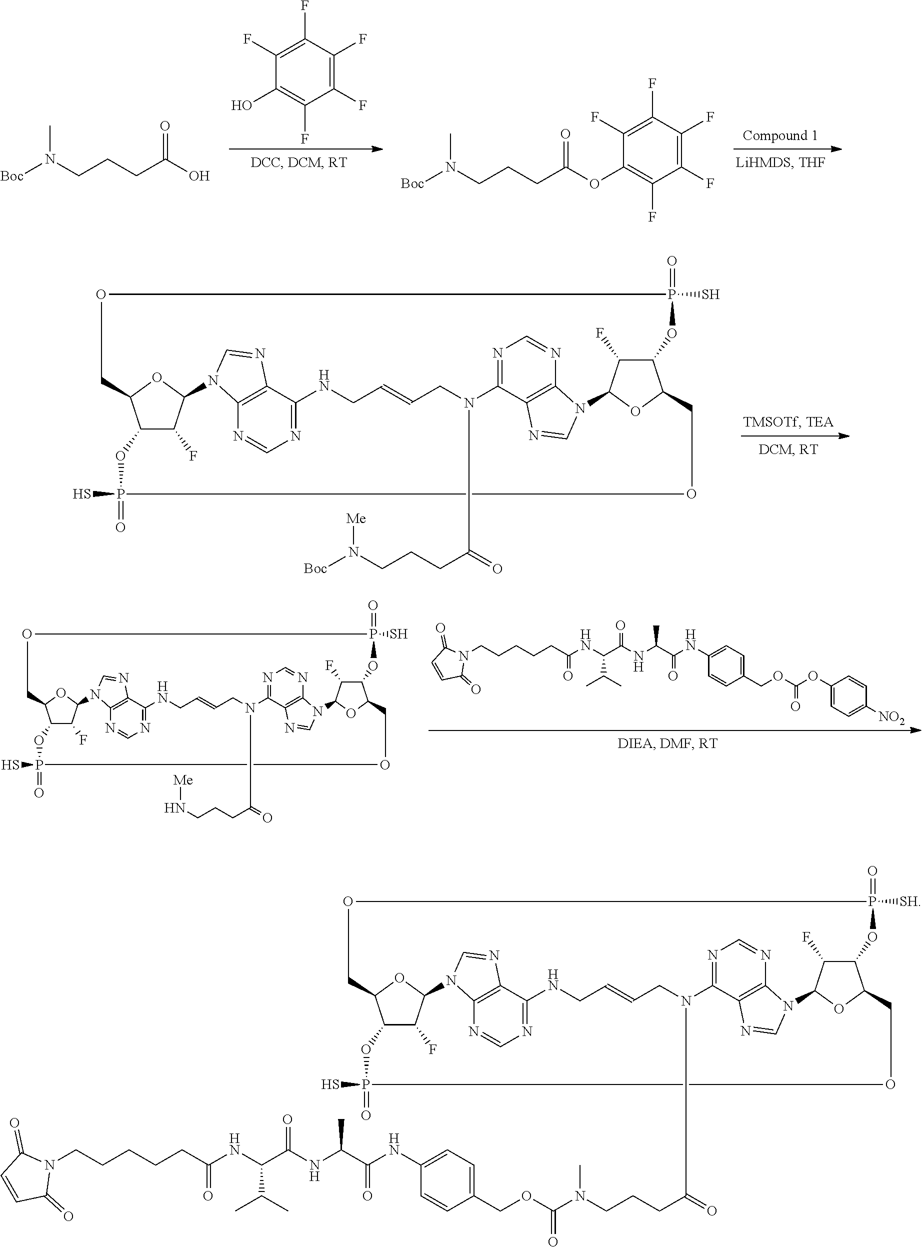

### Synthesis of 2,3,4,5,6-pentafluorophenyl 4-[(tert-butoxycarbonyl)(methyl)amino]butanoate

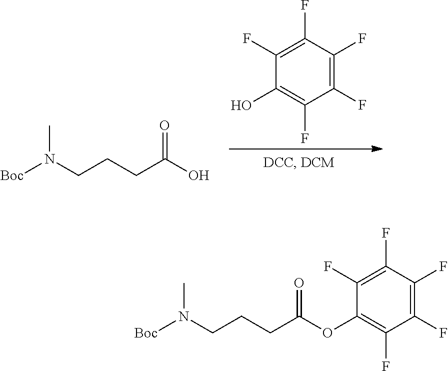 [SMILES: CN(CCCC(=O)O)C(=O)OC(C)(C)C ; Oc1c(F)c(F)c(F)c(F)c1F ; CN(CCCC(=O)Oc1c(F)c(F)c(F)c(F)c1F)C(=O)OC(C)(C)C]

DCC (1.90 g, 9.206 mmol) was added to a mixture of 4-[(tert-butoxycarbonyl)(methyl)amino]butanoic acid (1 g, 4.603 mmol) and pentafluorophenol (847.19 mg, 4.603 mmol) in DCM (15 mL). The resulting mixture was stirred at 25° C. for 2 h. The resulting mixture was concentrated under reduced pressure. The residue was purified by silica gel column chromatography, eluted with PE/EtOAc (7:1). This resulted in 1.5 g of 2,3,4,5,6-pentafluorophenyl 4-[(tert-butoxycarbonyl)(methyl)amino]butanoate as a white solid. LC-MS (ESI): 384 [M+H]⁺.

### Synthesis of tert-butyl N-{4-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-13-yl]-4-oxobutyl}-N-methylcarbamate

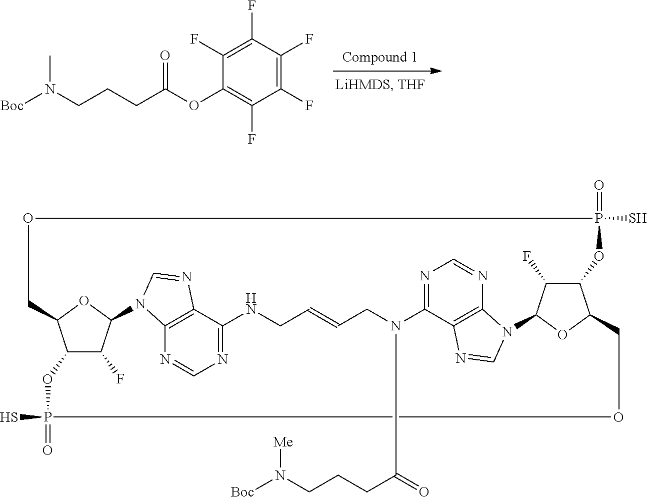 [SMILES: CN(CCCC(=O)Oc1c(F)c(F)c(F)c(F)c1F)C(=O)OC(C)(C)C ; CN(CCCC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32)C(=O)OC(C)(C)C]

LiHMDS (0.268 mmol) was added to a mixture of Compound 1 (50 mg, 0.067 mmol) in THF (2 mL) at −78° C. The resulting mixture was stirred at −78° C. for 30 min. To the above mixture was added 2,3,4,5,6-pentafluorophenyl 4-[(tert-butoxycarbonyl)(methyl)amino]butanoate (25 mg, 0.067 mmol) dropwise at −78° C. The resulting mixture was slowly warmed to 0° C. over 1 h. To the above mixture was added LiHMDS (0.134 mmol) at −78° C. and stirred for 5 min. To the mixture was added 2,3,4,5,6-pentafluorophenyl 4-[(tert-butoxycarbonyl)(methyl)amino]butanoate (25 mg, 0.067 mmol) dropwise at −78° C. The resulting mixture was slowly warmed to 0° C. over 1 h. The reaction was quenched with AcOH. The resulting mixture was concentrated under reduced pressure. The residue was purified by Prep-TLC (DCM/MeOH=7:1). This resulted in 40 mg of tert-butyl N-{4-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-13-yl]-4-oxobutyl}-N-methylcarbamate as a yellow solid. LC-MS (ESI): 946 [M+H]⁺.

### Synthesis of (1R,3R,15E,28R,29R,30R,31R,34S,36R,39R,41R)-29,41-difluoro-13-[4-(methylamino)butanoyl]-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-34,39-dione

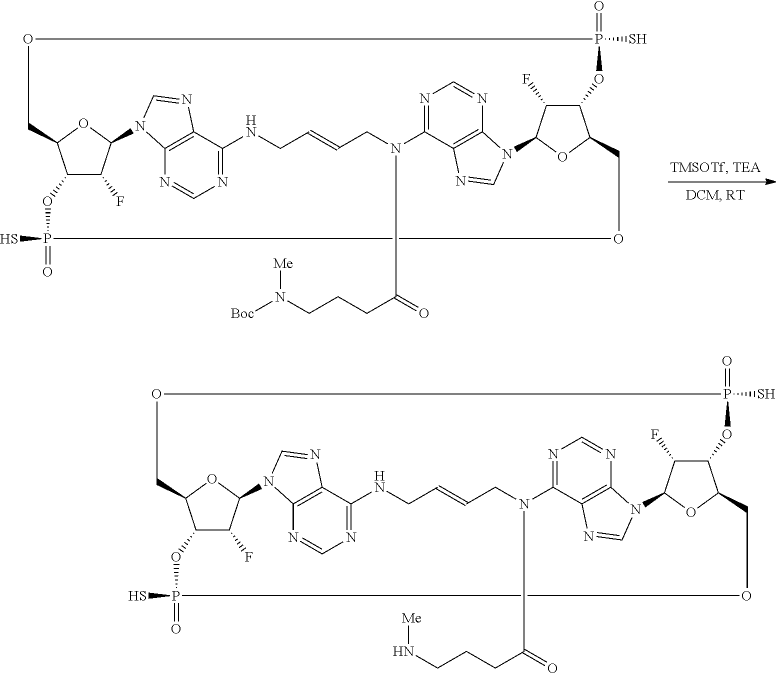 [SMILES: CN(CCCC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32)C(=O)OC(C)(C)C ; CNCCCC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32]

TMSOTf (41 mg, 0.185 mmol) was added to a mixture of tert-butyl N-{4-[(1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-13-yl]-4-oxobutyl}-N-methylcarbamate (35 mg, 0.037 mmol) and TEA (11 mg, 0.111 mmol) in DCM (2 mL). The resulting mixture was stirred at room temperature for 30 min. The resulting mixture was concentrated under reduced pressure. This resulted in crude of (1R,3R,15E,28R,29R,30R,31R,34,36R,39R,41R)-29,41-difluoro-13-[4-(methylamino)butanoyl]-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-34,39-dione as a yellow oil. LC-MS (ESI): 846 [M+H]⁺.

### Synthesis of {4-[(2S)-2-[(2S)-2-[6-(2,5-dioxo-2,5-dihydro-1H-pyrrol-1yl)hexanamido]-3-methylbutanamido]propanamido]phenyl}methyl N-{4-[(1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-13-yl]-4-oxobutyl}-N-methylcarbamate (LP5)

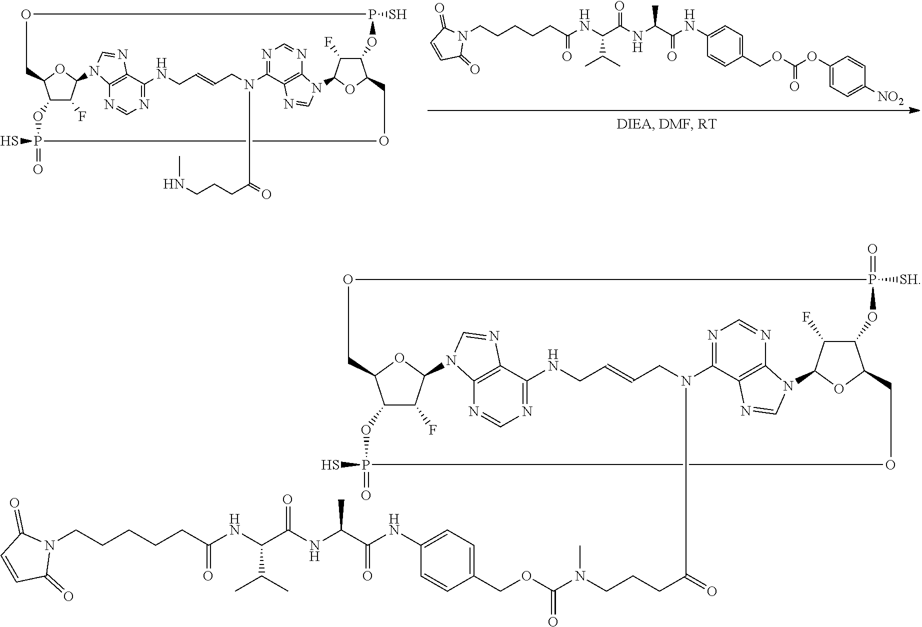 [SMILES: CNCCCC(=O)N1C/C=C/CNc2ncnc3c2ncn3[C@@H]2O[C@@H]3CO[P@](S)O[C@H]4[C@@H](F)[C@@H](O[C@@H]4CO[P@@](=O)(S)O[C@H]3[C@H]2F)n2cnc3c1ncnc32 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](C)C(=O)Nc1ccc(COC(=O)N(C)CCCC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

DIEA (18.34 mg, 0.140 mmol) were added to a mixture of (1R,3R,15E,28R,29R,30R,31R,34,36R,39R,41R)-29,41-difluoro-13-[4-(methylamino)butanoyl]-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-34,39-dione (30 mg, 0.035 mmol) and {4-[(2S)-2-[(2S)-2-[6-(2,5-dioxopyrrol-1-yl)hexanamido]-3-methylbutanamido]propanamido]phenyl}methyl 4-nitrophenyl carbonate (23.12 mg, 0.035 mmol) in DMF (1 mL). The resulting mixture was stirred at 25° C. for 2 h. The resulting mixture was concentrated under reduced pressure. The residue was purified by silica gel column chromatography, eluted with Prep-TLC ((1% TEA) CH₂Cl₂/MeOH 7:1) and Prep-HPLC Column: XBridge Prep Phenyl OBD Column, 19×250 mm, 5 μm; Mobile Phase A: Water(50 mmol HCO₂NH₄), Mobile Phase B: MeCN; Flow rate: 25 mL/min; Gradient: 25% B to 40% B in 10 min, 40% B; Wavelength: 254 nm. This resulted in 10.1 mg of {4-[(2S)-2-[(2S)-2-[6-(2,5-dioxo-2,5-dihydro-1H-pyrrol-1yl)hexanamido]-3-methylbutanamido]propanamido]phenyl}methyl N-{4-[(1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-13-yl]-4-oxobutyl}-N-methylcarbamate (LP5) as a white solid.

LC-MS (ESI): 1358.50 [M+H]⁺.

¹H NMR (400 MHz, Methanol-d₄) δ 9.02-8.62 (m, 3H), 8.19 (s, 1H), 7.56 (m, 2H), 7.36-7.16 (m, 2H), 6.79 (s, 2H), 6.52-6.30 (m, 2H), 6.11-5.76 (m, 1H), 5.73-5.44 (m, 2H), 5.08-4.95 (m, 2H), 4.72-4.41 (m, 9H), 4.20-3.92 (m, 3H), 3.75-3.40 (m, 4H), 3.28-3.16 (m, 4H), 2.81 (s, 3H), 2.70-2.55 (m, 2H), 2.49-2.03 (m, 4H), 1.93-1.77 (m, 2H), 1.68-1.60 (m, 3H), 1.59-1.50 (m, 2H), 1.48-1.41 (m, 4H), 1.36-1.25 (m, 4H), 1.03-0.95 (m, 6H).

### 6. Synthesis of LP6

The synthesis of LP6 is shown below:

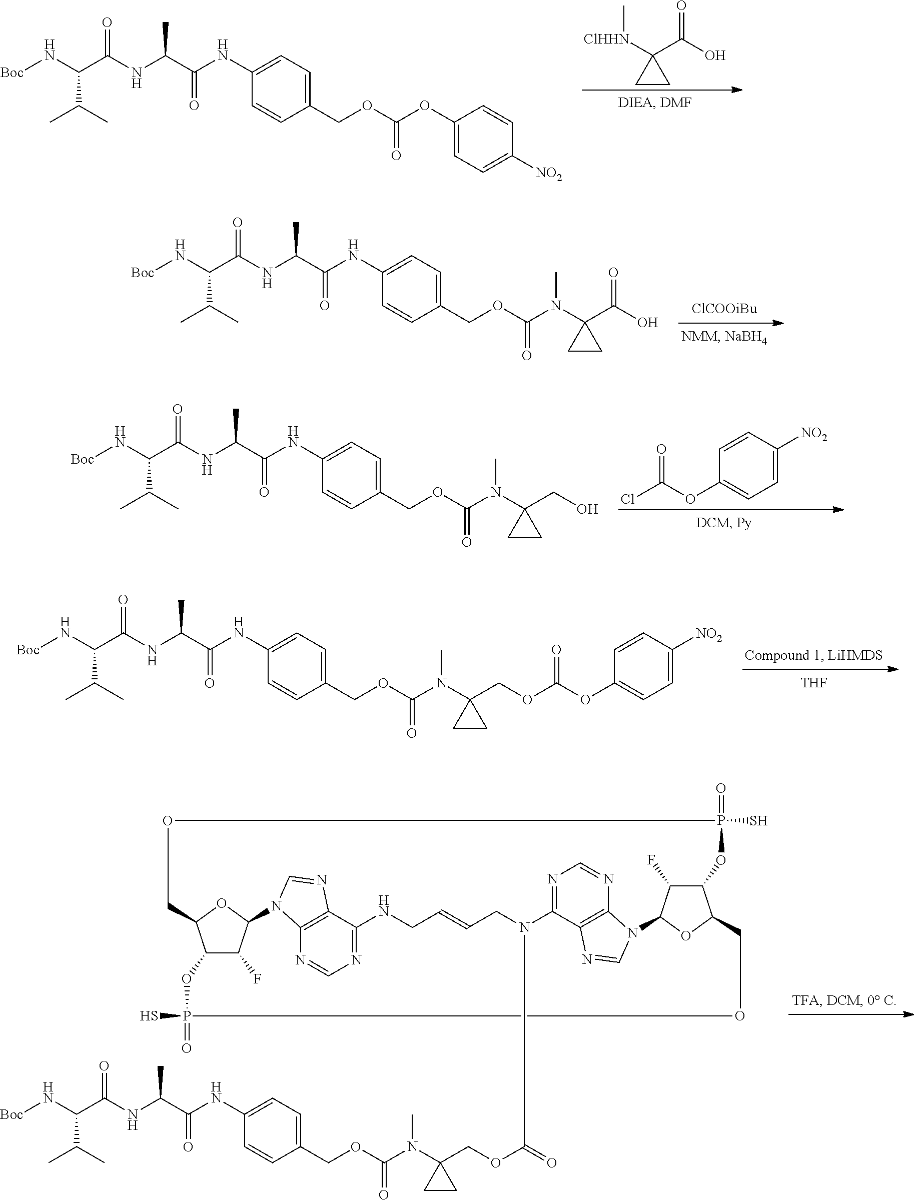

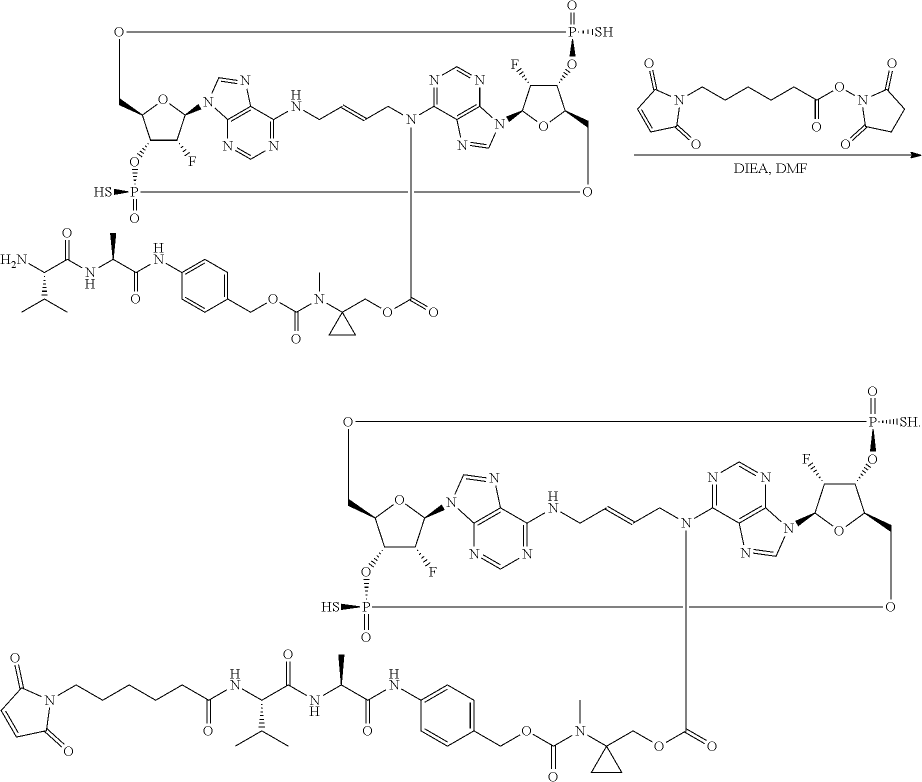 [SMILES: CC(C)[C@H](N)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

### Synthesis of 1-{[({4-[(2S)-2-[(2S)-2-{[(tert-butoxy)carbonyl]amino}-3-methylbutanamido]propanamido]phenyl}methoxy)carbonyl](methyl)amino}cyclopropane-1-carboxylic acid

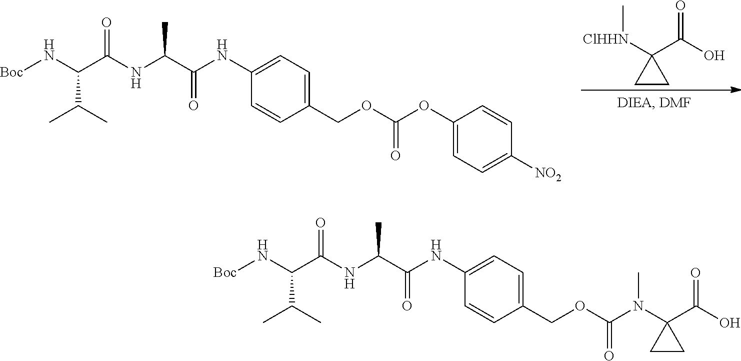

DIEA (231.38 mg, 1.790 mmol) and 1-(methylamino)cyclopropane-1-carboxylic acid hydrochloride (136 mg, 0.895 mmol) were added to a mixture of {4-[(2S)-2-[(2S)-2-[(tert-butoxycarbonyl)amino]-3-methylbutanamnido]propanamido]phenyl}methyl 4-nitrophenyl carbonate (500 mg, 0.895 mmol) in DMF (5 mL). The resulting mixture was stirred at 60° C. for 14 h. The resulting mixture was concentrated under reduced pressure. The residue was purified by silica gel column chromatography, eluted with DCM/MeOH (10:1). This resulted in 350 mg of 1-{[({4-[(2S)-2-[(2S)-2-{[(tert-butoxy)carbonyl]amino}-3-methylbutanamido]propanamido]phenyl}methoxy)carbonyl](methyl)amino}cyclopropane-1-carboxylic acid as a white solid. LC-MS (ESI): 535 [M+H]⁺.

### Synthesis of {4-[(2S)-2-[(2S)-2-{[(tert-butoxy)carbonyl]amino}-3-methylbutanamido]propanamido]phenyl}methyl N-[1-(hydroxymethyl)cyclopropyl]-N-methylcarbamate

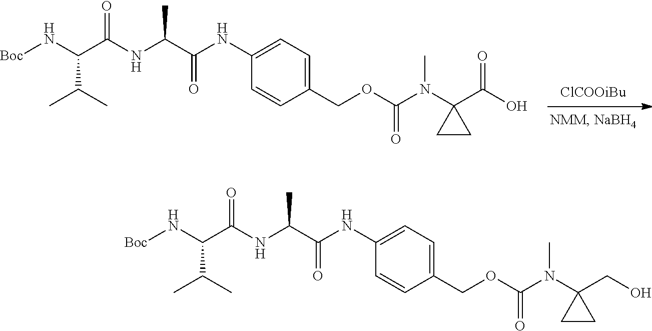 [SMILES: CC(C)[C@H](NC(=O)OC(C)(C)C)C(=O)N[C@@H](C)C(=O)Nc1ccc(COC(=O)N(C)C2(C(=O)O)CC2)cc1 ; CC(C)[C@H](NC(=O)OC(C)(C)C)C(=O)N[C@@H](C)C(=O)Nc1ccc(COC(=O)N(C)C2(CO)CC2)cc1 ; "ClCOOiBu"]

2-methylpropyl carbonochloridate (126 mg, 0.925 mmol) and 4-methylmorpholine (94 mg, 0.925 mmol) were added to a mixture of 1-{[({4-[(2S)-2-[(2S)-2-{[(tert-butoxy)carbonyl]amino}-3-methylbutanamido]propanamido]phenyl}methoxy)carbonyl](methyl)amino}cyclopropane-1-carboxylic acid (330 mg, 0.617 mmol) in DME (5 mL) at 0° C. The resulting mixture was stirred at 0° C. for 20 min. To the above mixture was added NaBH₄ (47 mg, 1.234 mmol) in H₂O (1 mL) dropwise. The resulting mixture was stirred at 25° C. for 2 h. The resulting mixture was concentrated under reduced pressure. The residue was purified by silica gel column chromatography, eluted with DCM/MeOH (10:1). This resulted in 260 mg of {4-[(2S)-2-[(2S)-2-{[(tert-butoxy)carbonyl]amino}-3-methylbutanamido]propanamido]phenyl}methyl N-[1-(hydroxymethyl)cyclopropyl]-N-methylcarbamate as a white solid. LC-MS (ESI): 521 [M+H]⁺.

### Synthesis of (1-{[({4-[(2S)-2-[(2S)-2-{[(tert-butoxy)carbonyl]amino}-3-methylbutanamido]propanamido]phenyl}methoxy)carbonyl](methyl)amino}cyclopropyl)methyl 4-nitrophenyl carbonate

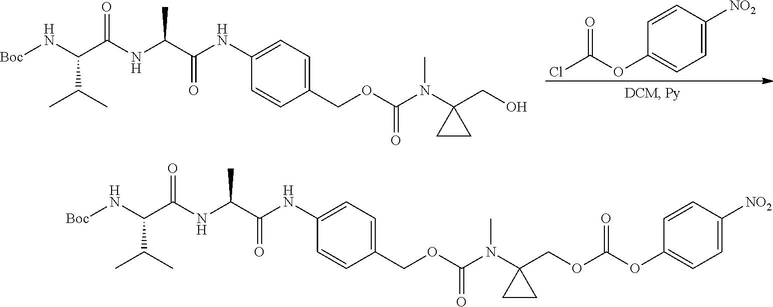 [SMILES: CC(C)[C@H](NC(=O)OC(C)(C)C)C(=O)N[C@@H](C)C(=O)Nc1ccc(COC(=O)N(C)C2(CO)CC2)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N(C)C2(COC(=O)Oc3ccc([N+](=O)[O-])cc3)CC2)cc1]

4-nitrophenyl carbonochloridate (140 mg, 0.692 mmol) and Pyridine (55 mg, 0.692 mmol) were added to a mixture of {4-[(2S)-2-[(2S)-2-{[(tert-butoxy)carbonyl]amino}-3-methylbutanamido]propanamido]phenyl}methyl N-[1-(hydroxymethyl)cyclopropyl]-N-methylcarbamate (240 mg, 0.461 mmol) in DCM (5 mL). The resulting mixture was stirred at 25° C. for 4 h. The resulting mixture was concentrated under reduced pressure. The residue was purified by silica gel column chromatography, eluted with PE/EtOAc (1:2). This resulted in 250 mg of (1-{[({4-[(2S)-2-[(2S)-2-{[(tert-butoxy)carbonyl]amino}-3-methylbutanamido]propanamido]phenyl}methoxy)carbonyl](methyl)amino}cyclopropyl)methyl 4-nitrophenyl carbonate as a white solid. LC-MS (ESI): 686 [M+H]⁺.

### Synthesis of: (1-{[({4-[(2S)-2-[(2S)-2-{[(tert-butoxy)carbonyl]amino}-3-methylbutanamido]propanamido]phenyl}methoxy)carbonyl](methyl)amino}cyclopropyl)methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

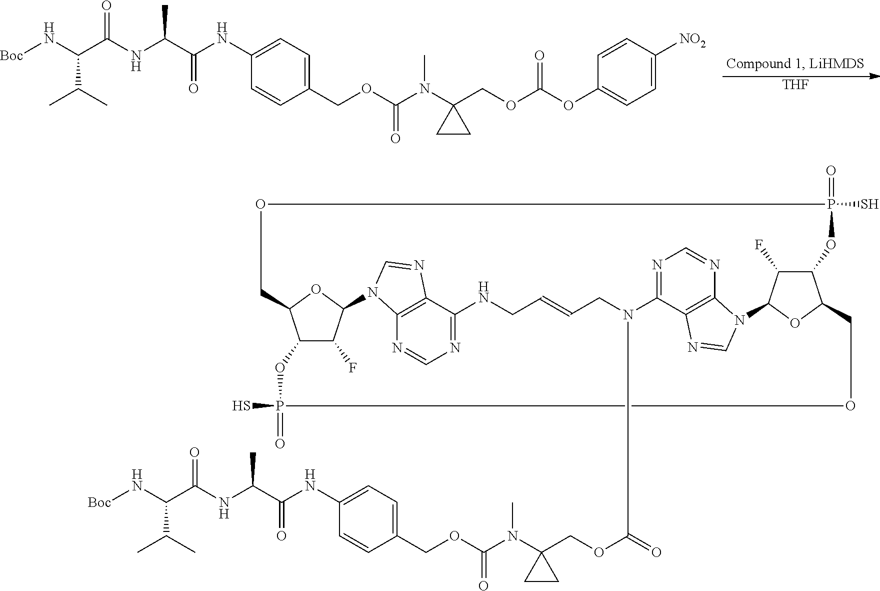 [SMILES: CC(C)[C@H](NC(=O)OC(C)(C)C)C(=O)N[C@@H](C)C(=O)Nc1ccc(COC(=O)N(C)C2(COC(=O)Oc3ccc([N+](=O)[O-])cc3)CC2)cc1 ; CC(C)[C@H](NC(=O)OC(C)(C)C)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; "THF"]

LiHMDS (0.75 mL 0.752 mmol) was added to a mixture of Compound 1 (70 mg, 0.094 mmol) in THF (5 mL) at −78° C. The resulting mixture was stirred at −78° C. for 20 min. To the above mixture was added (1-{[({4-[(2S)-2-[(2S)-2-[(tert-butoxycarbonyl)amino]-3-methylbutanamido]propanamido]phenyl}methoxy)carbonyl](methyl)amino}cyclopropyl)methyl 4-nitrophenyl carbonate (65 mg, 0.094 mmol) dropwise at −78° C. The resulting mixture was stirred at−78° C. for 2 h. The reaction was quenched by the addition of AcOH. The resulting mixture was concentrated under reduced pressure. The residue was purified by Prep-TLC (DCM/MeOH 7:1). This resulted in 55 mg of (1-{[({4-[(2S)-2-[(2S)-2-{[(tert-butoxy)carbonyl]amino}-3-methylbutanamido]propanamido]phenyl}methoxy)carbonyl](methyl)amino}cyclopropyl)methyl(1R,3R,15,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a white solid. LC-MS (ESI): 1293 [M+H]⁺.

### Synthesis of (1-{[({4-[(2S)-2-[(2S)-2-amino-3-methylbutanamido]propanamido]phenyl}methoxy)carbonyl](methyl)amino}cyclopropyl)methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

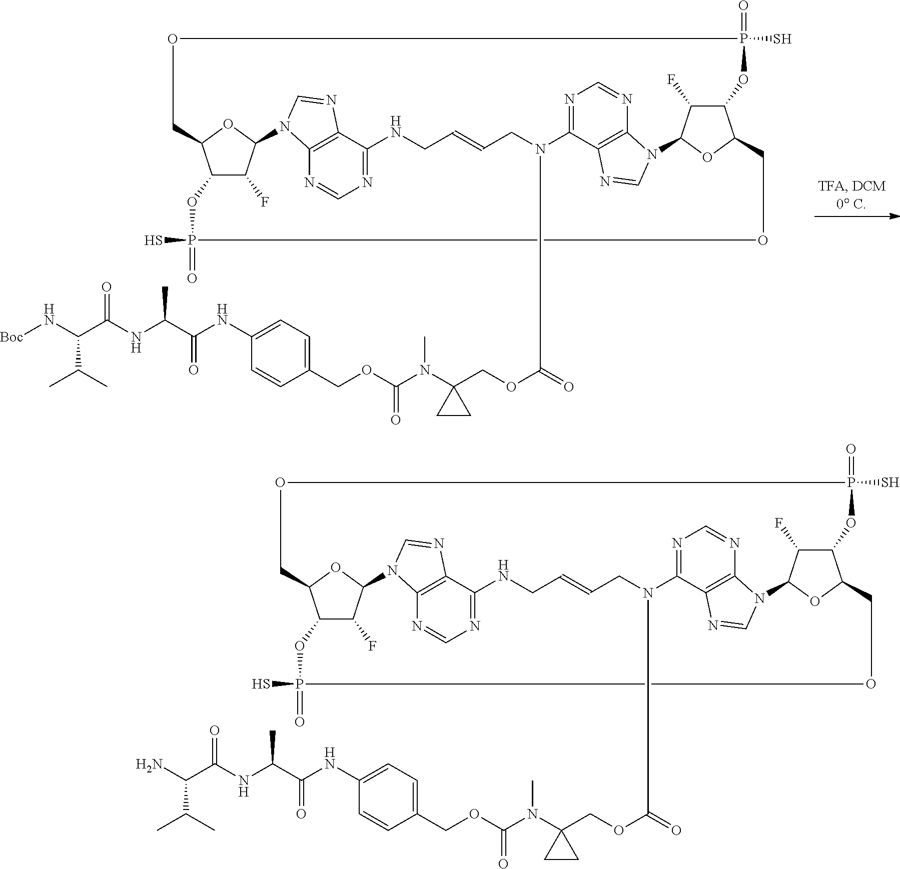 [SMILES: CC(C)[C@H](NC(=O)OC(C)(C)C)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; CC(C)[C@H](N)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

TFA (0.5 mL) was added to a mixture of (1-{[({4-[(2S)-2-[(2S)-2-[(tert-butoxycarbonyl)amino]-3-methylbutanamido]propanamido]phenyl}methoxy)carbonyl](methyl)amino}cyclopropyl)methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (50 mg, 0.039 mmol) in DCM (3 mL). The resulting mixture was stirred at 0° C. for 1 h. The resulting mixture was concentrated under reduced pressure. This resulted in crude of (1-{[({4-[(2S)-2-[(2S)-2-amino-3-methylbutanamido]propanamido]phenyl}methoxy)carbonyl](methyl)amino}cyclopropyl)methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacycl[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as an oil. LC-MS (ESI): 1193[M+H]⁺.

### Synthesis of (1-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}cyclopropyl)methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP6)

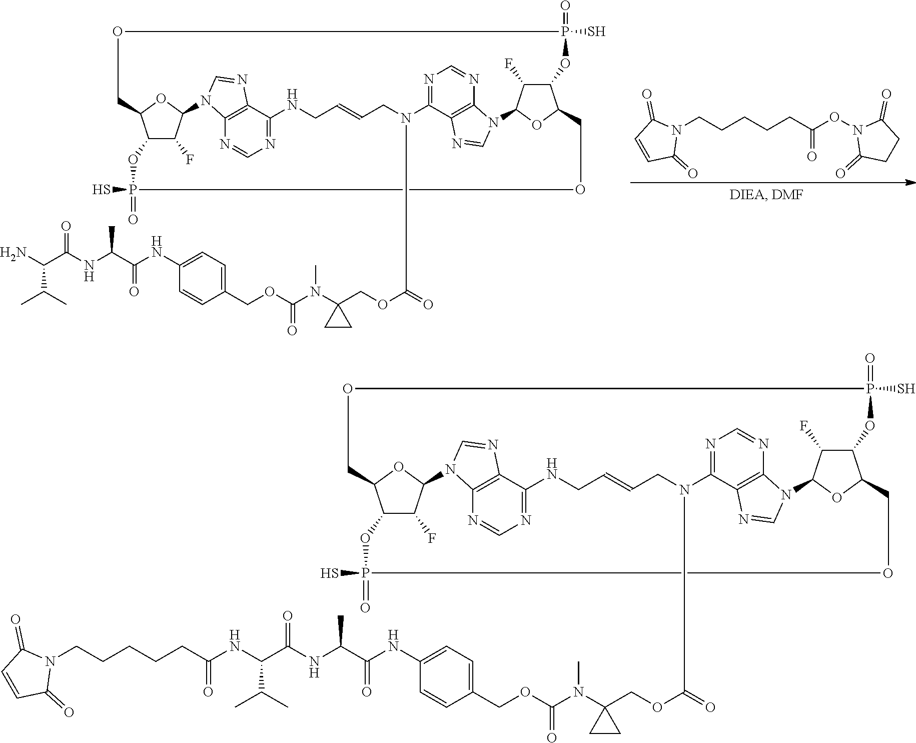 [SMILES: CC(C)[C@H](N)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)C2(COC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

2,5-dioxopyrrolidin-1-yl 6-(2,5-dioxopyrrol-1-yl)hexanoate (21 mg, 0.068 mmol) and DIEA (13 mg, 0.102 mmol) were added to a mixture of (1-{[({4-[(2S)-2-[(2S)-2-amino-3-methylbutanamido]propanamido]phenyl}methoxy)carbonyl](methyl)amino}cyclopropyl)methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg, 0.034 mmol) in DMF (1.5 mL) at 25° C. The resulting mixture was stirred at 25° C. for 1 h. The resulting mixture was concentrated under reduced pressure. The residue was purified by silica gel column chromatography, eluted with Prep-TLC (DCM/MeOH 5:1). The crude product was purified by Prep-HPLC with the following conditions Column: XBridge Prep Phenyl OBD Column, 19×250 mm, 5 μm; Mobile Phase A: Water(50 mmol HCO₂NH₄), Mobile Phase B: MeCN; Flow rate: 25 mL/min; Gradient: 25% B to 40% B in 10 min; Wavelength: 254 nm. This resulted in 16.3 mg of (1-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}cyclopropyl)methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP6) as a white solid.

LC-MS (ESI): 1386.50 [M+H]⁺.

¹H NMR (300 MHz, Methanol-d₄) δ 9.03-8.63 (m, 3H), 8.28-7.98 (m, 2H), 7.57 (d, J=7.4 Hz, 2H), 7.41-7.11 (m, 2H), 6.80 (s, 2H), 6.58-6.21 (m, 2H), 6.10-5.25 (m, 4H), 4.73-4.35 (m, 9H), 4.29-3.87 (m, 6H), 3.83-3.41 (m, 5H), 2.82-2.51 (m, 3H), 2.36-2.19 (m, 2H), 2.17-1.99 (m, 1H), 1.70-1.50 (m, 3H), 1.51-1.17 (m, 9H), 1.03-0.93 (m, 6H), 0.91-0.80 (m, 6H).

### 7. Synthesis of LP7

The synthesis of LP7 is shown below:

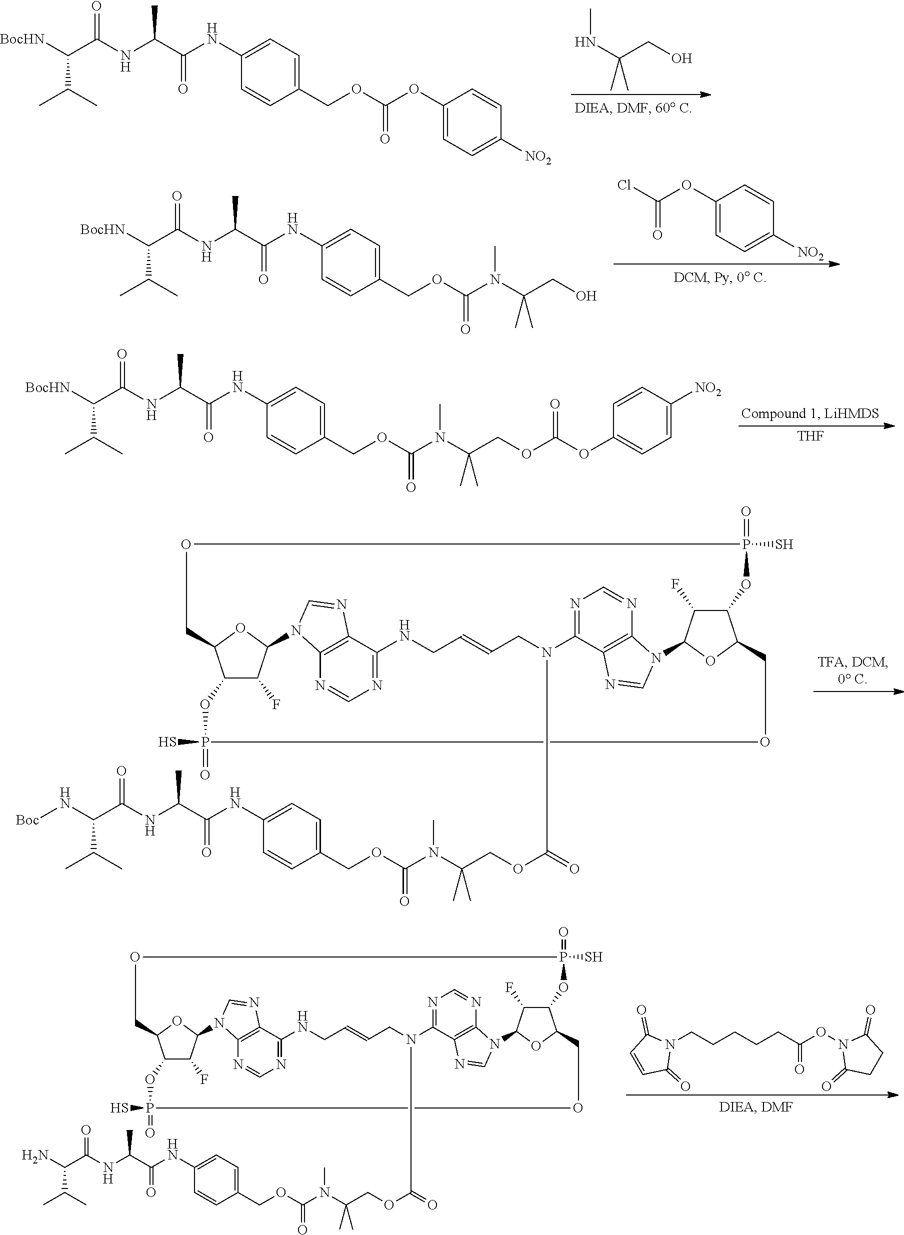

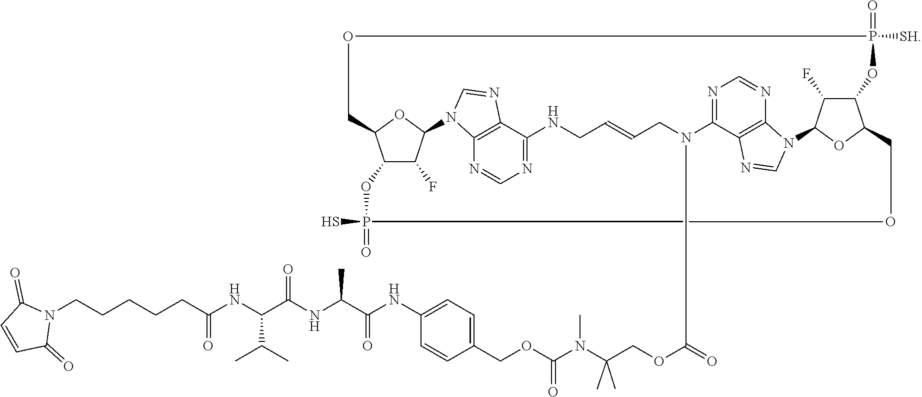 [SMILES: CC(C)[C@H](NC(=O)CCCCCN1C(=O)C=CC1=O)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of tert-butyl N-[(1S)-1-{[(1S)-1-{[4-({[(1-hydroxy-2-methylpropan-2-yl)(methyl)carbamoyl]oxy}methyl)phenyl]carbamoyl}ethyl]carbamoyl}-2-methylpropyl]carbamate

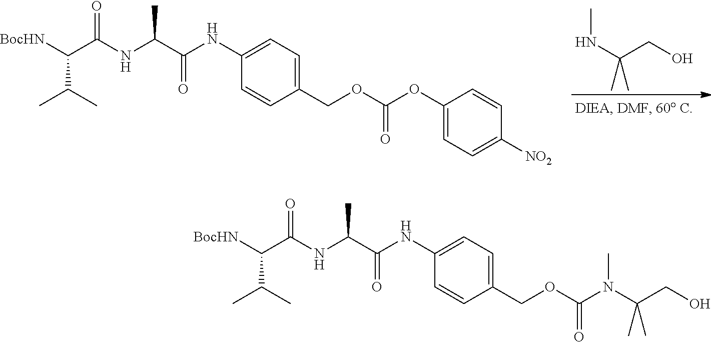

2-methyl-2-(methylamino)propan-1-ol (185 mg, 1.790 mmol) and DIEA (463 mg, 3.580 mmol) were added to a mixture of {4-[(2S)-2-[(2S)-2-[(tert-butoxycarbonyl)amino]-3-methylbutanamido]propanamido]phenyl}methyl 4-nitrophenyl carbonate (1 g, 1.790 mmol) in DMF (5 mL). The resulting mixture was stirred at 60° C. for 14 h. The resulting mixture was concentrated under reduced pressure. The residue was purified by silica gel column chromatography, eluted with DCM/MeOH (10:1). This resulted in 560 mg of tert-butyl N-[(1S)-1-{[(1S)-1-{[4-({[(1-hydroxy-2-methylpropan-2-yl)(methyl)carbamoyl]oxy}methyl)phenyl]carbamoyl}ethyl]carbamoyl}-2-methylpropyl]carbamate as a white solid. LC-MS (ESI): 523 [M+H]⁺.

### Synthesis of tert-butyl N-[(1S)-2-methyl-1-{[(1S)-1-{[4-({[methyl({2-methyl-1-[(4-nitrophenoxycarbonyl)oxy]propan-2-yl})carbamoyl]oxy}methyl)phenyl]carbamoyl}ethyl]carbamoyl}propyl]carbamate

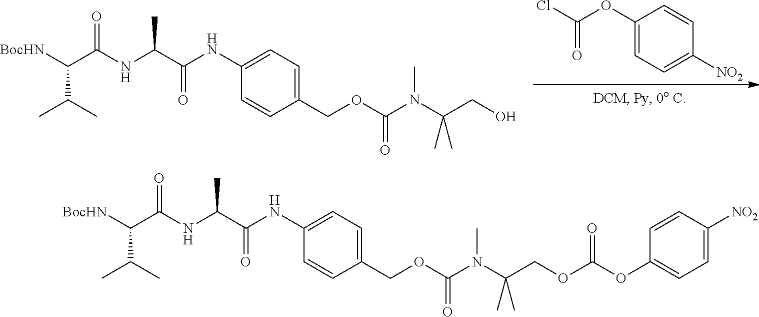 [SMILES: CC(C)[C@H](NC(=O)OC(C)(C)C)C(=O)N[C@@H](C)C(=O)Nc1ccc(COC(=O)N(C)C(C)(C)CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N(C)C(C)(C)COC(=O)Oc2ccc([N+](=O)[O-])cc2)cc1]

4-nitrophenyl carbonochloridate (313 mg, 1.549 mmol) and Pyridine (123 mg, 1.549 mmol) were added to a solution of tert-butyl N-[(1S)-1-{[(1S)-1-{[4-({[(1-hydroxy-2-methylpropan-2-yl)(methyl)carbamoyl]oxy}methyl)phenyl]carbamoyl}ethyl]carbamoyl}-2-methylpropyl]carbamate (540 mg, 1.033 mmol) in DCM (5 mL). The resulting mixture was stirred at 25° C. for 4 h under nitrogen atmosphere. The resulting mixture was concentrated under reduced pressure. The residue was purified by silica gel column chromatography, eluted with PE/EtOAc (1:2). This resulted in 630 mg of tert-butyl N-[(1S)-2-methyl-1-{[(1S)-1-{[4-({[methyl({2-methyl-1-[(4-nitrophenoxycarbonyl)oxy]propan-2-yl})carbamoyl]oxy}methyl)phenyl]carbamoyl}ethyl]carbamoyl}propyl]carbamate as a white solid. LC-MS (ESI): 688 [M+H]⁺.

### Synthesis of 2-{[({4-[(2S)-2-[(2S)-2-{[(tert-butoxy)carbonyl]amino}-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

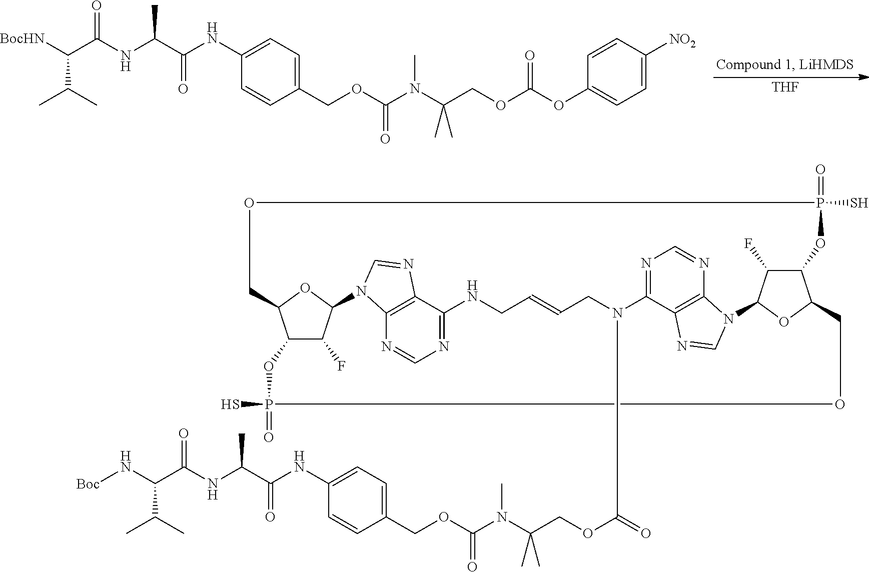 [SMILES: "THF" ; CC(C)[C@H](NC(=O)OC(C)(C)C)C(=O)N[C@@H](C)C(=O)Nc1ccc(COC(=O)N(C)C(C)(C)COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

LiHMDS (0.75 mL, 0.752 mmol) was added to a mixture of Compound 1 (70 mg, 0.094 mmol) in THF (3 mL). The resulting mixture was stirred at −78° C. for 30 min under nitrogen atmosphere. To the above mixture was added tert-butyl N-[(1S)-2-methyl-1-{[(1S)-1-{[4-({[methyl({2-methyl-1-[(4-nitrophenoxycarbonyl)oxy]propan-2-yl})carbamoyl]oxy}methyl)phenyl]carbamoyl}ethyl]carbamoyl}propyl]carbamate (65 mg, 0.094 mmol) dropwise at −78° C. The resulting mixture was stirred at −78° C. for 2 h under nitrogen atmosphere. The reaction was quenched by the addition of AcOH. The resulting mixture was concentrated under reduced pressure. The residue was purified by Prep-TLC (DCM/MeOH 7:1). This resulted in 45 mg of 2-{[({4-[(2S)-2-[(2S)-2-{[(tert-butoxy)carbonyl]amino}-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a white solid. LC-MS (ESI): 1295 [M+H]⁺.

### Synthesis of 2-{[({4-[(2S)-2-[(2S)-2-amino-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

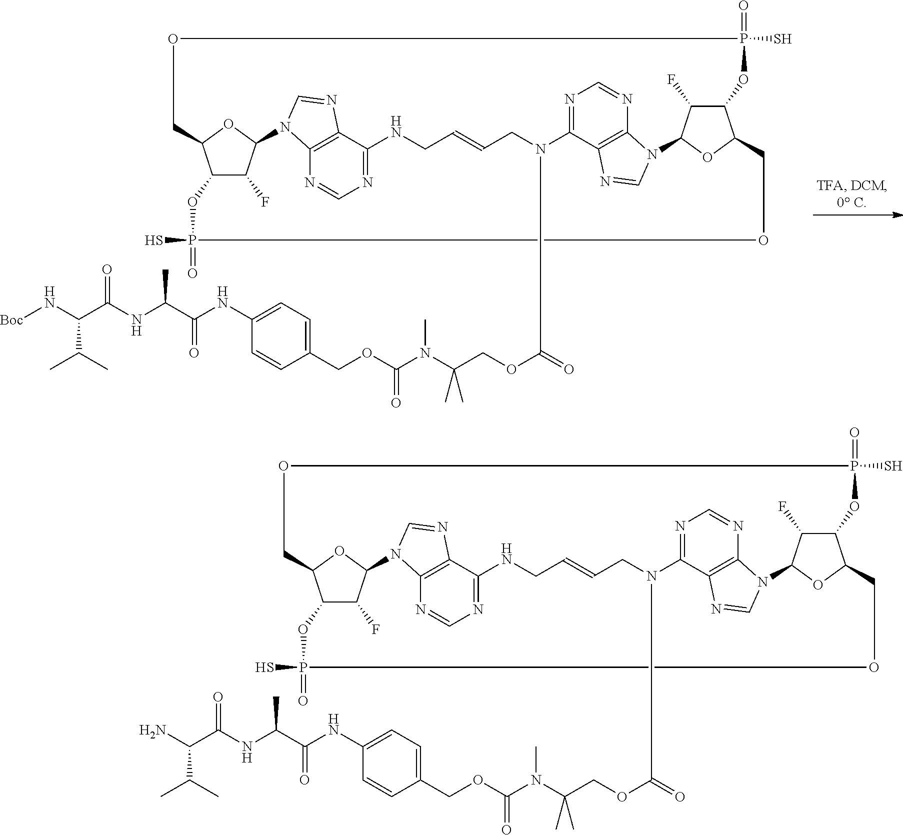 [SMILES: CC(C)[C@H](NC(=O)OC(C)(C)C)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

TFA (0.5 mL) was added to a mixture of 2-{[({4-[(2S)-2-[(2S)-2-{[(tert-butoxy)carbonyl]amino}-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (35 mg, 0.027 mmol) in DCM (3 mL). The resulting mixture was stirred at 0° C. for 1 h, The resulting mixture was concentrated under reduced pressure. This resulted in 30 mg of 2-{[({4-[(2S)-2-[(2S)-2-amino-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as an oil. LC-MS (ESI): 1195 [M+H]⁺.

### Synthesis of 2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}-2-methylpropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP7)

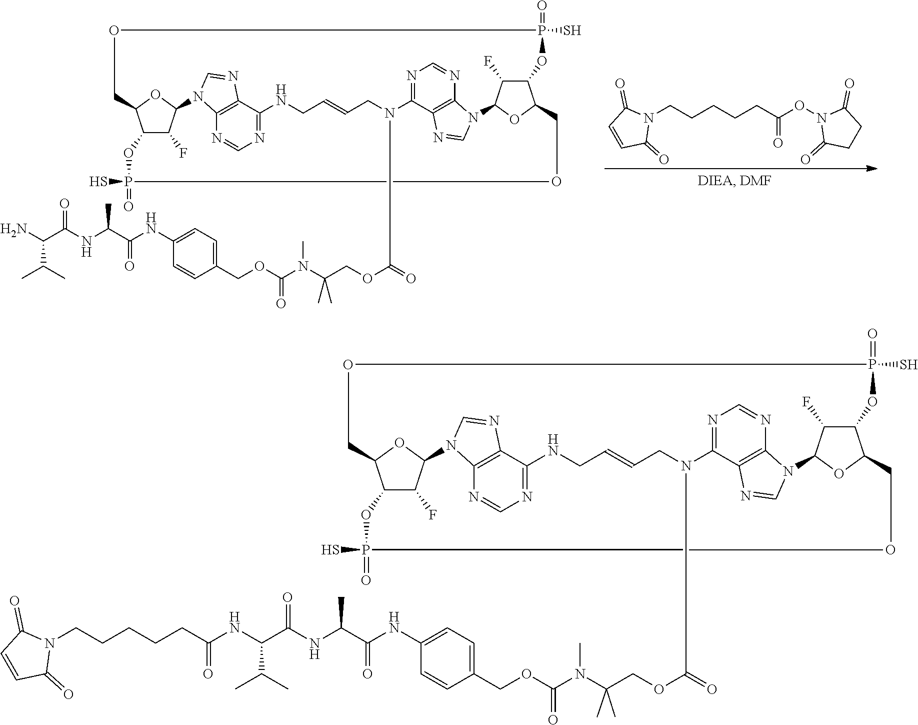 [SMILES: CC(C)[C@H](N)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)C(C)(C)COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

2,5-dioxopyrrolidin-1-yl 6-(2,5-dioxopyrrol-1-yl)hexanoate (15 mg, 0.050 mmol) and DIEA (10 mg, 0.075 mmol) were added to a mixture of 2-{[({4-[(2S)-2-[(2S)-2-amino-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (30 mg, 0.025 mmol) in DMF (2 mL). The resulting mixture was stirred at 25° C. for 1 h. The resulting mixture was concentrated under reduced pressure. The residue was purified by silica gel column chromatography, eluted with Prep-TLC (DCM/MeOH 5:1). The crude product was purified by Prep-HPLC with the following conditions Column: XBridge Prep Phenyl OBD Column, 19×250 mm, 5 μm; Mobile Phase A: Water(50 mmol HCO₂NH₄), Mobile Phase B: MeCN; Flow rate: 25 mL/min; Gradient: 25% B to 40% B in 15 min, 40% B; Wavelength: 254 nm. This resulted in 10 mg of 2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}-2-methylpropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP7) as a white solid.

LC-MS (ESI): 1388.55[M+H]⁺.

¹H NMR (300 MHz, Methanol-d₄) δ 9.01-8.70 (m, 3H), 8.33-8.26 (m, 2H), 8.11-8.01 (m, 2H), 7.62 (d, J=8.0 Hz, 2H), 7.27 (d, J=8.1 Hz, 2H), 6.84-6.76 (m, 2H), 6.58-6.24 (m, 2H), 6.10-5.30 (m, 3H), 5.13-4.98 (m, 3H), 4.82-4.59 (m, 5H), 4.56-4.14 (m, 8H), 4.13-3.89 (m, 2H), 3.74-3.54 (m, 1H), 3.51-3.42 (m, 3H), 2.78-2.62 (m, 3H), 2.39-2.26 (m, 2H), 2.23-2.10 (m, 1H), 1.70-1.40 (m, 8H), 1.36-1.22 (m, 9H), 1.03-0.93 (m, 6H).

### 8. Synthesis of LP8

The synthesis of LP8 is shown below:

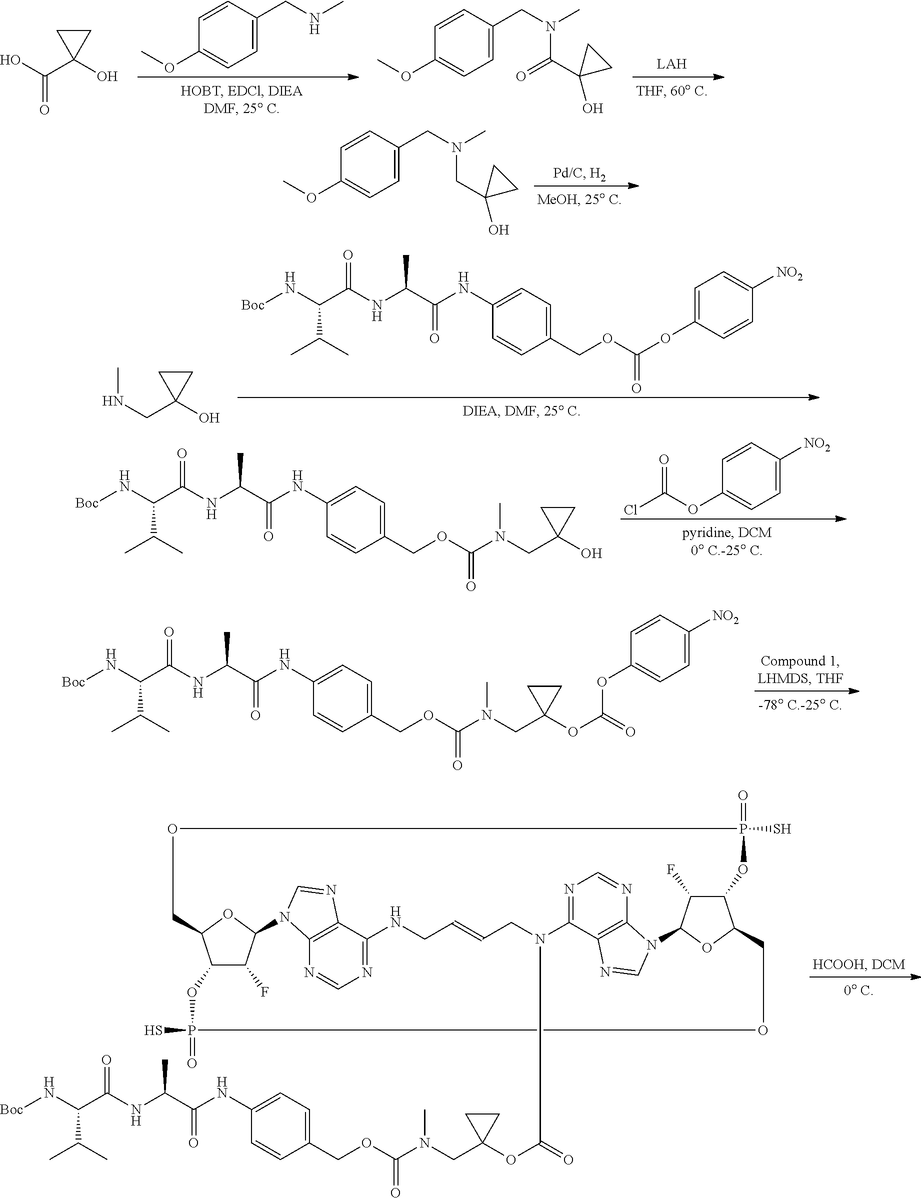

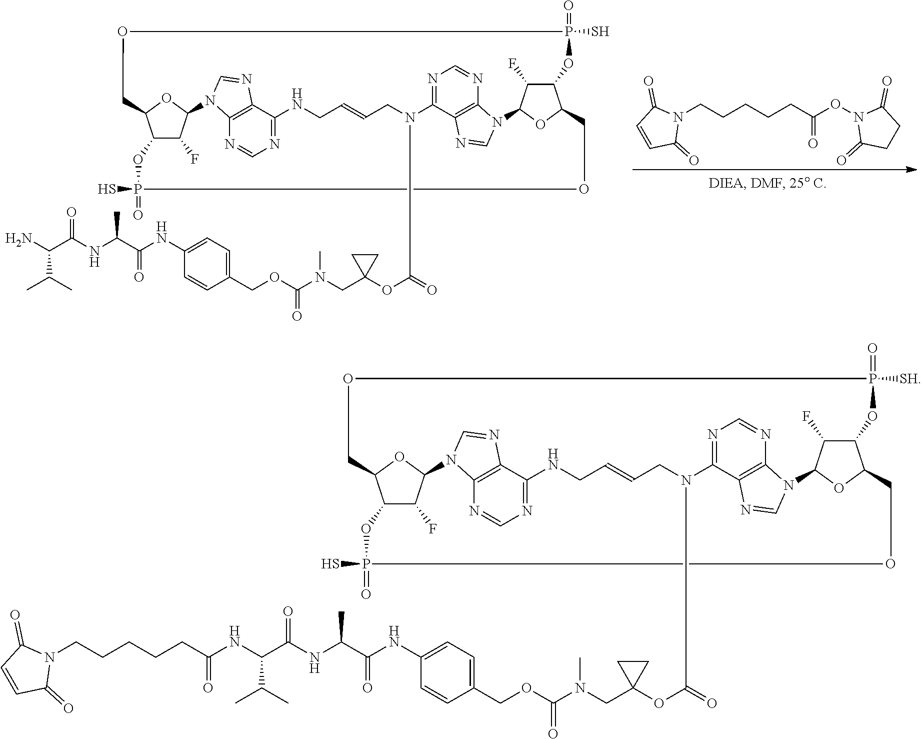 [SMILES: CC(C)[C@H](N)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

### Synthesis of 1-hydroxy-N-[(4-methoxyphenyl)methyl]-N-methylcyclopropane-1-carboxamide

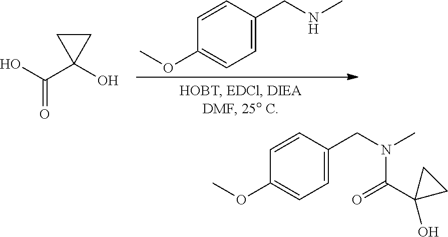

HOBT (1.99 g, 14.692 mmol) and EDCI (2.82 g, 14.692 mmol) were added to a stirred solution of 1-hydroxycyclopropane-1-carboxylic acid (1 g, 9.795 mmol) in DMF (10 mL). The resulting mixture was stirred at 25° C. for 30 min. Then [(4-methoxyphenyl)methyl](methyl)amine (1.48 g, 9.795 mmol) and DIEA (3.80 g, 29.385 mmol) were added to the resulting mixture. The resulting mixture was stirred at 25° C. for 2 h. The resulting mixture was diluted with EtOAc and washed with water. The organic layer was concentrated under reduced pressure. The residue was purified by silica gel column chromatography, eluted with PE/EtOAc (1/2) to afford 1.8 g of 1-hydroxy-N-[(4-methoxyphenyl)methyl]-N-methylcyclopropane-1-carboxamide as a white solid. LC-MS (ESI): 236.1 [M+H]⁺.

### Synthesis of 1-({[(4-methoxyphenyl)methyl](methyl)amino}methyl)cyclopropan-1-ol

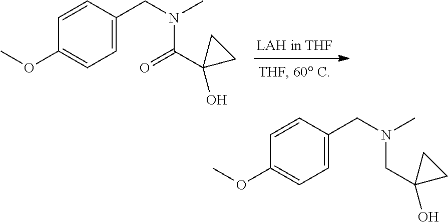 [SMILES: COc1ccc(CN(C)C(=O)C2(O)CC2)cc1 ; COc1ccc(CN(C)CC2(O)CC2)cc1]

LiAIH₄ in THF (7.65 mL, 7.650 mmol) was added to a stirred solution of 1-hydroxy-N-[(4-methoxyphenyl)methyl]-N-methylcyclopropane-1-carboxamide (1.8 g, 7.650 mmol) in THF (10 mL). The resulting mixture was stirred at 60° C. for 2 h. The resulting mixture was concentrated under reduced pressure. The residue was purified by silica gel column chromatography, eluted with CH₂Cl₂/MeOH (10/1) to afford 780 mg of 1-({[(4-methoxyphenyl)methyl](methyl)amino}methyl)cyclopropan-1-ol as a light-yellow oil. LC-MS (ESI): 222.1 [M+H]⁺.

### Synthesis of 1-[(methylamino)methyl]cyclopropan-1-ol

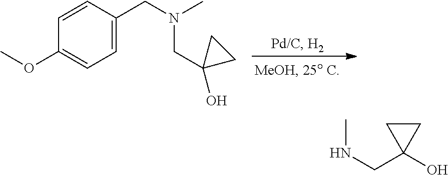

Pd/C (220 mg) was added to a stirred solution of 1-({[(4-methoxyphenyl)methyl](methyl)amino}methyl)cyclopropan-1-ol (1.1 g, 4.971 mmol) in MeOH (10 mL). The resulting mixture was stirred at 25° C. for 2 h under the atmosphere of hydrogen. The resulting mixture was filtered. The filtrate was concentrated under reduced pressure. The residue was purified by SCX to obtain 214 mg of 1-[(methylamino)methyl]cyclopropan-1-ol as a light-brown oil. LC-MS (ESI): 102.0 [M+H]⁺.

### Synthesis of {4-[(2S)-2-[(2S)-2-{[(tert-butoxy)carbonyl]amino}-3-methylbutanamido]propanamido]phenyl}methyl N-[(1-hydroxycyclopropyl)methyl]-N-methylcarbamate

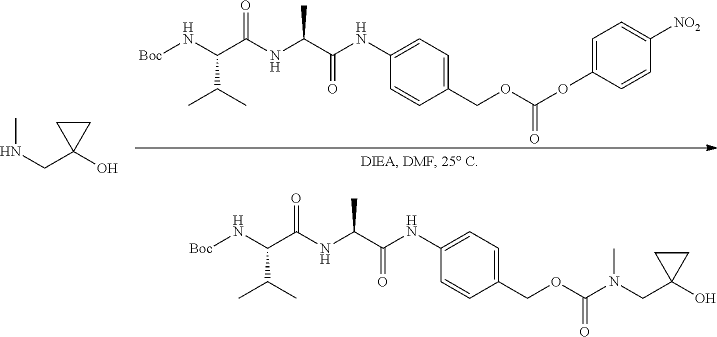

{4-[(2S)-2-[(2S)-2-[(tert-butoxycarbonyl)amino]-3-methylbutanamido]propanamido]phenyl}methyl 4-nitrophenyl carbonate (1.10 g, 1.977 mmol) and DIEA (511.12 mg, 3.954 mmol) were added to a stirred solution of 1-[(methylamino)methyl]cyclopropan-1-ol (200 mg, 1.977 mmol) in DMF (10 mL). The resulting mixture was stirred at 25° C. for 14 h. The resulting mixture was concentrated under reduced pressure. The residue was purified by Prep-TLC (DCM/MeOH 10/1) to afford 504 mg of {4-[(2S)-2-[(2S)-2-{[(tert-butoxy)carbonyl]amino}-3-methylbutanamido]propanamido]phenyl}methyl N-[(1-hydroxycyclopropyl)methyl]-N-methylcarbamate as a light-yellow oil. LC-MS (ESI): 521.2 [M+H]⁺.

### Synthesis of 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl 4-nitrophenyl carbonate

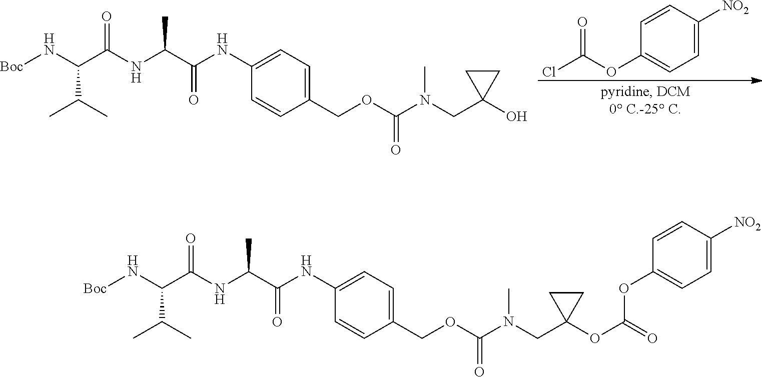 [SMILES: CC(C)[C@H](NC(=O)OC(C)(C)C)C(=O)N[C@@H](C)C(=O)Nc1ccc(COC(=O)N(C)CC2(O)CC2)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N(C)CC2(OC(=O)Oc3ccc([N+](=O)[O-])cc3)CC2)cc1]

{4-[(2S)-2-[(2S)-2-[(tert-butoxycarbonyl)amino]-3-methylbutanamido]propanamido]phenyl}methyl 4-nitrophenyl carbonochloridate (174.22 mg, 0.864 mmol) was added to a stirred solution of {4-[(2S)-2-[(2S)-2-{[(tert-butoxy)carbonyl]amino}-3-methylbutanamido]propanamido]phenyl}methyl N-[(1-hydroxycyclopropyl)methyl]-N-methylcarbamate (300 mg, 0.576 mmol) in DCM (10 mL). Then pyridine (68.37 mg, 0.864 mmol) was added to the resulting mixture at 0° C. The resulting mixture was stirred at 25° C. for 14 h. The resulting mixture was concentrated under reduced pressure. The residue was purified by silica gel column chromatography, eluted with PE/EtOAc (1/3) to afford 303 mg of 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl 4-nitrophenyl carbonate as a white solid. LC-MS (ESI): 686.3 [M+H]⁺.

### Synthesis of 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

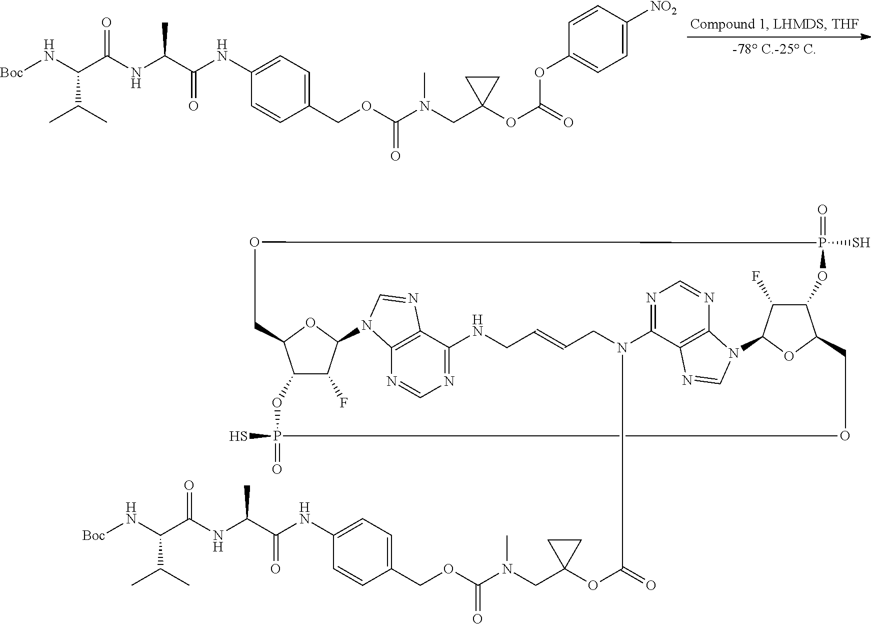 [SMILES: CC(C)[C@H](NC(=O)OC(C)(C)C)C(=O)N[C@@H](C)C(=O)Nc1ccc(COC(=O)N(C)CC2(OC(=O)Oc3ccc([N+](=O)[O-])cc3)CC2)cc1 ; CC(C)[C@H](NC(=O)OC(C)(C)C)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

LiHMDS (0.60 mL, 0.600 mmol) was added to a stirred solution of Compound 1 (75 mg, 0.100 mmol) in THF (3 mL) at −78° C. The resulting mixture was stirred at −78° C. for 30 min under the atmosphere of nitrogen. Then 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl 4-nitrophenyl carbonate (68.89 mg, 0.100 mmol) was added to the resulting mixture. The resulting mixture was slowly warmed to 25° C. over 1 hour under the atmosphere of nitrogen. The reaction was quenched with AcOH. The resulting mixture was concentrated under reduced pressure. The residue was purified by Prep-TLC (EtOAc(1% TEA)/MeOH 3/1) to afford 55 mg of 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a white solid. LC-MS (ESI): 1293.3 [M+H]⁺.

### Synthesis of 1-({[({4-[(2S)-2-[(2S)-2-amino-3-methylbutanamido]propanamido]phenyl}methoxy)carbonyl](methyl)amino}methyl)cyclopropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate F

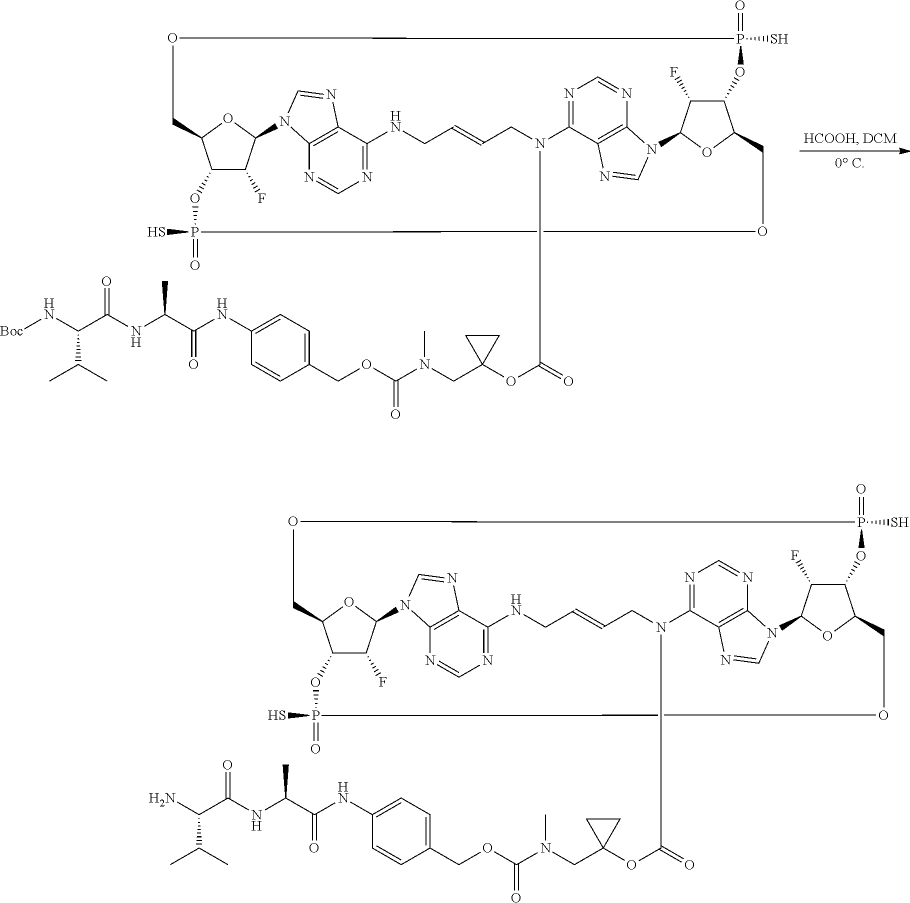 [SMILES: CC(C)[C@H](NC(=O)OC(C)(C)C)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; CC(C)[C@H](N)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

HCOOH (2 mL) was added to a stirred solution of 1-({[({4-[(2S)-2-[(2S)-2-{[(tert-butoxy)carbonyl]amino}-3-methylbutanamido]propanamido]phenyl}methoxy)carbonyl](methyl)amino}methyl)cyclopropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (55 mg, 0.042 mmol) in DCM (2 mL) at 0° C. The resulting mixture was stirred at 0° C. for 1 h. The resulting mixture was concentrated under reduced pressure to obtain the crude product of 1-({[({4-[(2S)-2-[(2S)-2-amino-3-methylbutanamido]propanamido]phenyl}methoxy)carbonyl](methyl)amino}methyl)cyclopropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate) as a colorless oil. LC-MS (ESI): 1193.3 [M+H]⁺.

### Synthesis of 1-({[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}methyl)cyclopropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP8)

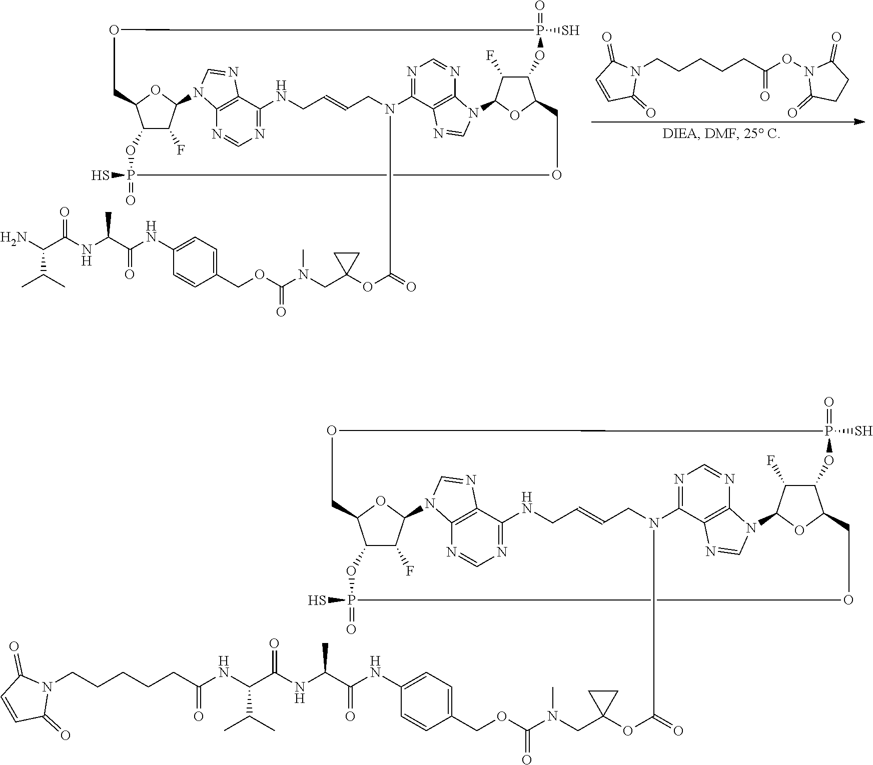 [SMILES: CC(C)[C@H](N)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)CC2(OC(=O)N3C/C=C/CNc4ncnc5c4ncn5[C@@H]4O[C@@H]5CO[P@](=O)(S)O[C@H]6[C@@H](F)[C@@H](O[C@@H]6CO[P@@](=O)(S)O[C@H]5[C@H]4F)n4cnc5c3ncnc54)CC2)cc1]

2,5-dioxopyrrolidin-1-yl 6-(2,5-dioxopyrrol-1-yl)hexanoate (12.92 mg, 0.042 mmol) and DIEA (16.25 mg, 0.126 mmol) were added to a stirred solution of 1-({[({4-[(2S)-2-[(2S)-2-amino-3-methylbutanamido]propanamido]phenyl}methoxy)carbonyl](methyl)amino}methyl)cyclopropyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (50 mg, 0.042 mmol) in DMF (2 mL). The resulting mixture was stirred at 25° C. for 14 h. The resulting mixture was concentrated under reduced pressure. The residue was purified by Prep-TLC (EtOAc(1% TEA)/MeOH 3/1) and Prep-HPLC with the following conditions (Column: XBridge Prep OBD C18 Column, 19×250 mm, 5 μm; Mobile Phase A: Water (50 mmol NH₄HCO₂), Mobile Phase B: MECN; Flow rate: 25 mL/min; Gradient: 25% B to 35% B in 10 min, 35% B; Wavelength: 220/254 nm) to afford 12.5 mg of 1-({[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}methyl)cyclopropyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP8) as a white solid.

LC-MS (ESI): 1386.40 [M+H]⁺.

¹H NMR (400 MHz, Methanol-d₄) δ 9.03-8.80 (m, 1H), 8.80-8.57 (m, 2H), 8.39-8.20 (m, 1H), 8.18-8.02 (m, 1H), 7.68-7.50 (m, 2H), 7.34-7.21 (m, 2H), 6.82-6.73 (m, 2H), 6.59-6.24 (m, 2H), 6.14-5.86 (m, 1H), 5.85-5.28 (m, 3H), 5.12-4.98 (m, 2H), 4.84-4.72 (m, 2H), 4.71-4.55 (m, 3H), 4.53-4.35 (m, 5H), 4.23-4.10 (m, 1H), 4.12-4.02 (m, 1H), 3.99-3.88 (m, 2H), 3.77-3.55 (m, 1H), 3.54-3.44 (m, 3H), 3.44-3.37 (m, 1H), 2.96-2.79 (m, 3H), 2.36-2.22 (m, 2H), 2.18-2.00 (m, 1H), 1.73-1.51 (m, 4H), 1.51-1.41 (m, 3H), 1.37-1.24 (m, 2H), 1.23-1.10 (m, 1H), 1.05-0.94 (m, 6H), 0.94-0.60 (m, 3H).

### 9. Synthesis of LP9

The synthesis of LP9 is shown below:

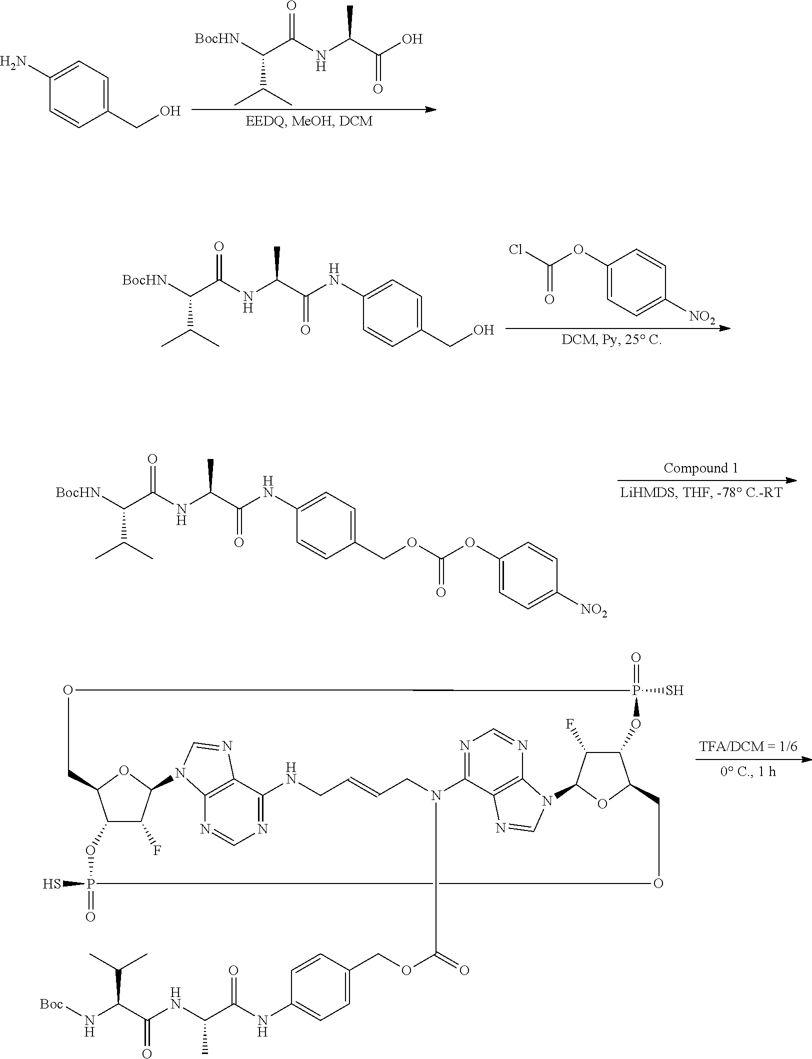 [SMILES: Nc1ccc(CO)cc1 ; CC(C)[C@H](NC(=O)OC(C)(C)C)C(=O)N[C@@H](C)C(=O)O ; CC(C)[C@H](NC(=O)OC(C)(C)C)C(=O)N[C@@H](C)C(=O)Nc1ccc(CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

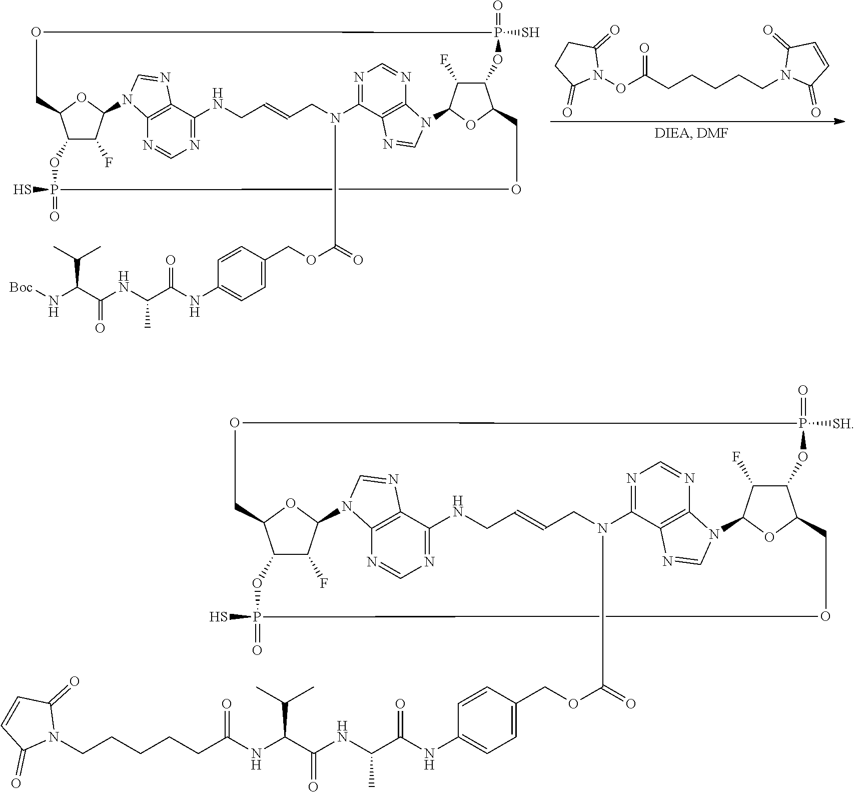

### Synthesis of tert-butyl N-[(1S)-1-{[(1S)-1-{[4-(hydroxymethyl)phenyl]carbamoyl}ethyl]carbamoyl}-2-methylpropyl]carbamate

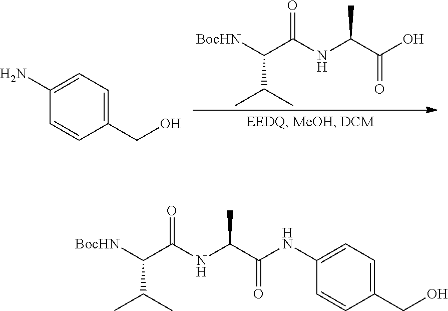 [SMILES: Nc1ccc(CO)cc1 ; CC(C)[C@H](NC(=O)OC(C)(C)C)C(=O)N[C@@H](C)C(=O)O ; CC(C)[C@H](NC(=O)OC(C)(C)C)C(=O)N[C@@H](C)C(=O)Nc1ccc(CO)cc1]

Ethyl 2-ethoxy-1,2-dihydroquinoline-1-carboxylate (32.13 g, 129.918 mmol) and (2S)-2-[(2S)-2-[(tert-butoxycarbonyl)amino]-3-methylbutanamido]propanoic acid (18.73 g, 64.959 mmol) were added to a stirred solution of p-aminobenzylalcohol (8 g, 64.959 mmol) in MeOH (5 mL)/DCM (30 mL). The resulting mixture was stirred at 25° C. for 2 h. The resulting mixture was concentrated under reduced pressure. The residue was purified by silica gel column chromatography, eluted with CH₂Cl₂/MeOH (10/1) to afford 23 g of tert-butyl N-[(1S)-1-{[(1S)-1-{[4-(hydroxymethyl)phenyl]carbamoyl}ethyl]carbamoyl}-2-methylpropyl]carbamate as a light-yellow solid. LC-MS (ESI): 394.2 [M+H]⁺.

### Synthesis of {4-[(2S)-2-[(2S)-2-{[(tert-butoxy)carbonyl]amino}-3-methylbutanamido]propanamido]phenyl}methyl 4-nitrophenyl carbonate

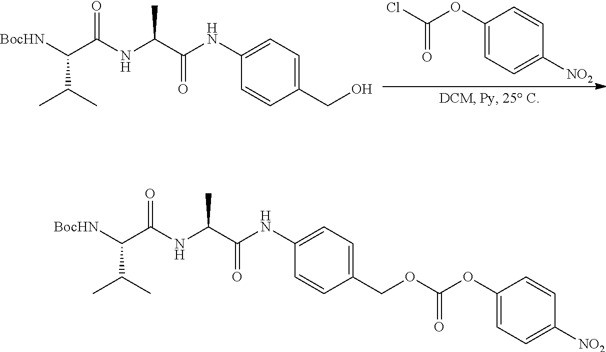 [SMILES: CC(C)[C@H](NC(=O)OC(C)(C)C)C(=O)N[C@@H](C)C(=O)Nc1ccc(CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)Oc2ccc([N+](=O)[O-])cc2)cc1]

4-nitrophenyl carbonochloridate (768.37 mg, 3.811 mmol) and pyridine (301.54 mg, 3.811 mmol) were added to a stirred solution of tert-butyl N-[(1S)-1-{[(1S)-1-{[4-(hydroxymethyl)phenyl]carbamoyl}ethyl]carbamoyl}-2-methylpropyl]carbamate (1 g, 2.541 mmol) in DCM (20 mL). The resulting mixture was stirred at 25° C. for 2 h. The resulting mixture was concentrated under reduced pressure. The residue was purified by silica gel column chromatography, eluted with PE/EtOAc (1/1) to afford 1.4 g of {4-[(2S)-2-[(2S)-2-{[(tert-butoxy)carbonyl]amino}-3-methylbutanamido]propanamido]phenyl}methyl 4-nitrophenyl carbonate as a white solid. LC-MS (ESI): 559.2 [M+H]⁺.

### Synthesis of {4-[(2S)-2-[(2S)-2-{[(tert-butoxy)carbonyl]amino}-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylatedeia

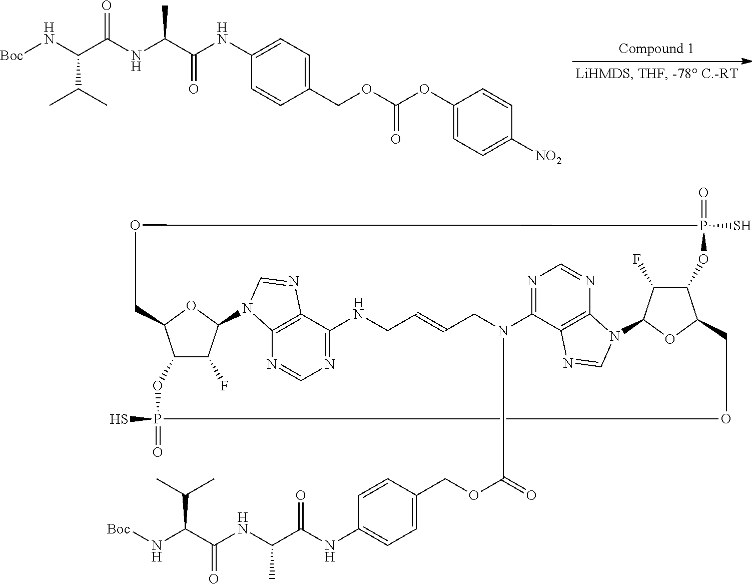 [SMILES: CC(C)[C@H](NC(=O)OC(C)(C)C)C(=O)N[C@@H](C)C(=O)Nc1ccc(COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

LiHMDS (0.40 mL, 0.402 mmol) was added to a stirred solution of Compound 1 (50 mg, 0.067 mmol) in THF (2 mL). The resulting mixture was stirred at −78° C. for 30 min. Then {4-[(2S)-2-[(2S)-2-{[(tert-butoxy)carbonyl]amino}-3-methylbutanamido]propanamido]phenyl}methyl 4-nitrophenyl carbonate (37.41 mg, 0.067 mmol) was added to the resulting mixture. The resulting mixture was stirred at −78° C. to 25° C. over 2 h under the atmosphere of nitrogen. The reaction was quenched with AcOH. The resulting mixture was concentrated under reduced pressure. The residue was purified by Prep-TLC (EtOAc(1% TEA)/MeOH 3/1) to afford 45 mg of {4-[(2S)-2-[(2S)-2-{[(tert-butoxy)carbonyl]amino}-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a white solid LC-MS (ESI): 1166.2 [M+H]⁺.

### Synthesis of {4-[(2S)-2-[(2S)-2-amino-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

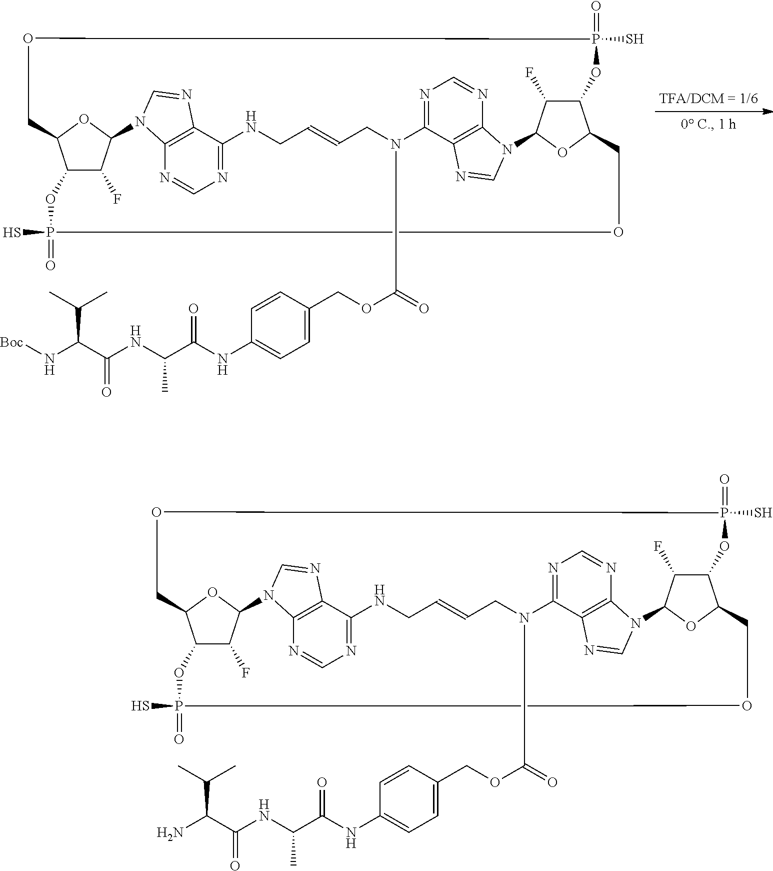 [SMILES: CC(C)[C@H](NC(=O)OC(C)(C)C)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

TFA (1 mL) was added to a stirred solution of {4-[(2S)-2-[(2S)-2-[(tert-butoxycarbonyl)amino]-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (45 mg, 0.039 mmol) in DCM (6 mL). The resulting mixture was stirred at 0° C. for 1 h. The resulting mixture was concentrated under reduced pressure to obtain the crude product of {4-[(2S)-2-[(2S)-2-amino-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a colorless oil. LC-MS (ESI): 1066.2 [M+H]⁺.

### Synthesis of {4-[(2S)-2-[(2S)-2-[6-(2,5-dioxo-2,5-dihydro-1H-pyrrol-1-yl)hexanamido]-3-methylbutanamido]propanamido]phenyl}methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP9)

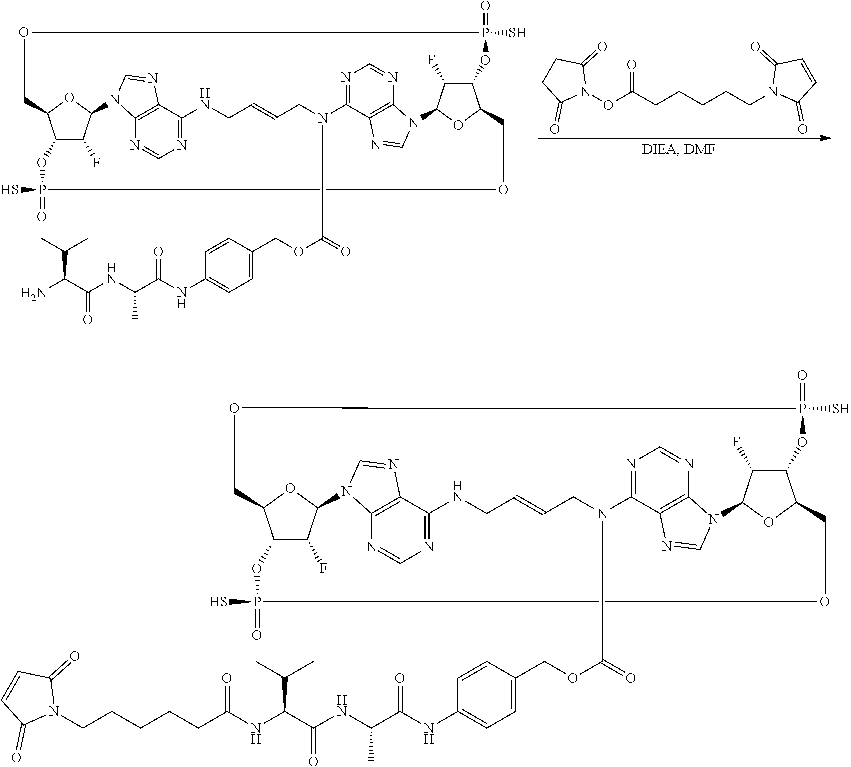 [SMILES: CC(C)[C@H](N)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

DIEA (24.86 mg, 0.190 mmol) and 2,5-dioxopyrrolidin-1-yl 6-(2,5-dioxopyrrol-1-yl)hexanoate (11.86 mg, 0.038 mmol) were added to a stirred solution of {4-[(2S)-2-[(2S)-2-amino-3-methylbutanamido]propanamido]phenyl}methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (41 mg, 0.038 mmol) in DMF (2 mL). The resulting mixture was stirred at 25° C. for 14 h. The resulting mixture was concentrated under reduced pressure. The residue was purified by Prep-TLC (CH₂Cl₂(1% TEA)/MeOH 8/1) and then Prep-TLC (EtOAc(1% TEA)/MeOH 3/1). The crude product was purified by Prep-HPLC with the following conditions (Column: XBridge Prep Phenyl OBD Column, 19×250 mm, 5 μm; Mobile Phase A: Water(50 mmol HCO₂NH₄), Mobile Phase B: MeCN; Flow rate: 25 mL/min; Gradient: 20% B to 40% B in 15 min, 40% B; Wavelength: 254 nm to afford 5.1 mg of {4-[(2S)-2-[(2S)-2-[6-(2,5-dioxo-2,5-dihydro-1H-pyrrol-1-yl)hexanamido]-3-methylbutanamido]propanamido]phenyl}methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP9) as a white solid.

LC-MS (ESI): 1259.45 [M+H]⁺.

¹H NMR (400 MHz, Methanol-d₄) δ 8.92 (s, 1H), 8.77-8.69 (m, 2H), 8.31 (s, 1H), 8.15 (s, 1H), 7.53 (d, J=8.25 Hz, 2H), 7.23 (d, J=8.00 Hz, 2H), 6.85-6.67 (m, 2H), 6.52-6.41 (m, 1H), 6.39-6.25 (m, 1H), 6.09-5.40 (m, 4H), 5.30-5.15 (m, 2H), 5.09-4.95 (m, 2H), 4.66-4.56 (m, 3H), 4.55-4.45 (m, 2H), 4.44-4.34 (m, 2H), 4.22-4.13 (m, 1H), 4.12-4.01 (m, 1H), 4.00-3.92 (m, 1H), 3.72-3.55 (m, 1H), 3.52-3.43 (m, 2H), 2.36-2.18 (m, 2H), 2.15-2.04 (m, 1H), 1.72-1.53 (m, 5H), 1.48-1.40 (m, 3H), 1.36-1.25 (m, 4H), 1.04-0.90 (m, 6H).

### 10. Synthesis of LP10

The synthesis of LP10 is shown below:

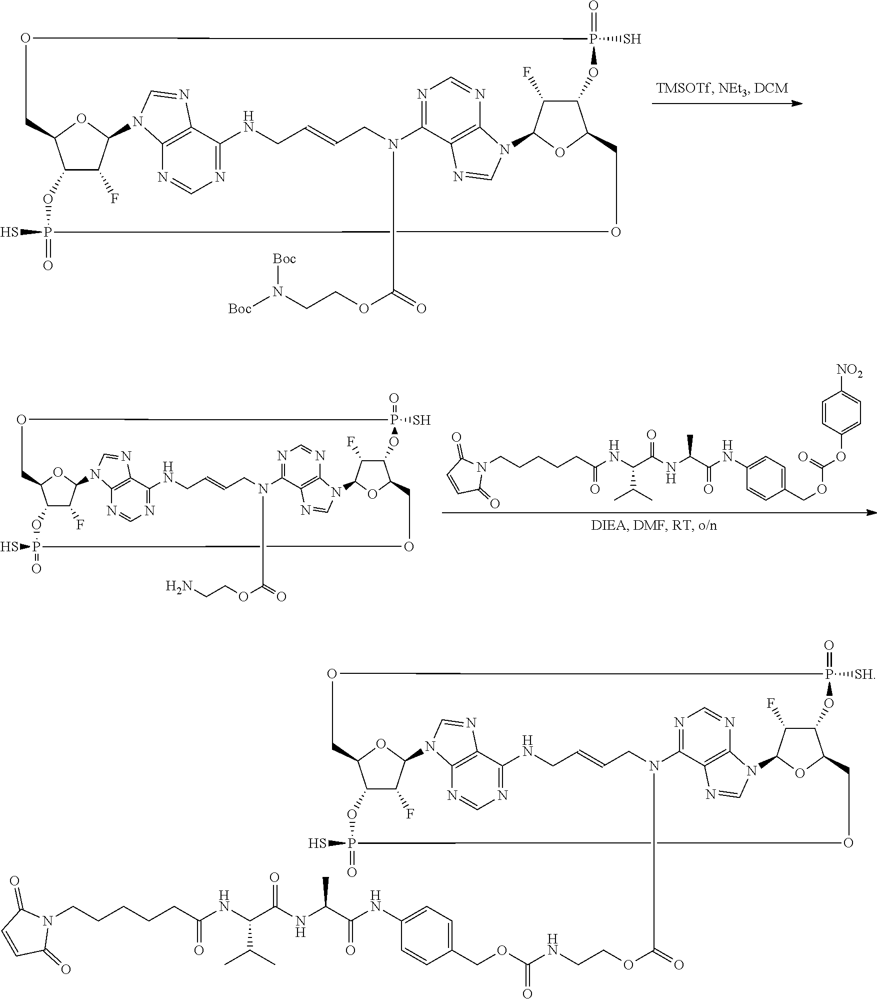

### Synthesis of 2-aminoethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

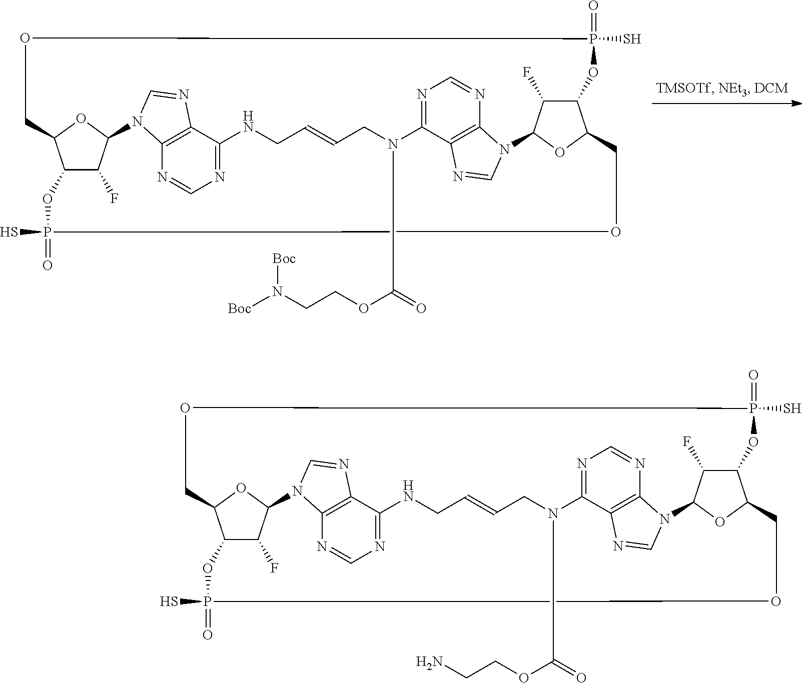 [SMILES: CC(C)(C)OC(=O)N(CCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32)C(=O)OC(C)(C)C ; NCCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32]

To a mixture of 2-{bis[(tert-butoxy)carbonyl]amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg, 0.039 mmol) in DCM (2 mL) was added TEA (19.57 mg, 0.195 mmol) and TMSOTf (60.19 mg, 0.273 mmol) at room temperature. The resulting mixture was stirred at room temperature for 1 h. The resulting mixture was concentrated under reduced pressure. The crude product was used in the next step directly without further purification. LC-MS (ESI): 834.2 [M+H]⁺.

### Synthesis of 2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP10)

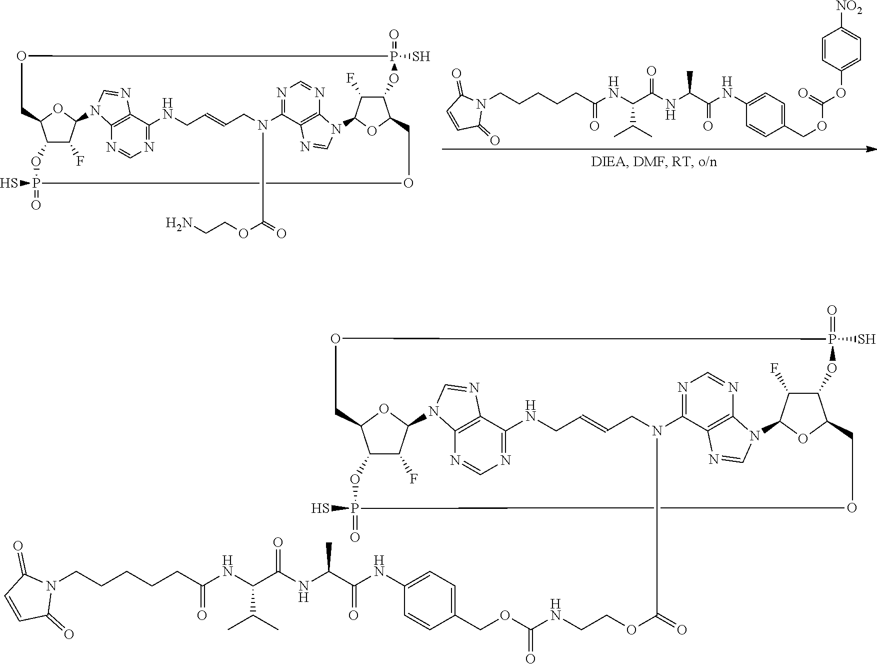 [SMILES: NCCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](C)C(=O)Nc1ccc(COC(=O)NCCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of 2-aminoethyl (1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (32 mg, 0.038 mmol) and {4-[(25)-2-[(25)-2-[6-(2,5-dioxopyrrol-1-yl)hexanamido]-3-methylbutanamido]propanamido]phenyl}methyl 4-nitrophenyl carbonate (25.01 mg, 0.038 mmol) in DMF(2 mL) was added DIEA (24.81 mg, 0.190 mmol) at room temperature. The resulting mixture was stirred at room temperature overnight. The resulting mixture was concentrated under vacuum. The residue was purified by Prep-TLC(EtOAc/MeOH 3:1) and Prep-HPLC with the following conditions (Column: XBridge Prep Phenyl OBD Column, 19*250 mm, 5 μm; Mobile Phase A: Water (50 mmol/L HCO₂NH₄), Mobile Phase B: MeCN; Flow rate: 25 mL/min; Gradient: 25% B to 50% B in 10 min, 50% B; Wavelength: 254 nm to afford 2-{[({4-[(25)-2-[(25)-2-[6-(2,5-dioxo-2,5-dihydro-1H-pyrrol-1-yl)hexanamido]-3-methylbutanamido]propanamido]phenyl}methoxy)carbonyl]amino}ethyl(1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP10) (7.6 mg) as a white solid.

LC-MS (ESI): 1346.45 [M+H]⁺.

¹H NMR (400 MHz, Methanol-d₄) δ 8.92 (s, 2H), 8.73 (m, 1H), 8.19 (m, 2H), 7.57 (m, 2H), 7.30 (m, 2H), 6.79 (s, 2H), 6.39 (m, 1H), 6.31 (in, 1H),6.05 (s, 1H) 5.92 (s, 1H), 5.78 (s, 1H), 5.58-5.45 (in, 2H), 5.02 (m,2H), 4.99 (m, 3H), 4.61 (in, 5H), 4.55-4.36 (m, 2H), 4.36 (m, 2H), 4.21 (in, 1H),4.14 (m, 1H), 4.04 (in, 2H),3.76 (m, 1H), 3.52 (in, 1H),3.55-3.40 (m, 2H), 3.15 (m, 3H), 2.29 (m, 2H), 2.09 (m, 1H), 1.94 (m, 4H), 1.61 (m, 3H), 1.37-1.18 (m, 2H), 0.98 (m, 6H).

### 11. Synthesis of LP11

The synthesis of LP11 is shown below:

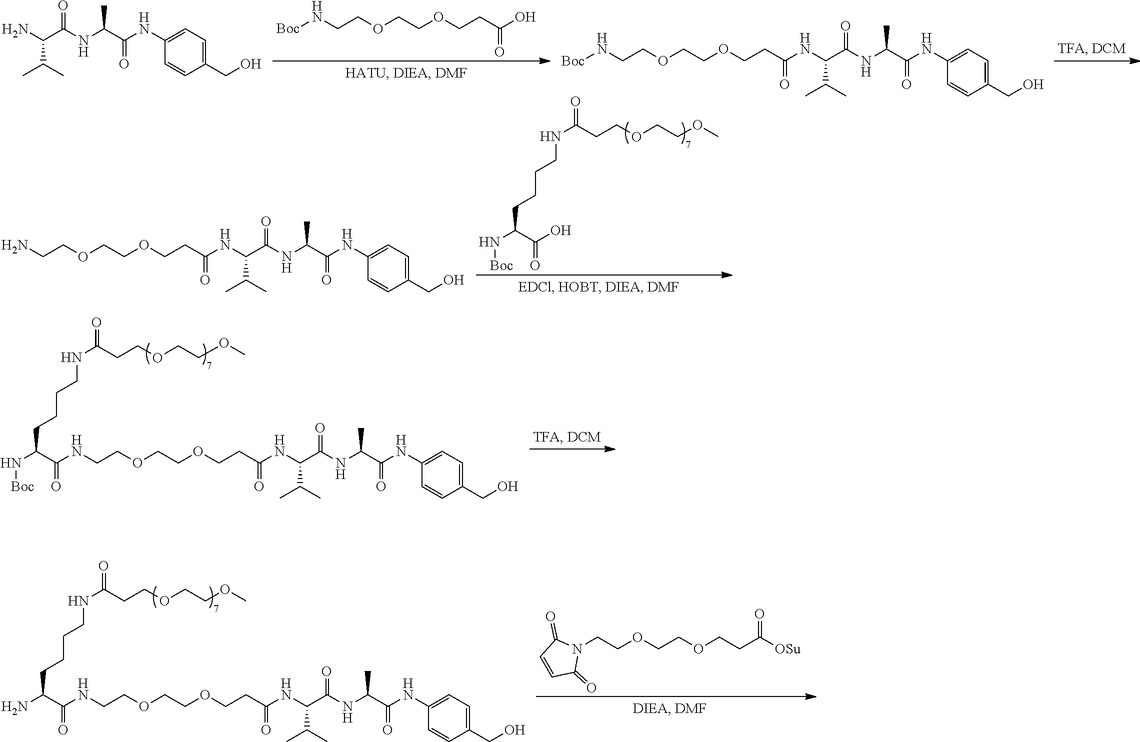

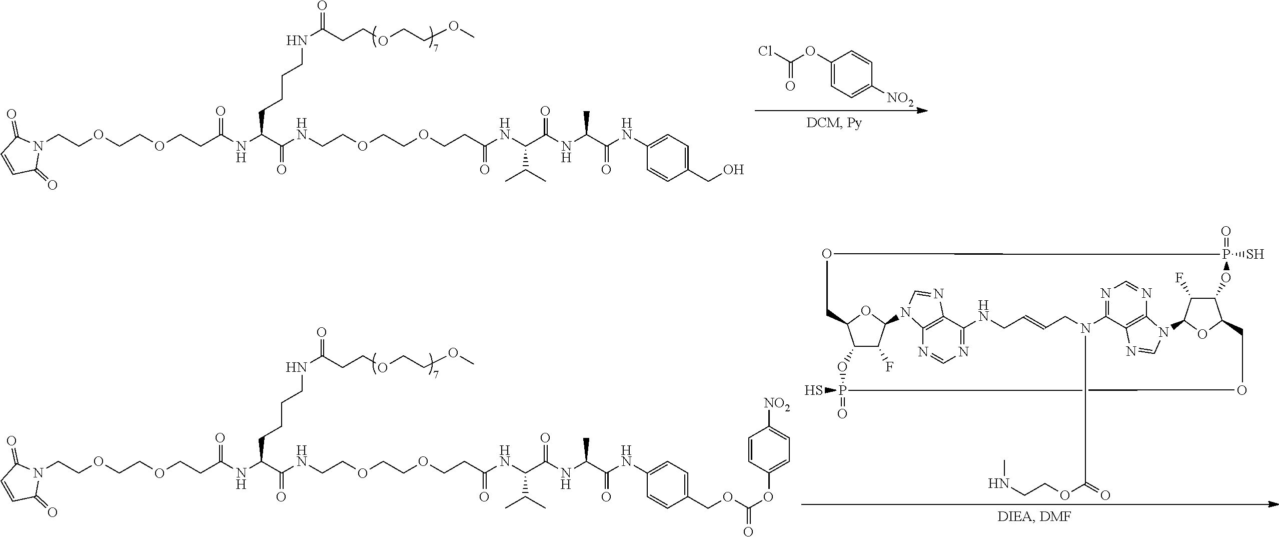

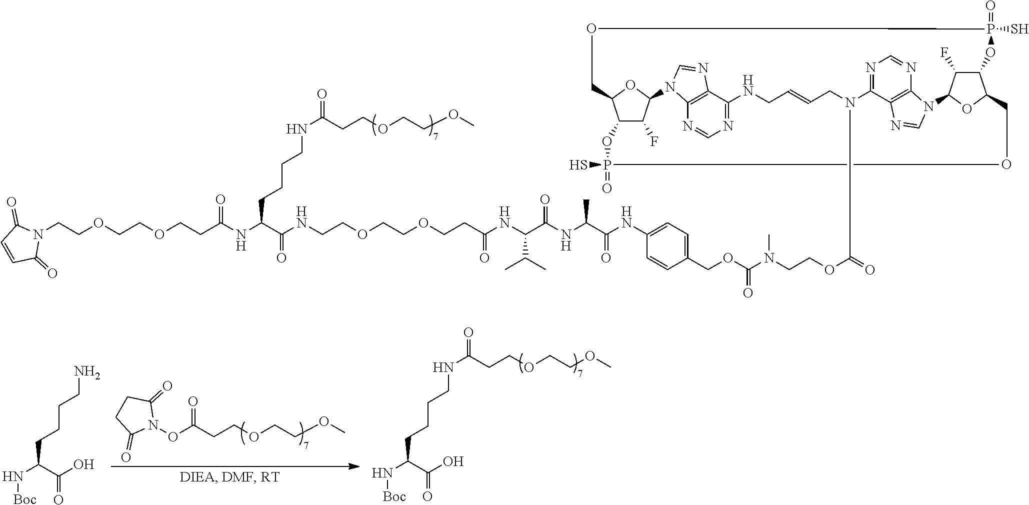

### Synthesis of tert-butyl N-{2-[2-(2-{{(1S)-1-([[(1S)-1-{[4-(hydroxymethyl)phenyl]carbamoyl}ethyl]carbamoyl}-2-methylpropyl]carbamoyl}ethoxy)ethoxy]ethyl}carbamate

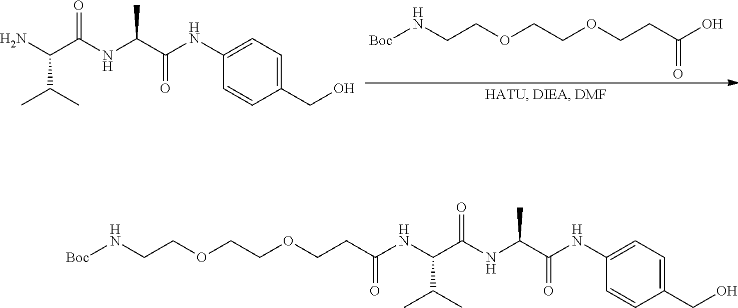 [SMILES: CC(C)[C@H](N)C(=O)N[C@@H](C)C(=O)Nc1ccc(CO)cc1 ; CC(C)(C)OC(=O)NCCOCCOCCC(=O)O ; CC(C)[C@H](NC(=O)CCOCCOCCNC(=O)OC(C)(C)C)C(=O)N[C@@H](C)C(=O)Nc1ccc(CO)cc1]

To the solution of (2S)-2-amino-N-[(1S)-1-{[4-(hydroxymethyl)phenyl]carbamoyl}ethyl]-3-methylbutanamide (400 mg, 1.363 mmol), 3-(2-{2-[(tert-butoxycarbonyl)amino]ethoxy}ethoxy)propanoic acid (378.12 mg, 1.363 mmol), HATU (570.29 mg, 1.499 mmol) in DMF (3 mL) was added DIEA (528.68 mg, 4.089 mmol). The solution was stirred at room temperature for 2 h. The resulting mixture was diluted with EtOAc and washed with H₂O. The resulting mixture was concentrated under reduced pressure. The residue was purified by Prep-TLC (CH2Cl2/MeOh 8:1) to afford tert-butyl N-{2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamate (420 mg) as a white solid. LC-MS (ESI): 555.3 [M+H]⁺.

### Synthesis of (2S)-2-{3-[2-(2-aminoethoxy)ethoxy]propanamido}-N-[(1S)-1-{[4-(hydroxymethyl)phenyl]carbamoyl}ethyl]-3-methylbutanamide

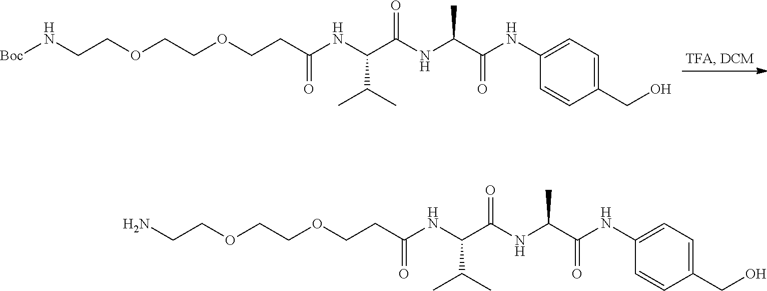 [SMILES: CC(C)[C@H](NC(=O)CCOCCOCCNC(=O)OC(C)(C)C)C(=O)N[C@@H](C)C(=O)Nc1ccc(CO)cc1 ; CC(C)[C@H](NC(=O)CCOCCOCCN)C(=O)N[C@@H](C)C(=O)Nc1ccc(CO)cc1]

The solution of tert-butyl N-{2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamate (420 mg, 0.760 mmol) in DCM (1.5 mL) was treated with TFA (1.5 mL) at room temperature. The resulting mixture was stirred at room temperature for 0.5 h. The mixture was concentrated under reduced pressure. The residue was dissolved in MeOH (2 mL)/H₂O (2 mL)/THF (2 mL). K₂CO₃ (577.66 mg, 4.180 mmol) was added to the above solution and stirred at room temperature for an hour. The resulting mixture was concentrated under reduced pressure. The residue was purified by Prep-TLC (CH₂Cl₂/MeOH 4:1) to afford (2S)-2-{3-[2-(2-aminoethoxy)ethoxy]propanamido}-N-[(1S)-1-{[4-(hydroxymethyl)phenyl]carbamoyl}ethyl]-3-methylbutanamide (300 mg) as a light-yellow solid. LC-MS (ESI): 453.3[M+H]⁺.

### Synthesis of (2S)-2-{[(tert-butoxy)carbonyl]amino}-6-(2,5,8,11,14,17,20,23-octaoxahexacosan-26-amido)hexanoic acid

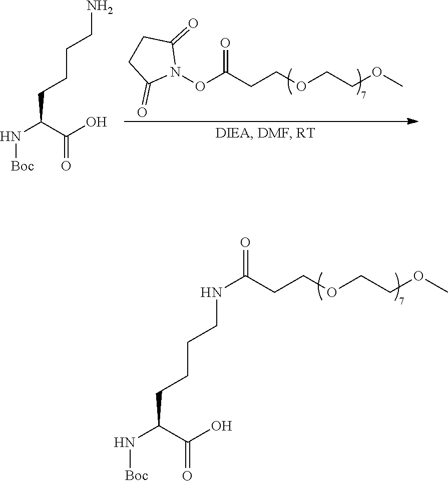

To a solution of (2S)-6-amino-2-{[(tert-butoxy)carbonyl]amino}hexanoic acid (287.61 mg, 1.168 mmol) and 2,5-dioxopyrrolidin-1-yl 2,5,8,11,14,17,20,23-octaoxahexacosan-26-oate (350 mg, 0.687 mmol) in DMF (3.00 mL) was added DIEA (266.33 mg, 2.061 mmol). The resulting mixture was stirred at room temperature for 2 h. The resulting mixture was concentrated under reduced pressure. The residue was purified by silica gel column chromatography, eluted with CH₂Cl₂/MeOH (5:1) to afford (2S)-2-{[(tert-butoxy)carbonyl]amino}-6-(2,5,8,11,14,17,20,23-octaoxahexacosan-26-amido)hexanoic acid (410 mg) as a colorless oil. LC-MS (ESI): 641.4[M+H]⁺.

### Synthesis of tert-butyl N-[(1S)-1-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)-5-(2,5,8,11,14,17,20,23-octaoxahexacosan-26-amido)pentyl]carbamate

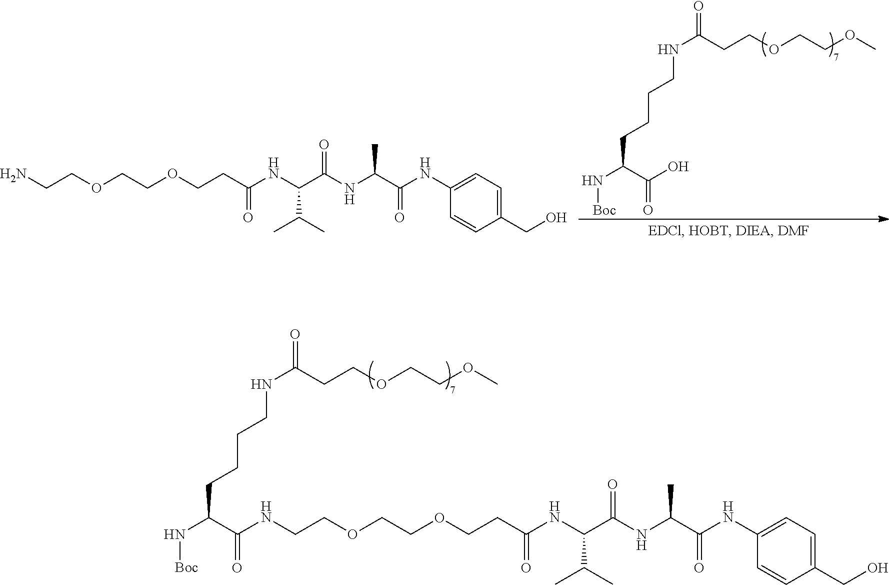

To a solution of (2S)-2-[(tert-butoxycarbonyl)amino]-6-(2,5,8,11,14,17,20,23-octaoxahexacosan-26-amido)hexanoic acid (290 mg, 0.453 mmol) in DMF (3 mL) was added HOBT (91.73 mg, 0.679 mmol), EDCI (130.14 mg, 0.679 mmol) at room temperature. The resulting mixture was stirred at room temperature for 0.5 h. To the above solution was added (2S)-2-{3-[2-(2-aminoethoxy)ethoxy]propanamido}-N-[(1S)-1-{[4-(hydroxymethyl)phenyl]carbamoyl}ethyl]-3-methylbutanamide (204.82 mg, 0.453 mmol) and DIEA (175.48 mg, 1.359 mmol) at room temperature. The resulting mixture was stirred at room temperature for an hour. The resulting mixture was concentrated under reduced pressure. The residue was purified by Prep-TLC (CH₂Cl₂/MeOH 5:1) to afford tert-butyl N-[(1S)-1-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)-5-(2,5,8,11,14,17,20,23-octaoxahexacosan-26-amido)pentyl]carbamate (437 mg) as a yellow oil. LC-MS (ESI): 1075.6[M+H]⁺.

### Synthesis of N-[(5S)-5-amino-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide

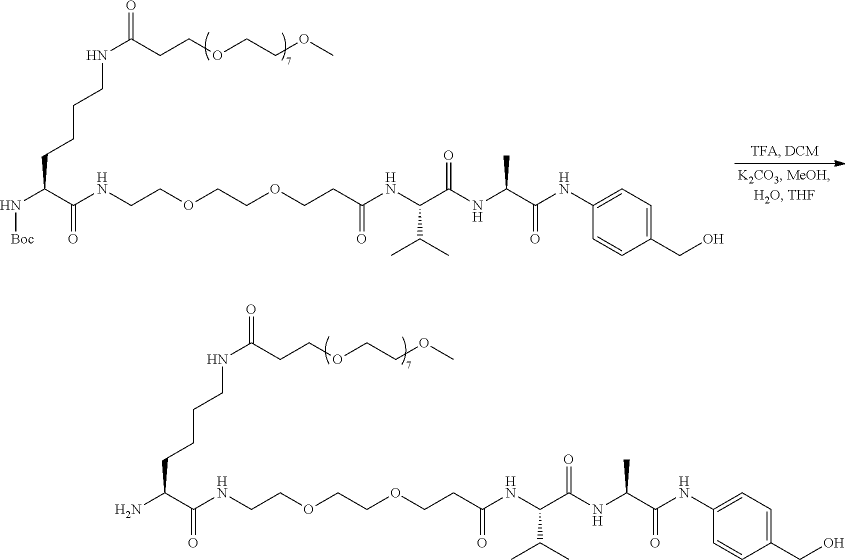

To a solution of tert-butyl N-[(1S)-1-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)-5-(2,5,8,11,14,17,20,23-octaoxahexacosan-26-amido)pentyl]carbamate (430 mg, 0.400 mmol) in DCM (2 mL) was added TFA (2 mL). The resulting mixture was stirred at room temperature for 0.5 h. The mixture was concentrated under reduced pressure. The residue was dissolved in MeOH (2 mL)/H₂O (2 mL)/THF (2 mL). K₂CO₃ (221.07 mg, 1.600 mmol) was added to the above solution and stirred at room temperature for an hour. The resulting mixture was concentrated under reduced pressure. The residue was purified by Prep-TLC (DCM/MeOH 5:1) to afford N-[(5S)-5-amino-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide (305 mg) as a yellow oil. LC-MS (ESI): 975.6 [M+H]⁺.

### Synthesis of N-[(5S)-5-(3-{2-[2-(2,5-dioxo-2,5-dihydro-1H-pyrrol-1-yl)ethoxy]ethoxy}propanamido)-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide

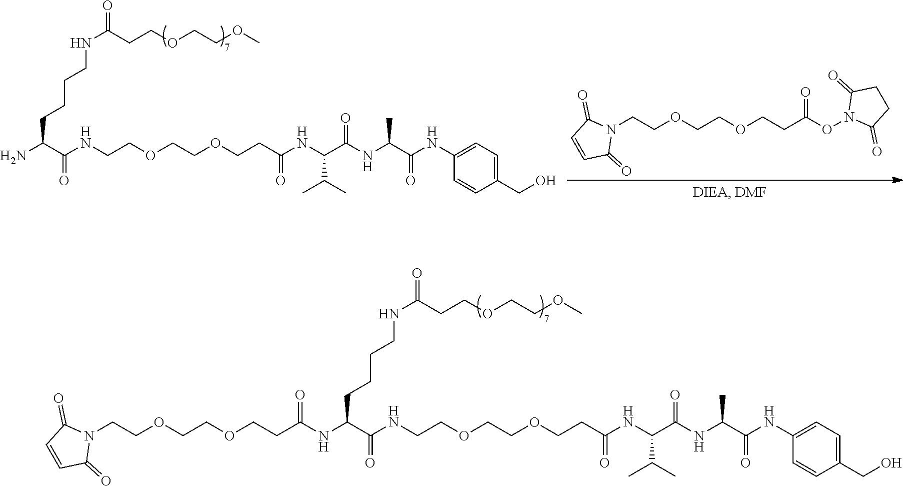

To a solution of N-[(5S)-5-amino-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide (305 mg, 0.313 mmol) and 2,5-dioxopyrrolidin-1-yl 3-{2-[2-(2,5-dioxopyrrol-1-yl)ethoxy]ethoxy}propanoate (132.98 mg, 0.376 mmol) in DMF (3.5 mL) was added DIEA (80.85 mg, 0.626 mmol). The resulting mixture was stirred at room temperature for 2 h. The resulting mixture was concentrated under reduced pressure. The residue was purified by Prep-TLC (DCM/MeOH 5:1) to afford N-[(5S)-5-(3-{2-[2-(2,5-dioxo-2,5-dihydro-1H-pyrrol-1-yl)ethoxy]ethoxy}propanamido)-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide (247 mg) as a light yellow solid. LC-MS (ESI): 1214.7[M+H]⁺.

### Synthesis of {4-[(2S)-2-[(2S)-2-[3-(2-{2-[(2S)-2-(3-{2-[2-(2,5-dioxo-2,5-dihydro-1H-pyrrol-1-yl)ethoxy]ethoxy}propanamido)-6-(2,5,8,11,14,17,20,23-octaoxahexacosan-26-amido)hexanamido]ethoxy}ethoxy)propanamido]-3-methylbutanamido]propanamido]phenyl}methyl 4-nitrophenyl carbonate

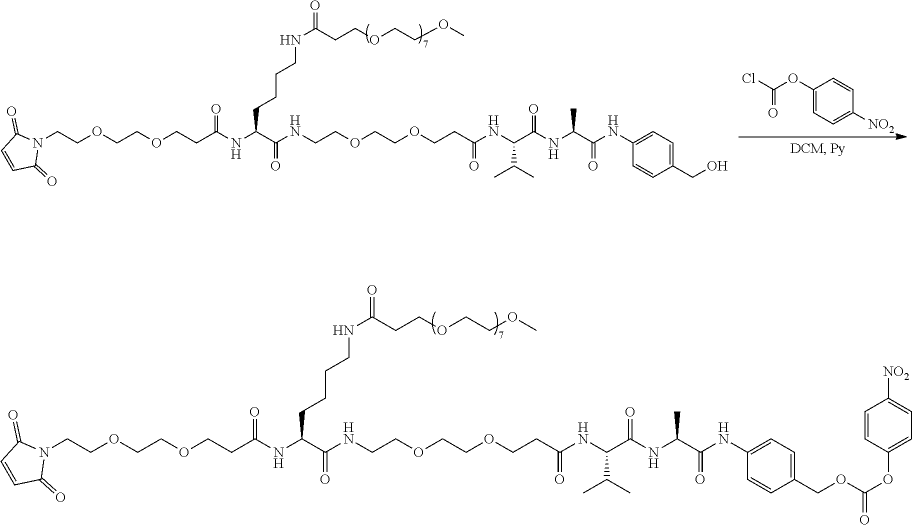

To a solution of N-[(5S)-5-(3-{2-[2-(2,5-dioxo-2,5-dihydro-1H-pyrrol-1-yl)ethoxy]ethoxy}propanamido)-5-({2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamoyl)pentyl]-2,5,8,11,14,17,20,23-octaoxahexacosan-26-amide (270 mg, 0.222 mmol) in DCM (3.5 mL) was added 4-nitrophenyl carbonochloridate (134.44 mg, 0.666 mmol) and Pyridine (52.76 mg, 0.666 mmol). The resulting mixture was stirred at room temperature for 2 h. The resulting mixture was concentrated under reduced pressure. The residue was purified by Prep-TLC (DCM/MeOH 8:1) to afford {4-[(2S)-2-[(2S)-2-[3-(2-{2-[(2S)-2-(3-{2-[2-(2,5-dioxo-2,5-dihydro-1H-pyrrol-1-yl)ethoxy]ethoxy}propanamido)-6-(2,5,8,11,14,17,20,23-octaoxahexacosan-26-amido)hexanamido]ethoxy}ethoxy)propanamido]-3-methylbutanamido]propanamido]phenyl}methyl 4-nitrophenyl carbonate (160 mg) as a light yellow oil. LC-MS (ESI): 1379.7[M+H]⁺.

### Synthesis of 2-{[({4-[(2S)-2-[(2S)-2-[3-(2-{2-[(2S)-2-(3-{2-[2-(2,5-dioxo-2,5-dihydro-1H-pyrrol-1-yl)ethoxy]ethoxy}propanamido)-6-(2,5,8,11,14,17,20,23-octaoxahexacosan-26-amido)hexanamido]ethoxy}ethoxy)propanamido]-3-methylbutanamido]propanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP11)

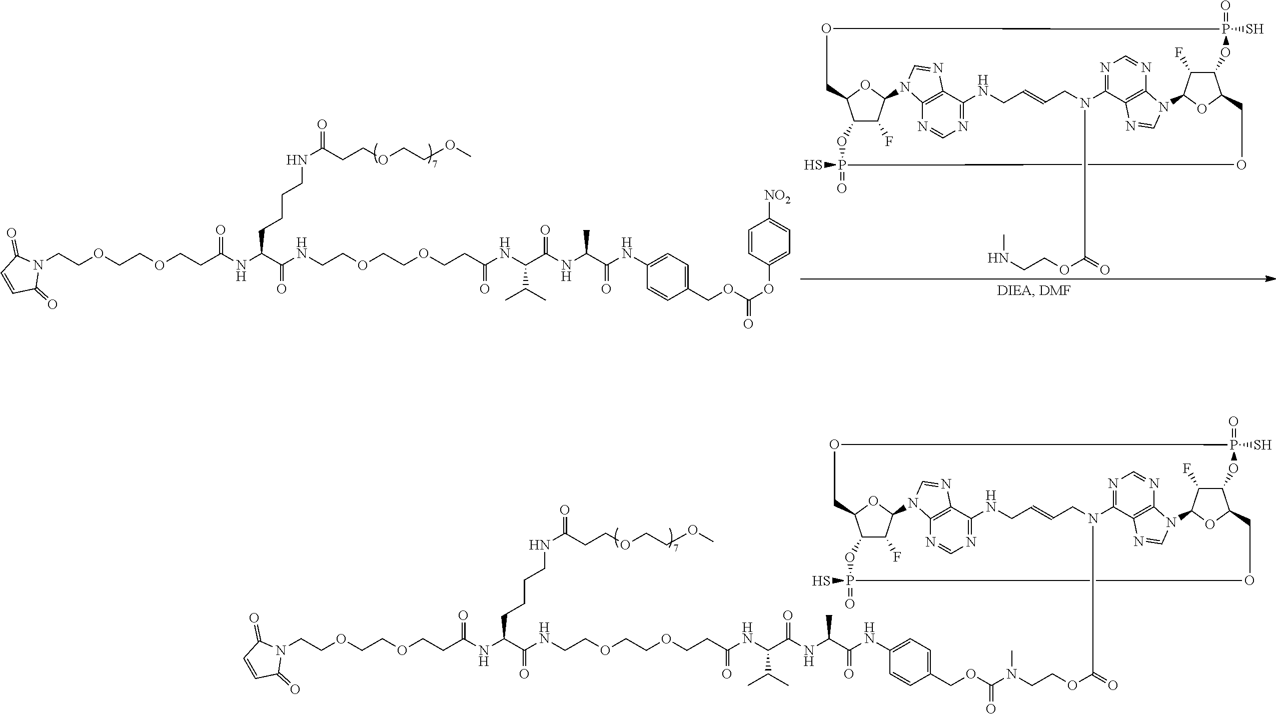

To a solution of {4-[(25)-2-[(25)-2-[3-(2-{2-[(25)-2-(3-{2-[2-(2,5-dioxo-2,5-dihydro-1H-pyrrol-1-yl)ethoxy]ethoxy}propanamido)-6-(2,5,8,11,14,17,20,23-octaoxahexacosan-26-amido)hexanamido]ethoxy}ethoxy)propanamido]-3-methylbutanamido]propanamido]phenyl}methyl 4-nitrophenyl carbonate (70 mg, 0.051 mmol) and 2-(methylamino)ethyl (1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (43.01 mg, 0.051 mmol) in DMF (3 mL) was added DIEA (45.91 mg, 0.357 mmol). The resulting mixture was stirred at room temperature for 14 h. The resulting mixture was concentrated under reduced pressure. The residue was purified by Prep-TLC (DCM(1% TEA)/MeOH 6:1) and Prep-H PLC(Column: XBridge Prep OBD C18 Column, 19×250 mm, 5 μm; Mobile Phase A: Water(50 mmol HCO₂NH₄), Mobile Phase B: MeCN; Flow rate: 25 mL/min; Gradient: 25% B to 35% B in 10 min, 35% B; Wavelength: 254 nm) to afford 2-{[({4-[(2S)-2-[(2S)-2-[3-(2-{2-[(2S)-2-(3-{2-[2-(2,5-dioxo-2,5-dihydro-1H-pyrrol-1-yl)ethoxy]ethoxy}propanamido)-6-(2,5,8,11,14,17,20,23-octaoxahexacosan-26-amido)hexanamido]ethoxy}ethoxy)propanamido]-3-methylbutanamido]propanamido]phenyl}methoxy)carbonyl](methyl)amino}ethyl(1R,3R,15Z,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP11) (6 mg) as a white solid.

LC-MS (ESI): 2087.77[M+H]⁺.

¹H NMR (400 MHz, Methanol-d₄) δ 8.91 (s, 1H), 8.84-8.56 (m, 2H), 8.13 (s, 1H), 7.66-7.55 (m, 2H), 7.32 (d, J=8.2 Hz, 1H), 7.21 (d, J=8.1 Hz, 1H), 6.83 (s, 2H), 6.57-6.25 (m, 2H), 6.09-5.88 (m, 1H), 5.84-5.04 (m, 5H), 4.73-4.48 (m, 6H), 4.47-4.32 (m, 4H), 4.30-4.13 (m, 1H), 4.10-3.91 (m, 2H), 3.84-3.69 (m, 7H), 3.69-3.47 (m, 50H), 3.26-3.14 (m, 3H), 2.85-2.69 (m, 3H), 2.63-2.41 (m, 7H), 2.15 (s, 2H), 1.90-1.76 (m, 1H), 1.73-1.60 (m, 1H), 1.56-1.26 (m, 10H), 1.09-0.92 (m, 6H).

### 12. Synthesis of LP12

The synthesis of LP12 is shown below:

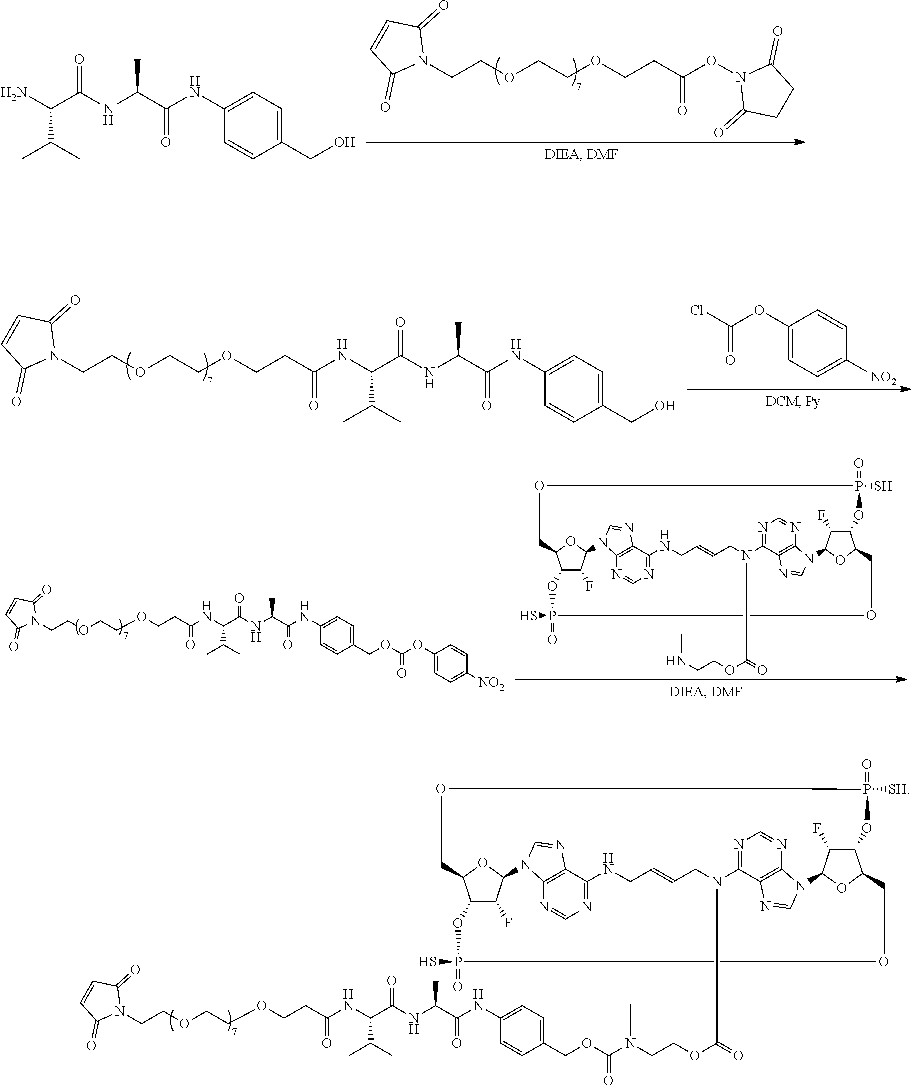

### Synthesis of 1-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]-3,6,9,12,15,18,21,24-octaoxaheptacosan-27-amide

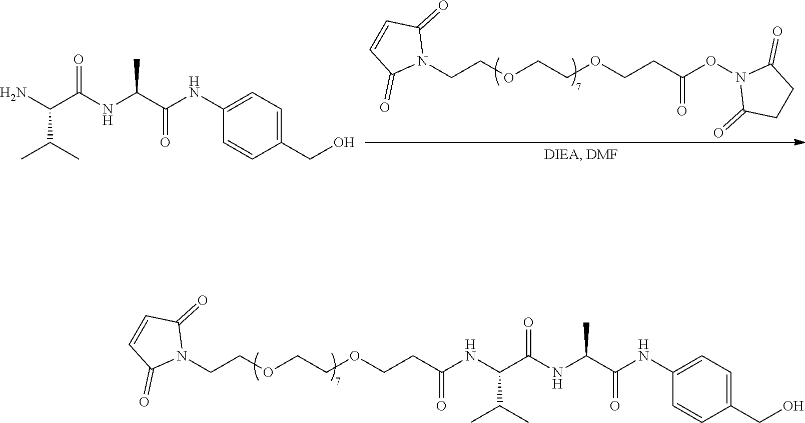

A solution of (2S)-2-amino-N-[(1S)-1-{[4-(hydroxymethyl)phenyl]carbamoyl}ethyl]-3-methylbutanamide (100 mg, 0.341 mmol), 2,5-dioxopyrrolidin-1-yl 1-(2,5-dioxopyrrol-1-yl)-3,6,9,12,15,18,21,24-octaoxaheptacosan-27-oate (210.87 mg, 0.341 mmol) and DIEA (88.11 mg, 0.682 mmol) in DMF (3 mL) was stirred at room temperature for 2 h. The resulting mixture was concentrated under reduced pressure. The residue was purified by Prep-TLC (DCM/MeOH 8:1) to afford 1-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]-3,6,9,12,15,18,21,24-octaoxaheptacosan-27-amide (180 mg) as a white solid. LC-MS (ESI): 797.4 [M+H]⁺.

### Synthesis of {4-[(2S)-2-[(2S)-2-[1-(2,5-dioxo-2,5-dihydro-1H-pyrrol-1-yl)-3,6,9,12,15,18,21,24-octaoxaheptacosan-27-amido]-3-methylbutanamido]propanamido]phenyl}methyl 4-nitrophenyl carbonate

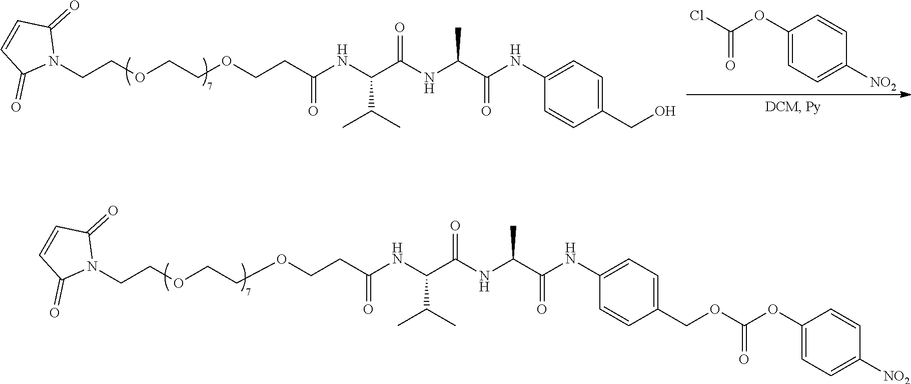

Pyridine (44.67 mg, 0.565 mmol) was added to a solution of 1-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]-3,6,9,12,15,18,21,24-octaoxaheptacosan-27-amide (180 mg, 0.226 mmol) and 4-nitrophenyl carbonochloridate (113.82 mg, 0.565 mmol) in DCM (2 mL) at room temperature. The solution was stirred at room temperature for 3 h. The resulting mixture was concentrated under reduced pressure. The residue was purified by Prep-TLC (DCM/IPA 10:1) to afford {4-[(2S)-2-[(2S)-2-[1-(2,5-dioxo-2,5-dihydro-1H-pyrrol-1-yl)-3,6,9,12,15,18,21,24-octaoxaheptacosan-27-amido]-3-methylbutanamido]propanamido]phenyl}methyl 4-nitrophenyl carbonate (109 mg) as a light-yellow oil. LC-MS (ESI): 962.4 [M+H]⁺.

### Synthesis of 2-{[({4-[(2S)-2-[(2S)-2-[1-(2,5-dioxo-2,5-dihydro-1H-pyrrol-1-yl)-3,6,9,12,15,18,21,24-octaoxaheptacosan-27-amido]-3-methylbutanamido]propanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP12)

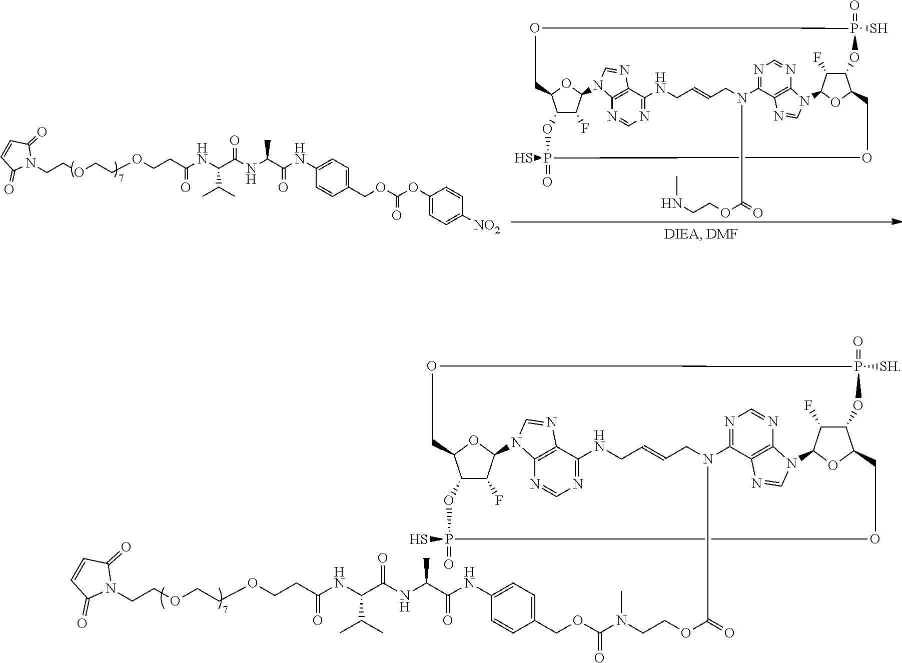

DIEA (47.02 mg, 0.365 mmol) was added to a solution of 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (61.68 mg, 0.073 mmol) and {4-[(2S)-2-[(2S)-2-[1-(2,5-dioxo-2,5-dihydro-1H-pyrrol-1-yl)-3,6,9,12,15,18,21,24-octaoxaheptacosan-27-amido]-3-methylbutanamido]propanamido]phenyl}methyl 4-nitrophenyl carbonate (70 mg, 0.073 mmol) in DMF (2.5 mL) at room temperature. The solution was stirred at room temperature for 3 h. The resulting mixture was concentrated under reduced pressure. The residue was purified by Prep-TLC (DCM/MeOH 6:1) and Prep-HPLC (Column: XBridge Prep OBD C18 Column, 19×250 mm, 5 μm; Mobile Phase A: Water(50 mmol HCO₂NH₄), Mobile Phase B: MeCN; Flow rate: 25 mL/min; Gradient: 25% B to 35% B in 10 min, 35% B; Wavelength: 220/254 nm) to afford 2-{[({4-[(2S)-2-[(2S)-2-[1-(2,5-dioxo-2,5-dihydro-1H-pyrrol-1-yl)-3,6,9,12,15,18,21,24-octaoxaheptacosan-27-amido]-3-methylbutanamido]propanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP12) (23.5 mg) as a white solid.

LC-MS (ESI): 1671.70 [M+H]⁺.

¹H NMR (400 MHz, Methanol-d₄) δ 8.92 (s, 1H), 8.82-8.66 (m, 2H), 8.30-7.99 (m, 2H), 7.71-7.53 (m, 2H), 7.37-7.28 (m, 1H), 7.26-7.14 (m, 1H), 6.84 (s, 2H), 6.56-6.22 (m, 2H), 6.08-5.86 (m, 1H), 5.84-5.23 (m, 4H), 5.13-4.94 (m, 2H), 4.76-4.40 (m, 7H), 4.37-4.13 (m, 3H), 4.09-3.89 (m, 2H), 3.81-3.74 (m, 2H), 3.72-3.68 (m, 2H), 3.67-3.56 (m, 24H), 2.83-2.69 (m, 3H), 2.65-2.55 (m, 2H), 2.30-2.04 (m, 1H), 1.54-1.40 (m, 3H), 1.08-0.91 (m, 6H).

### 13. Synthesis of LP13

The synthesis of LP13 is shown below:

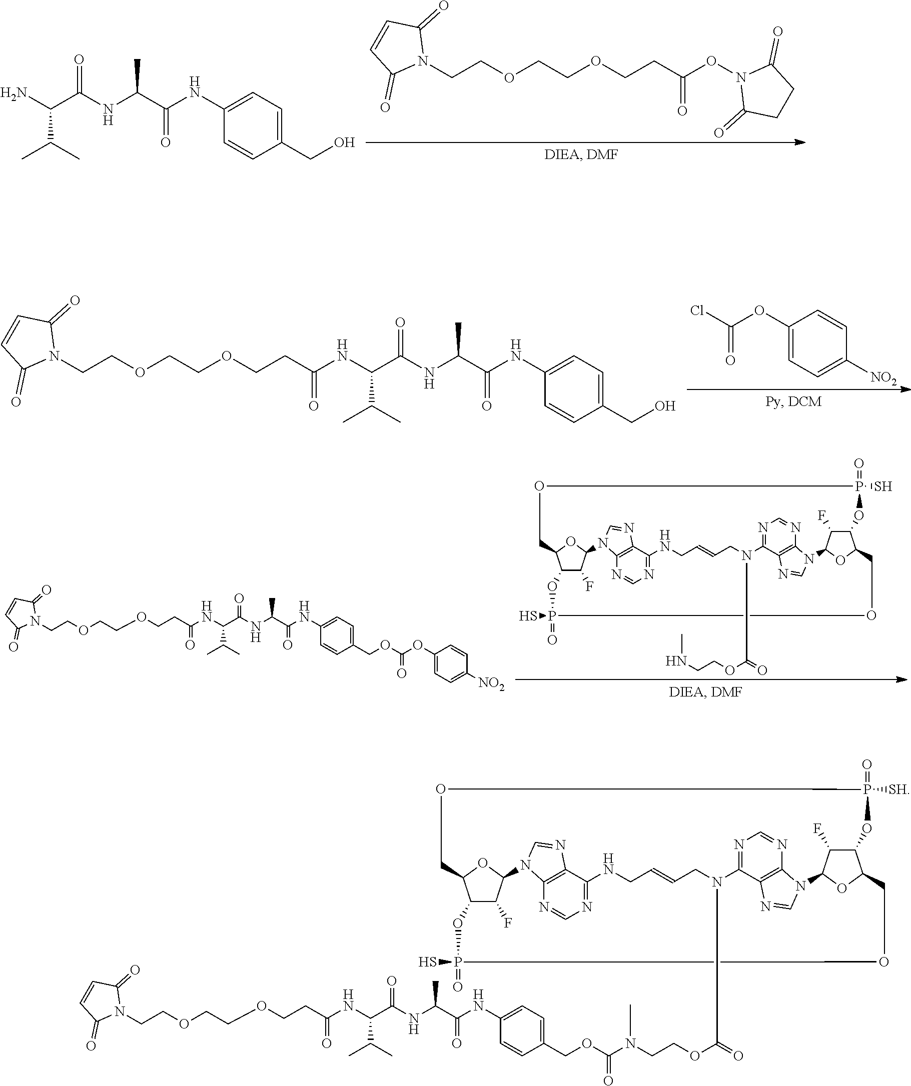

### Synthesis of (2S)-2-(3-{2-[2-(2,5-dioxo-2,5-dihydro-1H-pyrrol-1-yl)ethoxy]ethoxy}propanamido)-N-[(1S)-1-{[4-(hydroxymethyl)phenyl]carbamoyl}ethyl]-3-methylbutanamide

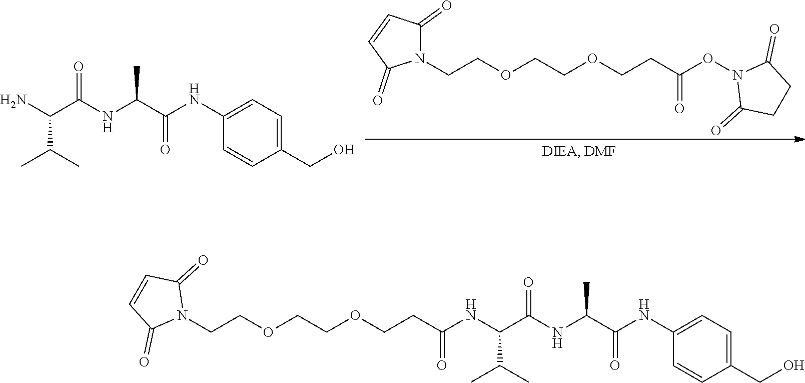 [SMILES: CC(C)[C@H](N)C(=O)N[C@@H](C)C(=O)Nc1ccc(CO)cc1 ; O=C(CCOCCOCCN1C(=O)C=CC1=O)ON1C(=O)CCC1=O ; CC(C)[C@H](NC(=O)CCOCCOCCN1C(=O)C=CC1=O)C(=O)N[C@@H](C)C(=O)Nc1ccc(CO)cc1]

To a mixture of (2S)-2-amino-N-[(1S)-1-{[4-(hydroxymethyl)phenyl]carbamoyl}ethyl]-3-methylbutanamide (200 mg, 0.682 mmol) and 2,5-dioxopyrrolidin-1-yl 3-{2-[2-(2,5-dioxopyrrol-1-yl)ethoxy]ethoxy}propanoate (241.55 mg, 0.682 mmol) in DMF(5 mL) was added DIEA (264.34 mg, 2.046 mmol) at 0° C. The resulting mixture was stirred at room temperature for 2 h. The resulting mixture was concentrated under vacuum. The residue was purified by silica gel column chromatography, eluted with DCM/MeOH (10:1) to afford (2S)-2-(3-{2-[2-(2,5-dioxo-2,5-dihydro-1H-pyrrol-1-yl)ethoxy]ethoxy}propanamido)-N-[(1S)-1-{[4-(hydroxymethyl)phenyl]carbamoyl}ethyl]-3-methylbutanamide (170 mg) as a white solid. LC-MS (ESI): 533.3 [M+H]⁺.

### Synthesis of {4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methyl 4-nitrophenyl carbonate

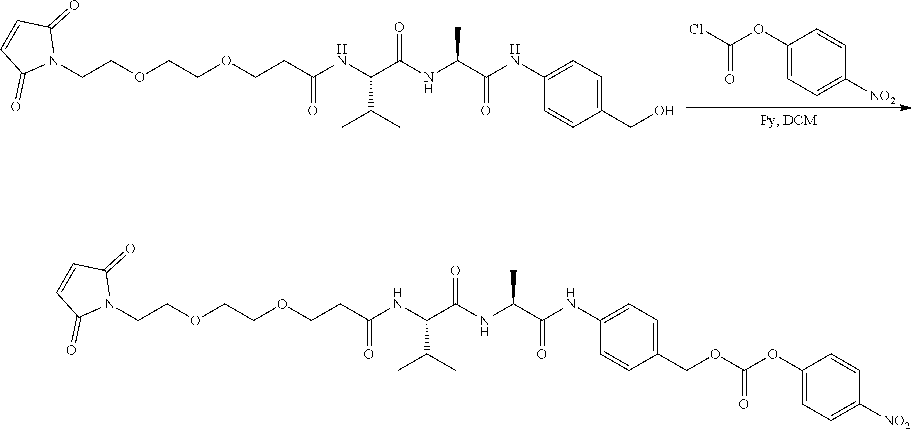 [SMILES: CC(C)[C@H](NC(=O)CCOCCOCCN1C(=O)C=CC1=O)C(=O)N[C@@H](C)C(=O)Nc1ccc(CO)cc1 ; O=C(Cl)Oc1ccc([N+](=O)[O-])cc1 ; CC(C)[C@H](NC(=O)CCOCCOCCN1C(=O)C=CC1=O)C(=O)N[C@@H](C)C(=O)Nc1ccc(COC(=O)Oc2ccc([N+](=O)[O-])cc2)cc1]

To a mixture of (2S)-2-(3-{2-[2-(2,5-dioxo-2,5-dihydro-1H-pyrrol-1-yl)ethoxy]ethoxy}propanamido)-N-[(1S)-1-{[4-(hydroxymethyl)phenyl]carbamoyl}ethyl]-3-methylbutanamide (150 mg, 0.282 mmol) and 4-nitrophenyl carbonochloridate (113.53 mg, 0.564 mmol) in DCM (5 mL) was added Pyridine (33.42 mg, 0.423 mmol) at 0° C. The resulting mixture was stirred at 25° C. for 4 h. The resulting mixture was concentrated under reduced pressure. The residue was purified by Prep-TLC (CH₂Cl₂/IPA 10:1) to afford {4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methyl 4-nitrophenyl carbonate (70 mg) as a white solid.

LC-MS (ESI): 698.4[M+H]⁺.

### Synthesis of 2-{[({4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP13)

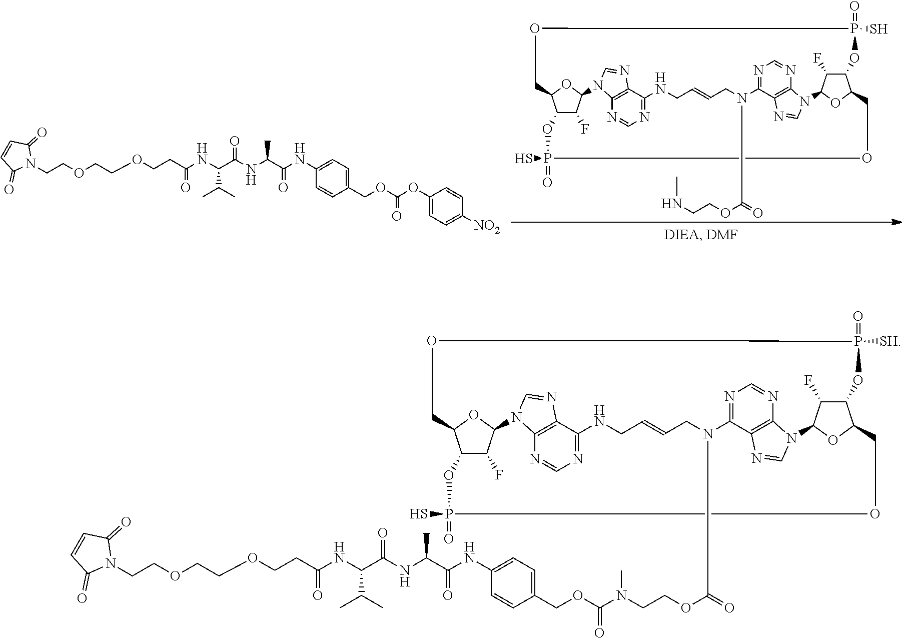 [SMILES: CC(C)[C@H](NC(=O)CCOCCOCCN1C(=O)C=CC1=O)C(=O)N[C@@H](C)C(=O)Nc1ccc(COC(=O)Oc2ccc([N+](=O)[O-])cc2)cc1 ; CNCCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32 ; CC(C)[C@H](NC(=O)CCOCCOCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of {4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methyl 4-nitrophenyl carbonate (36 mg, 0.052 mmol) and 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (43.74 mg, 0.052 mmol) in DMF (2 mL) was added DIEA (33.34 mg, 0.260 mmol) at room temperature. The resulting mixture was stirred at room temperature overnight. The resulting mixture was concentrated under vacuum. The residue was purified by Prep-TLC(EtOAc/MeOH 3:1) and Prep-HPLC with the following conditions (Column: XBridge Prep Phenyl OBD Column, 19×250 mm, 5 μm; Mobile Phase A: Water(50 mmol/L HCO₂NH₄), Mobile Phase B: MeCN; Flow rate: 25 mL/min; Gradient: 35% B to 60% B in 15 min, 60% B; Wavelength: 254 nm to afford 2-{[({4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP13) (3.5 mg) as a white solid.

LC-MS (ESI): 1406.40 [M+H]⁺.

¹H NMR (400 MHz, Methanol-d₄) δ 8.92 (s, 1H), 8.72 (m, 2H), 8.31-8.02 (m, 2H), 7.60 (m, 2H), 7.31 (m, 1H), 7.21 (m, 1H), 6.82 (s, 2H), 6.43 (m, 1H), 6.32 (m, 1H), 6.02 (s, 1H), 5.84 (m, 1H), 5.51 (m, 1H), 5.02 (s, 2H), 4.92 (m, 4H), 4.62 (m, 4H), 4.54-4.45 (m, 3H), 4.42 (s, 2H), 4.35-4.13 (m, 2H), 4.09 (m, 4H), 3.84 (m, 4H), 3.79-3.61 (m, 3H), 3.42 (m, 4H), 2.9 (m, 2H), 2.73 (m, 3H), 2.55 (m, 2H), 2.24-2.07 (m, 1H), 1.50-1.39 (m, 3H), 1.00 (m, 6H).

### 14. Synthesis of LP14

The synthesis of LP14 is shown below:

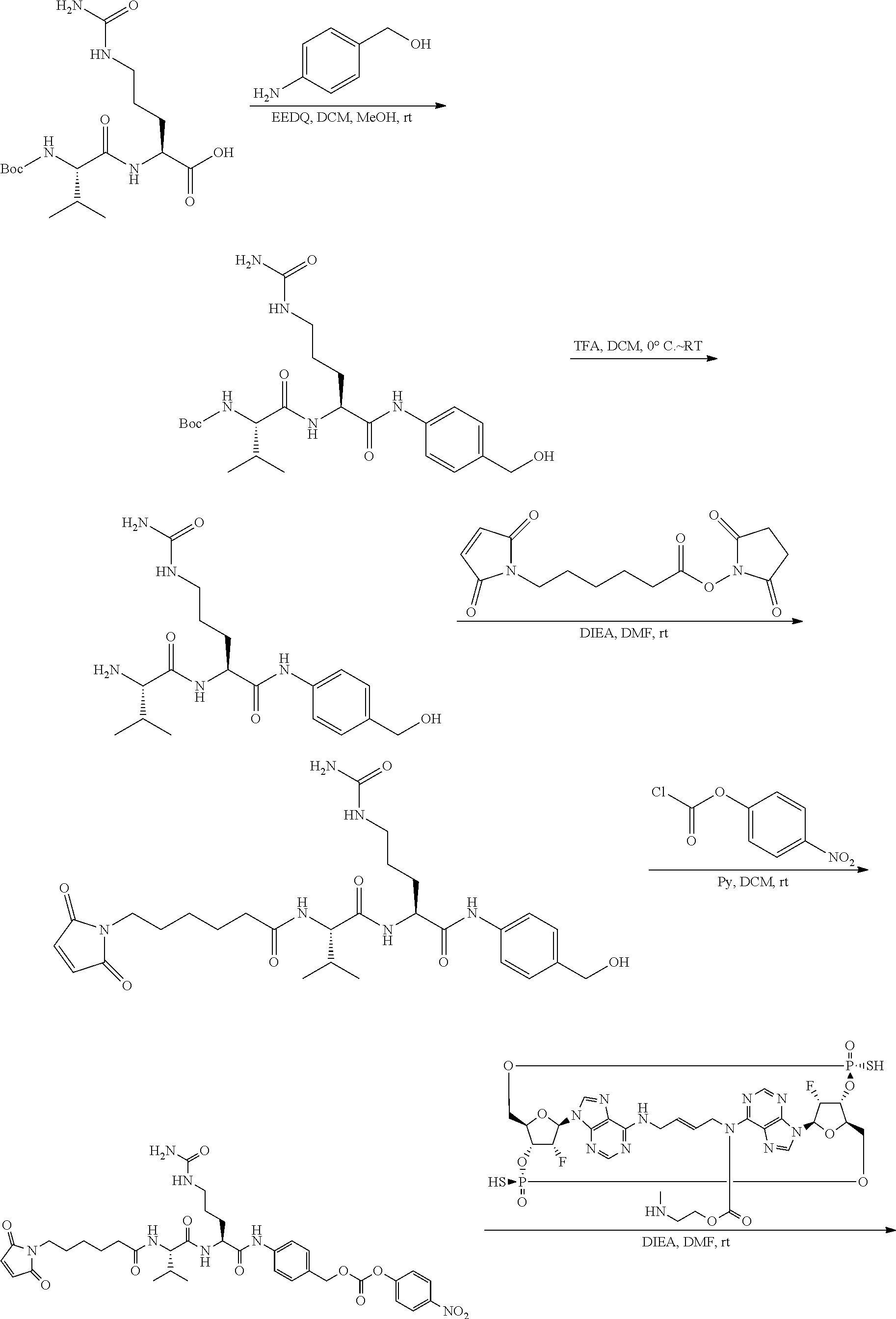

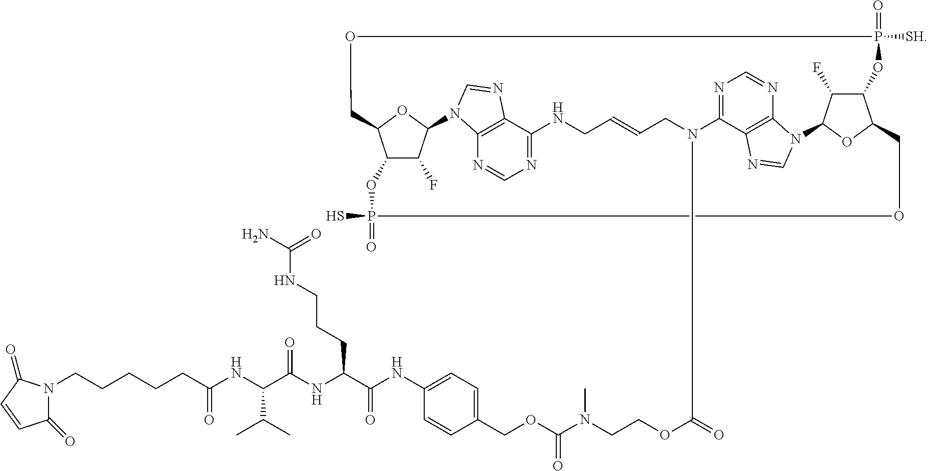 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of tert-butyl N-[(1S)-1-{[(1S)-4-(carbamoylamino)-1-{[4-(hydroxymethyl)phenyl]carbamoyl}butyl]carbamoyl}-2-methylpropyl]carbamate

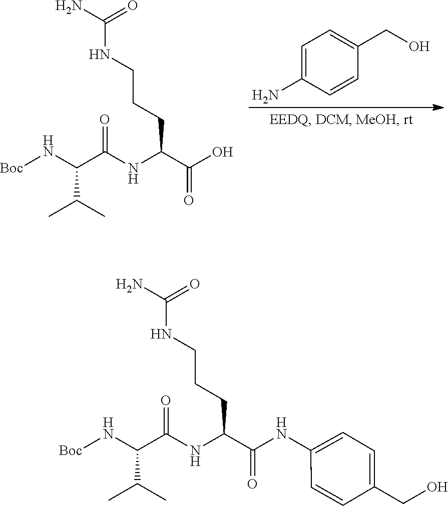 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1]

Ethyl 2-ethoxy-1,2-dihydroquinoline-1-carboxylate (2.64 g, 10.682 mmol) was added to a mixture of (25)-2-[(25)-2-[(tert-butoxycarbonyl)amino]-3-methylbutanamido]-5-(carbamoylamino)pentanoic acid (2 g, 5.341 mmol) and p-aminobenzylalcohol (657.81 mg, 5.341 mmol) in DCM (12 mL) and MeOH (2 mL) at room temperature. The resulting mixture was stirred at 25° C. for 2 h. The resulting mixture was concentrated under reduced pressure. The residue was purified by silica gel column chromatography, eluted with DCM/MeOH (5:1) to afford tert-butyl N-[(15)-1-{[(15)-4-(carbamoylamino)-1-{[4-(hydroxymethyl)phenyl]carbamoyl}butyl]carbamoyl}-2-methylpropyl]carbamate (1.8 g) as a light pink solid. LC-MS (ESI): 480.5 [M+H]⁺.

### Synthesis of (2S)-2-[(2S)-2-amino-3-methylbutanamido]-5-(carbamoylamino)-N-[4-(hydroxymethyl)phenyl]pentanamide

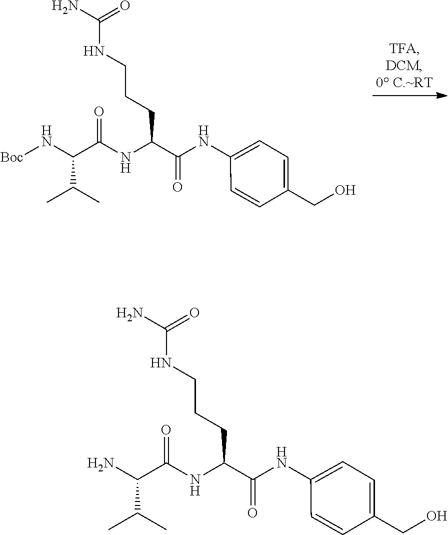 [SMILES: CC(C)[C@H](NC(=O)OC(C)(C)C)C(=O)N[C@@H](CCCNC(N)=O)C(=O)Nc1ccc(CO)cc1 ; CC(C)[C@H](N)C(=O)N[C@@H](CCCNC(N)=O)C(=O)Nc1ccc(CO)cc1]

TFA (10 mL) was added to a mixture of tert-butyl N-[(1S)-1-{[(1S)-4-(carbamoylamino)-1-{[4-(hydroxymethyl)phenyl]carbamoyl}butyl]carbamoyl}-2-methylpropyl]carbamate (1.8 g, 3.753 mmol) in DCM (10 mL) at 0° C. The resulting mixture was stirred at 25° C. for 30 min. The resulting mixture was concentrated under reduced pressure. Then K₂CO₃ (2.07 g, 15.012 mmol) was added to a mixture of above intermediate in MeOH (5 mL), THF (5 mL) and H₂O (5 mL) at 0° C. The resulting mixture was stirred at 25° C. for 2 h. The resulting mixture was concentrated under reduced pressure. The residue was purified by silica gel column chromatography, eluted with DCM/MeOH (3:1) to afford (2S)-2-[(2S)-2-amino-3-methylbutanamido]-5-(carbamoylamino)-N-[4-(hydroxymethyl)phenyl]pentanamide (1.3 g) as a white solid. LC-MS (ESI): 380.4 [M+H]⁺.

### Synthesis of N-[(1S)-1-{[(1S)-4-(carbamoylamino)-1-{[4-(hydroxymethyl)phenyl]carbamoyl}butyl]carbamoyl}-2-methylpropyl]-6-(2,5-dioxopyrrol-1-yl)hexanamide

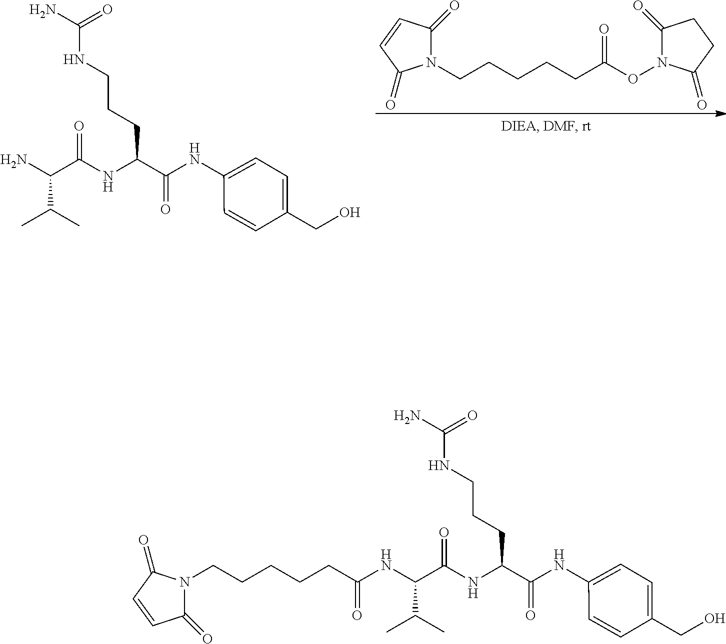 [SMILES: CC(C)[C@H](N)C(=O)N[C@@H](CCCNC(N)=O)C(=O)Nc1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](CCCNC(N)=O)C(=O)Nc1ccc(CO)cc1]

DIEA (1.33 g, 10.278 mmol) was added to a mixture of (2S)-2-[(2S)-2-amino-3-methylbutanamido]-5-(carbamoylamino)-N-[4-(hydroxymethyl)phenyl]pentanamide (1.3 g, 3.426 mmol) and 2,5-dioxopyrrolidin-1-yl 6-(2,5-dioxopyrrol-1-yl)hexanoate (1.06 g, 3.426 mmol) in DMF (5 mL) at 25° C. The resulting mixture was stirred at 25° C. for 3 h. The residue was purified by reversed-phase flash chromatography with the following conditions: column, C18 silica gel; mobile phase A: Water (0.1% FA), mobile phase B MeCN, 10% to 35% gradient in 30 min; detector, UV 254 nm. This resulted in N-[(1S)-1-{[(1S)-4-(carbamoylamino)-1-{[4-(hydroxymethyl)phenyl]carbamoyl}butyl]carbamoyl}-2-methylpropyl]-6-(2,5-dioxopyrrol-1-yl)hexanamide (1.5 g) as a white solid. LC-MS (ESI): 573.6 [M+H]⁺.

### Synthesis of {4-[(2S)-5-(carbamoylamino)-2-[(2S)-2-[6-(2,5-dioxopyrrol-1-yl)hexanamido]-3-methylbutanamido]pentanamido]phenyl}methyl 4-nitrophenyl carbonate

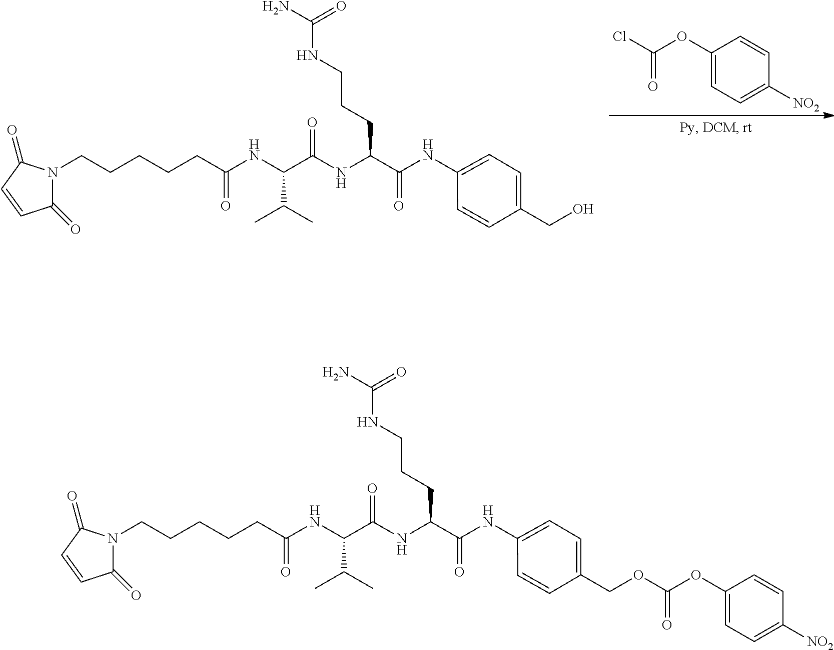 [SMILES: CC(C)[C@H](NC(=O)CCCCCN1C(=O)C=CC1=O)C(=O)N[C@@H](CCCNC(N)=O)C(=O)Nc1ccc(CO)cc1 ; O=C(Cl)Oc1ccc([N+](=O)[O-])cc1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

Pyridine (82.88 mg, 1.048 mmol) was added to a mixture of N-[(1S)-1-{[(1S)-4-(carbamoylamino)-1-{[4-(hydroxymethyl)phenyl]carbamoyl}butyl]carbamoyl}-2-methylpropyl]-6-(2,5-dioxopyrrol-1-yl)hexanamide (300 mg, 0.524 mmol) and 4-nitrophenyl carbonochloridate (211.18 mg, 1.048 mmol) in DCM (5 mL) at 25° C. The resulting mixture was stirred at 25° C. for 14 h. The resulting mixture was concentrated under reduced pressure. The residue was purified by reversed-phase flash chromatography with the following conditions: column, C18 silica gel; mobile phase A: Water (0.1% FA), mobile phase B: MeCN, 20% to 50% gradient in 20 min; detector, UV 254 nm. This resulted in {4-[(2S)-5-(carbamoylamino)-2-[(2S)-2-[6-(2,5-dioxopyrrol-1-yl)hexanamido]-3-methylbutanamido]pentanamido]phenyl}methyl 4-nitrophenyl carbonate (110 mg) as a yellow solid.

LC-MS (ESI): 738.7 [M+H]⁺

### Synthesis of 2-{[({4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}methoxy)carbonyl](methyl)amino}ethyl(1R,3R,15E,28R,2 9R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP14)

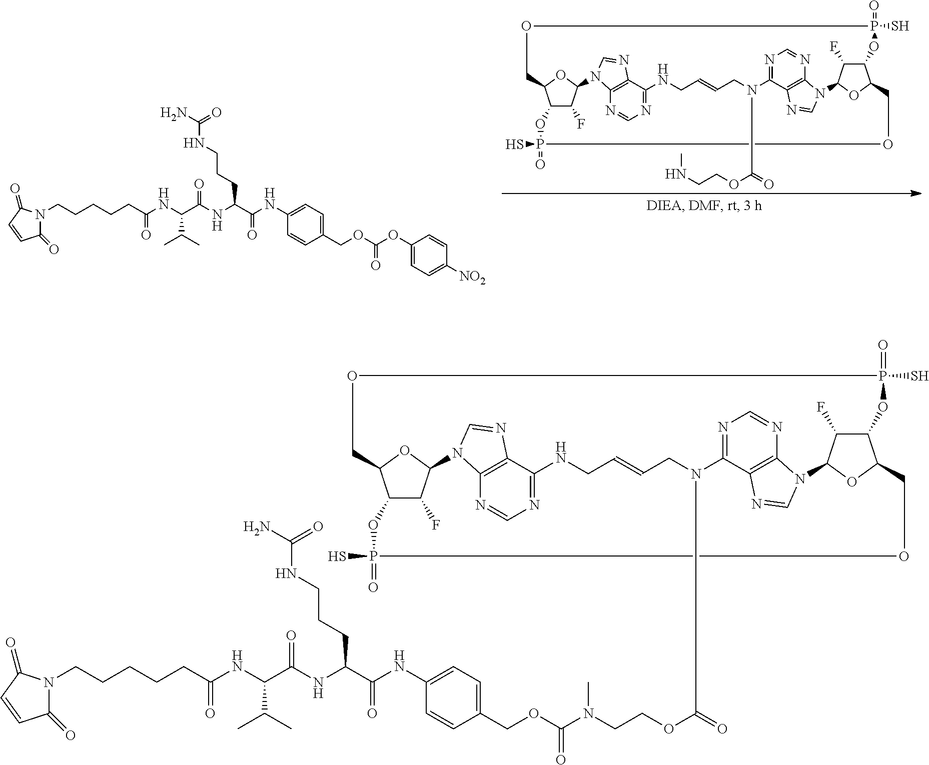 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CNCCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32 ; CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

DIEA (26.28 mg, 0.205 mmol) was added to a mixture of {4-[(2S)-5-(carbamoylamino)-2-[(2S)-2-[6-(2,5-dioxopyrrol-1-yl)hexanamido]-3-methylbutanamido]pentanamido]phenyl}methyl 4-nitrophenyl carbonate (30 mg, 0.041 mmol) and 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (34.47 mg, 0.041 mmol) in DMF (1 mL) under nitrogen atmosphere. The resulting mixture was stirred at 25° C. under nitrogen atmosphere for 3 h. The resulting mixture was concentrated under reduced pressure. The residue was purified by Prep-TLC (EtOAc (1% TEA)/MeOH 4:1). The crude product (30 mg) was purified by Prep-HPLC with the following conditions (Column: XBridge Prep OBD C18 Column, 19×250 mm, 5 μm; Mobile Phase A: Water(50 mmol/L HCO₂NH₄), Mobile Phase B: MeCN; Flow rate: 25 mL/min; Gradient: 20% B to 30% B in 10 min, 30% B; Wavelength: 220 nm to afford 2-{[({4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP14) (6.3 mg) as a white solid.

LC-MS (ESI): 1446.45 [M+H]⁺.

¹H NMR (300 MHz, Methanol-d₄) δ 8.95-8.68 (m, 3H), 8.57-8.53 (m, 3H), 8.09 (s, 1H), 7.64-7.57 (m, 2H), 7.39-7.16 (m, 2H), 6.80 (s, 1H), 6.50-6.24 (m, 2H), 6.11-5.66 (m, 2H), 5.62-5.25 (m, 2H), 4.36-4.11 (m, 3H), 4.10-3.91 (m, 2H), 3.67 (s, 2H), 3.58-3.44 (m, 3H), 2.88 (s, 1H), 2.76 (d, J=15.0 Hz, 2H), 2.31-2.20 (m, 3H), 2.14-1.73 (m, 4H), 1.72-1.49 (m, 8H), 1.07-0.78 (m, 16H).

### 15. Synthesis of LP15

The synthesis of LP15 is shown below:

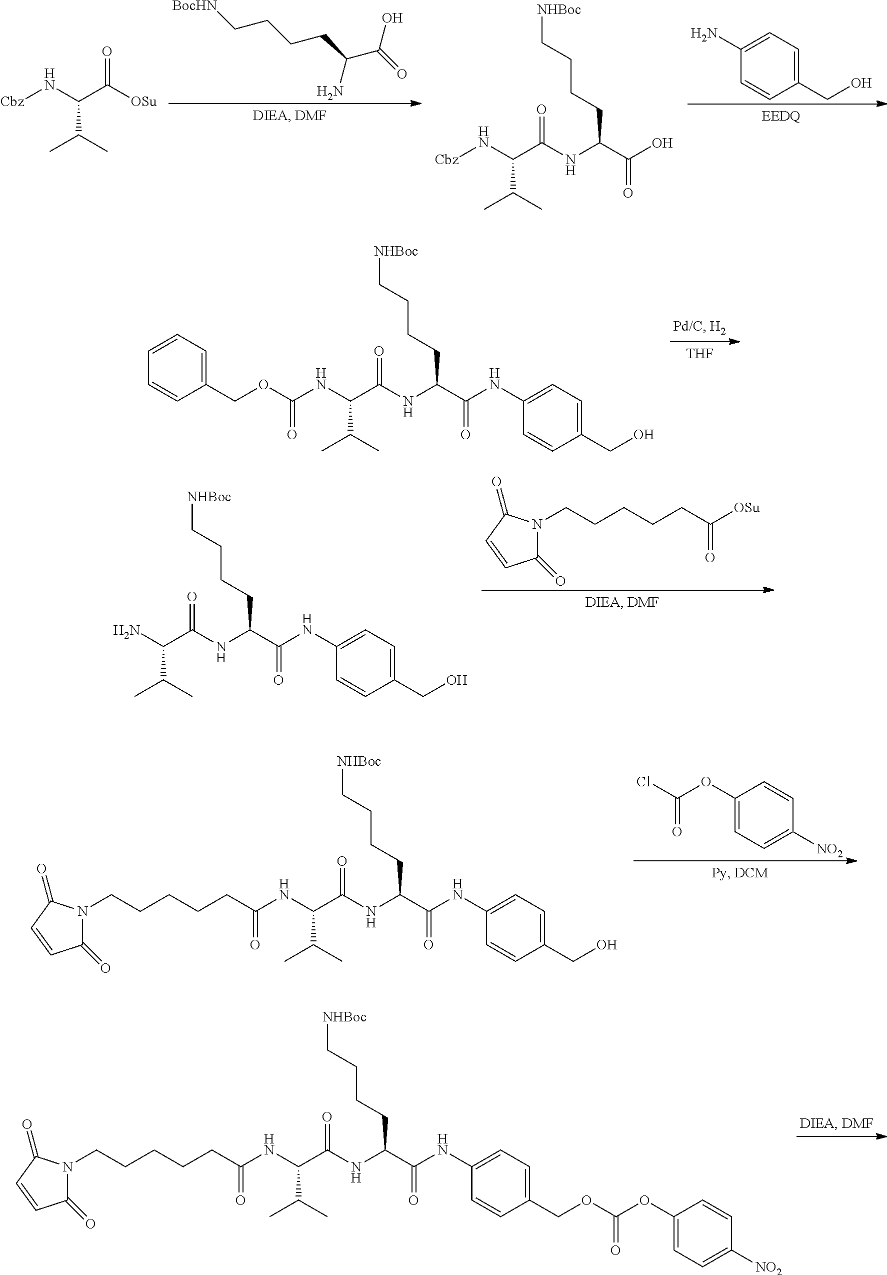

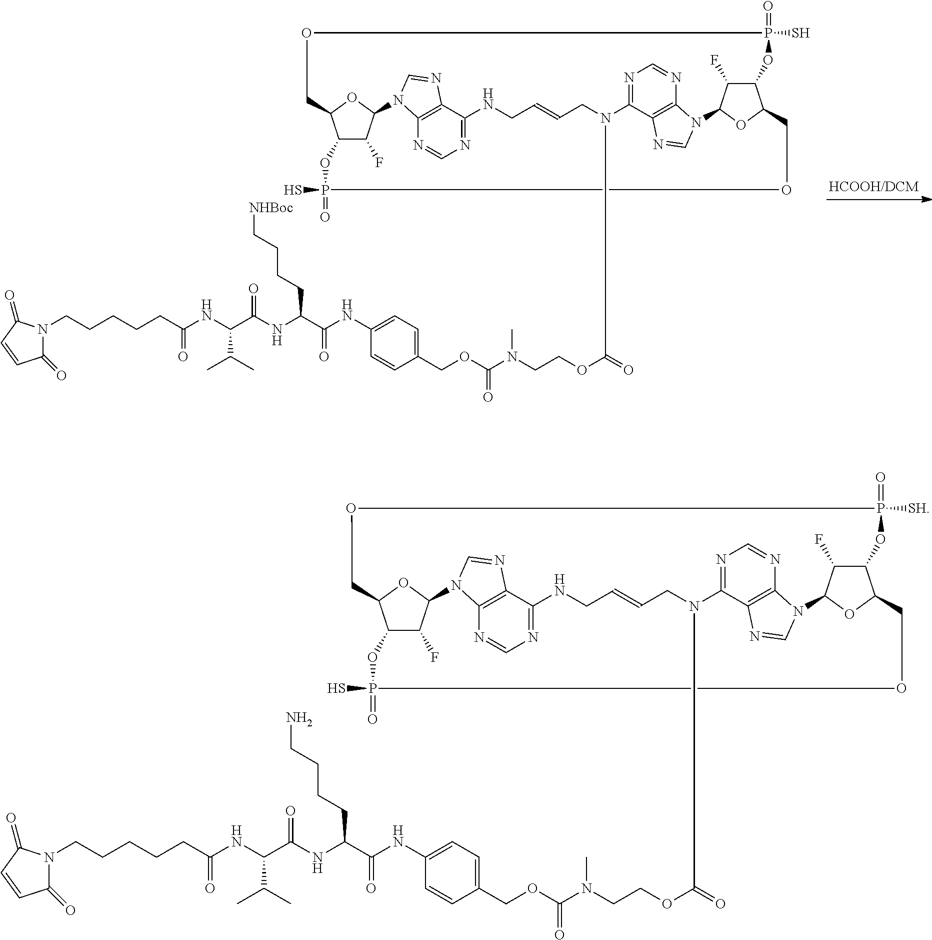 [SMILES: CC(C)[C@H](NC(=O)CCCCCN1C(=O)C=CC1=O)C(=O)N[C@@H](CCCCNC(=O)OC(C)(C)C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](NC(=O)CCCCCN1C(=O)C=CC1=O)C(=O)N[C@@H](CCCCN)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of (2S)-2-[(2S)-2-{[(benzyloxy)carbonyl]amino}-3-methylbutanamido]-6-[(tert-butoxycarbonyl)amino]hexanoic acid

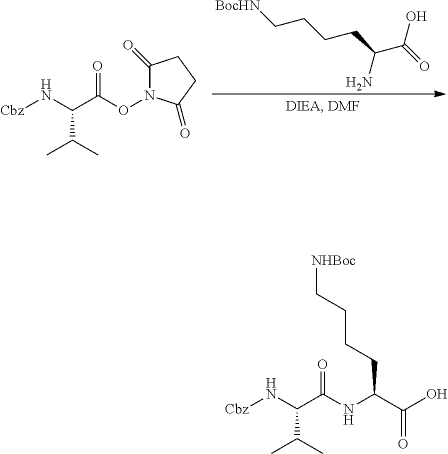 [SMILES: CC(C)[C@H](NC(=O)OCc1ccccc1)C(=O)ON1C(=O)CCC1=O ; CC(C)(C)OC(=O)NCCCC[C@H](N)C(=O)O ; CC(C)[C@H](NC(=O)OCc1ccccc1)C(=O)N[C@@H](CCCCNC(=O)OC(C)(C)C)C(=O)O]

To a mixture of 2,5-dioxopyrrolidin-1-yl (2S)-2-{[(benzyloxy)carbonyl]amino}-3-methylbutanoate (1 g, 2.871 mmol) and (2S)-2-amino-6-[(tert-butoxycarbonyl)amino]hexanoic acid (707.06 mg, 2.871 mmol) in DMF (5 mL) was added DIEA (1.11 g, 8.613 mmol) at room temperature.

The resulting mixture was stirred at room temperature overnight. The solution was purified by reversed-phase flash chromatography with the following conditions: Column: C18; mobile phase A: Water (0.5% FA), mobile phase B: MeCN; gradient: 20% B to 50% B in 30 min; 220/254 nm. This resulted in (2S)-2-[(2S)-2-{[(benzyloxy)carbonyl]amino}-3-methylbutanamido]-6-[(tert-butoxycarbonyl)amino]hexanoic acid (1 g) as an off-white solid. LC-MS (ESI): 480.6 [M+H]⁺

### Synthesis of Benzyl N-[(1S)-1-{[(1S)-5-{[(tert-butoxy)carbonyl]amino}-1-{[4-(hydroxymethyl)phenyl]carbamoyl}pentyl]carbamoyl}-2-methylpropyl]carbamate

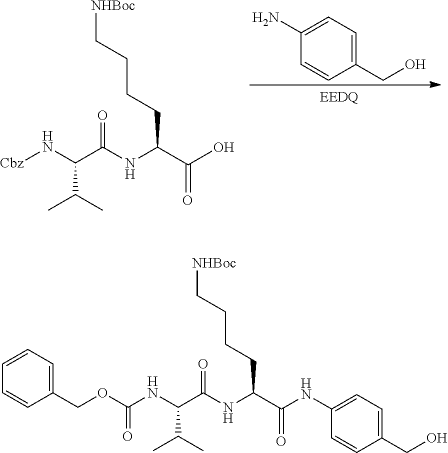 [SMILES: CC(C)[C@H](NC(=O)OCc1ccccc1)C(=O)N[C@@H](CCCCNC(=O)OC(C)(C)C)C(=O)O ; Nc1ccc(CO)cc1 ; CC(C)[C@H](NC(=O)OCc1ccccc1)C(=O)N[C@@H](CCCCNC(=O)OC(C)(C)C)C(=O)Nc1ccc(CO)cc1]

To a mixture of (2S)-2-[(2S)-2-{[(benzyloxy)carbonyl]amino}-3-methylbutanamido]-6-[(tert-butoxycarbonyl)amino]hexanoic acid (1 g, 2.085 mmol) and p-aminobenzylalcohol (256.80 mg, 2.085 mmol) in DCM (6 mL)/MeOH (1 mL) were added ethyl 2-ethoxy-1,2-dihydroquinoline-1-carboxylate (515.64 mg, 2.085 mmol). The resulting mixture was stirred at room temperature overnight. The resulting mixture was concentrated under reduced pressure. The residue was purified by silica gel column chromatography, eluted with DCM/MeOH (10:1) to afford Benzyl N-[(1S)-1-{[(1S)-5-{[(tert-butoxy)carbonyl]amino}-1-{[4-(hydroxymethyl)phenyl]carbamoyl}pentyl]carbamoyl}-2-methylpropyl]carbamate (1 g) as a white solid. LC-MS (ESI): 585.3 [M+H]⁺

### Synthesis of tert-butyl N-[(5S)-5-[(2S)-2-amino-3-methylbutanamido]-5-{[4-hydroxymethyl)phenyl]carbamoyl}pentyl]carbamate

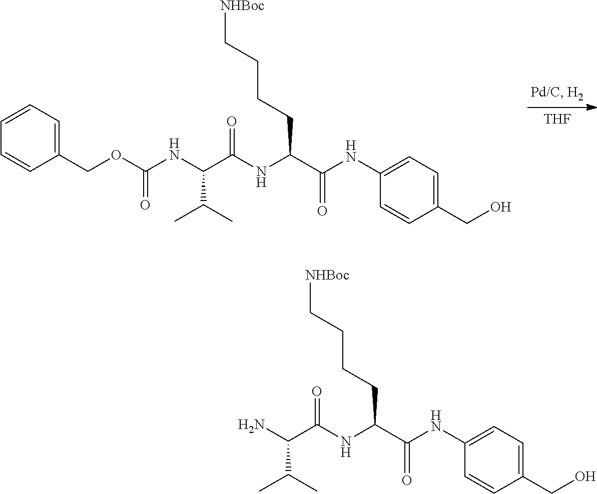 [SMILES: CC(C)[C@H](NC(=O)OCc1ccccc1)C(=O)N[C@@H](CCCCNC(=O)OC(C)(C)C)C(=O)Nc1ccc(CO)cc1 ; CC(C)[C@H](N)C(=O)N[C@@H](CCCCNC(=O)OC(C)(C)C)C(=O)Nc1ccc(CO)cc1 ; "THF"]

To a mixture of Benzyl N-[(1S)-1-{[(1S)-5-{[(tert-butoxy)carbonyl]amino}-1-{[4-(hydroxymethyl)phenyl]carbamoyl}pentyl]carbamoyl}-2-methylpropyl]carbamate (1 g, 1.710 mmol) in THF(10 mL) was added Pd/C (200 mg) at room temperature. The resulting mixture was stirred at room temperature overnight under hydrogen atmosphere. The resulting mixture was filtered, the filter cake was washed with THF. The filtrate was concentrated under reduced pressure. This resulted in tert-butyl N-[(5S)-5-[(2S)-2-amino-3-methylbutanamido]-5-{[4-(hydroxymethyl)phenyl]carbamoyl}pentyl]carbamate (800 mg) as a green solid. LC-MS (ESI): 451.3 [M+H]⁺.

### Synthesis of tert-butyl N-[(5S)-5-[(2S)-2-[6-(2,5-dioxo-2,5-dihydro-1H-pyrrol-1-yl)hexanamido]-3-methylbutanamido]-5-{[4-(hydroxymethyl)phenyl]carbamoyl}pentyl]carbamate

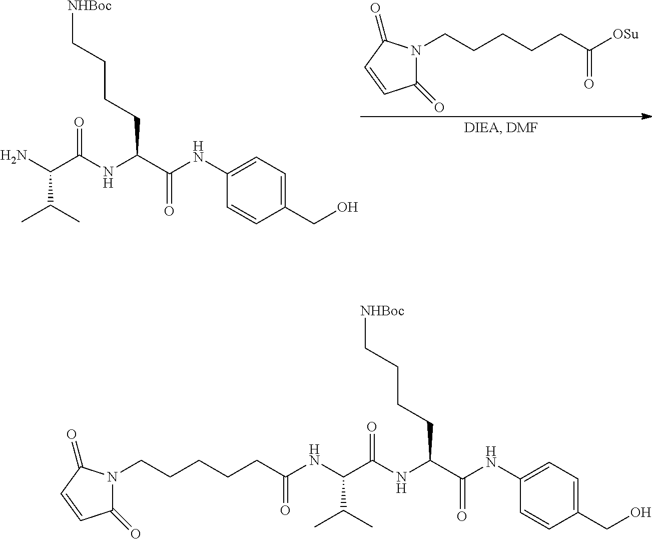

To a mixture of tert-butyl N-[(5S)-5-[(2S)-2-amino-3-methylbutanamido]-5-{[4-(hydroxymethyl)phenyl]carbamoyl}pentyl]carbamate (800 mg, 1.775 mmol) and 2,5-dioxopyrrolidin-1-yl 6-(2,5-dioxopyrrol-1-yl)hexanoate (68.42 mg, 0.222 mmol) in DMF (5 mL) was added DIEA (688.43 mg, 5.325 mmol) at room temperature. The resulting mixture was stirred at room temperature for 1 h. The resulting mixture was concentrated under reduced pressure. The residue was purified by silica gel column chromatography, eluted with DCM/MeOH (10:1) to afford tert-butyl N-[(5S)-5-[(2S)-2-[6-(2,5-dioxo-2,5-dihydro-1H-pyrrol-1-yl)hexanamido]-3-methylbutanamido]-5-{[4-(hydroxymethyl)phenyl]carbamoyl}pentyl]carbamate (650 mg) as an off-white solid. LC-MS (ESI): 644.5 [M+H]⁺.

### Synthesis of {4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methyl 4-nitrophenyl carbonate

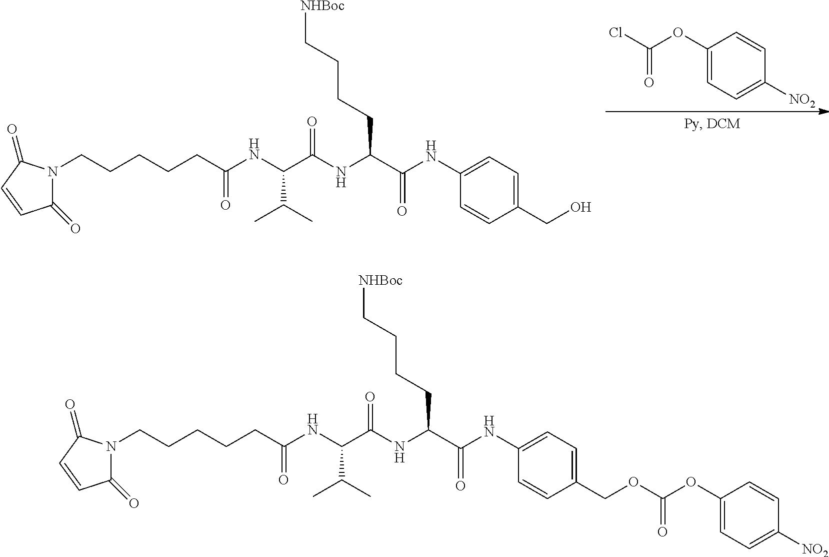 [SMILES: CC(C)[C@H](NC(=O)CCCCCN1C(=O)C=CC1=O)C(=O)N[C@@H](CCCCNC(=O)OC(C)(C)C)C(=O)Nc1ccc(CO)cc1 ; O=C(Cl)Oc1ccc([N+](=O)[O-])cc1 ; CC(C)[C@H](NC(=O)CCCCCN1C(=O)C=CC1=O)C(=O)N[C@@H](CCCCNC(=O)OC(C)(C)C)C(=O)Nc1ccc(COC(=O)Oc2ccc([N+](=O)[O-])cc2)cc1]

To a mixture of tert-butyl N-[(5S)-5-[(2S)-2-[6-(2,5-dioxo-2,5-dihydro-1H-pyrrol-1-yl)hexanamido]-3-methylbutanamido]-5-{[4-(hydroxymethyl)phenyl]carbamoyl}pentyl]carbamate (100 mg, 0.155 mmol) and 4-nitrophenyl carbonochloridate (62.62 mg, 0.310 mmol) in DCM (2 mL) was added Pyridine (18.43 mg, 0.232 mmol) at room temperature. The resulting mixture was stirred at room temperature for 4 h. The resulting mixture was concentrated under reduced pressure. The residue was purified by silica gel column chromatography, eluted with PE/EtOAc (1:3) to afford {4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methyl 4-nitrophenyl carbonate (60 mg) as a white solid. LC-MS (ESI):809.5 [M+H]⁺.

### Synthesis of 2-{[({4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methoxy)carbonyl](methyl)amino}ethyl(1R,3R,15E,28R,2 9R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

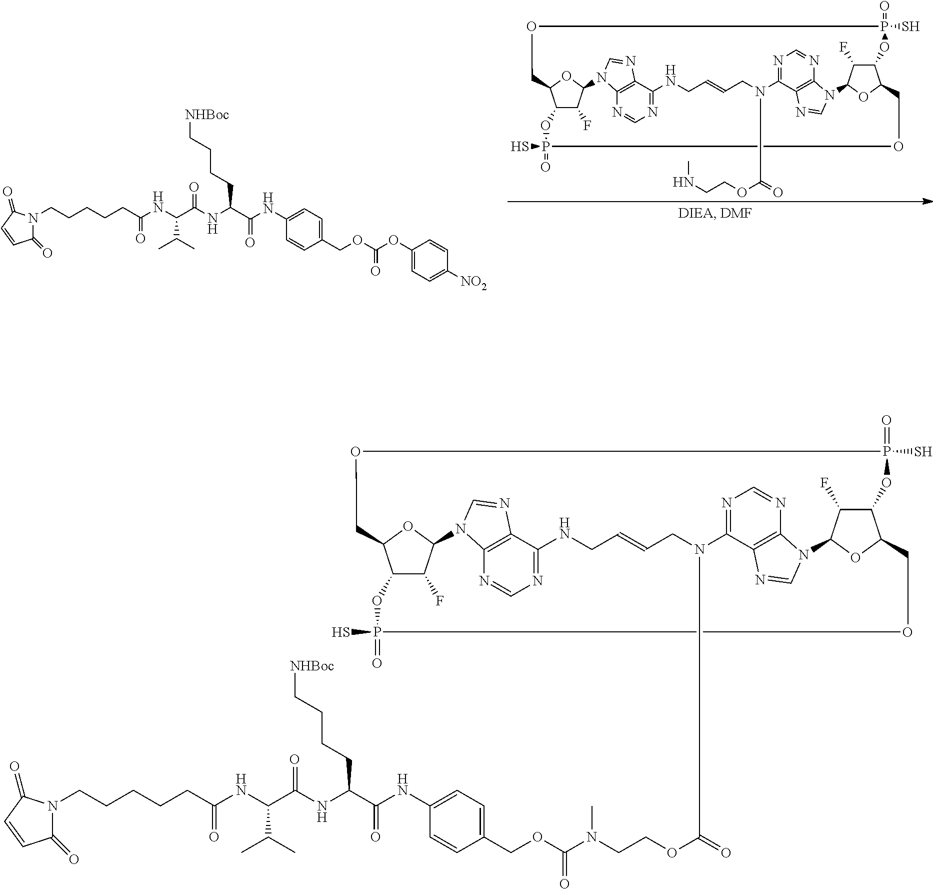 [SMILES: CC(C)[C@H](NC(=O)CCCCCN1C(=O)C=CC1=O)C(=O)N[C@@H](CCCCNC(=O)OC(C)(C)C)C(=O)Nc1ccc(COC(=O)Oc2ccc([N+](=O)[O-])cc2)cc1 ; CNCCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32 ; CC(C)[C@H](NC(=O)CCCCCN1C(=O)C=CC1=O)C(=O)N[C@@H](CCCCNC(=O)OC(C)(C)C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of {4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methyl 4-nitrophenyl carbonate (60 mg, 0.074 mmol) and 2-(methylamino)ethyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (62.88 mg, 0.074 mmol) in DMF (2 mL) was added DIEA (47.94 mg, 0.370 mmol) at room temperature. The resulting mixture was stirred at room temperature for 4 h. The resulting mixture was concentrated under reduced pressure. The residue was purified by Prep-TLC (DCM/MeOH 5:1) to afford 2-{[({4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (30 mg) as a white solid. LC-MS (ESI):1517.5 [M+H]⁺.

### Synthesis of 2-{[({4-[(2S)-6-amino-2-[(2S)-2-[6-(2,5-dioxo-2,5-dihydro-1H-pyrrol-1-yl)hexanamido]-3-methylbutanamido]hexanamido]phenyl}methoxy)carbonyl](methyl)amino}ethyl(1R,3R,15E,28R,2 9R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP15)

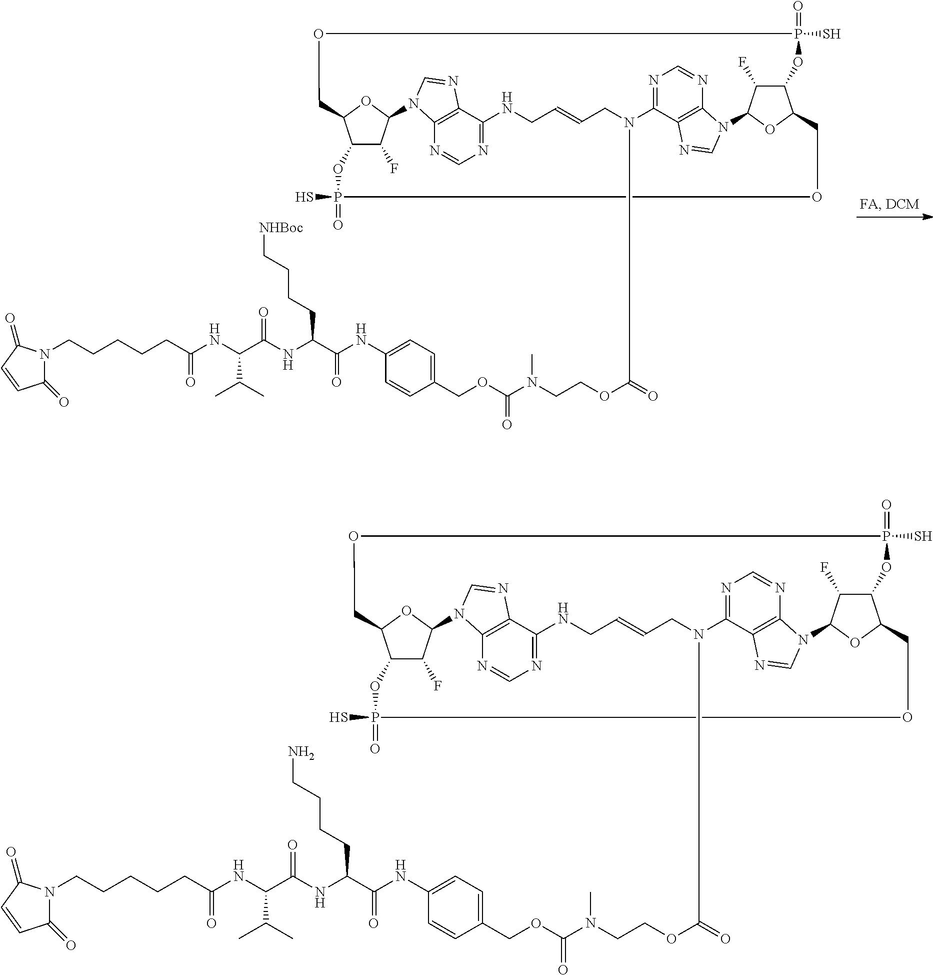 [SMILES: CC(C)[C@H](NC(=O)CCCCCN1C(=O)C=CC1=O)C(=O)N[C@@H](CCCCNC(=O)OC(C)(C)C)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](NC(=O)CCCCCN1C(=O)C=CC1=O)C(=O)N[C@@H](CCCCN)C(=O)Nc1ccc(COC(=O)N(C)CCOC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of 2-{[({4-[(2S)-6-{[(tert-butoxy)carbonyl]amino}-2-[(2S)-2-[6-(2,5-dioxo-2,5-dihydro-1H-pyrrol-1-yl)hexanamido]-3-methylbutanamido]hexanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg, 0.026 mmol) in DCM (2 mL) was added formic acid (2 mL) at room temperature. The resulting mixture was stirred at room temperature for 1 h. The resulting mixture was concentrated under reduced pressure. The crude product was purified by Prep-HPLC with the following conditions Column: XBridge Prep OBD C18 Column, 19*250 mm, 5 μm; Mobile Phase A: Water(50 mmol/L HCO₂NH₄), Mobile Phase B: MeCN; Flow rate: 25 mL/min; Gradient: 20% B to 30% B in 10 min, 30% B; Wavelength: 254 nm to afford 2-{[({4-[(2S)-6-amino-2-[(2S)-2-[6-(2,5-dioxo-2,5-dihydro-1H-pyrrol-1-yl)hexanamido]-3-methylbutanamido]hexanamido]phenyl}methoxy)carbonyl](methyl)amino}ethyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP15) (10.6 mg) as a white solid.

LC-MS (ESI): 1418.65 [M+H]⁺.

¹H NMR (400 MHz Methanol-d₄) δ 8.94-8.43 (m, 3H), 8.28-7.85 (m, 2H), 7.70-7.39 (m, 2H), 7.32-7.00 (m, 2H), 6.81 (m, 2H), 6.30 (s, 1H), 6.05-5.75 (m, 4H), 5.70-5.27 (m, 1H), 4.69-4.54 (m, 5H), 4.51-4.39 (m, 7H), 4.19-4.05 (m, 5H), 4.09-3.88 (m, 2H), 3.66-3.40 (m, 2H), 3.05-2.93 (m, 3H), 2.91-2.78 (m, 2H), 2.70 (s, 1H), 2.40-2.20 (m, 2H), 2.20-2.02 (m, 1H), 2.01-1.90 (m, 1H), 1.82-1.69 (m, 3H), 1.70-1.45 (m, 7H), 1.44-1.22 (m, 5H), 1.10-0.86 (m, 6H).

### 16. Synthesis of LP16

The synthesis of LP16 is shown below:

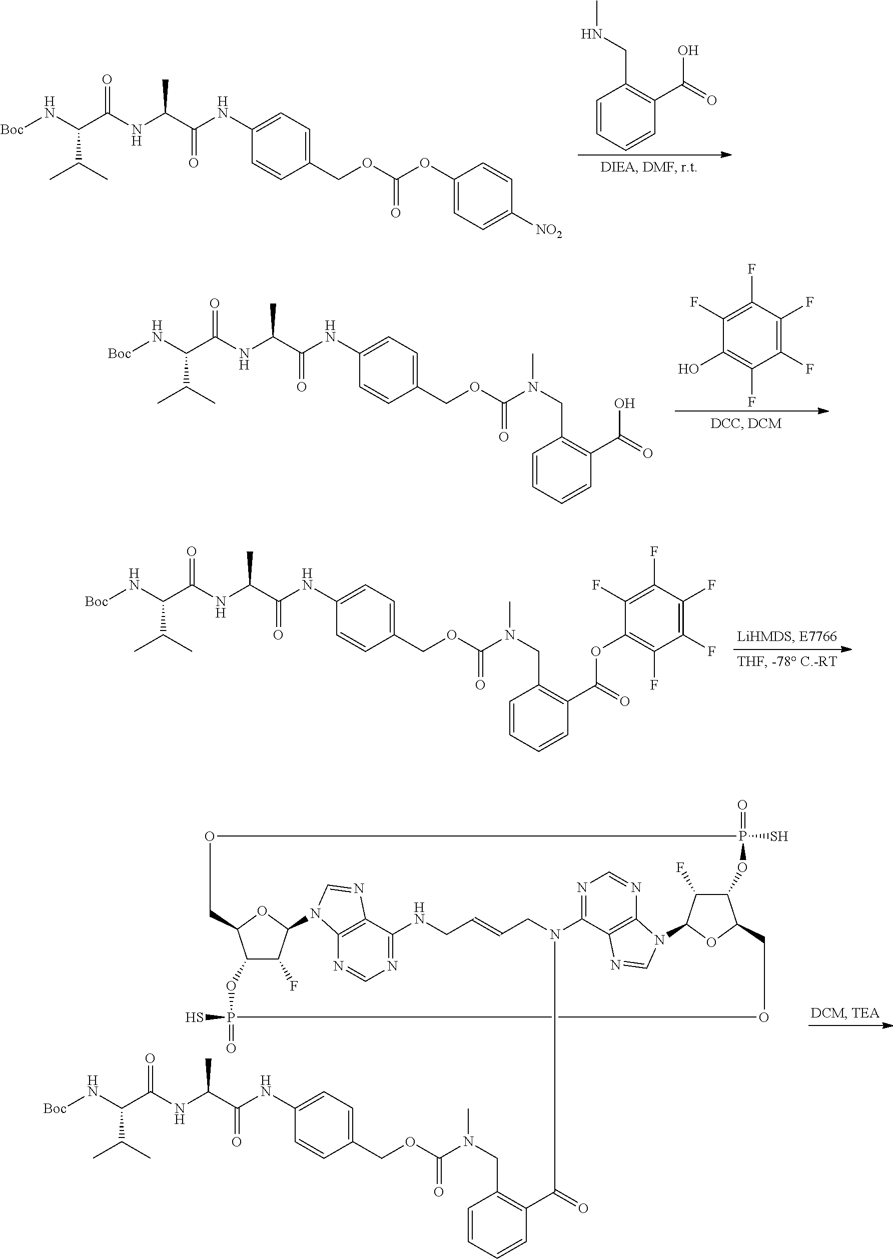

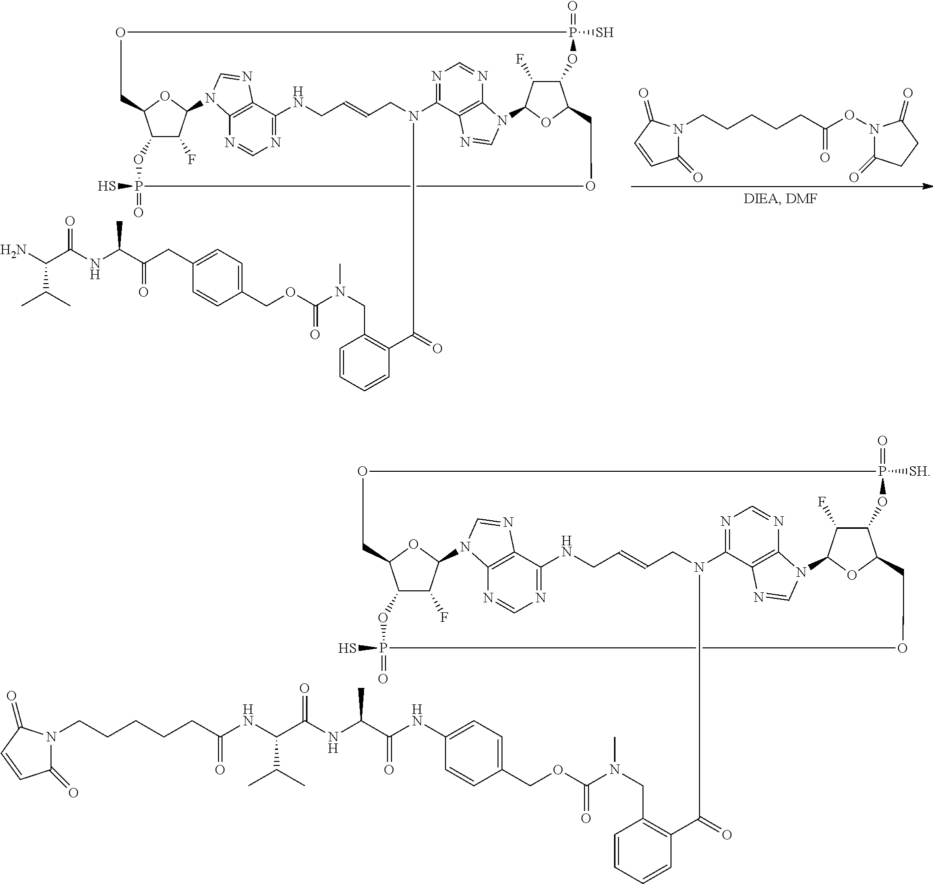 [SMILES: CC(C)[C@H](N)C(=O)N[C@@H](C)C(=O)Cc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoic acid

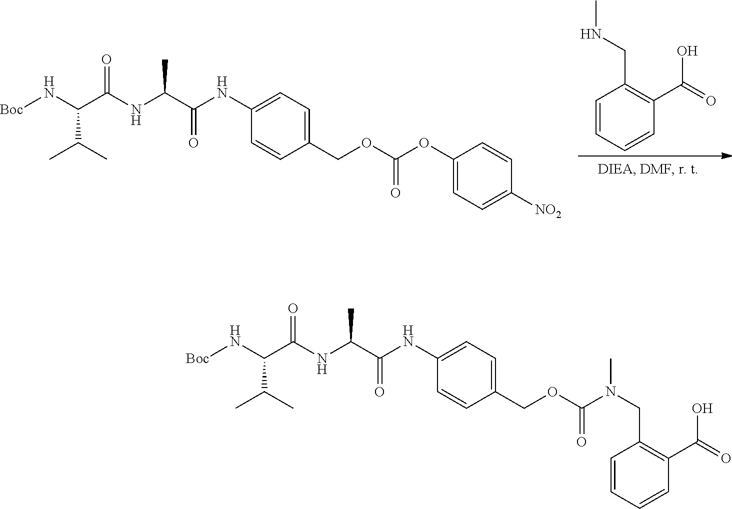 [SMILES: CC(C)[C@H](NC(=O)OC(C)(C)C)C(=O)N[C@@H](C)C(=O)Nc1ccc(COC(=O)Oc2ccc([N+](=O)[O-])cc2)cc1 ; CNCc1ccccc1C(=O)O ; CC(C)[C@H](NC(=O)OC(C)(C)C)C(=O)N[C@@H](C)C(=O)Nc1ccc(COC(=O)N(C)Cc2ccccc2C(=O)O)cc1]

2-[(Methylamino)methyl]benzoic acid (177 mg, 1.074 mmol) and DIEA (555 mg, 4.296 mmol) were added to a mixture of {4-[(2S)-2-[(2S)-2-[(tert-butoxycarbonyl)amino]-3-methylbutanamido]propanamido]phenyl}methyl 4-nitrophenyl carbonate (600 mg, 1.074 mmol) in DMF (5 mL). The resulting mixture was stirred at 25° C. for 2 h. The resulting mixture was diluted with EtOAc, washed with water, and concentrated. The residue was purified by silica gel column chromatography, eluted with CH₂Cl₂/MeOH (10:1). This resulted in 600 mg of 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoic acid as a white solid. LC-MS (ESI): 585 [M+H]⁺.

### Synthesis of 2,3,4,5,6-pentafluorophenyl 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoate

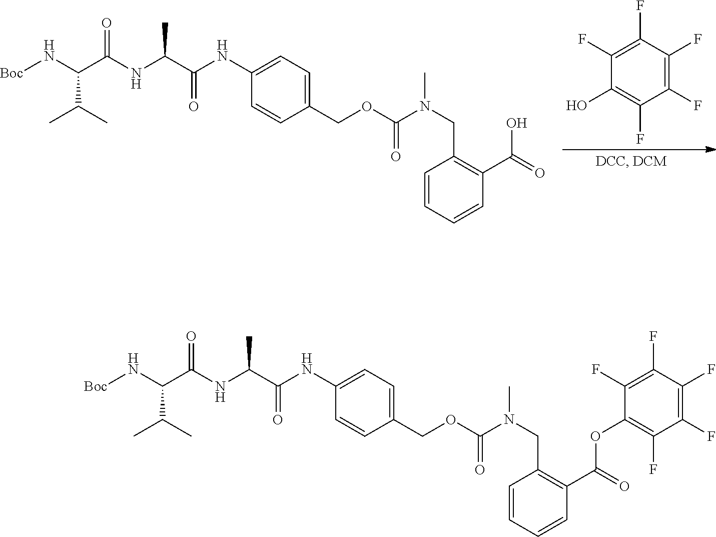 [SMILES: CC(C)[C@H](NC(=O)OC(C)(C)C)C(=O)N[C@@H](C)C(=O)Nc1ccc(COC(=O)N(C)Cc2ccccc2C(=O)O)cc1 ; Oc1c(F)c(F)c(F)c(F)c1F ; CC(C)[C@H](NC(=O)OC(C)(C)C)C(=O)N[C@@H](C)C(=O)Nc1ccc(COC(=O)N(C)Cc2ccccc2C(=O)Oc2c(F)c(F)c(F)c(F)c2F)cc1]

Pentafluorophenol (378 mg, 2.052 mmol) and DCC (424 mg, 2.052 mmol) were added to a mixture of 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoic acid (600 mg, 1.026 mmol) in DCM (10 mL). The resulting mixture was stirred at 25° C. for 1 h. The resulting mixture was concentrated under reduced pressure. The residue was purified by silica gel column chromatography, eluted with PE/EtOAc (1:1). This resulted in 830 mg of 2,3,4,5,6-pentafluorophenyl 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoate as a white solid. LC-MS (ESI): 751 [M+H]⁺.

### Synthesis of {4-[(2S)-2-[(2S)-2-{[(tert-butoxy)carbonyl]amino}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate

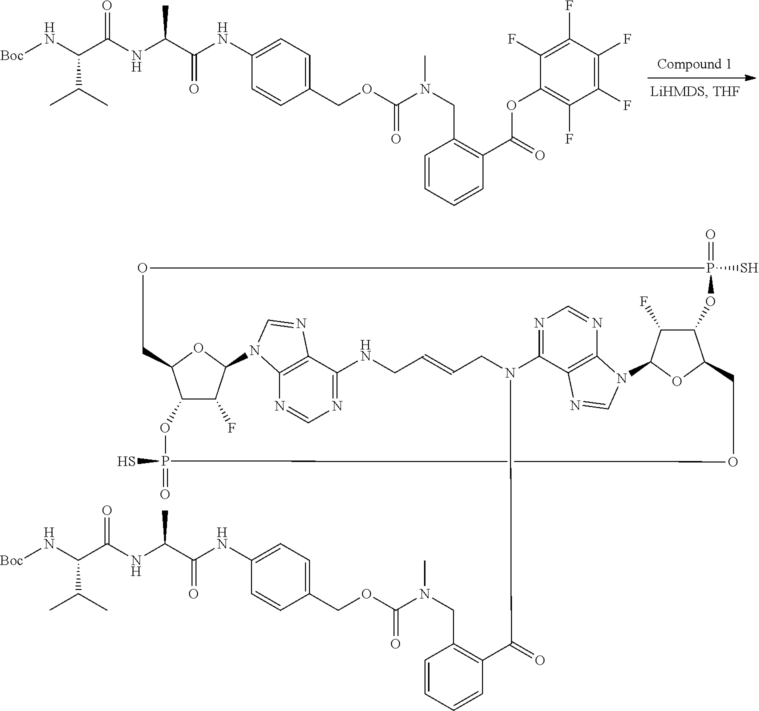 [SMILES: CC(C)[C@H](NC(=O)OC(C)(C)C)C(=O)N[C@@H](C)C(=O)Nc1ccc(COC(=O)N(C)Cc2ccccc2C(=O)Oc2c(F)c(F)c(F)c(F)c2F)cc1 ; CC(C)[C@H](NC(=O)OC(C)(C)C)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

LiHMDS (0.48 mmol) was added to a mixture of Compound 1 (60 mg, 0.080 mmol) in THF (5 mL). The resulting mixture was stirred at −78° C. for 30 min. To the above mixture was added 2,3,4,5,6-pentafluorophenyl 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoate (60 mg, 0.08 mmol) dropwise at −78° C. The resulting mixture was stirred for additional 3 h at −78° C. If Compound 1 was not consumed, 2 eq of LHMDS and 1 eq of 2,3,4,5,6-pentafluorophenyl 2-({[({4-[(2S)-2-[(2S)-2-{[(tert-butoxy)carbonyl]amino}-3-methylbutanamido]propanamido]phenyl}methoxy)carbonyl](methyl)amino}methyl)benzoate was added at 78° C. The reaction was quenched with AcOH. The resulting mixture was concentrated under reduced pressure. The residue was purified by silica gel column chromatography, eluted with DCM/MeOH (8:1). This resulted in 60 mg of {4-[(2S)-2-[(2S)-2-{[(tert-butoxy)carbonyl]amino}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate as a white solid. LC-MS (ESI): 1313 [M+H]⁺.

### Synthesis of {4-[(2S)-2-[(2S)-2-amino-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate

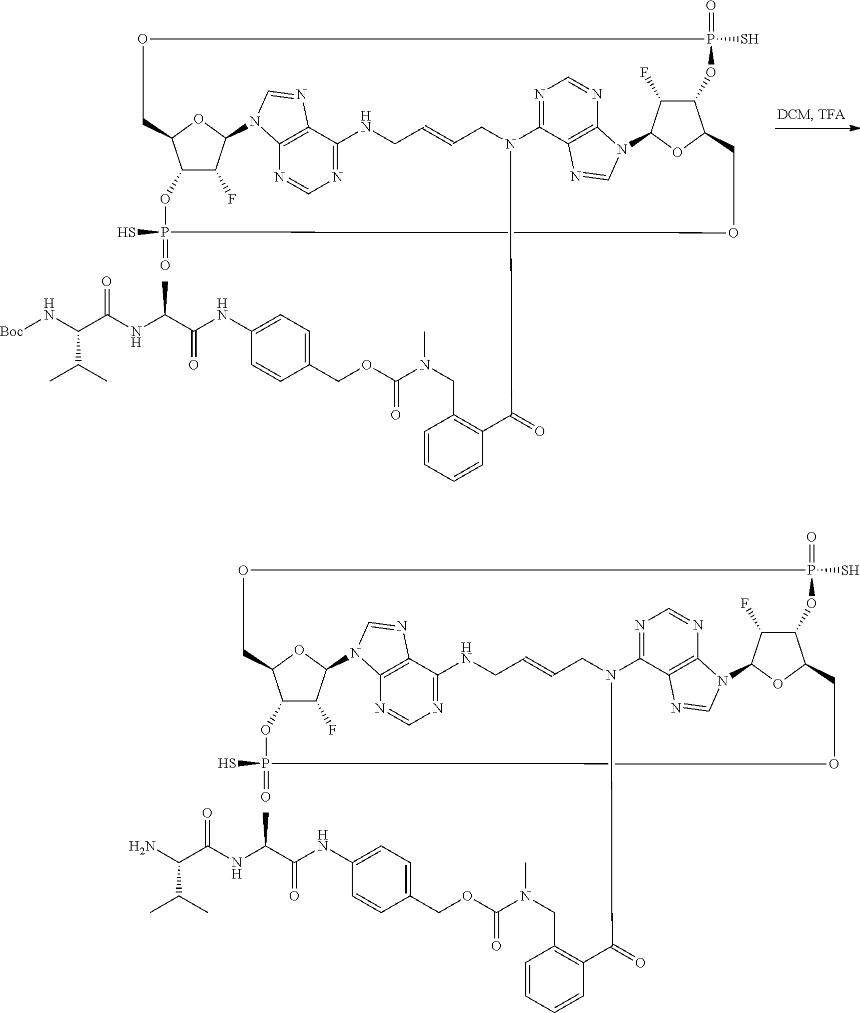 [SMILES: CC(C)[C@H](NC(=O)OC(C)(C)C)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

TFA (0.6 mL) was added to a mixture of {4-[(2S)-2-[(2S)-2-{[(tert-butoxy)carbonyl]amino}-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (80 mg, 0.061 mmol) in DCM (3.6 mL). The resulting mixture was stirred at 0° C. for 1 h. The resulting mixture was concentrated under reduced pressure. This resulted in 80 mg of {4-[(2S)-2-[(2S)-2-amino-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate as a crude oil. LC-MS (ESI): 1212 [M+H]⁺.

### Synthesis of {4-[(2S)-2-[(2S)-2-[6-(2,5-dioxo-2,5-dihydro-1H-pyrrol-1-yl)hexanamido]-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (LP16)

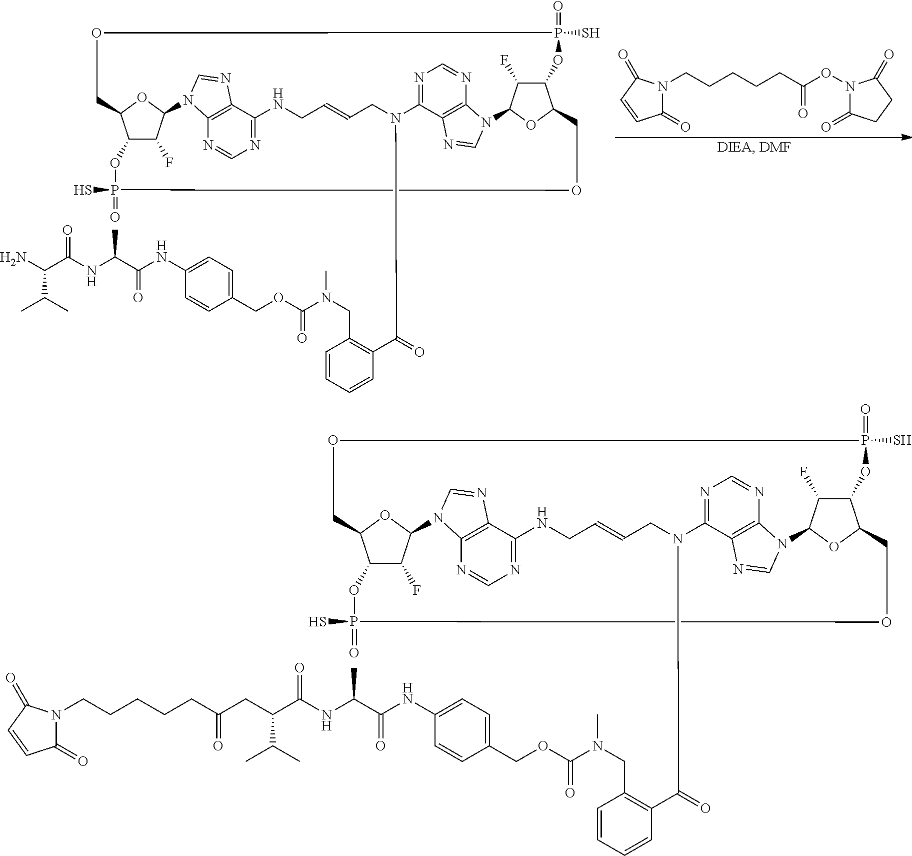

2,5-Dioxopyrrolidin-1-yl 6-(2,5-dioxopyrrol-1-yl)hexanoate (41 mg, 0.132 mmol) and DIEA (85 mg, 0.660 mmol) were added to a mixture of {4-[(2S)-2-[(2S)-2-amino-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (80 mg, 0.066 mmol) in DMF (2 mL). The resulting mixture was stirred at 25° C. for 1 h. The resulting mixture was concentrated under reduced pressure. The residue was purified by Prep-TLC (DCM/MeOH 7:1) and Prep-HPLC (Column: XBridge Prep Phenyl OBD Column, 19×250 mm, 5 μm; Mobile Phase A: Water (50 mmol HCO₂NH₄), Mobile Phase B: MeCN; Flow rate: 25 mL/min; Gradient: 25% B to 50% B in 15 min; Wavelength: 254 nm. This resulted in 25.3 mg of {4-[(2S)-2-[(2S)-2-[6-(2,5-dioxo-2,5-dihydro-1H-pyrrol-1-yl)hexanamido]-3-methylbutanamido]propanamido]phenyl}methyl N-({2-[(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (LP16) as a white solid.

LC-MS (ESI): 1406.55[M+H]⁺.

¹H NMR (400 MHz, Methanol-d₄) δ 8.97 (s, 1H), 8.52-8.28 (m, 2H), 8.26-8.13 (m, 2H), 8.09-7.99 (m, 1H), 7.64-7.50 (m, 2H), 7.41-7.33 (m, 1H), 7.28-7.17 (m, 2H), 7.06-6.93 (m, 2H), 6.80 (s, 2H), 6.43-6.23 (m, 2H), 6.13-5.45 (m, 4H), 5.14 (d, J=7.0 Hz, 3H), 4.87-4.72 (m, 3H), 4.70-4.30 (m, 8H), 4.24-4.13 (m, 1H), 4.10-4.00 (m, 1H), 3.99-3.83 (m, 1H), 3.77-3.59 (m, 1H), 3.54-3.42 (m, 3H), 3.31-3.14 (m, 3H), 3.02-2.91 (m, 3H), 2.37-2.25 (m, 2H), 2.16-2.04 (m, 1H), 1.68-1.53 (m, 4H), 1.47-1.42 (m, 3H), 1.40-1.25 (m, 3H), 1.03-0.95 (m, 6H).

### 17. Synthesis of LP17

The synthesis of LP17 is shown below:

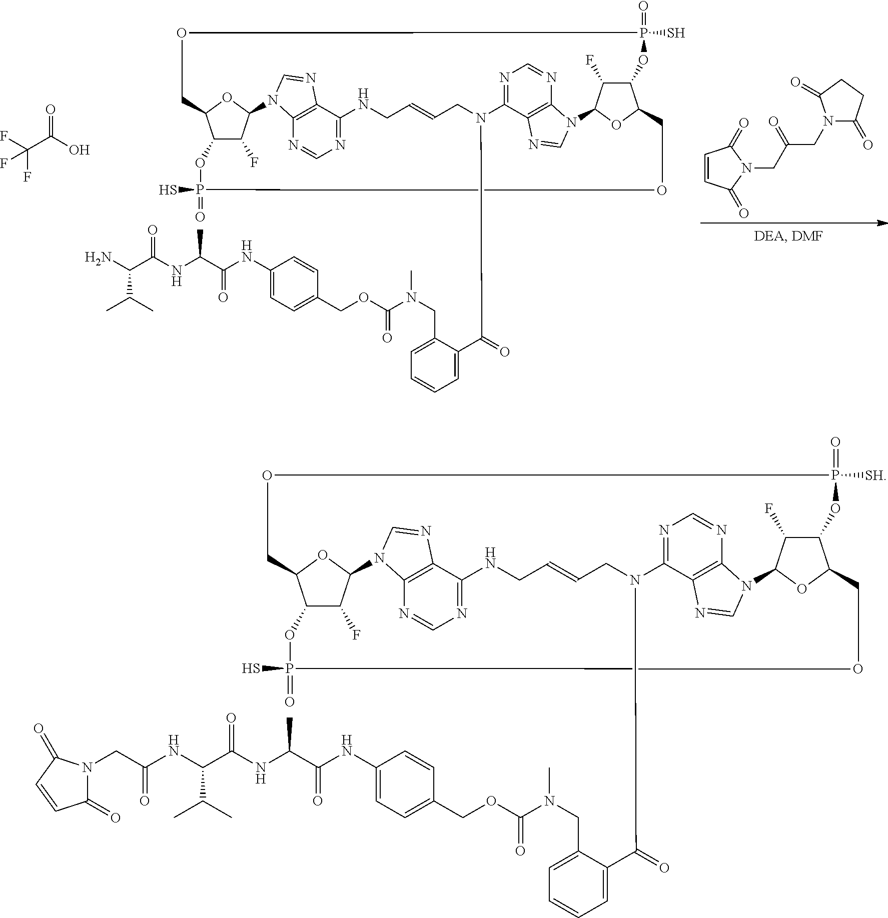 [SMILES: O=C(O)C(F)(F)F ; CC(C)[C@H](N)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CN1C(=O)C=CC1=O)CN1C(=O)CCC1=O ; CC(C)[C@H](NC(=O)CN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a solution of {4-[(25)-2-[(25)-2-amino-3-methylbutanamido]propanamido]phenyl}methyl N-({2-[(1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate trifluoroacetic acid salt (16 mg, 0.012 mmol) in DMF (1 mL) at room temperature was added DIEA (10.53 μl, 0.06 mmol) and 2,5-dioxopyrrolidin-1-yl 2-(2,5-dioxo-2,5-dihydro-1H-pyrrol-1-yl)acetate (4.56 mg, 0.018 mmol). The reaction mixture was stirred at room temperature for 2 hours. Reaction mixture was purified by reverse phase flash chromatography using 3% to 70% gradient of MeCN in water (0.1% formic acid) to give {4-[(25)-2-[(25)-2-[2-(2,5-dioxo-2,5-dihydro-1H-pyrrol-1-yl)acetamido]-3-methylbutanamido]propanamido]phenyl}methyl N-({2-[(1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carbonyl]phenyl}methyl)-N-methylcarbamate (LP17) (7.2 mg) as a colorless solid.

LC-MS (ESI): 1350.40 [M+H]⁺.

### 18. Synthesis of LP20

The synthesis of LP20 is shown below:

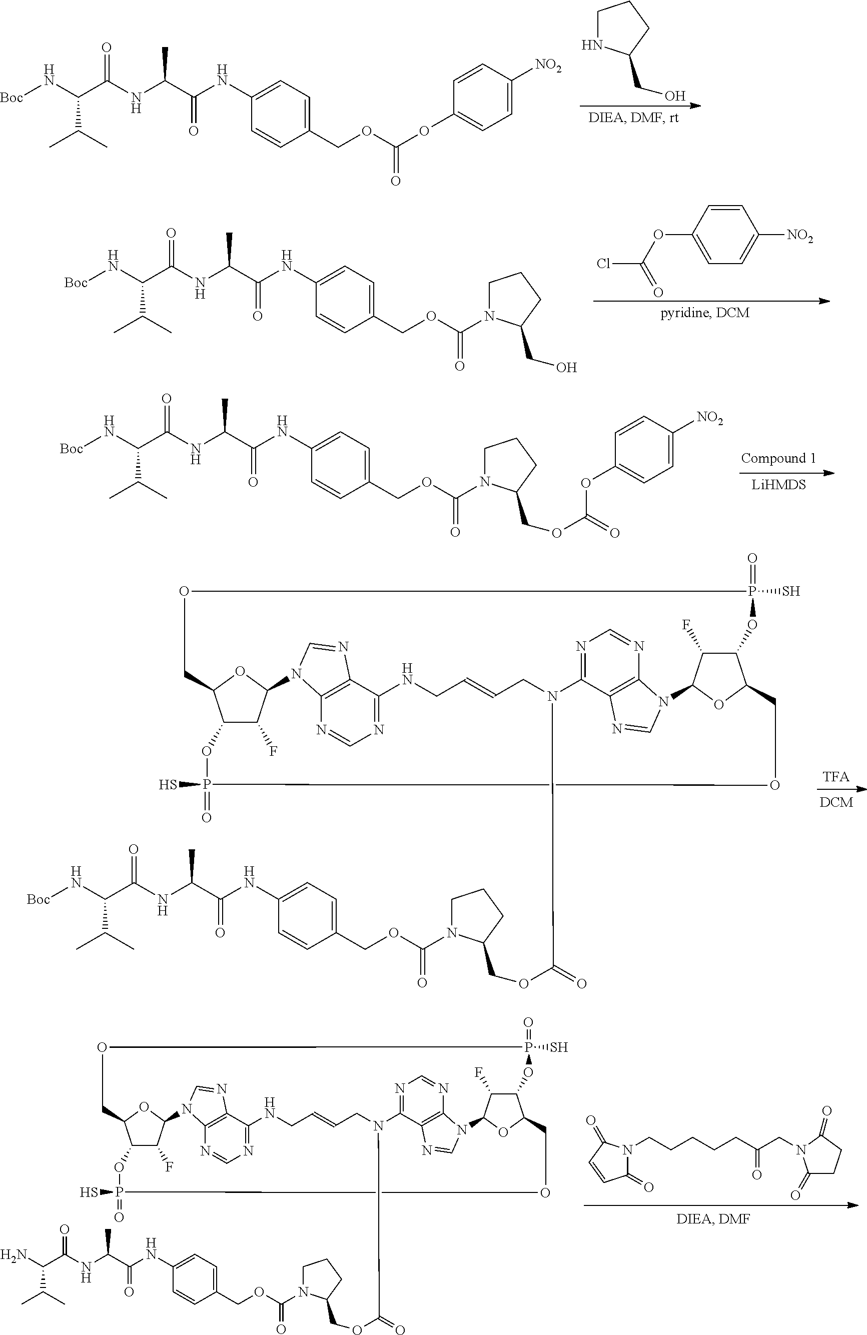

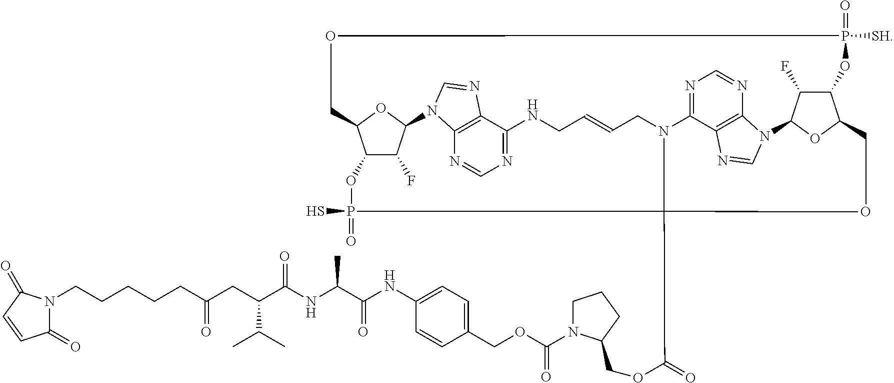 [SMILES: CC(C)[C@H](CC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of {4-[(2S)-2-[(2S)-2-{[(tert-butoxy)carbonyl]amino}-3-methylbutanamido]propanamido]phenyl}methyl (2S)-2-(hydroxymethyl)pyrrolidine-1-carboxylate

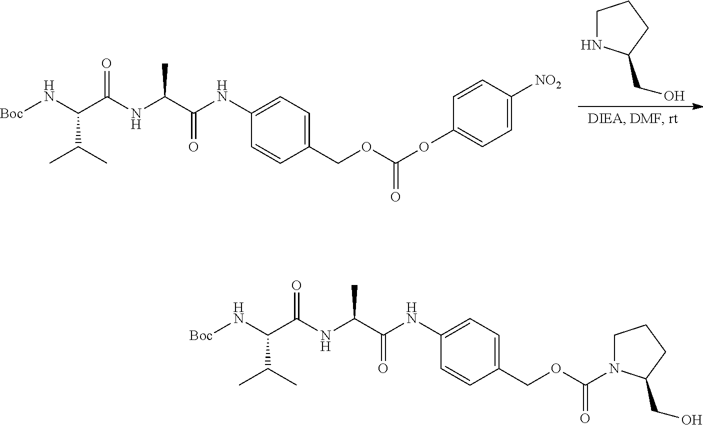 [SMILES: CC(C)[C@H](NC(=O)OC(C)(C)C)C(=O)N[C@@H](C)C(=O)Nc1ccc(COC(=O)Oc2ccc([N+](=O)[O-])cc2)cc1 ; OC[C@@H]1CCCN1 ; CC(C)[C@H](NC(=O)OC(C)(C)C)C(=O)N[C@@H](C)C(=O)Nc1ccc(COC(=O)N2CCC[C@H]2CO)cc1]

To a mixture of prolinol (50 mg, 0.494 mmol) in DMF (5 mL) were added {4-[(25)-2-[(25)-2-{[(tert-butoxy)carbonyl]amino}-3-methylbutanamido]propanamido]phenyl}methyl 4-nitrophenyl carbonate (276.12 mg, 0.494 mmol) and DIEA (191.67 mg, 1.482 mmol) at room temperature. The resulting mixture was stirred at room temperature overnight. The resulting mixture was concentrated under reduced pressure. The residue was purified by silica gel column chromatography, eluted with CH₂Cl₂/MeOH (10:1) to afford {4-[(25)-2-[(25)-2-{[(tert-butoxy)carbonyl]amino}-3-methylbutanamido]propanamido]phenyl}methyl (2)-2-(hydroxymethyl)pyrrolidine-1-carboxylate (250 mg) as a white oil. LC-MS (ESI): 521.6 [M+H]⁺.

### Synthesis of {4-[(2S)-2-[(2S)-2-{[(tert-butoxy)carbonyl]amino}-3-methylbutanamido]propanamido]phenyl}methyl (2S)-2-({[(4-nitrophenoxy)carbonyl]oxy}methyl)pyrrolidine-1-carboxylate

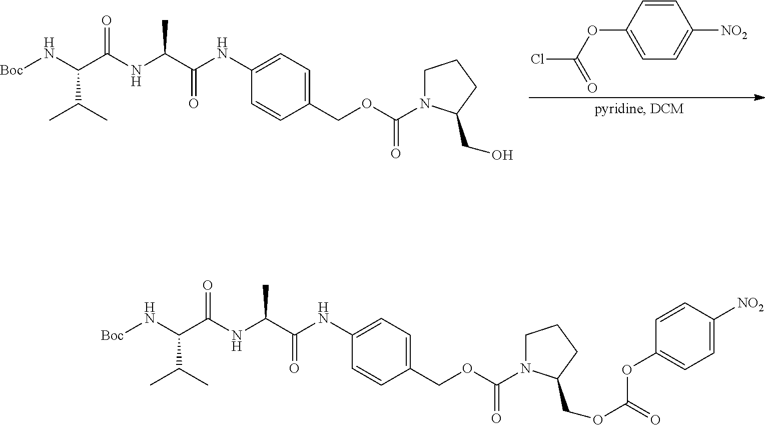 [SMILES: CC(C)[C@H](NC(=O)OC(C)(C)C)C(=O)N[C@@H](C)C(=O)Nc1ccc(COC(=O)N2CCC[C@H]2CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N2CCC[C@H]2COC(=O)Oc2ccc([N+](=O)[O-])cc2)cc1]

To a mixture of {4-[(2S)-2-[(2S)-2-{[(tert-butoxy)carbonyl]amino}-3-methylbutanamido]propanamido]phenyl}methyl (2S)-2-(hydroxymethyl)pyrrolidine-1-carboxylate (250 mg, 0.480 mmol) and 4-nitrophenyl carbonochloridate (145.18 mg, 0.720 mmol) in DCM (7 mL) was added Pyridine (56.97 mg, 0.720 mmol) at room temperature. The resulting mixture was stirred at RT overnight. The resulting mixture was concentrated under reduced pressure. The residue was purified by Prep-TLC (PE/EtOAc 1:3) to afford {4-[(2S)-2-[(2S)-2-{[(tert-butoxy)carbonyl]amino}-3-methylbutanamido]propanamido]phenyl}methyl (2S)-2-({[(4-nitrophenoxy)carbonyl]oxy}methyl)pyrrolidine-1-carboxylate (230 mg) as a white oil. LC-MS (ESI): 686.7 [M+H]⁺.

### Synthesis of [(2S)-1-[({4-[(2S)-2-[(2S)-2-{[(tert-butoxy)carbonyl]amino}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

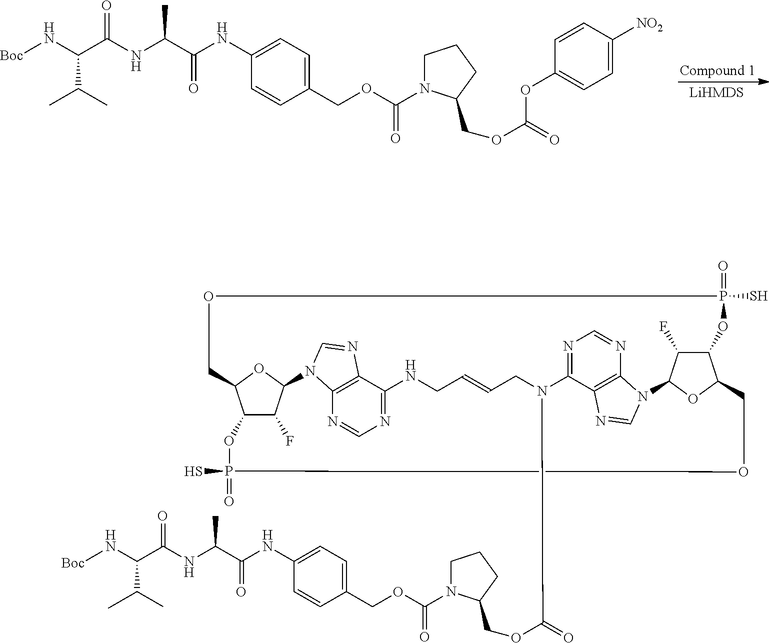 [SMILES: CC(C)[C@H](NC(=O)OC(C)(C)C)C(=O)N[C@@H](C)C(=O)Nc1ccc(COC(=O)N2CCC[C@H]2COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of Compound 1 (60 mg, 0.080 mmol) in THF (5 mL) were added LiHMDS (0.480 mmol) at −78° C. under nitrogen atmosphere. The resulting mixture was stirred at −78° C. for 30 min. To the resulting mixture was added {4-[(2S)-2-[(2S)-2-{[(tert-butoxy)carbonyl]amino}-3-methylbutanamido]propanamido]phenyl}methyl (2S)-2-({[(4-nitrophenoxy)carbonyl]oxy}methyl)pyrrolidine-1-carboxylate (55.11 mg, 0.080 mmol) at −78° C. under nitrogen atmosphere. The resulting mixture was slowly warmed to 0° C. over an hour. The reaction was quenched by the addition of AcOH. The resulting mixture was concentrated under reduced pressure. The residue was purified by Prep-TLC (EtOAc/MeOH 3:1) to afford [(2S)-1-[({4-[(2S)-2-[(2S)-2-{[(tert-butoxy)carbonyl]amino}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (50 mg) as a white solid. LC-MS (ESI): 1293.4 [M+H]⁺.

### Synthesis of [(2S)-1-[({4-[(2S)-2-[(2S)-2-amino-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

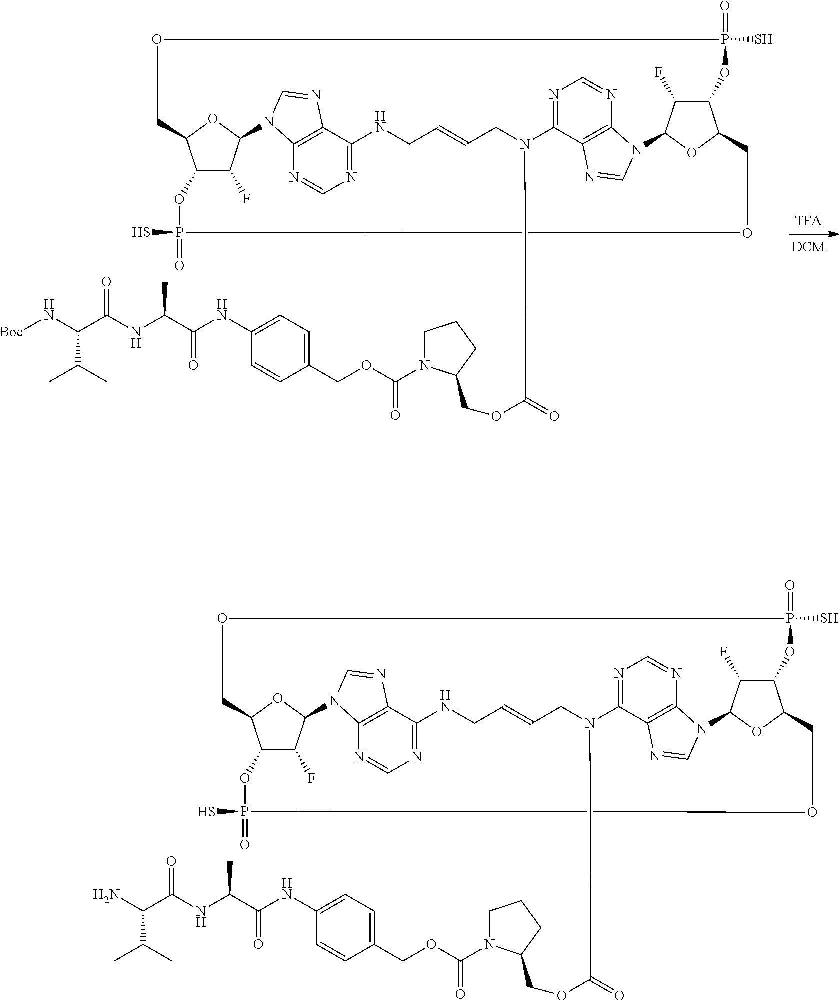 [SMILES: CC(C)[C@H](NC(=O)OC(C)(C)C)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of [(2S)-1-[({4-[(2S)-2-[(2S)-2-{[(tert-butoxy)carbonyl]amino}-3 methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (50 mg, 0.039 mmol) in DCM (6 mL) was added TFA (1 mL) at 0° C. The resulting mixture was stirred at 0° C. for 1 h. The resulting mixture was concentrated under reduced pressure. The crude was used in the next step directly without further purification. LC-MS (ESI): 1193.1 [M+H]⁺.

### Synthesis of [(2S)-1-[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP20)

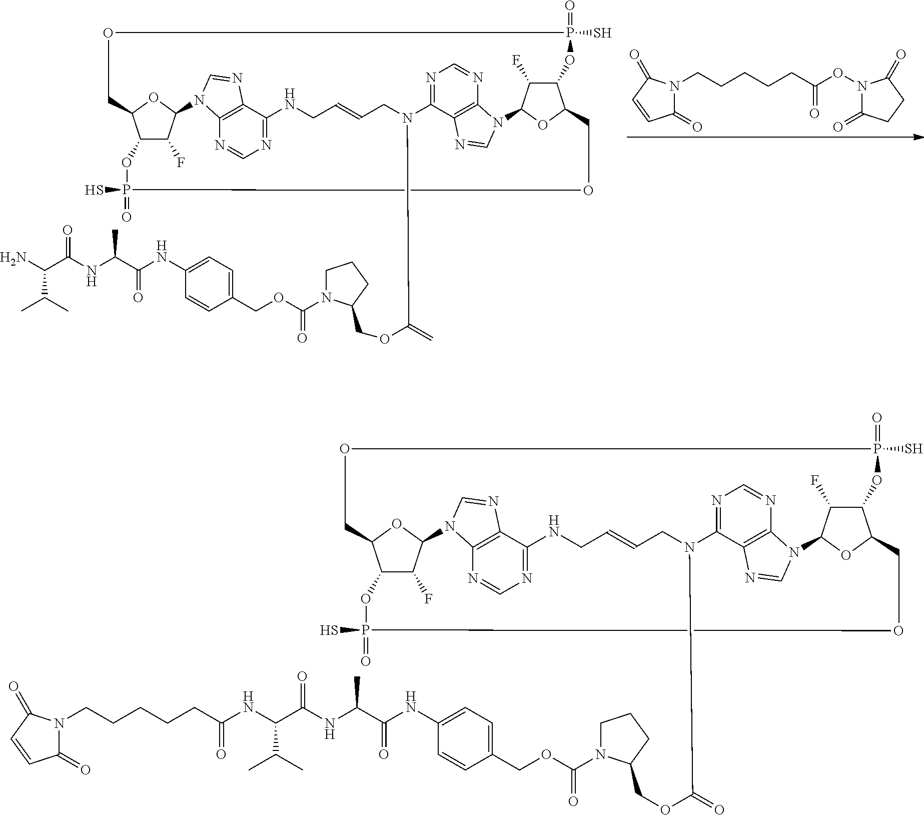

To a mixture of [(2S)-1-[({4-[(2S)-2-[(2S)-2-amino-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (10 mg, 0.008 mmol) and 2,5-dioxopyrrolidin-1-yl 6-(2,5-dioxopyrrol-1-yl)hexanoate (25.84 mg, 0.084 mmol) in DMF(3 mL) was added DIEA (16.25 mg, 0.126 mmol) at room temperature. The resulting mixture was stirred at room temperature for 1 h. The resulting mixture was concentrated under vacuum. The residue was purified by Prep-TLC(DCM/MeOH 5:1) and Prep-HPLC with the following conditions Column: XBridge Prep OBD C18 Column, 19*250 mm, 5 μm; Mobile Phase A: Water(50 mmol/L HCO₂NH₄), Mobile Phase B: MeCN; Flow rate: 25 mL/min; Gradient: 25% B to 35% B in 12 min, 35% B; Wavelength: 254 nm to afford [(2S)-1-[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP20) (11.4 mg) as a white solid. LC-MS (ESI):1386.40 [M+H]⁺; ¹H NMR (400 MHz, Methanol-d₄) δ 9.14-8.62 (m, 3H), 8.24-7.98 (m, 1H), 7.69-7.47 (m, 2H), 7.41-7.17 (m, 2H), 6.94-6.64 (m, 2H), 6.62-6.14 (m, 2H), 6.11-5.31 (m, 4H), 5.09-4.92 (m, 3H), 4.74-4.58 (m, 4H), 4.54-4.39 (m, 4H), 4.35-4.17 (m, 2H), 4.12-3.87 (m, 4H), 3.79-3.55 (m, 1H), 3.56-3.41 (m, 3H), 3.23-2.98 (m, 1H), 2.70 (s, 1H), 2.36-2.27 (m, 2H), 2.24-2.05 (m, 2H), 2.00-1.75 (m, 1H), 1.74-1.51 (m, 8H), 1.52-1.43 (m, 3H), 1.40-1.22 (m, 5H), 1.06-0.85 (m, 6H).

### 19. Synthesis of LP18

The synthesis of LP18 is shown below:

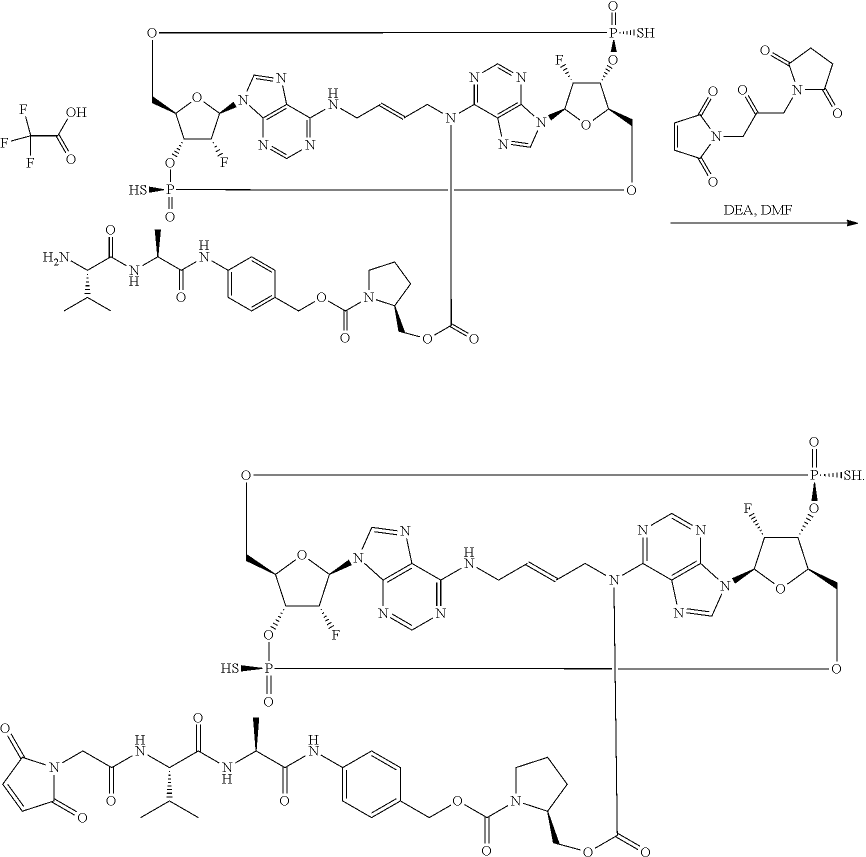 [SMILES: O=C(O)C(F)(F)F ; CC(C)[C@H](N)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CN1C(=O)C=CC1=O)CN1C(=O)CCC1=O ; CC(C)[C@H](NC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

Compound [(25)-1-[({4-[(25)-2-[(25)-2-[2-(2,5-dioxo-2,5-dihydro-1H-pyrrol-1-yl)acetamido]-3-methylbutanamido]propanamido]phenyl}methoxy)carbonyl]pyrrolidin-2-yl]methyl(1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP18) was obtained by a procedure analogous to the procedure for LP20 by using 2,5-dioxopyrrolidin-1-yl 2-(2,5-dioxo-2,5-dihydro-1H-pyrrol-1-yl)acetate instead of 2,5-dioxopyrrolidin-1-yl 6-(2,5-dioxopyrrol-1-yl)hexanoate.

LC-MS (ESI): 1330.72 [M+H]⁺.

### 20. Synthesis of LP19

The synthesis of LP19 is shown below:

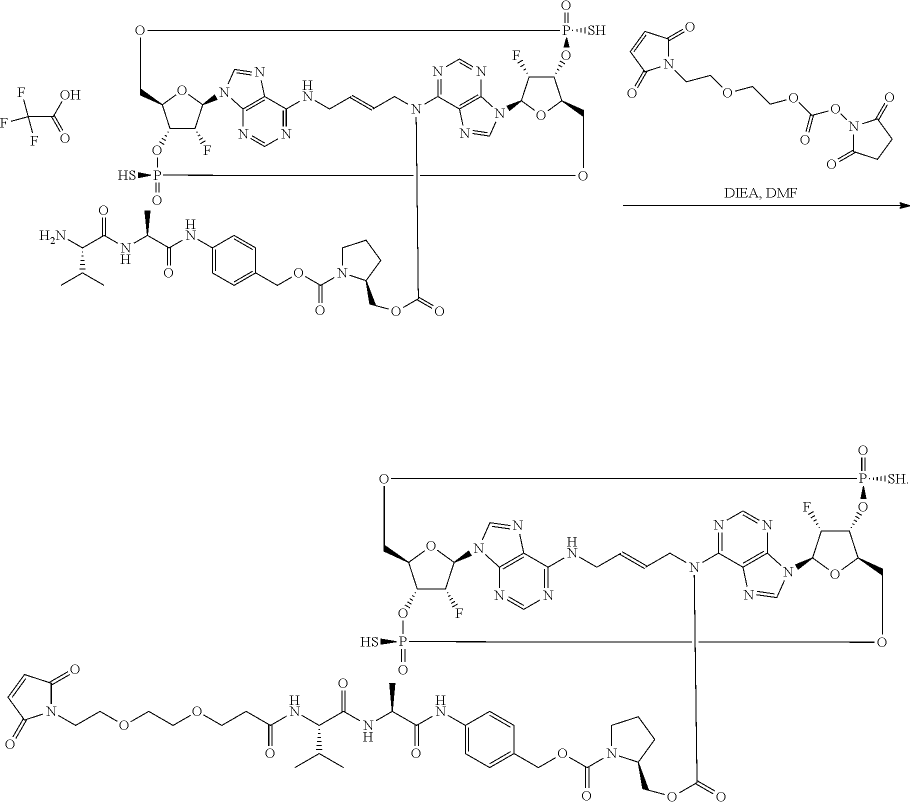 [SMILES: O=C(O)C(F)(F)F ; CC(C)[C@H](N)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(OCCOCCN1C(=O)C=CC1=O)ON1C(=O)CCC1=O ; CC(C)[C@H](NC(=O)CCOCCOCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

Compound [(2S)-1-[({4-[(2S)-2-[(2S)-2-(3-{2-[2-(2,5-dioxo-2,5-dihydro-1H-pyrrol-1-yl)ethoxy]ethoxy}propanamido)-3-methylbutanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP19) was obtained by a procedure analogous to the procedure for LP20 by using 2,5-dioxopyrrolidin-1-yl 3-{2-[2-(2,5-dioxo-2,5-dihydro-1H-pyrrol-1-yl)ethoxy]ethoxy}propanoate instead of 2,5-dioxopyrrolidin-1-yl 6-(2,5-dioxopyrrol-1-yl)hexanoate.

LC-MS (ESI): 1432.88 [M+H]⁺.

### 21. Synthesis of LP29

The synthesis of LP29 is shown below:

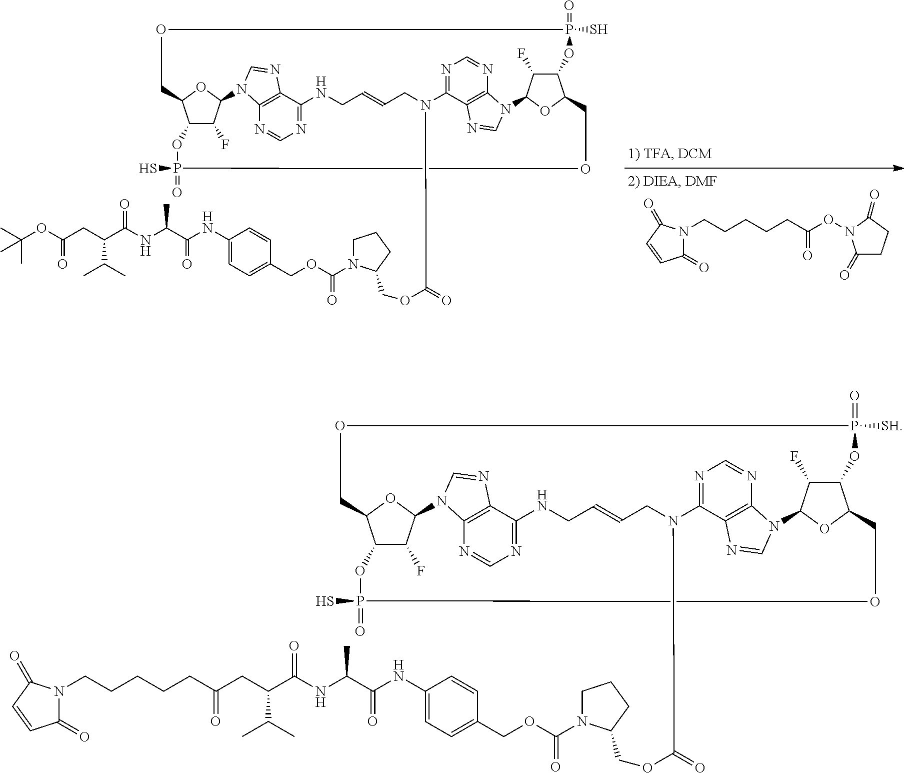 [SMILES: CC(C)[C@H](CC(=O)OC(C)(C)C)C(=O)N[C@@H](C)C(=O)Nc1ccc(COC(=O)N2CCC[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](CC(=O)CCCCCN1C(=O)C=CC1=O)C(=O)N[C@@H](C)C(=O)Nc1ccc(COC(=O)N2CCC[C@@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a mixture of [(2R)-1-[({4-[(2S)-2-[(2S)-2-{[(tert-butoxy)carbonyl]amino}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (20 mg, 0.015 mmol) in DCM (0.5 mL) was added TFA (0.5 mL). The resulting mixture was stirred at room temperature for 15 min. The resulting mixture was concentrated under reduced pressure and dissolved in DCM/toluene and then concentrated under reduce pressure three times. The crude residue was dissolved in DMF (0.5 mL) and then triethylamine (31.3 mg, 0.309 mmol) and 2,5-dioxopyrrolidin-1-yl 6-(2,5-dioxopyrrol-1-yl)hexanoate (9.5 mg, 0.031 mmol) were added into the mixture. The resulting mixture was stirred at room Temperature for 30 min. The resulting mixture was quenched with formic acid (20.2 mg, 0.387 mmol) and directly purified by ODS silica gel chromatography (0.1% formic acid in MeCN/water=1:9 to 4:6) to afford the crude product. The crude product was purified by ODS silica gel chromatography (0.1% TFA in MeCN/water=1:9 to 4:6) to afford [(2R)-1-[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP29) (3.97 mg).

LC-MS (ESI): 1386.59 [M+H]⁺.

### 22. Synthesis of LP25

The synthesis of LP25 is shown below:

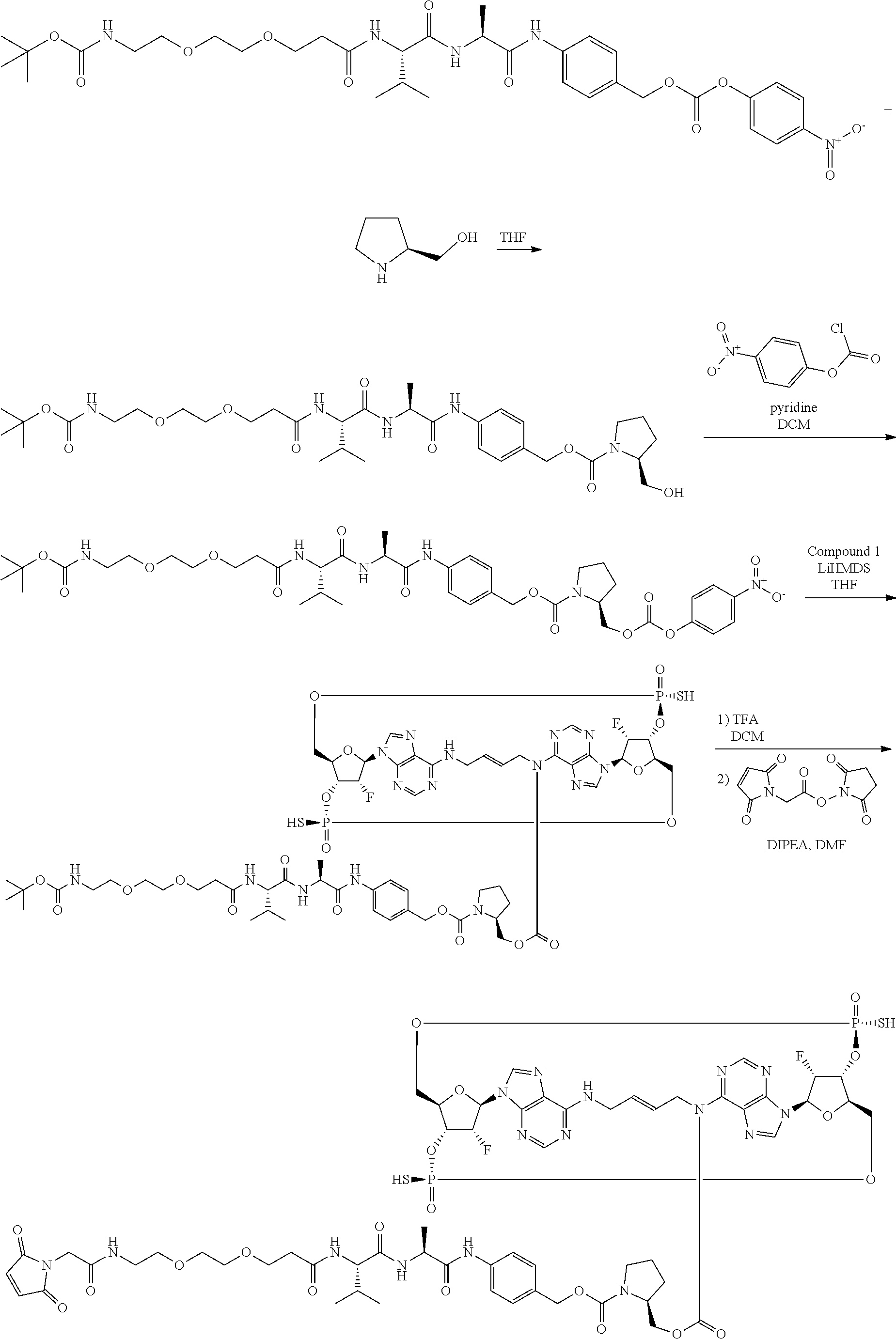

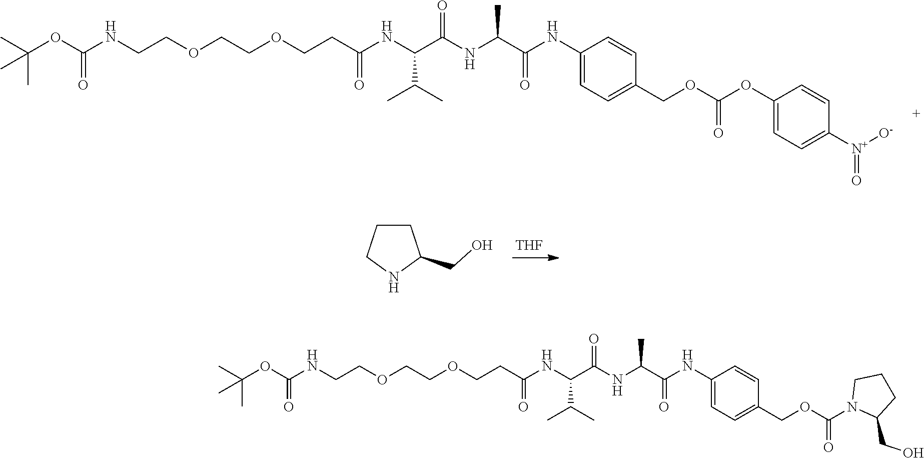 [SMILES: CC(C)[C@H](NC(=O)CCOCCOCCNC(=O)OC(C)(C)C)C(=O)N[C@@H](C)C(=O)Nc1ccc(COC(=O)Oc2ccc([N+](=O)[O-])cc2)cc1 ; OC[C@@H]1CCCN1 ; CC(C)[C@H](NC(=O)CCOCCOCCNC(=O)OC(C)(C)C)C(=O)N[C@@H](C)C(=O)Nc1ccc(COC(=O)N2CCC[C@H]2CO)cc1 ; "THF"]

To a solution of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl 4-nitrophenyl carbonate (100 mg, 0.139 mmol) in THF (2.0 mL) at room temperature was added L-prolinol (42.3 mg, 0.148 mmol) in THF (0.4 mL). The reaction mixture was stirred at room temperature for 2 h 50 min. Solvent was removed under reduced pressure. The residue was purified by silica gel column chromatography (EtOAc-MeOH=100:0 to 85:15) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-2-(hydroxymethyl)pyrrolidine-1-carboxylate (98.8 mg) as a colorless oil. LC-MS (ESI): 680.45 [M+]⁺.

### Synthesis of {4-[(2S)-2-[(2S)-2-(3-[2-(2-{[(tert-butoxy)carbonyl]amino}ethoxy)ethoxy]propanamido}-3-methylbutanamido]propanamido]phenyl}methyl (2S)-2-{[((4-nitrophenoxy)carbonyl]oxy}methyl)pyrrolidine-1-carboxylate

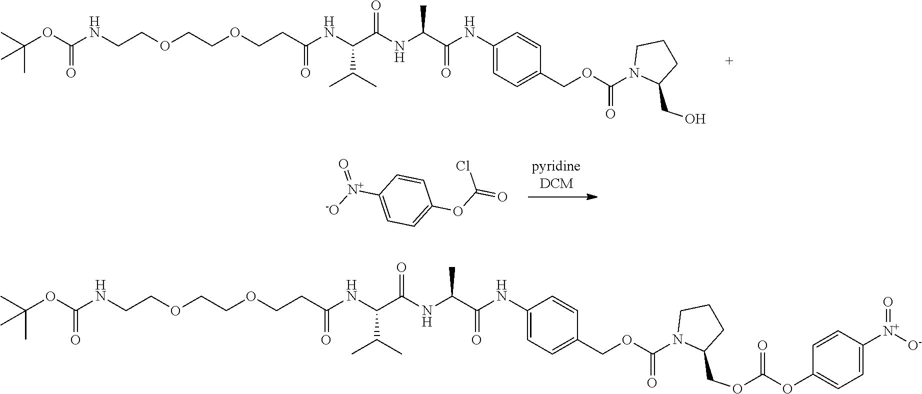

To a solution of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-2-(hydroxymethyl)pyrrolidine-1-carboxylate (98.8 mg, 0.139 mmol) in DCM (3.0 mL) at room temperature were added pyridine (22.4 μL, 0.277 mmol) and 4-nitrophenyl chloroformate (55.1 mg, 0.273 mmol). The mixture was stirred at room temperature for 2 h 45 min. Ethyl acetate (10 mL) and 10% aqueous citric acid (5 mL) were added to the reaction mixture, and organic layer was separated, washed with brine, dried over sodium sulfate, filtered, and concentrated under reduced pressure. The residue was purified by silica gel column chromatography (EtOAc-MeOH=100:0 to 90:10) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-2-({[(4-nitrophenoxy)carbonyl]oxy}methyl)pyrrolidine-1-carboxylate (91.9 mg) as a colorless oil. LC-MS (ESI): 845.54 [M+H]⁺.

### Synthesis of [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

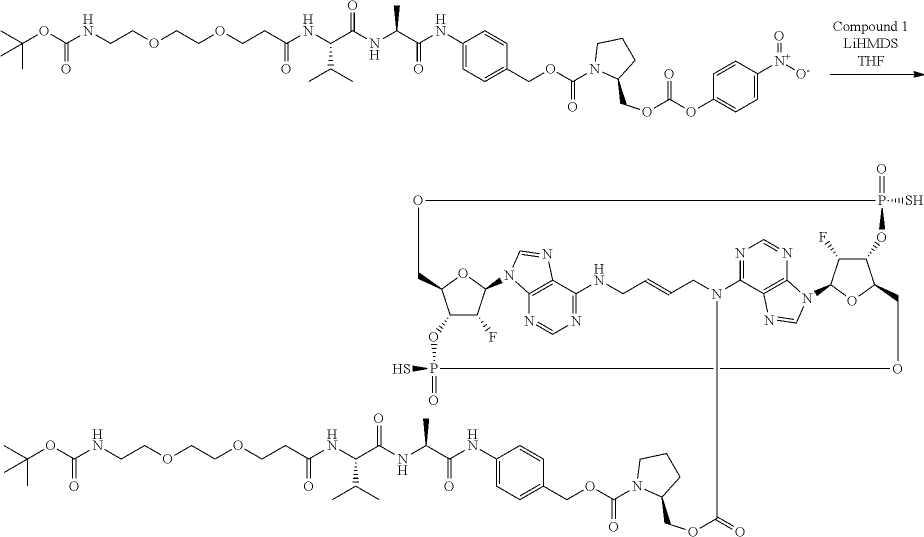 [SMILES: CC(C)[C@H](NC(=O)CCOCCOCCNC(=O)OC(C)(C)C)C(=O)N[C@@H](C)C(=O)Nc1ccc(COC(=O)N2CCC[C@H]2COC(=O)Oc2ccc([N+](=O)[O-])cc2)cc1 ; CC(C)[C@H](NC(=O)CCOCCOCCNC(=O)OC(C)(C)C)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a white suspension of Compound 1 (45 mg, 0.053 mmol) in THF (5.0 mL) at room temperature was added LiHMDS (0.487 mL, 0.633 mmol) dropwise for 4 min. The reaction mixture was stirred at rt for 30 min. Then it was cooled to −78° C. To the mixture was added {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-2-({[(4-nitrophenoxy)carbonyl]oxy}methyl)pyrrolidine-1-carboxylate (46.3 mg, 0.055 mmol) in THF (0.5 mL) with THF rinse (0.25 mL×2). The reaction mixture was stirred at −78° C. for 10 min. Then, the reaction mixture was allowed to warm to room temperature. After stirring at room temperature for 30 min, the reaction mixture was cooled to −78° C., and was quenched with acetic acid (0.076 mL, 1.319 mmol) at −78° C. Then it was stirred at 0° C. for 1 h 45 min. The mixture was concentrated under reduced pressure. The residue was purified by ODS silica gel column chromatography (H₂O-MeCN (0.1% formic acid)=95:5 to 35:65) to give [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (53.3 mg) as a white solid. LC-MS (ESI): 1452.48 [M+H]⁺.

### Synthesis of [(2S)-1-[({4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP25)

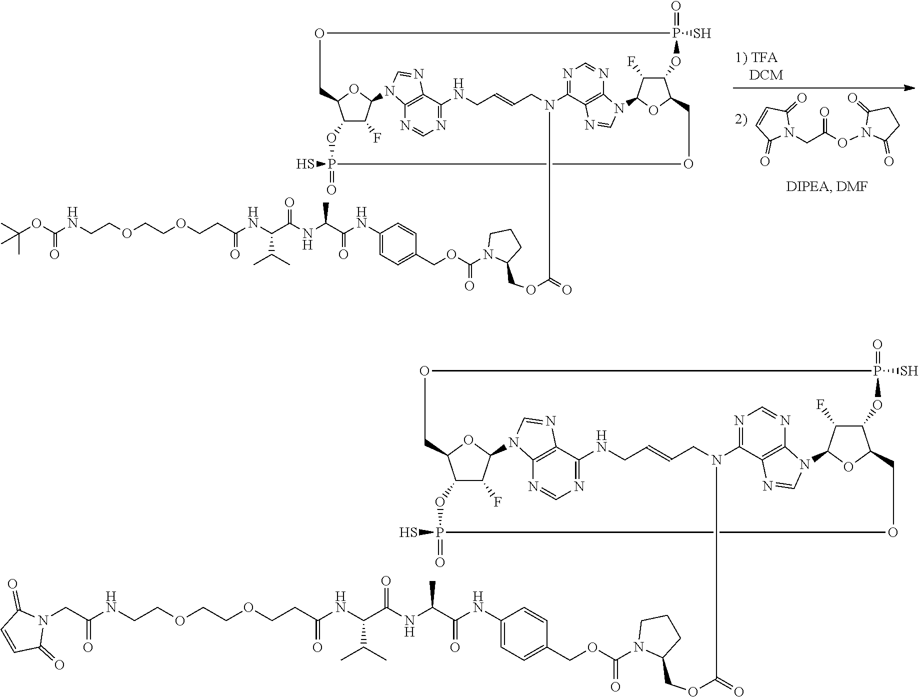 [SMILES: CC(C)[C@H](NC(=O)CCOCCOCCNC(=O)OC(C)(C)C)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CN1C(=O)C=CC1=O)ON1C(=O)CCC1=O ; CC(C)[C@H](NC(=O)CCOCCOCCNC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a suspension of [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (52.8 mg, 0.036 mmol) in dichloromethane (4.0 mL) at 0° C. was added trifluoroacetic acid (0.4 mL). The mixture was stirred at room temperature for 1 h. All volatiles were removed azeotropically with toluene (three times). The residue was dried under high vacuum. This material was dissolved in DMF (2.0 mL). To the solution were added DIEA (63 μL, 0.364 mmol) and 2,5-dioxopyrrolidin-1-yl 2-(2,5-dioxo-2,5-dihydro-1H-pyrrol-1-yl)acetate (36.7 mg, 0.145 mmol) at 0° C. The mixture was stirred at 0° C. for 1 h. The reaction was quenched by addition of formic acid (0.014 mL, 0.364 mmol) at 0° C. All volatiles were removed under high vacuum. The residue was purified by ODS silica gel column chromatography (H₂O-MeCN (0.1% formic acid)=95:5 to 55:45) to give [(2S)-1-[({4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP25) (31.56 mg) as a white solid.

LC-MS (ESI): 1489.43 [M+H]⁺.

### 23. Synthesis of LP28

The synthesis of LP28 is shown below:

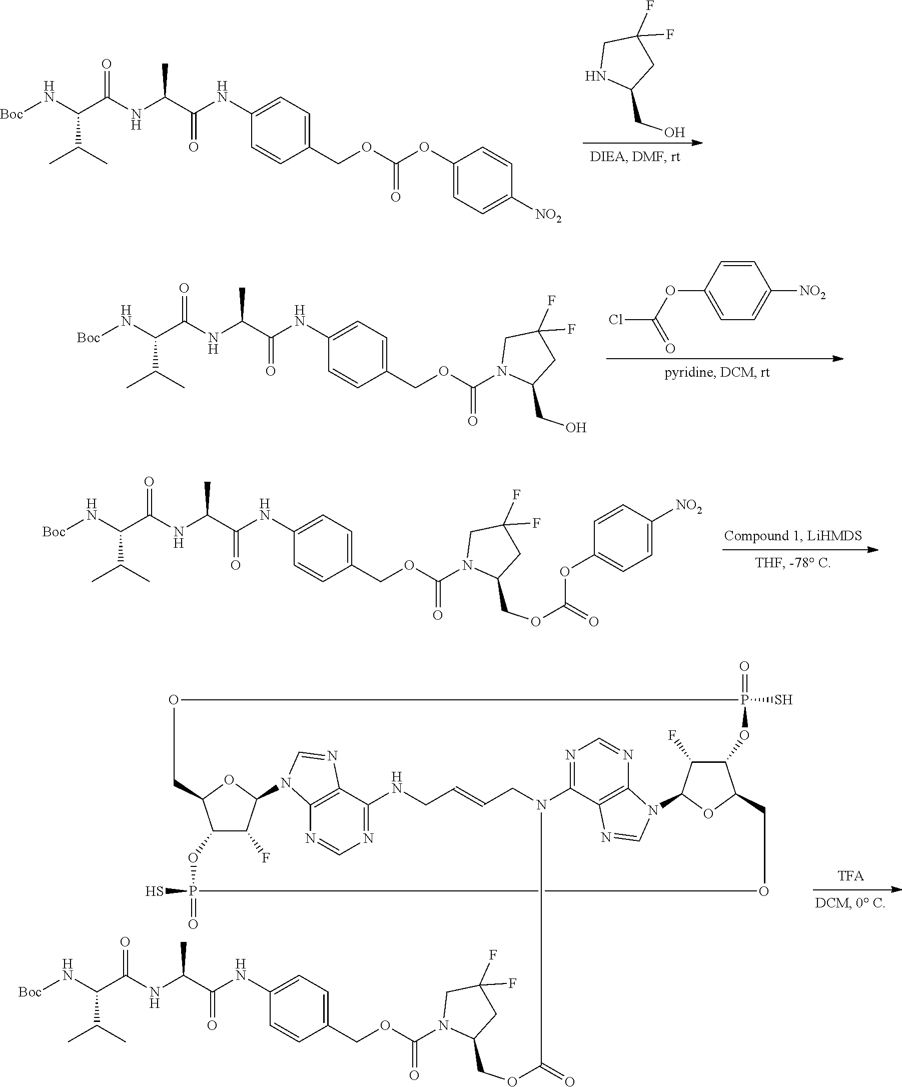

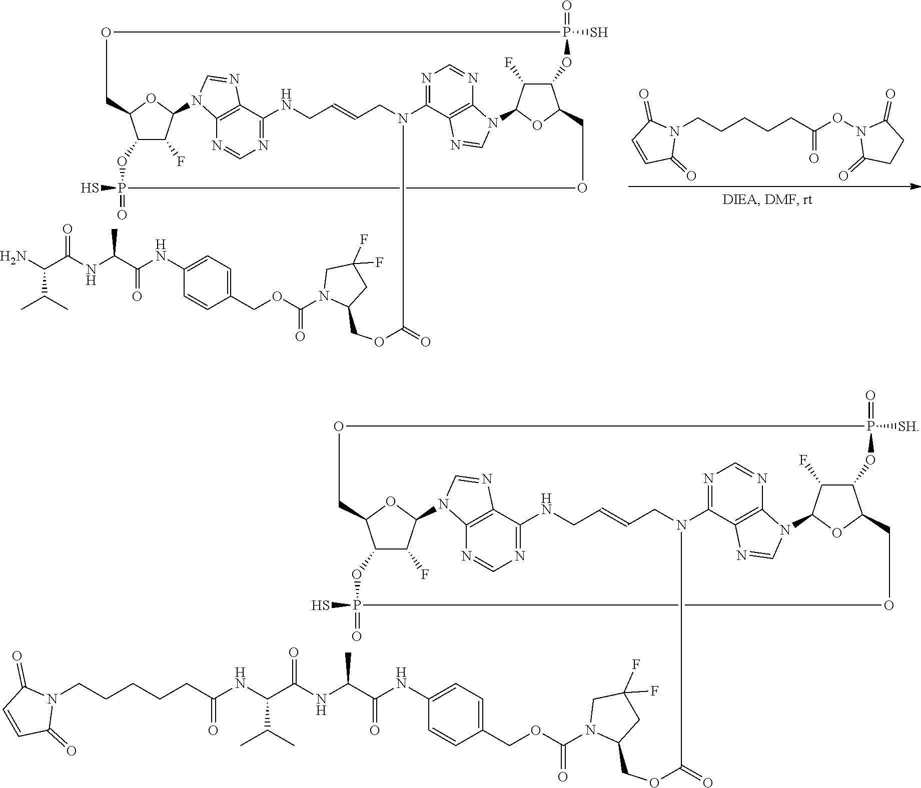 [SMILES: CC(C)[C@H](N)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of {4-[(2S)-2-[(2S)-2-{[(tert-butoxy)carbonyl]amino}-3-methylbutanamido]propanamido]phenyl}methyl (2S)-4,4-difluoro-2-(hydroxymethyl)pyrrolidine-1-carboxylate

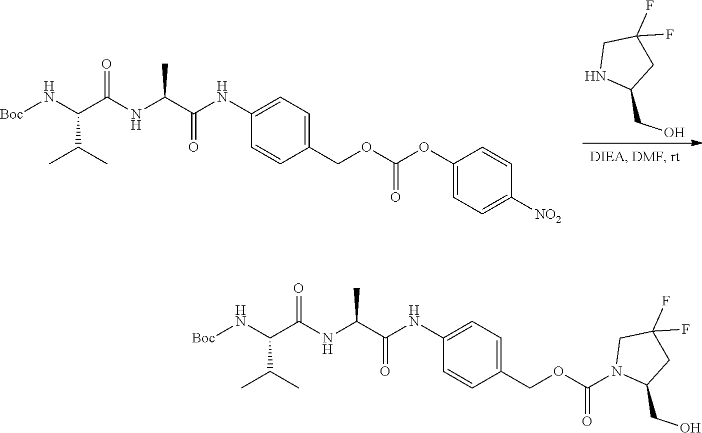 [SMILES: CC(C)[C@H](NC(=O)OC(C)(C)C)C(=O)N[C@@H](C)C(=O)Nc1ccc(COC(=O)Oc2ccc([N+](=O)[O-])cc2)cc1 ; OC[C@@H]1CC(F)(F)CN1 ; CC(C)[C@H](NC(=O)OC(C)(C)C)C(=O)N[C@@H](C)C(=O)Nc1ccc(COC(=O)N2CC(F)(F)C[C@H]2CO)cc1]

DIEA (347.07 mg, 2.685 mmol) was added to a stirred solution of {4-[(2S)-2-[(2S)-2-[(tert-butoxycarbonyl)amino]-3-methylbutanamido]propanamido]phenyl}methyl 4-nitrophenyl carbonate (500 mg, 0.895 mmol) and [(2S)-4,4-difluoropyrrolidin-2-yl]methanol (122.75 mg, 0.895 mmol) in DMF (5 mL) at 25° C. The resulting mixture was stirred at 25° C. for 2 h. The resulting mixture was diluted with EtOAc and washed with water. The resulting mixture was concentrated under reduced pressure. The residue was purified by silica gel column chromatography, eluted with DCM/MeOH (15/1) to afford {4-[(2S)-2-[(2S)-2-{[(tert-butoxy)carbonyl]amino}-3-methylbutanamido]propanamido]phenyl}methyl (2S)-4,4-difluoro-2-(hydroxymethyl)pyrrolidine-1-carboxylate (480 mg) as a white solid. LC-MS (ESI): 557.2 [M+H]⁺.

### Synthesis of {4-[(2S)-2-[(2S)-2-{[(tert-butoxy)carbonyl]amino}-3-methylbutanamido]propanamido]phenyl}methyl (2S)-4,4-difluoro-2-({[(4-nitrophenoxy)carbonyl]oxy}methyl)pyrrolidine-1-carboxylate

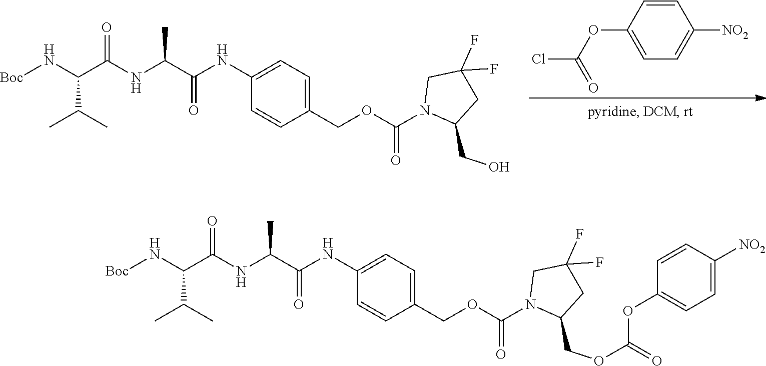 [SMILES: CC(C)[C@H](NC(=O)OC(C)(C)C)C(=O)N[C@@H](C)C(=O)Nc1ccc(COC(=O)N2CC(F)(F)C[C@H]2CO)cc1 ; O=C(Cl)Oc1ccc([N+](=O)[O-])cc1 ; CC(C)[C@H](NC(=O)OC(C)(C)C)C(=O)N[C@@H](C)C(=O)Nc1ccc(COC(=O)N2CC(F)(F)C[C@H]2COC(=O)Oc2ccc([N+](=O)[O-])cc2)cc1]

Pyridine (136.43 mg, 1.724 mmol) was added to a stirred solution of {4-[(2S)-2-[(2S)-2-{[(tert-butoxy)carbonyl]amino}-3-methylbutanamido]propanamido]phenyl}methyl (2S)-4,4-difluoro-2-(hydroxymethyl)pyrrolidine-1-carboxylate (480 mg, 0.862 mmol) and 4-nitrophenyl carbonochloridate (347.64 mg, 1.724 mmol) in DCM (10 mL) at 25° C. The resulting mixture was stirred at 25° C. for 14 h. The resulting mixture was concentrated under reduced pressure. The residue was purified by silica gel column chromatography, eluted with PE/EtOAc (1/2) to afford {4-[(2S)-2-[(2S)-2-{[(tert-butoxy)carbonyl]amino}-3-methylbutanamido]propanamido]phenyl}methyl (2S)-4,4-difluoro-2-({[(4-nitrophenoxy)carbonyl]oxy}methyl)pyrrolidine-1-carboxylate (600 mg) as a white solid. LC-MS (ESI): 722.2 [M+H]⁺.

### Synthesis of [(2S)-1-[({4-[(2S)-2-[(2S)-2-{[(tert-butoxy)carbonyl]amino}-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

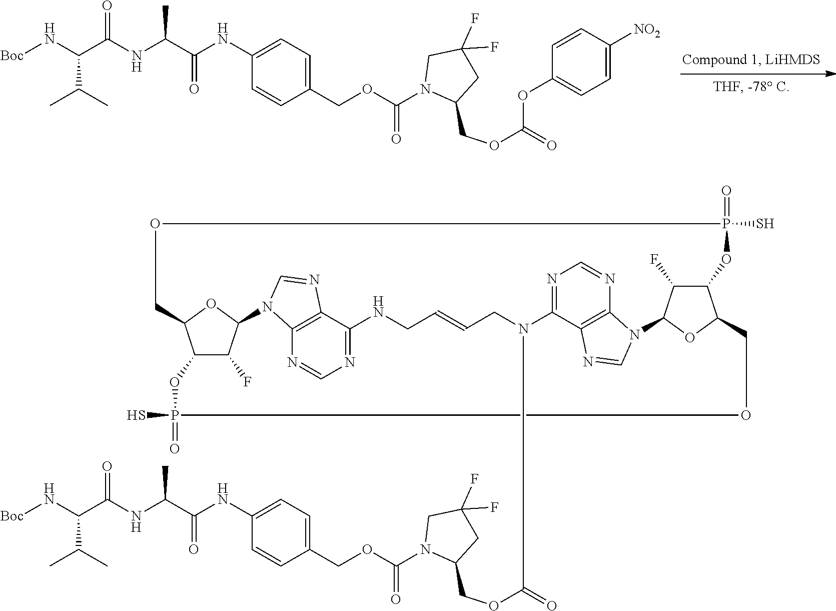 [SMILES: CC(C)[C@H](NC(=O)OC(C)(C)C)C(=O)N[C@@H](C)C(=O)Nc1ccc(COC(=O)N2CC(F)(F)C[C@H]2COC(=O)Oc2ccc([N+](=O)[O-])cc2)cc1 ; CC(C)[C@H](NC(=O)OC(C)(C)C)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

LiHMDS (0.40 mL, 0.402 mmol) was added to a stirred solution of Compound 1 in THF (5 mL) at −78° C. under nitrogen atmosphere. The resulting mixture was stirred at −78° C. for 30 min under nitrogen atmosphere. Then {4-[(2S)-2-[(2S)-2-{[(tert-butoxy)carbonyl]amino}-3-methylbutanamido]propanamido]phenyl}methyl (2S)-4,4-difluoro-2-({[(4-nitrophenoxy)carbonyl]oxy}methyl)pyrrolidine-1-carboxylate (48 mg, 0.067 mmol) was added. The resulting mixture was slowly warmed to 0° C. over 1 h under nitrogen atmosphere. The reaction was quenched with AcOH. The resulting mixture was concentrated under reduced pressure. The residue was purified by Prep-TLC (DCM (1% TEA)/MeOH (6/1)) to afford [(2S)-1-[({4-[(2S)-2-[(2S)-2-{[(tert-butoxy)carbonyl]amino}-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (44 mg) as a white solid. LC-MS (ESI): 1329.3 [M+H]⁺.

### Synthesis of [(2S)-1-[({4-[(2S)-2-[(2S)-2-amino-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

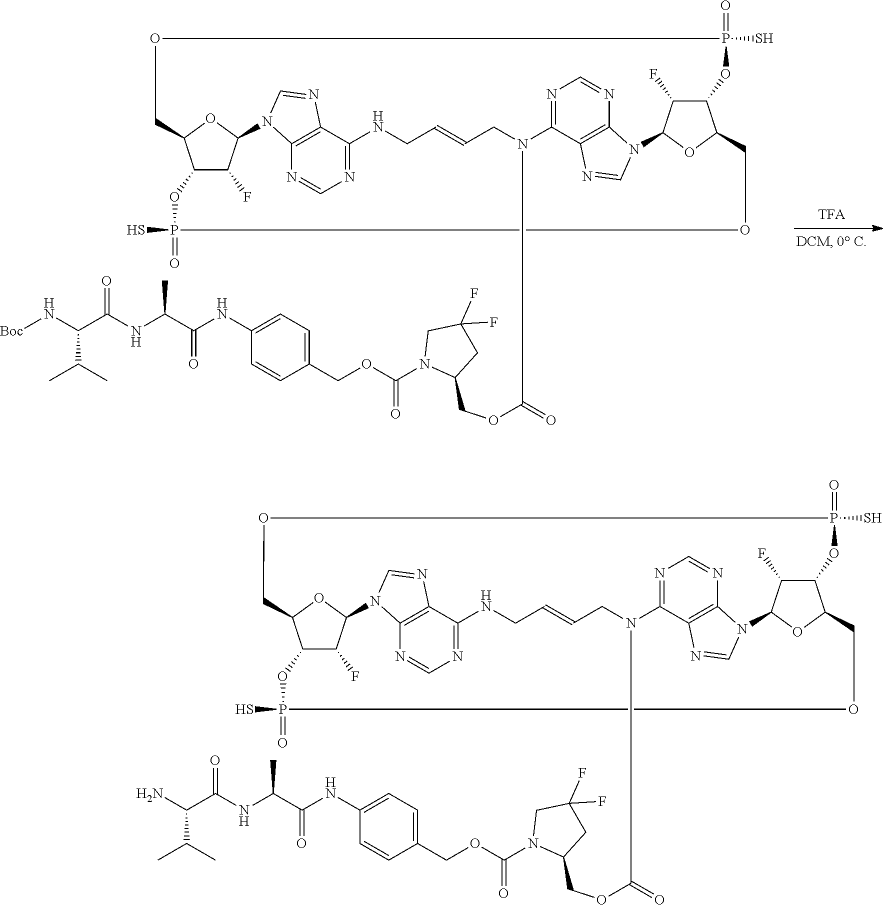 [SMILES: CC(C)[C@H](NC(=O)OC(C)(C)C)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](N)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

TFA (0.6 mL) was added to a stirred solution of [(2S)-1-[({4-[(2S)-2-[(2S)-2-[(tert-butoxycarbonyl)amino]-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (44 mg, 0.033 mmol) in DCM (2.4 mL) at 0° C. under nitrogen atmosphere. The resulting mixture was stirred at 0° C. for 2 h under nitrogen atmosphere.

The resulting mixture was concentrated under reduced pressure to obtain the crude product of [(2S)-1-[({4-[(2S)-2-[(2)-2-amino-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a colorless oil. LC-MS (ESI): 1229.2 [M+H]⁺.

### Synthesis of [(2S)-1-[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP28)

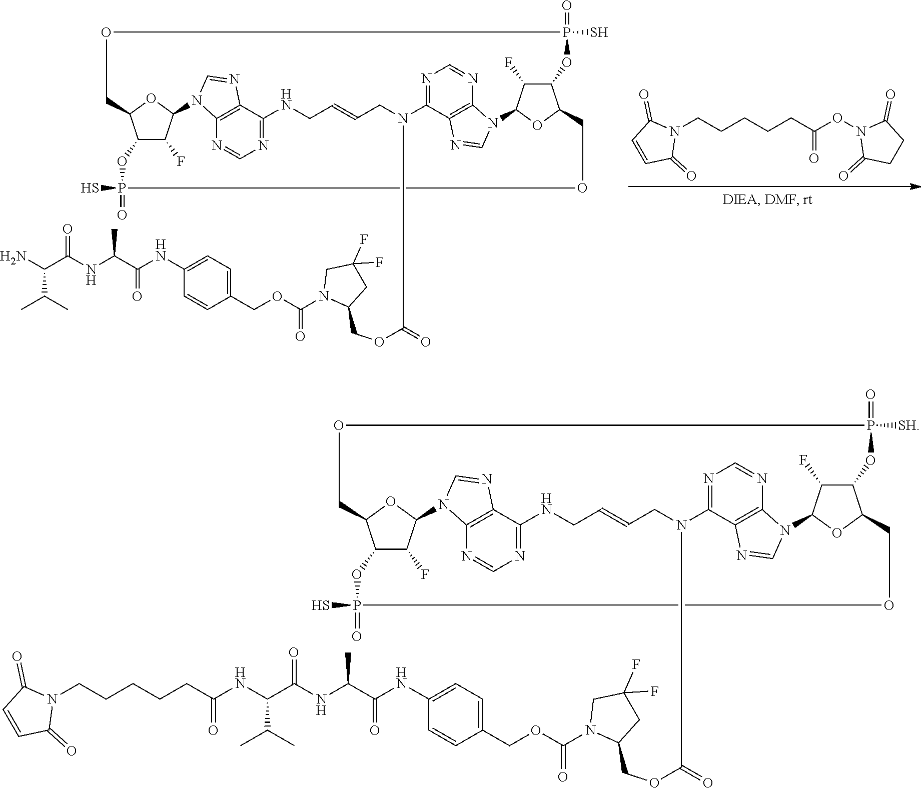 [SMILES: CC(C)[C@H](N)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

DIEA (25.24 mg, 0.198 mmol) was added to a mixture of [(2S)-1-[({4-[(2S)-2-[(2S)-2-amino-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg, 0.033 mmol) and 2,5-dioxopyrrolidin-1-yl 6-(2,5-dioxopyrrol-1-yl)hexanoate (10.03 mg, 0.033 mmol) in DMF (2 mL) at 25° C. under nitrogen atmosphere. The resulting mixture was stirred at 25° C. for 4 h under nitrogen atmosphere. The resulting mixture was concentrated under reduced pressure. The residue was purified by Prep-TLC DCM (1% TEA)/MeOH (7/1) and Prep-HPLC with the following conditions (Column: Xbridge Prep OBD C18 Column, 19*250 mm, 5 μm; Mobile Phase A: Water(50 mmol HCO₂NH₄), Mobile Phase B: MeCN; Flow rate: 25 mL/min; Gradient: 25% B to 35% B in 10 min, 35% B; Wavelength: 254 nm) to afford [(2S)-1-[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP28) (12.8 mg) as an off-white solid.

LC-MS (ESI):1423.60 [M+H]⁺.

¹H NMR (400 MHz, Methanol-d₄) δ 8.94-8.90 (m, 1H), 8.78-8.73 (m, 2H), 8.17-8.00 (m, 3H), 7.65-7.59 (m, 2H), 7.31-7.27 (m, 2H), 6.81-6.76 (m, 2H), 6.49-6.44 (m, 1H), 6.33-6.29 (m, 1H), 6.06-5.87 (m, 2H), 5.85-5.57 (m, 2H), 5.17-4.94 (m, 3H), 4.69-4.58 (m, 5H), 4.55-4.46 (m, 3H), 4.46-4.36 (m, 3H), 4.34-4.22 (m, 2H), 4.22-4.02 (m, 2H), 4.01-3.97 (m, 1H), 3.80-3.76 (m, 1H), 3.66-3.57 (m, 1H), 3.51-3.43 (m, 2H), 3.33-3.16 (m, 1H), 2.56-2.36 (m, 1H), 2.34-2.26 (m, 2H), 2.25-1.92 (m, 2H), 1.70-1.53 (m, 4H), 1.52-1.42 (m, 3H), 1.38-1.21 (m, 2H), 1.03-0.94 (m, 6H).

### 24. Synthesis of LP26

The synthesis of LP26 is shown below:

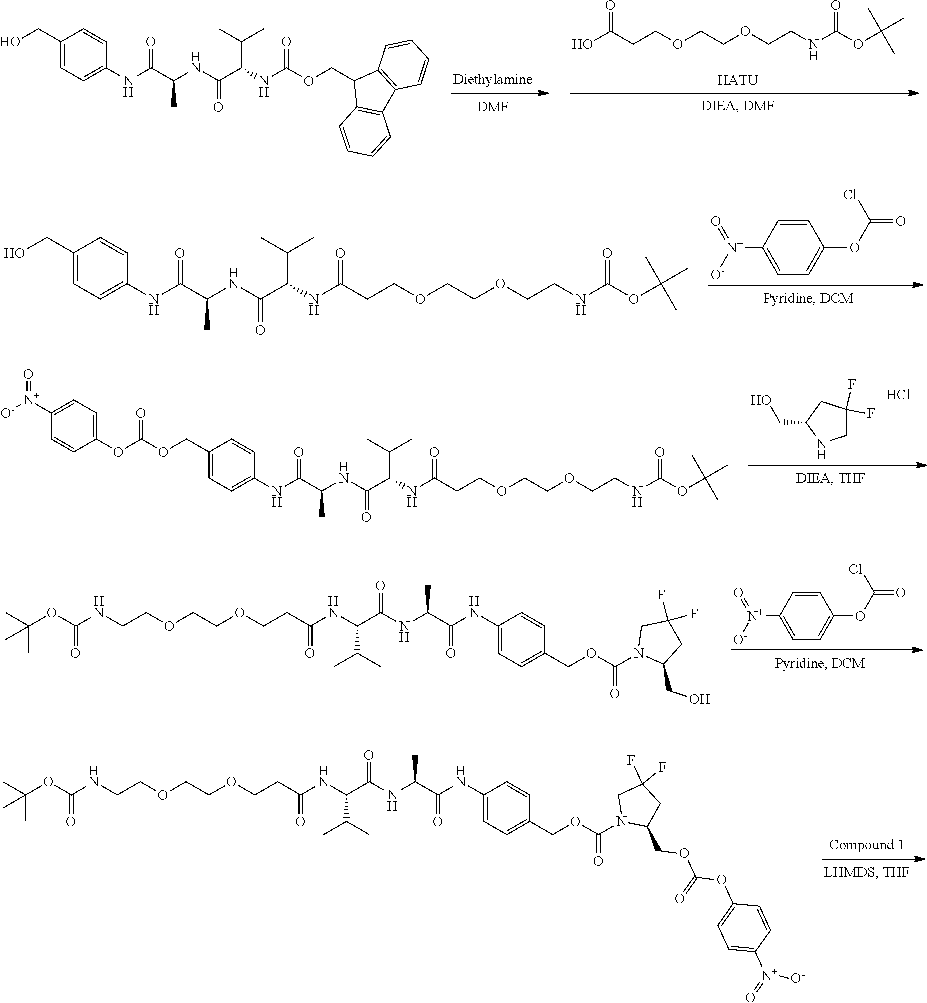

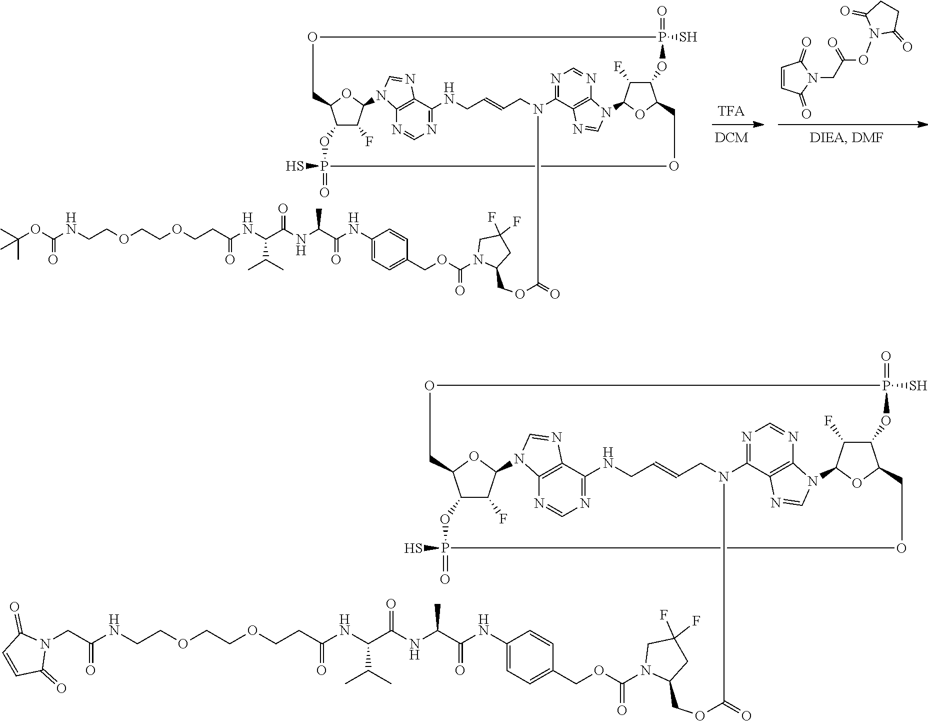

### Synthesis of tert-butyl N-{2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamate

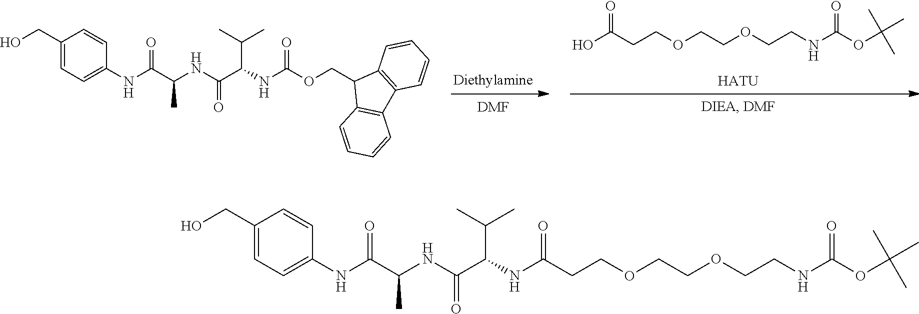

To a solution of (9H-fluoren-9-yl)methyl N-[(1S)-1-{[(1S)-1-{[4-(hydroxymethyl)phenyl]carbamoyl}ethyl]carbamoyl}-2-methylpropyl]carbamate (410 mg, 0.795 mmol) in DMF (5 mL) was added diethylamine (0.828 mL, 7.95 mmol). The mixture was stirred for 1 h. The resulting mixture was concentrated and azeotroped with toluene under reduced pressure. To the residue in DMF (5 mL) were added 3-[2-(2-{[(tert-butoxy)carbonyl]amino}ethoxy)ethoxy]propanoic acid (250 mg, 0.901 mmol), DIEA (0.278 mL, 1.59 mmol) and HATU (454 mg, 1.19 mmol). The mixture was stirred for 90 min. The resulting mixture was concentrated under reduced pressure. The residue was diluted with EtOAc and water. The organic layer was separated and washed with brine, dried over Na₂SO₄, and concentrated under reduced pressure. The residue was purified by silica gel column chromatography (EtOAc/heptane=1/19 to 1/10) to give crude material. The crude material was purified by NH silica gel column chromatography (EtOAc/heptane=1/1 to 1/0, then EtOAc/MeOH=10/1) to give tert-butyl N-{2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamate (354.4 mg). LC-MS (ESI): 553.5 [M+H]⁺.

### Synthesis of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl 4-nitrophenyl carbonate

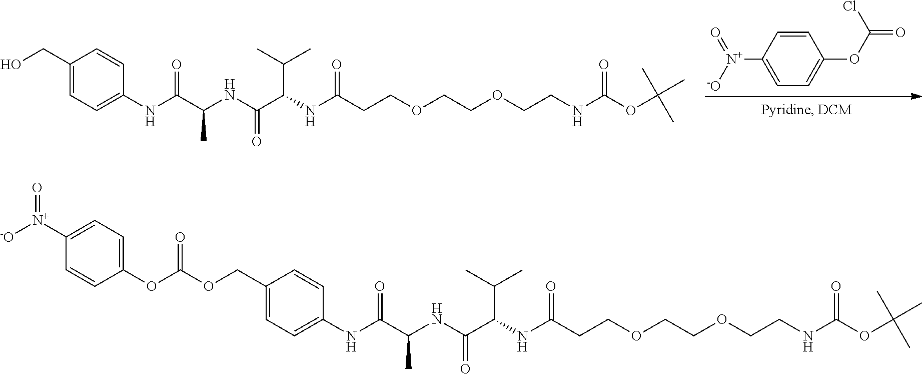

To a solution of tert-butyl N-{2-[2-(2-{[(1S)-1-{[(1S)-1-{[4-(hydroxymethyl)phenyl]carbamoyl}ethyl]carbamoyl}-2-methylpropyl]carbamoyl}ethoxy)ethoxy]ethyl}carbamate (354.4 mg, 0.641 mmol) in DCM (15 mL) were added 4-nitrophenyl carbonochloridate (323 mg, 1.60 mmol) and pyridine (0.130 mL, 1.60 mmol). The mixture was stirred for 1 h. The resulting mixture was diluted with EtOAc and 10% aqueous citric acid. The organic layer was separated and washed with brine, dried over Na₂SO₄, and concentrated under reduced pressure. The residue was purified by silica gel column chromatography (EtOAc/heptane=1/1 to 1/0) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl 4-nitrophenyl carbonate (412.2 mg). LC-MS (ESI): 718.4 [M+H]⁺.

### Synthesis of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-(hydroxymethyl)pyrrolidine-1-carboxylate

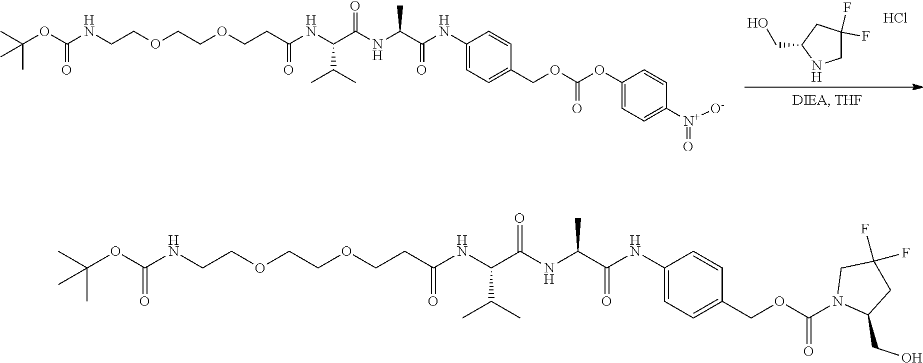 [SMILES: CC(C)[C@H](NC(=O)CCOCCOCCNC(=O)OC(C)(C)C)C(=O)N[C@@H](C)C(=O)Nc1ccc(COC(=O)Oc2ccc([N+](=O)[O-])cc2)cc1 ; OC[C@@H]1CC(F)(F)CN1 ; Cl ; CC(C)[C@H](NC(=O)CCOCCOCCNC(=O)OC(C)(C)C)C(=O)N[C@@H](C)C(=O)Nc1ccc(COC(=O)N2CC(F)(F)C[C@H]2CO)cc1]

To a solution of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl 4-nitrophenyl carbonate (150 mg, 0.209 mmol) in THF (4 mL) and DIEA (0.183 mL, 1.05 mmol) was added [(2S)-4,4-difluoropyrrolidin-2-yl]methanol hydrochloride (43.5 mg, 0.251 mmol). The mixture was stirred for 77 h. The resulting mixture was concentrated under reduced pressure. The residue was purified by NH silica gel column chromatography (EtOAc/heptane=1/1 to 1/0, then EtOAc/MeOH=10/1) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-(hydroxymethyl)pyrrolidine-1-carboxylate (138.3 mg). LC-MS (ESI): 716.5 [M+H]⁺.

### Synthesis of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-({[(4-nitrophenoxy)carbonyl]oxy}methyl)pyrrolidine-1-carboxylate

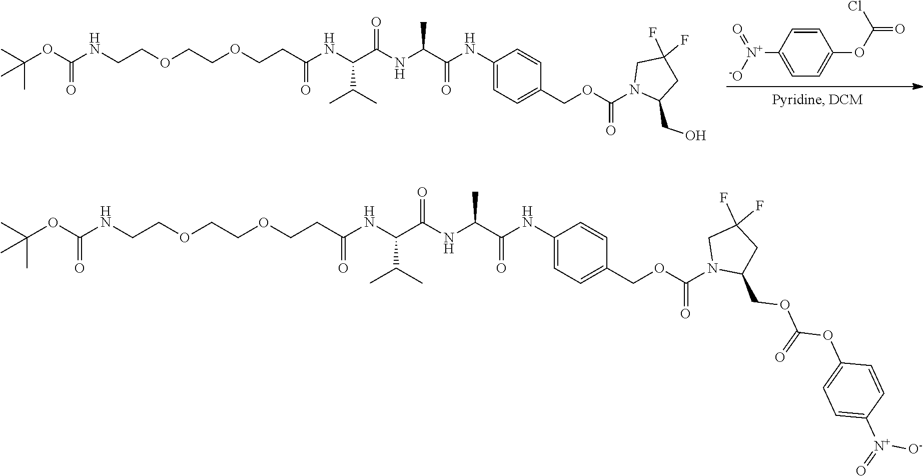

To a solution of {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-(hydroxymethyl)pyrrolidine-1-carboxylate (138.3 mg, 0.193 mmol) in DCM (5 mL) were added 4-nitrophenyl carbonochloridate (78 mg, 0.386 mmol) and pyridine (0.031 mL, 0.386 mmol). The mixture was stirred for 1 h. To the mixture were added pyridine (0.016 mL, 0.193 mmol) and 4-nitrophenyl carbonochloridate (38.9 mg, 0.193 mmol). The mixture was stirred for 4 h. The resulting mixture was diluted with EtOAc and 10% aqueous citric acid. The organic layer was separated, washed with brine, dried over Na₂SO₄, and concentrated under reduced pressure. The residue was purified by silica gel column chromatography (EtOAc/heptane=1/1 to 1/0, then EtOAc/MeOH=10/1) to give {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-({[(4-nitrophenoxy)carbonyl]oxy}methyl)pyrrolidine-1-carboxylate (173.8 mg). LC-MS (ESI): 881.5 [M+H]⁺.

### Synthesis of [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

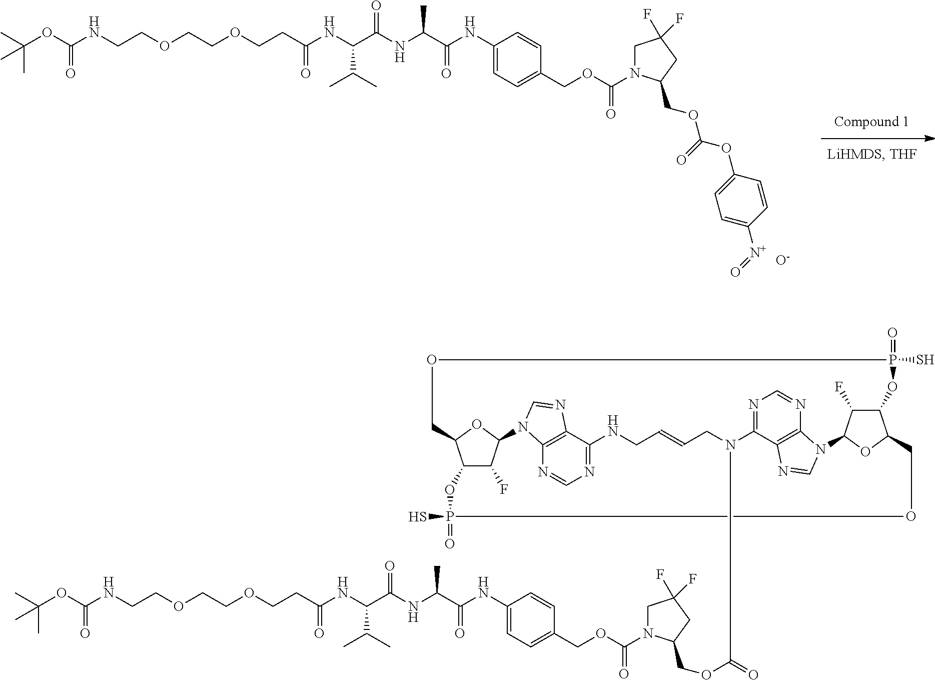

To a suspension of Compound 1 (50 mg, 0.059 mmol) in THF (5 mL) at room temperature was added LiHMDS (0.586 mmol). The resulting mixture was stirred for 30 min at room temperature and cooled to −78° C. To the above mixture was added {4-[(2S)-2-[(2S)-2-{3-[2-(2-{[(tert-butoxy)carbonyl]amino}ethoxy)ethoxy]propanamido}-3-methylbutanamido]propanamido]phenyl}methyl (2S)-4,4-difluoro-2-({[(4-nitrophenoxy)carbonyl]oxy}methyl)pyrrolidine-1-carboxylate (54.2 mg, 0.062 mmol) in THF (0.5 mL) with THF rinse (0.5 mL) and stirred at −78° C. for 10 min. The resulting mixture was warmed to room temperature and stirred for 3 h. The reaction was quenched with AcOH (0.252 mL, 4.40 mmol) and stirred for 10 min. The resulting mixture was concentrated under reduced pressure. The residue was purified by ODS silica gel column chromatography (H2O/MeCN including 0.1% formic acid=95/25 to 20/80) to give [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]-4,4-difluoropyrrolidin-2-yl]methyl(1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (41.1 mg). LC-MS (ESI): 1488.4 [M+H]⁺.

### Synthesis of [(2S)-1-[({4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP26)

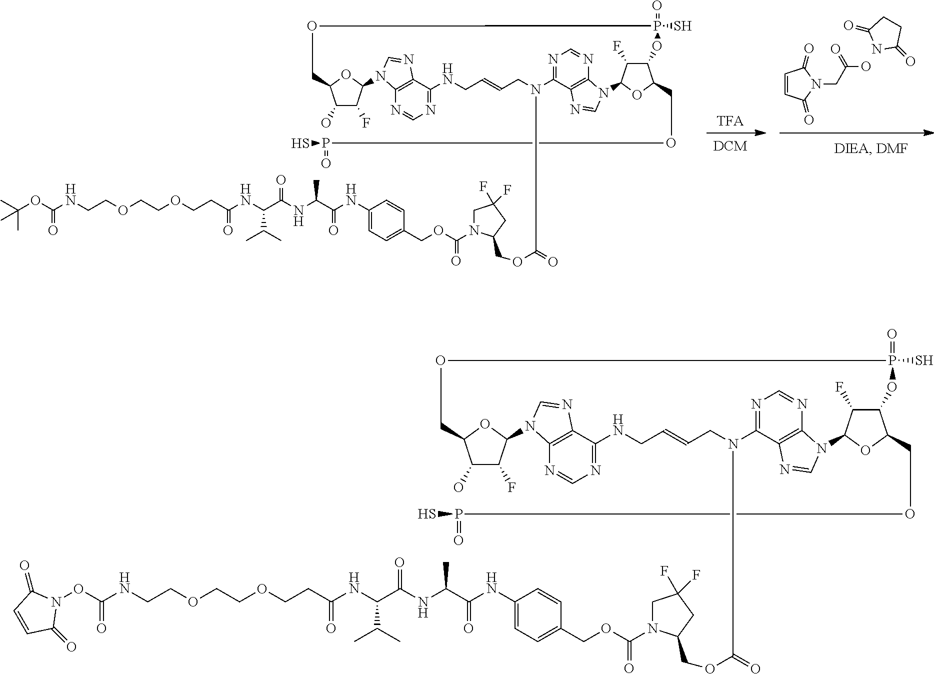

To a suspension of [(2S)-1-[({4-[(2S)-2-[(2S)-2-{3-[2-(2-{[(tert-butoxy)carbonyl]amino}ethoxy)ethoxy]propanamido}-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (21.1 mg, 0.014 mmol) in DCM (2 mL) at 0° C. was added TFA (200 mL, 2.60 mmol). The mixture was stirred for 30 min at room temperature. The resulting mixture was concentrated and azeotroped with toluene under reduced pressure. To the residue in DMF (1 mL) at 0° C. were added DIEA (0.025 mL, 0.142 mmol) and 2,5-dioxopyrrolidin-1-yl 2-(2,5-dioxo-2,5-dihydro-1H-pyrrol-1-yl)acetate (14.30 mg, 0.057 mmol). The mixture was stirred for 2 h at room temperature. The resulting mixture was concentrated under reduced pressure. The residue was purified by ODS silica gel column chromatography (H2O/MeCN including 0.1% formic acid=95/25 to 20/80) to give [(2S)-1-[({4-[(2S)-2-[(2S)-2-[3-(2-{2-[2-(2,5-dioxo-2,5-dihydro-1H-pyrrol-1-yl)acetamido]ethoxy}ethoxy)propanamido]-3-methylbutanamido]propanamido]phenyl}methoxy)carbonyl]-4,4-difluoropyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP26) (6.85 mg).

LC-MS (ESI): 1525.6 [M+H]⁺.

Rotamers were observed in the ¹H NMR spectrum. ¹H NMR (700 MHz, DMSO-d₆) δ ppm 9.91-10.11 (m, 2H) 8.81 (br s, 1H) 8.59-8.72 (m, 2H) 8.31-8.39 (m, 1H) 8.17-8.27 (m, 2H) 8.08-8.17 (m, 1H) 7.84-7.92 (m, 1H) 7.65-7.75 (m, 1H) 7.55-7.62 (m, 2H) 7.45-7.50 (m, 1H) 7.24 (br s, 2H) 7.22 (s, 1H) 7.15 (s, 1H) 7.05-7.11 (m, 3H) 6.92-6.96 (m, 1H) 6.46-6.53 (m, 1H) 6.35-6.44 (m, 1H) 5.78-5.94 (m, 2H) 5.69 (br s, 1H) 5.54-5.61 (m, 1H) 5.29-5.36 (m, 1H) 5.23 (br s, 1H) 5.08-5.16 (m, 1H) 5.01-5.08 (m, 1H) 4.97 (br s, 2H) 4.90 (br s, 1H) 4.71 (br d, J=12.76 Hz, 1H) 4.65 (br s, 1H) 4.56 (br d, J=11.44 Hz, 1H) 4.42 (br s, 1H) 4.33-4.41 (m, 5H) 4.27-4.33 (m, 3H) 4.18-4.27 (m, 4H) 4.11-4.16 (m, 2H) 4.02 (s, 2H) 3.81 (br d, J=5.72 Hz, 1H) 3.64-3.77 (m, 4H) 3.56-3.63 (m, 3H) 3.44-3.50 (m, 4H) 3.39 (t, J=5.72 Hz, 2H) 3.19 (q, J=5.72 Hz, 2H) 2.45-2.48 (m, 1H) 2.36-2.41 (m, 1H) 1.88-2.05 (m, 3H) 1.28-1.36 (m, 4H) 1.19-1.28 (m, 5H) 0.86-0.88 (m, 3H) 0.83 (br d, J=6.60 Hz, 3H).

### 25. Synthesis of LP27

The synthesis of LP27 is shown below:

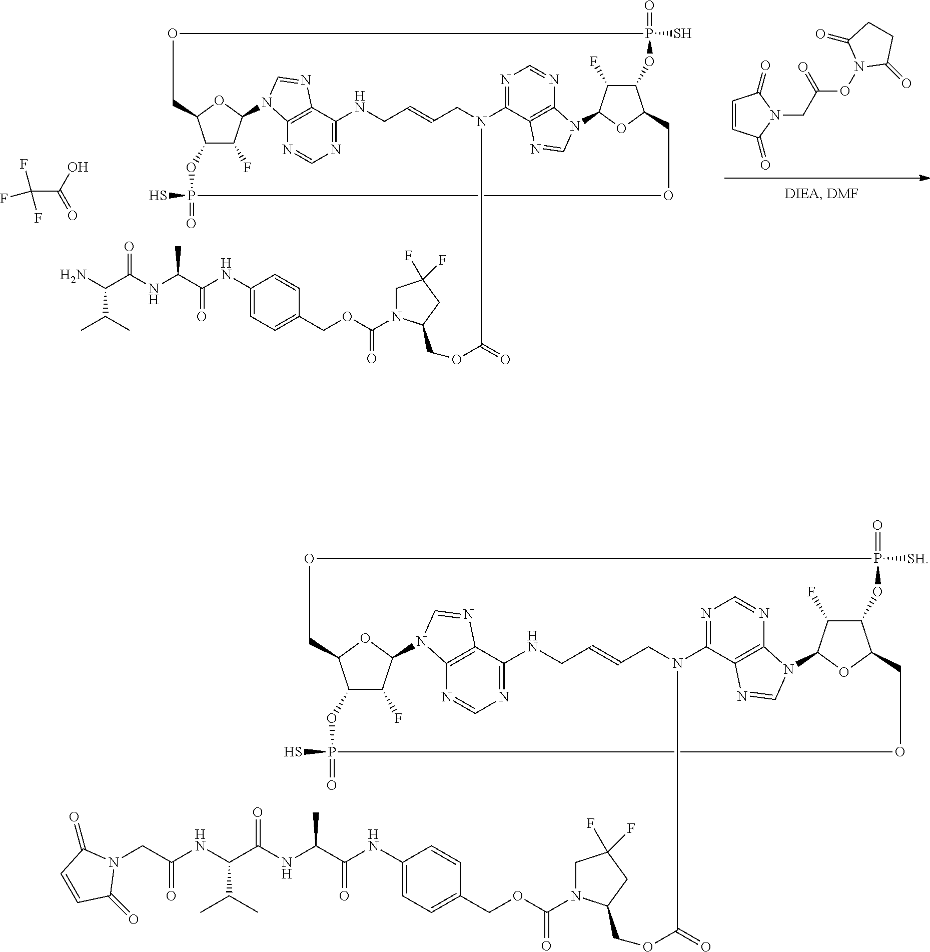 [SMILES: O=C(O)C(F)(F)F ; CC(C)[C@H](N)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CN1C(=O)C=CC1=O)ON1C(=O)CCC1=O ; CC(C)[C@H](NC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To the solution of [(25)-1-[({4-[(2S)-2-[(2S)-2-amino-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate trifluoroacetic acid salt (15 mg, 0.011 mmol) and DIEA (9.75 μl, 0.056 mmol) in DMF (2 ml, 25.829 mmol) was added 2,5-dioxopyrrolidin-1-yl 2-(2,5-dioxo-2,5-dihydro-1H-pyrrol-1-yl)acetate (4.22 mg, 0.017 mmol) and stirred at room temperature for 1 hour. The resulting mixture was concentrated under reduced pressure. The residue was purified by reverse phase silica gel chromatography (ODS C18 column, H₂O/MeCN/HCOOH=95/5/0.1 to 40/60/0.1) to give [(25)-1-[({4-[(25)-2-[(25)-2-[2-(2,5-dioxo-2,5-dihydro-1H-pyrrol-1-yl)acetamido]-3-methylbutanamido]propanamido]phenyl}methoxy)carbonyl]-4,4-difluoropyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP27) as white powder.

LC-MS (ESI) [M+H]⁺=1366.30.

¹H NMR (396 MHz, DMSO-d₆) δ ppm 0.80 (br d, J=16.76 Hz, 4H) 0.82 (br d, J=16.76 Hz, 3H) 1.17-1.23 (m, 4H) 1.27 (br d, J=5.89 Hz, 4H) 1.82-2.04 (m, 2H) 4.08 (s, 3H) 4.17-4.28 (m, 5H) 4.30-4.42 (m, 3H) 4.43-4.74 (m, 3H) 4.93 (br s, 1H) 5.11 (br s, 1H) 5.14-5.33 (m, 1H) 5.38 (br d, J=1.36 Hz, 1H) 5.46-5.60 (m, 1H) 5.60-5.81 (m, 1H) 5.82-5.96 (m, 1H) 6.26-6.53 (m, 1H) 7.05 (s, 2H) 7.21 (br d, J=7.25 Hz, 2H) 7.53 (br d, J=8.16 Hz, 2H) 8.22 (br d, J=8.61 Hz, 1H) 8.28 (br d, J=6.80 Hz, 1H) 8.32-8.39 (m, 1H) 8.52-8.69 (m, 2H) 8.69-8.83 (m, 1H) 9.88-10.11 (m, 1H).

### 26. Synthesis of LP24

The synthesis of LP24 (SEQ ID NO: 144) is shown below:

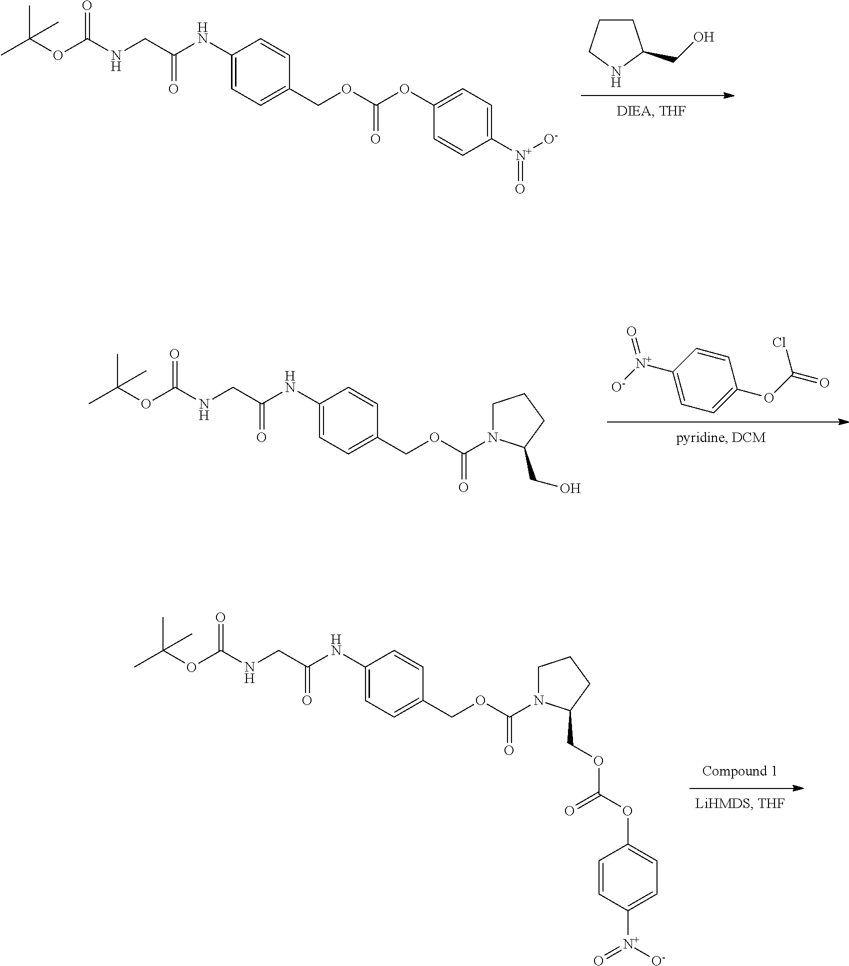 [SMILES: CC(C)(C)OC(=O)NCC(=O)Nc1ccc(COC(=O)Oc2ccc([N+](=O)[O-])cc2)cc1 ; OC[C@@H]1CCCN1 ; CC(C)(C)OC(=O)NCC(=O)Nc1ccc(COC(=O)N2CCC[C@H]2CO)cc1 ; O=C(Cl)Oc1ccc([N+](=O)[O-])cc1 ; CC(C)(C)OC(=O)NCC(=O)Nc1ccc(COC(=O)N2CCC[C@H]2COC(=O)Oc2ccc([N+](=O)[O-])cc2)cc1]

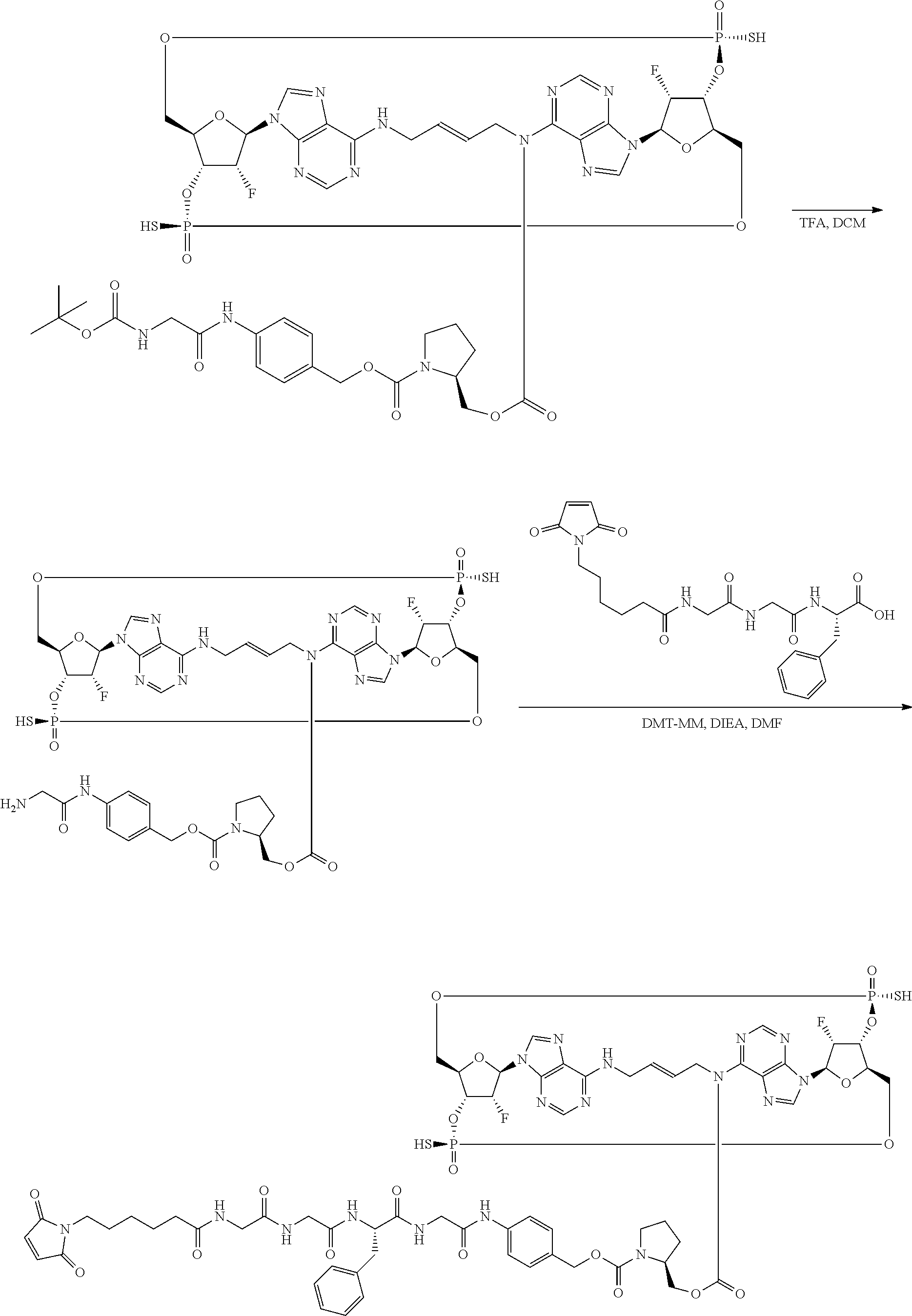

### Synthesis of [4-(2-{[(tert-butoxy)carbonyl]amino}acetamido)phenyl]methyl (2S)-2-(hydroxymethyl)pyrrolidine-1-carboxylate

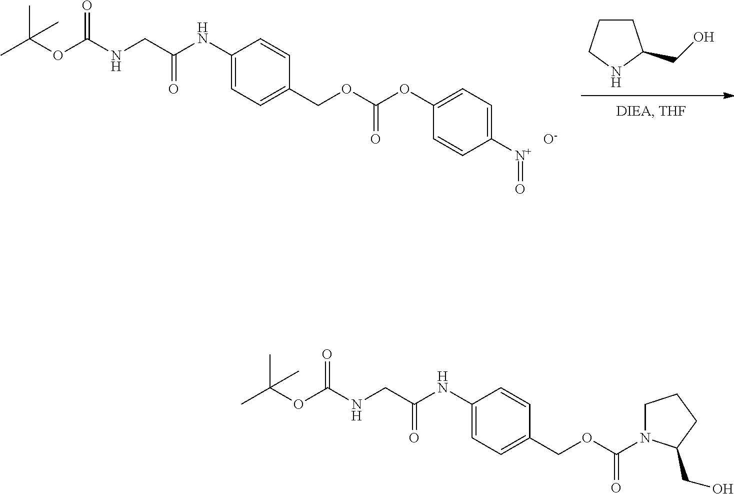

[(2S)-pyrrolidin-2-yl]methanol (272 mg, 2.69 mmol) was added to a solution of [4-(2-{[(tert-butoxy)carbonyl]amino}acetamido)phenyl]methyl 4-nitrophenyl carbonate (1.00 g, 2.25 mmol) and DIEA (1.45 g, 11.2 mmol) in THF (41.8 mL) at room temperature. The solution was stirred at room temperature for 30 min. The resulting mixture was concentrated under reduced pressure. The residue was purified by silica gel column chromatography, eluted with EtOAc/heptane (1/3 to 1/0) to give [4-(2-{[(tert-butoxy)carbonyl]amino}acetamido)phenyl]methyl (2S)-2-(hydroxymethyl)pyrrolidine-1-carboxylate (873 mg). LC-MS (ESI): 408.3 [M+]⁺.

### Synthesis of [4-(2-{[(tert-butoxy)carbonyl]amino}acetamido)phenyl]methyl (2S)-2-({[(4-nitrophenoxy)carbonyl]oxy}methyl)pyrrolidine-1-carboxylate

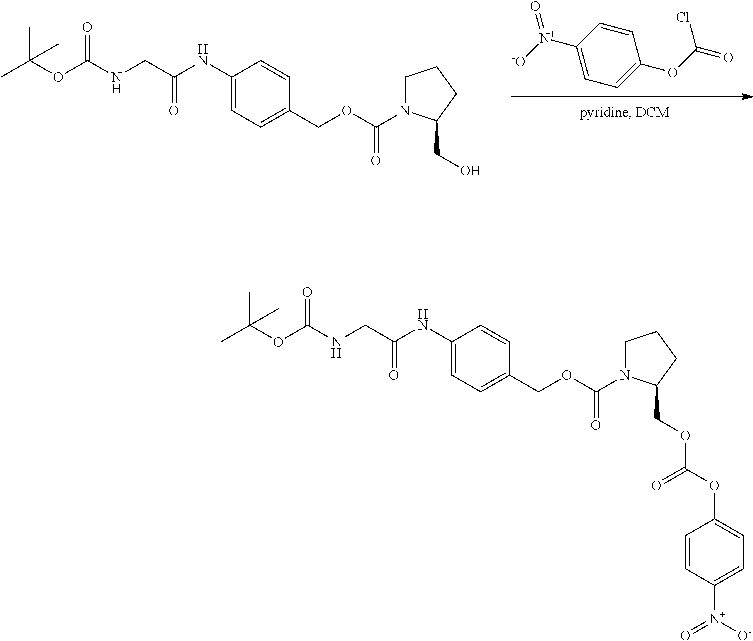

Pyridine (164 mg, 2.08 mmol) was added to a solution of [4-(2-{[(tert-butoxy)carbonyl]amino}acetamido)phenyl]methyl (2S)-2-(hydroxymethyl)pyrrolidine-1-carboxylate (423 mg, 1.04 mmol) and 4-nitrophenyl carbonochloridate (418 mg, 2.08 mmol) in DCM (17.4 mL) at room temperature. The solution was stirred at room temperature for 1 h. The resulting mixture was diluted with EtOAc and 10% aqueous citric acid. The organic layer was separated and washed with brine and concentrated under reduced pressure. The residue was purified by silica gel column chromatography, eluted with EtOAc/heptane, 1/3 to 1/0 to give [4-(2-{[(tert-butoxy)carbonyl]amino}acetamido)phenyl]methyl (2S)-2-({[(4-nitrophenoxy)carbonyl]oxy}methyl)pyrrolidine-1-carboxylate (542 mg). LC-MS (ESI): 573.3 [M+H]⁺.

### Synthesis of [(2S)-1-({[4-(2-{[(tert-butoxy)carbonyl]amino}acetamido)phenyl]methoxy}carbonyl)pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

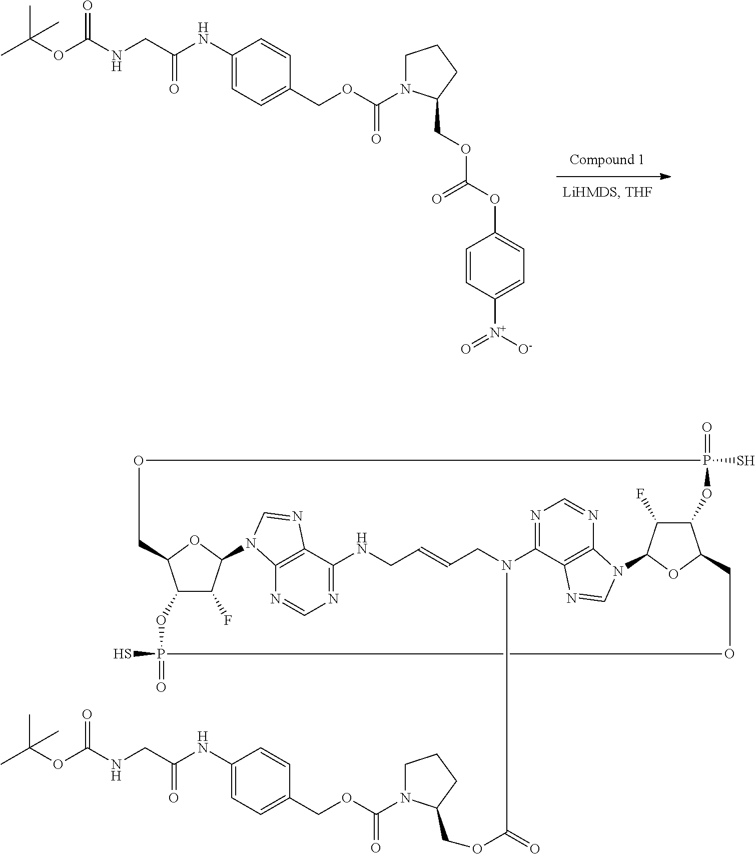 [SMILES: CC(C)(C)OC(=O)NCC(=O)Nc1ccc(COC(=O)N2CCC[C@H]2COC(=O)Oc2ccc([N+](=O)[O-])cc2)cc1 ; CC(C)(C)OC(=O)NCC(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

LiHMDS (1.17 mmol) was added to a solution Compound 1 (100 mg, 0.117 mmol) in THF (10 mL) at room temperature. The resulting mixture was stirred for 30 min at room temperature and cooled to −78° C. To the above mixture was added [4-(2-{[(tert-butoxy)carbonyl]amino}acetamido)phenyl]methyl (2S)-2-({[(4-nitrophenoxy)carbonyl]oxy}methyl)pyrrolidine-1-carboxylate (67.1 mg, 0.117 mmol) in THF (2 mL) and stirred at −78° C. for 10 min. The resulting mixture was warmed to 0° C. and stirred for 30 min. The reaction was quenched with AcOH, then warmed to room temperature and stirred for 10 min. The resulting mixture was concentrated under reduced pressure. The residue was purified by ODS silica gel column chromatography, eluted with 0.1% HCO₂H in H₂O/0.1% HCO₂H in MeCN (99/1 to 20/80) to give [(2S)-1-({[4-(2-{[(tert-butoxy)carbonyl]amino}acetamido)phenyl]methoxy}carbonyl)pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (56.7 mg). LC-MS (ESI): 1180.5 [M+H]⁺.

### Synthesis of [(2S)-1-({[4-(2-aminoacetamido)phenyl]methoxy}carbonyl)pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

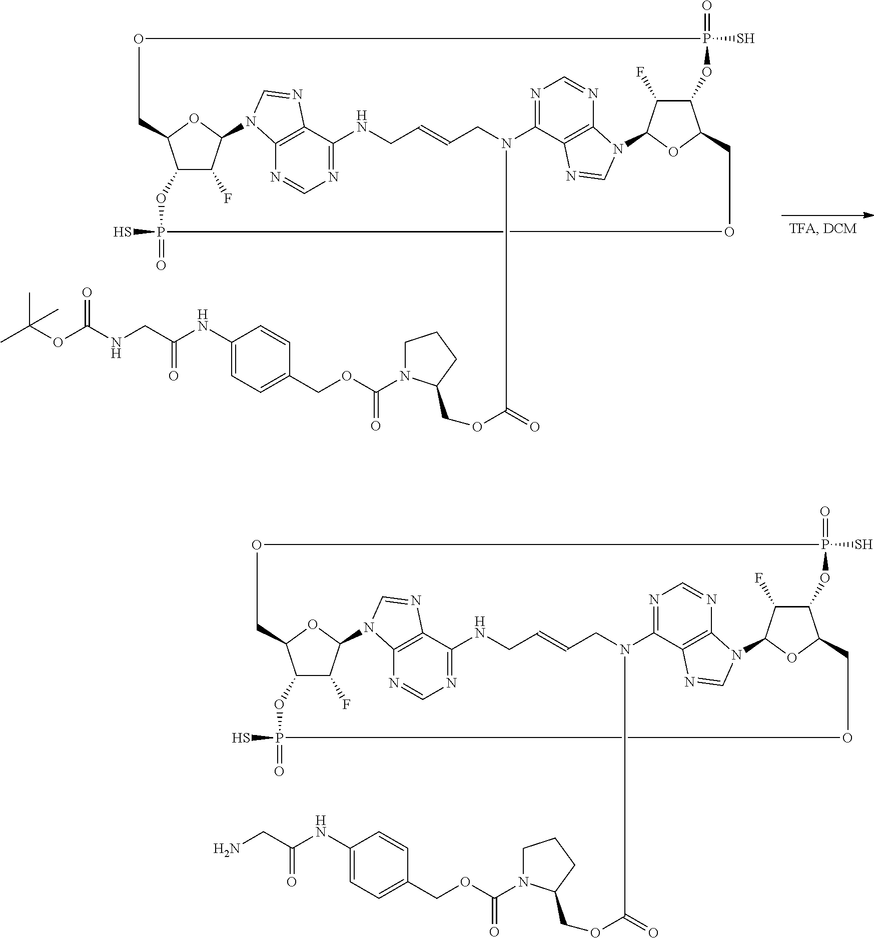

TFA (1.11 mL) was added to a solution of [(2S)-1-({[4-(2-{[(tert-butoxy)carbonyl]amino}acetamido)phenyl]methoxy}carbonyl)pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (56.7 mg, 0.048 mmol) in DCM (3.71 mL) at 0° C. The resulting mixture was stirred for 45 min at 0° C. The resulting mixture was diluted with toluene (2 mL) and concentrated under reduced pressure. The residue was purified by ODS silica gel column chromatography, eluted with 0.1% NH₃ in H₂O/0.1% NH₃ in MeCN, 95/5 to 35/65 to give [(2S)-1-({[4-(2-aminoacetamido)phenyl]methoxy}carbonyl)pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (32.1 mg). LC-MS (ESI): 1080.4 [M+H]⁺.

### Synthesis of [(2S)-1-{[(4-{2-[(2S)-2-(2-{2-[6-(2,5-dioxo-2,5-dihydro-1H-pyrrol-1-yl)hexanamido]acetamido}acetamido)-3-phenylpropanamido]acetamido}phenyl)methoxy]carbonyl}pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP24)

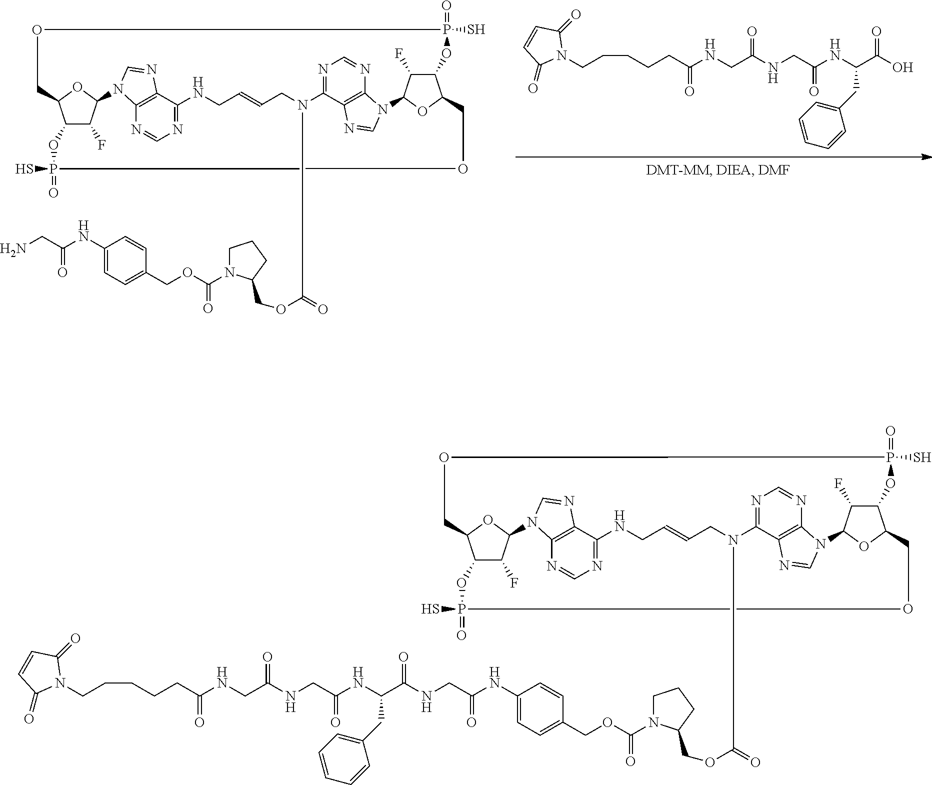 [SMILES: NCC(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; O=C(CCCCCN1C(=O)C=CC1=O)NCC(=O)NCC(=O)N[C@@H](Cc1ccccc1)C(=O)O ; "DMT-MM, DIEA, DMF" ; O=C(CCCCCN1C(=O)C=CC1=O)NCC(=O)NCC(=O)N[C@@H](Cc1ccccc1)C(=O)NCC(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

DIEA (2.4 mg, 0.019 mmol) was added to a solution of [(2S)-1-({[4-(2-aminoacetamido)phenyl]methoxy}carbonyl)pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (10 mg, 0.0093 mmol) and (2S)-2-(2-{2-[6-(2,5-dioxo-2,5-dihydro-1H-pyrrol-1-yl)hexanamido]acetamido}acetamido)-3-phenylpropanoic acid (6.6 mg, 0.014 mmol) in DMF (1.0 mL) at room temperature. The resulting mixture was cooled 0° C., and DMT-MM (4.1 mg) was added. The resulting mixture was warmed to room temperature and stirred for 10 min. Then, additional DMT-MM (0.8 mg) was added at 0° C. The resulting mixture was warmed to room temperature and stirred for 10 min. The mixture was directly purified by ODS silica gel column chromatography, eluted with 0.1% HCO₂H in H₂O/0.1% HCO₂H in MeCN, 95/5 to 50/50 to give [(2S)-1-{[(4-{2-[(2S)-2-(2-{2-[6-(2,5-dioxo-2,5-dihydro-1H-pyrrol-1-yl)hexanamido]acetamido}acetamido)-3-phenylpropanamido]acetamido}phenyl)methoxy]carbonyl}276yrrolidine-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP24) (8.8 mg).

LC-MS (ESI): 768.2 [1/2M+H]⁺.

### 27. Synthesis of LP30

The synthesis of LP30 is shown below:

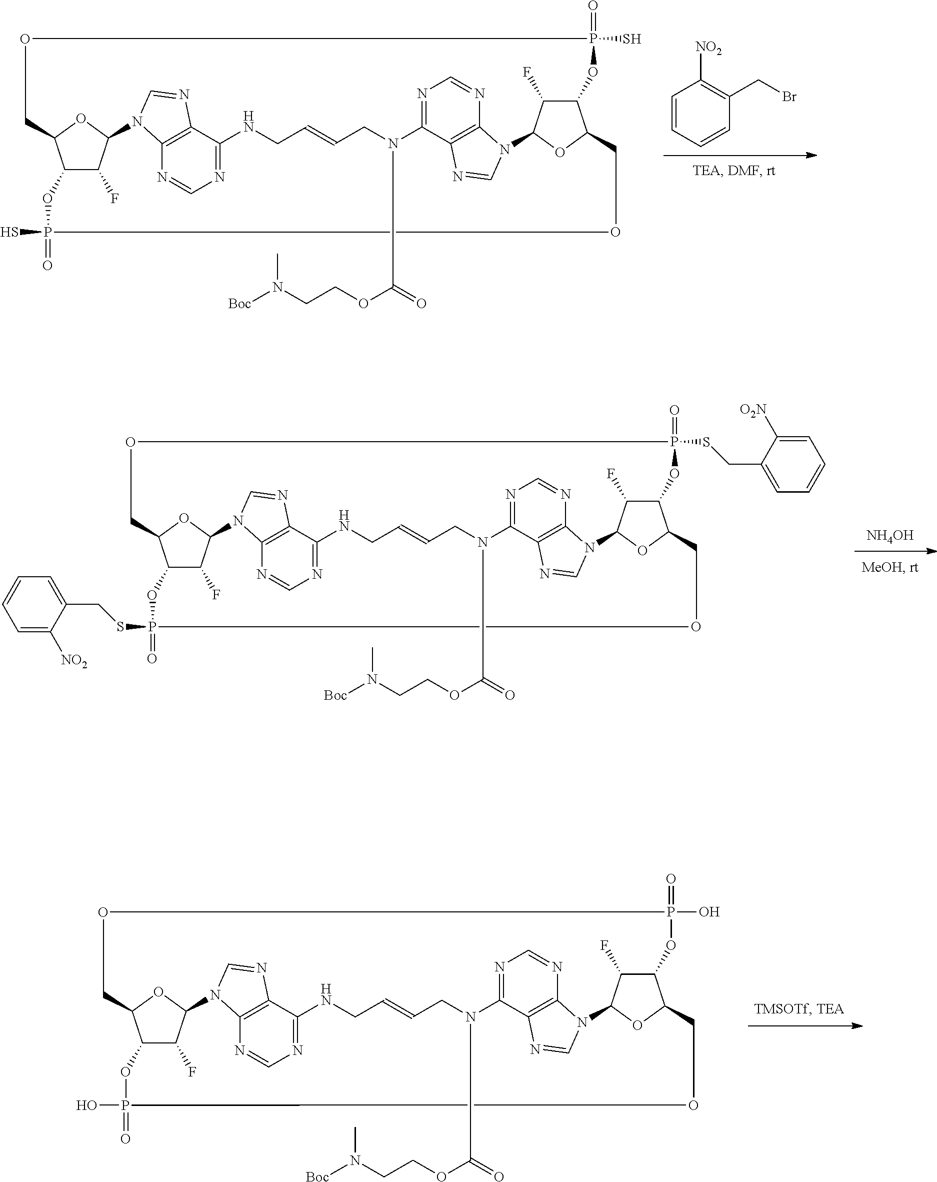

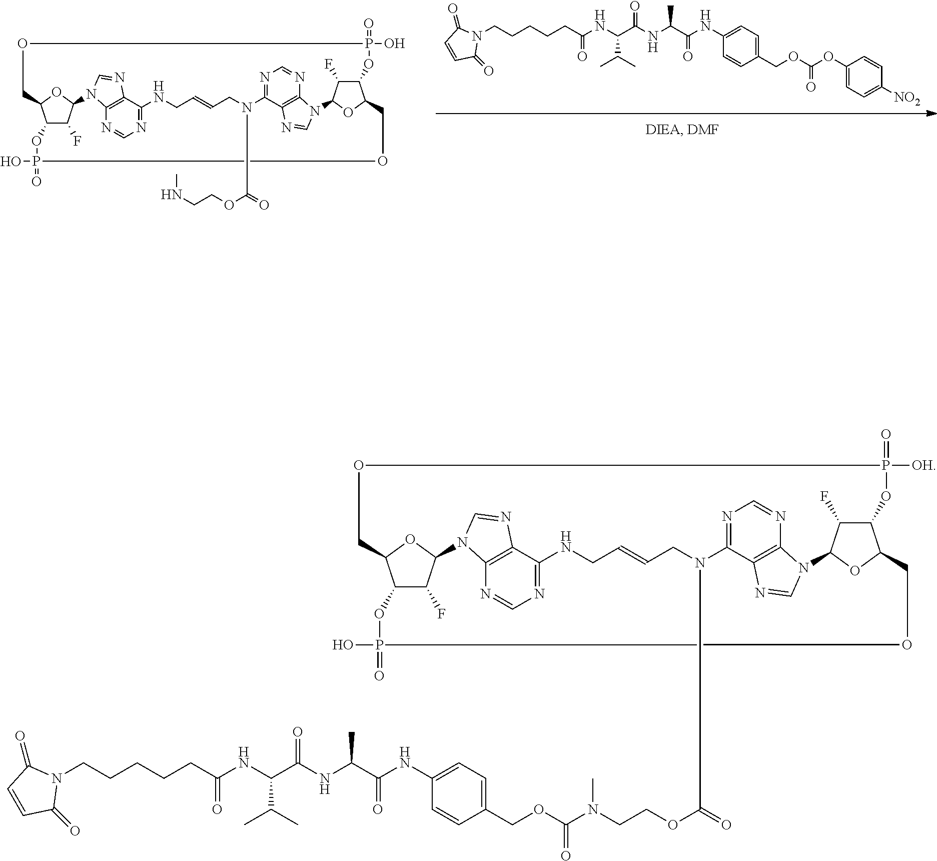 [SMILES: CNCCOC(=O)N1C/C=C/CNc2ncnc3c2ncn3[C@@H]2O[C@@H]3COP(=O)(O)O[C@H]4[C@@H](F)[C@@H](O[C@@H]4COP(=O)(O)O[C@H]3[C@H]2F)n2cnc3c1ncnc32 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4COP(=O)(O)O[C@H]5[C@@H](F)[C@@H](O[C@@H]5COP(=O)(O)O[C@H]4[C@H]3F)n3cnc4c2ncnc43)cc1]

### Synthesis of 2-{[(tert-butoxy)carbonyl](methyl)amino}ethyl(1R,3R,15E,28R,29R,30R,31R,34S,36R,39R,41R)-29,41-difluoro-34,39-bis({[(2-nitrophenyl)methyl]sulfanyl})-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

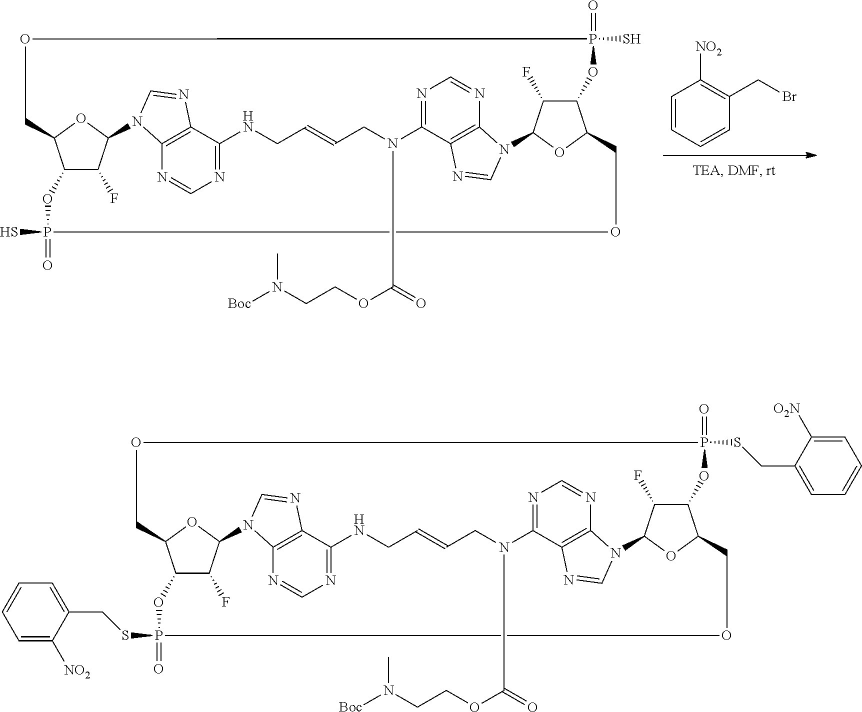 [SMILES: CN(CCOC(=O)N1C/C=C/CNc2ncnc3c2ncn3[C@@H]2O[C@@H]3CO[P@](=O)(S)O[C@H]4[C@@H](F)[C@@H](O[C@@H]4CO[P@@](=O)(S)O[C@H]3[C@H]2F)n2cnc3c1ncnc32)C(=O)OC(C)(C)C ; O=[N+]([O-])c1ccccc1CBr ; CN(CCOC(=O)N1C/C=C/CNc2ncnc3c2ncn3[C@@H]2O[C@@H]3CO[P@](=O)(SCc4ccccc4[N+](=O)[O-])O[C@H]4[C@@H](F)[C@@H](O[C@@H]4CO[P@@](=O)(SCc4ccccc4[N+](=O)[O-])O[C@H]3[C@H]2F)n2cnc3c1ncnc32)C(=O)OC(C)(C)C]

TEA (32.03 mg, 0.315 mmol) was added to a stirred solution of 2-{[(tert-butoxy)carbonyl](methyl)amino}ethyl(1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (60 mg, 0.063 mmol) and 1-(bromomethyl)-2-nitrobenzene (34.19 mg, 0.158 mmol) in DMF (2 mL) at 25° C. The resulting mixture was stirred at 25° C. for 3 h under nitrogen atmosphere. The resulting mixture was concentrated under reduced pressure. The residue was purified by Prep-TLC (CH₂Cl₂ (1% TEA)/MeOH (8/1)) to afford 2-{[(tert-butoxy)carbonyl](methyl)amino}ethyl(1R,3R,15E,28R,29R,30R,31R,345,36R,39R,41R)-29,41-difluoro-34,39-bis({[(2-nitrophenyl)methyl]sulfanyl})-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (70 mg) as a white solid. LC-MS (ESI): 1218.2 [M+H]⁺.

### Synthesis of 2-{[(tert-butoxy)carbonyl](methyl)amino}ethyl(1R,3R,15E,28R,29R,30R,31R,36R,41R)-29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

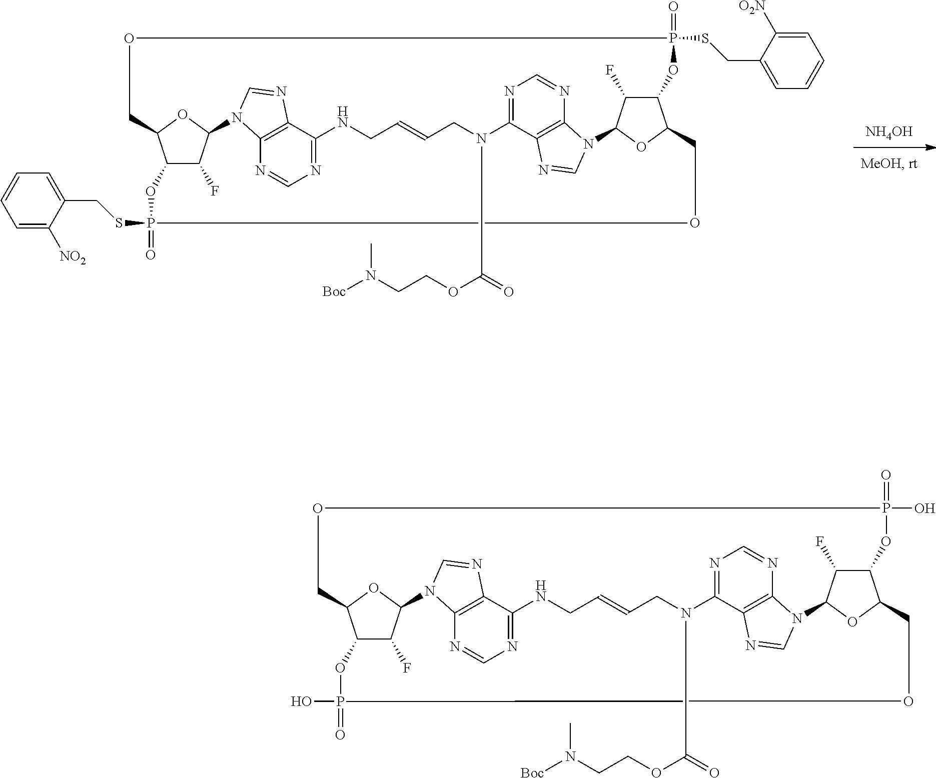 [SMILES: CN(CCOC(=O)N1C/C=C/CNc2ncnc3c2ncn3[C@@H]2O[C@@H]3CO[P@](=O)(SCc4ccccc4[N+](=O)[O-])O[C@H]4[C@@H](F)[C@@H](O[C@@H]4CO[P@@](=O)(SCc4ccccc4[N+](=O)[O-])O[C@H]3[C@H]2F)n2cnc3c1ncnc32)C(=O)OC(C)(C)C ; "NH4OH" ; CN(CCOC(=O)N1C/C=C/CNc2ncnc3c2ncn3[C@@H]2O[C@@H]3COP(=O)(O)O[C@H]4[C@@H](F)[C@@H](O[C@@H]4COP(=O)(O)O[C@H]3[C@H]2F)n2cnc3c1ncnc32)C(=O)OC(C)(C)C]

NH₄OH (2 mL) was added to a stirred mixture of 2-[(tert-butoxycarbonyl)(methyl)amino]ethyl (1R,3R,15E,28R,29R,30R,31R,34S,36R,39R,41R) -29,41-difluoro-34,39-bis({[(2-nitrophenyl)methyl]sulfanyl})-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (70 mg, 0.057 mmol) in MeOH (2 mL) at room temperature. The resulting mixture was stirred at 25° C. for 6 h under nitrogen atmosphere. The resulting mixture was concentrated under reduced pressure. The residue was purified by Prep-TLC (CH₂Cl₂ (1% TEA)/MeOH (6/1)) to afford 2-{[(tert-butoxy)carbonyl](methyl)amino}ethyl(1R,3R,15E,28R,29R,30R,31R,36R,41R)-29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg) as a white solid. LC-MS (ESI): 916.2 [M+H]⁺.

### Synthesis of 2-(methylamino)ethyl (1R,3R,15E,28R,29R,30R,31R,36R,41R) -29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

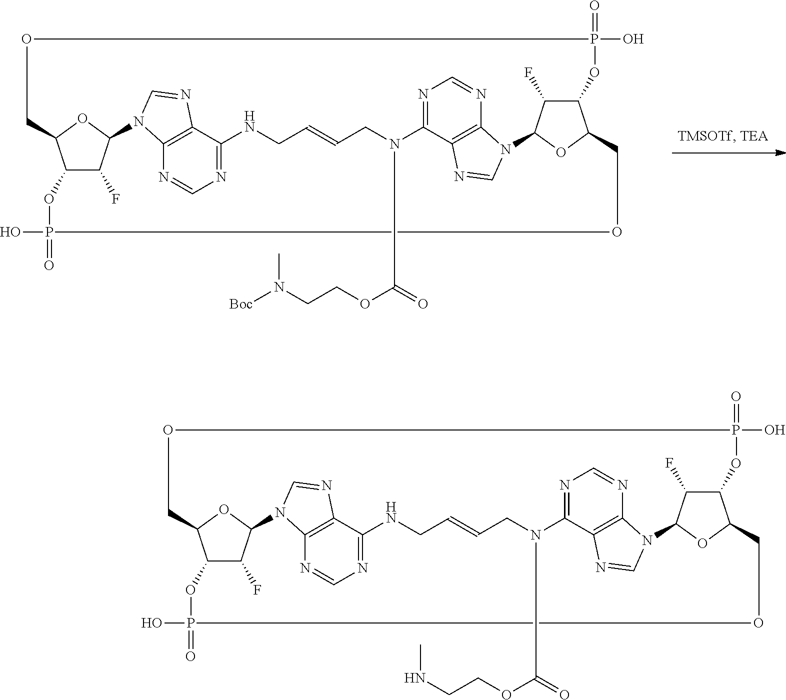 [SMILES: CN(CCOC(=O)N1C/C=C/CNc2ncnc3c2ncn3[C@@H]2O[C@@H]3COP(=O)(O)O[C@H]4[C@@H](F)[C@@H](O[C@@H]4COP(=O)(O)O[C@H]3[C@H]2F)n2cnc3c1ncnc32)C(=O)OC(C)(C)C ; CNCCOC(=O)N1C/C=C/CNc2ncnc3c2ncn3[C@@H]2O[C@@H]3COP(=O)(O)O[C@H]4[C@@H](F)[C@@H](O[C@@H]4COP(=O)(O)O[C@H]3[C@H]2F)n2cnc3c1ncnc32]

TEA (22.10 mg, 0.220 mmol) was added to a stirred solution of 2-[(tert-butoxycarbonyl)(methyl)amino]ethyl (1R,3R,15E,28R,29R,30R,31R,36R,41R) -29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (40 mg, 0.044 mmol) in DCM (2 mL) at 25° C. Then TMSOTf (67.96 mg, 0.308 mmol) was added. The resulting mixture was stirred at 25° C. for 30 min.

The resulting mixture was concentrated under reduced pressure to obtain the crude product of 2-(methylamino)ethyl (1R,3R,15E,28R,29R,30R,31R,36R,41R) -29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate as a colorless oil. LC-MS (ESI): 816.1 [M+H]⁺.

### Synthesis of 2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}ethyl (1R,3R,15E,28R,29R,30R,31R,36R,41R)-29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP30)

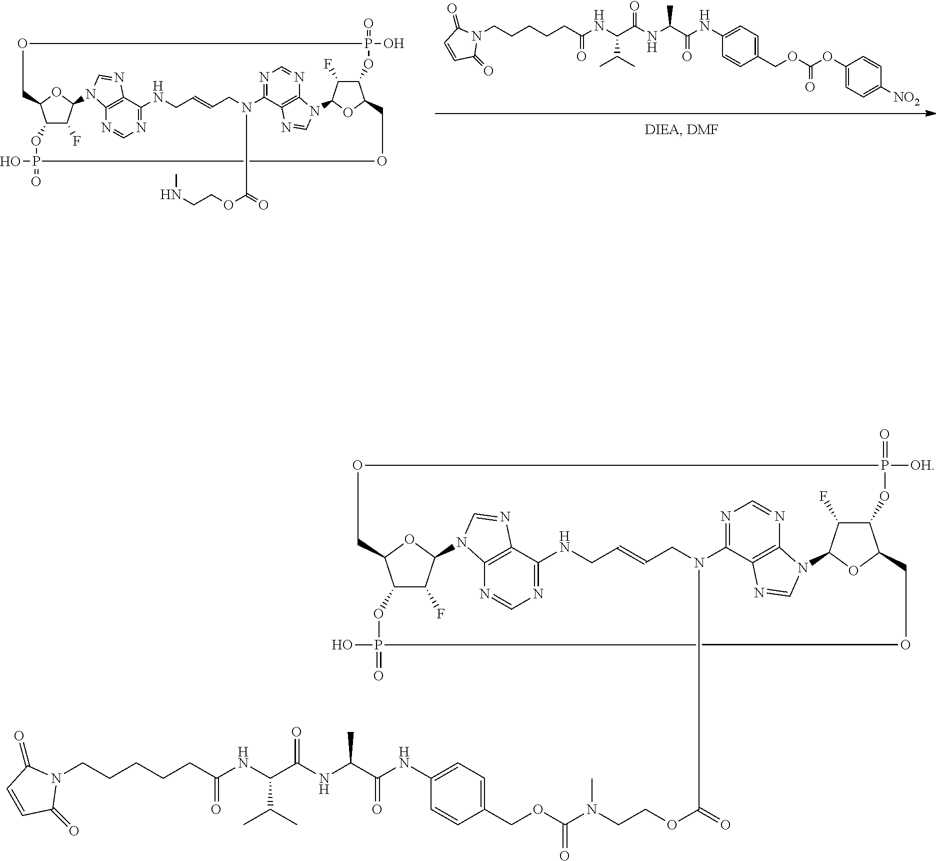 [SMILES: CNCCOC(=O)N1C/C=C/CNc2ncnc3c2ncn3[C@@H]2O[C@@H]3COP(=O)(O)O[C@H]4[C@@H](F)[C@@H](O[C@@H]4COP(=O)(O)O[C@H]3[C@H]2F)n2cnc3c1ncnc32 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](C)C(=O)Nc1ccc(COC(=O)N(C)CCOC(=O)N2C/C=C/CNc3ncnc4c3ncn4[C@@H]3O[C@@H]4COP(=O)(O)O[C@H]5[C@@H](F)[C@@H](O[C@@H]5COP(=O)(O)O[C@H]4[C@H]3F)n3cnc4c2ncnc43)cc1]

DIEA (29.75 mg, 0.230 mmol) was added to a mixture of 2-(methylamino)ethyl (1R,3R,15E,28R,29R,30R,31R,36R,41R) -29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (37.55 mg, 0.046 mmol) and {4-[(2S)-2-[(2S)-2-[6-(2,5-dioxopyrrol-1-yl)hexanamido]-3-methylbutanamido]propanamido]phenyl}methyl 4-nitrophenyl carbonate (30 mg, 0.046 mmol) in DMF (2 mL) at room temperature. The mixture was stirred at room temperature for 14 h under nitrogen atmosphere. The resulting mixture was concentrated under reduced pressure. The crude mixture was purified by Prep-HPLC with the following conditions (Column: XBridge Prep Phenyl OBD Column, 19*250 mm, 5 μm; Mobile Phase A: Water(50 mmol HCO₂NH₄), Mobile Phase B: MeCN; Flow rate: 25 mL/min; Gradient: 20% B to 30% B in 10 min, 30% B; Wavelength: 254 nm) to afford 2-{[({4-[(2S)-2-[(2S)-2-[6-(2,5-dioxo-2,5-dihydro-1H-pyrrol-1-yl)hexanamido]-3-methylbutanamido]propanamido]phenyl}methoxy)carbonyl](methyl)amino}ethyl (1R,3R,15E,28R,29R,30R,31R,36R,41R)-29,41-difluoro-34,39-dihydroxy-34,39-dioxo-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP30) (12.3 mg) as a white solid. LC-MS (ESI): 1328.40 [M+H]⁺. ¹H NMR (400 MHz, Methanol-d₄) δ 8.88-8.69 (m, 2H), 8.50-8.44 (m, 1H), 8.29-8.23 (m, 1H), 8.15-8.09 (m, 1H), 7.91-7.85 (m, 1H), 7.61-7.55 (m, 2H), 7.33-7.20 (m, 2H), 6.82-6.76 (m, 2H), 6.52-6.42 (m, 1H), 6.36-6.30 (m, 1H), 5.66-5.38 (m, 5H), 5.02-4.96 (m, 1H), 4.63-4.57 (m, 5H), 4.56-4.47 (m, 2H), 4.47-4.42 (m, 1H), 4.43-4.28 (m, 2H), 4.23-4.04 (m, 2H), 3.68-3.62 (m, 1H), 3.53-3.42 (m, 3H), 2.81-2.68 (m, 3H), 2.35-2.24 (m, 2H), 2.17-2.02 (m, 1H), 1.73-1.51 (m, 4H), 1.51-1.40 (m, 3H), 1.41-1.23 (m, 2H), 1.04-0.94 (m, 6H).

### 28. Synthesis of LP21

The synthesis of LP21 is shown below:

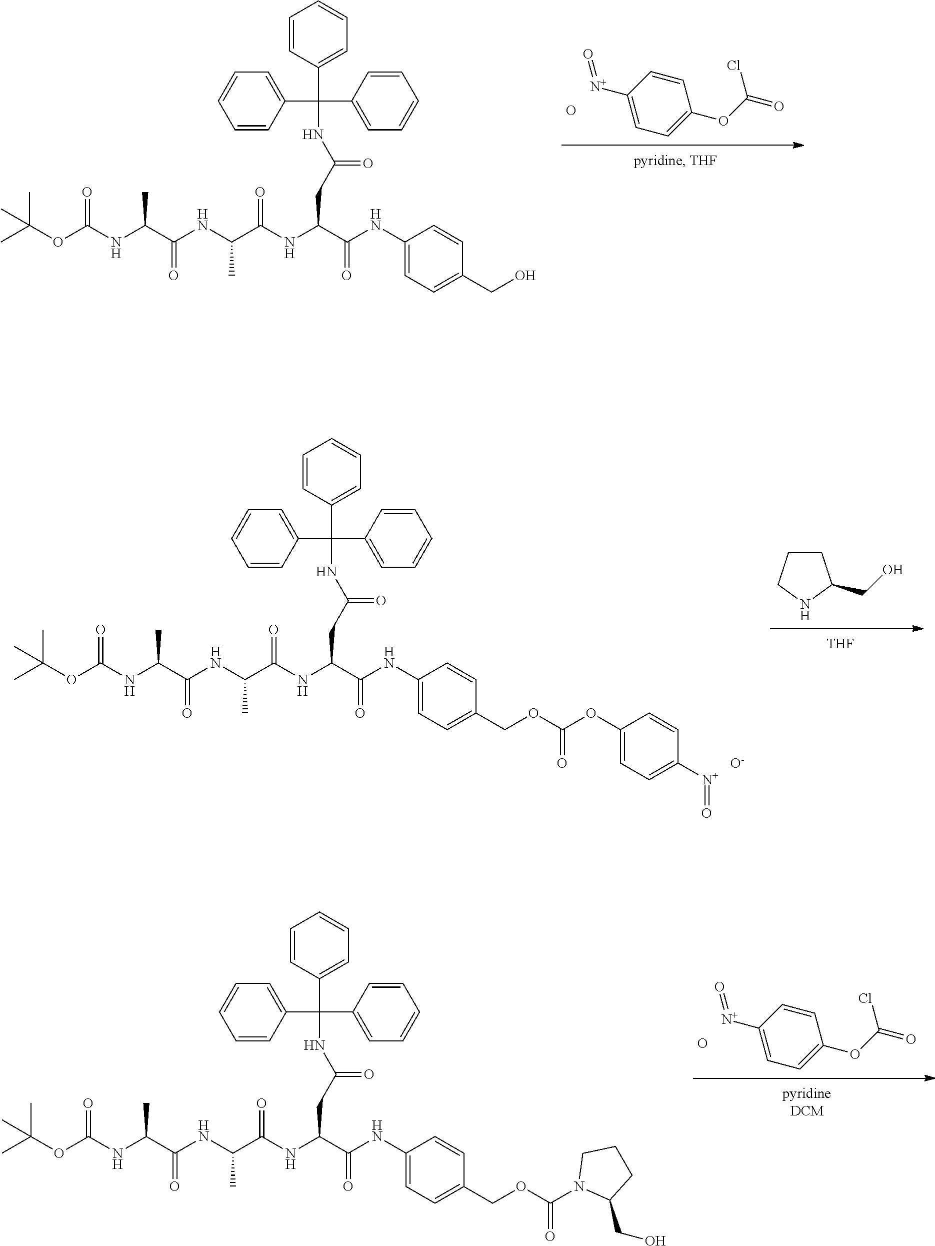

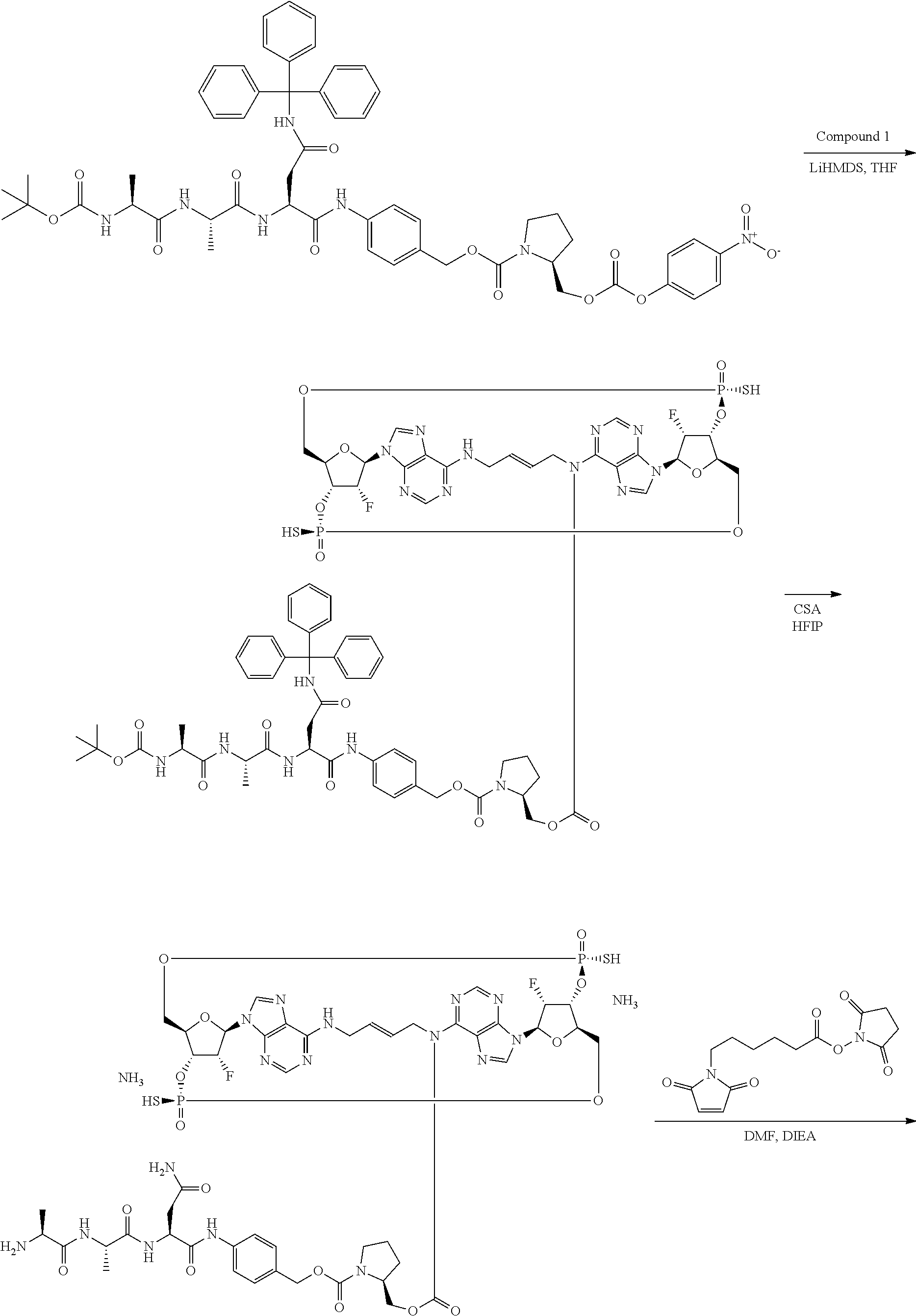

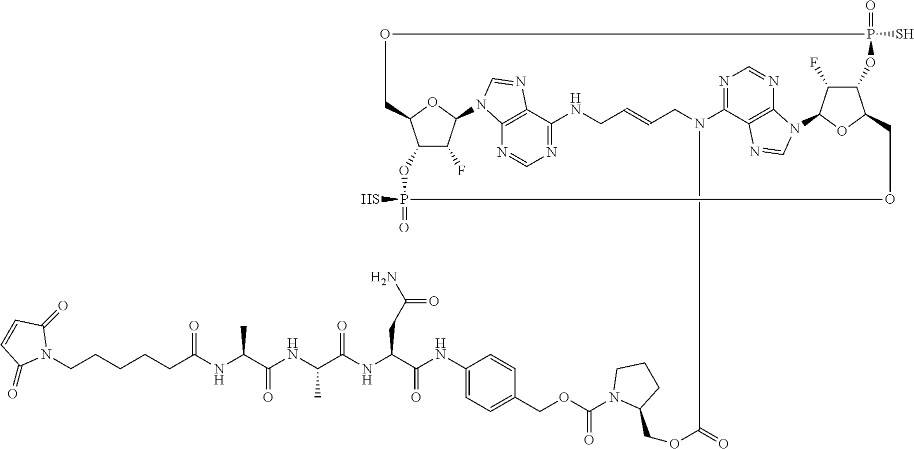 [SMILES: C[C@H](NC(=O)CCCCCN1C(=O)C=CC1=O)C(=O)N[C@@H](C)C(=O)N[C@@H](CC(N)=O)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

### Synthesis of {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl 4-nitrophenyl carbonate

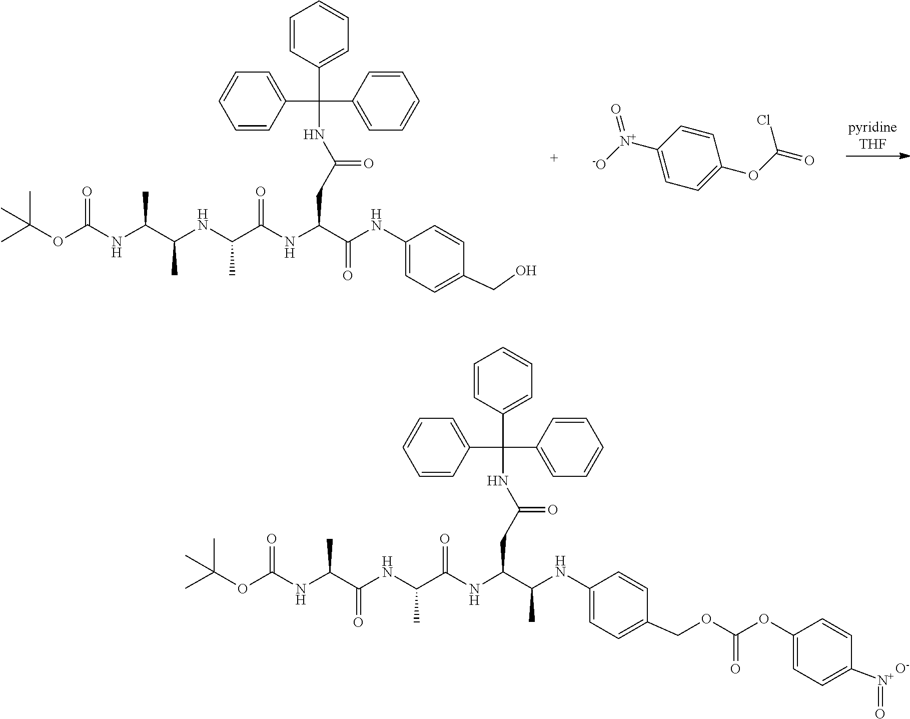

To a solution of tert-butyl N-[(1S)-1-{[(1S)-1-{[(1S)-1-{[4-(hydroxymethyl)phenyl]carbamoyl}-2-[(triphenyl methyl)carbamoyl]ethyl]carbamoyl}ethyl]carbamoyl}ethyl]carbamate (205 mg, 0.284 mmol, CAS 2461517-95-1, prepared by the method in EP4108675) in THF (3.0 mL) were added pyridine (0.046 mL, 0.568 mmol) and 4-nitrophenyl chloroformate (114 mg, 0.568 mmol). After stirring for 2 h at room temperature, additional pyridine (11 μL) and 4-nitrophenyl chloroformate (29 mg) were added at room temperature. After stirring for 45 min at room temperature, ethyl acetate (10 mL) and 10% aqueous citric acid (5 mL) were added to the reaction mixture. The layers were separated. The organic layer was washed with brine (twice), dried over sodium sulfate, filtered, and concentrated. The residue was purified by silica gel column chromatography (EtOAc-heptane=60:40 to 100:0) to give {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl 4-nitrophenyl carbonate (173 mg) as a white solid. LC-MS (ESI): 887.5 [M+H]⁺.

### Synthesis of {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl (2S)-2-(hydroxymethyl)pyrrolidine-1-carboxylate

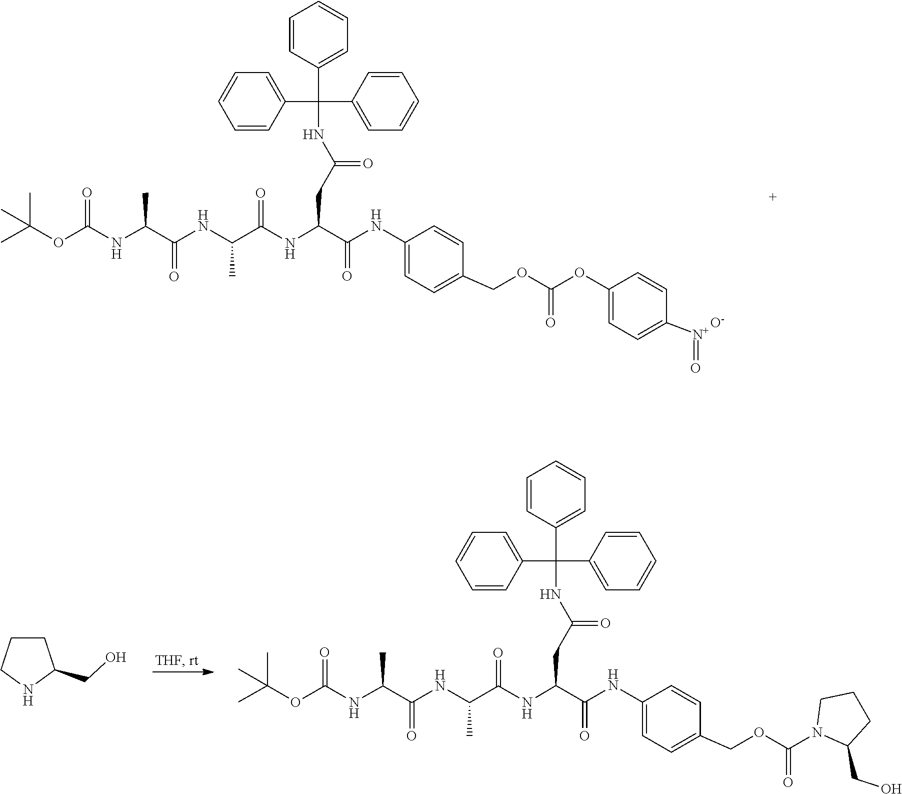

To a solution of {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl 4-nitrophenyl carbonate (173 mg, 0.195 mmol) in tetrahydrofuran (2.0 mL) at room temperature was added L-prolinol (61.4 mg, 0.607 mmol) in THF (0.4 mL). The reaction mixture was stirred at room temperature for 40 mi. Solvent was removed by evaporation. The residue was purified by silica gel column chromatography (EtOAc-heptane=90:10 to 100:0, then EtOAc-MeOH=90:10 to 80:20) to give {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl (2S)-2-(hydroxymethyl)pyrrolidine-1-carboxylate (165 mg) as a colorless oil. LC-MS (ESI): 849.4 [M+H]⁺.

### Synthesis of {4-[(2S)-2-[(2S)-2-[(2S)-2-{[(tert-butoxy)carbonyl]amino)propanamido]propanamido]-3-[(triphenylmethyl)carbamoyl]propanamido]phenyl}methyl (2S)-2-({[(4-nitrophenoxy)carbonyl]oxy}methyl)pyrrolidine-1-carboxylate

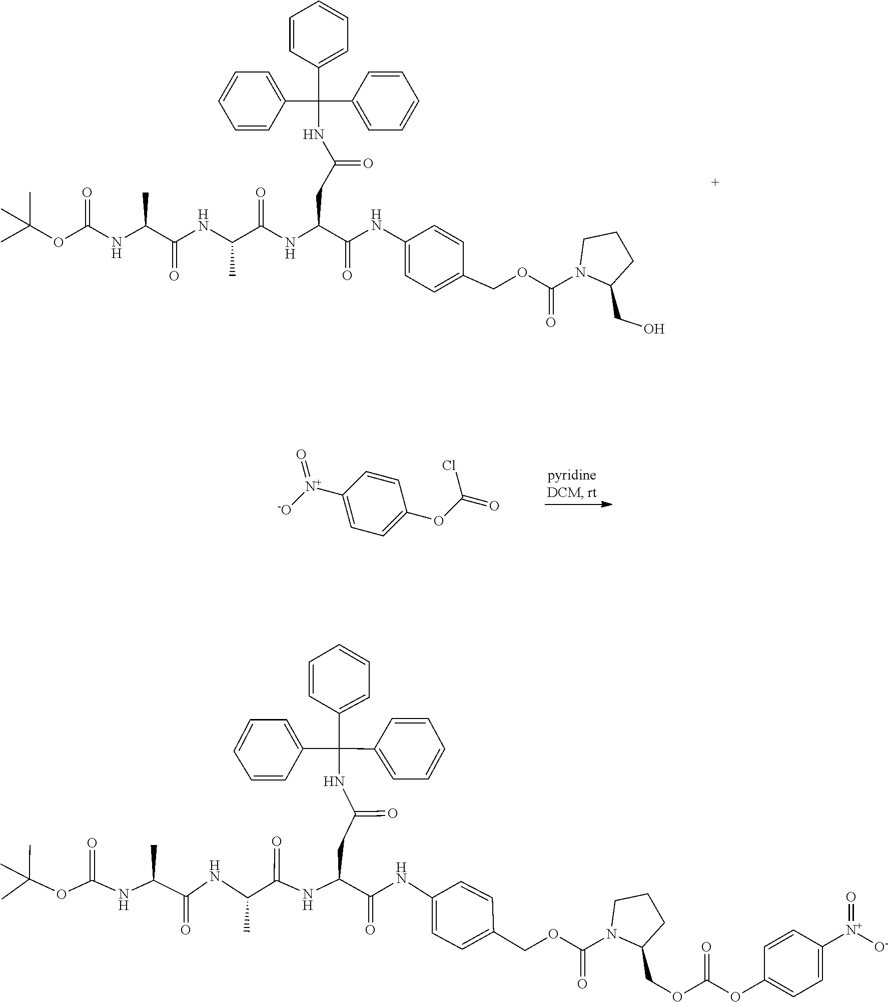

To a solution of {4-[(2S)-2-[(2S)-2-[(2S)-2-{[(tert-butoxy)carbonyl]amino}propanamido]propanamido]-3-[(triphenyl methyl)carbamoyl]propanamido]phenyl})methyl (2S)-2-(hydroxymethyl) pyrrolidine-1-carboxylate (165 mg, 0.194 mmol) in DCM (4.0 mL) at room temperature were added pyridine (31.4 μL, 0.388 mmol) and 4-nitrophenyl chloroformate (78.7 mg, 0.39 mmol). The mixture was stirred at room temperature for 1.5 h. Ethyl acetate (15 mL) and:10% aqueous citric acid (5 mL) were added to the reaction mixture and organic layer was separated, washed with brine (twice), dried over sodium sulfate, and concentrated under reduced pressure. The residue was purified by silica gel column chromatography (EtOAc-heptane=70:30 to 100:0) to give {4-[(2S)-2-[(2S)-2-[(2S)-2-{[(tert-butoxy)carbonyl]amino}propanamido]propanamido]-3-[(triphenylmethyl)carbamoyl]propanamido]phenyl}methyl (2S)-2-({[(4-nitrophenoxy)carbonyl]oxy}methyl)pyrrolidine-1-carboxylate (183 mg) as a colorless oil. LC-MS (ESI): 1014.6 [M+H]⁺.

### Synthesis of [(2S)-1-[({4-[(2S)-2-[(2S)-2-[(2S)-2-{[(tert-butoxy)carbonyl]amino}propanamido]propanamido]-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

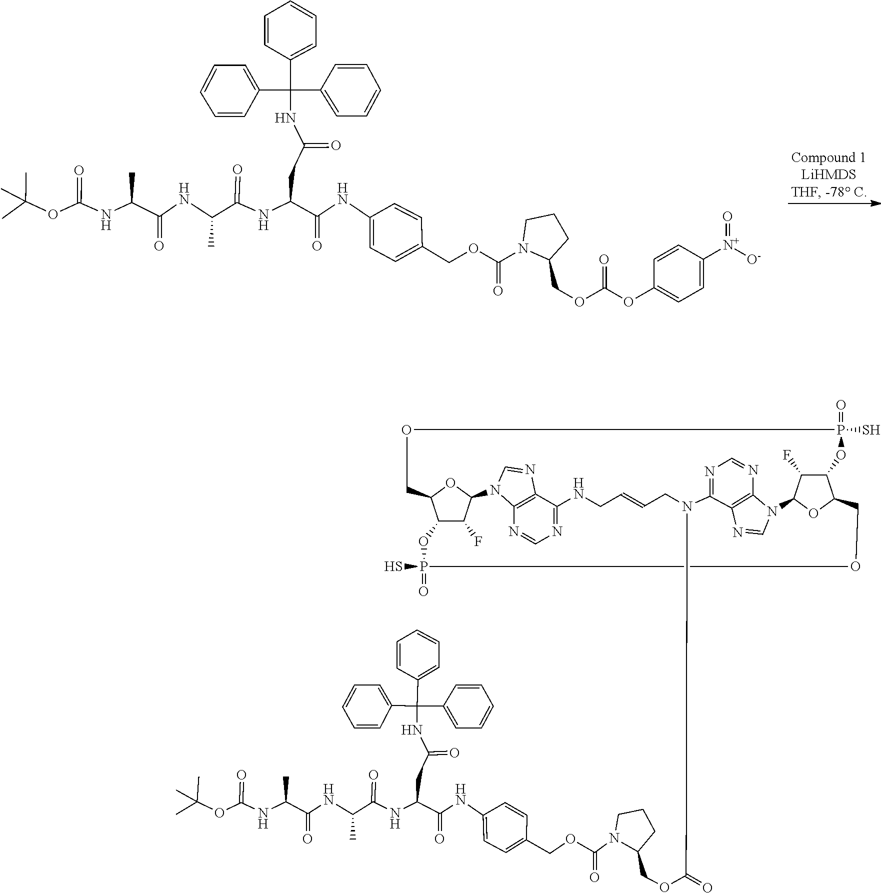 [SMILES: C[C@H](NC(=O)OC(C)(C)C)C(=O)N[C@@H](C)C(=O)N[C@@H](CC(=O)NC(c1ccccc1)(c1ccccc1)c1ccccc1)C(=O)Nc1ccc(COC(=O)N2CCC[C@H]2COC(=O)Oc2ccc([N+](=O)[O-])cc2)cc1 ; C[C@H](NC(=O)OC(C)(C)C)C(=O)N[C@@H](C)C(=O)N[C@@H](CC(=O)NC(c1ccccc1)(c1ccccc1)c1ccccc1)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

To a white suspension of Compound 1 (51.6 mg, 0.066 mmol) in tetrahydrofuran (5.0 mL) at room temperature was added LiHMDS (0.51 mL, 0.663 mmol) dropwise for 2 min. The reaction mixture was stirred at room temperature for 40 min. Then, it was cooled to −78° C. To the mixture was added {4-[(2S)-2-[(2S)-2-[(2)-2-{[(tert-butoxy)carbonyl]amino}propanamido]propanamido]-3-[(triphenylmethyl)carbamoyl]propanamido]phenyl}methyl (2S)-2-({[(4-nitrophenoxy)carbonyl]oxy}methyl)pyrrolidine-1-carboxylate (67.7 mg, 0.067 mmol) in THF (0.5 mL) with THF rinse (0.25 mL×2). The reaction mixture was stirred at −78° C. for 15 min. The cooling bath was removed, and the reaction mixture was allowed to warm to room temperature. After 40 min, the reaction mixture was cooled to −70° C. The reaction was quenched with acetic acid (95 μL, 1.652 mmol) at −70° C. Then it was stirred at 4° C. for 1 h. The mixture was concentrated under reduced pressure. The residue was purified by ODS silica gel column chromatography (H₂O-MeCN=90:10 to 30:70 including 0.1% formic acid) to give [(2S)-1-[({4-[(2S)-2-[(2S)-2-[(2S)-2-{[(tert-butoxy)carbonyl]amino}propanamido]propanamido]-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (60.3 mg) as a white solid. LC-MS (ESI): 811.6 1/2[M+2H]²⁺

### Synthesis of [(2S)-1-[({4-[(2S)-2-[(2S)-2-[(2S)-2-aminopropanamido]propanamido]-3-carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate diammonium salt

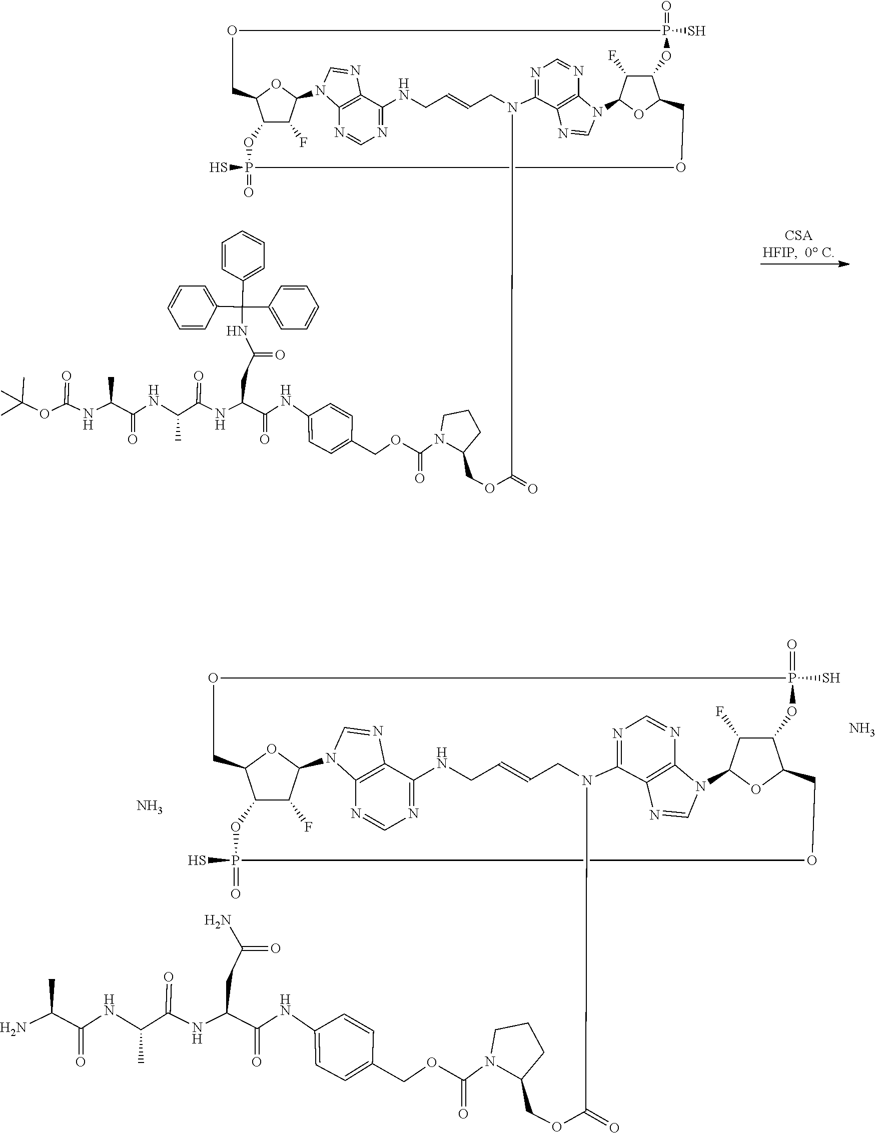

To a solution of [(2S)-1-[({4-[(2S)-2-[(2S)-2-[(2S)-2-{[(tert-butoxy)carbonyl]amino}propanamido]propanamido]-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (30 mg, 0.019 mmol) in 1,1,1,3,3,3-hexafluoro-2-propanol (3.0 mL) at 0° C. was added CSA (86 mg, 0.37 mmol). The reaction mixture was stirred at 0° C. for 3 h. The reaction mixture was quenched with MeOH. The mixture was concentrated under reduced pressure to ca. 1 mL. The residue was purified by ODS silica gel column chromatography (H₂O-MeCN with 0.1% NH₃=95:5 to 55:45) to give [(2S)-1-[({4-[(2S)-2-[(2)-2-[(2)-2-aminopropanamido]propanamido]-3-carbamoylpropanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate diammonium salt (11.8 mg) as a white solid. LC-MS (ESI): 1279.4 [M+H]⁺.

### Synthesis of [(2S)-1-[({4-[(2S)-3-carbamoyl-2-[(2S)-2-[(2S)-2-[6-(2,5-dioxo-2,5-dihydro-1H-pyrrol-1-yl)hexanamido]propanamido]propanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP21)

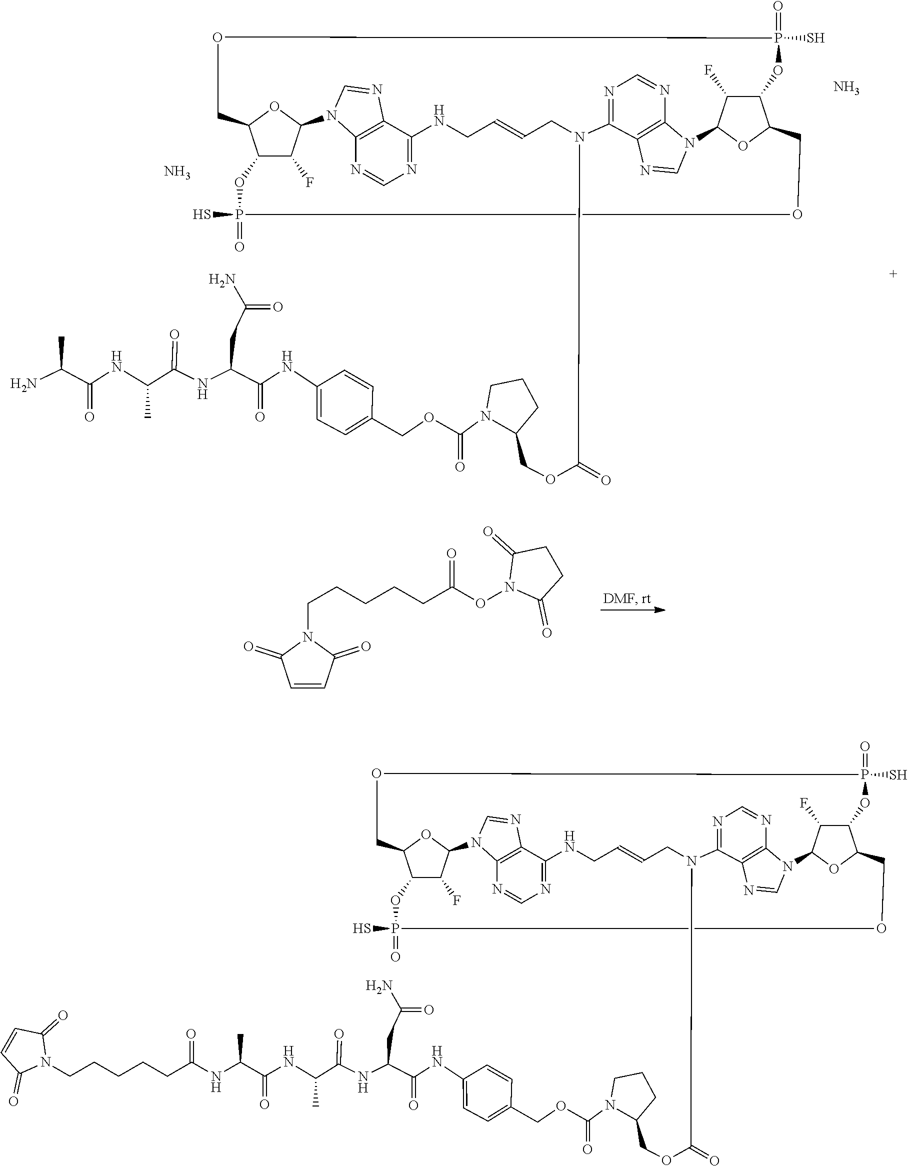

To a solution of [(2S)-1-[({4-[(2S)-2-[(2)-2-[(2)-2-aminopropanamido]propanamido]-3-carbamoylpropanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate diammonium salt (11.8 mg, 8.986 μmol) and N-succinimidyl 6-maleimidocaproate (3.5 mg, 0.011 mmol) in DMF (1000 μL, 12.915 mmol) at room temperature was added DIEA (3.13 μL, 0.018 mmol). The reaction mixture was stirred at room temperature. After stirring for 1 h, additional N-succinimidyl 6-maleimidocaproate (3.7 mg) and DIEA (3.13 μL) were added. After stirring for 2 h, the reaction mixture was directly purified by ODS silica gel column chromatography (H₂O-MeCN=95:5 to 55:45) to give [(2S)-1-[({4-[(2S)-3-carbamoyl-2-[(2S)-2-[(2S)-2-[6-(2,5-dioxo-2,5-dihydro-1H-pyrrol-1-yl)hexanamido]propanamido]propanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34λ⁵,39λ⁵-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (LP21) (6.11 mg) as a solid.

LC-MS (ESI): 737.2 1/2 [M+2H]²⁺.

### 29. Synthesis of LP22

The synthesis of LP22 is shown below:

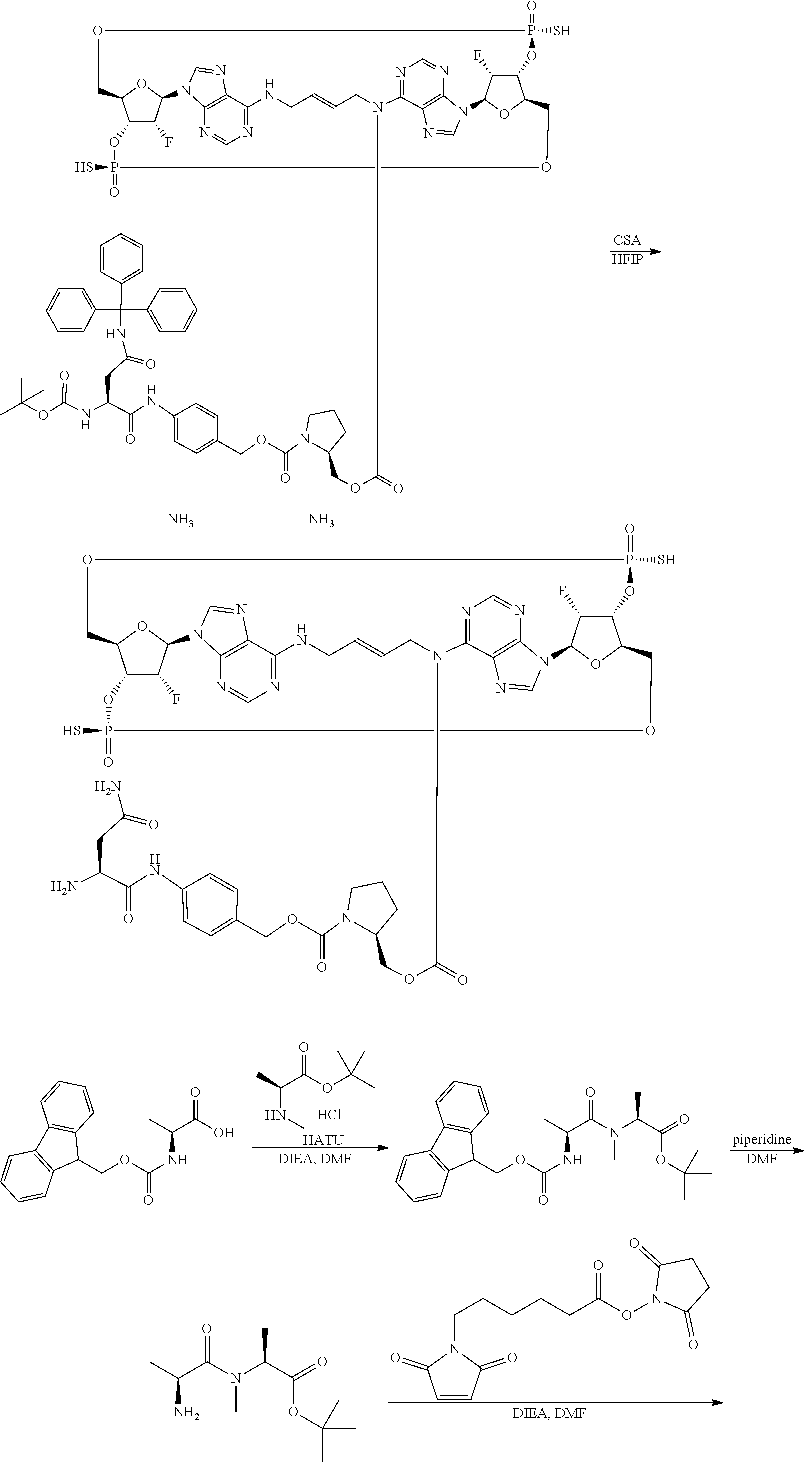

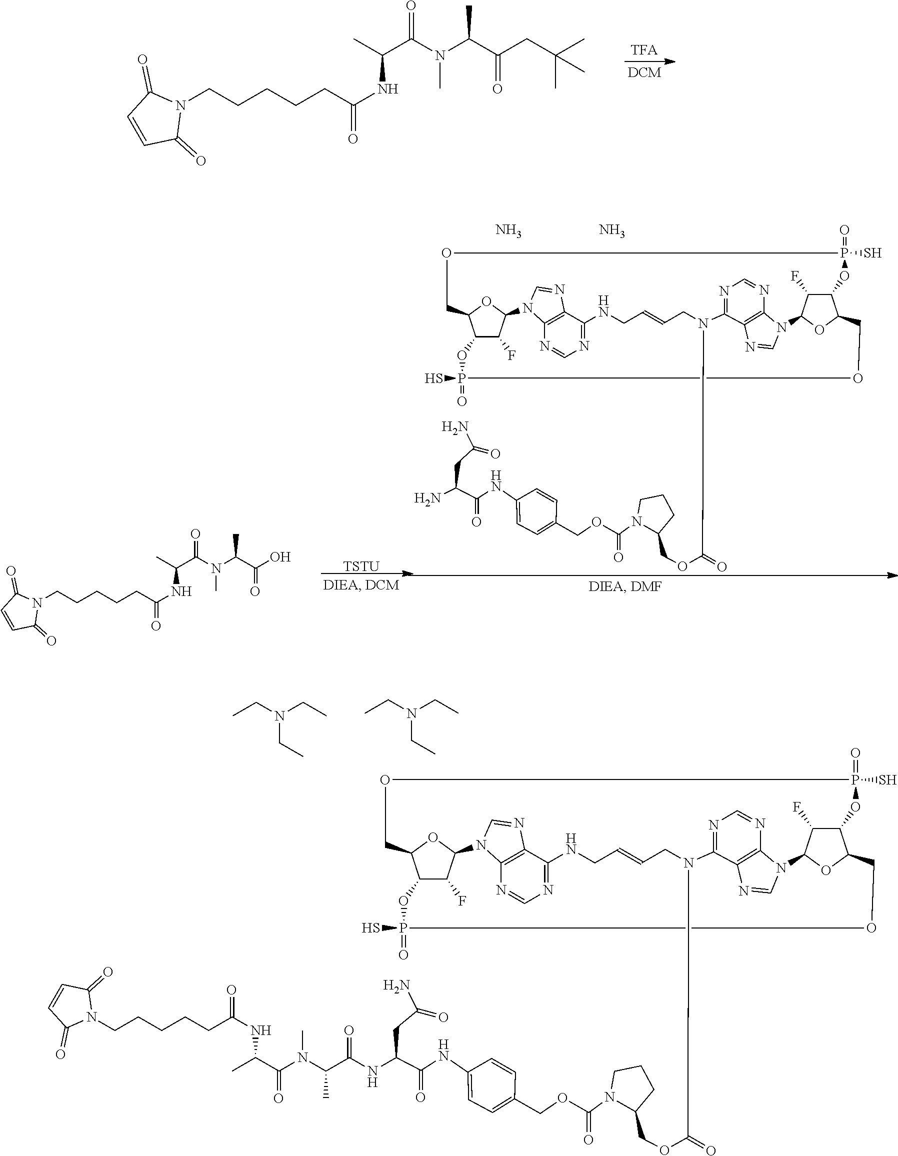

### Synthesis of tert-butyl (2S)-2[(2S)-2-({[(9H-fluoren-9-yl)methoxy]carbonyl}amino)-N-methylpropanamido]propanoate

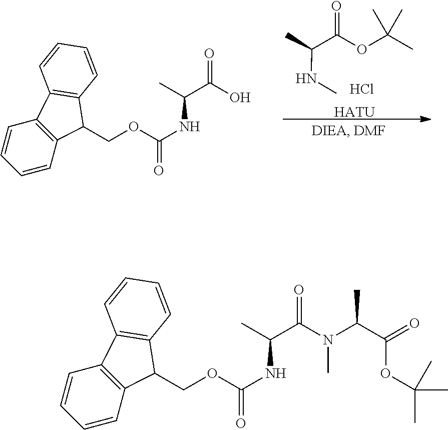

To a solution of (2S)-2-({[(9H-fluroen-9-yl)methoxy]carbonyl}amno)propanoic acid (300 mg, 0.964 mmol) in DMF (8 mL) were added DIEA (0.505 mL, 2.89 mmol) and HATU (440 mg, 1.16 mmol). The mixture was stirred for 10 min. To the mixture was added tert-butyl (2S)-2-(methylamino)propanoate hydrochloride (198 mg, 1.01 mmol). The mixture was stirred or 1 h. The resulting mixture was diluted with EtOAc and water. The organic layer was separated, washed with brine, dried over Na₂SO₄, and concentrated under reduced pressure. The residue was purified by NH silica gel column chromatography (EtOAc/heptane=1/5) to give (2S)-2-[(2S)-2-({[(9H-fluoren-9-yl)methoxy]carbonyl}amino)-N-methylpropanamido]propanoate (399.1 mg). LC-MS (ESI): 453.4 [M+H]⁺.

### Synthesis of tert-butyl (2S)-2-[(2S)-2-amino-N-methylpropanamido]propanoate

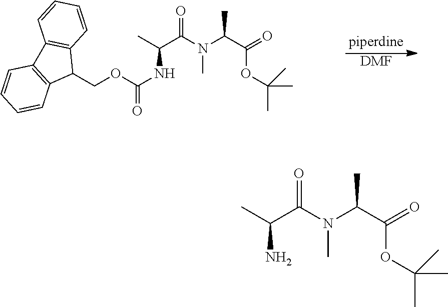

To a solution of (2S)-2-[(2S)-2-({[(9H-fluoren-9-yl)methoxy]carbonyl}amino)-N-methylpropanamido]propanoate (199.1 mg, 0.44 mmol) in DMF (6 mL) at 0° C. was added piperidine (0.436 mL, 4.40 mmol). The mixture was stirred for 2 h at 0° C. The resulting mixture was concentrated under reduced pressure. The residue was purified by ODS silica gel column chromatography (H2O/MeCN including 0.1% formic acid=98/2 to 50/50) to give tert-butyl (2S)-2-[(2S)-2-amino-N-methylpropanamido]propanoate (41.7 mg). LC-MS (ESI): 231.2 [M+H]⁺.

### Synthesis of tert-butyl (2S)-2-[(2S)-2-[6-(2,5-dioxo-2,5-dihydro-1H-pyrrol-1-yl)hexanamido]-N-methylpropanamido]propanoate

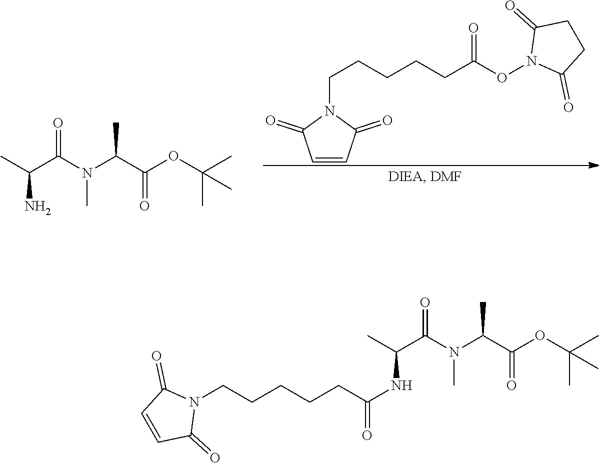 [SMILES: C[C@H](N)C(=O)N(C)[C@@H](C)C(=O)OC(C)(C)C ; O=C(CCCCCN1C(=O)C=CC1=O)ON1C(=O)CCC1=O ; C[C@H](NC(=O)CCCCCN1C(=O)C=CC1=O)C(=O)N(C)[C@@H](C)C(=O)OC(C)(C)C]

To a solution of 2,5-dioxopyrrolidin-1-yl 6-(2,5-dioxo-2,5-dihydro-1H-pyrrol-1-yl)hexanoate (125 mg, 0.405 mmol) and DIEA (0.129 mL, 0.736 mmol) in DMF (3 mL) was added tert-butyl (2S)-2-[(2S)-2-amino-N-methylpropanamido]propanoate (84.8 mg, 0.368 mmol). The mixture was stirred for 30 min. The resulting mixture was concentrated under reduced pressure. The residue was purified by ODS silica gel column chromatography (H₂O/MeCN containing 0.1% formic acid=98/2 to 50/50) to give tert-butyl (2S)-2-[(2S)-2-[6-(2,5-dioxo-2,5-dihydro-1H-pyrrol-1-yl)hexanamido]-N-methylpropanamido]propanoate (108.4 mg). LC-MS (ESI): 424.4 [M+H]⁺.

### Synthesis of (2S)-2-[(2S)-2-[6-(2,5-dioxo-2,5-dihydro-1H-pyrrol-1-yl)hexanamido]-N-methylpropanamido]propanoic acid

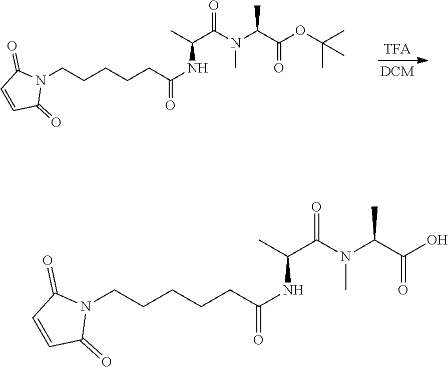 [SMILES: C[C@H](NC(=O)CCCCCN1C(=O)C=CC1=O)C(=O)N(C)[C@@H](C)C(=O)OC(C)(C)C ; C[C@H](NC(=O)CCCCCN1C(=O)C=CC1=O)C(=O)N(C)[C@@H](C)C(=O)O]

To a solution of tert-butyl (2S)-2-[(2S)-2-[6-(2,5-dioxo-2,5-dihydro-1H-pyrrol-1-yl)hexanamido]-N-methylpropanamido]propanoate (108.4 mg) in DCM (2 mL) at 0° C. was added TFA (2 mL, 25.96 mmol). The mixture was stirred for 30 min at 0° C. The mixture was warmed to room temperature and was stirred for 90 min. The resulting mixture was concentrated and azeotroped with toluene under reduced pressure to give (2S)-2-[(2S)-2-[6-(2,5-dioxo-2,5-dihydro-1H-pyrrol-1-yl)hexanamido]-N-methylpropanamido]propanoic acid (94.5 mg). The obtained material was used without further purification. LC-MS (ESI): 368.3 [M+H]⁺.

### Synthesis of [(2S)-1-[({4-[(2S)-2-amino-3-carbamoylpropanamido]phenyl}methoxy)carbonyl]pyrrolidin-2-yl]methyl (1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate diammonium salt

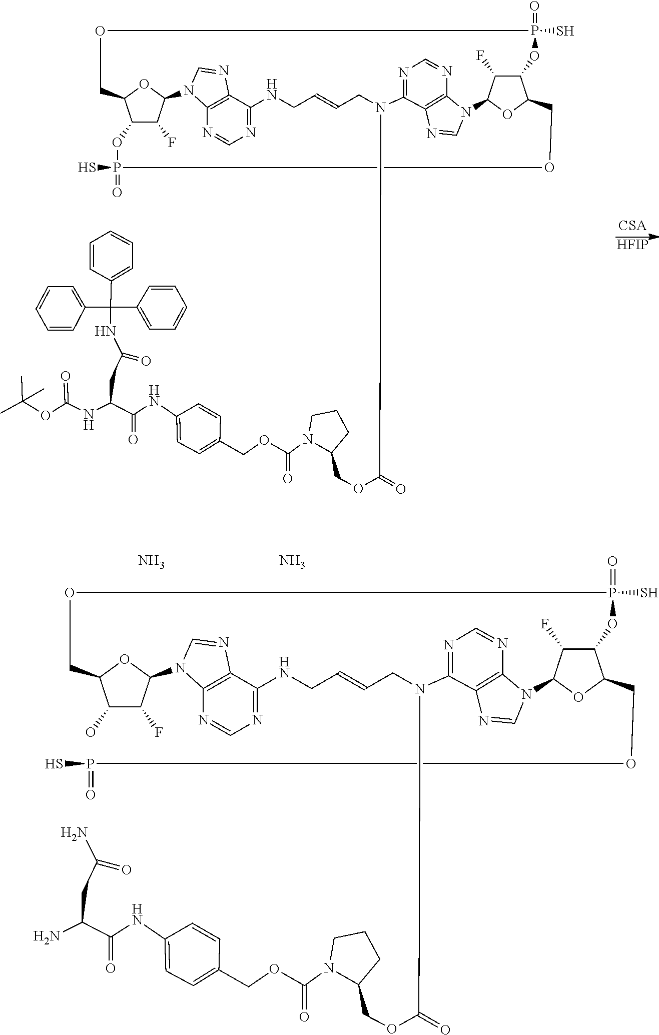

To a solution of [(2S)-1-[({4-[(2S)-2-{[(tert-butoxy)carbonyl]amino}-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (50 mg, 0.034 mmol) in 1,1,1,3,3,3-hexafluoropropan-2-ol (5 mL) at 0° C. was added [(1S,4R)-7,7-dimethyl-2-oxobicyclo[2.2.1]heptan-1-yl]methanesulfonic acid (157 mg, 0.676 mmol). The mixture was stirred for 3 h at 0° C. The reaction mixture was quenched with MeOH. The resulting mixture was concentrated under reduced pressure. The residue was purified by ODS silica gel column chromatography (H₂O/MeCN including 0.1% ammonia=98/2 to 40/60) to give [(25)-1-[({4-[(2S)-2-amino-3-carbamoylpropanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate diammonium salt (16.1 mg).

LC-MS (ESI): 1137.4 [M+H]⁺.

### Synthesis of [(2S)-1-[({4-[(2S)-3-carbamoyl-2-[(2S)-2-[(2S)-2-[6-(2,5-dioxo-2,5-dihydro-1H-pyrrol-1-yl)hexanamido]-N-methylpropanamido]propanamido]propanamido]phenyl}methoxy)carbonyl]pyrrolidin-2-yl]methyl(1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate bis(triethylamine) salt (LP22.2NEt₃)

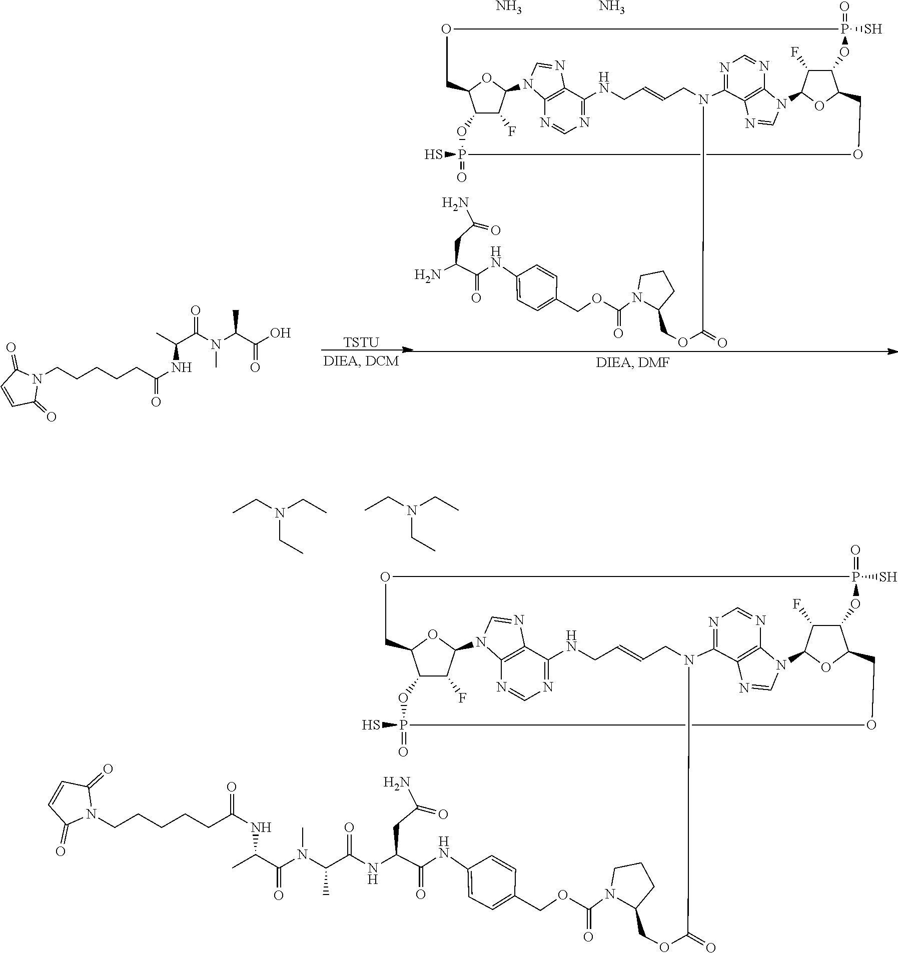

To a solution of (2S)-2-[(2S)-2-[6-(2,5-dioxo-2,5-dihydro-1H-pyrrol-1-yl)hexanamido]-N-methylpropanamido]propanoic acid (16.7 mg, 0.045 mmol) and DIEA (0.048 mL, 0.275 mmol) in DCM (1 mL) was added TSTU (12.4 mg, 0.0.41 mmol). The mixture was stirred for 1 h. To the mixture was added TSTU (6.5 mg). The mixture was stirred for 30 min. The resulting mixture was concentrated under reduced pressure. The residue was diluted with DMF (500 mL) and was added to the solution of [(2S)-1-[({4-[(2S)-2-amino-3-carbamoylpropanamido]phenyl}methoxy)carbonyl]pyrrolidin-2-yl]methyl (1R,3R,15E,28R,29R,30R,31R,34S,36R,39R,41R) -29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylatediamine (16.1 mg, 0.14 mmol) and DIEA (20 mL, 0.115 mmol) in DMF (1 mL). The mixture was stirred for 90 min. The resulting mixture was concentrated under reduced pressure. The residue was purified by ODS silica gel column chromatography (H₂O/MeCN including 0.1% formic acid=98/2 to 40/60) to give crude material. The crude material was purified by Prep-TLC (CHCl₃/MeOH containing 1% TEA=4/1) to give [(2S)-1-[({4-[(2S)-3-carbamoyl-2-[(2S)-2-[(2S)-2-[6-(2,5-dioxo-2,5-dihydro-1H-pyrrol-1-yl)hexanamido]-N-methylpropanamido]propanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate bis(triethylamine)salt (LP22.2NEt₃) (3.40 mg).

LC-MS (ESI): 1486.4 [M+H]⁺.

### 30. Synthesis of LP23

The synthesis of LP23 is shown below:

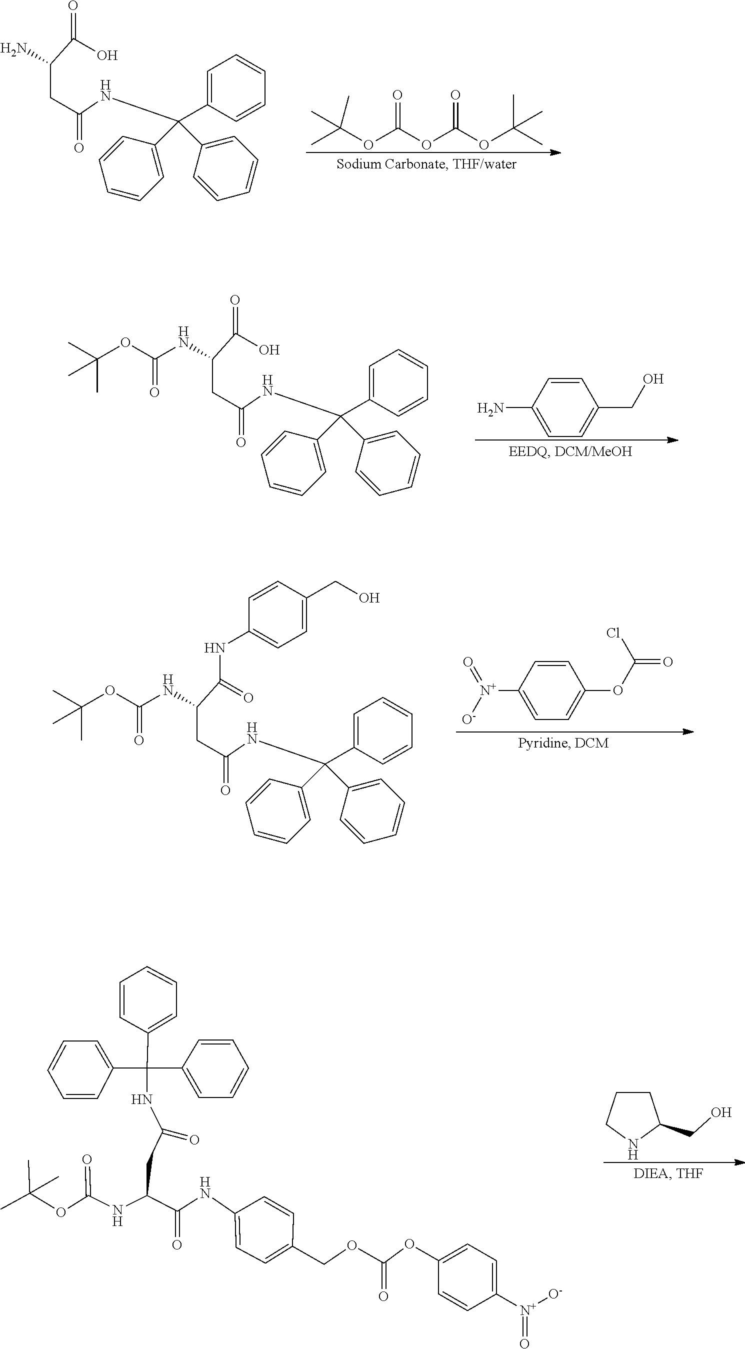

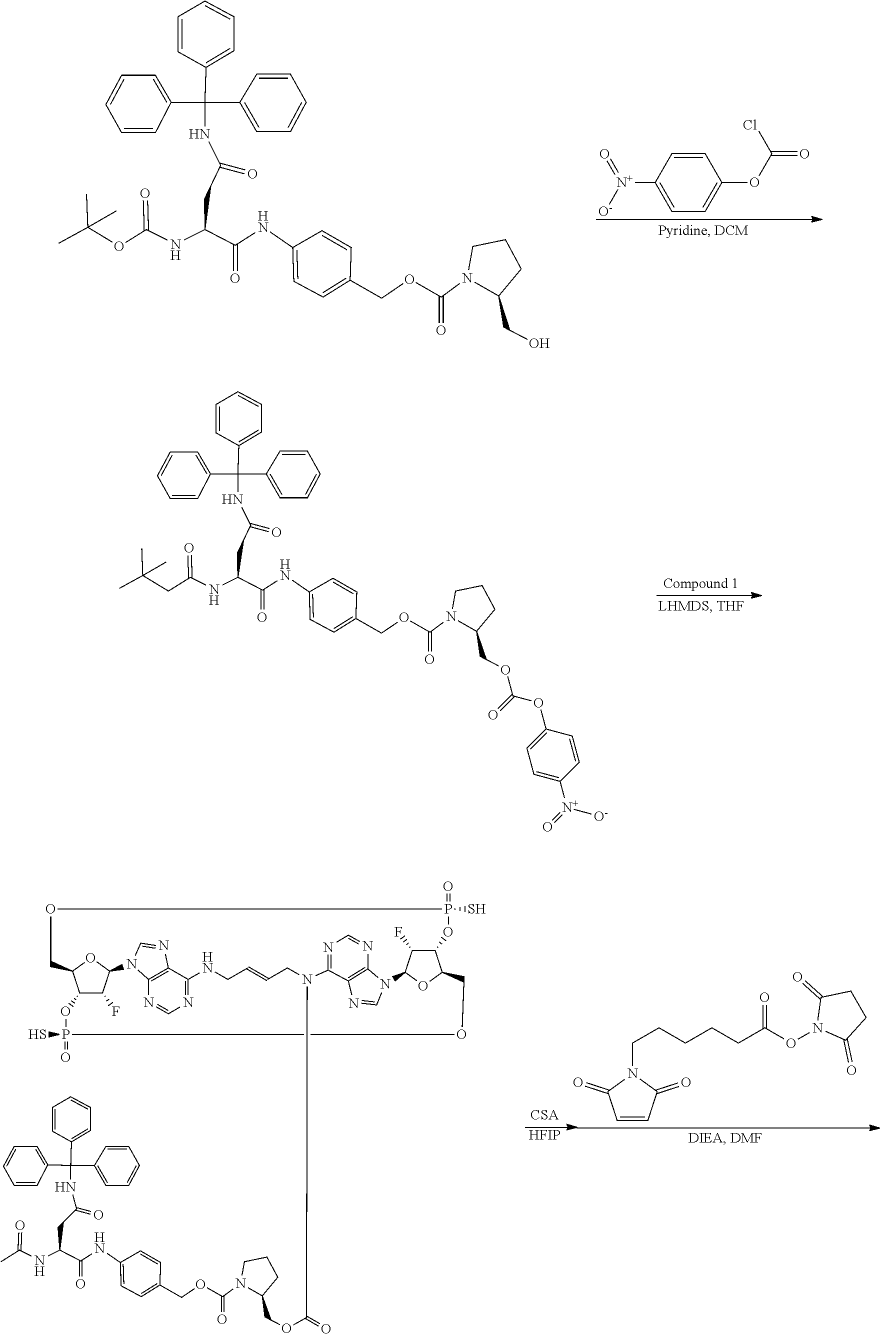

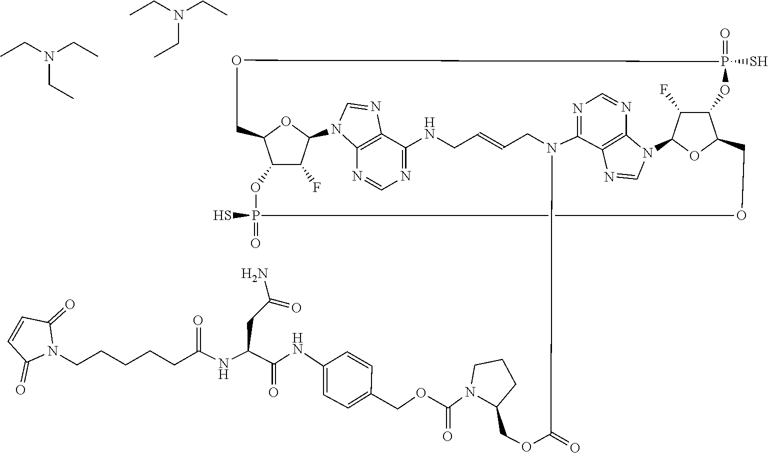

### Synthesis of (2S)-2-{[(tert-butoxy)carbonyl]amino}-3-[(triphenylmethyl)carbamoyl]propanoic acid

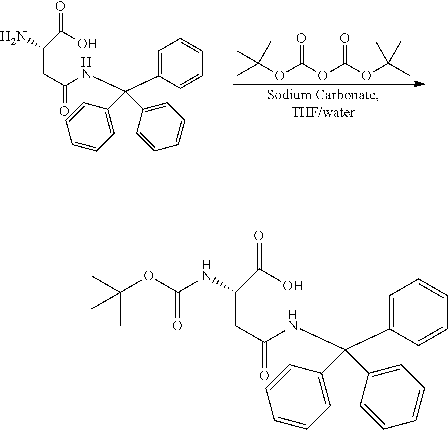

To a solution of (2S)-2-amino-3-[(triphenylmethyl)carbamoyl]propanoic acid (2 g, 5.34 mmol) in THF (14.0 mL) and water (14.0 mL) at 0° C. was added Sodium Carbonate (1.19 g, 11.2 mmol). After 10 min, di-tert-butyl dicarbonate (1.63 g, 7.48 mmol) was added to the mixture. The mixture was stirred at room temperature for 4 h 20 min. 5 N HCl was added to the mixture until pH 4. The aqueous layer was extracted with EtOAc, and the organic layer was concentrated under reduced pressure. The obtained crude (2S)-2-{[(tert-butoxy)carbonyl]amino}-3-[(triphenylmethyl)carbamoyl]propanoic acid (2.53 g) was used to next step without purification. LC-MS (ESI): 475.4 [M+H]⁺.

### Synthesis of tert-butyl N-[(1S)-1-{[4-(hydroxymethyl)phenyl]carbamoyl}-2-[(triphenylmethyl)carbamoyl]ethyl]carbamate

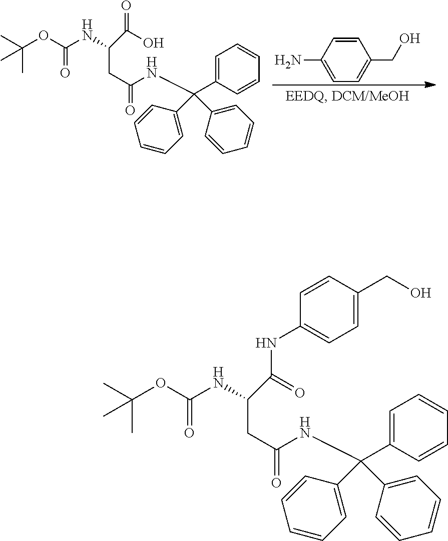

To a solution of (2S)-2-{[(tert-butoxy)carbonyl]amino}-3-[(triphenylmethyl)carbamoyl]propanoic acid (1.3 g, 2.74 mmol) and (4-aminophenyl)methanol (0.371 g, 3.01 mmol) in DCM (30 mL) and MeOH (10 mL) at 0° C. was added Ethyl 2-ethoxy-1,2-dihydroquinoline-1-carboxylate (1.36 g, 5.48 mmol). The solution was stirred at room temperature for 18 h. The resulting mixture was concentrated under reduced pressure. The residue was purified by silica gel column chromatography (EtOAc/heptane=1/1 to 3/1) to give tert-butyl N-[(1S)-1-{[4-(hydroxymethyl)phenyl]carbamoyl}-2-[(triphenylmethyl)carbamoyl]ethyl]carbamate (1.22 g). LC-MS (ESI): 580.4 [M+H]⁺.

### Synthesis of {4-[(2S)-2-{[(tert-butoxy)carbonyl]amino}-3-[(triphenylmethyl)carbamoyl]propanamido]phenyl}methyl 4-nitrophenyl carbonate

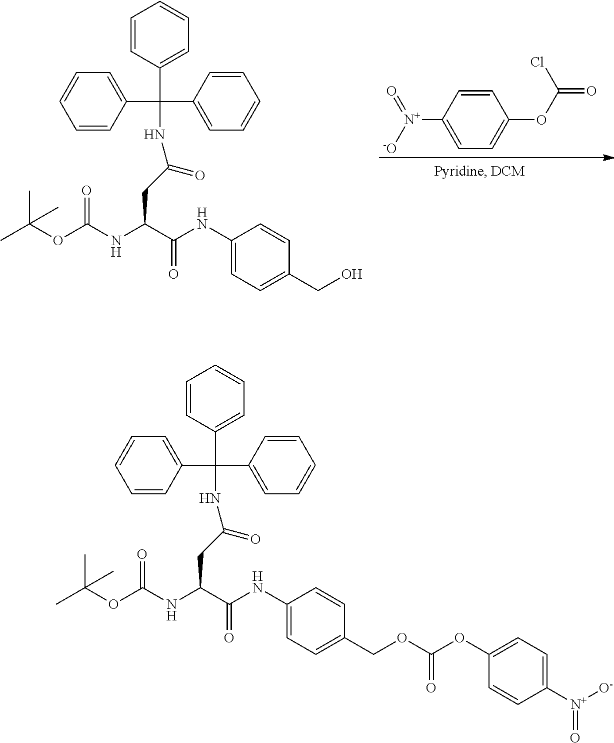

To a solution of tert-butyl N-[(1S)-1-{[4-(hydroxymethyl)phenyl]carbamoyl}-2-[(triphenylmethyl)carbamoyl]ethyl]carbamate (1000 mg, 1.73 mmol) in DCM (30 mL) were added 4-nitrophenyl carbonochloridate (695 mg, 3.45 mmol) and pyridine (0.279 mL, 3.45 mmol). The solution was stirred at room temperature for 3.5 hours. The resulting mixture was diluted with EtOAc and 10% aqueous citric acid. The organic layer was separated, washed with brine, dried over Na₂SO₄ and concentrated under reduced pressure. The residue was purified by silica gel column chromatography (EtOAc/heptane=1/4 to 1/1) to give {4-[(2S)-2-{[(tert-butoxy)carbonyl]amino}-3-[(triphenylmethyl)carbamoyl]propanamido]phenyl}methyl 4-nitrophenyl carbonate (1.26 g). LC-MS (ESI): 745.6 [M+H]⁺.

### Synthesis of {4-[(2S)-2-{[(tert-butoxy)carbonyl]amino}-3-[(triphenylmethyl)carbamoyl]propanamido]phenyl}methyl (2S)-2-(hydroxymethyl)pyrrolidine-1-carboxylate

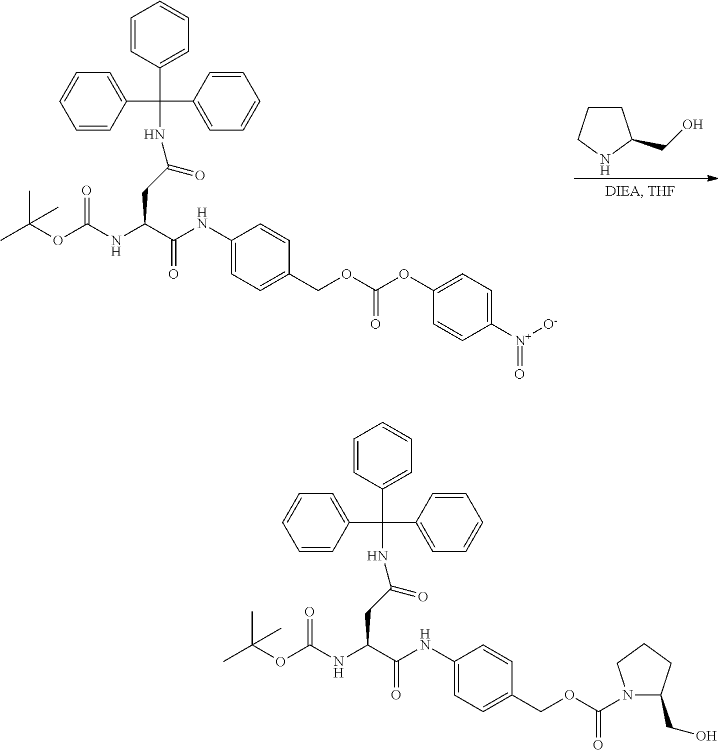 [SMILES: CC(C)(C)OC(=O)N[C@@H](CC(=O)NC(c1ccccc1)(c1ccccc1)c1ccccc1)C(=O)Nc1ccc(COC(=O)Oc2ccc([N+](=O)[O-])cc2)cc1 ; OC[C@@H]1CCCN1 ; CC(C)(C)OC(=O)N[C@@H](CC(=O)NC(c1ccccc1)(c1ccccc1)c1ccccc1)C(=O)Nc1ccc(COC(=O)N2CCC[C@H]2CO)cc1]

To a solution of {4-[(2S)-2-{[(tert-butoxy)carbonyl]amino}-3-[(triphenylmethyl)carbamoyl]propanamido]phenyl}methyl 4-nitrophenyl carbonate (400 mg, 0.537 mmol) in THF (10 mL) and DIEA (0.469 mL, 2.69 mmol) was added [(2S)-pyrrolidin-2-yl]methanol (65.2 mg, 0.644 mmol). The solution was stirred at room temperature for 2 h. The resulting mixture was concentrated under reduced pressure. The residue was purified by NH silica gel column chromatography (EtOAc/heptane=4/1 to 1/0) to give {4-[(2S)-2-{[(tert-butoxy)carbonyl]amino}-3-[(triphenylmethyl)carbamoyl]propanamido]phenyl}methyl (2S)-2-(hydroxymethyl)pyrrolidine-1-carboxylate (343.3 mg). LC-MS (ESI): 707.5 [M+H]⁺.

### Synthesis of {4-[(2S)-2-{[(tert-butoxy)carbonyl]amino}-3-[(triphenylmethyl)carbamoyl]propanamido]phenyl}methyl (2S)-2-({[(4-nitrophenoxy)carbonyl]oxy}methyl)pyrrolidine-1-carboxylate

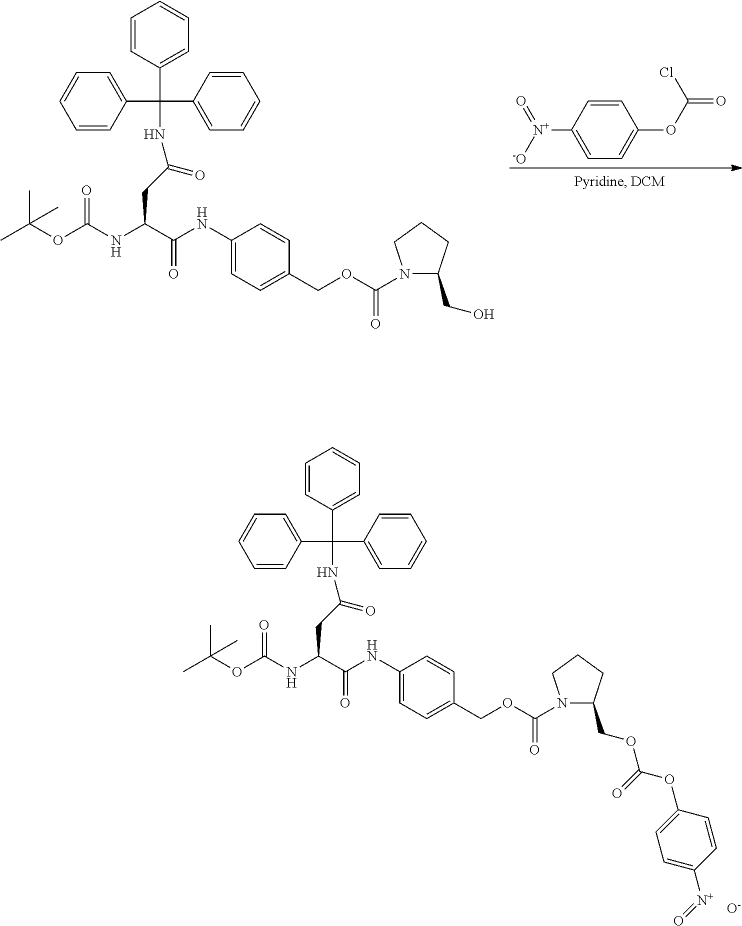

To a solution of {4-[(2S)-2-{[(tert-butoxy)carbonyl]amino}-3-[(triphenylmethyl)carbamoyl]propanamido]phenyl}methyl (2S)-2-(hydroxymethyl)pyrrolidine-1-carboxylate (343.3 mg, 0.486 mmol) in DCM (10 mL) were added 4-nitrophenyl carbonochloridate (196 mg, 0.971 mmol) and pyridine (0.079 mL, 0.971 mmol). The solution was stirred at room temperature for 2 h. The resulting mixture was diluted with EtOAc and 10% aqueous citric acid. The organic layer was separated, washed with brine, dried over Na₂SO₄, and concentrated under reduced pressure. The residue was purified by silica gel column chromatography (EtOAc/heptane=1/4 to 1/1) to give {4-[(2S)-2-{[(tert-butoxy)carbonyl]amino}-3-[(triphenylmethyl)carbamoyl]propanamido]phenyl}methyl (2S)-2-({[(4-nitrophenoxy)carbonyl]oxy}methyl)pyrrolidine-1-carboxylate (340 mg). LC-MS (ESI): 872.5 [M+H]⁺.

### Synthesis of [(2S)-1-[({4-[(2S)-2-{[(tert-butoxy)carbonyl]amino}-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate

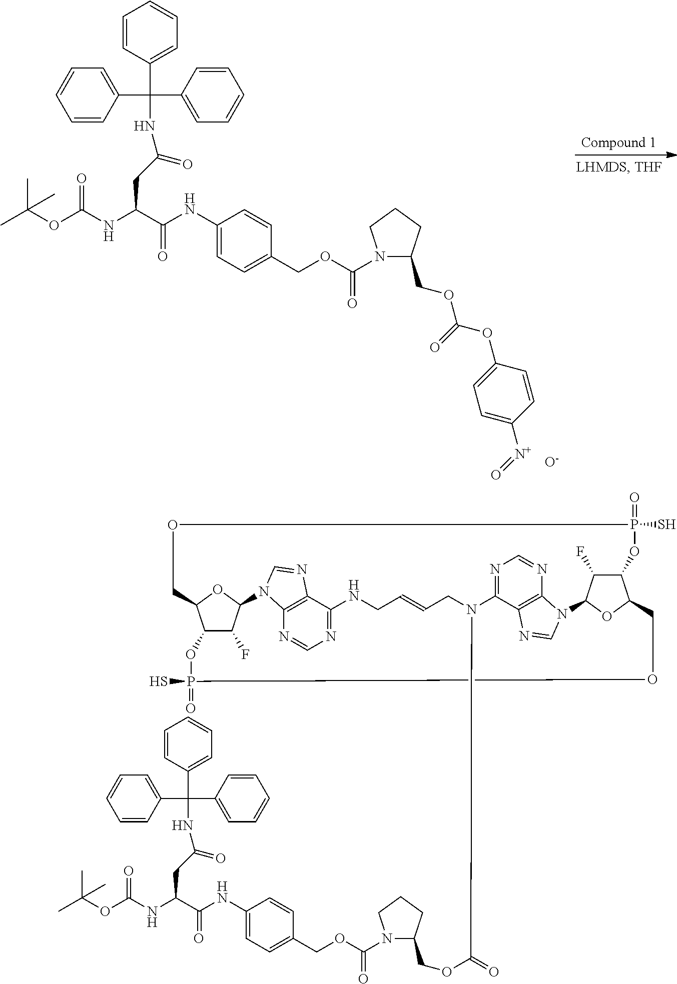

To a suspension of Compound 1 (50 mg, 0.059 mmol) in THF (5 mL) at room temperature was added LiHMDS (0.527 mmol). The resulting mixture was stirred for 30 min at room temperature and cooled to −78° C. To the above mixture was added {4-[(2S)-2-{[(tert-butoxy)carbonyl]amino}-3-[(triphenylmethyl)carbamoyl]propanamido]phenyl}methyl (2S)-2-({[(4-nitrophenoxy)carbonyl]oxy}methyl)pyrrolidine-1-carboxylate (45.9 mg, 0.053 mmol) in THF (0.5 mL) with THF rinse (0.5 mL) and stirred at −78° C. for 10 min. The resulting mixture was warmed to 0° C. and stirred for 1 h. The reaction was quenched with AcOH (226 mL, 3.95 mmol). The mixture was warmed to room temperature and stirred for 10 min. The resulting mixture was concentrated under reduced pressure. The residue was purified by ODS silica gel column chromatography (H₂O/MeCN including 0.1% formic acid=95/5 to 20/80) to give [(2S)-1-[({4-[(2S)-2-{[(tert-butoxy)carbonyl]amino}-3-[(triphenylmethyl)carbamoyl]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate (52.3 mg). LC-MS (ESI): 1480.6 [M+H]⁺.

### Synthesis of [(2S)-1-[({4-[(2S)-3-carbamoyl-2-[6-(2,5-dioxo-2,5-dihydro-1H-pyrrol-1-yl)hexanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate bis(triethylamine) salt (LP23.2NEt₃)

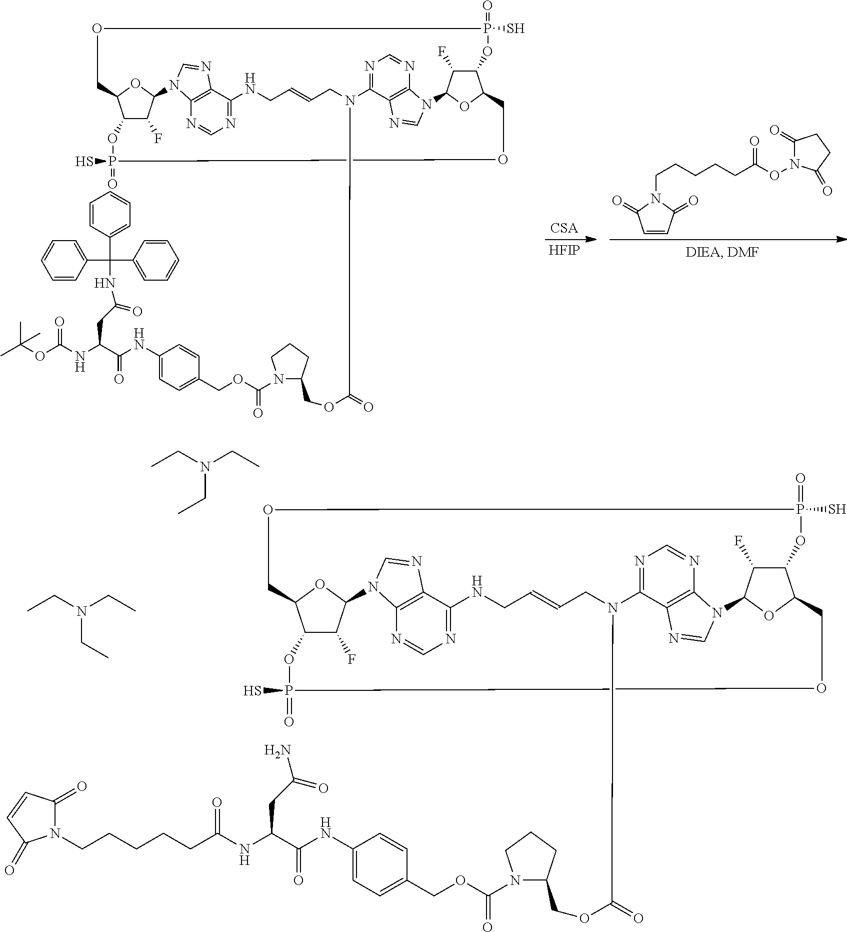

To a solution of [(2S)-1-[({4-[(2S)-2-{[(tert-butoxy)carbonyl]amino}-3-[(triphenyl methyl)carbamoyl]propanamido]phenyl})methoxy)carbonyl]pyrrolidin-2-yl]methyl (1R,3R,15E,28R,29R,30R,31R,345,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19, 21, 23,25-nonaene-13-carboxylate (45 mg, 0.03 mmol) in 1,1,1,3,3,3-hexafluoropropan-2-ol (4 mL) at 0° C. was added CSA (141 mg, 0.608 mmol). The mixture was stirred for 4 h at 0° C. The reaction mixture was quenched with MeOH. The resulting mixture was concentrated under reduced pressure. The residue was purified by ODS silica gel column chromatography (H₂O/MeCN including 0.1% formic acid=98/2 to 50/50) to give a crude compound (191.4 mg). A part of the obtained crude material (63.8 mg) in DMF (500 mL) and DIEA (52.9 mL, 0.303 mmol) was added 2,5-dioxopyrrolidin-1-yl 6-(2,5-dioxo-2,5-dihydro-1H-pyrrol-1-yl)hexanoate (2.80 mg, 9.09 mmol). The mixture was stirred for 1 h. To the mixture was added 2,5-dioxopyrrolidin-1-yl 6-(2,5-dioxo-2,5-dihydro-1H-pyrrol-1-yl)hexanoate (12 mg) and stirred for 30 min. To the mixture was added 2,5-dioxopyrrolidin-1-yl 6-(2,5-dioxo-2,5-dihydro-1H-pyrrol-1-yl)hexanoate (18 mg) and stirred for 30 min. The resulting mixture was concentrated under reduced pressure. The residue was purified by ODS silica gel column chromatography (H₂O/MeCN including 0.1% formic acid=98/2 to 50/50) to give crude material. The crude material was purified by normal Prep-TLC (CHCl₃/MeOH including 1% TEA=4/1) to give [(25)-1-[({4-[(2S)-3-carbamoyl-2-[6-(2,5-dioxo-2,5-dihydro-1H-pyrrol-1-yl)hexanamido]propanamido]phenyl}methoxy)carbonyl]pyrrolidin-2-yl]methyl (1R,3R,15E,28R,29R,30R,31R,34S,36R,39R,41R)-29,41-difluoro-34,39-dioxo-34,39-disulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaene-13-carboxylate bis(triethylamine) salt (LP23.2NEt₃) (3.35 mg).

LC-MS (ESI): 1330.8 [M+H]⁺.

### 31. Syntheses of LP32 and LP31

The syntheses LP32 and LP31 are shown below:

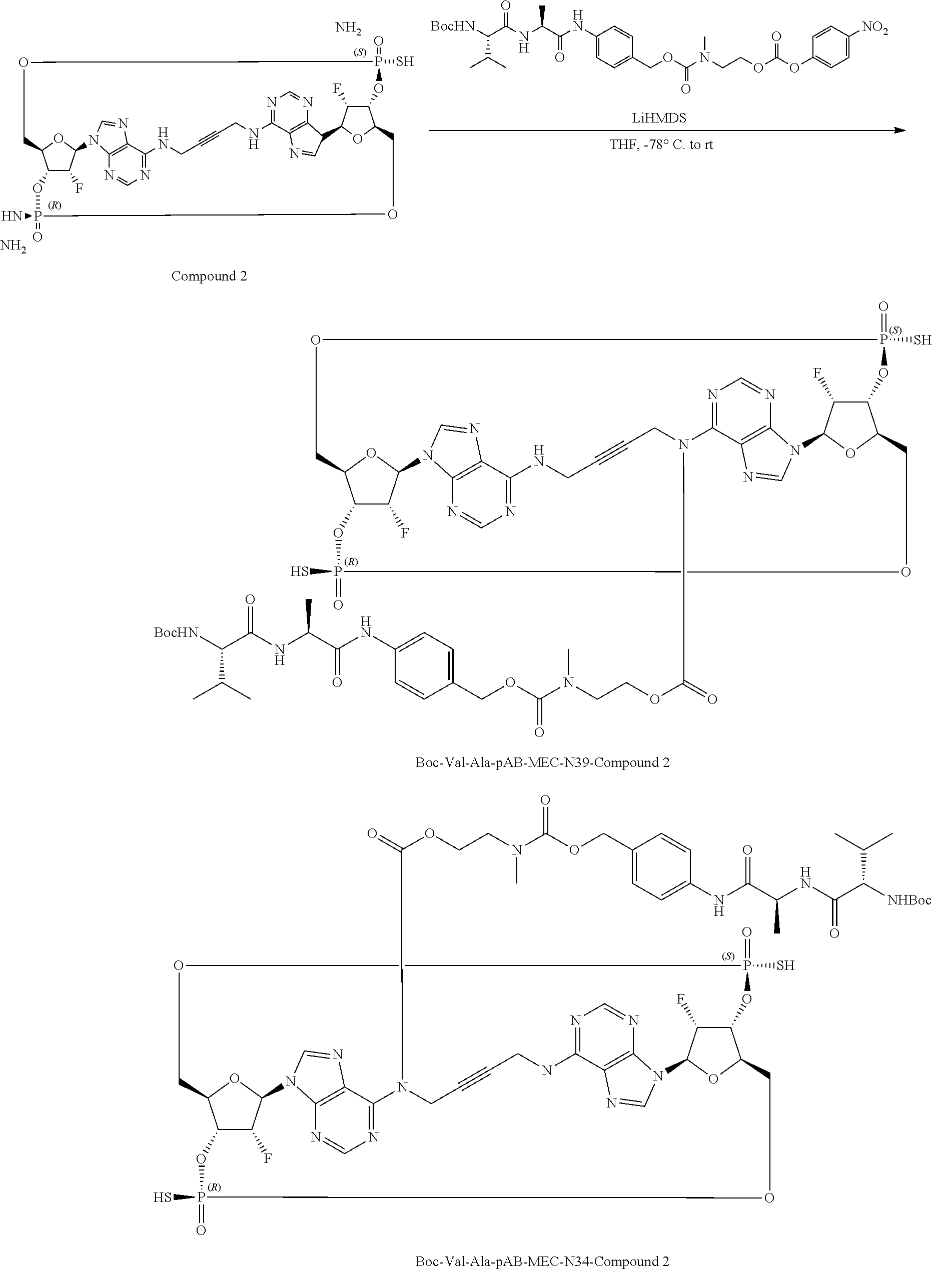

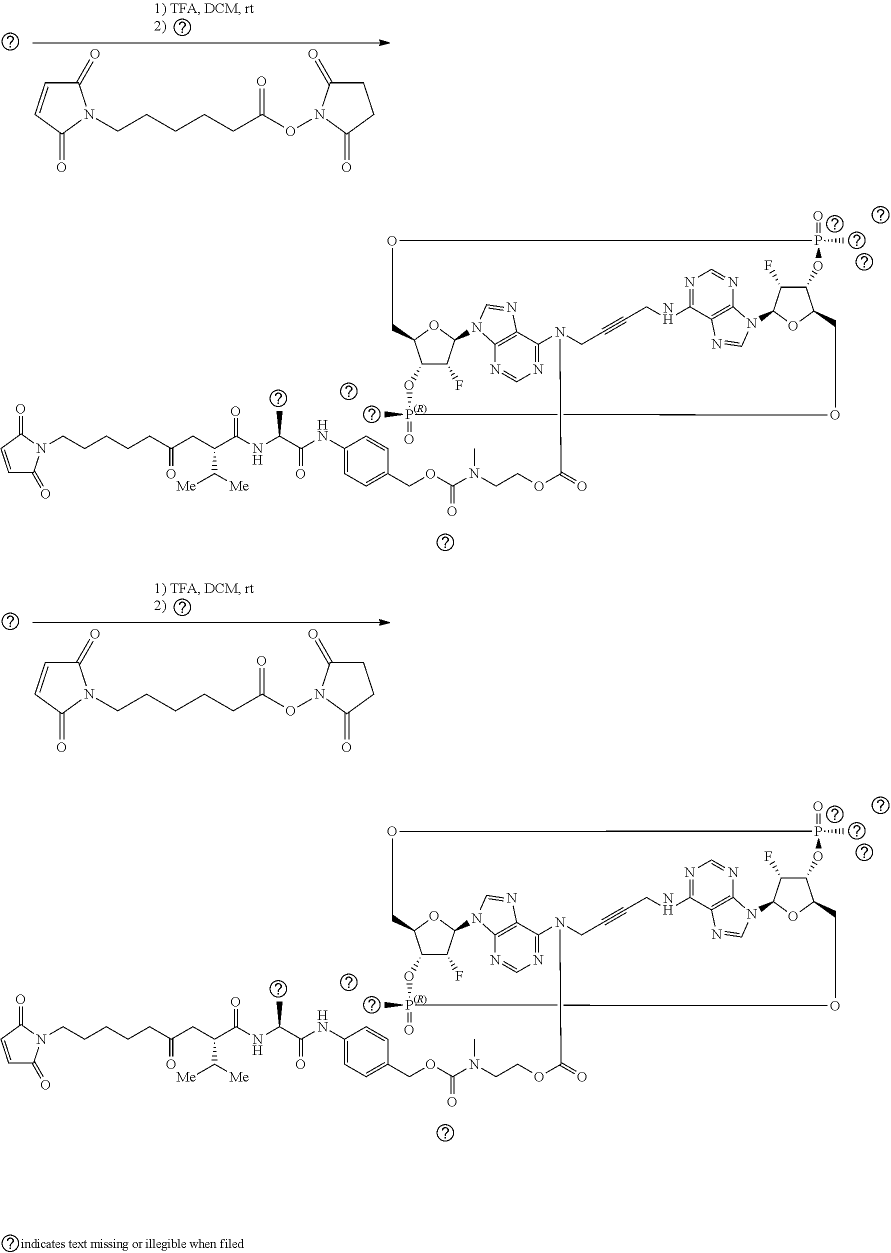

### Synthesis of Boc-Val-Ala-pAB-MEC-(N39)-Compound 2 and Boc-Val-Ala-pAB-MEC-(N34)-Compound 2

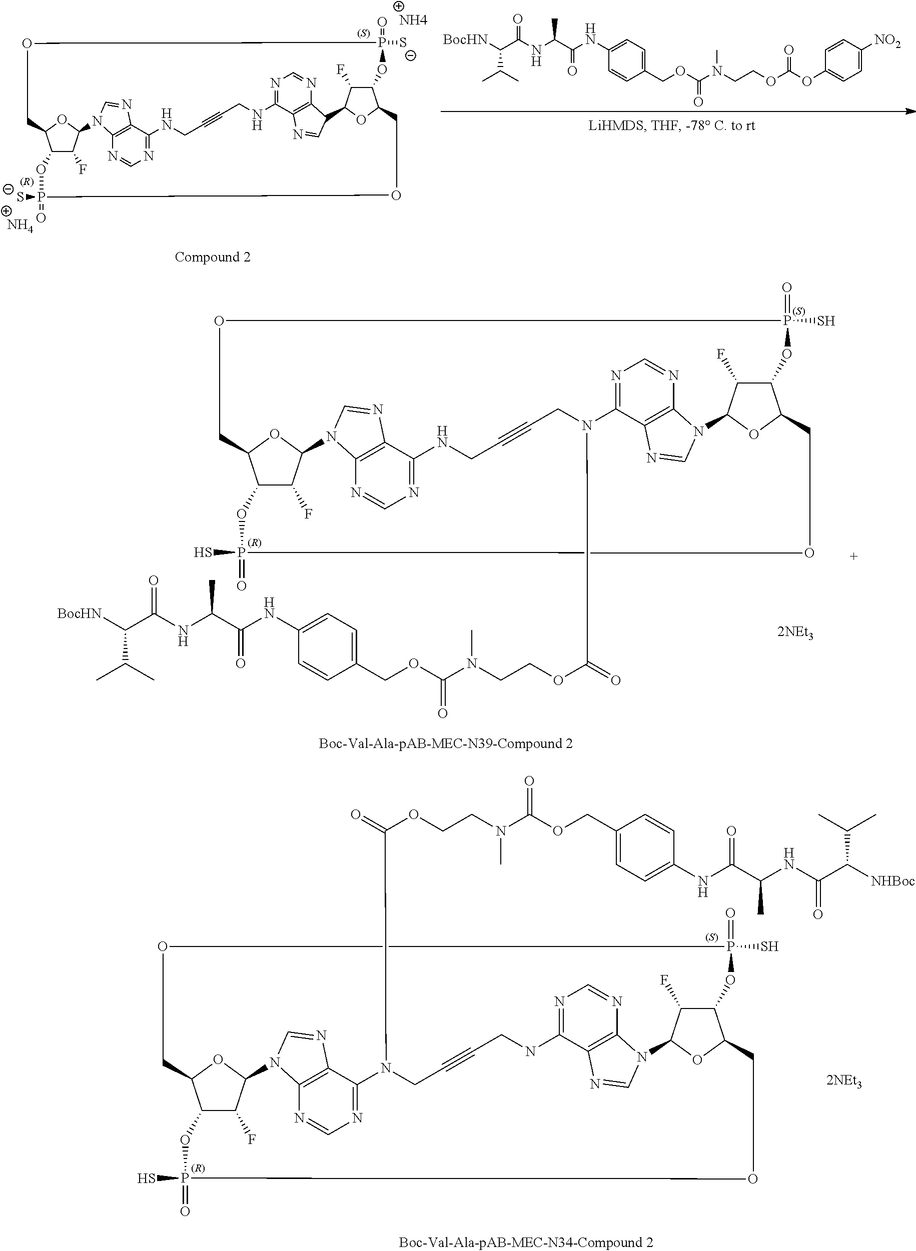

To diammonium salt of Compound 2 (50 mg, 0.064 mmol) was added THF (3 mL) and the resulting slurry was concentrated in vacuo. This azeotroping drying process was repeated two more times. The resulting material was dissolved in THF (5 mL) and treated with a solution of LiHMDS (1.5 M, 0.385 mL, 0.578 mmol) in THF at rt. After 10 min at rt, the reaction mixture was cooled down to−78° C. A solution of 4-((S)-2-((S)-2-((tert-butoxycarbonyl)amino)-3-methylbutanamido)propanamido)benzyl methyl(2-(((4-nitrophenoxy)carbonyl)oxy)ethyl)carbamate (42.4 mg, 0.064 mmol) in THF (0.84 mL) was then added. The resulting mixture was slowly warmed to 0° C. over 1.5 h. After 1.5 h between 0-10° C., the reaction was quenched with AcOH (0.1 mL, 1.7 mmol). The resulting mixture was concentrated under reduced pressure. The residue was purified by Prep-HPLC under the following conditions; Column: XBridge Prep OBD C18 Column, 19*100 mm, 5 μm; Mobile Phase A: Water (50 mM TEA+50 mM hexafluoroisopropanol), Mobile Phase B: MeCN (50 mM TEA+50 mM hexafluoroisopropanol); Flow rate: 40 mL/min; Temperature:rt; Gradient: 1% B (t=0-2.0 min), 15% B (t=2.1 min), 35% B (t=16.3 min), 95% B (t=16.4 min), 1% B(t=17.2-17.9 min); Wavelength: 260 nm; Runtime=18 min). This resulted in 18.9 mg of Boc-Val-Ala-pAB-MEC-(N39)-Compound 2 and 5.2 mg of Boc-Val-Ala-pAB-MEC-(N34)-Compound 2.

Boc-Val-Ala-pAB-MEC-(N39)-Compound 2: LC-MS (ESI): 1265.32 [M+H]⁺. ¹H NMR (400 MHz, Methanol-d₄) δ 8.88 (br s, 1H), 8.78-8.64 (m, 1H), 8.59 (br s, 1H), 8.16 (s, 1H), 7.61-7.50 (m, 2H), 7.28 (br d, J=7.5 Hz, 1H), 7.19 (br d, J=7.3 Hz, 1H), 6.45-6.24 (m, 2H), 6.04 (d, J=51.4 Hz, 1H), 5.51 (d, J=51.3 Hz, 1H), 5.17-4.92 (m, 2H), 4.95-4.54 (m, 5H), 4.49 (br d, J=10.4 Hz, 2H), 4.44-4.30 (m, 3H), 4.29-4.12 (m, 1H), 4.02 (br dd, J=3.6, 11.4 Hz, 1H), 3.98-3.87 (m, 2H), 3.86-3.70 (m, 1H), 3.65-3.40 (m, 2H), 3.35 (s, 1H), 3.25-3.11 (m, 12H), 2.83-2.70 (m, 3H), 2.07 (br d, J=4.6 Hz, 1H), 1.44 (br s, 12H), 1.28 (br t, J=7.3 Hz, 18H), 0.98 (br d, J=6.5 Hz, 3H), 0.93 (br d, J=6.4 Hz, 3H). Analytical HPLC RT=9.70 min [Column: Xbridge Premier BEH C18 Column, 2.5 μm (2.1 mm ID×150 mm); Mobile Phase A: Water (50 mM TEA+50 mM hexafluoroisopropanol), Mobile Phase B: MeCN (50 mM TEA+50 mM hexafluoroisopropanol); Flow rate: 0.5 mL/min; Temperature:60; Gradient: 1% B (t=0-1.0 min), 15% B (t=1.1 min), 35% B (t=20.9 min), 95% B (t=21.0 min), 1% B(t=22.1-24.0 min); Wavelength: 260 nm; Runtime=25 min].

Boc-Val-Ala-pAB-MEC-(N34)-Compound 2: LC-MS (ESI): 1265.32 [M+H]⁺. ¹H NMR (400 MHz, Methanol-d₄) δ 9.30 (br s, 0.5H), 9.09 (br s, 0.5H), 8.83-8.63 (m, 1H), 8.47 (br s, 1H), 8.13 (br s, 0.5H), 7.97 (br s, 0.5H), 7.61-7.51 (m, 2H), 7.28 (br d, J=7.0 Hz, 1H), 7.20 (br d, J=7.4 Hz, 1H), 6.54-6.15 (m, 2H), 5.94-5.28 (m, 2H), 5.22-5.06 (m, 1H), 4.97 (br s, 1H), 4.82-4.57 (m, 5H), 4.49 (br d, J=7.0 Hz, 2H), 4.41 (br s, 2H), 4.35-4.11 (m, 2H), 4.04 (br d, J=9.6 Hz, 1H), 3.99-3.76 (m, 3H), 3.60-3.43 (m, 1H), 3.37-3.34 (m, 2H), 3.26-3.09 (m, 12H), 2.76 (br s, 3H), 2.17-2.00 (m, 1H), 1.44 (s, 12H), 1.30 (t, J=7.3 Hz, 18H), 0.98 (br d, J=6.5 Hz, 3H), 0.94 (br d, J=6.6 Hz, 3H). Analytical HPLC RT=8.98 [Column: Xbridge Premier BEH C18 Column, 2.5 μm (2.1 mm ID×150 mm); Mobile Phase A: Water (50 mM TEA+50 mM hexafluoroisopropanol), Mobile Phase B: MeCN (50 mM TEA+50 mM hexafluoroisopropanol); Flow rate: 0.5 mL/min; Temperature:60; Gradient: 1% B (t=0-1.0 min), 15% B (t=1.1 min), 35% B (t=20.9 min), 95% B (t=21.0 min), 1% B(t=22.1-24.0 min); Wavelength: 260 nm; Runtime=25 min].

### Synthesis of Val-Ala-pAB-MEC-(N39)-Compound 2

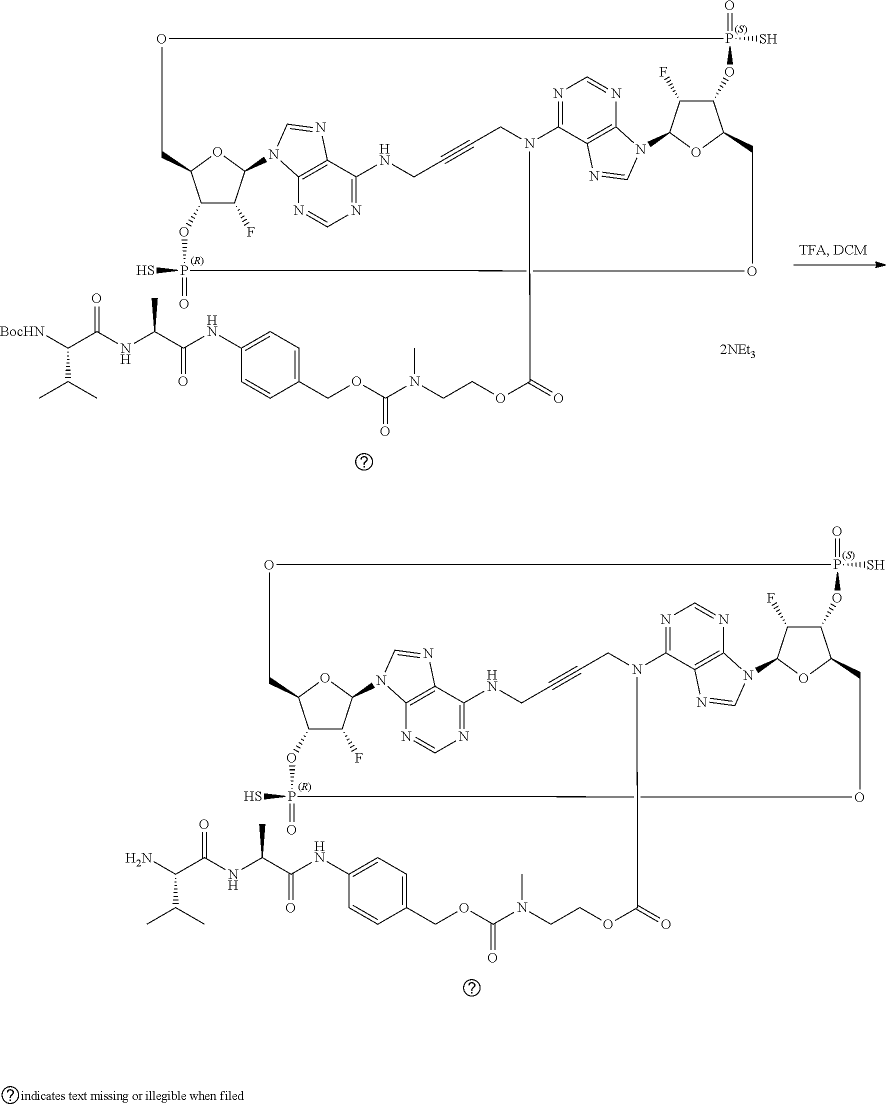

To a solution of Boc-Val-Ala-pAB-MEC-(N39)-Compound 2 (18.9 mg, 0.013 mmol) in DCM (2 mL) at rt was added TFA (0.75 mL). The reaction mixture was stirred for 0.5 h at rt and then treated with MTBE (5 mL). The resulting slurry mixture was subjected to centrifuge. Decantation followed by drying the resulting solid in vacuo overnight provided 16.5 mg of the target product as an off-white solid. LC-MS (ESI): 1165.05 [M+H]⁺.

### Synthesis of Val-Ala-pAB-MEC-(N34)-Compound 2

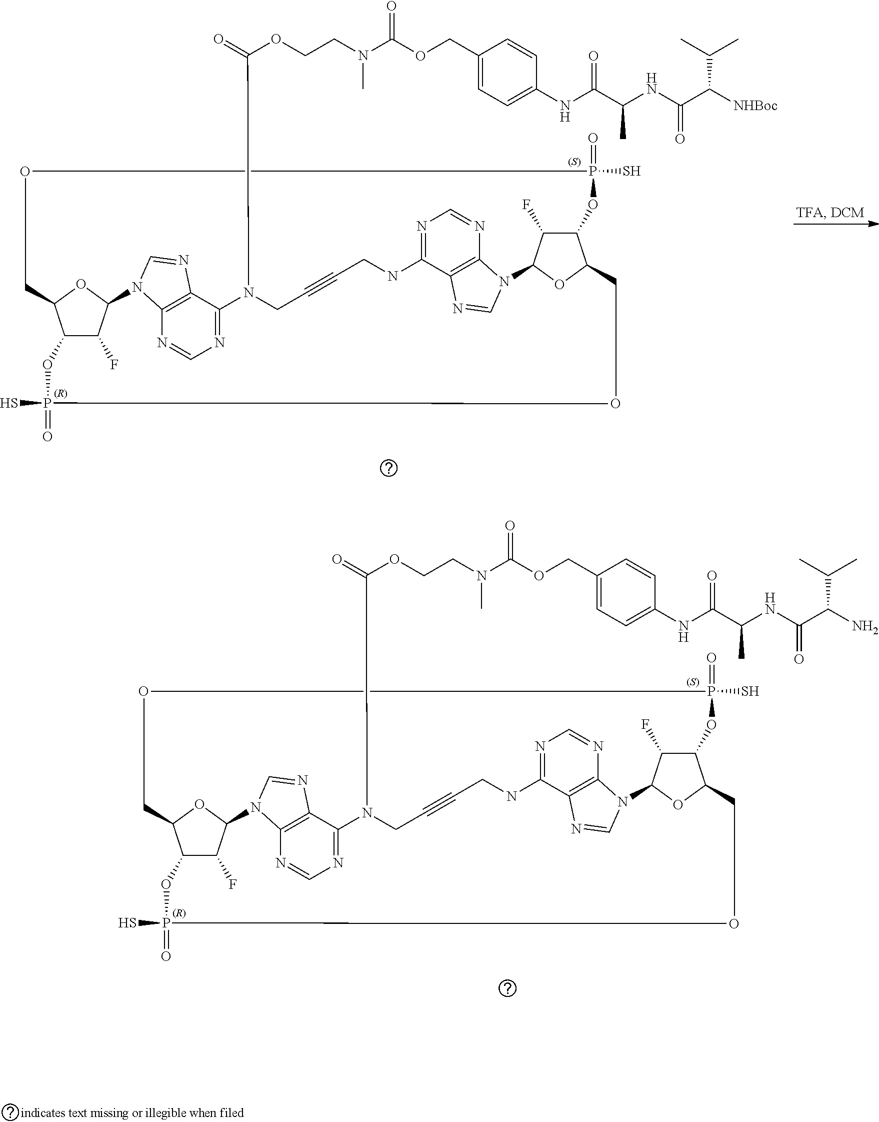

To a solution of Boc-Val-Ala-pAB-MEC-(N34)-Compound 2 (5.2 mg, 0.0052 mmol) in DCM (0.8 mL) at rt was added TFA (0.3 mL). The reaction mixture was stirred for 0.5 h at rt and then treated with MTBE (2.5 mL). The resulting slurry mixture was subjected to centrifuge. Decantation followed by drying the resulting solid in vacuo overnight provided 4.0 mg of the target product as an off-white solid. LC-MS (ESI): 1165.10 [M+H]⁺.

### Synthesis of LP32

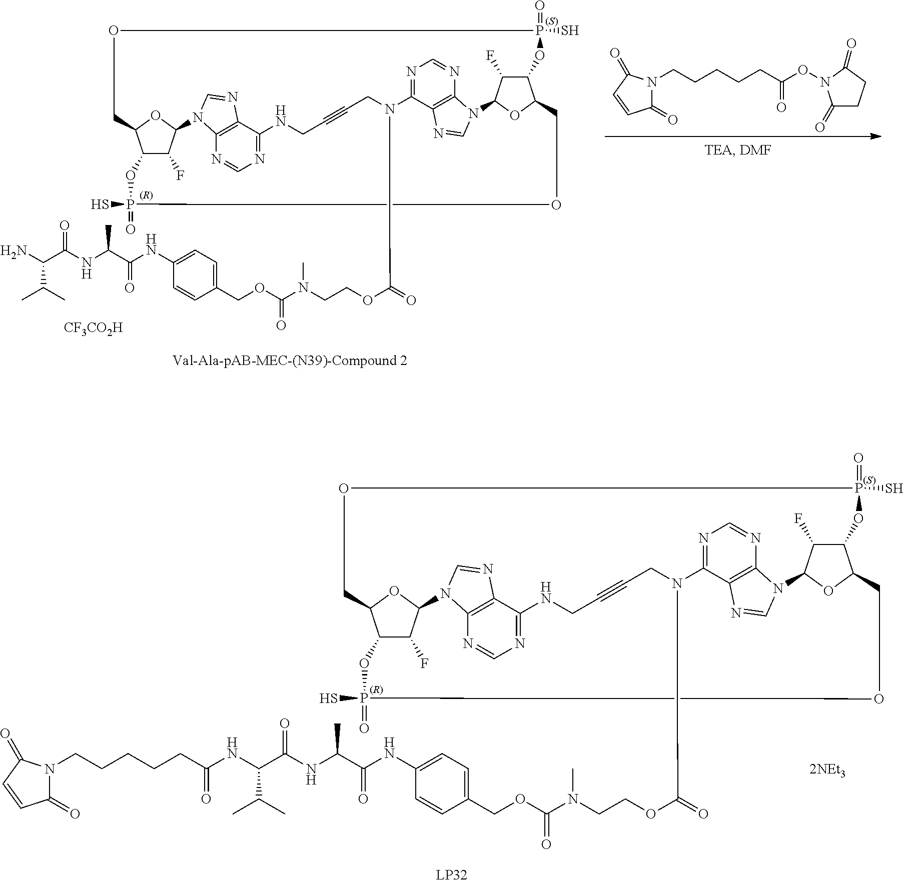

To a solution of Val-Ala-pAB-MEC-(N39)-Compound 2 (5.3 mg, 0.0041 mmol) in DMF (0.5 mL) at rt were added TEA (0.010 mL, 0.072 mmol) and 2,5-dioxopyrrolidin-1-yl 6-(2,5-dioxopyrrol-1-yl)hexanoate (2.0 mg, 0.0065 mmol). The resulting mixture was stirred at rt for 10 min and treated with additional 2,5-dioxopyrrolidin-1-yl 6-(2,5-dioxopyrrol-1-yl)hexanoate (2.0 mg, 0.0065 mmol). After being stirred for 3 h at rt, the reaction mixture was kept at −20° C. over weekend, warmed to rt, and purified by silica gel column chromatography (15 to 30% MeOH in EtOAc buffered with 1% triethylamine). This resulted in 3.2 mg of target product LP32-2NEt₃ as a white solid.

LC-MS (ESI): 1358.00 [M+H]⁺.

¹H NMR (400 MHz, Methanol-d₄) δ 8.89 (br s, 1H), 8.77-8.65 (m, 1H), 8.63-8.53 (m, 1H), 8.16 (br s, 1H), 7.56 (br dd, J=7.9, 14.3 Hz, 2H), 7.37-7.25 (m, 1H), 7.19 (br d, J=7.3 Hz, 1H), 6.77 (br s, 2H), 6.42-6.22 (m, 2H), 6.04 (d, J=49.8 Hz, 1H), 5.51 (d, J=49.5 Hz, 1H), 5.15-4.92 (m, 3H), 4.82-4.53 (m, 4H), 4.52-4.25 (m, 5H), 4.24-4.12 (m, 2H), 4.02 (br dd, J=3.8, 11.4 Hz, 1H), 3.93 (br d, J=8.4 Hz, 1H), 3.88-3.70 (m, 1H), 3.67-3.52 (m, 1H), 3.50-3.35 (m, 4H), 3.24-3.13 (m, 12H), 2.78 (br s, 3H), 2.28 (br s, 2H), 2.15-2.05 (m, 1H), 1.67-1.49 (m, 4H), 1.44 (br d, J=6.9 Hz, 3H), 1.29 (br t, J=7.3 Hz, 18H), 1.18-1.05 (m, 2H), 0.99-0.88 (m, 6H).

### Synthesis of LP31

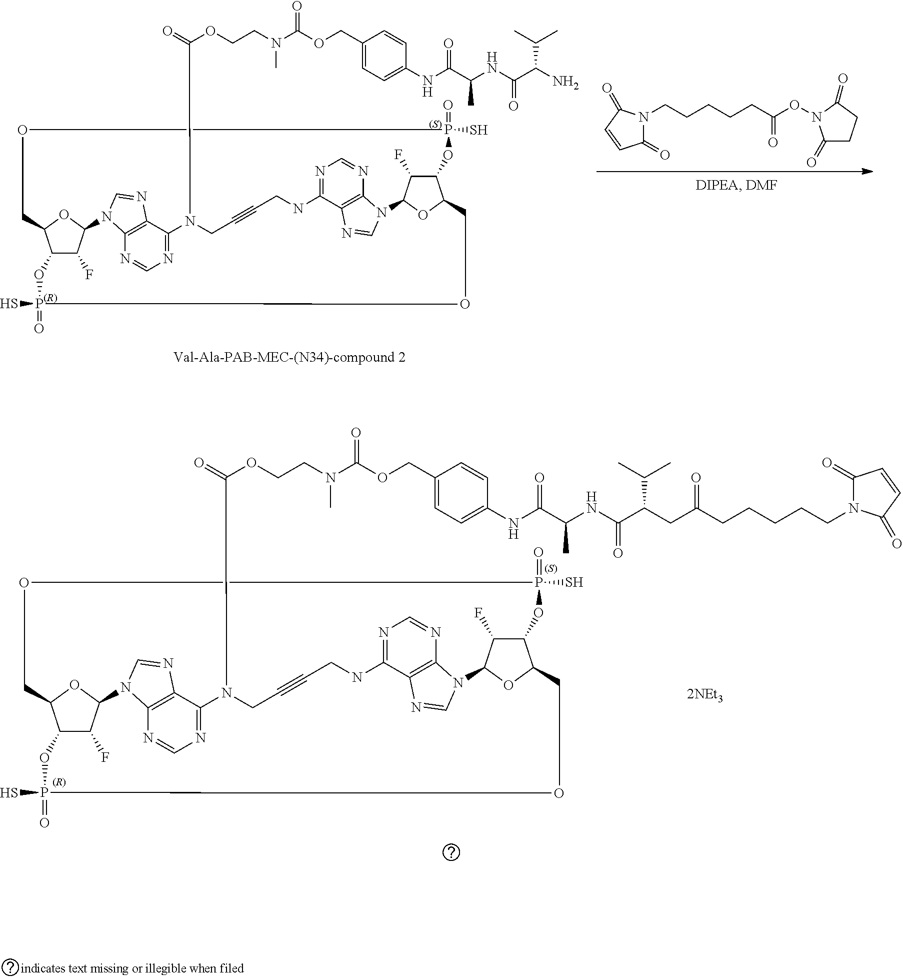

To a solution of Val-Ala-pAB-MEC-(N34)-Compound 2 (4.0 mg, 0.0031 mmol) in DMF (0.4 mL) at rt were added TEA (0.010 mL, 0.072 mmol) and 2,5-dioxopyrrolidin-1-yl 6-(2,5-dioxopyrrol-1-yl)hexanoate (4.0 mg, 0.013 mmol). After being stirred for 8 h at rt, the reaction mixture was purified by silica gel column chromatography (15 to 30% MeOH in EtOAc buffered with 1% triethylamine). This resulted in 3.3 mg of target product LP31-2NEt₃ as a white solid.

LC-MS (ESI): 1359.96 [M+H]⁺.

¹H NMR (400 MHz, Methanol-d₄) δ 9.40-9.25 (m, 0.5H), 9.15-9.03 (m, 0.5H), 8.85-8.62 (m, 1H), 8.55-8.39 (m, 1H), 8.23-7.91 (m, 1H), 7.62-7.49 (m, 2H), 7.33-7.25 (m, 1H), 7.22-7.12 (m, 1H), 6.78 (s, 2H), 6.56-6.17 (m, 2H), 5.93-5.31 (m, 2H), 5.23-5.06 (m, 1H), 5.01-4.93 (m, 1H), 4.74-4.54 (m, 5H), 4.53-4.24 (m, 5H), 4.18 (br d, J=7.0 Hz, 2H), 4.09-4.00 (m, 1H), 3.98-3.89 (m, 1H), 3.86-3.70 (m, 1H), 3.58-3.38 (m, 5H), 3.28-3.12 (m, 12H), 2.76 (br s, 3H), 2.28 (br t, J=6.7 Hz, 2H), 2.17-2.01 (m, 1H), 1.69-1.50 (m, 4H), 1.45 (br d, J=6.3 Hz, 3H), 1.31 (t, J=7.2 Hz, 18H), 1.21-1.07 (m, 2H), 1.04-0.90 (m, 6H).

### 32. Synthesis of LP33 and Alternative Synthesis of LP3

The synthesis of LP3 is shown below:

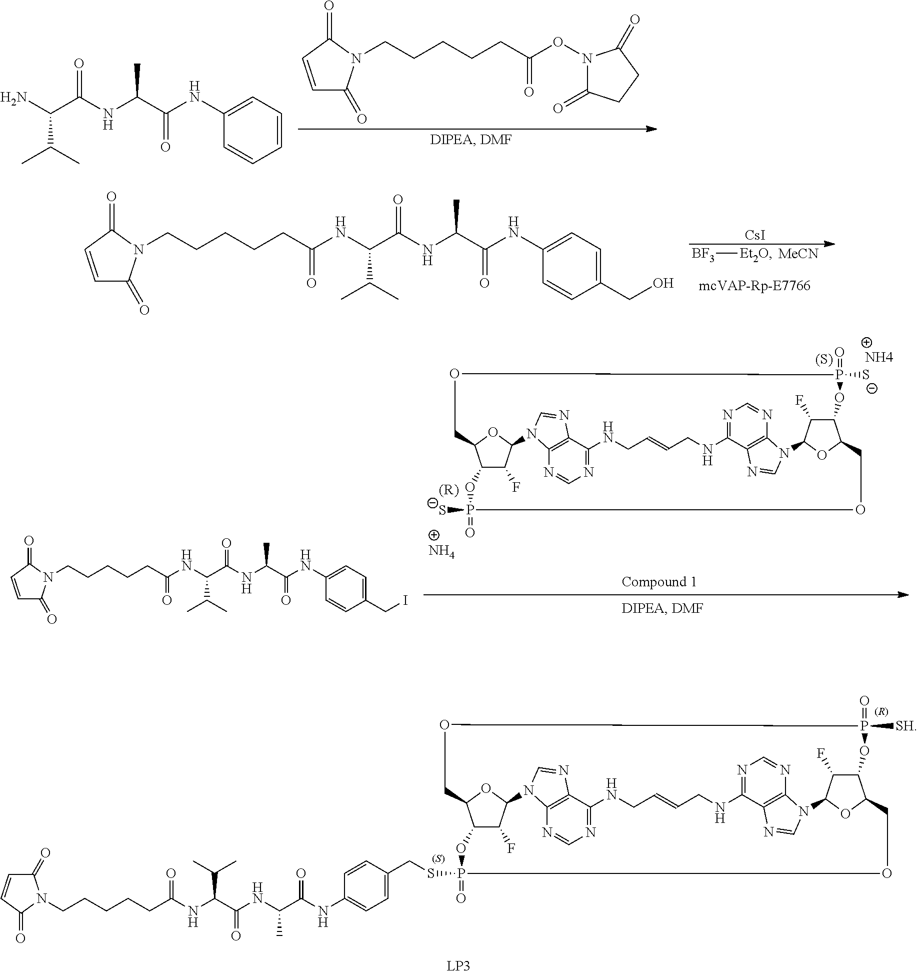

### Synthesis of 6-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]hexanamide

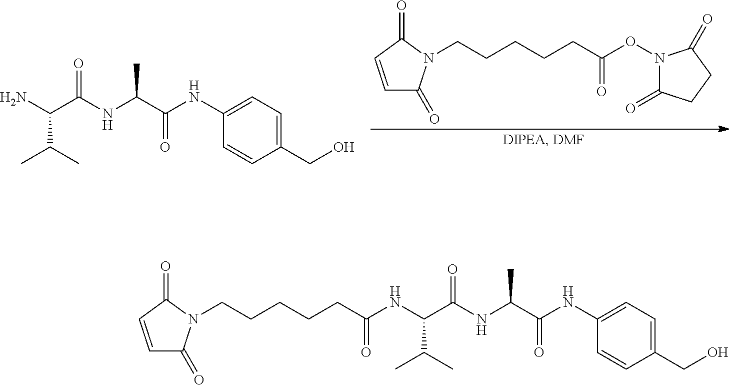 [SMILES: CC(C)[C@H](N)C(=O)N[C@@H](C)C(=O)Nc1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1]

DIPEA (1.32 g, 10.2 mmol) and 2,5-dioxopyrrolidin-1-yl 6-(2,5-dioxo-2,5-dihydro-1H-pyrrol-1-yl)hexanoate (1.58 g, 5.11 mmol) were added to a solution of (2S)-2-amino-N-[(1S)-1-{[4-(hydroxymethyl)phenyl]carbamoyl}ethyl]-3-methylbutanamide (1.5 g, 5.1 mmol) in DMF (40 mL). The resulting mixture was stirred at rt for 2 h. The resulting mixture was diluted with EtOAc, washed with water, and concentrated. The residue was purified by silica gel column chromatography, eluted with CH₂Cl₂/MeOH (10:1). This resulted in 2.0 g of 6-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]hexanamide as an off-white solid. LC-MS (ESI): 487 [M+H]⁺.

### Synthesis of 6-(2,5-dioxo-2,5-dihydro-1H-pyrrol-1-yl)-N-[(1S)-1-{[(1S)-1-{[4-(iodomethyl)phenyl]carbamoyl}ethyl]carbamoyl}-2-methylpropyl]hexanamide

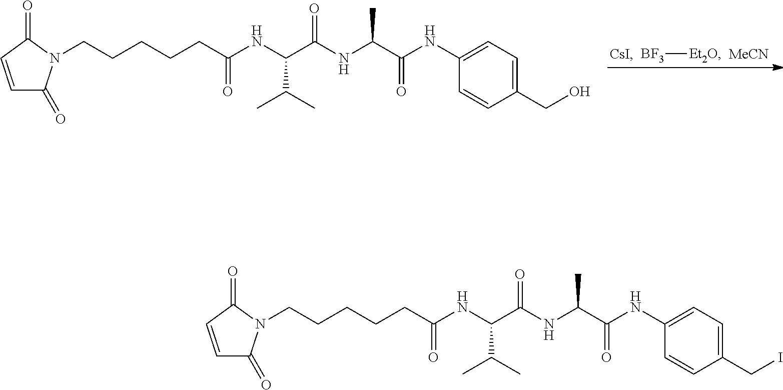

Cesium iodide (801 mg, 3.08 mmol) and BF3·Et₂O (438 mg, 3.08 mmol) were added to a solution of 6-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]hexanamide (1.0 g, 2.06 mmol) in acetonitrile (10 mL). The resulting mixture was stirred at rt for 2 h. The resulting mixture was concentrated under reduced pressure. The residue was purified by silica gel column chromatography, eluted with CH₂Cl₂/ⁱPrOH (10:1). This resulted in 700 mg of 6-(2,5-dioxo-2,5-dihydro-1H-pyrrol-1-yl)-N-[(1S)-1-{[(1S)-1-{[4-(iodomethyl)phenyl]carbamoyl}ethyl]carbamoyl}-2-methylpropyl]hexanamide as a white solid. LC-MS (ESI): 597 [M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxopyrrol-1-yl)hexanamide (LP3) and N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP33)

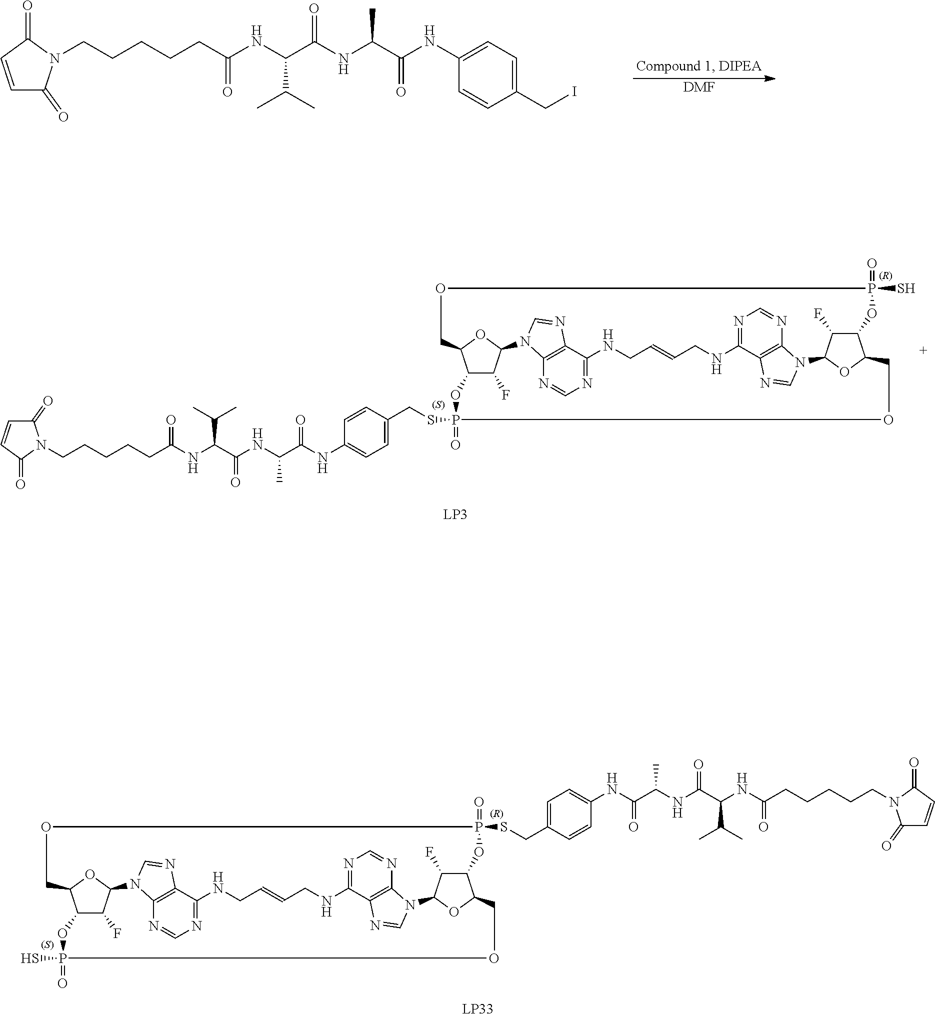

6-(2,5-Dioxo-2,5-dihydro-1H-pyrrol-1-yl)-N-[(1S)-1-{[(1S)-1-{[4-(iodomethyl)phenyl]carbamoyl}ethyl]carbamoyl}-2-methylpropyl]hexanamide (480 mg, 0.805 mmol) and DIPEA (208 mg, 1.61 mmol) were added to a solution of Compound 1 (600 mg, 0.805 mmol) in DMF (5 mL). The resulting mixture was stirred at rt for 2 hours. The crude solution was purified by Prep-HPLC with the following conditions (Column: XBridge Prep OBD C18 Column, 19*250 mm, 5 μm; Mobile Phase A: Water (0.05% FA), Mobile Phase B: MeCN; Flow rate: 25 mL/min; Gradient: 32% B to 32% B in 8 min, 32% B; Wavelength: 254 nm; RT=13 min). This resulted in 108 mg of target product LP3 and 78.9 mg of LP33.

LP3: LC-MS (ESI): 1215.3 [M+H]⁺. ¹H NMR (400 MHz, Methanol-d₄) δ (ppm)=8.77-8.46 (m, 1H), 8.30-7.90 (m, 6H), 7.40 (br s, 2H), 7.22-6.85 (m, 2H), 6.77 (s, 2H), 6.45 (br d, J=14.5 Hz, 1H), 6.25 (br d, J=19.9 Hz, 1H), 5.90-5.10 (m, 6H), 4.75-3.50 (m, 14H), 3.46 (t, J=7.0 Hz, 2H), 2.28 (t, J=7.4 Hz, 2H), 2.16-2.04 (m, 1H), 1.70-1.50 (m, 4H), 1.47-1.37 (m, 3H), 1.35-1.24 (m, 2H), 0.97 (t, J=7.0 Hz, 6H). Analytical HPLC RT=6.74 min (Instrument: Shimadzu LC20AD; Column: HALO C18 (4.6 mm ID×100 mm); Mobile Phase A: Water (0.05% TFA), Mobile Phase B: MeCN (0.05% TFA); Flow rate: 1.5 mL/min; Temperature: 40° C.; Gradient: 10% B (t=0.01 min), 70% B (t=10 min), 95% B (t=12 to 14 min); Wavelength: 254 nm)

LP33: LC-MS (ESI): 1215.3 [M+H]⁺. ¹H NMR (400 MHz, Methanol-d4) δ 8.75-8.43 (m, 1H), 8.37-7.79 (m, 3H), 7.70-7.37 (m, 4H), 6.89-6.78 (m, 2H), 6.57-6.22 (m, 2H), 5.98-5.51 (m, 2H), 4.62-4.35 (m, 4H), 4.36-4.21 (m, 3H), 4.18-3.96 (m, 4H), 3.93-3.71 (m, 1H), 3.54-3.42 (m, 2H), 2.34-2.24 (m, 2H), 2.16-2.01 (m, 1H), 1.70-1.51 (m, 4H), 1.44-1.36 (m, 3H), 1.36-1.26 (m, 3H), 1.06-0.89 (m, 6H). Analytical HPLC RT=6.64 min (Instrument: Shimadzu LC20AD; Column: HALO C18 (4.6 mm ID×100 mm); Mobile Phase A: Water (0.05% TFA), Mobile Phase B: MeCN (0.05% TFA); Flow rate: 1.5 mL/min; Temperature: 40° C.; Gradient: 10% B (t=0.01 min), 70% B (t=10 min), 95% B (t=12 to 14 min); Wavelength: 254 nm)

### 33. Synthesis of LP34

The synthesis of LP34 is shown below:

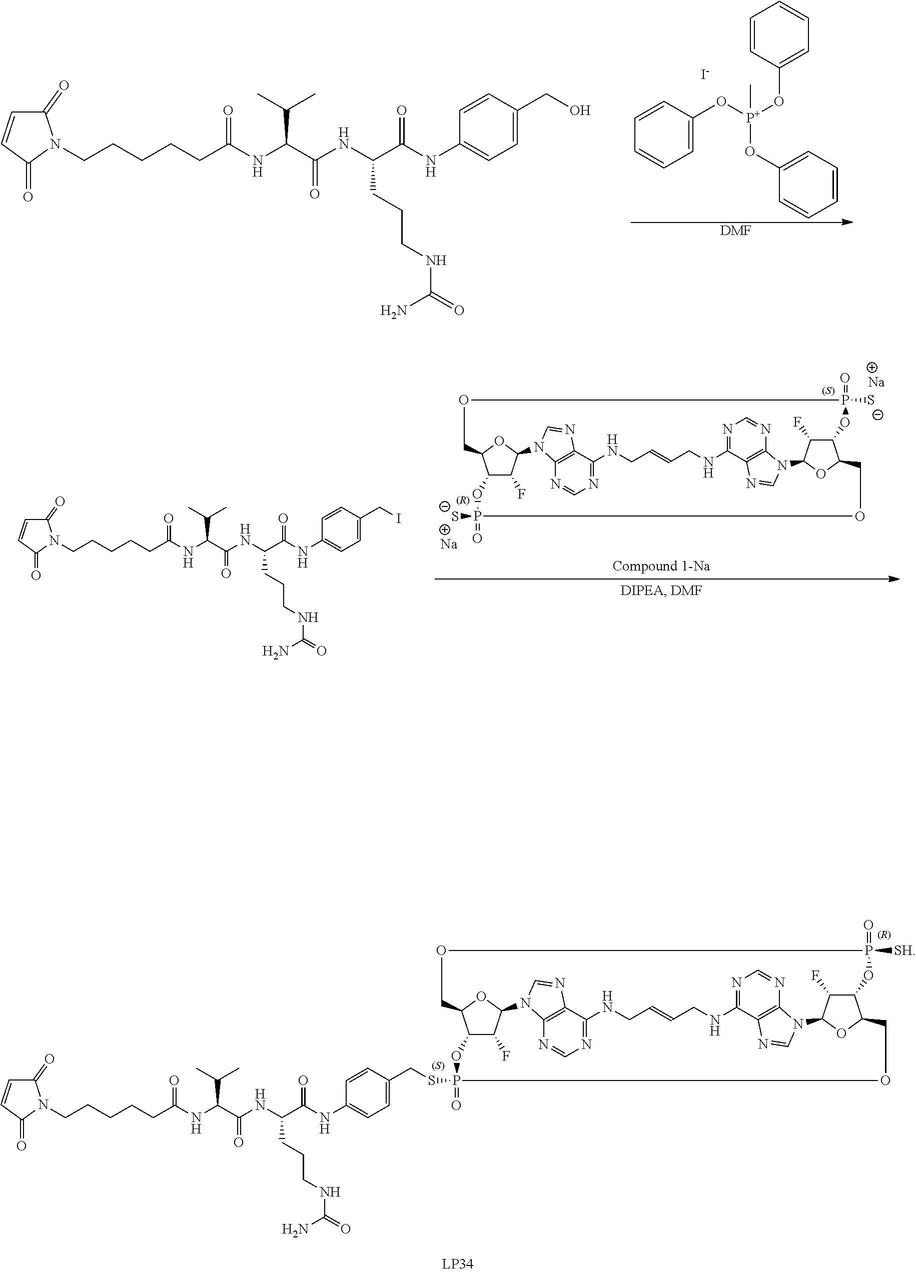

### Synthesis of 6-(2,5-dioxo-2,5-dihydro-1H-pyrrol-1-yl)-N-((S)-1-(((S)-1-((4-(iodomethyl)phenyl)amino)-1-oxo-5-ureidopentan-2-yl)amino)-3-methyl-1-oxobutan-2-yl)hexanamide

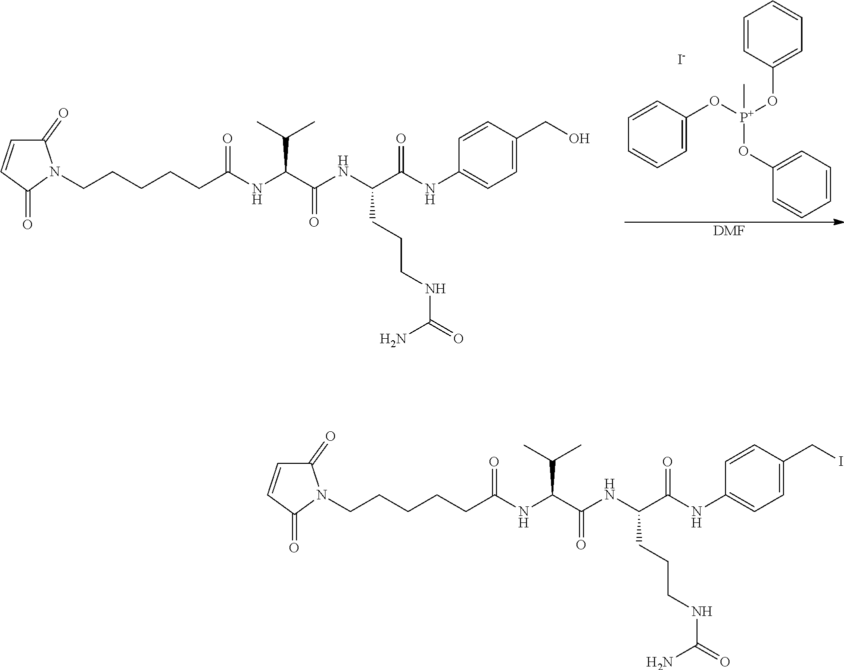

To a solution of 6-(2,5-dioxo-2,5-dihydro-1H-pyrrol-1-yl)-N-((S)-1-(((S)-1-((4-(hydroxymethyl)phenyl)amino)-1-oxo-5-ureidopentan-2-yl)amino)-3-methyl-1-oxobutan-2-yl)hexanamide (55 mg, 0.096 mmol) in DMF (0.5 mL) at rt was added methyltriphenoxyphosphonium iodide (90 mg, 0.199 mmol). The resulting mixture was stirred in dark at rt overnight. Additional methyltriphenoxyphosphonium iodide (30 mg) was added. After 4 h, the reaction mixture was diluted with 20 mL EtOAc and washed with a sat'd solution of Na₂S₂O₃ (10 mL). The aqueous layer was extracted with EtOAc (10 mL). The combined organic layers were washed with 30% NaCl aqueous solution (5 mL) and dried over Na₂SO₄, filtered and concentrated in vacuo. The resulting solid was further dried in vacuo and used in next step without further purification. LC-MS (ESI): 683.20 [M+H]⁺.

### Synthesis of N-((S)-1-(((S)-1-((4-((((19S,22R,23R,23Ar,25S,27Ar,29R,210R,210Ar,212R,214Ar,39S,E)-23,210-difluoro-212-mercapto-25,212-dioxido-23,23^{a},27^{a},29,210,210^{a},214,214^{a}-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-en-25-yl)thio)methyl)phenyl)amino)-1-oxo-5-ureidopentan-2-yl)amino)-3-methyl-1-oxobutan-2-yl)-6-(2,5-dioxo-2,5-dihydro-1H-pyrrol-1-yl)hexanamide (LP34)

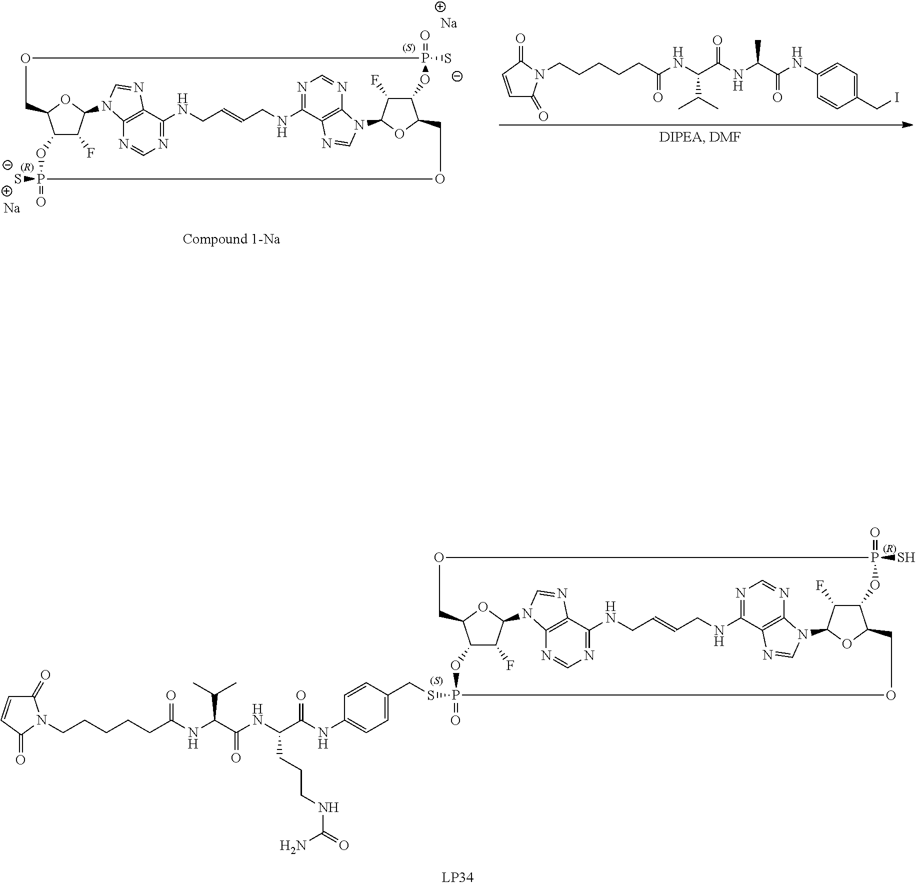

A solution of (19S,22R,23R,23Ar,25R,27Ar,29R,210R,210Ar,212S,214Ar,39S,E)-23,210-difluoro-25,212-dimercapto-23,23a,27a,29,210,210a,214,214a-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-ene 25,212-dioxide, disodium salt (50 mg, 0.063 mmol) and 6-(2,5-dioxo-2,5-dihydro-1H-pyrrol-1-yl)-N-((S)-1-(((S)-1-((4-(iodomethyl)phenyl)amino)-1-oxo-5-ureidopentan-2-yl)amino)-3-methyl-1-oxobutan-2-yl)hexanamide (65.6 mg, 0.053 mmol) in DMF (2.5 ml) was stirred at rt in dark overnight. The resulting solution was purified by Prep-HPLC, which resulted in 10.4 mg of target product LP34.

LC-MS (ESI): 1301.38 [M+H]⁺.

### 34. Synthesis of LP35

The synthesis of LP35 is shown below:

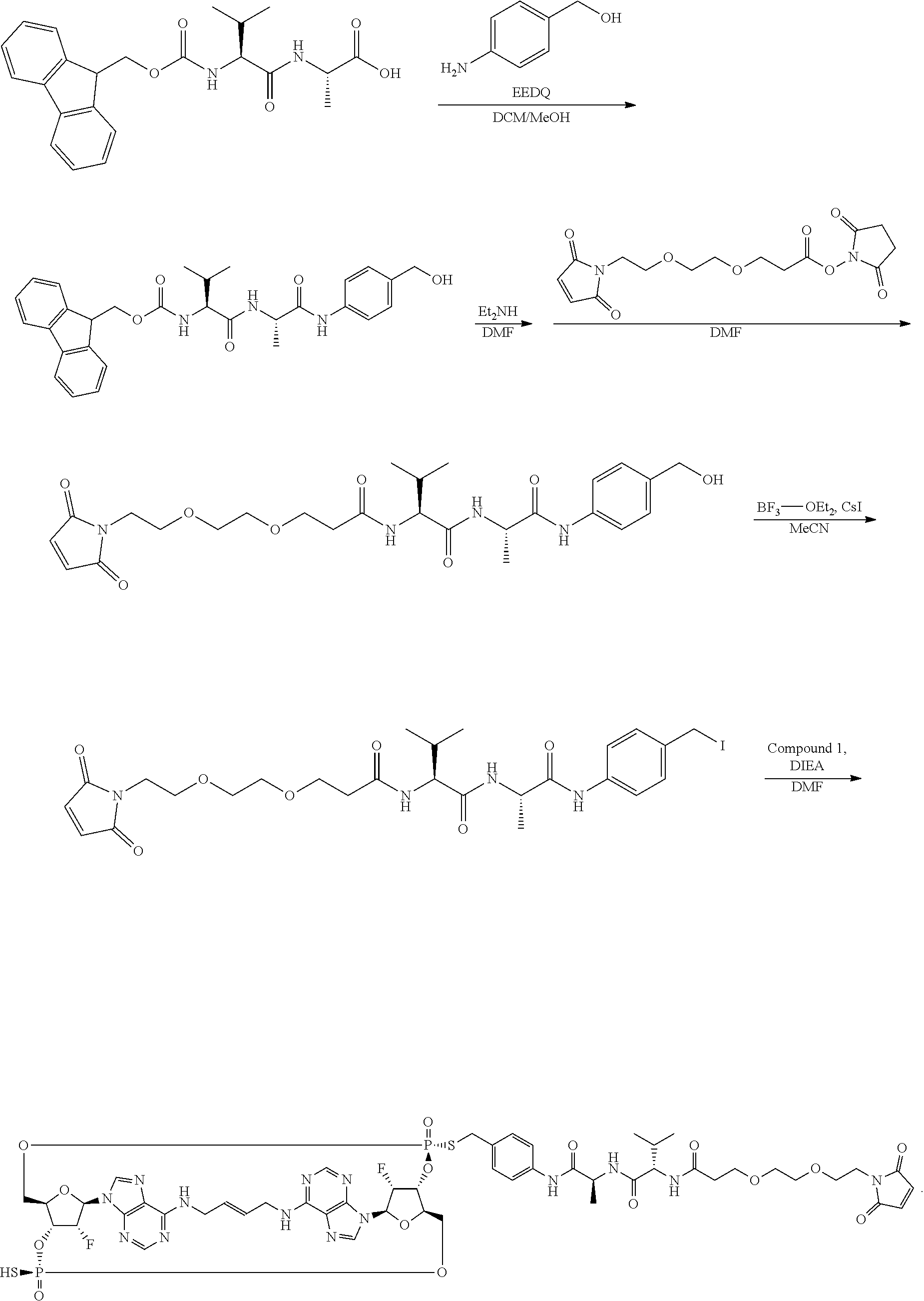

### Synthesis of (9H-fluoren-9-yl)methyl N-[(1S)-1-{[(1S)-1-{[4-(hydroxymethyl)phenyl]carbamoyl}ethyl]carbamoyl}-2-methylpropyl]carbamate

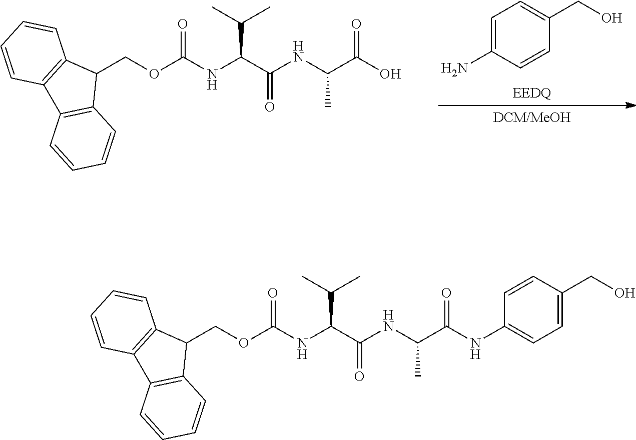 [SMILES: CC(C)[C@H](NC(=O)OCC1c2ccccc2-c2ccccc21)C(=O)N[C@@H](C)C(=O)O ; Nc1ccc(CO)cc1 ; CC(C)[C@H](NC(=O)OCC1c2ccccc2-c2ccccc21)C(=O)N[C@@H](C)C(=O)Nc1ccc(CO)cc1]

To a solution of (2S)-2-[(2S)-2-({[(9H-fluoren-9-yl)methoxy]carbonyl}amino)-3-methylbutanamido]propanoic acid (5.53 g, 13.472 mmol) in DCM (150 ml) and MeOH (20 ml) under dark, 4-aminobenzyl alcohol (1.991 g, 16.167 mmol) and 1-Ethoxycarbonyl-2-ethoxy-1,2-dihydroquinoline (6.66 g, 26.945 mmol) were added portionwise. The mixture was stirred at room temperature for 16 hours. Precipitate was collected and was washed with DCM/MeOH (200 mL, 9/1) and MTBE (100 mL) to give (9H-fluoren-9-yl)methyl N-[(1S)-1-{[(1S)-1-{[4-(hydroxymethyl)phenyl]carbamoyl}ethyl]carbamoyl}-2-methylpropyl]carbamate (4.57 g). ¹H NMR (400 MHz, DMSO-d6) δ ppm 0.87 (br d, J=6.78 Hz, 3H) 0.90 (br d, J=6.78 Hz, 3H) 1.31 (d, J=7.03 Hz, 3H) 1.94-2.06 (m, 1H) 3.92 (br t, J=7.97 Hz, 1H) 4.17-4.35 (m, 3H) 4.44 (br d, J=5.52 Hz, 3H) 5.11 (t, J=5.65 Hz, 1H) 7.24 (d, J=8.41 Hz, 2H) 7.29-7.37 (m, 2H) 7.38-7.49 (m, 3H) 7.54 (d, J=8.28 Hz, 2H) 7.75 (br t, J=7.15 Hz, 2H) 7.90 (d, J=7.40 Hz, 2H) 8.18 (br d, J=7.03 Hz, 1H), 9.93 (s, 1H)

### Synthesis of (2S)-2-(3-{2-[2-(2,5-dioxo-2,5-dihydro-1H-pyrrol-1-yl)ethoxy]ethoxy}propanamido)-N-[(1S)-1-{[4-(hydroxymethyl)phenyl]carbamoyl}ethyl]-3-methylbutanamide

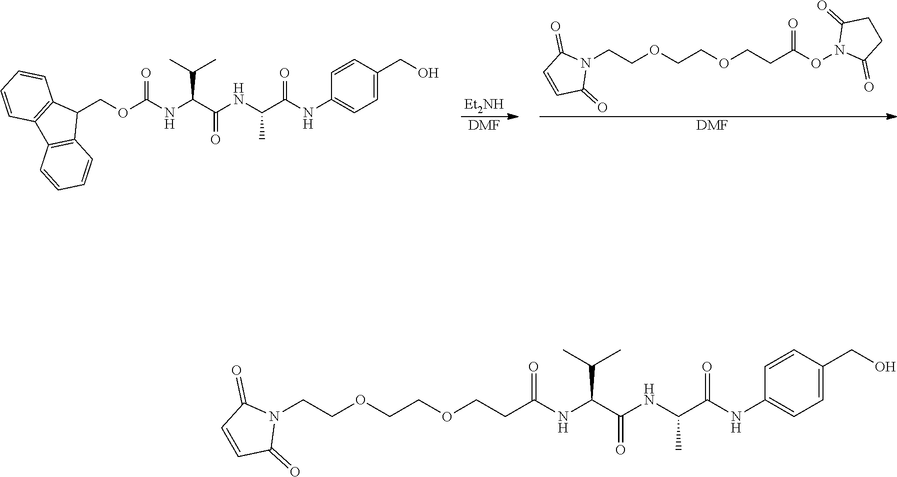

To a solution of (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (382 mg, 0.741 mmol) in DMF (10 ml) was added diethylamine (0.369 ml, 3.528 mmol). The reaction mixture was stirred at room temperature for 1 hour. Solvent was removed under reduced pressure. Obtained residue was dissolved in DMF (10 ml) and was added 2,5-dioxopyrrolidin-1-yl 3-{2-[2-(2,5-dioxo-2,5-dihydro-1H-pyrrol-1-yl)ethoxy]ethoxy}propanoate (250 mg, 0.706 mmol). The reaction was stirred at room temperature for 16 hours. Solvent was removed in vacuo, and obtained residue was purified by silica gel chromatography to give (2S)-2-(3-{2-[2-(2,5-dioxo-2,5-dihydro-1H-pyrrol-1-yl)ethoxy]ethoxy}propanamido)-N-[(1S)-1-{[4-(hydroxymethyl)phenyl]carbamoyl}ethyl]-3-methylbutanamide (117 mg). ¹H NMR (400 MHz, DMSO-d6) δ ppm 0.84 (br d, J=6.78 Hz, 3H) 0.88 (br d, J=6.78 Hz, 3H) 1.31 (d, J=7.15 Hz, 3H) 1.90-2.02 (m, 1H) 2.32-2.48 (m, 2H) 3.40-3.60 (m, 10H) 4.21 (dd, J=8.34, 6.96 Hz, 1H) 4.36-4.45 (m, 3H) 5.10 (t, J=5.71 Hz, 1H) 7.03 (s, 2H) 7.24 (d, J=8.41 Hz, 2H) 7.54 (d, J=8.53 Hz, 2H) 7.87 (br d, J=8.66 Hz, 1H) 8.16 (br d, J=7.03 Hz, 1H) 9.84 (s, 1H).

### Synthesis of (2S)-2-(3-{2-[2-(2,5-dioxo-2,5-dihydro-1H-pyrrol-1-yl)ethoxy]ethoxy}propanamido)-N-[(1S)-1-{[4-(iodomethyl)phenyl]carbamoyl}ethyl]-3-methylbutanamide

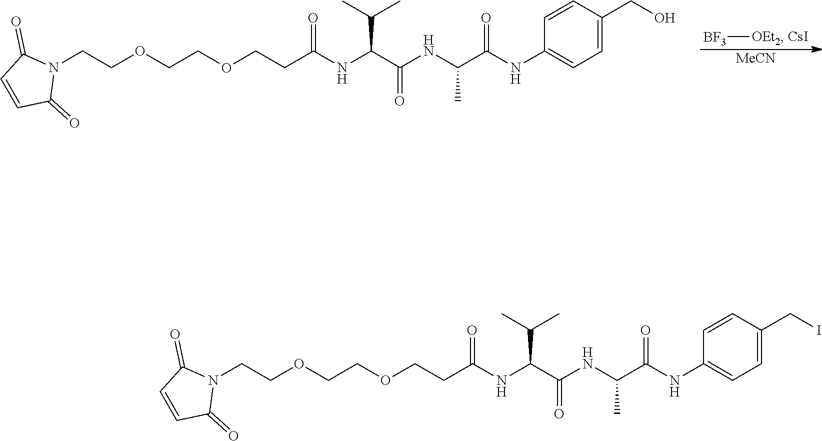

To a solution of (S)-2-(3-(2-(2-(2,5-dioxo-2,5-dihydro-1H-pyrrol-1-yl)ethoxy)ethoxy)propanamido)-N-((S)-1-((4-(hydroxymethyl)phenyl)amino)-1-oxopropan-2-yl)-3-methylbutanamide (70 mg, 0.131 mmol) in acetonitrile (2 ml, 38.292 mmol) was added borontrifluoride-ether complex (0.021 ml, 0.171 mmol) and then cesium iodide (41.0 mg, 0.158 mmol) at room temperature. The mixture was stirred at room temperature for 16 hours. The reaction mixture was diluted with DCM (50 mL), and the organic layer was washed with H₂O (10 mL) and sat. NaHCO₃ aq. (10 mL). The combined aqueous layers were extracted with DCM (15 ml×2).

Then, combined organic layers were washed with 5% NaHSO₃ aq. and was dried over Na₂SO₄. Solid was filtered out and solvent was removed under reduced pressure. Obtained crude material (66.4 mg) was used to the next step without further purification.

### Synthesis of (2S)-N-[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]-2-(3-{2-[2-(2,5-dioxo-2,5-dihydro-1H-pyrrol-1-yl)ethoxy]ethoxy}propanamido)-3-methylbutanamide (LP35)

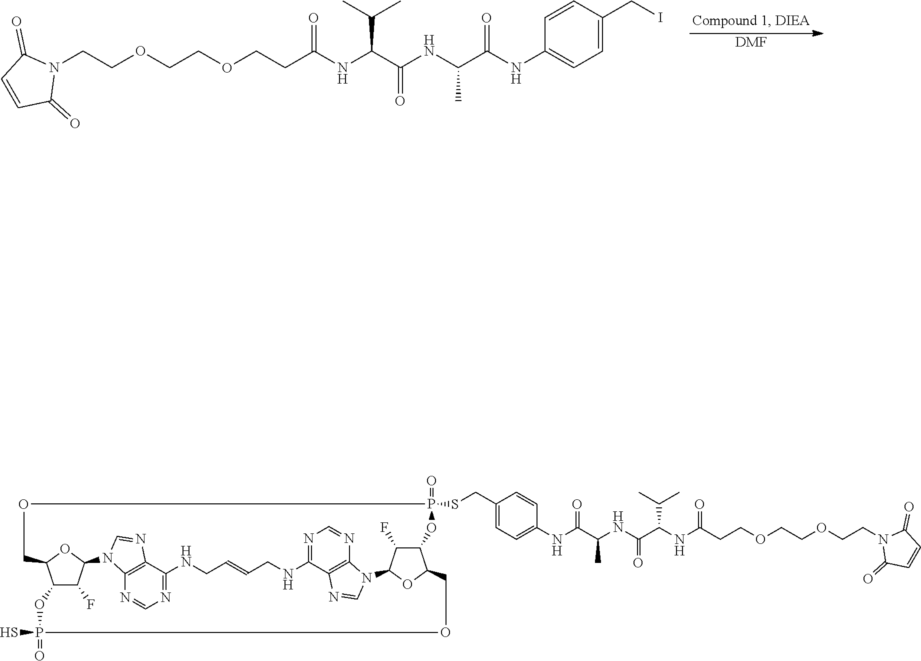

To a suspension of Compound 1 (70 mg, 0.094 mmol) and DIPEA (0.066 ml, 0.375 mmol) in DMF (2 ml) at 0° C., crude (S)-2-(3-(2-(2-(2,5-dioxo-2,5-dihydro-1H-pyrrol-1-yl)ethoxy)ethoxy)propanamido)-N-((S)-1-((4-(iodomethyl)phenyl)amino)-1-oxopropan-2-yl)-3-methylbutanamide (60.2 mg) in DMF (2 ml) was added. The reaction mixture was allowed to warm up to room temperature and was stirred for 16 hours. Solvent was removed under reduced pressure, and obtained crude material was purified by reverse phase HPLC to give (2S)-N-[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]-2-(3-{2-[2-(2,5-dioxo-2,5-dihydro-1H-pyrrol-1-yl)ethoxy]ethoxy}propanamido)-3-methylbutanamide (LP35) (14.6 mg).

ESI [M+H]⁺ 1261.03.

### 35. Synthesis of LP36

The synthesis of LP36 is shown below:

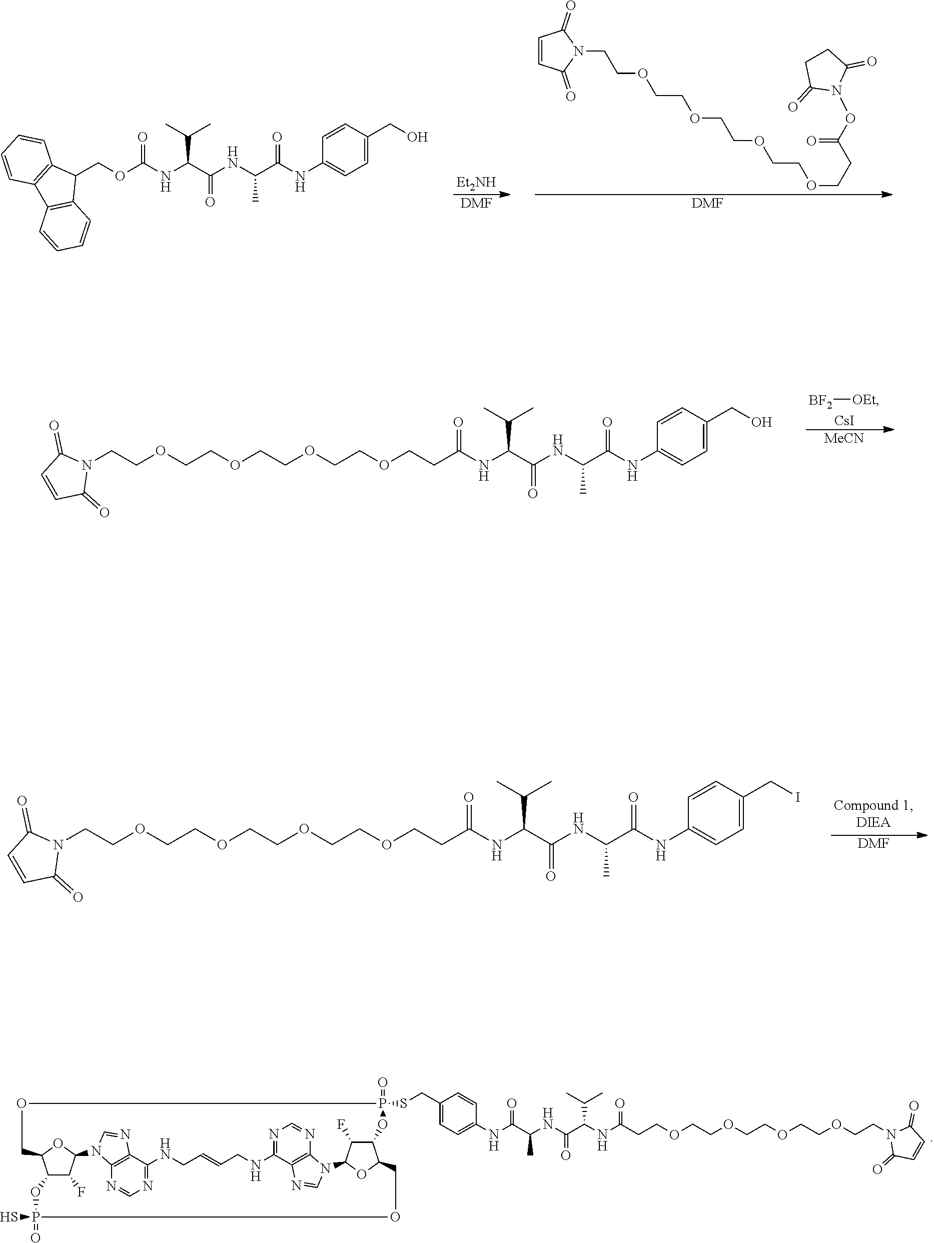

### Synthesis of 1-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]-3,6,9,12-tetraoxapentadecan-15-amide

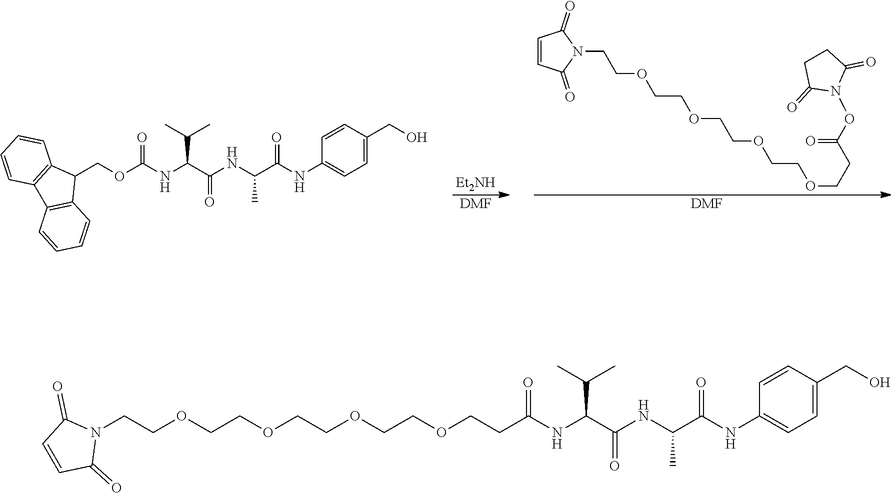

To a solution of (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (306 mg, 0.593 mmol) in DMF (10 ml) was added diethylamine (0.295 ml, 2.825 mmol). The reaction mixture was stirred at room temperature for 1 hour and then solvent was removed under reduced pressure. Obtained residue was dissolved in DMF (10 ml) and 2,5-dioxopyrrolidin-1-yl 1-(2,5-dioxo-2,5-dihydro-1H-pyrrol-1-yl)-3,6,9,12-tetraoxapentadecan-15-oate (250 mg, 0.565 mmol) was added and the reaction mixture was stirred at room temperature for 16 hours. Solvent was removed under reduced pressure, and obtained residue was purified by silica gel chromatography to give a product (223 mg). ¹H NMR (400 MHz, DMSO-d6) δ ppm 0.84 (br d, J=6.78 Hz, 3H) 0.88 (br d, J=6.65 Hz, 3H) 1.31 (br d, J=7.03 Hz, 3H) 1.91-2.03 (m, 1H) 2.33-2.48 (m, 2H) 3.42-3.62 (m, 18H) 4.21 (br dd, J=8.47, 6.84 Hz, 1H) 4.35-4.46 (m, 3H) 5.10 (t, J=5.65 Hz, 1H) 7.03 (s, 2H) 7.24 (d, J=8.41 Hz, 2H) 7.54 (d, J=8.41 Hz, 2H) 7.89 (br d, J=8.53 Hz, 1H) 8.16 (br d, J=6.90 Hz, 1H) 9.84 (s, 1H).

### Synthesis of 1-(2,5-dioxo-2,5-dihydro-1H-pyrrol-1-yl)-N-[(1S)-1-{[(1S)-1-{[4-(iodomethyl)phenyl]carbamoyl}ethyl]carbamoyl}-2-methylpropyl]-3,6,9,12-tetraoxapentadecan-15-amide

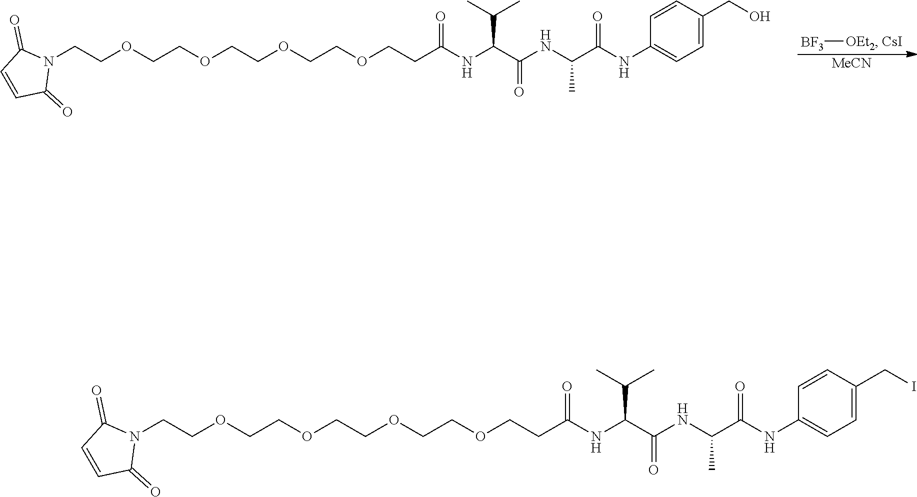

To a solution of 1-(2,5-dioxo-2,5-dihydro-1H-pyrrol-1-yl)-N-[(1S)-1-{[(1S)-1-{[4-(hydroxymethyl)phenyl]carbamoyl}ethyl]carbamoyl}-2-methylpropyl]-3,6,9,12-tetraoxapentadecan-15-amide (80 mg, 0.129 mmol) in acetonitrile (2 ml) was added Borontrifluoride ether complex (0.021 ml, 0.168 mmol) and then Cesium iodide (40.2 mg, 0.155 mmol) portionwise at room temperature. The reaction mixture was stirred at room temperature for 16 hours then diluted with DCM (50 mL). The organic layer was washed with H₂O (10 mL) and sat. NaHCO₃ aq. (10 mL), and combined aqueous layers were extracted with DCM (15 ml×2). The combined organic layers were washed with 5% NaHSO₃ aq. and was dried over Na₂SO₄. Solid was removed with filter and solvent was removed under reduced pressure. The crude material was used to the next step without further purification.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-1-(2,5-dioxo-2,5-dihydro-1H-pyrrol-1-yl)-3,6,9,12-tetraoxapentadecan-15-amide (LP36)

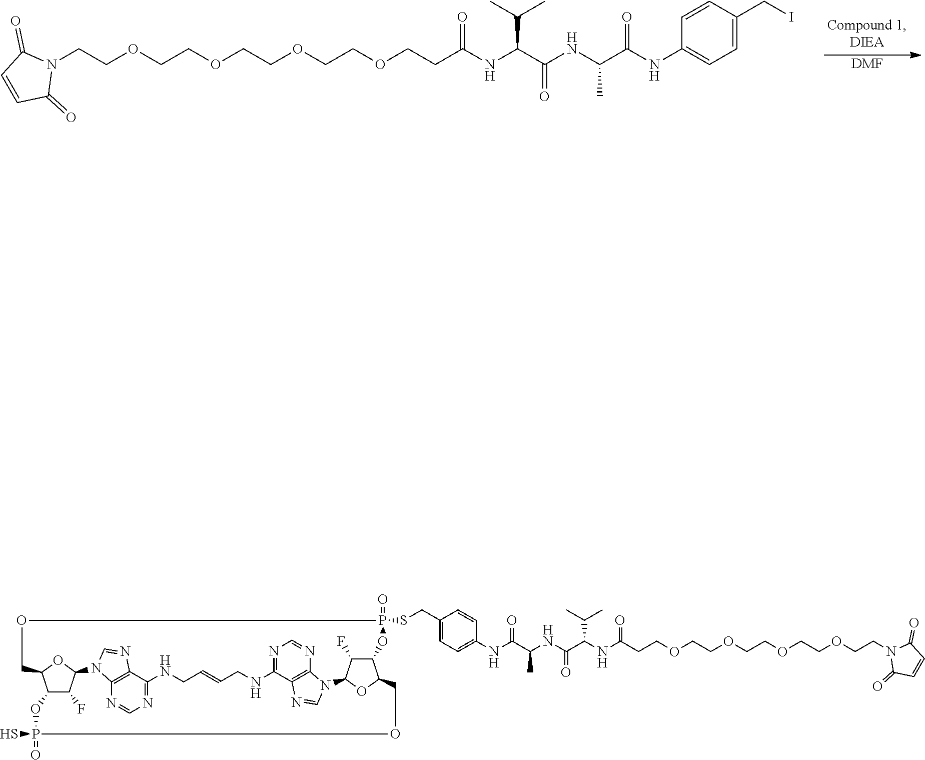

To a solution of Compound 1 (70 mg, 0.094 mmol) and DIPEA (0.066 ml, 0.375 mmol) in DMF (2 ml) at 0° C., 1-(2,5-dioxo-2,5-dihydro-1H-pyrrol-1-yl)-N-[(1S)-1-{[(1S)-1-{[4-(iodomethyl)phenyl]carbamoyl}ethyl]carbamoyl}-2-methylpropyl]-3,6,9,12-tetraoxapentadecan-15-amide (68.5 mg, 0.075 mmol) in DMF (2 ml) was added. The reaction mixture was allowed to warm up to room temperature and was stirred for 16 hours. Solvent was removed under reduced pressure and obtained crude material was purified by reverse phase HPLC to give LP36 (14.0 mg).

ESI [M+H]⁺: 1349.18.

### 36. Synthesis of LP37

The synthesis of LP37 is shown below:

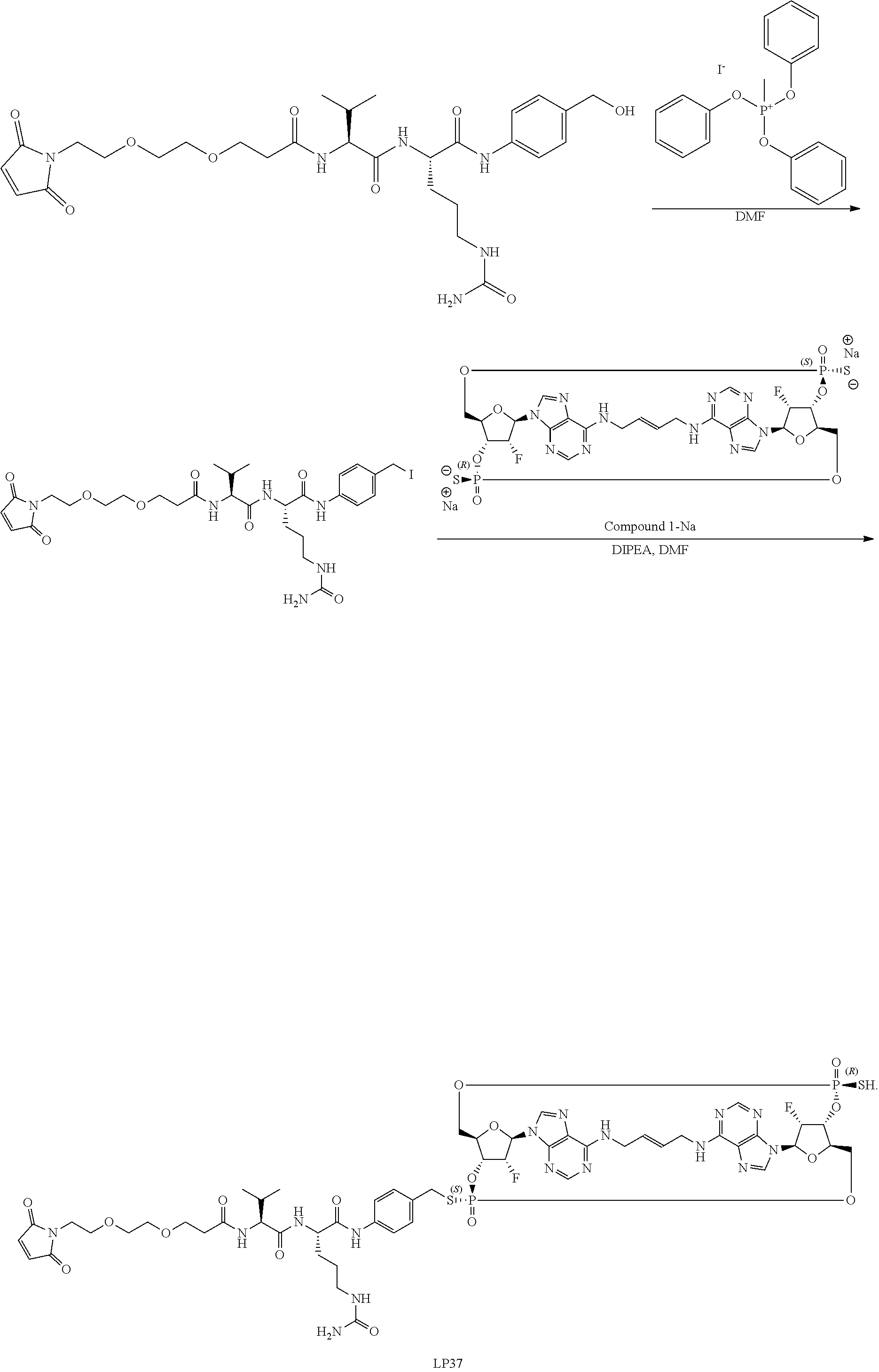

### Synthesis of (S)-2-((S)-2-(3-(2-(2-(2,5-dioxo-2,5-dihydro-1H-pyrrol-1-yl)ethoxy)ethoxy)propanamido)-3-methylbutanamido)-N-(4-(iodomethyl)phenyl)-5-ureidopentanamide

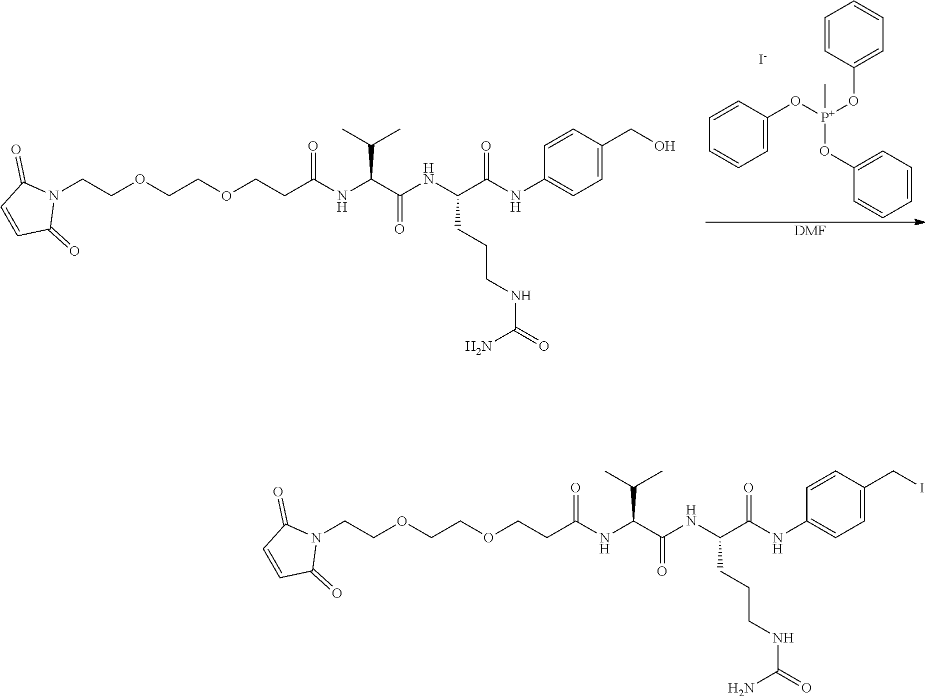

To a solution of (S)-2-((S)-2-(3-(2-(2-(2,5-dioxo-2,5-dihydro-1H-pyrrol-1-yl)ethoxy)ethoxy)propanamido)-3-methylbutanamido)-N-(4-(hydroxymethyl)phenyl)-5-ureidopentanamide (50 mg, 0.081 mmol) in DMF (1 mL) at rt was added methyltriphenoxyphosphonium iodide (110 mg, 0.242 mmol). The resulting mixture was stirred in dark at rt for 5 h. The reaction mixture was diluted with 20 mL EtOAc and washed with a sat'd solution of Na₂S₂O₃ (5 mL). The aqueous layer was extracted with EtOAc (10 mL). The combined organic layers were washed with 30% NaCl aqueous solution (5 mL), dried over Na₂SO₄, filtered and concentrated in vacuo. The resulting solid was further dried in vacuo and used in next step without further purification. LC-MS (ESI): 729.32 [M+H]⁺.

### Synthesis of (S)-N-(4-((((19S,22R,23R,23aR,25S,27aR,29R,210R,210aR,212R,214aR,39S,E)-23,210-difluoro-212-mercapto-25,212-dioxido-23,23a,27a,29,210,210a,214,214a-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-en-25-yl)thio)methyl)phenyl)-2-((S)-2-(3-(2-(2-(2,5-dioxo-2,5-dihydro-1H-pyrrol-1-yl)ethoxy)ethoxy)propanamido)-3-methylbutanamido)-5-ureidopentanamide (LP37)

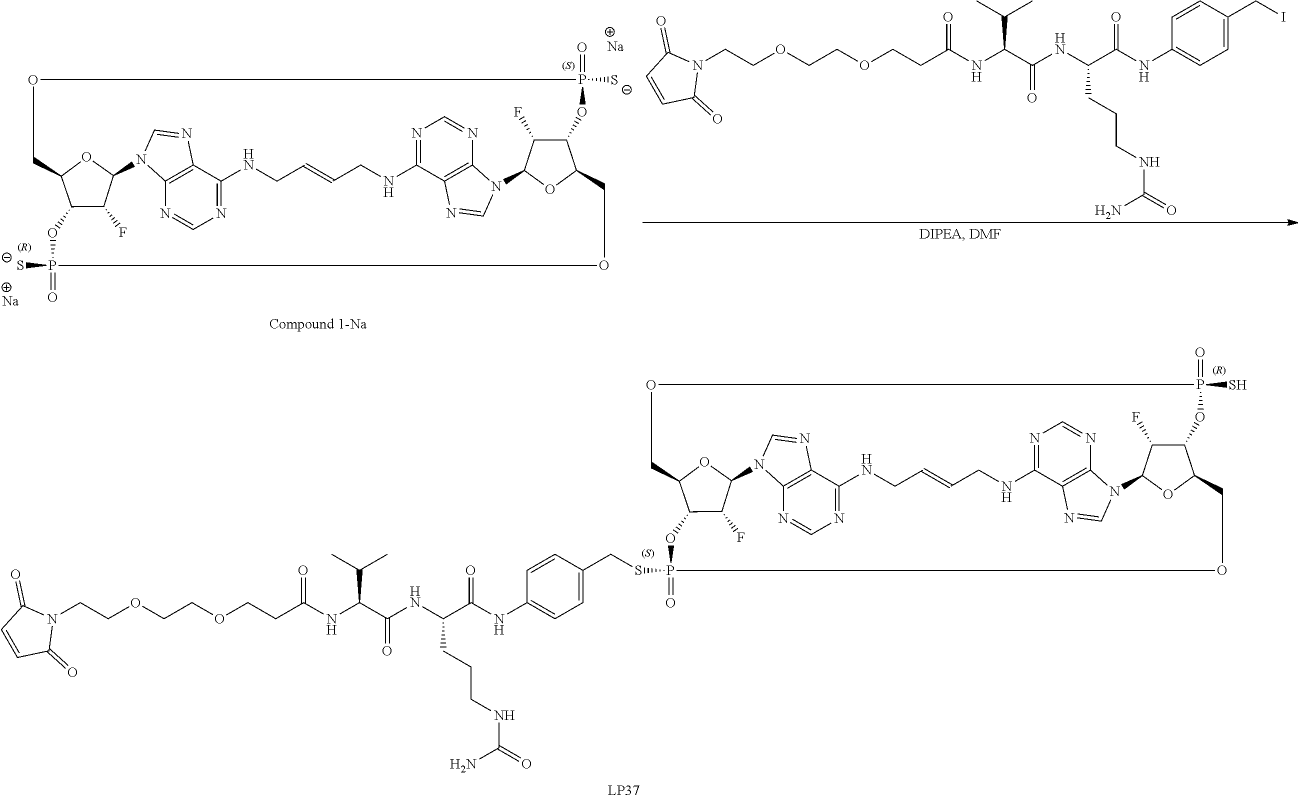

A solution of (19S,22R,23R,23aR,25R,27aR,29R,210R,210aR,212S,214aR,39S,E)-23,210-difluoro-25,212-dimercapto-23,23a,27a,29,210,210a,214,214a-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-ene 25,212-dioxide, disodium salt (70 mg, 0.088 mmol) and (S)-2-((S)-2-(3-(2-(2-(2,5-dioxo-2,5-dihydro-1H-pyrrol-1-yl)ethoxy)ethoxy)propanamido)-3-methylbutanamido)-N-(4-(iodomethyl)phenyl)-5-ureidopentanamide (57.2 mg, 0.071 mmol) in DMF (2.5 ml) was stirred at rt in dark for 30 min. The resulting solution was kept at −20° C. over weekend, warmed to rt and treated with 40 mL EtoAc. The resulting solid was collected by filtration, rinsed with EtOAc and dried in vacuo. The resulting solid was further purified by Prep-HPLC, which resulted in 8.7 mg of target product LP37.

LC-MS (ESI): 1347.28 [M+H]⁺.

### 37. Synthesis of LP38

The synthesis of LP38 is shown below:

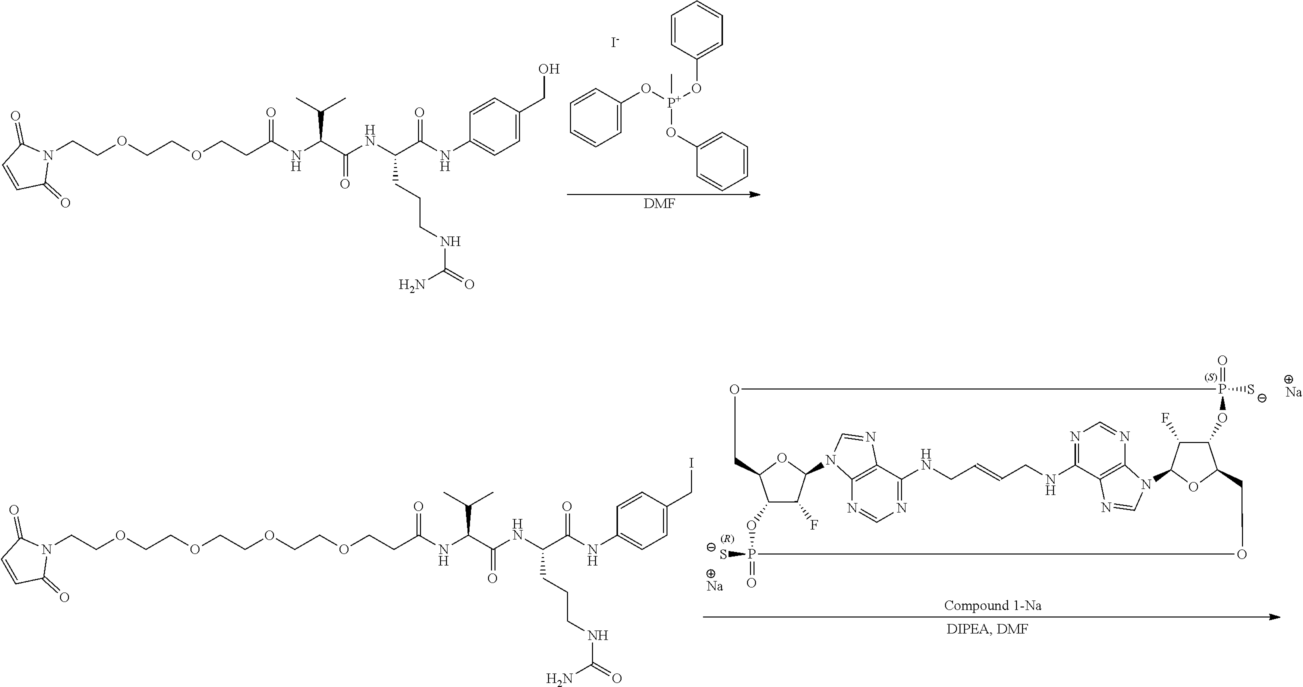

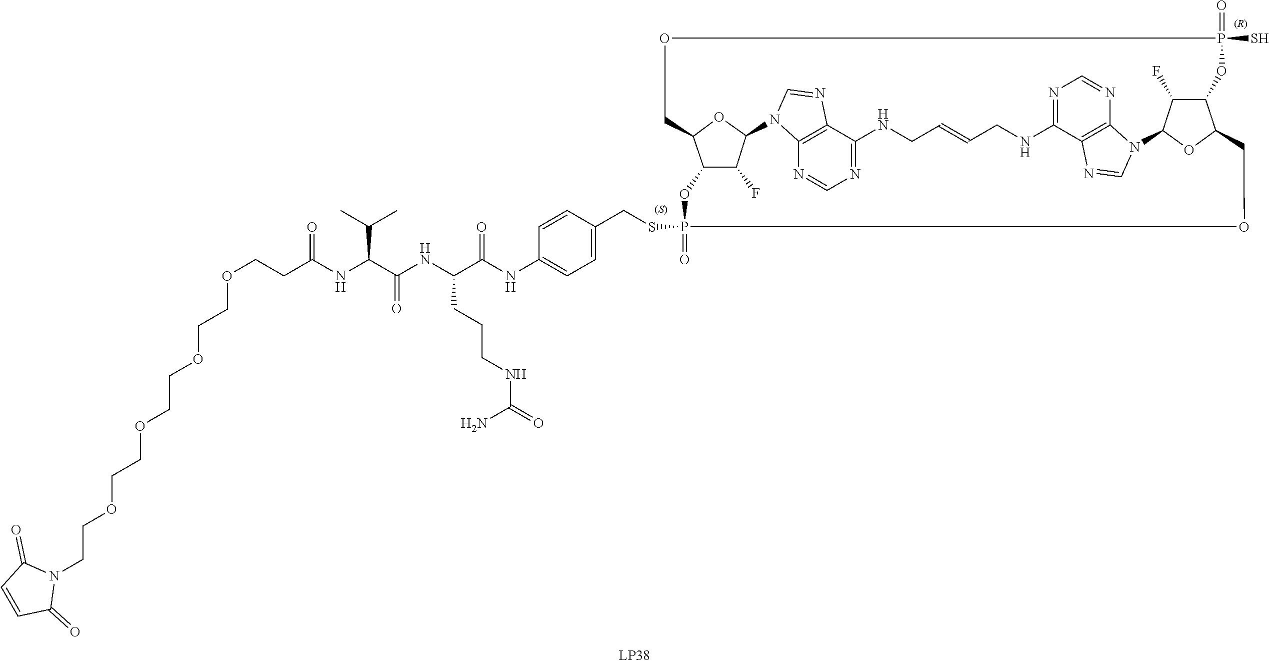

### Synthesis of 1-(2,5-dioxo-2,5-dihydro-1H-pyrrol-1-yl)-N-((S)-1-(((S)-1-((4-(iodomethyl)phenyl)amino)-1-oxo-5-ureidopentan-2-yl)amino)-3-methyl-1-oxobutan-2-yl)-3,6,9,12-tetraoxapentadecan-15-amide

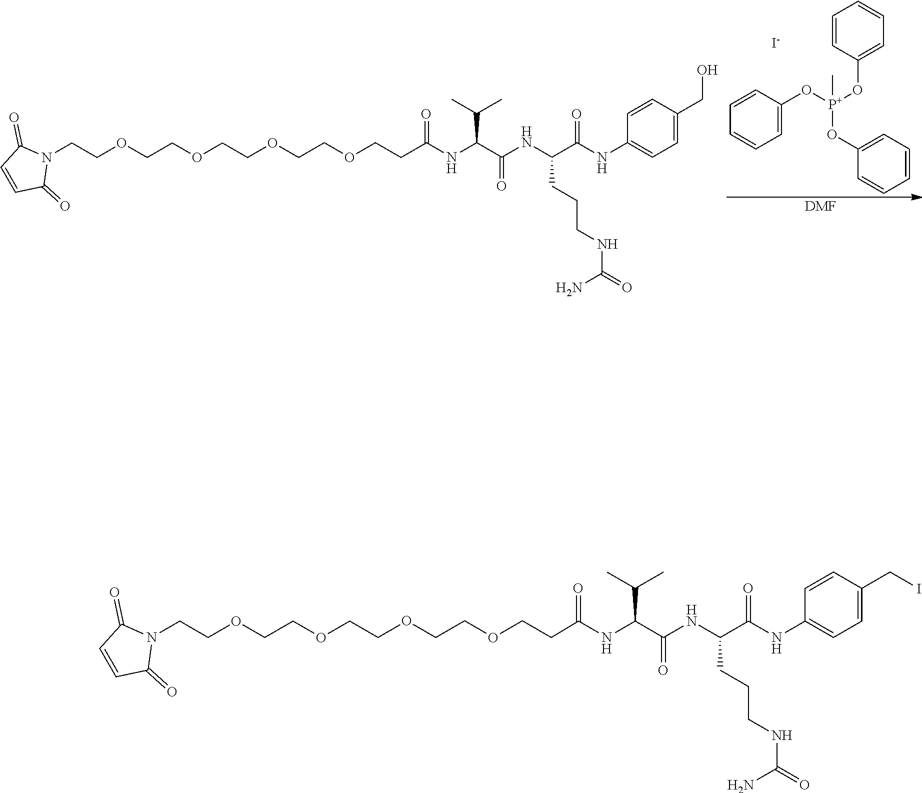

To a solution of 1-(2,5-dioxo-2,5-dihydro-1H-pyrrol-1-yl)-N-((S)-1-(((S)-1-((4-(hydroxymethyl)phenyl)amino)-1-oxo-5-ureidopentan-2-yl)amino)-3-methyl-1-oxobutan-2-yl)-3,6,9,12-tetraoxapentadecan-15-amide (70 mg, 0.099 mmol) in DMF (1 ml) at rt was added methyltriphenoxyphosphonium iodide (134 mg, 0.297 mmol). The resulting mixture was stirred in dark at rt overnight. The reaction mixture was diluted with 15 mL DCM and washed with a sat'd solution of Na₂S₂O₃ (5 mL). The aqeous layer was extracted with DCM (10 mL). The combined organic layers were washed with 30% NaCl aqeous solution (5 mL), dried over Na₂SO₄, filtered and concentrated in vacuo. The resulting solid was further dried in vacuo and used in next step without further purification. LC-MS (ESI): 817.22 [M+H]⁺.

### Synthesis of N-((S)-1-(((S)-1-((4-((((19S,22R,23R,23aR,25S,27aR,29R,210R,210aR,212R,214aR,39S,E)-23,210-difluoro-212-mercapto-25,212-dioxido-23,23a,27a,29,210,210a,214,214a-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-en-25-yl)thio)methyl)phenyl)amino)-1-oxo-5-ureidopentan-2-yl)amino)-3-methyl-1-oxobutan-2-yl)-1-(2,5-dioxo-2,5-dihydro-1H-pyrrol-1-yl)-3,6,9,12-tetraoxapentadecan-15-amide (LP38)

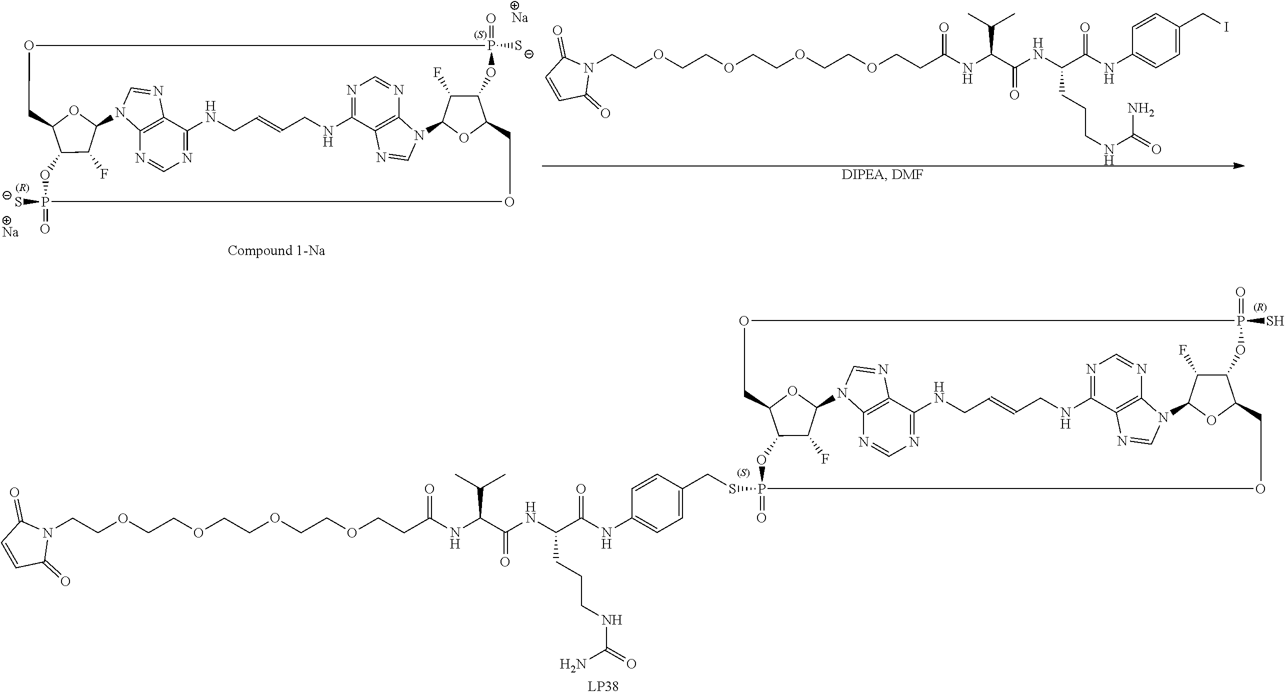

A solution of (19S,22R,23R,23aR,25R,27aR,29R,210R,210aR,212S,214aR,39S, E)-23,210-difluoro-25,212-dimercapto-23,23a,27a,29,210,210a,214,214a-octahydro-19H,22H,27H,39H-4,9-diaza-1,3(9,6)-dipurina-2(2,9)-difuro[3,2-d:3′,2′-j][1,3,7,9]tetraoxa[2,8]diphosphacyclododecinacyclononaphan-6-ene 25,212-dioxide, disodium salt (80 mg, 0.101 mmol) and 1-(2,5-dioxo-2,5-dihydro-1H-pyrrol-1-yl)-N-((S)-1-(((S)-1-((4-(iodomethyl)phenyl)amino)-1-oxo-5-ureidopentan-2-yl)amino)-3-methyl-1-oxobutan-2-yl)-3,6,9,12-tetraoxapentadecan-15-amide (82 mg, 0.10 mmol) in DMF (2.5 ml) was stirred at rt in dark for 1 h. The resulting solution was treated with 30 mL EtOAc. The resulting solid was collected by filtration, rinsed with EtOAc and dried in vacuo. The resulting solid was further purified by Prep-HPLC, which resulted in 13.5 mg of target product LP38.

LC-MS (ESI): 1435.32 [M+H]⁺.

### 38. Synthesis of LP39

The synthesis of LP39 is shown below:

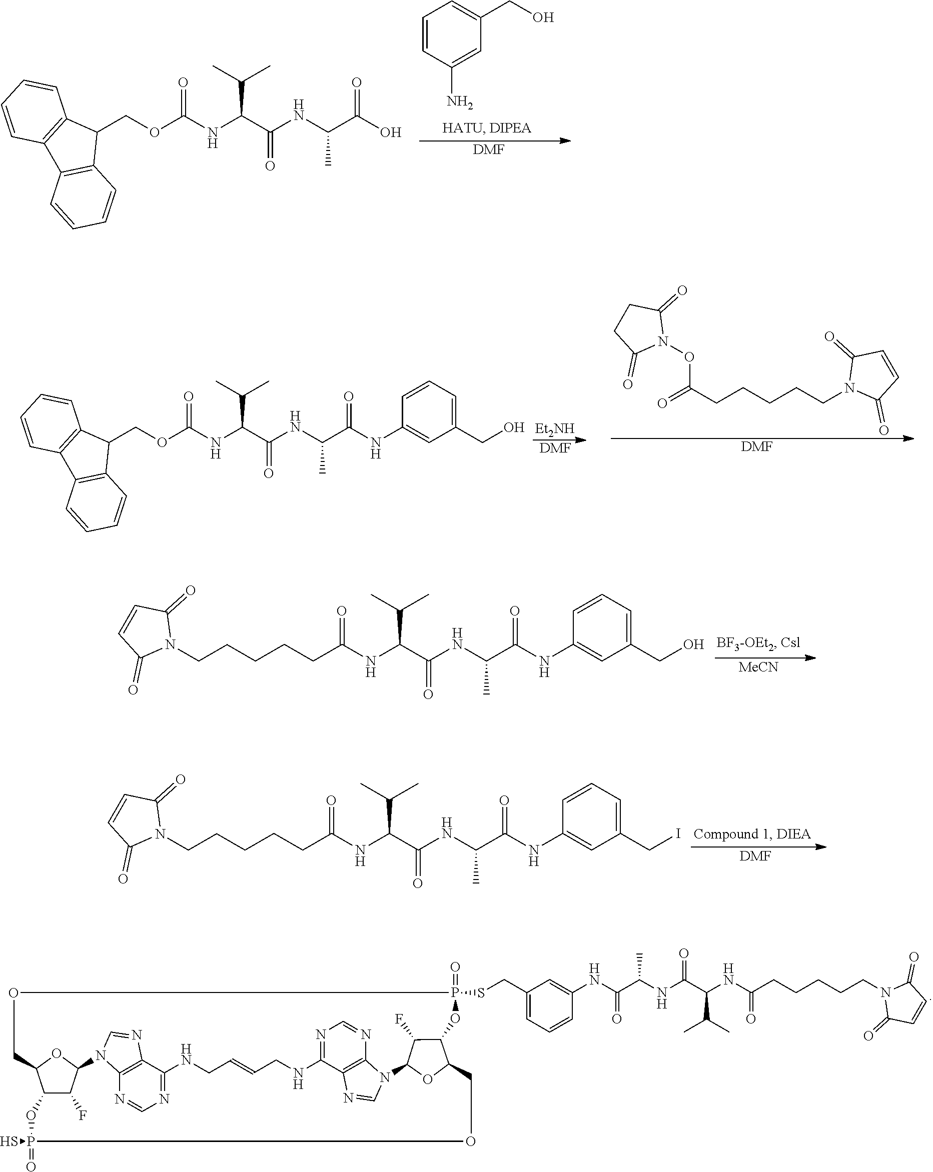

### Synthesis of (9H-fluoren-9-yl)methyl N-[(1S)-1-{[(1S)-1-{[3-(hydroxymethyl)phenyl]carbamoyl}ethyl]carbamoyl}-2-methylpropyl]carbamate

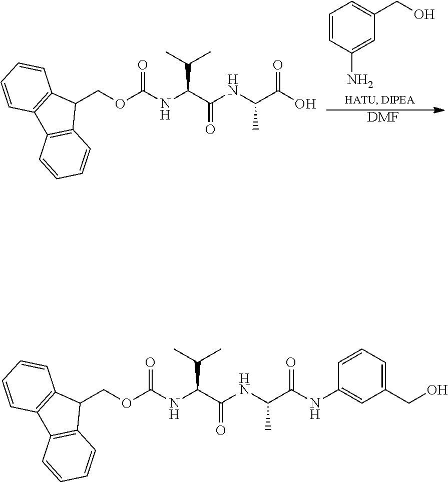

To a solution of Fmoc-Val-Ala-OH (400 mg, 0.974 mmol) and (3-aminophenyl)methanol (180 mg, 1.462 mmol) in DMF (10 mL) was added DIPEA (0.851 mL, 4.872 mmol) and HATU (741 mg, 1.949 mmol). The reaction mixture was stirred at room temperature for 2 hours and then excess DMF was removed under reduced pressure. Obtained residue was added water (50 mL) to give a precipitate. Precipitate was collected, washed with H₂O and MTBE to give a product as a light brown solid (318 mg).

### Synthesis of 6-(2,5-dioxo-2,5-dihydro-1H-pyrrol-1-yl)-N-[(1S)-1-{[(1S)-1-{[3-(hydroxymethyl)phenyl]carbamoyl}ethyl]carbamoyl}-2-methylpropyl]hexanamide

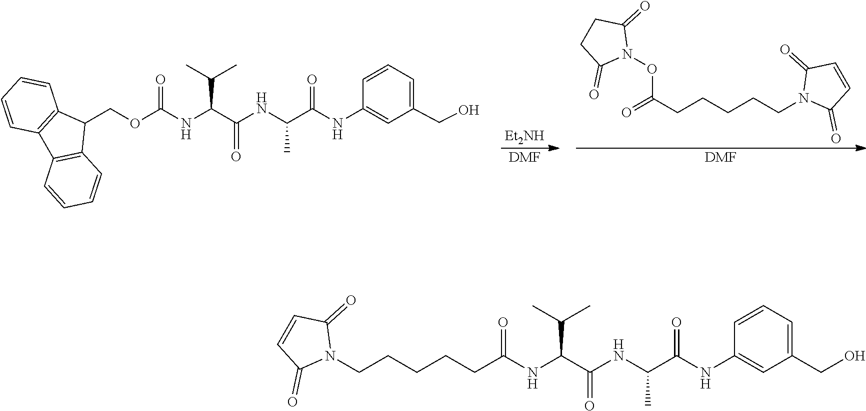

To a solution of (9H-fluoren-9-yl)methyl ((S)-1-(((S)-1-((3-(hydroxymethyl)phenyl)amino)-1-oxopropan-2-yl)amino)-3-methyl-1-oxobutan-2-yl)carbamate (299 mg, 0.579 mmol) in DMF (6 ml) was added diethylamine (0.288 ml, 2.757 mmol). The reaction mixture was stirred at room temperature for 1 hour, and then solvent was removed under reduced pressure. Obtained residue was dissolved in DMF (6 ml) and 2,5-dioxopyrrolidin-1-yl 6-(2,5-dioxo-2,5-dihydro-1H-pyrrol-1-yl)hexanoate (170 mg, 0.551 mmol) was added and stirred for 2 hours at room temperature. Solvent was removed under reduced pressure, and obtained residue was purified by reverse phase silica gel chromatography (H₂O/MeCN/HCOOH=95/5/0.1 to 50/50/0.1) to give a product (85.1 mg).

### Synthesis of 6-(2,5-dioxo-2,5-dihydro-1H-pyrrol-1-yl)-N-[(1S)-1-{[(1S)-1-{[3-(iodomethyl)phenyl]carbamoyl}ethyl]carbamoyl}-2-methylpropyl]hexanamide

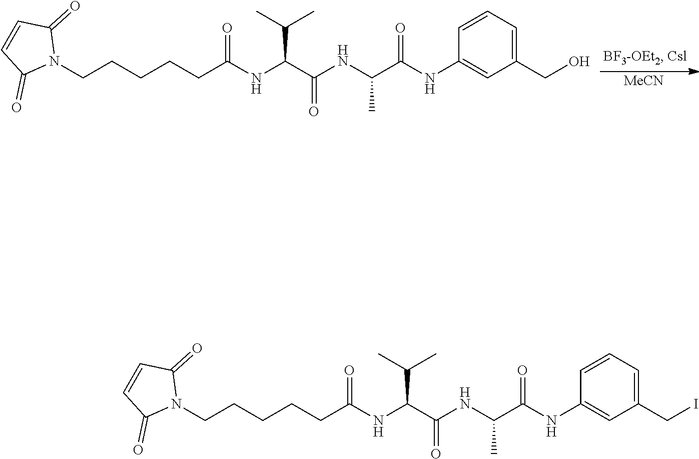 [SMILES: "MeCN" ; CC(C)[C@H](NC(=O)CCCCCN1C(=O)C=CC1=O)C(=O)N[C@@H](C)C(=O)Nc1cccc(CO)c1 ; CC(C)[C@H](NC(=O)CCCCCN1C(=O)C=CC1=O)C(=O)N[C@@H](C)C(=O)Nc1cccc(CI)c1]

To a solution of 6-(2,5-dioxo-2,5-dihydro-1H-pyrrol-1-yl)-N-((S)-1-(((S)-1-((3-(hydroxymethyl)phenyl)amino)-1-oxopropan-2-yl)amino)-3-methyl-1-oxobutan-2-yl)hexanamide (86.1 mg, 0.177 mmol) in acetonitrile (2 ml) was added Borontrifluoride ether complex (0.028 ml, 0.23 mmol) and then Cesium iodide (55.2 mg, 0.212 mmol) at room temperature. The mixture was stirred at room temperature for 16 hours, and then was diluted with DCM (50 mL). The organic layer was washed with H₂O (10 mL) and sat. NaHCO₃ aq. (10 mL). Combined aqueous layers were extracted with DCM (15 ml×2), and combined organic layers were washed with 5% NaHSO₃ aq. and was dried over Na₂SO₄. Solid was removed with filter and solvent was removed under reduced pressure. Obtained crude material was used in the next step without further purification.

### Synthesis of N-[(1S)-1-{[(1S)-1-{[3-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP39)

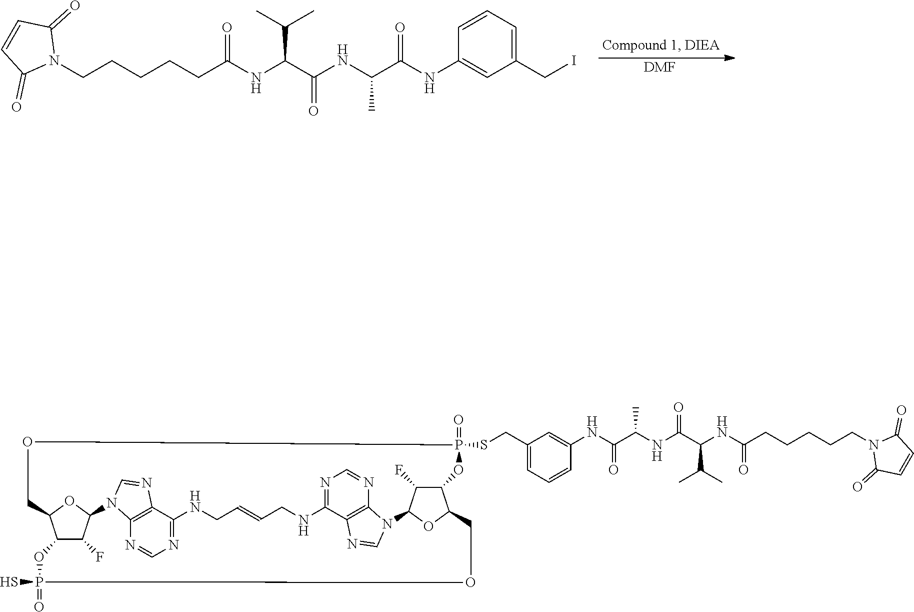

To a suspension of Compound 1 (50 mg, 0.067 mmol) and DIPEA (0.047 ml, 0.268 mmol) in DMF (2 ml, 25.829 mmol) at 0° C., 6-(2,5-dioxo-2,5-dihydro-1H-pyrrol-1-yl)-N-[(1S)-1-{[(1S)-1-{[3-(iodomethyl)phenyl]carbamoyl}ethyl]carbamoyl}-2-methylpropyl]hexanamide (53.3 mg, 0.054 mmol) in DMF (2 ml) was added. The reaction mixture was allowed to warm up to room temperature and was stirred for 16 hours. Solvent was removed under reduced pressure and obtained crude material was purified by reverse phase HPLC to give LP39 (6.5 mg).

LC-MS (ESI): 1214.93[M+H]⁺.

### 39. Synthesis of LP40

The synthesis of LP40 is shown below:

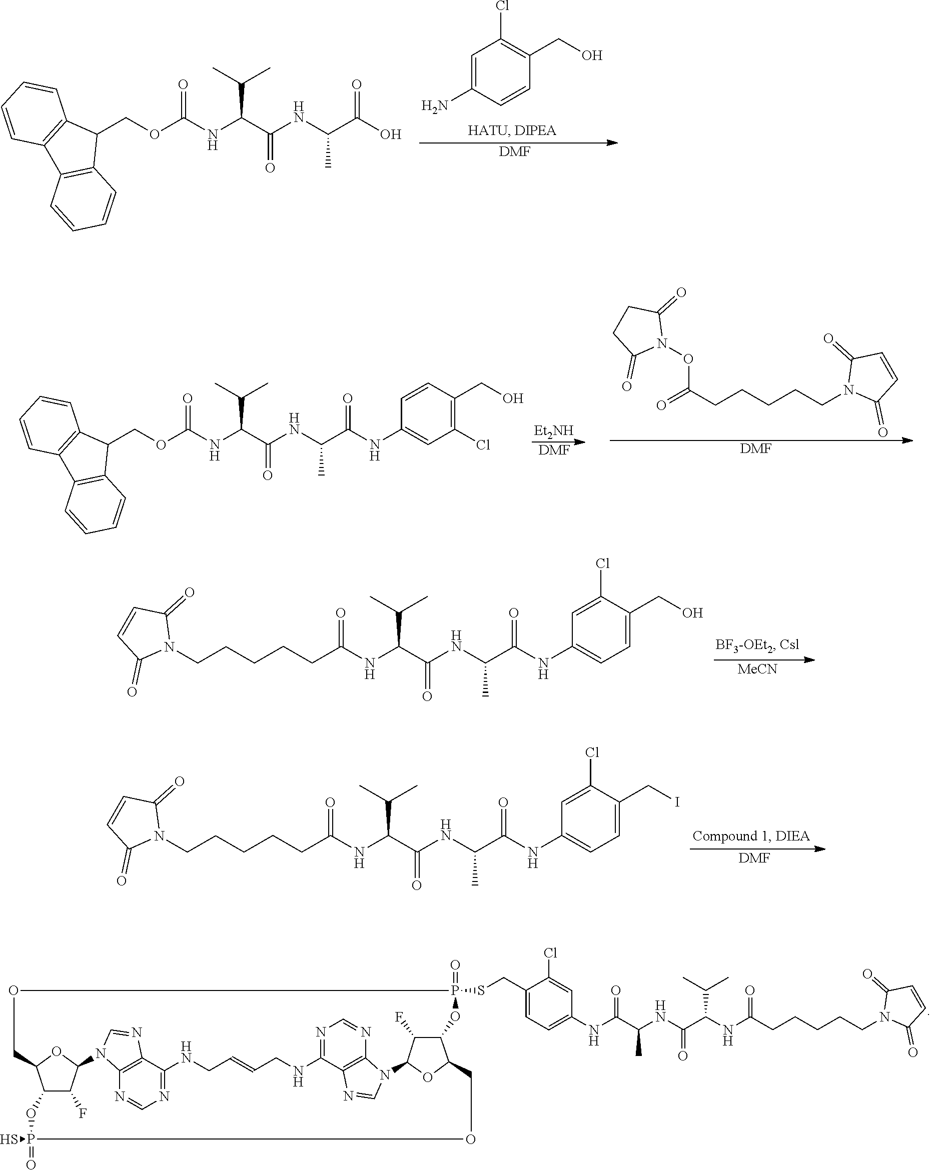

### Synthesis of (9H-fluoren-9-yl)methyl N-[(1S)-1-{[(1S)-1-{[3-chloro-4-(hydroxymethyl)phenyl]carbamoyl}ethyl]carbamoyl}-2-methylpropyl]carbamate

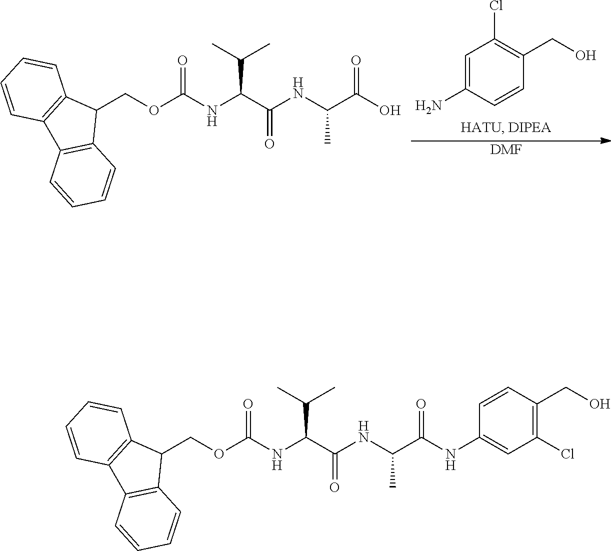

To a solution of (25)-2-[(25)-2-({[(9H-fluoren-9-yl)methoxy]carbonyl}amino)-3-methylbutanamido]propanoic acid (600 mg, 1.462 mmol) and (4-amino-2-chlorophenyl)methanol (346 mg, 2.193 mmol) in DMF were added DIPEA (1277 μl, 7.309 mmol) and then HATU (1112 mg, 2.923 mmol). The reaction was stirred at room temperature for 2 hours, then DMF was removed under reduced pressure. Water (50 mL) was added to the residue and appeared precipitate was collected. Precipitate was purified by silica gel chromatography (Hexane/EtOAc=50/50 to 0/100) to give a product (197 mg). ¹H NMR (400 MHz, METHANOL-d4) δ ppm 0.87-1.11 (m, 6H) 1.11-1.35 (m, 3H) 1.45 (dd, J=7.15, 1.88 Hz, 2H) 1.95-2.14 (m, 1H) 3.10-3.31 (m, 1H) 3.34-3.38 (m, 1H) 3.80 (br d, J=8.53 Hz, 1H) 3.96 (br d, J=6.90 Hz, 1H) 4.07-4.31 (m, 1H) 4.34-4.57 (m, 3H) 4.61 (s, 1H) 4.66 (s, 1H) 7.25-7.50 (m, 5H) 7.53-7.73 (m, 3H) 7.76-7.82 (m, 2H).

### Synthesis of N-[(1S)-1-{[(1S)-1-{[3-chloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide

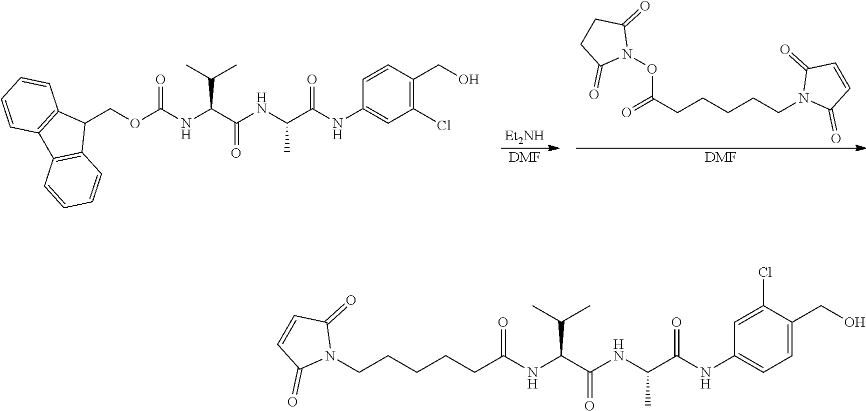

To a solution of (9H-fluoren-9-yl)methyl ((S)-1-(((S)-1-((3-chloro-4-(hydroxymethyl)phenyl)amino)-1-oxopropan-2-yl)amino)-3-methyl-1-oxobutan-2-yl)carbamate (196 mg, 0.357 mmol) in DMF (5 ml) was added diethylamine (0.169 ml, 1.622 mmol). The reaction mixture was stirred at room temperature for 1 hour and then solvent was removed under reduced pressure. Obtained residue was dissolved in DMF (5 ml) and was added 2,5-dioxopyrrolidin-1-yl 6-(2,5-dioxo-2,5-dihydro-1H-pyrrol-1-yl)hexanoate (100 mg, 0.324 mmol) was added and stirred for 2 hours at room temperature. Solvent was removed under reduced pressure, and obtained residue was purified by reverse phase silica gel chromatography (H₂O/MeCN/HCOOH=95/5/0.1 to 50/50/0.1) to give a crude product. The crude product was suspended in DCM/MeOH (9/1), and the precipitate was collected and dried to give a product (97.0 mg) as a white solid. ¹H NMR (400 MHz, METHANOL-d4) δ ppm 0.92-1.14 (m, 6H) 1.14-1.36 (m, 4H) 1.42-1.69 (m, 7H) 2.07 (br d, J=16.06 Hz, 1H) 2.22-2.34 (m, 2H) 2.83-2.89 (m, 1H) 3.01 (s, 1H) 3.34-3.53 (m, 2H) 3.96 (br d, J=8.41 Hz, 1H) 4.18 (br s, 1H) 4.43-4.58 (m, 1H) 4.66 (s, 2H) 6.78 (s, 1H) 6.79-6.82 (m, 1H) 7.40-7.51 (m, 1H) 7.64-7.71 (m, 1H) 7.79 (s, 1H) 7.87 (s, 1H) 8.00 (s, 1H).

### Synthesis of N-[(1S)-1-{[(1S)-1-{[3-chloro-4-(iodomethyl)phenyl]carbamoyl}ethyl]carbamoyl}-2-methylpropyl]-6-(2,5-dioxo-2,5-dihydro-1H-pyrrol-1-yl)hexanamide

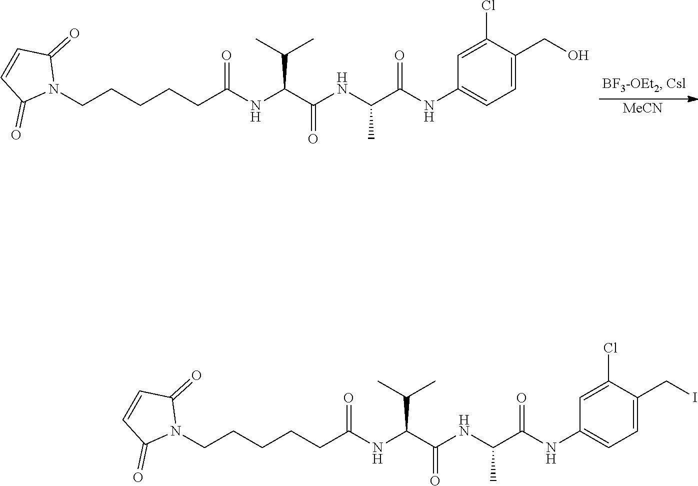 [SMILES: "MeCN" ; CC(C)[C@H](NC(=O)CCCCCN1C(=O)C=CC1=O)C(=O)N[C@@H](C)C(=O)Nc1ccc(CO)c(Cl)c1 ; CC(C)[C@H](NC(=O)CCCCCN1C(=O)C=CC1=O)C(=O)N[C@@H](C)C(=O)Nc1ccc(CI)c(Cl)c1]

To a solution of N-((S)-1-(((S)-1-((3-chloro-4-(hydroxymethyl)phenyl)amino)-1-oxopropan-2-yl)amino)-3-methyl-1-oxobutan-2-yl)-6-(2,5-dioxo-2,5-dihydro-1H-pyrrol-1-yl)hexanamide (93.4 mg, 0.179 mmol) in acetonitrile (2 ml) was added Borontrifluoride ether complex (0.029 ml, 0.233 mmol) and then Cesium iodide (55.9 mg, 0.215 mmol) at room temperature. The mixture was stirred at room temperature for 16 hours, and then was diluted with DCM (50 mL). The organic layer was washed with H₂O (10 mL) and sat. NaHCO₃ aq. (10 mL). Combined aqueous layers were extracted with DCM (15 ml×2), and combined organic layers were washed with 5% NaHSO₃ aq. and was dried over Na₂SO₄. Solid was removed with filter and solvent was removed under reduced pressure. Obtained crude material was used in the next step without further purification (80.4 mg, 58% purity by UV on UPLC). LC-MS (ESI): 631.67 [M+H]⁺.

### Synthesis of N-[(1S)-1-{[(1S)-1-{[3-chloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP40)

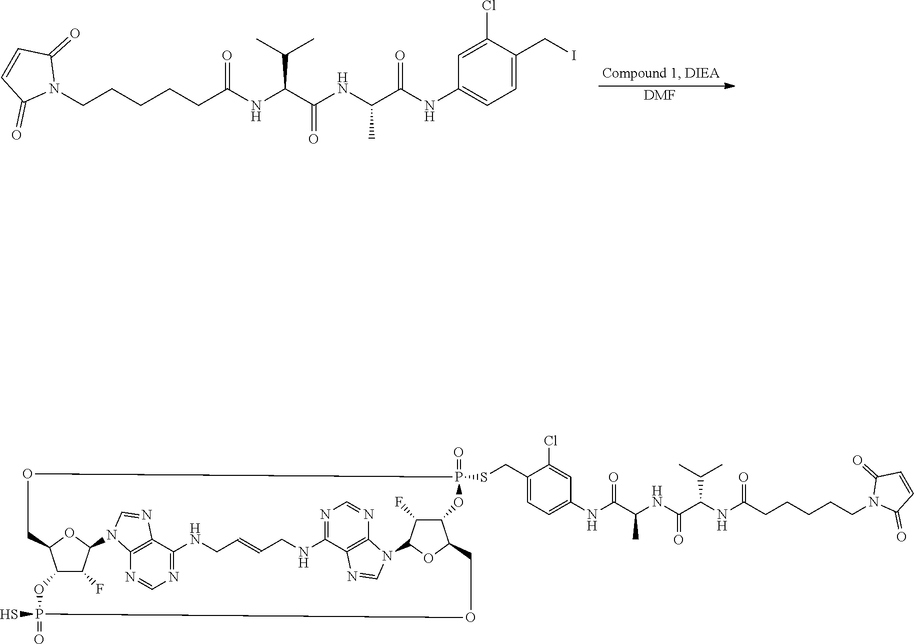

To a suspension of Compound 1 (70 mg, 0.094 mmol) and DIPEA (0.047 ml, 0.268 mmol) in DMF (2 ml) at 0° C., N-[(1S)-1-{[(1S)-1-{[3-chloro-4-(iodomethyl)phenyl]carbamoyl}ethyl]carbamoyl}-2-methylpropyl]-6-(2,5-dioxo-2,5-dihydro-1H-pyrrol-1-yl)hexanamide (79.0 mg, 58% purity, 0.0726 mmol) in DMF (2 ml) was added. The reaction mixture was allowed to warm up to room temperature and was stirred for 16 hours. Solvent was removed under reduced pressure and obtained crude material was purified by reverse phase HPLC to give LP40 (9.7 mg).

LC-MS (ESI): 1249.01 [M+H]⁺.

### 40. Synthesis of LP41

The synthesis of LP41 is shown below:

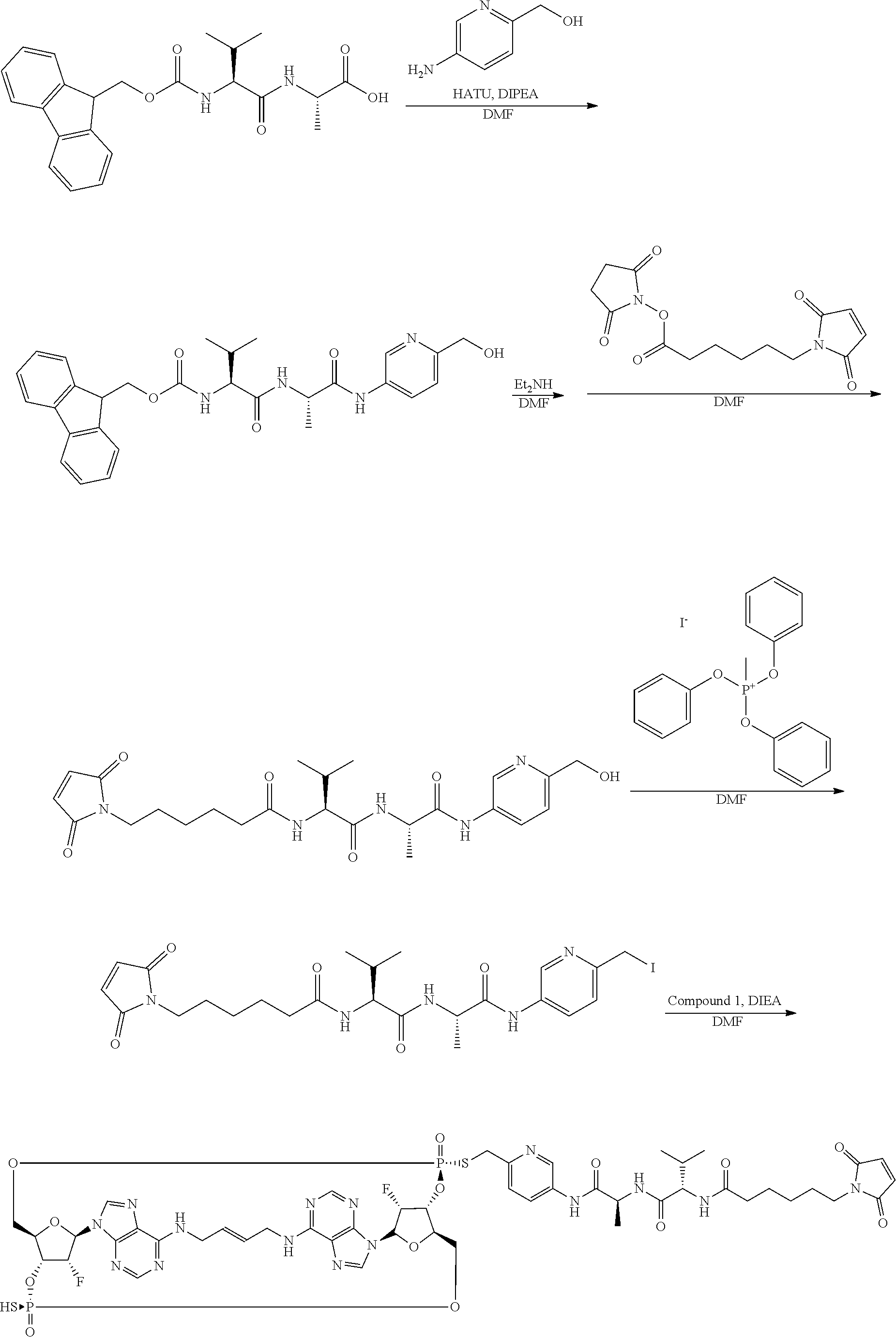

### Synthesis of (9H-fluoren-9-yl)methyl N-[(1S)-1-{[(1S)-1-{[6-(hydroxymethyl)pyridin-3-yl]carbamoyl}ethyl]carbamoyl}-2-methylpropyl]carbamate

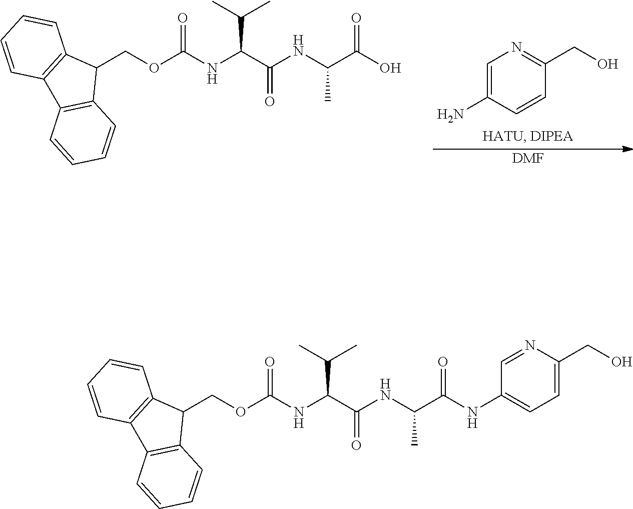

To a solution of (2S)-2-[(2S)-2-({[(9H-fluoren-9-yl)methoxy]carbonyl}amino)-3-methylbutanamido]propanoic acid (840 mg, 2.046 mmol) and (5-aminopyridin-2-yl)methanol (381 mg, 3.07 mmol) in DMF (15 ml) were added DIPEA (1.787 ml, 10.232 mmol) and then HATU (1556 mg, 4.093 mmol) portionwise. The reaction was stirred at room temperature for 2 hours and DMF was removed under reduced pressure. To the obtained residue was added water (50 mL) and appeared precipitate was collected. Precipitate was washed with H₂O and MTBE to give a product as a light brown solid (1.15 g).

### Synthesis of 6-(2,5-dioxo-2,5-dihydro-1H-pyrrol-1-yl)-N-[(1S)-1-{[(1S)-1-{[6-(hydroxymethyl)pyridin-3-yl]carbamoyl}ethyl]carbamoyl}-2-methylpropyl]hexanamide

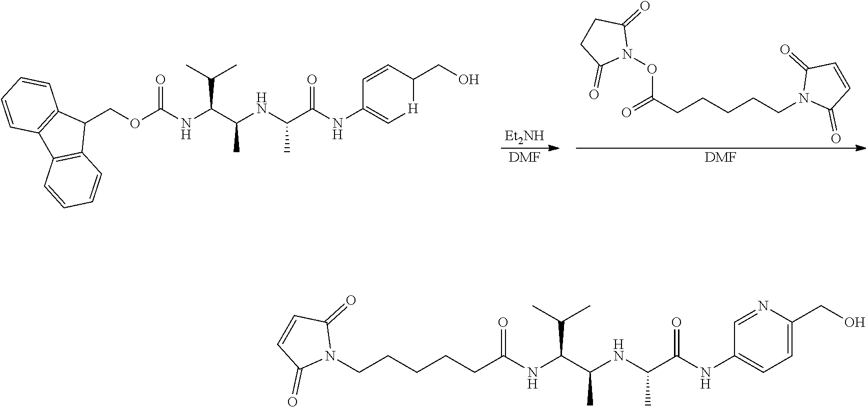

To a solution of (9H-fluoren-9-yl)methyl ((S)-1-(((S)-1-((6-(hydroxymethyl)pyridin-3-yl)amino)-1-oxopropan-2-yl)amino)-3-methyl-1-oxobutan-2-yl)carbamate (1117 mg, 1.946 mmol) in DMF (20 ml) was added diethylamine (0.847 ml, 8.109 mmol). The reaction mixture was stirred at room temperature for 1 hour and then solvent was removed under reduced pressure. Obtained residue was dissolved in DMF (20 ml) and was added 2,5-dioxopyrrolidin-1-yl 6-(2,5-dioxo-2,5-dihydro-1H-pyrrol-1-yl)hexanoate (500 mg, 1.622 mmol) and was stirred at room temperature for 2 hours. Solvent was removed under reduced pressure and the obtained residue was purified by reverse phase chromatography (H₂O/MeCN/HCOOH=95/5/0.1 to 50/50/0.1) to give a product. Product contained impurity, so the material was suspended in DCM/MeOH (9/1), and precipitate was collected and dried to give a white solid (110 mg).

### Synthesis of 6-(2,5-dioxo-2,5-dihydro-1H-pyrrol-1-yl)-N-[(1S)-1-{[(1S)-1-{[6-(iodomethyl)pyridin-3-yl]carbamoyl}ethyl]carbamoyl}-2-methylpropyl]hexanamide

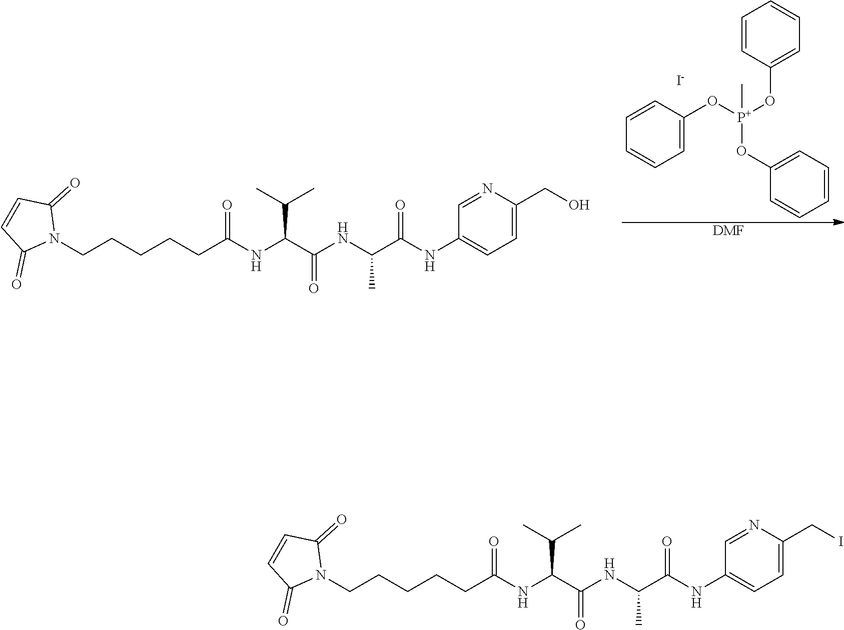

To a solution of 6-(2,5-dioxo-2,5-dihydro-1H-pyrrol-1-yl)-N-((S)-1-(((S)-1-((6-(hydroxymethyl)pyridin-3-yl)amino)-1-oxopropan-2-yl)amino)-3-methyl-1-oxobutan-2-yl)hexanamide (71 mg, 0.146 mmol) in DMF (2 ml) at room temperature was added methyltriphenoxyphosphonium iodide (198 mg, 0.437 mmol) portionwise. The reaction was stirred for 2 hours, and then the mixture was diluted with EtOAc (50 mL) and then the reaction was quenched by addition of 10% NaS₂O₃ aq. (20 mL). Phase separated, and the aqueous layer was extracted with EtOAc (20 mL) once. The combined organic layers were washed with H₂O and Brine (20 mL each), then was dried over Na₂SO₄. Solid was removed with filter and solvent was removed under reduced pressure to give a desired product. The product was used in the next step without further purification (253 mg, 35% purity).

### Synthesis of N-[(1S)-1-{[(1S)-1-{[6-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)pyridin-3-yl]carbamoyl}ethyl]carbamoyl}-2-methylpropyl]-6-(2,5-dioxo-2,5-dihydro-1H-pyrrol-1-yl)hexanamide (LP41)

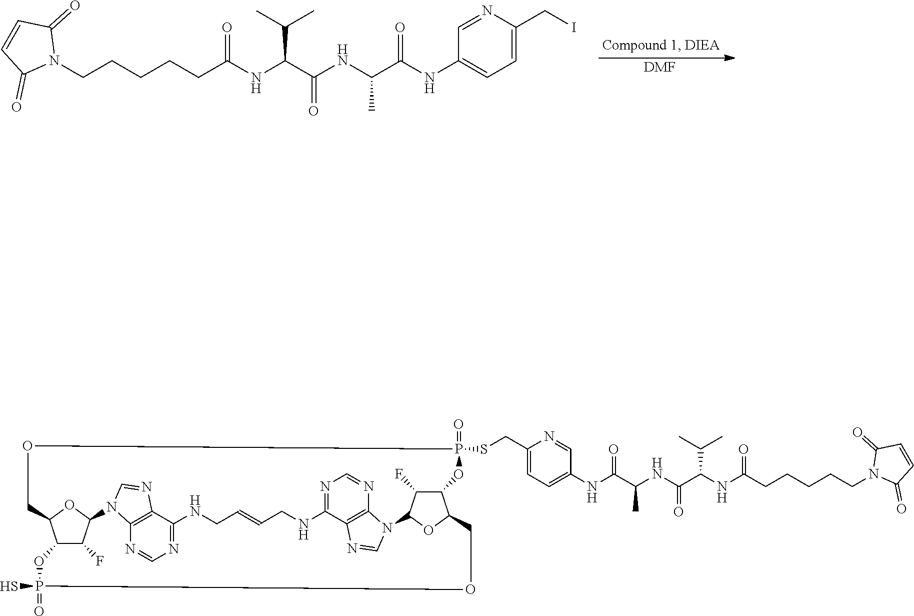

To a solution of Compound 1 (80 mg, 0.107 mmol) and DIPEA (0.094 ml, 0.536 mmol) in DMF (3 ml) at 0° C., 6-(2,5-dioxo-2,5-dihydro-1H-pyrrol-1-yl)-N-[(1S)-1-{[(1S)-1-{[6-(iodomethyl)pyridin-3-yl]carbamoyl}ethyl]carbamoyl}-2-methylpropyl]hexanamide (146 mg, 35% purity, 0.086 mmol) in DMF (3 ml) was added. The reaction mixture was allowed to warm up to room temperature and was stirred for 16 hours. Solvent was removed under reduced pressure, and obtained material was purified by reverse phase HPLC to give LP41 (3.5 mg).

LC-MS (ESI): 1216.14 [M+H]⁺.

### 41. Synthesis of LP42

The synthesis of LP42 is shown below:

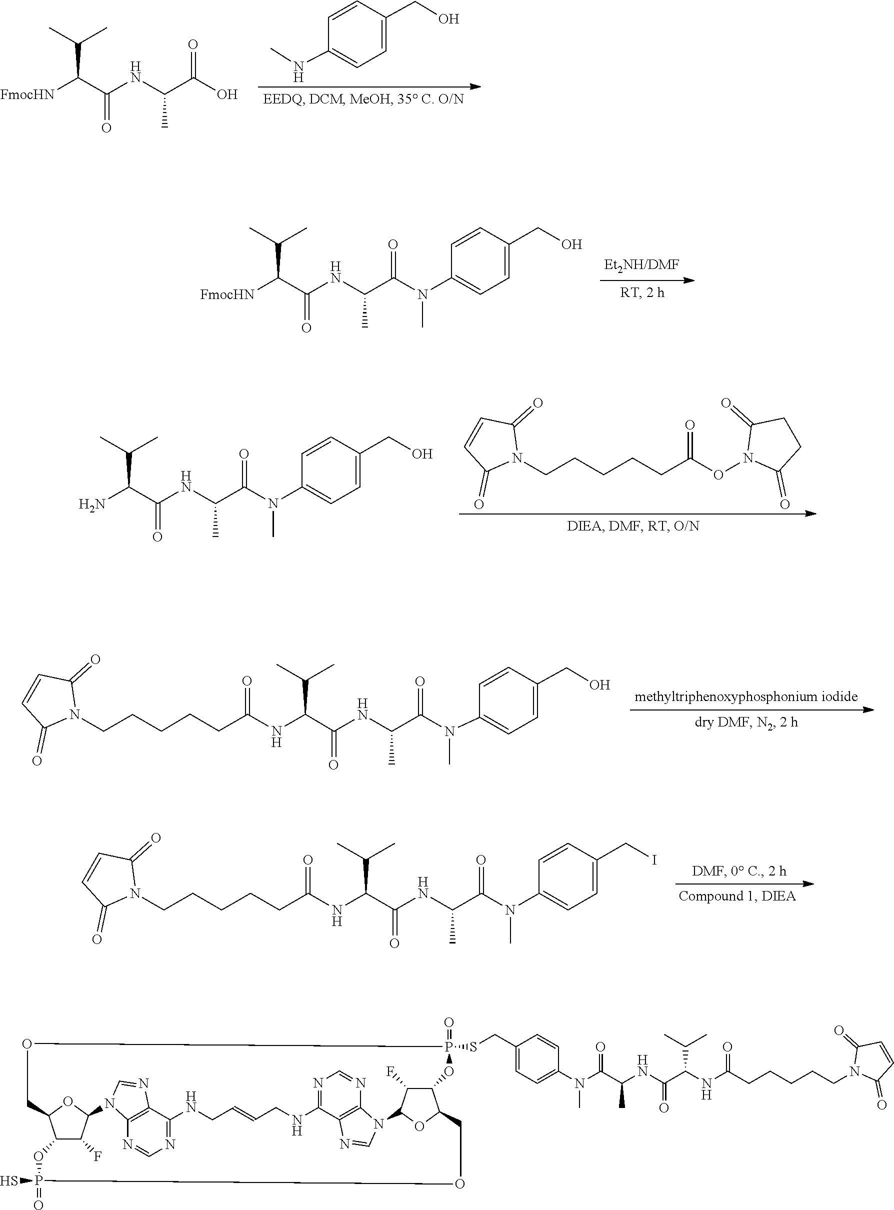

### Synthesis of (9H-fluoren-9-yl)methyl N-[(1S)-1-{[(1S)-1-{[4-(hydroxymethyl)phenyl](methyl)carbamoyl}ethyl]carbamoyl}-2-methylpropyl]carbamate

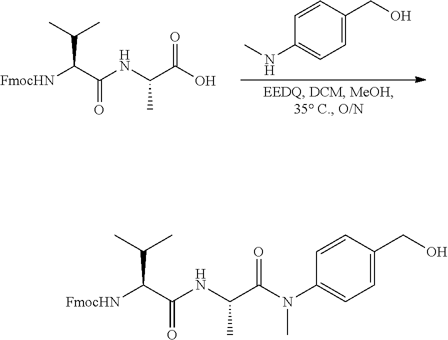

Ethyl 2-ethoxy-1,2-dihydroquinoline-1-carboxylate (1.20 g, 4.872 mmol) was added to a stirred mixture of (2S)-2-[(2S)-2-{[(9H-fluoren-9-ylmethoxy)carbonyl]amino}-3-methylbutanamido]propanoic acid (1 g, 2.436 mmol) and [4-(methylamino)phenyl]methanol (0.33 g, 2.436 mmol) in DCM (18 mL) and MeOH (3 mL) at 25° C. The resulting mixture was stirred at 35° C. for 14 hours. The resulting mixture was concentrated under reduced pressure. The residue was purified by silica gel column chromatography, eluted with PE/EtOAc (1:9) to afford (9H-fluoren-9-yl)methyl N-[(1S)-1-{[(1S)-1-{[4-(hydroxymethyl)phenyl](methyl)carbamoyl}ethyl]carbamoyl}-2-methylpropyl]carbamate (600 mg) as a white solid. LC-MS (ESI): 530.3 [M+H]⁺.

### Synthesis of (2S)-2-amino-N-[(1S)-1-{[4-(hydroxymethyl)phenyl](methyl)carbamoyl}ethyl]-3-methylbutanamide

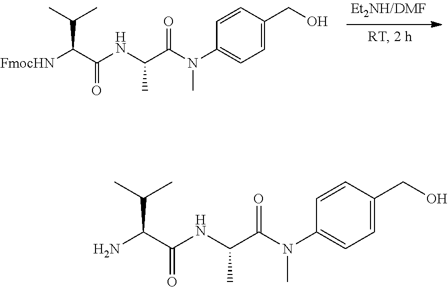

Diethylamine (414.27 mg, 5.665 mmol) was added to a stirred mixture of (9H-fluoren-9-yl)methyl N-[(1S)-1-{[(1S)-1-{[4-(hydroxymethyl)phenyl](methyl)carbamoyl}ethyl]carbamoyl}-2-methylpropyl]carbamate (600 mg, 1.133 mmol) in DMF (5 mL) at 25° C. The resulting mixture was stirred at 25° C. for 2 h. The resulting mixture was concentrated under reduced pressure. The residue was purified by silica gel column chromatography, eluted with CH₂Cl₂/MeOH (4:1) to afford (2S)-2-amino-N-[(1S)-1-{[4-(hydroxymethyl)phenyl](methyl)carbamoyl}ethyl]-3-methylbutanamide (300 mg) as a colorless oil. LC-MS (ESI): 308.2 [M+H]⁺.

### Synthesis of 6-(2,5-dioxo-2,5-dihydro-1H-pyrrol-1-yl)-N-[(1S)-1-{[(1S)-1-{[4-(hydroxymethyl)phenyl](methyl)carbamoyl}ethyl]carbamoyl}-2-methylpropyl]hexanamide

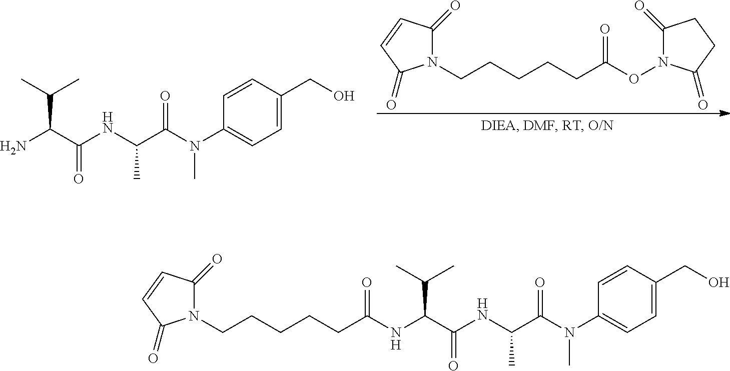 [SMILES: CC(C)[C@H](N)C(=O)N[C@@H](C)C(=O)N(C)c1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)N(C)c1ccc(CO)cc1]

DIEA (252.27 mg, 1.952 mmol) was added to a stirred mixture of (2S)-2-amino-N-[(1S)-1-{[4-(hydroxymethyl)phenyl](methyl)carbamoyl}ethyl]-3-methylbutanamide (300 mg, 0.976 mmol) and 2,5-dioxopyrrolidin-1-yl 6-(2,5-dioxo-2,5-dihydro-1H-pyrrol-1-yl)hexanoate (270.79 mg, 0.878 mmol) in DMF (3 mL) at 25° C. The resulting mixture was stirred at 25° C. for 14 h. The resulting mixture was diluted with EtOAc. The residue was washed with water. The resulting mixture was concentrated under reduced pressure. The residue was purified by silica gel column chromatography, eluted with CH₂Cl₂/MeOH (13:1) to afford 6-(2,5-dioxo-2,5-dihydro-1H-pyrrol-1-yl)-N-[(1S)-1-{[(1S)-1-{[4-(hydroxymethyl)phenyl](methyl)carbamoyl}ethyl]carbamoyl}-2-methylpropyl]hexanamide (400 mg) as a white solid. LC-MS (ESI): 501.3 [M+H]⁺.

### Synthesis of 6-(2,5-dioxo-2,5-dihydro-1H-pyrrol-1-yl)-N-[(1S)-1-{[(1S)-1-{[4-(iodomethyl)phenyl](methyl)carbamoyl}ethyl]carbamoyl}-2-methylpropyl]hexanamide

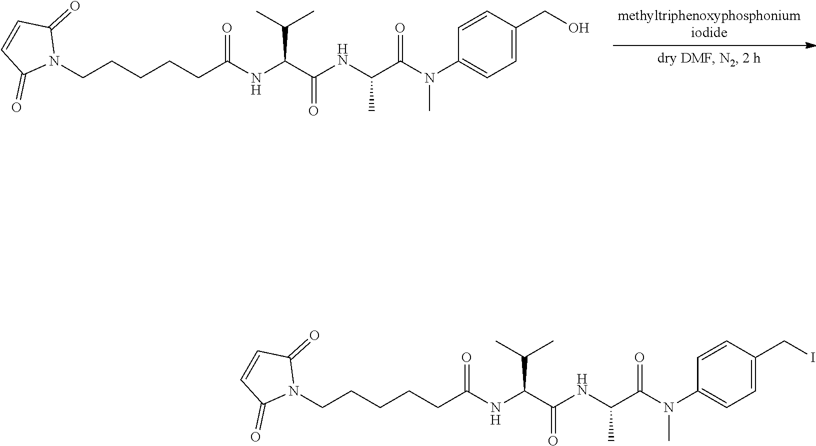 [SMILES: CC(C)[C@H](NC(=O)CCCCCN1C(=O)C=CC1=O)C(=O)N[C@@H](C)C(=O)N(C)c1ccc(CO)cc1 ; CC(C)[C@H](NC(=O)CCCCCN1C(=O)C=CC1=O)C(=O)N[C@@H](C)C(=O)N(C)c1ccc(CI)cc1]

Methyltriphenoxyphosphonium iodide (352.31 mg, 0.780 mmol) was added to a stirred mixture of 6-(2,5-dioxo-2,5-dihydro-1H-pyrrol-1-yl)-N-[(1S)-1-{[(1S)-1-{[4-(hydroxymethyl)phenyl](methyl)carbamoyl}ethyl]carbamoyl}-2-methylpropyl]hexanamide (130 mg, 0.260 mmol) in DMF (3 mL) at 25° C. under nitrogen atmosphere. The resulting mixture was stirred at 25° C. for 5 hours under nitrogen atmosphere. The resulting mixture was diluted with EtOAc. The residue was washed with ice water. The resulting mixture was concentrated under reduced pressure. The residue was purified by Prep-TLC (CH₂Cl₂/IPA 13:1) to afford 6-(2,5-dioxo-2,5-dihydro-1H-pyrrol-1-yl)-N-[(1S)-1-{[(1S)-1-{[4-(iodomethyl)phenyl](methyl)carbamoyl}ethyl]carbamoyl}-2-methylpropyl]hexanamide (110 mg) as a white solid. LC-MS (ESI): 611.2 [M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl](methyl)carbamoyl}ethyl]carbamoyl}-2-methylpropyl]-6-(2,5-dioxo-2,5-dihydro-1H-pyrrol-1-yl)hexanamide (LP42)

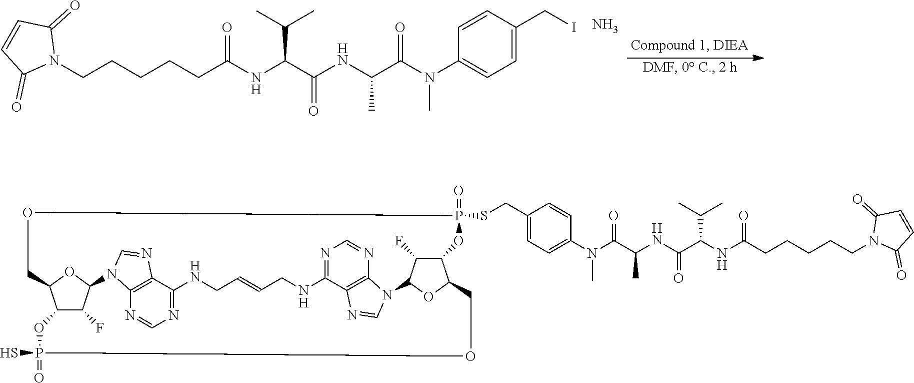

6-(2,5-Dioxo-2,5-dihydro-1H-pyrrol-1-yl)-N-[(1S)-1-{[(1S)-1-{[4-(iodomethyl)phenyl](methyl)carbamoyl}ethyl]carbamoyl}-2-methylpropyl]hexanamide (103.03 mg, 0.169 mmol) was added to a stirred mixture of Compound 1 (70 mg, 0.094 mmol) and DIEA in DMF (3 mL) at 0° C. The resulting mixture was stirred at 0° C. for 2 hours. The crude product was purified by Prep-HPLC with the following conditions (Column: XBridge Prep OBD C18 Column, 19*250 mm, 5 μm; Mobile Phase A: Water(0.1% FA), Mobile Phase B: MeCN; Flow rate: 25 mL/min; Gradient: 20% B to 40% B in 12 min, 40% B to 95% B in 12.2 min, 95% B to 95% B in 14 min, 95% B to 20% B in 14.2 min, 20% B to 20% B in 16 min; Wavelength: 254 nm; RT1(min): 11) to afford N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl](methyl)carbamoyl}ethyl]carbamoyl}-2-methylpropyl]-6-(2,5-dioxo-2,5-dihydro-1H-pyrrol-1-yl)hexanamide (LP42) (13.6 mg) as a white solid.

LC-MS (ESI): 1229.70 [M+H]⁺.

### 42. Synthesis of LP43

The synthesis of L43 is shown below:

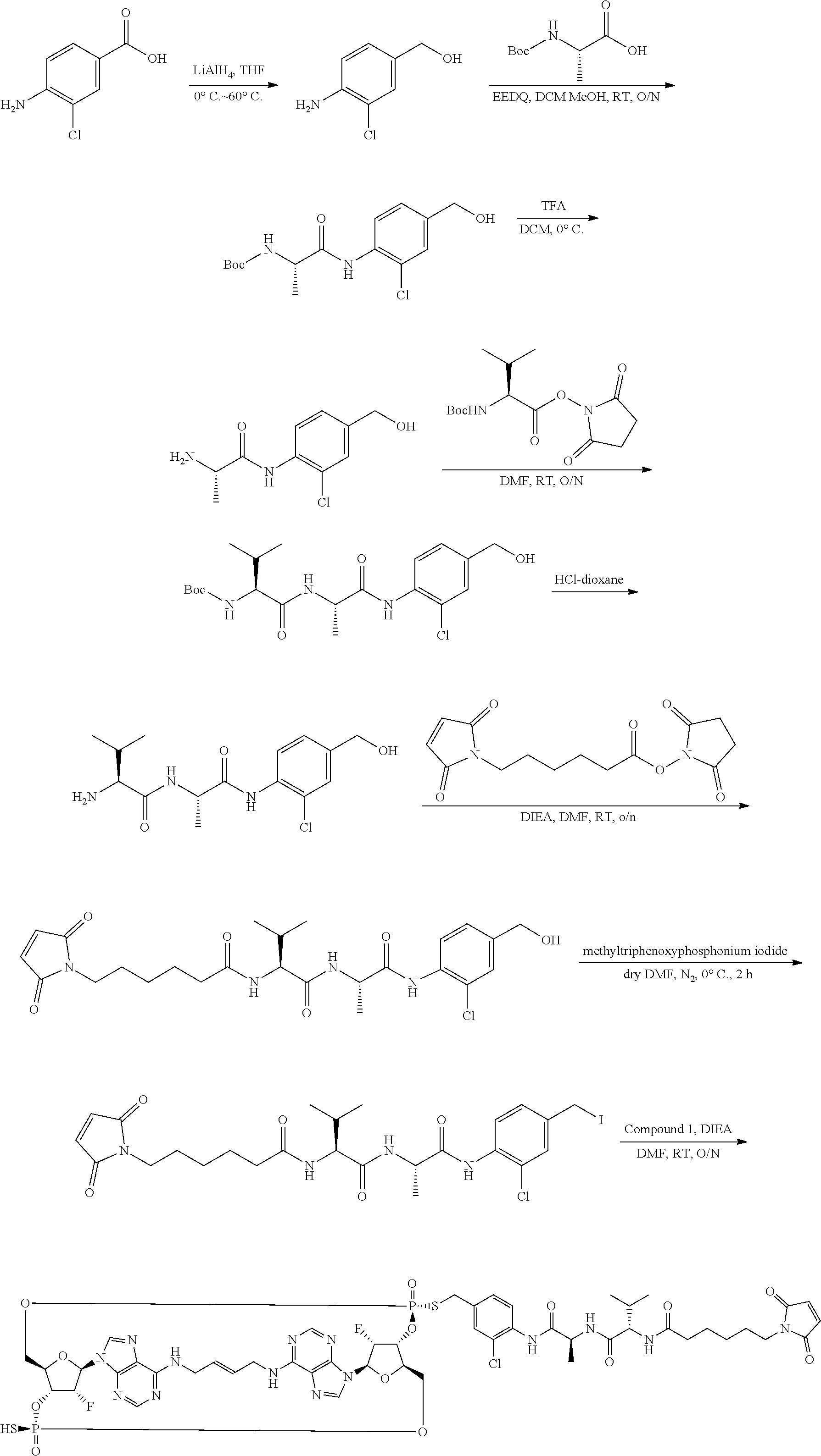

### Synthesis of (4-amino-3-chlorophenyl)methanol

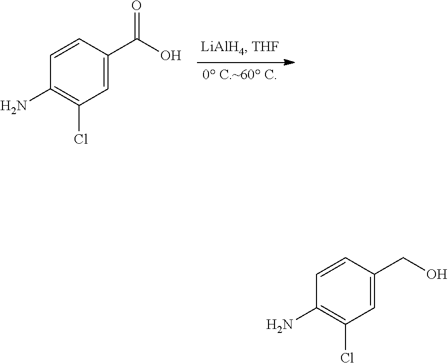 [SMILES: Nc1ccc(C(=O)O)cc1Cl ; Nc1ccc(CO)cc1Cl]

LiAlH₄ (3.32 g, 87.423 mmol) was added to a stirred solution of 4-amino-3-chlorobenzoic acid (5 g, 29.141 mmol) in THF (100 mL) at 0′° C. The resulting mixture was stirred at 60° C. for 14 h under nitrogen atmosphere. The reaction was quenched with MeOH at 0° C. The resulting mixture was concentrated under reduced pressure. The residue was purified by silica gel column chromatography, eluted with PE/EtOAc (3:1) to afford (4-amino-3-chlorophenyl)methanol (1.6 g) as a yellow solid. LC-MS (ESI): 158.03[M+H]⁺.

### Synthesis of tert-butyl N-[(1S)-1-{[2-chloro-4-(hydroxymethyl)phenyl]carbamoyl}ethyl]carbamate

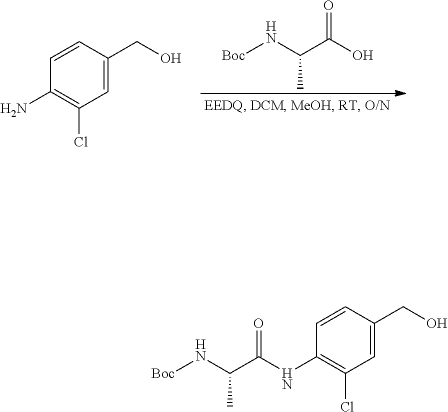 [SMILES: Nc1ccc(CO)cc1Cl ; C[C@H](NC(=O)OC(C)(C)C)C(=O)O ; C[C@H](NC(=O)OC(C)(C)C)C(=O)Nc1ccc(CO)cc1Cl]

EEDQ (7.53 g, 30.456 mmol) and (2S)-2-[(tert-butoxycarbonyl)amino]propanoic acid (2.88 g, 15.228 mmol) were added to a stirred mixture of (4-amino-3-chlorophenyl)methanol (2.4 g, 15.228 mmol) in DCM (12 mL) and MeOH (2 mL). The resulting mixture was stirred at 25° C. for 14 h. The resulting mixture was concentrated under reduced pressure. The residue was purified by silica gel column chromatography, eluted with PE/EtOAc (2:1) to afford tert-butyl N-[(1S)-1-{[2-chloro-4-(hydroxymethyl)phenyl]carbamoyl}ethyl]carbamate (3.7 g) as a white solid. LC-MS (ESI): 329.12[M+H]⁺.

### Synthesis of (2S)-2-amino-N-[2-chloro-4-(hydroxymethyl)phenyl]propanamide

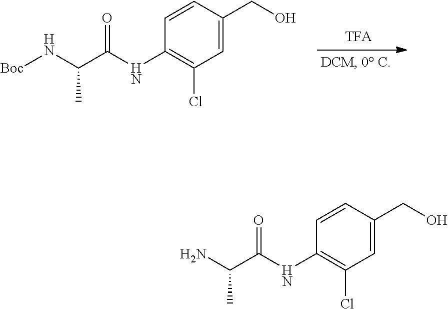 [SMILES: C[C@H](NC(=O)OC(C)(C)C)C(=O)Nc1ccc(CO)cc1Cl ; C[C@H](N)C(=O)Nc1ccc(CO)cc1Cl]

TFA (10.00 mL) was added to a stirred mixture of tert-butyl N-[(1S)-1-{[2-chloro-4-(hydroxymethyl)phenyl]carbamoyl}ethyl]carbamate (3.7 g, 11.253 mmol) in DCM (10 mL) at 0° C. The resulting mixture was stirred at 25° C. for 2 h. The resulting mixture was concentrated under reduced pressure. The residue was purified by silica gel column chromatography, eluted with CH₂Cl₂/MeOH (6:1) to afford (2S)-2-amino-N-[2-chloro-4-(hydroxymethyl)phenyl]propanamide (2.5 g) as a white solid. LC-MS (ESI): 229.07[M+H]⁺.

### Synthesis of tert-butyl N-[(1S)-1-{[(1S)-1-{[2-chloro-4-(hydroxymethyl)phenyl]carbamoyl}ethyl]carbamoyl}-2-methylpropyl]carbamate

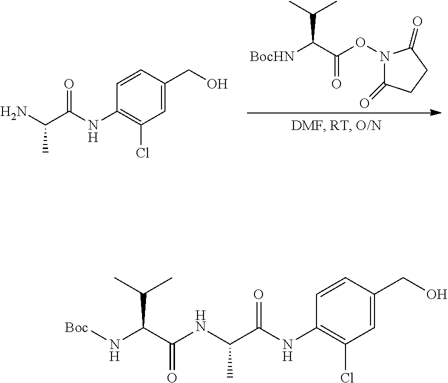 [SMILES: C[C@H](N)C(=O)Nc1ccc(CO)cc1Cl ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1ccc(CO)cc1Cl]

DIEA (2.49 g, 19.240 mmol) and 2,5-dioxopyrrolidin-1-yl (2S)-2-[(tert-butoxycarbonyl)amino]-3-methylbutanoate (3.02 g, 9.620 mmol) were added to a stirred mixture of (2S)-2-amino-N-[2-chloro-4-(hydroxymethyl)phenyl]propanamide (2.2 g, 9.620 mmol) in DMF (6 mL). The resulting mixture was stirred at 25° C. for 14 h. The resulting mixture was concentrated under reduced pressure. The residue was purified by silica gel column chromatography, eluted with PE/EtOAc (1:1) to afford tert-butyl N-[(1S)-1-{[(1S)-1-{[2-chloro-4-(hydroxymethyl)phenyl]carbamoyl}ethyl]carbamoyl}-2-methylpropyl]carbamate (2 g) as a white solid. LC-MS (ESI): 428.19[M+H]⁺.

### Synthesis of (2S)-2-amino-N-[(1S)-1-{[2-chloro-4-(hydroxymethyl)phenyl]carbamoyl}ethyl]-3-methylbutanamide

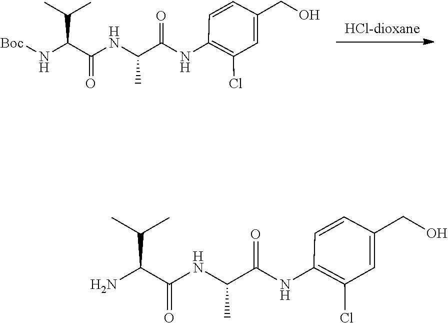 [SMILES: CC(C)[C@H](NC(=O)OC(C)(C)C)C(=O)N[C@@H](C)C(=O)Nc1ccc(CO)cc1Cl ; CC(C)[C@H](N)C(=O)N[C@@H](C)C(=O)Nc1ccc(CO)cc1Cl]

Tert-butyl N-[(1S)-1-{[2-chloro-4-(hydroxymethyl)phenyl]carbamoyl}ethyl]carbamate (2 g, 6.083 mmol) was added to a stirred mixture of HCl(gas) in 1,4-dioxane (10 mL, 329.119 mmol) at 0° C. The resulting mixture was stirred at 25° C. for 2 h. The resulting mixture was concentrated under reduced pressure. The residue was purified by silica gel column chromatography, eluted with CH₂Cl₂/MeOH (3:1) to afford (2S)-2-amino-N-[(1S)-1-{[2-chloro-4-(hydroxymethyl)phenyl]carbamoyl}ethyl]-3-methylbutanamide (1.3 g as a white solid. LC-MS (ESI): 328.14[M+H]⁺.

### Synthesis of N-[(1S)-1-{[(1S)-1-{[2-chloro-4-(hydroxymethyl)phenyl]carbamoyl}ethyl]carbamoyl}-2-methylpropyl]-6-(2,5-dioxopyrrol-1-yl)hexanamide

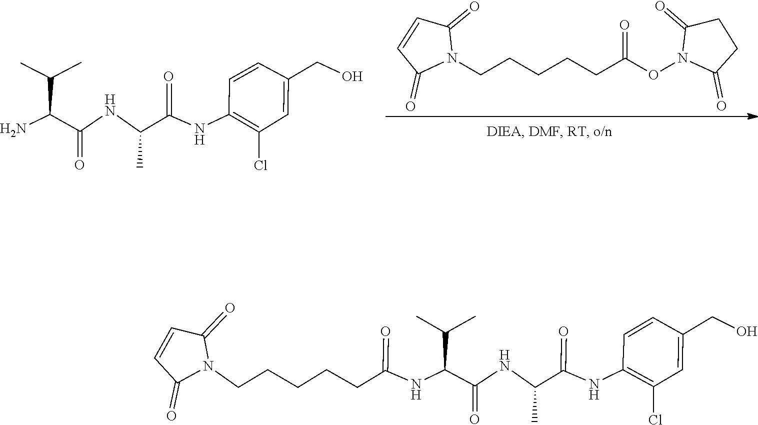 [SMILES: CC(C)[C@H](N)C(=O)N[C@@H](C)C(=O)Nc1ccc(CO)cc1Cl ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1Cl]

DIEA (1.18 g, 9.152 mmol) and 2,5-dioxopyrrolidin-1-yl 6-(2,5-dioxo-2,5-dihydro-1H-pyrrol-1-yl)hexanoate (1.41 g, 4.576 mmol) were added to a stirred mixture of (2S)-2-amino-N-[(1S)-1-{[2-chloro-4-(hydroxymethyl)phenyl]carbamoyl}ethyl]-3-methylbutanamide (1.5 g, 4.576 mmol) in DMF (5 mL). The resulting mixture was stirred at 25° C. for 2 h. The resulting mixture was concentrated under reduced pressure. The residue was purified by reverse flash chromatography with the following conditions: Column: C18; mobile phase A: Water(0.5% FA), mobile phase B: MeCN; gradient: 20% B to 30% B in 30 min; 220/254 nm. This resulted in N-[(1S)-1-{[(1S)-1-{[2-chloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide (1.2 g) as a white solid. LC-MS (ESI): 521.21[M+H]⁺.

### Synthesis of N-[(1S)-1-{[(1S)-1-{[2-chloro-4-(iodomethyl)phenyl]carbamoyl}ethyl]carbamoyl}-2-methylpropyl]-6-(2,5-dioxo-2,5-dihydro-1H-pyrrol-1-yl)hexanamide

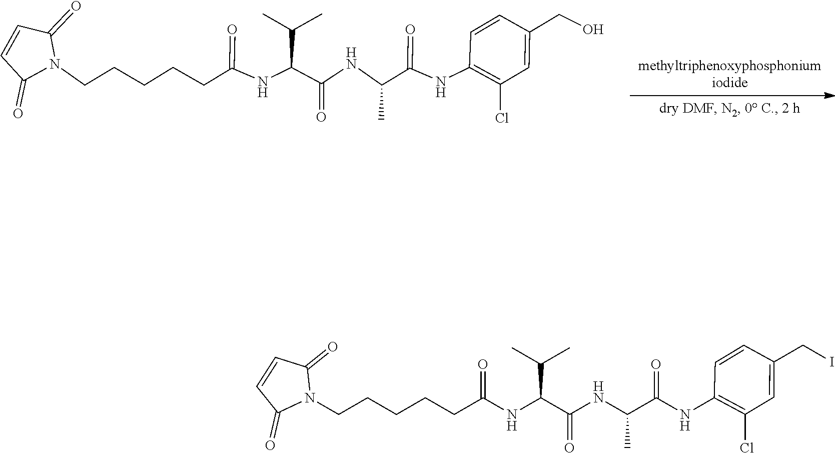 [SMILES: CC(C)[C@H](NC(=O)CCCCCN1C(=O)C=CC1=O)C(=O)N[C@@H](C)C(=O)Nc1ccc(CO)cc1Cl ; CC(C)[C@H](NC(=O)CCCCCN1C(=O)C=CC1=O)C(=O)N[C@@H](C)C(=O)Nc1ccc(CI)cc1Cl]

Methyltriphenoxyphosphonium iodide (520.78 mg, 1.152 mmol) was added to a stirred mixture of N-[(1S)-1-{[(1S)-1-{[2-chloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide (200 mg, 0.384 mmol) in DMF (2 mL). The resulting mixture was stirred at 25° C. for 2 h under nitrogen atmosphere. The resulting mixture was diluted with EtOAc (20 mL). The resulting mixture was washed with 3×10 mL of water. The residue was purified by silica gel column chromatography, eluted with CH₂Cl₂/IPA (10:1) to afford N-[(1S)-1-{[(1S)-1-{[2-chloro-4-(iodomethyl)phenyl]carbamoyl}ethyl]carbamoyl}-2-methylpropyl]-6-(2,5-dioxo-2,5-dihydro-1H-pyrrol-1-yl)hexanamide (140 mg) as a white solid. LC-MS (ESI): 631.11[M+H]⁺.

### Synthesis of N-[(1S)-1-{[(1S)-1-{[2-chloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP43)

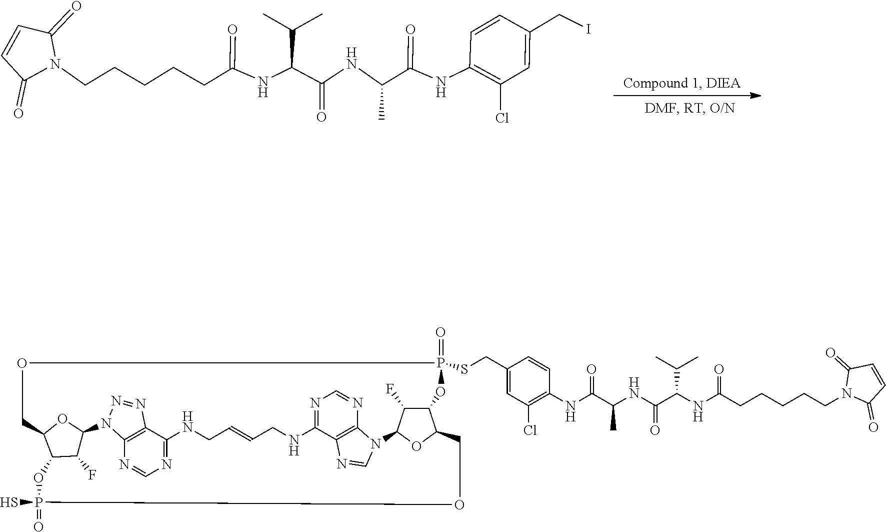 [SMILES: CC(C)[C@H](NC(=O)CCCCCN1C(=O)C=CC1=O)C(=O)N[C@@H](C)C(=O)Nc1ccc(CI)cc1Cl ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)cc1Cl]

N-[(1S)-1-{[(1S)-1-{[2-chloro-4-(iodomethyl)phenyl]carbamoyl}ethyl]carbamoyl}-2-methylpropyl]-6-(2,5-dioxo-2,5-dihydro-1H-pyrrol-1-yl)hexanamide (50.70 mg, 0.080 mmol) and DIEA were added to a stirred mixture of Compound 1 (20 mg, 0.027 mmol) in DMF (1 mL) at 0° C. The resulting mixture was stirred for 3 h at 0° C. The crude product was purified by Prep-HPLC with the following conditions (Column: XBridge Prep OBD C18 Column, 19*250 mm, 5 μm; Mobile Phase A: MeCN, Mobile Phase B: Water(0.1% FA); Flow rate: 25 mL/min; Gradient: 20% B to 40% B in 14 min, 40% B; Wavelength: 254/220 nm; RT1(min): 11.65-12.5; to afford N-[(1S)-1-{[(1S)-1-{[2-chloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP43) (7.8 mg) as a white solid.

LC-MS (ESI): 1249.35[M+H]⁺.

### 43. Synthesis of LP44

The synthesis of LP44 is shown below:

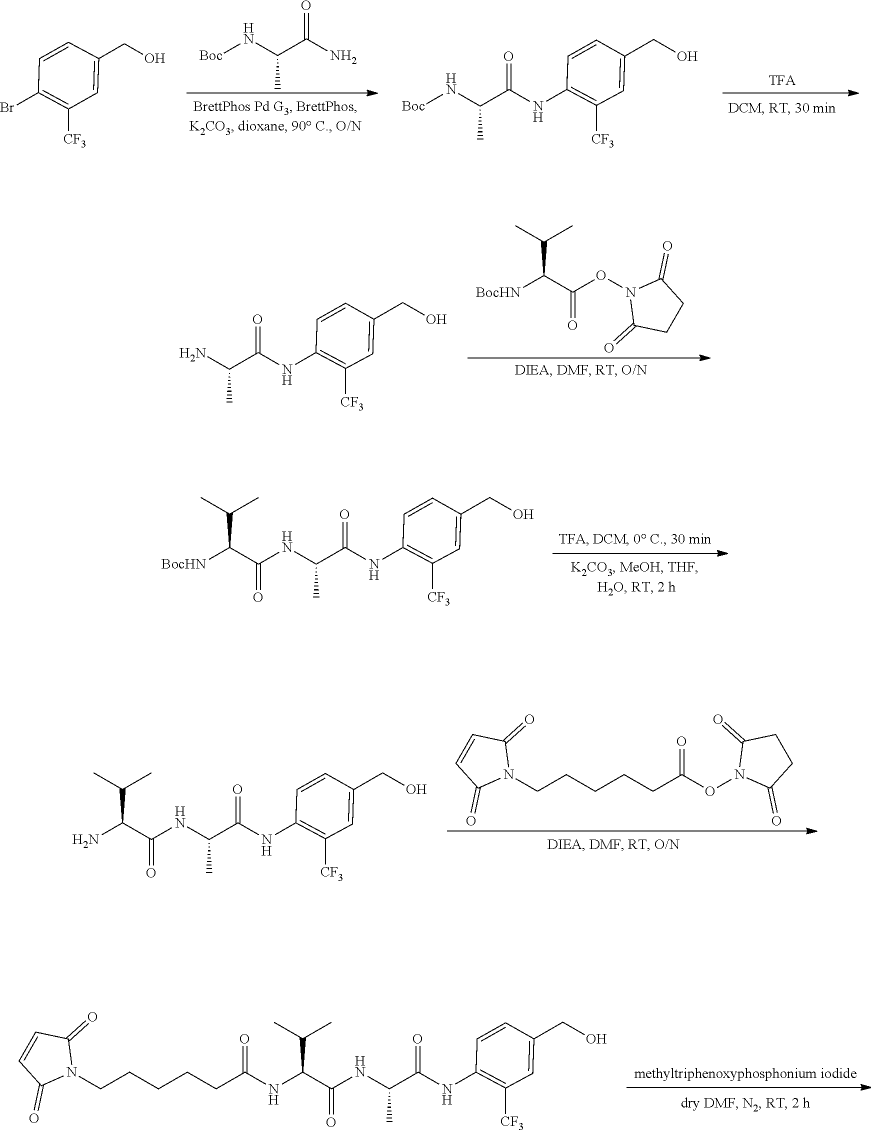

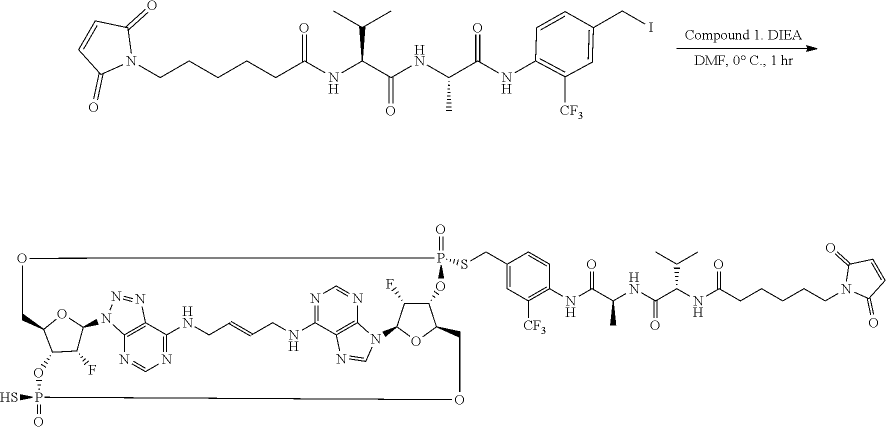 [SMILES: CC(C)[C@H](NC(=O)CCCCCN1C(=O)C=CC1=O)C(=O)N[C@@H](C)C(=O)Nc1ccc(CI)cc1C(F)(F)F ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)cc1C(F)(F)F]

### Synthesis of tert-butyl N-[(1S)-1-{[4-(hydroxymethyl)-2-(trifluoromethyl)phenyl]carbamoyl}ethyl]carbamate

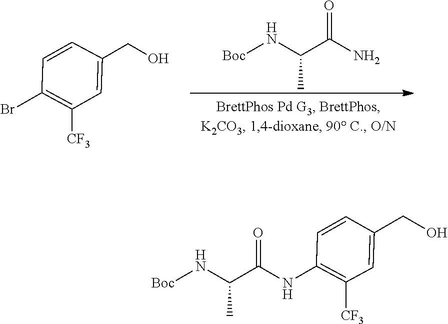 [SMILES: OCc1ccc(Br)c(C(F)(F)F)c1 ; C[C@H](NC(=O)OC(C)(C)C)C(N)=O ; C[C@H](NC(=O)OC(C)(C)C)C(=O)Nc1ccc(CO)cc1C(F)(F)F]

BrettPhos (631.41 mg, 1.176 mmol), BrettPhos Pd G₃ (533.16 mg, 0.588 mmol) and K₂CO₃(1.63 g, 11.763 mmol) were added to a stirred mixture of [4-bromo-3-(trifluoromethyl)phenyl]methanol (1.5 g, 5.882 mmol) and tert-butyl N-[(1S)-1-carbamoylethyl]carbamate (2.21 g, 11.763 mmol) in 1,4-dioxane (20 mL) at 25° C. under nitrogen atmosphere. The resulting mixture was stirred at 90° C. for 14 h under nitrogen atmosphere. The resulting mixture was concentrated under reduced pressure. The resulting mixture was diluted with EtOAc. The resulting mixture was washed with water. The residue was purified by silica gel column chromatography, eluted with PE/EtOAc (3:1) to afford tert-butyl N-[(1S)-1-{[4-(hydroxymethyl)-2-(trifluoromethyl)phenyl]carbamoyl}ethyl]carbamate (930 mg) as a white solid. LC-MS (ESI): 363.2 [M+H]⁺.

### Synthesis of (2S)-2-amino-N-[4-(hydroxymethyl)-2-(trifluoromethyl)phenyl]propanamide

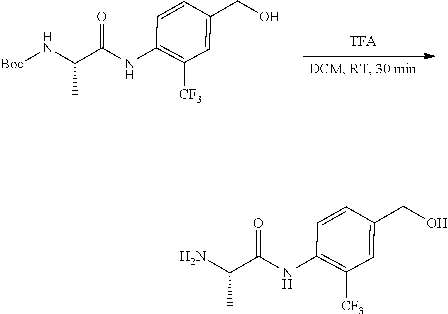 [SMILES: C[C@H](NC(=O)OC(C)(C)C)C(=O)Nc1ccc(CO)cc1C(F)(F)F ; C[C@H](N)C(=O)Nc1ccc(CO)cc1C(F)(F)F]

TFA (6 mL) was added to a stirred mixture of tert-butyl N-[(1S)-1-{[4-(hydroxymethyl)-2-(trifluoromethyl)phenyl]carbamoyl}ethyl]carbamate (900 mg, 2.484 mmol) in DCM (6 mL) at 0° C. The resulting mixture was stirred at 25° C. for 30 min. The resulting mixture was concentrated under reduced pressure. K₂CO₃ (1.37 g, 9.935 mmol) was added to a stirred mixture in MeOH (3 mL), THF (3 mL) and H₂O (3 mL) at 0° C. The resulting mixture was stirred a 25° C. for 2 h. The resulting mixture was concentrated under reduced pressure. The residue was purified by silica gel column chromatography, eluted with CH₂Cl₂/MeOH (5:1) to afford (2S)-2-amino-N-[4-(hydroxymethyl)-2-(trifluoromethyl)phenyl]propanamide (530 mg) as a colorless oil. LC-MS (ESI): 263.1 [M+H]⁺.

### Synthesis of tert-butyl N-[(1S)-1-{[(1S)-1-{[4-(hydroxymethyl)-2-(trifluoromethyl)phenyl]carbamoyl}ethyl]carbamoyl}-2-methylpropyl]carbamate

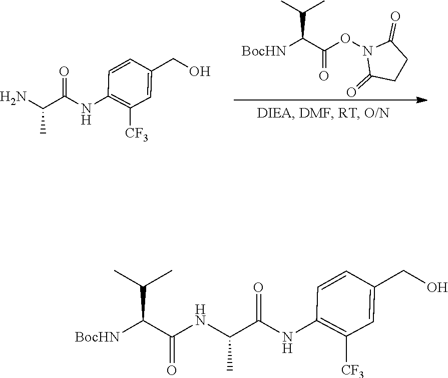 [SMILES: C[C@H](N)C(=O)Nc1ccc(CO)cc1C(F)(F)F ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1ccc(CO)cc1C(F)(F)F]

DIEA (1.48 g, 11.442 mmol) was added to a stirred mixture of (2S)-2-amino-N-[4-(hydroxymethyl)-2-(trifluoromethyl)phenyl]propanamide (500 mg, 1.907 mmol) and 2,5-dioxopyrrolidin-1-yl (2S)-2-[(tert-butoxycarbonyl)amino]-3-methylbutanoate (599.35 mg, 1.907 mmol) in DMF (5 mL) at 25° C. The resulting mixture was stirred at 25° C. for 14 h. The resulting mixture was diluted with EtOAc. The resulting mixture was washed with water. The resulting mixture was concentrated under reduced pressure. The residue was purified by silica gel column chromatography, eluted with PE/EtOAc (1:1) to afford tert-butyl N-[(1S)-1-{[(1S)-1-{[4-(hydroxymethyl)-2-(trifluoromethyl)phenyl]carbamoyl}ethyl]carbamoyl}-2-methylpropyl]carbamate (850 mg) as a white solid. LC-MS (ESI): 462.2 [M+H]⁺.

### Synthesis of (2S)-2-amino-N-[(1S)-1-{[4-(hydroxymethyl)-2-(trifluoromethyl)phenyl]carbamoyl}ethyl]-3-methylbutanamide

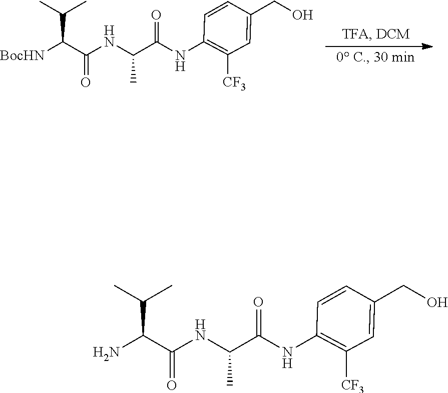 [SMILES: CC(C)[C@H](NC(=O)OC(C)(C)C)C(=O)N[C@@H](C)C(=O)Nc1ccc(CO)cc1C(F)(F)F ; CC(C)[C@H](N)C(=O)N[C@@H](C)C(=O)Nc1ccc(CO)cc1C(F)(F)F]

TFA (4 mL) was added to a stirred mixture of tert-butyl N-[(1S)-1-{[(1S)-1-{[4-(hydroxymethyl)-2-(trifluoromethyl)phenyl]carbamoyl}ethyl]carbamoyl}-2-methylpropyl]carbamate (830 mg, 1.799 mmol) in DCM (4 mL) at 0° C. The resulting mixture was stirred for 30 min at 25° C. The resulting mixture was concentrated under reduced pressure. Then K₂CO₃ (994.28 mg, 7.194 mmol) was added to a stirred mixture in THF (3 mL), MeOH (3 mL) and H₂O (3 mL) at 0° C. The resulting mixture was stirred a 25° C. for 2 h. The resulting mixture was concentrated under reduced pressure. The residue was purified by silica gel column chromatography, eluted with CH₂Cl₂/MeOH (5:1) to afford (2S)-2-amino-N-[(1S)-1-{[4-(hydroxymethyl)-2-(trifluoromethyl)phenyl]carbamoyl}ethyl]-3-methylbutanamide (500 mg) as a white solid. LC-MS (ESI): 362.2 [M+H]⁺.

### Synthesis of 6-(2,5-dioxo-2,5-dihydro-1H-pyrrol-1-yl)-N-[(1S)-1-{[(1S)-1-{[4-(hydroxymethyl)-2-(trifluoromethyl)phenyl]carbamoyl}ethyl]carbamoyl}-2-methylpropyl]hexanamide

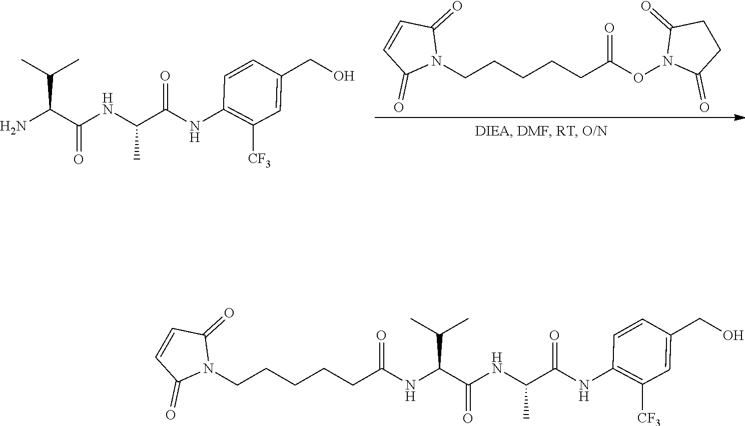 [SMILES: CC(C)[C@H](N)C(=O)N[C@@H](C)C(=O)Nc1ccc(CO)cc1C(F)(F)F ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1C(F)(F)F]

DIEA (357.65 mg, 2.768 mmol) was added to a stirred mixture of (2S)-2-amino-N-[(1S)-1-{[4-(hydroxymethyl)-2-(trifluoromethyl)phenyl]carbamoyl}ethyl]-3-methylbutanamide (500 mg, 1.384 mmol) and 2,5-dioxopyrrolidin-1-yl 6-(2,5-dioxo-2,5-dihydro-1H-pyrrol-1-yl)hexanoate (383.91 mg, 1.246 mmol) in DMF (5 mL) at 25° C. The resulting mixture was stirred at 25° C. for 14 h. The resulting mixture was diluted with EtOAc. The resulting mixture was washed with water. The resulting mixture was concentrated under reduced pressure. The residue was purified by silica gel column chromatography, eluted with CH₂Cl₂/MeOH (11:1) to afford 6-(2,5-dioxo-2,5-dihydro-1H-pyrrol-1-yl)-N-[(1S)-1-{[(1S)-1-{[4-(hydroxymethyl)-2-(trifluoromethyl)phenyl]carbamoyl}ethyl]carbamoyl}-2-methylpropyl]hexanamide (530 mg) as a white solid. LC-MS (ESI): 555.2 [M+H]⁺.

### Synthesis of 6-(2,5-dioxo-2,5-dihydro-1H-pyrrol-1-yl)-N-[(1S)-1-{[(1S)-1-{[4-(iodomethyl)-2-(trifluoromethyl)phenyl]carbamoyl}ethyl]carbamoyl}-2-methylpropyl]hexanamide

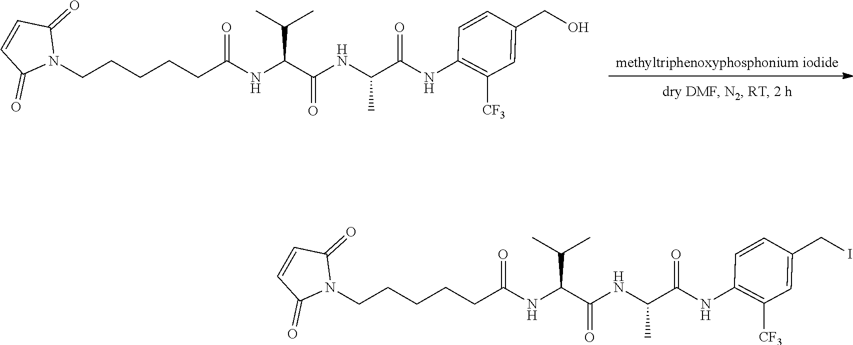 [SMILES: CC(C)[C@H](NC(=O)CCCCCN1C(=O)C=CC1=O)C(=O)N[C@@H](C)C(=O)Nc1ccc(CO)cc1C(F)(F)F ; CC(C)[C@H](NC(=O)CCCCCN1C(=O)C=CC1=O)C(=O)N[C@@H](C)C(=O)Nc1ccc(CI)cc1C(F)(F)F]

Methyltriphenoxyphosphonium iodide (489.76 mg, 1.083 mmol) was added to a stirred mixture of 6-(2,5-dioxo-2,5-dihydro-1H-pyrrol-1-yl)-N-[(1S)-1-{[(1S)-1-{[4-(hydroxymethyl)-2-(trifluoromethyl)phenyl]carbamoyl}ethyl]carbamoyl}-2-methylpropyl]hexanamide (200 mg, 0.361 mmol) in DMF (5 mL) at 0° C. under nitrogen atmosphere. The resulting mixture was stirred at 25° C. for 2 h under nitrogen atmosphere. The resulting mixture was diluted with EtOAc. The residue was washed with ice water. The resulting mixture was concentrated under reduced pressure. The residue was purified by silica gel column chromatography, eluted with CH₂Cl₂/IPA (30:1) to afford 6-(2,5-dioxo-2,5-dihydro-1H-pyrrol-1-yl)-N-[(1S)-1-{[(1S)-1-{[4-(iodomethyl)-2-(trifluoromethyl)phenyl]carbamoyl}ethyl]carbamoyl}-2-methylpropyl]hexanamide (180 mg) as a light yellow solid. LC-MS (ESI): 665.1 [M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-(trifluoromethyl)phenyl]carbamoyl}ethyl]carbamoyl}-2-methylpropyl]-6-(2,5-dioxo-2,5-dihydro-1H-pyrrol-1-yl)hexanamide (LP44)

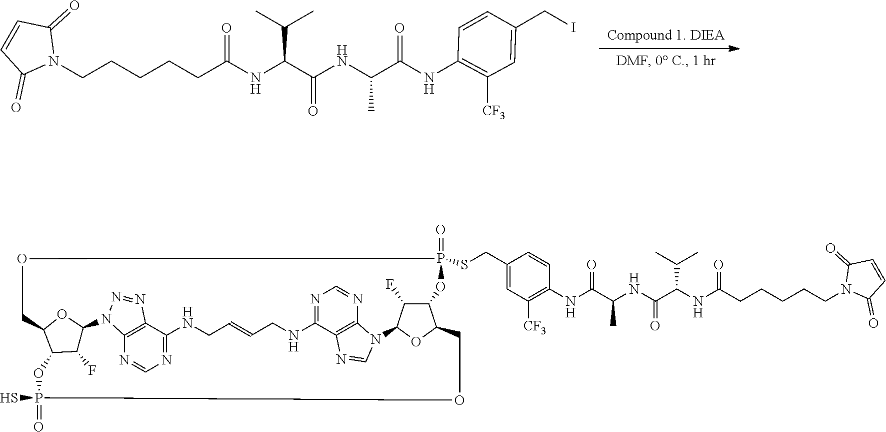 [SMILES: CC(C)[C@H](NC(=O)CCCCCN1C(=O)C=CC1=O)C(=O)N[C@@H](C)C(=O)Nc1ccc(CI)cc1C(F)(F)F ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)cc1C(F)(F)F]

DIEA (57.99 mg, 0.450 mmol) was added to a stirred mixture of 6-(2,5-dioxo-2,5-dihydro-1H-pyrrol-1-yl)-N-[(1S)-1-{[(1S)-1-{[4-(iodomethyl)-2-(trifluoromethyl)phenyl]carbamoyl}ethyl]carbamoyl}-2-methylpropyl]hexanamide (149.07 mg, 0.225 mmol) and Compound 1 (67 mg, 0.090 mmol) in DMF (2 mL) at 0° C. The resulting mixture was stirred at 0° C. for 1 h. The crude product was purified by Prep-HPLC with the following conditions (Column: XBridge Prep OBD C18 Column, 19*250 mm, 5 μm; Mobile Phase A: Water(0.1% FA), Mobile Phase B: MeCN; Flow rate: 25 mL/min; Gradient: 30% B to 30% B in 10 min, 30% B; Wavelength: 254 nm; RT1(min): 8.9) to afford N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-(trifluoromethyl)phenyl]carbamoyl}ethyl]carbamoyl}-2-methylpropyl]-6-(2,5-dioxo-2,5-dihydro-1H-pyrrol-1-yl)hexanamide (LP44) (13.6 mg) as a white solid.

LC-MS (ESI): 1283.40 [M+H]⁺.

### 44. Synthesis of LP45

The synthesis of LP45 is shown below:

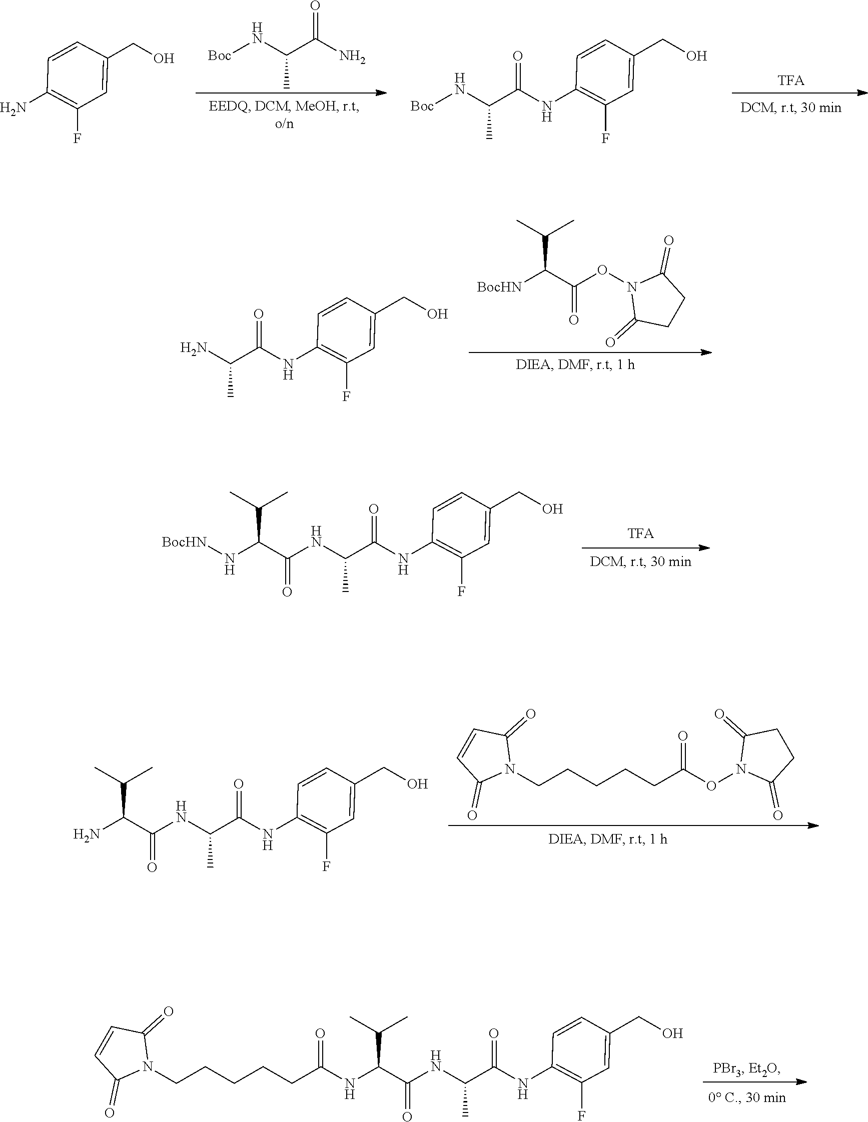

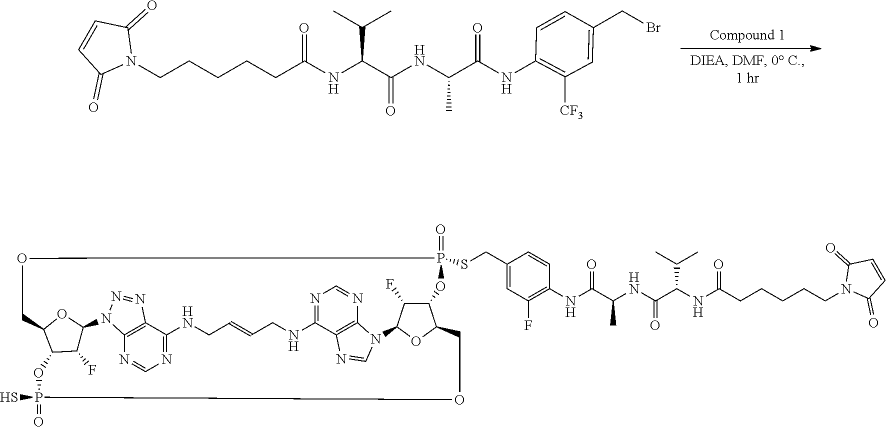 [SMILES: CC(C)[C@H](NC(=O)CCCCCN1C(=O)C=CC1=O)C(=O)N[C@@H](C)C(=O)Nc1ccc(CBr)cc1C(F)(F)F ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)cc1F]

### Synthesis of tert-butyl N-[(1S)-1-{[2-fluoro-4-(hydroxymethyl)phenyl]carbamoyl}ethyl]carbamate

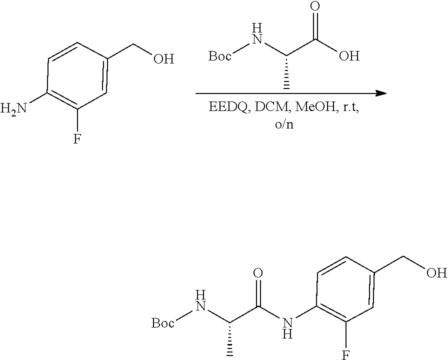 [SMILES: Nc1ccc(CO)cc1F ; C[C@H](NC(=O)OC(C)(C)C)C(=O)O ; C[C@H](NC(=O)OC(C)(C)C)C(=O)Nc1ccc(CO)cc1F]

A mixture of (4-amino-3-fluorophenyl)methanol (750 mg, 5.31 mmol), (2S)-2-[(tert-butoxycarbonyl)amino]propanoic acid (1.21 g, 6.37 mmol) and ethyl 2-ethoxy-1,2-dihydroquinoline-1-carboxylate (2.63 g, 10.62 mmol) in DCM (6 mL) and MeOH (1 mL) was stirred overnight at room temperature. The resulting mixture was concentrated under reduced pressure. The residue was purified by silica gel column chromatography, eluted with PE/EtOAc (2/1) to afford tert-butyl N-[(1S)-1-{[2-fluoro-4-(hydroxymethyl)phenyl]carbamoyl}ethyl]carbamate (1.53 g) as a yellow solid.

### Synthesis of (2S)-2-amino-N-[2-fluoro-4-(hydroxymethyl)phenyl]propanamide

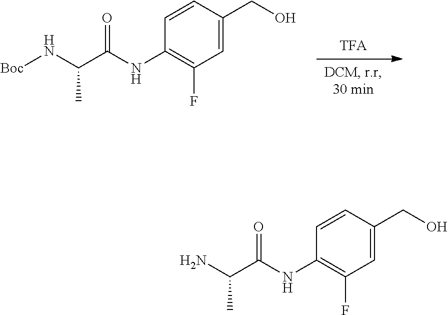 [SMILES: C[C@H](NC(=O)OC(C)(C)C)C(=O)Nc1ccc(CO)cc1F ; C[C@H](N)C(=O)Nc1ccc(CO)cc1F]

A solution of tert-butyl N-[(1S)-1-{[2-fluoro-4-(hydroxymethyl)phenyl]carbamoyl}ethyl]carbamate (700 mg, 2.24 mmol) and TFA (1 mL) in DCM (5 mL) was stirred for 30 min at room temperature. The resulting mixture was concentrated under reduced pressure to afford (2S)-2-amino-N-[2-fluoro-4-(hydroxymethyl)phenyl]propanamide (600 mg, crude) as a yellow oil.

### Synthesis of tert-butyl N-[(1S)-1-{[(1S)-1-{[2-fluoro-4-(hydroxymethyl)phenyl]carbamoyl}ethyl]carbamoyl}-2-methylpropyl]carbamate

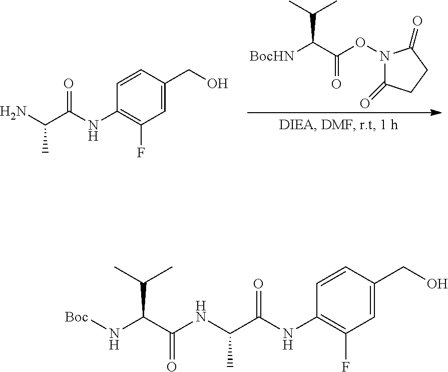 [SMILES: C[C@H](N)C(=O)Nc1ccc(CO)cc1F ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1ccc(CO)cc1F]

A mixture of (2S)-2-amino-N-[2-fluoro-4-(hydroxymethyl)phenyl]propanamide (456 mg, 2.15 mmol) and DIEA (2.78 g, 21.49 mmol) in DMF (10 mL) was stirred for 1 h at room temperature. Reaction was worked up and the residue was purified by silica gel column chromatography, eluted with DCM/EtOAc (3/1) to afford tert-butyl N-[(1S)-1-{[(1S)-1-{[2-fluoro-4-(hydroxymethyl)phenyl]carbamoyl}ethyl]carbamoyl}-2-methylpropyl]carbamate (805 mg) as a yellow solid.

### Synthesis of (2S)-2-amino-N-[(1S)-1-{[2-fluoro-4-(hydroxymethyl)phenyl]carbamoyl}ethyl]-3-methylbutanamide

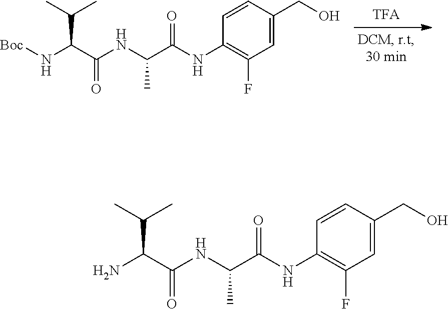 [SMILES: CC(C)[C@H](NC(=O)OC(C)(C)C)C(=O)N[C@@H](C)C(=O)Nc1ccc(CO)cc1F ; CC(C)[C@H](N)C(=O)N[C@@H](C)C(=O)Nc1ccc(CO)cc1F]

A solution of tert-butyl N-[(1S)-1-{[(1S)-1-{[2-fluoro-4-(hydroxymethyl)phenyl]carbamoyl}ethyl]carbamoyl}-2-methylpropyl]carbamate (795 mg, 1.93 mmol) and TFA (2 mL) in DCM (6 mL) was stirred for 30 min at room temperature. The resulting mixture was concentrated under reduced pressure to afford (2S)-2-amino-N-[(1S)-1-{[2-fluoro-4-(hydroxymethyl)phenyl]carbamoyl}ethyl]-3-methylbutanamide (600 mg, crude) as a yellow oil.

### Synthesis of 6-(2,5-dioxo-2,5-dihydro-1H-pyrrol-1-yl)-N-[(1S)-1-{[(1S)-1-{[2-fluoro-4-(hydroxymethyl)phenyl]carbamoyl}ethyl]carbamoyl}-2-methylpropyl]hexanamide

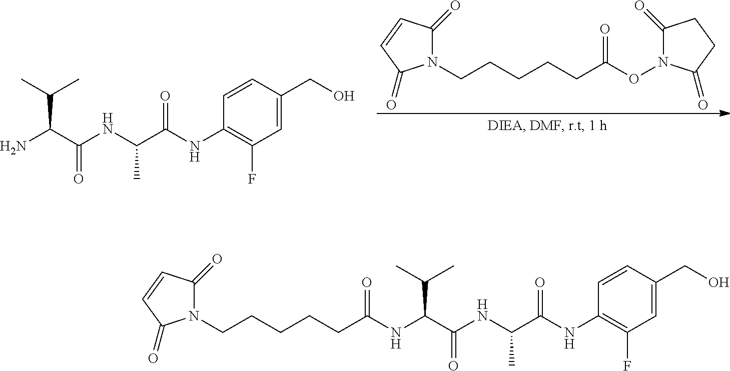 [SMILES: CC(C)[C@H](N)C(=O)N[C@@H](C)C(=O)Nc1ccc(CO)cc1F ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1F]

A mixture of (2S)-2-amino-N-[(1S)-1-{[2-fluoro-4-(hydroxymethyl)phenyl]carbamoyl}ethyl]-3-methylbutanamide (500 mg, 1.60 mmol), 2,5-dioxopyrrolidin-1-yl 6-(2,5-dioxo-2,5-dihydro-1H-pyrrol-1-yl)hexanoate (544.58 mg, 1.76 mmol) and DIEA (2075.48 mg, 16.06 mol) in DMF (5 mL) was stirred for 1 h at room temperature. Reaction was worked up and the residue was purified by silica gel column chromatography, eluted with PE/EtOAc (3/1) to afford 6-(2,5-dioxo-2,5-dihydro-1H-pyrrol-1-yl)-N-[(1S)-1-{[(1S)-1-{[2-fluoro-4-(hydroxymethyl)phenyl]carbamoyl}ethyl]carbamoyl}-2-methylpropyl]hexanamide (340 mg) as a white solid.

### Synthesis of N-[(1S)-1-{[(1S)-1-{[4-(bromomethyl)-2-fluorophenyl]carbamoyl}ethyl]carbamoyl}-2-methylpropyl]-6-(2,5-dioxo-2,5-dihydro-1H-pyrrol-1-yl)hexanamide

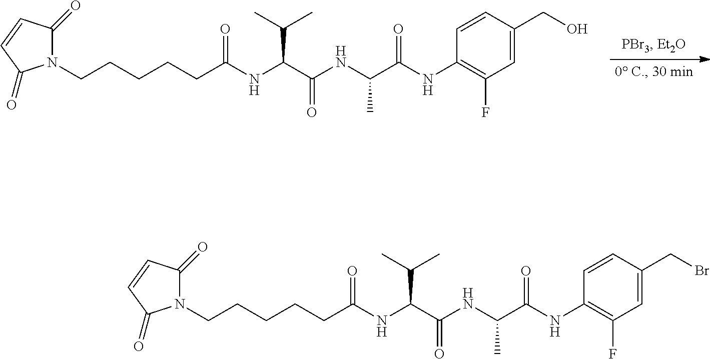 [SMILES: CC(C)[C@H](NC(=O)CCCCCN1C(=O)C=CC1=O)C(=O)N[C@@H](C)C(=O)Nc1ccc(CO)cc1F ; CC(C)[C@H](NC(=O)CCCCCN1C(=O)C=CC1=O)C(=O)N[C@@H](C)C(=O)Nc1ccc(CBr)cc1F]

A solution of 6-(2,5-dioxo-2,5-dihydro-1H-pyrrol-1-yl)-N-[(1S)-1-{[(1S)-1-{[2-fluoro-4-(hydroxymethyl)phenyl]carbamoyl}ethyl]carbamoyl}-2-methylpropyl]hexanamide (150 mg, 0.29 mmol) and PBr₃ (80.47 mg, 0.29 mmol) in Et₂O (2 mL) was stirred for 30 min at 0° C. The resulting mixture was concentrated under reduced pressure. The residue was purified by silica gel column chromatography, eluted with CH₂Cl₂/IPA (10/1) to afford N-[(1S)-1-{[(1S)-1-{[4-(bromomethyl)-2-fluorophenyl]carbamoyl}ethyl]carbamoyl}-2-methylpropyl]-6-(2,5-dioxo-2,5-dihydro-1H-pyrrol-1-yl)hexanamide (90 mg) as a white solid.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-fluorophenyl]carbamoyl}ethyl]carbamoyl}-2-methylpropyl]-6-(2,5-dioxo-2,5-dihydro-1H-pyrrol-1-yl)hexanamide (LP45)

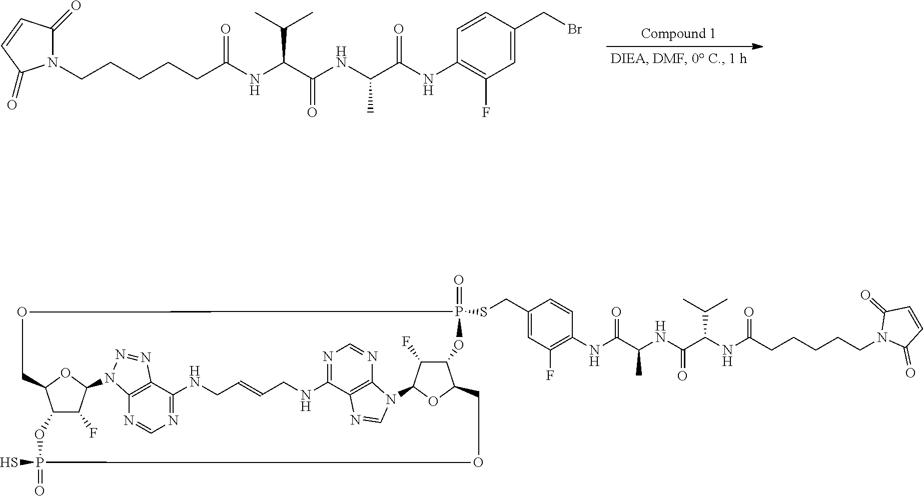 [SMILES: CC(C)[C@H](NC(=O)CCCCCN1C(=O)C=CC1=O)C(=O)N[C@@H](C)C(=O)Nc1ccc(CBr)cc1F ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)cc1F]

A mixture of N-[(1S)-1-{[(1S)-1-{[4-(bromomethyl)-2-fluorophenyl]carbamoyl}ethyl]carbamoyl}-2-methylpropyl]-6-(2,5-dioxo-2,5-dihydro-1H-pyrrol-1-yl)hexanamide (90.81 mg, 0.15 mmol), Compound 1 (58 mg, 0.07 mmol) and DIEA (100.4 mg, 0.78 mmol) in DMF (2 mL) was stirred for 1 h at 0° C. under nitrogen atmosphere. The resulting mixture was concentrated under reduced pressure. The crude product (60 mg) was purified by Prep-HPLC with the following conditions (Column: XBridge Prep Phenyl OBD Column, 19*150 mm, 5 μm; Mobile Phase A: Water(0.1% FA), Mobile Phase B: MeCN; Flow rate: 60 mL/min; Gradient: 27% B to 30% B in 8 min, 30% B; Wavelength: 254 nm) to afford N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-fluorophenyl]carbamoyl}ethyl]carbamoyl}-2-methylpropyl]-6-(2,5-dioxo-2,5-dihydro-1H-pyrrol-1-yl)hexanamide (LP45) (8.5 mg) as a white solid.

LC-MS (ESI): [M−H]⁻: 1231.45.

### 45. Synthesis of LP46

The synthesis of LP46 is shown below:

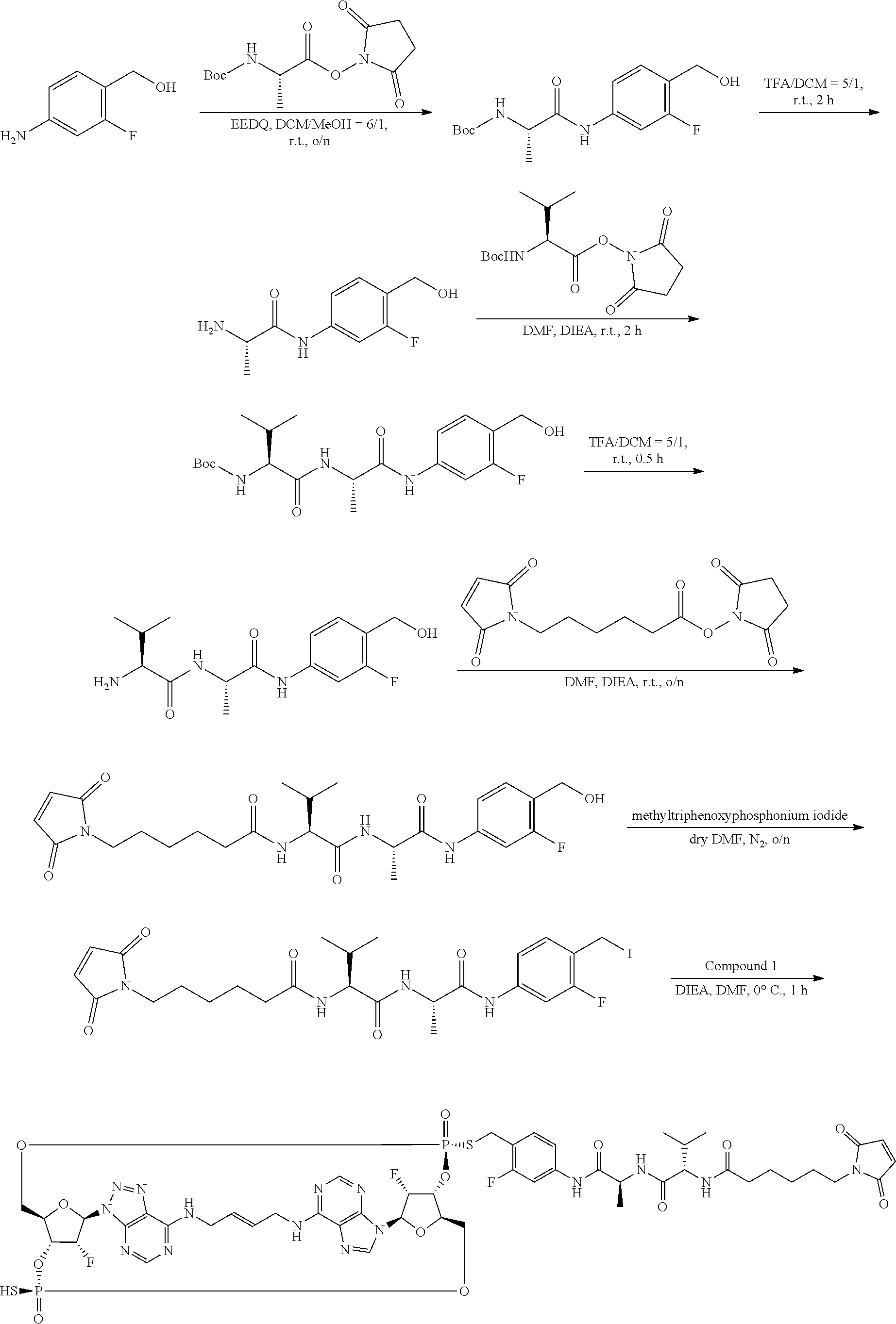

### Synthesis of tert-butyl N-[(1S)-1-{[3-fluoro-4-(hydroxymethyl)phenyl]carbamoyl}ethyl]carbamate

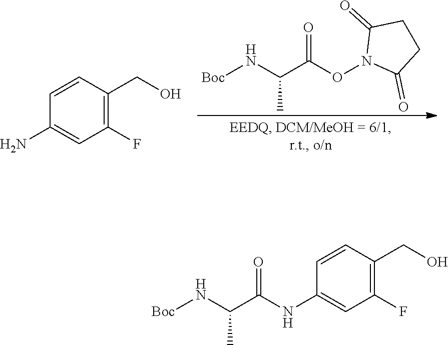 [SMILES: Nc1ccc(CO)c(F)c1 ; C[C@H](NC(=O)OC(C)(C)C)C(=O)ON1C(=O)CCC1=O ; C[C@H](NC(=O)OC(C)(C)C)C(=O)Nc1ccc(CO)c(F)c1]

A solution of (4-amino-2-fluorophenyl)methanol (1 g, 7.145 mmol) and (25)-2-[(tert-butoxycarbonyl)amino]propanoic acid (1.61 g, 8.500 mmol) and ethyl 2-ethoxy-1,2-dihydroquinoline-1-carboxylate (3.54 g, 15.201 mmol) in DCM (12 mL) and MeOH (2 mL) was stirred at room temperature overnight. The resulting mixture was concentrated under vacuum. The residue was purified by silica gel column chromatography, eluted with PE/EtOAc=(1/1) to afford tert-butyl N-[(15)-1-{[3-fluoro-4-(hydroxymethyl)phenyl]carbamoyl}ethyl]carbamate (2.2 g) as a white solid.

### Synthesis of (2S)-2-amino-N-[3-fluoro-4-(hydroxymethyl)phenyl]propanamide

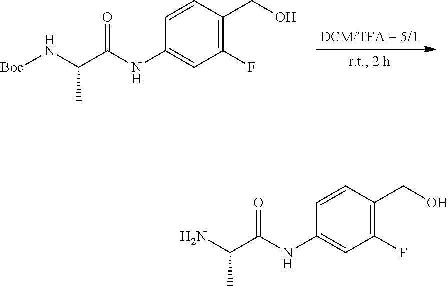 [SMILES: C[C@H](NC(=O)OC(C)(C)C)C(=O)Nc1ccc(CO)c(F)c1 ; C[C@H](N)C(=O)Nc1ccc(CO)c(F)c1]

A solution of tert-butyl N-[(1S)-1-{[3-fluoro-4-(hydroxymethyl)phenyl]carbamoyl}ethyl]carbamate (2 g, 6.404 mmol) in TFA (2 mL) and DCM (10 mL) was stirred for 2 h at room temperature. The resulting mixture was concentrated under vacuum. This resulted in (2S)-2-amino-N-[3-fluoro-4-(hydroxymethyl)phenyl]propanamide (1 g) as a white oil. The resulting mixture was used in the next step directly without further purification.

### Synthesis of tert-butyl N-[(1S)-1-{[(1S)-1-{[3-fluoro-4-(hydroxymethyl)phenyl]carbamoyl}ethyl]carbamoyl}-2-methylpropyl]carbamate

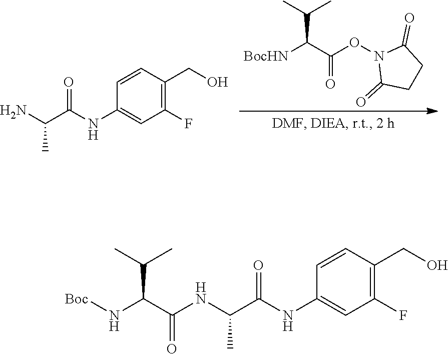 [SMILES: C[C@H](N)C(=O)Nc1ccc(CO)c(F)c1 ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1ccc(CO)c(F)c1]

A solution of (2S)-2-amino-N-[3-fluoro-4-(hydroxymethyl)phenyl]propanamide (1 g, 4.625 mmol) and 2,5-dioxopyrrolidin-1-yl (2S)-2-[(tert-butoxycarbonyl)amino]-3-methylbutanoate (1.7 g, 5.1241 mmol) in DIEA (6.8 g, 49.7152 mmol) and DMF (10 mL) was stirred for 2 h at room temperature. Reaction was worked up and the residue was purified by silica gel column chromatography, eluted with DCM/EtOAc=(1/1) to afford tert-butyl N-[(1S)-1-{[(1S)-1-{[3-fluoro-4-(hydroxymethyl)phenyl]carbamoyl}ethyl]carbamoyl}-2-methylpropyl]carbamate (1.6 g) as a white solid.

### Synthesis of (2S)-2-amino-N-[(1S)-1-{[3-fluoro-4-(hydroxymethyl)phenyl]carbamoyl}ethyl]-3-methylbutanamide

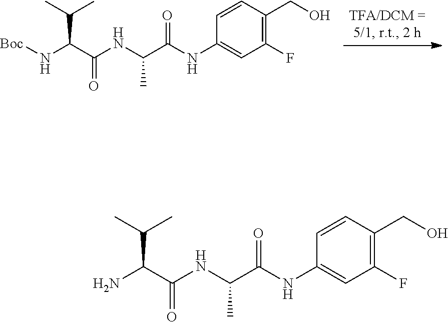 [SMILES: CC(C)[C@H](NC(=O)OC(C)(C)C)C(=O)N[C@@H](C)C(=O)Nc1ccc(CO)c(F)c1 ; CC(C)[C@H](N)C(=O)N[C@@H](C)C(=O)Nc1ccc(CO)c(F)c1]

A solution of tert-butyl N-[(1S)-1-{[(1S)-1-{[3-fluoro-4-(hydroxymethyl)phenyl]carbamoyl}ethyl]carbamoyl}-2-methylpropyl]carbamate (1.6 g, 3.9671 mmol) in TFA (2 mL) and DCM (10 mL) was stirred for 2 h at room temperature. The resulting mixture was concentrated under vacuum. This resulted in (2S)-2-amino-N-[(1S)-1-{[3-fluoro-4-(hydroxymethyl)phenyl]carbamoyl}ethyl]-3-methylbutanamide (1.15 g) as a white oil. The resulting mixture was used in the next step directly without further purification.

### Synthesis of 6-(2,5-dioxo-2,5-dihydro-1H-pyrrol-1-yl)-N-[(1S)-1-{[(1S)-1-{[3-fluoro-4-(hydroxymethyl)phenyl]carbamoyl}ethyl]carbamoyl}-2-methylpropyl]hexanamide

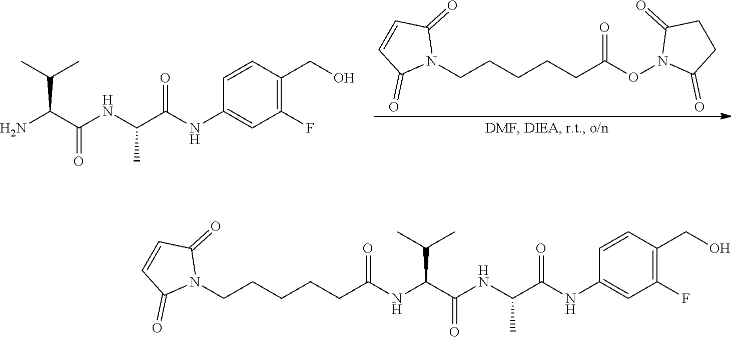 [SMILES: CC(C)[C@H](N)C(=O)N[C@@H](C)C(=O)Nc1ccc(CO)c(F)c1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)c(F)c1]

A solution of (2S)-2-amino-N-[(1S)-1-{[3-fluoro-4-(hydroxymethyl)phenyl]carbamoyl}ethyl]-3-methylbutanamide (1.2 g, 3.5147 mmol) and 2,5-dioxopyrrolidin-1-yl 6-(2,5-dioxo-2,5-dihydro-1H-pyrrol-1-yl)hexanoate (1.2 g, 3.886 mmol) in DIEA (4.6 g, 35.320 mmol) and DMF (10 mL) was stirred at room temperature overnight. Reaction was worked up and the residue was purified by silica gel column chromatography, eluted with DCM/MeOH=(10/1) to afford 6-(2,5-dioxo-2,5-dihydro-1H-pyrrol-1-yl)-N-[(1S)-1-{[(1S)-1-{[3-fluoro-4-(hydroxymethyl)phenyl]carbamoyl}ethyl]carbamoyl}-2-methylpropyl]hexanamide (900 mg) as a white solid.

### Synthesis of 6-(2,5-dioxo-2,5-dihydro-1H-pyrrol-1-yl)-N-[(1S)-1-{[(1S)-1-{[3-fluoro-4-(iodomethyl)phenyl]carbamoyl}ethyl]carbamoyl}-2-methylpropyl]hexanamide

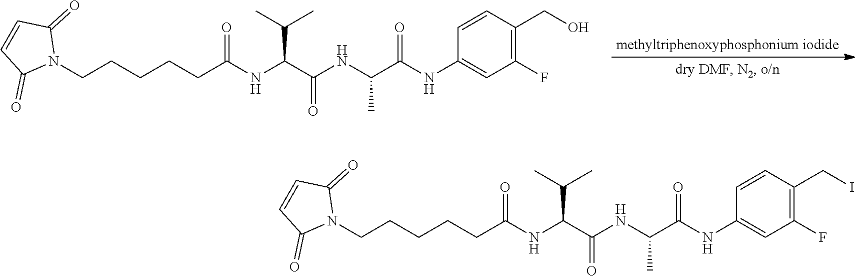 [SMILES: CC(C)[C@H](NC(=O)CCCCCN1C(=O)C=CC1=O)C(=O)N[C@@H](C)C(=O)Nc1ccc(CO)c(F)c1 ; CC(C)[C@H](NC(=O)CCCCCN1C(=O)C=CC1=O)C(=O)N[C@@H](C)C(=O)Nc1ccc(CI)c(F)c1]

A solution of 6-(2,5-dioxo-2,5-dihydro-1H-pyrrol-1-yl)-N-[(1S)-1-{[(1S)-1-{[3-fluoro-4-(hydroxymethyl)phenyl]carbamoyl}ethyl]carbamoyl}-2-methylpropyl]hexanamide (900 mg, 1.798 mmol) and methyltriphenoxyphosphonium iodide (3.7 g, 9.861 mmol) in DMF (9 mL) was stirred at 0° C. under nitrogen atmosphere overnight. The resulting mixture was extracted with EtOAc. The combined organic layers were washed with H₂O, dried over anhydrous Na₂SO₄. After filtration, the filtrate was concentrated under reduced pressure. The residue was purified by silica gel column chromatography, eluted with DCM/IPA=(10/1) to afford 6-(2,5-dioxo-2,5-dihydro-1H-pyrrol-1-yl)-N-[(1S)-1-{[(1S)-1-{[3-fluoro-4-(iodomethyl)phenyl]carbamoyl}ethyl]carbamoyl}-2-methylpropyl]hexanamide (700 mg) as a white solid.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-3-fluorophenyl]carbamoyl}ethyl]carbamoyl}-2-methylpropyl]-6-(2,5-dioxo-2,5-dihydro-1H-pyrrol-1-yl)hexanamide (LP46)

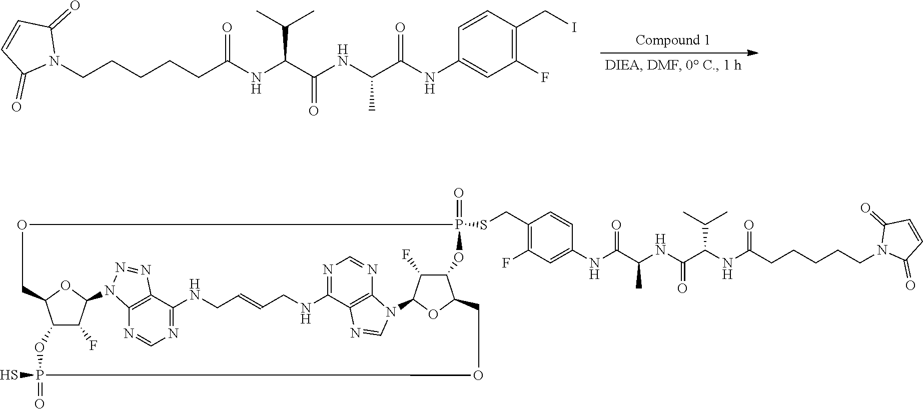 [SMILES: CC(C)[C@H](NC(=O)CCCCCN1C(=O)C=CC1=O)C(=O)N[C@@H](C)C(=O)Nc1ccc(CI)c(F)c1 ; CC(C)[C@H](NC(=O)CCCCCN1C(=O)C=CC1=O)C(=O)N[C@@H](C)C(=O)Nc1ccc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2nnn34)c(F)c1]

A solution of Compound 1 (55 mg, 0.082 mmol) and 6-(2,5-dioxo-2,5-dihydro-1H-pyrrol-1-yl)-N-[(1S)-1-{[(1S)-1-{[3-fluoro-4-(iodomethyl)phenyl]carbamoyl}ethyl]carbamoyl}-2-methylpropyl]hexanamide (95.3 mg, 0.1530 mmol) in DIEA (51.93 mg, 0.390 mmol) and DMF (1 mL) was stirred for 1 h at 0° C. under nitrogen atmosphere. The crude product was purified by reverse phase flash with the following conditions (Column: Xselect CSH C18 OBD Column 30×150 mm 5 μm; Mobile Phase A: Water(0.1% FA), Mobile Phase B: MeCN; Flow rate: 60 mL/min; Gradient: 26% B to 30% B in 8 min, 30% B; Wavelength: 254 nm to afford N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-3-fluorophenyl]carbamoyl}ethyl]carbamoyl}-2-methylpropyl]-6-(2,5-dioxo-2,5-dihydro-1H-pyrrol-1-yl)hexanamide (LP46) (3.3 mg) as a white solid.

LC-MS (ESI): [M+H]⁺: 1233.2.

### 46. Synthesis of LP47

The synthesis of LP47 is shown below:

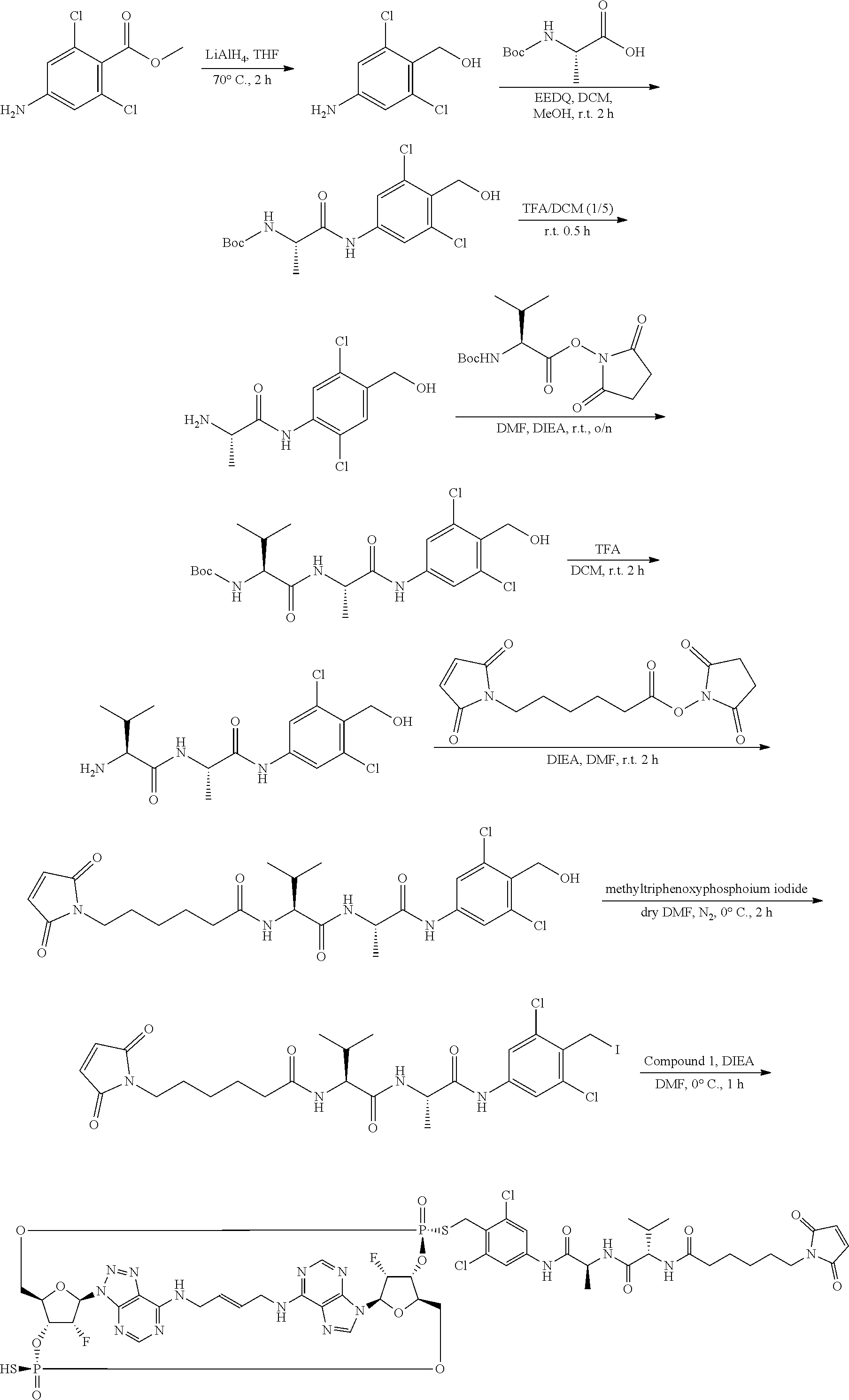

### Synthesis of (4-amino-2,6-dichlorophenyl)methanol

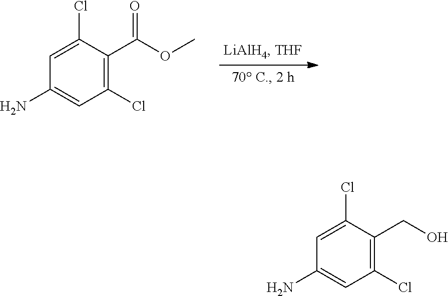

To a stirred mixture of methyl 4-amino-2,6-dichlorobenzoate (500 mg, 2.272 mmol) in THF was added LiAlH₄ (258.7 mg, 6.816 mmol) portionwise at room temperature. The resulting mixture was stirred for 2 h at 70° C. The reaction was quenched with Water/ice at 0° C. The resulting mixture was extracted with DCM. The combined organic layers were washed with water, dried over anhydrous Na₂SO₄. After filtration, the filtrate was concentrated under reduced pressure. The residue was purified by silica gel column chromatography, eluted with PE/EtOAc (5/1) to afford (4-amino-2,6-dichlorophenyl)methanol (202 mg) as a grey solid.

### Synthesis of tert-butyl N-[(1S)-1-{[3,5-dichloro-4-(hydroxymethyl)phenyl]carbamoyl}ethyl]carbamate

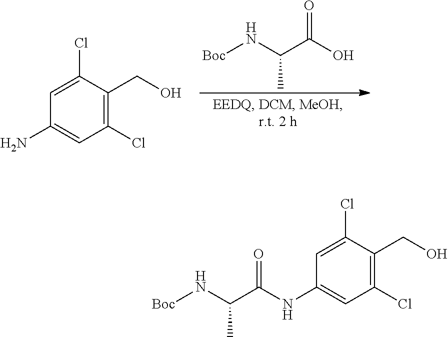 [SMILES: Nc1cc(Cl)c(CO)c(Cl)c1 ; C[C@H](NC(=O)OC(C)(C)C)C(=O)O ; C[C@H](NC(=O)OC(C)(C)C)C(=O)Nc1cc(Cl)c(CO)c(Cl)c1]

To a stirred mixture of (4-amino-2,6-dichlorophenyl)methanol (200 mg, 1.041 mmol) and 2,5-dioxopyrrolidin-1-yl (2S)-2-[(tert-butoxycarbonyl)amino]propanoate (745.4 mg, 2.603 mmol) in DCM (2.4 mL) and MeOH (0.4 mL) was added 2,5-dioxopyrrolidin-1-yl 6-(2,5-dioxo-2,5-dihydro-1H-pyrrol-1-yl)hexanoate (745.4 mg, 2.603 mmol) in portions at room temperature. The resulting mixture was stirred for 2 h at room temperature. The resulting mixture was concentrated under reduced pressure. The residue was purified by silica gel column chromatography, eluted with PE/EtOAc (5/1) to afford tert-butyl N-[(1S)-1-{[3,5-dichloro-4-(hydroxymethyl)phenyl]carbamoyl}ethyl]carbamate (320 mg) as a white solid.

### Synthesis of (2S)-2-amino-N-[3,5-dichloro-4-(hydroxymethyl)phenyl]propanamide

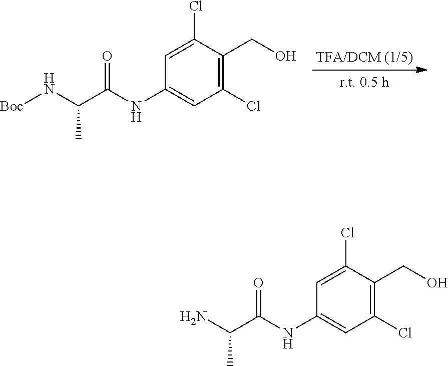 [SMILES: C[C@H](NC(=O)OC(C)(C)C)C(=O)Nc1cc(Cl)c(CO)c(Cl)c1 ; C[C@H](N)C(=O)Nc1cc(Cl)c(CO)c(Cl)c1]

A mixture of tert-butyl N-[(1S)-1-{[3,5-dichloro-4-(hydroxymethyl)phenyl]carbamoyl}ethyl]carbamate (300 mg, 0.826 mmol) and TFA (1 mL) in DCM (5 mL) was stirred for 0.5 h at room temperature. The resulting mixture was concentrated under reduced pressure to afford (2S)-2-amino-N-[3,5-dichloro-4-(hydroxymethyl)phenyl]propanamide (470 mg) as a grey oil. The crude product was used in the next step directly without further purification.

### Synthesis of tert-butyl N-[(1S)-1-{[(1S)-1-{[3,5-dichloro-4-(hydroxymethyl)phenyl]carbamoyl}ethyl]carbamoyl}-2-methylpropyl]carbamate

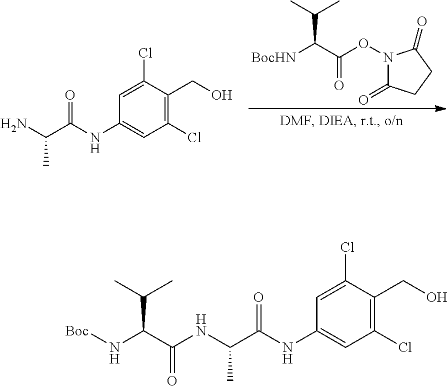 [SMILES: C[C@H](N)C(=O)Nc1cc(Cl)c(CO)c(Cl)c1 ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1cc(Cl)c(CO)c(Cl)c1]

To a stirred mixture of (2S)-2-amino-N-[3,5-dichloro-4-(hydroxymethyl)phenyl]propanamide (400 mg, 1.520 mmol) and 2,5-dioxopyrrolidin-1-yl (2S)-2-[(tert-butoxycarbonyl)amino]-3-methylbutanoate (14.3 mg, 0.046 mmol) in DMF (5 mL), Then DIEA (1964.8 mg, 15.20 mmol) was added in portions at room temperature. The resulting mixture was stirred for overnight at room temperature. Reaction was worked up and the residue was purified by silica gel column chromatography, eluted with PE/EA (5/1) to afford tert-butyl N-[(1S)-1-{[(1S)-1-{[3,5-dichloro-4-(hydroxymethyl)phenyl]carbamoyl}ethyl]carbamoyl}-2-methylpropyl]carbamate (243 mg) as a white solid.

### Synthesis of (2S)-2-amino-N-[(1S)-1-{[3,5-dichloro-4-(hydroxymethyl)phenyl]carbamoyl}ethyl]-3-methylbutan amide

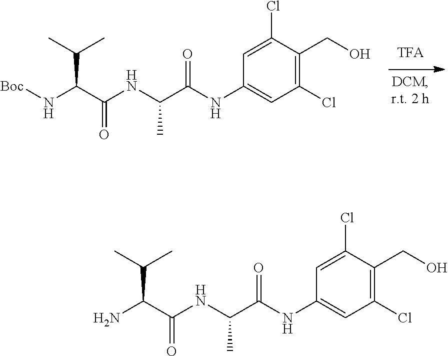 [SMILES: CC(C)[C@H](NC(=O)OC(C)(C)C)C(=O)N[C@@H](C)C(=O)Nc1cc(Cl)c(CO)c(Cl)c1 ; CC(C)[C@H](N)C(=O)N[C@@H](C)C(=O)Nc1cc(Cl)c(CO)c(Cl)c1]

A mixture of tert-butyl N-[(1S)-1-{[(1S)-1-{[3,5-dichloro-4-(hydroxymethyl)phenyl]carbamoyl}ethyl]carbamoyl}-2-methylpropyl]carbamate (230 mg, 0.497 mmol) and TFA (0.5 mL) in DCM (2.5 mL) was stirred for 2 h at room temperature. The resulting mixture was concentrated under reduced pressure. to afford (2S)-2-amino-N-[(1S)-1-{[3,5-dichloro-4-(hydroxymethyl)phenyl]carbamoyl}ethyl]-3-methylbutanamide (400 mg) as a grey oil. The crude product was used in the next step directly without further purification.

### Synthesis of N-[(1S)-1-{[(1S)-1-{[3,5-dichloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide

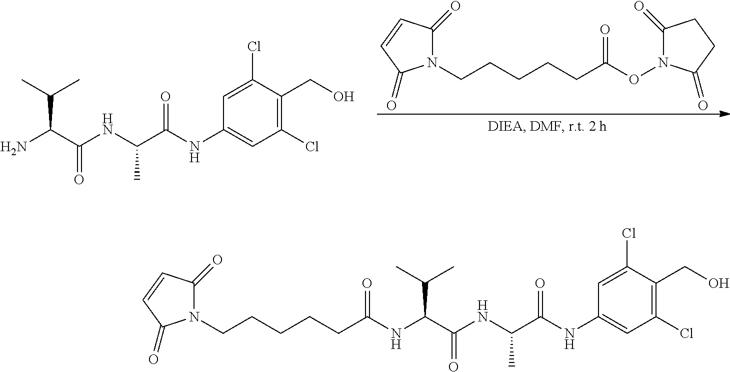 [SMILES: CC(C)[C@H](N)C(=O)N[C@@H](C)C(=O)Nc1cc(Cl)c(CO)c(Cl)c1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1cc(Cl)c(CO)c(Cl)c1]

To a stirred mixture of (2S)-2-amino-N-[(1S)-1-{[3,5-dichloro-4-(hydroxymethyl)phenyl]carbamoyl}ethyl]-3-methylbutanamide (400 mg, 1.104 mmol) and 2,5-dioxopyrrolidin-1-yl 6-(2,5-dioxo-2,5-dihydro-1H-pyrrol-1-yl)hexanoate (407.2 mg, 1.325 mmol) in DMF (4 mL) was added DIEA (1427.1 mg, 11.040 mmol) dropwise at room temperature. The resulting mixture was stirred for 2 h at room temperature. Reaction was worked up and the residue was purified by silica gel column chromatography, eluted with PE/EtOAc (5/1) to afford N-[(1S)-1-{[(1S)-1-{[3,5-dichloro-4-(hydroxymethyl)phenyl]carbamoyl}ethyl]carbamoyl}-2-methylpropyl]-6-(2,5-dioxopyrrol-1-yl)hexanamide (271 mg) as a yellow solid.

### Synthesis of N-[(1S)-1-{[(1S)-1-{[3,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide

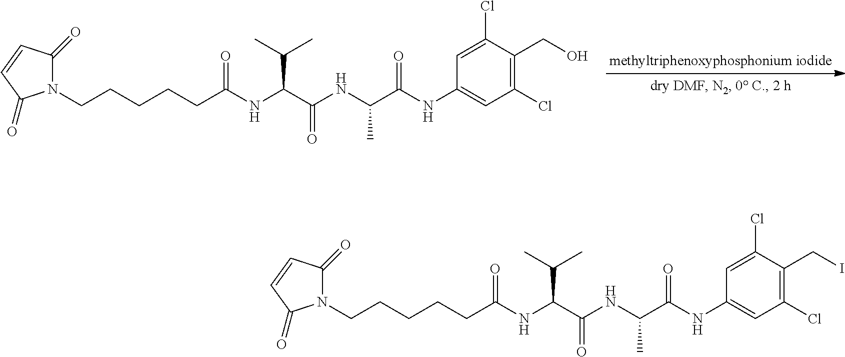 [SMILES: CC(C)[C@H](NC(=O)CCCCCN1C(=O)C=CC1=O)C(=O)N[C@@H](C)C(=O)Nc1cc(Cl)c(CO)c(Cl)c1 ; CC(C)[C@H](NC(=O)CCCCCN1C(=O)C=CC1=O)C(=O)N[C@@H](C)C(=O)Nc1cc(Cl)c(CI)c(Cl)c1]

A mixture of N-[(1S)-1-{[(1S)-1-{[3,5-dichloro-4-(hydroxymethyl)phenyl]carbamoyl}ethyl]carbamoyl}-2-methylpropyl]-6-(2,5-dioxopyrrol-1-yl)hexanamide (80 mg, 0.144 mmol) and methyl triphenoxyphosphonium iodide (195.4 mg, 0.432 mmol) in DMF (1 mL) was stirred for 2 h at 0° C. under nitrogen atmosphere. Reaction was worked up and the residue was purified by silica gel column chromatography, eluted with DCM/i-PrOH (10/1) to afford N-[(1S)-1-{[(1S)-1-{[3,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide (56 mg) as a white solid.

### Synthesis of N-[(1S)-1-{[(1S)-1-{[3,5-dichloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP47)

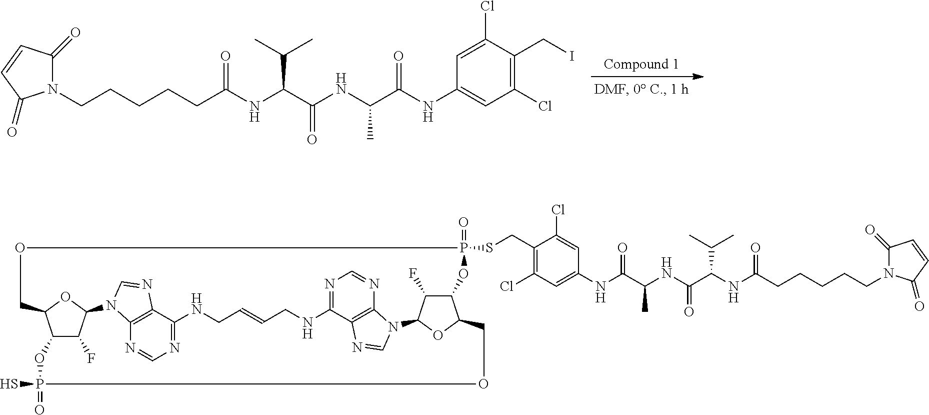 [SMILES: CC(C)[C@H](NC(=O)CCCCCN1C(=O)C=CC1=O)C(=O)N[C@@H](C)C(=O)Nc1cc(Cl)c(CI)c(Cl)c1 ; CC(C)[C@H](NC(=O)CCCCCN1C(=O)C=CC1=O)C(=O)N[C@@H](C)C(=O)Nc1cc(Cl)c(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)c(Cl)c1]

A mixture of N-[(1S)-1-{[(1S)-1-{[3,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide (99.8 mg, 0.150 mmol) and Compound 1 (56 mg, 0.075 mmol) in DMF (1 mL) was stirred for 1 h at 0° C. under nitrogen atmosphere. The crude product (56 mg) was purified by Prep-HPLC with the following conditions (Column: XBridge Prep Phenyl OBD Column, 19*150 mm, 5 μm; Mobile Phase A: Water(0.05% TFA), Mobile Phase B: MeCN; Flow rate: 60 mL/min; Gradient: 30% B to 35% B in 8 min, 35% B; Wavelength: 254 nm) to afford N-[(1S)-1-{[(1S)-1-{[3,5-dichloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP47) (9.6 mg) as a white solid.

LC-MS (ESI): [M+H]⁺: 1283.00.

### 47. Synthesis of LP48

The synthesis of LP48 is shown below:

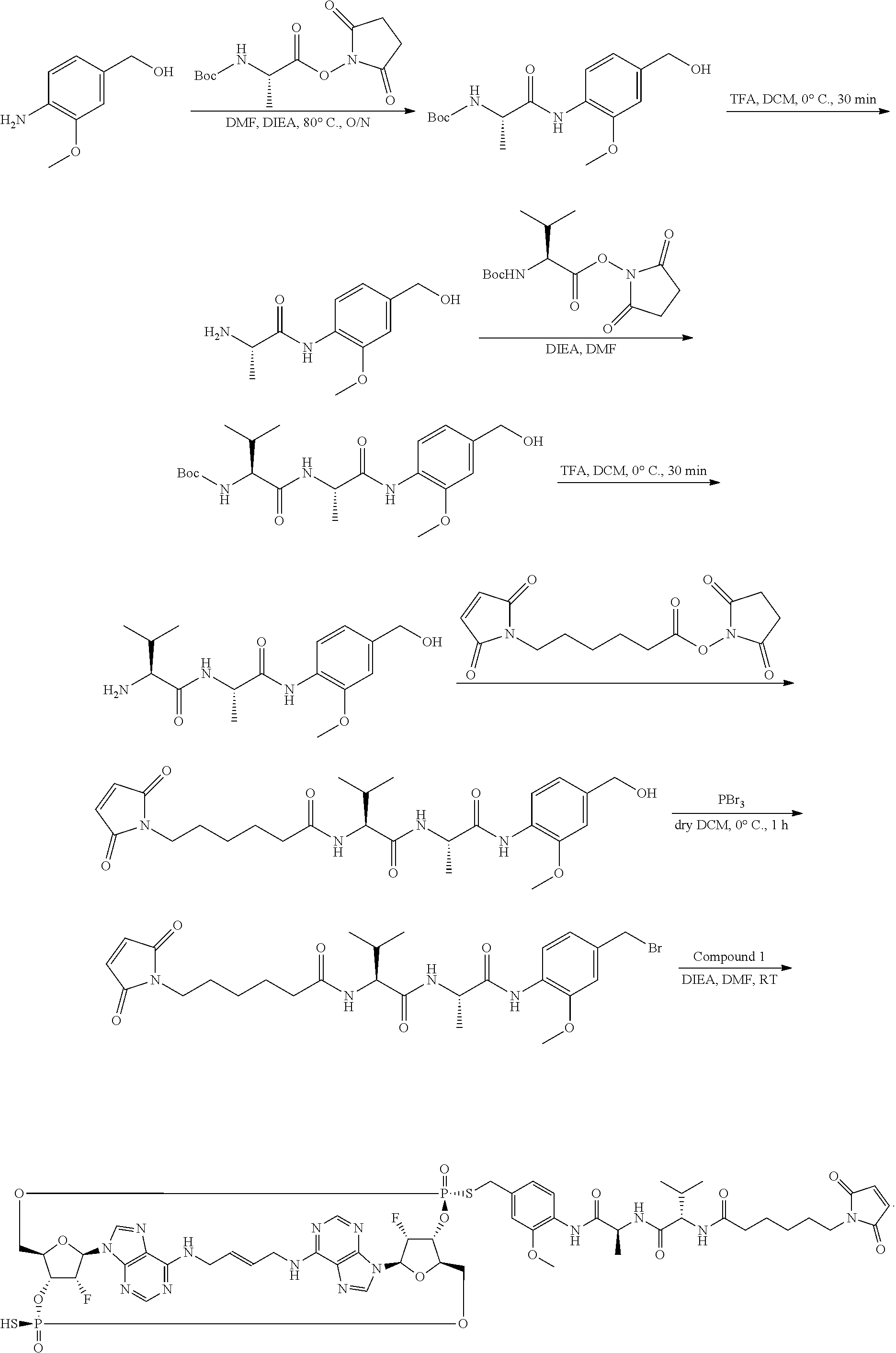

### Synthesis of tert-butyl N-[(1S)-1-{[4-(hydroxymethyl)-2-methoxyphenyl]carbamoyl}ethyl]carbamate

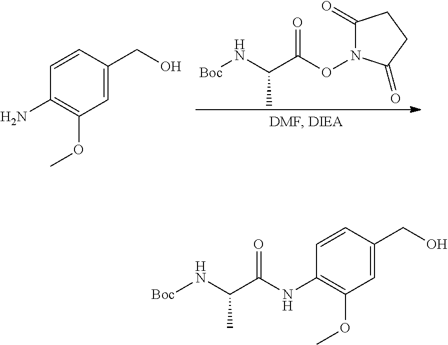

DIEA (2.53 g, 19.585 mmol) was added to a stirred mixture of 2,5-dioxopyrrolidin-1-yl (2S)-2-{[(tert-butoxy)carbonyl]amino}propanoate (8.41 g, 29.377 mmol) and (4-amino-3-methoxyphenyl)methanol (1.5 g, 9.792 mmol) in DMF (20 mL) at 25° C. The resulting mixture was stirred at 80° C. for 14 h. The resulting mixture was diluted with EtOAc. The residue was washed with water. The resulting mixture was concentrated under reduced pressure. The residue was purified by silica gel column chromatography, eluted with PE/EtOAc (3:1) to afford tert-butyl N-[(1S)-1-{[4-(hydroxymethyl)-2-methoxyphenyl]carbamoyl}ethyl]carbamate (800 mg) as a brown oil. LC-MS (ESI): 325.2 [M+H]⁺.

### Synthesis of (2S)-2-amino-N-[4-(hydroxymethyl)-2-methoxyphenyl]propanamide

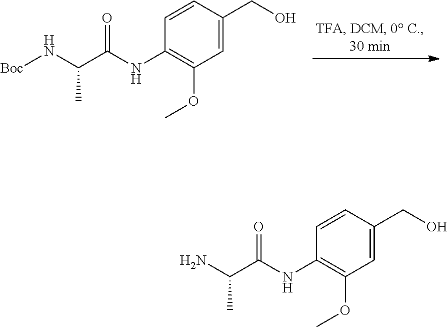 [SMILES: COc1cc(CO)ccc1NC(=O)[C@H](C)NC(=O)OC(C)(C)C ; COc1cc(CO)ccc1NC(=O)[C@H](C)N]

TFA (3 mL) was added to a stirred mixture of tert-butyl N-[(1S)-1-{[4-(hydroxymethyl)-2-methoxyphenyl]carbamoyl}ethyl]carbamate (800 mg, 2.466 mmol) in DCM (3 mL) at 0° C. The resulting mixture was stirred at 25° C. for 30 min. The resulting mixture was concentrated under reduced pressure. K₂CO₃ (1.36 g, 9.865 mmol) was added to a stirred mixture in MeOH (3 mL), THF (3 mL) and H₂O (3 mL) at 0° C. The resulting mixture was stirred at 25° C. for 2 h. The resulting mixture was concentrated under reduced pressure. The residue was purified by silica gel column chromatography, eluted with DCM/MeOH (5:1) to afford (2S)-2-amino-N-[4-(hydroxymethyl)-2-methoxyphenyl]propanamide (420 mg) as a colorless oil. LC-MS (ESI): 225.1 [M+H]⁺.

### Synthesis of tert-butyl N-[(1S)-1-{[(1S)-1-{[4-(hydroxymethyl)-2-methoxyphenyl]carbamoyl}ethyl]carbamoyl}-2-methylpropyl]carbamate

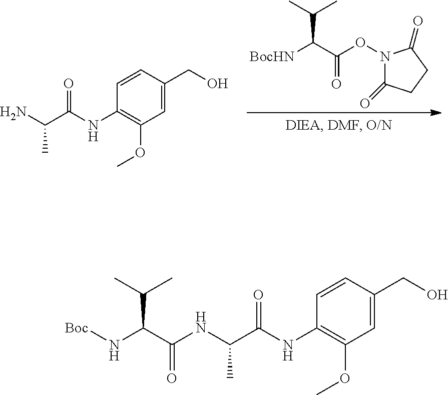 [SMILES: COc1cc(CO)ccc1NC(=O)[C@H](C)N ; CC(C)[C@H](NC(=O)OC(C)(C)C)C(=O)ON1C(=O)CCC1=O ; COc1cc(CO)ccc1NC(=O)[C@H](C)NC(=O)[C@@H](NC(=O)OC(C)(C)C)C(C)C]

DIEA (1.38 g, 10.702 mmol) was added to a stirred mixture of (2S)-2-amino-N-[4-(hydroxymethyl)-2-methoxyphenyl]propanamide (400 mg, 1.784 mmol) and 2,5-dioxopyrrolidin-1-yl (2S)-2-[(tert-butoxycarbonyl)amino]-3-methylbutanoate (560.67 mg, 1.784 mmol) in DMF (5 mL) at 25° C. The resulting mixture was stirred at 25° C. for 14 h. The resulting mixture was diluted with EtOAc. The resulting mixture was washed with water. The resulting mixture was concentrated under reduced pressure. The residue was purified by silica gel column chromatography, eluted with PE/EtOAc (1:1) to afford tert-butyl N-[(1S)-1-{[(1S)-1-{[4-(hydroxymethyl)-2-methoxyphenyl]carbamoyl}ethyl]carbamoyl}-2-methylpropyl]carbamate (470 mg) as a white solid.

LC-MS (ESI): 424.2 [M+H]⁺.

### Synthesis of (2S)-2-amino-N-[(1S)-1-{[4-(hydroxymethyl)-2-methoxyphenyl]carbamoyl}ethyl]-3-methylbutanamide

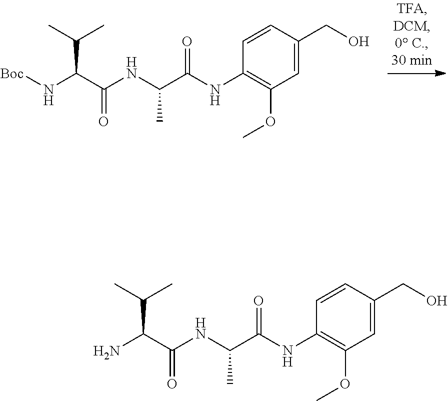 [SMILES: COc1cc(CO)ccc1NC(=O)[C@H](C)NC(=O)[C@@H](NC(=O)OC(C)(C)C)C(C)C ; COc1cc(CO)ccc1NC(=O)[C@H](C)NC(=O)[C@@H](N)C(C)C]

TFA (3 mL) was added to a stirred mixture of tert-butyl N-[(1S)-1-{[(1S)-1-{[4-(hydroxymethyl)-2-methoxyphenyl]carbamoyl}ethyl]carbamoyl}-2-methylpropyl]carbamate (450 mg, 1.063 mmol) in DCM (3 mL) at 0° C. The resulting mixture was stirred for 30 min at 25° C. The resulting mixture was concentrated under reduced pressure. K₂CO₃ (585.30 mg, 4.252 mmol) was added to a stirred mixture in MeOH (3 mL), THF (3 mL) and H₂O (3 mL) at 0° C. The resulting mixture was stirred at 25° C. for 2 h. The resulting mixture was concentrated under reduced pressure. The residue was purified by silica gel column chromatography, eluted with DCM/MeOH (6:1) to afford (2S)-2-amino-N-[(1S)-1-{[4-(hydroxymethyl)-2-methoxyphenyl]carbamoyl}ethyl]-3-methylbutanamide (270 mg) as a colorless oil. LC-MS (ESI): 324.2 [M+H]⁺.

### Synthesis of 6-(2,5-dioxo-2,5-dihydro-1H-pyrrol-1-yl)-N-[(1S)-1-{[(1S)-1-{[4-(hydroxymethyl)-2-methoxyphenyl]carbamoyl}ethyl]carbamoyl}-2-methylpropyl]hexanamide

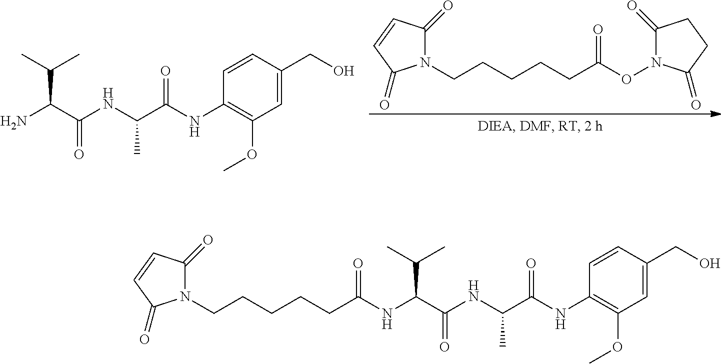

DIEA (199.82 mg, 1.546 mmol) was added to a stirred mixture of (2S)-2-amino-N-[(1S)-1-{[4-(hydroxymethyl)-2-methoxyphenyl]carbamoyl}ethyl]-3-methylbutanamide (250 mg, 0.773 mmol) and 2,5-dioxopyrrolidin-1-yl 6-(2,5-dioxo-2,5-dihydro-1H-pyrrol-1-yl)hexanoate (214.49 mg, 0.696 mmol) in DMF (3 mL) at 25° C. The resulting mixture was stirred at 25° C. for 2 h. The resulting mixture was diluted with EtOAc. The resulting mixture was washed with water. The resulting mixture was concentrated under reduced pressure. The residue was purified by silica gel column chromatography, eluted with DCM/MeOH (12:1) to afford 6-(2,5-dioxo-2,5-dihydro-1H-pyrrol-1-yl)-N-[(1S)-1-{[(1S)-1-{[4-(hydroxymethyl)-2-methoxyphenyl]carbamoyl}ethyl]carbamoyl}-2-methylpropyl]hexanamide (250 mg) as a white solid. LC-MS (ESI): 517.3 [M+H]⁺.

### Synthesis of N-[(1S)-1-{[(1S)-1-{[4-(bromomethyl)-2-methoxyphenyl]carbamoyl}ethyl]carbamoyl}-2-methylpropyl]-6-(2,5-dioxo-2,5-dihydro-1H-pyrrol-1-yl)hexanamide

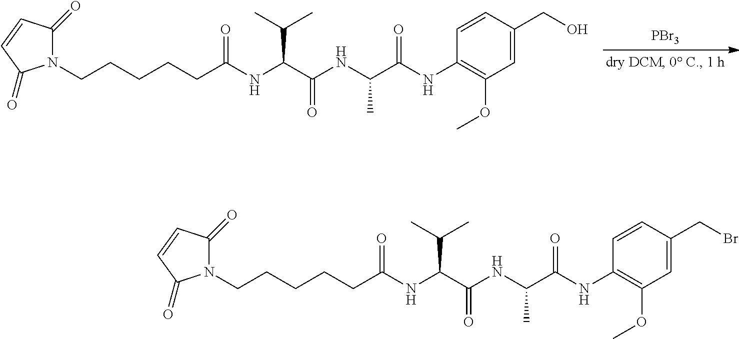 [SMILES: COc1cc(CO)ccc1NC(=O)[C@H](C)NC(=O)[C@@H](NC(=O)CCCCCN1C(=O)C=CC1=O)C(C)C ; "PBr3" ; COc1cc(CBr)ccc1NC(=O)[C@H](C)NC(=O)[C@@H](NC(=O)CCCCCN1C(=O)C=CC1=O)C(C)C]

PBr₃ (39.30 mg, 0.146 mmol) was added to a stirred mixture of 6-(2,5-dioxo-2,5-dihydro-1H-pyrrol-1-yl)-N-[(1S)-1-{[(1S)-1-{[4-(hydroxymethyl)-2-methoxyphenyl]carbamoyl}ethyl]carbamoyl}-2-methylpropyl]hexanamide (50 mg, 0.097 mmol) in DCM (2 mL) at 0° C. under nitrogen atmosphere.

The resulting mixture was stirred at 0° C. for 1 h under nitrogen atmosphere. The residue was purified by silica gel column chromatography, eluted with DCM/IPA (14:1) to afford N-[(1S)-1-{[(1S)-1-{[4-(bromomethyl)-2-methoxyphenyl]carbamoyl}ethyl]carbamoyl}-2-methylpropyl]-6-(2,5-dioxo-2,5-dihydro-1H-pyrrol-1-yl)hexanamide (50 mg) as a white solid. LC-MS (ESI): 579.2 [M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-methoxyphenyl]carbamoyl}ethyl]carbamoyl}-2-methylpropyl]-6-(2,5-dioxo-2,5-dihydro-1H-pyrrol-1-yl)hexanamide (LP48)

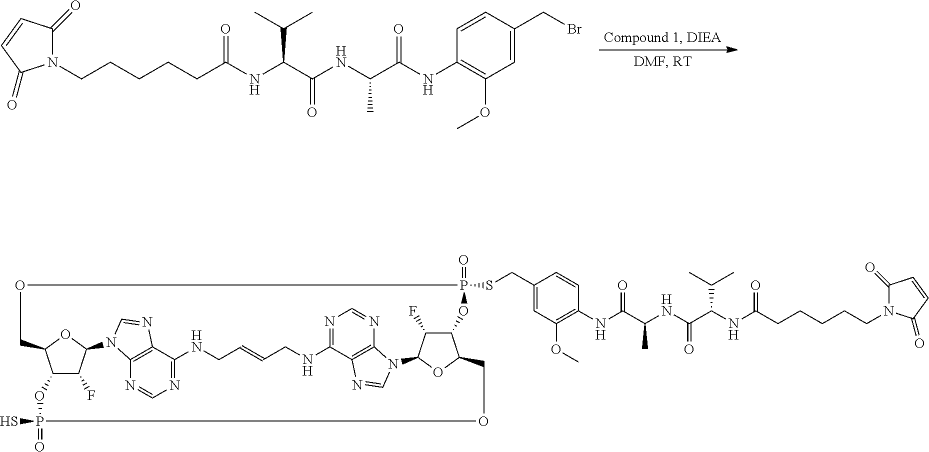 [SMILES: COc1cc(CBr)ccc1NC(=O)[C@H](C)NC(=O)[C@@H](NC(=O)CCCCCN1C(=O)C=CC1=O)C(C)C ; "Compound 1, DIEA" ; COc1cc(CS[P@@]2(=O)OC[C@H]3O[C@@H]4[C@H](F)[C@@H]3O[P@](=O)(S)OC[C@H]3O[C@H]([C@H](F)[C@@H]3O2)n2cnc3c(ncnc32)NC/C=C/CNc2ncnc3c2ncn34)ccc1NC(=O)[C@H](C)NC(=O)[C@@H](NC(=O)CCCCCN1C(=O)C=CC1=O)C(C)C]

DIEA (27.70 mg, 0.214 mmol) was added to a stirred mixture of N-[(1S)-1-{[(1S)-1-{[4-(bromomethyl)-2-methoxyphenyl]carbamoyl}ethyl]carbamoyl}-2-methylpropyl]-6-(2,5-dioxo-2,5-dihydro-1H-pyrrol-1-yl)hexanamide (49.68 mg, 0.086 mmol) and Compound 1 (32 mg, 0.043 mmol) in DMF (2 mL) at 0° C. The resulting mixture was stirred at 25° C. for 2 h. The mixture was purified by Prep-HPLC with the following conditions (Column: XBridge Prep OBD C18 Column, 19*250 mm, 5 μm; Mobile Phase A: Water(0.1% FA), Mobile Phase B: MeCN; Flow rate: 25 mL/min; Gradient: 20% B to 40% B in 12 min, 40% B; Wavelength: 254 nm) to afford N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-methoxyphenyl]carbamoyl}ethyl]carbamoyl}-2-methylpropyl]-6-(2,5-dioxo-2,5-dihydro-1H-pyrrol-1-yl)hexanamide (LP48) (8.9 mg) as a white solid.

LC-MS (ESI): 1245.80 [M+H]⁺.

### 48. Synthesis of LP49

The synthesis of LP49 is shown below:

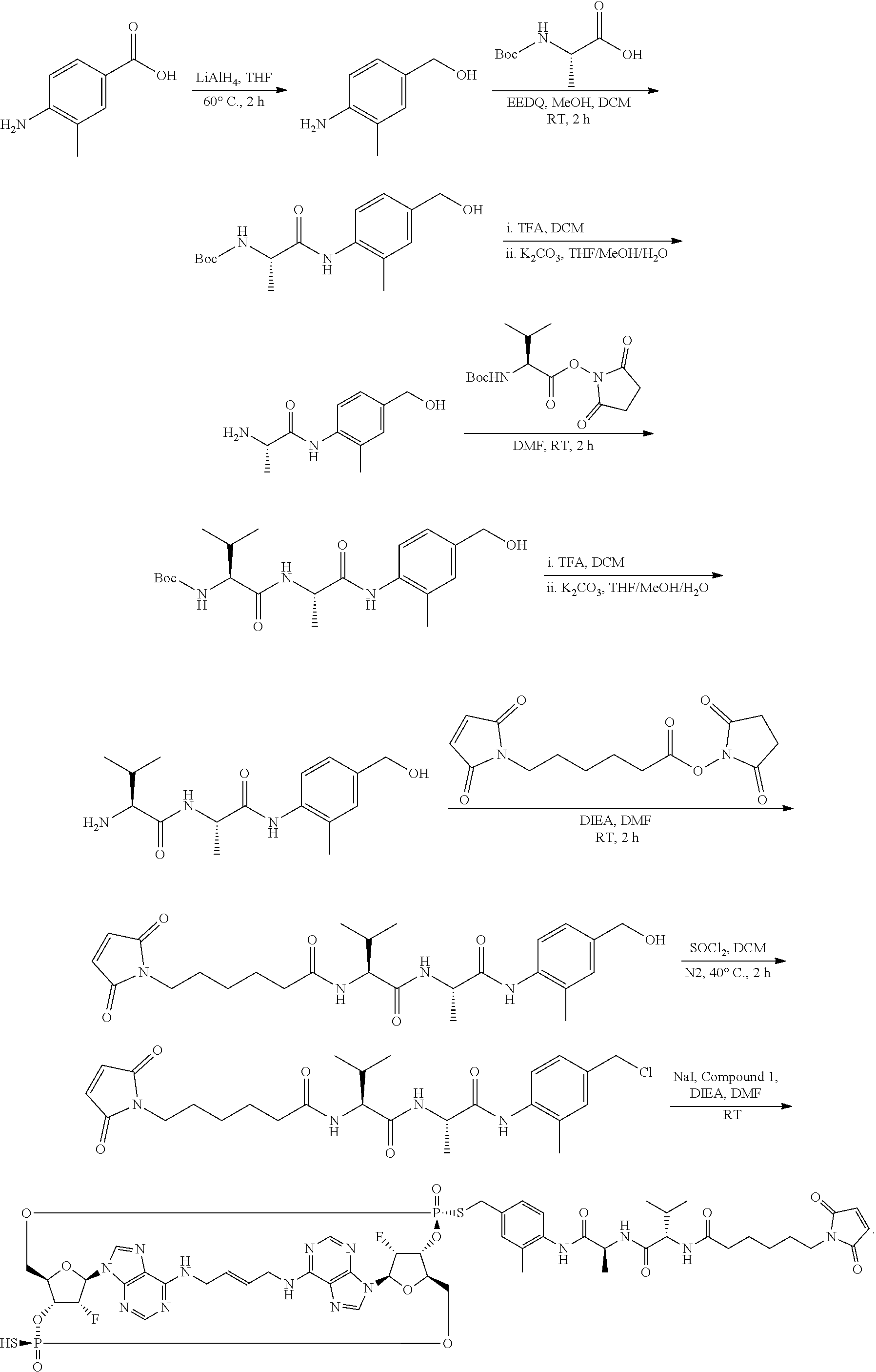

### Synthesis of (4-amino-3-methylphenyl)methanol

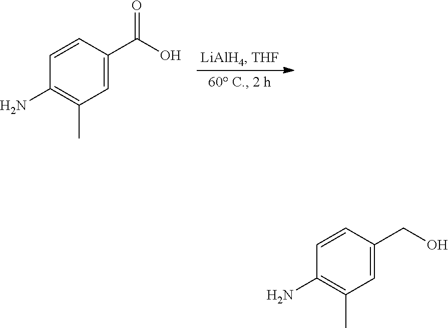

LiAlH₄ (59.54 mL, 59.538 mmol) was added to a stirred mixture of 4-amino-3-methylbenzoic acid (3 g, 19.846 mmol) in THF (50 mL) at 0° C. The resulting mixture was stirred at 60° C. for 2 h under nitrogen atmosphere. The reaction was quenched by the addition of MeOH (50 mL) at 0° C. The resulting mixture was concentrated under reduced pressure. The residue was purified by silica gel column chromatography, eluted with PE/EtOAc (1:1) to afford (4-amino-3-methylphenyl)methanol (680 mg) as a yellow solid. LC-MS (ESI): 138.09[M+H]⁺.

### Synthesis of tert-butyl N-[(1S)-1-{[4-(hydroxymethyl)-2-methylphenyl]carbamoyl}ethyl]carbamate

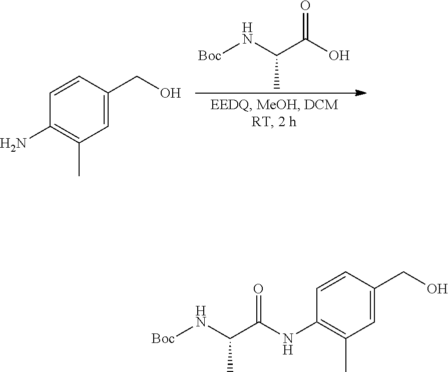

EEDQ (3.61 g, 14.580 mmol) and (2S)-2-[(tert-butoxycarbonyl)amino]propanoic acid (1.38 g, 7.290 mmol) were added to a stirred mixture of (4-amino-3-methylphenyl)methanol (1 g, 7.290 mmol) in MeOH (2 mL) and DCM (12 mL). The resulting mixture was stirred at room temperature for 2 h. The resulting mixture was concentrated under reduced pressure. The residue was purified by silica gel column chromatography, eluted with PE/EtOAc (1:1) to afford tert-butyl N-[(1S)-1-{[4-(hydroxymethyl)-2-methylphenyl]carbamoyl}ethyl]carbamate (1.1 g) as a white solid. LC-MS (ESI): 309.18[M+H]⁺.

### Synthesis of (2S)-2-amino-N-[4-(hydroxymethyl)-2-methylphenyl]propanamide

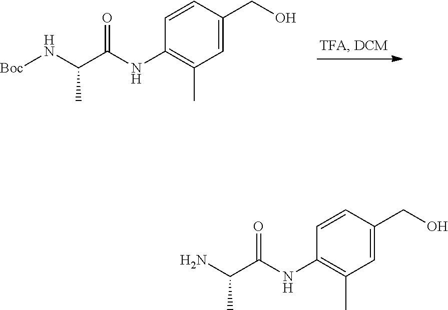 [SMILES: Cc1cc(CO)ccc1NC(=O)[C@H](C)NC(=O)OC(C)(C)C ; Cc1cc(CO)ccc1NC(=O)[C@H](C)N]

TFA (3 mL) was added to a stirred solution of tert-butyl N-[(1S)-1-{[4-(hydroxymethyl)-2-methylphenyl]carbamoyl}ethyl]carbamate (1.1 g, 3.567 mmol) in DCM (3 mL). The resulting mixture was stirred at room temperature for 30 min. The resulting mixture was concentrated under reduced pressure. Then K₂CO₃ (1.47 g, 10.636 mmol) in H₂O (1.5 mL), THF (1.5 mL) and MeOH (1.5 mL) was added to the resulting mixture. The resulting mixture was stirred at room temperature for 2 h. The resulting mixture was concentrated under reduced pressure. The residue was purified by silica gel column chromatography, eluted with DCM:MeOH (2:1) to afford (2S)-2-amino-N-[4-(hydroxymethyl)-2-methylphenyl]propanamide (600 mg) as a light yellow solid. LC-MS (ESI): 209.12[M+H]⁺.

### Synthesis of tert-butyl N-[(1S)-1-{[(1S)-1-{[4-(hydroxymethyl)-2-methylphenyl]carbamoyl}ethyl]carbamoyl}-2-methylpropyl]carbamate

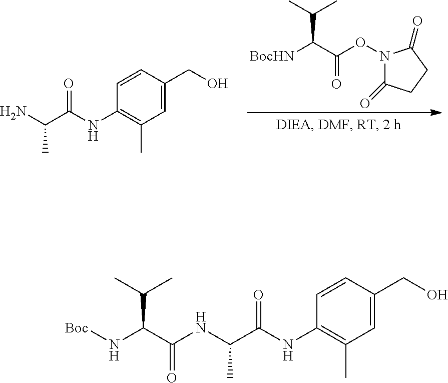 [SMILES: Cc1cc(CO)ccc1NC(=O)[C@H](C)N ; CC(C)[C@H](NC(=O)OC(C)(C)C)C(=O)ON1C(=O)CCC1=O ; Cc1cc(CO)ccc1NC(=O)[C@H](C)NC(=O)[C@@H](NC(=O)OC(C)(C)C)C(C)C]

DIEA (744.72 mg, 5.762 mmol), 2,5-dioxopyrrolidin-1-yl (2S)-2-[(tert-butoxycarbonyl)amino]-3-methylbutanoate (905.61 mg, 2.881 mmol) were added to a stirred mixture of (2S)-2-amino-N-[4-(hydroxymethyl)-2-methylphenyl]propanamide (600 mg, 2.881 mmol) in DMF (4 mL). The resulting mixture was stirred at room temperature for 2 h. The resulting mixture was diluted with EtOAc (100 mL). The resulting mixture was washed with 3×20 mL of water. The residue was purified by silica gel column chromatography, eluted with PE/EtOAc (1:2) to afford tert-butyl N-[(1S)-1-{[(1S)-1-{[4-(hydroxymethyl)-2-methylphenyl]carbamoyl}ethyl]carbamoyl}-2-methylpropyl]carbamate (800 mg) as a yellow solid. LC-MS (ESI): 408.25[M+H]⁺.

### Synthesis of (2S)-2-amino-N-[(1S)-1-{[4-(hydroxymethyl)-2-methylphenyl]carbamoyl}ethyl]-3-methylbutanamide

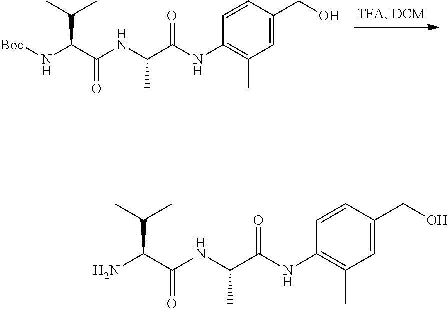 [SMILES: Cc1cc(CO)ccc1NC(=O)[C@H](C)NC(=O)[C@@H](NC(=O)OC(C)(C)C)C(C)C ; Cc1cc(CO)ccc1NC(=O)[C@H](C)NC(=O)[C@@H](N)C(C)C]

TFA (4 mL) was added to a stirred solution of tert-butyl N-[(1S)-1-{[(1S)-1-{[4-(hydroxymethyl)-2-methylphenyl]carbamoyl}ethyl]carbamoyl}-2-methylpropyl]carbamate (800 mg, 1.963 mmol) in DCM (4 mL). The resulting mixture was stirred at room temperature for 30 min. The resulting mixture was concentrated under reduced pressure. Then K₂CO₃ (1085.27 mg, 7.852 mmol) in H₂O (1.5 mL), MeOH (1.5 mL) and THF (1.5 mL) was added to the resulting mixture. The resulting mixture was stirred at room temperature for 2 h.The resulting mixture was concentrated under reduced pressure. The residue was purified by silica gel column chromatography, eluted with DCM/MeOH (1:2) to afford (2S)-2-amino-N-[(1S)-1-{[4-(hydroxymethyl)-2-methylphenyl]carbamoyl}ethyl]-3-methylbutanamide (600 mg) as a white solid. LC-MS (ESI): 309.19 [M+H]⁺.

### Synthesis of 6-(2,5-dioxo-2,5-dihydro-1H-pyrrol-1-yl)-N-[(1S)-1-{[(1S)-1-{[4-(hydroxymethyl)-2-methylphenyl]carbamoyl}ethyl]carbamoyl}-2-methylpropyl]hexanamide

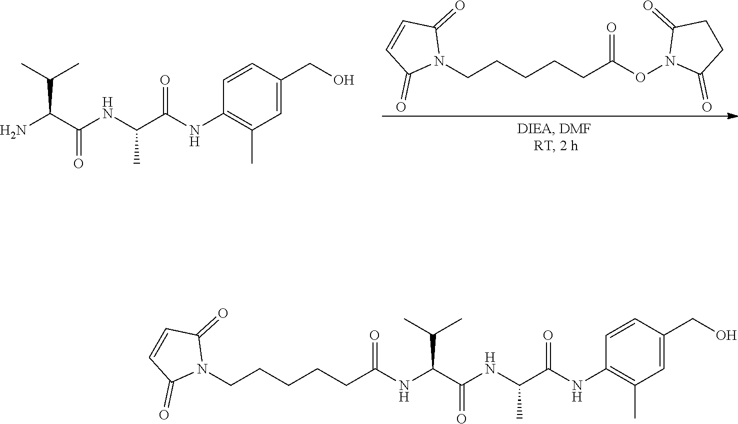 [SMILES: Cc1cc(CO)ccc1NC(=O)[C@H](C)NC(=O)[C@@H](N)C(C)C ; O=C(CCCCCN1C(=O)C=CC1=O)ON1C(=O)CCC1=O ; Cc1cc(CO)ccc1NC(=O)[C@H](C)NC(=O)[C@@H](NC(=O)CCCCCN1C(=O)C=CC1=O)C(C)C]

DIEA (496.13 mg, 3.838 mmol) and 2,5-dioxopyrrolidin-1-yl 6-(2,5-dioxo-2,5-dihydro-1H-pyrrol-1-yl)hexanoate (591.72 mg, 1.919 mmol) were added to a stirred mixture of (2S)-2-amino-N-[(1S)-1-{[4-(hydroxymethyl)-2-methylphenyl]carbamoyl}ethyl]-3-methylbutanamide (590 mg, 1.919 mmol) in DMF (3 mL). The resulting mixture was stirred at 25° C. for 2 h. The resulting mixture was diluted with EtOAc (60 mL). The resulting mixture was washed with 3×10 mL of water. The resulting mixture was concentrated under reduced pressure. The residue was purified by silica gel column chromatography, eluted with DCM/MeOH (17:1) to afford 6-(2,5-dioxo-2,5-dihydro-1H-pyrrol-1-yl)-N-[(1S)-1-{[(1S)-1-{[4-(hydroxymethyl)-2-methylphenyl]carbamoyl}ethyl]carbamoyl}-2-methylpropyl]hexanamide (380 mg) as a white solid. LC-MS (ESI): 501.27 [M+H]⁺.

### Synthesis of N-[(1S)-1-{[(1S)-1-{[4-(chloromethyl)-2-methylphenyl]carbamoyl}ethyl]carbamoyl}-2-methylpropyl]-6-(2,5-dioxo-2,5-dihydro-1H-pyrrol-1-yl)hexanamide

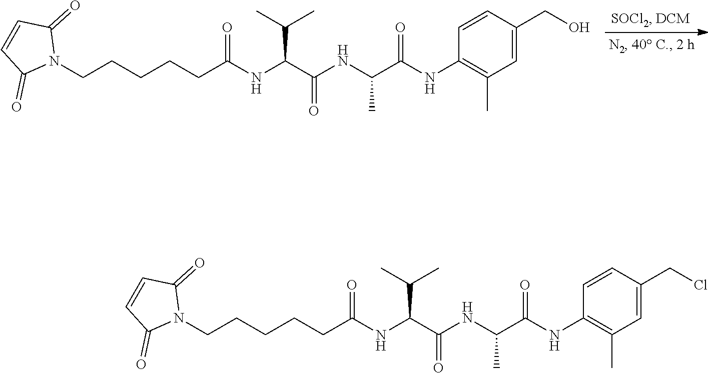 [SMILES: Cc1cc(CO)ccc1NC(=O)[C@H](C)NC(=O)[C@@H](NC(=O)CCCCCN1C(=O)C=CC1=O)C(C)C ; Cc1cc(CCl)ccc1NC(=O)[C@H](C)NC(=O)[C@@H](NC(=O)CCCCCN1C(=O)C=CC1=O)C(C)C]

SOCl₂ (42.78 mg, 0.360 mmol) was added to a stirred mixture of 6-(2,5-dioxopyrrol-1-yl)-N-[(1S)-1-{[(1S)-1-{[4-(hydroxymethyl)-2-methylphenyl]carbamoyl}ethyl]carbamoyl}-2-methylpropyl]hexanamide (90 mg, 0.180 mmol) in DCM (1 mL). The resulting mixture was stirred at 40° C. for 2 h under nitrogen atmosphere. The residue was purified by silica gel column chromatography, eluted with DCM/IPA (2:1) to afford N-[(1S)-1-{[(1S)-1-{[4-(chloromethyl)-2-methylphenyl]carbamoyl}ethyl]carbamoyl}-2-methylpropyl]-6-(2,5-dioxo-2,5-dihydro-1H-pyrrol-1-yl)hexanamide (90 mg) as a white solid. LC-MS (ESI): 519.23[M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-methylphenyl]carbamoyl}ethyl]carbamoyl}-2-methylpropyl]-6-(2,5-dioxo-2,5-dihydro-1H-pyrrol-1-yl)hexanamide (LP49)

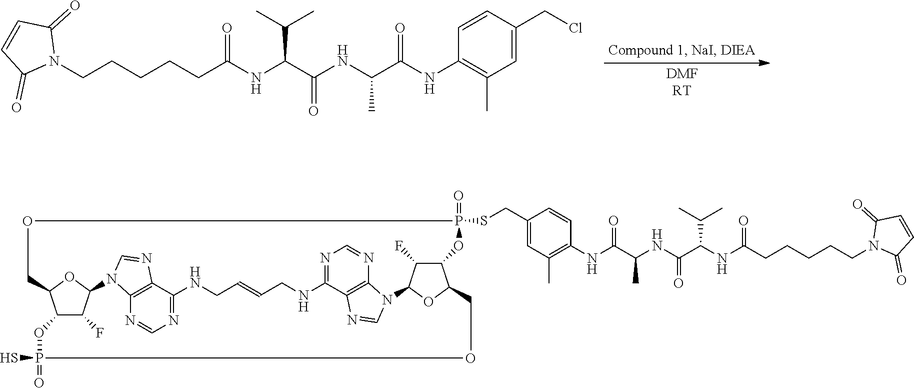

NaI (12.05 mg, 0.080 mmol) was added to a stirred mixture of N-[(1S)-1-{[(1S)-1-{[4-(chloromethyl)-2-methylphenyl]carbamoyl}ethyl]carbamoyl}-2-methylpropyl]-6-(2,5-dioxo-2,5-dihydro-1H-pyrrol-1-yl)hexanamide (65 mg, 0.125 mmol) in DMF (1.5 mL). The resulting mixture was stirred at room temperature for 1 h, then Compound 1 (60 mg, 0.080 mmol) and DIEA (51.93 mg, 0.400 mmol) were added. The solution was stirred at room temperature for 1 h. The crude product was purified by Prep-HPLC with the following conditions Column: XBridge Prep OBD C18 Column, 19*250 mm, 5 μm; Mobile Phase A: Water(0.1% FA), Mobile Phase B: MeCN; Flow rate: 25 mL/min; Gradient: 29% B to 29% B in 12 min, 29% B; Wavelength: 254 nm; RT1(min): 10.8; to afford N-[(1S)-1-{[(1S)-1-{[4-({[(1R,3R,15E,28R,29R,30R,31R,34,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2-methylphenyl]carbamoyl}ethyl]carbamoyl}-2-methylpropyl]-6-(2,5-dioxo-2,5-dihydro-1H-pyrrol-1-yl)hexanamide (LP49) (9.9 mg) as a white solid.

LC-MS (ESI): 1229.15[M+H]⁺.

### 49. Synthesis of LP50

The synthesis of LP50 is shown below:

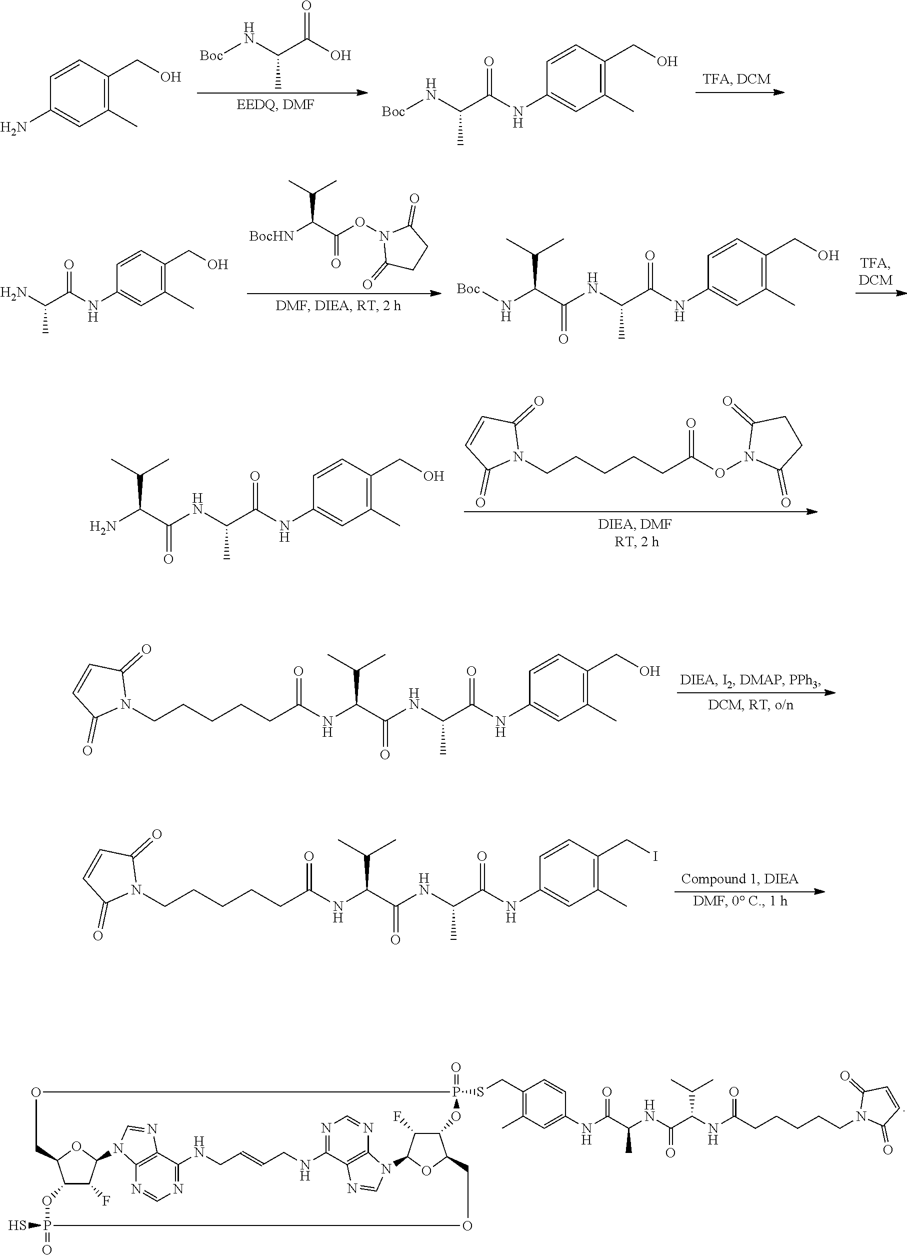

### Synthesis of tert-butyl N-[(1S)-1-{[4-(hydroxymethyl)-3-methylphenyl]carbamoyl}ethyl]carbamate

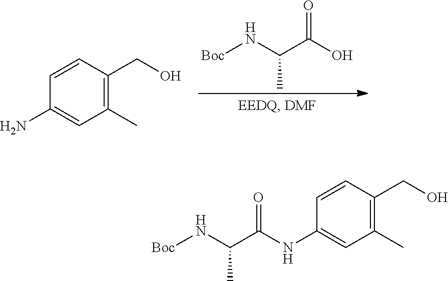

To a stirred solution of (4-amino-2-methylphenyl)methanol (782 mg, 5.700 mmol) and (2S)-2-[(tert-butoxycarbonyl)amino]propanoic acid (1294.3 mg, 6.840 mmol) in DCM (12 mL) and MeOH (2 mL) was added EEDQ (2819.3 mg, 11.400 mmol) in portions at room temperature. The resulting mixture was stirred at room temperature overnight. The resulting mixture was concentrated under vacuum. The residue was purified by silica gel column chromatography, eluted with PE/EtOAc (60/40) to afford tert-butyl N-[(1S)-1-{[4-(hydroxymethyl)-3-methylphenyl]carbamoyl}ethyl]carbamate (1.7 g) as a white solid.

### Synthesis of (2S)-2-amino-N-[4-(hydroxymethyl)-3-methylphenyl]propanamide

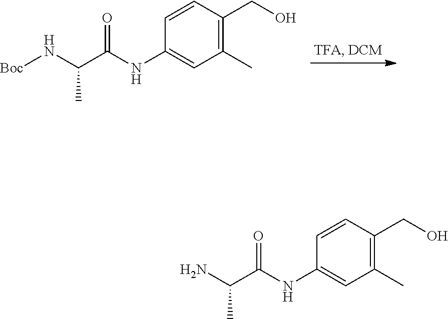 [SMILES: Cc1cc(NC(=O)[C@H](C)NC(=O)OC(C)(C)C)ccc1CO ; Cc1cc(NC(=O)[C@H](C)N)ccc1CO]

To a stirred solution of tert-butyl N-[(1S)-1-{[3-fluoro-4-(hydroxymethyl)phenyl]carbamoyl}ethyl]carbamate (850 mg, 2.721 mmol) in DCM (10 mL) was added TFA (2 mL) dropwise at room temperature. The resulting mixture was stirred for 2 h at room temperature. The resulting mixture was concentrated under vacuum. This resulted in (2S)-2-amino-N-[3-fluoro-4-(hydroxymethyl)phenyl]propanamide (815 mg) as a light yellow oil.

### Synthesis of tert-butyl N-[(1S)-1-{[(1S)-1-{[4-(hydroxymethyl)-3-methylphenyl]carbamoyl}ethyl]carbamoyl}-2-methylpropyl]carbamate

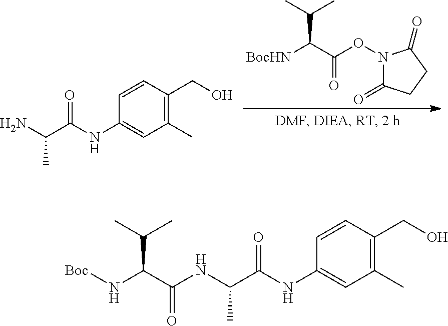 [SMILES: Cc1cc(NC(=O)[C@H](C)N)ccc1CO ; CC(C)[C@H](NC(=O)OC(C)(C)C)C(=O)ON1C(=O)CCC1=O ; Cc1cc(NC(=O)[C@H](C)NC(=O)[C@@H](NC(=O)OC(C)(C)C)C(C)C)ccc1CO]

To a stirred solution of (2S)-2-amino-N-[3-fluoro-4-(hydroxymethyl)phenyl]propanamide (448 mg, 2.151 mmol) and 2,5-dioxopyrrolidin-1-yl (2S)-2-[(tert-butoxycarbonyl)amino]-3-methylbutanoate (1328.2 mg, 4.225 mmol) in DMF (5 mL) was added DIEA (2780.2 mg, 21.510 mmol) dropwise at room temperature. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The resulting mixture was concentrated under vacuum. The residue was purified by silica gel column chromatography, eluted with PE/EtOAc (70/30) to afford tert-butyl N-[(1S)-1-{[(1S)-1-{[4-(hydroxymethyl)-3-methylphenyl]carbamoyl}ethyl]carbamoyl}-2-methylpropyl]carbamate (601 mg) as a light yellow solid.

### Synthesis of (2S)-2-amino-N-[(1S)-1-{[4-(hydroxymethyl)-3-methylphenyl]carbamoyl}ethyl]-3-methylbutanamide

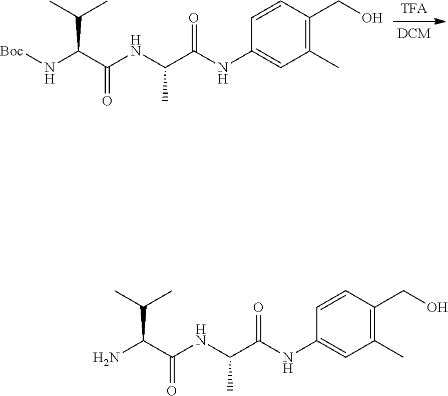 [SMILES: Cc1cc(NC(=O)[C@H](C)NC(=O)[C@@H](NC(=O)OC(C)(C)C)C(C)C)ccc1CO ; Cc1cc(NC(=O)[C@H](C)NC(=O)[C@@H](N)C(C)C)ccc1CO]

To a stirred solution of tert-butyl N-[(1S)-1-{[(1S)-1-{[4-(hydroxymethyl)-3-methylphenyl]carbamoyl}ethyl]carbamoyl}-2-methylpropyl]carbamate (601 mg, 1.475 mmol) in DCM (6 mL) was added TFA (2.0 mL) dropwise at room temperature. The resulting mixture was stirred for 1 h at room temperature. The resulting mixture was concentrated under vacuum. This resulted in (2S)-2-amino-N-[(1S)-1-{[4-(hydroxymethyl)-3-methylphenyl]carbamoyl}ethyl]-3-methylbutanamide (245 mg) as a light yellow oil.

### Synthesis of 6-(2,5-dioxo-2,5-dihydro-1H-pyrrol-1-yl)-N-[(1S)-1-{[(1S)-1-{[4-(hydroxymethyl)-3-methylphenyl]carbamoyl}ethyl]carbamoyl}-2-methylpropyl]hexanamide

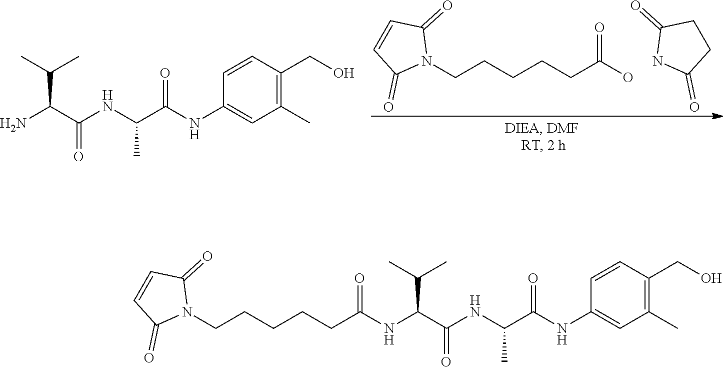

To a stirred (2S)-2-amino-N-[(1S)-1-{[4-(hydroxymethyl)-3-methylphenyl]carbamoyl}ethyl]-3-methylbutanamide (400 mg, 1.303 mmol) in DMF (5 mL) were added 2,5-dioxopyrrolidin-1-yl 6-(2,5-dioxo-2,5-dihydro-1H-pyrrol-1-yl)hexanoate (441.4 mg, 1.4332 mmol) and DIEA (1680.7 mg, 13.0290 mmol) in portions under nitrogen atmosphere. The resulting mixture was stirred for 2 h under nitrogen atmosphere. The residue was purified by silica gel column chromatography, eluted with DCM/MeOH (20/1) to afford 6-(2,5-dioxo-2,5-dihydro-1H-pyrrol-1-yl)-N-[(1S)-1-{[(1S)-1-{[4-(hydroxymethyl)-3-methylphenyl]carbamoyl}ethyl]carbamoyl}-2-methylpropyl]hexanamide (340 mg) as a white solid.

### Synthesis of 6-(2,5-dioxo-2,5-dihydro-1H-pyrrol-1-yl)-N-[(1S)-1-{[(1S)-1-{[4-(iodomethyl)-3-methylphenyl]carbamoyl}ethyl]carbamoyl}-2-methylpropyl]hexanamide

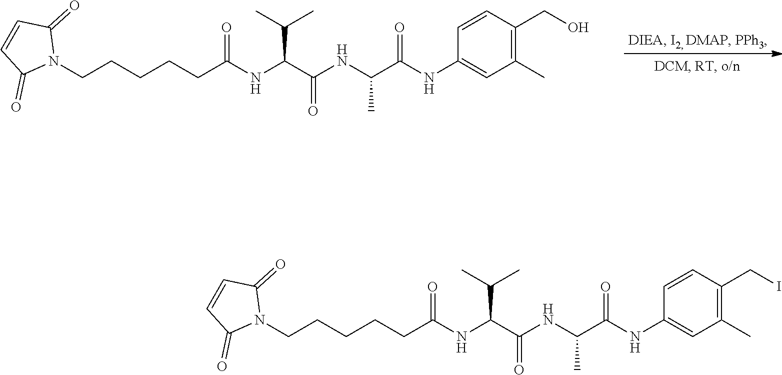 [SMILES: Cc1cc(NC(=O)[C@H](C)NC(=O)[C@@H](NC(=O)CCCCCN2C(=O)C=CC2=O)C(C)C)ccc1CO ; Cc1cc(NC(=O)[C@H](C)NC(=O)[C@@H](NC(=O)CCCCCN2C(=O)C=CC2=O)C(C)C)ccc1CI]

To a stirred solution of 6-(2,5-dioxo-2,5-dihydro-1H-pyrrol-1-yl)-N-[(1S)-1-{[(1S)-1-{[4-(hydroxymethyl)-3-methylphenyl]carbamoyl}ethyl]carbamoyl}-2-methylpropyl]hexanamide (120 mg, 0.2400 mmol) and DMAP (11.7 mg, 0.096 mmol) in DCM (1.5 mL) was added PPh₃ (94.3 mg, 0.3600 mmol), I₂ and DIEA in portions at room temperature under nitrogen atmosphere. The resulting mixture was stirred at room temperature under nitrogen atmosphere overnight. The resulting mixture was concentrated under vacuum. The residue was purified by silica gel column chromatography, eluted with DCM/IPA (95/5) to afford 6-(2,5-dioxo-2,5-dihydro-1H-pyrrol-1-yl)-N-[(1S)-1-{[(1S)-1-{[4-(iodomethyl)-3-methylphenyl]carbamoyl}ethyl]carbamoyl}-2-methylpropyl]hexanamide (20 mg) as a light yellow solid.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-3-methylphenyl]carbamoyl}ethyl]carbamoyl}-2-methylpropyl]-6-(2,5-dioxo-2,5-dihydro-1H-pyrrol-1-yl)hexanamide (LP50)

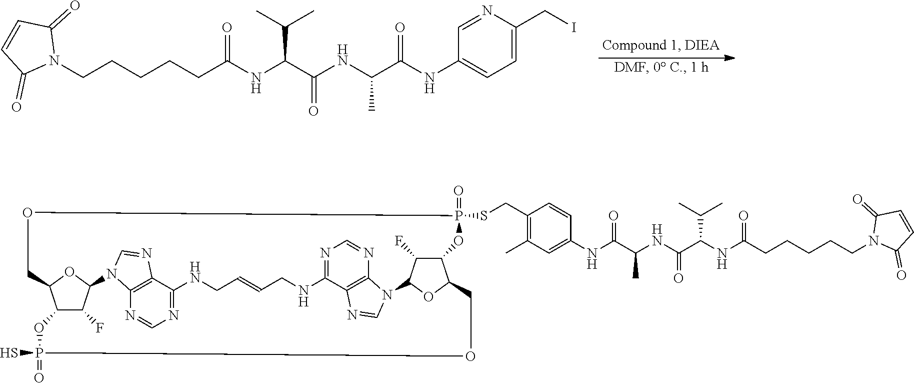

To a stirred solution of 6-(2,5-dioxo-2,5-dihydro-1H-pyrrol-1-yl)-N-[(1S)-1-{[(1S)-1-{[4-(iodomethyl)-3-methylphenyl]carbamoyl}ethyl]carbamoyl}-2-methylpropyl]hexanamide (81.8 mg, 0.134 mmol) and DIEA (43.28 mg, 0.335 mmol) in DMF (1.2 mL) was added Compound 1 (50 mg, 0.067 mmol) dropwise at 0° C. under nitrogen atmosphere. The resulting mixture was stirred for 1 h at 0° C. under nitrogen atmosphere. The crude product was purified by Prep-HPLC with the following conditions (Column: Xcelect CSH F-pheny OBD Column, 19*250 mm, 5 μm; Mobile Phase A: Water(0.1% FA), Mobile Phase B: MeCN; Flow rate: 25 mL/min; Gradient: 20% B to 40% B in 13 min, 40% B; Wavelength: 254 nm; RT1(min): 11.2). This resulted in N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-3-methylphenyl]carbamoyl}ethyl]carbamoyl}-2-methylpropyl]-6-(2,5-dioxo-2,5-dihydro-1H-pyrrol-1-yl)hexanamide (LP50) (6.8 mg) as a white solid.

LC-MS (ESI): [M+H]⁺: 1229.10.

### 50. Synthesis of LP51

The synthesis of LP51 is shown below:

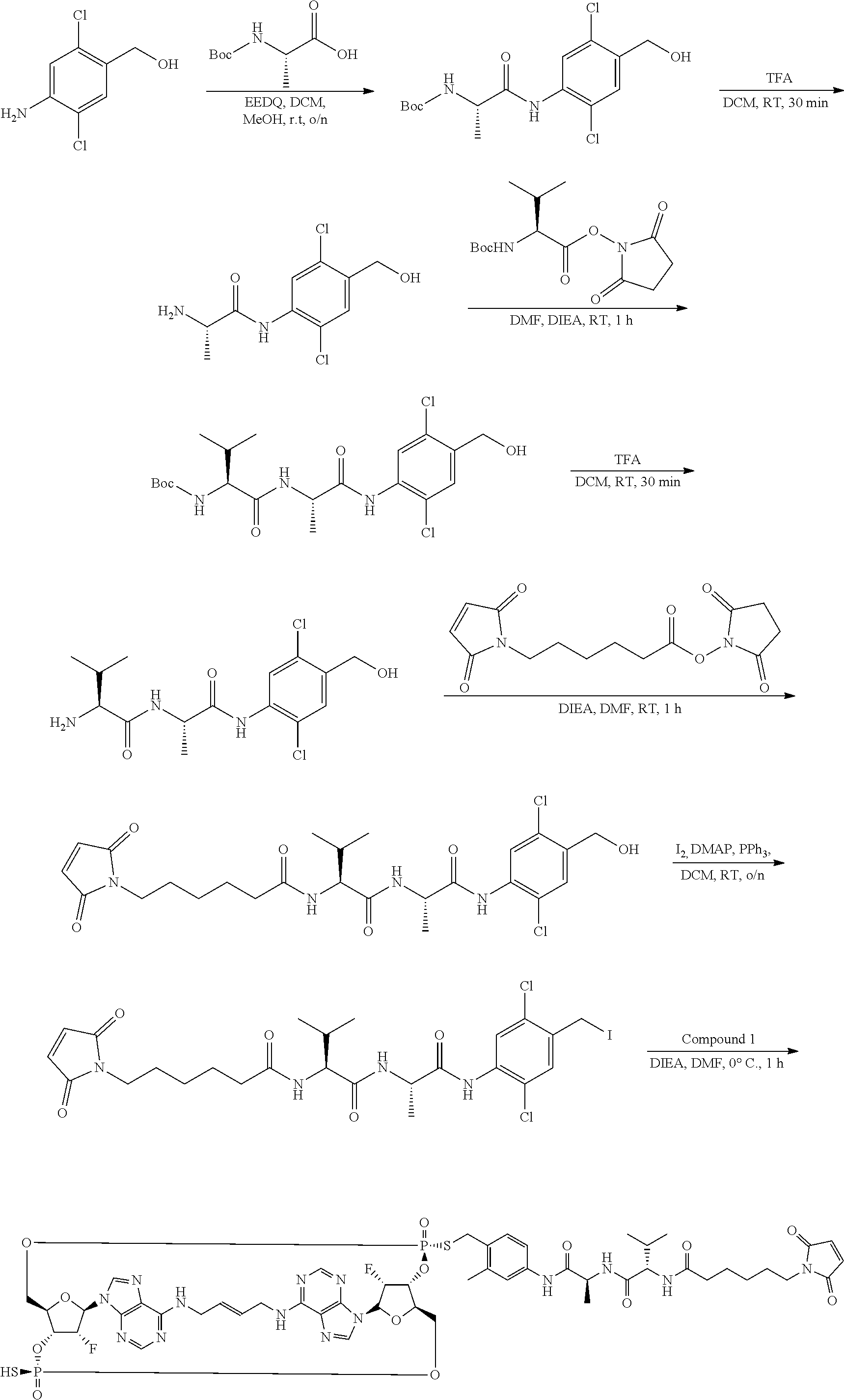

### Synthesis of tert-butyl N-[(1S)-1-{[2,5-dichloro-4-(hydroxymethyl)phenyl]carbamoyl}ethyl]carbamate

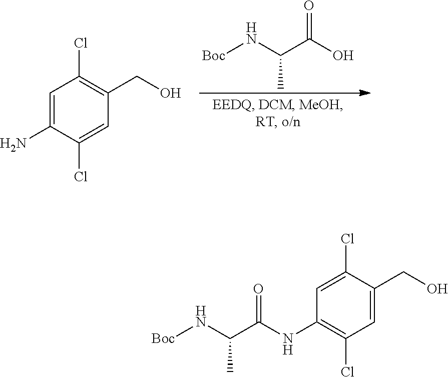 [SMILES: Nc1cc(Cl)c(CO)cc1Cl ; C[C@H](NC(=O)OC(C)(C)C)C(=O)O ; C[C@H](NC(=O)OC(C)(C)C)C(=O)Nc1cc(Cl)c(CO)cc1Cl]

A mixture of (4-amino-2,5-dichlorophenyl)methanol (500 mg, 2.604 mmol), EEDQ (1.29 g, 5.208 mmol) and (2S)-2-{[(tert-butoxy)carbonyl]amino}propanoic acid in DCM (6 mL), MeOH (1 mL) was stirred overnight at room temperature under air atmosphere. The resulting mixture was concentrated under reduced pressure. The residue was purified by silica gel column chromatography, eluted with DCM/EtOAc (3/1) to afford tert-butyl N-[(1S)-1-{[2,5-dichloro-4-(hydroxymethyl)phenyl]carbamoyl}ethyl]carbamate (248 mg) as a white solid.

### Synthesis of (2S)-2-amino-N-[2,5-dichloro-4-(hydroxymethyl)phenyl]propenamide

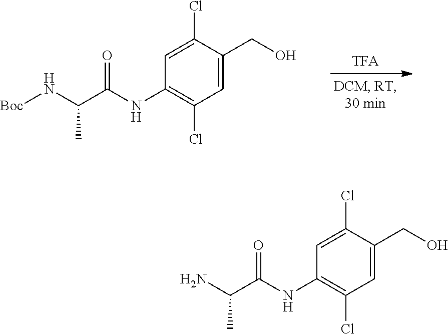 [SMILES: C[C@H](NC(=O)OC(C)(C)C)C(=O)Nc1cc(Cl)c(CO)cc1Cl ; C[C@H](N)C(=O)Nc1cc(Cl)c(CO)cc1Cl]

A solution of tert-butyl N-[(1S)-1-{[2,5-dichloro-4-(hydroxymethyl)phenyl]carbamoyl}ethyl]carbamate (238 mg, 0.655 mmol) and TFA (1 mL) in DCM (3 mL) was stirred for 30 min at room temperature. The resulting mixture was concentrated under reduced pressure to afford (2S)-2-amino-N-[2,5-dichloro-4-(hydroxymethyl)phenyl]propanamide (172 mg) as a yellow oil.

### Synthesis of tert-butyl N-[(1S)-1-{[(1S)-1-{[2,5-dichloro-4-(hydroxymethyl)phenyl]carbamoyl}ethyl]carbamoyl}-2-methylpropyl]carbamate

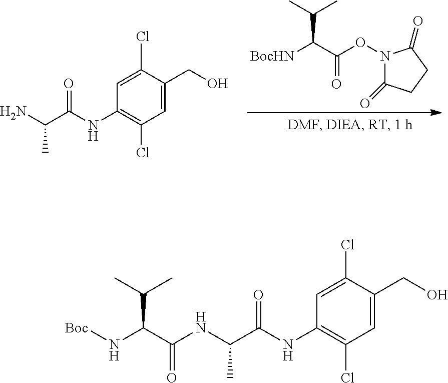 [SMILES: C[C@H](N)C(=O)Nc1cc(Cl)c(CO)cc1Cl ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1cc(Cl)c(CO)cc1Cl]

A mixture of (2S)-2-amino-N-[2,5-dichloro-4-(hydroxymethyl)phenyl]propanamide (150 mg, 0.570 mmol), DIEA (736.79 mg, 5.700 mmol), and 2,5-dioxopyrrolidin-1-yl (2S)-2-{[(tert-butoxy)carbonyl]amino}-3-methylbutanoate in DMF (2 mL) was stirred for 1 h at room temperature. Reaction was worked up and the residue was purified by silica gel column chromatography, eluted with PE/EtOAc (5/1) to afford tert-butyl N-[(1S)-1-{[(1S)-1-{[2,5-dichloro-4-(hydroxymethyl)phenyl]carbamoyl}ethyl]carbamoyl}-2-methylpropyl]carbamate (171 mg) as a yellow oil.

### Synthesis of (2S)-2-amino-N-[(1S)-1-{[2,5-dichloro-4-(hydroxymethyl)phenyl]carbamoyl}ethyl]-3-methylbutanamide

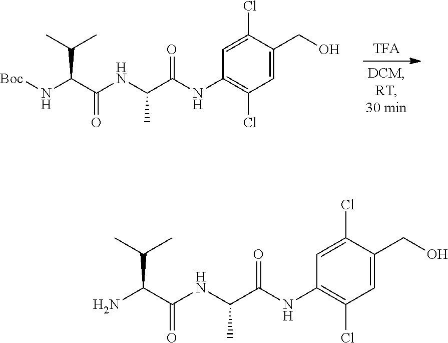 [SMILES: CC(C)[C@H](NC(=O)OC(C)(C)C)C(=O)N[C@@H](C)C(=O)Nc1cc(Cl)c(CO)cc1Cl ; CC(C)[C@H](N)C(=O)N[C@@H](C)C(=O)Nc1cc(Cl)c(CO)cc1Cl]

A solution of tert-butyl N-[(1S)-1-{[(1S)-1-{[2,5-dichloro-4-(hydroxymethyl)phenyl]carbamoyl}ethyl]carbamoyl}-2-methylpropyl]carbamate (160 mg, 0.346 mmol) and TFA (1 mL) in DCM (3 mL) was stirred for 30 min at room temperature. The resulting mixture was concentrated under reduced pressure to afford (2S)-2-amino-N-[(1S)-1-{[2,5-dichloro-4-(hydroxymethyl)phenyl]carbamoyl}ethyl]-3-methylbutanamide (100 mg) as a yellow oil.

### Synthesis of N-[(1S)-1-{[(1S)-1-{[2,5-dichloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide

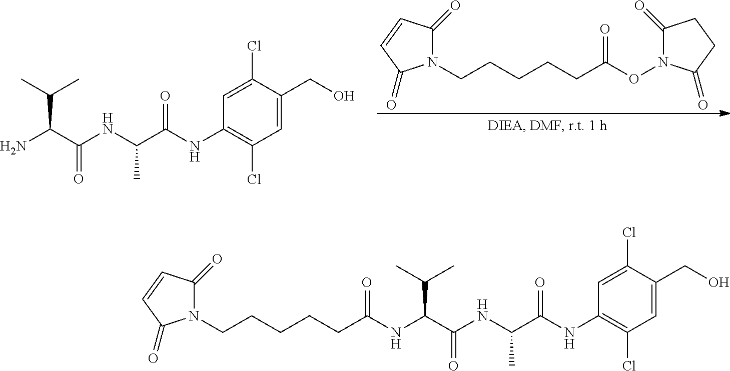 [SMILES: CC(C)[C@H](N)C(=O)N[C@@H](C)C(=O)Nc1cc(Cl)c(CO)cc1Cl ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1cc(Cl)c(CO)cc1Cl]

A mixture of (2S)-2-amino-N-[(1S)-1-{[2,5-dichloro-4-(hydroxymethyl)phenyl]carbamoyl}ethyl]-3-methylbutanamide (90 mg, 0.248 mmol), 2,5-dioxopyrrolidin-1-yl 6-(2,5-dioxo-2,5-dihydro-1H-pyrrol-1-yl)hexanoate (84.25 mg, 0.273 mmol) and DIEA (321.10 mg, 2.480 mmol) in DMF (2 mL) was stirred for 1 h at room temperature. Reaction was worked up and the residue was purified by silica gel column chromatography, eluted with DCM/MeOH (10/1) to afford N-[(1S)-1-{[(1S)-1-{[2,5-dichloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide (80 mg) as a white solid.

### Synthesis of N-[(1S)-1-{[(1S)-1-{[2,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide

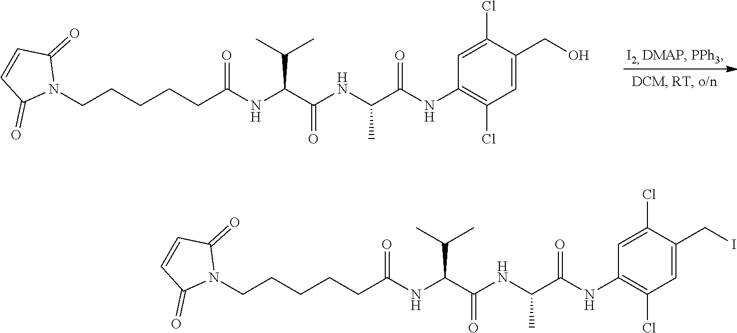 [SMILES: CC(C)[C@H](NC(=O)CCCCCN1C(=O)C=CC1=O)C(=O)N[C@@H](C)C(=O)Nc1cc(Cl)c(CO)cc1Cl ; CC(C)[C@H](NC(=O)CCCCCN1C(=O)C=CC1=O)C(=O)N[C@@H](C)C(=O)Nc1cc(Cl)c(CI)cc1Cl]

A solution of N-[(1S)-1-{[(1S)-1-{[2,5-dichloro-4-(hydroxymethyl)phenyl]carbamoyl}ethyl]carbamoyl}-2-methylpropyl]-6-(2,5-dioxo-2,5-dihydro-1H-pyrrol-1-yl)hexanamide (40 mg, 0.063 mmol), I₂ (23.93 mg, 0.095 mmol) DMAP (3.07 mg, 0.025 mmol) and PPh₃ (24.73 mg, 0.095 mmol) in DCM (2 mL) was stirred for overnight at room temperature. The resulting mixture was concentrated under reduced pressure. The residue was purified by silica gel column chromatography, eluted with DCM/IPA (10/1) to afford N-[(1S)-1-{[(1S)-1-{[2,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide (35 mg) as a white solid.

### Synthesis of N-[(1S)-1-{[(1S)-1-{[2,5-dichloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP51)

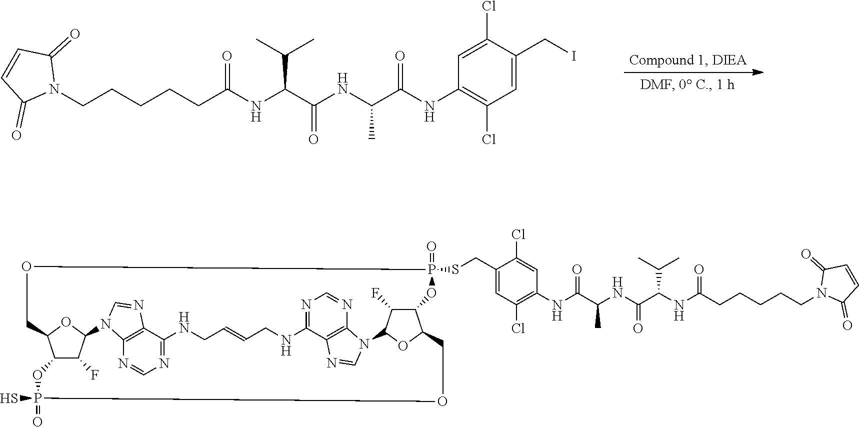

A mixture of N-[(1S)-1-{[(1S)-1-{[2,5-dichloro-4-(iodomethyl)phenyl]carbamoyl}ethyl]carbamoyl}-2-methylpropyl]-6-(2,5-dioxo-2,5-dihydro-1H-pyrrol-1-yl)hexanamide (73.12 mg, 0.108 mmol), Compound 1 (40 mg, 0.054 mmol) and DIEA (34.62 mg, 0.270 mmol) in DMF (1 mL) was stirred for 1 h at 0° C. under nitrogen atmosphere. The crude product was purified by Prep-HPLC with the following conditions (Column: XBridge Shield RP18 OBD Column, 19*250 mm, 10 μm; Mobile Phase A: Water(10 mmol/L NH₄HCO₃), Mobile Phase B: MeCN; Flow rate: 25 mL/min; Gradient: 32% B to 32% B in 13 min, 32% B; Wavelength: 254 nm; RT1(min): 11.5) to afford N-[(1S)-1-{[(1S)-1-{[2,5-dichloro-4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP51) (5.5 mg) as a white solid.

LC-MS (ESI): [M+H]⁺: 1282.95.

### 51. Synthesis of LP52

The synthesis of LP52 is shown below:

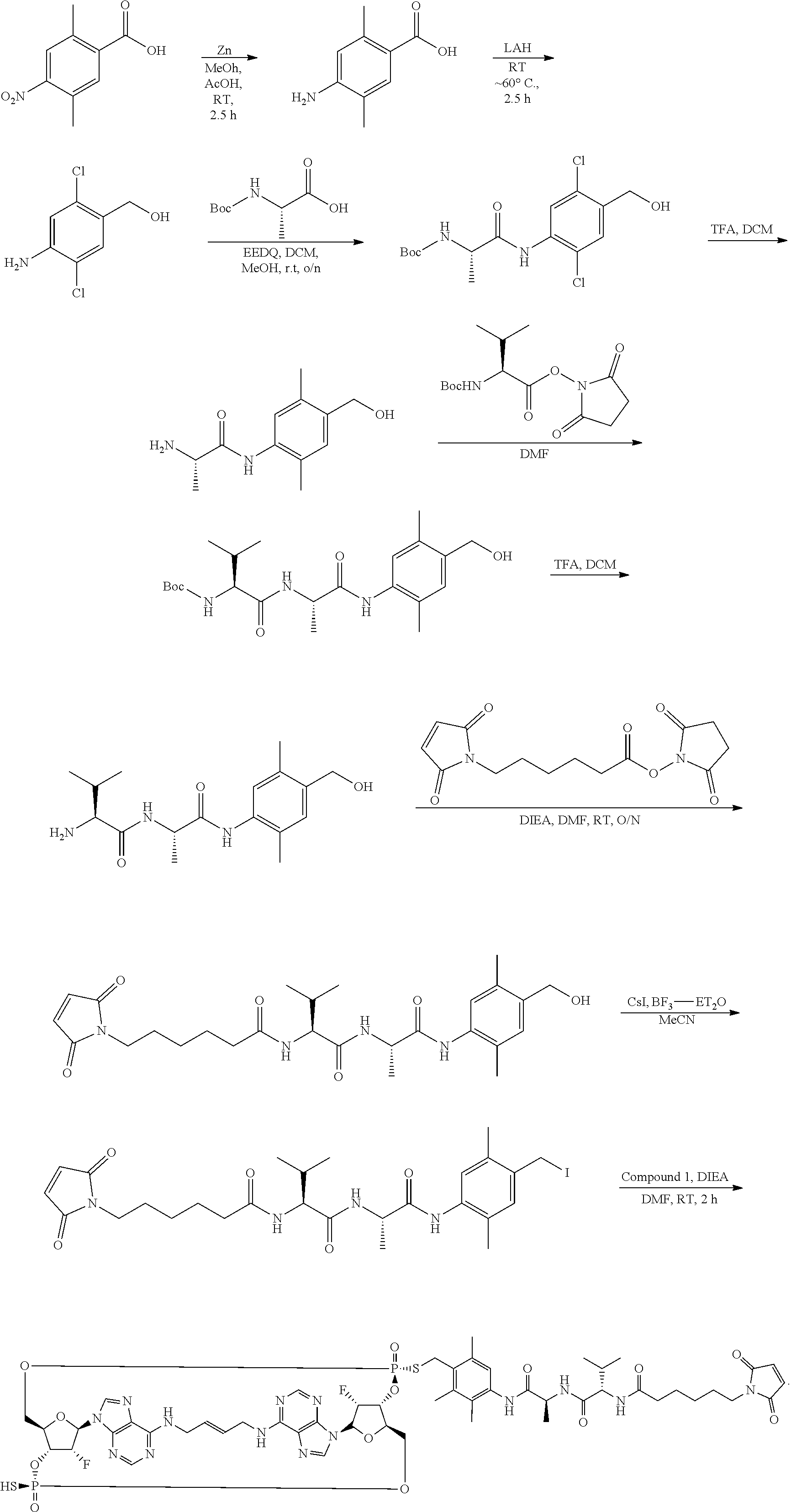

### Synthesis of 4-amino-2,5-dimethylbenzoic acid

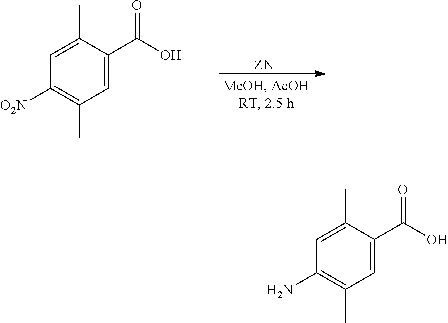

Zn (3.4 g, 51.235 mmol) was added to a mixture of 2,5-dimethyl-4-nitrobenzoic acid (2 g, 10.247 mmol) in MeOH (15 mL) and AcOH (3 mL). The resulting mixture was stirred at 25° C. for 16 h. The resulting mixture was concentrated under reduced pressure. The resulting mixture was diluted with EtOAc, washed with water, and concentrated. The residue was purified by silica gel column chromatography, eluted with PE/EtOAc (1:1). This resulted in 1.1 g of 4-amino-2,5-dimethylbenzoic acid as an off-white solid. LC-MS (ESI): 166 [M+H]⁺.

### Synthesis of (4-amino-2,5-dimethylphenyl)methanol

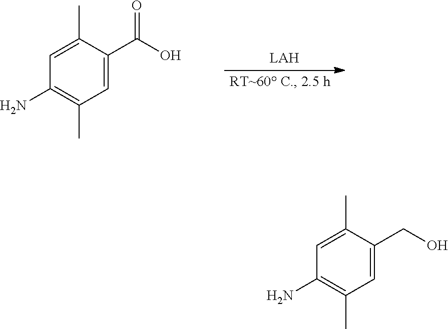

LAH (13.3 ml, 13.318 mmol) was added to a mixture of 4-amino-2,5-dimethylbenzoic acid (1.1 g, 6.659 mmol) in THF (20 mL) at 0° C. The resulting mixture was stirred at 60° C. for 2.5 h. The reaction was quenched by the addition of MeOH (10 mL) at 0° C. The resulting mixture was concentrated under reduced pressure. The residue was purified by silica gel column chromatography, eluted with CH₂Cl₂/MeOH (10:1). This resulted in 500 mg of (4-amino-2,5-dimethylphenyl)methanol as a yellow solid. LC-MS (ESI): 152 [M+H]⁺.

### Synthesis of tert-butyl N-[(1S)-1-{[4-(hydroxymethyl)-2,5-dimethylphenyl]carbamoyl}ethyl]carbamate

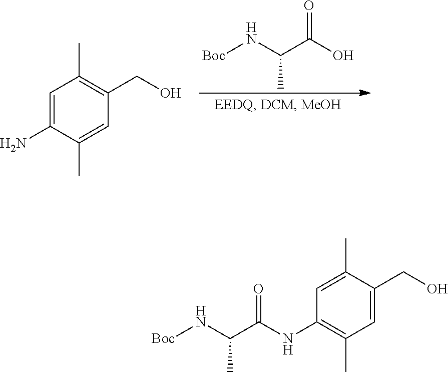 [SMILES: Cc1cc(CO)c(C)cc1N ; C[C@H](NC(=O)OC(C)(C)C)C(=O)O ; Cc1cc(NC(=O)[C@H](C)NC(=O)OC(C)(C)C)c(C)cc1CO]

EEDQ (1.4 g, 5.820 mmol) was added to a mixture of (4-amino-2,5-dimethylphenyl)methanol (440 mg, 2.910 mmol) and (2S)-2-[(tert-butoxycarbonyl)amino]propanoic acid (551 mg, 2.910 mmol) in DCM (6 mL) and MeOH (1 mL). The resulting mixture was stirred at 25° C. for 2 h. The resulting mixture was concentrated under reduced pressure. The residue was purified by silica gel column chromatography, eluted with PE/EtOAc (1:1). This resulted in 800 mg of tert-butyl N-[(1S)-1-{[4-(hydroxymethyl)-2,5-dimethylphenyl]carbamoyl}ethyl]carbamate as a white solid. LC-MS (ESI): 323 [M+H]⁺.

### Synthesis of (2S)-2-amino-N-[4-(hydroxymethyl)-2,5-dimethylphenyl]propanamide

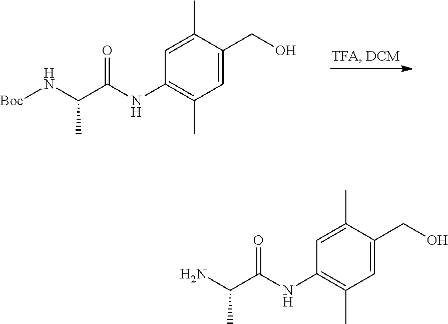 [SMILES: Cc1cc(NC(=O)[C@H](C)NC(=O)OC(C)(C)C)c(C)cc1CO ; Cc1cc(NC(=O)[C@H](C)N)c(C)cc1CO]

TFA (5 mL) was added to a mixture of tert-butyl N-[(1S)-1-{[4-(hydroxymethyl)-2,5-dimethylphenyl]carbamoyl}ethyl]carbamate (760 mg, 2.357 mmol) in DCM (5 mL) at 0° C. The resulting mixture was stirred at 25° C. for 2.5 h. The resulting mixture was concentrated under reduced pressure. To the above mixture were added K₂CO₃ (652 mg, 4.714 mmol), MeOH (4 mL), THF (4 mL) and H₂O (4 mL) dropwise at 0° C. The resulting mixture was stirred for additional 2 h at 25° C. The resulting mixture was concentrated under reduced pressure. The residue was purified by silica gel column chromatography, eluted with CH₂Cl₂/MeOH (5:1). This resulted in 460 mg of (2S)-2-amino-N-[4-(hydroxymethyl)-2,5-dimethylphenyl]propanamide as a yellow solid. LC-MS (ESI): 223 [M+H]⁺.

### Synthesis of tert-butyl N-[(1S)-1-{[(1S)-1-{[4-(hydroxymethyl)-2,5-dimethylphenyl]carbamoyl}ethyl]carbamoyl}-2-methylpropyl]carbamate

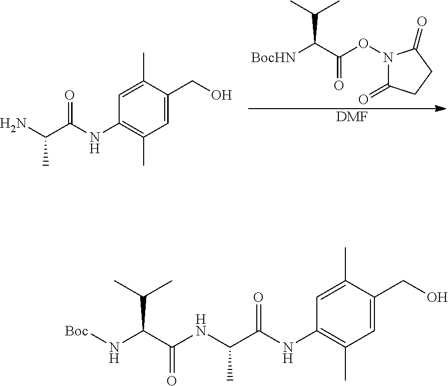

2,5-Dioxopyrrolidin-1-yl (2S)-2-[(tert-butoxycarbonyl)amino]-3-methylbutanoate (650 mg, 2.069 mmol) was added to a mixture of (2S)-2-amino-N-[4-(hydroxymethyl)-2,5-dimethylphenyl]propanamide (460 mg, 2.069 mmol) in DMF (5 mL). The resulting mixture was stirred at 25° C. for 2 h. The resulting mixture was diluted with EtOAc, washed with water, and concentrated. The residue was purified by silica gel column chromatography, eluted with PE/EtOAc (1:2). This resulted in tert-butyl N-[(1S)-1-{[(1S)-1-{[4-(hydroxymethyl)-2,5-dimethylphenyl]carbamoyl}ethyl]carbamoyl}-2-methylpropyl]carbamate (520 mg) as a yellow solid.

LC-MS (ESI): 423 [M+H]⁺.

### Synthesis of (2S)-2-amino-N-[(1S)-1-{[4-(hydroxymethyl)-2,5-dimethylphenyl]carbamoyl}ethyl]-3-methylbutanamide

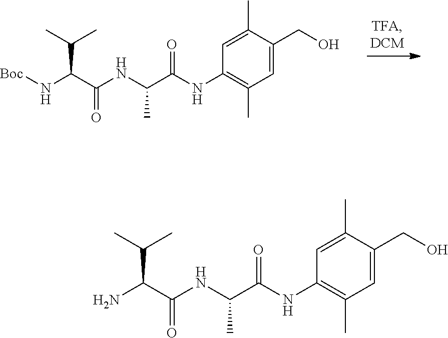 [SMILES: Cc1cc(NC(=O)[C@H](C)NC(=O)[C@@H](NC(=O)OC(C)(C)C)C(C)C)c(C)cc1CO ; Cc1cc(NC(=O)[C@H](C)NC(=O)[C@@H](N)C(C)C)c(C)cc1CO]

TFA (5 mL) was added to a mixture of tert-butyl N-[(1S)-1-{[(1S)-1-{[4-(hydroxymethyl)-2,5-dimethylphenyl]carbamoyl}ethyl]carbamoyl}-2-methylpropyl]carbamate (480 mg, 1.139 mmol) in DCM (5 mL) at 0° C. The resulting mixture was stirred at 25° C. for 2.5 h. The resulting mixture was concentrated under reduced pressure. To the above mixture was added K₂CO₃ (472.12 mg, 3.417 mmol), MeOH (5 mL), THF (5 mL) and H₂O (5 mL) dropwise at 0° C. The resulting mixture was stirred for additional 2 h at 25° C. The resulting mixture was concentrated under reduced pressure. The residue was purified by silica gel column chromatography, eluted with CH₂Cl₂/MeOH (5:1). This resulted in 500 mg of (2S)-2-amino-N-[(1S)-1-{[4-(hydroxymethyl)-2,5-dimethylphenyl]carbamoyl}ethyl]-3-methylbutanamide as a yellow oil. LC-MS (ESI): 322[M+H]⁺.

### Synthesis of 6-(2,5-dioxo-2,5-dihydro-1H-pyrrol-1-yl)-N-[(1S)-1-{[(1S)-1-{[4-(hydroxymethyl)-2,5-dimethylphenyl]carbamoyl}ethyl]carbamoyl}-2-methylpropyl]hexanamide

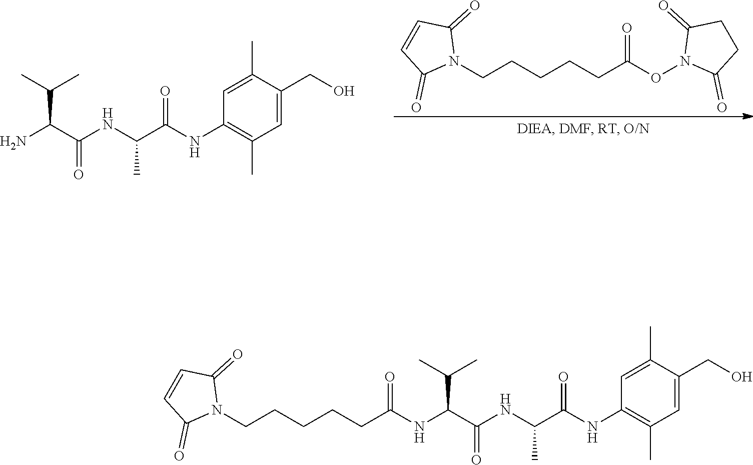 [SMILES: Cc1cc(NC(=O)[C@H](C)NC(=O)[C@@H](N)C(C)C)c(C)cc1CO ; O=C(CCCCCN1C(=O)C=CC1=O)ON1C(=O)CCC1=O ; Cc1cc(NC(=O)[C@H](C)NC(=O)[C@@H](NC(=O)CCCCCN2C(=O)C=CC2=O)C(C)C)c(C)cc1CO]

DIEA (386 mg, 2.986 mmol) was added to a mixture of (2S)-2-amino-N-[(1S)-1-{[4-(hydroxymethyl)-2,5-dimethylphenyl]carbamoyl}ethyl]-3-methylbutanamide (480 mg, 1.493 mmol) and 2,5-dioxopyrrolidin-1-yl 6-(2,5-dioxo-2,5-dihydro-1H-pyrrol-1-yl)hexanoate (460 mg, 1.493 mmol) in DMF (5 mL). The resulting mixture was stirred at 25° C. for 3 h. The resulting mixture was diluted with EtOAc, washed with water, and concentrated. The residue was purified by silica gel column chromatography, eluted with CH₂Cl₂/MeOH (10:1). This resulted in 600 mg of 6-(2,5-dioxo-2,5-dihydro-1H-pyrrol-1-yl)-N-[(1S)-1-{[(1S)-1-{[4-(hydroxymethyl)-2,5-dimethylphenyl]carbamoyl}ethyl]carbamoyl}-2-methylpropyl]hexanamide as a yellow solid. LC-MS (ESI): 515 [M+H]⁺.

### Synthesis of 6-(2,5-dioxo-2,5-dihydro-1H-pyrrol-1-yl)-N-[(1S)-1-{[(1S)-1-{[4-(iodomethyl)-2,5-dimethylphenyl]carbamoyl}ethyl]carbamoyl}-2-methylpropyl]hexanamide

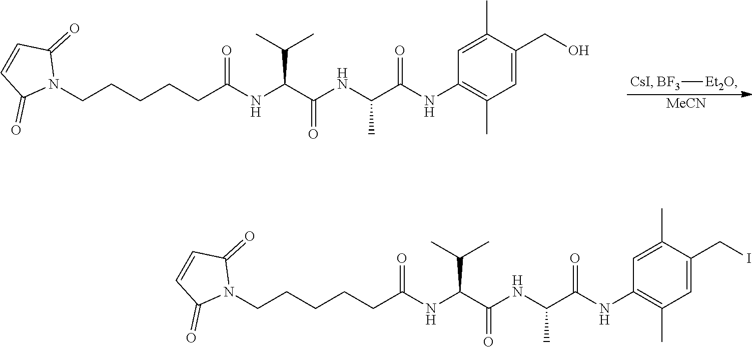

Cesium iodide (76 mg, 0.291 mmol) and BF₃·Et₂O (42 mg, 0.291 mmol) were added to a mixture of 6-(2,5-dioxo-2,5-dihydro-1H-pyrrol-1-yl)-N-[(1S)-1-{[(1S)-1-{[4-(hydroxymethyl)-2,5-dimethylphenyl]carbamoyl}ethyl]carbamoyl}-2-methylpropyl]hexanamide (100 mg, 0.194 mmol) in MeCN (3 mL). The resulting mixture was stirred at 25° C. for 2 h under nitrogen atmosphere. The resulting mixture was diluted with DCM, washed with water, and concentrated. The residue was purified by silica gel column chromatography, eluted with CH₂Cl₂/2-Propanol (10:1). This resulted in 85 mg of 6-(2,5-dioxo-2,5-dihydro-1H-pyrrol-1-yl)-N-[(1S)-1-{[(1S)-1-{[4-(iodomethyl)-2,5-dimethylphenyl]carbamoyl}ethyl]carbamoyl}-2-methylpropyl]hexanamide as a white solid. LC-MS (ESI): 625[M+H]⁺.

### Synthesis of N-[(1S)-1-{[(1S)-1-{[4-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2,5-dimethylphenyl]carbamoyl}ethyl]carbamoyl}-2-methylpropyl]-6-(2,5-dioxo-2,5-dihydro-1H-pyrrol-1-yl)hexanamide (LP52)

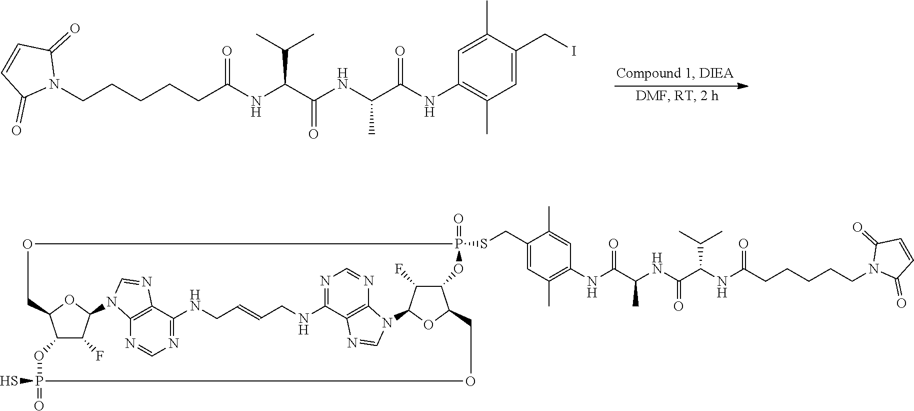

DIEA (43 mg, 0.335 mmol) was added to a mixture of Compound 1 (50 mg, 0.067 mmol) and 6-(2,5-dioxo-2,5-dihydro-1H-pyrrol-1-yl)-N-[(1S)-1-{[(1S)-1-{[4-(iodomethyl)-2,5-dimethylphenyl]carbamoyl}ethyl]carbamoyl}-2-methylpropyl]hexanamide (84 mg, 0.134 mmol) in DMF (2 mL). The resulting mixture was stirred at 25° C. for 1 h. The crude was purified by Prep-HPLC with the following conditions Column: Xbridge Prep Phenyl OBD Column, 19*250 mm, 5 μm; Mobile Phase A: Water(0.1% FA), Mobile Phase B: MeCN; Flow rate: 25 mL/min; Gradient: 30% B to 45% B in 10 min, 45% B; Wavelength: 254 nm; RT1(min): 9. This resulted in 8.7 mg (10.45%) of N-[(1S)-1-{[(1S)-1-{[4-({[(1R,3R,15E,28R,29R,30R,31R,34,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)-2,5-dimethylphenyl]carbamoyl}ethyl]carbamoyl}-2-methylpropyl]-6-(2,5-dioxo-2,5-dihydro-1H-pyrrol-1-yl)hexanamide (LP52) as a white solid.

LC-MS (ESI): 1243.5[M+H]⁺.

¹H NMR (400 MHz, DMSO-d₆) δ 9.15 (s, 1H), 8.66 (s, 1H), 8.39-7.99 (m, 4H), 7.81 (d, J=8.8 Hz, 2H), 7.24-7.06 (m, 2H), 7.00 (s, 3H), 6.88-6.64 (m, 1H), 6.57 (d, J=15.8 Hz, 1H), 6.46-6.32 (m, 1H), 5.79-5.33 (m, 5H), 5.32-4.63 (m, 2H), 4.63-4.54 (m, 2H), 4.52-4.29 (m, 6H), 4.26-4.09 (m, 2H), 4.05-3.81 (m, 3H), 3.42-3.20 (m, 3H), 2.48-2.42 (m, 1H), 2.22-2.02 (m, 5H), 1.99-1.93 (m, 2H), 1.54-1.42 (m, 3H), 1.32-1.11 (m, 6H), 0.82 (t, J=7.7 Hz, 6H).

### 52. Synthesis of LP53

The synthesis of LP53 is shown below:

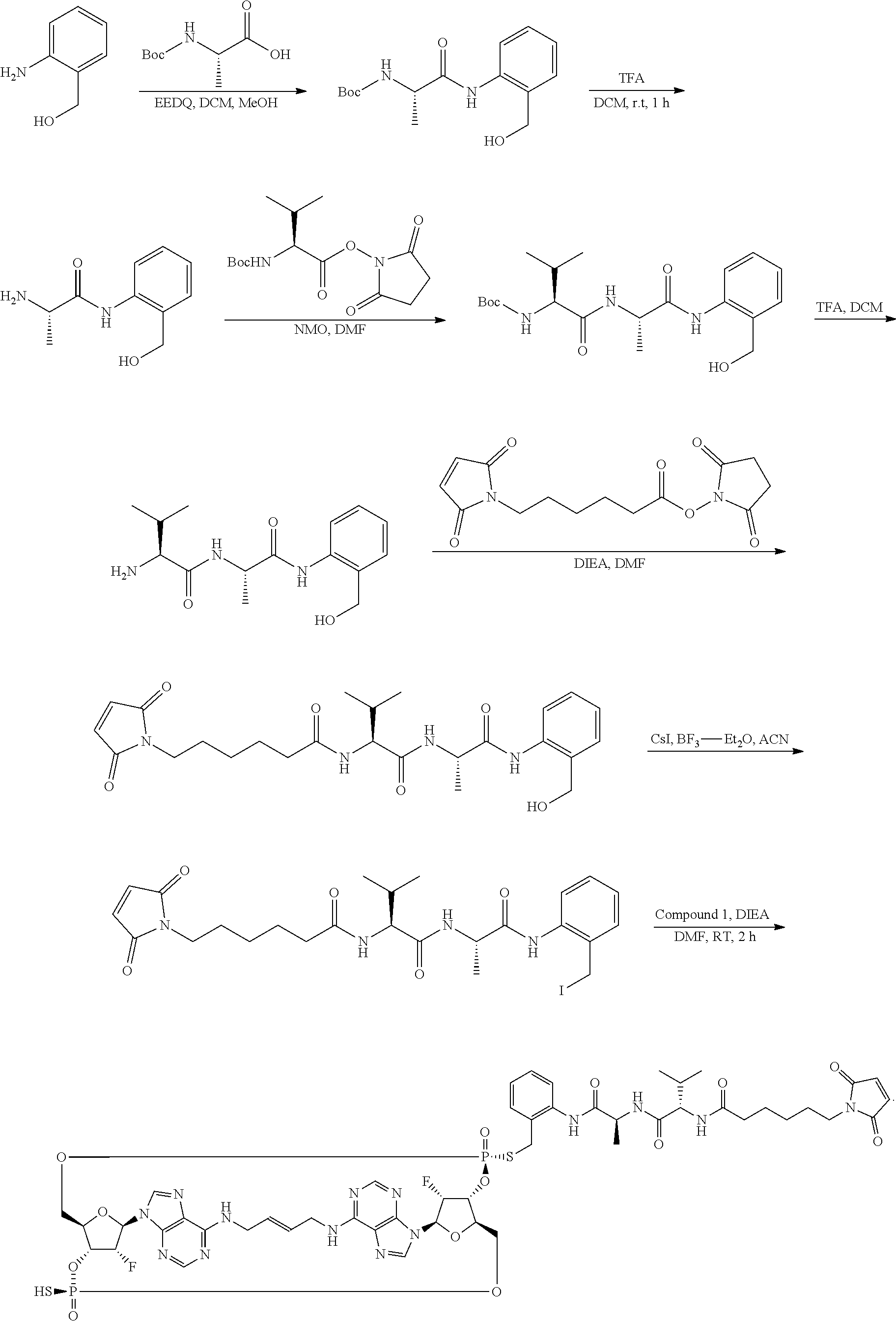

### Synthesis of tert-butyl N-[(1S)-1-{[2-(hydroxymethyl)phenyl]carbamoyl}ethyl]carbamate

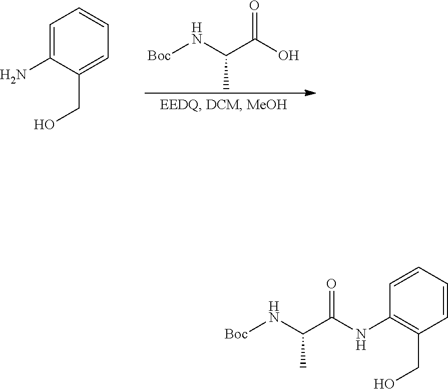 [SMILES: Nc1ccccc1CO ; C[C@H](NC(=O)OC(C)(C)C)C(=O)O ; C[C@H](NC(=O)OC(C)(C)C)C(=O)Nc1ccccc1CO]

Ethyl 2-ethoxy-1,2-dihydroquinoline-1-carboxylate (16.1 g, 64.958 mmol) was added to a mixture of (2-aminophenyl)methanol(4 g, 32.479 mmol) and (2S)-2-[(tert-butoxycarbonyl)amino]propanoic acid (6.2 g, 32.479 mmol) in DCM (24 mL) and MeOH (4 mL). The resulting mixture was stirred at 25° C. for 2.5 h. The resulting mixture was concentrated under reduced pressure. The resulting mixture was diluted with EtOAc, washed with water, and concentrated. The residue was purified by silica gel column chromatography, eluted with PE/EtOAc (1:2). This resulted in 9 g of tert-butyl N-[(1S)-1-{[2-(hydroxymethyl)phenyl]carbamoyl}ethyl]carbamate as a white solid. LC-MS (ESI): 295 [M+H]⁺.

### Synthesis of (2S)-2-amino-N-[2-(hydroxymethyl)phenyl]propanamide

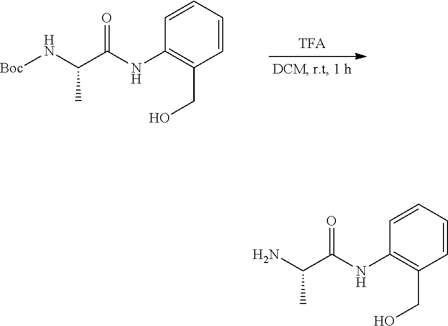 [SMILES: C[C@H](NC(=O)OC(C)(C)C)C(=O)Nc1ccccc1CO ; C[C@H](N)C(=O)Nc1ccccc1CO]

TFA (20 ml) was added to a mixture of tert-butyl N-[(1S)-1-{[2-(hydroxymethyl)phenyl]carbamoyl}ethyl]carbamate (9 g, 30.576 mmol) in DCM (20 mL) at 0° C. The resulting mixture was stirred at 25° C. for 2 h. The resulting mixture was concentrated under reduced pressure. The residue was purified by silica gel column chromatography, eluted with CH₂Cl₂/MeOH (5:1). This resulted in 5 g of (2S)-2-amino-N-[2-(hydroxymethyl)phenyl]propanamide as a yellow solid. LC-MS (ESI): 195 [M+H]⁺.

### Synthesis of tert-butyl N-[(1S)-1-{[(1S)-1-{[2-(hydroxymethyl)phenyl]carbamoyl}ethyl]carbamoyl}-2-methylpropyl]carbamate

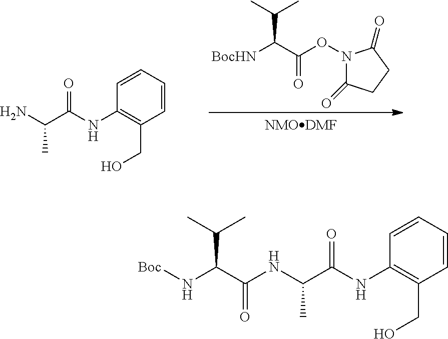 [SMILES: C[C@H](N)C(=O)Nc1ccccc1CO ; CC(C)[C@H](NC(=O)OC(C)(C)C)C(=O)ON1C(=O)CCC1=O ; CC(C)[C@H](NC(=O)OC(C)(C)C)C(=O)N[C@@H](C)C(=O)Nc1ccccc1CO]

NMO (5.3 g, 45.306 mmol) was added to a mixture of (2S)-2-amino-N-[2-(hydroxymethyl)phenyl]propanamide (4.4 g, 22.653 mmol) and 2,5-dioxopyrrolidin-1-yl (2S)-2-[(tert-butoxycarbonyl)amino]-3-methylbutanoate (7.1 g, 22.653 mmol) in DMF (20 mL) The resulting mixture was stirred at 25° C. for 2 h. The resulting mixture was diluted with EtOAc, washed with water, and concentrated. The residue was purified by silica gel column chromatography, eluted with CH₂Cl₂/MeOH (5:1). This resulted in 5.1 g of tert-butyl N-[(1S)-1-{[(1S)-1-{[2-(hydroxymethyl)phenyl]carbamoyl}ethyl]carbamoyl}-2-methylpropyl]carbamate as a yellow solid.

LC-MS (ESI): 394 [M+H]⁺.

### Synthesis of (2S)-2-amino-N-[(1S)-1-{[2-(hydroxymethyl)phenyl]carbamoyl}ethyl]-3-methylbutanamide

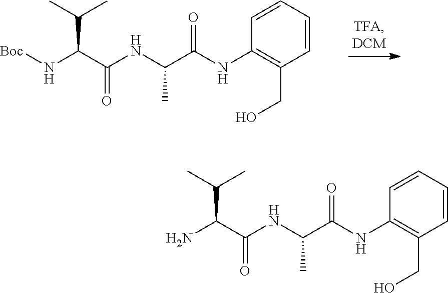 [SMILES: CC(C)[C@H](NC(=O)OC(C)(C)C)C(=O)N[C@@H](C)C(=O)Nc1ccccc1CO ; CC(C)[C@H](N)C(=O)N[C@@H](C)C(=O)Nc1ccccc1CO]

TFA (15 mL) was added to a mixture of tert-butyl N-[(1S)-1-{[(1S)-1-{[2-(hydroxymethyl)phenyl]carbamoyl}ethyl]carbamoyl}-2-methylpropyl]carbamate (5.1 g, 12.961 mmol) in DCM (15 mL) at 0° C. The resulting mixture was stirred at 25° C. for 2.5 h. The resulting mixture was concentrated under reduced pressure. To the above mixture was added K₂CO₃ (5.4 g, 38.883 mmol), MeOH (10 mL), THF (10 mL) and H₂O (10 mL) dropwise at 0° C. The resulting mixture was stirred for additional 2 h at 25° C. The resulting mixture was concentrated under reduced pressure. The residue was purified by silica gel column chromatography, eluted with CH₂Cl₂/MeOH (5:1). This resulted in 2.52 g of (2S)-2-amino-N-[(1S)-1-{[2-(hydroxymethyl)phenyl]carbamoyl}ethyl]-3-methylbutanamide as a white solid. LC-MS (ESI): 294[M+H]⁺.

### Synthesis of 6-(2,5-dioxo-2,5-dihydro-1H-pyrrol-1-yl)-N-[(1S)-1-{[(1S)-1-{[2-(hydroxymethyl)phenyl]carbamoyl}ethyl]carbamoyl}-2-methylpropyl]hexanamide

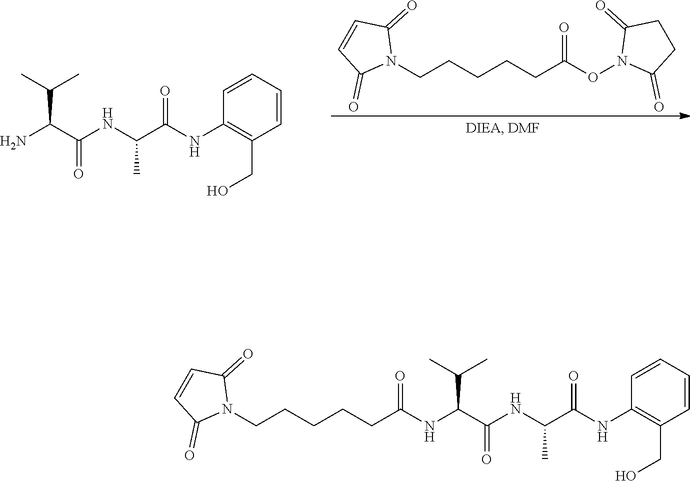 [SMILES: CC(C)[C@H](N)C(=O)N[C@@H](C)C(=O)Nc1ccccc1CO ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccccc1CO]

DIEA (2.2 g, 17.044 mmol) was added to a mixture of (2S)-2-amino-N-[(1S)-1-{[2-(hydroxymethyl)phenyl]carbamoyl}ethyl]-3-methylbutanamide (2.5 g, 8.522 mmol) and 2,5-dioxopyrrolidin-1-yl 6-(2,5-dioxo-2,5-dihydro-1H-pyrrol-1-yl)hexanoate (2.63 g, 8.522 mmol) in DMF (10 mL). The resulting mixture was stirred at 25° C. for 2 h. The resulting mixture was diluted with EtOAc, washed with water, and concentrated. The residue was purified by silica gel column chromatography, eluted with PE/EtOAc (1:1). This resulted in 1.65 g of 6-(2,5-dioxo-2,5-dihydro-1H-pyrrol-1-yl)-N-[(1S)-1-{[(1S)-1-{[2-(hydroxymethyl)phenyl]carbamoyl}ethyl]carbamoyl}-2-methylpropyl]hexanamide as a yellow solid. LC-MS (ESI): 487 [M+H]⁺.

### Synthesis of 6-(2,5-dioxo-2,5-dihydro-1H-pyrrol-1-yl)-N-[(1S)-1-{[(1S)-1-{[2-(iodomethyl)phenyl]carbamoyl}ethyl]carbamoyl}-2-methylpropyl]hexanamide

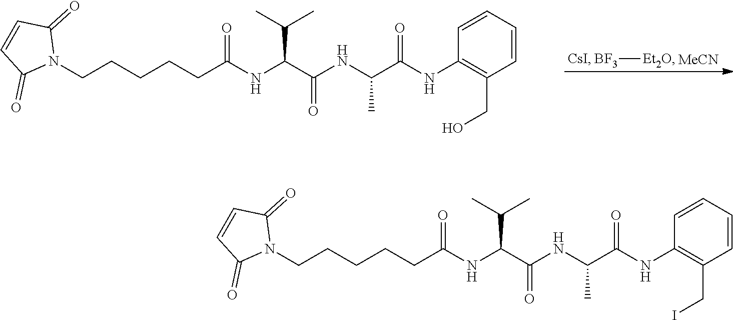

Cesium iodide (400 mg, 1.542 mmol) and BF₃·Et₂O (219 mg, 1.542 mmol) were added to a mixture of 6-(2,5-dioxo-2,5-dihydro-1H-pyrrol-1-yl)-N-[(1S)-1-{[(1S)-1-{[2-(hydroxymethyl)phenyl]carbamoyl}ethyl]carbamoyl}-2-methylpropyl]hexanamide (500 mg, 1.028 mmol) in MeCN (5 mL). The resulting mixture was stirred at 25° C. for 2 h under nitrogen atmosphere. The resulting mixture was diluted with DCM, washed with water, and concentrated. The residue was purified by silica gel column chromatography, eluted with CH₂Cl₂/2-Propanol (7:1). This resulted in 300 mg of 6-(2,5-dioxo-2,5-dihydro-1H-pyrrol-1-yl)-N-[(1S)-1-{[(1S)-1-{[2-(iodomethyl)phenyl]carbamoyl}ethyl]carbamoyl}-2-methylpropyl]hexanamide as a white solid. LC-MS (ESI): 597[M+H]⁺.

### Synthesis of N-[(1S)-1-{[(1S)-1-{[2-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP53)

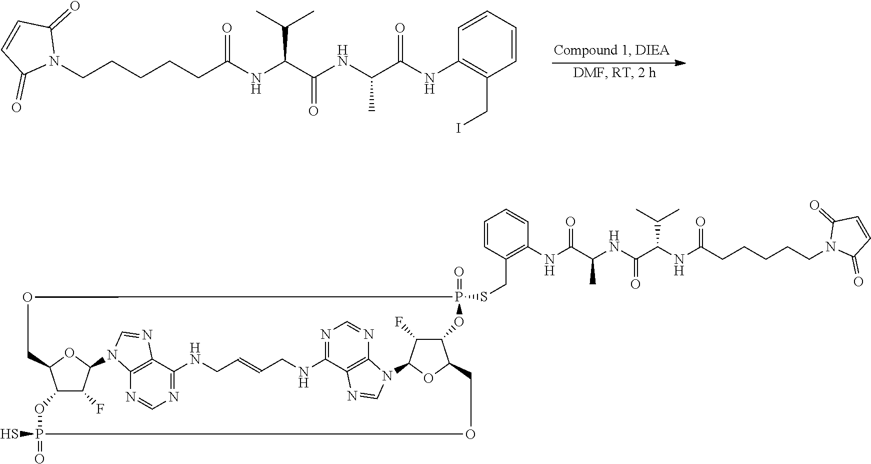

DIEA (43 mg, 0.335 mmol) were added to a mixture of Compound 1 (50 mg, 0.067 mmol) and 6-(2,5-dioxo-2,5-dihydro-1H-pyrrol-1-yl)-N-[(1S)-1-{[(1S)-1-{[2-(iodomethyl)phenyl]carbamoyl}ethyl]carbamoyl}-2-methylpropyl]hexanamide (80 mg, 0.134 mmol) in DMF (2 mL). The resulting mixture was stirred at 25° C. for 1 h. The crude was purified by Prep-HPLC with the following conditions; Column: XSelect CSH Prep C18 OBD Column, 19*250 mm, 5 μm; Mobile Phase A: Water(10 mmol/L NH₄HCO₃), Mobile Phase B: MeCN; Flow rate: 25 mL/min; Gradient: 30% B to 30% B in 13 min, 30% B; Wavelength: 254 nm; RT1(min): 11.2. This resulted in 12.7 mg of N-[(1S)-1-{[(1S)-1-{[2-({[(1R,3R,15E,28R,29R,30R,31R,34S,36R,39R,41R)-29,41-difluoro-34,39-dioxo-39-sulfanyl-2,33,35,38,40,42-hexaoxa-4,6,9,11,13,18,20,22,25,27-decaaza-34lambda5,39lambda5-diphosphaoctacyclo[28.6.4.1^{3,36}.1^{28,31}.0^{4,8}.0^{7,12}.0^{19,24}.0^{23,27}]dotetraconta-5,7,9,11,15,19,21,23,25-nonaen-34-yl]sulfanyl}methyl)phenyl]carbamoyl}ethyl]carbamoyl}-2-methylpropyl]-6-(2,5-dioxo-2,5-dihydro-1H-pyrrol-1-yl)hexanamide (LP53) as a white solid. LC-MS (ESI): 1215.30[M+H]⁺.

### Example 2. Humanization of J591

### 1 Methods

### 1.1 in Silico Modeling

J591 was modeled in Schrodinger's BioLuminate using the antibody prediction tool. J591 was aligned to the closest human germline, IGHV1-2*06 and IGKV1-13*02. Analyzing the prevalence of these subfamilies in the human population, these subfamilies are not highly represented or paired with each other. See, e.g., Tiller et al. (2013) mAbs 5:3, 445-470. Therefore, the very similar subfamilies IGHV1-69-2*01 and IGKV1-39*01 were used.

The variable domains of the mouse and CDR-grafted sequences were used to generate in silico structural models. Models were generated using Schrodinger's BioLuminate software, following the homology domain protocol.

### 1.2 Gene Synthesis and Cloning

### 1.2.1 InFusion Cloning

Humanized heavy and light variable domains were codon-optimized for expression in HEK293 cells and were synthesized by Thermo Fisher Scientific. The variable domains were synthesized with a Kozak translation initiation sequence and an Ig secretion leader sequence and included 15 base-pairs at the 5′ and 3′ ends homologous to the cloning site within the subcloning vector. PCR fragments synthesized by GeneArt were subcloned into an expression plasmid containing a human gamma or kappa constant region using an InFusion HD cloning kit (Clontech). All clones were sequenced to confirm the presence and fidelity of the inserts.

### 1.2.2 Site-Directed Mutagenesis

Mutations were generated using Agilent's QuikChange Lightning according to the manufacturer's protocol. The desired mutations were confirmed by DNA sequencing.

### 1.3 Cell Culture

### 1.3.1 Transfection and Stable Cell Line Generation

For each milliliter of cells to be transfected with ExpiFectamine (ThermoFisher), 333.3 ng HC plasmid and 333.3 ng LC plasmid was incubated for 5-10 min in 50 μL Opti-MEM (ThermoFisher). Likewise, 2.67 μL ExpiFectamine was incubated in 50 μL Opti-MEM. The ExpiFectamine solution was added to the DNA mixture and incubated for 20-30 min at room temperature. The DNA:ExpiFectamine mixture was added to the cells while swirling and incubated at 37° C., 8% CO₂, shaking at 125 rpm. The following day, 5 μL of enhancer 1 and 50 μL of enhancer 2 per mL of cells were added to the transfection with continued incubation for another 7-10 days.

Antibody-expressing stable pools were selected by adding 1 mL of transfectants to 14 mL DMEM with 10% fetal bovine serum in a T75 flask with 5 μg/mL blasticidin (ThermoFisher) and 400 μg/mL zeocin (Invivogen) one to three days after transfection. After drug-resistant cells grew to confluency, the medium was replaced with FreeStyle 293 expression medium for 24 to 48 h. Cells were physically dislodged by tapping the flask (trypsinization resulted in low viability, data not shown) and were then seeded at 6×10⁵ cells/mL in 30 mL FreeStyle 293 expression medium in a 125-mL shake flask. Cultures were incubated at 37° C. in 8% CO₂ with shaking at 125 rpm.

### 1.3.2 MAb and Fab Production

Stably-transfected cell line pools were seeded at 0.6×10⁶ to 1×10⁶ cells/mL in FreeStyle 293 expression medium. Cells were incubated at 37° C., 8% CO₂, shaking at 125 rpm. Two days after the culture reached a density of 1×10⁶ cells/mL, cultures were fed with final concentrations of 10 g/L Select Soytone (BD Biosciences), 5 mM valeric acid (Sigma Aldrich), and 1:100 CD Lipid Concentrate (ThermoFisher). When the cell viability was less than 50% (7-10 days), the cultures were centrifuged for 30 min at 8000 rpm in a Beckman JLA8.1000 rotor. The supernatant was then filtered through a 0.2 μm PES filter and stored at 4° C. or −20° C. until purification.

### 1.4 MAb and Fab Purification

MAbs were purified using one of two methods. For mAb and Fab supernatants less than 10 mL, affinity chromatography was performed using a batch purification method with protein A resin or anti-kappa resin, respectively. MAb and Fab supernatants greater than 25 mL were purified using pre-packed protein A or anti-kappa columns, respectively.

### 1.4.1 Batch Purification

Prosep-vA High Capacity Protein A resin (Millipore) or CaptureSelect™ KappaSelect LC-kappa resin (ThermoFisher) was equilibrated with DPBS, and 100 μL were added to 3 to 6 mL of sample. Following incubation at 4° C. for 1 hour to overnight, the resin was washed three times with 1 mL DPBS and centrifuged at 15,000×g for 30 s. The sample was eluted from the resin by addition of 400 μL 0.1 M Glycine, pH 2.9 followed by centrifugation at 15,000×g for 30 s. The sample was neutralized with 40 μL of 1 M Tris, pH 8.0. The buffer was exchanged using 0.5 mL Amicon Ultra, 10 k cutoff filters (Millipore) by concentrating the sample to ~100 μL by centrifugation at 15,000×g for 3 to 5 minutes. The concentrated sample was diluted in 400 μL DPBS, followed by centrifugation. The process was repeated a total of four times.

### 1.4.2 Column Purification

A protein A or HiTrap KappaSelect column (GE Healthcare) was equilibrated with 10 column volumes (CV) of 20 mM sodium phosphate, 10 mM EDTA, pH 7.2. The sample was then loaded, followed by washing unbound material with 10 CV of equilibration buffer. The sample was eluted using 5 CV of 0.1 M Glycine, pH 2.9. The fractions containing the mAb were pooled and dialyzed in DPBS using a MWCO 20K Slide-A-Lyzer (ThermoFisher).

### 1.5 ELISA Screening

ELISA plates (384-well high-binding Greiner #781061) were coated with 1 μg/mL of antigen in 50 mM carbonate-bicarbonate, pH 9.4 (Pierce #28382), and incubated overnight at 4° C. Plates were washed three times with wash buffer using BioTek ELX405 plate washers. Wash buffer was made from PBS (Corning #46-013-CM) and 0.05% Tween20 (SeraCare #5460-0020). Plates were blocked with Assay Buffer made from PBS with 0.05% Tween20 and 1% BSA (Sigma A7906) and incubated overnight at 4° C. Samples were added after aspirating the plates, then incubated overnight at 4° C. Plates were washed three times, then HRP-conjugated Goat anti-Rabbit IgG H+L antibody (Jackson ImmunoResearch #111-035-144) at a 1:10,000 dilution in Assay Buffer was added, and incubated for 1 hour at room temperature. Plates were washed three times and TMB substrate was added (SeraCare #5120-0078) and incubated for 20 minutes at room temperature. Plates were read at 370 nm on a BMG CLARIOstar plate reader.

### 1.6 BIAcore Methods For Binding Analysis

### Method Overview

The affinity of anti-PSMA antibody samples to human PSMA was determined by surface plasmon resonance using a capture assay format whereby antibody ligand was first captured by immobilized anti-human Fc on a BIAcore CM5 chip. The affinity of antibody samples was determined using a multi-cycle binding assay format with injection of a concentration series of human PSMA. Data fitting was performed using a 1:1 Langmuir model using BIAEvaluations.

### Anti-Human Antibody Chip Immobilization

A standard coupling protocol was employed to immobilize the Fc-specific IgG via exposed primary amines. The stock anti-Fc IgG solution (Cytiva, BR100839) was diluted to 25 μg/mL with 10 mM sodium acetate pH 5.0 (Cytiva, BR1008390). The immobilization was performed at 25° C. using 1×HBS-P+ (Cytiva, BR100671) as the running buffer. The CM-dextran surfaces of all four flow cells on a CM5 biosensor chip (Cytiva, 29149603) were activated by a 7 min injection (10 μL/min) of freshly prepared 1:1 NHS:EDC (Cytiva, BR100050). Then the 25 μg/mL anti-Fc IgG solution was injected for 6 min at a flow of 5 μl/min. This coupling was followed by a 7 min injection (10 μl/min) of 1 M ethanolamine (Cytiva, BR100050), which deactivated residual reactive sites. Typically, 6,000 RU of anti-Fc IgG were coupled using this method.

### Further Method Details

The screening analysis was performed at 25° C. and a data collection rate of 10 Hz on a BIAcore T-200 instrument (Cytiva). Ligand and analyte stock solutions were prepared in running buffer (0.2% BSA in 1×HBS-P+, (BSA from VWR, 9048-46-8)) and centrifuged 18,000× rcf for 5 minutes at ambient temperature.

Ligand solutions were subsequently diluted to final concentrations of 5 μg/mL, and human PSMA analytes (R and D Systems) were diluted to final concentrations of 50, 12.5, 3.125, 0.781, 0.195, 0.049, 0.012, and 0 nM. Chip conditioning was repeated 5 times where 5 μg/mL of anti-PSMA antibody was injected for 1 min (5 μL/min) over flow cells 2, 3, 4. Surfaces were regenerated with 3 M MgCl₂ (Cytiva, BR100839) for 30 sec (30 μL/min) followed by 180 sec stabilization. To assess kinetic binding, flow cell 1 served as the reference control, and ligand solutions were injected over flow cells 2, 3, 4 for 1 min (5 μL/min). Human PSMA samples were injected in random order at a flow rate of 10 μl/min over the four surfaces. Buffer injections identical to the antigen injections were randomly interspersed for the purpose of double referencing. Association and dissociation phases were monitored for 5 min and 10 min, respectively. Surfaces were regenerated as mentioned in the conditioning step. To determine the kinetic parameters of the interactions, each data set was double-referenced and fit globally to a 1:1 interaction model (Langmuir model using BIAEvaluations).

### 1.7 Differential Scanning Calorimetry (DSC) Analysis

PEAQ Differential Scanning Calorimeter (Malvern Instruments, PEAQ-DSC, s/n MAL1223867 with MicroCal PEAQ-DSC software v.1.53) was used to decipher and compare the higher order structure and thermal stability of various F(ab′)₂ fragments and controls. Samples were allowed to acclimate to ambient temperature for 30 minutes, followed by vortexing. The sample (0.325 mL, 0.85 mg/ml) was added to appropriate wells of an assay plate (Microliter Analytical Supply, 96 well, 500 μL, round well and bottom, cat #07-2100; Sun Suri plate cover cat #300-005). 0.325 mL of 20% Contrad solution and 0.325 mL water was added to the appropriate wells of the assay plate. Sealed plates were placed into auto-sampler maintaining a 4° C. temperature.

The run was programmed and initiated using the following assay parameters:

### DSC Controls:

Start Temperature=20° C.
Final Temperature=100° C.
Scan Rate=60° C./hr
Pre-scan Thermostat=10 Minutes
Post-scan Thermostat=0 Minutes
Feedback Mode/Gain=None

### Sample Parameters:

Well Plate Volume=0.325 mL
Number of Purge Refills=5
Sample Concentration=0.85 mg/ml
Molecular Weight (Daltons)=150,000
Clean Setting=SCAN
Solvent Reservoir=Number 1, 20% Contrad
Rinse Volume=10 mL
Clean in-line with Contrad/Contrad at 20-70° C., 60° C./Hour followed by two Buffer/Buffer injections

### Sample Analysis Parameters:

Y-Axis Scale Units=mCal/Minute
Subtract Buffer
Baseline Fit=Spline
Fit=Non-Two State Fitting Model
Iterate=Yes

### 2 Results

### 2.1 Humanization and Affinity Characterization

In general, the humanizing process involves matching the closest human germline sequence to the animal-derived antibody sequence, then grafting the CDR regions onto the human germline framework. Non-human residues may elicit an immunogenic response resulting in an anti-drug antibody response in patients, thereby neutralizing the therapeutic effect of the antibody. The mouse-derived J591 sequence was used to search for the closest human germline sequence using IGBLAST (FIG. 1, https://www.ncbi.nlm.nih.gov/igblast/). The human VH germline family IGVH1-69-2*01 and the human VL germline IGKV1-39*01 were chosen as the germline frameworks. Humanized variants were generated based on the linear location of the CDRs (defined by both Kabat and IMGT). The J591 CDRs were grafted onto these human frameworks whereby the mouse-derived CDRs replaced the human CDRs. Since there are several definitions of CDR lengths, the CDRs sequences grafted encompassed both IMGT and Kabat definitions (Vhzu1 and Vkzu1). This increased the likelihood that the entire paratope was grafted onto the human framework. Additional mouse or human residues were substituted throughout the variable sequences to increase the humanness without affecting the affinity to PSMA.

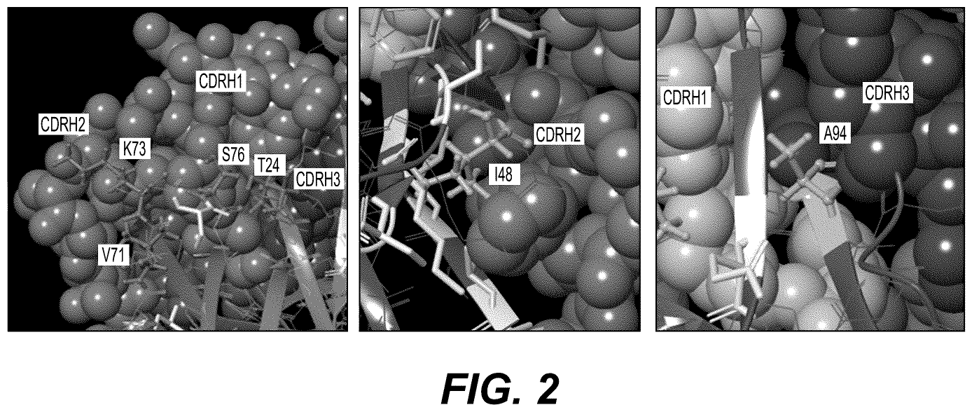

An in silico model was generated of J591, and framework residues adjacent to the CDRs differing between the mouse and Vhzu1 and Vkzu1 humanized sequences were identified (FIG. 2). Subsequent humanized Vh variants 2 to 10 were generated by added mouse residues throughout the framework. No additional mouse Vk residues were identified to have a potential impact on antigen binding.

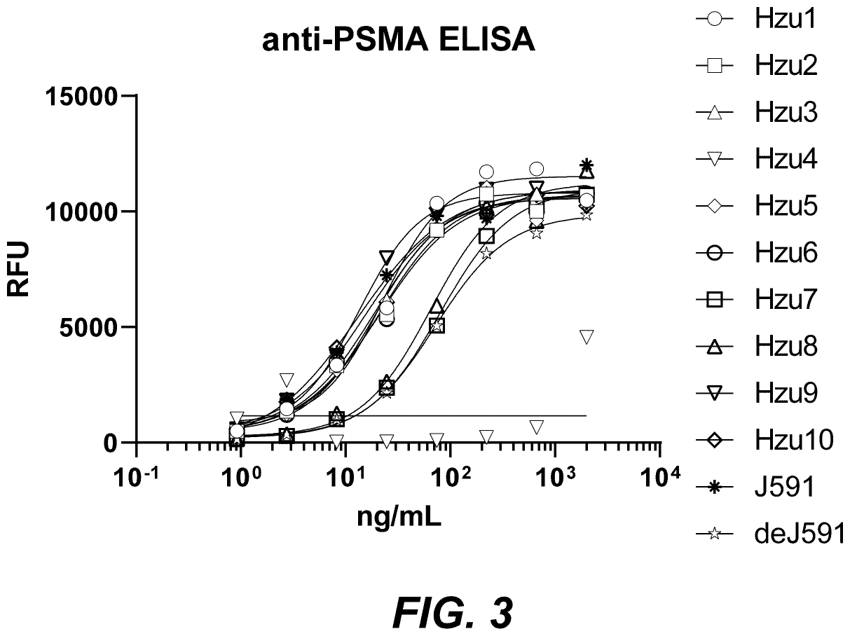

Humanized HCs 1 through 10 were paired with humanized LCzu1 and tested for PSMA binding by ELISA (FIG. 3). PSMA was coated on plates, blocked with a blocking solution, then incubated with various concentrations of antibody. After washing the plates to remove unbound antibody, an HRP-conjugated detection antibody was added. After the removal of unbound detection antibody, TMB was added to the plate. The reaction between HRP and TMB was stopped with H₂SO₄ and the plates were analyzed. All HC variants except zu4 bound to PSMA similar to or better than J591. The HCzu4-LCzu1 antibody expressed poorly, and it is likely that the lack of binding was due to poor protein quantity rather than the HC sequence.

The KD of the antibody-PSMA interaction was analyzed by SPR. The KD for each antibody was determined, and there was little difference (within 2.5-fold) in the KD of each humanized variant, deJ591, and J591 (Table 17). The kinetics of binding between J591 antibody variants and PSMA was analyzed by SPR using a BIAcore T-200 instrument.

TABLE 17
All humanized HCs bind PSMA at similar affinity to J591 and deJ591

| | kₐ | k_{d} | K_{D} | Notes |
|---|---|---|---|---|
| HCzu1LCzu1 | 3.24E+04 | 6.88E−05 | 2.12E−09 | |
| HCzu2LCzu1 | 2.90E+04 | 6.28E−05 | 2.17E−09 | |
| HCzu3LCzu1 | 3.45E+04 | 7.72E−05 | 2.24E−09 | |
| HCzu4LCzu1 | 6.95E+04 | 2.39E−04 | 3.45E−09 | poor antibody capture |
| HCzu5LCzu1 | 3.85E+04 | 1.19E−04 | 3.10E−09 | |
| HCzu6LCzu1 | 3.94E+04 | 1.07E−04 | 2.72E−09 | |
| HCzu7LCzu1 | 5.82E+04 | 1.00E−04 | 1.72E−09 | |
| HCzu8LCzu1 | 6.14E+04 | 9.38E−05 | 1.53E−09 | |
| HCzu9LCzu1 | 7.33E+04 | 1.01E−04 | 1.38E−09 | |
| HCzu10LCzu1 | 5.99E+04 | 1.05E−04 | 1.74E−09 | |
| J591 | 1.03E+05 | 1.27E−04 | 1.22E−09 | |
| de J591 | 7.81E+04 | 1.43E−04 | 1.82E−09 | |

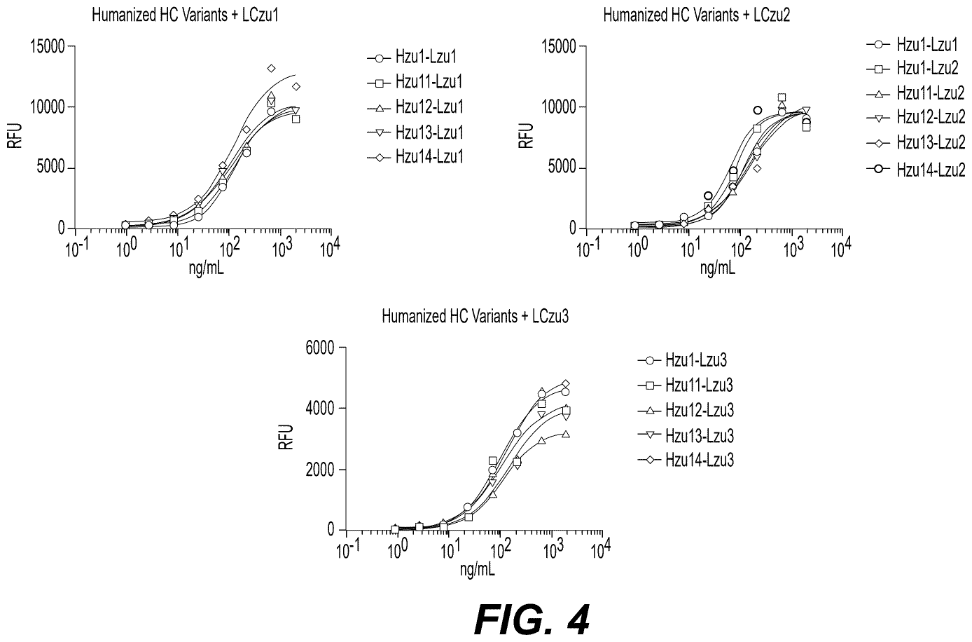

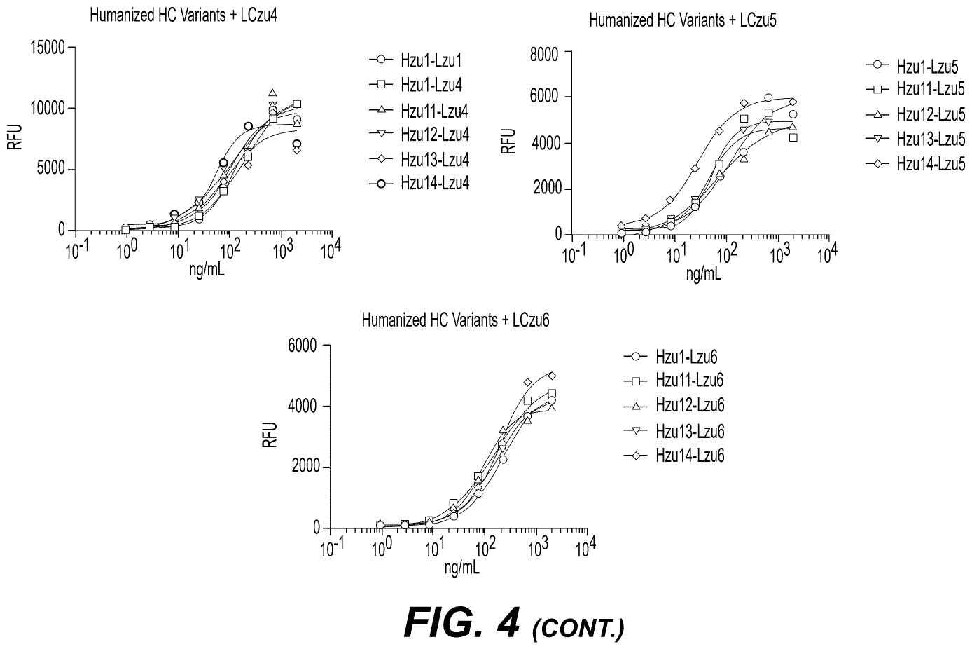

The most humanized HC-LC combination in the first set was HCzu1-LCzu1, demonstrating that no additional mouse residues were required in the human framework to support antigen binding. The sequences were then super-humanized by adding back more human residues at Kabat-defined positions 60 and 61 in CDRH2, residues 24, 33, and 34 in CDRL1, and residue 56 in CDRL2 (FIG. 1). Each HC was paired with each LC and PSMA binding was analyzed by ELISA. Comparing individual HCs paired with the various LCs, the variations in the HC had little effect on antigen binding (FIG. 4). The LCzu3 and LCzu6 pairings tended to show weaker binding than the other super-humanized variants. Both LC variants contain Leu and Asn residues at positions 33 and 34, respectively, suggesting the mouse residues in this region improve PSMA binding. Several of these antibody variants were chosen for further characterization.

HCzu1, HCzu2, HCzu3, and HCzu14 were each paired with LCzu1. The affinity of these antibodies for PSMA was analyzed by SPR. There was very little difference in KD between the humanized HC variants paired with LCzu1 (Table 18). HCzu14 is the most human sequence and was chosen as the lead HC variant. HCzu14 was paired with each humanized LC variant and the affinity for PSMA was measured by SPR (Table 19). HCzu14 paired with LCzu1, 2, 4, and 5 retained affinity for PSMA similar to J591. As was seen with the ELISA screening, pairing with LCzu3 and LCzu6 resulted in a reduction in affinity, again showing the role of CDRL2 residues 33 and 34.

TABLE 18
The most-humanized variant Hczu14-LCzu5
retains PSMA affinity similar to J591

| | kₐ | k_{d} | K_{D} |
|---|---|---|---|
| J591-HCzu1LCzu1 | 5.29E+04 | 1.05E−04 | 1.98E−09 |
| J591-HCzu2LCzu1 | 2.84E+04 | 7.71E−05 | 2.71E−09 |
| J591-HCzu3LCzu1 | 3.89E+04 | 8.45E−05 | 2.17E−09 |
| J591-HCzu14LCzu1 | 3.50E+04 | 1.42E−04 | 4.07E−09 |
| J591-HCzu14LCzu5 | 4.14E+04 | 1.30E−04 | 3.13E−09 |
| J591 | 1.06E+05 | 1.21E−04 | 1.14E−09 |

TABLE 19
The most-humanized variant Hczu14-LCzu5
retains PSMA affinity similar to J591

| Ligand | kₐ | k_{d} | K_{D} |
|---|---|---|---|
| J591 | 5.69E+04 | 6.56E−05 | 1.14E−09 |
| Hzu14-Lzu1 | 3.79E+04 | 5.06E−05 | 1.34E−09 |
| Hzu14-Lzu2 | 2.82E+04 | 5.84E−05 | 2.07E−09 |
| Hzu14-Lzu3 | 2.62E+04 | 9.01E−05 | 3.44E−09 |
| Hzu14-Lzu4 | 3.05E+04 | 5.19E−05 | 1.70E−09 |
| Hzu14-Lzu5 | 2.73E+04 | 5.06E−05 | 1.85E−09 |
| Hzu14-Lzu6 | 2.15E+04 | 1.34E−04 | 6.23E−09 |

### 2.2 Thermal Stability of J591 and Humanized Variants

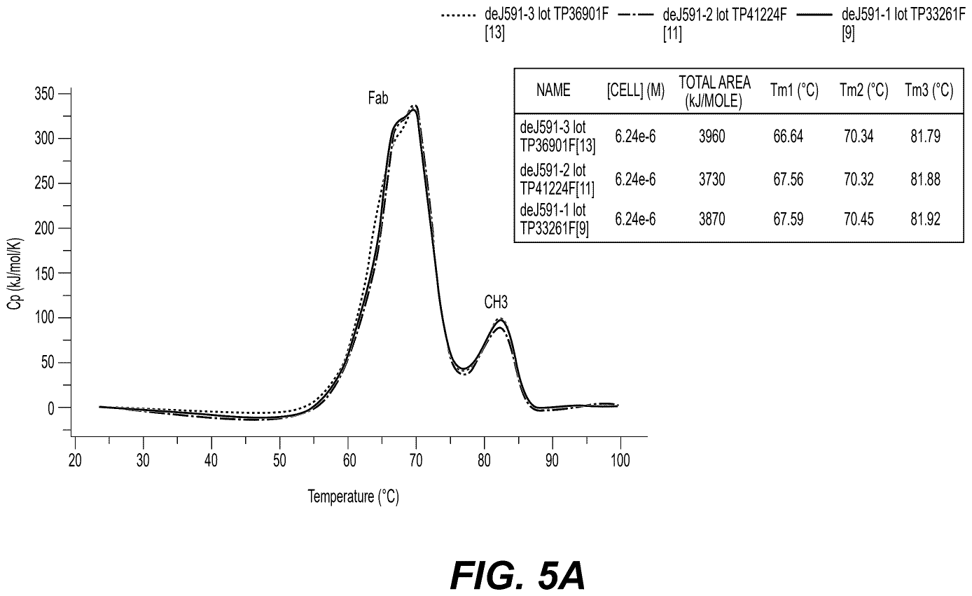

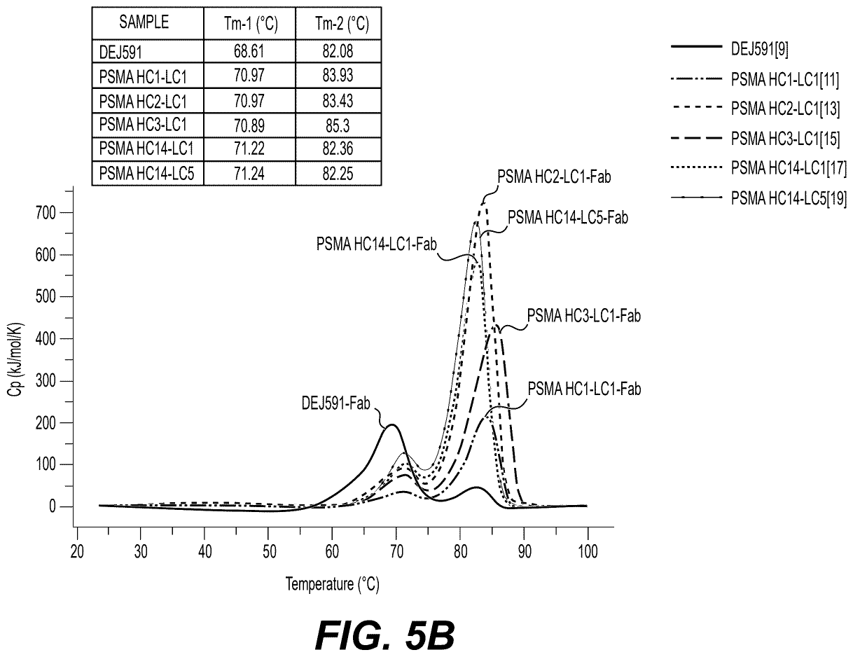

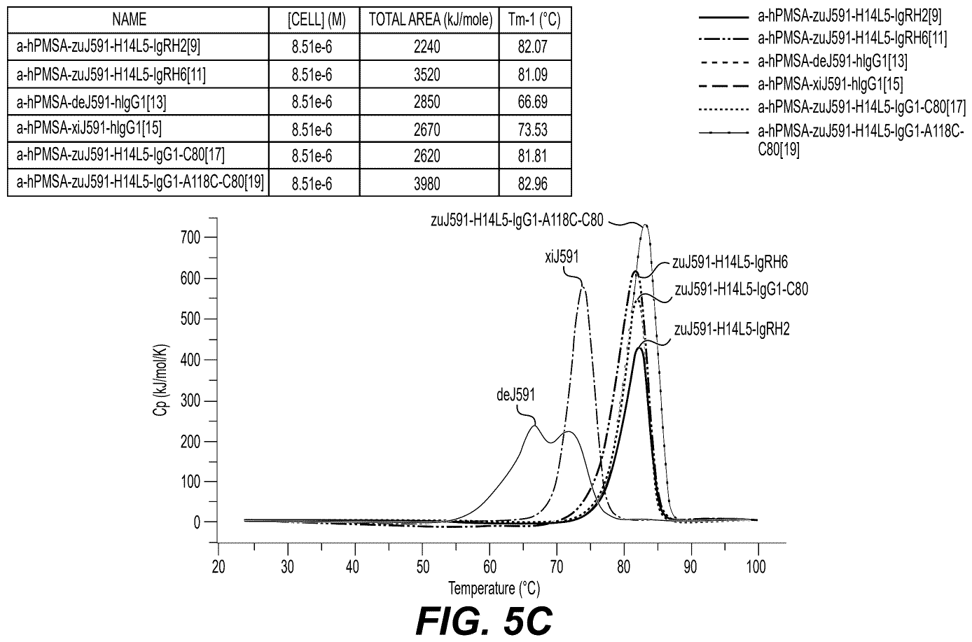

The thermal stability of the humanized antibodies was analyzed by differential scanning calorimetry. The transition midpoints (Tm) of the Fab domains were first analyzed in the context of the full antibody. Three lots of deJ591 were analyzed, and the Fab showed two overlapping peaks with average Tms at 67.3 and 70.37 (FIG. 5A). This profile is very unusual for an antibody. Fabs typically show a single peak with a Tm in the mid-70s to mid-80s ° C. These data suggest great instability in the deJ591 Fab region. The humanized variants HCzu1-LCzu1, HCzu2-LCzu1, HCzu3-LCzu1, HCzu14-LCzu1, and HCzu14-LCzu5 were analyzed by DSC and compared to deJ591 (FIG. 5B). All humanized variants showed a substantial improvement in antibody stability. LCzu1 and LCzu5 showed no difference on antibody stability as seen when comparing HCzu14-LCzu1 and HCzu14-LCzu5. There were minor differences in thermal stability among the HC variants. HCzu3 was the most stable variant with a Tm of 85.3° C. HCzu1 and HCzu2 were very similar with Tm of 83.93° C. and 83.43° C., respectively. HCzu14 showed the lowest Tm at 82.36° C. and 82.25° C. when paired with LCzu1 and LCzu4, respectively. These data suggest that Ile 48, Asn 60, and Gln 61 (defined by Kabat) contribute to Fab stability to some degree. Given that 1) the difference in Tm between HCzu3 and HCzu14 is only 3° C., 2) the Tm of HCzu14-LCzu5 is high, and 3) HCzu14-LCzu5 is the most humanized sequence, the humanized variant HCzu14-LCzu5 was chosen for further development.

The HCzu14-LCzu5-IgG1 antibody was modified to include site-specific conjugation residues. Two acyl acceptor sites for transglutaminase-mediation conjugation and two unpaired, maleimide-reactive cysteines were incorporated into the IgG1 backbone and the stability of the Fabs was analyzed. IgRH2 contains a T135K mutation in CH1 used to generate DAR 2 conjugates. IgRH6 contains T135K and L193K mutations in CH1 to generate DAR 4 conjugates. IgG1-C80 mutates Pro 80 in VK to a Cys to generate DAR 2 conjugates. IgG1-A118C-C80 mutates both Pro 80 to Cys in VK and Ala 118 to Cys in CH1 to generate DAR 4 conjugates. All mutations are within the Fab fragment, and the Tm of each Fab fragment was analyzed. After enzymatic digestion of the full antibody to generate Fab′2 and Fc fragments, the Fc was removed, and the Fab′2 was analyzed by DSC. As was seen in the context of full IgG1 (FIG. 5B), the deJ591 Fab showed two peaks with low Tms (FIG. 5C). The Tm of J591 Fab was a single peak, but significantly (~8° C.) lower than the HCzu14-LCzu5 Fab fragments. The site-specific conjugation sites had little effect on the thermal stability, though IgG1-C80-A118C showed a slightly higher Tm than the other three.

### Example 3. In silica Immunogenicity Prediction ofJ591 and Humanized Variants

### 1 Methods For in Silico Immunogenicity Prediction of J591 and Humanized Variants

Abzena's iTope-AI is an in silico immunogenicity risk assessment platform using a machine learning prediction algorithm for predicting overlapping 9mer peptides binding to 46 isotypes of HLA-DR, DP and DQ isotypes representing the most common HLA alleles. Peptide sequences fully homologous to the human proteome are typically excluded from the analysis. An overall risk score, or Position Risk Score, is calculated by giving individual peptides a binding score from 0 to 3 for each HLA allotype with the scores being added together for all HLA-DR, DP, and DQ alleles. The Position Risk Score of a peptide with a score of 1-2, 3-5, or 6+ is considered a weak, medium or strong promiscuous MHC class II binder, respectively. The entire protein sequence is assigned a Total Score by calculating the sum of the Position Risk Scores for all individual peptides. Peptides with a Position Risk Score are cross-referenced against Abzena's TCED™ database, a repository of >10,000 peptides that stimulated T cell responses in Abzena's ex vivo EpiScreen™ studies. Abzena claims “the algorithm was able to accurately predict 95% of peptide binding core motifs previously identified by 3D structure from X-ray crystallography.” The potential immunogenicity of overlapping 9mer sequences from chimeric mouse J591Vh-hulgG1, mouse J591VK-hukappa, deJ591-hulgG1, deJ591-hukappa, zuJ591-H14-hulgG1, and zuJ591-L5-hukappa were analyzed by Abzena using iTope-AI.

### 2 Results for in Silico Immunogenicity Prediction of J591 and Humanized Variants

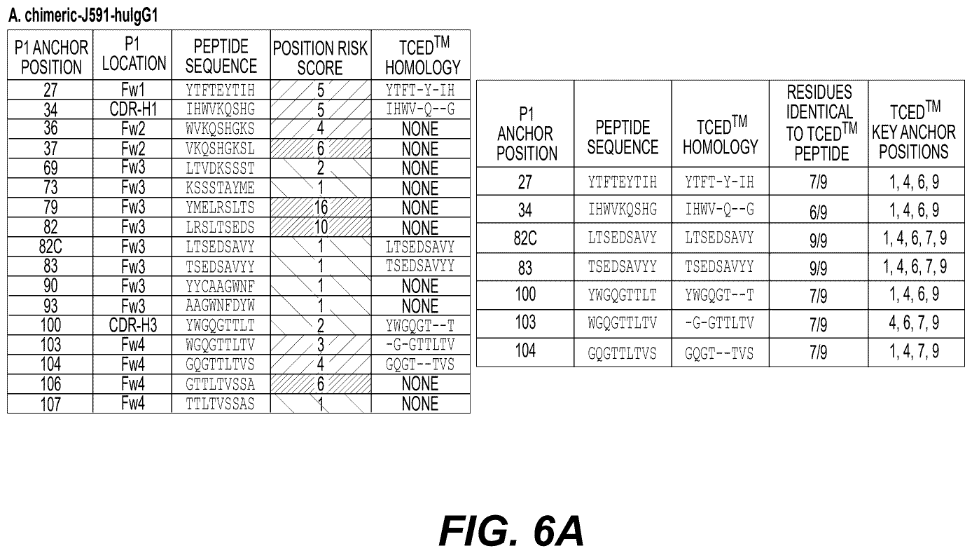

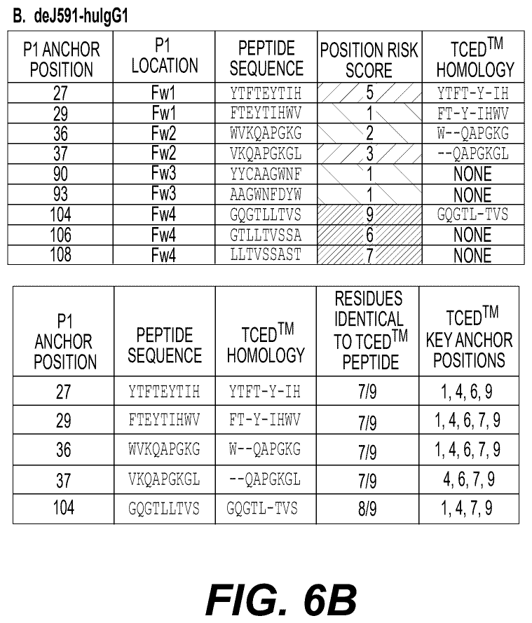

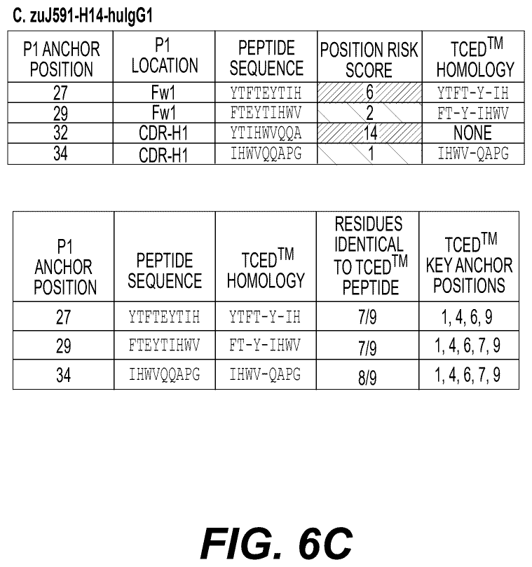

Chimeric mouse J591Vh-hulgG1 contains 17 weak, medium, and strong affinity, non-germline 9mer peptides yielding a Total Score of 69 and a Hotspot Max (i.e., the value of the highest scoring epitope) of 16 (FIG. 6). Seven of these peptides were partial matches to previously identified T cell epitopes. Potentially immunogenic peptides spanned CDRH1, CDRH2, and CDRH3, with a majority overlapping the C-terminal half of the Vh region from FWRH3 through FWRH4. The “deimmunized” deJ591-hulgG1 removes 8 of these peptides and contains 9 weak, medium and strong affinity, non-germline peptides yielding a Total Score of 35 and a Hotspot Max of 9. Five of the peptides were partial matches to previously identified T cell epitopes. These peptides covered CDRH1-FWRH2 and CDRH3-FWRH4. The humanization of zuJ591-H14-hulgG1 further reduced the number of potentially immunogenic peptides to 4 weak and strong affinity, non-germline peptides yielding a Total Score of 23 and a Hotspot Max of 14. Three partial matches to previously identified T cell epitopes were identified. The peptides overlapped with CDRH1-FWRH2.

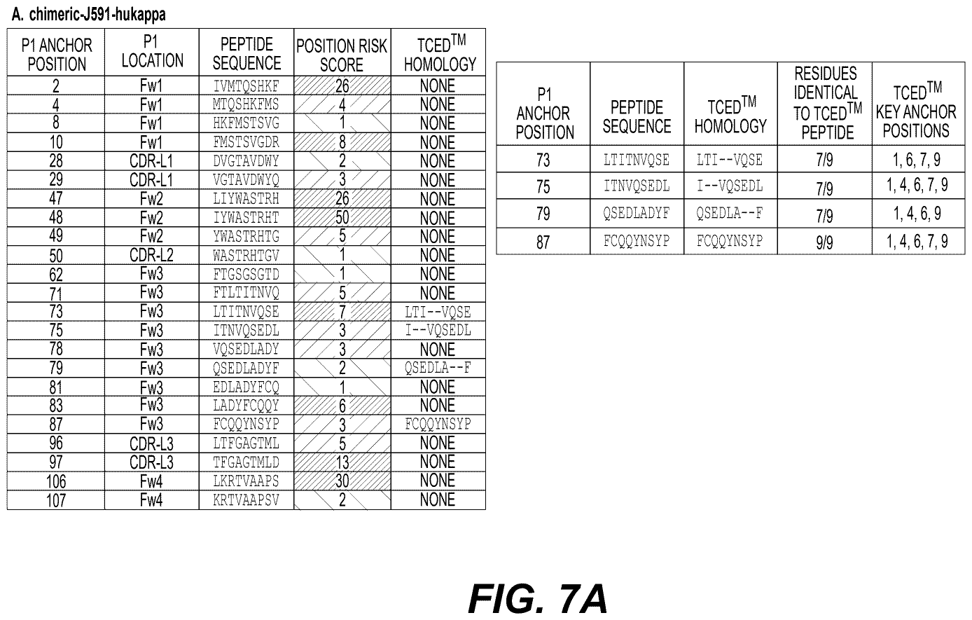

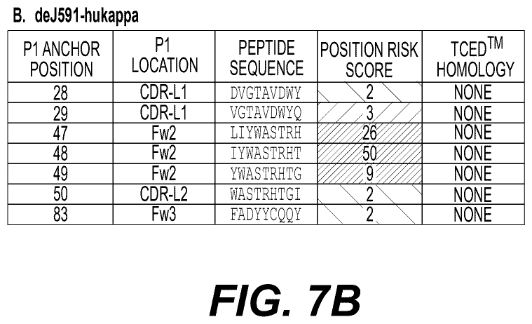

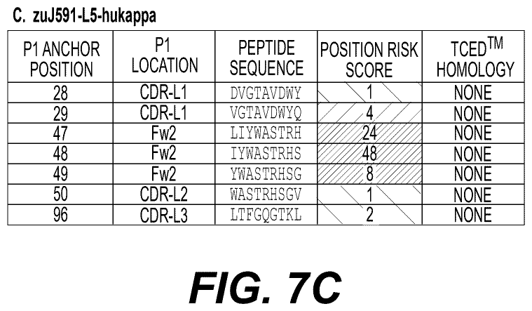

Chimeric mouse J591VK-hukappa contains 23 weak, medium, and strong affinity, non-germline peptides yielding a Total Score of 207 and a Hotspot Max of 50 (FIG. 7). Four peptides were a partial match to previously identified T cell epitopes. Identified peptides were located throughout almost the entire sequence. The “deimmunized” deJ591-hukappa reduced the number of potentially immunogenic peptides to 7 weak, medium, and strong affinity, non-germline peptides yielding a Total Score of 94 and Hotspot Max of 50. No peptides were a partial match to previously identified T cell epitopes. The peptides were located in CDRL1, CDRL2, and FWRL3-CDRL3. Humanization of zuJ591-L5-hukappa showed 7 weak, medium, and strong affinity, non-germline peptides identified, with no partial matches to previously identified T cell epitopes. The peptides in CDRL1 and CDRL2 were the same between deJ591-hukappa and zuJ591-L5-hukappa. However, the increased humanization zuJ591-L4 decreased the Hotspot Score of the peptides in CDRL2, removed the FWR3-CDRL3 peptide in deJ591-hukappa, and generated a new weak binding peptide in CDRL3-FWRL4. The Total Score of zuJ591-hukappa was 88 and the Hotspot Max was 48.

### Example 4. ADC Conjugation and Characterization

### Anti-PSMA ADC Conjugation Methods

anti-PSMA ADCs were prepared as site-specific DAR4 (anti-PSMA-LP1 and anti-PSMA-LP2) conjugating to unpaired cysteine while antibody remains intact, and as random DAR4 (anti-PSMA-LP3) conjugating to cysteine yielded from partial reduction of the antibody. LP4-LP29 evaluated modifications to the linker, and LP30-LP32 evaluated modifications to the payload.

### 1.1 Preparation of Anti-PSMA-LP1 Moiety and Anti-PSMA-LP2 Moiety, Site-Specific Conjugation

A scheme for preparation of anti-PSMA-LP1 moiety conjugation is shown below:

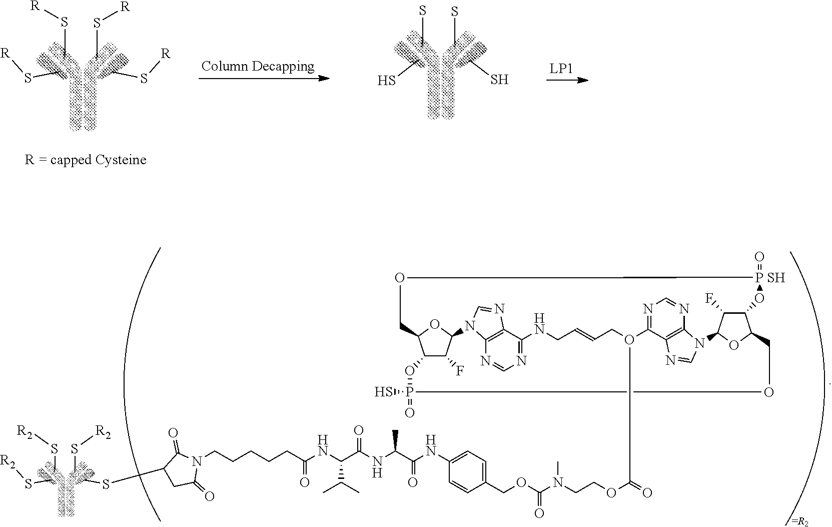

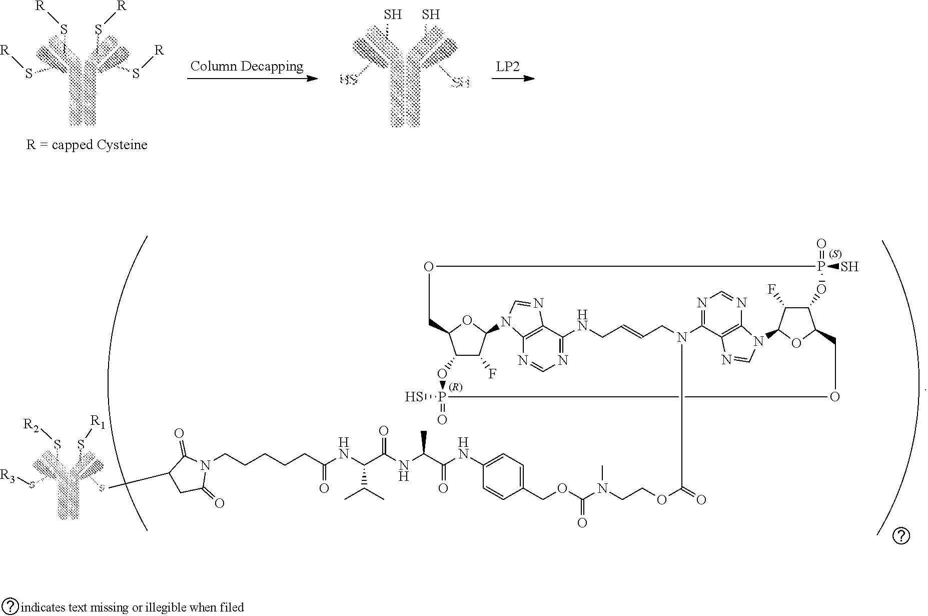

### 1.2 Method

Antibody Decapping: anti-hPSMA-zuJ591-H14L5-hlgG1-A118C-C80 was used for the conjugation. The antibody contains an unpaired cysteine at light chain C80, and heavy chain A118C, both were cysteinylated during antibody production. To enable the cysteine for conjugation, purification was performed using an AKTA Explorer purification platform (GE Healthcare). Appropriate size Mabselect sure column (Cytivia) was equilibrated with 6 column volumes (CV) of 20 mM Sodium Phosphate, 10 mM EDTA, pH 7.2. The conditioned medium containing antibody was filtered through a 0.2 μm membrane and then loaded to the column, followed by washing unbound material with 10 CV of Equilibration buffer. Then either:
A. Column is then washed with 20 mM Sodium Phosphate buffer containing 10 mM EDTA, 10 mM Cysteine, pH 7.2 for 16 hours at a low flow rate. This step was followed by another wash with 20 mM Tris, pH 7.5 for 60 hours at a low flow rate. The sample was eluted using 5 CV of 0.1 M Glycine pH 2.9. Eluted material was loaded on to a 26/10 HiPrep desalting column (GE Healthcare) equilibrated in 1×phosphate-buffered saline (PBS) and eluted in the same buffer. Peak fractions were pooled and filtered. Instead of Desalting, dialysis in 1×PBS may also be used. Antibody concentration was determined using BCA (bicinchoninic acid) assay.
B. The sample was eluted using 5 CV of 0.1 M Glycine pH 2.9. Eluted material was added onto a 26/10 HiPrep desalting column (GE Healthcare) equilibrated in 1× phosphate-buffered saline (PBS) and eluted in the same buffer. Peak fractions were pooled and filtered. Instead of Desalting, dialysis in 1×PBS can also be used. Antibody concentration was determined using BCA assay. To a solution of antibody (10 mg/mL), in 1×DPBS/2 mM EDTA, equal volume of TCEP solution (10 mM in 1×DPBS/2 mM EDTA, pH 7.5) was added. The solution was mixed for 90 minutes before purification by FPLC (fast protein liquid chromatography) using Hitrap desalting into 1×DPBS/2 mM EDTA. Then, dehydroascorbic acid (DHAA) was added to the antibody solution with a final concentration of 1 mM DHAA. The solution was mixed for 1 hour before purification by FPLC using Hitrap desalting into 1×DPBS/2 mM EDTA. Antibody concentration was determined using BCA assay.

Conjugation and purification: To a solution of decapped antibody (5.8 mg/mL, 3.5 mL; or 7.5 mg/mL, 2 mL) in 1×PBS, 2 mM EDTA was added the linker-payload (4.29 molar equivalents from a 10 mM DMSO stock solution of either LP1 or LP2, or salts thereof; or 5 molar equivalents from a 10 mM DMSO stock solution of LP4-LP32, or salts thereof). The conjugation reaction was immediately mixed thoroughly, and conjugation was allowed to proceed at room temperature for a period of 0.5 hour before purification by FPLC using Hitrap desalting (2×5 mL) columns into 25 mM Na Citrate, 100 mM sucrose, pH 6.0. The eluate was pooled, filter sterilized (Whatman Puradisc 13, cat. 6791-1302), and stored at 4° C. The purified ADCs were analyzed for total protein content (bicinchoninic acid assay, Pierce BCA protocol, catalogue #23225). The ADC product was characterized by HPLC-HIC (high-performance liquid chromatography-hydrophobic interaction chromatography), SEC (size exclusion chromatography), and RP-UPLC-MS (reverse phase ultra-performance chromatography-mass spectrometry). The average DAR (drug-antibody ratio) and drug distribution were derived from interpretation of HIC and LC-MS data.

### 1.3 Results

Anti-PSMA-LP2: 2.44 mg/mL, 8 mL. Total 19.52 mg. Percent Yield: 96.2%. Average DAR (HIC-HPLC)=4.12. Percent yield was calculated as [final ADC yield]/[starting mAb]. Starting mAb=5.8 mg/ml×3.5 ml=20.3 mg; final ADC yield=2.44 mg/ml×8 ml=19.52 mg.

Anti-PSMA-LP1: 1.87 mg/mL, 8 mL. Total 14.96 mg. Yield: 73.7%, avg DAR (HIC-HPLC)=4.07. For calculating percent yield: starting mAb=5.8 mg/ml×3.5 ml=20.3 mg, final ADC=1.87 mg/ml×8 ml=14.96 mg.

The conjugation results for anti-PSMA-LP1 and -LP2 are summarized in Table 20, along with the conjugation results for LP4-LP32.

TABLE 20
ADC Conjugation Results

| | Concentration | Volume | Total | % | DAR (HIC- |
|---|---|---|---|---|---|
| Anti-PSMA-LPₓ | (mg/mL) | (mL) | (mg) | Yield | HPLC) |
| LP2 | 2.44 | 8.0 | 19.52 | 96.2 | 4.12 |
| LP1 | 1.87 | 8.0 | 14.96 | 73.7 | 4.07 |
| LP4 | 2.42 | 5.25 | 12.71 | 73.1 | 3.90 |
| LP5 | 2.80 | 5.50 | 15.40 | 88.7 | 3.90 |
| LP6 | 2.79 | 5.50 | 15.35 | 88.3 | 3.92 |
| LP7 | 2.91 | 5.50 | 16.01 | 92.1 | 4.02 |
| LP8 | 1.25 | 5.25 | 6.56 | 37.8 | 4.06 |
| LP9 | 2.93 | 5.50 | 16.12 | 92.8 | 3.96 |
| LP10 | 2.10 | 5.00 | 10.50 | 60.4 | 3.85 |
| LP11 | 2.24 | 4.00 | 8.96 | 51.6 | 2.50 |
| LP12 | 1.66 | 5.50 | 9.13 | 52.6 | 3.96 |
| LP13 | 2.10 | 4.75 | 9.98 | 57.4 | 3.78 |
| LP14 | 1.89 | 4.50 | 8.51 | 49.0 | 3.81 |
| LP15 | 3.09 | 5.50 | 17.00 | 97.8 | 4.04 |
| LP16 | 2.43 | 5.50 | 13.37 | 76.9 | 4.02 |
| LP17 | 1.61 | 5.00 | 8.05 | 53.7 | 3.98 |
| LP18 | 1.79 | 5.00 | 8.95 | 59.7 | 3.92 |
| LP19 | 1.75 | 5.00 | 8.75 | 58.2 | 3.87 |
| LP20 | 2.87 | 5.25 | 15.07 | 86.7 | 3.78 |
| LP21 | 1.68 | 5.00 | 8.40 | 55.8 | 4.17 |
| LP22 | 1.18 | 6.00 | 7.08 | 47.1 | 3.35 |
| LP23 | 1.71 | 5.00 | 8.55 | 56.8 | 3.22 |
| LP24 | 1.14 | 5.00 | 5.70 | 37.9 | 3.82 |
| LP25 | 3.11 | 5.00 | 15.55 | Quantitative | 3.84 |
| LP26 | 2.81 | 5.00 | 14.05 | 93.7 | 3.88 |
| LP27 | 2.04 | 5.00 | 10.20 | 68.0 | 4.02 |
| LP28 | 2.93 | 5.50 | 16.12 | 92.8 | 3.93 |
| LP29 | 1.61 | 5.00 | 8.05 | 53.5 | 3.90 |
| LP30 | 2.37 | 5.50 | 13.04 | 75.0 | 3.96 |
| LP31 | 2.13 | 5.00 | 10.65 | 70.8 | 3.76 |
| LP32 | 1.88 | 5.00 | 9.40 | 62.5 | 3.94 |

### 2.1 Preparation of Anti-PSMA-LP3 Moiety, Random Conjugation

PSMA-STING ADCs were prepared as random DAR4 (anti-PSMA-LP33, LP35-LP36, and LP39-LP53) conjugating to the cysteine yielded from partial reduction of the antibody.

A sample scheme for preparation of PSMA S-attachment (e.g., anti-PSMA-LP3 (LP3) moiety) is shown below:

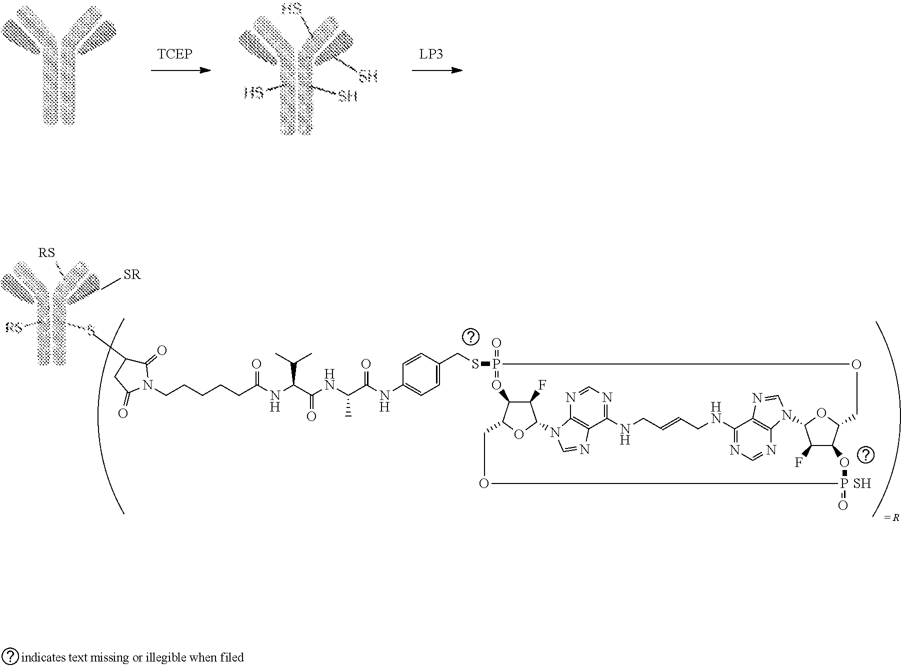

### 2.2 Method

Citrate buffer preparation: To 1 L of DI (deionized) water was added 34.2 g of sucrose and 7.35 g of Na citrate dihydrate. The mixture was stirred and then the pH was adjusted to 6.0 with 1 M HCl.

Conjugation and purification: Anti-hPSMA-zu591-H14L5-IgG1 antibody was used for the conjugation. The antibody purification was performed using an AKTA Explorer purification platform (GE Healthcare). Appropriate size Mabselect sure column (Cytivia) was equilibrated with 6 column volumes (CV) of 20 mM Sodium Phosphate, pH 7.2. The conditioned medium containing antibody was filtered through a 0.2 μm membrane and then loaded to the column, followed by washing unbound material with 10 CV of Equilibration buffer. The sample was eluted using 5 CV of 0.1 M Glycine pH 2.9. Eluted material was loaded onto a 26/10 HiPrep desalting column (GE Healthcare) equilibrated in 1×phosphate-buffered saline (PBS) and eluted in the same buffer. Peak fractions were pooled and filtered. Instead of Desalting, dialysis in 1×PBS may also be used. Antibody concentration was determined using BCA assay as described below.

To a solution of antibody (5 mg/mL, 2.5 mL) in 1×DPBS buffer, 0.5 M EDTA stock solution was added to a final EDTA concentration at 2 mM. TCEP solution from a freshly prepared stock (1 mM) in the same buffer was added at 1:3.336 molar equivalents (mAb: TCEP). The solution was mixed thoroughly and incubated at room temperature (21° C.) for 1 hour. The reduced antibody solution was further diluted with equal volume of 50:50 1×DPBS:propylene glycol, premixed with the LP3 (7.44 molar equivalents from a 10 mM DMSO stock solution), to obtain a solution with a final protein concentration of ~2.315 mg/mL. The conjugation reaction was immediately mixed thoroughly, and conjugation was allowed to proceed at room temperature for a period of approximately 0.5 hour before purification by FPLC using Hitrap desalting (2×5 mL) columns into 25 mM Na Citrate buffer containing 100 mM sucrose, at pH 6.0. The eluate was pooled, filter sterilized (Whatman Puradisc 13, cat. 6791-1302), and stored at 4° C. The purified ADC was analyzed for total protein content (bicinchonic acid assay, Pierce BCA protocol, catalogue #23225). The ADC product was characterized by HPLC-HIC, SEC, and RP-UPLC-MS. The average DAR and drug distribution are derived from interpretation of HIC and LC-MS data.

The negative control ADC anti-SEB-LP3 moiety was prepared based on the protocol above. The anti-SEB monoclonal antibody 79G9 was used for the conjugation. 0.5 M EDTA stock solution was added to a solution of anti-SEB 79G9 antibody (6 mg/mL, 1 mL) in 1×DPBS buffer, to a final EDTA concentration at 2 mM. TCEP solution from a freshly prepared stock (1 mM) in the same buffer was added at 1:3.75 molar equivalents (mAb: TCEP). The solution was mixed thoroughly and incubated at room temperature (21° C.) for 1 hour. The reduced antibody solution was further diluted with equal volume of 50:50 1×DPBS:propylene glycol, premixed with LP3 (7.44 molar equivalents from a 10 mM DMSO stock solution), to obtain a solution with a final protein concentration of ~2.5 mg/mL. The conjugation reaction was immediately mixed thoroughly, and conjugation was allowed to proceed at room temperature for a period of approximately 0.5 hour before purification by FPLC using Hitrap desalting (2×5 mL) columns into 25 mM Na Citrate buffer containing 100 mM sucrose, at pH 6.0. The eluate was pooled, filter sterilized (Whatman Puradisc 13, cat. 6791-1302), and stored at 4° C.

### 2.3 Results

anti-PSMA-LP3: prepared as 1.16 mg/mL, 6 mL, Total 6.96 mg, DAR 3.95, 55.7% yield. For calculating percent yield: starting mAb amount=5 mg/ml×2.5 ml=12.5 mg; ADC yield=1.16 ng/ml×6 ml=6.96 mg.

The conjugation results for anti-PSMA-LP3 are summarized in Table 21, along with the conjugation results for LP33-LP53.

TABLE 21
S-Attachment ADC Conjugation Results

| Anti- | | | | | DAR |
|---|---|---|---|---|---|
| PSMA- | Concentration | Volume | Total | | (HIC- |
| LPₓ | (mg/mL) | (mL) | (mg) | % Yield | HPLC) |
| LP3 | 1.16 | 6.0 | 6.96 | 55.7 | 3.95 |
| LP33 | 1.70 | 9.0 | 15.30 | 97.1 | 4.00 |
| LP35 | 2.04 | 4.2 | 8.57 | Quantitative | 3.75 |
| LP36 | 1.81 | 4.0 | 7.24 | 96.5 | 3.87 |
| LP39 | 2.32 | 7.5 | 17.40 | Quantitative | 3.67 |
| LP40 | 6.17 | 1.5 | 9.26 | Quantitative | 3.69 |
| LP41 | 4.13 | 1.5 | 6.20 | 82.7 | 4.20 |
| LP42 | 1.20 | 3.0 | 3.60 | 72.0 | 4.16 |
| LP43 | 1.98 | 7.5 | 14.85 | Quantitative | 4.31 |
| LP44 | 1.58 | 7.5 | 11.85 | 94.8 | 3.96 |
| LP45 | 1.72 | 8.0 | 13.76 | Quantitative | 4.03 |
| LP46 | 1.48 | 7.5 | 11.10 | 88.8 | 4.40 |
| LP47 | 1.89 | 8.0 | 15.12 | Quantitative | 4.03 |
| LP48 | 1.84 | 8.5 | 15.64 | Quantitative | 4.17 |
| LP49 | 1.30 | 7.5 | 9.75 | 77.9 | 3.83 |
| LP50 | 1.22 | 8.0 | 9.76 | 78.0 | 3.67 |
| LP51 | 1.33 | 8.0 | 10.64 | 85.0 | 3.60 |
| LP52 | 1.15 | 6.0 | 6.90 | 55.2 | 3.88 |
| LP53 | 1.15 | 6.0 | 6.90 | 55.2 | 3.82 |

### 3 Preparation of Anti-PSMA-LP33, Random Conjugation

A scheme for preparation of anti-PSMA-LP33 moiety is shown below:

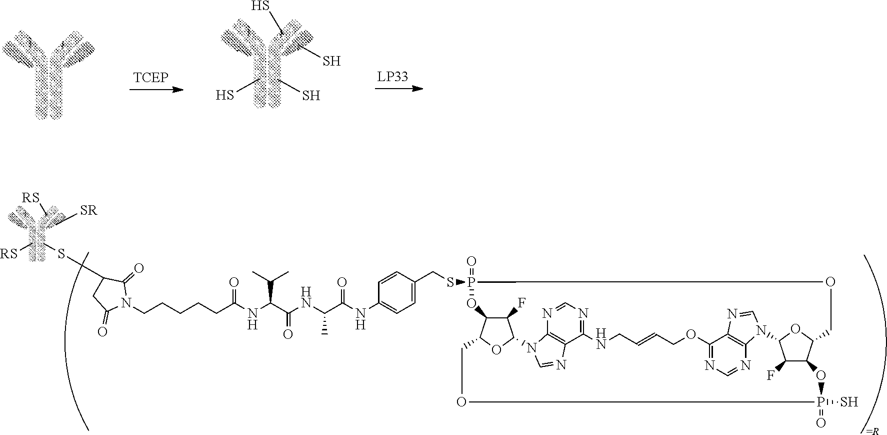

Conjugation and purification: To a solution of antibody (4 mg/mL, 3.5 mL) in 1×DPBS buffer, 0.5 M EDTA stock solution was added to a final EDTA concentration at 2 mM. TCEP solution from a freshly prepared stock (1 mM) in the same buffer was added at 1:4.18 molar equivalents (mAb: TCEP). The solution was mixed thoroughly and incubated at room temperature (21° C.) for 80 minutes. The reduced antibody solution was further diluted with equal volume of 50:50 1× DPBS:propylene glycol, premixed with the LP33 linker-drug conjugate (4.18 molar equivalents from a 10 mM DMSO stock solution), to obtain a solution with a final protein concentration of ~1.79 mg/mL. The conjugation reaction was immediately mixed thoroughly, and conjugation was allowed to proceed at room temperature for a period of approximately 0.5 hour before purification by FPLC using Hitrap desalting (4×5 mL) columns into 1×DPBS. The eluate was pooled, filter sterilized (Whatman Puradisc 13, cat. 6791-1302), and stored at 4° C. The purified ADC was analyzed for total protein content (bicinchoninic acid assay, Pierce BCA protocol, catalogue #23225). The ADC product was characterized by HPLC-HIC, SEC, and RP-UPLC-MS.

Results: anti-PSMA-LP33: 1.70 mg/mL, 8 mL. Total 15.3 mg. Percent Yield: 97.1%, DAR 4.00. For calculating percent yield: starting mAb=4 mg/ml×3.5 ml=14 mg; final ADC=1.7 mg/ml×8 ml=13.6 mg.

### 4 ADC Characterization Methods

### 4.1 Protein Concentration—BCA Assay

Protein concentration was determined using BCA assay (Thermo Scientific, Cat. 23225 or 23227) using a UV/Vis 96-well plate. To 20 μl of serial-diluted ADC sample or bovine gamma globin 2 mg/ml standard, 160 μl of prepared BCA reagent (fresh mixed 200 μL of reagent B and 10 mL of reagent A) was added and samples were mixed thoroughly. Samples were incubated at 37° C. for 30 min. Plates were read at 562 nm on a SpectraMax M5 plate reader (Molecular Devices, S/N MV05371). Data was analyzed using SoftMax Pro software with a 4-parameter fitting model.

### 4.2 HIC-HPLC

Antibody drug conjugates were subjected to hydrophobic interaction chromatography (HIC) on a TSKgel® Butyl-NPR column (Tosoh Bioscience; 4.6 mm×35 mm i.d.; 2.5 μm particle size) connected to an Agilent 1260 series HPLC at room temperature. Samples were injected (10 μL) at or above 1 mg/mL (at 1 mg/mL for all stability samples). A linear gradient elution was employed starting at 100% mobile phase A/0% mobile phase B, transitioning to 0% mobile phase A/100% mobile phase B over a period of 15 min (mobile phase A: 1.5 M ammonium sulfate, 25 mM sodium phosphate at pH 7.0 and mobile phase B: 25% isopropanol, 25 mM sodium phosphate at pH 7.0), at 0.7 mL/min or 0.6 mL/min. Injection of unmodified antibody provided a means of identifying the peak with DAR=0. Antibodies were detected based on absorbance at 280 nm. Peak area was analyzed using Agilent Chemstation software.

DAR (drug-antibody ratio) was calculated as below:

$$Avg DAR = {\sum }_{0}^{8} F ( \% peak area of DAR n * n / 100 )$$

### Example

DAR 0:10%
DAR 2:25%
DAR 4:25%
DAR 6:25%
DAR 8:15%

$$Avg DAR = ( 10 * 0 / 100 ) + ( 25 * 2 / 100 ) + ( 25 * 4 / 100 ) + ( 25 * 6 / 100 ) + ( 15 * 8 / 100 ) = 4.2$$

### 4.3 SEC-HPLC

Antibody drug conjugate aggregation and fragmentation were analyzed using SEC on an Agilent 1200 HPLC system with a DAD (diode array detector). The guard (Agilent AdvanceBio SEC 300 A, 2.7 um, 7.8×50 mm, PL1180-1301) and analytical (Agilent AdvanceBio SEC 300 A, 2.7 um, 7.8×300 mm, PL1180-5301) columns were equilibrated in the mobile phase (0.1 M sodium phosphate, 0.15 M sodium chloride, 5% IPA, pH 7.4) for 3 column volumes. 8 μL of ADC was injected neat and run for 36 minutes at 0.5 mL/min. The ADC was detected at 280 nm with a reference wavelength of 360 nm. Data was analyzed using Agilent Chemstation software (ver. BC.01.07) and reported as % aggregation, % monomer and % fragmentation based on the peak integration results.

### Example 5. In vitro Studies

### 1 PSMA-Specific Binding Analysis

The anti-PSMA antibody, anti-PSMA-LP3 (ADC) and control anti-human IgG1 were conjugated with Alexa Fluor 647 (AF647) succinimidyl ester (Thermo, Cat A20006), at room temperature for 1 hour, at molar ratio of mAb: AF647=1:10, and then purified using an antibody conjugation purification kit (Thermo, Cat A33086), following the manufacturer's instruction.

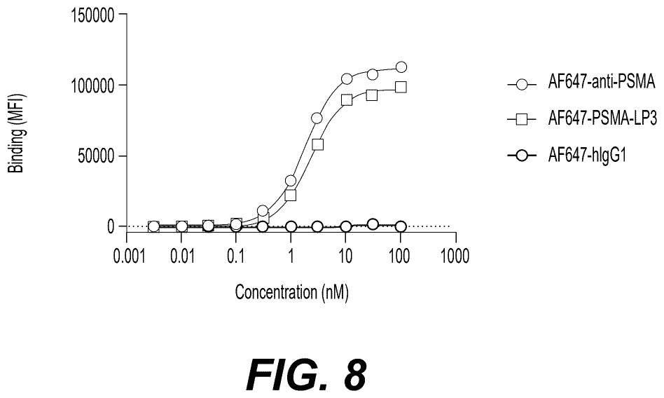

The binding of the ADC to PSMA-expressing cells was measured using BD Fortessa Flow Cytometry. 1×10⁵ LNCaP cells/well were trypsinized, then incubated with antibody, ADC or hlgG1 control that direct labeled with NHS-AF647 on ice for 30 minutes, at the concentration range of 0.003-100 nM, as a semi-log serial dilution. Samples were washed twice using FACS buffer (2% FBS in 1×PBS without calcium and magnesium), then resuspended with 100 μL of FACS buffer containing Fixable Viability dye (1:5000 dilution) and analyzed by flow cytometry. The AF647-anti-PSMA and AF647-PSMA-LP3 showed similar binding affinity to the cells, while AF647-hlgG1 did not bind to the cells (FIG. 8). The results indicate that both the anti-PSMA antibody and the anti-PSMA ADC bind to the PSMA-positive LNCaP cells.

### 2 PSMA-Dependent Antibody-Dependent Cellular Phagocytosis (ADCP) Activity

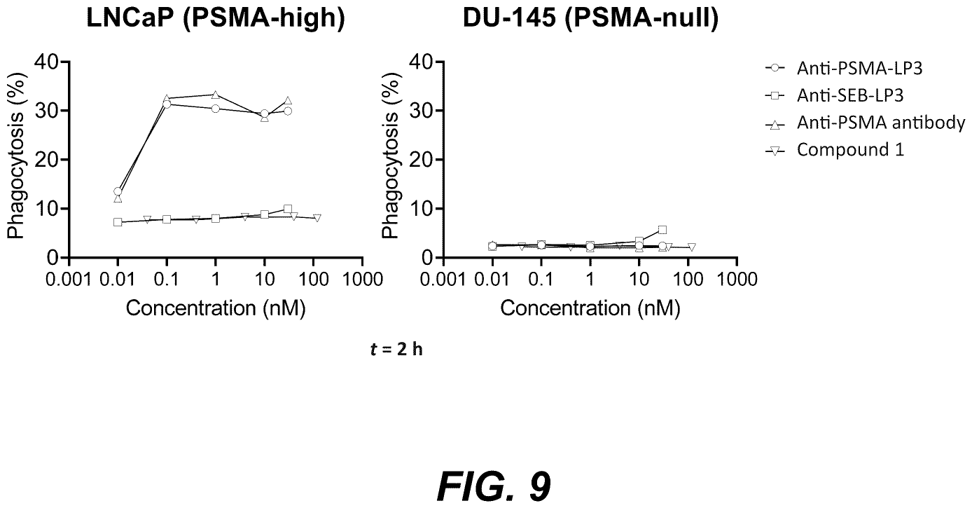

Fresh human monocytes were cultured for 6 days in the presence of M-CSF (macrophage colony-stimulating factor) to differentiate the cells into human macrophages. LNCaP (PSMA-high) and DU-145 (PSMA-null) cells were labeled with carboxyfluorescein diacetate succinimidyl ester (CFSE; Abcam, Cat. ab113853), washed, then seeded in a 96-well U-bottom plate at 1×10⁵ cells per well. Human macrophage cells were added at 1×10⁵ cells per well and mixed well. The test reagents (PSMA-LP3, SEB-LP3 (Staphylococcal enterotoxin B-LP3), PSMA antibody, and Compound 1) were serially diluted and added to the cells at a final concentration ranging from 0.01-100 nM, as indicated (FIG. 9). The plate was incubated at 37° C. for 2 hours. The cells were then analyzed using flow cytometry. Percent phagocytosis was analyzed as CFSE+ (FITC) CD14+ (APC)/[CFSE+/CD14+ and CFSE−/CD14+]×100; CD14 was used as the marker for macrophage cells. Data are reported as the percentage of macrophages that ingested at least one target cell.

As shown in FIG. 9, antigen-dependent phagocytosis was observed in the co-culture of LNCaP and macrophage cells, but not in the co-culture of DU-145 and macrophage cells. Anti-PSMA-LP3 and anti-PSMA antibody promote comparable phagocytosis of tumor cells, and no activity was observed for the negative control ADC (anti-SEB-LP3).

3 Target Cell-Dependent ADC internalization

The anti-PSMA-LP3 ADC was conjugated with pHrodo Red, succinimidyl ester (Thermo, Cat. P36600), at room temperature for 1 hour, at molar ratio of mAb: pHrodo Red=1:10, and then purified using antibody conjugation purification kit (Thermo, Cat A33086), following the manufacturer's instruction.

THP1 cells, C4-2 cells, and mixed C4-2/THP1 cells (C4-2: THP1=1:1) were trypsinized and seeded at 1×10⁵ cells per well in 96-well assay plates, then treated with pHrodo conjugated anti-PSMA-LP3 ADC at a final concentration ranging from 0.001-100 nM ADC, at 37° C., 5% CO₂, for 2 hours. The cells were centrifuged down and washed twice with serum-free medium.

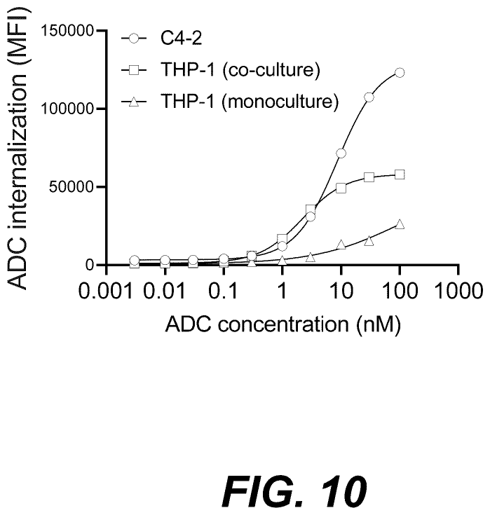

The internalized antibody was measured using NovoCyte Quanteon Flow Cytometer. The ADC internalization was plotted using ADC concentration (X-axis) vs. observed MFI(Y-axis) (FIG. 10). As shown in FIG. 10, internalization was observed in a dose-dependent manner in the C4-2 monoculture and C4-2/THP1 co-culture, with a much lower internalization rate observed for the THP1 monoculture.

### 4 ADCP-Dependent IFN6 Production

C4-2 cells were seeded at 1×10⁵ cells/well (100 μl) in 10% c/RPMI media using 96-well flat-bottom tissue culture plates. Stock solutions of ADCs were serial diluted at 10× of the dilution series, and the 20 μL of ADC solution was added to the wells and incubated at 37° C. for 10 minutes. THP-1 cells were added at 1×10⁵ cells/well (total 80 μL).

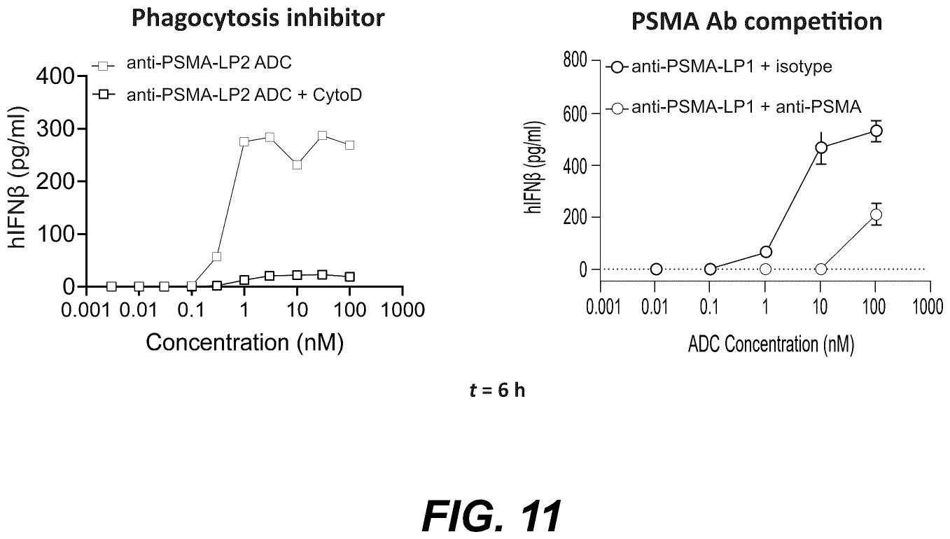

hIFNβ production upon treatment with anti-PSMA ADC was measured in cells treated to block phagocytosis (FIG. 11, left panel). Cytochalasin D (CytoD; Cell Signaling, Cat. 94946S) was dissolved in DMSO to yield a 5 mM stock solution, then added to a semi-log diluted anti-PSMA-LP2 solution, to the final concentration of 10 μM. After 10 minutes incubation at room temperature, THP1 cells were added, and the plate was incubated at 37° C., 5% CO₂ for 6 hours. Then, the cell culture supernatant was harvested for hIFNβ quantitation.

hIFNβ production was also measured in cells treated with anti-PSMA ADC in competition with either anti-PSMA antibody or isotype control (FIG. 11, right panel). The anti-PSMA-LP1 ADC solution was serial diluted in the presence of isotype control (anti-human IgG1) antibody or anti-PSMA antibody (e.g., the same antibody used to generate the anti-PSMA-LP1 ADC), both at 2 μM, then added to the test plate at the final ADC concentration ranging from 0.001-100 nM. The plate was incubated at 37° C., 5% CO₂ for 6 hours. Then the cell culture supernatant was harvested for hIFNβ quantitation.

IFNβ release was measured using Human IFNβ Quantikine ELISA Kit (R&D systems, cat #DIFNB0) following the manufacturer's instruction. At the end of the assay, QuantaBlu Fluorogenic peroxidase substrate (Thermo, Cat #15169) was used to probe the signal. Plates were read on a Molecular Devices M5 plate reader at excitation 320 nM and emission of 405 nM. 5 ADCP-Dependent Myeloid Cell Activation

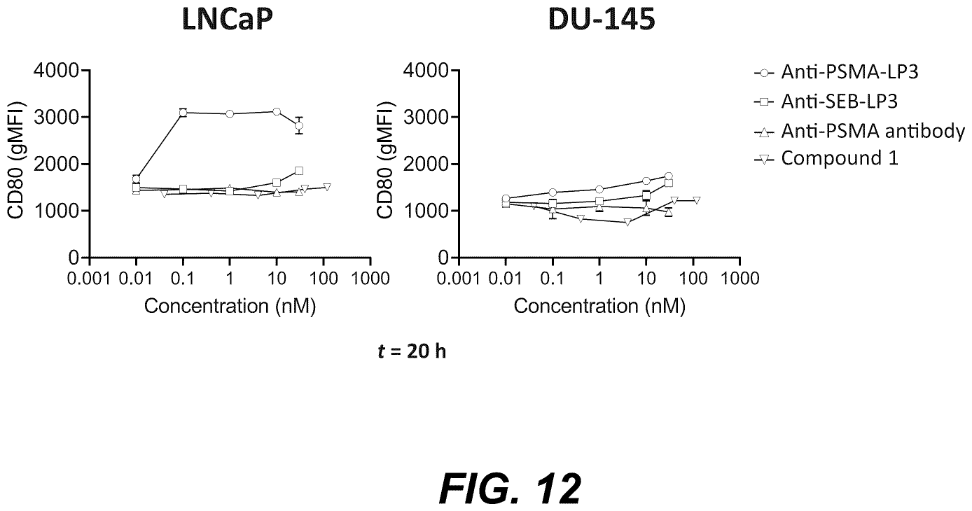

Fresh human monocytes were cultured with M-CSF for 6 days to differentiate into human macrophages, and then harvested for the study. LNCaP or DU-145 cells were trypsinized and seeded at 1×10⁵ cells/well in 96-well plates. Human macrophage cells were added to each well, and then antibody and ADCs were added at the final concentration ranging from 0.01-30 nM for antibodies and ADCs, and 0.04-120 nM for Compound 1 alone. The cell plates were incubated at 37° C., 5% CO₂ for 20 hours, then cells were harvested for flow cytometry analysis. As shown in FIG. 12, CD80 expression in macrophage cells (Y-axis) was plotted vs. ADC concentration (nM).

The anti-PSMA-LP3 ADC activated macrophage cells in the presence of LNCaP cells, as evidenced by the up-regulation of CD80 expression at concentrations as low as 0.1 nM (FIG. 12, left panel). CD80 expression remained unchanged with the treatment of anti-PSMA antibody alone or Compound 1 alone, and only a small effect on CD80 expression was observed at high concentrations of the anti-SEB-LP3 ADC (negative control antibody-Compound 1 conjugate).

The upregulation of CD80 expression was not observed in anti-PSMA-LP3 treated macrophage cells in the presence of DU-145 cells, which do not express PSMA (FIG. 12, right panel).

### Example 6. LNCaP Xenograft Model

### 1 Anti-PSMA-Compound 1 ADC in vivo Anti-Tumor Activity in PSMA+LNCaP Xenograft Model

CB17 SCID mice aged 6-8 weeks were used for this study. LNCaP-FGC (ECACC, Cat 891102117) cells were inoculated subcutaneously at the right flank with 10×10⁶ cells in 0.2 ml of PBS mixed with Matrigel (50:50); the mice were castrated on day 15 after inoculation. The mice were grouped into 10 mice per group, total five groups. Mice were dosed intravenously (IV) with a single dose, and the tumor volume and body weight were monitored for up to 35 days.

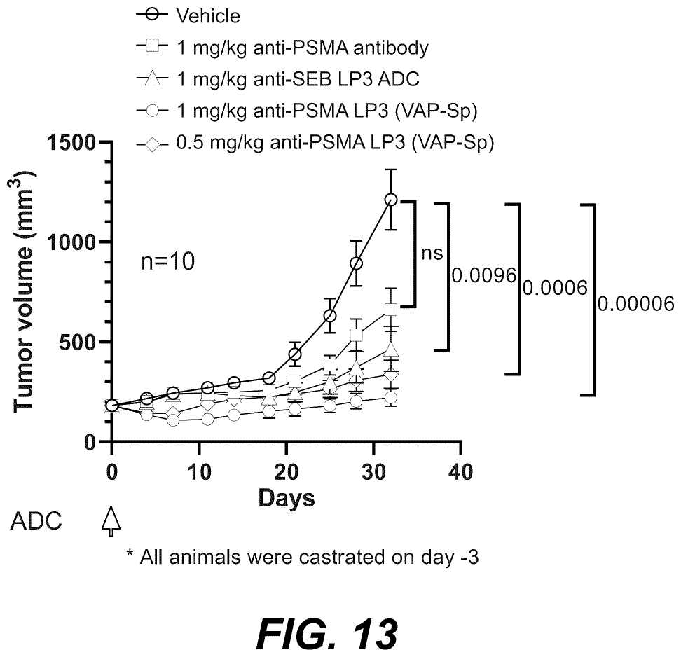

As shown in FIG. 13, the tumor growth was significantly reduced by the anti-PSMA-LP3 ADC (“anti-PSMA-LP3 VAP-Sp”) at 0.5 mg/kg and 1.0 mg/kg. The control anti-SEB-LP3 ADC also showed anti-tumor effect, while no statistical significance was observed between the anti-PSMA antibody alone and vehicle. Two-way ANOVA test was applied for the statistical analysis.

### 2 Modulation of Type I Interferon Genes by Anti-PSMA-LP3 ADC

A pharmacodynamics study was conducted along with the efficacy study described above. An additional 6 mice per group was used for the vehicle, PSMA antibody, anti-PSMA-LP3 ADC, and control SEB-LP3 ADC groups. LNCaP-FGC (ECACC, Cat 891102117) cells were inoculated subcutaneously at the right flank with 10×10⁶ cells in 0.2 ml of PBS mixed with Matrigel (50:50). The mice were castrated on day 15 after inoculation. On day 23, when the average tumor volume reached about 400 mm³, the mice were dosed with a single intravenous dose of antibody or ADC at 1 mg/kg for 6 hours, then tumor and blood were collected from each mouse for RNA-seq analysis.

Tumor samples were preserved in RNAlater for RNA isolation. cDNA library preparation, sequencing, and raw read filtering methods were executed at BGI as described previously (Ren et al., 2012). Reads from the xenograft models were aligned to a combined hg19 and mm10 genome using STAR (see Dobin et al. (2013) Bioinformatics 29 (1): 15-21), and gene isoform counts were quantified by Kallisto (see Bray et. al (2016) Nature Biotechnology 34: 525-527). Gene quantification is given in unit of TPM (transcripts per million).

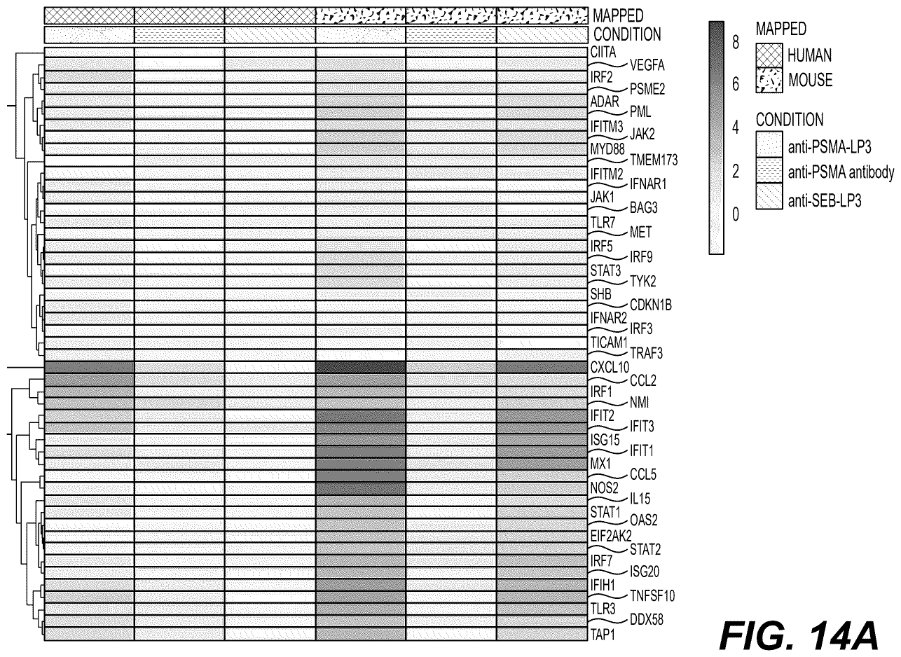

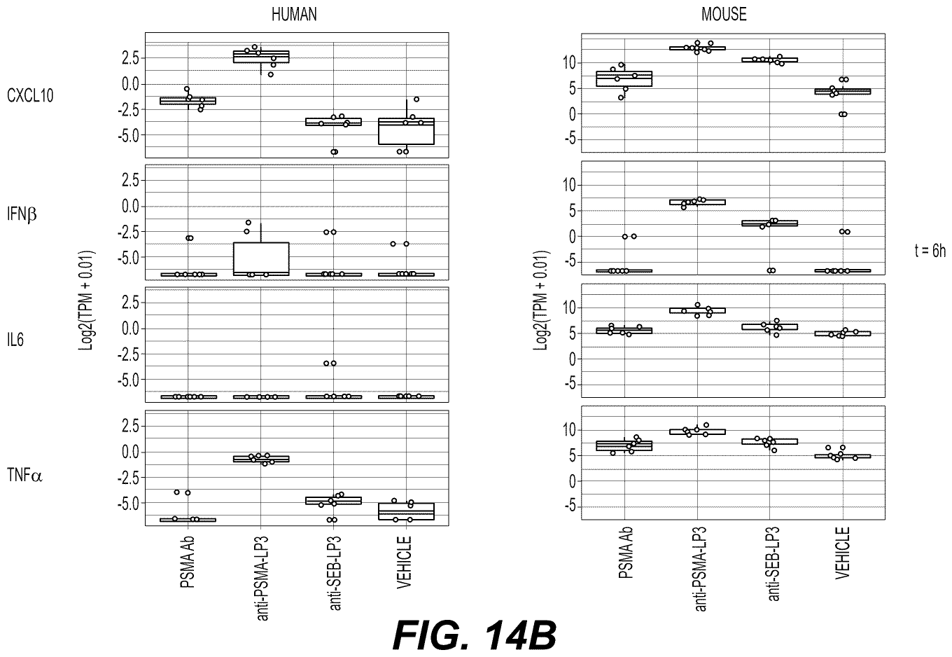

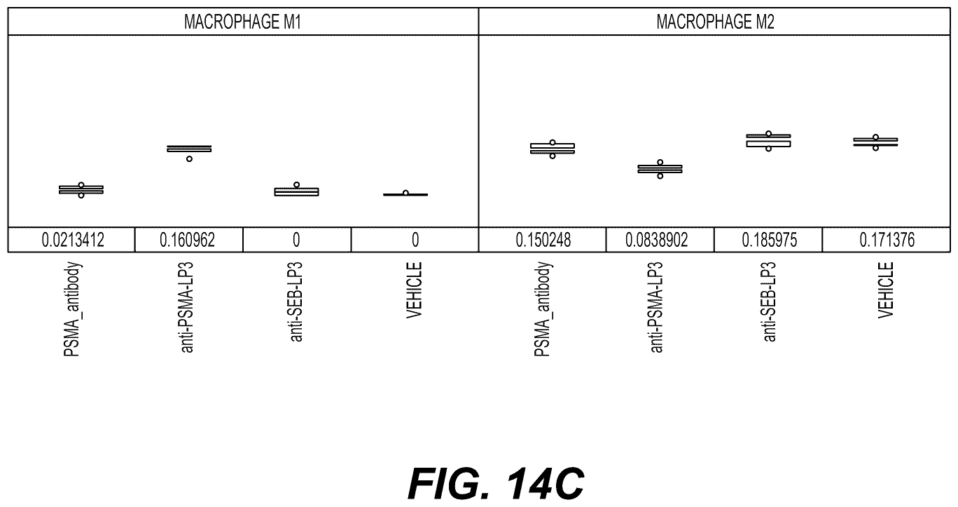

As shown in FIG. 14, four STING-pathway specific cytokines (CXCL10, IFNβ, IL6, TNFα) were modulated by anti-PSMA-LP3 ADC treatment (FIGS. 14A and 14B). Conversion of macrophage polarization from M2 to M1 in the tumor microenvironment was also observed.

### Example 7. 22RV1 Xenograft Model

### 1 22RV1 Xenograft Model: Castrated Condition

Balb/c castrated male nude mice were used for this study. 2.5×10⁶ of 22RV1 cells suspended in 100 μL of 1×PBS containing 50% Matrigel were implanted subcutaneously under the right arm of each mouse, and tumor growth was monitored. The mice were randomized into six groups (8 mice per group) and received treatment as indicated below when tumor size reached about 210 mm³ (212.52 to 216.42 mm³). Mice were treated intravenously for each treatment, either once a week for five weeks (Q7D×5), or with a single dose. Tumor size and body weight were monitored twice a week.
Group 1, Vehicle (IV) Q7D×5
Group 2, 1 mg/kg anti-PSMA antibody (IV) Q7D×5
Group 3, 1 mg/kg anti-SEB-LP3 ADC (IV) Q7D×5
Group 4, 1 mg/kg anti-PSMA-LP3 ADC (IV) Q7D×5
Group 5, 1 mg/kg anti-PSMA-LP3 ADC (IV) Single dose
Group 6, 2 mg/kg anti-PSMA-LP3 ADC (IV) Single dose

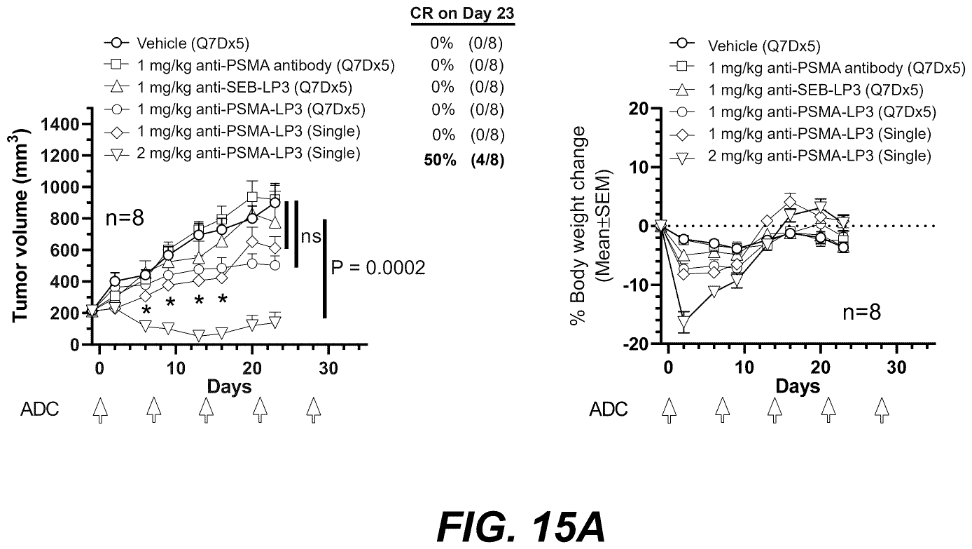

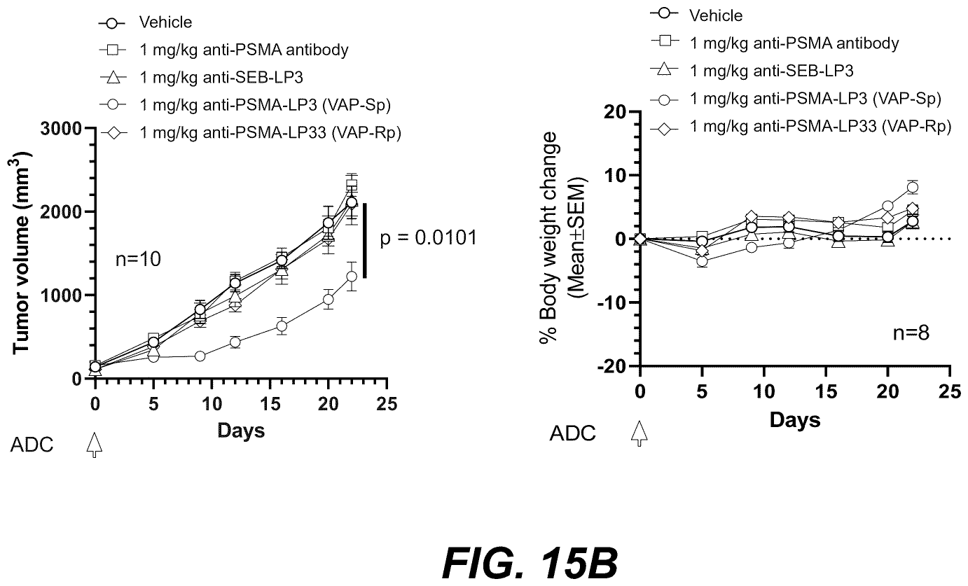

At day 23, significant anti-tumor effect was observed in mice that received the single-dose 2 mg/kg PSMA-LP3 ADC treatment (FIG. 15A). A transient body weight reduction was observed in the PSMA-LP3 ADC treatment groups that recovered over time.

### 2 22RV1 Xenograft Model: Non-Castrated Condition

Balb/c nu/nu male mice were used for this study. 5×10⁶ of 22RV1 cells suspended in 100 μL of 1×PBS containing 50% Matrigel were implanted subcutaneously in the right flank of each mouse and tumor growth was monitored. The mice were randomized into five groups (8 mice per group) and received treatment as indicated below when tumor size reached about 100 mm³ (110.4 to 111.4 mm³). The mice were treated with a single intravenous dose at 1 mg/kg dose, and the tumor size and body weight were monitored for 23 days.
Group 1, Vehicle (IV) Single dose
Group 2, 1 mg/kg anti-PSMA antibody (IV) Single dose
Group 3, 1 mg/kg anti-SEB-LP3 ADC (IV) Single dose
Group 4, 1 mg/kg anti-PSMA-LP3 ADC (IV) Single dose
Group 5, 1 mg/kg anti-PSMA-LP33 ADC (IV) Single dose

Anti-PSMA-LP3 ADC treatment showed statistical tumor growth inhibition at the single intravenous dose of 1 mg/kg (FIG. 15B). The negative control anti-SEB-LP3 ADC and anti-PSMA antibody showed no effect on tumor growth. Minimal body weight change was observed at 1 mg/kg in all conditions.

### Example 8. ADC stability: pAB analogs

### 1 In Vivo Efficacy of S-Linker Payload in 22RV1 Model

Anti-tumor activity of PSMA-LP3 ADC conjugated to various linker-altered pAB analogs was evaluated at 2 dose levels per ADC in Balb/c nude male mice bearing subcutaneous 22RV1 human prostate carcinoma tumors.

22RV1 cells at 2.5×10⁶ cells in 1×PBS containing 50% Matrigel (100 μL) was subcutaneously injected in the right flank of each mouse. Tumor growth was monitored to reach a mean of 100-150 mm³ for the treatment. Mice were randomized into eleven groups (8 mice per group) for the single intravenous dose as shown below. Tumor size and body weight were measured twice per week until day 21.
Group 1, Vehicle (PBS) (IV), single dose
Group 2, 1 mg/kg Anti-PSMA-LP3 (IV), single dose
Group 3, 2 mg/kg Anti-PSMA-LP3 (IV), single dose
Group 4, 1 mg/kg Anti-PSMA-LP49 (IV), single dose
Group 5, 2 mg/kg Anti-PSMA-LP49 (IV), single dose
Group 6, 1 mg/kg Anti-PSMA-LP53 (IV), single dose
Group 7, 2 mg/kg Anti-PSMA-LP53 (IV), single dose
Group 8, 1 mg/kg Anti-PSMA LP44 (IV), single dose
Group 9, 2 mg/kg Anti-PSMA LP44 (IV), single dose
Group 10, 1 mg/kg Anti-PSMA-LP40 (IV), single dose
Group 11, 2 mg/kg Anti-PSMA-LP40 (IV), single dose

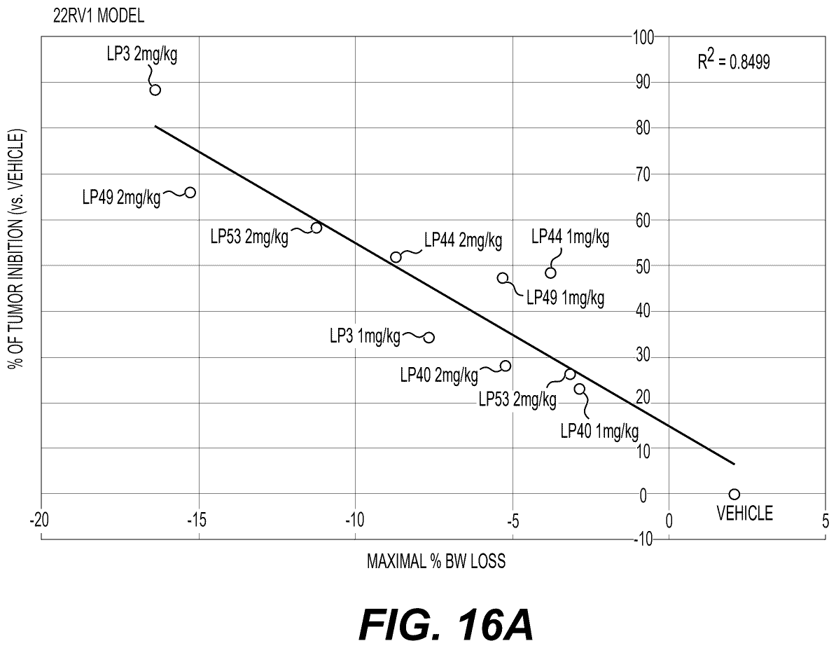

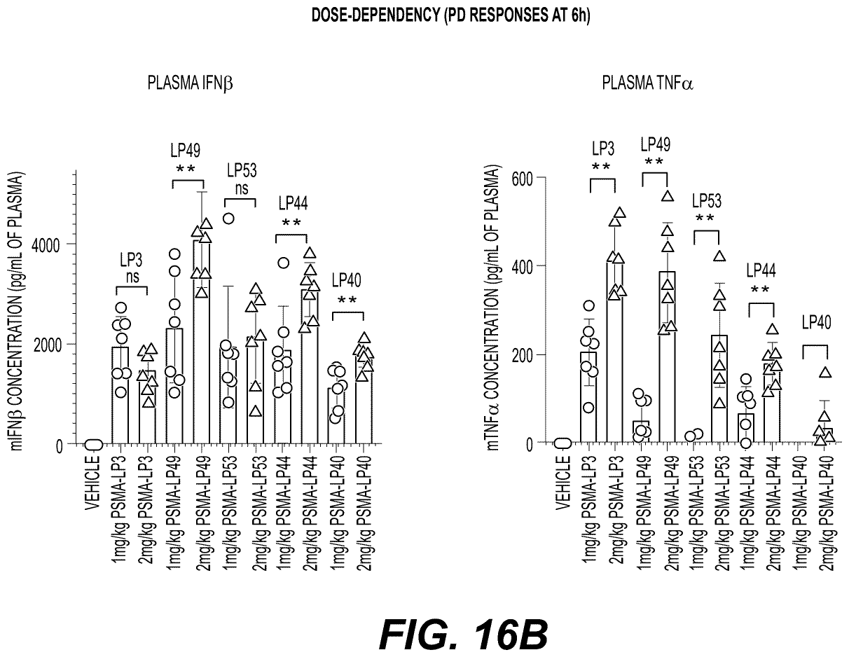

The average tumor size for each group at the end of the study was calculated, and the percent tumor inhibition was calculated as [tumor size (vehicle)−tumor size (treatment)]/tumor size (vehicle)×100. The maximal percent body weight loss (% BW loss, X-axis) vs. percent of tumor inhibition (vs. vehicle) was plotted (FIG. 16A). The linker-payloads of LP3, LP49 and LP53 were identified as the preferred linker-payload options from among the ten tested linker-payloads, as they showed high tumor inhibition activity with low body weight loss in vivo.

### 2 Dose Dependency with S-linker payload

Dose response activity was also evaluated in the study described above. Six hours post injection, blood was collected from each mouse tail, transferred into a Lithium Heparin tube, and centrifuged at 2000 g for 20 min at 4° C. After centrifugation, the plasma was collected at −20° C. for testing.

Mouse TNFα and IFNβ were measured. Plasma IFNβ was measured using Quantikine mouse IFNβ ELISA kit (R&D systems, Cat No. MIFNB0) with the assay range at 15.6-1000 μg/ml. Plasma TNFα was measured using Quantikine mouse TNFα ELISA kit (R&D systems, Cat No. MTA00B) with the assay range at 10.9-700 μg/ml, following the manufacturer's instruction.

All the anti-PSMA ADC treatment groups showed increased cytokine release of TNFα and IFNβ compared with the vehicle groups (FIG. 161). Dose-dependent TNFα release was also observed.

### 3 Further Characterization of pAB analogs in ACs

### 3.1 Buffer Stability Study of Linker-Payloads

A solution of each linker-payload as shown in Table 22 in DMSO (10 mM, 4 μL) was treated with a freshly prepared solution of N-acetyl cysteine in DMSO (3.3 mM, 36 μL). The resulting mixture was incubated at 37 BC for 5 minutes to complete capping of the maleimide. Then, 20 μl of the resulting linker-payload solution was added into 980 μL of pre-warmed D-PBS (pH 7.4) at 37° C. While the resulting solution was incubated at 37° C. at 350 rpm, 125 μL aliquot was taken at each time point (t=0, 6, 24, 48, 120, and 168 hours) and mixed with 25 μL of internal standard solution. Resulting analytical samples were stored at −20° C., and later warmed to 4° C. and analyzed by UPLC.

| System: | Waters H class Acquity UPLC | |
|---|---|---|
| Column: | Waters Acquity UPLC HSS T3, 2.1 × 50 mm packed with 1.8 μm particles. | |
| Temperature: | 50° C. | |
| Flow rate: | 0.9 mL/min | |
| Mobile phase A | Mobile phase A: 0.1% formic acid (98% pure MS spec grade) in water | |
| Mobile phase B | Mobile Phase B: 0.1% formic acid (98% pure MS spec grade) in Acetonitrile | |
| | Time (min) | %-Solvent A |
| Gradient: | Initial | 95 |
| | 0.75 | 95 |
| | 1.00 | 10 |
| | 1.01 | 10 |
| | 1.5 | 0 |

| Injection volume: | 4 μL injection. |
|---|---|
| Detection: | UV max absorbance, ELSD and MS total ion count and extracted mass |
| MS scan: | Positive and negative ion 100 to 1800 amu |
| Data acquisition | 1.5 min |
| time: | |
| Run time: | 1.5 min |

Half-life was then calculated as follows. Time course of the UV area % (260 nm) of the parental linker-payload peak compared with the internal standard peak was used for the half-life calculation. Sampling time points and the remaining parental peaks at each time were plotted and the curve was fit to the equation N(t)/N₀=e^{-lt}. The half-life was calculated with decay constant I with the following equation:

$${T}_{1 / 2} = ln ( 2 ) / λ$$

Internal standard sample: 10 μL of the 10 mM DMSO solution of 4-[({[(2S,5S,6S)-6-[(2E,4E,6S)-7-[(2S,3S)-3-[(2R,3R,4R)-4-hydroxy-3-methoxypentan-2-yl]-2-methyloxiran-2-yl]-6-methylhepta-2,4-dien-2-yl]-5-methyloxan-2-yl]methyl}carbamoyl)amino]butyl 4-acetylpiperazine-1-carboxylate was diluted with 990 μL of 1:1 acetonitrile: D-PBS (pH 7.4).

### 3.2 Calculation of Percent Free Payload

All ADCs were evaluated in the thermal stability at 37° C. Samples were prepared by diluting ADCs to 1 mg/mL in 25 mM Na Citrate buffer containing 100 mM sucrose, at pH 6.0. The samples were then stored at 37° C. for up to either 336 hours or 504 hours. At each time point (T=0, 24, 48, 72, 168, 336, 504 hours), an aliquot was removed and stored at −80° C. until analysis. Samples were analyzed by HIC-HPLC and SEC-HPLC. DAR is reported from the HIC-HPLC analysis.

The released free payload was analyzed from the day 14 (336 hours) sample using the LC-MS method below. % free payload=100× quantified free payload concentration (T14d, 37° C.) /total payload concentration, where total payload concentration is calculated based on the ADC concentration and DAR, at T=0.

Quantification of Compound 1 was performed by LC-MS. Compound 1 reference standard (powdered reagents) were dissolved in LC-MS grade water. Compound 1 standard curve was also prepared in LC-MS grade water. ADC samples were diluted 10× in LC-MS grade water. 10 μL of each sample was injected for LC-MS analysis. Quantitation was done using TargetLynx (Waters)

LC-MS method parameters are shown below:

### Instrumentation:

| | |
|---|---|
| UPLC | Waters Acquity I class plus UPLC |
| MS | Waters Xevo TQ-XS Triple Quadrupole MS |
| Column | Waters Acquity UPLC BEH C18 |
| | 2.1 × 50 mm, 130 Å, 1.7 μm, Part No. 186002350 |
| Mobile phase A | 5 mM ammonium bicarbonate in H₂O |
| Mobile phase B | 5 mM ammonium bicarbonate in MeOH |
| Injection solvent | H₂O |

### LC-MS Method:

Sample
preparation

| Standards in | Serial dilution with injection solvent | | | | |
|---|---|---|---|---|---|
| solution | | | | | |
| Samples in | Solid phase extraction (SPE) on Waters Oasis HLB 96-well μElution Plates | | | | |
| matrix | (186001828BA) | | | | |
| | Wash: 5% MeOH, Elute: ACN/MeOH = 60/40 | | | | |
| Inlet method | Compound 1-NH4 20210927_20% B | | | | |
| Column | 40° C. | | | | |
| temperature | | | | | |
| Sample | 15° C. | | | | |
| temperature | | | | | |
| Injection volume | 5 μL | | | | |
| | Time (min) | Flow (mL/m) | % A | % B | Curve |
| Gradient | 0.0 | 0.5 | 95.0 | 5.0 | 6 |
| | 2.0 | 0.5 | 80.0 | 20.0 | 6 |
| | 2.1 | 0.5 | 10.0 | 90.0 | 6 |
| | 4.0 | 0.5 | 10.0 | 90.0 | 6 |
| | 4.1 | 0.5 | 95.0 | 5.0 | 6 |
| | 6.0 | 0.5 | 95.0 | 5.0 | 6 |

| | MRM Compound 1 and mP-Compound 1 with IS 20210927 | | | | | |
|---|---|---|---|---|---|---|
| MRM method | Mass | Parents | Daughters | Cone Volt. | Colli. Eng. | Dwell |
| Compound 1- | | 747.0700 | 171.0000 | 90 | 72 | 0.025 |
| NH4 | | | | | | |
| | | 747.0700 | 187.9700 | 90 | 64 | 0.025 |
| D6 Compound 1 IS | | 753.2700 | 194.1100 | 100 | 56 | 0.025 |

| Event | Divert to the waste at 2.2 min | |
|---|---|---|
| MS tune file (ES+) | Tune Compound 1-NH4 20210901 | |
| | Capillary (kV) | 2.1 |
| | Sample Cone (V) | 93 |
| | Desolvation Temp (° C.) | 600 |
| | Desolvation Gas (L/h) | 1000 |
| | Cone Gas (L/h) | 150 |
| | Nebuliser (Bar) | 7.0 |
| | LM Resolution 1 | 2.79 |
| | HM Resolution 1 | 14.29 |
| | Ion Energy 1 | −0.8 |
| | LM Resolution 2 | 2.46 |
| | HM Resolution 2 | 14.45 |
| | Ion Energy 2 | 0.2 |
| | Collision gas Flow (mL/min) | 0.16 |
| | Collision | 4 |
| TargetLynx method | TargetLynx Compound 1 and mP-Compound 1 with IS 20220105 | |
| | Retention Time (min) | |

| | |
|---|---|
| Compound 1 | 1.70 |
| D6 Compound 1 | 1.70 |

### 3.3 In Vitro Potency Assay

The method for in-vitro IFNβ production (reported as AUC) was as follows. LNCap cells were seeded in 96 well plates at 60,000 cells/well in 100 μL of cell culture medium (complete RPMI-10% FBS), then 20 μL of ADC was added at the semi-log serial dilution from 0.01-100 nM, mixed well, and incubated at 37° C., 5% CO₂ for 10 minutes, 60,000 cells/well of THP1 cells in 80 μL medium were added to the well, and the cell plate was incubated at 37° C., 5% CO₂ for 20 hours. The plate was centrifuged at 1500 rpm for 5 minutes, supernatant was collected, then stored at −20° C. for the hIFNβ measurement.

The level of hIFNβ was tested using human IFNβ Quantikine kit (R&D systems, Cat #DIFNB0) following manufacturer's instruction. The supernatant was diluted 1:5 using 1×PBS for the assay. The dynamic range of hIFNβ is 7.8-500 μg/ml. The IFNβ production curve was plotted as concentration (X-axis) vs. IFNβ release (Y-axis), curve was fitted as nonlinear regression fit, and AUC (area under curve) was calculated using GraphPad Prism software.

The Ratio of IFNβ production (RIP) was calculated as AUC (area under curve) of IFNβ production for each ADC divided by the AUC of IFNβ production from anti-PSMA-LP2.

The in vitro potency was reported as RIP/DAR.

The results for the further characterization of pAB analogs in ADCs are shown in Table 22. The structure of pAB analogs within a linker-LP3 conjugate is shown below:

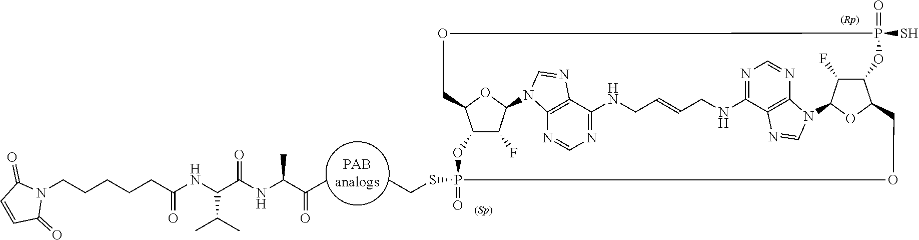

TABLE 22

| | | | | | % free | |
|---|---|---|---|---|---|---|
| | | | | | payload | |
| | | | | | (T_{14d} 37° C. | in vitro |
| | | | Linker | t_{1/2} hr | citrate | potency |
| Linker- | | Linker | Attachment | (37° C. | buffer, pH | assay |
| payload | PAB analogs | property | site | PBS) | 6.0) | RIP^{&}/(DAR) |
| LP42 | 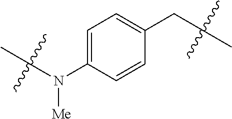 [SMILES: *Cc1ccc(N(*)C)cc1] | Non- cleavable | Sp- thiophosphate (S14) | 111 | 12% | 0.01 (4.31) |
| LP39 | 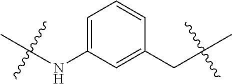 [SMILES: *Cc1cccc(N*)c1] | | | 161 | 10% | 0.01 (3.67) |
| LP3 | 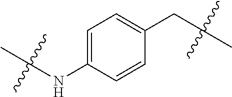 [SMILES: *Cc1ccc(N*)cc1] | Cleavable | | 46 | 56% | 1.00 (3.75)* |
| LP53 | 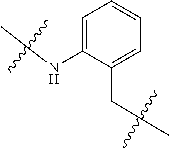 [SMILES: *Cc1ccccc1N*] | | | >168 | 17% | 0.45 (3.82) |
| LP40 | 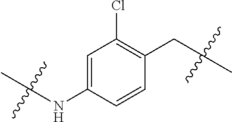 [SMILES: *Cc1ccc(N*)cc1Cl] | | | 101 | Not tested | 0.31 (3.69) |
| LP49 | 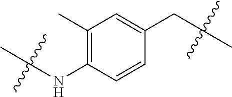 [SMILES: *Cc1ccc(N*)c(C)c1] | | | 98 | 31% | 2.48 (3.83) |
| LP43 | 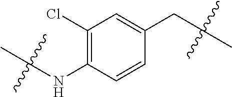 [SMILES: *Cc1ccc(N*)c(Cl)c1] | | | 91 | 19% | 1.15 (4.31) |
| LP45 | 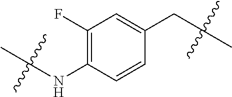 [SMILES: *Cc1ccc(N*)c(F)c1] | | | 85 | 25% | 1.42 (4.03) |
| LP51 | 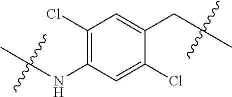 [SMILES: *Cc1cc(Cl)c(N*)cc1Cl] | | | 64 | 31% | 1.36 (3.6) |
| LP44 | 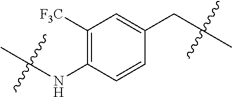 [SMILES: *Cc1ccc(N*)c(C(F)(F)F)c1] | | | 57 | 26% | 0.75 (3.96) |
| LP46 | 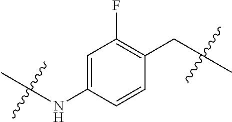 [SMILES: *Cc1ccc(N*)cc1F] | | | 45 | 42% | 1.50 (4.4) |
| LP48 | 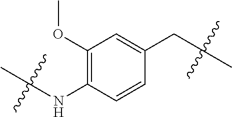 [SMILES: *Cc1ccc(N*)c(OC)c1] | | | 42 | 49% | 1.26 (4.17) |
| LP52 | 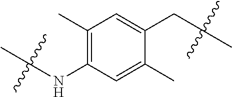 [SMILES: *Cc1cc(C)c(N*)cc1C] | | | 29 | 75% | 1.35 (3.88) |
| LP47 | 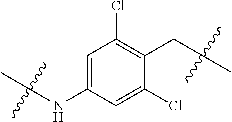 [SMILES: *Cc1c(Cl)cc(N*)cc1Cl] | | | 23 | 33% | 1.08 (4.03) |
| LP50 | 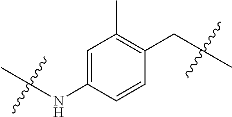 [SMILES: *Cc1ccc(N*)cc1C] | | | 22 | 81% | 1.81 (3.67) |
| LP41 | 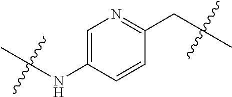 [SMILES: *Cc1ccc(N*)cn1] | | | 6 | Not tested | 0.74 (HER2) |
| LP33 | 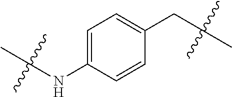 [SMILES: *Cc1ccc(N*)cc1] | | Rp- thiophosphate (52) | Decomp | Not tested | 0.18 (4.2) |

^{&}RIP = Ratio of IFNβ Production AUC ratio to the reference (ADC (LP3).
DBDE = delta bond dissociation energy

### Example 9. ADC Stability: Linker-Drug Attachment

### 1 ADC Buffer Stability Method and Results

### 1.1 N-Attached Linkers

Each ADC was diluted to 1 mg/mL in 25 mM Na Citrate buffer containing 100 mM sucrose, at pH 6.0. The samples were then stored at 37° C. for up to 336 hours (for S-attached linkers) or 504 hours (for N-attached linkers). At each time point (T=0, 24, 48, 72, 168, 336, and 504 hours), an aliquot was removed and stored at −80° C. until analysis. Samples were analyzed by HIC-HPLC and SEC-HPLC. HIC-HPLC and SEC-HPLC methods are described above. DAR was calculated by HIC-HPLC. % DAR change was reported as:

% DAR change=100*DAR at Tₙ/DAR at T₀

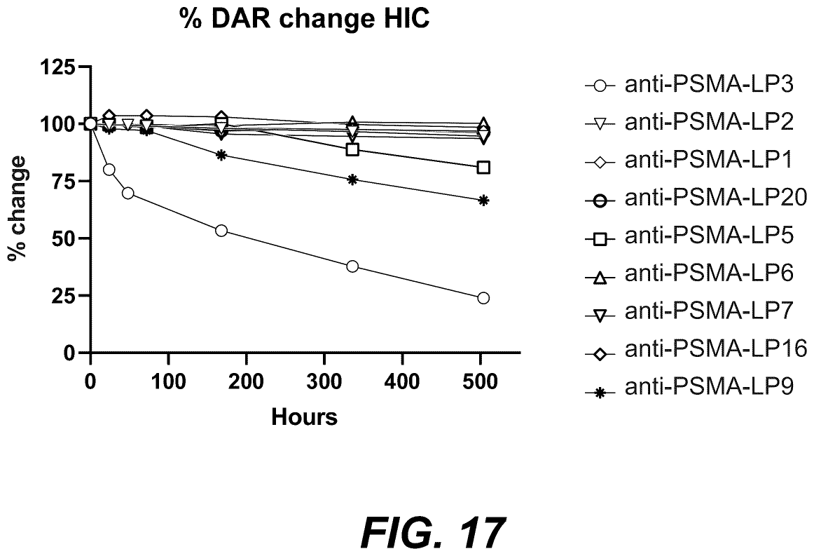

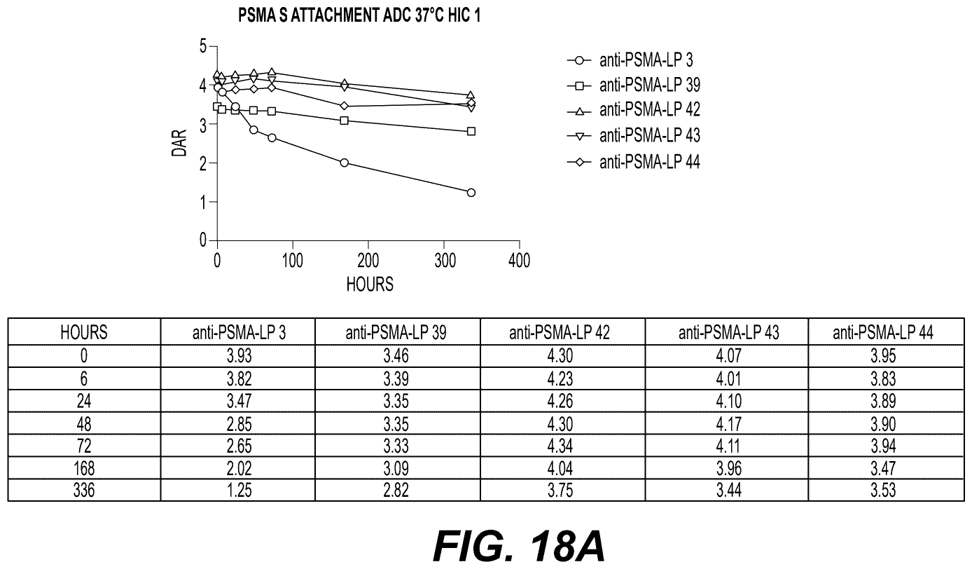

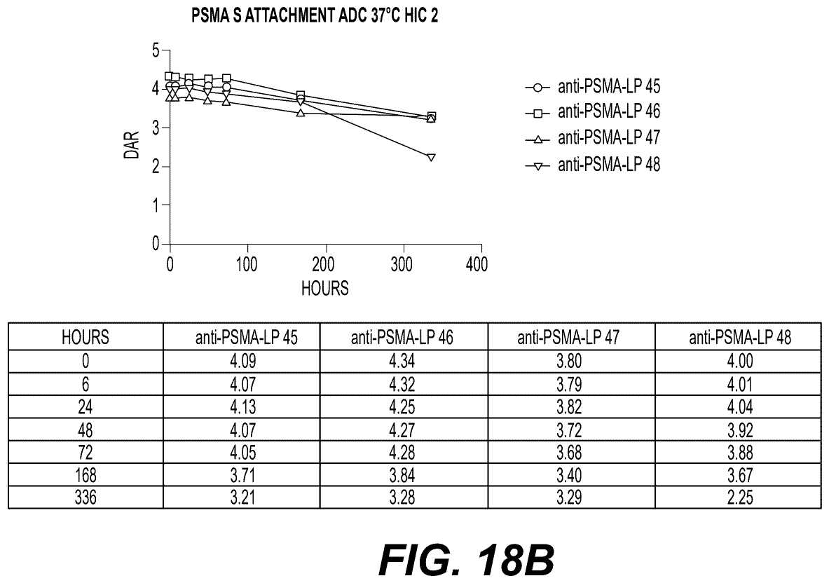

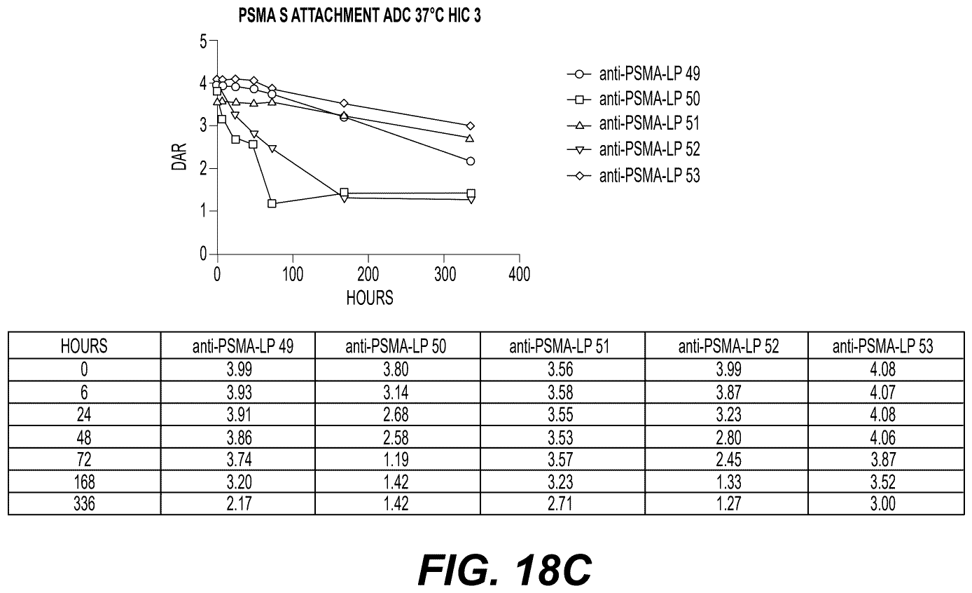

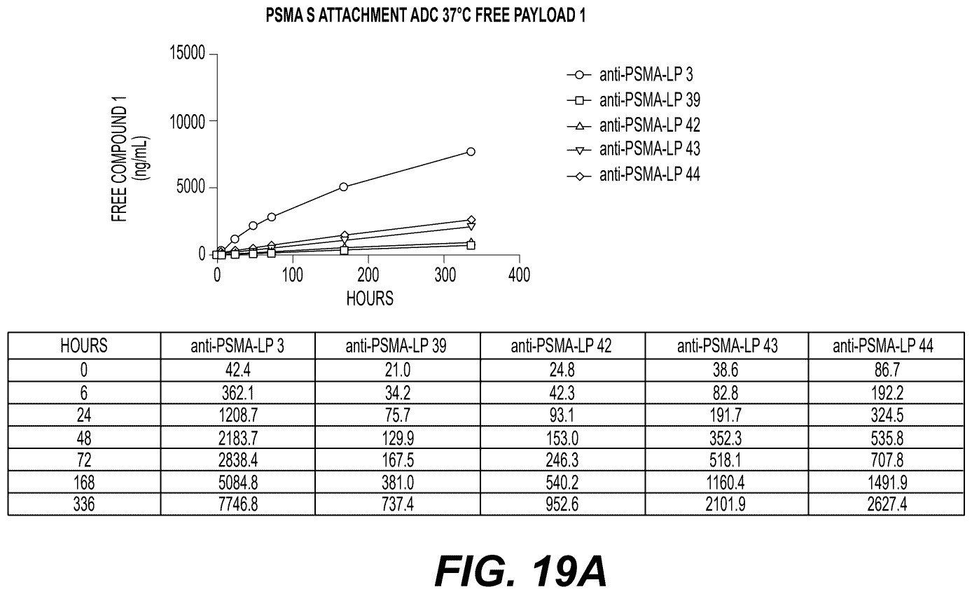

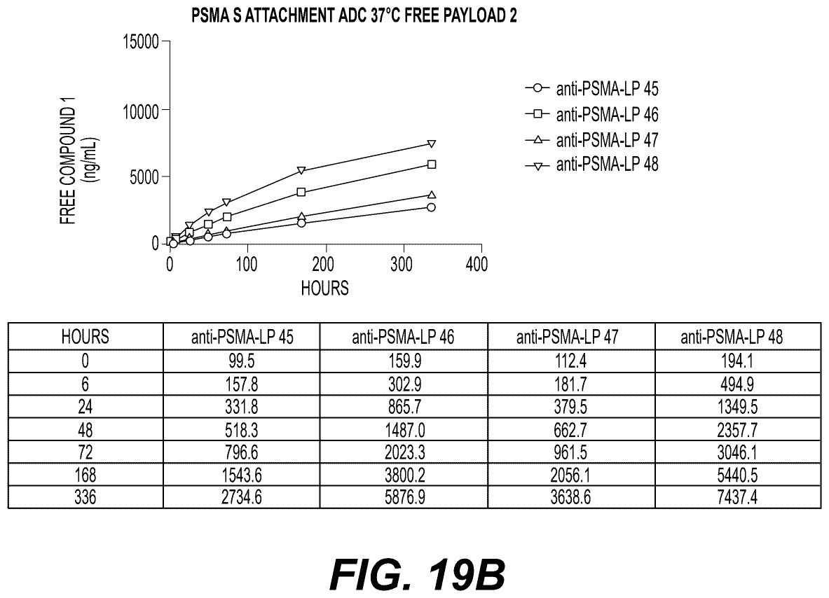

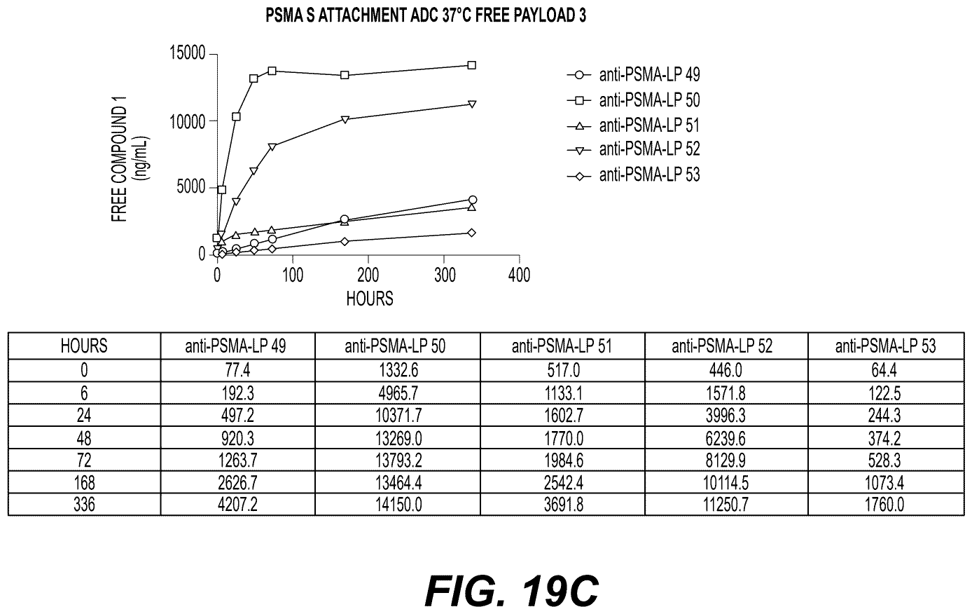

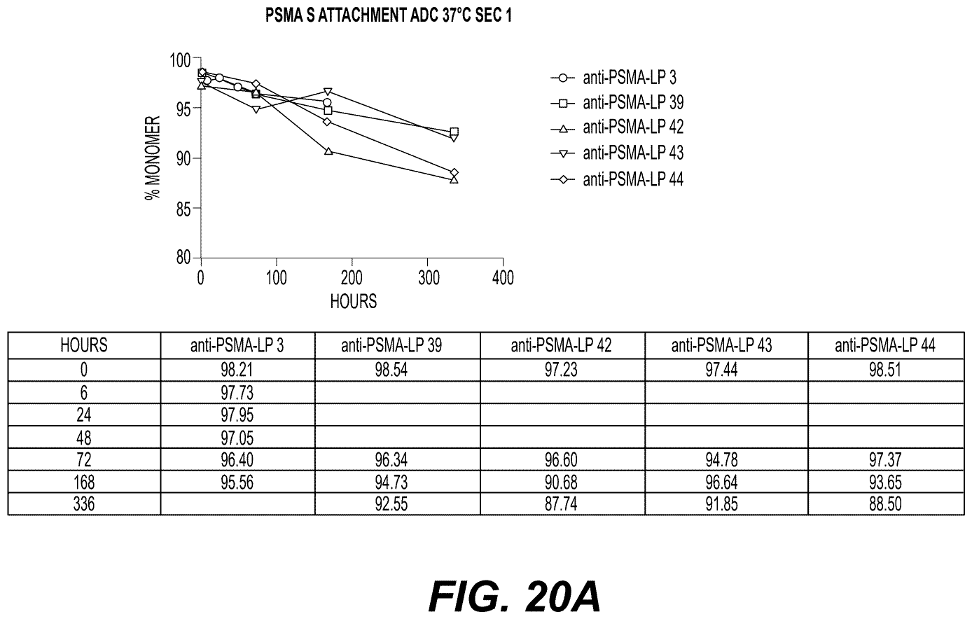

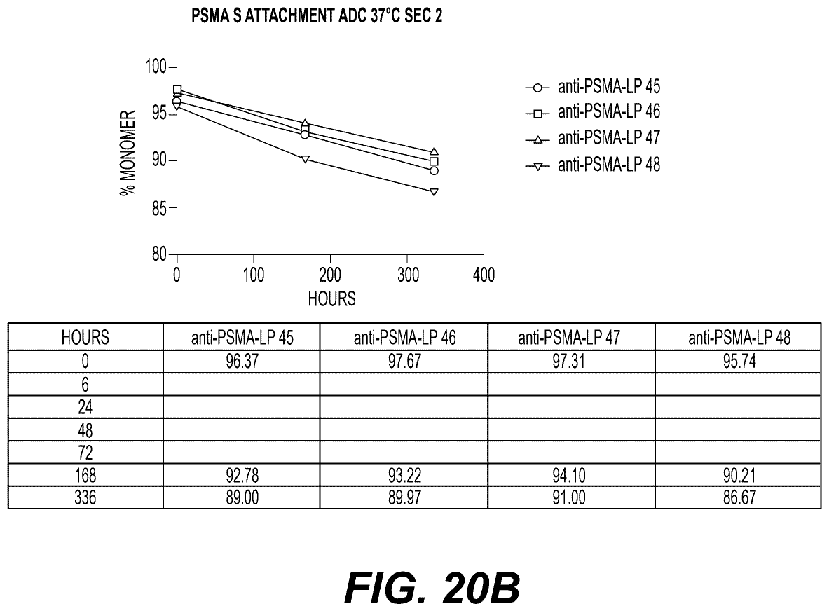

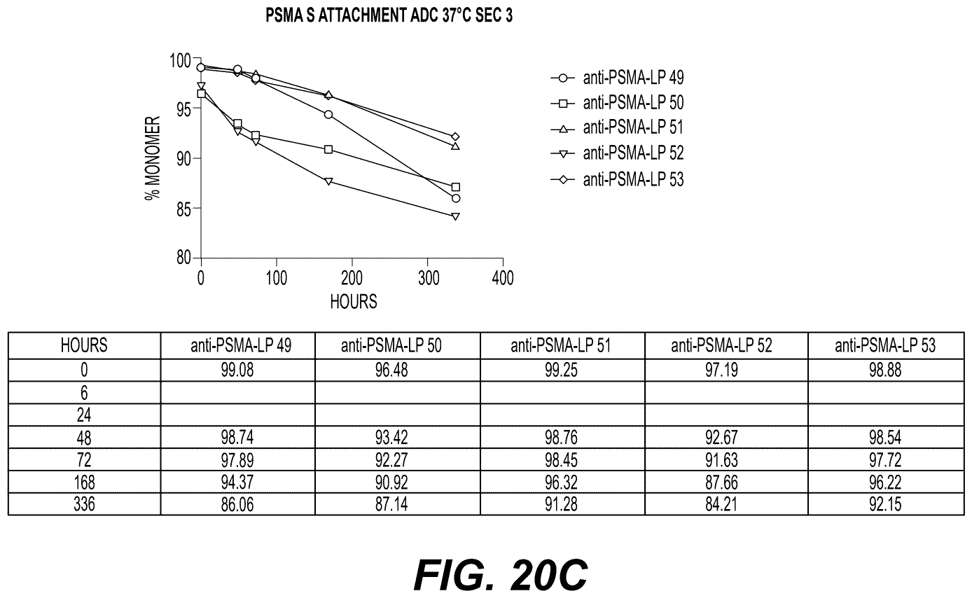

As shown in FIG. 17, ADC-LP3 showed the greatest percent change in DAR over time compared to the other linker-payload options tested. The buffer stability results for S-attached linkers are shown in FIGS. 18-20 and Table 23.

TABLE 23
S-Attachment ADC Buffer Stability Results

| | | | Free Payload |
|---|---|---|---|
| | % Monomer | % DAR change | at T = 14 |
| | T = 14 days | (T14/T0) | (ng/mL, |
| Anti-PSMA-LPₓ | (SEC-HPLC) | (HIC-HPLC) | LC-MS) |
| LP3 | 95.56 (7 day) | 31.81 | 7746.8 |
| LP39 | 92.55 | 81.50 | 737.4 |
| LP42 | 87.74 | 87.21 | 952.6 |
| LP43 | 91.85 | 84.52 | 2101.9 |
| LP44 | 88.50 | 89.37 | 2627.4 |
| LP45 | 89.00 | 78.48 | 2734.6 |
| LP46 | 89.97 | 75.58 | 5876.9 |
| LP47 | 91.00 | 86.58 | 3638.6 |
| LP48 | 86.67 | 56.25 | 7437.4 |
| LP49 | 86.06 | 54.39 | 4207.2 |
| LP50 | 87.14 | 37.37 | 14150.0 |
| LP51 | 91.28 | 76.12 | 3691.8 |
| LP52 | 84.21 | 31.83 | 11250.7 |
| LP53 | 92.15 | 73.53 | 1760.0 |

### 1.2 N-Attached Linkers

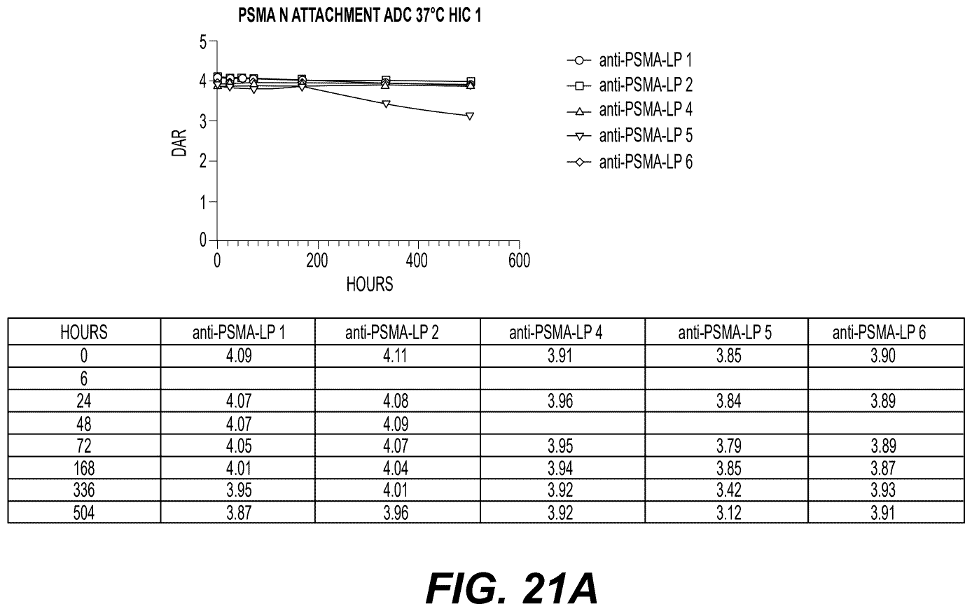

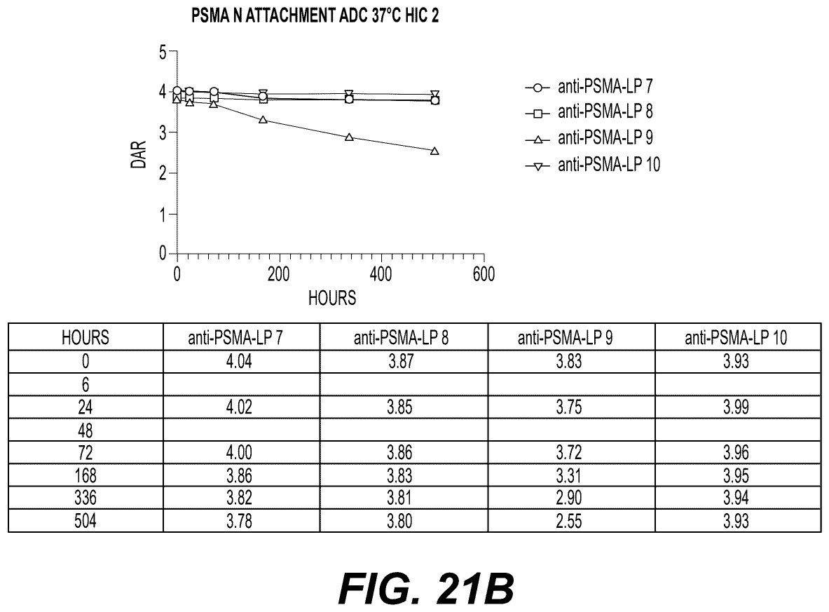

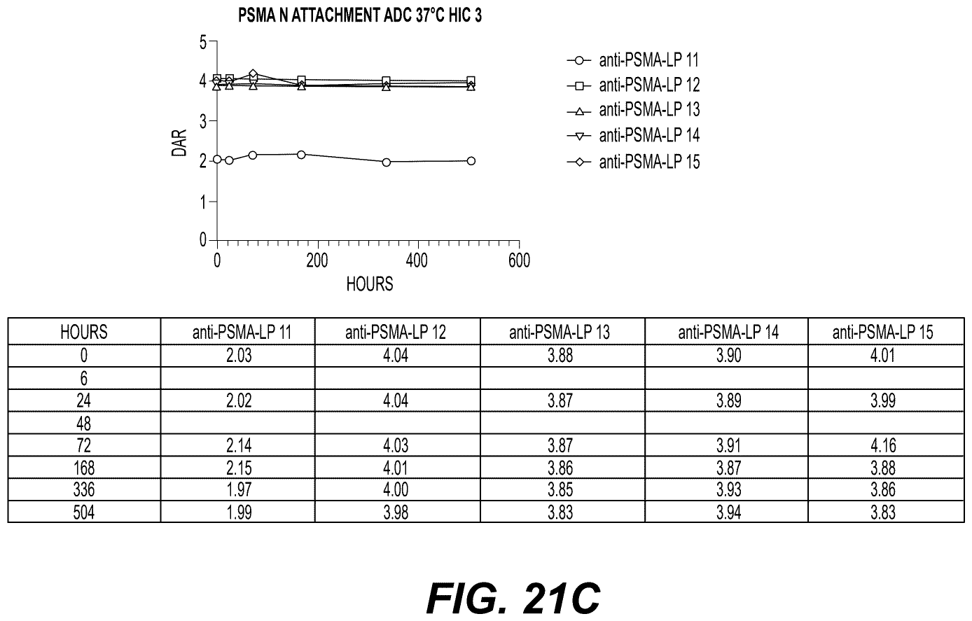

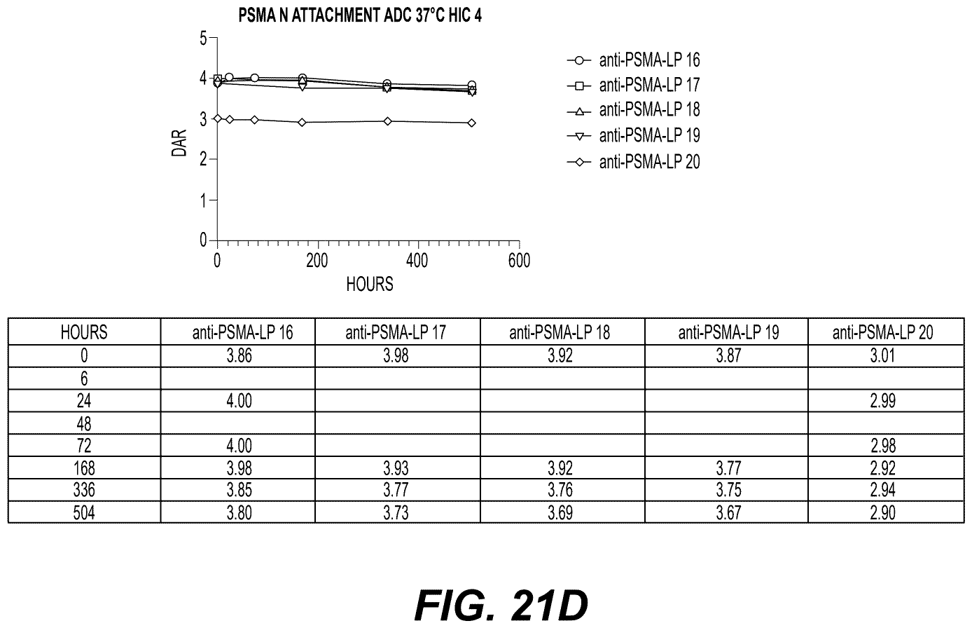

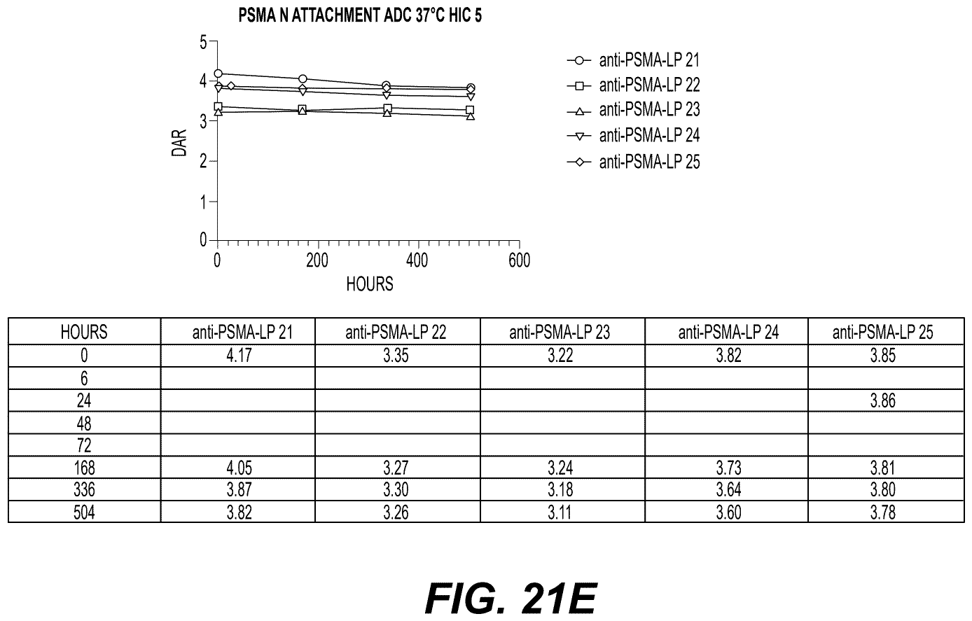

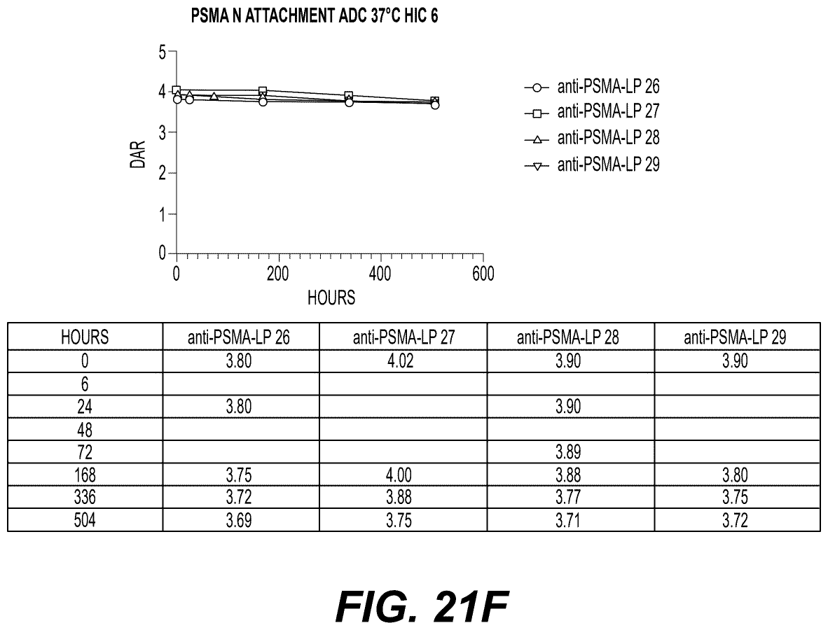

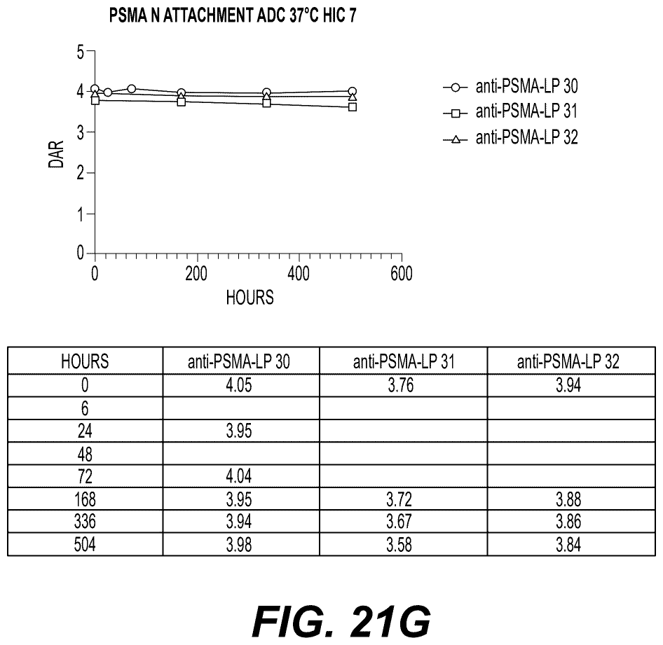

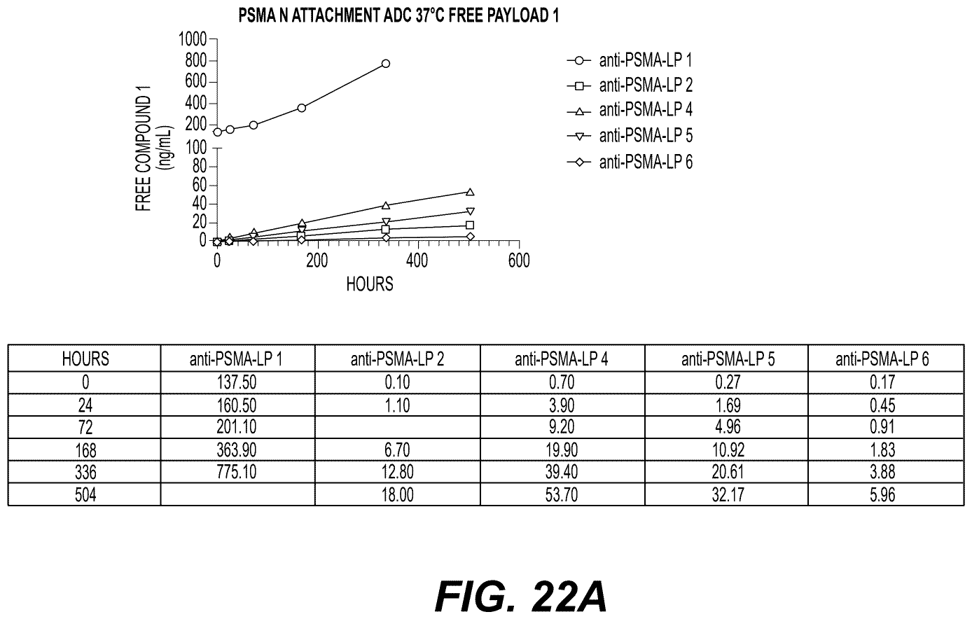

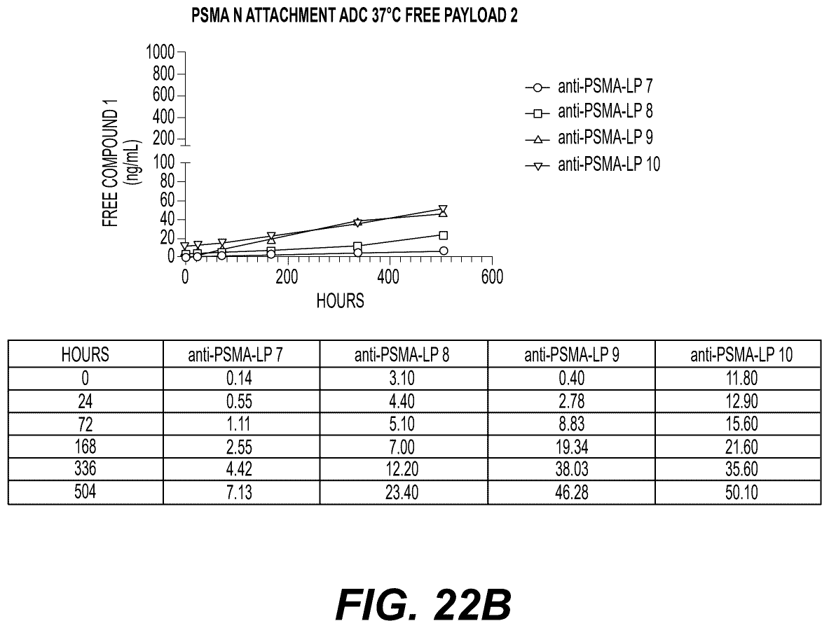

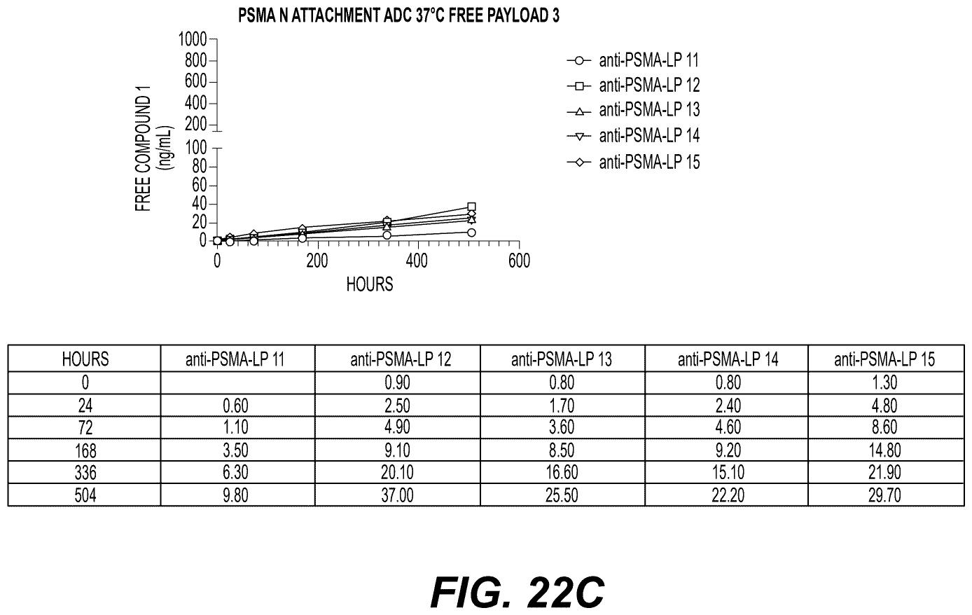

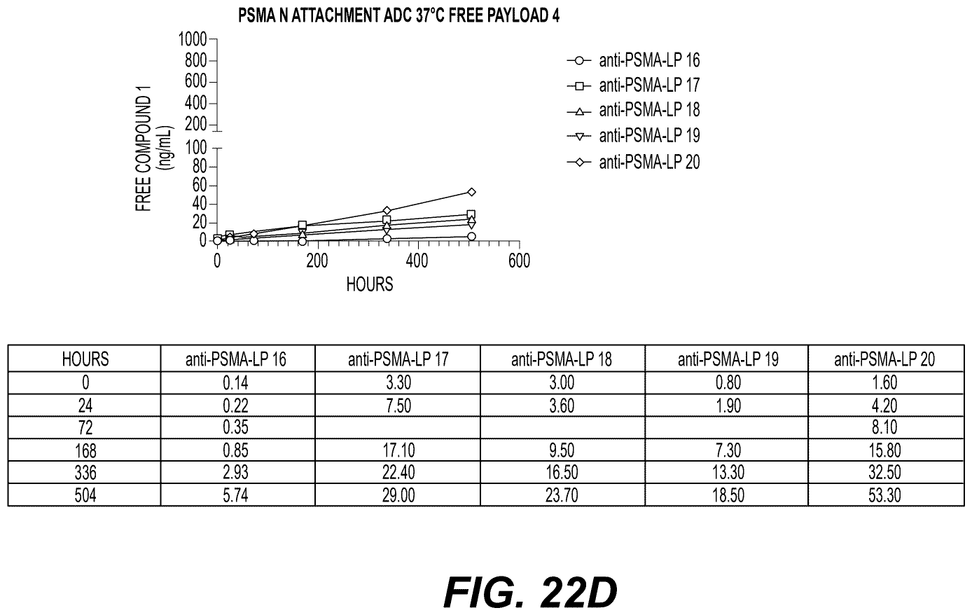

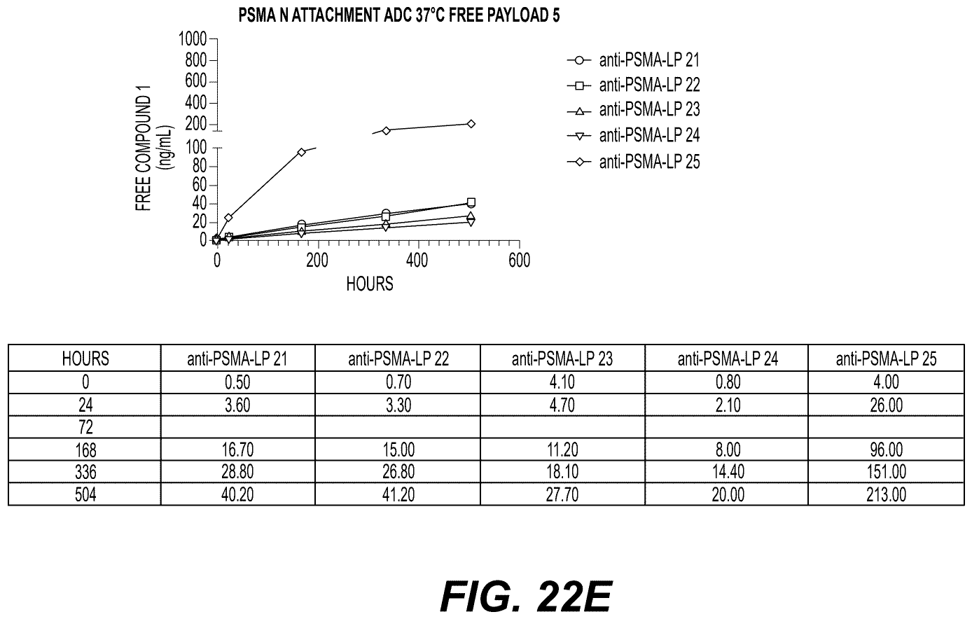

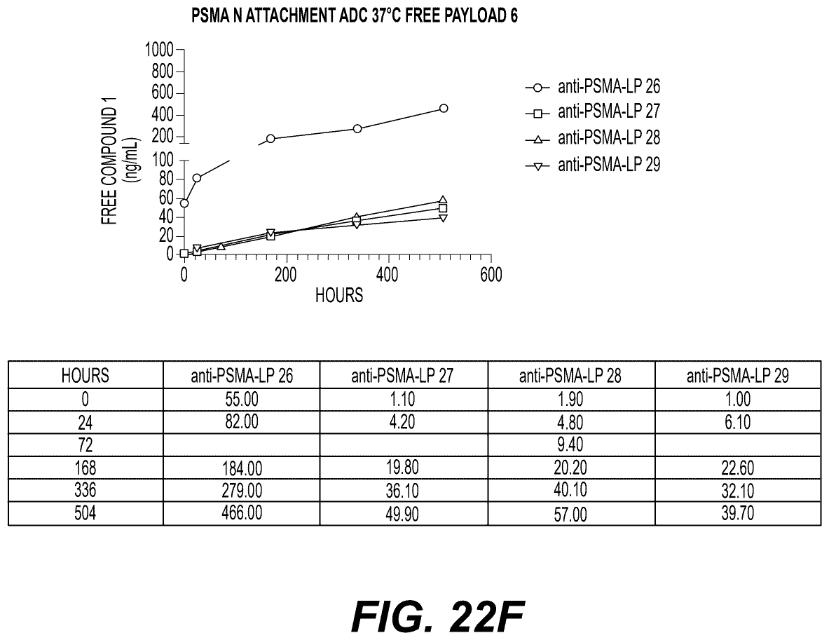

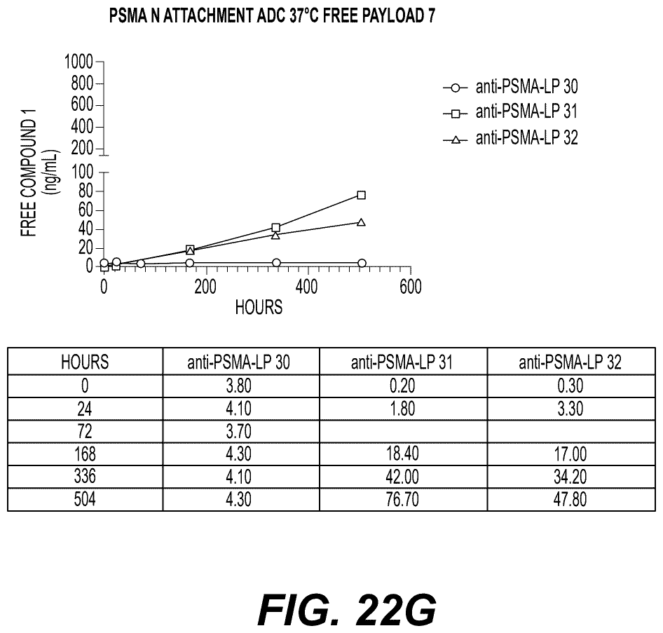

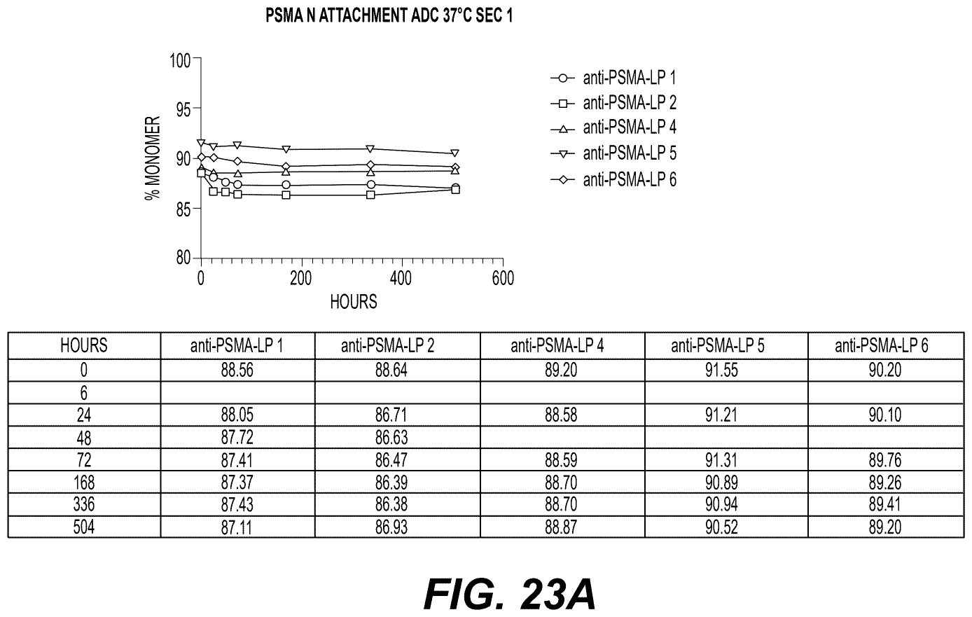

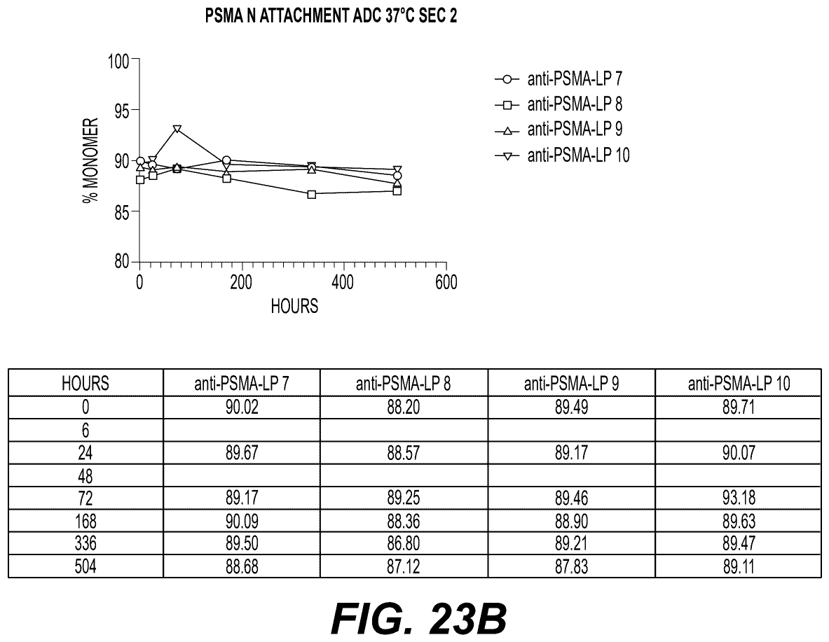

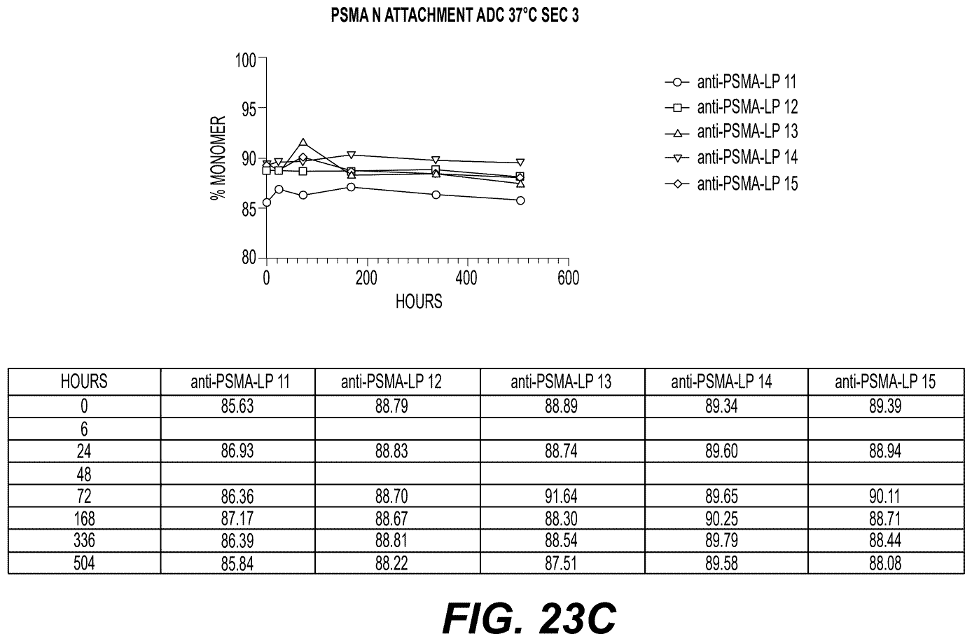

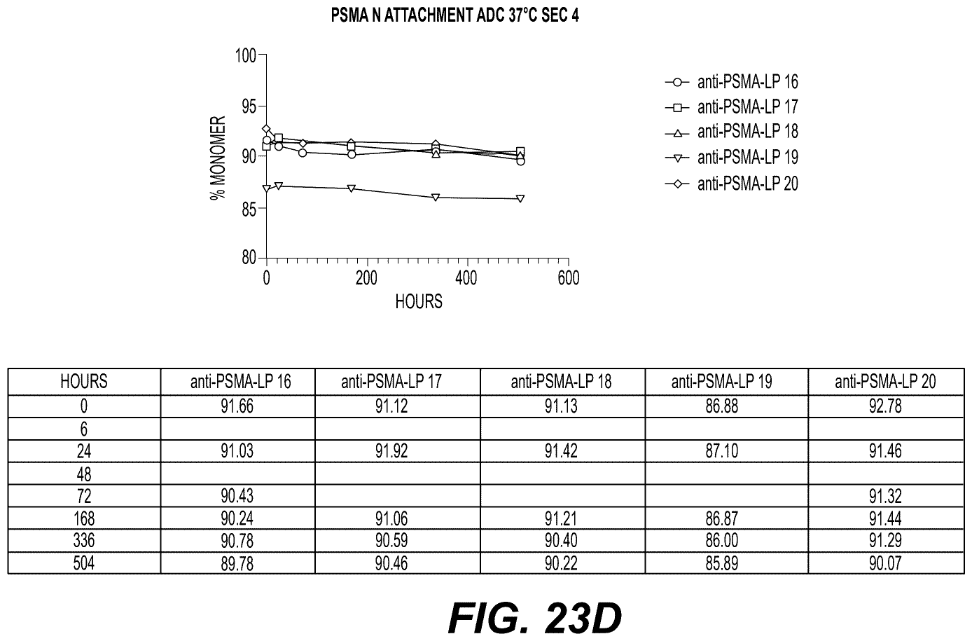

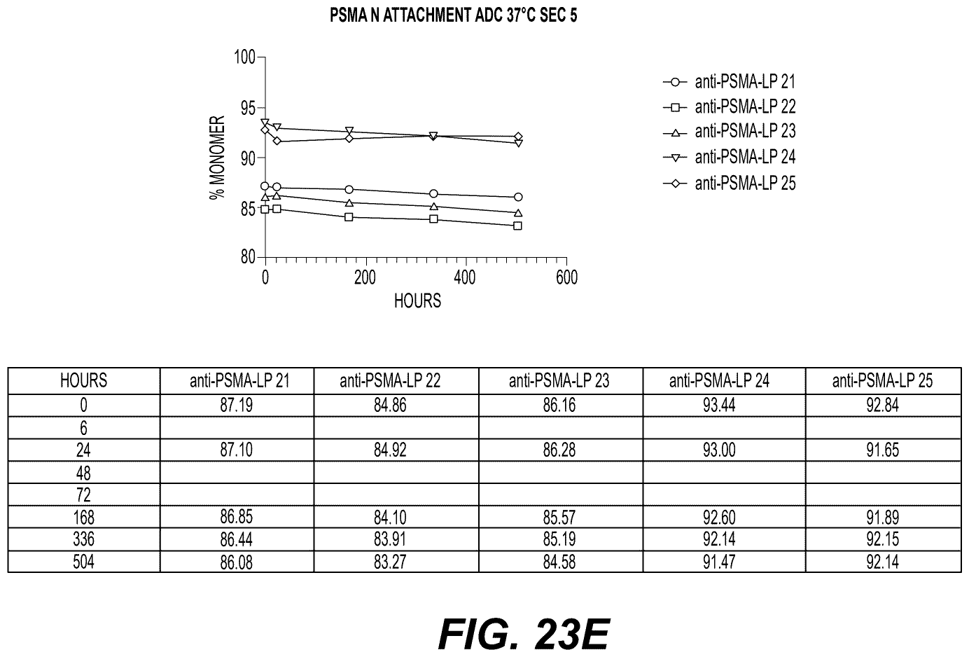

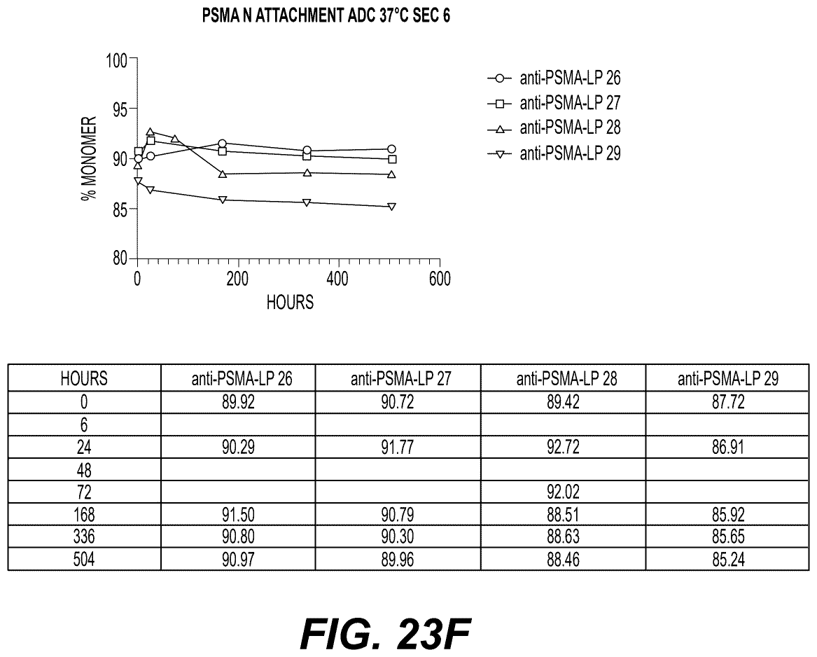

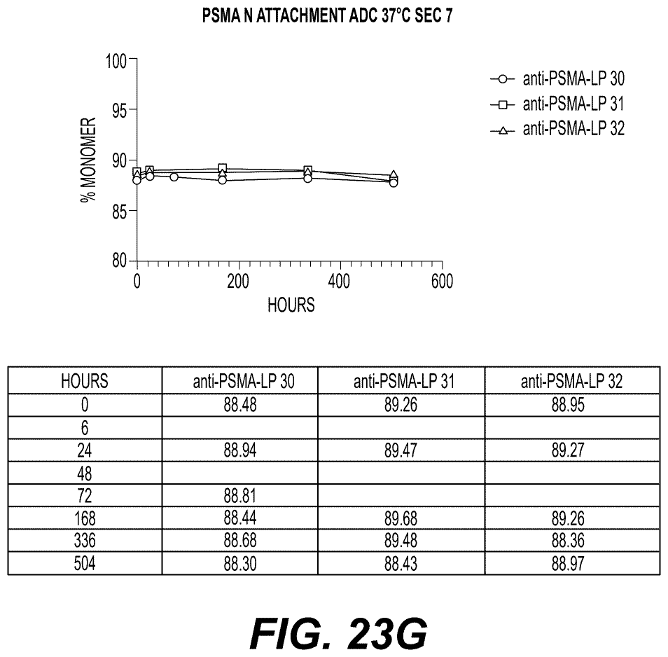

The buffer stability of N-attached linkers was tested as described above. The buffer stability results for N-attached linkers are shown in FIGS. 21-23 and Table 24.

TABLE 24
N-Attachment ADC buffer stability results

| | | | Free Payload |
|---|---|---|---|
| | % Monomer | % DAR change | at T = 21 |
| | T = 21 days | (T21/T0) | (ng/mL, |
| Anti-PSMA-LPₓ | (SEC-HPLC) | (HIC-HPLC) | LC-MS) |
| LP1 | 87.11 | 94.62 | 775.10 (T = 14) |
| LP2 | 86.93 | 96.84 | 18.0 |
| LP4 | 88.87 | 100.26 | 53.7 |
| LP5 | 90.22 | 81.04 | 32.17 |
| LP6 | 89.20 | 100.26 | 5.96 |
| LP7 | 93.56 | 88.68 | 7.13 |
| LP8 | 87.12 | 98.18 | 23.4 |
| LP9 | 87.83 | 66.58 | 46.28 |
| LP10 | 89.11 | 100.00 | 50.1 |
| LP11 | 85.84 | 98.03 | 9.8 |
| LP12 | 88.21 | 98.51 | 37 |
| LP13 | 87.51 | 98.71 | 25.5 |
| LP14 | 89.58 | 101.03 | 22.2 |
| LP15 | 88.08 | 95.51 | 29.7 |
| LP16 | 89.78 | 98.45 | 5.74 |
| LP17 | 90.46 | 93.72 | 29.0 |
| LP18 | 90.22 | 94.13 | 23.7 |
| LP19 | 85.88 | 94.83 | 18.5 |
| LP20 | 89.14 | 100.51 | 53.3 |
| LP21 | 86.07 | 91.61 | 40.2 |
| LP22 | 83.26 | 97.31 | 41.2 |
| LP23 | 84.59 | 96.58 | 27.7 |
| LP24 | 91.47 | 94.24 | 20.0 |
| LP25 | 92.14 | 98.18 | 213 |
| LP26 | 90.97 | 97.11 | 466 |
| LP27 | 89.95 | 93.28 | 49.9 |
| LP28 | 88.46 | 95.13 | 57 |
| LP29 | 85.24 | 95.38 | 39.7 |
| LP30 | 88.30 | 98.52 | 4.3 |
| LP31 | 88.43 | 95.21 | 76.7 |
| LP32 | 88.97 | 97.46 | 47.8 |

### 2 ADC Plasma Stability

### 2.1 Sample Preparation

Plasma stability samples were prepared by diluting J591-Compound 1 ADCs to 0.3 mg/mL or 0.4 mg/mL in mouse plasma (BioIVT, BALB/C mouse plasma prepared using 3.8% sodium citrate as anticoagulant) and aliquoted into 0.1 mL or 0.2 mL time point samples. Samples were placed into 37° C. incubator and aliquots were removed at 0, 2, 6, 24, 48, 72, 168 and 240 hours and frozen at −80° C. immediately after removal.

### 2.2 Analysis of ADC Structure and BAR by LC-MS

Magnetic beads (Dynabeads, Invitrogen, cat 65602) were prepared for immunocapture by washing 3 times with PBS, with collection of the magnetic beads with a DynaMag-2 magnet (Invitrogen, 12321D) after each wash. 40 μg of biotin anti-human Fc (Southern Biotech, 9040-08) per 200 μL stock beads was added to the beads and mixed at room temperature for 1 hr. Beads were washed 3 times with PBS, then brought back to original volume in PBS. 100 μL of plasma sample was added to 200 μL anti-human Fc-immobilized magnetic beads, mixed at room temperature for 2 hr, washed 2 times with PBS, and resuspended in 100 μL 2% acetic acid. Beads were mixed at room temperature for 30 min Supernatant was collected and neutralized with 50 μL 1 M ammonium bicarbonate. 1 unit of FabRICATOR enzyme (Genovis, AO-FR1) per μg ADC was added to the eluted/neutralized sample and incubated for 1 hr at room temperature or digested with microwave assisted digestion (Rapid Enzyme Digestion System, Hudson Surface Technologies), 400W, 37° C. for 15 min. 4 μL of 100 mM DTT was added to reduce samples and incubated for 20 min at 37° C. Samples were transferred to LC sample vials and analyzed by LC-MS on a Waters Synapt G2 fitted with a Waters Acquity UPLC. DAR was calculated as:

$$DAR = [ ( DPA cLc / ( DPA cLC + DPA ucLC ) ) + ( DPA cHC / ( DPA cHC + CPA ucHC ) ) ] \times 2$$

DPA=deconvoluted peak area
cLC=conjugated light chain at LCcys80
ucLC=unconjugated light chain
cHC=conjugated heavy chain at HCcys118
ucHC=unconjugated heavy chain

Percent change in DAR was calculated as:

$$100 - [ ( DAR at x time / starting DAR ) \times 100 ]$$

LC-MS method parameters are shown in Tables 25 and 26 below.

TABLE 25
LC-MS method parameters: Instrumentation
Materials:

| | |
|---|---|
| UPLC | Waters Acquity UPLC |
| MS (Mass Spectrometer) | Waters Synapt G2 Q-TOF MS |
| Column | Waters BioResolve RP Mab polyphenol 450 Å, 2.7 μm, 4.6 × 50 mm, |
| | part no. 186008953 |
| MPA | 0.1% Formic Acid (FA) in Water |
| MPB | 0.1% FA in ACN |
| Weak needle wash | Water: ACN = 97:3 (v:v) |
| Strong needle wash | Water: ACN = 20:80 (v:v) |
| Seal wash | Water: ACN = 90:10 (v:v) |

TABLE 26
LC-MS method parameters

LC method

| | |
|---|---|
| Column temperature | 65 °C. |
| Sample temperature | 10 °C. |
| Injection volume | 5 μL |

25 min Reduced method

| | Time | Flow | | |
|---|---|---|---|---|
| | (min) | (mL/min) | % A | % B |
| Gradients | 0.0 | 0.5 | 75 | 25 |
| (Reduced mAb) | 3.0 | 0.5 | 75 | 25 |
| | 18.0 | 0.5 | 60 | 40 |
| | 19.0 | 0.5 | 10 | 90 |
| | 22.0 | 0.5 | 10 | 90 |
| | 22.5 | 0.5 | 75 | 25 |
| | 25.0 | 0.5 | 75 | 25 |

MS method

| | | |
|---|---|---|
| MS tune file ES+ | Capillary (kV) | 2.50 |
| | Sample Cone | 30 |
| | Extraction Cone | 4.0 |
| | Source Temp | 80 |
| | Desolvation Temp | 600 |
| | Cone Gas (L/h) | 30 |
| | Desolvation Gas (L/h) | 1000 |

| Fluidics | Flow State: LC | |
|---|---|---|
| MS method | | |
| Reduced mab_500_4000 | T of MS (for reduced mAb) | |
| Method Events | Initial Condition Flow State | Waste Sample |
| | 4.00 Flow State | LC Sample |
| | Acquisition Times | 0-24 min |
| | Source | ES |
| | Polarity | Positive |
| | Analyser Mode | Sensitivity |
| | Dynamic Range | Normal |
| | Sensitivity | Normal |
| | TOF MS | |
| | Low Mass | 500 Da |
| | High Mass | 4000 Da |
| | Scan Time | 1 000 sec |
| | Data Format | Continuum |
| | Trap CE Control | Default |
| | Transfer CE Control | Default |
| Data process method | | |
| MaxEnt 1 | Reduced Mass | |
| Ranges | 20,000-35,000 Da | |
| Resolution | 0.5 Da/channel | |
| Uniform Gaussian - Width | 0.5 Da | |
| at half height | | |
| Simulated Isotope Pattern | 0.5 Da | |
| Spectrometer Blur | | |

| | |
|---|---|
| Width | Left 33% Right 33% |
| Minimum intensity ratios | Iterate to convergence |

### 2.3 Analysis of Free Payload by Quantitative LC-MS

Compound 1 and D6-Compound 1 internal standard (IS) powdered reagents were dissolved in LC-MS-grade water. Compound 1 reference standards (standard curve), and QC standards (QC-low, QC-med, and QC-hi) were prepared in mouse plasma, and internal standard for injection was prepared in water. 10 μL of IS was added to each of the standard curve samples, QC controls, and stability samples, and all were prepared using solid-phase extraction (SPE). For SPE, a solid-phase extraction plate (Waters, 186001828A) was washed with 100 μL water using vacuum. 100 μL of each sample was applied to the plate and drawn through using vacuum. Wells were washed with 5% methanol in water using vacuum. Samples were eluted into a clean collection plate with 25 μL acetonitrile:methanol (60:40). The collection plate was briefly centrifuged to collect all eluted material to the bottom of the collection wells, then 100 μL water was added to each well. Samples were then used for quantitative LC-MS analysis. 5 μL or 10 μL of each eluted sample was injected for LC-MS analysis. Quantitation was done using TargetLynx (Waters). LOQ for Compound 1 was 0.01 ng/mL and 0.05 ng/mL for Rp-monophosphate-Compound 1 and Sp-monophosphate-Compound 1.

LC-MS parameters are shown in Tables 27 and 28 below.

TABLE 27
LC-MS Instrumentation

| | |
|---|---|
| UPLC | Waters Acquity I class plus UPLC |
| MS | Waters Xevo TQ-XS Triple Quadrupole MS |
| Column | Waters Acquity UPLC BEH C18 |
| | 2.1 × 50 mm, 130 Å, 1.7 μm, Part No. 186002350 |
| Mobile phase A | 5 mM ammonium bicarbonate in H₂O |
| Mobile phase BB | 5 mM ammonium bicarbonate in MeOH |
| Injection solvent | H₂O |

TABLE 28
LC-MS method

| Sample preparation | | | | | | |
|---|---|---|---|---|---|---|
| Standards in solution | Serial dilution with injection solvent | | | | | |
| Samples in matrix | Solid phase extraction (SPE) on Waters Oasis HLB 96-well μElution | | | | | |
| | Plates (186001828BA) | | | | | |
| | Wash: 5% MeOH, Elute: ACN/MeOH = 60/40 | | | | | |
| Inlet method | Compound 1-NH4 20210927_20% B | | | | | |
| Column temperature | 40° C. | | | | | |
| Sample temperature | 15° C. | | | | | |
| Injection volume | 5 μL | | | | | |
| | Time | Flow | | | | |
| Gradient | (min) | (mL/m) | % A | % B | | Curve |
| | 0.0 | 0.5 | 95.0 | 5.0 | | 6 |
| | 2.0 | 0.5 | 80.0 | 20.0 | | 6 |
| | 2.1 | 0.5 | 10.0 | 90.0 | | 6 |
| | 4.0 | 0.5 | 10.0 | 90.0 | | 6 |
| | 4.1 | 0.5 | 95.0 | 5.0 | | 6 |
| | 6.0 | 0.5 | 95.0 | 5.0 | | 6 |
| MRM method | MRM Compound 1 and mP-Compound 1 with IS 20210927 | | | | | |
| | | | | Cone | Colli. | |
| | Mass | Parents | Daughters | Volt. | Eng. | Dwell |
| Compound 1-NH4 | | 747.0700 | 171.0000 | 90 | 72 | 0.025 |
| | | 747.0700 | 187.9700 | 90 | 64 | 0.025 |
| D6 Compound 1IS | | 753.2700 | 194.1100 | 100 | 56 | 0.025 |

| Event | Divert to the waste at 2.2 min | |
|---|---|---|
| MS tune file (ES+) | Tune Compound 1-NH4 20210901 | |
| | Capillary (kV) | 2.1 |
| | Sample Cone (V) | 93 |
| | Desolvation Temp (° C.) | 600 |
| | Desolvation Gas (L/h) | 1000 |
| | Cone Gas (L/h) | 150 |
| | Nebuliser (Bar) | 7.0 |
| | LM Resolution 1 | 2.79 |
| | HM Resolution 1 | 14.29 |
| | Ion Energy 1 | −0.8 |
| | LM Resolution 2 | 2.46 |
| | HM Resolution 2 | 14.45 |
| | Ion Energy 2 | 0.2 |
| | Collision gas Flow (mL/min) | 0.16 |
| | Collision | 4 |
| TargetLynx method | TargetLynx Compound 1 and mP-Compound 1 with IS 20220105 | |
| | Retention Time (min) | |
| | Compound 1 | 1.70 |
| | D6 Compound 1 | 1.70 |

### 2.4 Results

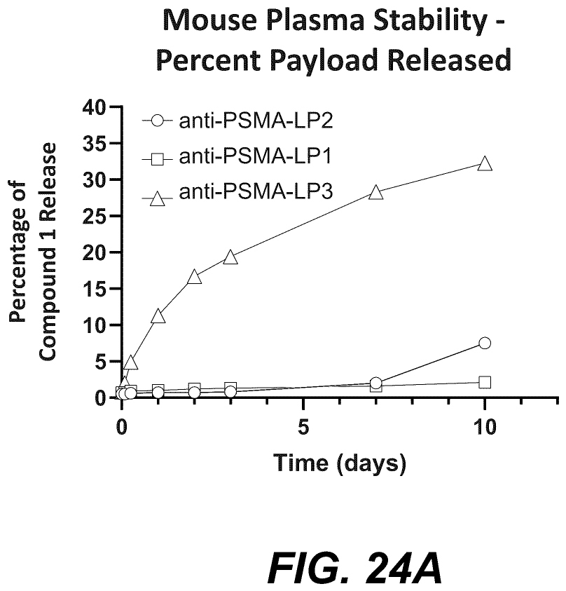

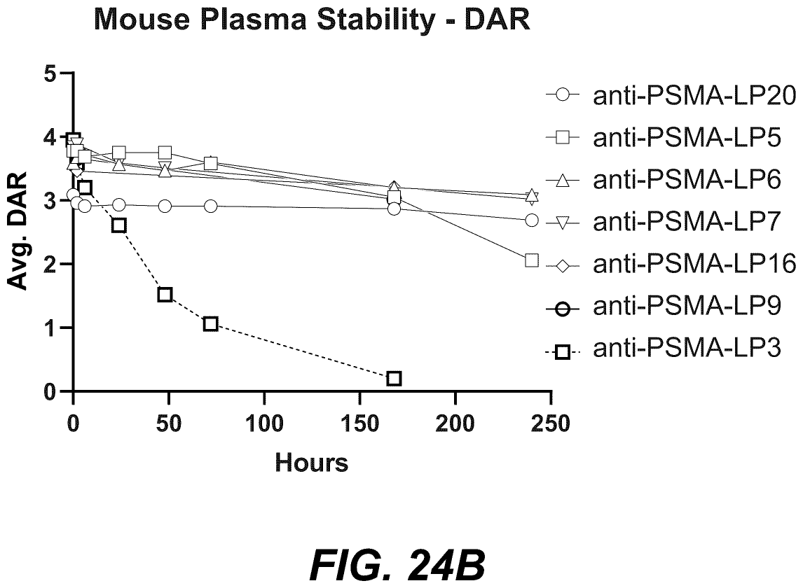

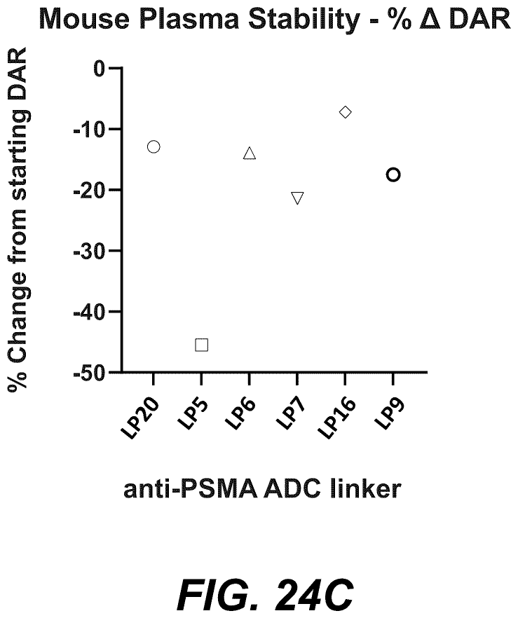

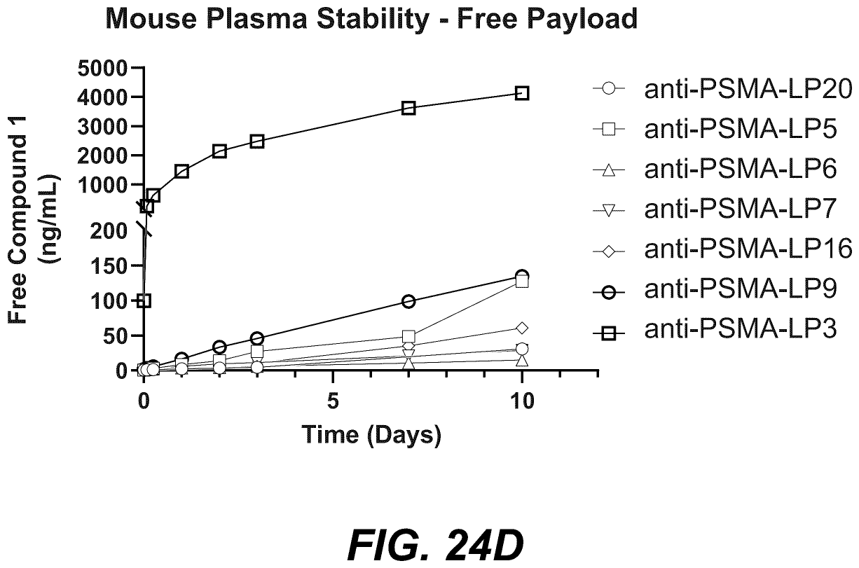

Plasma stability results are shown in FIG. 24. Percent of payload release over time was lower in serum from mice treated with anti-PSMA-LP2 or anti-PSMA LP1 as compared to LP3 (FIG. 24A). The decrease in average DAR over time was minimal in N-inked ADCs compared to the 5-linked ADC (LP3) (FIGS. 24B and 24C). Treatment with N-inked ADCs resulted in significantly less free Compound 1 in serum over time (FIG. 24D).

### 2.4.1 Stability of S-Linked Anti-PSMA Compound 1 ADCs

Stability of anti-PSMA ADCs linked via the thiophosphate bond in Compound 1 (“5-linked ADCs”) were examined in mouse plasma at 0.4 mg/mL. The linker-payload structures are shown in Tables 15 and 16. These were conjugated to humanized anti-human PSMA deJ591 via either unpaired cysteines generated through partial reduction of interchain disulfide bonds (random DAR4) or through LC cys80 (RESPECT-L) and LC A118C (RESPECT-L DAR4).

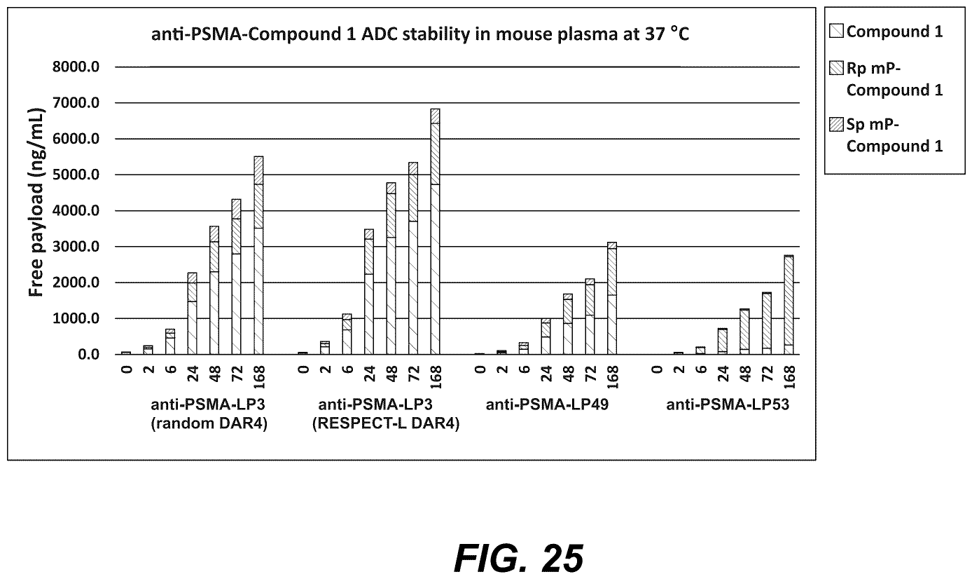

Analysis of released payload was performed via quantitative LC-MS using a Waters TQ-XS triple-quad mass spectrometer after SPE extraction of plasma samples. The results of this are shown in FIG. 25.

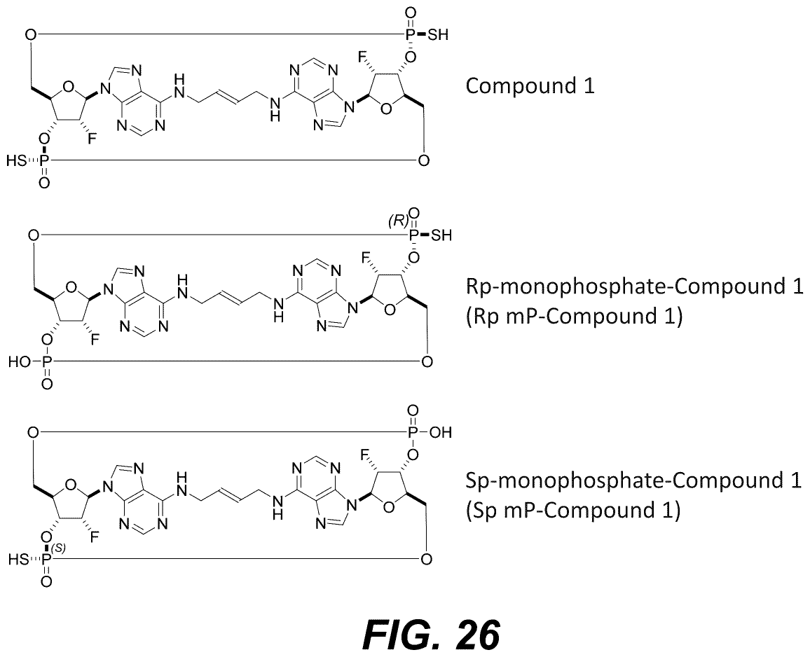

Both random and RESPECT-L conjugated LP3 ADCs were found to be generally unstable in mouse plasma, as total conjugated payload in 0.4 mg/mL ADC sample at DAR4 is 7957 ng/mL. It was also found that three molecular forms of the payload were released—Compound 1, Rp-monophosphate-Compound 1, and Sp-monophosphate-Compound 1. These forms likely result from S—P or C—S bond cleavage in the linker-payload during metabolism, as well as potentially from isomerization of the thiophosphate bond during or after release. The structures of the three released payload forms are shown in FIG. 26.

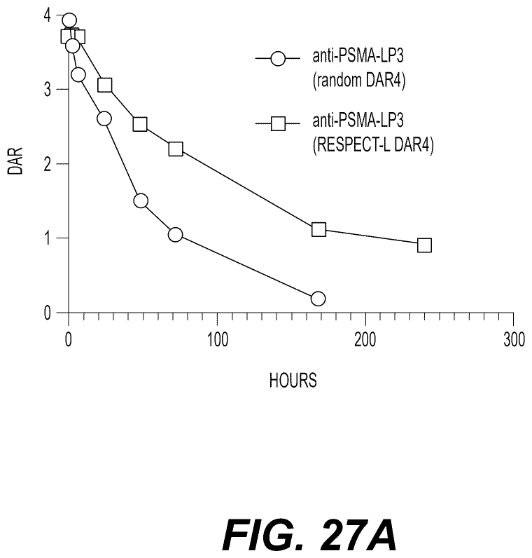

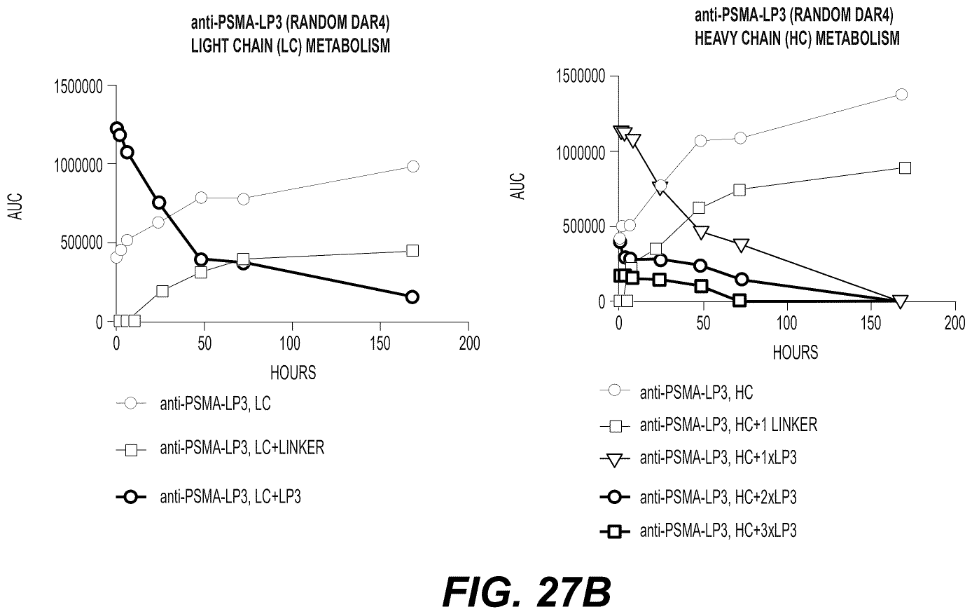

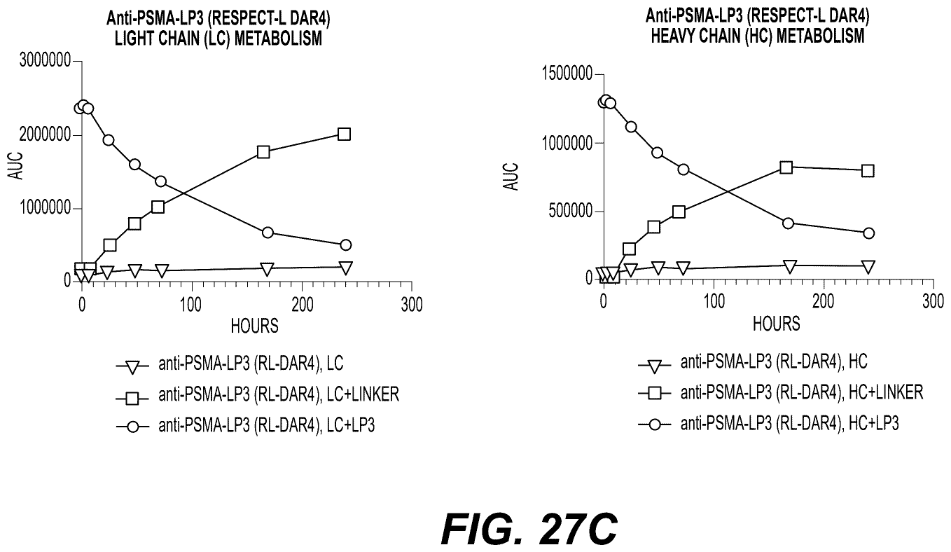

Anti-PSMA ADC LP3 stability samples were also analyzed by hybrid LC-MS following immunocapture of the ADCs from mouse plasma. The results of this are shown in FIG. 27. AUC values are AUC for deconvoluted mass spectra, corresponding to each indicated structure. Values are normalized for total AUC of all species at TO.

Both ADCs demonstrated significant payload metabolism and release from linker (increase in LC+linker and HC+linker). For randomly conjugated ADC, only LC+1 linker and HC+1 linker was observed; it is likely abundance of other multiply-conjugated forms (e.g., HC+1 linker+1 LP3, HC+2 linker, HC+2 linker+1 LP3, etc.) were too low in abundance to be reliably detected and quantitated. For RESPECT-L ADC, metabolism was almost exclusively due to payload release from linker and not release of linker-payload from antibody due to retro-Michael deconjugation, indicated by the lack of increase of unconjugated LC and HC. For the randomly conjugated LP3 ADC, both metabolism of linker payload, resulting in release of free payload, and retro-Michael deconjugation of linker-payload, resulting in increase in free LC and HC, were observed. This likely explains the lower rate of DAR loss for the RESPECT-L conjugated form of the LP3 ADC.

### 2.4.2 Stability of N-Linked Anti-PSMA Compound 1 ADCs

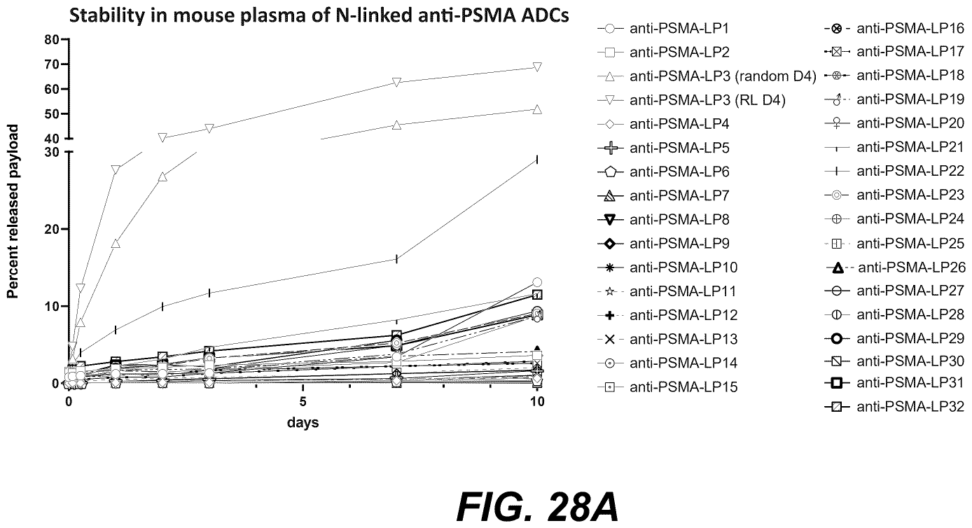

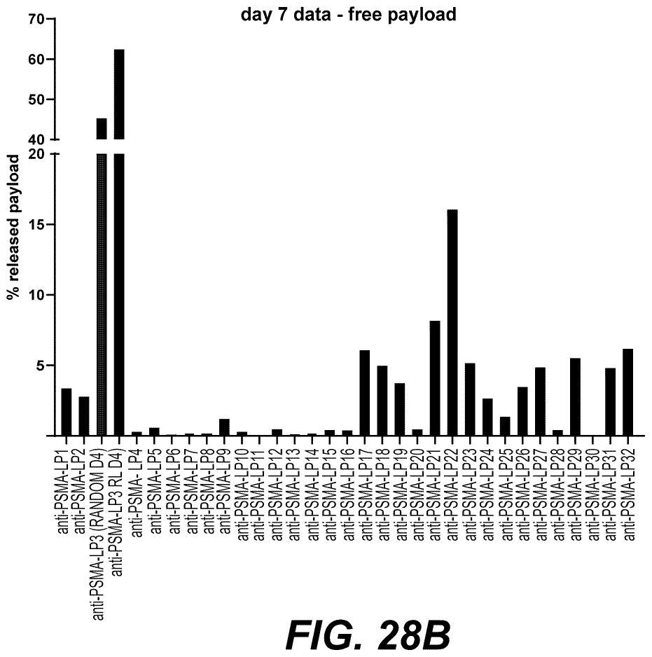

Stability of anti-PSMA ADCs linked via the bridge nitrogen in Compound 1 (“N-linked ADCs”) were examined in mouse plasma at 0.3 mg/mL or 0.4 mg/mL. These ADCs were prepared via RESPECT-L DAR4 conjugation to LC cys80 and HC A118C unpaired cysteines. Free released payload was assessed via quantitative LC-MS using a Waters TQ-XS triple-quad mass spectrometer after SPE extraction of plasma samples. To allow for comparison between ADCs, free payload is expressed as the percentage of total conjugated payload at time TO. These results are shown in FIG. 28.

Anti-PSMA ADC LP3 (random and RESPECT-L) is included in plotted data (total of Compound 1, Rp-monophosphate Compound 1, and Sp-monophosphate Compound 1). In general, all N-linked anti-PSMA Compound 1 ADCs were more stable than S-linked ADCs. In addition, Compound 1 was the only detected payload released; no monophosphate forms were released. Anti-PSMA LP22 (mcA(NMe)AN-Unit 9-SN-Compound 1) demonstrated significantly higher payload release than other spacer, cleavage site, or second spacer variants. In general, L-F₂Pro and Bz second spacers appeared to be the least stable in mouse plasma (highest free payload release), followed by L-Pro, then MEC. Additionally, mc spacer between the maleimide and cleavage site appeared to lead to lower payload release levels regardless of the second spacer structure (compared with C2 and C2-PEG₂ spacers).

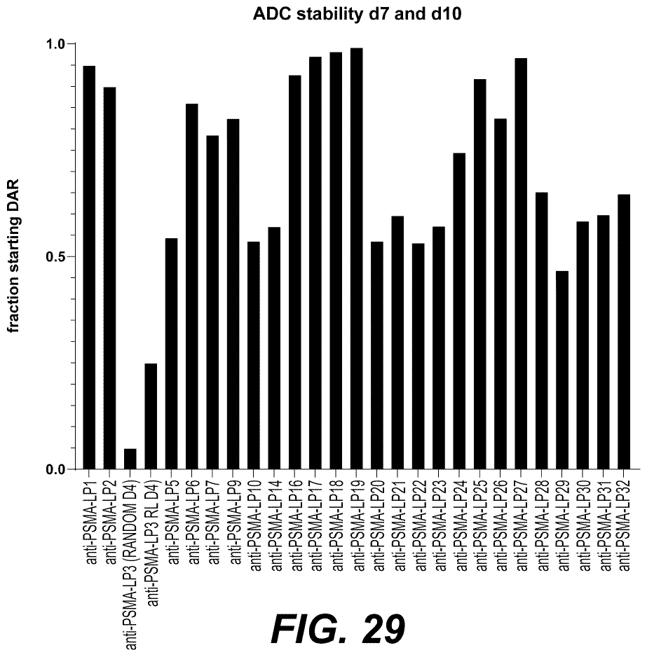

Anti-PSMA Compound 1 ADC stability samples were also analyzed by hybrid LC-MS following immunocapture of the ADCs from mouse plasma. Data was plotted as percent of remaining DAR relative to DAR at day 0, in order to allow for comparison of all ADCs. Anti-PSMA ADC LP3 (random and RESPECT-L) is included in plotted data. Day 7 or 10 data (e.g., showing the last time point of stability study) is plotted. These results are shown in FIG. 29.

All N-linked ADCs demonstrated better overall DAR retention than S-linked ADCs. Furthermore, the analyses indicated that loss of DAR for N-linked ADCs is due to retro-Michael release of linker-payload rather than by metabolism of payload from linker (comparing DAR to free payload), as was observed for anti-PSMA ADC LP3. ADCs with caproyl spacers were variable in stability, while C2 and C2PEG₂ spacer variants were generally very stable, in terms of linker-payload release, likely due to maleimide hydrolysis post conjugation.

### Example 10. N-Linker Attachment Demonstrates Efficacy In Vitro and In Vivo

### 1 hIFN6 Release in C4-2+THP1 Co-Culture

C4-2 cells were seeded in 96-well plates at 1×10⁵ cells/well in 100 μL of cell culture medium (complete RPMI, 10% FBS), then 20 μL of ADC reagents were added at a semi-log serial dilution from 0.03-1000 nM, mixed well, and incubated at 37° C., 5% CO₂ for 10 minutes. 1×10⁵ cells/well of THP1 cells in 80 μL medium were added to the well, and the cell plate was incubated at 37° C., 5% CO₂ for 6 hours. The plate was centrifuged at 1500 rpm for 5 minutes, and supernatant was collected and stored at −80° C. for hIFNβ measurement.

hIFNβ was measured using human IFNβ U-PLEX (MSD, Cat #K151VIK-2), following the manufacturer's instruction. The dynamic range of hIFNβ is 3.1-100,000 μg/ml.

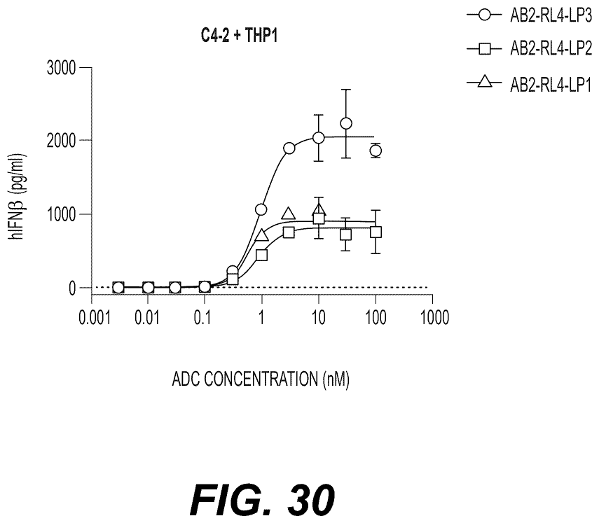

As shown in FIG. 30, AB2-RL4-LP3 induces more IFNβ production than AB2-RL4-LP1 and AB2-RL4-LP2 in the C4-2/THP1 co-culture assay. AB2-RL4-LP1 and AB2-RL4-LP2 showed similar IFNβ induction in the C4-2/THP1 co-culture assay.

### 2 In Vivo Efficacy in C4-2 Model

CB17 SCID male mice were used for this study. 10×10⁶ of C4-2 cells suspended in 100 μL of 1×PBS containing 50% Matrigel was implanted subcutaneously in the right flank of each mouse and tumor growth was monitored. The mice were randomized into five groups (10 mice per group) and received treatment as indicated below when tumor size reached about 150 mm³. The mouse was treated with a single intravenous dose as indicated, and the tumor size and body weight were monitored for 35 days.
Group 1, Vehicle (PBS) (IV), single dose
Group 2, 8 mg/kg Anti-PSMA-LP1 (IV), single dose
Group 3, 4 mg/kg Anti-PSMA-LP1 (IV), single dose
Group 4, 8 mg/kg Anti-PSMA-LP2 (IV), single dose
Group 5, 4 mg/kg Anti-PSMA-LP2 (IV), single dose

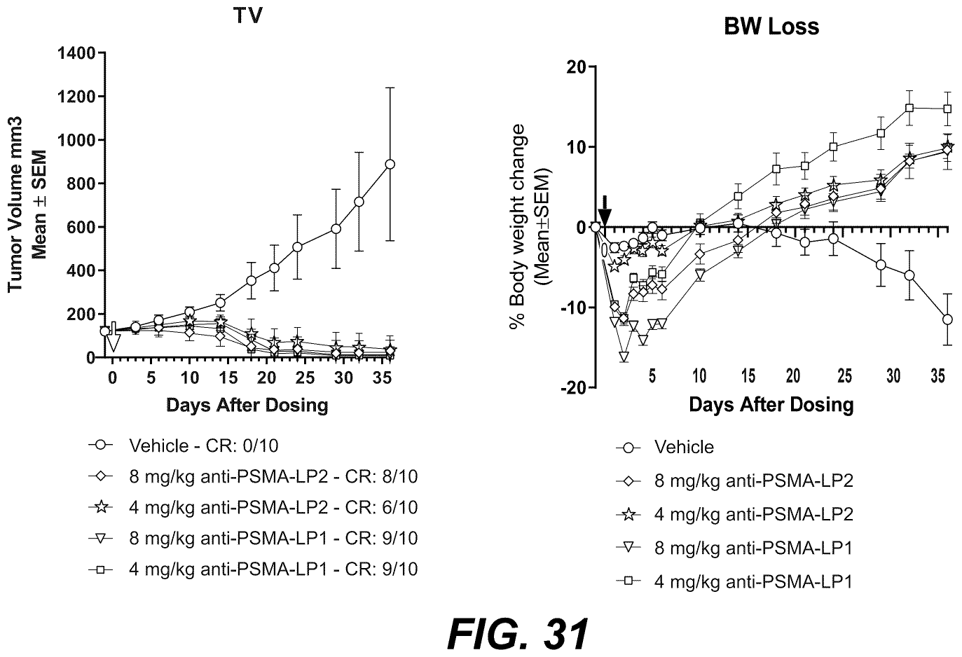

Both anti-PSMA ADC groups at 4 mg/kg and 8 mg/kg showed tumor regression (FIG. 31). A transient body weight loss (<20%) was observed for both ADC groups with eventual recovery. The group treated with ADCs comprising LP1 showed less body weight loss compared with the group treated with ADCs comprising LP2. The vehicle group started body weight loss after 25 days when mice were sick, while the other four treatment groups started to gain weight when the tumor growth was inhibited.

### Example 11. In Vivo Efficacy Study of ADCs in RM1-hPSMAg(Teton) Murine Prostate Cancer Model in C57BL/6 mice

### 1 Summary of Methods

In this study, the efficacy of the SEB-LP3 ADC, AB2-RL4-LP2, AB2-RL4-LP3, and Enzalutamide were evaluated in the treatment of subcutaneous model RM1-hPSMAg in C57BL/6 mice.

RM1-hPSMAg tumor cells were inoculated into male C57BL/6 mice. Treatment was initiated when tumors reached mean tumor volume around 84 mm³. Mice were randomized into ten groups (10 mice per group) as shown below.

| | | | | Dosing | | | |
|---|---|---|---|---|---|---|---|
| | Animal | | | Solution | Dosing | Dosing | Dosing Frequency & |
| Group | Number | Treatment | Dose Level | (mg/ml) | Volume | route | Duration |
| 1 | 10 | Vehicle | — | — | 10 μL/g | i.v. | Single dose (at the |
| | | | | | | | day post |
| | | | | | | | randomization) |
| | | Doxycycline | 2 mg/mouse | 10 | 200 μL/mouse | oral | Two doses (start at |
| | | | | | | | the randomization |
| | | | | | | | day and dosing two |
| | | | | | | | days continuously) |
| 2 | 10 | SEB-LP3 | 1 mg/kg | 0.1 | 10 μL/g | i.v. | Single dose (at the |
| | | ADC | | | | | day post |
| | | | | | | | randomization) |
| | | Doxycycline | 2 mg/mouse | 10 | 200 μL/mouse | oral | Two doses (start at |
| | | | | | | | the randomization |
| | | | | | | | day and dosing two |
| | | | | | | | days continuously) |
| 3 | 10 | AB2-RL4- | 4 mg/kg | 0.4 | 10 μL/g | i.v. | Single dose (at the |
| | | LP1 | | | | | day post |
| | | | | | | | randomization) |
| | | Doxycycline | 2 mg/mouse | 10 | 200 μL/mouse | oral | Two doses (start at |
| | | | | | | | the randomization |
| | | | | | | | day and dosing two |
| | | | | | | | days continuously) |
| 4 | 10 | AB2-RL4- | 1 mg/kg | 0.1 | 10 μL/g | i.v. | Single dose (at the |
| | | LP3 | | | | | day post |
| | | | | | | | randomization) |
| | | Doxycycline | 2 mg/mouse | 10 | 200 μL/mouse | oral | Two doses (start at |
| | | | | | | | the randomization |
| | | | | | | | day and dosing two |
| | | | | | | | days continuously) |
| 5 | 10 | Enzalutamide | 25 mg/kg | 2.5 | 10 μL/g | oral | QD × 21 days (start |
| | | | | | | | dosing at the day |
| | | | | | | | post randomization |
| | | | | | | | and dosing 21 days |
| | | | | | | | continuously) |
| | | Doxycycline | 2 mg/mouse | 10 | 200 μL/mouse | oral | Two doses (start at |
| | | | | | | | the randomization |
| | | | | | | | day and dosing two |
| | | | | | | | days continuously) |
| 6 | 10 | Vehicle | — | — | 10 μL/g | i.v. | Single dose |
| | | | | | | | (at 3 days post |
| | | | | | | | randomization) |
| | | Doxycycline | 2 mg/mouse | 10 | 200 μL/mouse | oral | Four doses (start at |
| | | | | | | | the randomization |
| | | | | | | | day and dosing four |
| | | | | | | | days continuously) |
| 7 | 10 | AB2-RL4- | 4 mg/kg | 0.4 | 10 μL/g | i.v. | Single dose |
| | | LP1 | | | | | (at 3 days post |
| | | | | | | | randomization) |
| | | Doxycycline | 2 mg/mouse | 10 | 200 μL/mouse | oral | Four doses (start at |
| | | | | | | | the randomization |
| | | | | | | | day and dosing four |
| | | | | | | | days continuously) |
| 8 | 10 | AB2-RL4- | 1 mg/kg | 0.1 | 10 μL/g | i.v. | Single dose |
| | | LP3 | | | | | (at 3 days post |
| | | | | | | | randomization) |
| | | Doxycycline | 2 mg/mouse | 10 | 200 μL/mouse | oral | Four doses (start at |
| | | | | | | | the randomization |
| | | | | | | | day and dosing four |
| | | | | | | | days continuously) |
| 9 | 10 | Vehicle | — | — | 10 μL/g | i.v. | Single dose |
| | | | | | | | (at the day post |
| | | | | | | | randomization) |
| 10 | 10 | AB2-RL4- | 1 mg/kg | 0.1 | 10 μL/g | i.v. | Single dose |
| | | LP3 | | | | | (at post |
| | | | | | | | randomization) |

i.v. = intravenous

Body weights and tumor volumes were measured twice weekly after randomization. The tumor inoculation day is denoted as Day 1. Doxycycline treatment was started at the randomization day. Vehicle, SEB-1L13 ADC, AB2-RL4-LP1, and AB2-RL4-LP3 treatment were started the day after randomization.

The mice in groups 1-4 were treated with doxycycline at the randomization day and at the day post-randomization. At the day post-randomization, the mice were dosed with either vehicle or SEB-LP3 ADC, AB2-RL4-LP1, or AB2-RL4-LP3, 4-6 hours after doxycycline treatment.

The mice in groups 6-8 were treated with doxycycline starting at the randomization day and for 4 days continuously. At the fourth administration of doxycycline, the mice were dosed with either vehicle or AB2-RL4-LP1 or AB2-RL4-LP3, 4-6 hours after doxycycline treatment.

The mice in group 5 were treated with doxycycline at the randomization day and at the day post randomization. At the day post randomization, the mice were dosed with Enzultamide 4-6 hours after doxycycline treatment.

The mice in group 9 and group 10 were dosed with either vehicle (group 9) or AB2-RL4-LP3 at the day post randomization.

The mice in groups 2, 3, 4, 7, 8, and 10 were given subcutaneous fluid supplement for three consecutive days, and the supplement was started at 24 hours post-treatment with SEB-LP3 ADC, AB2-RL4-LP1, or AB2-RL4-LP3.

Tumor volumes were measured twice per week in two dimensions using a caliper, and the volume was expressed in mm³ using the formula: V=(L×W×W)/2, where V is tumor volume, L is tumor length (the longest tumor dimension) and W is tumor width (perpendicular to L). Body weights were also measured twice per week.

All mice were terminated at Day 26.

To compare tumor volumes of different groups at a pre-specified day, Bartlett's test was used to check the assumption of homogeneity of variance across all groups. When the p-value of Bartlett's test was 0.05, a one-way ANOVA was run to test overall equality of means across all groups. If the p-value of the one-way ANOVA was <0.05, a post hoc testing was performed by running Tukey's HSD (honest significant difference) tests for all pairwise comparisons, and Dunnett's tests for comparing each treatment group with the vehicle group. When the p-value of Bartlett's test was <0.05, a Kruskal-Wallis test was run to test overall equality of medians among all groups. If the p-value of the Kruskal-Wallis test was <0.05, a post hoc testing was performed by running Conover's non-parametric test for all pairwise comparisons or for comparing each treatment group with the vehicle group, both with single-step p-value adjustment.

In addition, pairwise comparisons were performed without multiple testing correction and nominal/uncorrected p-values were reported directly from Welch's t-test or Mann-Whitney U test. Specifically, Bartlett's test was first used to check the assumption of homogeneity of variance for a pair of groups. When the p-value of Bartlett's test was ≥0.05, Welch's t-test was performed; otherwise, a Mann-Whitney U test was performed, to obtain nominal p-values.

### 2 Results

After four doses of Doxycycline treatment, the single intravenous dose of AB2-RL4-LP1 at 4 mg/kg (Group 7) and the single intravenous dose of AB2-RL4-LP3 at 1 mg/kg (Group 8) demonstrated statistically significant efficacy against model RM1-hPSMAg (p<0.05) on Day 26. Without any Doxycycline treatment, the single intravenous dose of AB2-RL4-LP3 at 1 mg/kg also demonstrated statistically significant efficacy against model RM1-hPSMAg (p<0.05) on Day 26. Results are shown in Table 29 below.

The mice tolerated the treatments well. Mice dosed with SEB-LP3 ADC, AB2-RL4-LP1, or AB2-RL4-LP3 showed body weight loss that was recovered after the mice were provided with diet gels and subcutaneous fluid supplement for three consecutive days. For the other mice treatment groups, no body weight loss was observed.

TABLE 29
Antitumor Activity of Test ADCs in the Treatment
of model RM1-hPSMAg in C57BL/6 Mice

| | On Day 5 | On Day 26 | | | |
|---|---|---|---|---|---|
| | Mean | | Vehicle Group 1 as Control Group | | |
| | Tumor | Mean Tumor | T/C | | |
| Group | Size (mm³) | Size (mm³) | (%) | TGI (%) | p value |
| 1 | 84.59 ± 1.99 | 578.84 ± 204.09 | — | — | — |
| 2 | 84.56 ± 2.01 | 256.44 ± 64.29 | 44.30 | 65.23 | 0.874 |
| 3 | 84.57 ± 2.00 | 132.41 ± 39.51 | 22.88 | 90.32 | 0.073 |
| 4 | 84.55 ± 1.97 | 142.47 ± 47.42 | 24.61 | 88.28 | 0.107 |
| 5 | 84.58 ± 1.98 | 599.41 ± 144.67 | 103.55 | −4.16 | 1.000 |

| | On Day 5 | | | | |
|---|---|---|---|---|---|
| | Mean | On Day 26 | | | |
| | Tumor | | Vehicle Group 6 as Control Group | | |
| | Size | Mean Tumor | T/C | | |
| Group | (mm³)^{a} | Size (mm³)^{a} | (%) | TGI (%) | p value |
| 6 | 84.57 ± 1.99 | 863.76 ± 197.56 | — | — | — |
| 7 | 84.55 ± 1.97 | 167.26 ± 37.15 | 19.36 | 89.39 | 0.005** |
| 8 | 84.56 ± 1.99 | 192.88 ± 31.52 | 22.33 | 86.10 | 0.039* |

| | On Day 5 | | | | |
|---|---|---|---|---|---|
| | Mean | On Day 26 | | | |
| | Tumor | | Vehicle Group 9 as Control Group | | |
| | Size | Mean Tumor | T/C | | |
| Group | (mm³)^{a} | Size (mm³)^{a} | (%) | TGI (%) | P value |
| 9 | 84.59 ± 1.99 | 1592.85 ± 191.34 | — | — | — |
| 10 | 84.60 ± 2.11 | 282.18 ± 72.68 | 17.72 | 86.90 | <0.001*** |

### Example 12. In Vivo Anti-Tumor Activities of Anti-PSMA ADCs and Cytokine Production after ADC Treatment In Vivo

Anti-PSMA antibody deJ591 was conjugated with the STING agonist Compound 1 or Compound 2. Two different attachment points on Compound 1 or Compound 2 were used for linker attachment to the sulfur on the (S)-thiophosphate (“S-linked”) or to the bridge nitrogen (“N-linked”). Various structural changes were made to the linkers to assess the effects of spacer, cleavage site, and second-spacer (second immolative payload release step) modifications on potency, toxicity, and stability of the resultant ADCs. This example shows in vivo anti-tumor activity of anti-PSMA ADCs in tumor-bearing mice and assesses pharmacodynamic (PD) effects by measuring mouse cytokine production, including type I interferons.

### 1 Summary of methods

### 1.1 List of Anti-PSMA-LP ADCs

Anti-PSMA conjugated to linker-payload (LP) evaluated in this example are listed below in Table 30.

TABLE 30
List of Anti-PSMA-LP ADCs

| LP | Antibody | DAR |
|---|---|---|
| LP2 | a-hPSMA-zuJ591-H14L5-hIgG1-A118C C80 | 4 |
| LP3 | a-hPSMA-zuJ591-H14L5-hIgG1-A118C C80 | 3.81 |
| LP4 | a-hPSMA-zuJ591-H14L5-hIgG1-A118C C80 | 3.9 |
| LP5 | a-hPSMA-zuJ591-H14L5-hIgG1-A118C C80 | 3.9 |
| LP6 | a-hPSMA-zuJ591-H14L5-hIgG1-A118C C80 | 3.92 |
| LP7 | a-hPSMA-zuJ591-H14L5-hIgG1-A118C C80 | 4.02 |
| LP8 | a-hPSMA-zuJ591-H14L5-hIgG1-A118C C80 | 4.06 |
| LP9 | a-hPSMA-zuJ591-H14L5-hIgG1-A118C C80 | 3.96 |
| LP10 | a-hPSMA-zuJ591-H14L5-hIgG1-A118C C80 | 3.85 |
| LP11 | a-hPSMA-zuJ591-H14L5-hIgG1-A118C C80 | 2.5 |
| LP12 | a-hPSMA-zuJ591-H14L5-hIgG1-A118C C80 | 3.96 |
| LP13 | a-hPSMA-zuJ591-H14L5-hIgG1-A118C C80 | 3.78 |
| LP14 | a-hPSMA-zuJ591-H14L5-hIgG1-A118C C80 | 3.81 |
| LP15 | a-hPSMA-zuJ591-H14L5-hIgG1-A118C C80 | 4.04 |
| LP16 | a-hPSMA-zuJ591-H14L5-hIgG1-A118C C80 | 4.02 |
| LP17 | a-hPSMA-zuJ591-H14L5-hIgG1-A118C C80 | 4 |
| LP18 | a-hPSMA-zuJ591-H14L5-hIgG1-A118C C80 | 4 |
| LP19 | a-hPSMA-zuJ591-H14L5-hIgG1-A118C C80 | 4 |
| LP20 | a-hPSMA-zuJ591-H14L5-hIgG1-A118C C80 | 3.78 |
| LP21 | a-hPSMA-zuJ591-H14L5-hIgG1-A118C C80 | 4 |
| LP22 | a-hPSMA-zuJ591-H14L5-hIgG1-A118C C80 | 3.14 |
| LP23 | a-hPSMA-zuJ591-H14L5-hIgG1-A118C C80 | 3.2 |
| LP24 | a-hPSMA-zuJ591-H14L5-hIgG1-A118C C80 | 4 |
| LP25 | a-hPSMA-zuJ591-H14L5-hIgG1-A118C C80 | 3.84 |
| LP26 | a-hPSMA-zuJ591-H14L5-hIgG1-A118C C80 | 3.88 |
| LP27 | a-hPSMA-zuJ591-H14L5-hIgG1-A118C C80 | 4 |
| LP28 | a-hPSMA-zuJ591-H14L5-hIgG1-A118C C80 | 3.93 |
| LP29 | a-hPSMA-zuJ591-H14L5-hIgG1-A118C C80 | 4 |
| LP30 | a-hPSMA-zuJ591-H14L5-hIgG1-A118C C80 | 3.96 |
| LP31 | a-hPSMA-zuJ591-H14L5-hIgG1-A118C C80 | 3.85 |
| LP32 | a-hPSMA-zuJ591-H14L5-hIgG1-A118C C80 | 4 |

### 1.2 In Vivo Efficacy in the 22Rv1 Xenograft Model [Cohort 1]

Male BALB/c Nude mice aged 6 weeks (Charles River) were used for this study. 22Rv1 cells (Lot: RJT07NOV22p14) were grown in RPMI medium containing 10% HI-FBS. 5×10⁶ 22Rv1 cells in 1×PBS containing 50% Matrigel were injected subcutaneously in the right flank of the mice at a volume of 200 μL. Tumor growth was monitored via caliper to reach an average of 150-200 mm³ (~161 mm³), then tumor-bearing mice were randomized into treatment and control groups of 6 mice per group (12 groups for Cohort 1, 11 groups for Cohort 2, and 15 groups for Cohort 3, as detailed below). The enrolled mice received a single intravenous (IV) injection of 100 μL vehicle or drug. Blood samples were collected via submandibular (facial) vein into serum collection tubes. Blood was allowed to clot at room temperature, then serum was isolated by centrifugation at 1,200 g for 15 min at 4° C. Isolated serum was transferred to clean polypropylene tubes and stored at −80° C. Tumor volume and body weight were monitored for 26 days (Cohort 1), 35 days (Cohort 2), or 21 days (Cohort 3).

TABLE 31
22Rv1 Dosing groups - Cohort 1

| | | Dose | | |
|---|---|---|---|---|
| Group | Compound | (mg/kg) | Route | Regimen |
| 1 | Vehicle (PBS) | 0 | IV | Single |
| 2 | Anti-PSMA-LP2 | 1.0 | IV | Single |
| 3 | Anti-PSMA-LP3 | 2.0 | IV | Single |
| 4 | Anti-PSMA-LP8 | 2.0 | IV | Single |
| 5 | Anti-PSMA-LP10 | 2.0 | IV | Single |
| 6 | Anti-PSMA-LP11 | 3.2 | IV | Single |
| 7 | Anti-PSMA-LP13 | 2.0 | IV | Single |
| 8 | Anti-PSMA-LP14 | 2.0 | IV | Single |
| 9 | Anti-PSMA-LP15 | 2.0 | IV | Single |
| 10 | Anti-PSMA-LP16 | 2.0 | IV | Single |
| 11 | Anti-PSMA-LP20 | 2.0 | IV | Single |
| 12 | Anti-PSMA-LP28 | 2.0 | IV | Single |

TABLE 32
22Rv1 Dosing groups - Cohort 2

| | | Dose | | |
|---|---|---|---|---|
| Group | Compound | (mg/kg) | Route | Regimen |
| 1 | Vehicle (PBS) | 0 | IV | Single |
| 2 | Anti-PSMA-LP2 | 1.0 | IV | Single |
| 3 | Anti-PSMA-LP3 | 2.0 | IV | Single |
| 4 | Anti-PSMA-LP4 | 2.0 | IV | Single |
| 5 | Anti-PSMA-LP5 | 2.0 | IV | Single |
| 6 | Anti-PSMA-LP6 | 2.0 | IV | Single |
| 7 | Anti-PSMA-LP7 | 2.0 | IV | Single |
| 8 | Anti-PSMA-LP9 | 2.0 | IV | Single |
| 9 | Anti-PSMA-LP12 | 2.0 | IV | Single |
| 10 | Anti-PSMA-LP16 | 2.0 | IV | Single |
| 11 | Anti-PSMA-LP30 | 2.0 | IV | Single |

TABLE 33
22Rv1 Dosing groups - Cohort 3

| | | Dose | | |
|---|---|---|---|---|
| Group | Compound | (mg/kg) | Route | Regimen |
| 1 | Vehicle (PBS) | 0.00 | IV | Single |
| 2 | Anti-PSMA-LP2 | 2.00 | IV | Single |
| 3 | Anti-PSMA-LP17 | 2.00 | IV | Single |
| 4 | Anti-PSMA-LP18 | 2.00 | IV | Single |
| 5 | Anti-PSMA-LP19 | 2.00 | IV | Single |
| 6 | Anti-PSMA-LP21 | 2.00 | IV | Single |
| 7 | Anti-PSMA-LP22 | 2.50 | IV | Single |
| 8 | Anti-PSMA-LP23 | 2.54 | IV | Single |
| 9 | Anti-PSMA-LP24 | 2.00 | IV | Single |
| 10 | Anti-PSMA-LP25 | 2.00 | IV | Single |
| 11 | Anti-PSMA-LP26 | 2.00 | IV | Single |
| 12 | Anti-PSMA-LP27 | 2.00 | IV | Single |
| 13 | Anti-PSMA-LP29 | 2.00 | IV | Single |
| 14 | Anti-PSMA-LP31 | 2.00 | IV | Single |
| 15 | Anti-PSMA-LP32 | 2.00 | IV | Single |

### 1.3 In Vivo Efficacy in C4-2 Xenograft Model

Male Fox Chase SCID mice aged 6 weeks (Envigo) were used for this study. C4-2 cells (Lot: RJT07NOV22p11) were grown in RPMI medium containing 10% HI-FBS. 1×10⁷′ C4-2 cells in 1×PBS containing 50% Matrigel were injected subcutaneously in the right flank of the mice at a volume of 200 μL. Tumor growth was monitored via caliper to reach an average of 150-200 mm³ (~170 mm³), then tumor bearing mice were randomized into treatment and control groups of 5 mice per group (6 groups, detailed below). The enrolled mice received a single intravenous (IV) injection of 100 μL vehicle or drug. Blood samples were collected via submandibular (facial) vein into serum collection tubes. Blood was allowed to clot at RT, then serum was isolated by centrifugation at 2000×g for 15 min at 4° C. Isolated serum was transferred to clean polypropylene tubes and stored at −80° C. Tumor volume and body weights were monitored for 35 days.

TABLE 34
C4-2 Dosing groups

| | | Dose | | |
|---|---|---|---|---|
| Group | Compound | (mg/kg) | Route | Regimen |
| 1 | Vehicle (PBS) | 0 | IV | Single |
| 2 | Anti-PSMA-LP2 | 1.0 | IV | Single |
| 3 | Anti-PSMA-LP14 | 2.0 | IV | Single |
| 4 | Anti-PSMA-LP26 | 2.0 | IV | Single |
| 5 | Anti-PSMA-LP29 | 2.0 | IV | Single |
| 6 | Anti-PSMA-LP28 | 2.0 | IV | Single |

### 1.4 Serum and Plasma Cytokine Analysis

Serum and plasma samples were collected six hours post-treatment and stored at−80° C. until analysis. Samples were assayed using Luminex xMAP multiplex kits (Invitrogen #PPX-08-MX2XANK) designed to detect and quantify the following cytokines: IFN-β, IFN-γ, IL-6, IL-13, IP-10 (CXCL10), MCP-1 (CCL2), MIP-1a (CCL3), and MIP-1p (CCL4). All reagents were prepared according to the manufacturer's guidelines. Standards and samples were incubated overnight with magnetic beads and all washing steps were performed using an Agilent automatic plate washer equipped with a magnet insert. Data was collected using a Luminex xMAP Intelliflex and analyzed using EMD Millipore's Belysa software.

### 1.5 Statistical Analyses

Statistical analysis of differences in average tumor volume between vehicle-treated and anti-PSMA-LP ADC treated groups was performed using two-way ANOVA to test overall equality of means across all groups. Groups were examined for all pairwise comparisons using Dunnett method.

### 2 Results

### 2.1 Anti-Tumor Activity of Anti-PSMA LP ADCs in 22Rv1 Xenograft Mouse Model and Serum Cytokine Analysis (Cohort 1)

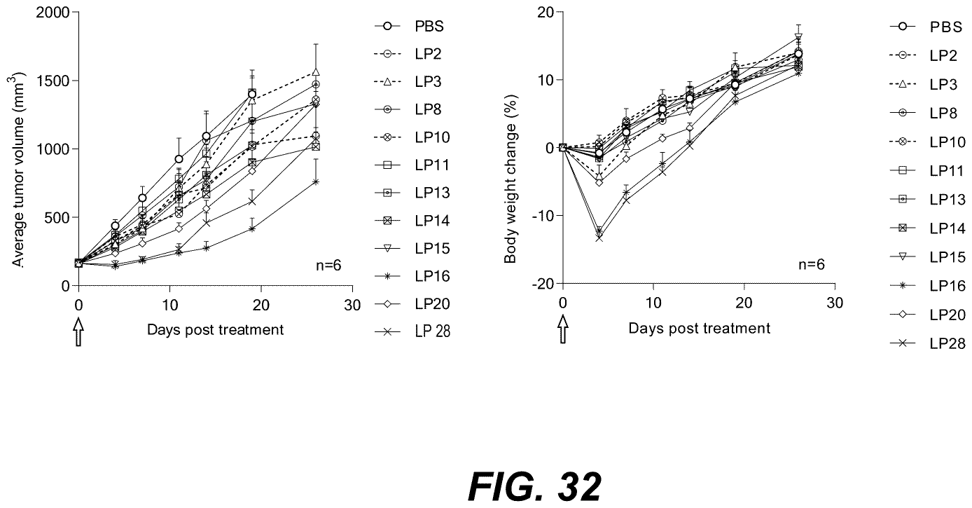

Anti-PSMA-LP ADCs (random DAR2~4) were administered as a single IV bolus dose to 22Rv1 xenograft in male BALB/c nude mice model. Averaged tumor volumes of treatment groups were compared to control group 1 (vehicle/PBS) and body weight changes (normalized to Day 0) were also measured. The results of this analysis are shown in FIG. 32. The analysis demonstrated significant anti-tumor activity of anti-PSMA-LP16, -LP28, -LP20 and -LP10 on Day 11 post dose. Continuous anti-tumor activities of anti-PSMA-LP16 (mcVAPC-Unit 8-SN-Compound 1) and -LP28 (mcVAPC-Unit 11-SN-Compound 1) were observed on Day 19 post dose. Anti-PSMA-LP16 (mcVAPC-Unit 8-SN-Compound 1) and -LP28 (mcVAPC-Unit 11-SN-Compound 1) treated mice showed temporary body weight loss of over 10% by Day 4, while anti-PSMA-LP3 (mcVAP-Sp-Compound 1) and -LP20 (mcVAPC-Unit 9-SN-Compound 1) showed temporary body weight loss of 5% by Day 4. Other anti-PSMA-LP ADCs tested showed limited body weight loss.

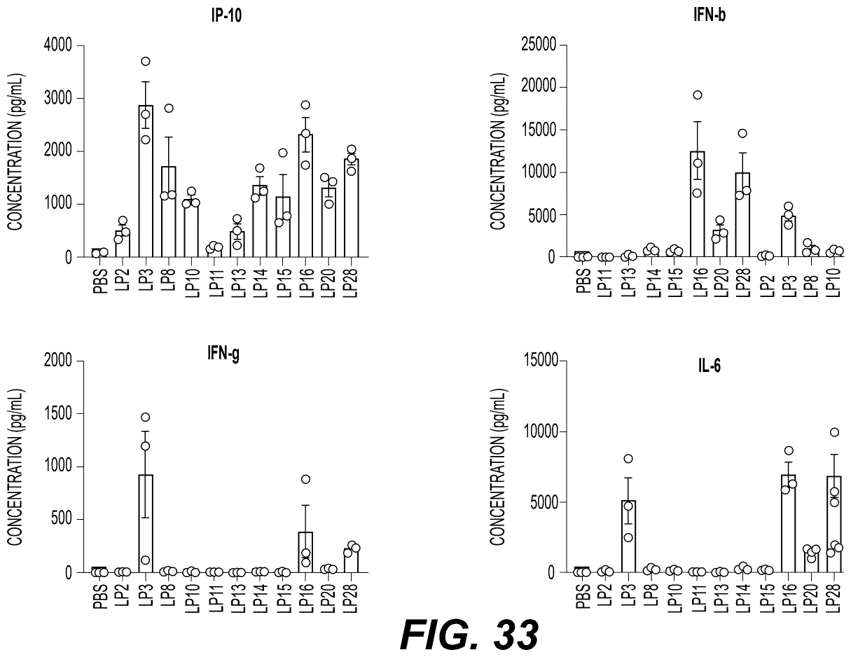

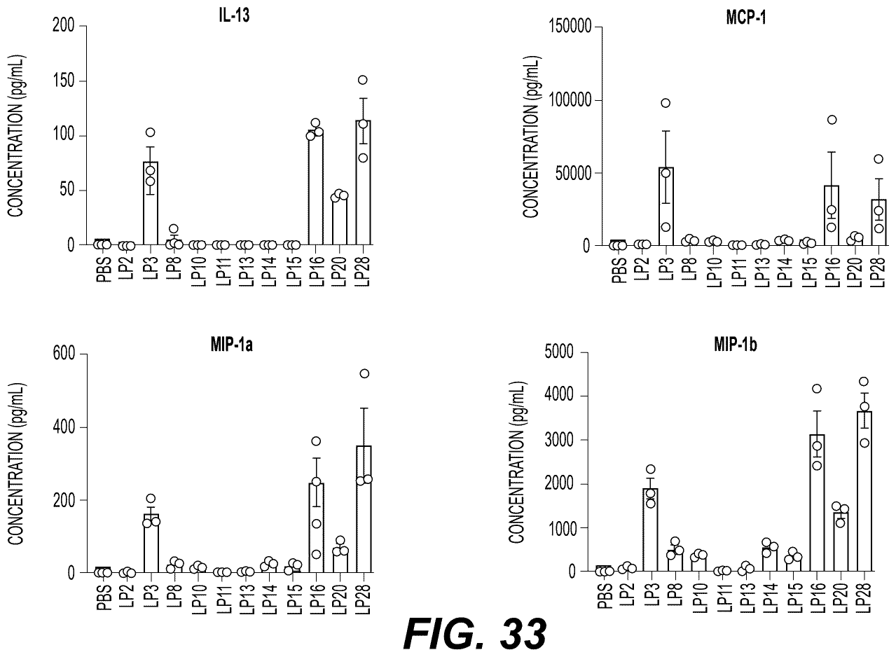

For serum cytokine analysis, mouse serum was collected 6 hours post treatment. Cytokine concentrations were measured and quantified using a Luminex xMAP multiplex kit. The results of this analysis are shown in FIG. 33 and Table 35. The analysis demonstrated production of cytokines six hours post dose. These data show that anti-PSMA-LP16 and -LP28, which had desirable anti-tumor activity, also stimulated abundant production of cytokines. Treatment with either anti-PSMA-LP16 or -LP28 resulted in temporary body weight loss over 10%. Anti-PSMA-LP3 (mcVAP-Sp-Compound 1) also stimulated abundant production of cytokines. While anti-PSMA-LP3 group had low anti-tumor activity, it also resulted in acute body weight loss. The anti-PSMA-[P10 and -LP20 ADCs showed anti-tumor activity, and also stimulated cytokine production.

TABLE 35
Summary of Anti-tumor activities and Cytokine production in anti-PSMA-LP ADCs in the
22Rv1 xenograft model (Cohort 1)

| | | Tier* | | | | | | | | | | |
|---|---|---|---|---|---|---|---|---|---|---|---|---|
| | | Tier 1 | | | | Tier 2 | Tier 3 | | | Out** | | |
| | | LP# | | | | | | | | | | |
| | | 16 | 28 | 20 | 10 | 14 | 2 | 13 | 15 | 3 | 8 | 11 |
| | | BW Loss (%) | | | | | | | | | | |
| | | 10 | 10 | 5 | | | | | | 5 | | |
| Cytokines | IP10 | +++ | ++ | ++ | ++ | ++ | + | + | ++ | +++ | ++ | +/− |
| | IFN-β | +++ | +++ | ++ | + | + | +/− | +/− | + | ++ | + | − |
| | IFN-γ | ++ | + | +/− | +/− | +/− | − | +/− | +/− | +++ | +/− | +/− |
| | IL-6 | ++ | +++ | + | +/− | +/− | − | − | +/− | +++ | +/− | − |
| | IL-13 | +++ | +++ | ++ | − | − | +/− | − | − | +++ | +/− | − |
| | MCP-1 | +++ | +++ | + | +/− | +/− | +/− | − | +/− | +++ | +/− | − |
| | MIP-1α | +++ | +++ | + | +/− | +/− | − | − | +/− | +++ | +/− | − |
| | MIP-1β | +++ | +++ | ++ | + | + | +/− | +/− | + | +++ | + | +/− |
| Anti- | p < 0.0001 on D11 | TRUE | TRUE | TRUE | TRUE | | | | | | | |
| Tumor | p < 0.001 on D11 | TRUE | TRUE | TRUE | TRUE | TRUE | | | | | | |
| Activities | p < 0.05 on D11 | TRUE | TRUE | TRUE | TRUE | TRUE | TRUE | TRUE | TRUE | | | |
| | No significance | | | | | | | | | TRUE | TRUE | TRUE |

*Tier: Classified LPs based on anti-tumor activity
**Out: Out of Tier without meaningful anti-tumor activity on Day 11 (D11)

### 2.2 Anti-Tumor Activity of Anti-PSMA LP ADCs in 22Rv1 Xenograft Mouse Model and Serum Cytokine Analysis (Cohort 2)

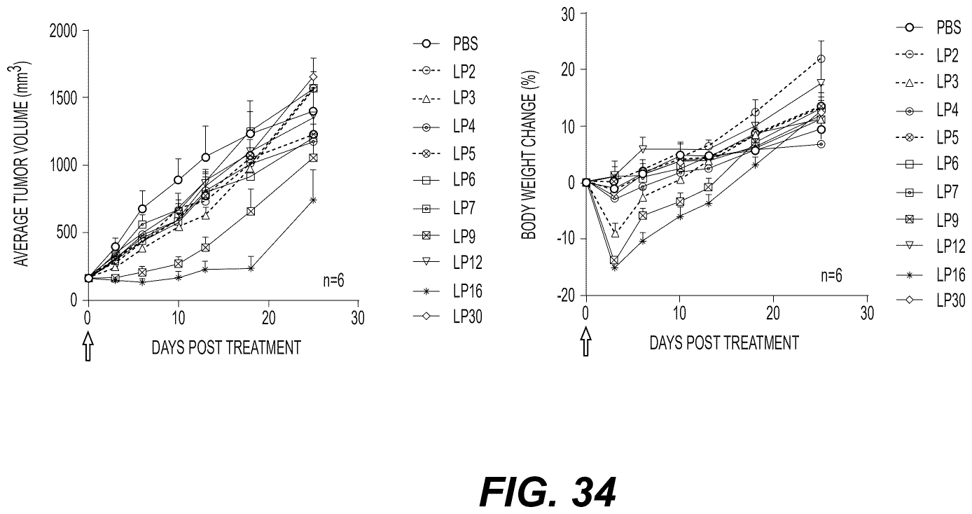

Anti-PSMA-LP ADCs (random DAR2~4) were administered as a single IV bolus dose to 22Rv1 xenograft in male BALB/c nude mice model. Averaged tumor volumes of treatment groups were compared with control group 1 (vehicle/PBS) and body weight changes (normalized to Day 0) were also measured. The results of this analysis are shown if FIG. 34. The analysis demonstrated statistically significant anti-tumor activity in groups treated with anti-PSMA-LP3, -LP9, and -LP16 on Day 10 and weak statistical significance in groups treated with anti-PSMA-LP2, -LP4, -LP5, -LP6, -LP7 and -LP12 (p<0.05), while the group treated with anti-PSMA-LP30 showed less anti-tumor activity. Sustained anti-tumor activity seen in groups treated with anti-PSMA-LP3 (mcVAP-Sp-Compound 1) and anti-PSMA-LP9 (mcVAPC-SN-Compound 1) were observed until Day 17 post dose. Anti-PSMA-LP3, -LP9, and -LP16-treated mice showed temporary body weight loss over 10% by Day 4.

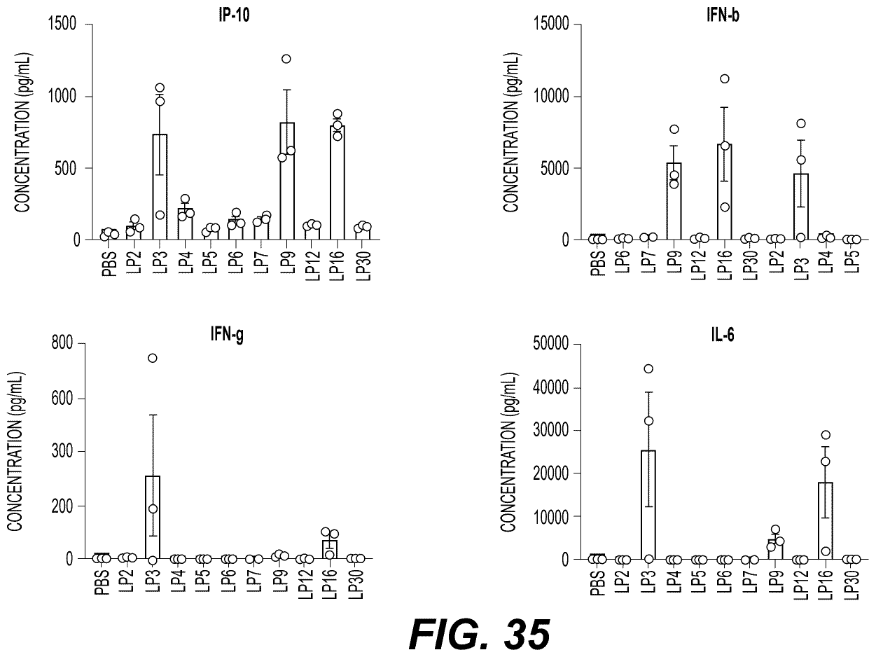

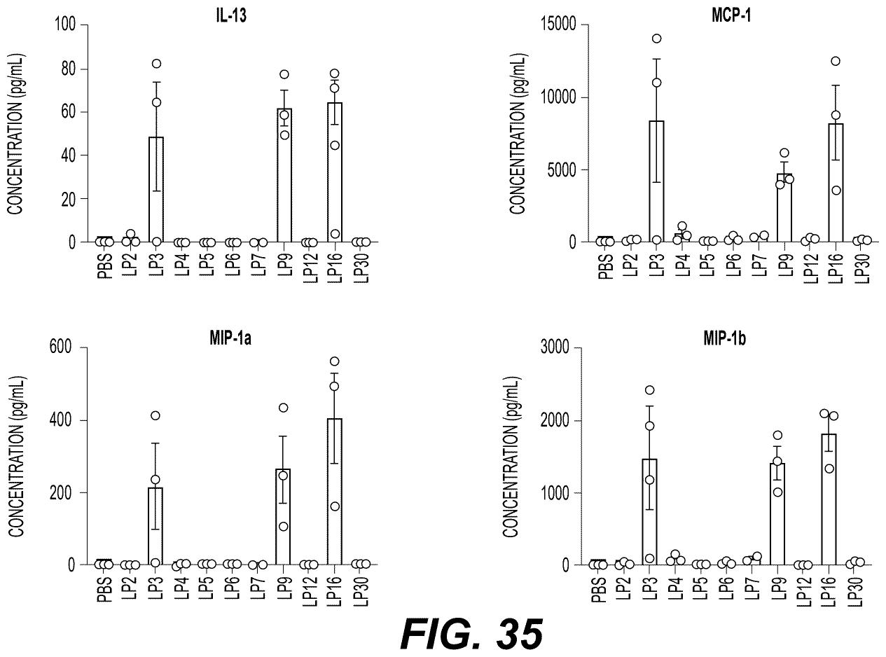

Mouse serum was collected 6 hours post treatment. Cytokine concentrations were measured and quantified using a Luminex xMAP multiplex kit. The results of this analysis are shown in FIG. 35 and Table 36. The analysis demonstrated production of cytokines six hours post dose. It is noted that anti-PSMA-LP9 (mcVAPC-SN-Compound 1) and LP16 (as same as cohort1), having superior anti-tumor activity, produced cytokines with a similar abundancy to anti-PSMA-LP3 but showed acute body weight loss. On the other hand, Tier3 groups (anti-PSMA-LP2, -LP4, -LP5, -LP6, -LP7 and -LP12) also produced cytokines but lower level compared to Tiers 1 and 2.

TABLE 36
Summary of Anti-tumor activities and Cytokine production in anti-PSMA-LP ADCs in the
22Rv1 xenograft model (Cohort 2)

| | | Tier* | | | | | | | | | |
|---|---|---|---|---|---|---|---|---|---|---|---|
| | | Tier 1 | | Tier 2 | Tier 3 | | | | | | Out** |
| | | LP# | | | | | | | | | |
| | | 16 | 9 | 3 | 2 | 4 | 5 | 6 | 7 | 12 | 30 |
| | | BW Loss (%) | | | | | | | | | |
| | | 10 | 10 | 10 | | | | | | | |
| Cytokines | IP10 | +++ | +++ | +++ | + | ++ | + | ++ | ++ | + | + |
| | IFN-β | +++ | +++ | +++ | +/− | + | − | +/− | +/− | +/− | +/− |
| | IFN-γ | ++ | + | +++ | − | − | − | − | − | +/− | − |
| | IL-6 | +++ | + | +++ | − | − | − | − | − | − | − |
| | IL-13 | +++ | +++ | +++ | +/− | − | − | − | − | − | − |
| | MCP-1 | +++ | +++ | +++ | +/− | +/− | +/− | +/− | +/− | +/− | +/− |
| | MIP-1α | +++ | +++ | +++ | − | +/− | − | − | − | − | − |
| | MIP-1β | +++ | +++ | +++ | +/− | +/− | +/− | +/− | +/− | +/− | +/− |
| Anti- | p < 0.0001 on D10 | TRUE | TRUE | | | | | | | | |
| Tumor | p < 0.001 on D10 | TRUE | TRUE | TRUE | | | | | | | |
| Activities | p < 0.05 on D10 | TRUE | TRUE | TRUE | TRUE | TRUE | TRUE | TRUE | TRUE | TRUE | |
| | No significance | | | | | | | | | | TRUE |

*Tier: Classified LPs based on anti-tumor activity
**Out: Out of Tier without meaningful anti-tumor activity on Day 10 (D10)

### 2.3 Anti-Tumor Activity of Anti-PSMA LP ADCs in 22Rv1 Xenograft Mouse Model and Serum Cytokine Analysis (Cohort 3)

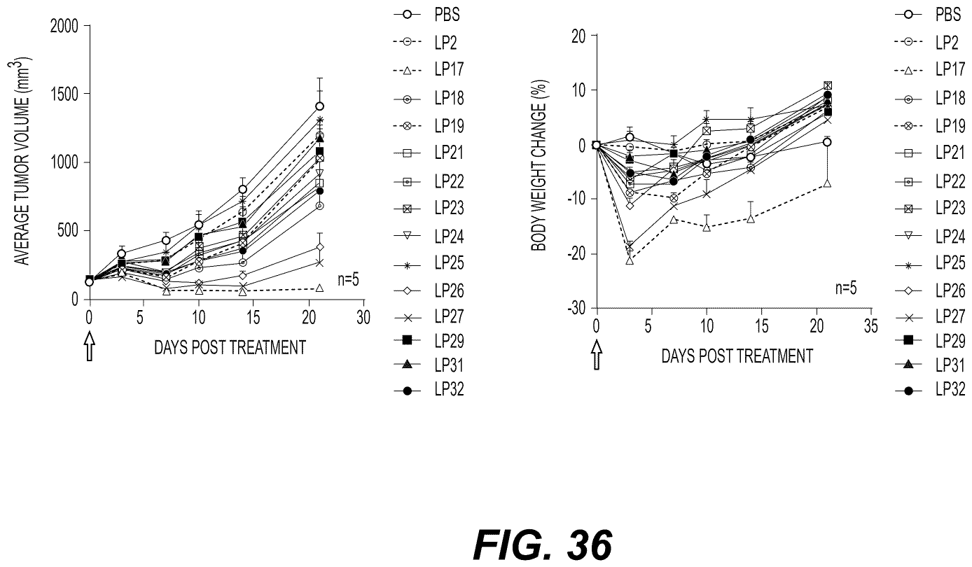

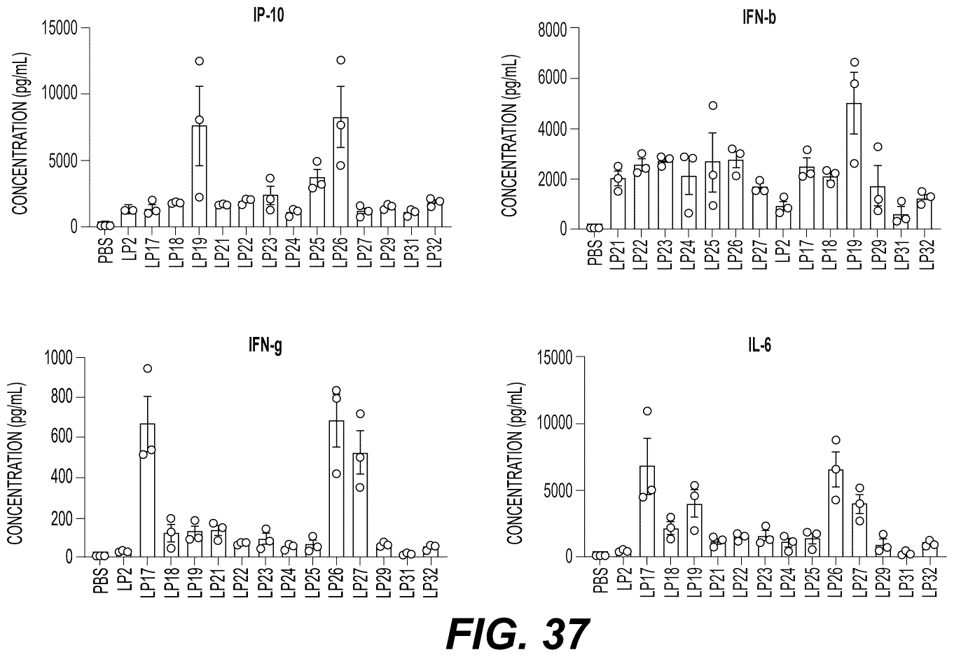

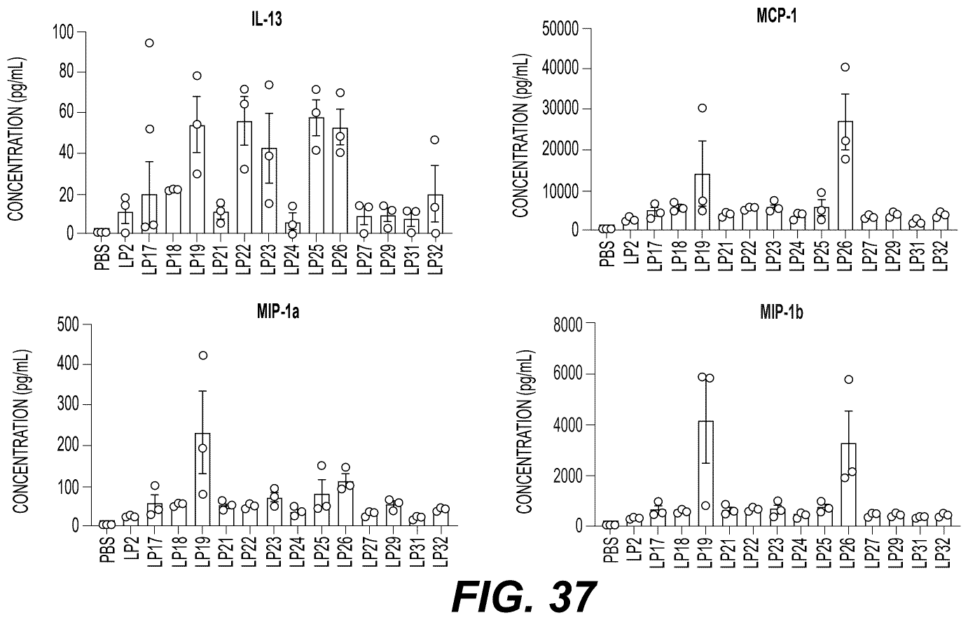

Anti-PSMA-LP ADCs (random DAR2~4) were administered as a single IV bolus dose to 22Rv1 xenograft tumor-bearing male BALB/c nude mice. Averaged tumor volumes of treatment groups were compared with control group 1 (vehicle/PBS) and body weight changes (normalized to Day 0) were also measured. The results of this analysis are shown in FIG. 36. The analysis demonstrated statistically superior anti-tumor activity of the anti-PSMA-LP17 and -LP27 (p<0.0001) beginning on Day 7 and significant anti-tumor activities for groups treated with anti-PSMA-LP17, -LP18, -LP26, -LP27, -LP32 (p<0.0001), and -LP21 and -LP22 (P<0.001) on Day 21 post-dose. There was also significant anti-tumor activity (p<0.05) in mice treated with anti-PSMA-LP24 (mcGGFG-Unit 9-SN-Compound 1) observed by Day 21 post dose. Overall, three anti-PSMA-ADCs (LP-17, -27 and -26) showed strong anti-tumor activities in the study. Of note, anti-PSMA-LP17 and -LP27 showed temporary body weight loss of 20% or over by Day 3 and around 10% body weight loss in mice treated with anti-PSMA-LP26, -LP19, -LP18 ADCs.

Mouse plasma was collected 6 hours post treatment. Cytokine concentrations were measured and quantified using a Luminex xMAP multiplex kit. The results of this analysis are shown in FIG. 37 and Table 37. Cytokine production was observed across all anti-PSMA-LP ADCs. The anti-PSMA-LP19-treated mice produced higher amounts of most cytokines examined, while there was limited anti-tumor activity and relatively high body weight loss. On the other hand, three anti-PSMA-ADCs (LP-17, -27 and -26) with strong anti-tumor activities also produced higher levels of cytokines such as IFN-γ, IL-6, and IL-13, but anti-PSMA-LP26 stimulated production of more cytokines than the other two anti-PSMA-LP17 and -LP27 ADCs in this study. Of note, anti-PSMA-LP26 showed temporary body weight loss of 10%, while over 20% body weight loss was seen in mice treated with anti-PSMA-LP17 and -27 ADCs.

TABLE 37
Summary of Anti-tumor activities and Cytokine production in anti-PSMA-LP ADCs in the
22Rv1 xenograft model (Cohort 3)

| | | Tier* | | | | | | | | |
|---|---|---|---|---|---|---|---|---|---|---|
| | | Tier 1 | | | Tier 2 | | Tier 3 | | | Out** |
| | | LP# | | | | | | | | |
| | | 17 | 27 | 26 | 18 | 32 | 21 | 22 | 24 | 19 |
| | | BW Loss (%) | | | | | | | | |
| | | 20 | 20 | 10 | 10 | | | | | 10 |
| Cytokines | IP10 | + | + | +++ | + | + | + | + | + | +++ |
| | IFN-β | ++ | ++ | ++ | ++ | ++ | ++ | ++ | ++ | +++ |
| | IFN-γ | +++ | +++ | +++ | + | + | + | + | + | + |
| | IL-6 | +++ | ++ | +++ | + | + | + | + | + | ++ |
| | IL-13 | ++ | + | +++ | ++ | ++ | + | +++ | + | +++ |
| | MCP-1 | + | + | +++ | + | + | + | + | + | ++ |
| | MIP-1α | + | + | ++ | + | + | + | + | + | +++ |
| | MIP-1β | + | + | +++ | + | + | + | + | + | +++ |
| Anti- | p < 0.0001 on D7 | TRUE | TRUE | | | | | | | |
| Tumor | P < 0.0001 on D21 | TRUE | TRUE | TRUE | TRUE | TRUE | | | | |
| Activities | p <0.001 on D21 | TRUE | TRUE | TRUE | TRUE | TRUE | TRUE | TRUE | | |
| | p <0.05 on D21 | TRUE | TRUE | TRUE | TRUE | TRUE | TRUE | TRUE | TRUE | |
| | No significance | | | | | | | | | TRUE |

*Tier: Classified LPs based on the day 21 (D21) anti-tumor activity
**Out: Out of Tier without meaningful anti-tumor activity on D21

### 2.4 Anti-Tumor Activity of Anti-PSMA-LP ADCs in the C4-2 Xenograft SCID Mouse Model

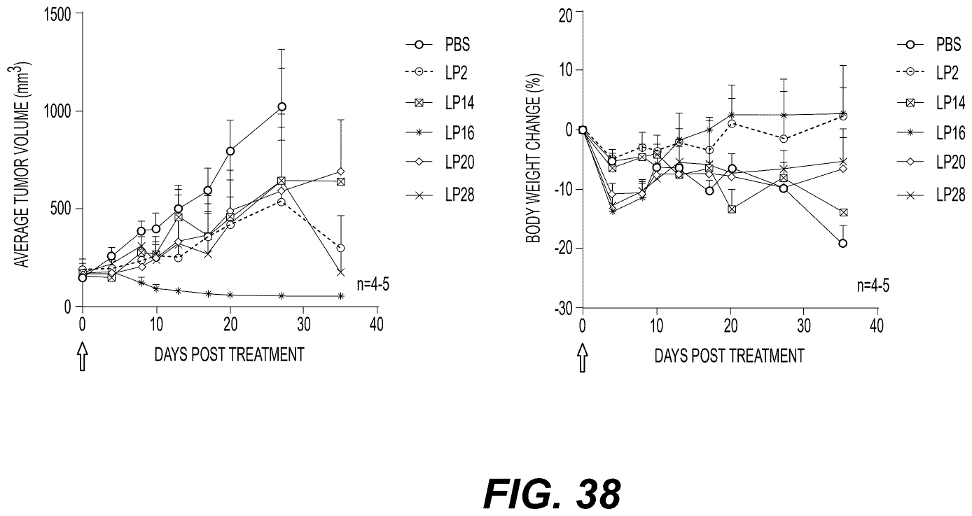

Anti-PSMA-LP ADCs (random DAR2~4) were administered as a single IV bolus dose to C4-2 xenograft tumor-bearing male SCID mice. Averaged tumor volumes of treatment groups were compared with control group 1 (vehicle/PBS) and body weight changes (normalized to Day 0) were also measured. The results of this analysis are shown in FIG. 38. The analysis demonstrated statistically superior anti-tumor activity of the anti-PSMA-LP16 (mcVAPC-Unit 8-SN-Compound 1) beginning on Day 10 post-dose and its anti-tumor activities continued to the end point. There was also significant anti-tumor activity (P<0.05) in mice dosed with anti-PSMA-LP28 (mcVAPC-Unit 11-SN-Compound 1) by Day 17 post-dose as gaussian distributions range. In this model, anti-tumor activity was seen in anti-PSMA-LP2, -LP14 groups though not significant on Day 17. While anti-PSMA-LP16, -LP20 and -LP28 treated mice showed temporary body weight loss of over 10% by Day 4, other anti-PSMA-LP ADCs tested showed limited body weight loss in the C4-2 model.

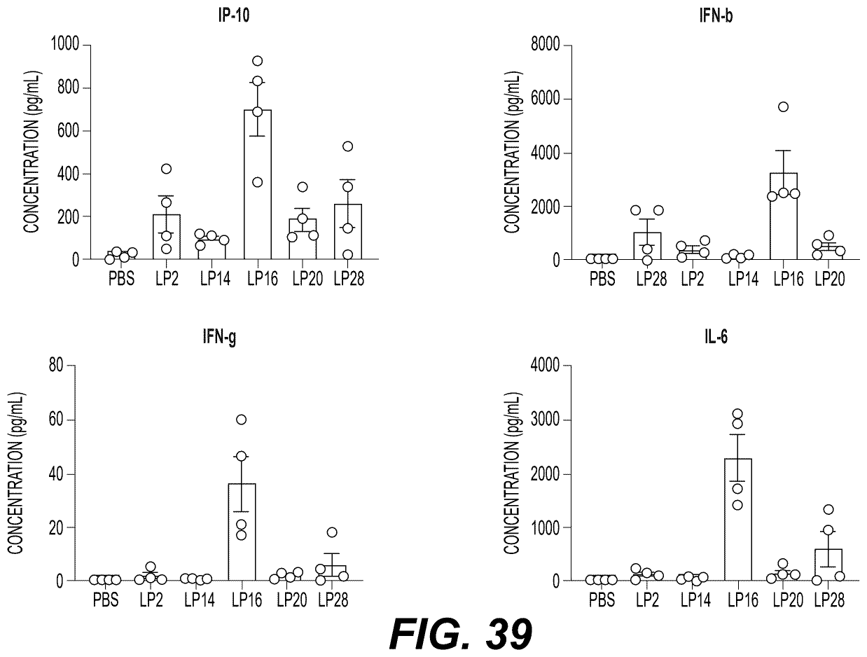

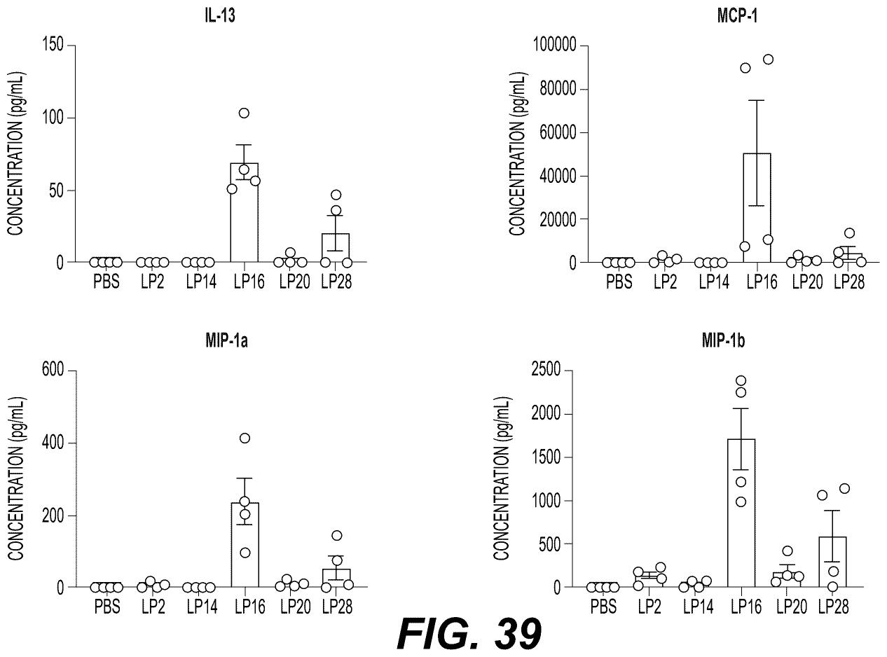

Mouse serum was collected 6 hours post treatment. Cytokine concentrations were measured and quantified using a Luminex xMAP multiplex kit. The results of this analysis are shown in FIG. 39 and Table 38. Cytokine production was observed across all anti-PSMA-LP ADCs, and cytokine production in anti-PSMA-LP16, LP28, -LP20, -LP2 and -LP14 was ranked in this C4-2 xenograft mouse model. anti-PSMA-LP16 and -LP28, which showed statistically significant anti-tumor activities, also stimulated abundant cytokine production in this model.

TABLE 38
Summary of Anti-tumor activities and Cytokine production in
anti-PSMA-LP ADCs in the C4-2 xenograft model

| | | Tier* | | | | |
|---|---|---|---|---|---|---|
| | | Tier 1 | Tier 2 | Tier 3 | | |
| | | LP# | | | | |
| | | 16 | 28 | 2 | 14 | 20 |
| | | BW Loss (%) | | | | |
| | | 10 | 10 | | | 10 |
| Cytokines | IP10 | +++ | ++ | ++ | + | + |
| | IFN-β | +++ | ++ | + | + | + |
| | IFN-γ | +++ | + | + | + | + |
| | IL-6 | +++ | + | + | + | + |
| | IL-13 | +++ | ++ | − | − | + |
| | MCP-1 | +++ | +/− | +/− | − | +/− |
| | MIP-1α | +++ | ++ | +/− | − | +/− |
| | MIP-1β | +++ | ++ | + | +/− | + |
| Anti- | p < 0.0001 | TRUE | TRUE | | | |
| Tumor | on D10 | | | | | |
| Activities | p < 0.05 | TRUE | TRUE | | | |
| | on D17 | | | | | |
| | No significance | | | TRUE | TRUE | TRUE |

*Tier: Classified LPs based on anti-tumor activity

### Example 13. Pharmacokinetics of Anti-Human PSMA-LP3 ADCs in Mouse

### 1 Methods

### 1.1 Animal Dosing and Sample Collection (Non-Tumor Bearing Mice)

6-week-old male balb/c nude mice (Jackson Labs) were used to study pharmacokinetics (PK) in non-tumor bearing mice. Anti-PSMA-LP3 was diluted in PBS to a concentration of 5 mg ADC per kg mouse weight prior to dosing (1 mpk final dosing). Mice were intravenously injected with 200 μL of the anti-PSMA-LP3. Anesthesia cocktail was prepared with ketamine (0.9 mL) and xylazine (0.5 mL) in 0.9% saline (8.6 mL). Mice were anesthetized intraperitoneally with 200 μL of ketamine/xylazine mixture at 10 minutes, 30 minutes, 2 hours, 6 hours, 24 hours, 72 hours, 168 hours, and 336 hours post dose (3 mice per time point). Blood draws were taken by cardiac puncture using a 27 G needle with a 3 mL syringe, followed by immediate euthanasia by cervical dislocation. Collected blood was immediately transferred to sodium citrate tubes and plasma was collected after centrifugation at 2000 g for 15 min. Plasma was stored at−80° C. until use.

### 1.2 Animal Dosing and Sample Collection (Tumor-Bearing Mice)

C4-2 tumors (castration-resistant human prostate cancer LnCAP subline) were established subcutaneously in 6-week-old male CB17 SCID mice (Envigo). Anti-PSMA-LP3 was diluted in PBS to a concentration of 15 mg and 45 mg ADC per kg mouse weight prior to dosing (3 mpk and 9 mpk final dosing). Treatment began when the average tumor size reached ~100 mm³. Mice were intravenously injected with 200 μL of the anti-PSMA-LP3. Anesthesia cocktail was prepared with ketamine (0.9 mL) and xylazine (0.5 mL) in 0.9% saline (8.6 mL). Mice were anesthetized intraperitoneally with 200 μL of ketamine/xylazine mixture at 5 minutes, 30 minutes, 2 hours, 6 hours, 24 hours, 72 hours, 168 hours, and 504 hours post dose (3 mice per time point). Blood draws were taken by cardiac puncture using a 27 G needle with a 3 mL syringe, followed by immediate euthanasia by cervical dislocation. Tumors and selected organs were isolated by dissection, flash-frozen, and stored at −80° C. until use. Collected blood was immediately transferred to sodium citrate tubes and plasma was collected after centrifugation at 2000 g for 15 min. Plasma was stored at−80° C. until use.

### 1.3 Tumor Tissue Processing

Frozen tumors were mixed with 300 μL lysis buffer and 5 μL nuclease in 2 mL screw-cap microcentrifuge tubes. Beads were added and samples were homogenized at a setting of 6 mps, 30 sec/cycle, for a total three cycles with a 30 sec rest on ice between cycles in a Fast-Prep 24 5 G bead beater lysis system (MP Biomedical). Lysed samples were centrifuged at 12,000×g for 10 min at 4° C. Supernatant was removed, aliquoted, snap-frozen, and stored at −80° C. until use.

### 1.4 Anti-PSMA-LP3 Total Antibody and Intact ADC Assays

Anti PSMA-LP3 total antibody and intact ADC assays were performed on a Gyros xPand nanoliter-scale microfluidics immunoassay instrument platform (Gyros Protein Technologies). BioAffy 1000 CDs (Gyros Protein Technologies) containing streptavidin were allowed to equilibrate to room temperature for at least 30 min. Test mouse plasma/tumor lysate samples were minimally diluted 1:10 for plasma and 1:500 for tumor lysate in 2% Tween 20 in Rexxip HN buffer (Gyros Protein Technologies), to bring into quantitative range of assay based on estimated concentration. For both total antibody assay and intact ADC standard curves, anti-PSMA-LP3 ADC was first diluted to 800 ng/mL in Rexxip HN buffer from 50 μg/mL stock, then diluted 2.2-fold 6 times (range of 800 ng/mL-7.06 ng/mL, total of 7 standards). For total antibody assay, capture reagent was biotinylated human PSMA (R&D Systems) at 25 μg/mL in 2% Tween-20 in Rexxip HN buffer, detection reagent was Alexa-Fluor 647-labeled mouse anti-human IgG Fc (CH2 Domain) (BioRad MCA4774) at 25 μg/mL in Rexxip F buffer. For intact ADC assay, capture reagent was biotinylated anti-Compound 1 Fab (2.1A3) fragment 50 μg/mL in 2% Tween-20 in Rexxip HN buffer, detection reagent was Alexa-Fluor 647-labeled mouse anti-human IgG Fc (CH2 Domain) (BioRad MCA4774) at 25 μg/mL in Rexxip F buffer. All samples and standards were centrifuged for 5 min at 5,000×g. The following protocol was then run on the Gyros xPand for both total antibody and intact ADC assays, with a 2 nL/sec flow rate (200 nL volume for washes and reagent treatments):
Needle was washed with Gyros wash buffer pH 11 (Gyros Protein Technologies, P0020096), then PBST
Columns were washed 2× with PBST
Capture reagent was passed over columns
Columns were washed 2× with PBST
Test samples and standards were passed over columns
Columns were washed 2× with PBST
Background was set prior to addition of detection reagent
Detection reagent was passed over columns
Columns were washed 4× with PBST
Total captured signal was detected with 1% PMT setting
Needle was washed with Gyros wash buffer pH 11, then PBST

Collected data was analyzed using the Gyros Evaluator software using a 5-parameter curve fit with response weighting, or using Watson LIMS using a 5% PMT setting and a 5-parameter curve fit with 1/y² weighting for the standard curve. Limit of quantitation (LOQ) for both the total antibody assay and intact ADC assay was 10 ng/mL in neat plasma, with a minimum required plasma dilution (MRD) of 1:10. Pharmacokinetic analyses of collected data was performed using Winnonlin (Certara).

### 1.5 Analysis of Free Payload by Quantitative LC-MS

Methods for analysis of free payload by quantitative LC-MS are described above at Example 9.

### 2 Results

### 2.1 Pharmacokinetics of Anti-PSMA LP3 ADC (Random DAR4) in Non-Tumor-Bearing Mice

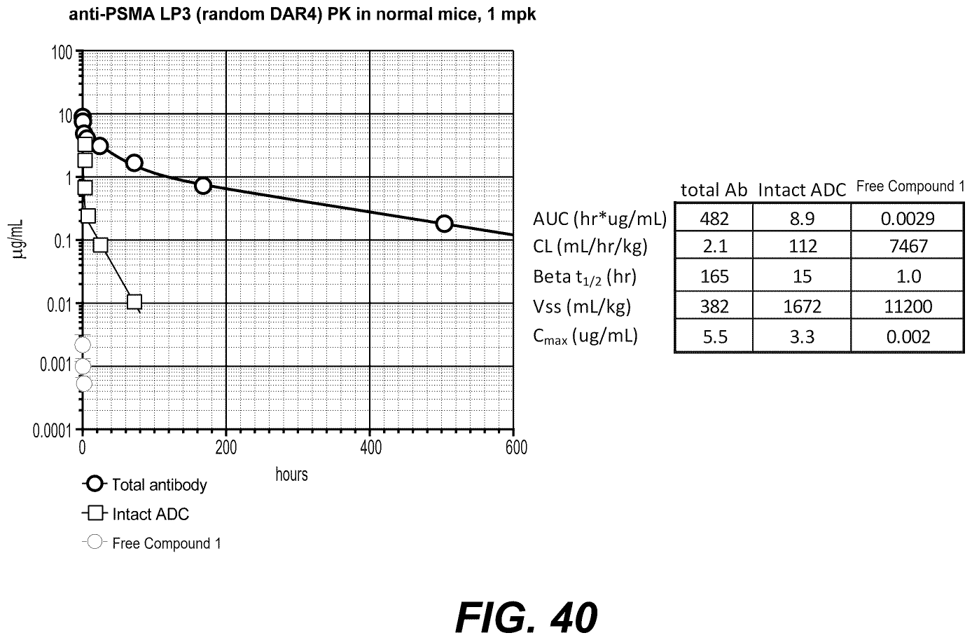

Anti-PSMA LP3 (random DAR4) was administered as a single IV bolus dose to normal nude mice and concentrations of ADC and metabolites were determined using total antibody, intact ADC, and free payload assays. For free Compound 1, only Compound 1 was detected, and no monophosphate forms were found. Intact ADC and total antibody PK parameters were determined using a 2-compartment model, and free Compound 1 was determined using non-compartmental fitting. The results of this analysis are shown in FIG. 40.

The analysis demonstrated instability of the ADC in circulation, as evidenced by the lower AUC and half-life, and increased clearance rate for the intact ADC as compared to the total antibody. This is consistent with plasma stability studies indicating rapid cleavage of Compound 1 linked through the sulfur in the (S) thiophosphate of Compound 1.

### 2.2 Pharmacokinetics of Anti-PSMA LP3 ADC (RESPECT-L DAR2) and Anti-PSMA LP1 (RESPECT-L DAR4) In C4-2 Tumor-Bearing Mice

### 2.2.1 Plasma PK of Anti-PSMA LP3 ADC (RESPECT-L DAR2)

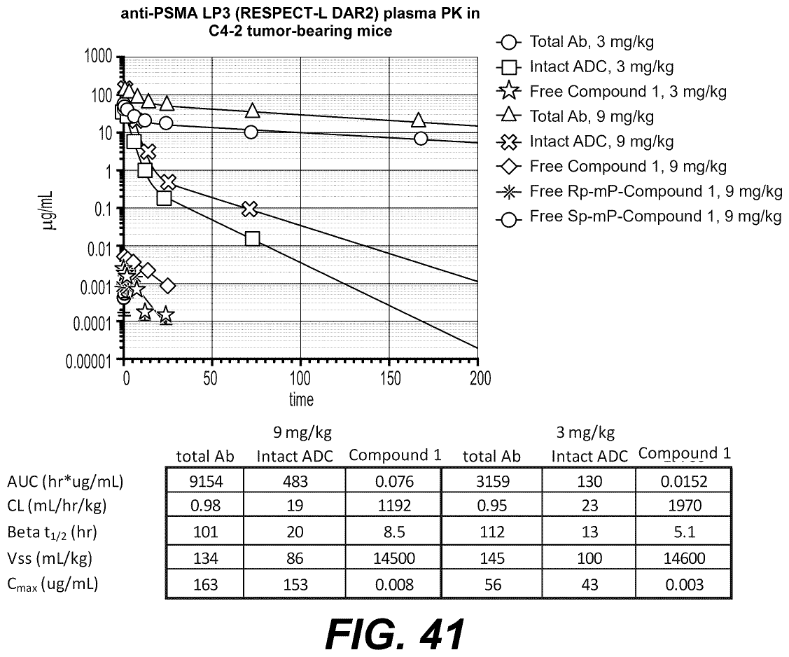

Anti-PSMA LP3 (RESPECT-L DAR2) and anti-PSMA LP1 (RESPECT-L DAR4) were administered as a single IV bolus at 3 mg/kg and 9 mg/kg doses to C4-2 tumor-bearing mice and concentrations of ADC and metabolites were determined using total antibody, intact ADC, and free payload assays for anti-PSMA LP3 (RESPECT-L DAR2). Specificity studies demonstrated that the anti-Compound 1 (anti-payload) reagent antibody 2.1.A3 did not recognize Compound 1 on ADCs where Compound 1 is conjugated through the bridge nitrogen (“N-linked” ADCs), so only free payload was determined for samples from anti-PSMA LP1 (RESPECT-L DAR4)-dosed animals. For free released payload, Compound 1 was the main payload detected, although low levels of Rp- and Sp-monophosphate-Compound 1 were detected in early time points for the animals dosed with 9 mg/kg of anti-PSMA LP3 (RESPECT-L DAR2). Intact ADC and total antibody PK parameters were determined using a 2-compartment model, and free Compound 1 was determined using non-compartmental fitting. The results of this analysis are shown in FIG. 41.

Similar instability of the anti-PSMA LP3 (RESPECT-L DAR2) was observed in the C4-2 tumor bearing mice as was observed for the randomly conjugated DAR4 ADC in non-tumor-bearing animals. A clear dose-response in AUC for total antibody, intact ADC, and released Compound 1 was observed.

### 2.2.2 Intratumoral PK of Anti-PSMA LP3 ADC (RESPECT-L DAR2) and Anti-PSMA LP1 ADC (RESPECT-L DAR4)

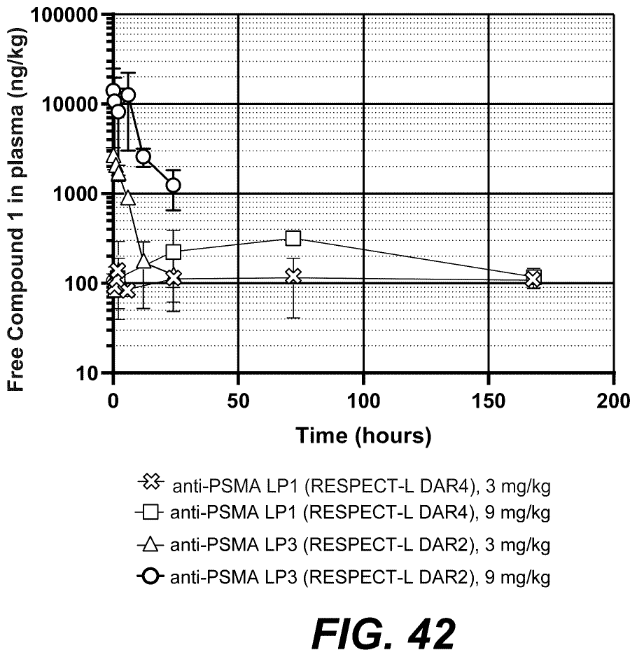

Free Compound 1 concentrations were determined for both anti-PSMA LP3 ADC (RESPECT-L DAR2) and anti-PSMA LP1 ADC (RESPECT-L DAR4) following collection and homogenization of the C4-2 tumors from treated mice. Data is plotted as ng/kg to allow for comparison with tumor uptake. These results are shown in FIG. 42.

Free Compound 1 in plasma was much lower for anti-PSMA LP1 ADC (RESPECT-L DAR4)-dosed animals than for animals dosed with anti-PSMA LP3 ADC (RESPECT-L DAR2), with Cₘₐₓ levels 50-fold lower, even with the increased DAR. This is consistent with the improved in vitro plasma stability of the LP1 ADC compared with LP3-based forms. Cₘₐₓ was also delayed for anti-PSMA LP1 ADC (RESPECT-L DAR4) relative to anti-PSMA LP3 ADC (RESPECT-L DAR2).

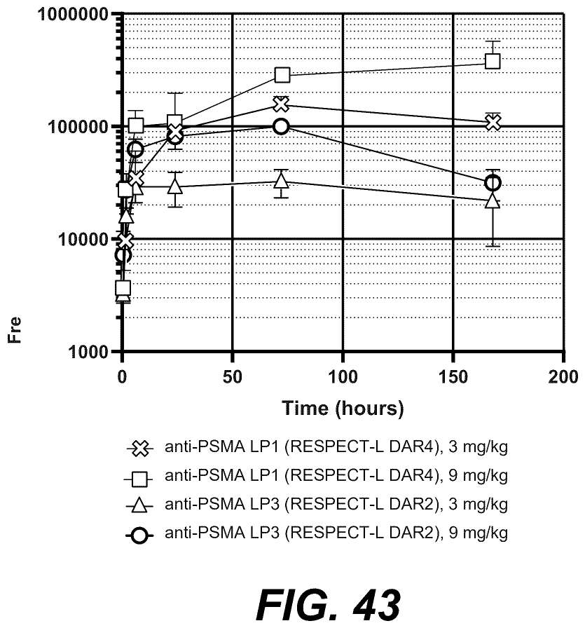

Level of free Compound 1 was also measured in tumor lysates from these same dosed animals. Concentrations were normalized to g tumor weight. These results are shown in FIG. 43.

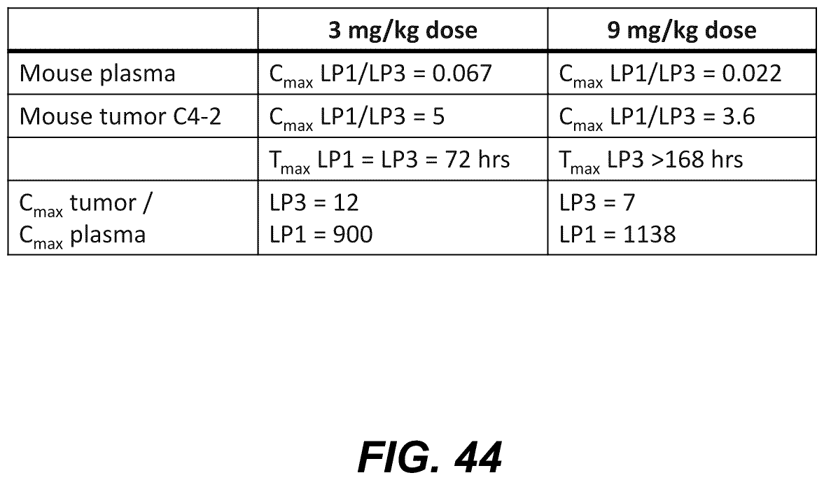

Compound 1 was retained at higher (4 to 5-fold higher Cₘₐₓ) and for a longer time in anti-PSMA LP1 ADC (RESPECT-L DAR4) dosed animals, relative to anti-PSMA LP3 ADC (RESPECT-L DAR2), as well as 3-log greater tumor-to-plasma Cₘₐₓ (FIG. 44).

### Example 14. Payload Release Assay for PSMA-STING ADCs

### 1 Two-step payload release assay method

The LPs for N-linked PSMA-STING ADCs mainly release their payloads in two steps. In the first step, a cleavable amino acid unit is enzymatically cleaved (e.g., by legumain (LP21-23) or cathepsin B (LP1-20, 24, and 27-32)) in acidic condition. The p-aminobenzyl carbamate (pABC) spacer is immediately removed by a first self-immolation reaction. As a result, an intermediate composed of a payload and a second spacer is released. In the second step, the second spacer is removed from the intermediate by a second immolation reaction, and the payload is released. The intermediate is relatively stable in the acidic pH, but the second immolation reaction quickly proceeds in a neutral pH region.

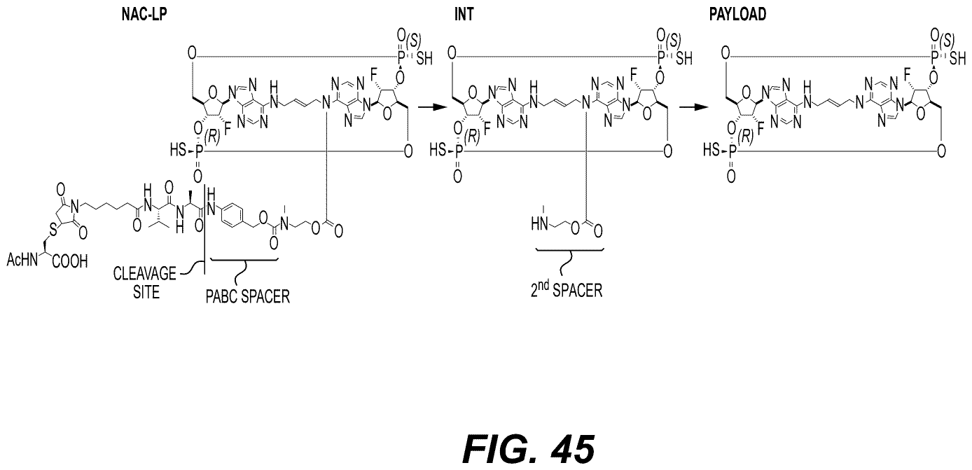

In this assay, the linker-payloads were treated with cathepsin B or legumain at pH 5 to monitor the first step reaction, then the pH of the solution was adjusted to 7 to evaluate the kinetics of the second step reaction. A scheme of payload release assay for representative LP2 is shown in FIG. 45.

### 1.1 Deactivation of Maleimide in LPs

The reactive maleimide moiety was deactivated by conjugating N-acetylcysteine (NAC) before payload release assay. Each LP was treated with 3 molar equivalents of NAC in dimethyl sulfoxide (DMSO) to prepare NAC-LP.

### 1.2 Payload Release Assay

Evaluation of first step reaction (enzymatic trigger cleavage rate). The kinetics for first step reaction for each LP were evaluated by suitable condition C1, C2, or L. The condition applied for each LP is shown in Tables 39, 40, and 41, infra.
Condition C1: Cathepsin B (Sigma-Aldrich, cat #C8571) was diluted to 125 μg/mL in 25 mM sodium acetate buffer pH 5.5 containing 30 mM dithiothreitol (DTT) and 15 mM ethylenediaminetetraacetic acid (EDTA) and activated at room temperature for 10 min. NAC-LP was diluted to 20 μM in 250 μL of 25 mM sodium acetate buffer pH 5.0 containing 20 μM NAC-LP, and 1 mM EDTA. Fifty (50) μL of the solution was taken and 50 μL of methanol containing 1% formic acid was added. The solution was used for the analysis of initial (to) sample. The activated enzyme was added at the final concentration of 1.25 ug/mL to the remaining 200 μL of the NAC-LP solution.
Condition C2: Cathepsin B (Sigma-Aldrich, cat #C8571) was diluted to 125 μg/mL in 20 mM sodium acetate buffer pH 5.5 containing 30 mM DTT and activated at 37° C. for 30 min. NAC-LP was diluted to 20 μM in 250 μL of 20 mM sodium acetate buffer pH 5.0 containing 1 mM DTT. 50 μL of the solution was taken and 50 μL of methanol containing 1% formic acid was added. The solution was used for the analysis of initial (to) sample. The activated enzyme was added at the final concentration of 1.25 pg/mL to the remaining 200 μL of the NAC-LP solution.
Condition L: Legumain (Fisher Scientific 2199-CY-010) was diluted to 47 μg/mL in 20 mM sodium acetate buffer pH 4.0 containing 100 mM NaCl and activated at 37° C. for 30 min. NAC-LP was diluted to 20 μM in 250 μL of 20 mM sodium acetate buffer pH 5.0 containing 1 mM DTT. 50 μL of the solution was taken and 50 μL of methanol containing 1% formic acid was added. The solution was used for the analysis of initial (to) sample. The activated enzyme was added at the final concentration of 1.2 μg/mL to the remaining 200 μL of the NAC-LP solution.

The sampling schedule was common for each assay condition. 30 μL of the solution was sampled at t=15 min, 30 min, and 240 min (t₁₅, t₃₀, and t₂₄₀, respectively) and 30 μL of methanol containing 1% formic acid was added to stop the reaction. These solutions were analyzed by HPLC.

The remaining solution was used as t′₀ solution for the evaluation of the second step reaction.

### 1.3 Payload Release Assay: Evaluation of Second Step Reaction (Self-Immolation Rate of Second Spacers)

The procedures were common regardless of the assay condition for the first step reaction. After the first step reaction (sampling of t₂₄₀ solution), 50 μL of 200 mM phosphate buffer pH 7.0 was immediately added to 50 μL of t′₀ solution to initiate the second step reaction. At t′=15 min and 30 min (t′₁₅ and t′₃₀, respectively), 30 μL of the solution was sampled and 30 μL of methanol containing 1% formic acid was added to stop the reaction. These solutions were analyzed by HPLC.

### 1.4 HPLC Conditions

### For Assay Condition C1:

System: Waters Acquity UPLC
Column: Waters Acquity CSH C18, 2.1×50 mm, particle size 1.7 um
Detection: UV260 nm
Flow rate: 0.5 mL/min
Mobile phase A: a mixture of water and ammonium formate (1000/1, w/v)
Mobile phase B: a mixture of water, acetonitrile, and ammonium formate (100/900/1, v/v/w)
Gradient program (t, B conc): (0 to 0.2 min, 5%)−(0.2 to 1.5 min, 5% to 60%)−(1.5 to 1.8 min, 60% to 95%)−(1.8 to 2.0 min, 95%)−(2.01 to 2.5 min, 5%)
Column temperature: 40° C.
Sample temperature: 5° C.
Injection volume: 5 μL

### For Assay Conditions C2 and L:

System: Waters Acquity UPLC
Column: Waters Acquity CSH C18, 2.1×50 mm, particle size 1.7 um
Detection: UV260 nm
Flow rate: 0.3 mL/min
Mobile phase A: a mixture of water and ammonium formate (1000/1, w/v)
Mobile phase B: a mixture of water, acetonitrile, and ammonium formate (100/900/1, v/v/w)
Gradient program (t, B conc): (0 to 0.2 min, 5%)−(0.2 to 1.8 min, 5% to 60%)−(1.8 to 2.2 min, 60% to 95%)−(2.21 to 3 min, 5%)
Column temperature: 40° C.
Sample temperature: 15° C.
Injection volume: 5 μL

### 1.5 Data Analysis

Kinetic analysis for the first step reaction was conducted as follows.

$$Residual ratio of LP = \frac{{A}_{LP}}{{A}_{LP} + {A}_{INT} + {A}_{P}}$$

$$Rate constant ( k , {min}^{- 1} ) = - \frac{1}{t} ln \frac{Residual ratio of LP at t}{Residual ratio of LP at {t}_{0}}$$

The rate constant k was evaluated for t₁₅ and t₃₀, and their average was used as a rate constant for the first step reaction (enzymatic trigger cleavage rate).

Kinetic analysis for the second step reaction was conducted as follows.

$$Residual ratio of INT = \frac{{A}_{INT}}{{A}_{INT} + {A}_{P}}$$

$$Rate constant ( {k}^{′} , {min}^{- 1} ) = - \frac{1}{{t}^{′}} ln \frac{Residual ratio of INT at {t}^{′}}{Residual ratio of INT at {t}_{0}^{′}}$$

The rate constant k′ was evaluated for t′₁₅ and t′₃₀, and their average was used as a rate constant for the 2nd step reaction (self-immolation rate of 2^{nd} spacer). The residual ratio at t′₀ is calculated from each peak area in the t₂₄₀ sample.
A_{LP}: HPLC peak area of NAC-LP
A_{INT}: HPLC peak area of INT
Aₚ: HPLC peak area of payload
t: sampling time (min) in the first step reaction
t′: sampling time (min) in the second step reaction (t′₀=t₂₄₀=the time point when pH was adjusted to 7 by adding 200 mM phosphate buffer)

### 2 Results

30 LPs were evaluated in the assay. The results are summarized in Tables 39 and 40.

LPs listed in Table 39 were evaluated in assay method C1 and those in Table 40 were evaluated by method C2 or L (method L was applied only to LP21-23 which have legumain cleavable triggers). The results for LP2 indicated that the slight difference between assay methods C1 and C2 had little impact on the first and second step reactions.

TABLE 39
Summary of payload release rate (1)

| | | | | Rate constant | | Rate constant | |
|---|---|---|---|---|---|---|---|
| | Spacer | | | for 1st step | | for 2nd step | |
| | (maleimide- | Trigger | | reaction | Relative | reaction | Relative |
| LP# | trigger) | sequence | 2nd spacer | (k, min − 1) | k value ^{b} | (k′, min − 1) | k′ value ^{c} |
| 1 | C6 | VA | MEC | 1.87E−03 | 1.4 | 1.60E−02 | 0.8 |
| 2 | | | | 1.33E−03 | 1.0 | 1.99E−02 | 1 |
| 3 | | | No spacer | 6.97E−03 | 5.3 | NA ^{d} | NA ^{d} |
| 4 | | | cPrCH2carbonyl | 2.05E−03 | 1.5 | 1.01E−02 | 0.5 |
| 5 | | | MEA | 2.27E−03 | 1.7 | 1.60E−03 | 0.1 |
| 6 | | | cPrMEC | 1.72E−03 | 1.3 | 1.38E−02 | 0.7 |
| 7 | | | DMMEC | 1.98E−03 | 1.5 | 2.03E−02 | 1.0 |
| 8 | | | b-cPrMEC | 1.41E−03 | 1.1 | 2.67E−02 | 1.3 |
| 9 | | | No spacer | 9.05E−03 | 6.8 | NA ^{d} | NA ^{d} |
| 10 | | | HEC | 2.45E−03 | 1.9 | 1.78E−02 | 0.9 |
| 11 | mPEG₂- | | MEC | 1.26E−03 | 1.0 | 2.02E−02 | 1.0 |
| | Lys(e-PEG₈- | | | | | | |
| | OMe)-PEG₂ | | | | | | |
| 12 | PEG₈ | | | 5.00E−04 | 0.4 | 1.98E−02 | 1.0 |
| 13 | PEG2 | | | 2.01E−03 | 1.5 | 1.90E−02 | 1.0 |
| 14 | C6 | VC | | 8.87E−03 | 6.7 | 1.96E−02 | 1.0 |
| 15 | | VK | | 1.26E−02 | 9.5 | 1.94E−02 | 1.0 |
| 16 | | VA | Bz | 3.43E−03 | 2.6 | Rapid | Rapid |
| | | | | | | conversion | conversion |
| | | | | | | to payload | to payload |
| | | | | | | at pH 5 ^{e} | at pH 5 ^{e} |
| 20 | | | (L)-prolinol | 2.88E−03 | 2.2 | 1.15E−01 | 5.8 |
| 28 | | | F2-(L)-prolinol | 2.86E−03 | 2.2 | Rapid | Rapid |
| | | | | | | conversion | conversion |
| | | | | | | to payload | to payload |
| | | | | | | at pH 5 ^{e} | at pH 5 ^{e} |
| 30 | | | MEC | 8.11E−04 | 0.6 | 2.67E−02 | 1.3 |

^{a}LPs listed in this table were all cathepsin cleavable and evaluated by assay condition C1.
^{b}The relative k value of each LP normalized by k of LP2.
^{c}The relative k′ value of each LP normalized by k′ of LP2.
^{d}The LPs had no 2nd spacer.
^{e}The 2nd spacers were removed too quickly even at pH 5 (during 1st reaction step) and the self-immolation rate were higher than the upper limit of this assay method.

TABLE 40
Summary of payload release rate (2)

| | | | | Rate constant | | Rate constant | |
|---|---|---|---|---|---|---|---|
| | Spacer | | | for 1st step | | for 2nd step | |
| | (maleimide- | Trigger | | reaction | Relative | reaction | Relative |
| LP# | trigger) | sequence | 2nd spacer | (k, min − 1) | k value ^{b} | (k′, min − 1) | k′ value ^{c} |
| 2 | C6 | VA | MEC | 1.63E−03 | 1.0 | 2.29E−02 | 1.0 |
| 17 | C2 | | Bz | 5.43E−03 | 3.3 | Rapid | Rapid |
| | | | | | | conversion | conversion |
| | | | | | | to payload | to payload |
| | | | | | | at pH 5 ^{d} | at pH 5 ^{d} |
| 18 | | | (L)-prolinol | 4.35E−03 | 2.7 | 1.36E−01 | 6.0 |
| 19 | PEG2 | | | 3.18E−03 | 1.9 | 1.26E−01 | 5.5 |
| 21 | C6 | AAN | | 1.61E−01 | 1.0^{e} | 1.40E−01 | 6.1 |
| 22 | | A(NMe)AN | | 1.09E−01 | 0.7^{e} | 1.38E−01 | 6.0 |
| 23 | | N | | 8.60E−03 | 0.1^{e} | 1.71E−01 | 7.5 |
| 24 | | GGFG | | 5.64E−04 | 0.3 | 1.28E−01 | 5.6 |
| 27 | C2 | VA | F2-(L)-prolinol | 5.50E−03 | 3.4 | Rapid | Rapid |
| | | | | | | conversion | conversion |
| | | | | | | to payload | to payload |
| | | | | | | at pH 5 ^{d} | at pH 5 ^{d} |
| 29 | C6 | | (D)-prolinol | 6.12E−03 | 3.8 | 4.70E−02 | 2.1 |
| 31 | | | MEC | 3.72E−03 | 2.3 | 4.78E−02 | 2.1 |
| 32 | | | | 2.00E−03 | 1.2 | 5.88E−02 | 2.6 |

^{a}LPs 21-23 had legumain cleavable trigger and were evaluated by method L. The others had cathepsin B cleavable trigger and were evaluated by method C2.
^{b}The relative k value of each LP normalized by k of LP2 (except for LP21-23, see foot note e below). A High k value indicates high trigger cleavage rate.
^{c}The relative k′ value of each LP normalized by k′ of LP2. A high k′ value indicates high 2^{nd} self-immolation rate.
^{d}The 2nd spacers were removed too quickly even at pH 5 (during 1st reaction step) and the self-immolation rate were higher than the upper limit of this assay method.
^{e}The relative k value of each LP normalized by k of LP21.

TABLE 41
Summary of payload release rate (3)

| | | | | Rate constant | | Rate constant | |
|---|---|---|---|---|---|---|---|
| | Spacer | | | for 1st step | | for 2nd step | |
| | (maleimide- | Trigger | | reaction | Relative | reaction | Relative |
| LP# | trigger) | sequence | 2nd spacer | (k, min − 1) | k value ^{b} | (k′, min − 1) | k′ value ^{c} |
| 2 | C6 | VA | MEC | 1.40E−03 | 1.0 | 2.68E−02 | 1.0 |
| 25 | C2P2 | | (L)-prolinol | 9.51E−04 | 0.7 | 1.35E−01 | 5.0 |
| 26 | | | F2-(L)-prolinol | 1.69E−03 | 1.2 | Rapid | Rapid |
| | | | | | | conversion | conversion |
| | | | | | | to payload | to payload |
| | | | | | | at pH 5 ^{d} | at pH 5 ^{d} |

^{a}LPs listed in this table were all cathepsin cleavable and evaluated by assay condition C2.
^{b}The relative k value of each LP normalized by k of LP2. A High k value indicates high trigger cleavage rate.
^{c}The relative k′ value of each LP normalized by k′ of LP2. A high k′ value indicates high 2^{nd} self-immolation rate.
^{d}The 2^{nd} spacers were removed too quickly even at pH 5 (during 1st reaction step) and the self-immolation rate were higher than the upper limit of this assay method.

The trigger cleavage rate was mainly dependent on the trigger peptide sequence, and may also have been influenced by second spacers. Regarding the second spacers, a high self-immolation rate, which can be an important attribute for higher potency of ADC, was observed for benzoate (LP16 and LP17), di-F-(L)-prolinol (LP26, LP27, and LP28), and (L)-prolinol (LP18-25) spacers.

The structure of spacer (between maleimide and trigger) and trigger had little impact on the rate of second self-immolation.

### Example 15. In vitro interferon-6 (IFN-β) Release Assay for PSMA-STING ADCs

### 1 Methods

LP1-32, 39, 42-48, 50 and 53 were tested in this example.

### 1.1 In Vitro IFN-β Release Assay

Upon STING activation, the modified STING recruits TBK1 and IRF3 genes and ultimately regulates the expression and secretion of pro-inflammatory cytokines including IFN-(3.

IFN-β release was measured for in vitro potency of the indicated PSMA-STING ADCs. The PSMA positive prostate cancer cell line (C4-2) and a human leukemia monocytic cell line (THP-1), which has been extensively used to study monocyte/macrophage functions, were used for the assay. The ADCs were tested both in the co-culture of C4-2 and THP-1 cells, and in monoculture of THP-1. The IFN-β release from co-culture represented the potency of ADCs, and the IFN-β release from monoculcure represented the ADC's effect on immune cells only (off-target effect).

1.1.1 In Vitro IFN-6 Release from C4-2 and THP-1 Co-Culture

C4-2 cells were seeded in 96-well flat bottom culture plates at 15,000 cells/well in 100 μL of cell culture medium (complete RPMI-10% FBS) and incubated overnight at 37° C., 5% CO₂. The next day, 50 μL/well of ADC reagents were added and serially diluted to from 0.01-100 nM, followed by the addition of THP-1 cells at 15,000 cells/well in 50 μL/well of cell culture medium, then mixed well and incubated at 37° C., 5% CO₂ for 20-22 hours. After incubation, culture plates were centrifuged at 1500 rpm for 5 minutes, then supernatant was collected and stored at −80° C. prior to measurement of human IFN-β.

1.1.2 In Vitro IFN-6 Release from THP-1 Monoculture

THP-1 cells were seeded in 96-well flat bottom culture plates at 30,000 cells/well in 100 μL of cell culture medium (complete RPMI-10% FEBS). 100 μL/well of ADC reagents were then added, serially diluted from 0.01-100 nM, mixed well, then incubated at 37° C., 5% CO₂ for 20-22 hours. After incubation, culture plates were centrifuged at 1500 rpm for 5 minutes, supernatant was collected, stored at −80° C. prior to measurement of human IFN-β.

1.1.3 Human IFN-β measurement by ELISA

Human IFN-β was measured using Human IFN-β DuoSet ELISA (R&D systems, cat #DY814-05) following the manufacturer's instruction except for an expanded standard range of 7.8-1,000 pg/ml. At the end of the assay, QuantaBlu Fluorogenic peroxidase substrate (Thermo, cat #15169) was used to probe the signal. Plates were read on a BMG Labtech Clariostar plate reader at excitation 320 nM and emission of 405 nM. IFN-β was quantified against the IFN-beta standard curve (7.8-1,000 μg/ml), and the data plot was generated using GraphPad Prism.

### 2 Results

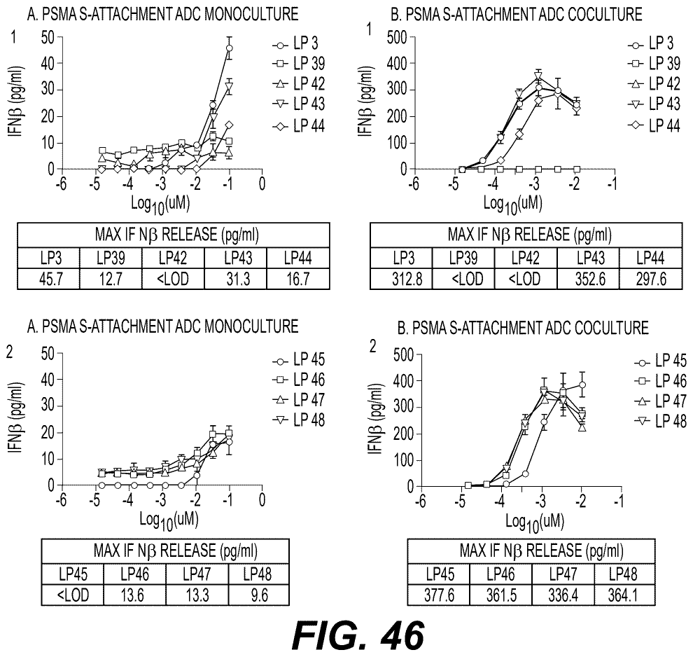

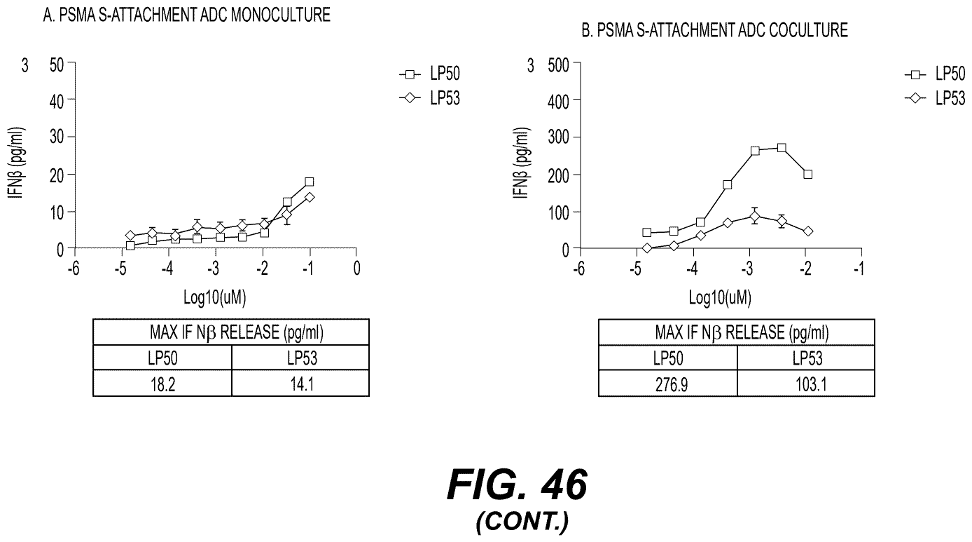

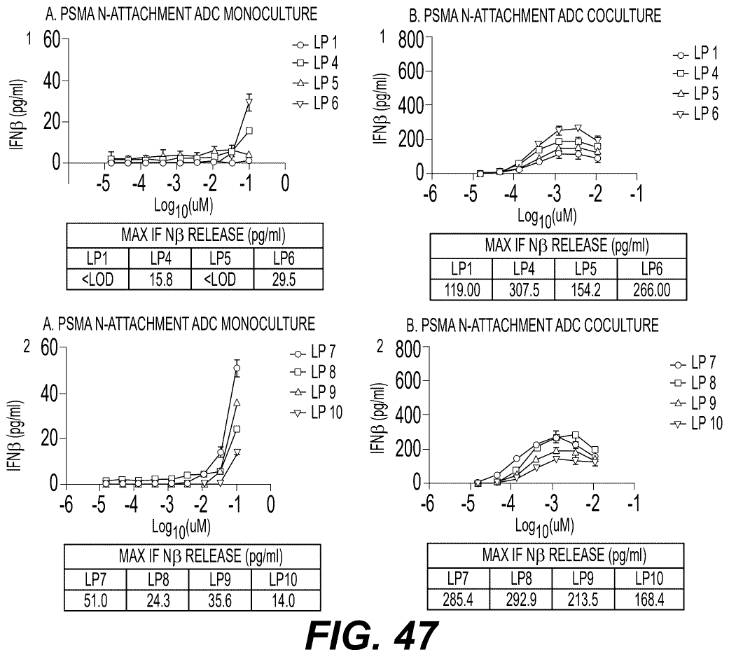

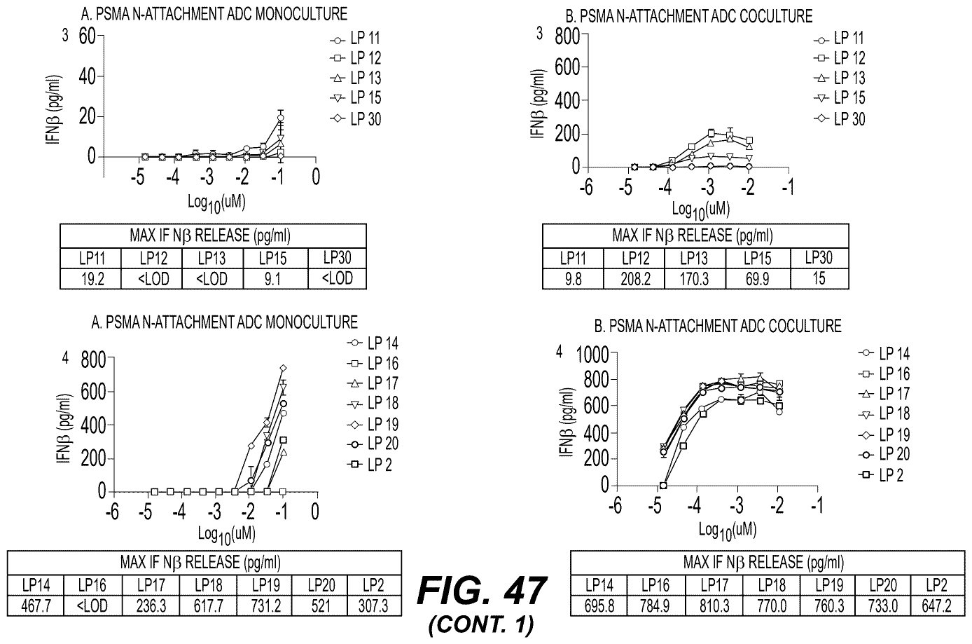

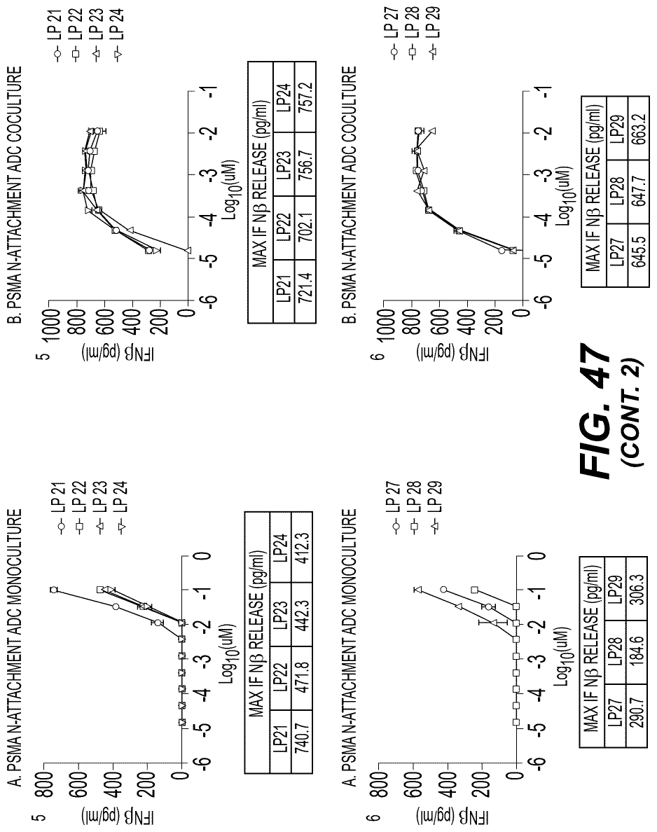

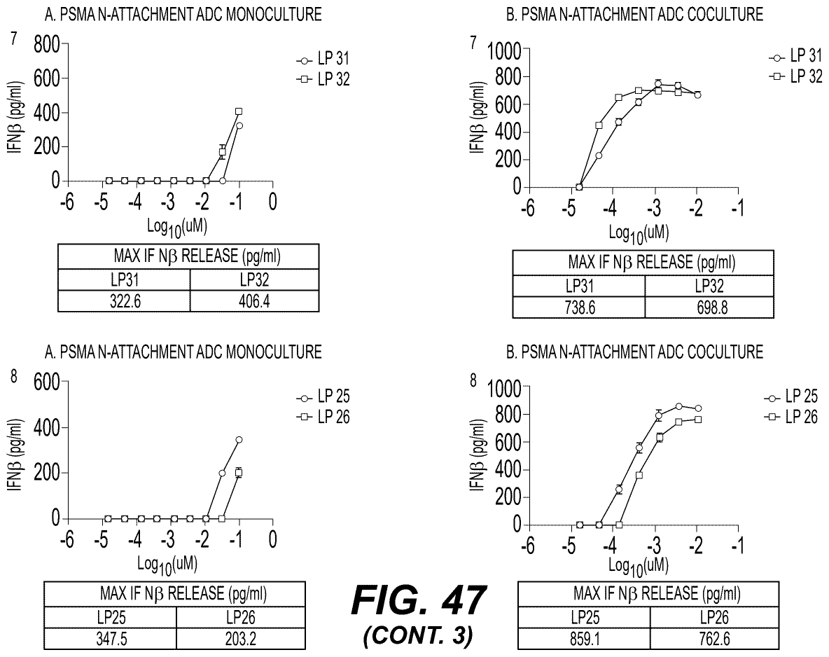

A total of 42 ADCs were evaluated. The quantified IFN-β is shown for both monoculture and co-culture. The ADCs with S-attachment (SP) and N-attachment (SN) are shown in FIGS. 46 and 47. The maximal IFN-β release is also shown. Samples with detection below the limit of detection are shown as “<LOD.”

In general, N-attachment ADCs showed higher levels of IFN-β release compared with S-attachment ADCs. Other changes to the linker design impacted IFN-β release.

Selected Sequences

| SEQ ID NO | |
|---|---|
| 67 | MWNLLHETDSAVATARRPRWLCAGALVLAGGFFLLGFLFGW |
| (human PSMA) | FIKSSNEATNITPKHNMKAFLDELKAENIKKFLYNFTQIPH |
| | LAGTEQNFQLAKQIQSQWKEFGLDSVELAHYDVLLSYPNKT |
| | HPNYISIINEDGNEIFNTSLFEPPPPGYENVSDIVPPESAF |
| | SPQGMPEGDLVYVNYARTEDFFKLERDMKINCSGKIVIARY |
| | GKVFRGNKVKNAQLAGAKGVILYSDPADYFAPGVKSYPDGW |
| | NLPGGGVQRGNILNLNGAGDPLTPGYPANEYAYRRGIAEAV |
| | GLPSIPVHPIGYYDAQKLLEKMGGSAPPDSSWRGSLKVPYN |
| | VGPGFTGNFSTQKVKMHIHSTNEVTRIYNVIGTLRGAVEPD |
| | RYVILGGHRDSWVFGGIDPQSGAAVVHEIVRSFGTLKKEGW |
| | RPRRTILFASWDAEEFGLLGSTEWAEENSRLLQERGVAYIN |
| | ADSSIEGNYTLRVDCTPLMYSLVHNLTKELKSPDEGFEGKS |
| | LYESWTKKSPSPEFSGMPRISKLGSGNDFEVFFQRLGIASG |
| | RARYTKNWETNKFSGYPLYHSVYETYELVEKFYDPMFKYHL |
| | TVAQVRGGMVFELANSIVLPFDCRDYAVVLRKYADKIYSIS |
| | MKHPQEMKTYSVSFDSLESAVKNFTEIASKESERLQDFDKS |
| | NPIVLRMMNDQLMFLERAFIDPLGLPDRPFYRHVIYAPSSH |
| | NKYAGESFPGIYDALFDIESKVDPSKAWGEVKRQIYVAAFT |
| | VQAAAETLSEVA |
| 68 | MPHSSLHPSIPCPRGHGAQKAALVLLSACLVTLWGLGEPPE |
| (human STING) | HTLRYLVLHLASLQLGLLLNGVCSLAEELRHIHSRYRGSYW |
| | RTVRACLGCPLRRGALLLLSIYFYYSLPNAVGPPFTWMLAL |
| | LGLSQALNILLGLKGLAPAEISAVCEKGNFNVAHGLAWSYY |
| | IGYLRLILPELQARIRTYNQHYNNLLRGAVSQRLYILLPLD |
| | CGVPDNLSMADPNIRFLDKLPQQTGDHAGIKDRVYSNSIYE |
| | LLENGQRAGTCVLEYATPLQTLFAMSQYSQAGFSREDRLEQ |
| | AKLFCRTLEDILADAPESQNNCRLIAYQEPADDSSFSLSQE |
| | VLRHLRQEEKEEVTVGSLKTSAVPSTSTMSQEPELLISGME |
| | KPLPLRTDFS |
| 69 | TCPPCPAPELLGGPSVFLFPPKPKDTLMISRTPEVTCVVVD |
| (human wildtype IgG1 Fc) | VSHEDPEVKFNWYVDGVEVHNAKTKPREEQYNSTYRVVSVL |
| | TVLHQDWLNGKEYKCKVSNKALPAPIEKTISKAKGQPREPQ |
| | VYTLPPSRDELTKNQVSLTCLVKGFYPSDIAVEWESNGQPE |
| | NNYKTTPPVLDSDGSFFLYSKLTVDKSRWQQGNVFSCSVMH |
| | EALHNHYTQKSLSLSPGK |
| 70 | GGPSVFLFPPKPKDTLMISRTPEVTCVVVDVSHEDPEVKFN |
| (human IgG1 CH2 domain) | WYVDGVEVHNAKTKPREEQYNSTYRVVSVLTVLHQDWLNGK |
| | EYKCKVSNKALPAPIEKTISKAK |
| 71 | GQPREPQVYTLPPSRDELTKNQVSLTCLVKGFYPSDIAVEW |
| (human IgG1 CH3 domain) | ESNGQPENNYKTTPPVLDSDGSFFLYSKLTVDKSRWQQGNV |
| | FSCSVMHEALHNHYTQKSLSLSPGK |

1. A humanized anti-prostate-specific membrane antigen (PSMA) antibody or antigen-binding fragment thereof, wherein the antibody or antigen-binding fragment binds specifically to human PSMA, and wherein the antibody or antigen-binding fragment comprises
(i) three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or
(ii) three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 33 (LCDR1), SEQ ID NO: 36 (LCDR2), and SEQ ID NO: 37 (LCDR3), as defined by the Kabat numbering system; or
(iii) three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

2. The anti-PSMA antibody or antigen-binding fragment of claim 1, wherein the antibody or antigen-binding fragment comprises
(i) three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or
(ii) three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system.

3. The anti-PSMA antibody or antigen-binding fragment of claim 1, wherein the antibody or antigen-binding fragment comprises
(i) a heavy chain variable region comprising an amino acid sequence of SEQ ID NO: 1, and a light chain variable region comprising an amino acid sequence of SEQ ID NO: 15; or
(ii) a heavy chain variable region comprising an amino acid sequence of SEQ ID NO: 2, and a light chain variable region comprising an amino acid sequence of SEQ ID NO: 15; or
(iii) a heavy chain variable region comprising an amino acid sequence of SEQ ID NO: 3, and a light chain variable region comprising an amino acid sequence of SEQ ID NO: 15; or
(iv) a heavy chain variable region comprising an amino acid sequence of SEQ ID NO: 14, and a light chain variable region comprising an amino acid sequence of SEQ ID NO: 15; or
(v) a heavy chain variable region comprising an amino acid sequence of SEQ ID NO: 14, and a light chain variable region comprising an amino acid sequence of SEQ ID NO: 19.

4. The anti-PSMA antibody or antigen-binding fragment of any one of claims 1 to 3, wherein the antibody or antigen-binding fragment comprises a heavy chain variable region comprising an amino acid sequence of SEQ ID NO: 14, and a light chain variable region comprising an amino acid sequence of SEQ ID NO: 19.

5. The anti-PSMA antibody or antigen-binding fragment of any one of claims 1 to 4, wherein the antibody or antigen-binding fragment comprises a human IgG heavy chain constant region.

6. The anti-PSMA antibody or antigen-binding fragment of any one of claims 1 to 5, wherein the antibody or antigen-binding fragment comprises a human IgG1 heavy chain constant region.

7. The anti-PSMA antibody or antigen-binding fragment of any one of claims 1 to 6, wherein the antibody or antigen-binding fragment comprises a human Ig kappa light chain constant region.

8. The anti-PSMA antibody or antigen-binding fragment of any one of claims 1 to 4, wherein the antigen-binding fragment has a melting temperature (Tm)>80° C., wherein optionally the antigen-binding fragment is a Fab.

9. The anti-PSMA antibody or antigen-binding fragment of any one of claims 1 to 8, wherein the antibody or antigen-binding fragment is attached to at least one linker, wherein optionally the at least one linker is cleavable.

10. The anti-PSMA antibody or antigen-binding fragment of claim 9, wherein the at least one linker is conjugated to a cytotoxic agent or detectable reagent.

11. A linker-payload conjugate comprising L-D, wherein L is a linker that covalently attaches to D, wherein D comprises a compound according to one of the following Formulae: 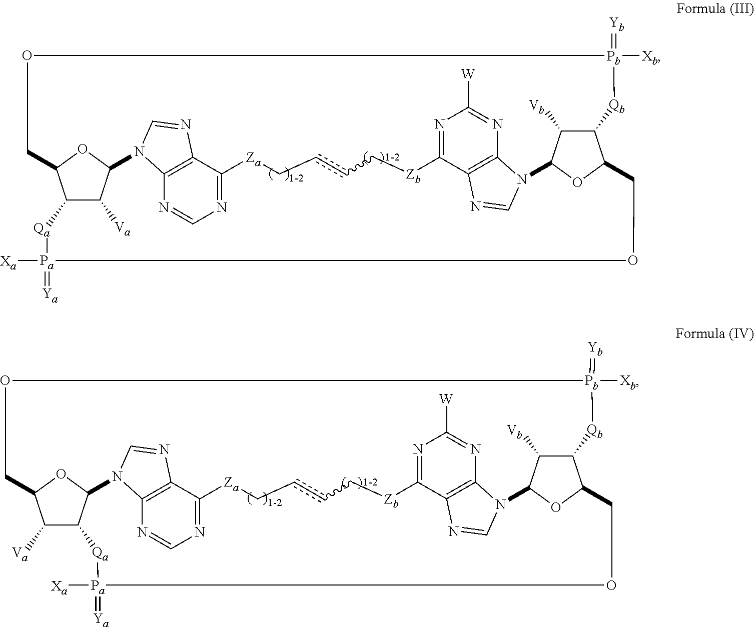
an isomer thereof, a deuterated derivative of the compound or isomer; or a salt of the compound, isomer, or deuterated derivative;
wherein, independently for each occurrence,
each of Pₐ and P_{b}, when not racemic, is independently selected from (R)-stereochemistry and (S)-stereochemistry;
each of Qₐ and Q_{b} is independently selected from NH and O;
each of Vₐ and V_{b} is independently selected from F and OH;
W is selected from H and NH₂;
each of Xₐ and X_{b} is independently selected from OH and SH;
each of Yₐ and Y_{b} is independently selected from O and S;
each Zₐ and Z_{b} is independently selected from CH₂, O, and NH; and [symbol]
means that the bond is selected from a single bond ( [symbol] ), a double bond ( [symbol] ) of (E)- or (Z)-configuration, or a triple bond ( [symbol] );
provided that at least one of Zₐ and Z_{b} is NH or at least one of Xₐ and X_{b} is SH.

12. The linker-payload conjugate of claim 11, wherein Pₐ is (S)-configuration and P_{b} is (R)-configuration.

13. The linker-payload conjugate of claim 11, wherein Pₐ is (R)-configuration and P_{b} is (R)-configuration.

14. The linker-payload conjugate of any one of claims 11 to 13, wherein Qₐ and Q_{b} are O.

15. The linker-payload conjugate of any one of claims 11 to 14, wherein Vₐ and V_{b} are OH.

16. The linker-payload conjugate of any one of claims 11 to 14, wherein Vₐ and V_{b} are F.

17. The linker-payload conjugate of any one of claims 11 to 16, wherein W is H.

18. The linker-payload conjugate of any one of claims 11 to 17, wherein at least one of Zₐ and Z_{b} is NH.

19. The linker-payload conjugate of any one of claims 11 to 18, wherein Zₐ and Z_{b} are NH.

20. The linker-payload conjugate of any one of claims 11 to 19, wherein [symbol] comprises a double bond ( [symbol] ) of (E)- or (Z)-configuration.

21. The linker-payload conjugate of any one of claims 11 to 19, wherein the bridge 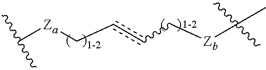
has the structure H or

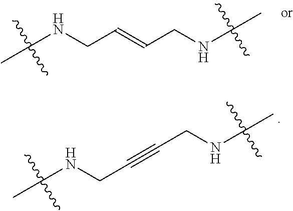 [SMILES: *NC/C=C/CN* ; *NCC#CCN*]

22. The linker-payload conjugate of any one of claims 11 to 21, wherein at least one of Yₐ and Y_{b} is O.

23. The linker-payload conjugate of any one of claims 11 to 22, wherein Yₐ and Y_{b} are O.

24. The linker-payload conjugate of any one of claims 11 to 23, wherein at least one of Xₐ and X_{b} is SH.

25. The linker-payload conjugate of any one of claims 11 to 24, wherein Xₐ and X_{b} are SH.

26. The linker-payload conjugate of any one of claims 11 to 25, wherein D comprises a compound of Formula (III).

27. The linker-payload conjugate of claim 11, wherein D comprises a compound of Formula (III) selected from: 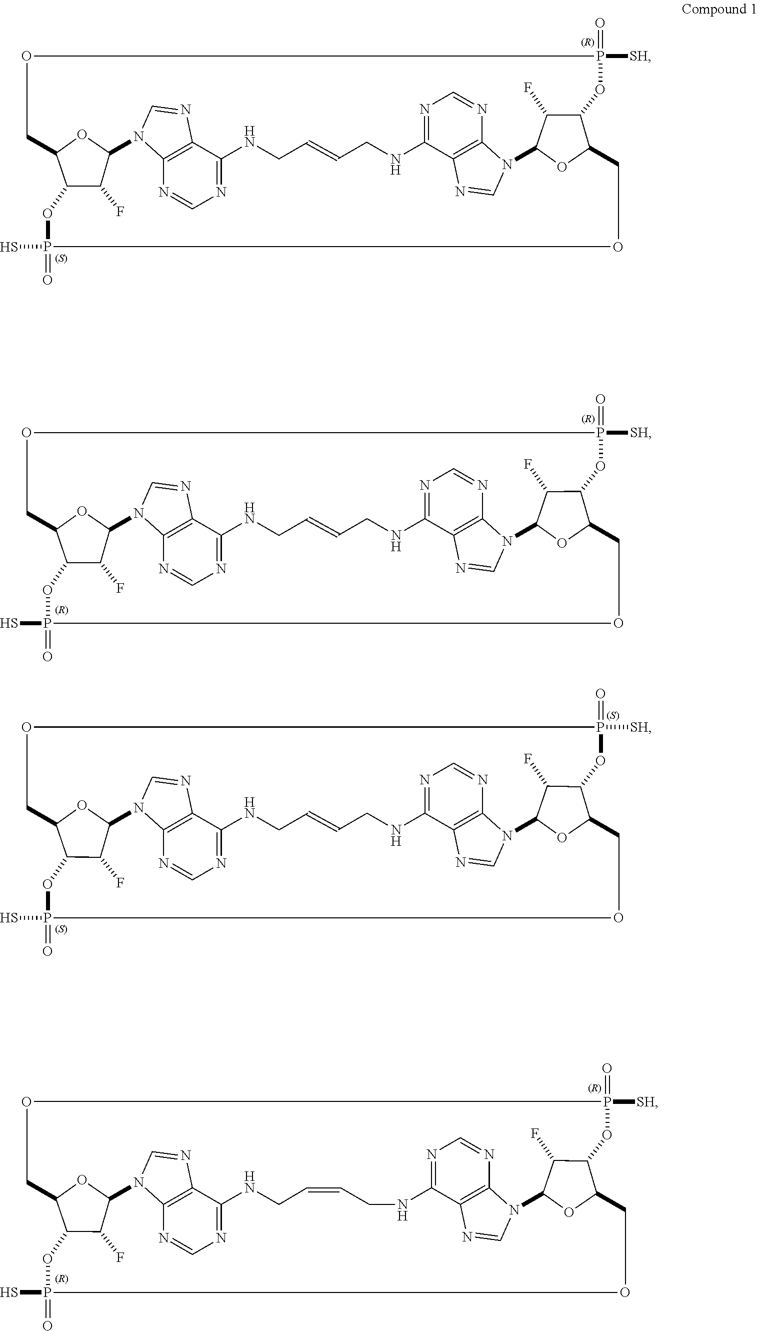 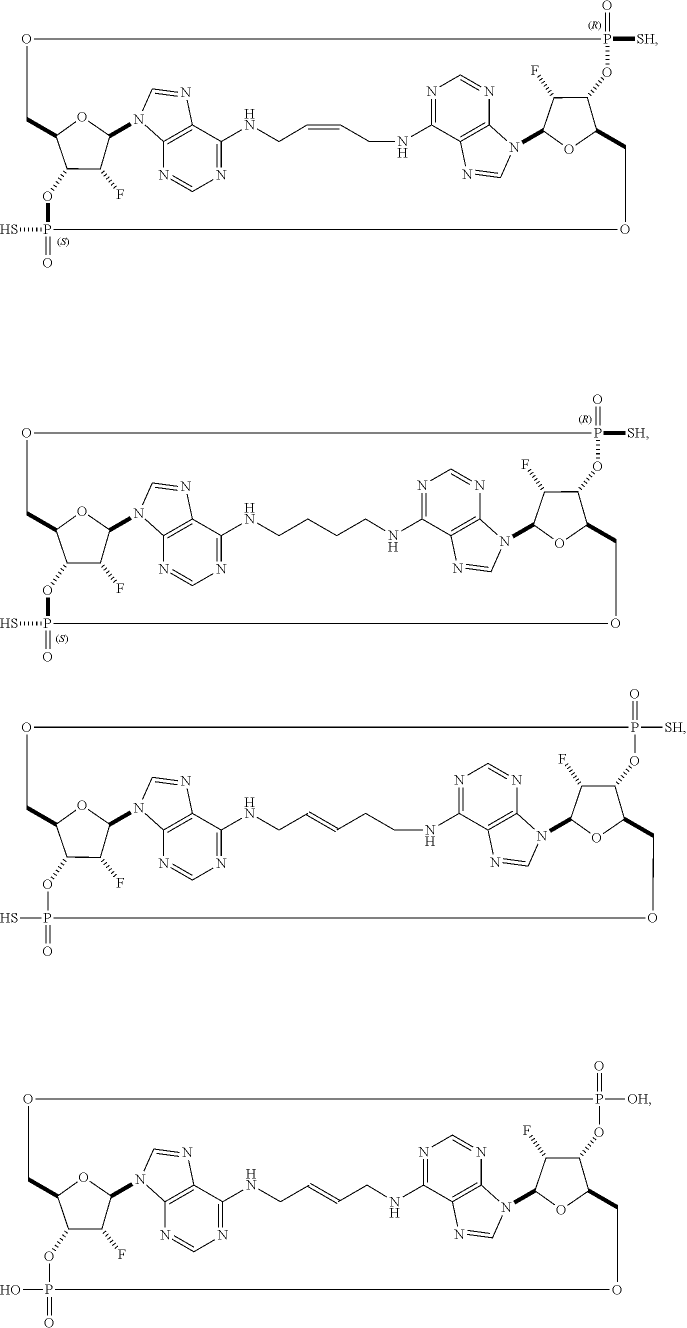 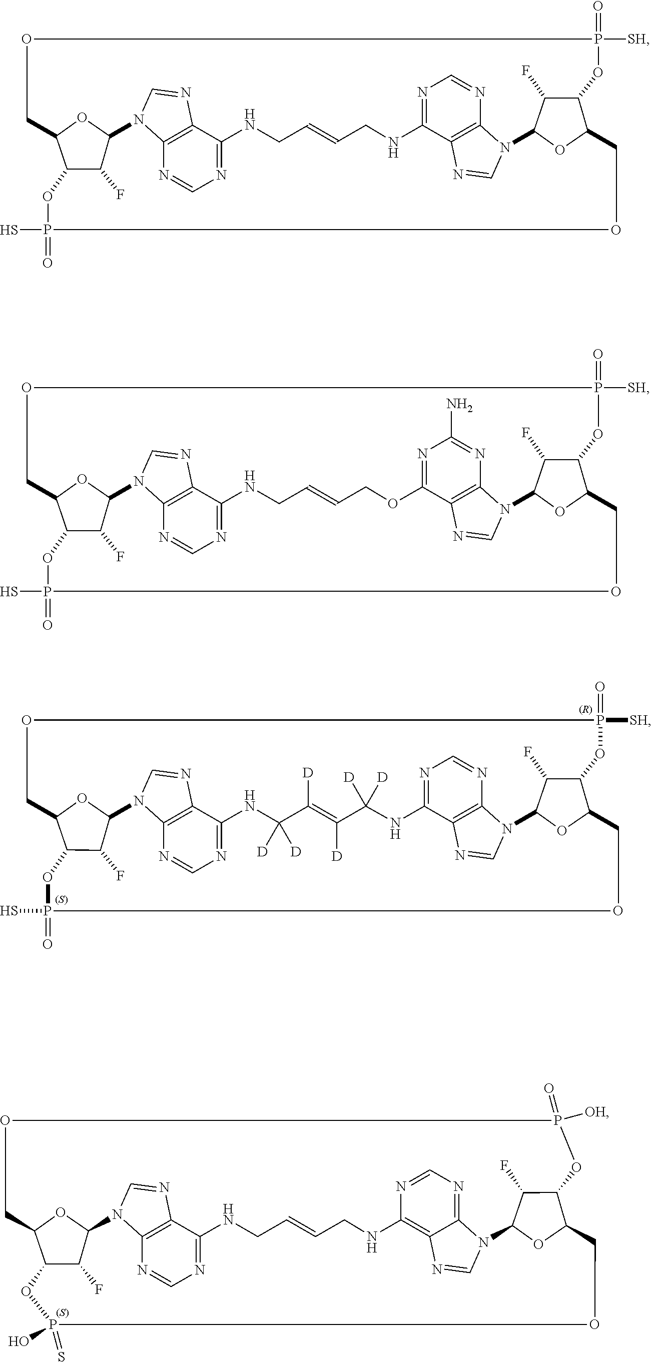 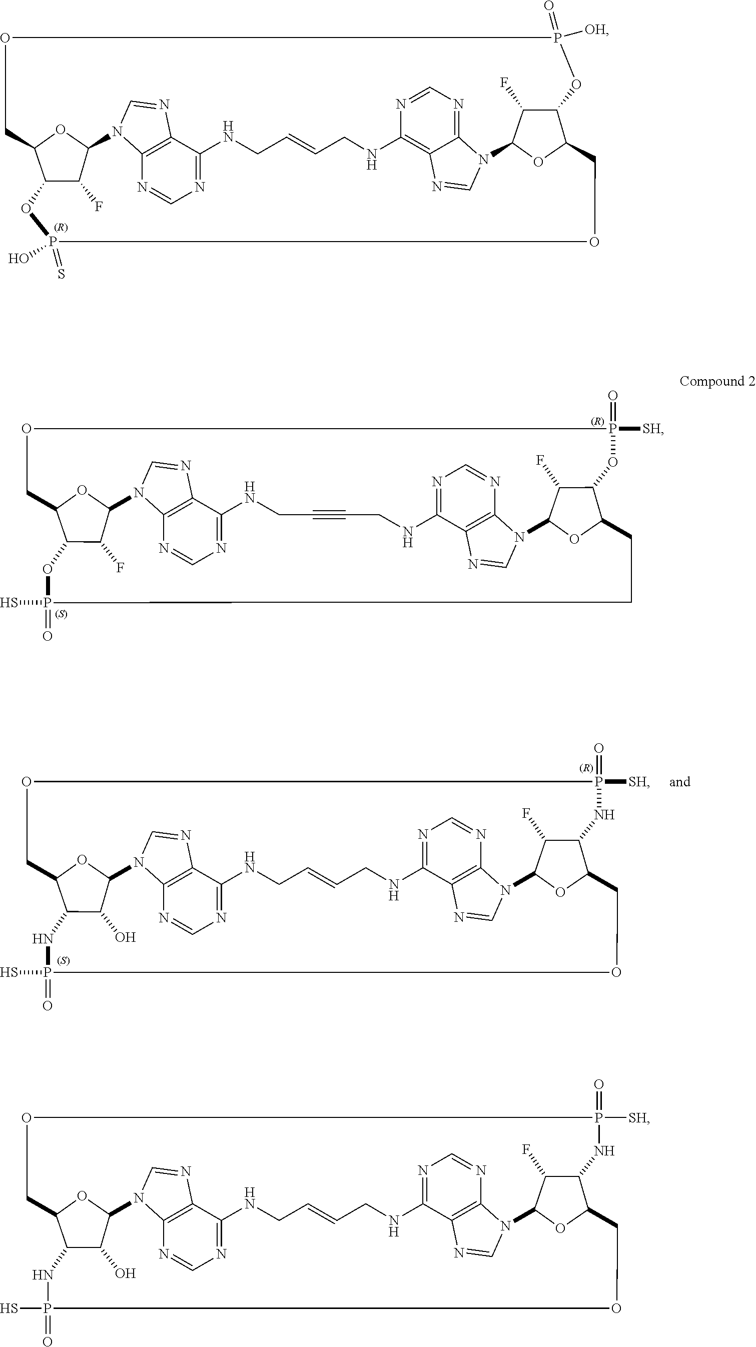
and salts thereof.

28. The linker-payload conjugate of claim 11 or claim 27, wherein D comprises a compound of Formula (III) selected from: 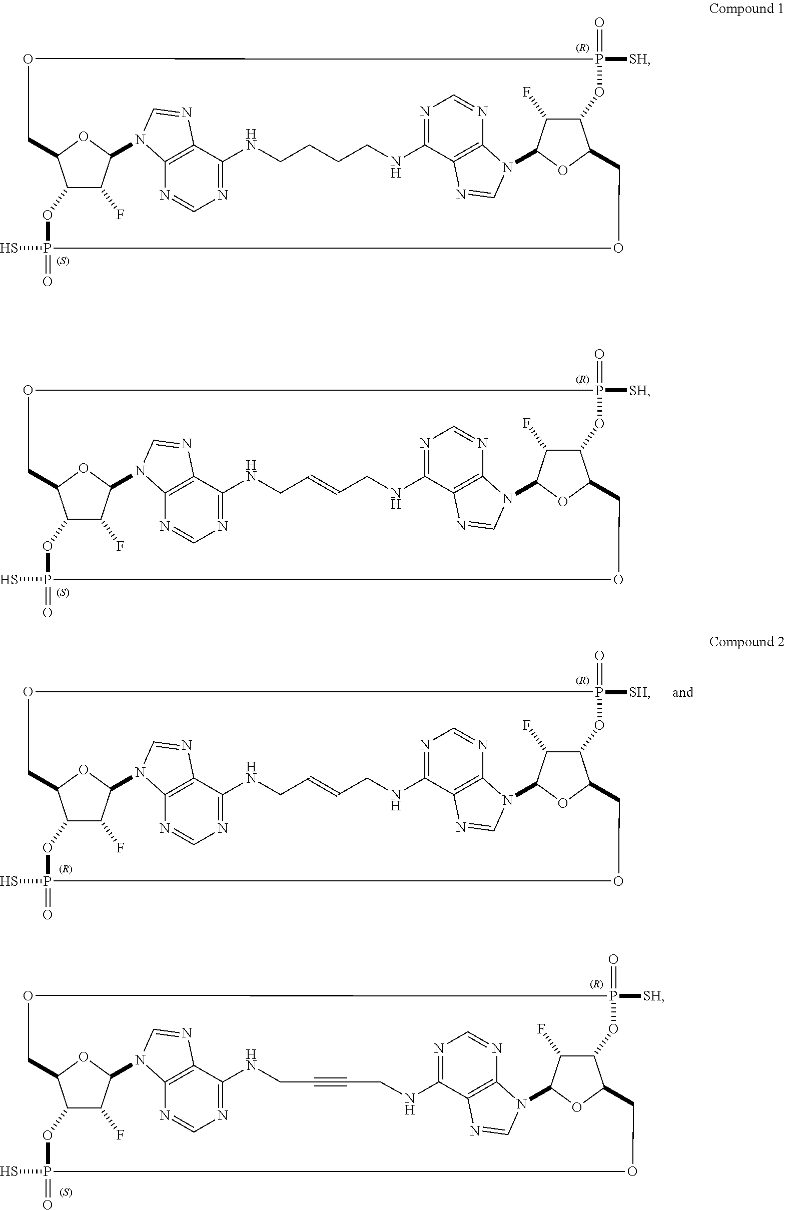
and salts thereof.

29. The linker-payload conjugate of claim 27 or claim 28, wherein D comprises Compound 1.

30. The linker-payload conjugate of claim 27 or claim 28, wherein D comprises Compound 2.

31. The linker-payload conjugate of any one of claims 11 to 30, wherein at least one of Xₐ and X_{b} is SH and L is attached to D via a sulfur atom at the S-2 sulfur or the S-14 sulfur.

32. The linker-payload conjugate of claim 31, wherein X_{b} is SH and L is attached to D at the S-2 sulfur.

33. The linker-payload conjugate of claim 31, wherein X is SH and L is attached to D at the S-14 sulfur.

34. The linker-payload conjugate of any one of claims 11 to 30, wherein at least one of Zₐ and Z_{b} is NH and L is attached to D via a nitrogen atom at the N-34 nitrogen or the N-39 nitrogen.

35. The linker-payload conjugate of claim 34, wherein Z_{b} is NH and L is attached to D at the N-34 nitrogen.

36. The linker-payload conjugate of claim 34, wherein Zₐ is NH and L is attached to D at the N-39 nitrogen.

37. The linker-payload conjugate of any one of claims 11 to 36, wherein L is a cleavable linker.

38. The linker-payload conjugate of claim 37, wherein the cleavable linker comprises a cleavable peptide moiety.

39. The linker-payload conjugate of claim 38, wherein the cleavable peptide moiety is cleavable by a protease, optionally wherein the protease is a cathepsin or a legumain.

40. The linker-payload conjugate of claim 38 or claim 39, wherein the cleavable peptide moiety comprises an amino acid unit.

41. The linker-payload conjugate of claim 40, wherein the amino acid unit comprises Val-Ala, Val-Cit, Val-Lys, Ala-Ala-Asn, Ala-(NMe)Ala-Asn, Asn, Gly-Gly-Phe-Gly, Glu-Val-Ala, or Gly-Val-Ala.

42. The linker-payload conjugate of any one of claims 37 to 41, wherein the cleavable linker comprises Val-Ala.

43. The linker-payload conjugate of any one of claims 11 to 42, wherein the linker comprises a maleimide (Mal) moiety.

44. The linker-payload conjugate of claim 43, wherein the Mal moiety comprises maleimidocaproyl (MC).

45. The linker-payload conjugate of claim 43 or claim 44, wherein the Mal moiety is joined to an antibody or antigen-binding fragment via a cysteine residue on the antibody or antigen-binding fragment.

46. The linker-payload conjugate of any one of claims 11 to 45, wherein the linker further comprises at least one spacer unit.

47. The linker-payload conjugate of claim 46, wherein the at least one spacer unit comprises at least one polyethylene glycol (PEG) moiety.

48. The linker-payload conjugate of claim 47, wherein the at least one PEG moiety comprises -(PEG)ₘ- and m is an integer from 1 to 10.

49. The linker-payload conjugate of claim 48, wherein m is an integer from 2 to 8.

50. The linker-payload conjugate of claim 48 or claim 49, wherein m is an integer from 2 to 5.

51. The linker-payload conjugate of any one of claims 48 to 50, wherein m is 2.

52. The linker-payload conjugate of claim 46 or claim 47, wherein the at least one spacer unit comprises PEG₂-Lys(ε-PEG₈-OMe)-PEG₂.

53. The linker-payload conjugate of claim 46, wherein the at least one spacer unit comprises

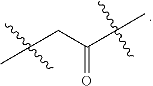 [SMILES: *CC(*)=O]

54. The linker-payload conjugate of claim 46 or claim 53, wherein the at least one spacer unit 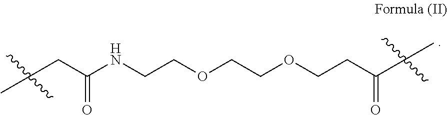 [SMILES: *CC(=O)NCCOCCOCCC(*)=O]
comprises Formula (II).

55. The linker-payload conjugate of any one of claims 11 to 54, wherein the linker further comprises at least one self-immolative unit.

56. The linker-payload conjugate of claim 55, wherein the linker comprises a first self-immolative unit.

57. The linker-payload conjugate of claim 56, wherein the linker is capable of being removed from D after cleavage of the linker by self-immolation of the first self-immolative unit.

58. The linker-payload conjugate of claim 56 or claim 57, wherein the first self-immolative unit comprises a p-aminobenzyl (pAB) optionally substituted with 1-3 substituents chosen from methyl, fluoro, chloro, trifluoromethyl, aryl, and heteroaryl.

59. The linker-payload conjugate of claim 58, wherein the first self-immolative unit comprises a p-aminobenzyl (pAB).

60. The linker-payload conjugate of any one of claims 55 to 59, wherein the linker comprises MC-Val-Ala-pAB.

61. The linker-payload conjugate of any one of claims 56 to 59, wherein the first self-immolative unit comprises a p-aminobenzyloxycarbonyl (pABC).

62. The linker-payload conjugate of any one of claims 56 to 61, wherein the linker further comprises a second self-immolative unit.

63. The linker-payload conjugate of claim 62, wherein the linker is capable of being removed from D after cleavage of the linker by self-immolation of the first self-immolative unit and/or self-immolation of the second self-immolative unit.

64. The linker-payload conjugate of claim 62 or claim 63, wherein the linker is removed from D after cleavage of the linker in a stepwise fashion by self-immolation of the first self-immolative unit and then self-immolation of the second self-immolative unit.

65. The linker-payload conjugate of any one of claims 11 to 41 and 43 to 64, wherein the linker comprises a cleavable linker, a first self-immolative unit, and a second self-immolative unit.

66. The linker-payload conjugate of claim 65, wherein the cleavable linker comprises Val-Ala.

67. The linker-payload conjugate of claim 65, wherein the cleavable linker comprises Val-Cit.

68. The linker-payload conjugate of any one of claims 65 to 67, wherein the cleavable linker comprises Formula (II).

69. The linker-payload conjugate of claim 65, wherein the second self-immolative unit comprises one of the following moieties:

| Second Self- | |
|---|---|
| immolative Unit | Chemical Structure |
| Unit 1 (MEC) | 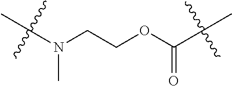 [SMILES: *C(=O)OCCN(*)C] |
| Unit 2 | 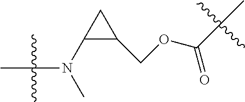 [SMILES: *C(=O)OCC1CC1N(*)C] |
| Unit 3 | 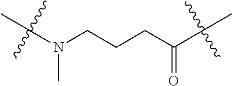 [SMILES: *C(=O)CCCN(*)C] |
| Unit 4 | 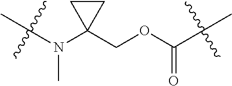 [SMILES: *C(=O)OCC1(N(*)C)CC1] |
| Unit 5 | 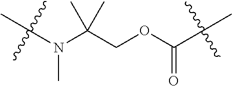 [SMILES: *C(=O)OCC(C)(C)N(*)C] |
| Unit 6 | 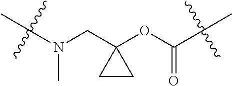 [SMILES: *C(=O)OC1(CN(*)C)CC1] |
| Unit 7 | 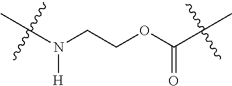 [SMILES: *NCCOC(*)=O] |
| Unit 8 | 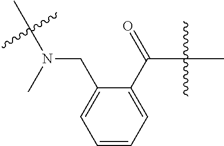 [SMILES: *C(=O)c1ccccc1CN(*)C] |
| Unit 9 | 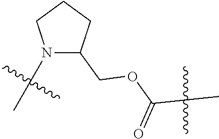 [SMILES: *C(=O)OCC1CCCN1*] |
| Unit 10 | 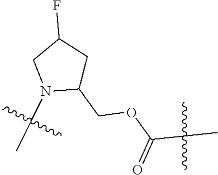 [SMILES: *C(=O)OCC1CC(F)CN1*] |
| Unit 11 | 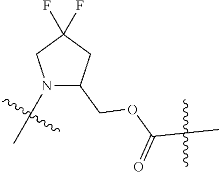 [SMILES: *C(=O)OCC1CC(F)(F)CN1*] |
| Unit 12 | 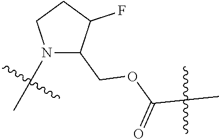 [SMILES: *C(=O)OCC1C(F)CCN1*] |
| Unit 13 | 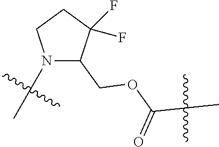 [SMILES: *C(=O)OCC1N(*)CCC1(F)F] |

or an isomer thereof.

70. The linker-payload conjugate of claim 65, wherein the cleavable linker comprises Val-Ala and wherein the second self-immolative unit comprises one of the following moieties:

| Second Self- | |
|---|---|
| immolative Unit | Chemical Structure |
| Unit 1 (MEC) | 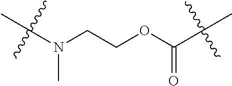 [SMILES: *C(=O)OCCN(*)C] |
| Unit 2 | 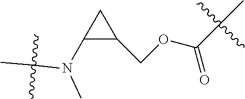 [SMILES: *C(=O)OCC1CC1N(*)C] |
| Unit 3 | 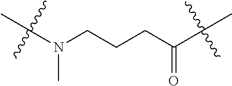 [SMILES: *C(=O)CCCN(*)C] |
| Unit 4 | 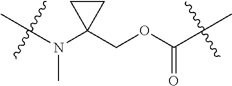 [SMILES: *C(=O)OCC1(N(*)C)CC1] |
| Unit 5 | 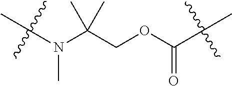 [SMILES: *C(=O)OCC(C)(C)N(*)C] |
| Unit 6 | 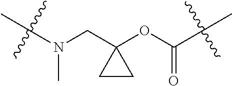 [SMILES: *C(=O)OC1(CN(*)C)CC1] |
| Unit 7 | 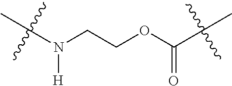 [SMILES: *NCCOC(*)=O] |
| Unit 8 | 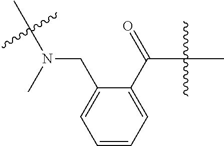 [SMILES: *C(=O)c1ccccc1CN(*)C] |
| Unit 9 | 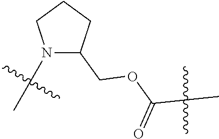 [SMILES: *C(=O)OCC1CCCN1*] |
| Unit 10 | 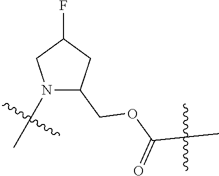 [SMILES: *C(=O)OCC1CC(F)CN1*] |
| Unit 11 | 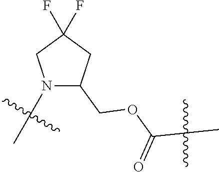 [SMILES: *C(=O)OCC1CC(F)(F)CN1*] |
| Unit 12 | 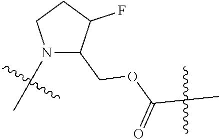 [SMILES: *C(=O)OCC1C(F)CCN1*] |
| Unit 13 | 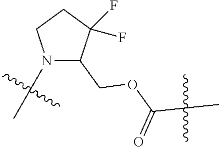 [SMILES: *C(=O)OCC1N(*)CCC1(F)F] |

or an isomer thereof.

71. The linker-payload conjugate of any one of claims 62 to 70, wherein the second self-immolative unit comprises a Unit 1 (MEC) moiety.

72. The linker-payload conjugate of any one of claims 62 to 70, wherein the second self-immolative unit comprises a Unit 8 moiety.

73. The linker-payload conjugate of any one of claims 62 to 70, wherein the second self-immolative unit comprises a Unit 11 moiety.

74. The linker-payload conjugate of any one of claims 62 to 70, wherein the second self-immolative unit comprises a Unit 9 moiety.

75. The linker-payload conjugate of claim 70, wherein the linker comprises Val-Ala-pABC-MEC moiety.

76. The linker-payload conjugate of claim 70, wherein the linker comprises MC-Val-Ala-pABC-MEC moiety.

77. The linker-payload conjugate of claim 11, wherein the L-D comprises LP1:

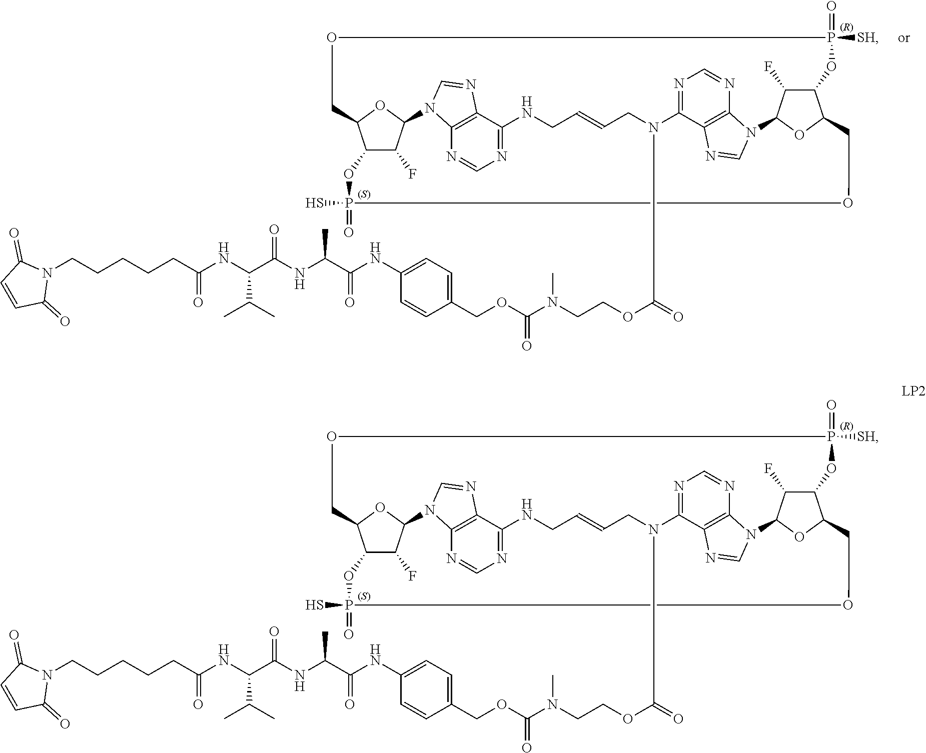

78. The linker-payload conjugate of claim 69, wherein the linker comprises Val-Cit-pABC-MEC moiety.

79. The linker-payload conjugate of claim 69, wherein the linker comprises MC-Val-Cit-pABC-MEC moiety.

80. The linker-payload conjugate of claim 69, wherein the L-D comprises MC-Val-Cit-pABC-MEC-Compound 1.

81. The linker-payload conjugate of claim 70, wherein the linker comprises Val-Ala-pABC-Unit 8 moiety.

82. The linker-payload conjugate of claim 70, wherein the linker comprises MC-Val-Ala-pABC-Unit 8 moiety.

83. The linker-payload conjugate of claim 11, wherein the L-D comprises LP16:

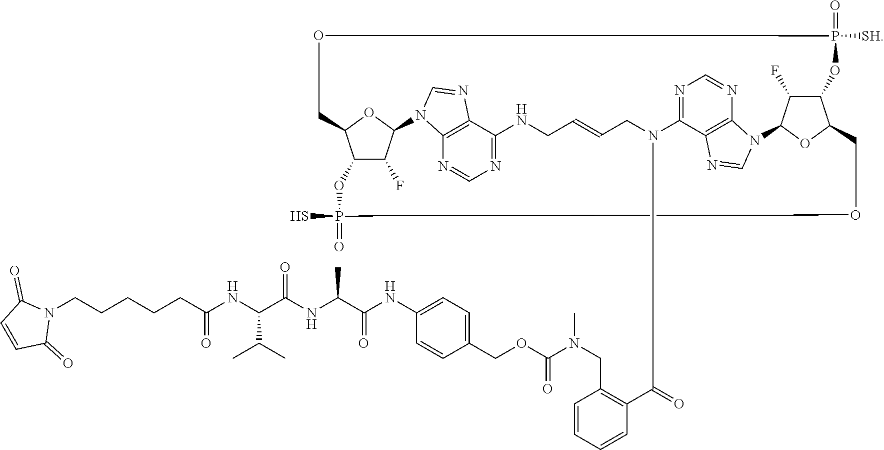 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

84. The linker-payload conjugate of claim 69, wherein the linker comprises Val-Cit-pABC-Unit 8 moiety.

85. The linker-payload conjugate of claim 69, wherein the linker comprises MC-Val-Cit-pABC-Unit 8 moiety.

86. The linker-payload conjugate of claim 69, wherein the L-D comprises MC-Val-Cit-pABC-Unit 8-Compound 1.

87. The linker-payload conjugate of claim 70, wherein the linker comprises Val-Ala-pABC-Unit 11 moiety.

88. The linker-payload conjugate of claim 70, wherein the linker comprises MC-Val-Ala-pABC-Unit 11 moiety.

89. The linker-payload conjugate of claim 11, wherein the L-D comprises LP28:

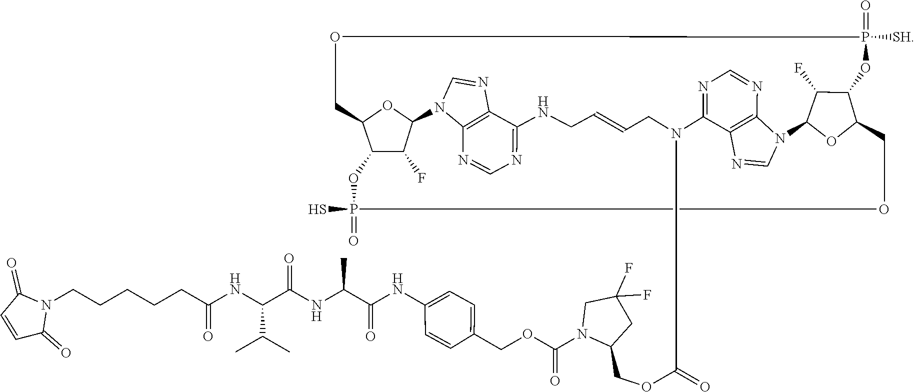 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

90. The linker-payload conjugate of claim 69, wherein the linker comprises Val-Cit-pABC-Unit 11 moiety.

91. The linker-payload conjugate of claim 69, wherein the linker comprises MC-Val-Cit-pABC-Unit 11 moiety.

92. The linker-payload conjugate of claim 69, wherein the L-D comprises MC-Val-Cit-pABC-Unit 11-Compound 1.

93. The linker-payload conjugate of claim 70, wherein the linker comprises Val-Ala-pABC-Unit 9 moiety.

94. The linker-payload conjugate of claim 70, wherein the linker comprises MC-Val-Ala-pABC-Unit 9 moiety.

95. The linker-payload conjugate of claim 11, wherein the L-D comprises LP20:

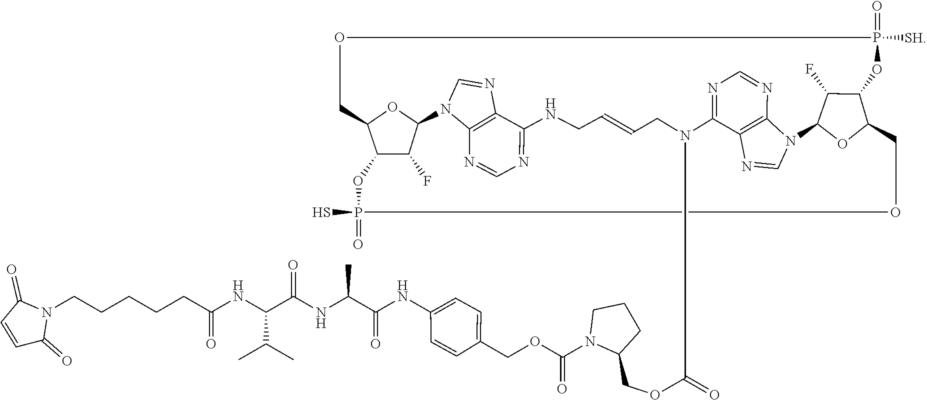 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

96. The linker-payload conjugate of claim 69, wherein the linker comprises Val-Cit-pABC-Unit 9 moiety.

97. The linker-payload conjugate of claim 69, wherein the linker comprises MC-Val-Cit-pABC-Unit 9 moiety.

98. The linker-payload conjugate of claim 69, wherein the L-D comprises MC-Val-Cit-pABC-Unit 9-Compound 1.

99. The linker-payload conjugate of claim 69, wherein the linker comprises Formula (II)-Val-Cit-pABC.

100. The linker-payload conjugate of claim 69, wherein the linker comprises Formula (II)-Val-Cit-pABC-MEC moiety.

101. The linker-payload conjugate of claim 69, wherein the linker comprises Mal-Formula (II)-Val-Cit-pABC-MEC moiety.

102. The linker-payload conjugate of claim 69, wherein the L-D comprises Mal-Formula (II)-Val-Cit-pABC-MEC-Compound 1.

103. The linker-payload conjugate of claim 69, wherein the linker comprises Formula (II)-Val-Cit-pABC-Unit 8 moiety.

104. The linker-payload conjugate of claim 69, wherein the linker comprises Mal-Formula (II)-Val-Cit-pABC-Unit 8 moiety.

105. The linker-payload conjugate of claim 69, wherein the L-D comprises Mal-Formula (II)-Val-Cit-pABC-Unit 8-Compound 1.

106. The linker-payload conjugate of claim 69, wherein the linker comprises Formula (II)-Val-Cit-pABC-Unit 11 moiety.

107. The linker-payload conjugate of claim 69, wherein the linker comprises Mal-Formula (II)-Val-Cit-pABC-Unit 11 moiety.

108. The linker-payload conjugate of claim 69, wherein the L-D comprises Mal-Formula (II)-Val-Cit-pABC-Unit 11-Compound 1.

109. The linker-payload conjugate of claim 69, wherein the linker comprises Formula (II)-Val-Cit-pABC-Unit 9 moiety.

110. The linker-payload conjugate of claim 69, wherein the linker comprises Mal-Formula (II)-Val-Cit-pABC-Unit 9 moiety.

111. The linker-payload conjugate of claim 69, wherein the L-D comprises Mal-Formula (II)-Val-Cit-pABC-Unit 9-Compound 1.

112. The linker-payload conjugate of claim 70, wherein the linker comprises Formula (II)-Val-Ala-pABC.

113. The linker-payload conjugate of claim 70, wherein the linker comprises Formula (II)-Val-Ala-pABC-MEC moiety.

114. The linker-payload conjugate of claim 70, wherein the linker comprises Mal-Formula (II)-Val-Ala-pABC-MEC moiety.

115. The linker-payload conjugate of claim 70, wherein the L-D comprises Mal-Formula (II)-Val-Ala-pABC-MEC-Compound 1.

116. The linker-payload conjugate of claim 70, wherein the linker comprises Formula (II)-Val-Ala-pABC-Unit 8 moiety.

117. The linker-payload conjugate of claim 70, wherein the linker comprises Mal-Formula (II)-Val-Ala-pABC-Unit 8 moiety.

118. The linker-payload conjugate of claim 70, wherein the L-D comprises Mal-Formula (II)-Val-Ala-pABC-Unit 8-Compound 1.

119. The linker-payload conjugate of claim 70, wherein the linker comprises Formula (II)-Val-Ala-pABC-Unit 11 moiety.

120. The linker-payload conjugate of claim 70, wherein the linker comprises Mal-Formula (II)-Val-Ala-pABC-Unit 11 moiety.

121. The linker-payload conjugate of claim 70, wherein the L-D comprises Mal-Formula (II)-Val-Ala-pABC-Unit 11-Compound 1.

122. The linker-payload conjugate of claim 70, wherein the linker comprises Formula (II)-Val-Ala-pABC-Unit 9 moiety.

123. The linker-payload conjugate of claim 70, wherein the linker comprises Mal-Formula (II)-Val-Ala-pABC-Unit 9 moiety.

124. The linker-payload conjugate of claim 70, wherein the L-D comprises Mal-Formula (II)-Val-Ala-pABC-Unit 9-Compound 1.

125. The linker-payload conjugate of claim 69, wherein the linker comprises Formula (II)-Val-Cit-pAB.

126. The linker-payload conjugate of claim 69, wherein the linker comprises Formula (II)-Val-Cit-pAB-Unit 9 moiety.

127. The linker-payload conjugate of claim 69, wherein the linker comprises Mal-Formula (II)-Val-Cit-pAB.

128. The linker-payload conjugate of claim 69, wherein the linker comprises Mal-Formula (II)-Val-Cit-pAB-Unit 9 moiety.

129. The linker-payload conjugate of claim 69, wherein the L-D comprises Mal-Formula (II)-Val-Cit-pAB-Unit 9-Compound 1.

130. The linker-payload conjugate of claim 70, wherein the linker comprises Formula (II)-Val-Ala-pAB.

131. The linker-payload conjugate of claim 70, wherein the linker comprises Formula (II)-Val-Ala-pAB-Unit 9 moiety.

132. The linker-payload conjugate of claim 70, wherein the linker comprises Mal-Formula (II)-Val-Ala-pAB.

133. The linker-payload conjugate of claim 70, wherein the linker comprises Mal-Formula (II)-Val-Ala-pAB-Unit 9 moiety.

134. The linker-payload conjugate of claim 11, wherein the L-D comprises LP25:

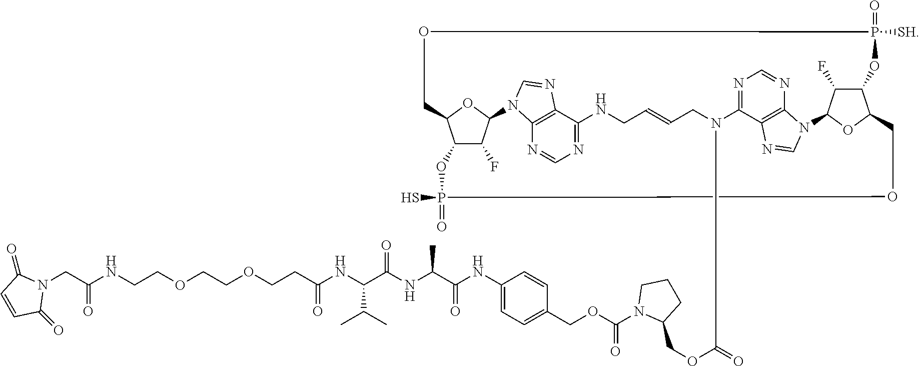 [SMILES: CC(C)[C@H](NC(=O)CCOCCOCCNC(=O)CN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

135. The linker-payload conjugate of claim 69, wherein the linker comprises Formula (II)-Val-Cit-pAB-Unit 11 moiety.

136. The linker-payload conjugate of claim 69, wherein the linker comprises Mal-Formula (II)-Val-Cit-pAB-Unit 11 moiety.

137. The linker-payload conjugate of claim 69, wherein the L-D comprises Mal-Formula (II)-Val-Cit-pAB-Unit 11-Compound 1.

138. The linker-payload conjugate of claim 70, wherein the linker comprises Formula (II)-Val-Ala-pAB-Unit 11 moiety.

139. The linker-payload conjugate of claim 70, wherein the linker comprises Mal-Formula (II)-Val-Ala-pAB-Unit 11 moiety.

140. The linker-payload conjugate of claim 11, wherein the L-D comprises LP26:

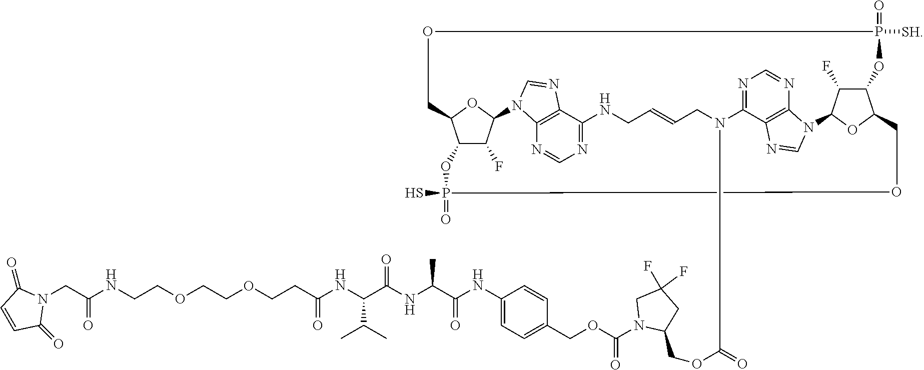 [SMILES: CC(C)[C@H](NC(=O)CCOCCOCCNC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

141. An antibody-drug conjugate of Formula (I): 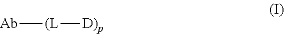
wherein Ab is an anti-PSMA antibody or antigen-binding fragment thereof of any one of claims 1 to 8;
L-D is a linker-payload conjugate of any one of claims 11 to 140; and
p is an integer from 1 to 20.

142. The antibody-drug conjugate of claim 141, wherein p is an integer from 1 to 12, preferably wherein p is an integer from 2 to 8.

143. The antibody-drug conjugate of claim 141 or claim 142, wherein p is an integer from 2 to 4.

144. The antibody-drug conjugate of any one of claims 141 to 143, wherein the linker comprises a cleavable moiety that is positioned such that no part of the linker or the antibody or antigen-binding fragment remains bound to D upon cleavage.

145. The antibody-drug conjugate of any one of claims 141 to 144, wherein the linker-payload conjugate attaches to the antibody or antigen-binding fragment via a Mal moiety, wherein the Mal moiety is joined to the antibody or antigen-binding fragment via a cysteine residue on the antibody or antigen-binding fragment.

146. The antibody-drug conjugate of claim 145, wherein the cysteine residue is on the light chain of the antibody or antigen-binding fragment.

147. The antibody-drug conjugate of claim 145, wherein the cysteine residue is on the heavy chain of the antibody or antigen-binding fragment.

148. The antibody-drug conjugate of any one of claims 141 to 147, wherein the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising amino acid sequences of SEQ ID NO: 32 (LCDR1), SEQ ID NO: 35 (LCDR2), and SEQ ID NO: 37 (LCDR3), as defined by the Kabat numbering system.

149. The antibody-drug conjugate of any one of claims 141 to 147, wherein the antibody or antigen-binding fragment comprises three HCDRs comprising amino acid sequences of SEQ ID NO: 28 (HCDR1), SEQ ID NO: 29 (HCDR2), and SEQ ID NO: 30 (HCDR3); and three LCDRs comprising amino acid sequences of SEQ ID NO: 38 (LCDR1), SEQ ID NO: 39 (LCDR2), and SEQ ID NO: 37 (LCDR3), as defined by the IMGT numbering system.

150. The antibody-drug conjugate of claim 148 or claim 149, wherein the antibody or antigen-binding fragment comprises a heavy chain variable region comprising an amino acid sequence of SEQ ID NO: 14, and a light chain variable region comprising an amino acid sequence of SEQ ID NO: 19.

151. The antibody-drug conjugate of any one of claims 141 to 150, wherein L-D comprises LP16, LP20, LP26, or LP28.

152. The antibody-drug conjugate of any one of claims 141 to 151, wherein the L-D comprises LP16:

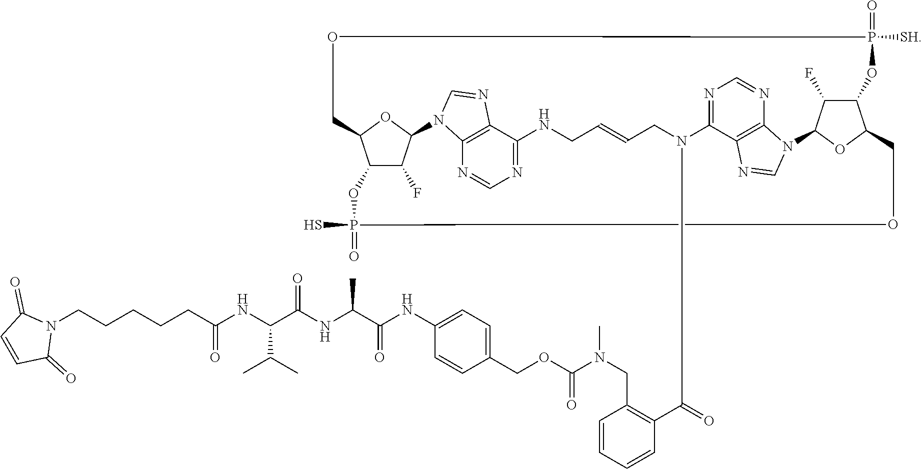 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

153. The antibody-drug conjugate of any one of claims 141 to 151, wherein the L-D comprises LP20:

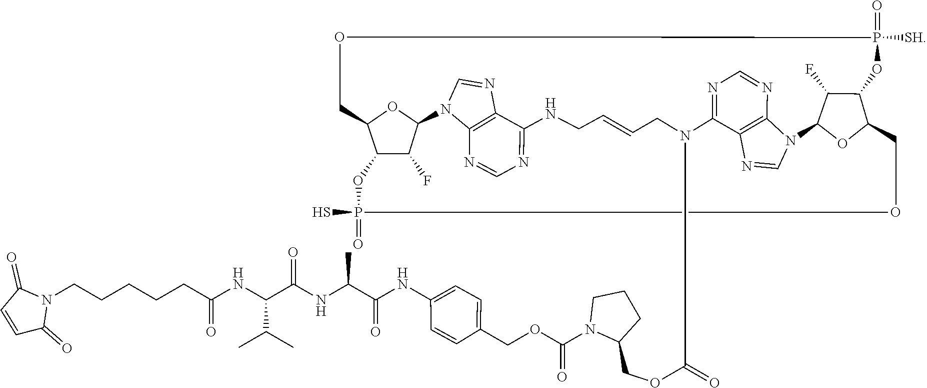 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

154. The antibody-drug conjugate of any one of claims 141 to 151, wherein the L-D comprises LP26:

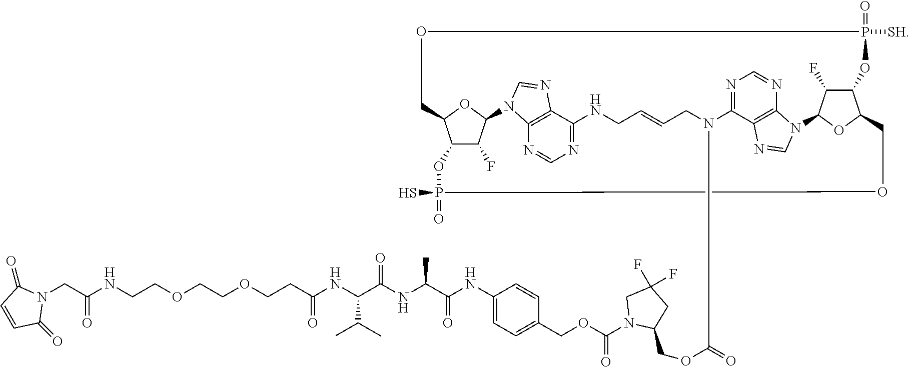

155. The antibody-drug conjugate of any one of claims 141 to 151, wherein the L-D comprises LP28:

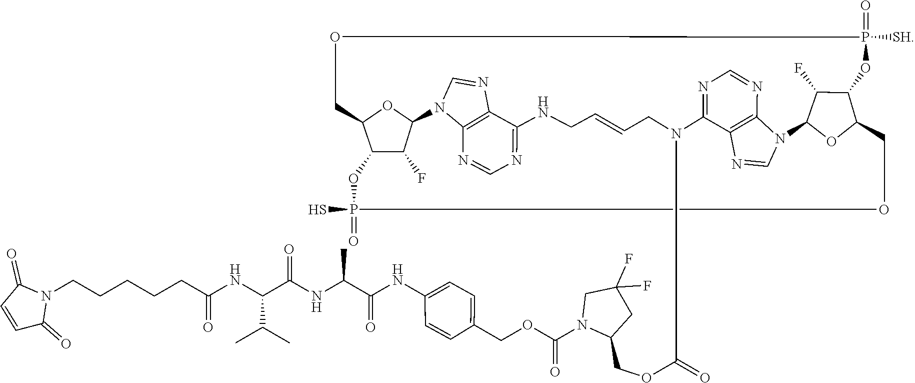 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

156. A pharmaceutical composition comprising the antibody or antigen-binding fragment of any one of claims 1 to 10, the linker-payload conjugate of any one of claims 11-140, or the antibody-drug conjugate of any one of claims 141-155, and a pharmaceutically acceptable carrier.

157. A composition comprising multiple copies of an antibody-drug conjugate of Formula (I): 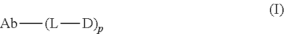
wherein
Ab is an anti-PSMA antibody or antigen-binding fragment thereof of any one of claims 1 to 8;
L-D is a linker-payload conjugate of any one of claims 11 to 140; and
p is the average number of L-D moieties per Ab, wherein the average p of the antibody-drug conjugates in the composition is from about 2 to about 8.

158. The composition of claim 157, wherein
the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP16:

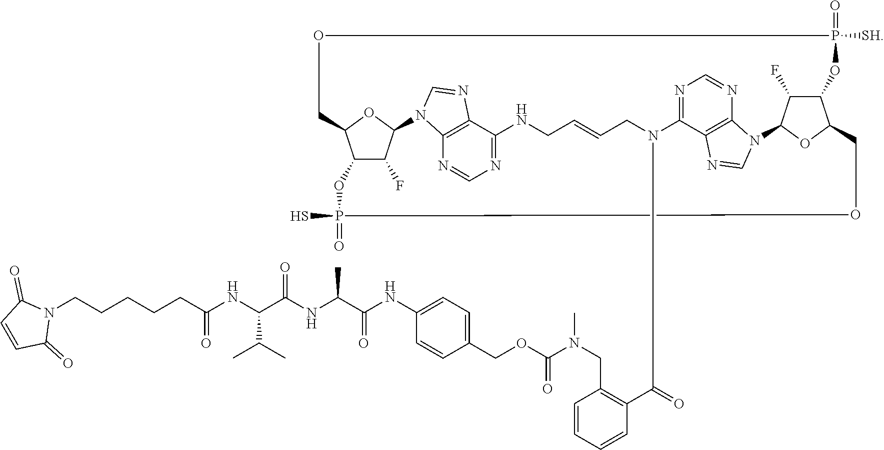 [SMILES: CC(C)[C@H](NC(=O)CCCCCN1C(=O)C=CC1=O)C(=O)N[C@@H](C)C(=O)Nc1ccc(COC(=O)N(C)Cc2ccccc2C(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

159. The composition of claim 157, wherein
the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP20:

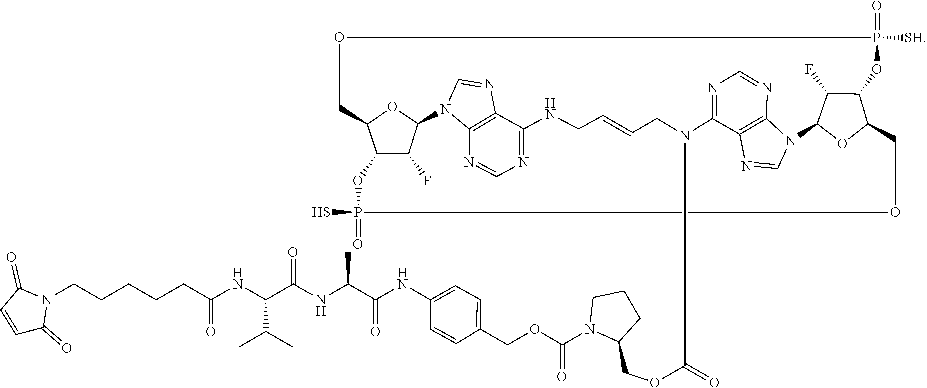 [SMILES: CC(C)[C@H](NC(=O)CCCCCN1C(=O)C=CC1=O)C(=O)N[C@@H](C)C(=O)Nc1ccc(COC(=O)N2CCC[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

160. The composition of claim 157, wherein
the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP26:

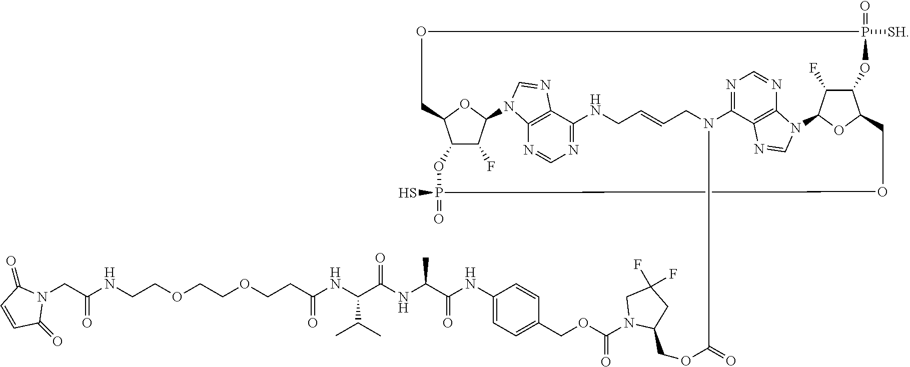

161. The composition of claim 157, wherein
the antibody or antigen-binding fragment comprises three HCDRs comprising amino acid sequences of SEQ ID NO: 21 (HCDR1), SEQ ID NO: 22 (HCDR2), and SEQ ID NO: 27 (HCDR3); and three LCDRs comprising SEQ ID NO: 32 (LCDR1), SEQ ID NO: 35 (LCDR2), and SEQ ID NO: 37 (LCDR3), as defined by the Kabat numbering system; or three HCDRs comprising amino acid sequences of SEQ ID NO: 28 (HCDR1), SEQ ID NO: 29 (HCDR2), and SEQ ID NO: 30 (HCDR3); and three LCDRs comprising SEQ ID NO: 38 (LCDR1), SEQ ID NO: 39 (LCDR2), and SEQ ID NO: 37 (LCDR3), as defined by the IMGT numbering system; and
the L-D comprises LP28:

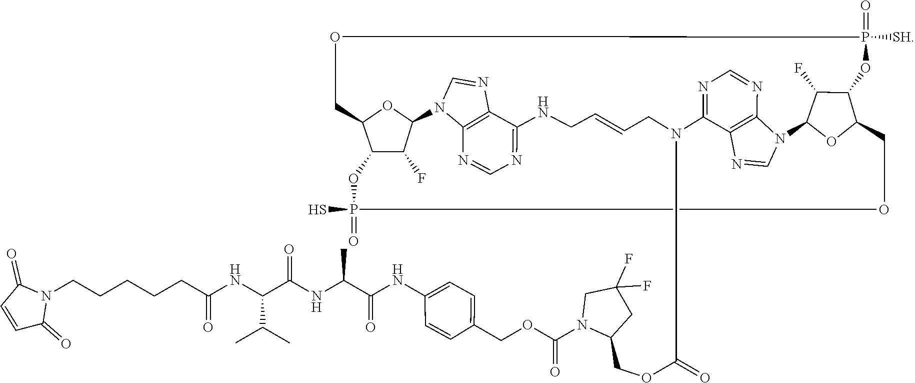 [SMILES: CC(C)[C@H](NC(=O)CCCCCN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1]

162. The composition of any one of claims 157 to 161, wherein the antibody or antigen-binding fragment comprises a heavy chain variable region comprising an amino acid sequence of SEQ ID NO: 14, and a light chain variable region comprising an amino acid sequence of SEQ ID NO: 19.

163. A method of treating a patient having or at risk of having a cancer, comprising administering to the patient a therapeutically effective amount of the antibody or antigen-binding fragment of any one of claims 1 to 10, the linker-payload conjugate of any one of claims 11 to 140, the antibody-drug conjugate of any one of claims 141 to 155, the pharmaceutical composition of claim 156, or the composition of any one of claims 157 to 162.

164. A method of reducing or inhibiting growth of a cancer, comprising administering a therapeutically effective amount of an antibody or antigen-binding fragment of any one of claims 1 to 10, the linker-payload conjugate of any one of claims 11 to 140, the antibody-drug conjugate of any one of claims 141 to 155, the pharmaceutical composition of claim 156, or the composition of any one of claims 157 to 162.

165. Use of an antibody or antigen-binding fragment of any one of claims 1 to 10, the linker-payload conjugate of any one of claims 11 to 140, the antibody-drug conjugate of any one of claims 141 to 155, the pharmaceutical composition of claim 156, or the composition of any one of claims 157 to 162, in the manufacture of a medicament for the treatment of a cancer.

166. Use of an antibody or antigen-binding fragment of any one of claims 1 to 10, the linker-payload conjugate of any one of claims 11 to 140, the antibody-drug conjugate of any one of claims 141 to 155, the pharmaceutical composition of claim 156, or the composition of any one of claims 157 to 162, in the treatment of a cancer.

167. The method of claim 163 or claim 164 or the use of claim 165 or claim 166, wherein the cancer expresses PSMA.

168. The method of claim 163 or claim 164 or the use of claim 165 or claim 166, wherein the cancer is prostate cancer.

169. A method of producing the antibody-drug conjugate of any one of claims 141 to 155 or the composition of any one of claims 157 to 162, comprising reacting an antibody or antigen-binding fragment of any one of claims 1 to 8 with a linker-payload conjugate of any one of claims 11 to 140.

170. An antibody-drug conjugate produced according to the method of claim 169.

171. A method of producing an L-D conjugate (V): 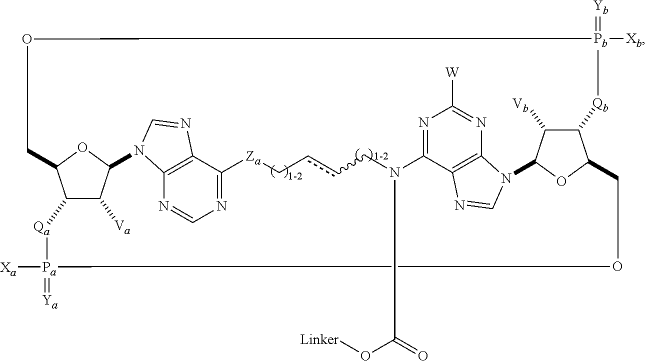
the method comprising reacting a compound of Formula (III) of claim 11: 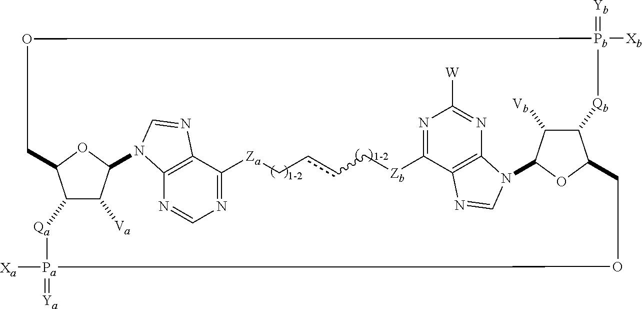
or a salt thereof with an activated linker comprising a suitable linker having the following structure: 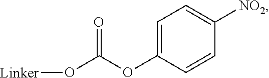
to produce the L-D conjugate (V),
wherein Z_{b} is NH.

172. The method of claim 171, wherein P_{b} has (S)-configuration, and the activated linker reacts with Z_{b} preferentially.

173. A method of producing an L-D conjugate (VI): 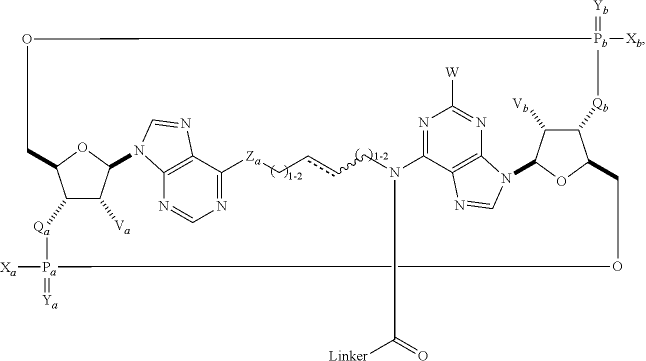
the method comprising reacting a compound of Formula (III) of claim 11: 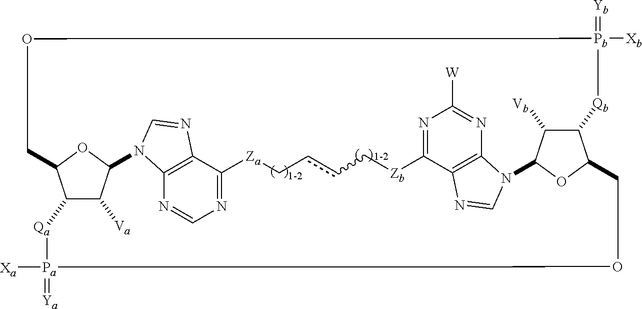
or a salt thereof with an activated linker comprising a suitable linker having the following structure: 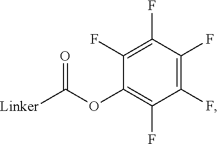
to produce the L-D conjugate (VI),
wherein Z_{b} is NH.

174. The method of claim 173, wherein P_{b} has (S)-configuration, and the activated linker reacts with Z_{b} preferentially.

175. The method of any one of claims 171 to 174, wherein the compound of Formula (III) is Compound 1.

176. A linker-drug conjugate produced by the method of any one of claims 171 to 175.

177. A method of producing an antibody-drug conjugate, wherein the method comprises conjugating the antibody or antigen-binding fragment of any one of claims 1 to 8 with the L-D conjugate of any one of claims 11 to 140 under conditions suitable for attachment.

178. A composition comprising the linker-payload conjugate of any one of claims 11 to 140.

179. A composition comprising a linker-payload conjugate produced according to the method of any one of claims 171 to 175.

180. The linker-payload conjugate of claim 11, wherein the linker-payload conjugate is selected from the following linker-payload conjugates: 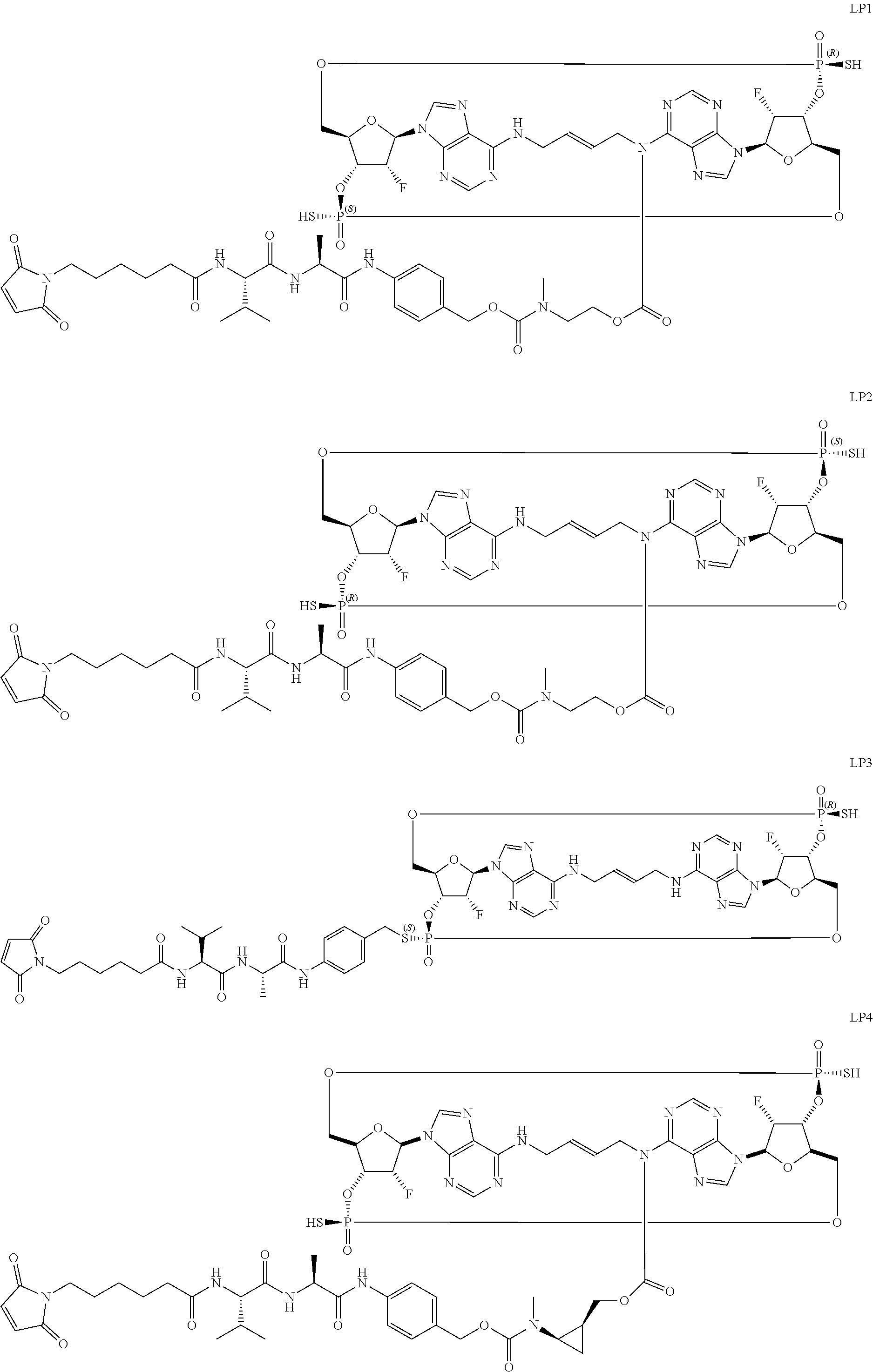 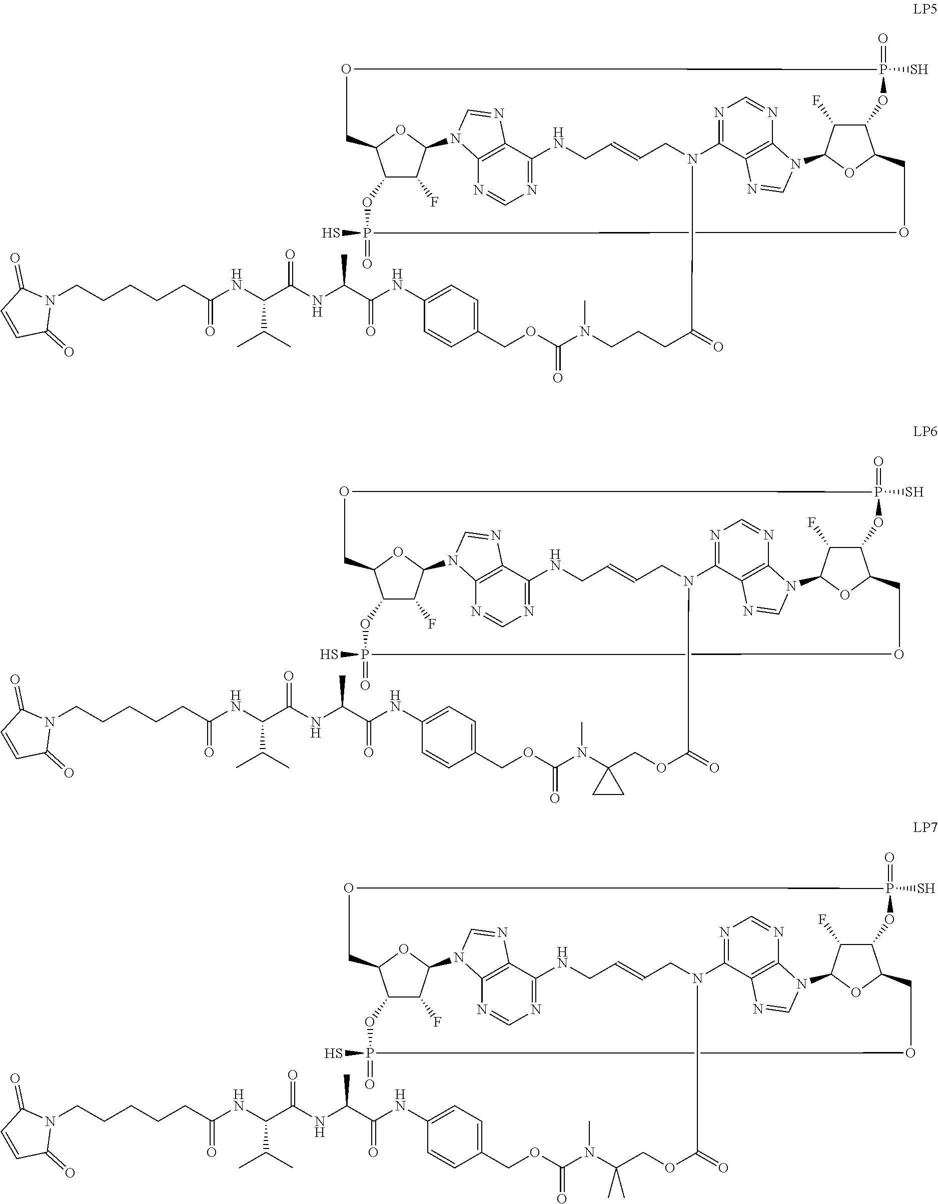 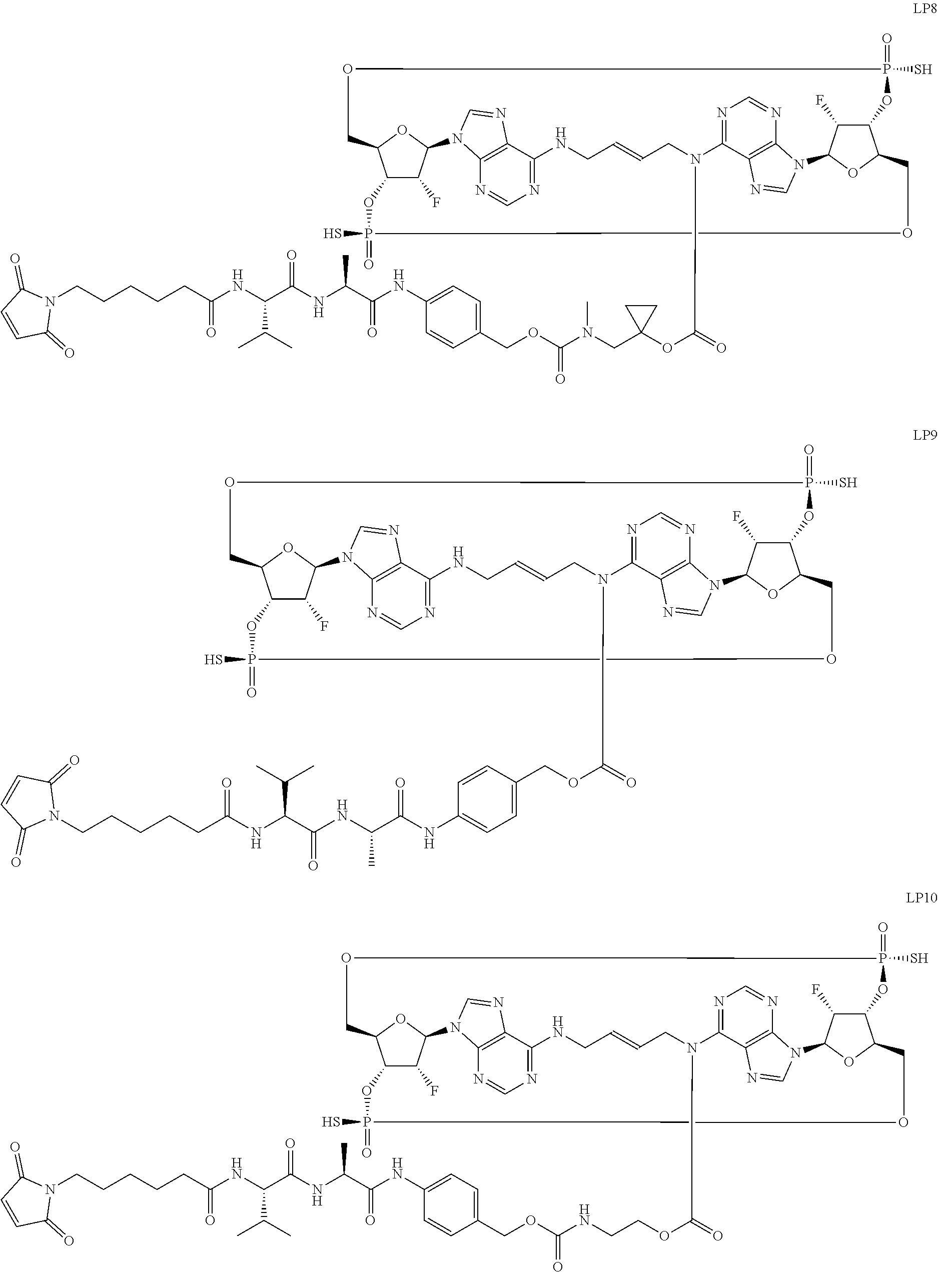 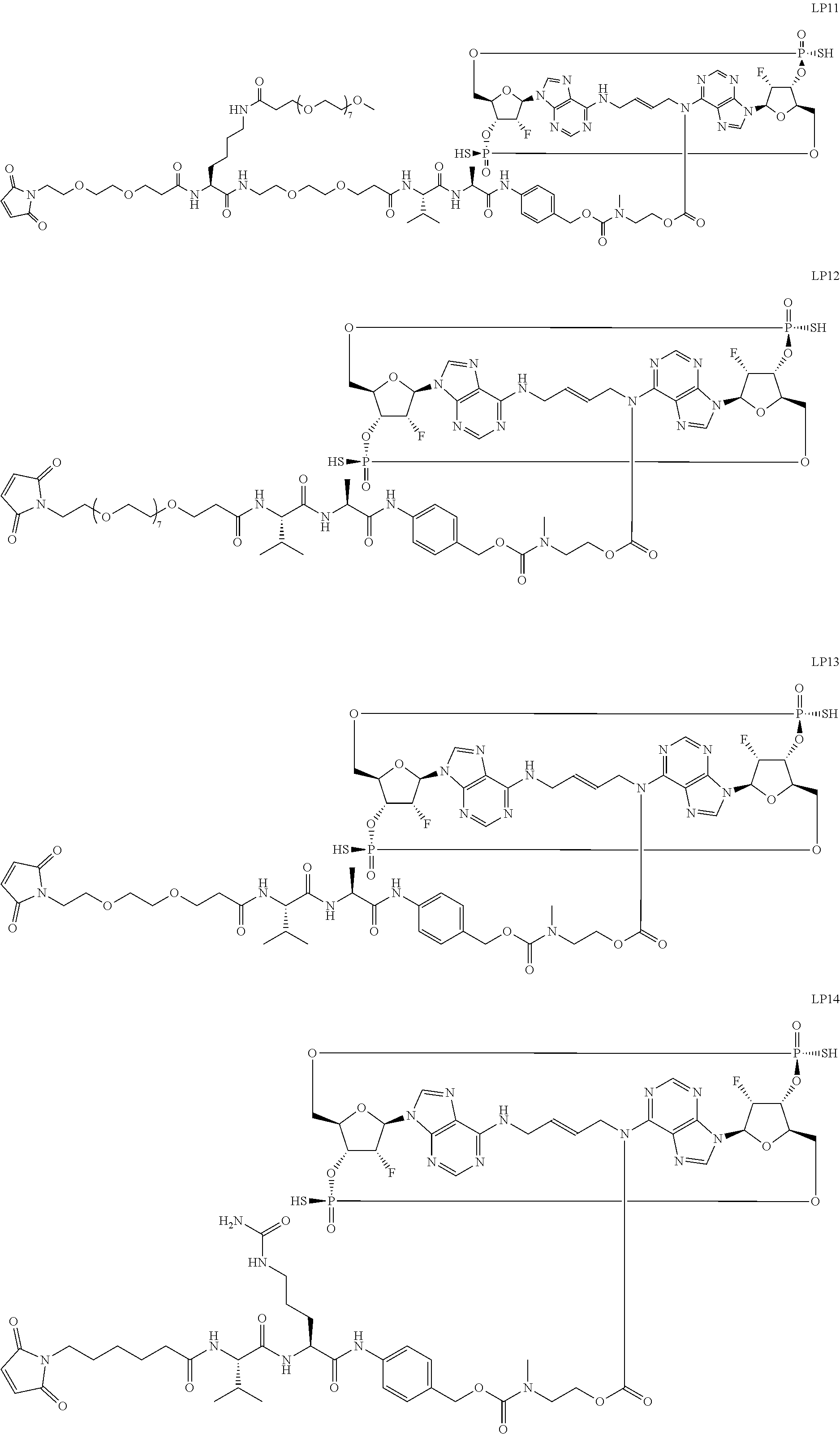 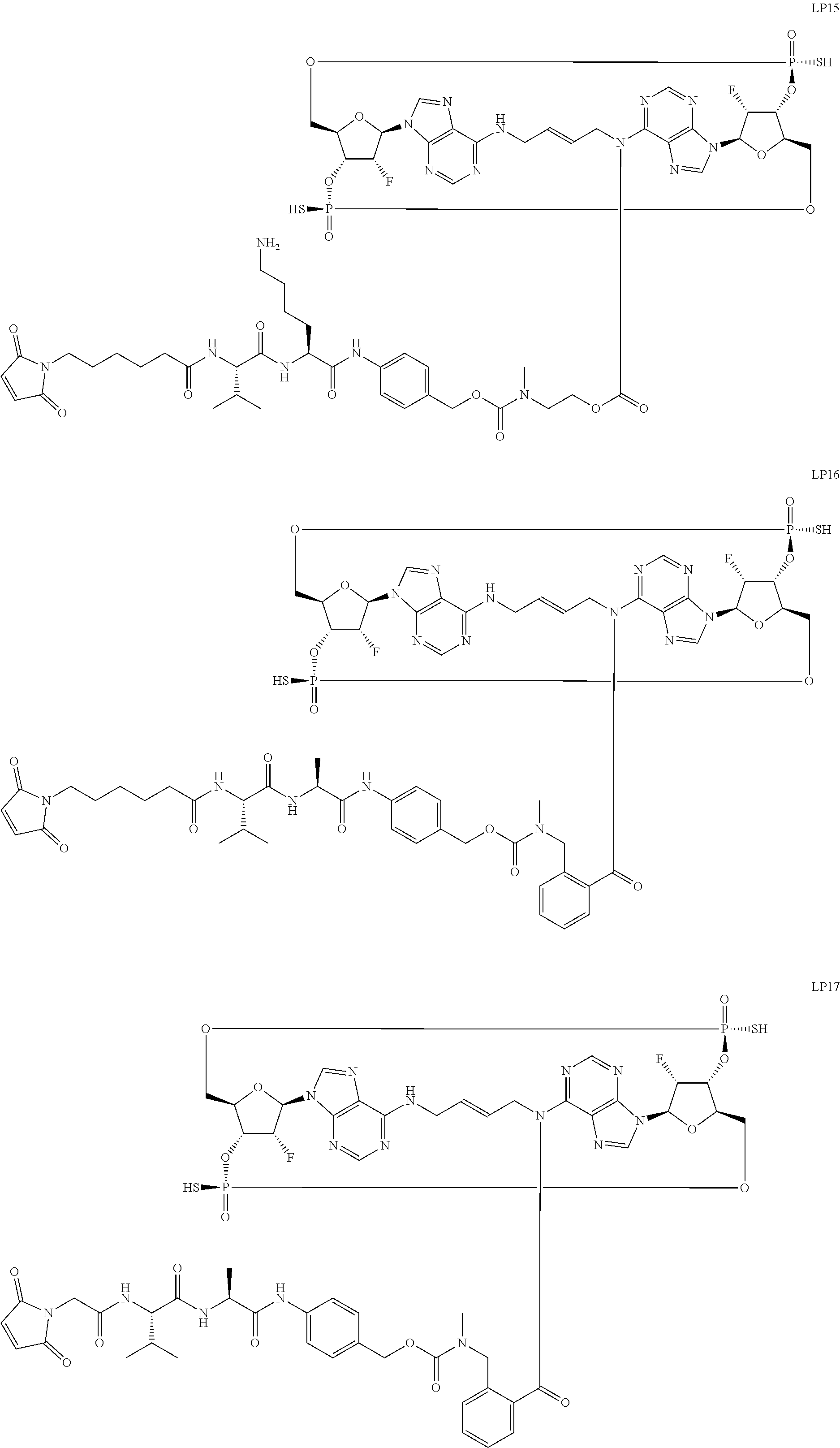 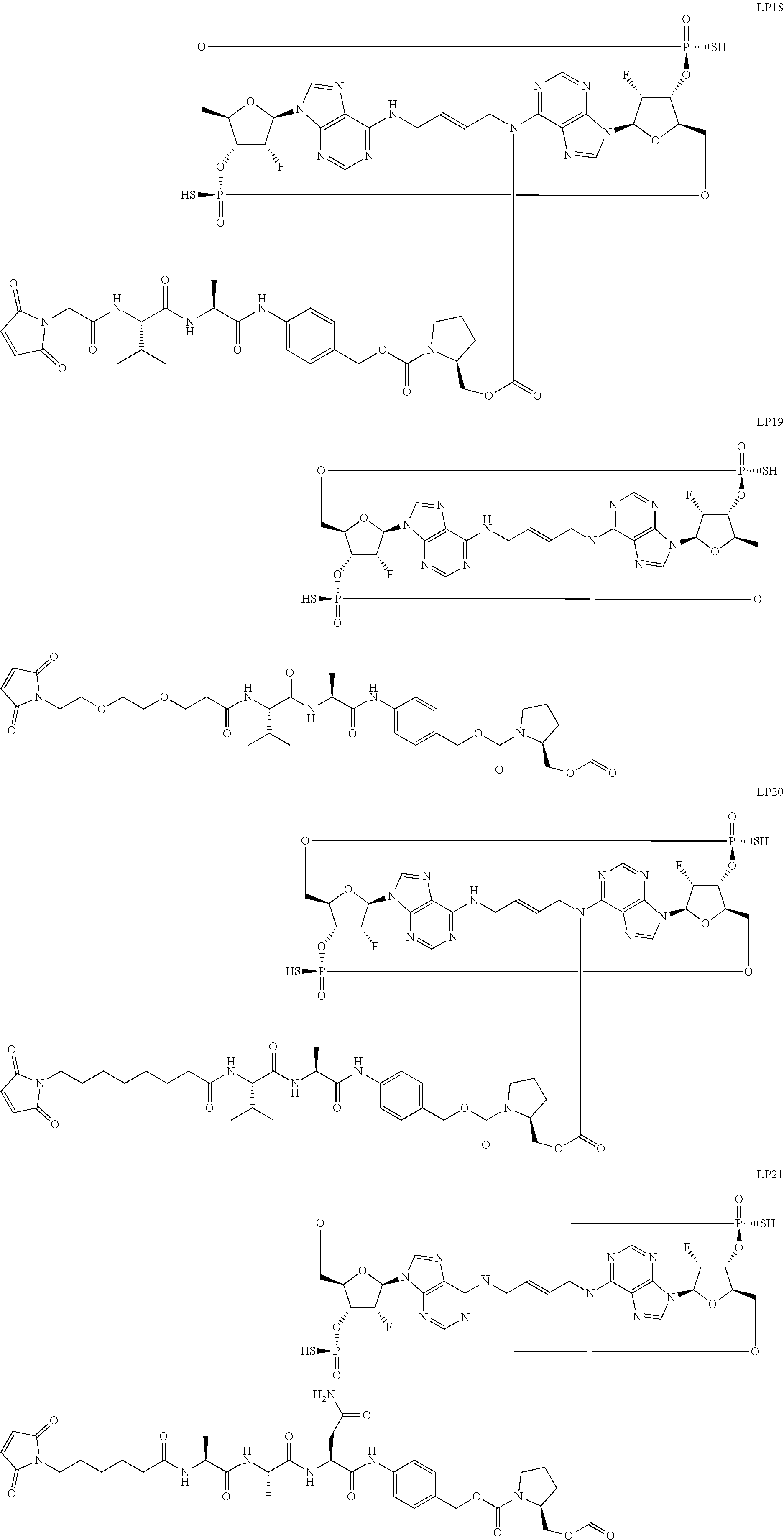 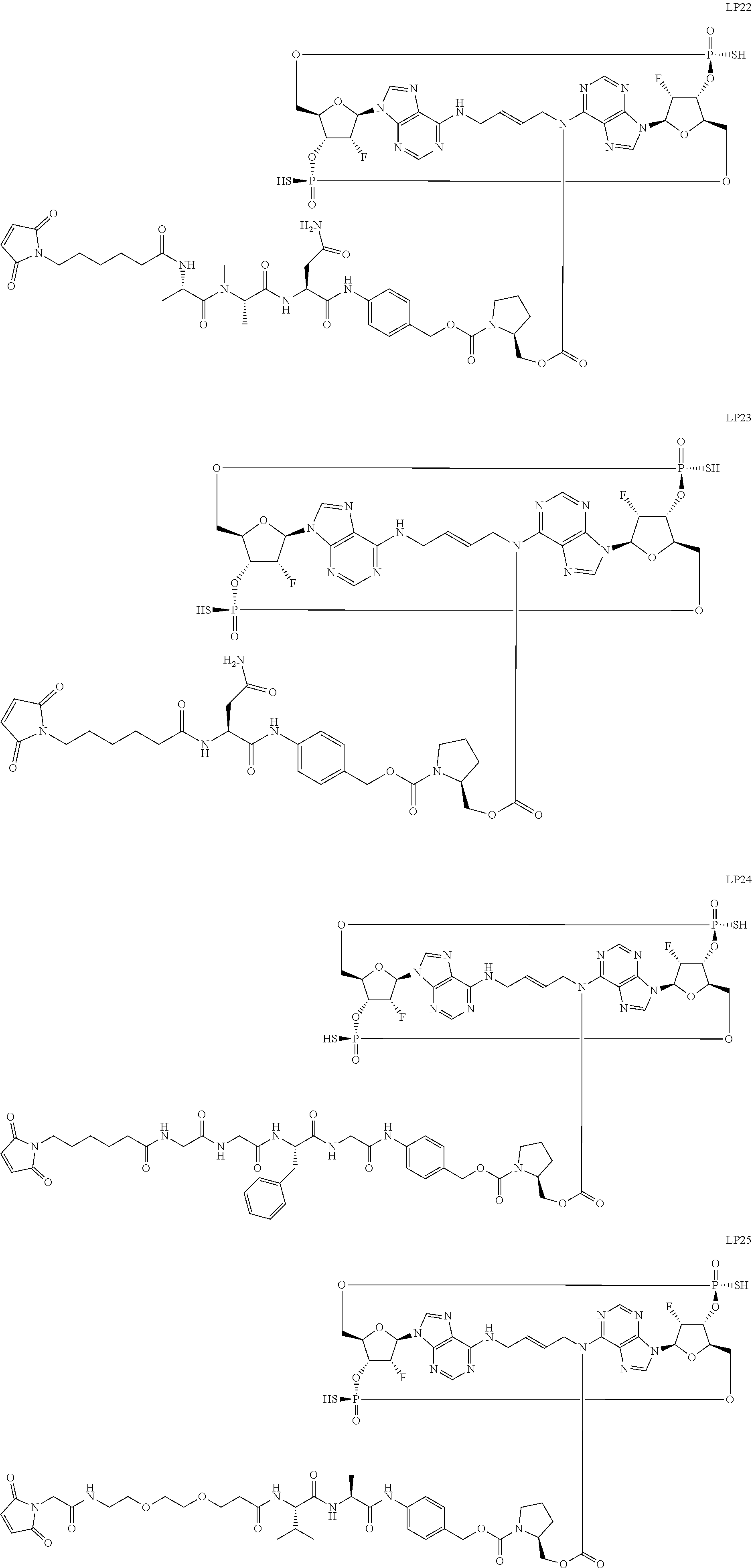 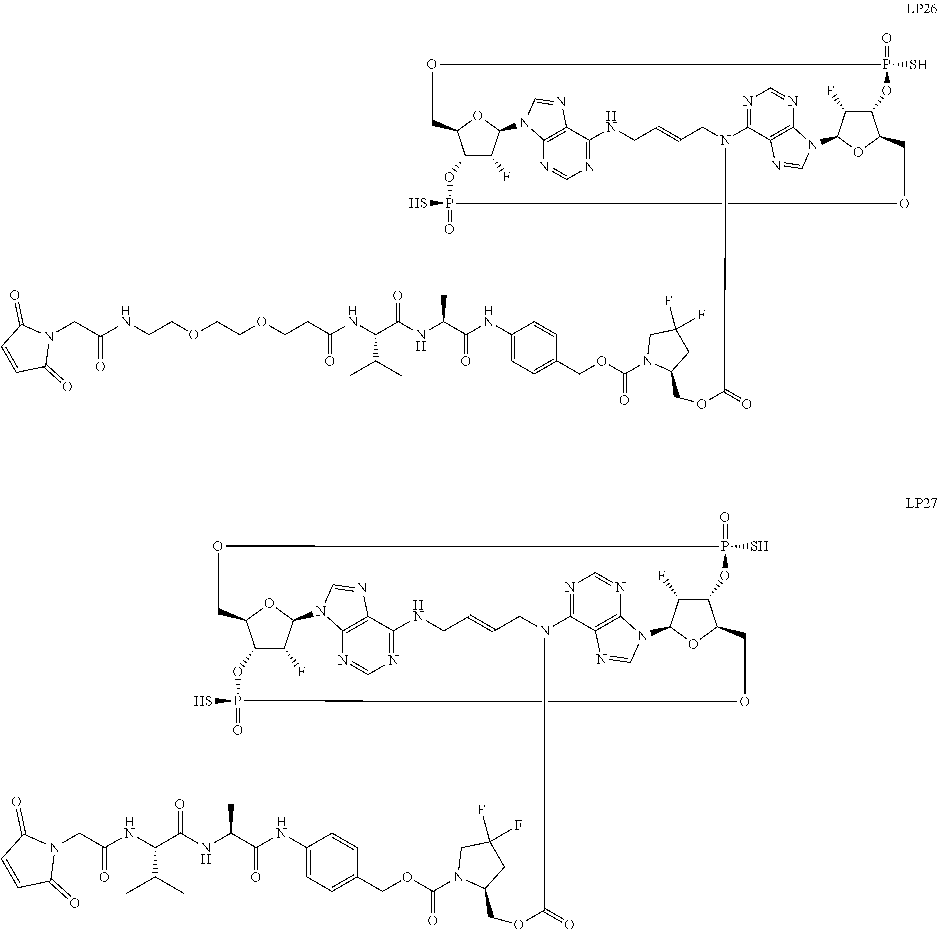 [SMILES: CC(C)[C@H](NC(=O)CCOCCOCCNC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1 ; CC(C)[C@H](NC(=O)CN1C(=O)C=CC1=O)C(=O)N[C@@H](C)C(=O)Nc1ccc(COC(=O)N2CC(F)(F)C[C@H]2COC(=O)N2C/C=C/CNc3ncnc4c3ncn4[C@@H]3O[C@@H]4CO[P@](=O)(S)O[C@H]5[C@@H](F)[C@@H](O[C@@H]5CO[P@@](=O)(S)O[C@H]4[C@H]3F)n3cnc4c2ncnc43)cc1] 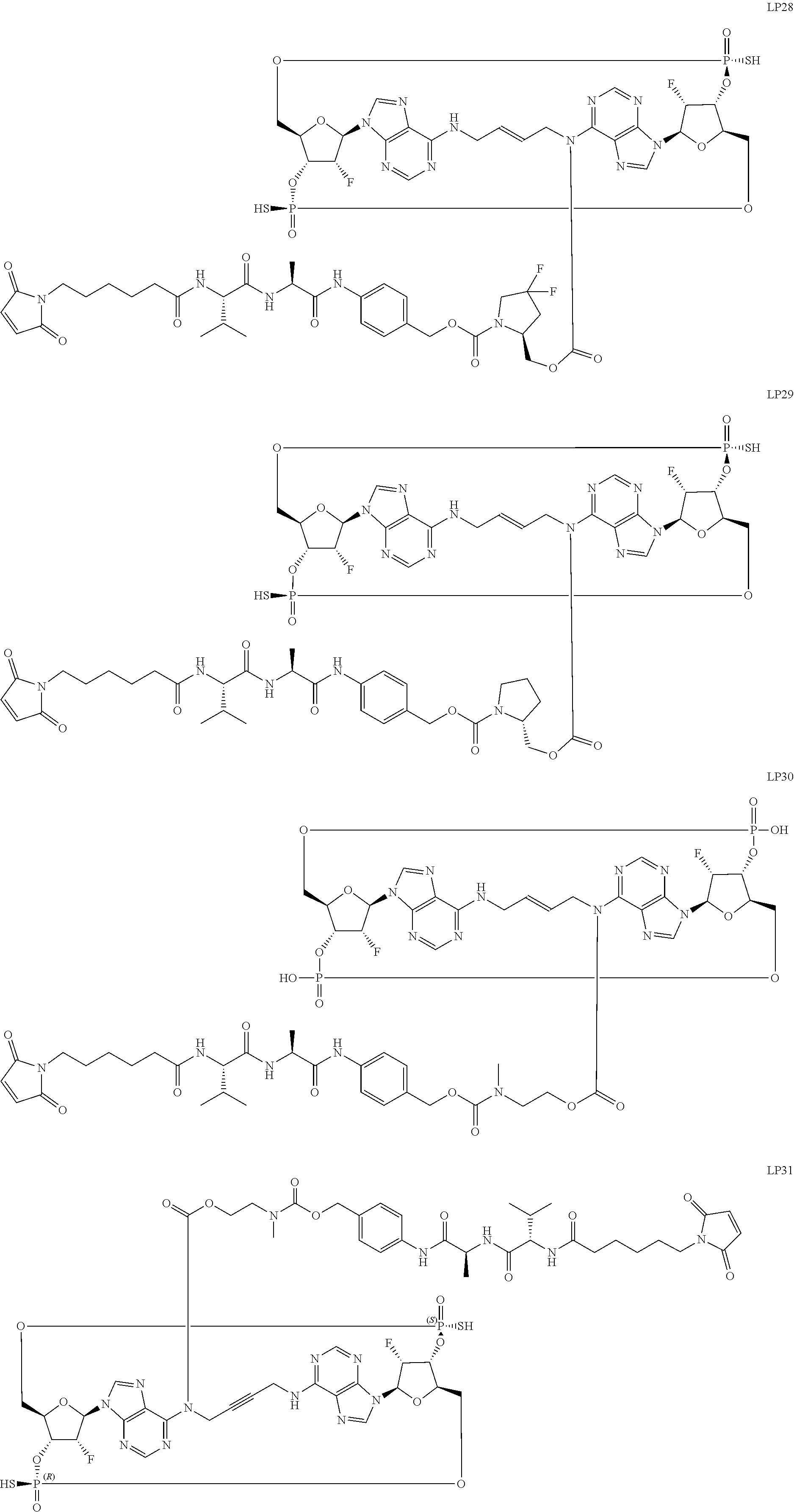 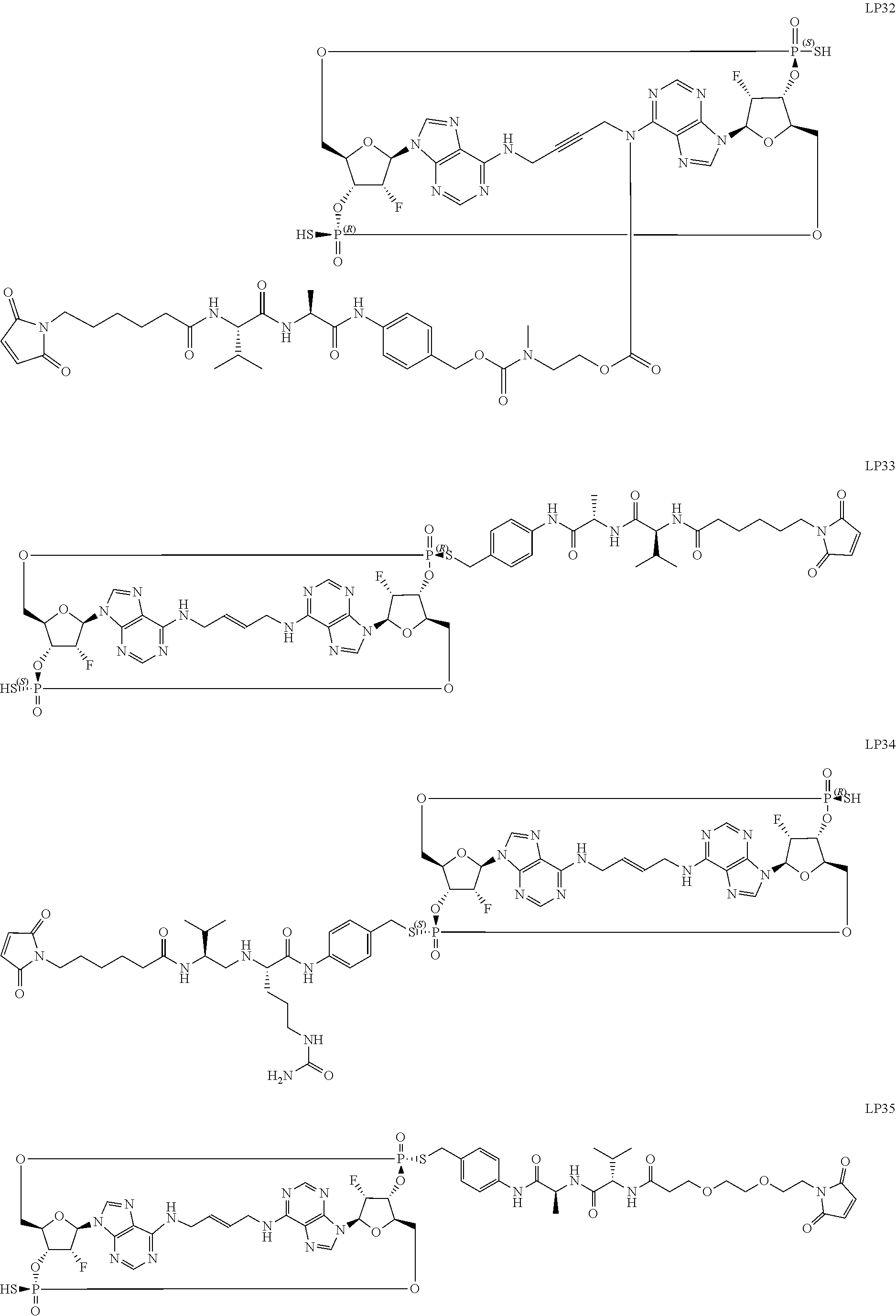 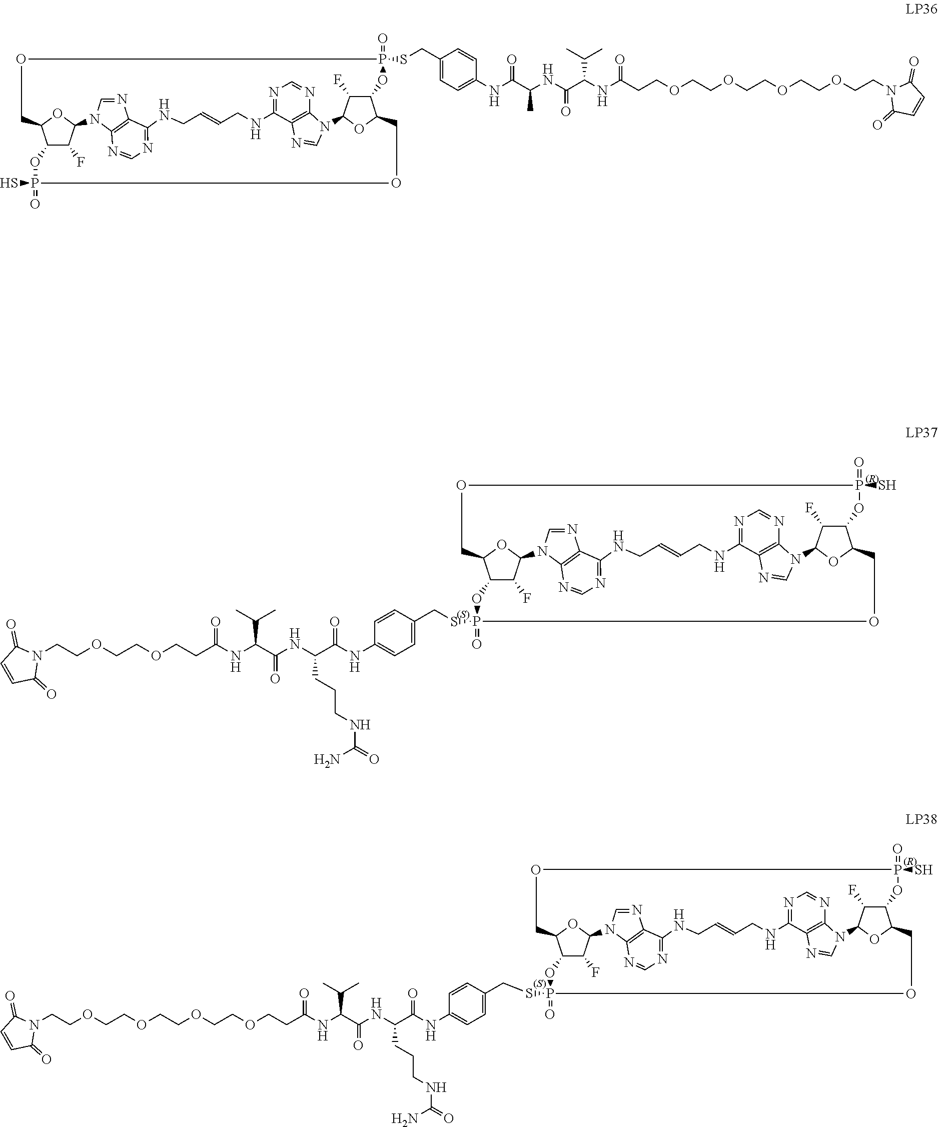 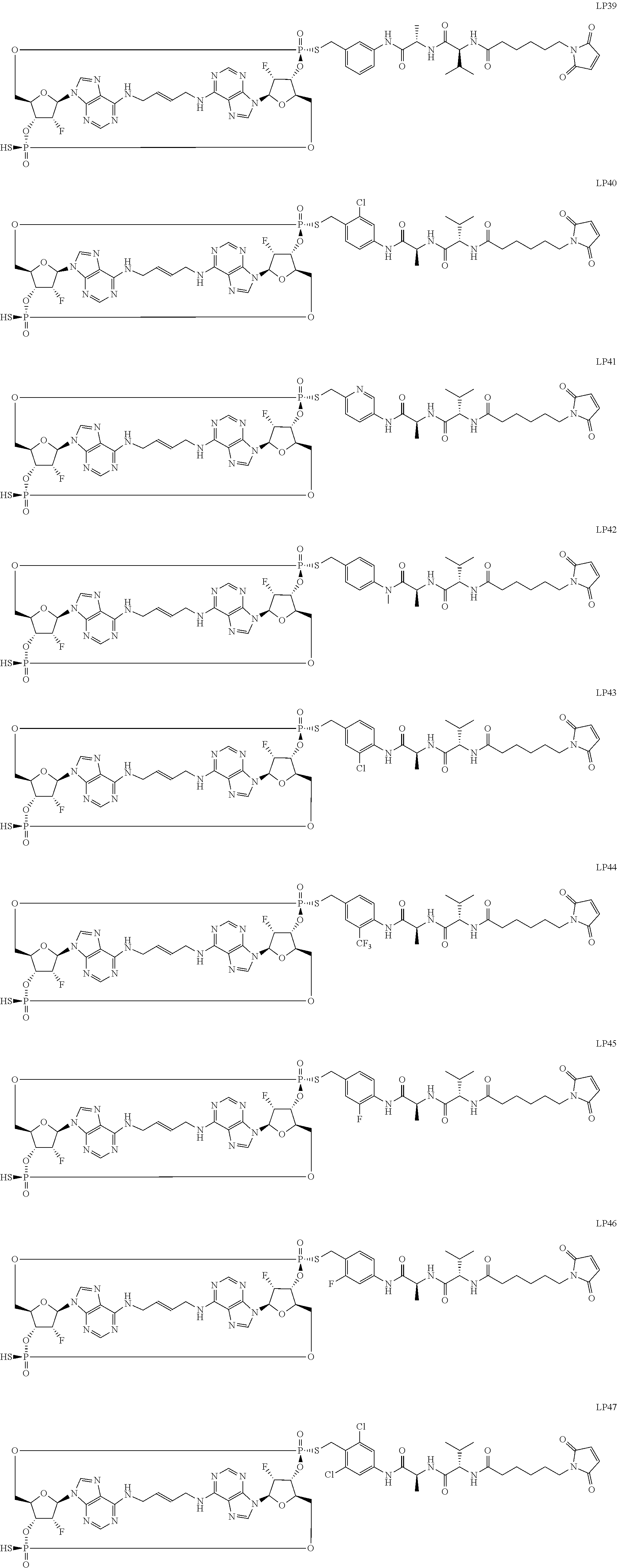 [chemical structure]
